(19) United States
(12) Patent Application Publication
(10) Pub. No.: US 2026/0274856 A1
(43) Pub. Date: Sep. 17, 2026

(54) COVALENT CEREBLON LIGANDS

(71) Applicant: C4 THERAPEUTICS, INC., Watertown, MA (US)

(72) Inventors: Kimberly Muno Hilby, Watertown, MA (US); Xinpeng Cheng, Watertown, MA (US); Jenifer N. Winters, Watertown, MA (US)

(73) Assignee: C4 THERAPEUTICS, INC., Watertown, MA (US)

(21) Appl. No.: 19/664,180

(22) Filed: Apr. 30, 2026

Related U.S. Application Data
(63) Continuation of application No. PCT/US2024/054005, filed on Oct. 31, 2024.
(60) Provisional application No. 63/546,697, filed on Oct. 31, 2023.

(51) Int. Cl. C07D 491/107; A61K 31/4709; A61K 31/517; A61K 47/55; C07D 401/14

(52) U.S. Cl. CPC C07D 491/107; A61K 31/4709; A61K 31/517; A61K 47/55; C07D 401/14

(57) ABSTRACT

This invention provides Degrons and Degraders that covalently bind to cereblon which is a component of the E3 ubiquitin ligase. The Degrons provided herein can be used to modulate the activity of cereblon either alone or as covalently linked to a Tail. In addition, the Degron can be linked to a Targeting Ligand which binds to a Target Protein to provide a Degrader.

### CROSS REFERENCE TO RELATED APPLICATIONS

This application is a continuation of International Patent Application No. PCT/US2024/054005, filed Oct. 31, 2024, which claims the benefit of U.S. provisional application 63/546,697, filed Oct. 31, 2023. The entirety of each of these applications is hereby incorporated by reference for all purposes.

### FIELD OF THE INVENTION

This invention provides Degrons and Degraders that covalently bind to cereblon which is a component of the E3 ubiquitin ligase. The Degrons provided herein can be used to modulate the activity of cereblon either alone or as covalently linked to a Tail. In addition, the Degron can be linked to a Targeting Ligand which binds to a Target Protein to provide a Degrader.

### BACKGROUND OF THE INVENTION

Protein degradation is a highly regulated and essential process that maintains cellular homeostasis. The selective identification and removal of damaged, misfolded, or excess proteins is achieved via the ubiquitin-proteasome pathway (“UPP”). The UPP is central to the regulation of almost all cellular processes, including antigen processing, apoptosis, cell cycling, DNA transcription and repair, differentiation and development, immune response and inflammation. Attachment of multiple ubiquitin molecules by an E3 ubiquitin ligase enzyme to a terminal lysine residue of a protein to be degraded marks the protein for proteasomal processing, where the protein is digested into small peptides and eventually into its constituent amino acids, which subsequently are recycled as building blocks for new proteins. Defective proteasomal degradation has been linked to a variety of clinical disorders including cancer, abnormal cellular proliferation, and cardiovascular diseases, among others.

Cereblon is the enzyme receptor component of the multi-protein complex cullin 4-RING E3 ligase. In successive reactions, a polyubiquitin chain is created on a protein to be degraded by the cereblon-containing E3 ligase. The polyubiquitin tail serves as a recognition marker for the proteasome, which degrades the tagged protein into its amino acid components for recycling.

Research has advanced to try to highjack this complex UPP process by designing molecules that bring together the two necessary parts for ubiquitinating the targeted protein—the E3 ligase enzyme and the target protein to be ubiquitinated. This “heterobifunctional” compound (also called a “Degrader” or a “BiDAC”) is a three-component compound typically consists of 1) a non-covalent binding ligand to a protein to be degraded (“target protein ligand”), 2) a linker, and 3) a non-covalent binding ligand to an E3 ligase enzyme, such as cereblon, that ubiquitinates the protein (“cereblon binder”). One of the properties of non-covalently bound heterobifunctional protein degraders is that after the protein “cargo” has been delivered to the ligase protein complex for ubiquitination, the ubiquitinated protein then goes to the proteasome for degradation and the heterobifunctional compound is returned to repeat the function with another protein, thus acting as a recyclable drug. Therefore, the fact that the heterobifunctional protein degrader is non-covalently bound to both the cereblon of the E3 ligase and the protein to be degraded has been considered an advantage, because one molecule can perform repeatedly, which multiplies its therapeutic effect and mimics a catalyst.

A large number of patents and patent applications have been filed describing heterobifunctional compounds for targeted ubiquitination and degradation of disease-mediating proteins, including but not limited to those filed by C4 Therapeutics, Inc, Arvinas, Inc., Dana-Farber Cancer Institute, Kymera Therapeutics, Foghorn Therapeutics, Novartis, Nurix Therapeutics, Cullgen, Inc. and others.

While progress has been made in the area of the degradation of disease-modulating proteins, more therapeutic agents are needed. Therefore, it is an object of the present invention to provide new compounds for the degradation of disease-modulating proteins.

### SUMMARY OF THE INVENTION

A cereblon binding compound (Degron; a degradation inducing moiety) that covalently binds to cereblon through a sulfonyl fluoride or a sulfonimidoyl fluoride is provided. This compound can be used to treat a disorder mediated by cereblon or mediated by a protein which is degraded by cereblon when the Degron described herein binds to cereblon. In another embodiment, a Degron described herein can be used as an intermediate to synthesize a heterobifunctional compound for targeted protein degradation (a “Degrader” or “BiDAC”). In certain aspects the Degron includes a linking moiety (a Tail) which can react with an appropriately prepared Targeting Ligand or Targeting Ligand precursor to form a Degrader. Degraders are also provided which include a Degron described herein which can be directly attached to a Targeting Ligand or attached to the Targeting Ligand with a Linker.

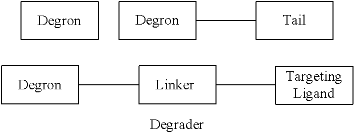

A Degron compound can be a “molecular glue” that can bind to the cereblon E3 ligase thereby creating a new surface on the E3 ligase, resulting in an enhancement of interaction and binding with a targeted protein. As a result of this interaction, the targeted protein may be ubiquitinated by the cereblon E3 ligase and degraded by the proteasome. In some embodiments, the cereblon binding affinity of the Degron enables degradation of the protein associated with a disease, such as, but not limited to, cancer.

For example, a compound of Formula I is a Degron and can thus be used as a therapeutically active compound that changes the surface of cereblon, an intermediate to make a Degrader, or as part of a heterobifunctional compound to degrade a target protein (a Degrader).

The present invention provides a compound of Formula:

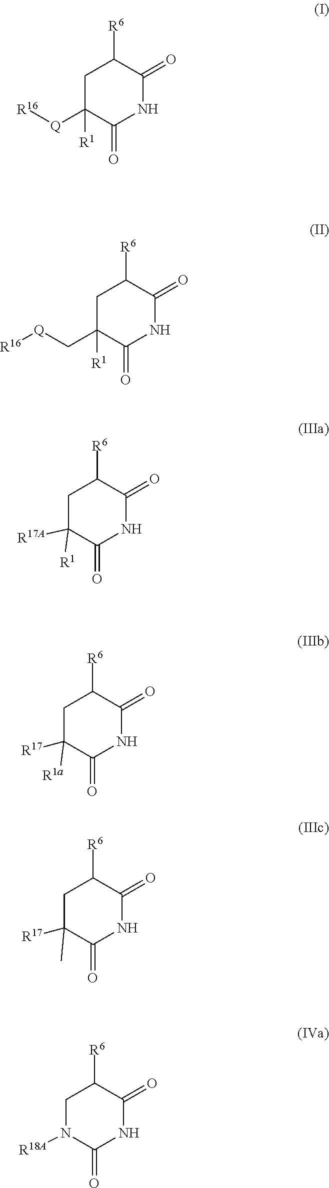

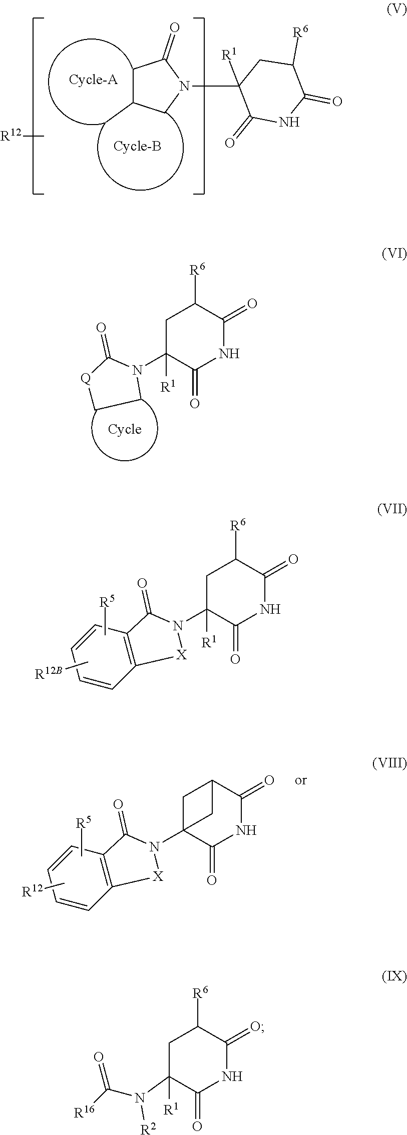

or a pharmaceutically acceptable salt thereof,
wherein:
   Q is CH₂, NR², O, or S;
   in certain embodiments Q is NH, NCH₃, O, or S;
   X is CH₂ or C(O);
   R¹ and R⁶ are independently selected from hydrogen, alkyl, alkenyl, alkynyl, and halogen;
   or R¹ and R⁶ are combined to form a one or two carbon bridge to form a fused cycle, for example

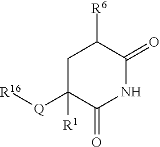

when R¹ and R⁶ are combined to form a one carbon bridge is

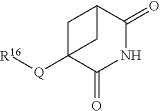

and a two carbon bridge is

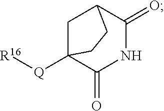

in certain embodiments R¹ and R⁶ are hydrogen;
R^{1a} is independently selected from hydrogen, C₃-C₈ alkyl, alkenyl, alkynyl, and halogen;
or R^{1a} and R⁶ are combined to form a one or two carbon bridge to form a fused cycle;
each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
in certain embodiments each R² is hydrogen or CH₃;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a};
in certain embodiments each R⁵ is selected from CH₃, F, OMe, and NMe₂;
R^{5c} is independently selected from alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a};
R¹⁶ is selected from:

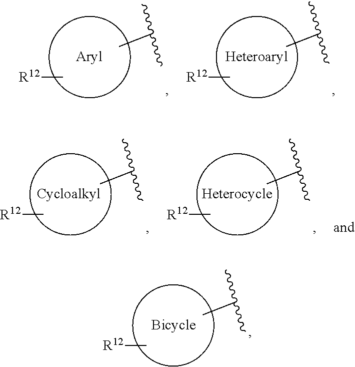

each of which is optionally substituted with 1 or 2 substituents independently selected from R⁵;
R¹⁷ is selected from:

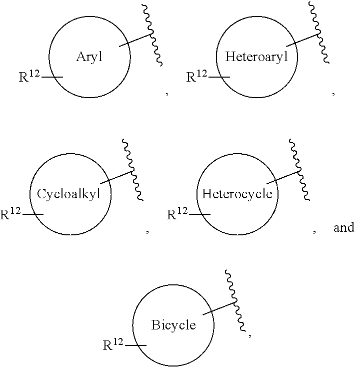

each of which is optionally substituted with 1 or 2 substituents independently selected from R⁵;
R^{17A} is selected from:

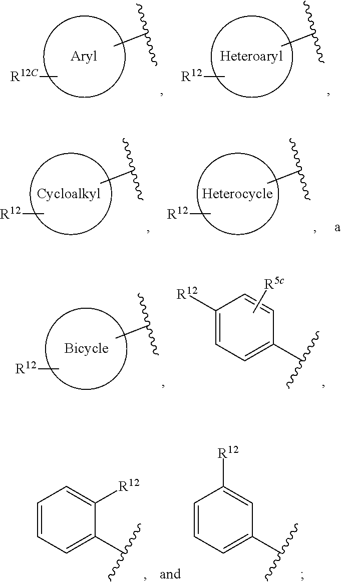

each of which is optionally substituted with 1 or 2 substituents independently selected from R⁵;
R^{18A} is selected from:

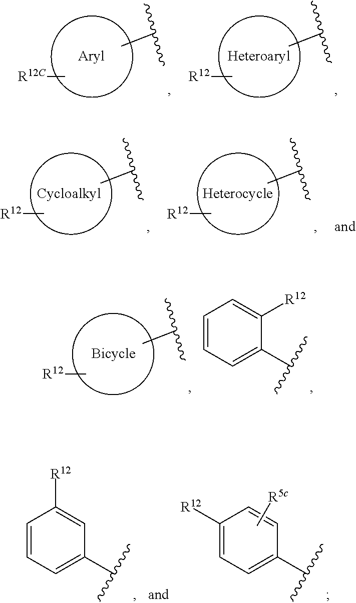

each of which is attached to the azaglutarimide moiety through a C—N bond and each of which R^{18A} is optionally substituted with 1 or 2 substituents independently selected from R⁵; for example

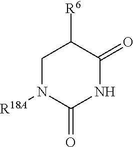

includes

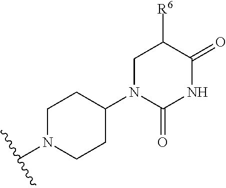

but does not include

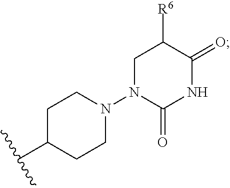

Cycle is a fused aryl or heteroaryl group optionally substituted with 1 or 2 substituents independently selected from R⁵ and substituted with one R¹² substituent;
Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a};
Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1 or 2 substituents independently selected from R^{5b};
R¹² is selected from

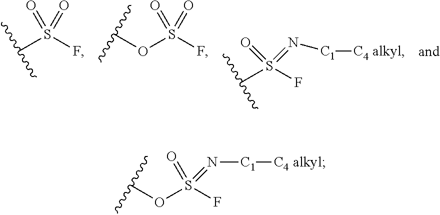

R^{12B} is selected from

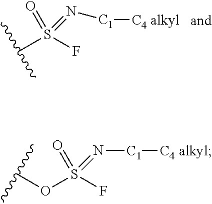

R^{12C} is selected from

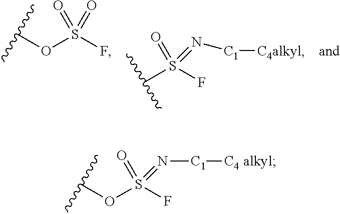

R⁷ and R⁸ at each instance are independently selected from hydrogen, alkyl, haloalkyl, alkenyl, alkynyl, aryl, heteroaryl, heterocycle, and C(O)R¹⁴; each of which except hydrogen is optionally substituted with 1 or 2 substituents independently selected from R^{10b}
in certain embodiments each R⁷ and R⁸ are selected from hydrogen and CH₃;
each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R^{10a}, and R^{10b} is independently selected from hydrogen, alkyl, haloalkyl, alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂;
R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R¹⁵;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; and
each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂.

In certain embodiments the Degron as described herein can be used alone (i.e., not as part of a Degrader) as an in vivo binder of cereblon, which can be administered to a host, for example, a human, in need thereof, in an effective amount, optionally as a pharmaceutically acceptable salt, and optionally in a pharmaceutically acceptable composition, for any therapeutic indication which can be treated by modulating the function or activity of the cereblon-containing E3 ubiquitin ligase protein complex, including but not limited to uses known for the cereblon binders thalidomide, pomalidomide, and lenalidomide. The binding of a Degron described herein to cereblon can induce a change in the protein confirmation of cereblon that allows for the degradation of a Target Protein. In certain aspects a Degron described herein is a “molecular glue” that causes the targeted degradation of a Target Protein, for example, a protein with a C2H2 zinc finger degron motif.

Non-limiting examples of proteins that may be independently selected for degradation by a Degron include ARID2, CDK1, CDK12, CDK13, CK1alpha, CSNK1A1, Cyclin K, E4F1, FAM83F, GSPT1, GSPT2, GZF1, IKZF1, IKZF2, IKZF3, IKZF4, ILF2, Myc, ODC1, p63, PDE6D, AB28, RARalpha-ZBTB16, RBM23, RBM39, RBM39, RNF166, SALL4, WBP4, ZBTB16, ZBTB16-RARalpha, ZBTB39, ZFP91, ZFP91, ZFP91, ZMYM2-FGFR1, ZMYM2-FLT3, ZNF198, ZNF276, ZNF276, ZNF517, ZNF582, ZNF653, ZNF654, ZNF692, ZNF787, ZNF827, and ZNF98. In certain embodiments the Target Protein is ARID2, aromatase, b-catenin, CDK12, NRF2, PDE6D, CK1alpha, cyclin K, GSPT1, FAM83, ILF2, ZBTB16, or ZMYM2.

Non-limiting examples of disorders which may be treated with a selected Degron described herein include abnormal cell proliferation, including a tumor or cancer, or a myelo- or lymphoproliferative disorder such as B- or T-cell lymphomas, multiple myeloma, Waldenstrom's macroglobulinemia, Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, reactive arthritis, lupus, or type I diabetes; a disease of cardiologic malfunction including hypercholesterolemia; and inflammatory conditions including asthma, chronic peptic ulcers, tuberculosis, rheumatoid arthritis, periodontitis, ulcerative colitis, and Crohn's disease.

In certain embodiments a Degron described herein is used to degrade a protein that mediates multiple myeloma, colorectal cancer, Hodgkin's lymphoma, or Non-Hodgkin's lymphoma.

In other aspects the Degron has a Tail moiety. For example, a Degron of Formula:

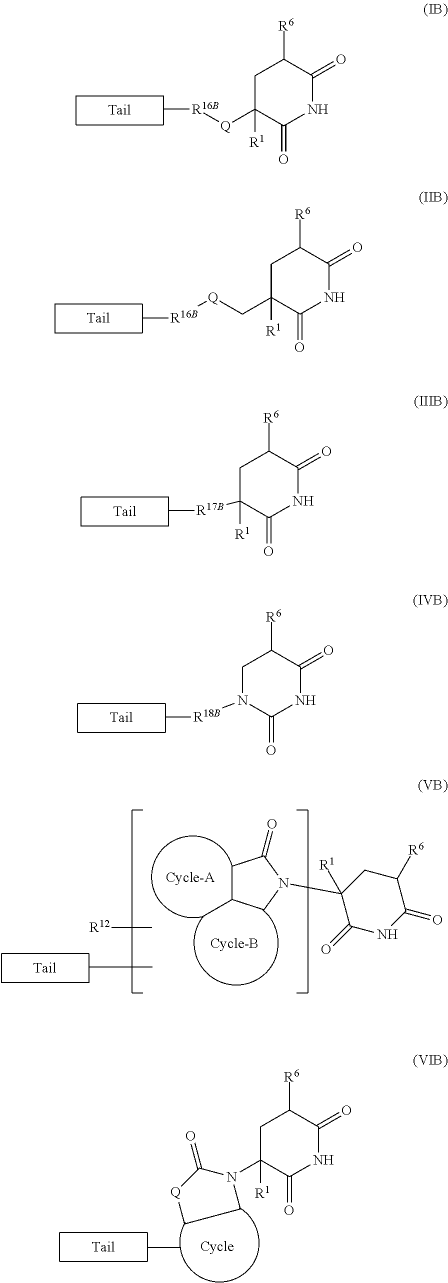

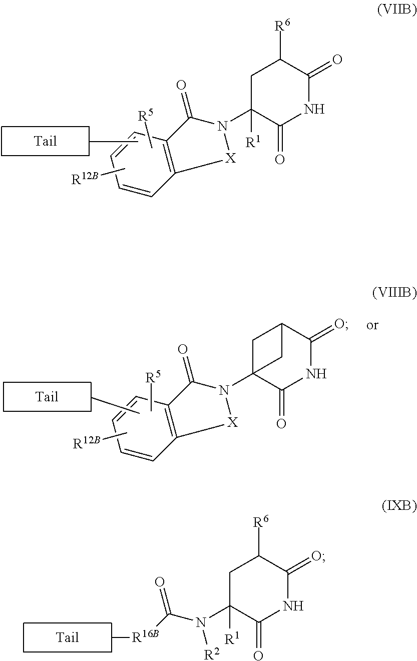

or a pharmaceutically acceptable salt thereof;
wherein:
   Tail is selected from

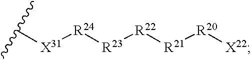

wherein
   X³¹ is selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—; each of which heterocycle, aryl, heteroaryl, and bicycle is substituted with 1, 2, or 3 substituents independently selected from R⁴⁰;
   X²²is selected from alkyl, haloalkyl, alkenyl, alkynyl, halogen, heteroaryl, heterocycle, —NR⁷R⁸, —OR⁷, —SR⁷, —C(O)R⁹, —C(S)R⁹, —S(O)₂R⁹, —OC(O)R⁹, —OS(O)R⁹, —OS(O)₂R⁹, —SC(O)R⁹, —NR⁷C(O)R⁹, —NR⁷C(S)R⁹, —NR⁷S(O)R⁹, —NR⁷S(O)₂R⁹, —P(O)(R⁹)₂, —SP(O)(R⁹)₂, —NR⁷P(O)(R⁹)₂, and —OP(O)(R⁹)₂;
   in an alternative embodiment X²² is a functional group that can be used to covalently link a Targeting Ligand to the Tail;
   R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkenyl, alkynyl, haloalkyl, alkoxy, aryl, heterocycle, heteroaryl, and carbocycle; each of which is optionally substituted with 1, 2, or 3 substituents independently selected from R⁴⁰;
   R²⁶ is independently at each occurrence selected from the group consisting of hydrogen, alkyl, arylalkyl, heteroarylalkyl, alkenyl, alkynyl, aryl, heteroaryl, and heterocycle;
   R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkenyl, and alkynyl;
   R⁴⁰ is independently at each occurrence selected from the group consisting of hydrogen, alkyl, alkenyl, alkynyl, fluoro, bromo, chloro, azide, OR⁷, NR⁷R⁸, S(O)NR⁷R⁸, S(O)2NR⁷R⁸, S(O)OR⁷, S(O)₂OR⁷, haloalkyl, aryl, heteroaryl, heterocycle, and cycloalkyl;

R⁴¹ is independently at each occurrence selected from the group consisting of hydrogen, alkyl, alkenyl, alkynyl, haloalkyl, aryl, heteroaryl, heterocycle, and cycloalkyl;
R^{16B} is selected from:

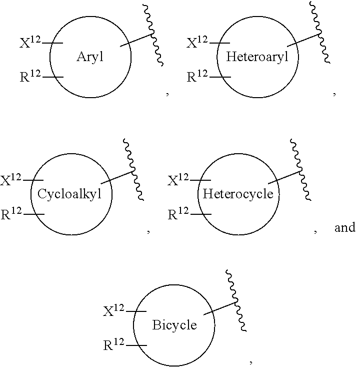

each of which is optionally substituted with 1 or 2 substituents independently selected from R⁵;
R^{17B} is selected from:

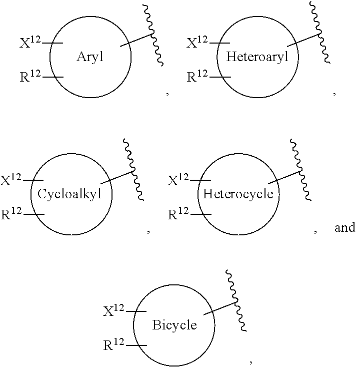

each of which is optionally substituted with 1 or 2 substituents independently selected from R⁵;
R^{18B} is selected from:

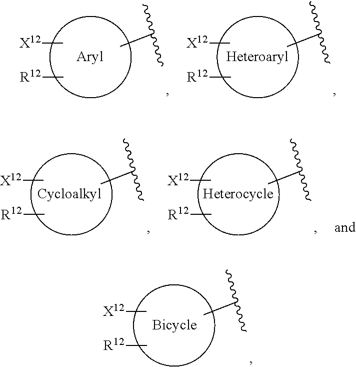

each of which is attached to the azaglutarimide moiety through a C—N bond and each of which R^{18B} is optionally substituted with 1 or 2 substituents independently selected from R⁵; and
X¹² is the attachment point to Tail or Linker.

In certain aspects, a Degrader compound of Formula IC, Formula IIC, Formula IIIC, Formula IVC, Formula VC, Formula VIC, Formula VIIC, or Formula VIIIC is provided:

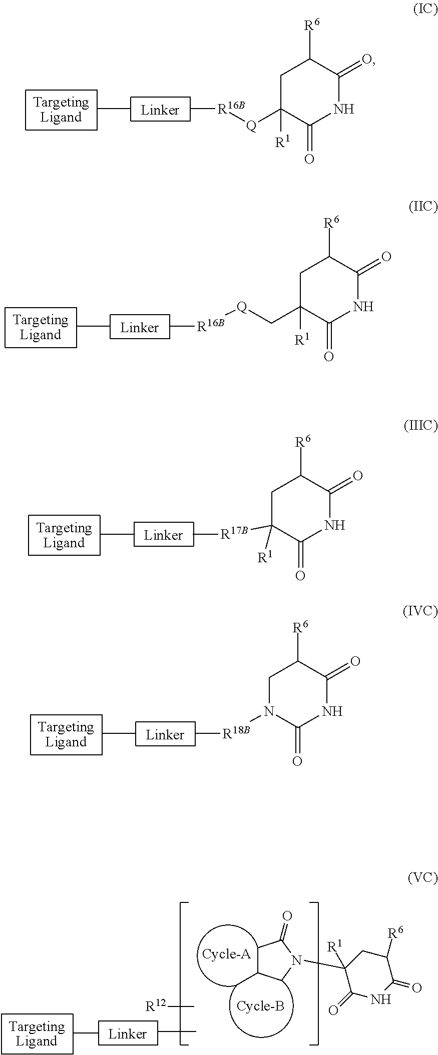

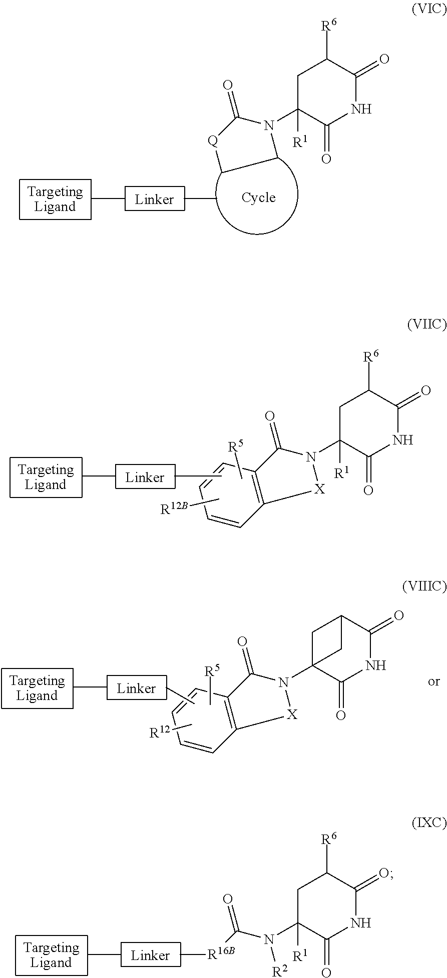

or a pharmaceutically acceptable salt thereof,
   wherein:
   Targeting Ligand is a moiety that binds to a Target Protein;
   in certain aspects Targeting Ligand is a means to bind a Target Protein;
   Target Protein is a selected protein that causes or contributes to a disease; and
   Linker is a bivalent linking group;
   and wherein all other variables are as defined herein.

In certain aspects Linker is of Formula:

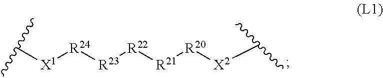

wherein:
   Xⁱ and X² are independently at each occurrence selected from bond, carbamate, heterocycle, NR⁴², C(R⁴²)₂, O, C(O), and S;
   R⁴² is independently at each occurrence selected from the group consisting of hydrogen, alkyl, heteroalkyl, heterocycle, aryl, heteroaryl, —C(O)H, —C(O)OH, —C(O)alkyl, —C(O)Oalkyl, —C(O)(aryl, heteroalkyl or heteroaryl), —C(O)O(aryl, heteroalkyl, or heteroaryl), alkenyl, and alkynyl;
   and all other variables are as defined herein.

A Degrader provided herein or its pharmaceutically acceptable salt or its pharmaceutically acceptable composition can be used to treat a disorder which is mediated by the selected Target Protein that binds to the Targeting Ligand. Therefore, in some embodiments a method to treat a host with a disorder mediated by the Target Protein is provided that includes administering an effective amount of the Degrader or its pharmaceutically acceptable salt described herein to the host, typically a human, optionally in a pharmaceutically acceptable composition.

In certain embodiments, the selected Target Protein is derived from a gene that has undergone an amplification, translocation, rearrangement, a copy number variation, alteration, deletion, mutation, or inversion event which causes or is caused by a medical disorder. In certain aspects, the selected Target Protein has been post-translationally modified by one, or combinations, of phosphorylation, acetylation, acylation including propionylation and crotylation, N-linked glycosylation, amidation, hydroxylation, methylation, poly-methylation, O-linked glycosylation, pyroglutamoylation, myristoylation, farnesylation, geranylation, sumoylation, or sulfation which causes or is caused by a medical disorder.

One non-limiting example of a disorder treatable by such compounds is abnormal cellular proliferation, such as a tumor or cancer, wherein the Target Protein is an oncogenic protein or a signaling mediator of an abnormal cellular proliferative pathway and its degradation decreases abnormal cell growth.

Compounds and methods are presented for the treatment of a patient with a disorder mediated by a protein that is targeted for selective degradation that includes administering an effective amount of a Degron or Degrader of the present invention described herein to a patient in need thereof, optionally in a pharmaceutically acceptable carrier (composition). In typical embodiments, the patient is a human.

In certain embodiments, the disorder is selected from a neoplasm, tumor, cancer, abnormal cellular proliferation, immune disorder, inflammatory disorder, graft-versus-host rejection, viral infection, bacterial infection, an amyloid-based proteinopathy, a proteinopathy, or fibrotic disorder.

In certain aspects, the Target Protein is a protein that is not druggable in the classic sense in that it does not have a binding pocket or an active site that can be inhibited or otherwise 5 bound and cannot be easily allosterically controlled. In other aspects, the Target Protein is a protein that is druggable in the classic sense.

In certain embodiments, the Degron or Degrader compound has at least one desired isotopic substitution of an atom, at an amount above the natural abundance of the isotope, i. e., enriched.

In one embodiment, the Degron or Degrader compound includes a deuterium or multiple deuterium atoms.

Other features and advantages of the present application will be apparent from the following detailed description and claims.

The present invention therefore includes at least the following features:
(a) A Degron compound as described herein, or a pharmaceutically acceptable salt, isotopic derivative, or prodrug thereof,
(b) The use of a Degron compound or a pharmaceutically acceptable salt thereof, isotopic derivative or prodrug thereof as described herein as a molecular glue to bind to cereblon, resulting in the degradation of a Target Protein;
(c) A Degron with a Tail, or a pharmaceutically acceptable salt, isotopic derivative or prodrug thereof or a therapeutic use thereof,
(d) A Degrader compound as described herein, or a pharmaceutically acceptable salt, isotopic derivative or prodrug thereof;
(e) A Degrader compound or a pharmaceutically acceptable salt, isotopic derivative, or prodrug thereof for the treatment of a disorder that is mediated by a Target Protein, wherein the compound includes a Targeting Ligand for the Target Protein;
(f) Use of a Degron compound in an effective amount in the treatment of a human patient, with a disorder that responds to such treatment, including by altering the cereblon-based ubiquitination of a protein, such as for example, abnormal cellular proliferation such as a tumor or cancer, an immune or autoimmune or inflammatory disorder, a cardiologic disorder, an infectious disease, or other disorder that responds to such treatment;
(g) Use of a Degrader compound in an effective amount in the treatment of a patient, typically a human, with a disorder mediated by a Target Protein, such as for example, abnormal cellular proliferation such as a tumor or cancer, an immune or autoimmune or inflammatory disorder, a cardiologic disorder, an infectious disease, or other disorder that responds to such treatment;
(h) Use of a Degron or Degrader or a pharmaceutically acceptable salt, isotopic derivative, or prodrug thereof in the manufacture of a medicament for the treatment of a medical disorder;
(i) A method for manufacturing a medicament intended for the therapeutic treatment of a disorder in a host characterized in that a Degron or Degrader is used in the manufacture;
(j) A pharmaceutical formulation comprising a Degron or Degrader compound or a pharmaceutically acceptable salt, isotopic derivative, or prodrug thereof with a pharmaceutically acceptable carrier or diluent;
(k) A Degron or Degrader compound as described herein as a mixture of enantiomers or diastereomers (as relevant), including as a racemate;
(l) A Degron or Degrader compound as described herein in enantiomerically or diastereomerically (as relevant) enriched form, including an isolated enantiomer or diastereomer (i.e., greater than 85, 90, 95, 97, or 99% pure); and
(m) A process for the preparation of therapeutic products that contain an effective amount of a Degron or Degrader compound or a pharmaceutically acceptable salt, isotopic derivative, or prodrug thereof optionally with a pharmaceutically acceptable carrier or diluent.

### BRIEF DESCRIPTION OF THE FIGURES

FIG. 1A-1C provide non-limiting examples of Retinoid X Receptor (RXR) Targeting Ligands wherein R represents exemplary points at which the Linker can be attached.

FIG. 1D-1F provide non-limiting examples of general Dihydrofolate reductase (DHFR) Targeting Ligands wherein R represents exemplary points at which the Linker can be attached.

FIG. 1G provides non-limiting examples of Bacillus anthracis Dihydrofolate reductase (BaDHFR) Targeting Ligands wherein R represents exemplary points at which the Linker can be attached.

FIG. 1H-1J provide non-limiting examples of Heat Shock Protein 90 (HSP90) Targeting Ligands wherein R represents exemplary points at which the Linker can be attached.

FIG. 1K-1Q provide non-limiting examples of General Kinase and Phosphatase Targeting Ligands wherein R represents exemplary points at which the Linker can be attached.

FIG. 1R-1S provides non-limiting examples of Tyrosine Kinase Targeting Ligands wherein R represents exemplary points at which the Linker can be attached.

FIG. 1T provides non-limiting examples of Aurora Kinase Targeting Ligands wherein R represents exemplary points at which the Linker can be attached.

FIG. 1U provides non-limiting examples of Protein Tyrosine Phosphatase Targeting Ligands wherein R represents exemplary points at which the Linker can be attached.

FIG. 1V provides non-limiting examples of ALK Targeting Ligands wherein R represents exemplary points at which the Linker can be attached.

FIG. 1W provides non-limiting examples of ABL Targeting Ligands wherein R represents exemplary points at which the Linker can be attached.

FIG. 1X provides non-limiting examples of JAK2 Targeting Ligands wherein R represents exemplary points at which the Linker can be attached.

FIG. 1Y-1Z provide non-limiting examples of MET Targeting Ligands wherein R represents exemplary points at which the Linker can be attached.

FIG. 1AA provides non-limiting examples of mTORC1 and/or mTORC2 Targeting Ligands wherein R represents exemplary points at which the Linker can be attached.

FIG. 1BB-1CC provide non-limiting examples of Mast/stem cell growth factor receptor (SCFR), also known as c-KIT receptor, Targeting Ligands wherein R represents exemplary points at which the Linker can be attached.

FIG. 1DD provides non-limiting examples of IGF1R and/or IR Targeting Ligands wherein R represents exemplary points at which the Linker can be attached.

FIG. 1EE-1FF provide non-limiting examples of HDM2 and/or MDM2 Targeting Ligands wherein R represents exemplary points at which the Linker can be attached.

FIG. 1GG-1MM provide non-limiting examples of BET Bromodomain-Containing Protein Targeting Ligands wherein R represents exemplary points at which the Linker can be attached.

FIG. 1NN provides non-limiting examples of HDAC Targeting Ligands wherein R represents exemplary points at which the Linker can be attached.

FIG. 1OO provides non-limiting examples of RAF Receptor Targeting Ligands wherein R represents exemplary points at which the Linker can be attached.

FIG. 1PP provides non-limiting examples of FKBP Receptor Targeting Ligands wherein R represents exemplary points at which the Linker can be attached.

FIG. 1QQ-1TT provide non-limiting examples of Androgen Receptor Targeting Ligands wherein R represents exemplary points at which the Linker can be attached.

FIG. 1UU provides non-limiting examples of Estrogen Receptor Targeting Ligands wherein R represents exemplary points at which the Linker can be attached.

FIG. 1VV-1WW provide non-limiting examples of Thyroid Hormone Receptor Targeting Ligands wherein R represents exemplary points at which the Linker can be attached.

FIG. 1XX provides non-limiting examples of HIV Protease Targeting Ligands wherein R represents exemplary points at which the Linker can be attached.

FIG. 1YY provides non-limiting examples of HIV Integrase Targeting Ligands wherein R represents exemplary points at which the Linker can be attached.

FIG. 1ZZ provides non-limiting examples of HCV Protease Targeting Ligands wherein R represents exemplary points at which the Linker can be attached.

FIG. 1AAA provides non-limited examples of AP1 and/or AP2 Targeting Ligands wherein R represents exemplary points at which the Linker can be attached.

FIG. 1BBB-1CCC provide non-limiting examples of MCL-1 Targeting Ligands wherein R represents exemplary points at which the Linker can be attached.

FIG. 1DDD provides non-limiting examples of IDH1 Targeting Ligands wherein R represents exemplary points at which the Linker can be attached.

FIG. 1EEE-1FFF provide non-limiting examples of RAS or RASK Targeting Ligands wherein R represents exemplary points at which the Linker can be attached.

FIG. 1GGG provides non-limiting examples of MERTK or MER Targeting Ligands 20 wherein R represents exemplary points at which the Linker can be attached.

FIG. 1HHH-1III provide non-limiting examples of EGFR Targeting Ligands wherein R represents exemplary points at which the Linker can be attached.

FIG. 1JJJ-1KKK provide non-limiting examples of FLT3 Targeting Ligands wherein R represents exemplary points at which the Linker can be attached.

FIG. 1LLL provides non-limiting examples of SMARCA2 Targeting Ligands wherein R represents exemplary points at which the Linker can be attached.

FIG. 2A provides non-limiting examples of the kinase inhibitor Targeting Ligands U09-CX-5279 (derivatized) wherein R represents exemplary points at which the Linker can be attached.

FIG. 2B-2C provide non-limiting examples of kinase inhibitor Targeting Ligands, including the kinase inhibitor compounds Y1W and Y1X (derivatized) wherein R represents exemplary points at which the Linker can be attached. For additional examples and related ligands, see, the kinase inhibitors identified in Millan et al. “Design and Synthesis of Inhaled P38 Inhibitors for the Treatment of Chronic Obstructive Pulmonary Disease” J. Med. Chem., 54: 7797 (2011).

FIG. 2D provides non-limiting examples of kinase inhibitor Targeting Ligands, including the kinase inhibitor compounds 6TP and 0TP (derivatized) wherein R represents exemplary points at which the Linker can be attached. For additional examples and related ligands, see, the kinase inhibitors identified in Schenkel et al. “Discovery of Potent and Highly Selective Thienopyridine Janus Kinase 2 Inhibitors” J. Med. Chem., 54 (24): 8440-8450 (2011).

FIG. 2E provides non-limiting examples of kinase inhibitor Targeting Ligands, including the kinase inhibitor compound 07U wherein R represents exemplary points at which the Linker can be attached. For additional examples and related ligands, see, the kinase inhibitors identified in Van Eis et al. “2 6-Naphthyridines as potent and selective inhibitors of the novel protein kinase C isozymes” Biorg. Med. Chem. Lett., 21(24): 7367-72 (2011).

FIG. 2F provides non-limiting examples of kinase inhibitor Targeting Ligands, including the kinase inhibitor compound YCF, wherein R represents exemplary points at which the Linker can be attached. For additional examples and related ligands, see, the kinase inhibitors identified in Lountos et al. “Structural Characterization of Inhibitor Complexes with Checkpoint Kinase 2 (Chk2) a Drug Target for Cancer Therapy” J. Struct. Biol., 176: 292 (2011).

FIG. 2G-2H provide non-limiting examples of kinase inhibitor Targeting Ligands, including the kinase inhibitors XK9 and NXP (derivatized) wherein R represents exemplary points at which the Linker can be attached. For additional examples and related ligands, see, the kinase inhibitors identified in Lountos et al. “Structural Characterization of Inhibitor Complexes with Checkpoint Kinase 2 (Chk2) a Drug Target for Cancer Therapy” J. Struct. Biol., 176: 292 (2011).

FIG. 2I-2J provide non-limiting examples of kinase inhibitor Targeting Ligands wherein R represents exemplary points at which the spacer r is attached.

FIG. 2K-2M provide non-limiting examples of Cyclin Dependent Kinase 9 (CDK9) Targeting Ligands wherein R represents exemplary points at which the Linker can be attached. For additional examples and related ligands, see, Baumli et al._“The structure of P-TEFb (CDK9/cyclin T1) its complex with flavopiridol and regulation by phosphorylation.” Embo J., 27: 1907-1918 (2008); Bettayeb et al._“CDK Inhibitors Roscovitine and CR8 Trigger Mcl-1 Down-Regulation and Apoptotic Cell Death in Neuroblastoma Cells.” Genes Cancer, 1: 369-380 (2010); Baumli et al._“Halogen bonds form the basis for selective P-TEFb inhibition by DRB.” Chem. Biol. 17: 931-936 (2010); Hole et al. “Comparative Structural and Functional Studies of 4-(Thiazol- 5-Yl)-2-(Phenylamino)Pyrimidine-5-Carbonitrile Cdk9 Inhibitors Suggest the Basis for Isotype Selectivity.” J. Med. Chem. 56: 660 (2013); Lucking et al. “Identification of the potent and highly selective PTEFb inhibitor BAY 1251152 for the treatment of cancer - From p.o. to i.v. application via scaffold hops.” Lücking et al. U. AACR Annual Meeting, Apr. 1-5, 2017 Washington, D.C. USA.

FIG. 2N-2P provide non-limiting examples of Cyclin Dependent Kinase 4/6 (CDK4/6) Targeting Ligands wherein R represents exemplary points at which the Linker can be attached. For additional examples and related ligands, see, Lu H._and Schulze-Gahmen U._“Toward understanding the structural basis of cyclin-dependent kinase 6 specific inhibition.” J. Med Chem., 49: 3826-3831 (2006); 4-(Pyrazol-4-yl)-pyrimidines as selective inhibitors of cyclin-dependent kinase 4/6. Cho et al. (2010) J. Med. Chem. 53: 7938-7957; Cho Y. S. et al. “Fragment-Based Discovery of 7-Azabenzimidazoles as Potent Highly Selective and Orally Active CDK4/6 Inhibitors.” ACS Med Chem Lett 3: 445-449 (2012); Li Z. et al. “Discovery of AMG 925 a FLT3 and CDK4 dual kinase inhibitor with preferential affinity for the activated state of FLT3.” J. Med Chem. 57: 3430-3449 (2014); Chen P. et al. “Spectrum and Degree of CDK Drug Interactions Predicts Clinical Performance.” Mol. Cancer Ther. 15: 2273-2281 (2016).

FIG. 2Q provides non-limiting examples of Cyclin Dependent Kinase 12 and/or Cyclin Dependent Kinase 13 Targeting Ligands wherein R represents exemplary points at which the Linker can be attached. For additional examples and related ligands, see, Zhang T. et al. “Covalent Targeting of Remote Cysteine Residues to Develop Cdk12 and Cdk13 Inhibitors.” Nat. Chem. Biol. 12: 876 (2016).

FIG. 2R-2S provide non-limiting examples of Glucocorticoid Receptor Targeting Ligands wherein R represents exemplary points at which the Linker can be attached.

FIG. 2T-2U provide non-limiting examples of RasG12C Targeting Ligands wherein R represents exemplary points at which the Linker can be attached.

FIG. 2V provides non-limiting examples of Her3 Targeting Ligands wherein R represents exemplary points at which the Linker can be attached and R¹¹ is

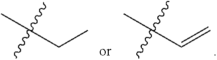

FIG. 2W provides non-limiting examples of Bcl-2 or Bcl-XL Targeting Ligands wherein R represents exemplary points at which the Linker can be attached.

FIG. 2X-2NN provide non-limiting examples of BCL2 Targeting Ligands wherein R represents exemplary points at which the Linker can be attached. For additional examples and related ligands, see, Toure B. B. et al. “The role of the acidity of N-heteroaryl sulfonamides as inhibitors of bcl-2 family protein-protein interactions.” ACS Med Chem Lett, 4: 186-190 (2013); Porter J. et al. “Tetrahydroisoquinoline Amide Substituted Phenyl Pyrazoles as Selective Bcl-2 Inhibitors” Bioorg. Med. Chem. Lett. 19: 230 (2009); Souers A. J. et al. “ABT-199 a potent and selective BCL-2 inhibitor achieves antitumor activity while sparing platelets.” Nature Med. 19: 202-208 (2013); Angelo Aguilar et al. “A Potent and Highly Efficacious Bcl-2/Bcl-xL Inhibitor” J Med Chem. 56(7): 3048-3067 (2013); Longchuan Bai et al. “BM-1197: A Novel and Specific Bcl-2/Bcl-xL Inhibitor Inducing Complete and Long-Lasting Tumor Regression In Vivo” PLoS ONE 9(6): e99404; Fariba Nematil et al. “Targeting Bcl-2/Bcl-XL Induces Antitumor Activity in Uveal Melanoma Patient-Derived Xenografts” PLoS ONE 9(1): e80836; WO2015011396 titled “Novel derivatives of indole and pyrrole method for the production thereof and pharmaceutical compositions containing same”; WO2008060569A1 titled “Compounds and methods for inhibiting the interaction of Bcl proteins with binding partners”; “Inhibitors of the anti-apoptotic Bcl-2 proteins: a patent review” Expert Opin. Ther. Patents 22(1):2008 (2012); and, Porter et al. “Tetrahydroisoquinoline amide substituted phenyl pyrazoles as selective Bcl-2 inhibitors” BioorgMed Chem Lett., 19(1):230-3 (2009).

FIG. 200-2UU provide non-limiting examples of BCL-XL Targeting Ligands wherein R represents exemplary points at which the Linker can be attached. For additional examples and related ligands, see, Zhi-Fu Tao et al. “Discovery of a Potent and Selective BCL-XL Inhibitor with in Vivo Activity” ACSMed. Chem. Lett., 5: 1088-1093 (2014); Joel D. Leverson et al. “Exploiting selective BCL-2 family inhibitors to dissect cell survival dependencies and define improved strategies for cancer therapy” Science Translational Medicine, 7:279ra40 (2015); and, the crystal structure PDB 3ZK6 (Guillaume Lessene et al. “Structure-guided design of a selective BCL-XL inhibitor” Nature Chemical Biology 9: 390-397 (2013)) FIG. 2VV provides non-limiting examples of PPAR-gamma Targeting Ligands wherein R represents exemplary points at which the Linker can be attached.

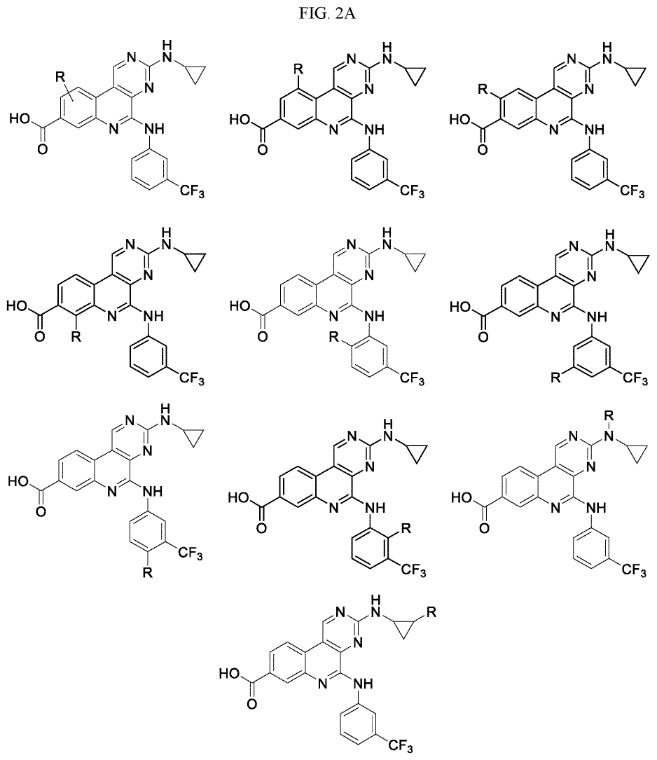

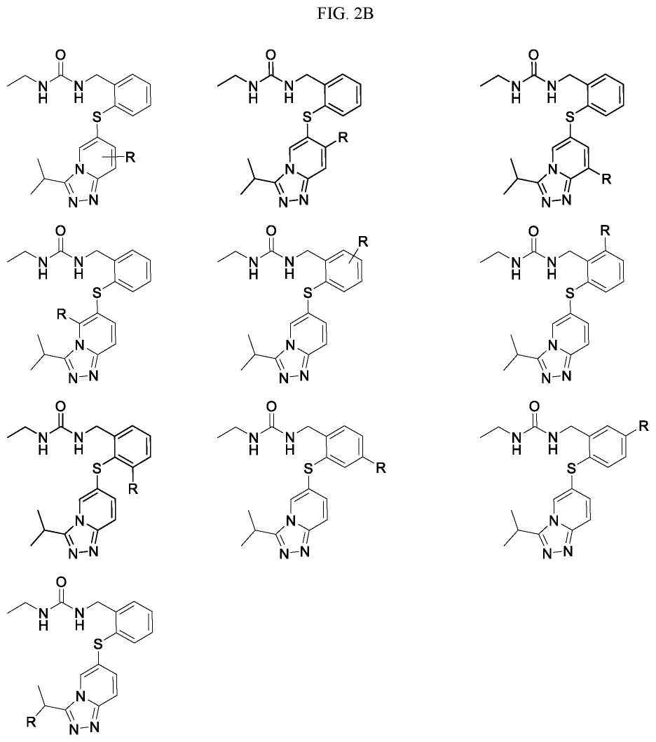

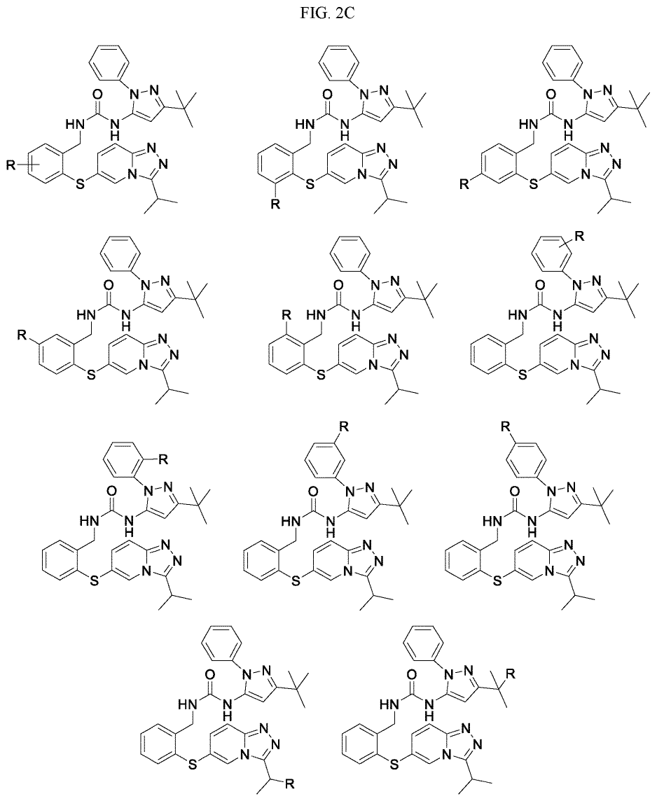

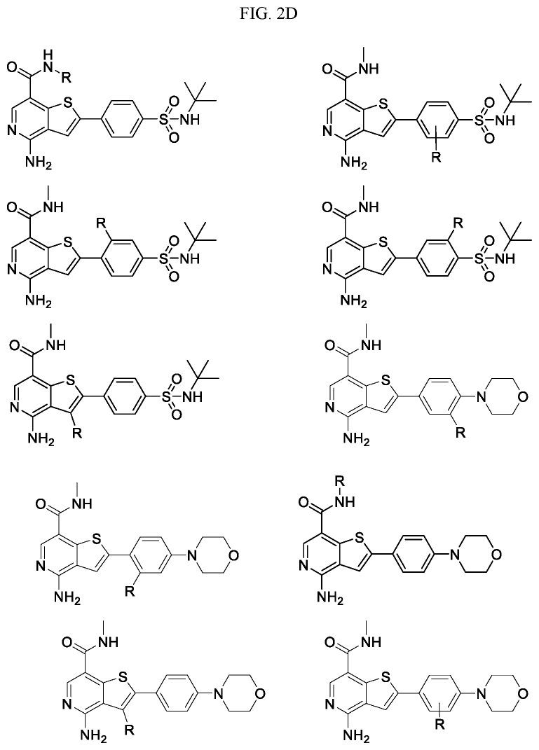

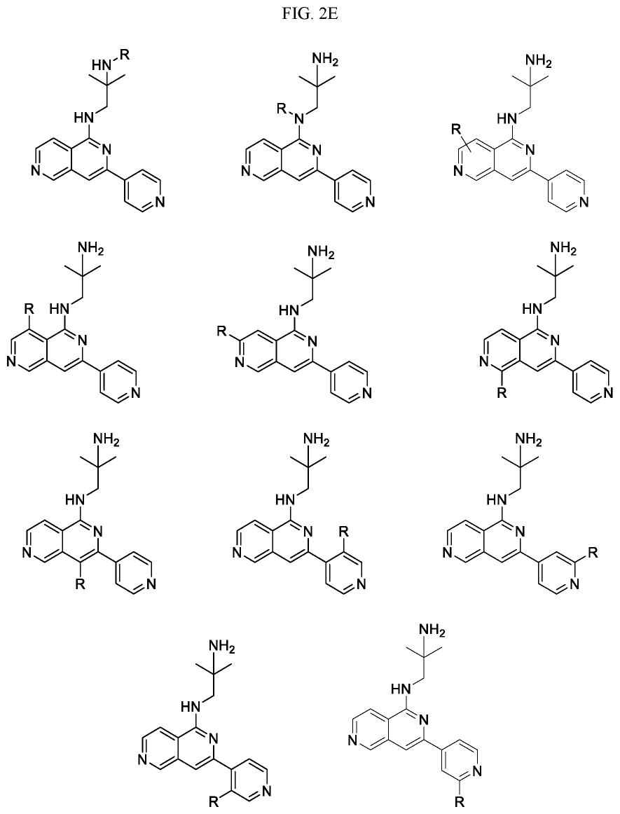

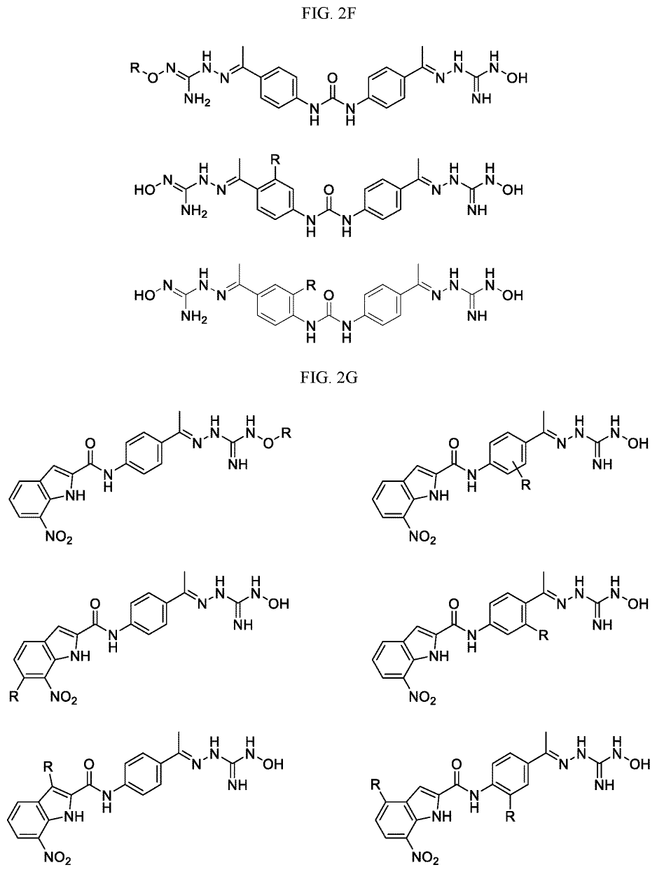

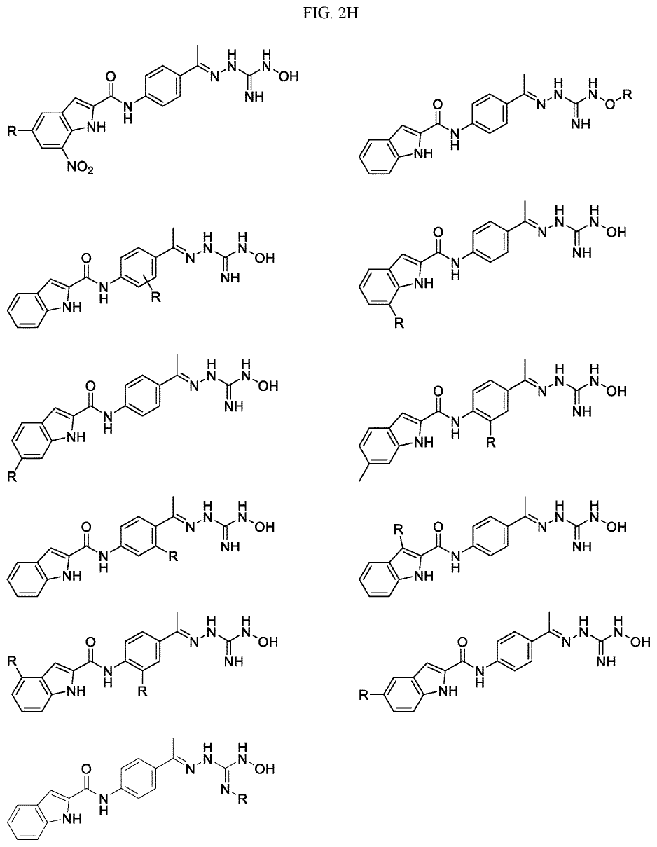

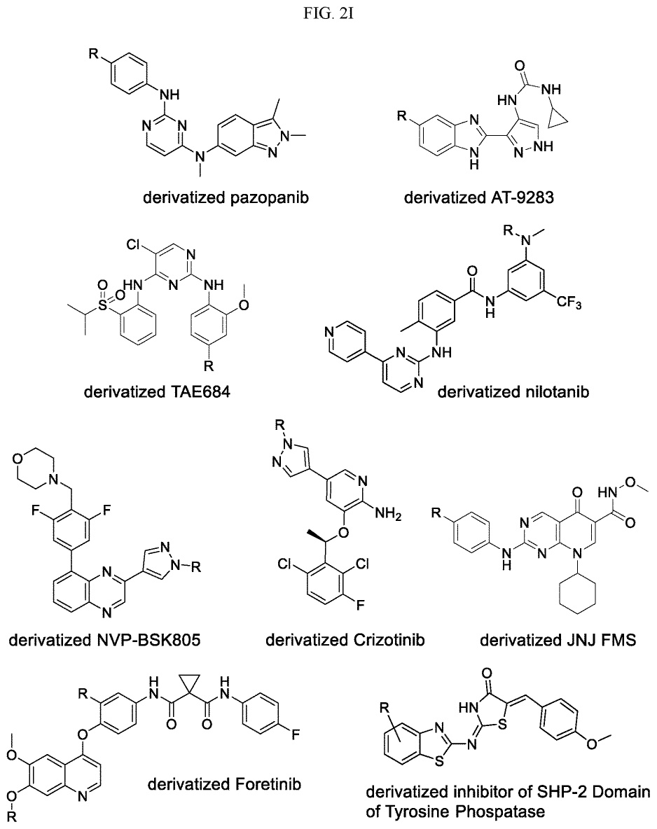

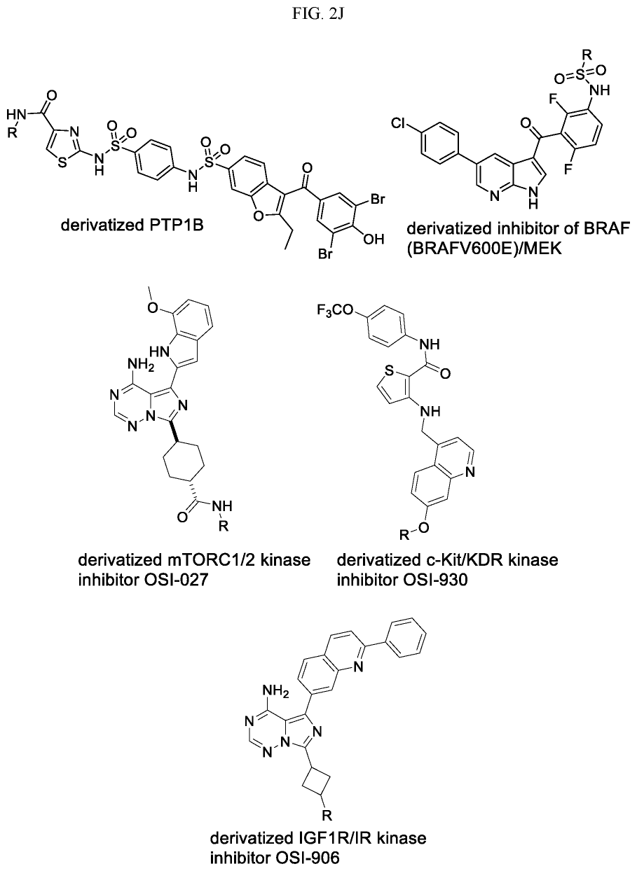

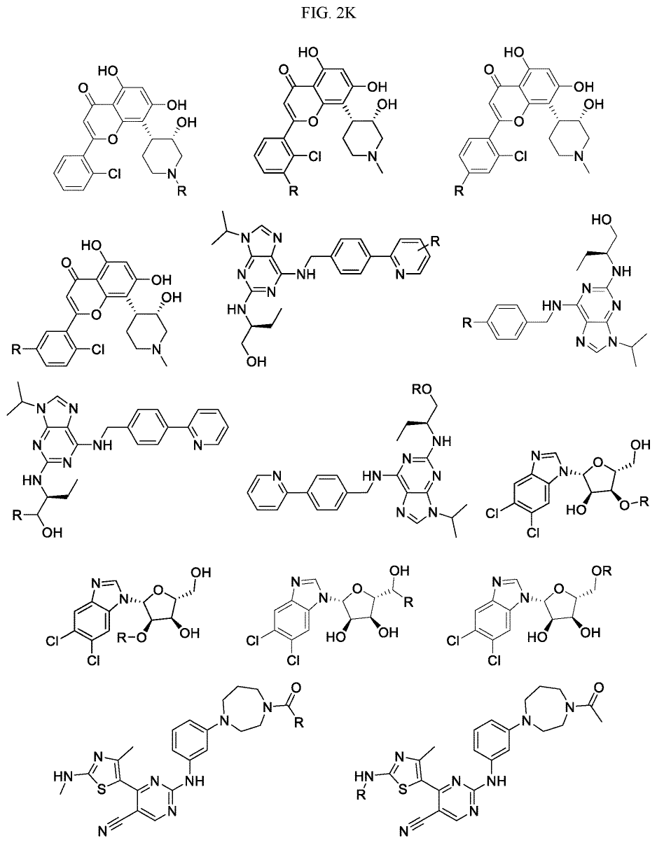

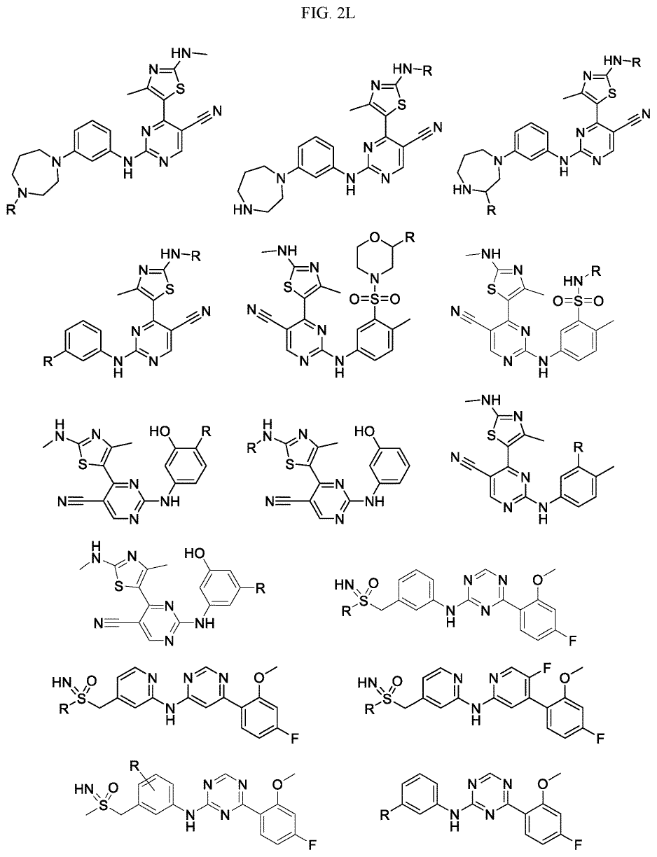

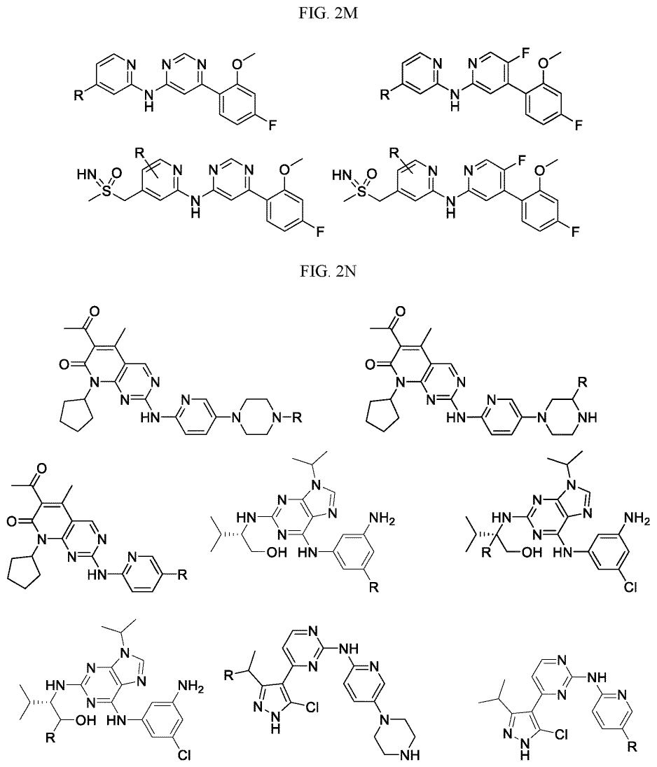

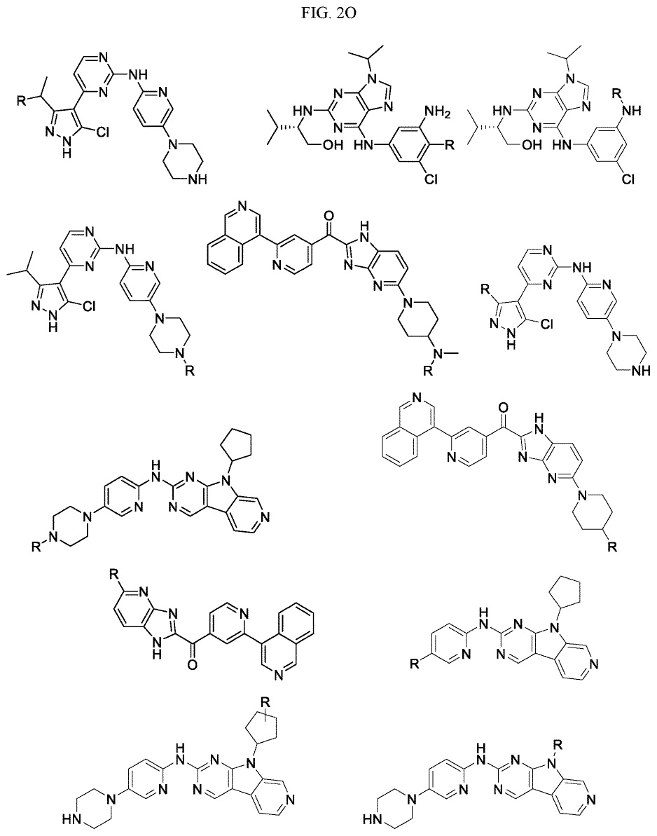

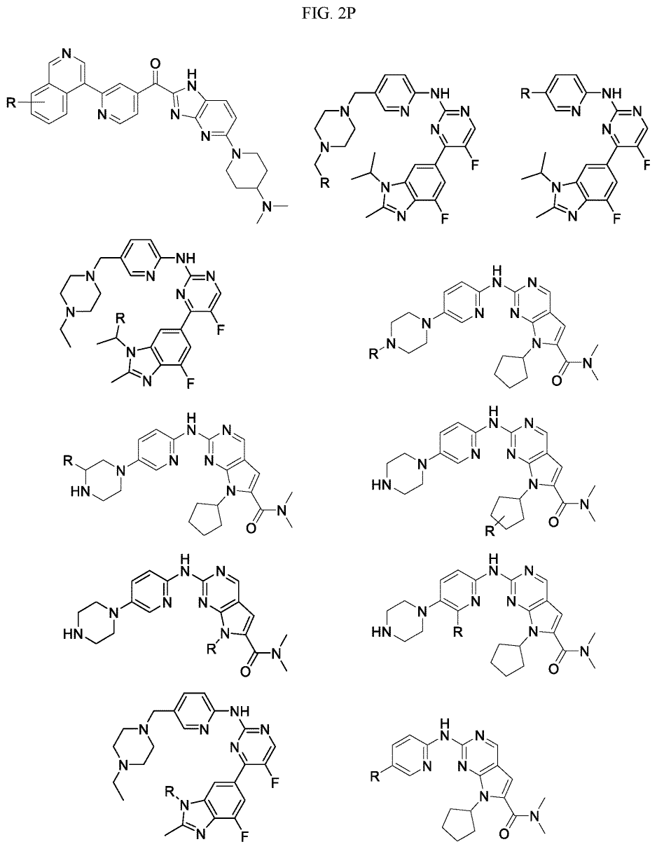

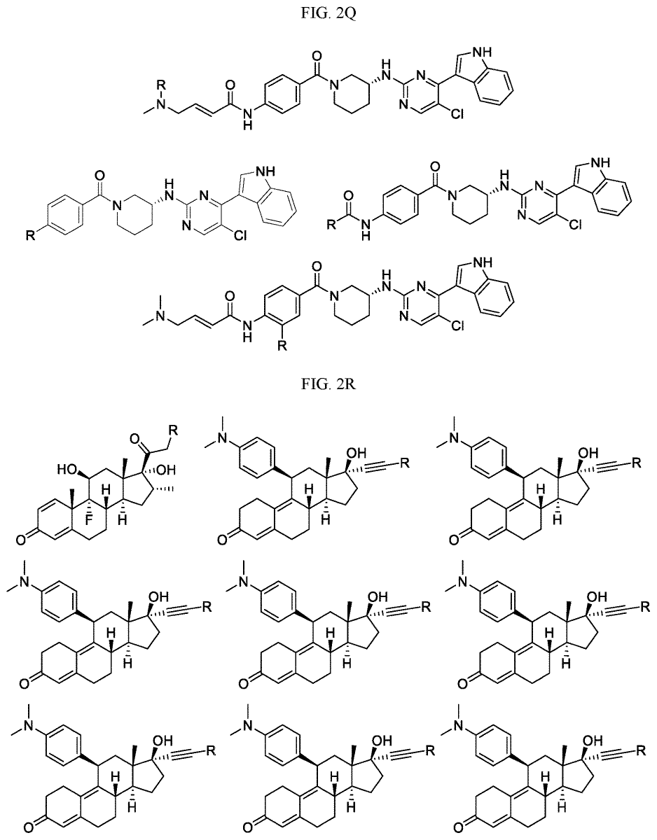

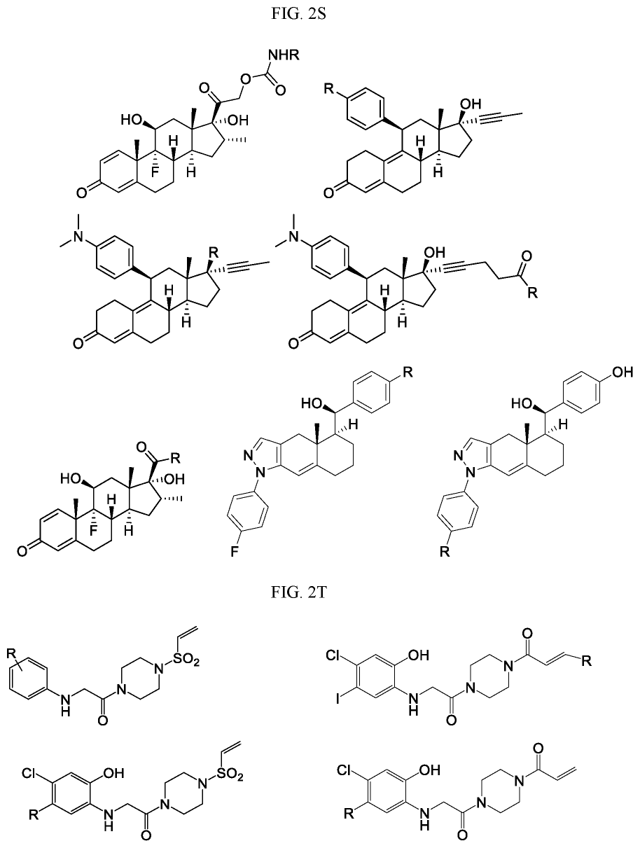

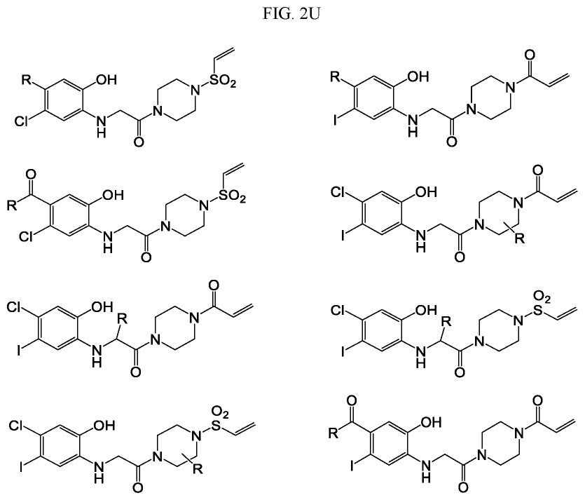

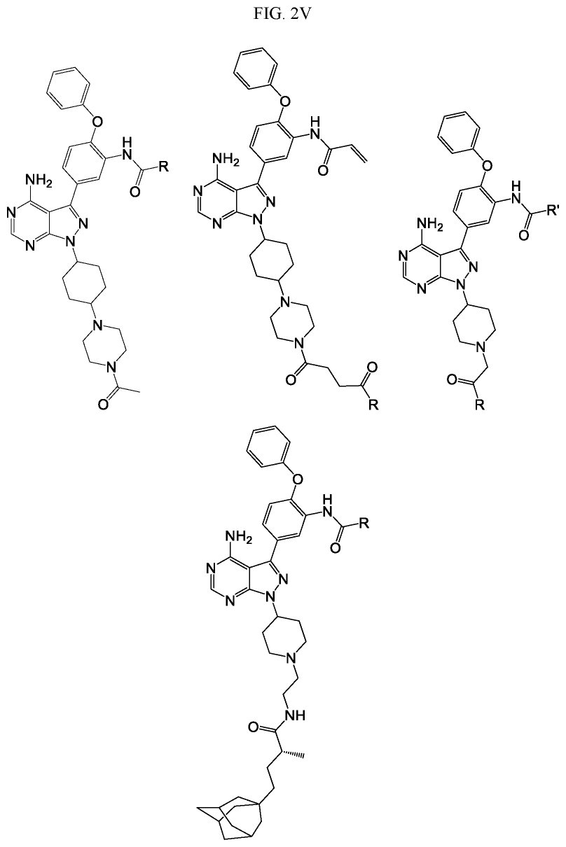

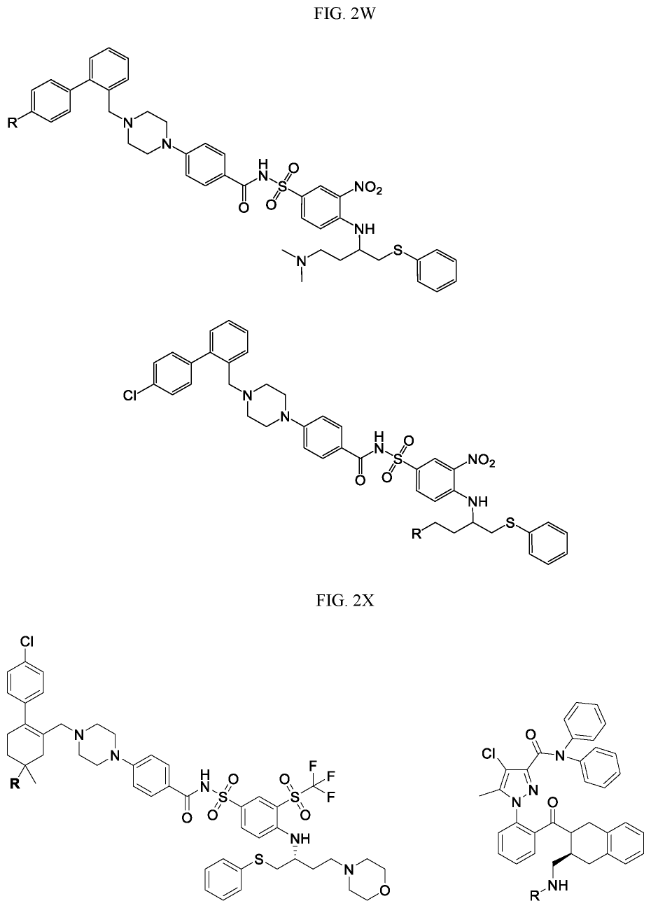

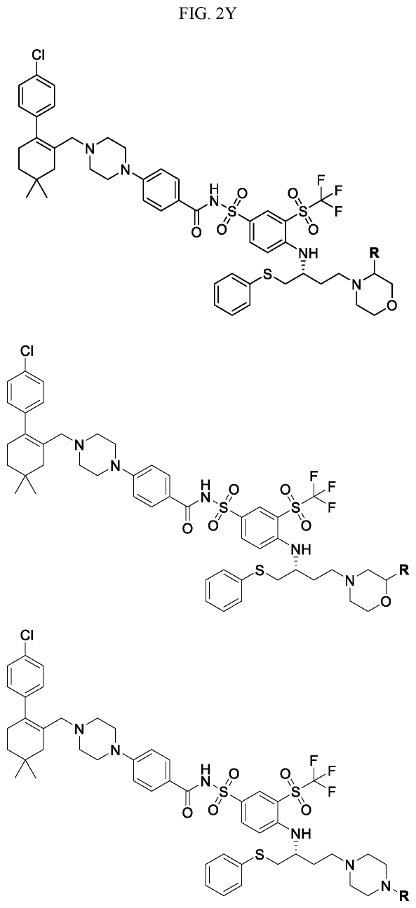

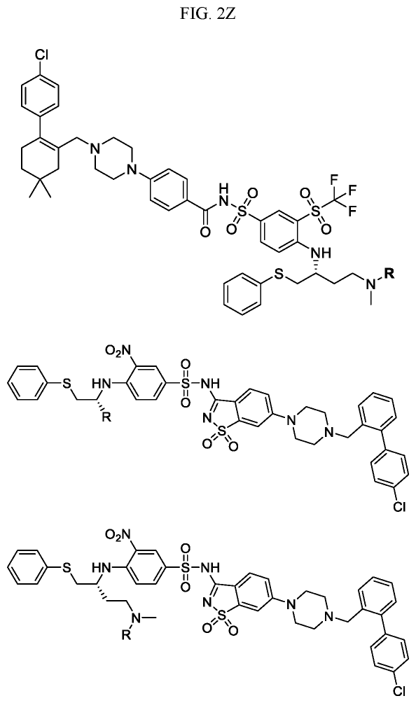

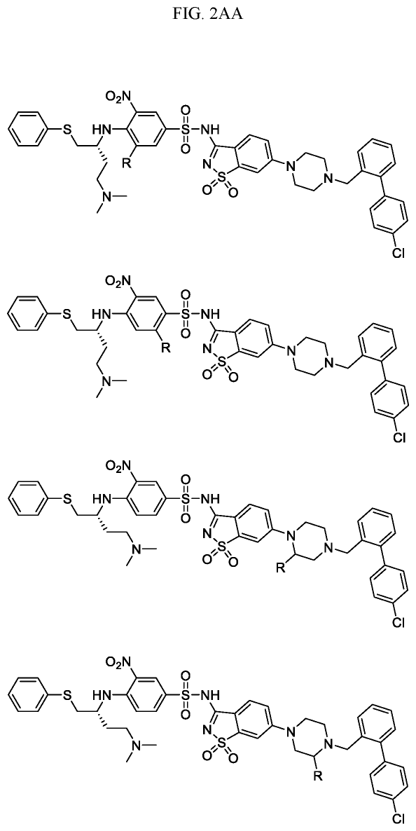

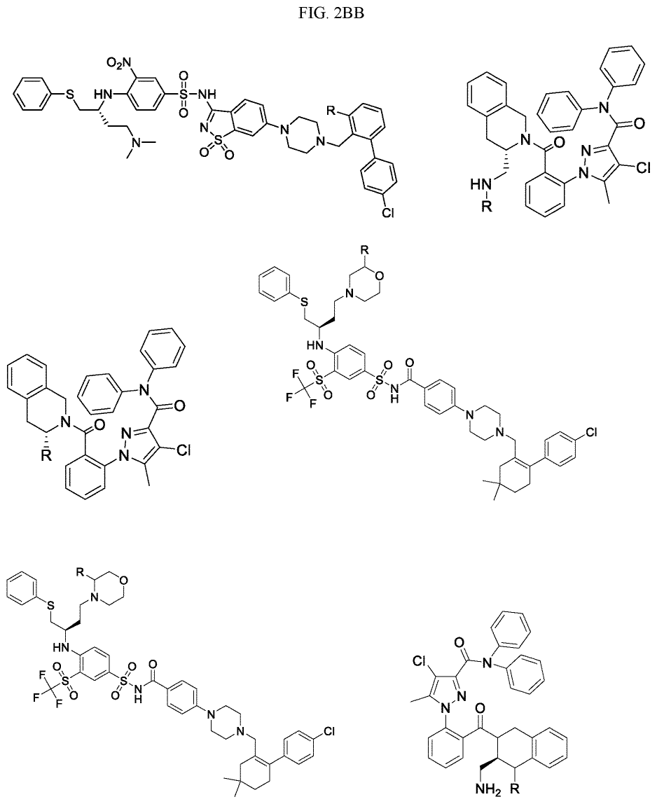

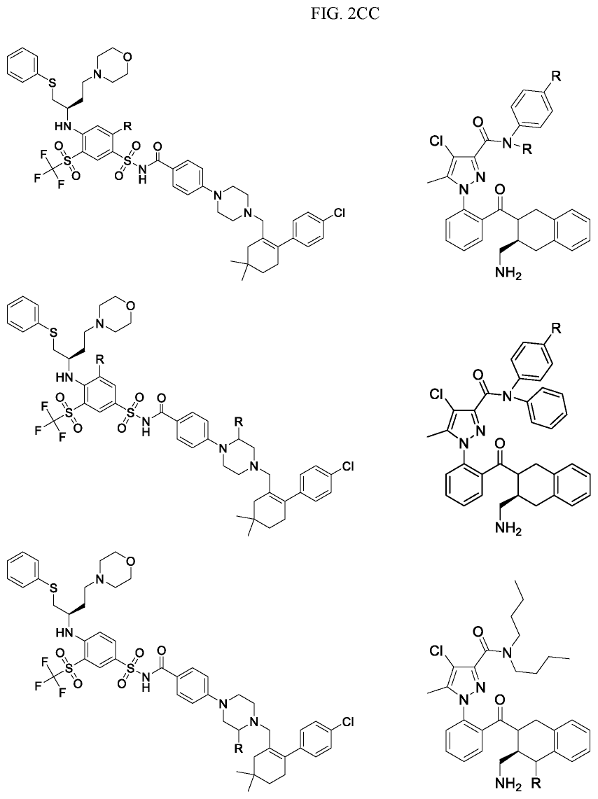

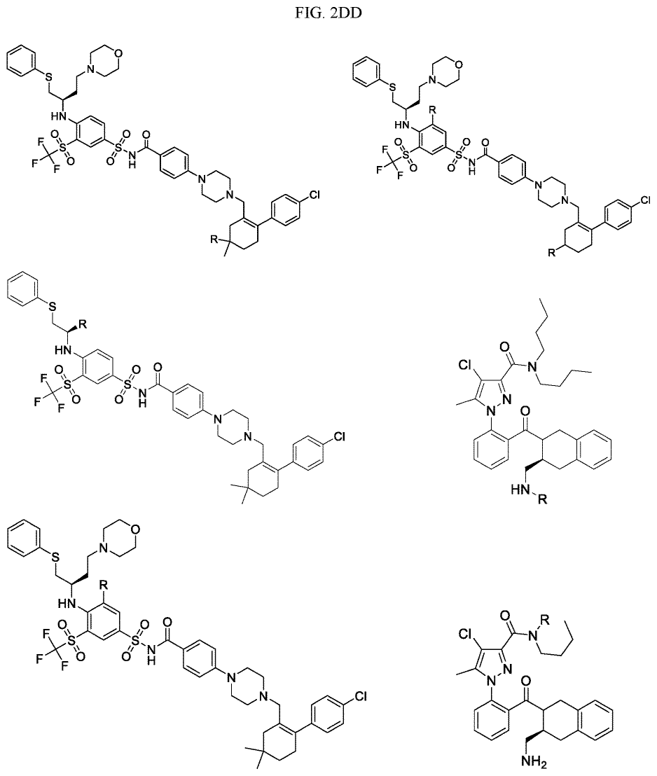

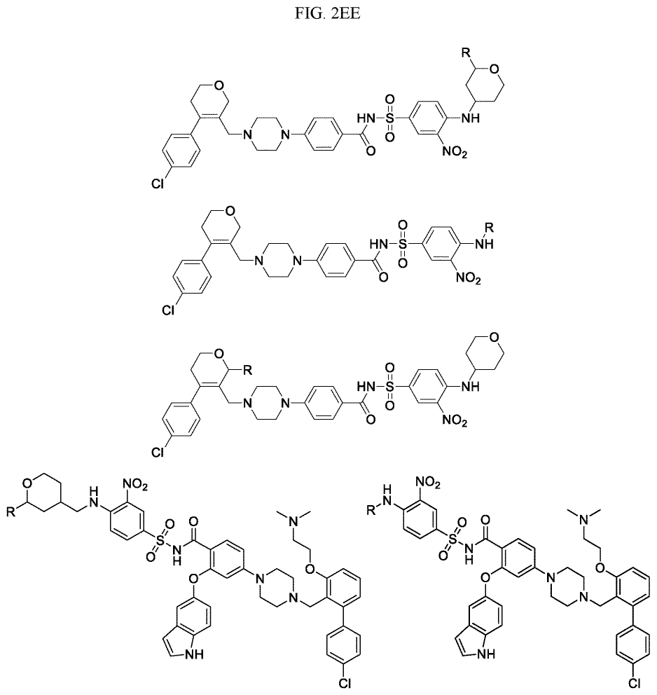

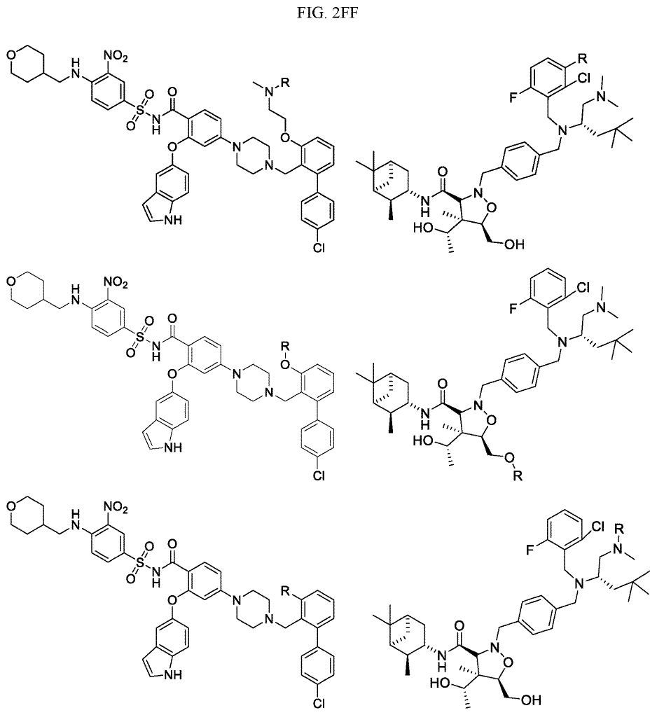

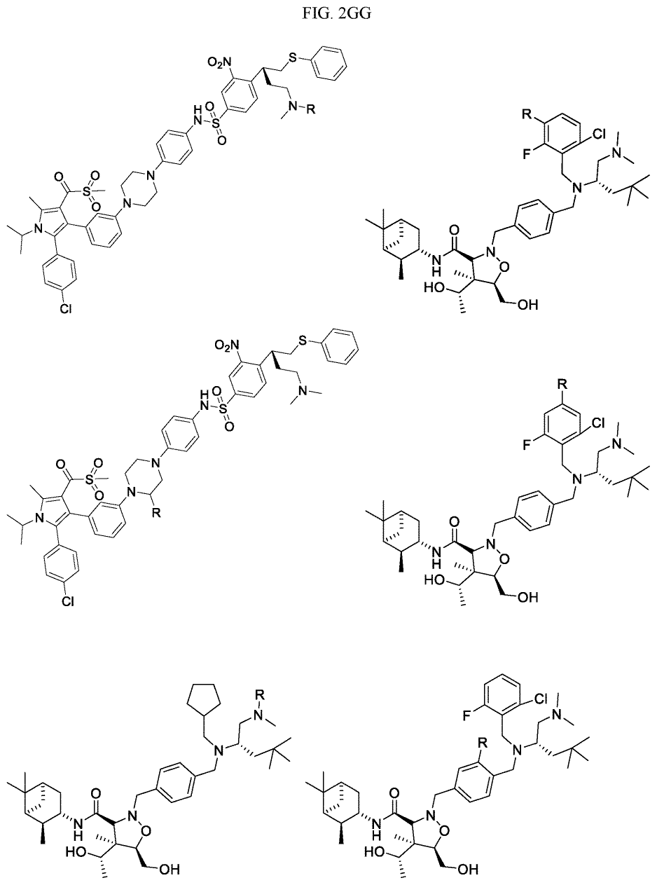

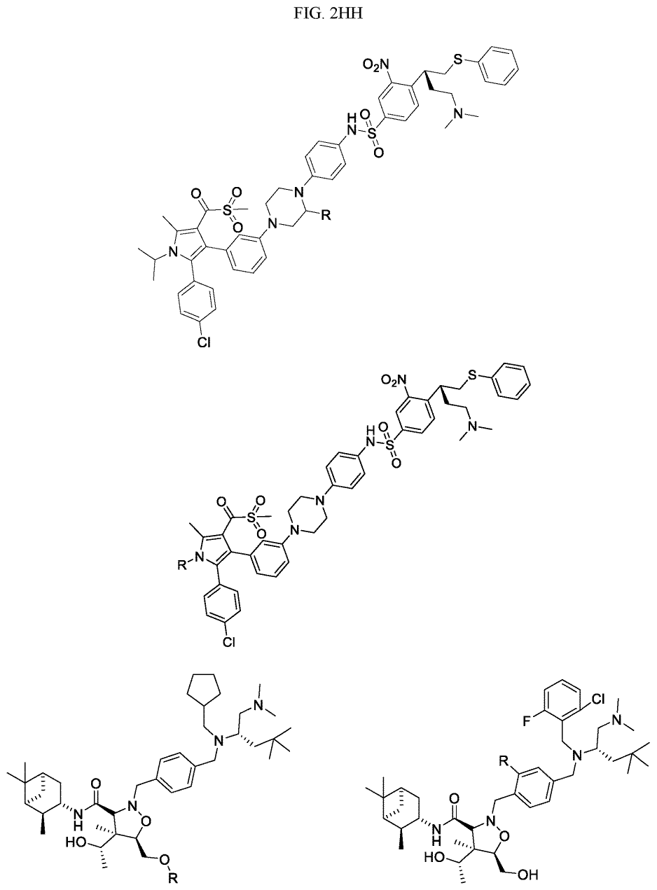

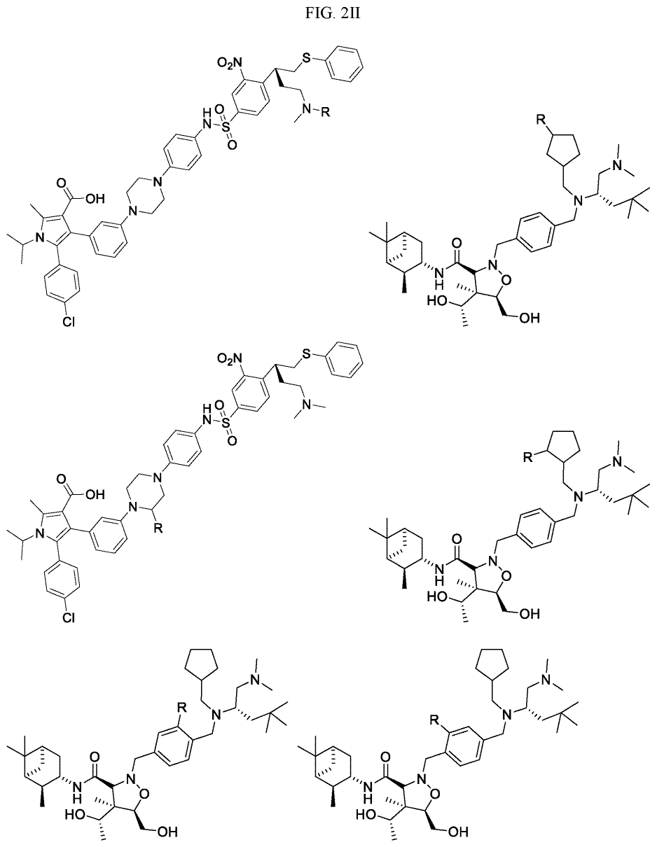

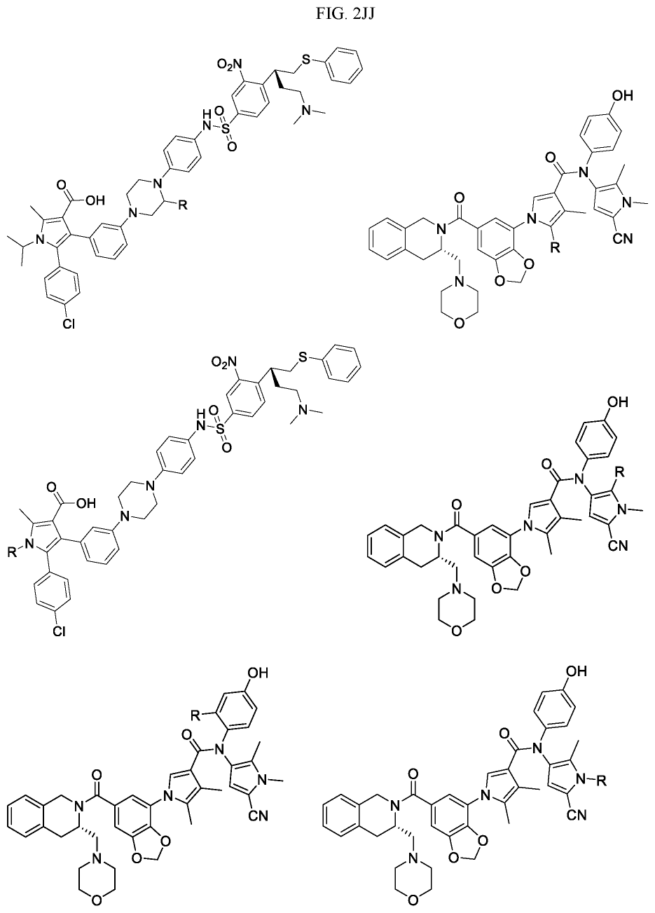

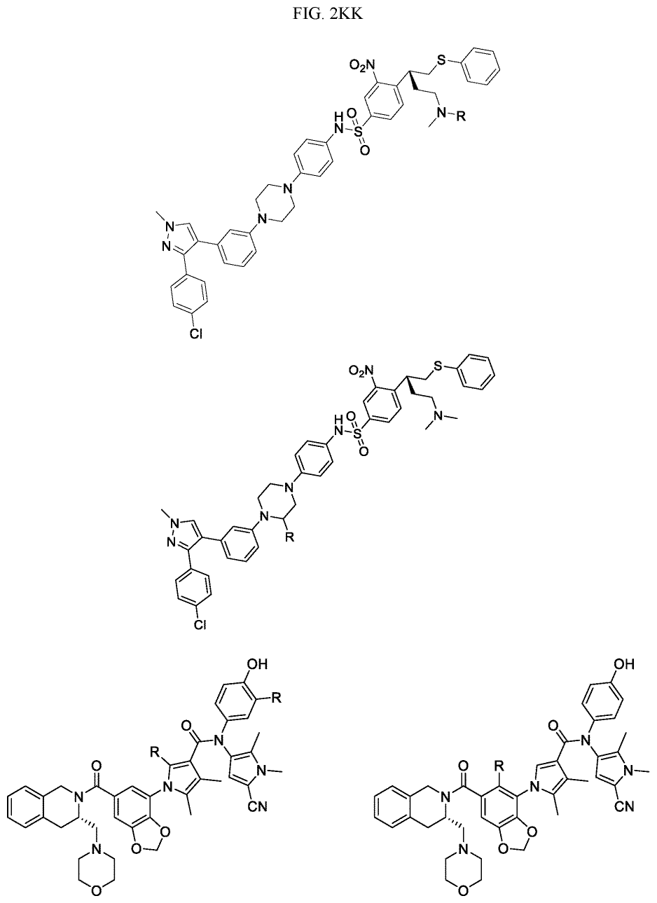

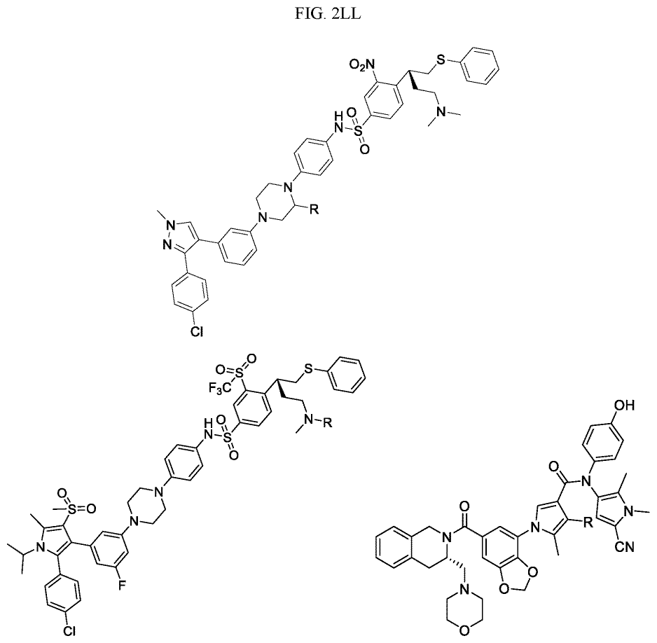

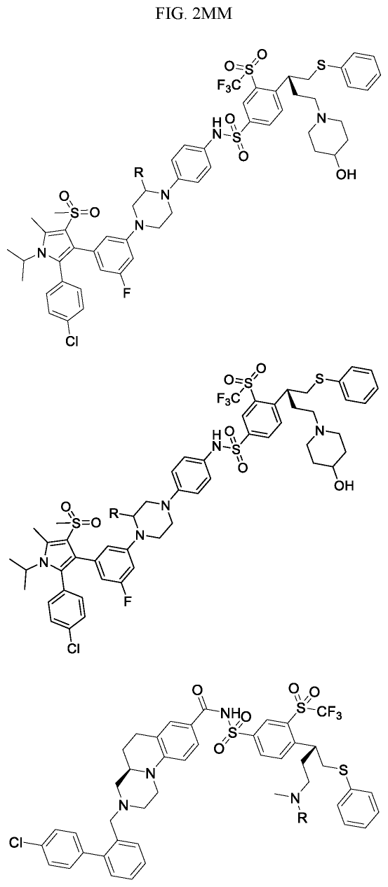

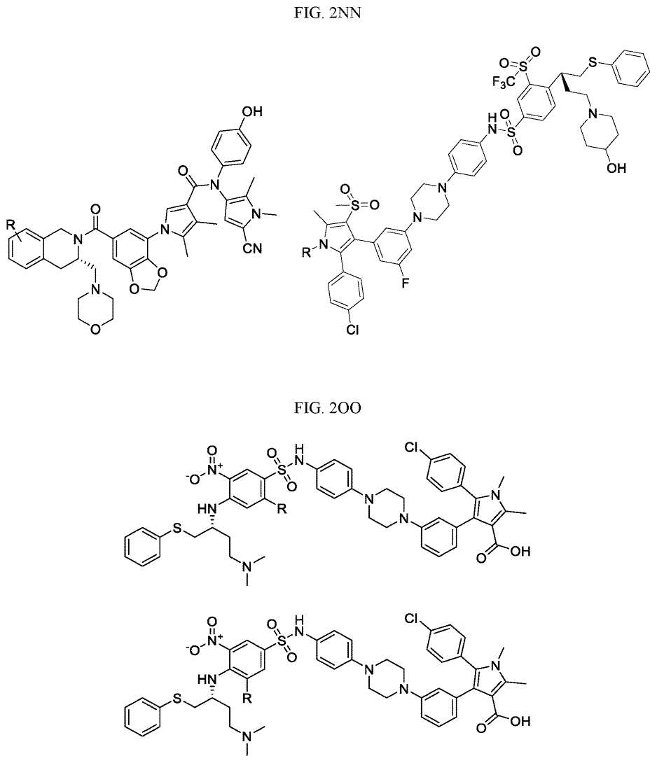

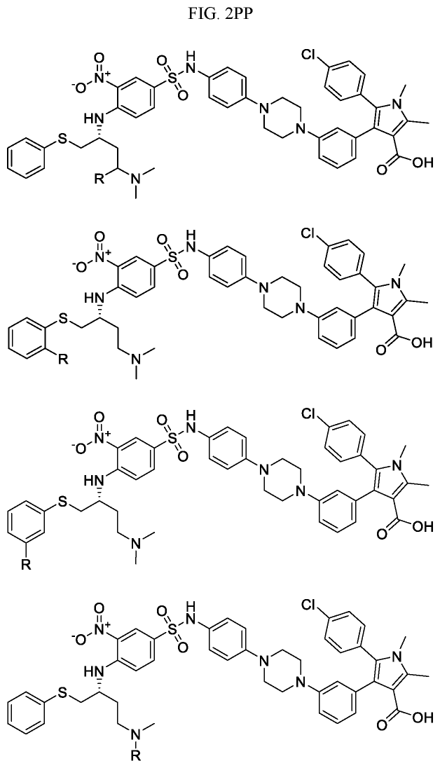

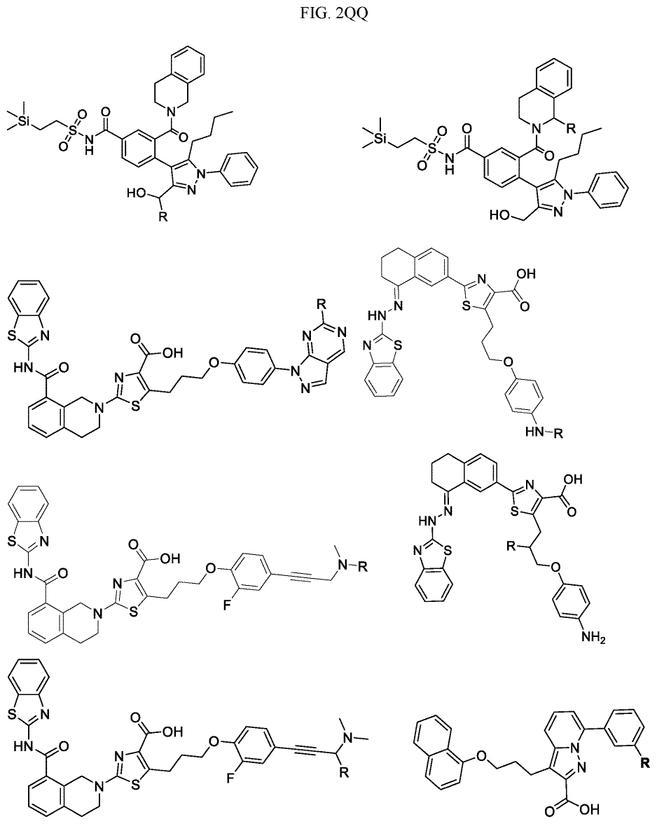

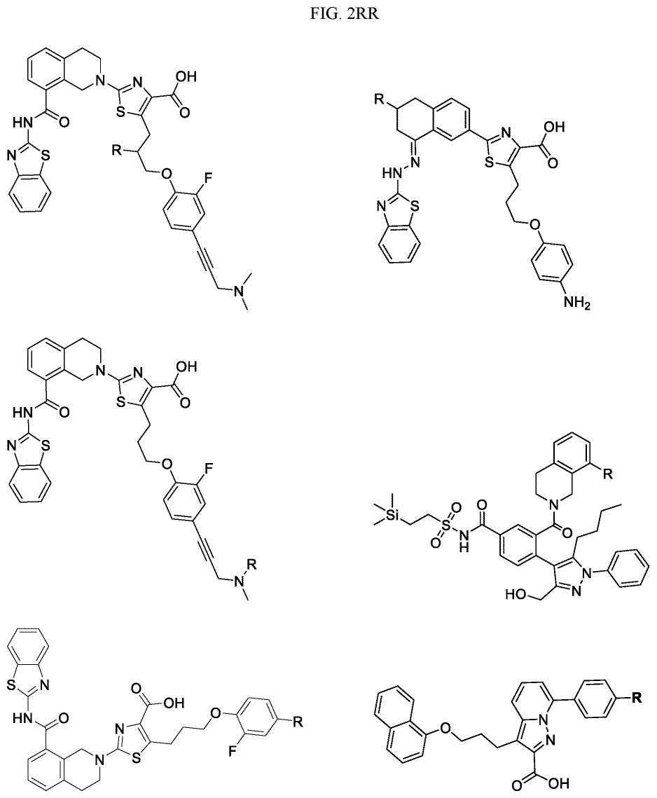

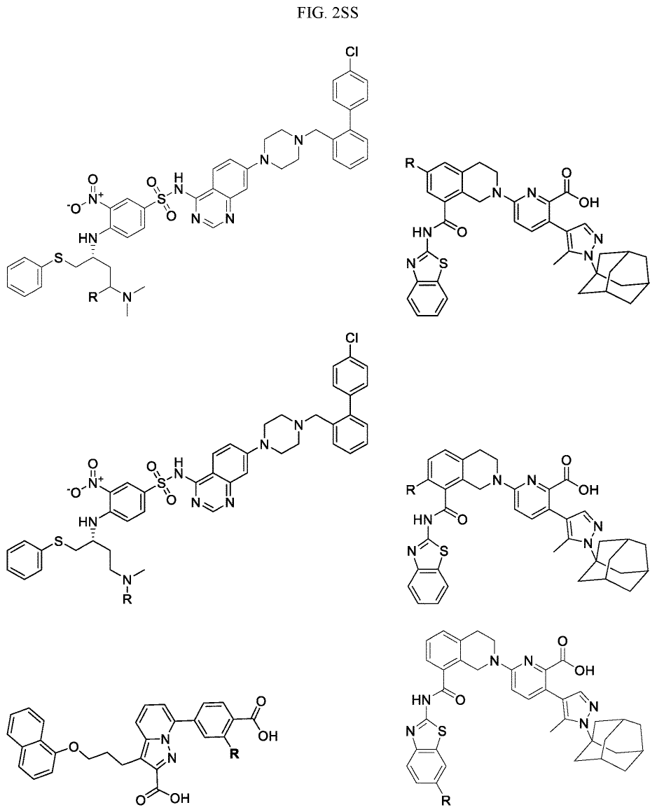

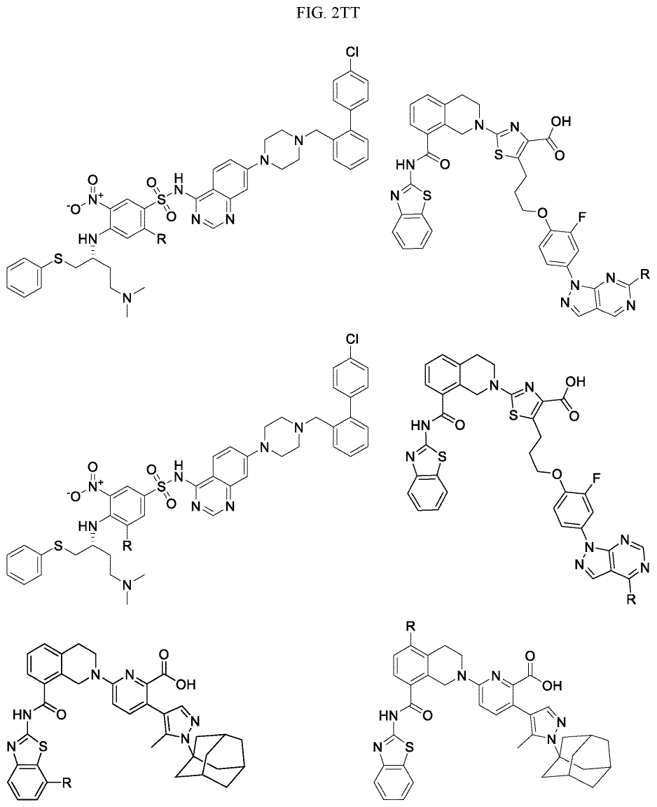

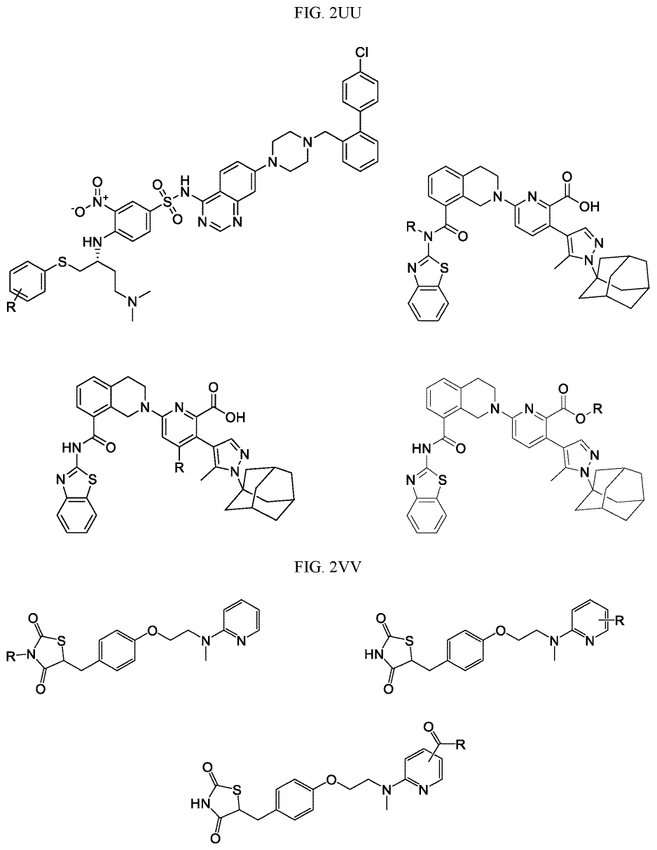

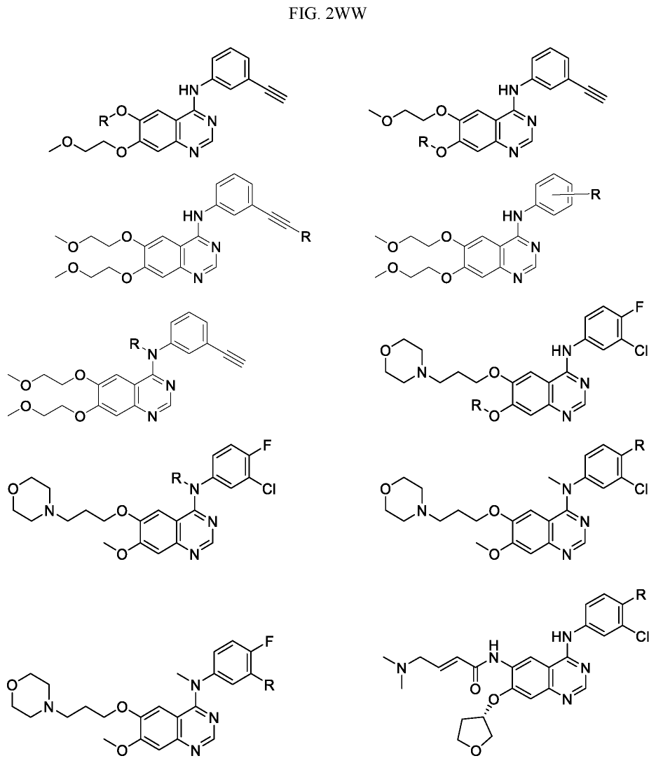

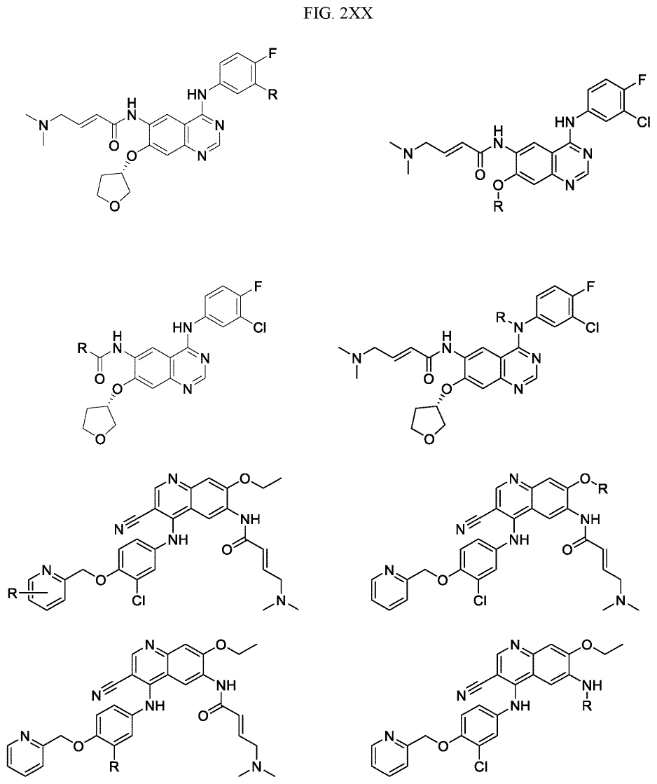

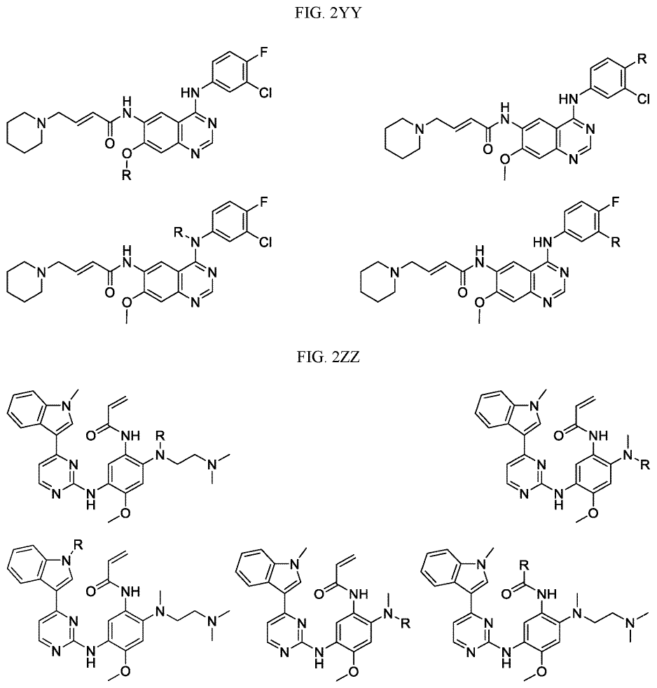

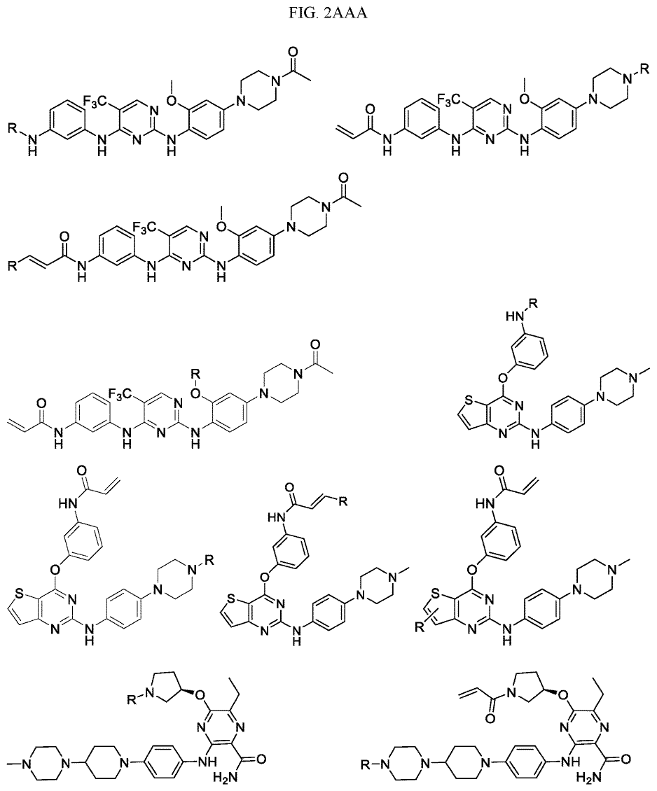

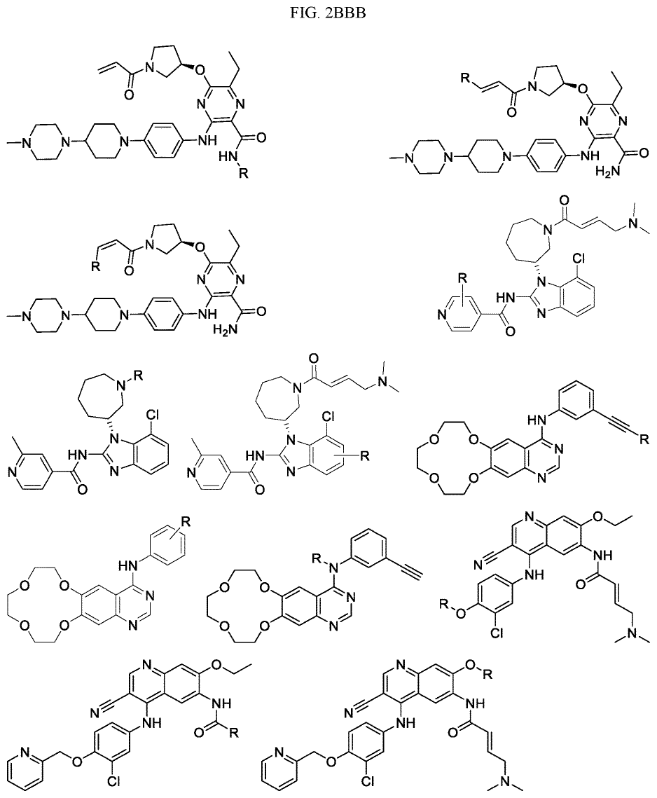

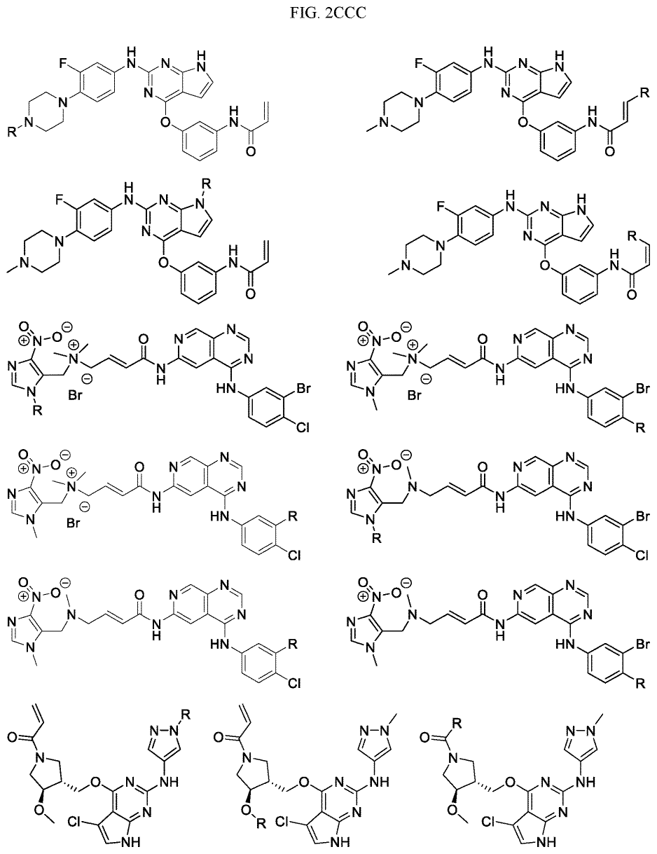

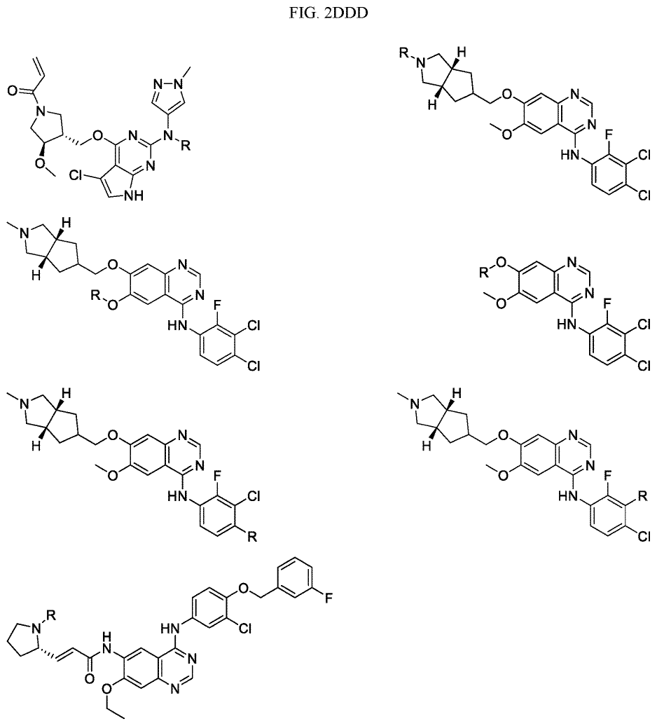

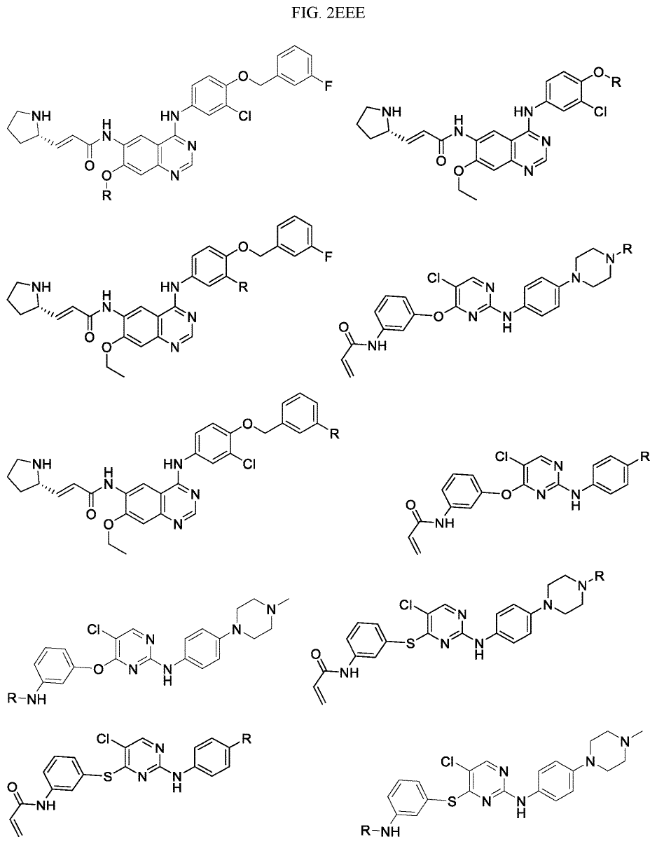

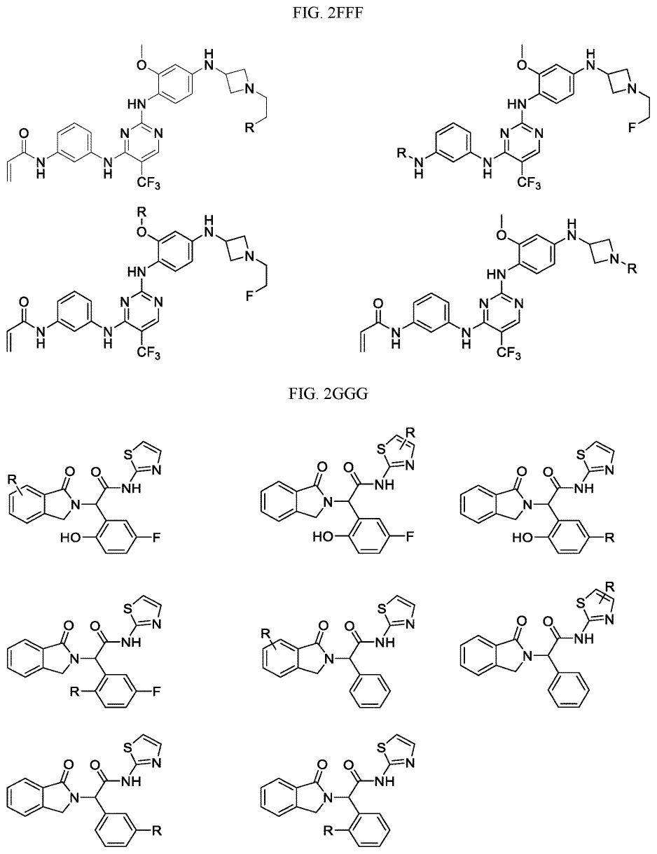

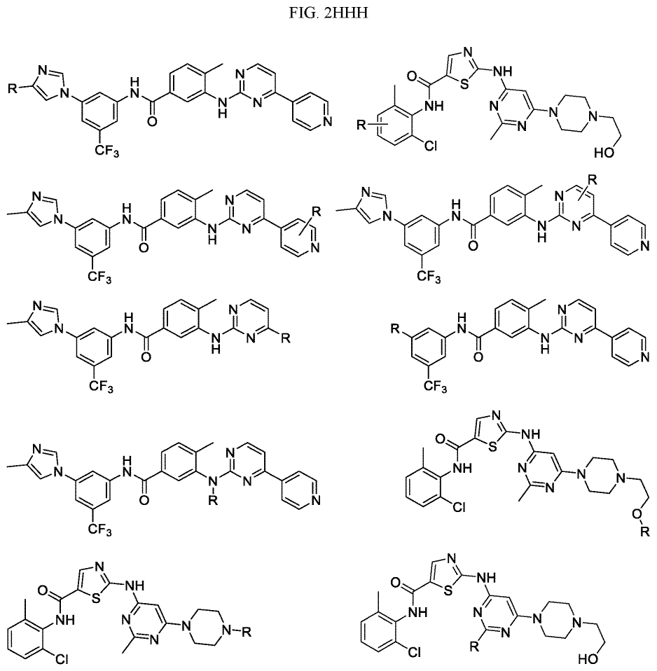

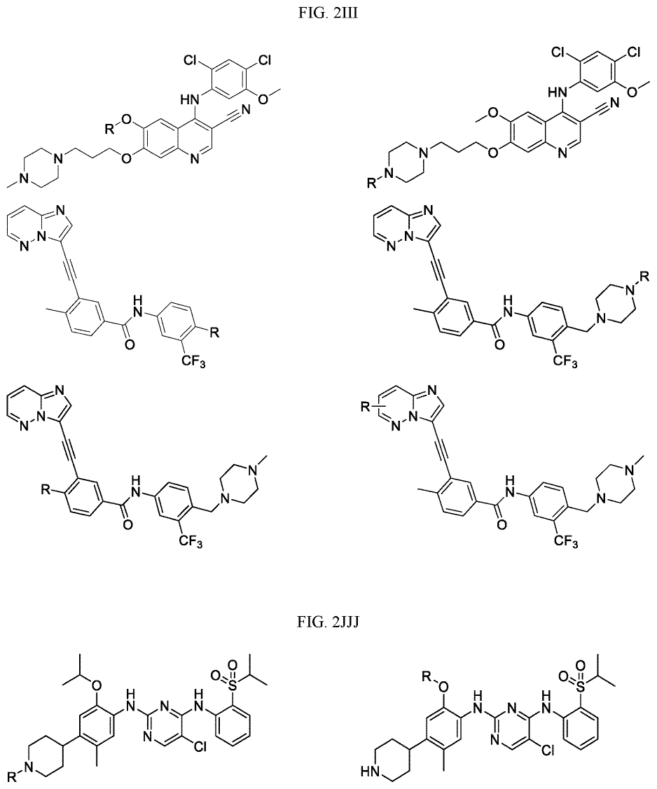

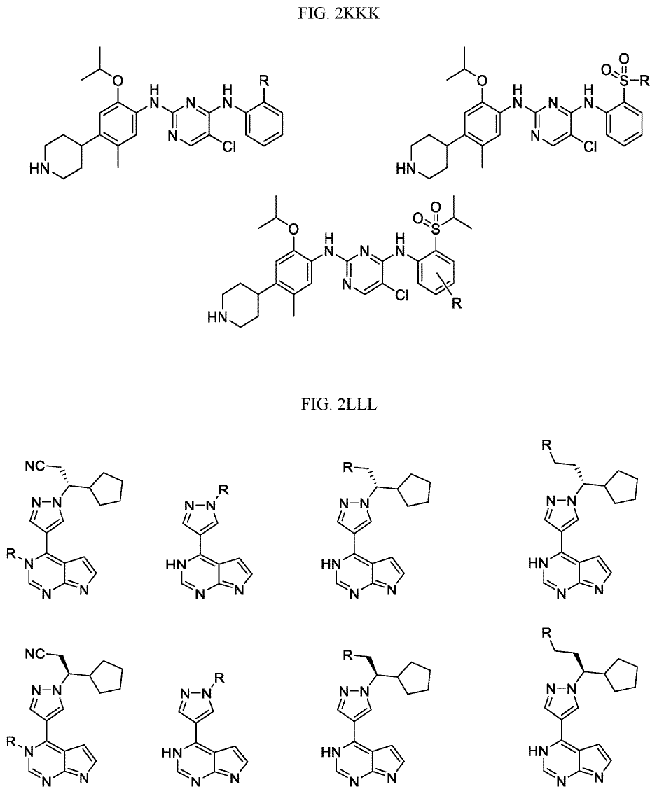

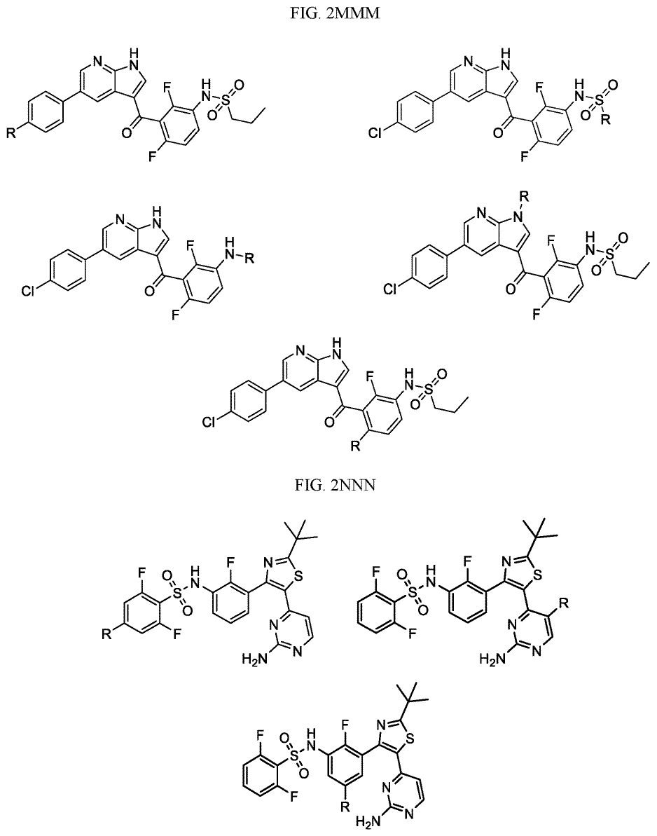

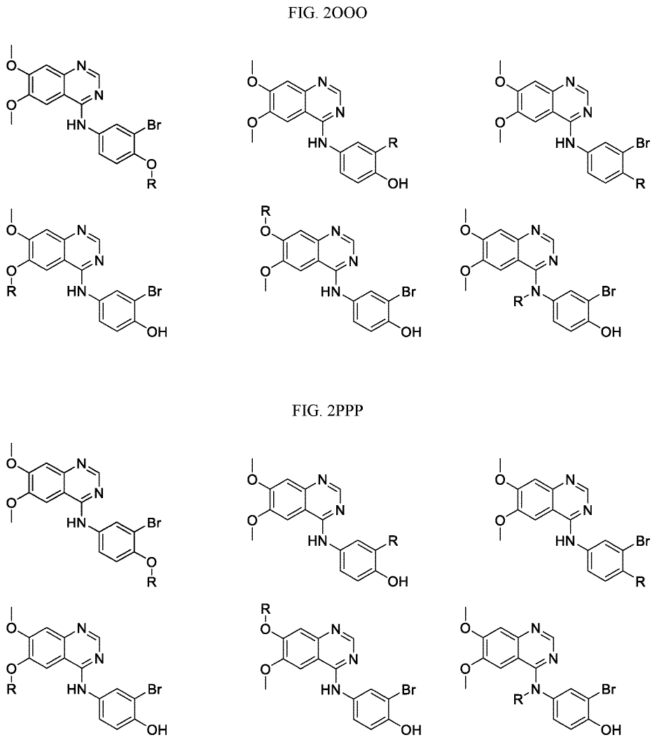

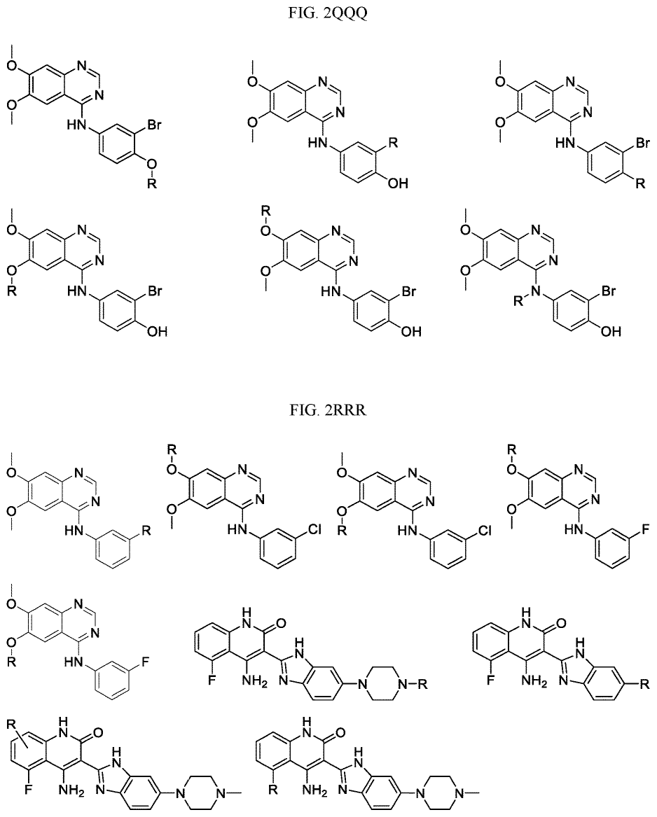

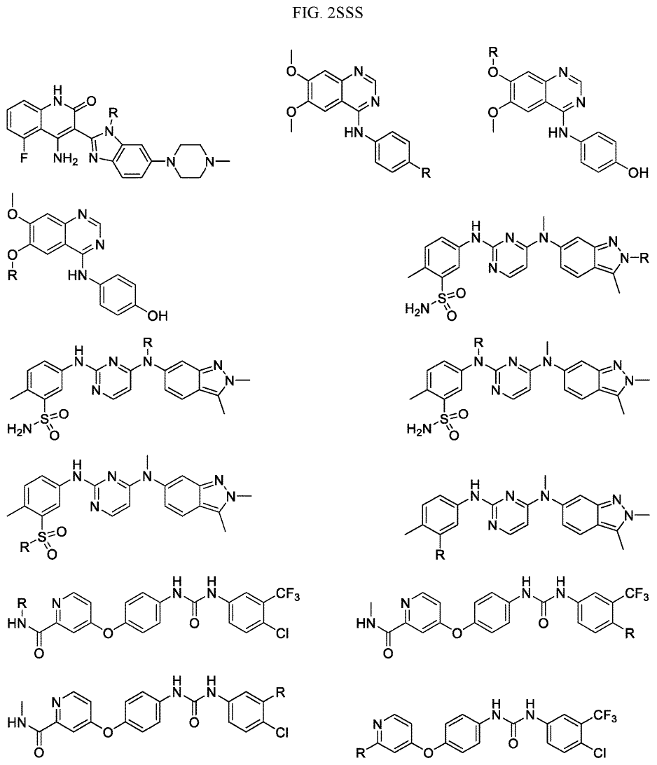

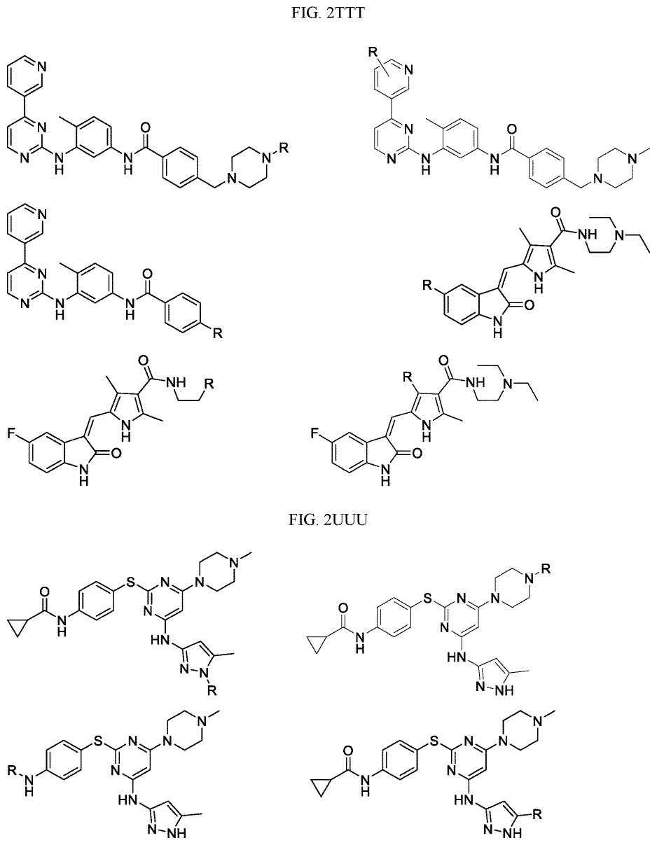

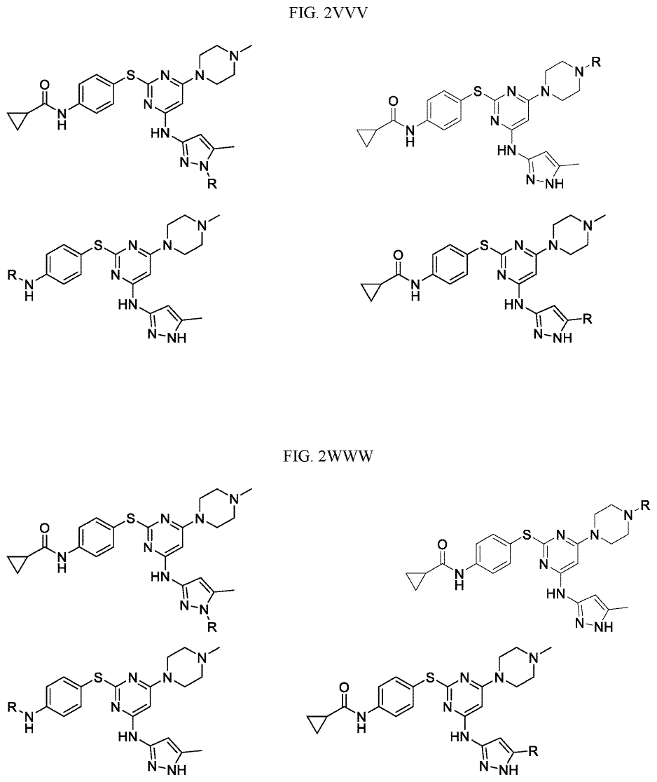

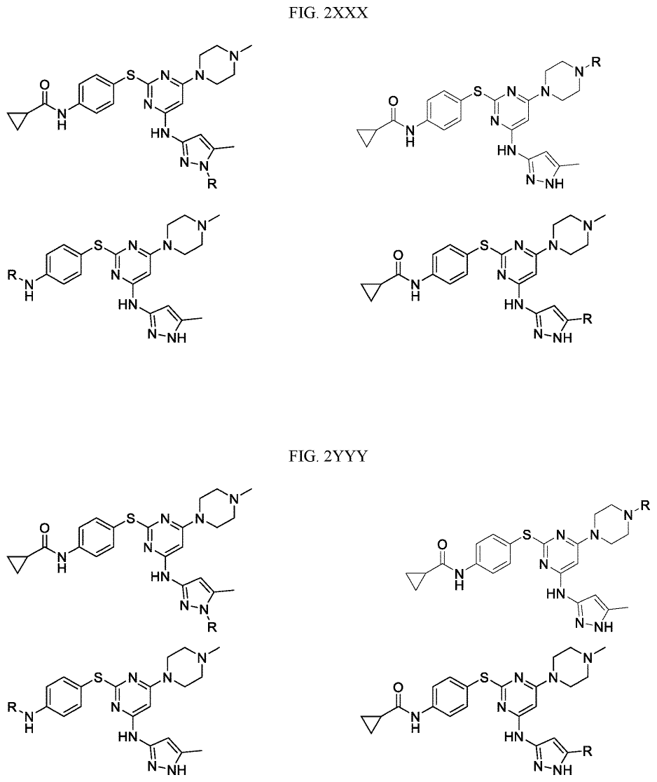

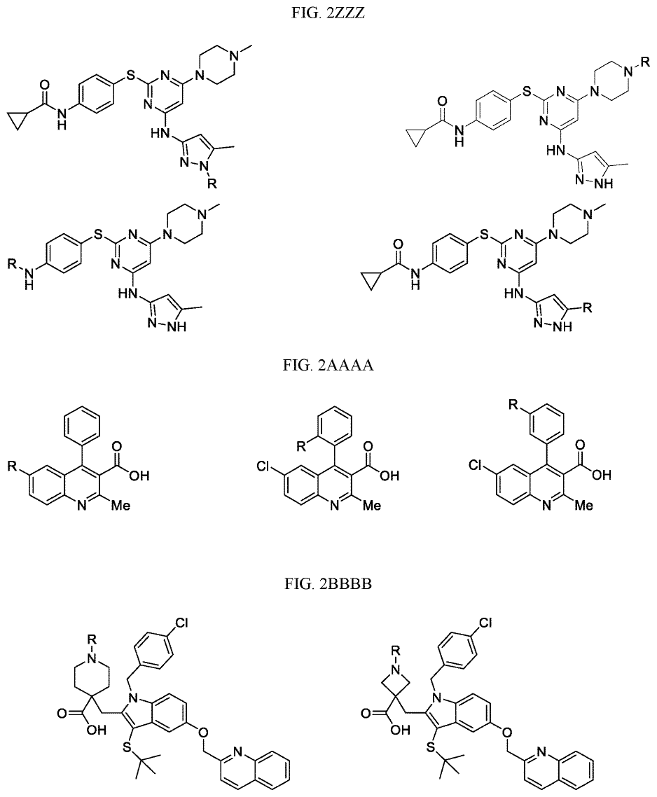

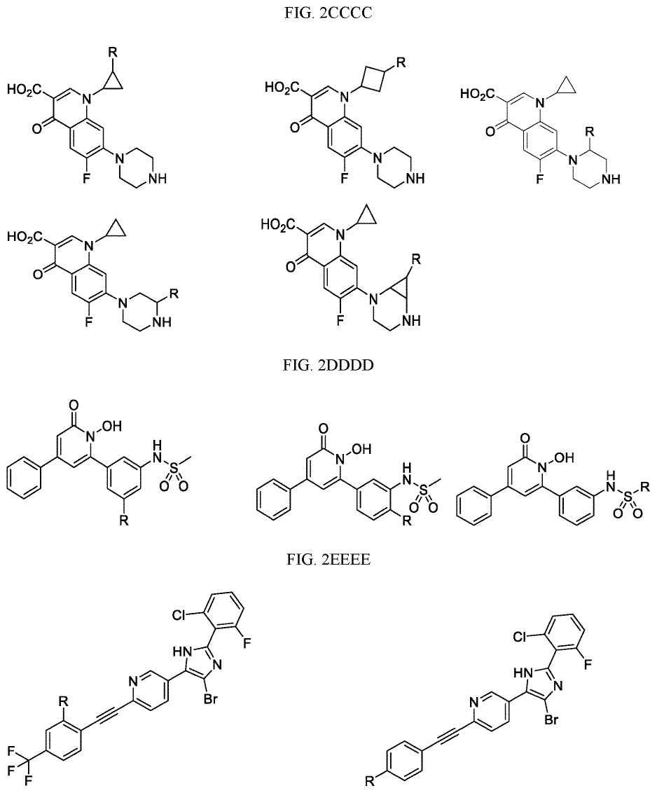

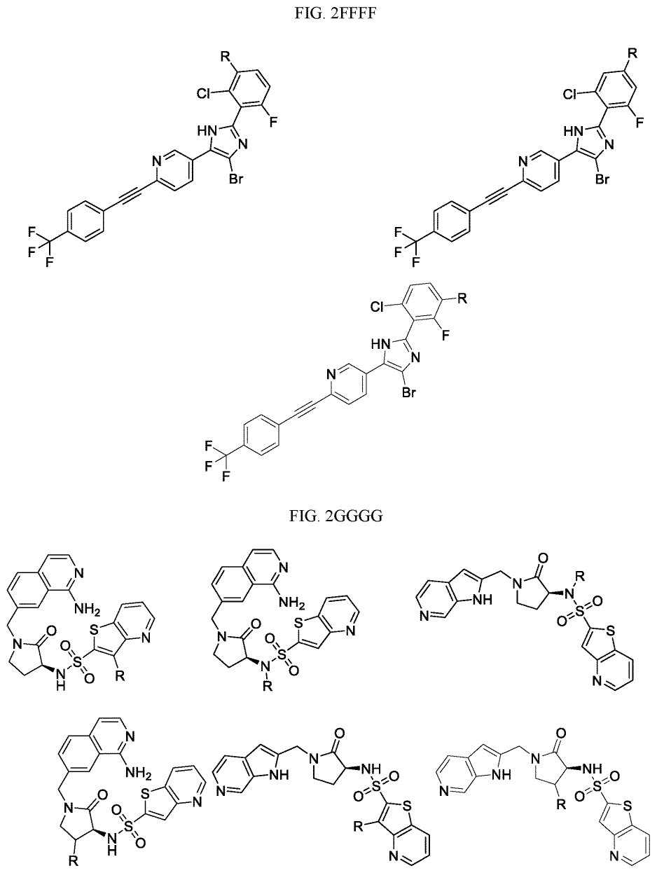

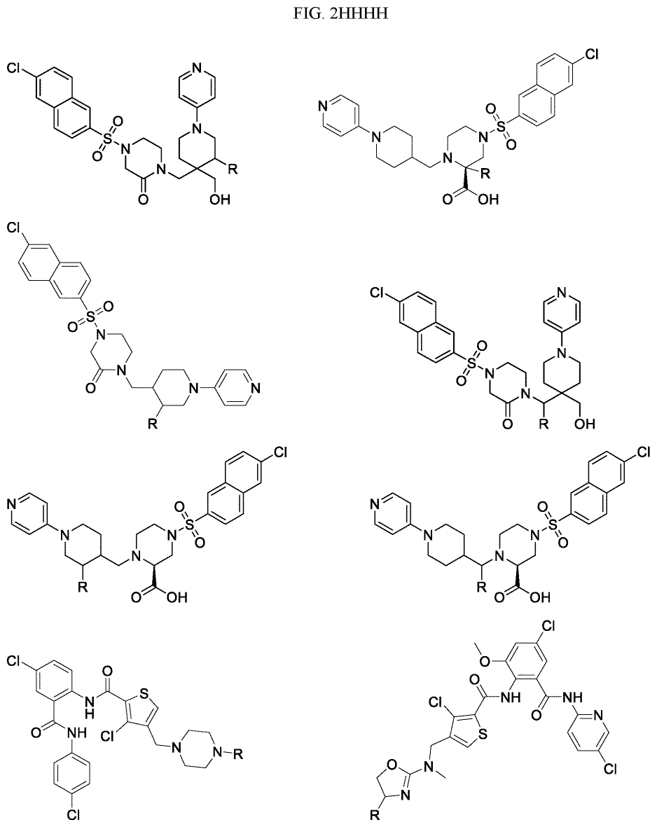

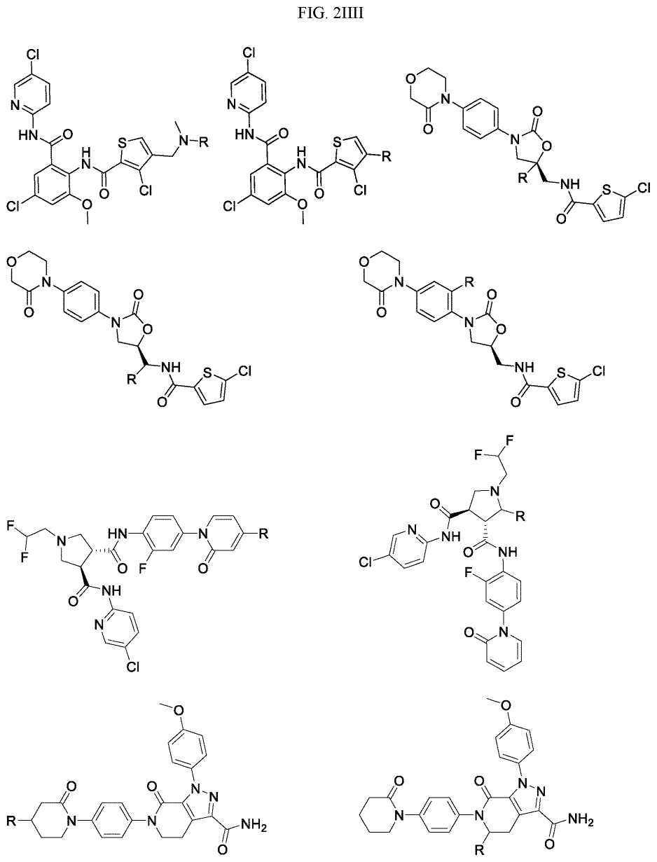

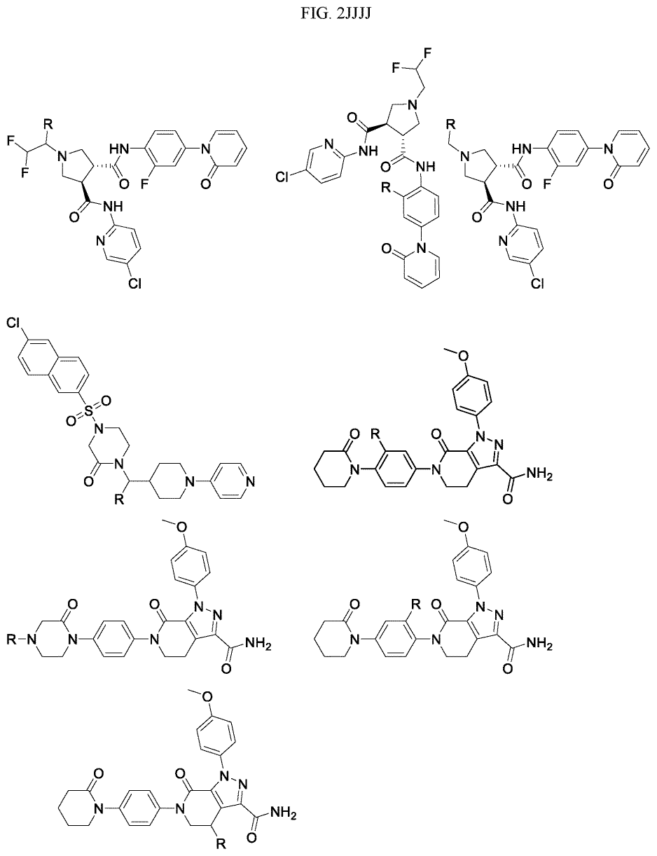

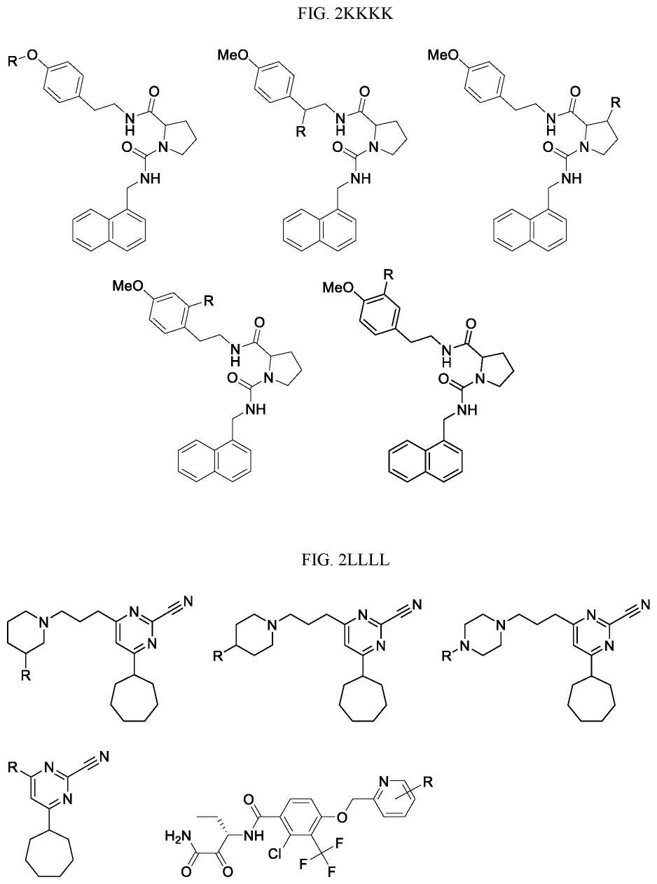

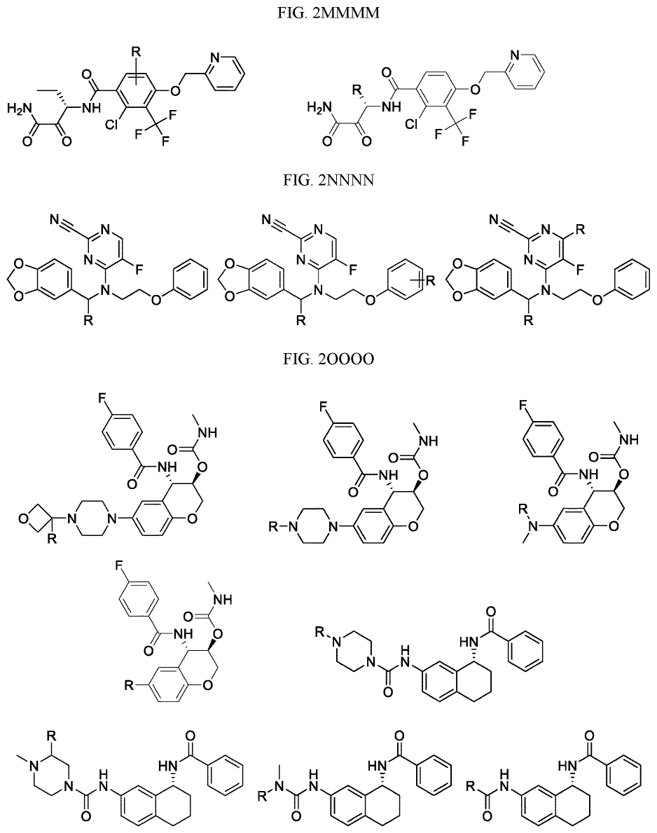

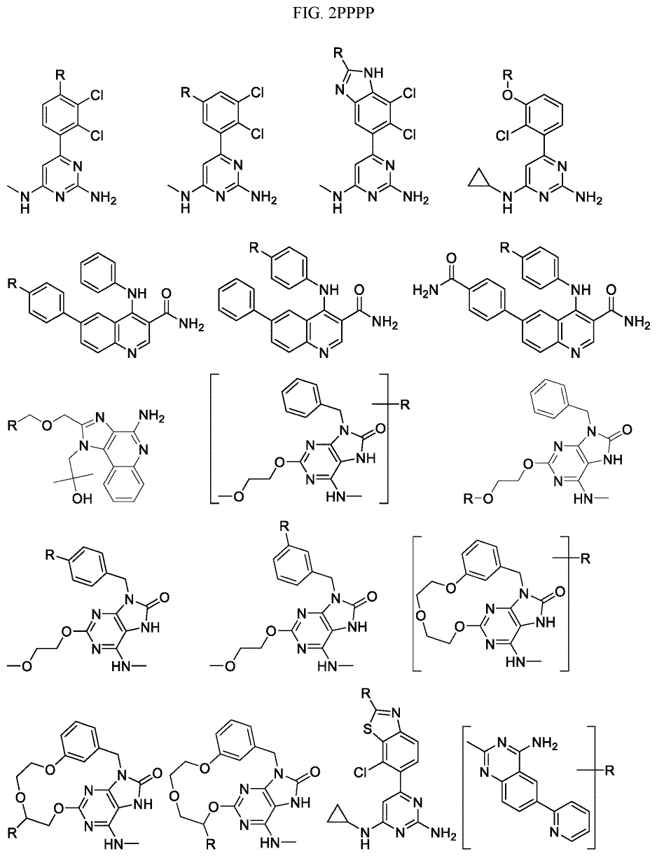

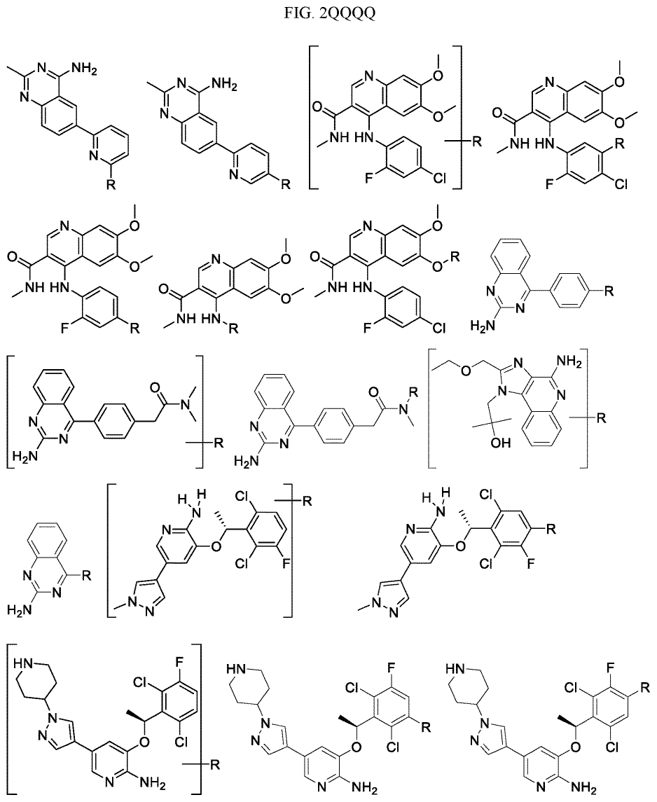

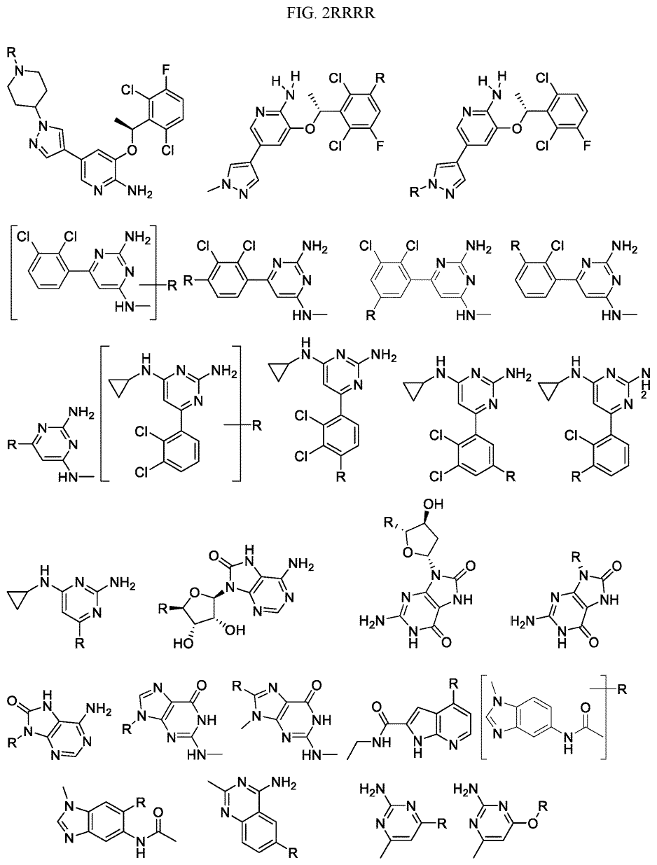

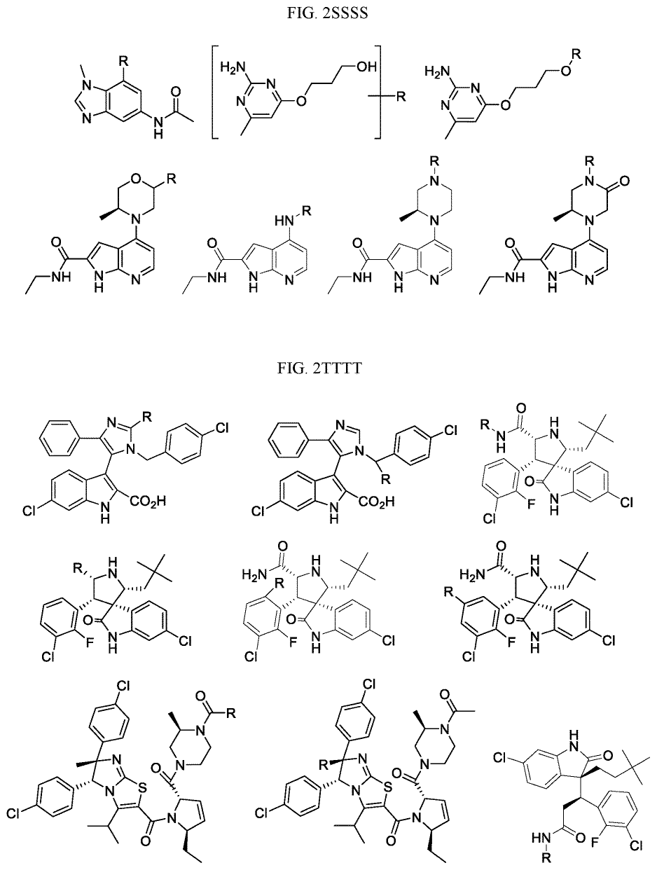

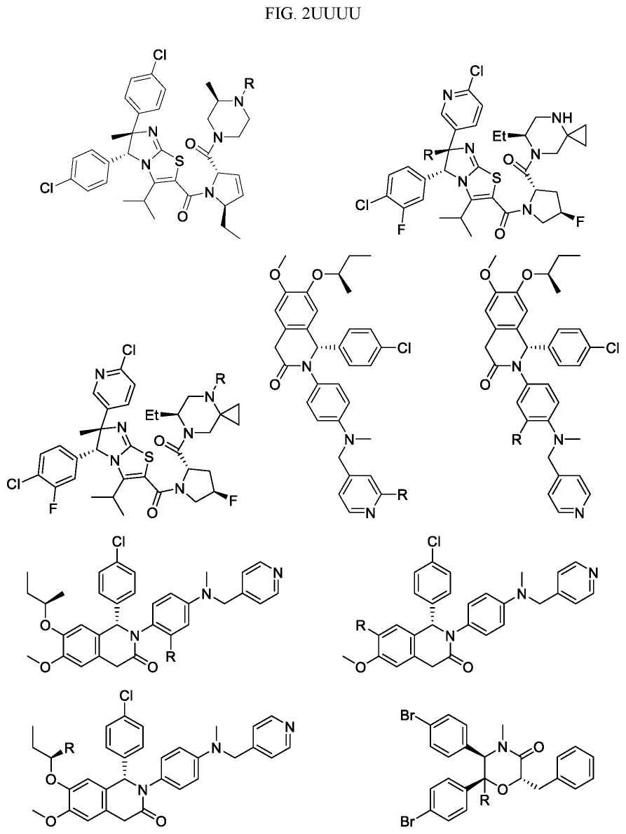

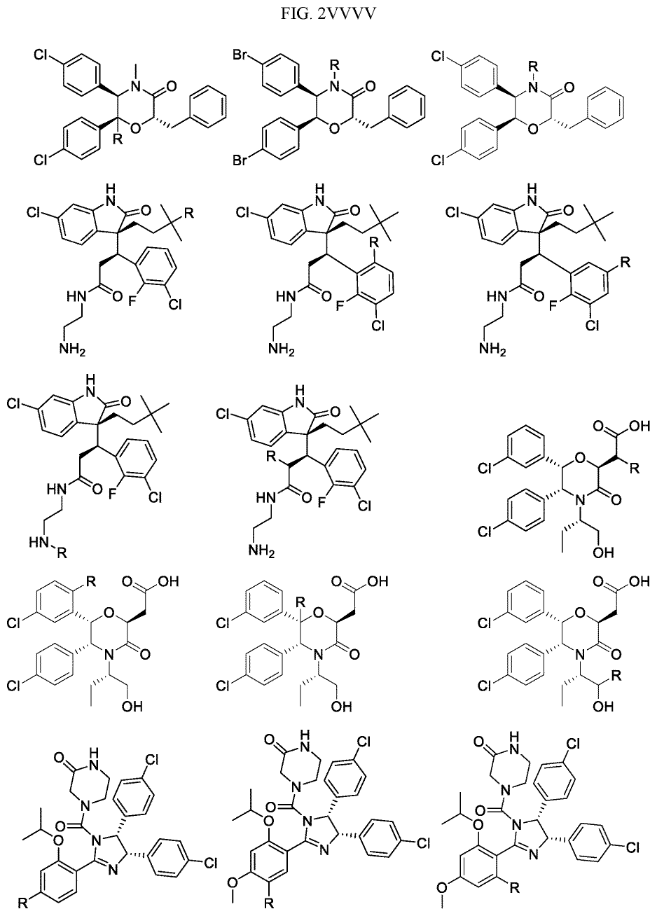

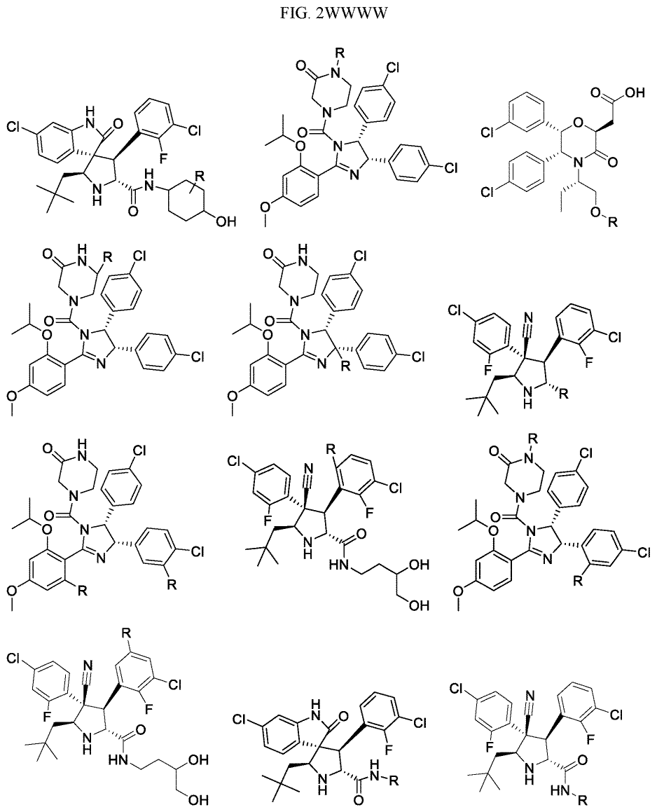

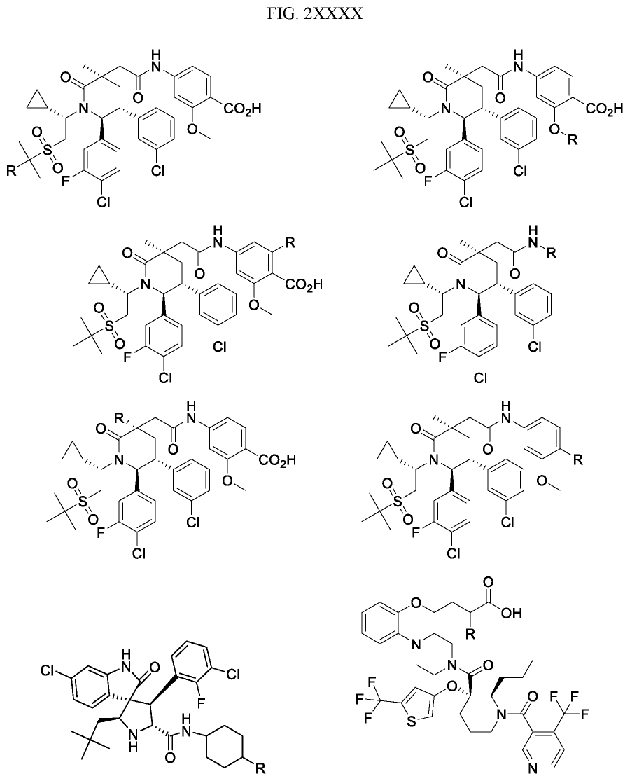

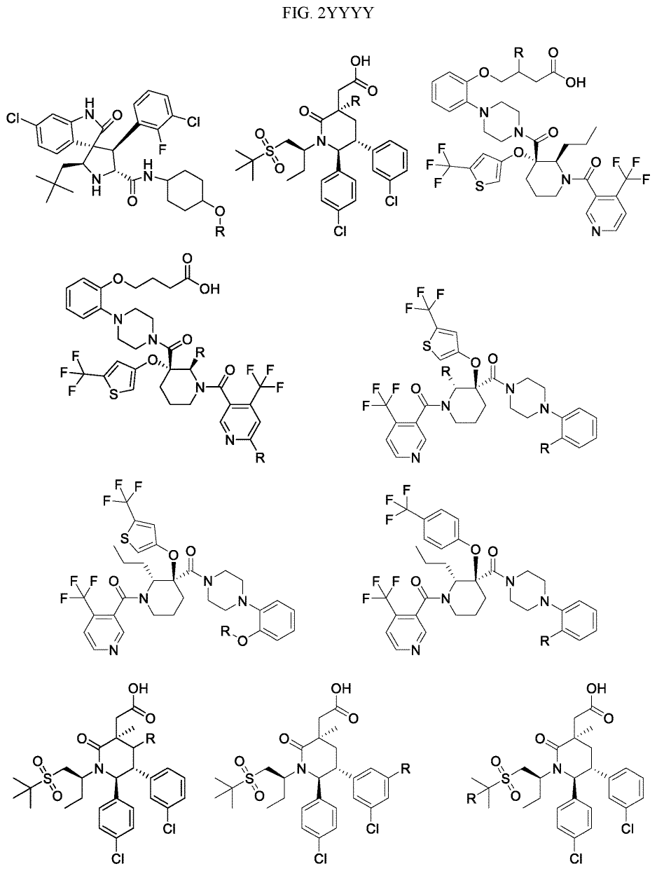

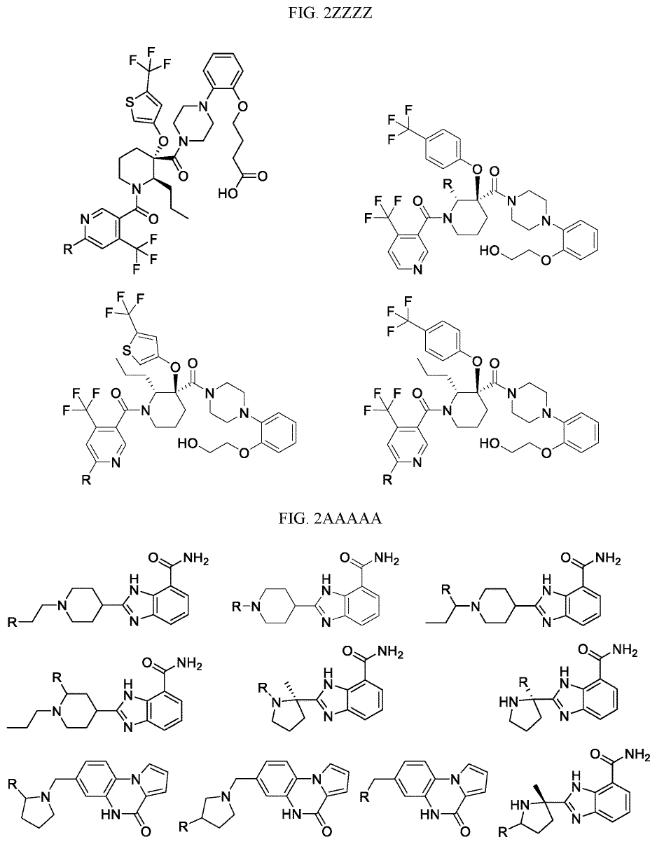

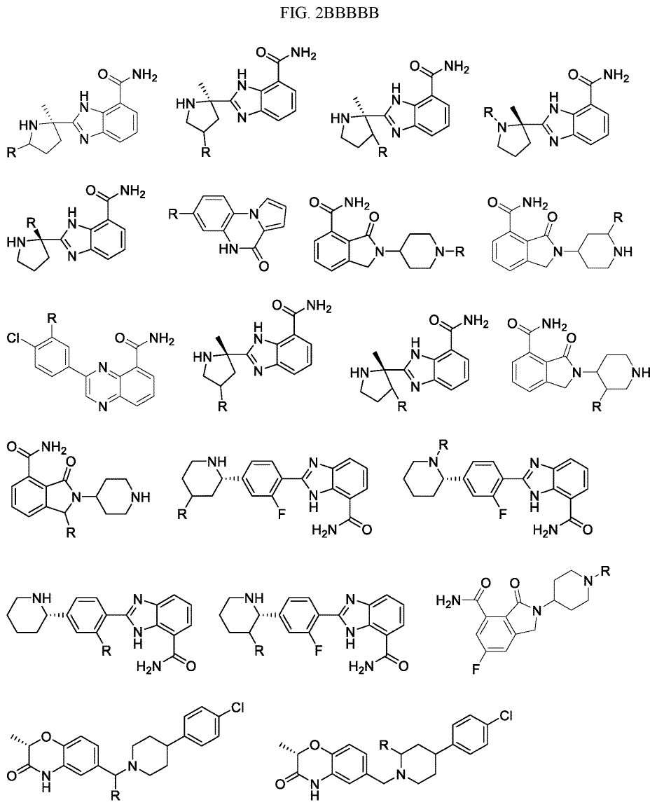

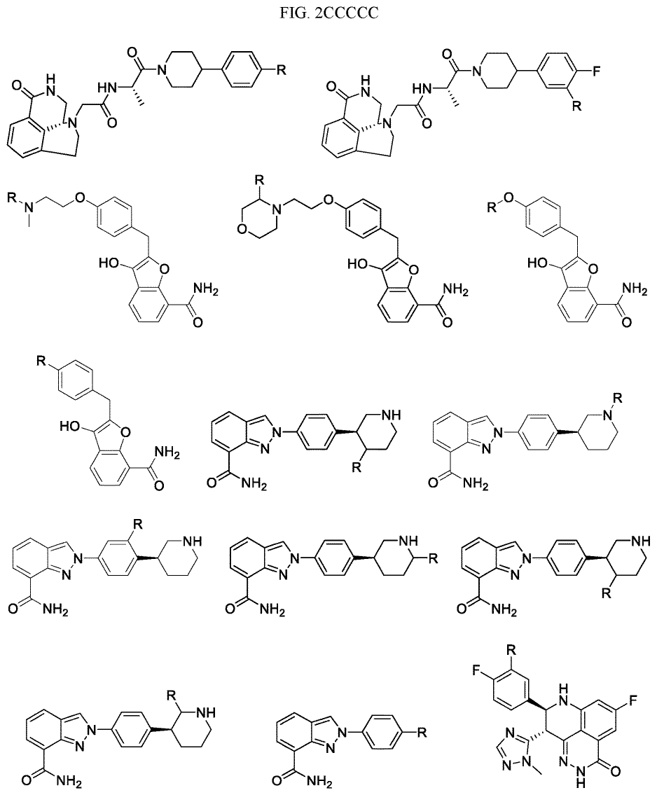

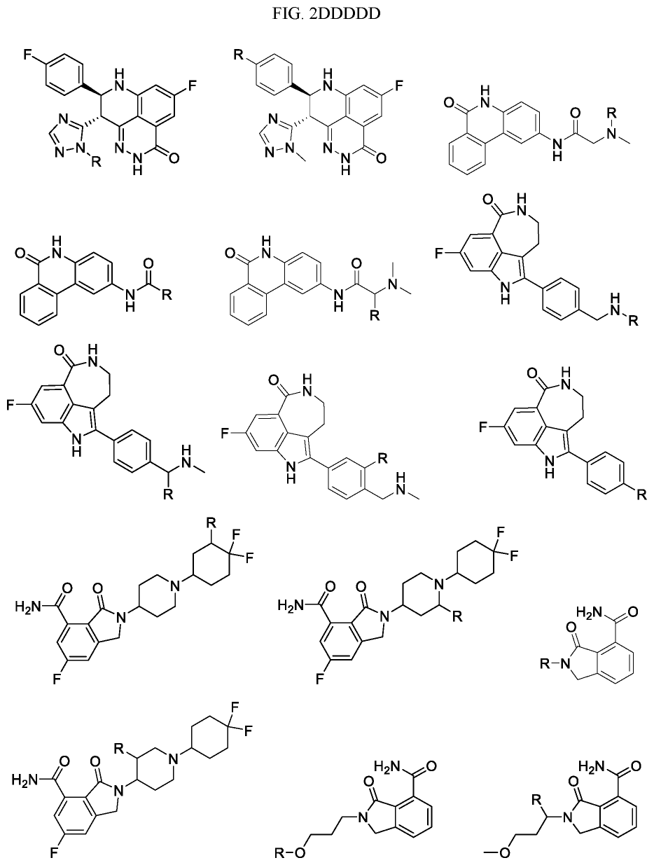

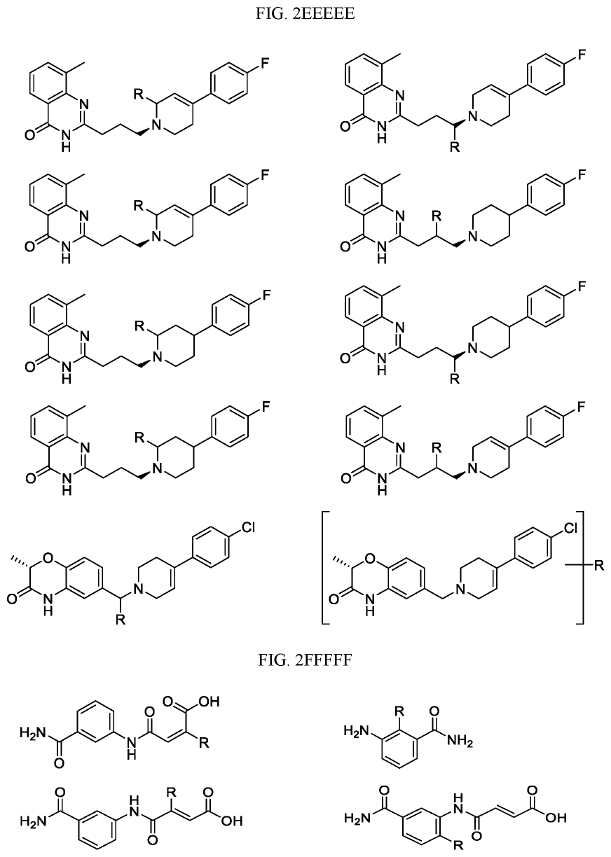

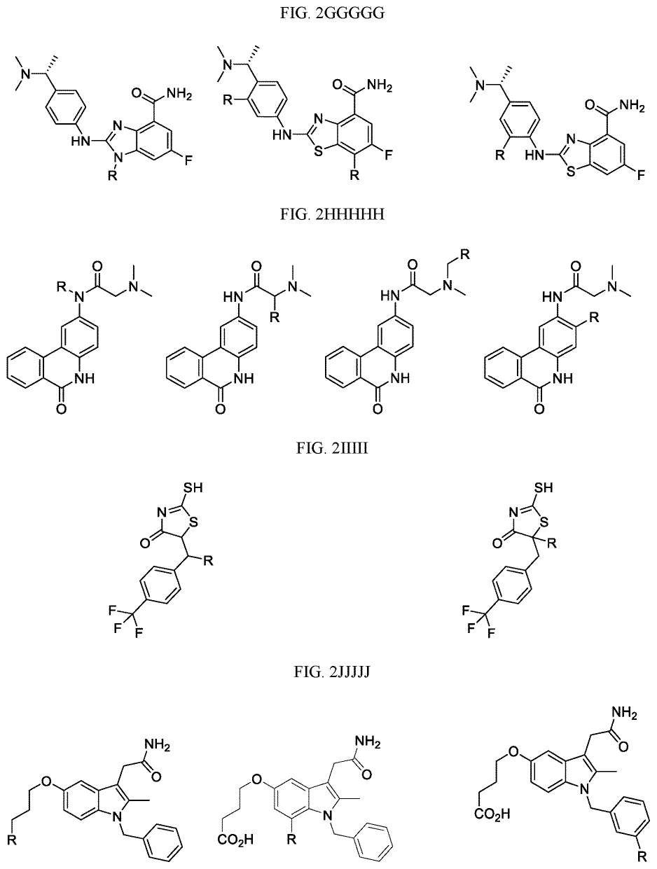

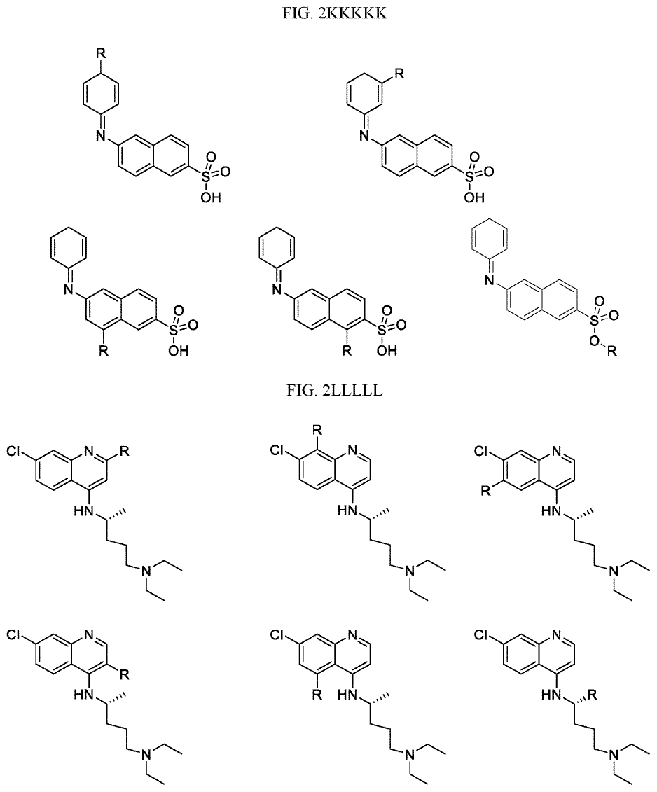

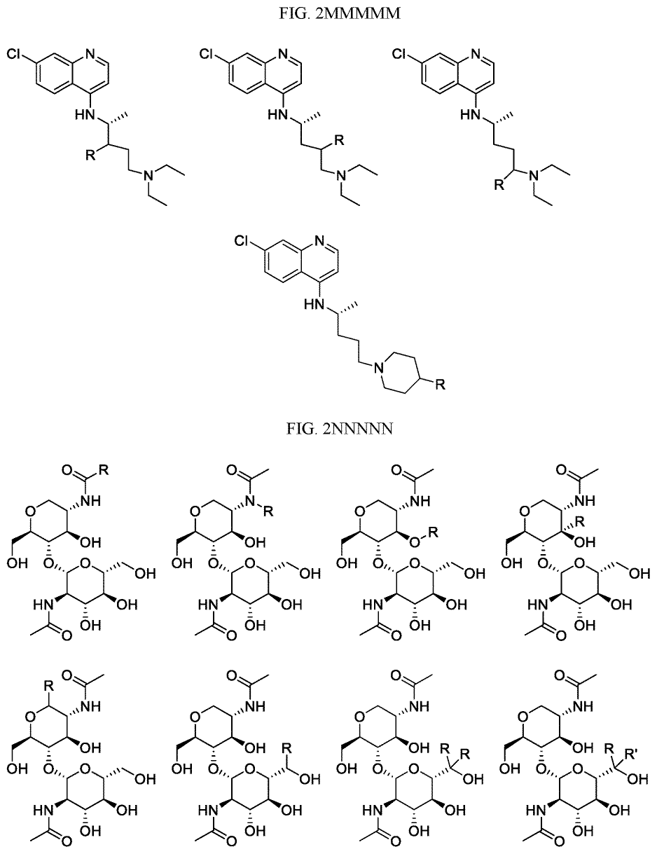

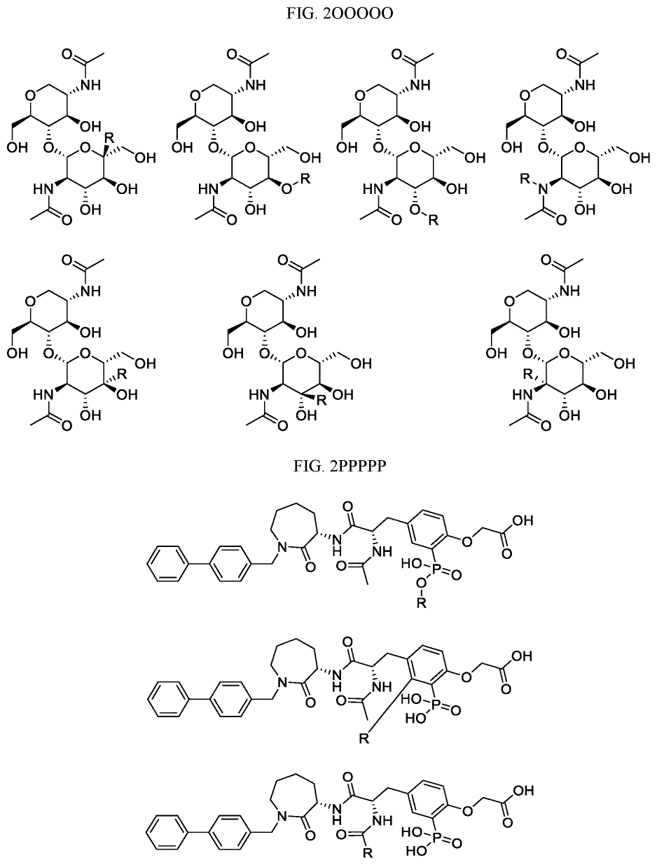

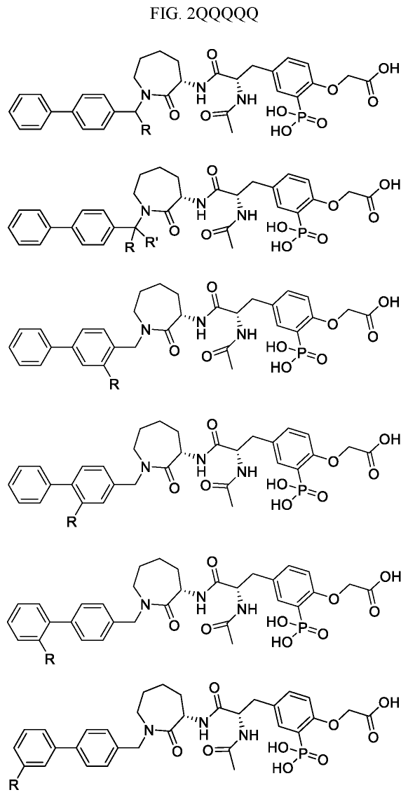

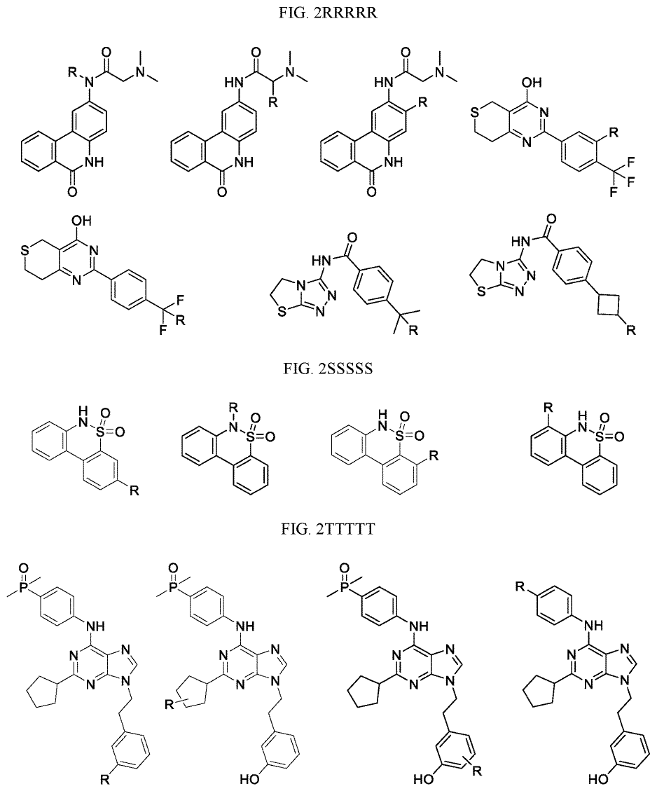

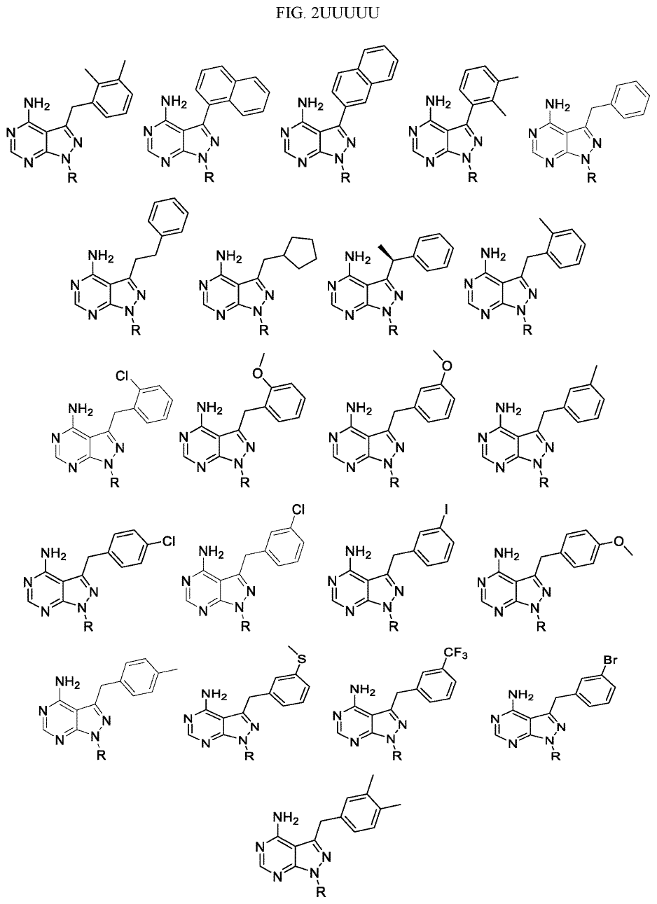

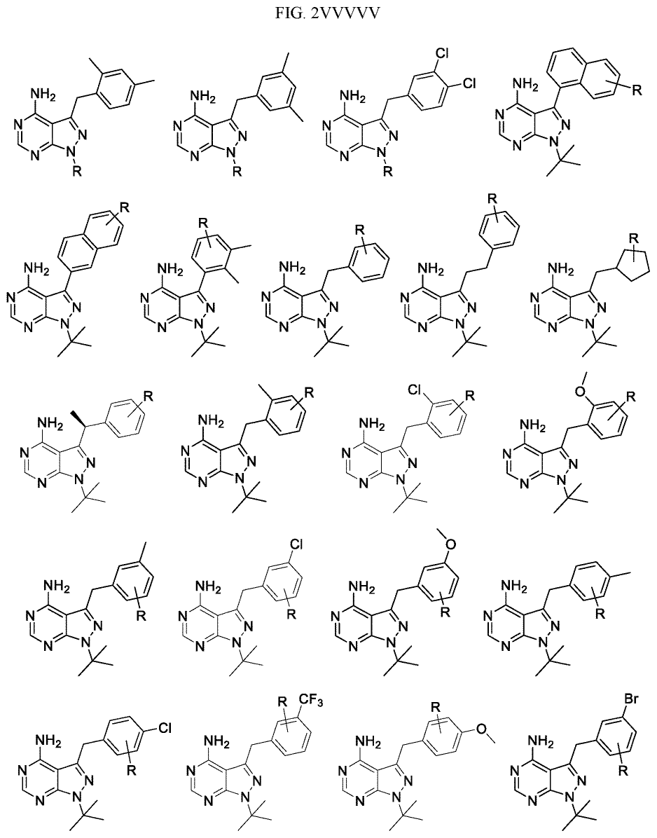

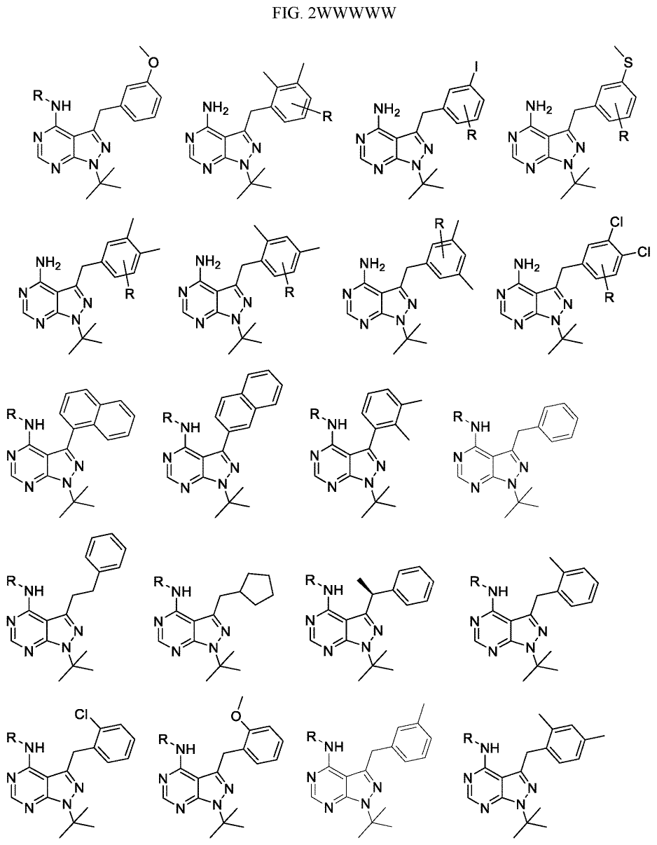

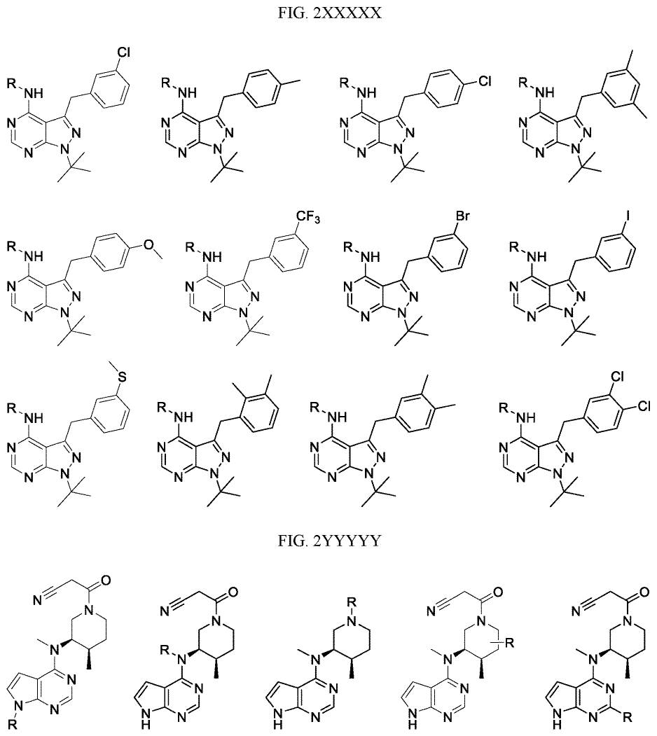

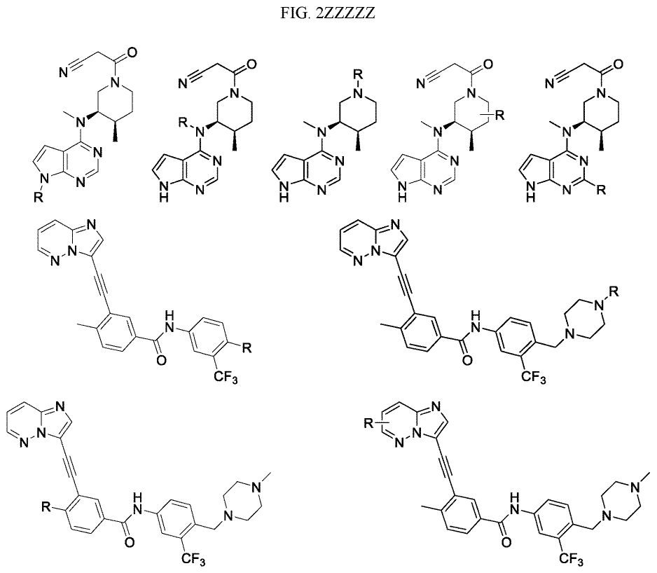

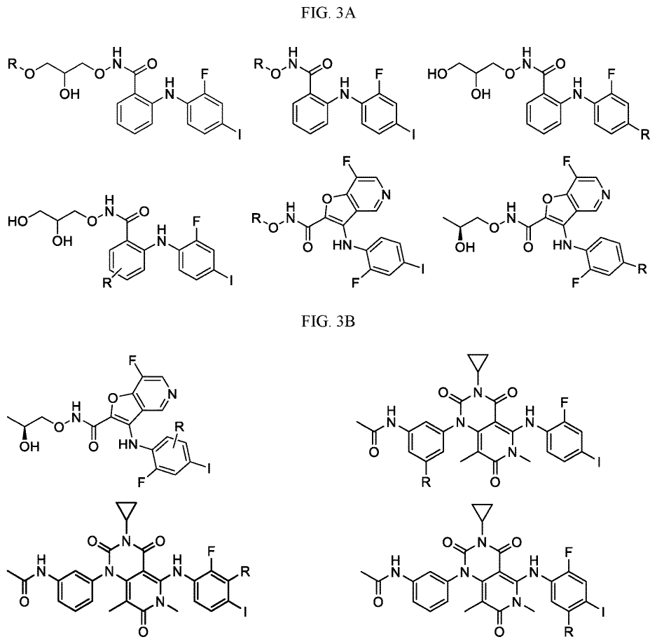

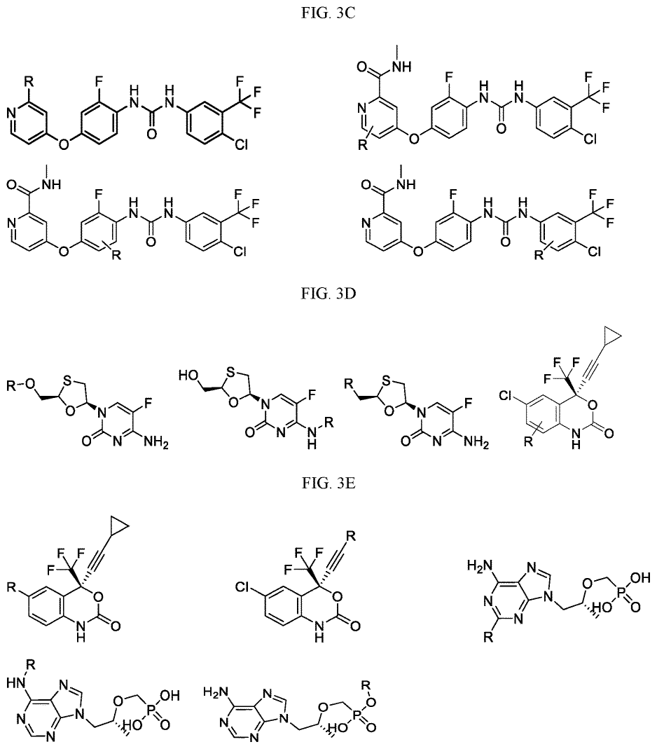

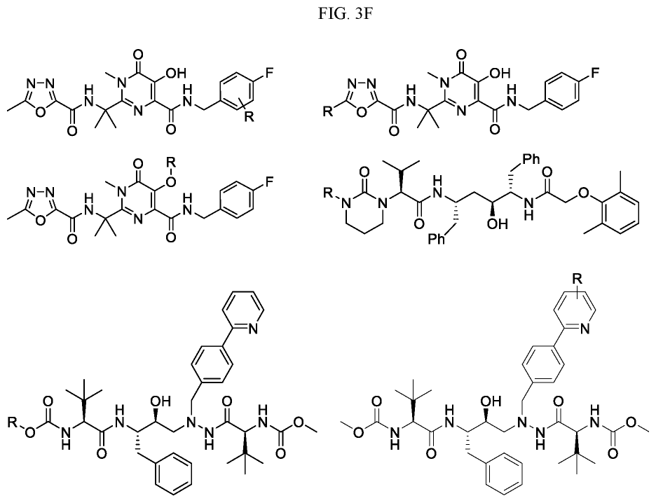

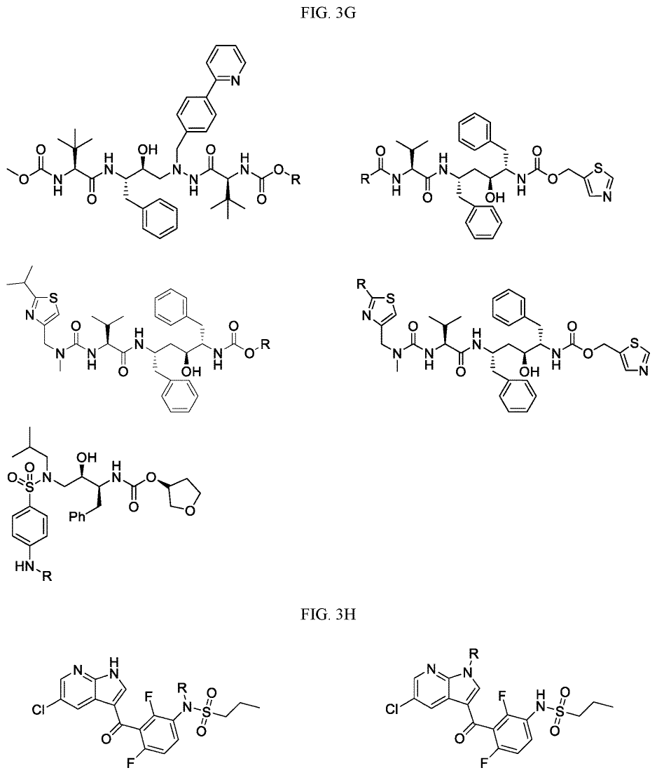

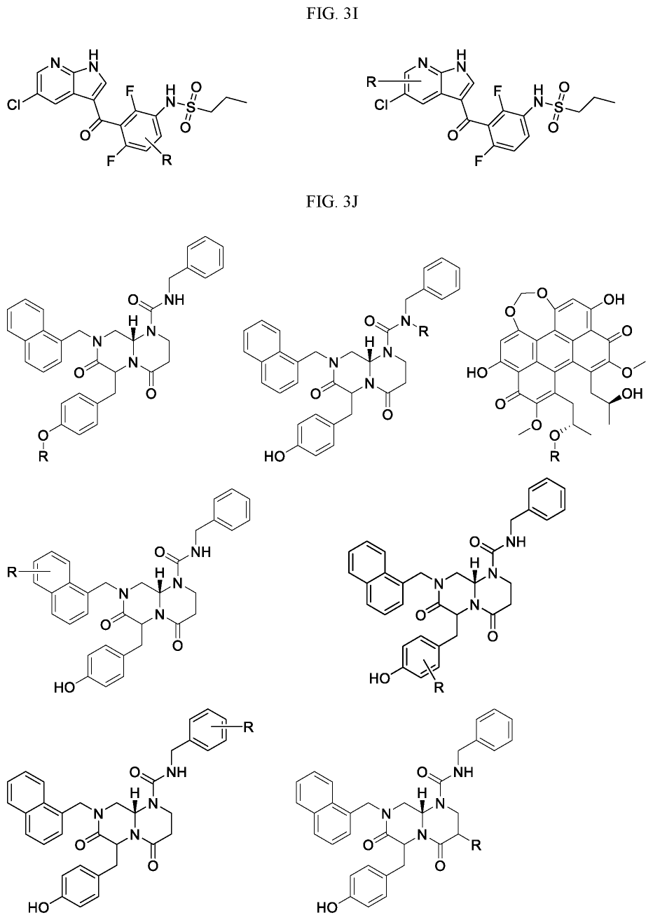

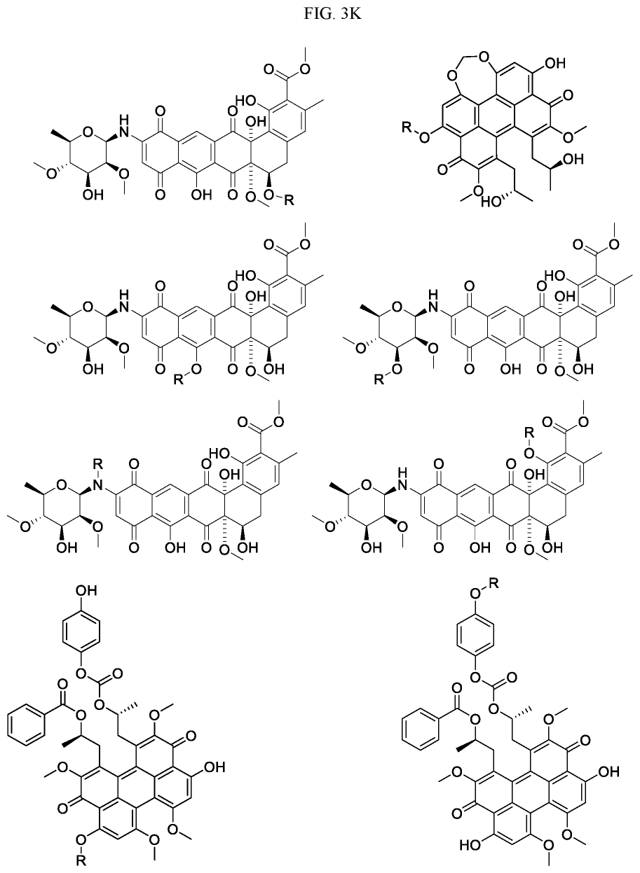

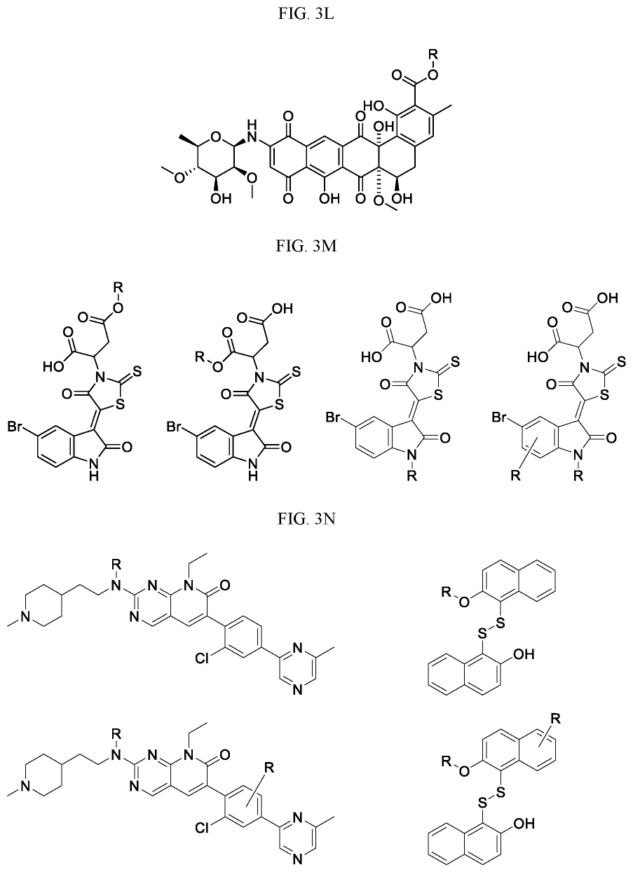

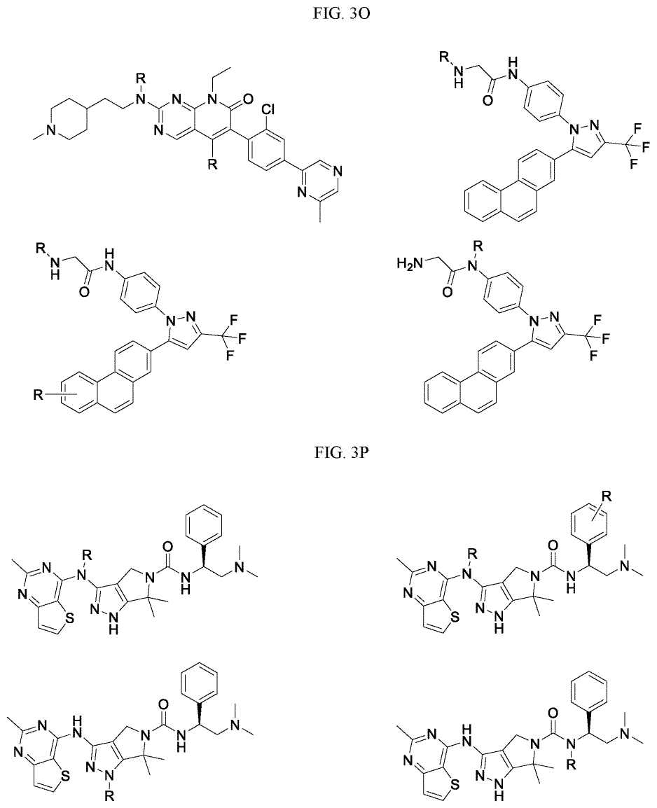

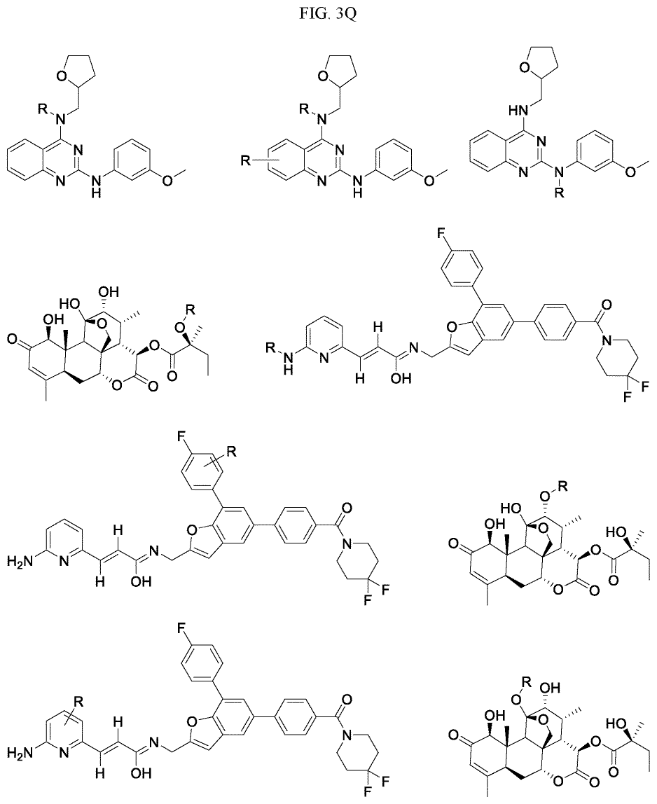

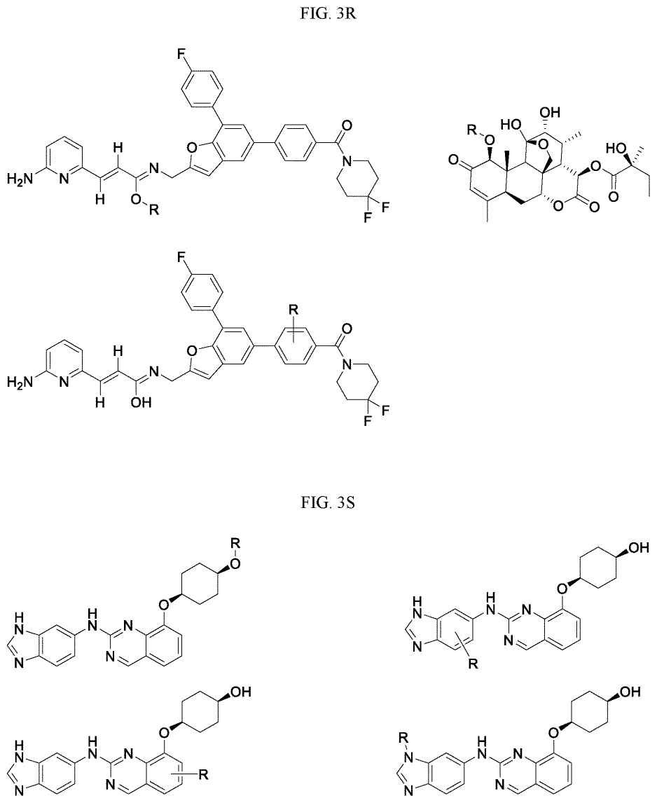

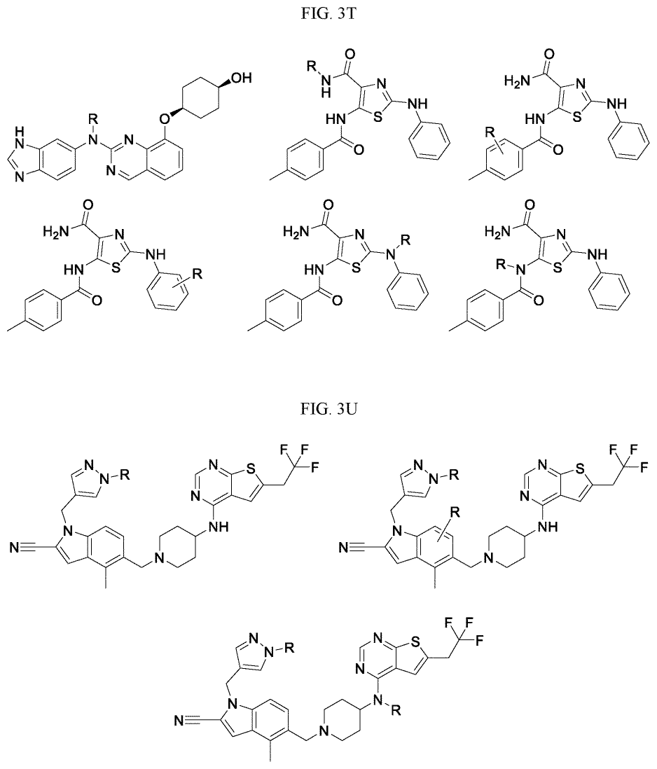

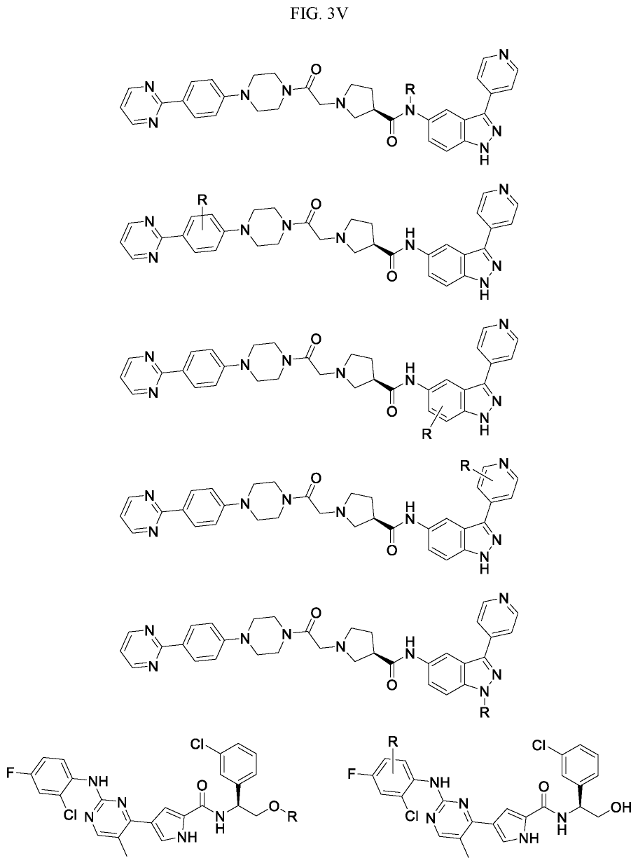

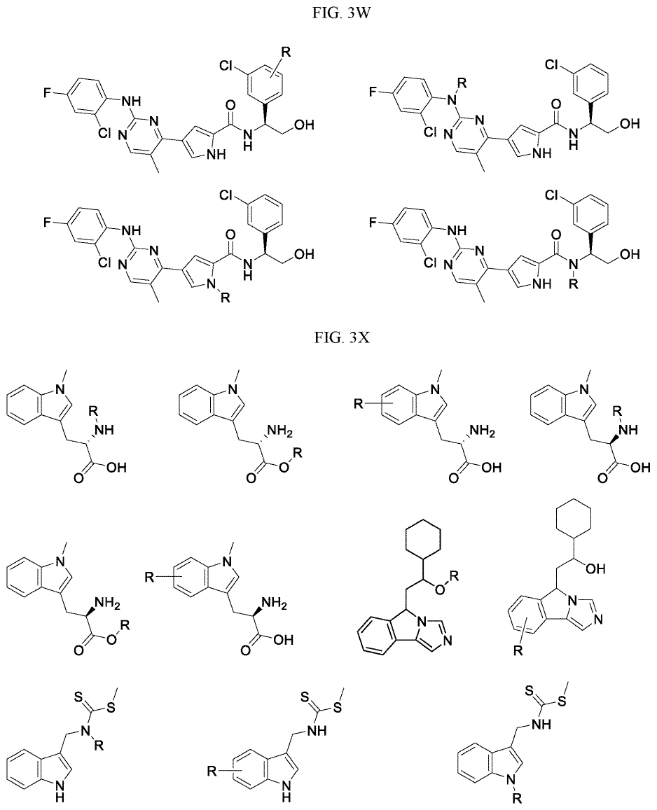

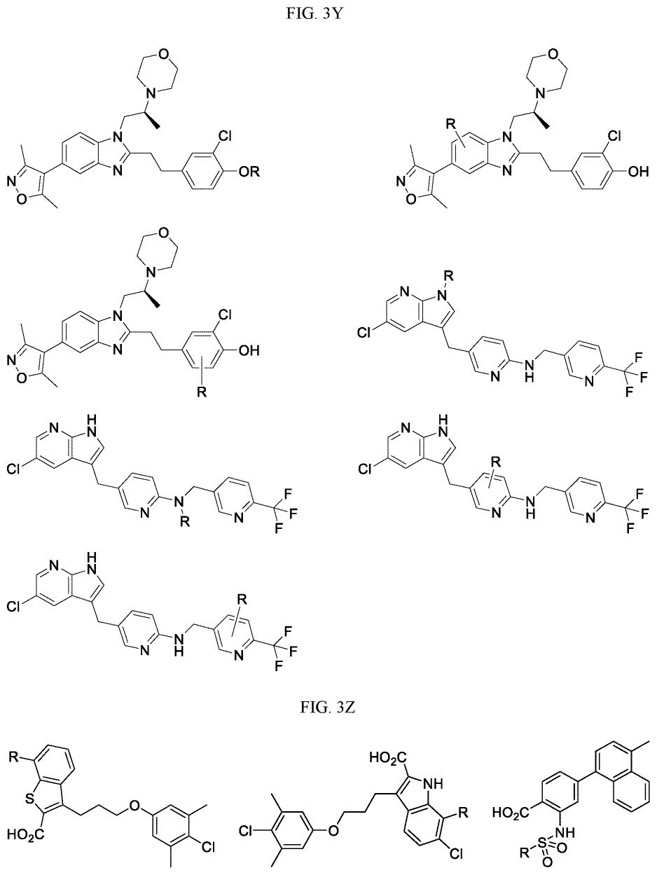

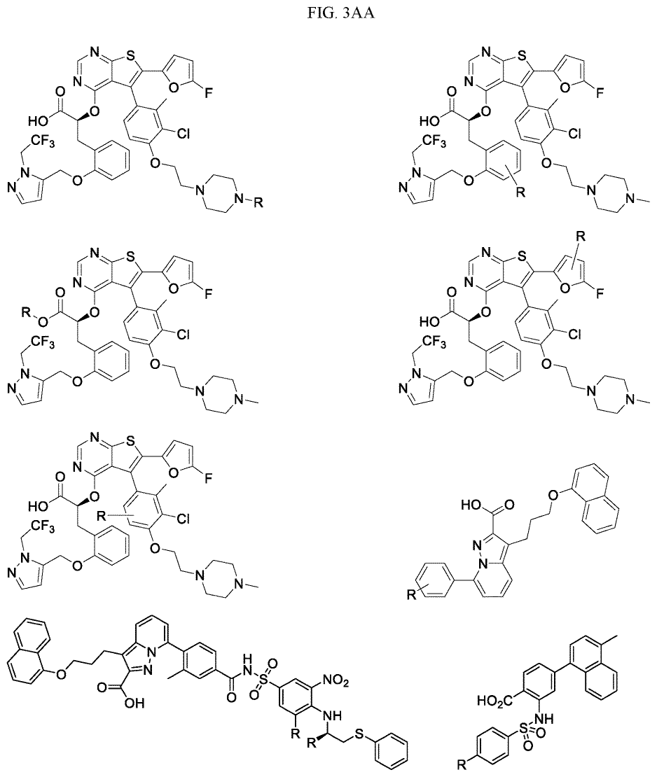

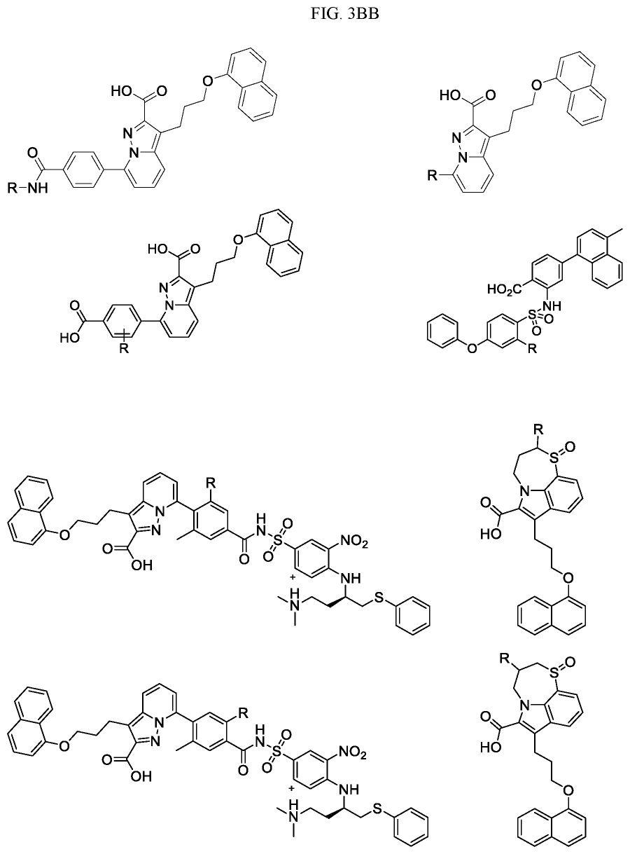

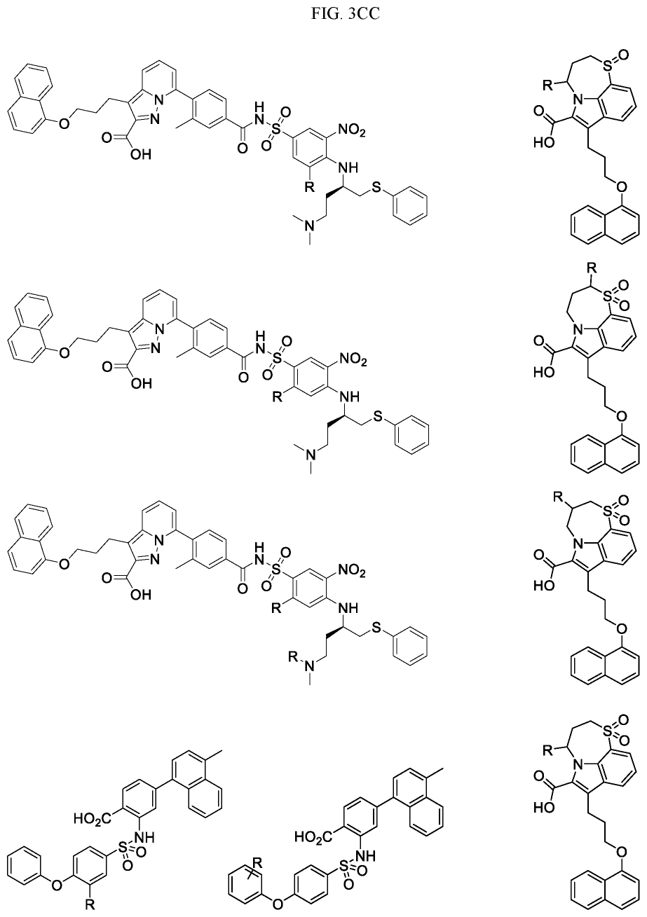

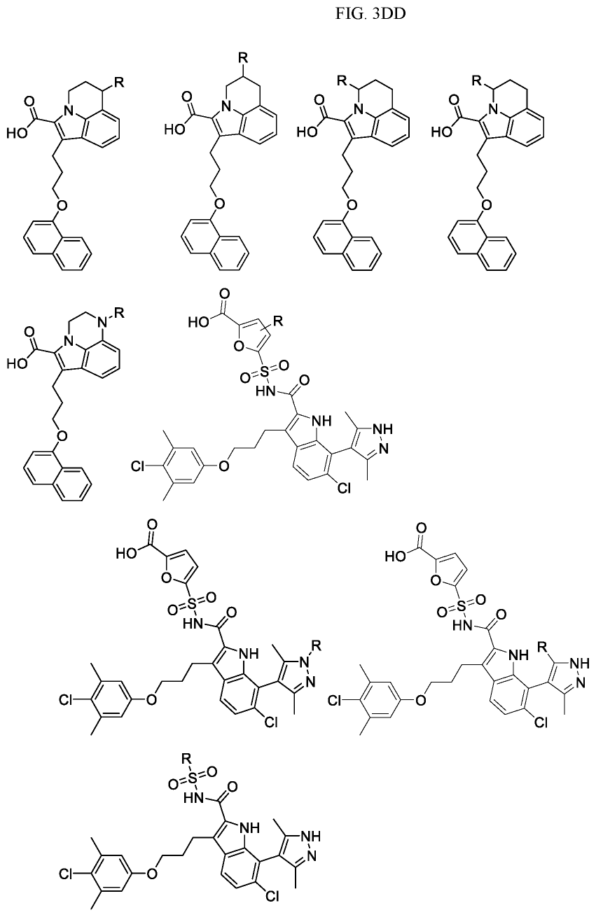

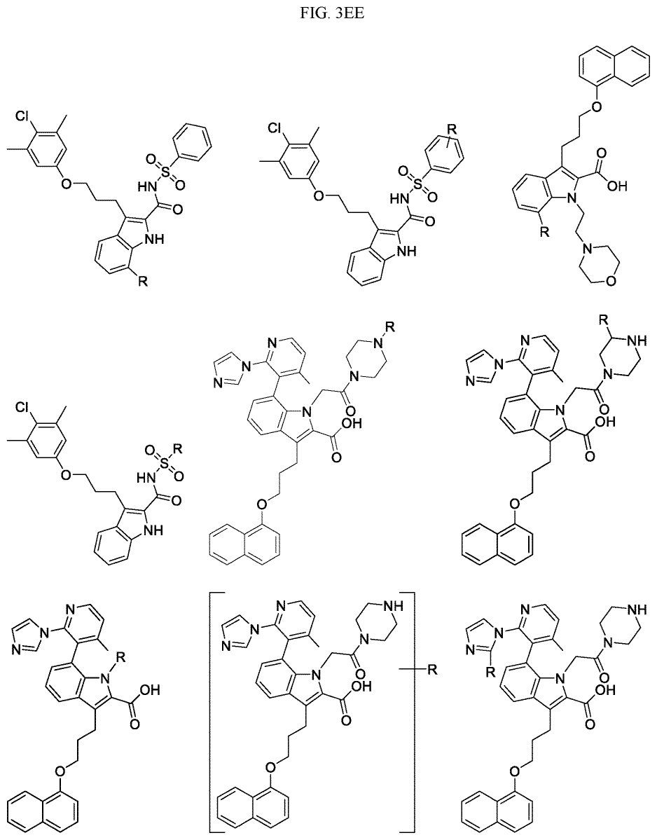

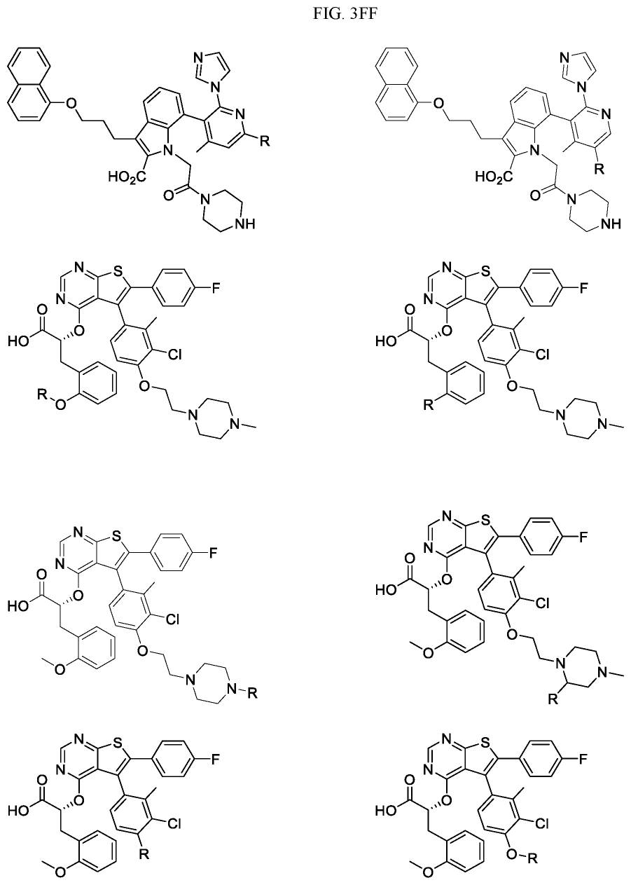

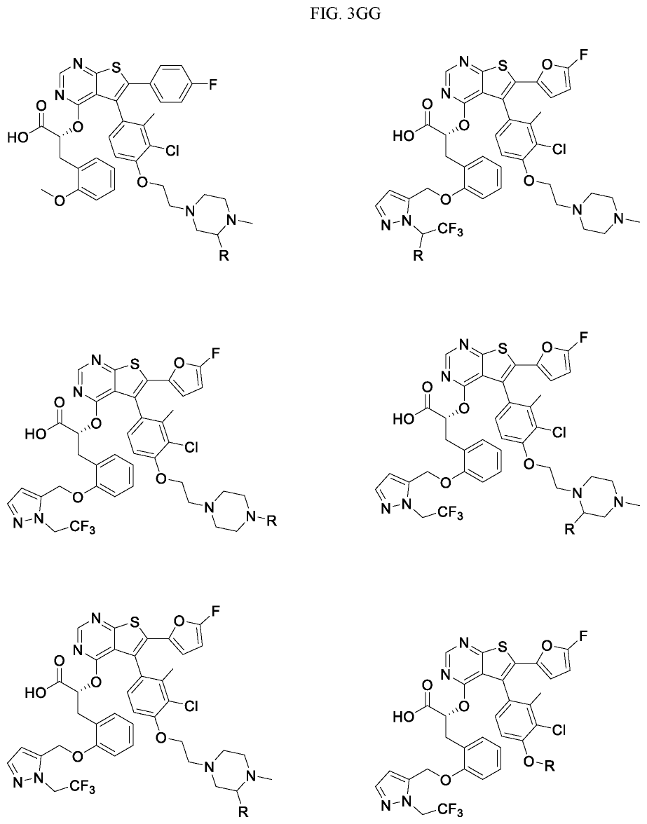

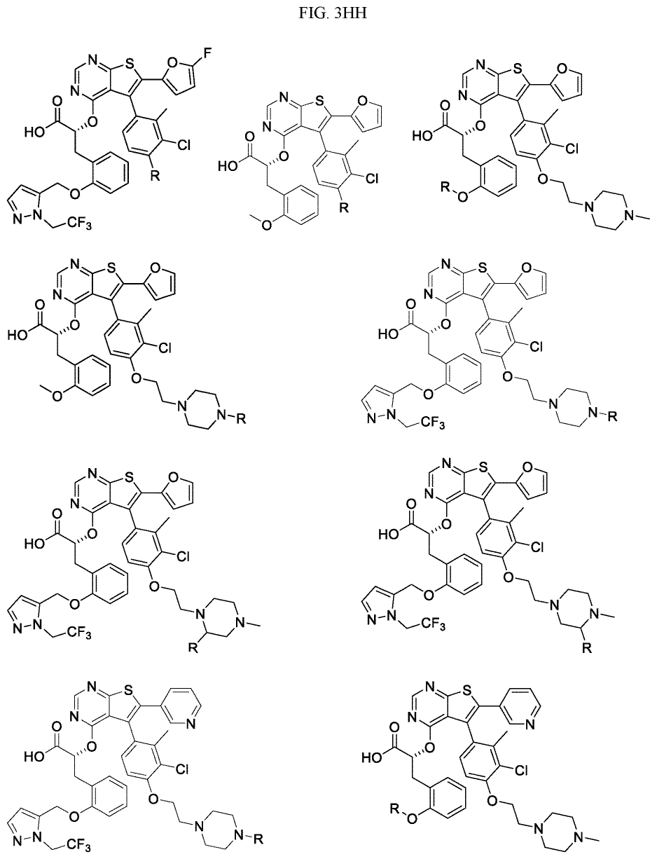

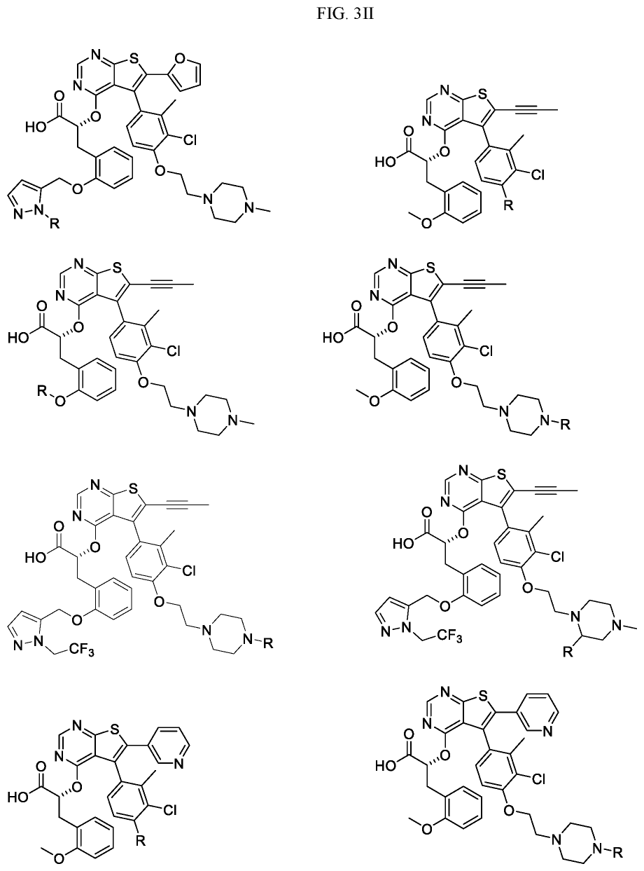

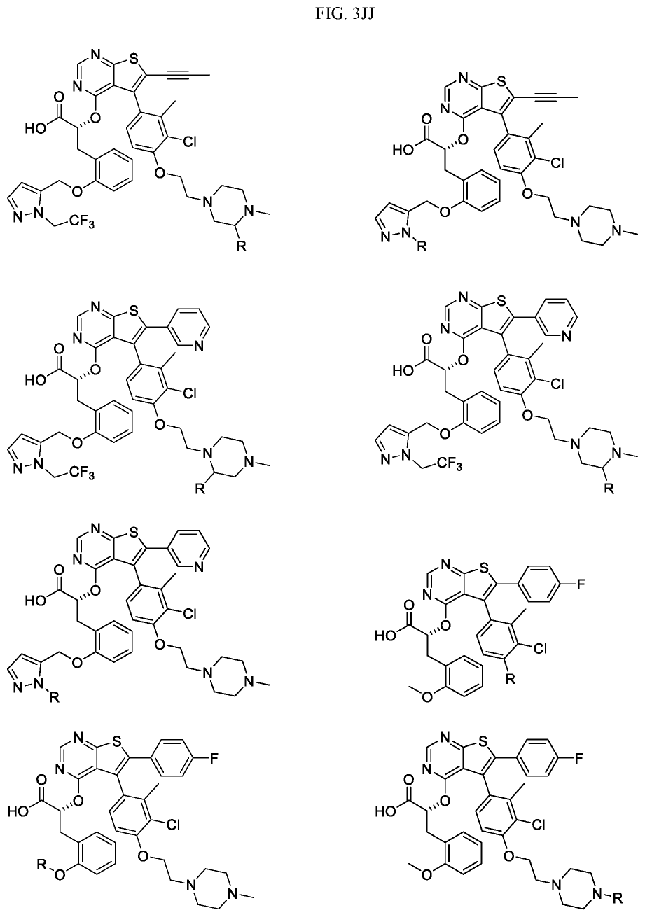

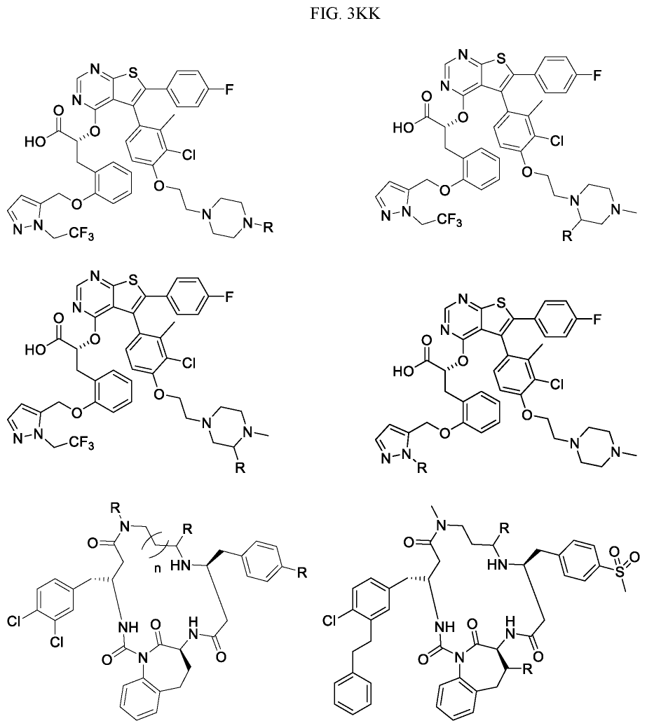

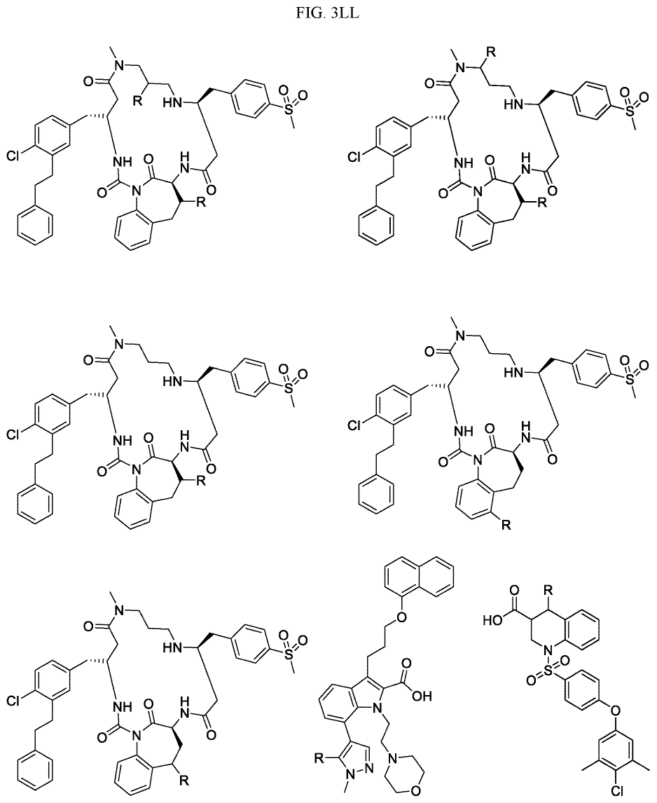

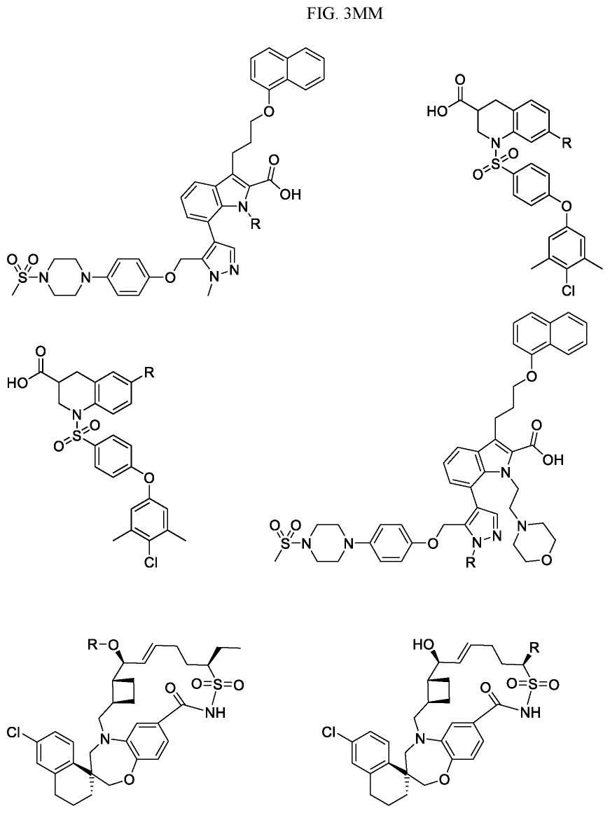

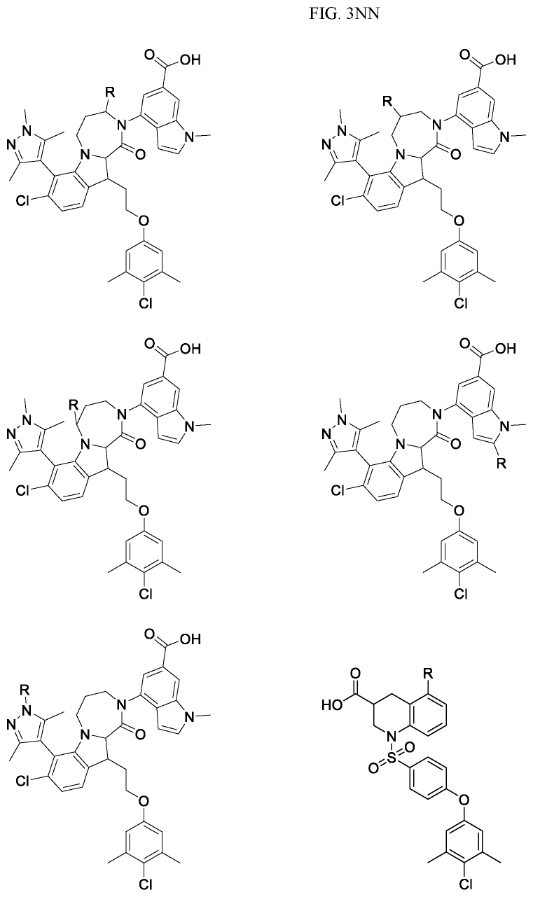

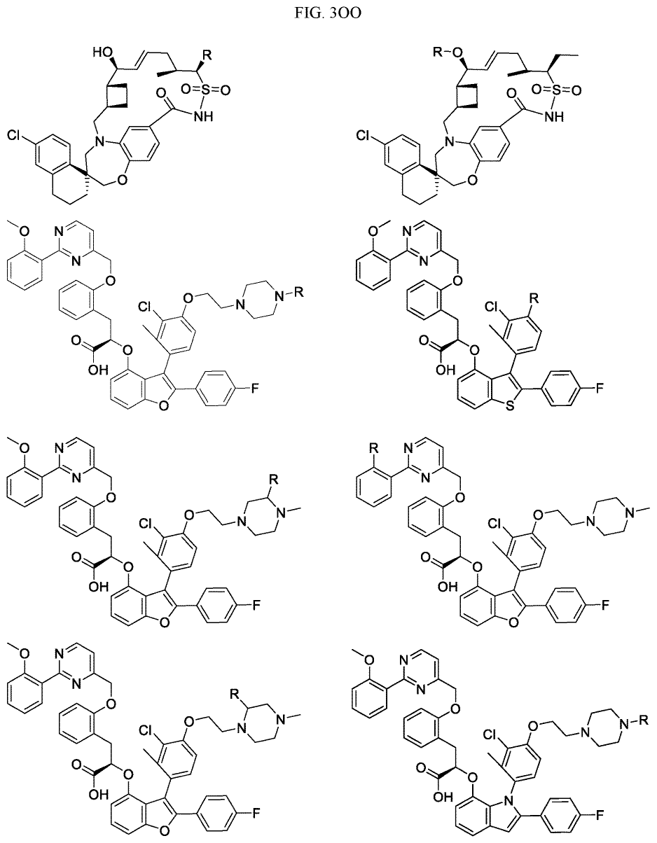

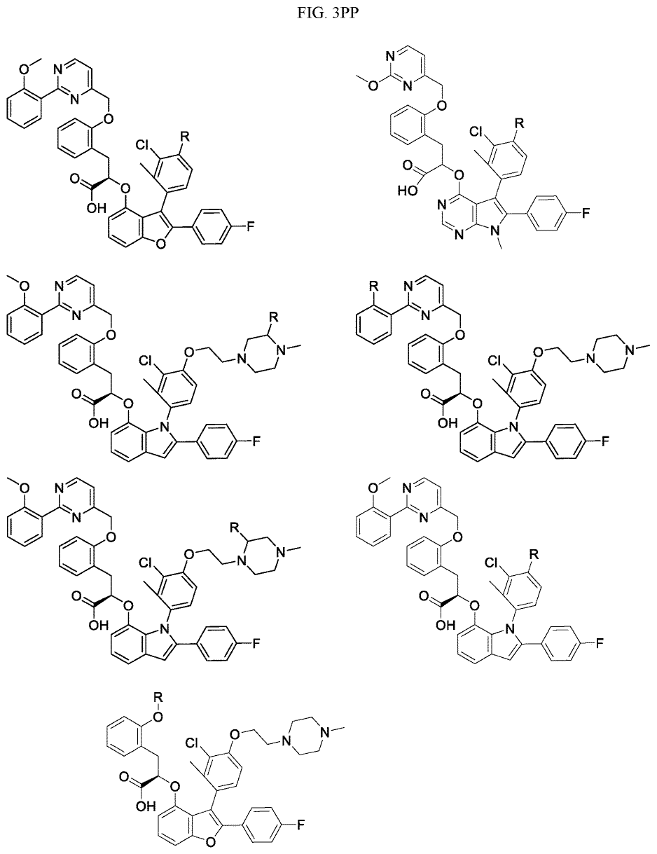

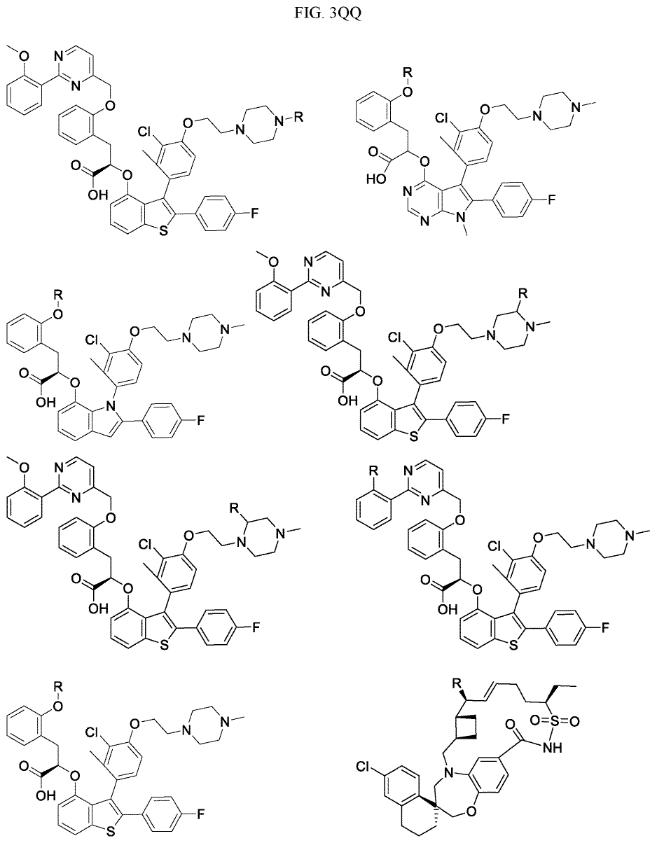

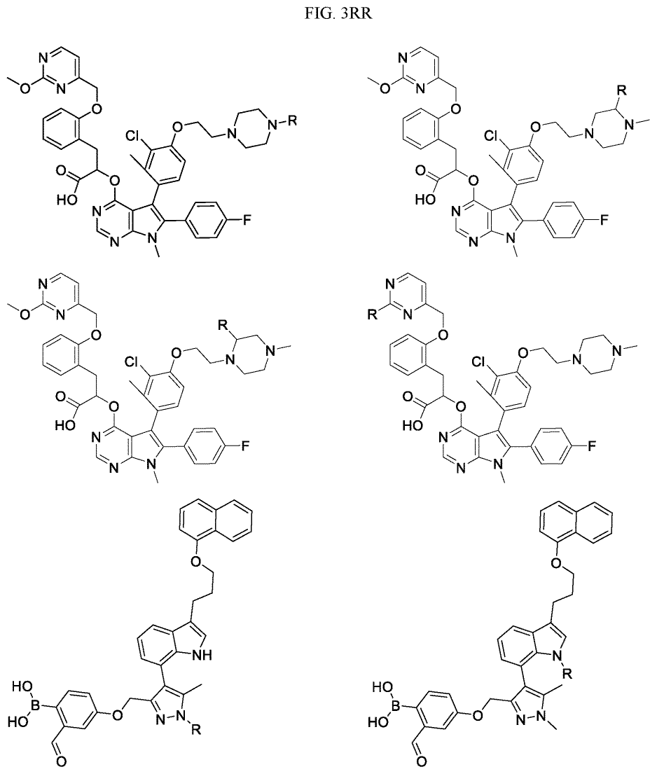

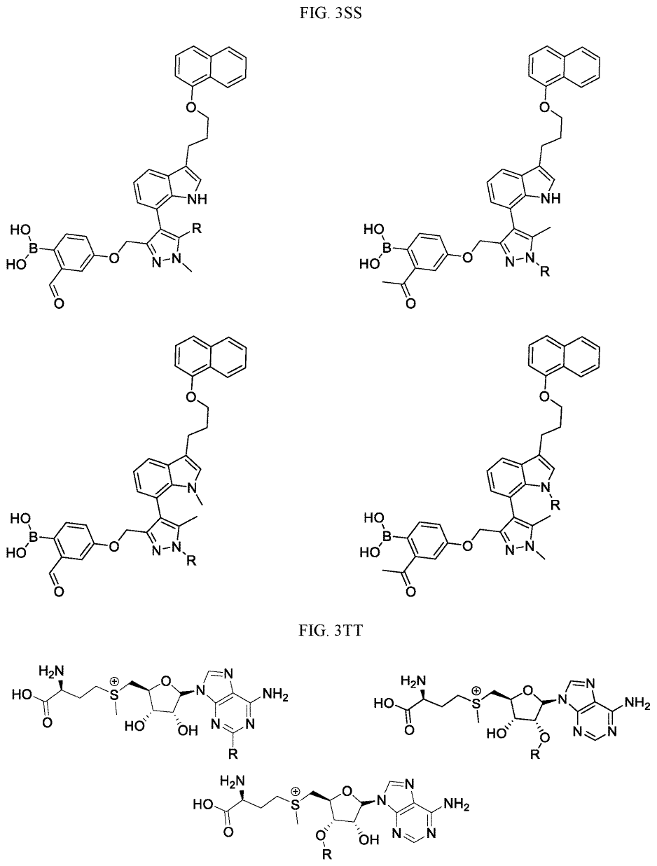

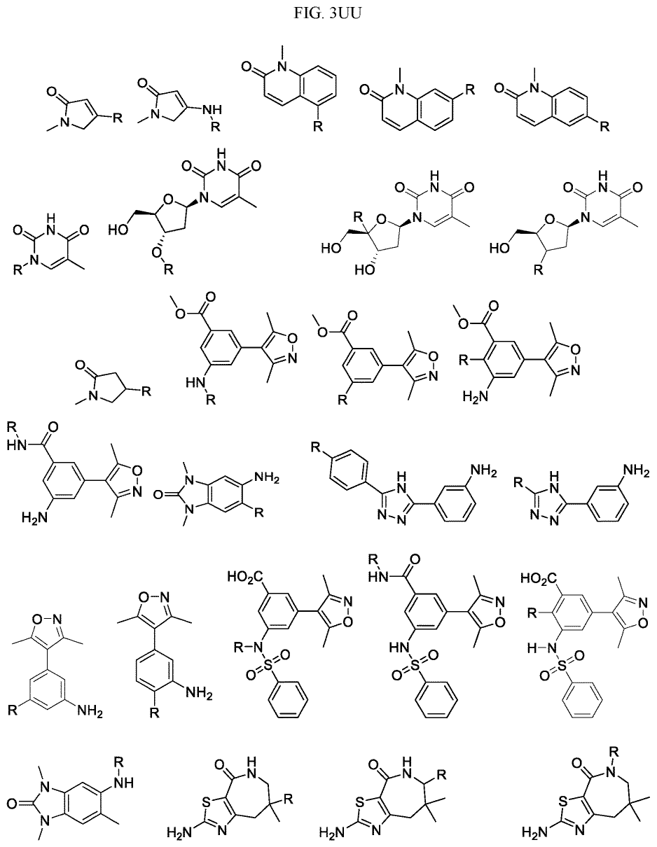

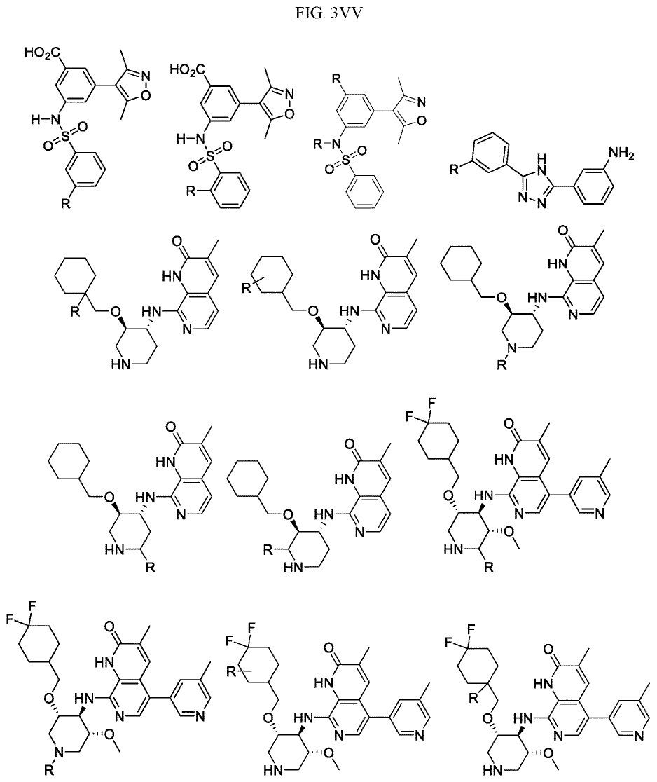

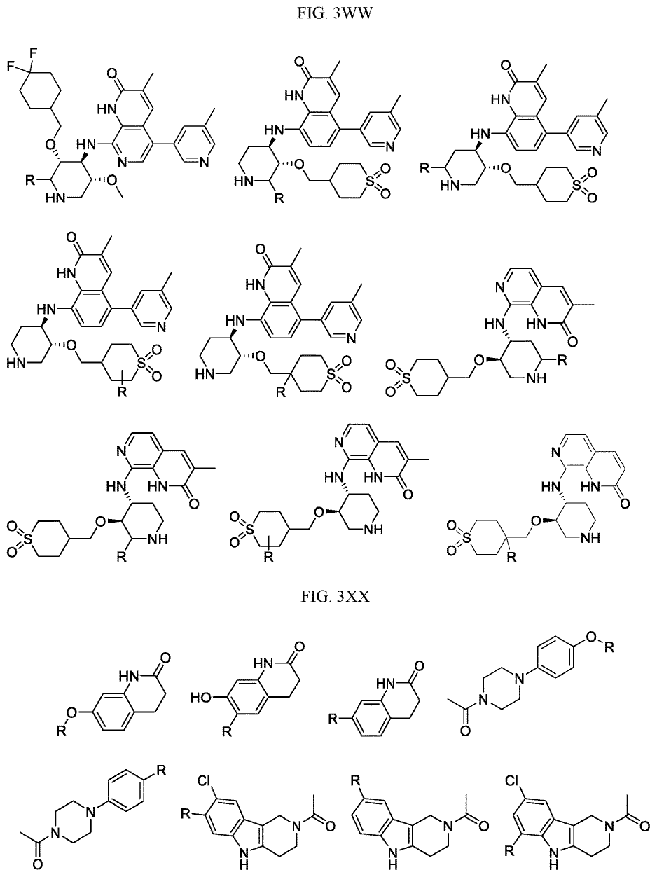

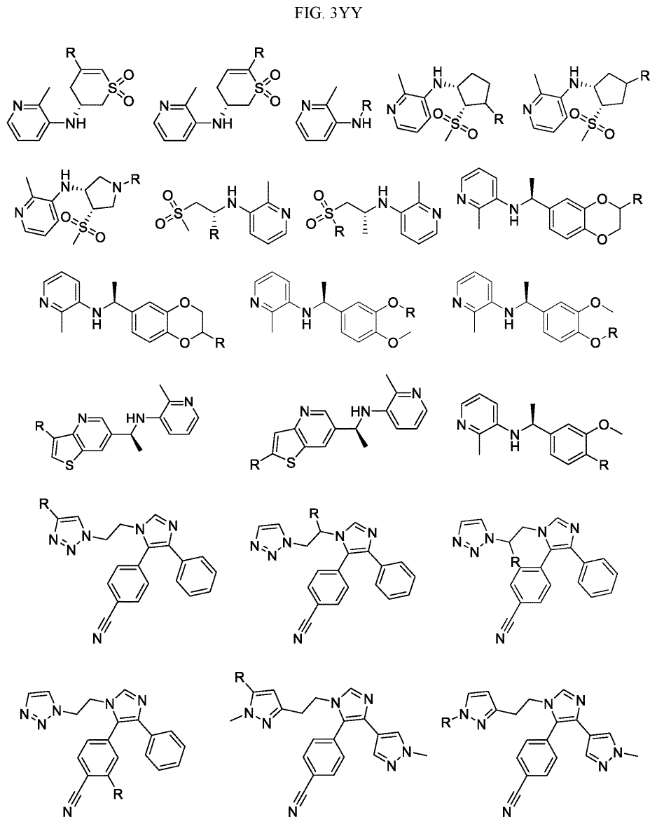

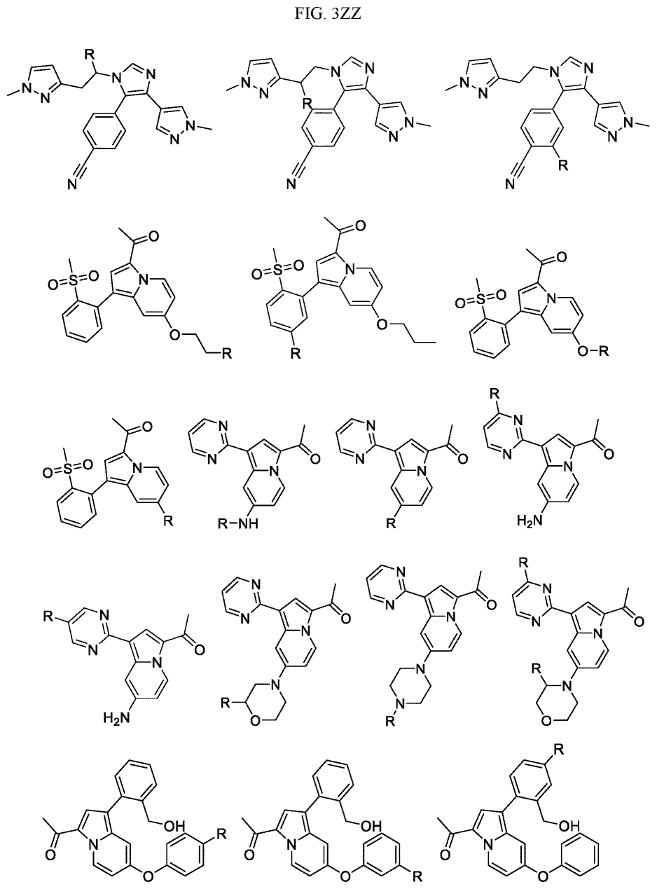

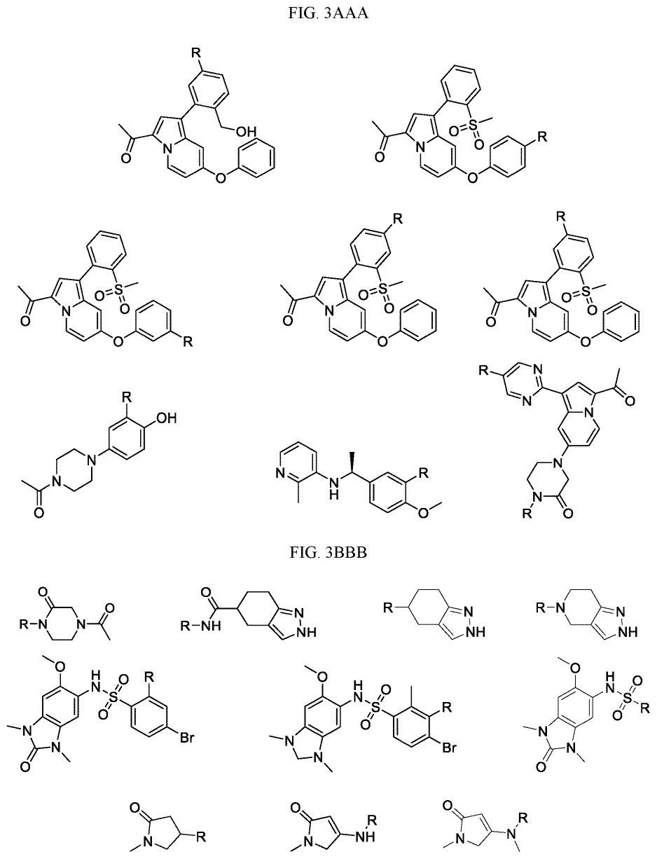

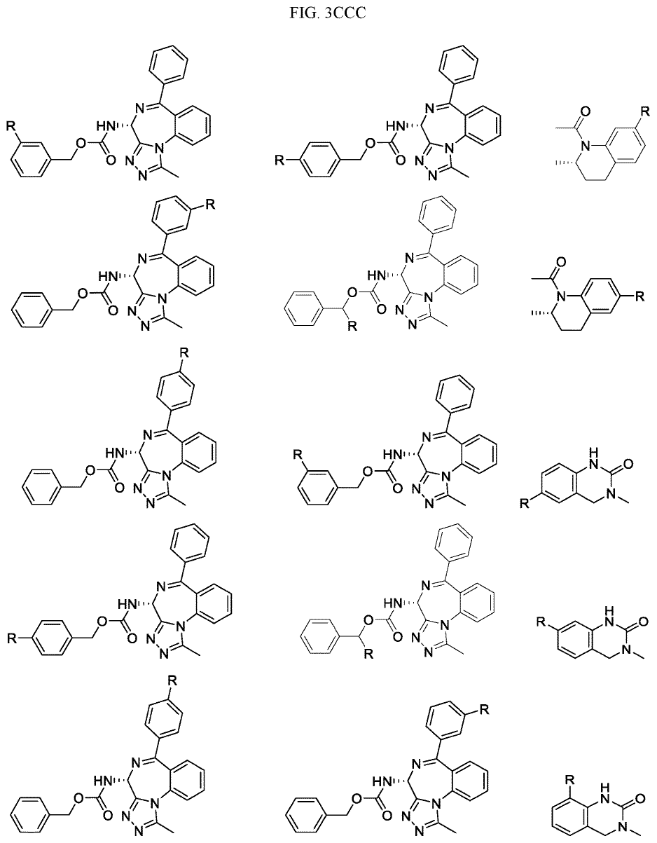

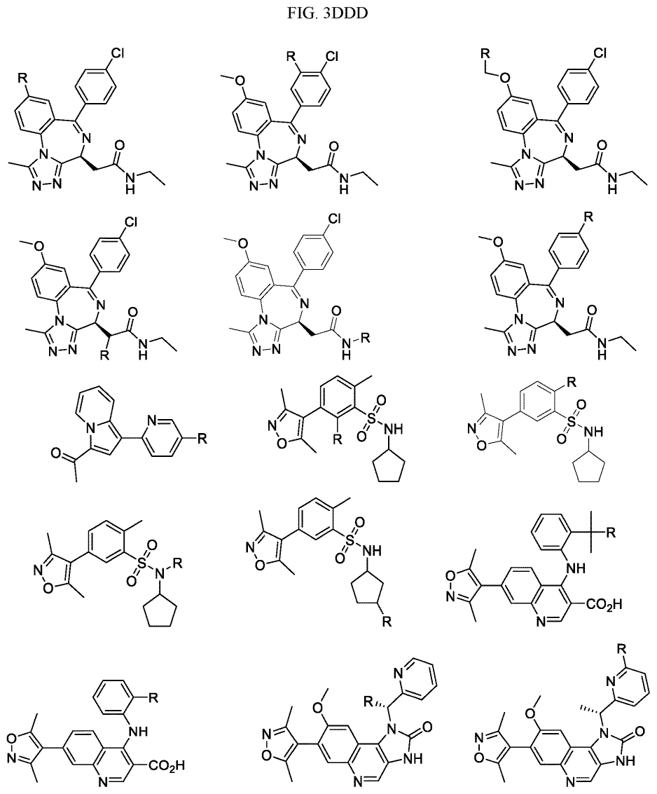

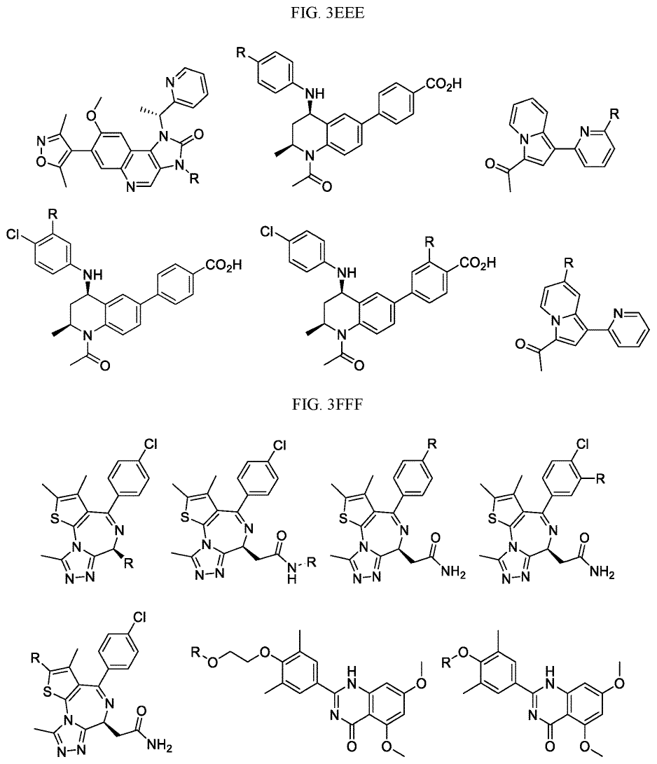

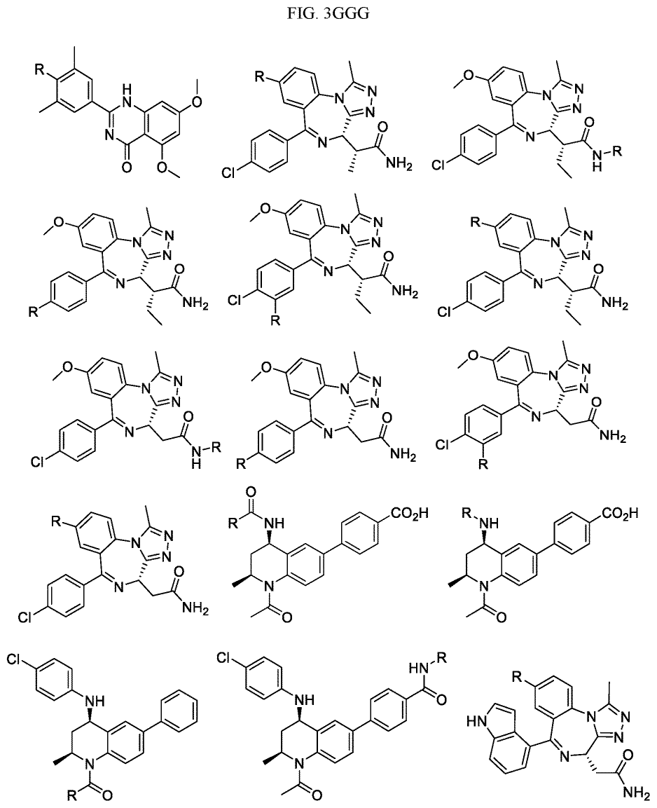

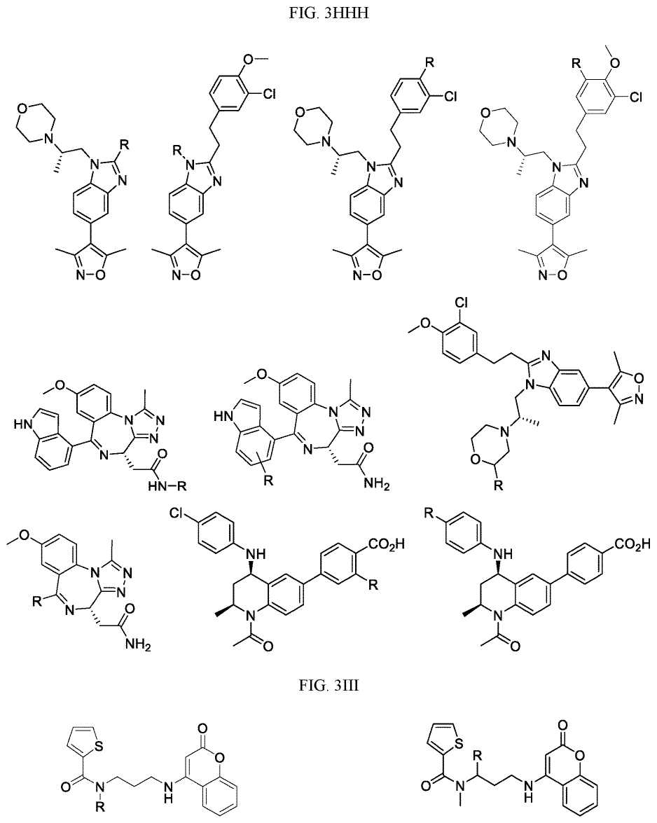

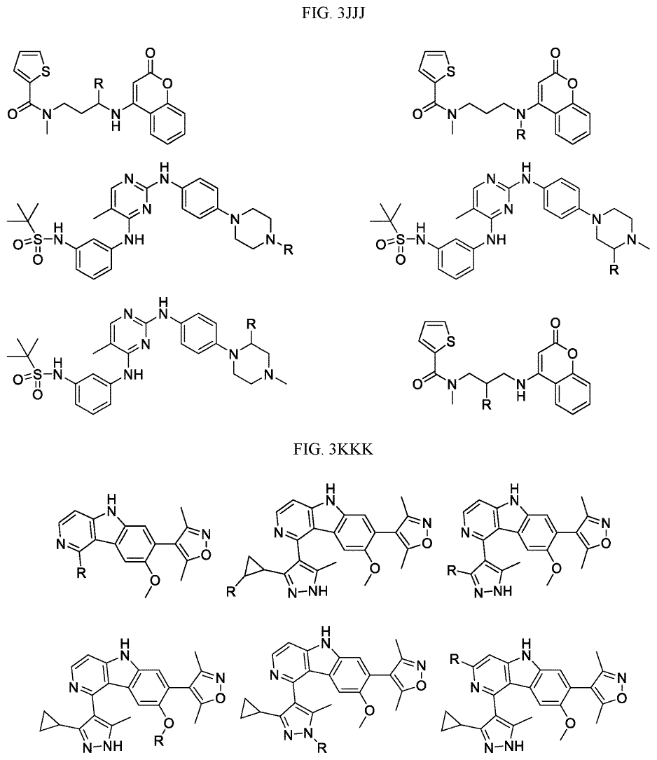

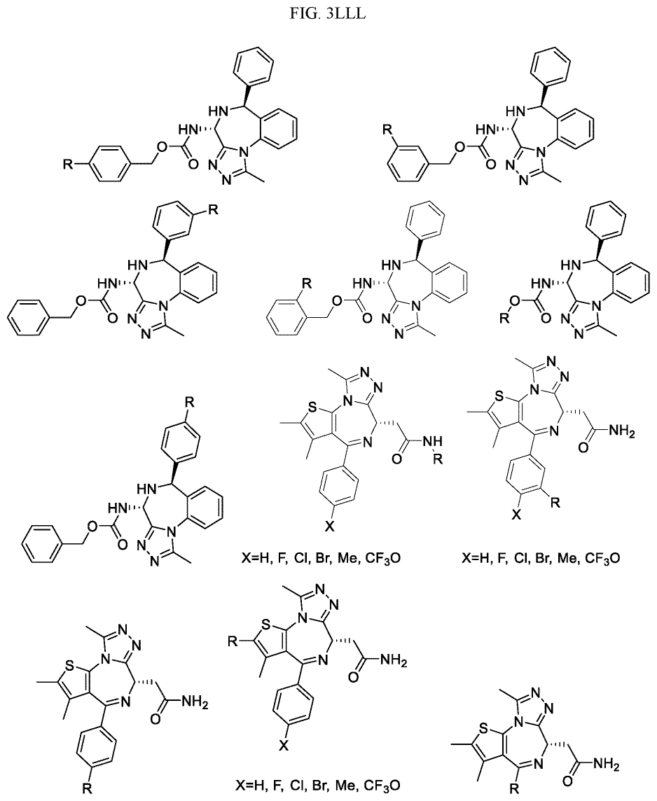

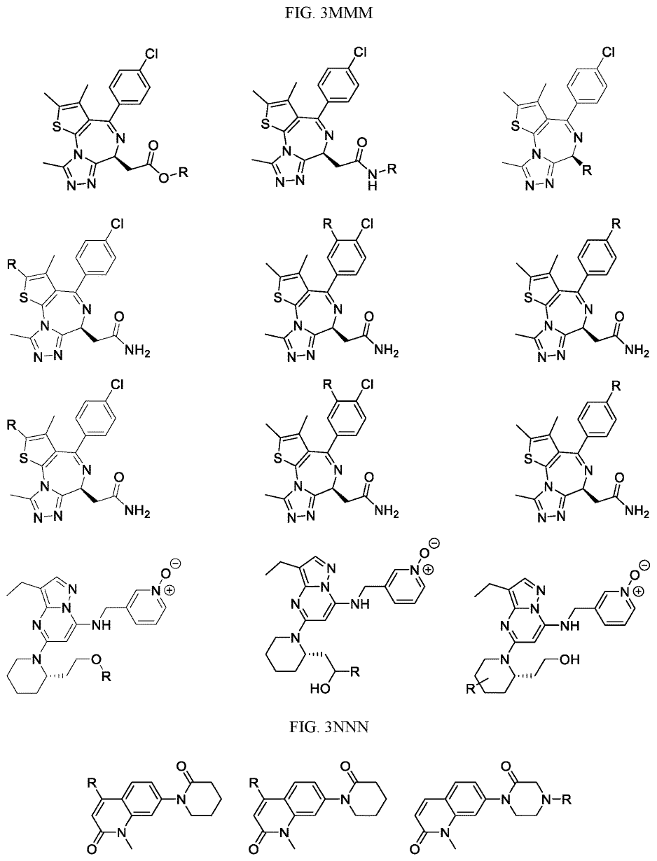

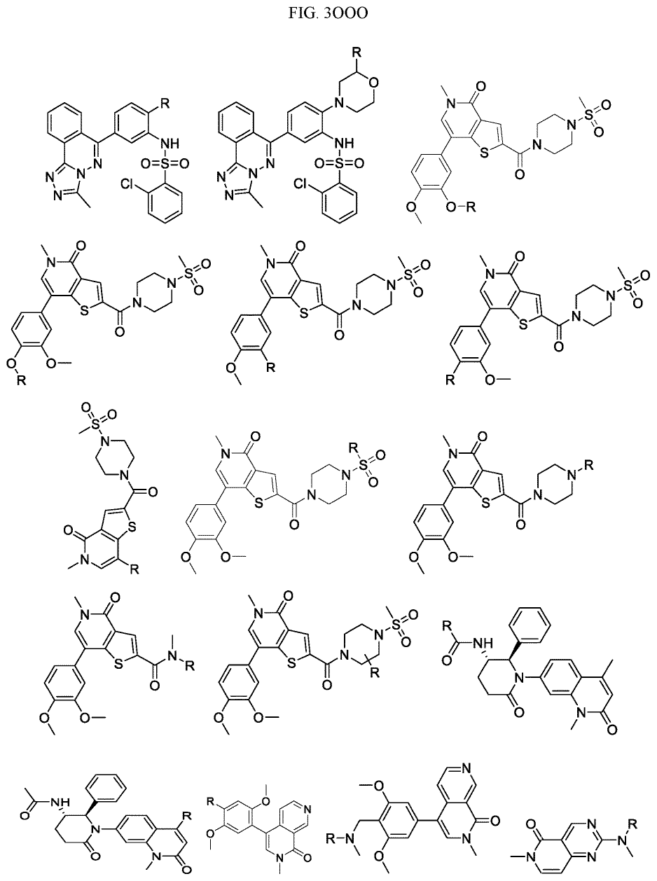

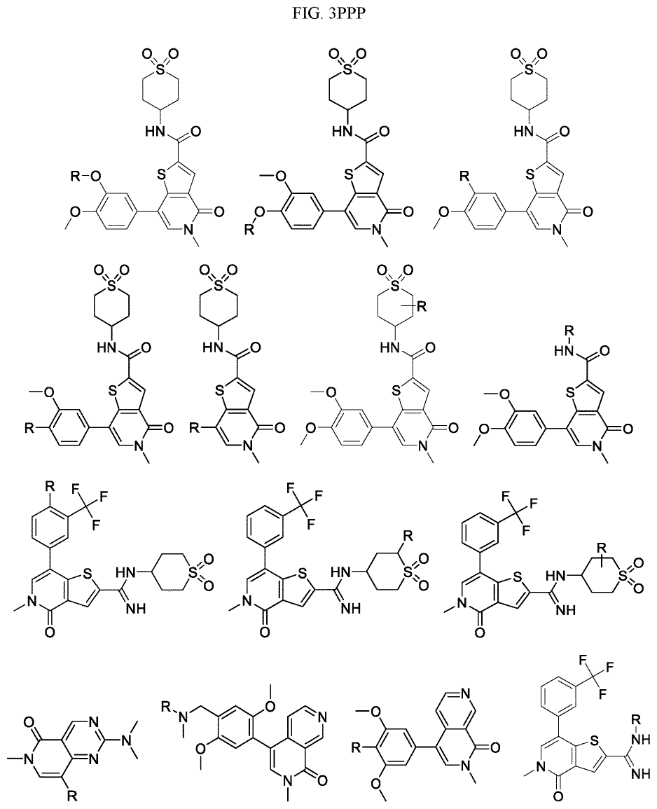

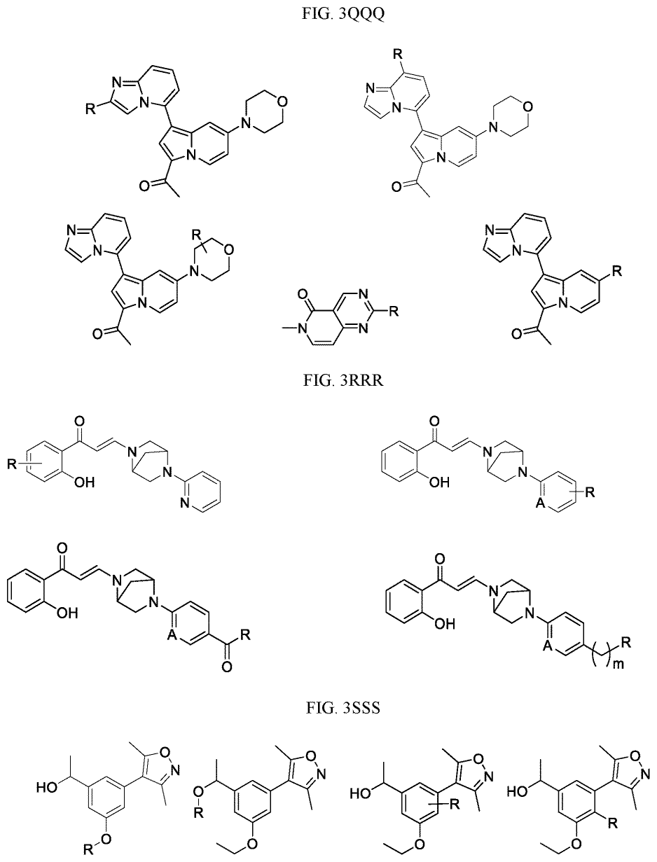

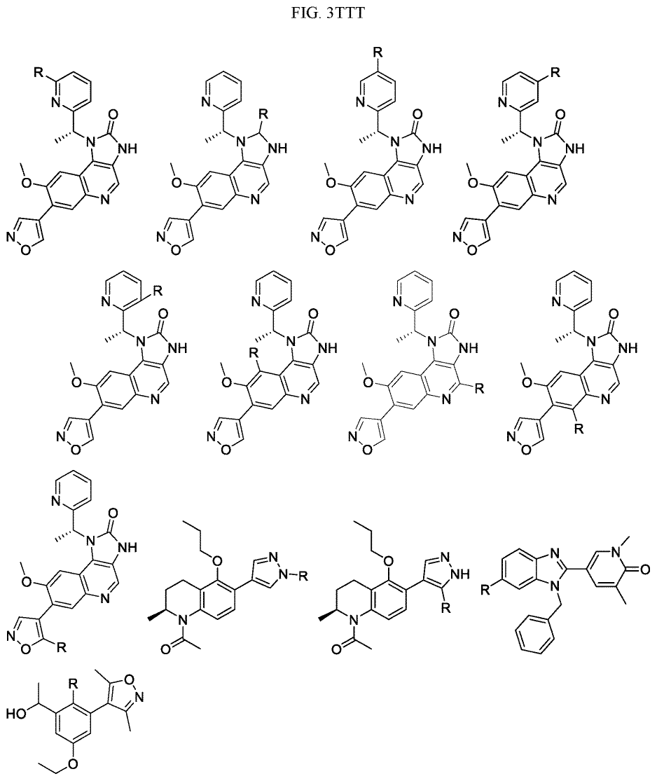

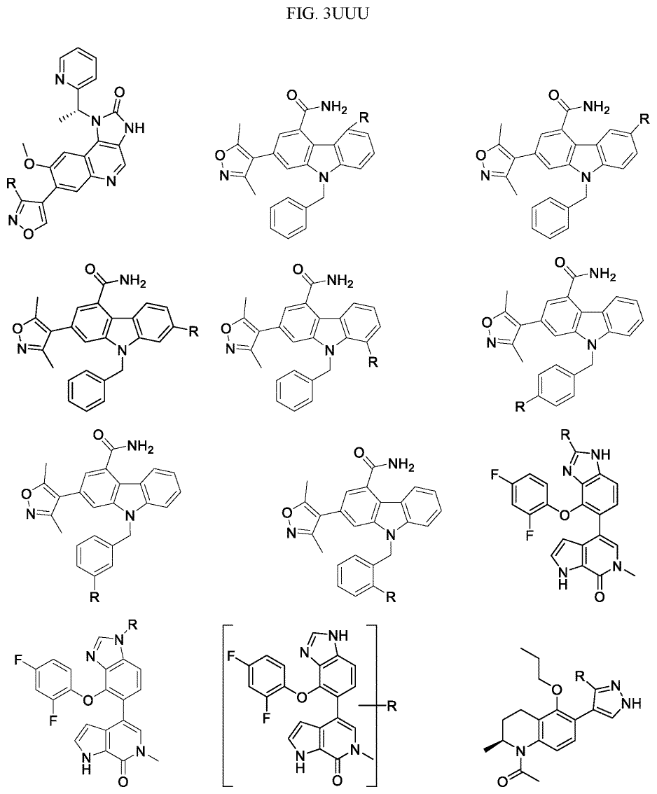

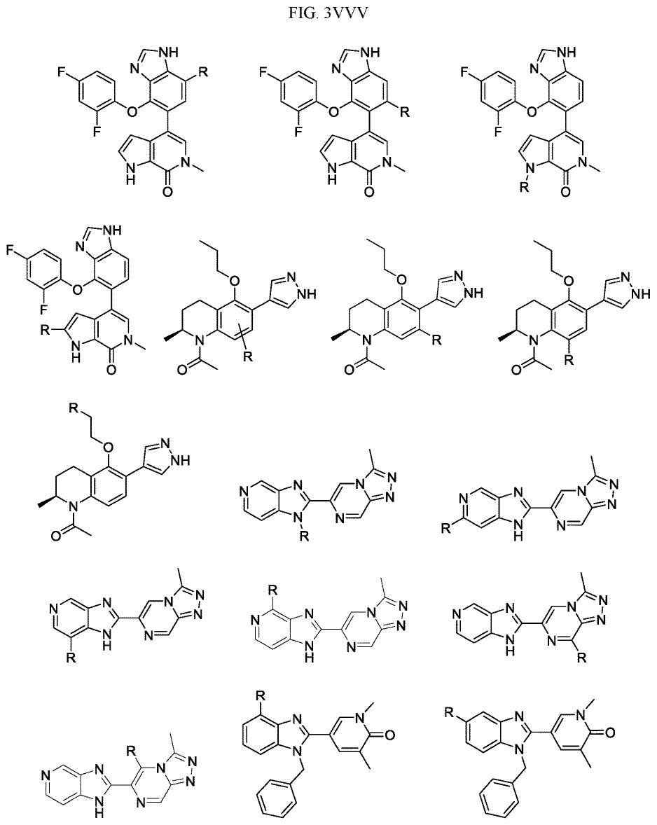

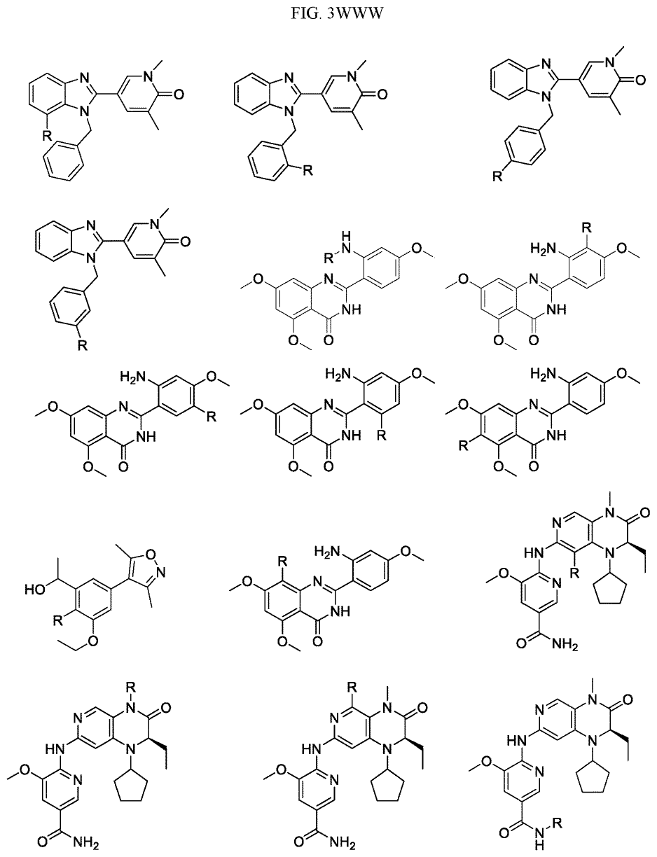

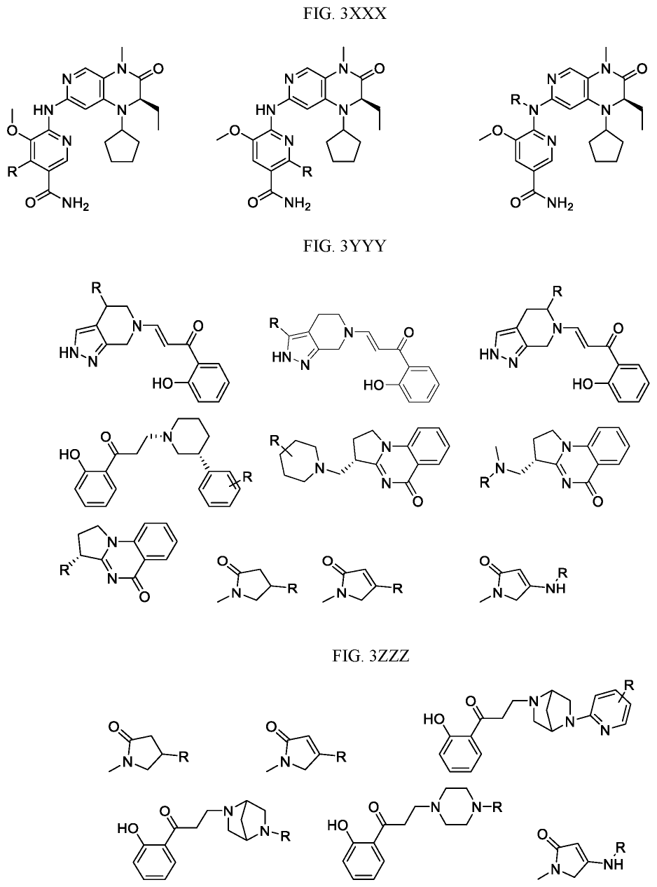

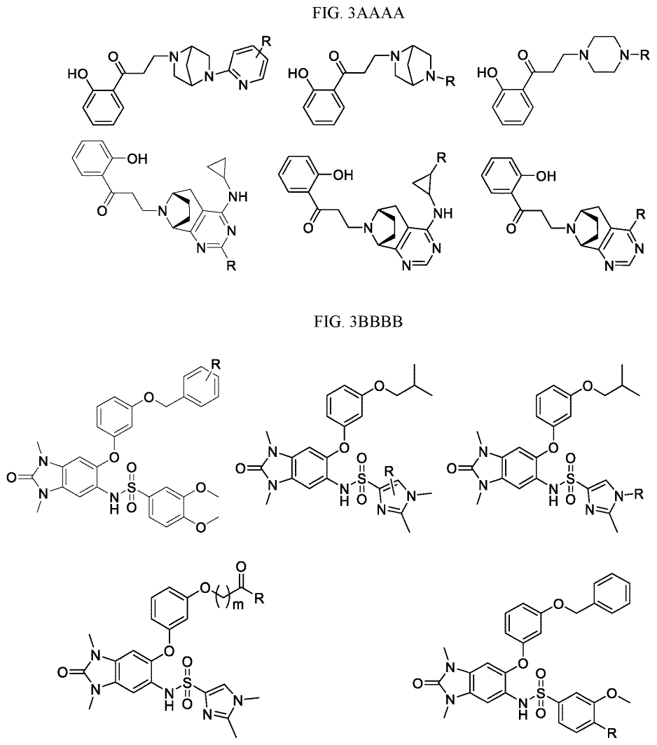

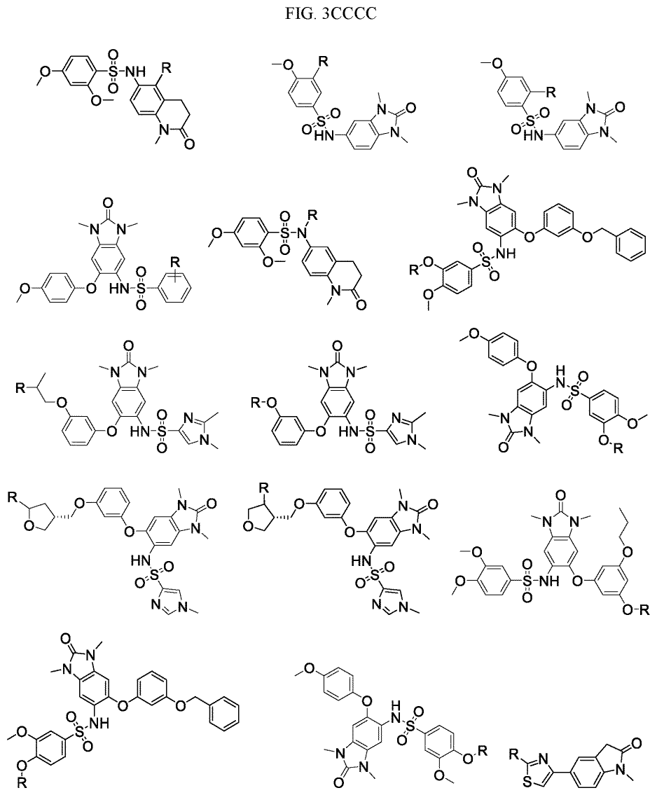

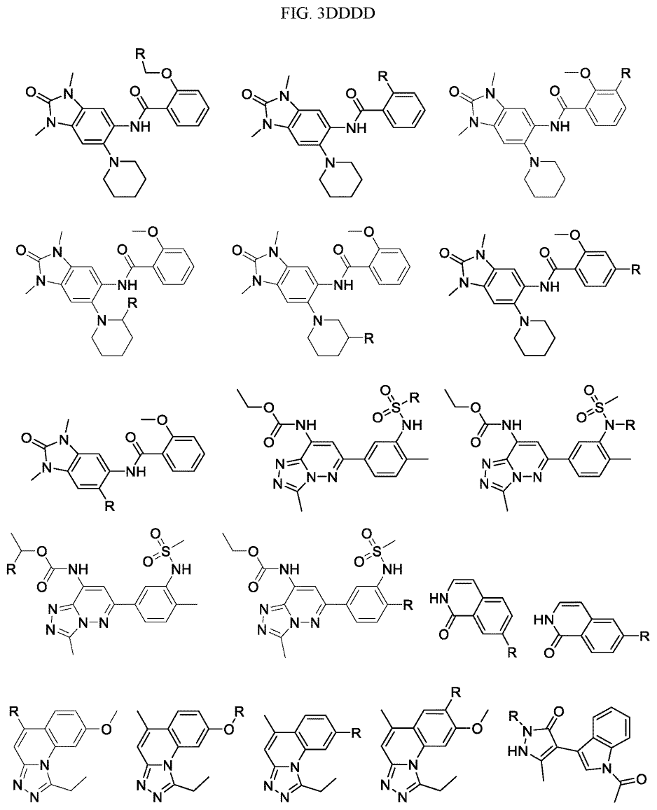

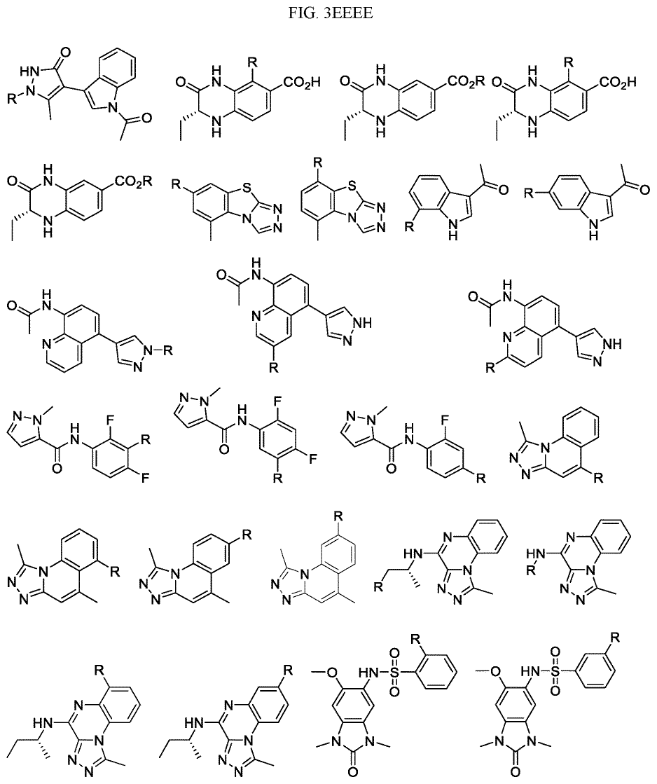

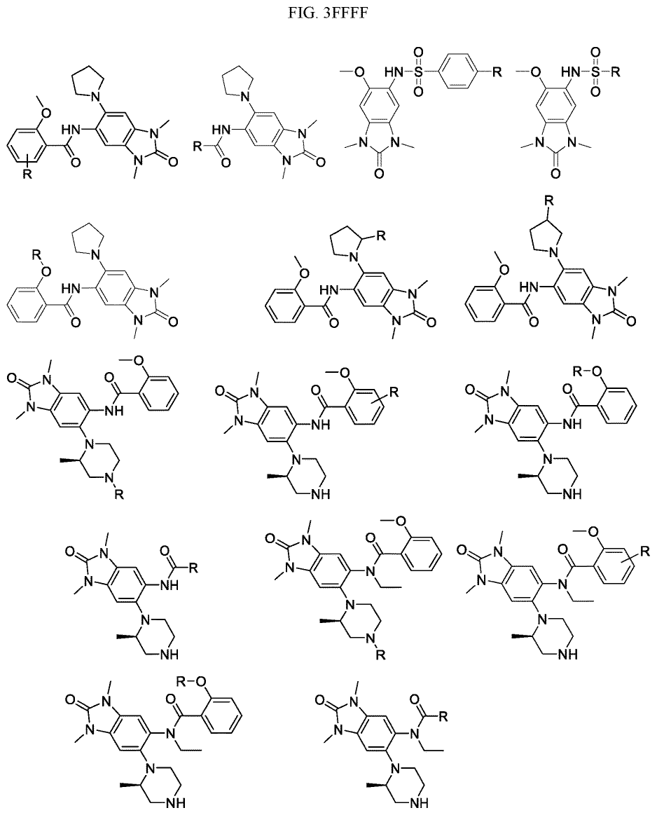

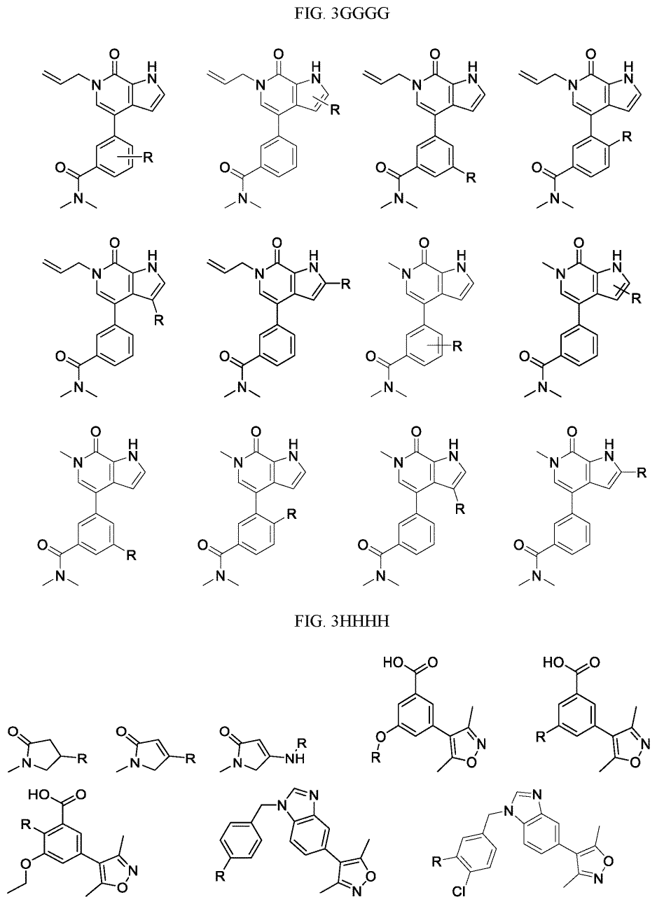

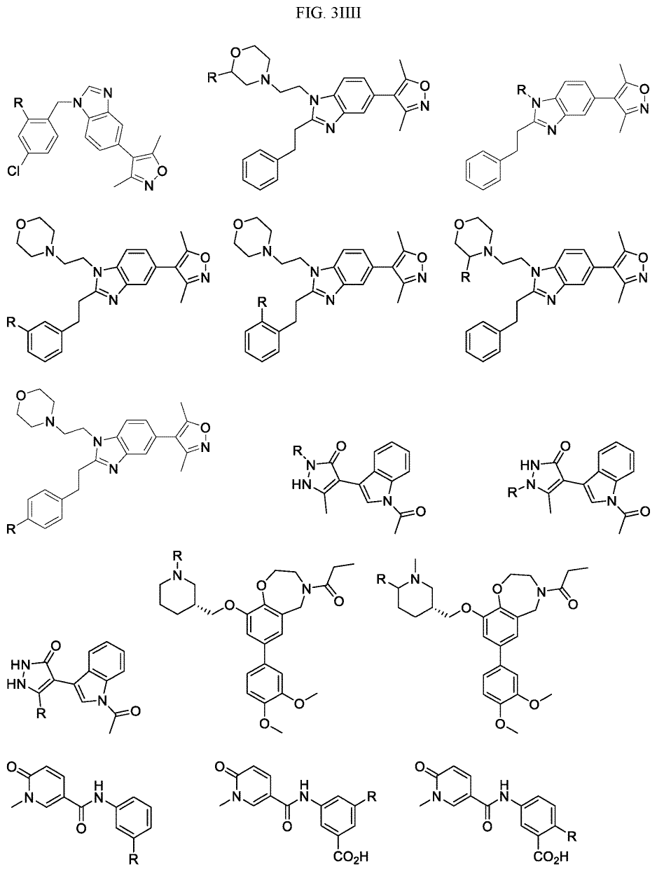

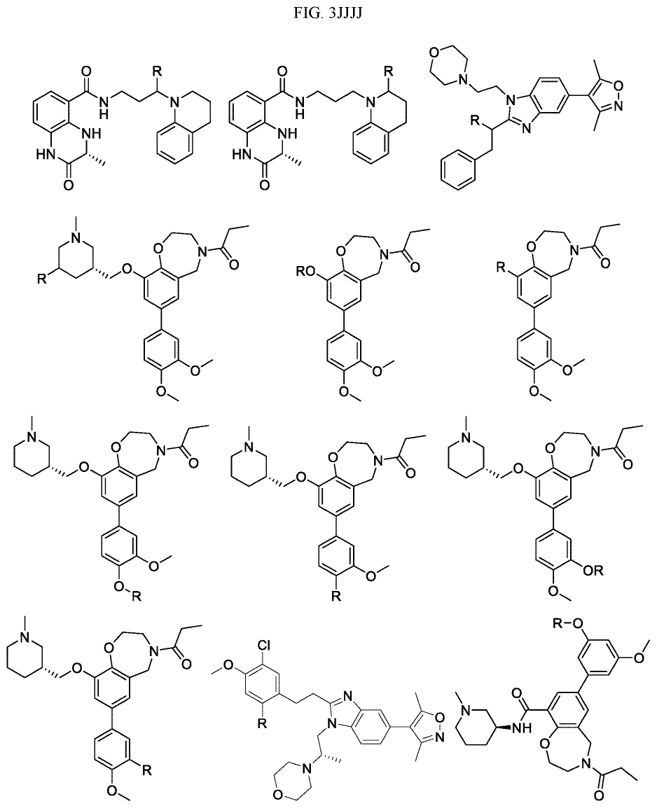

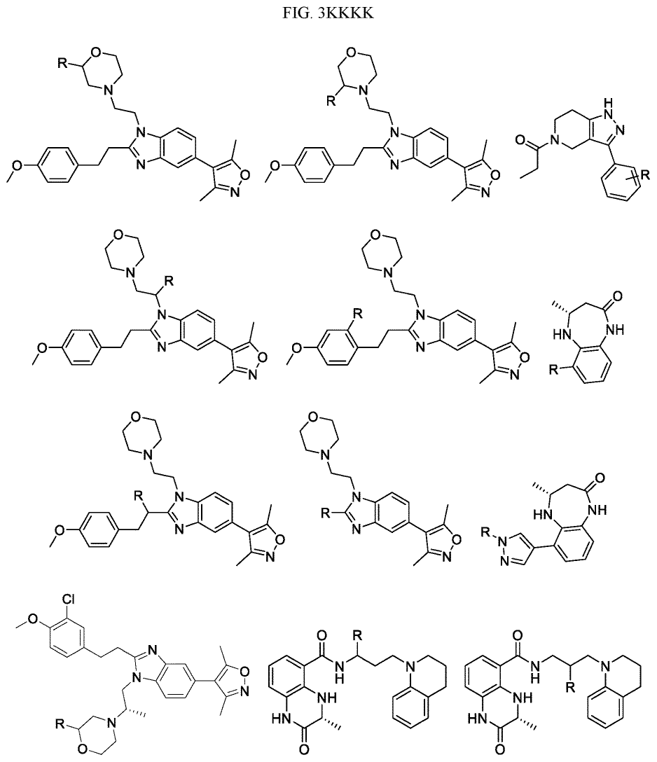

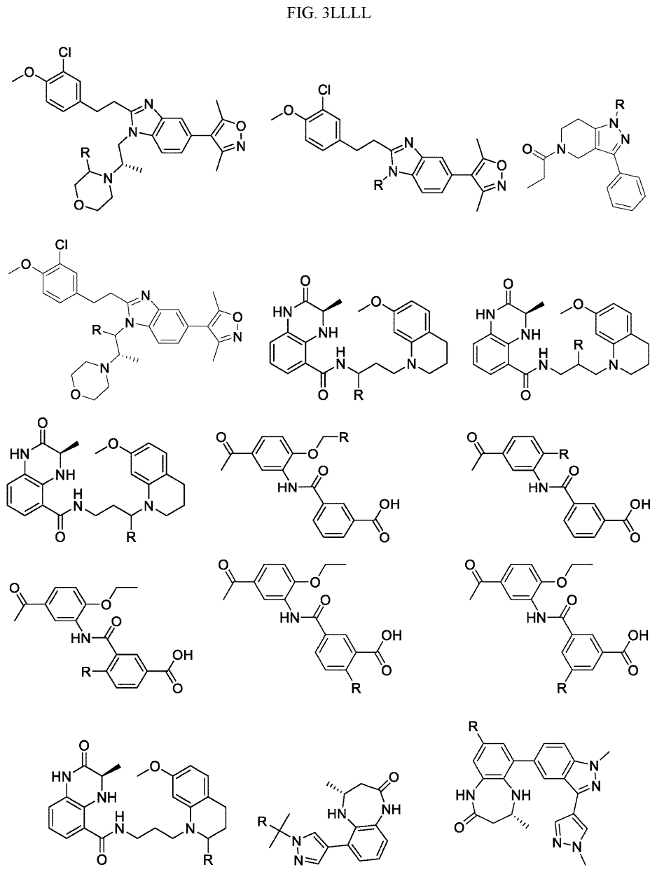

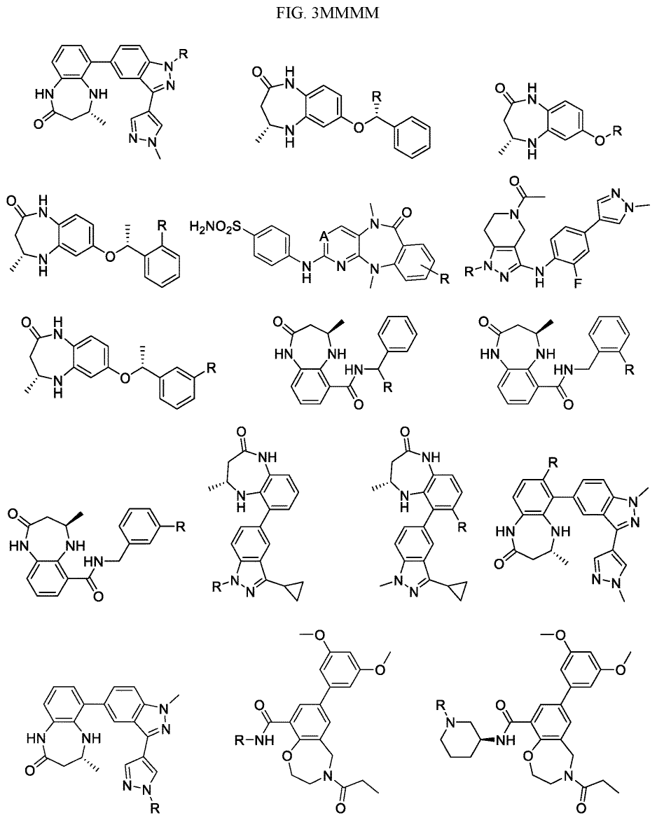

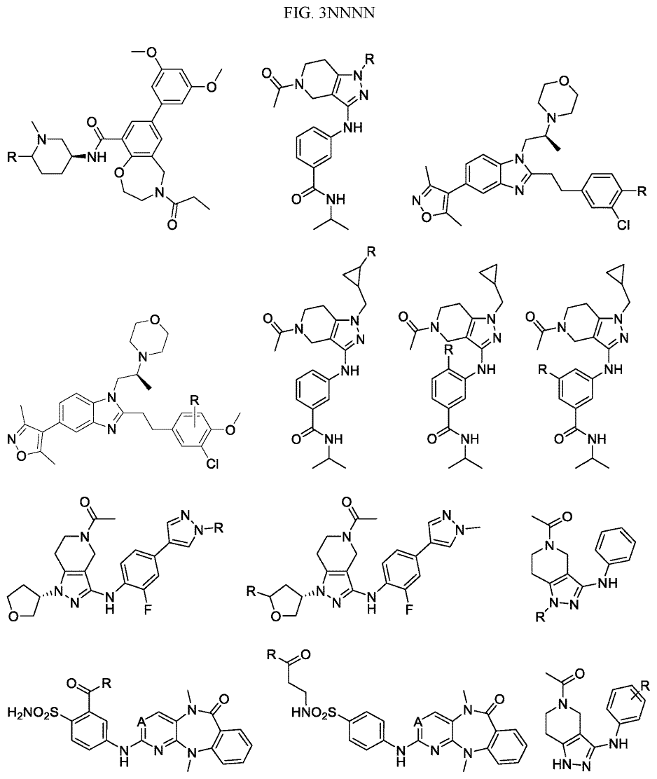

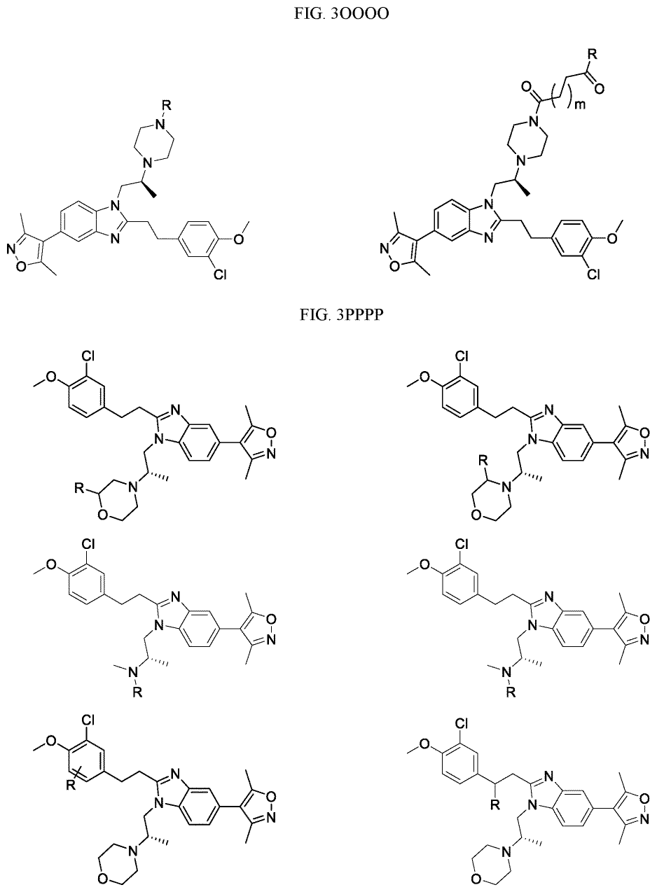

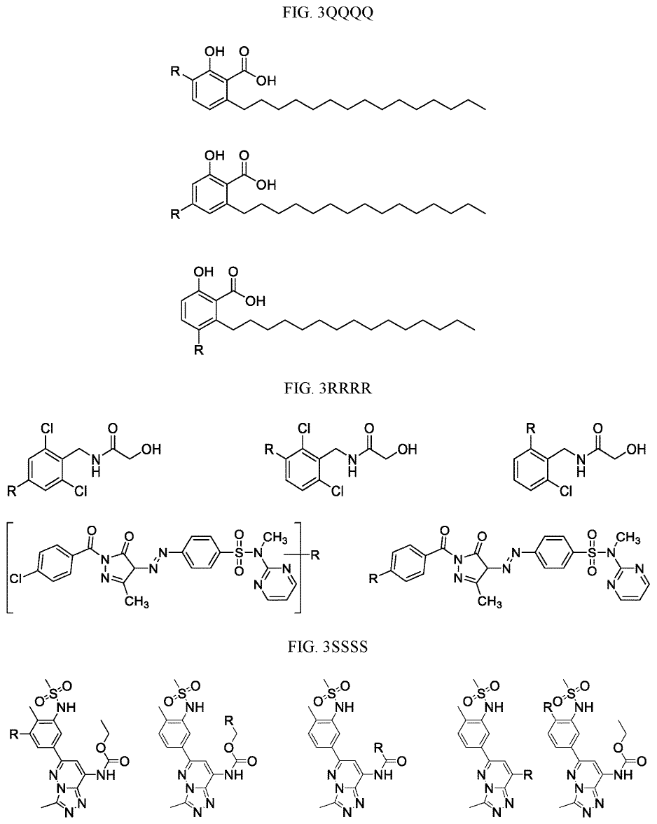

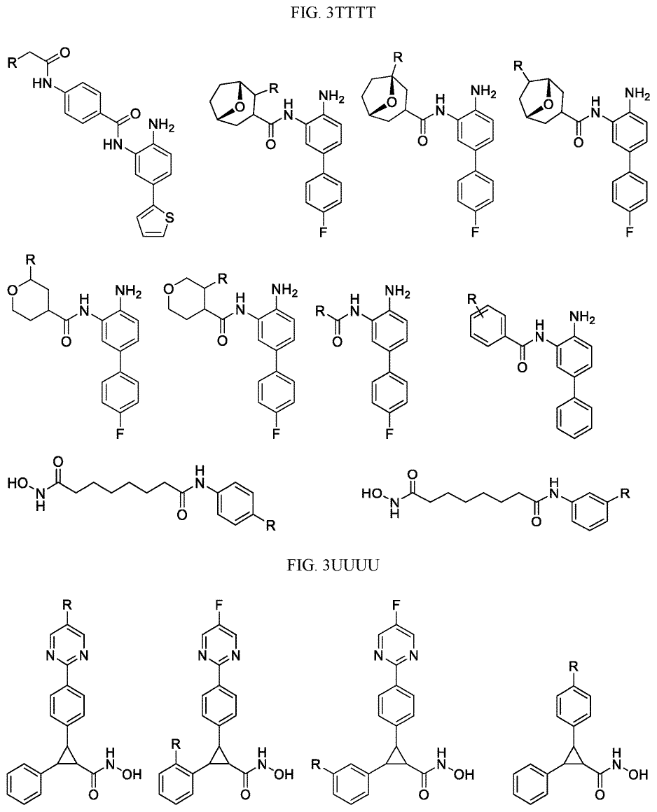

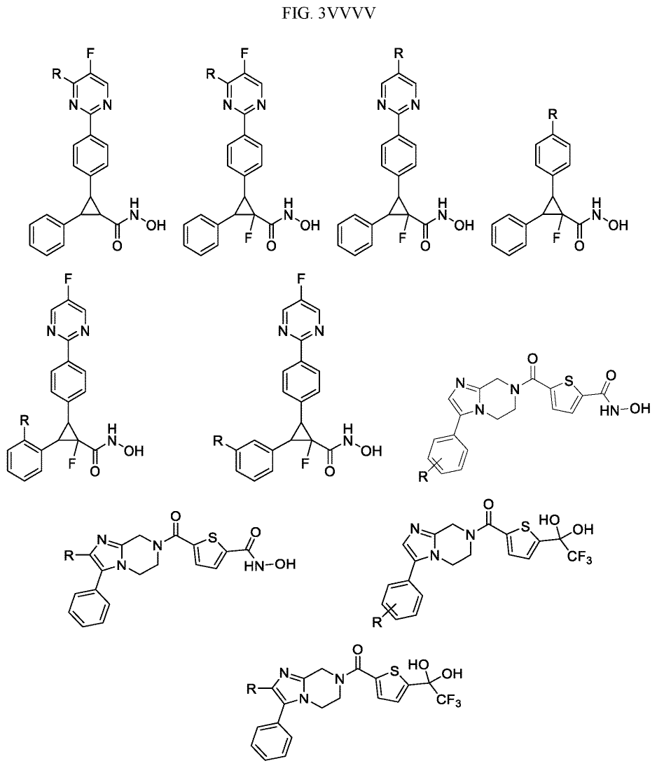

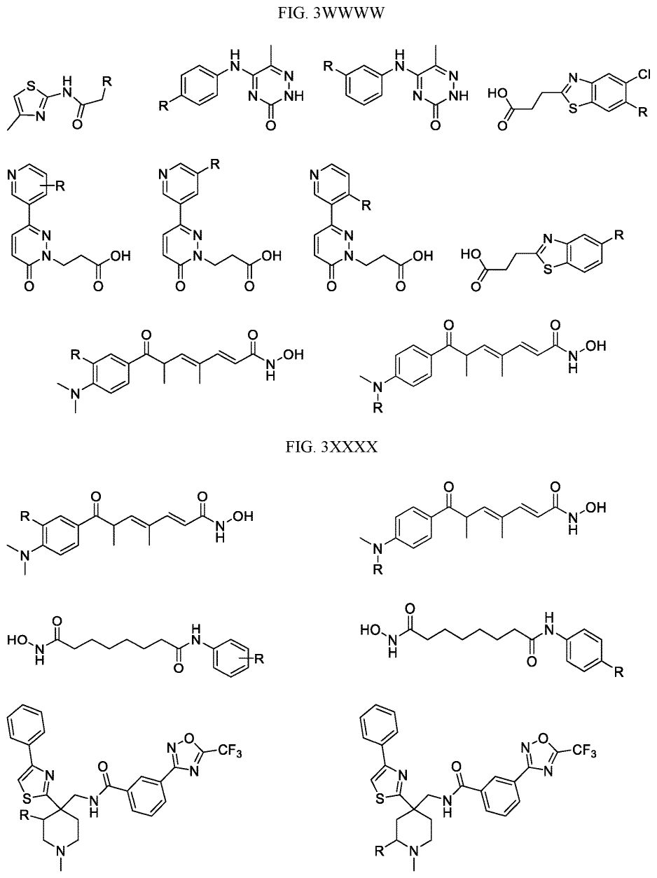

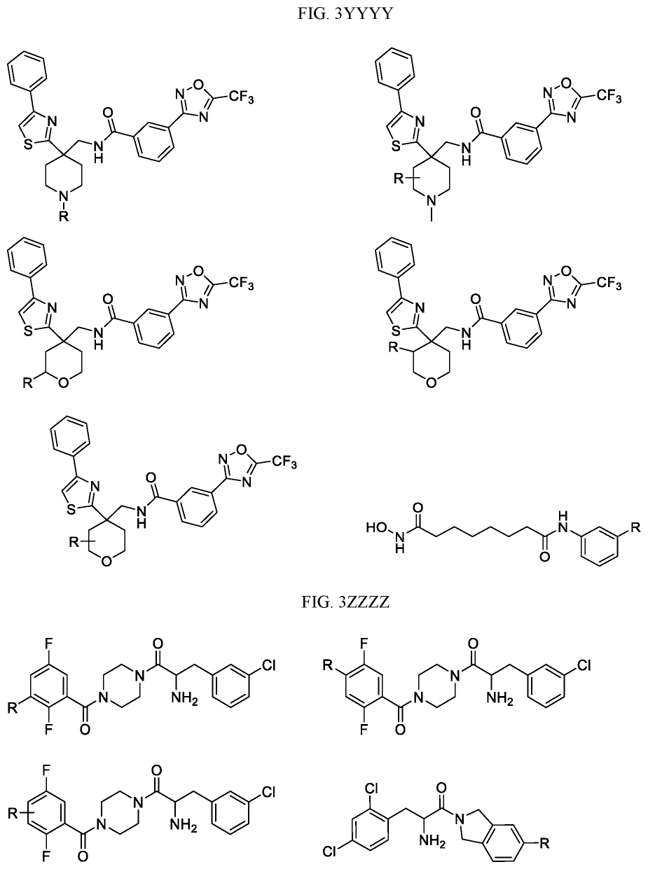

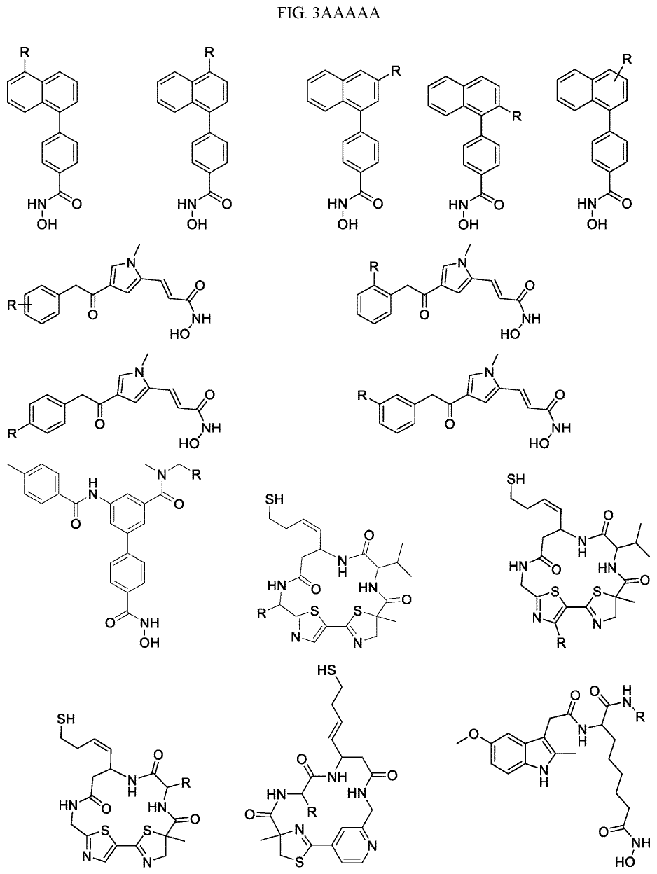

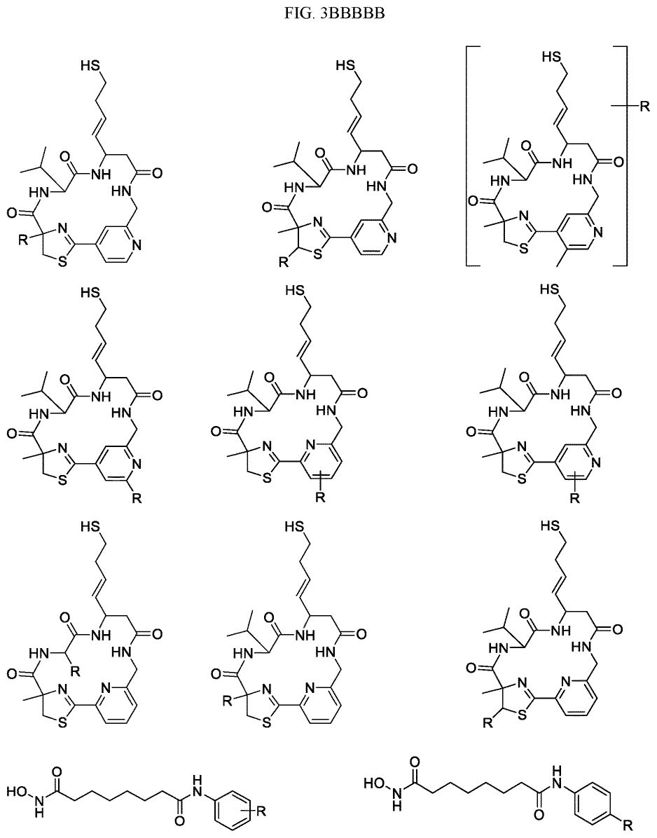

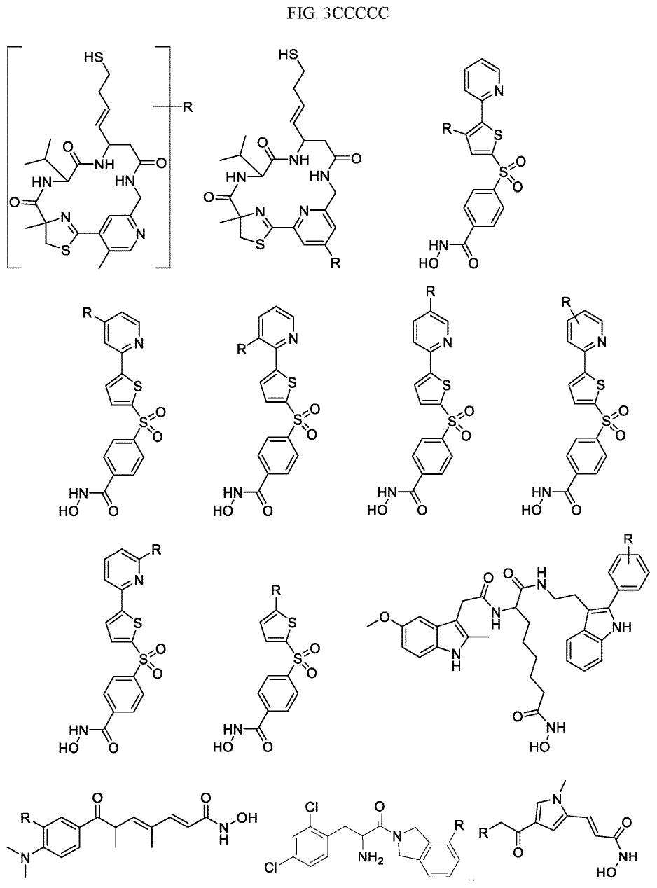

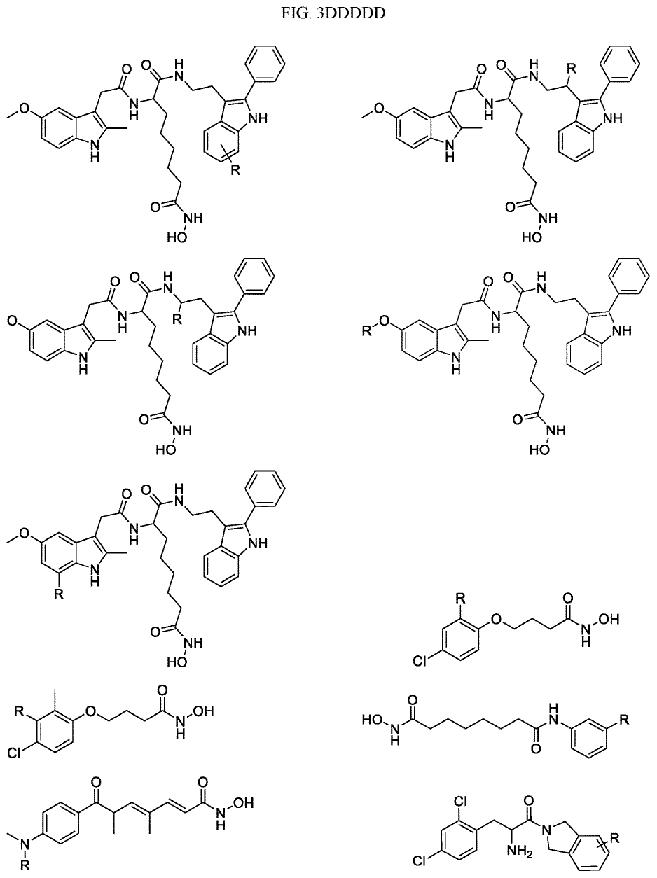

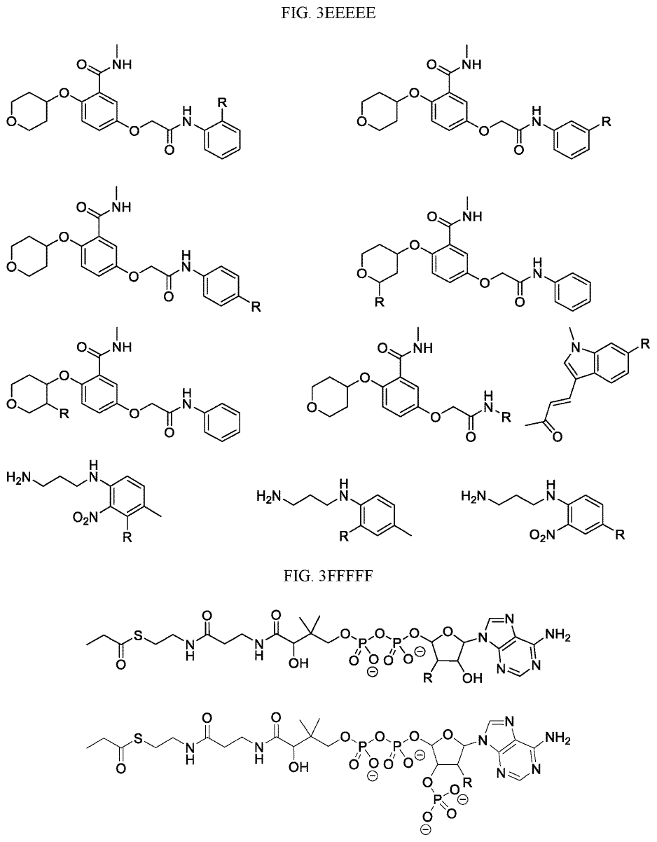

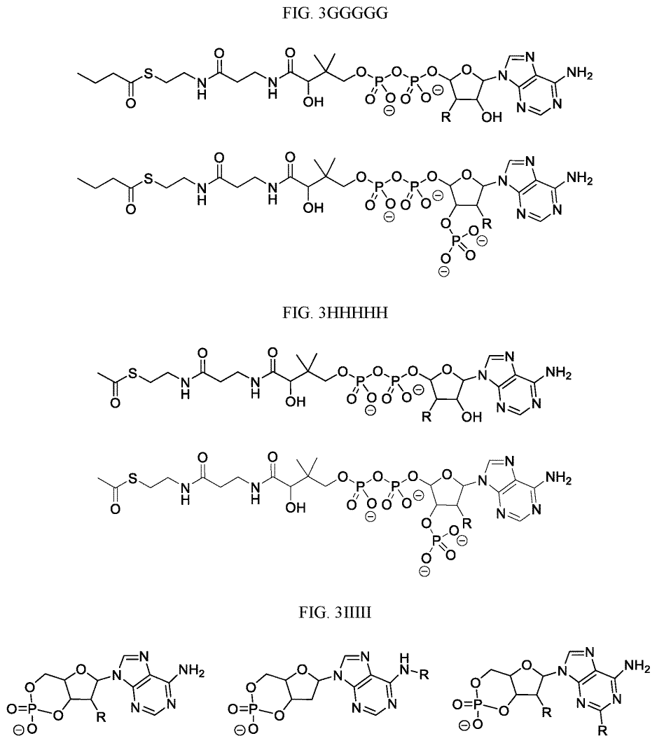

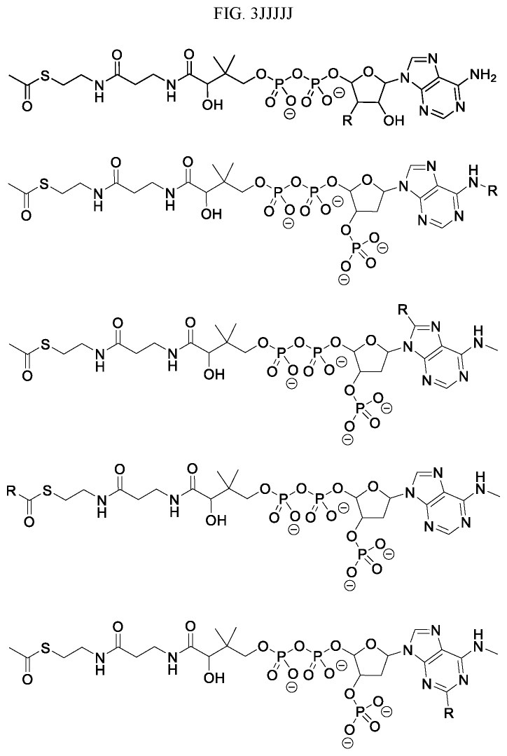

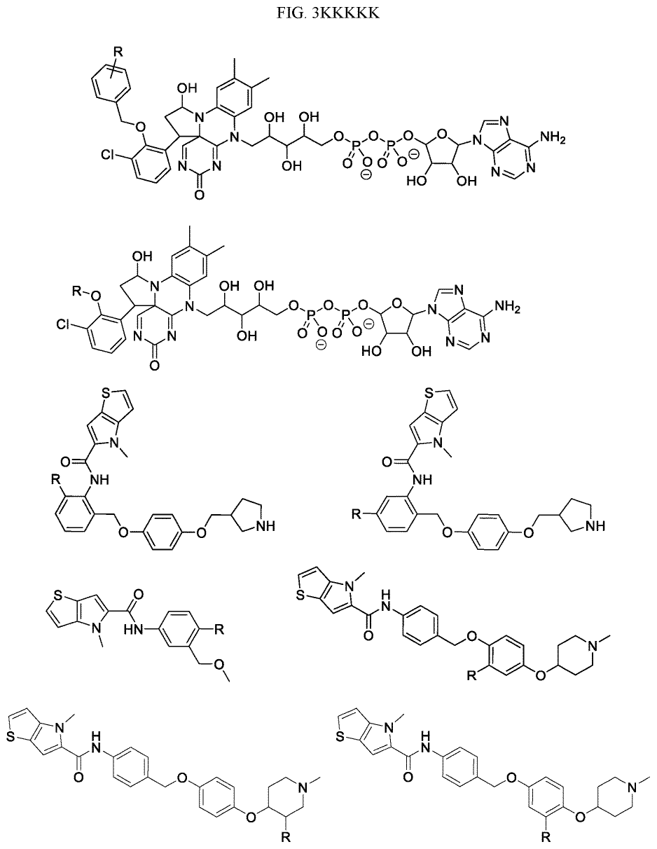

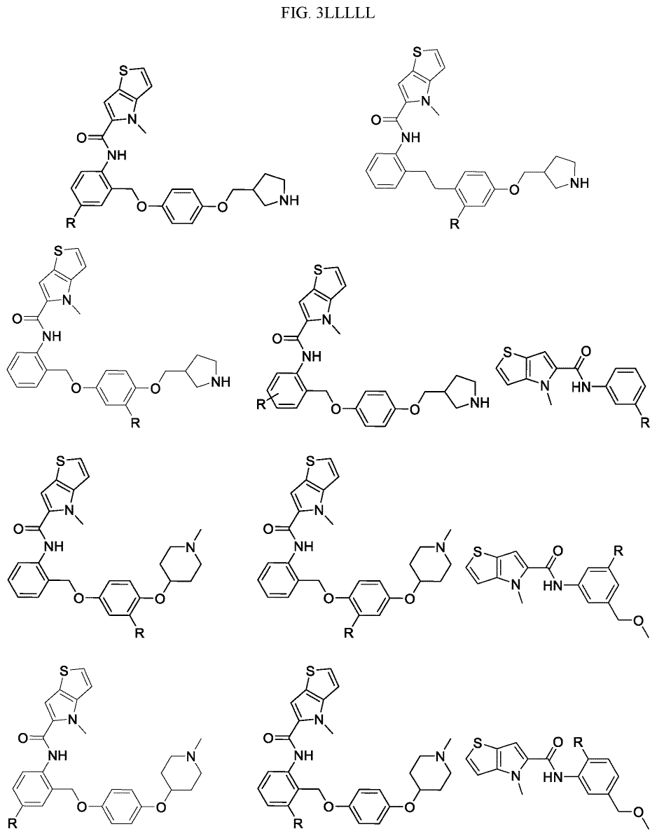

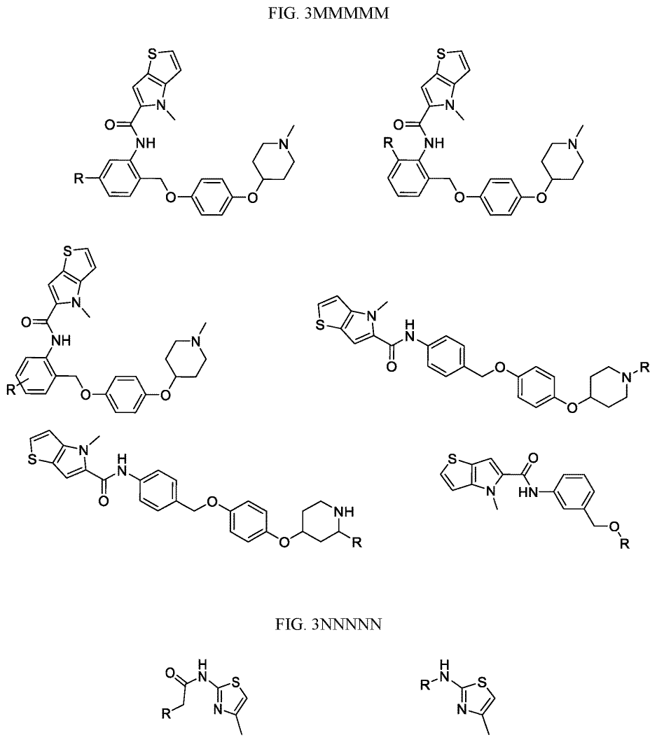

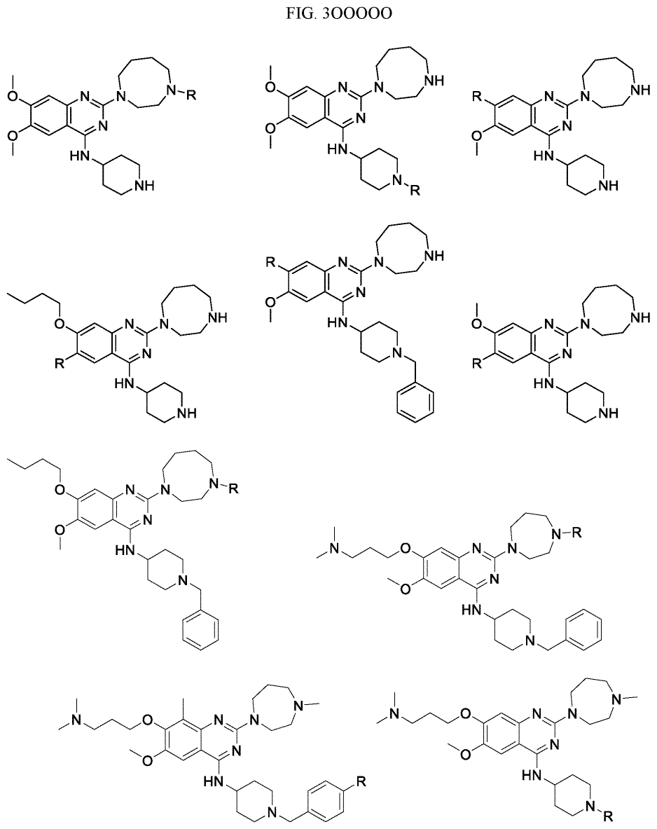

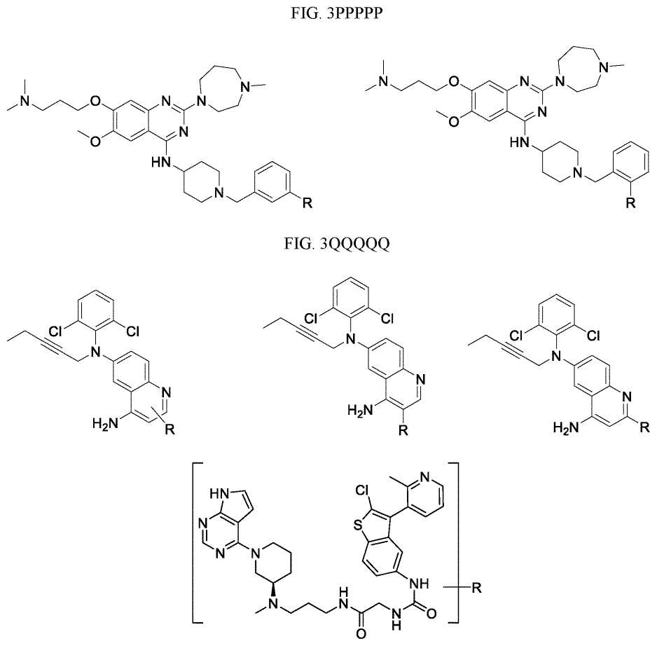

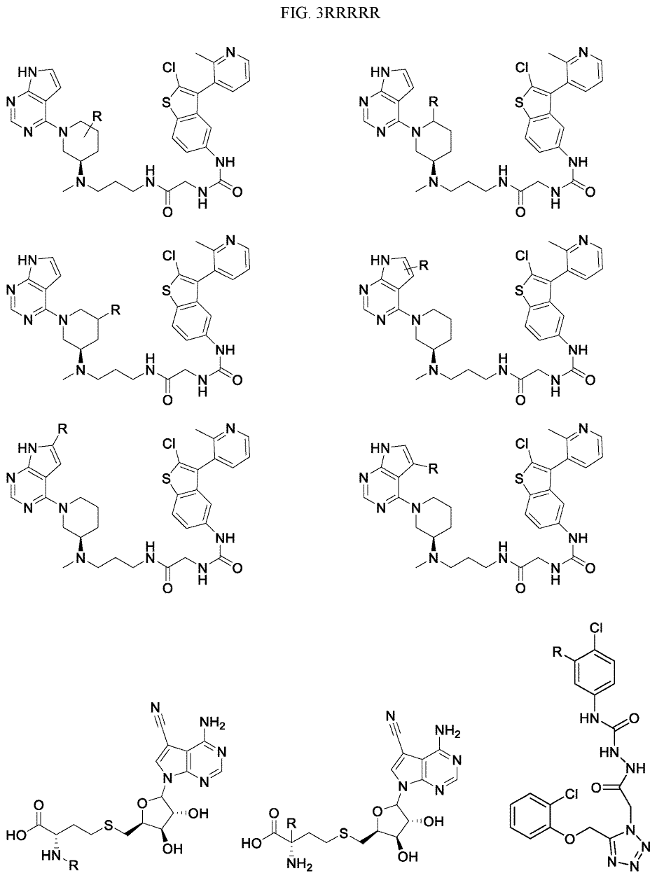

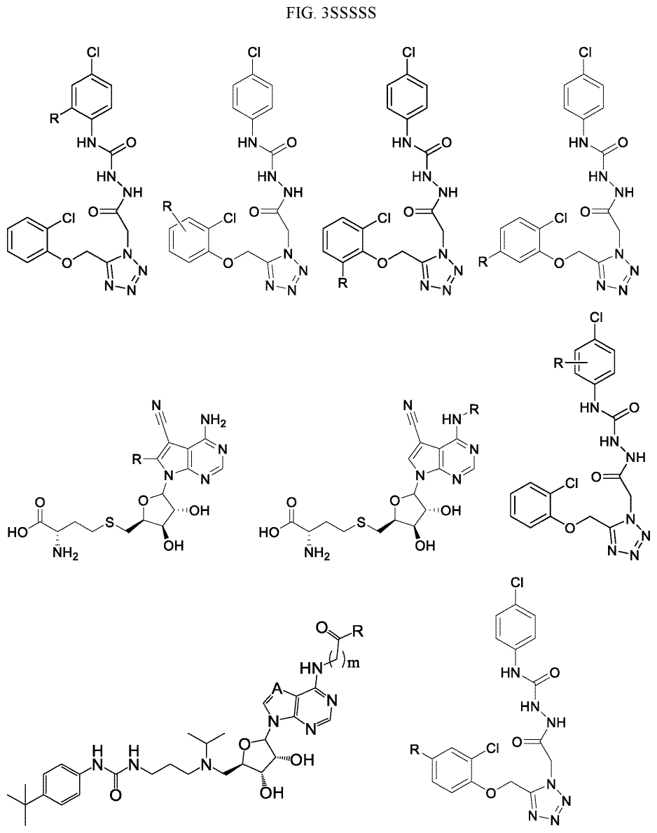

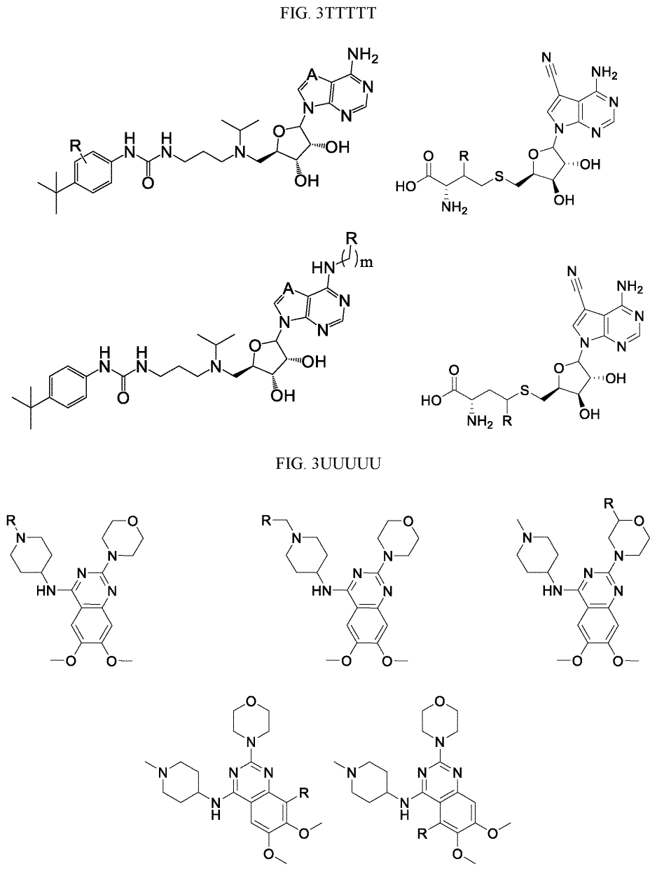

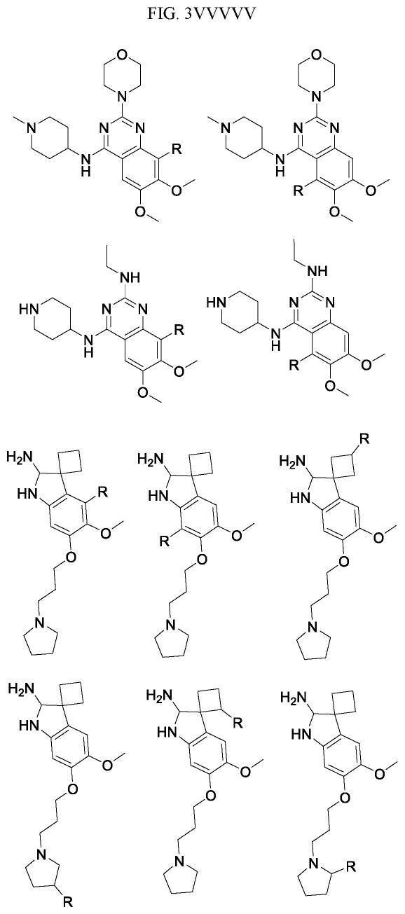

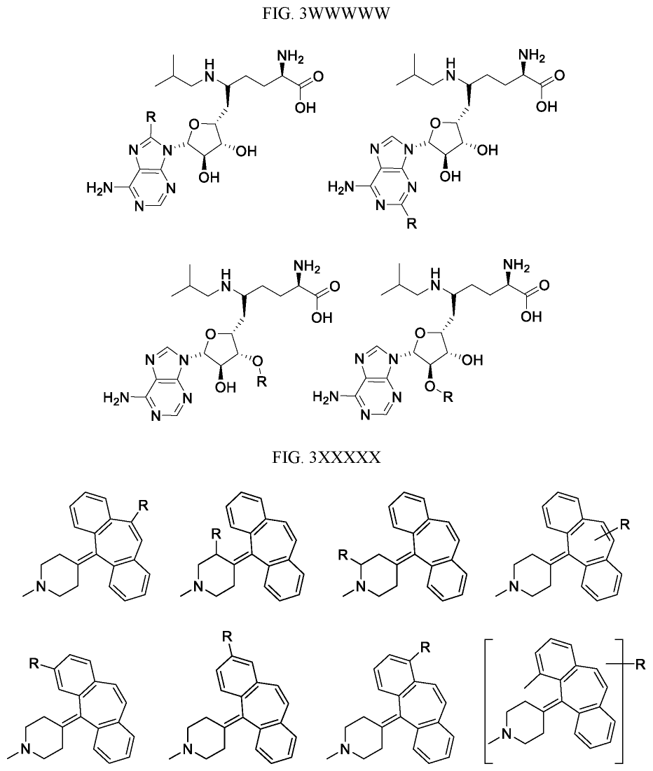

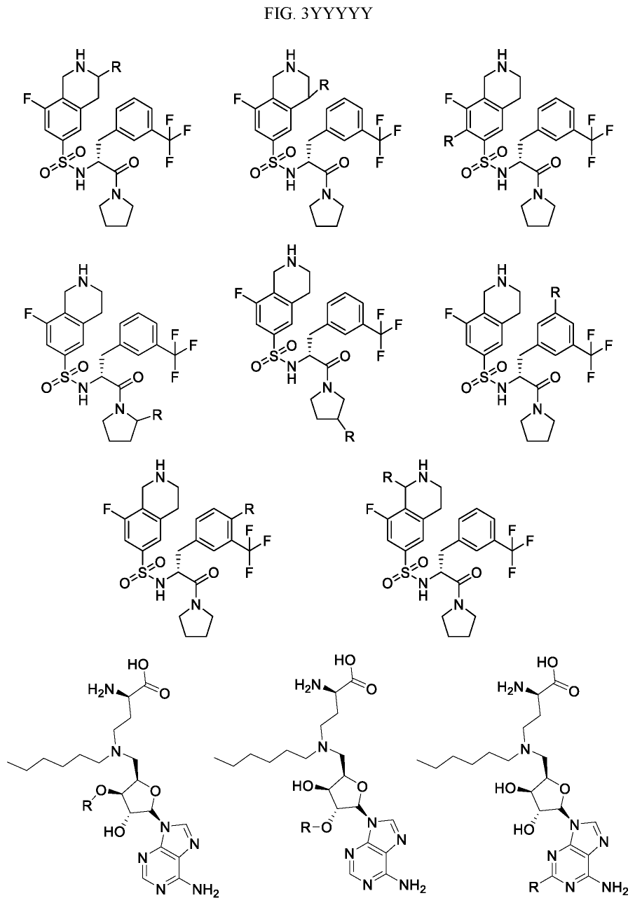

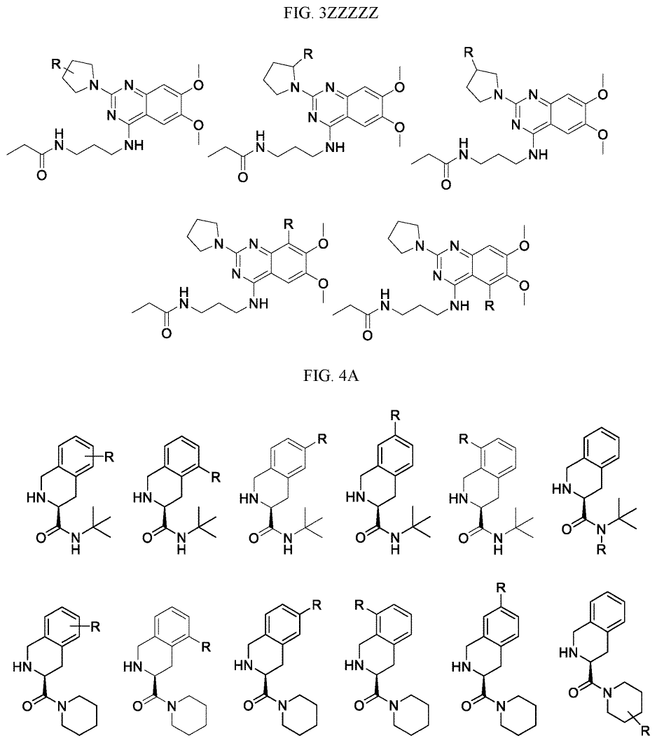

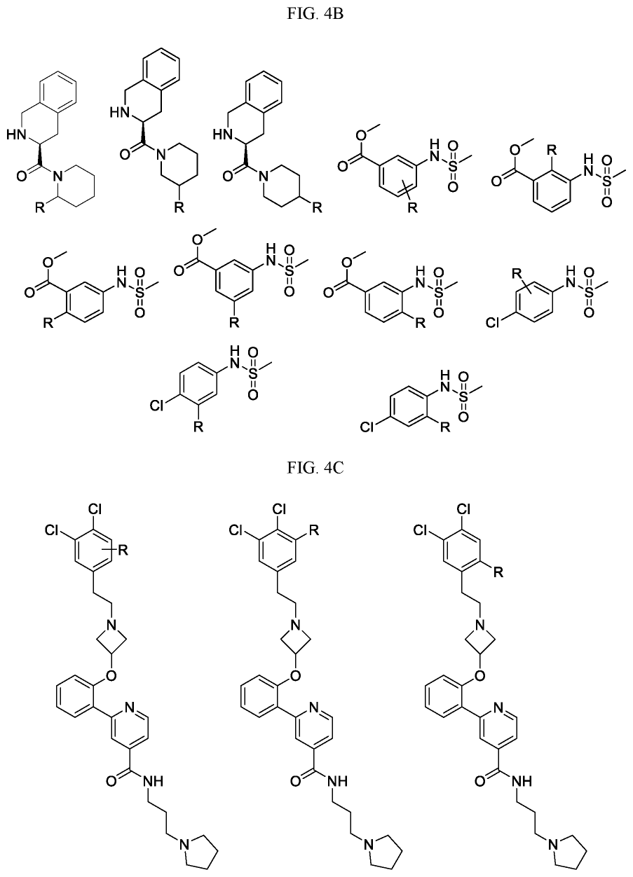

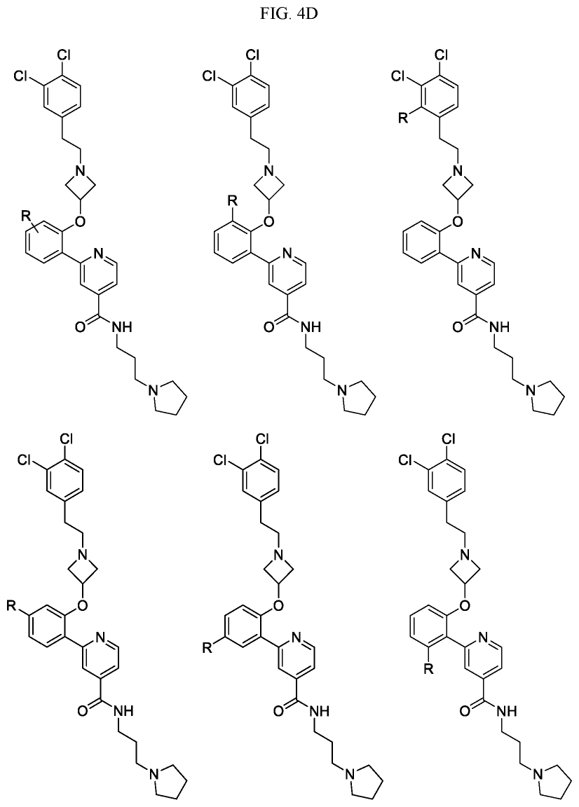

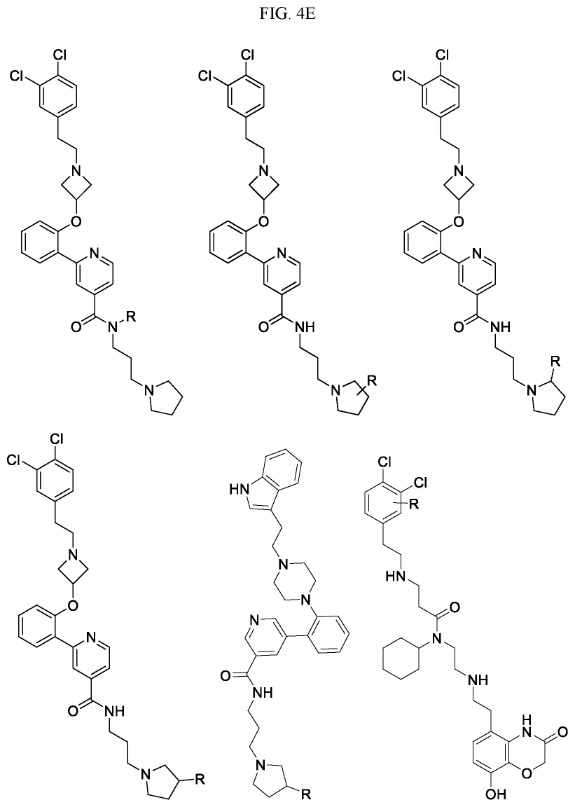

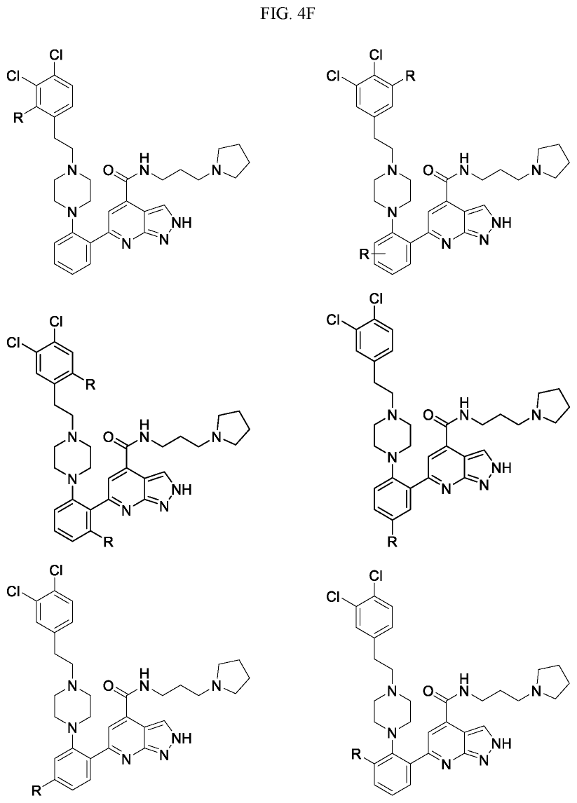

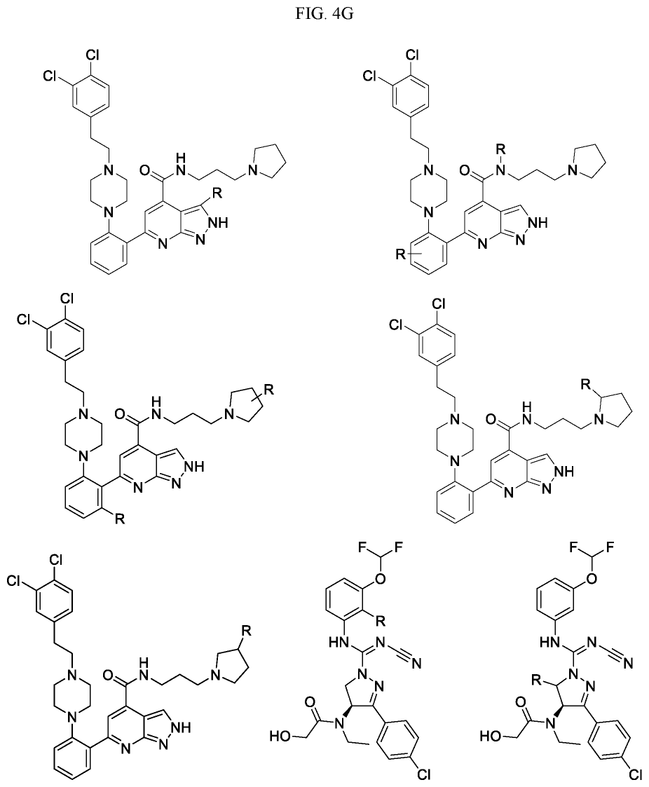

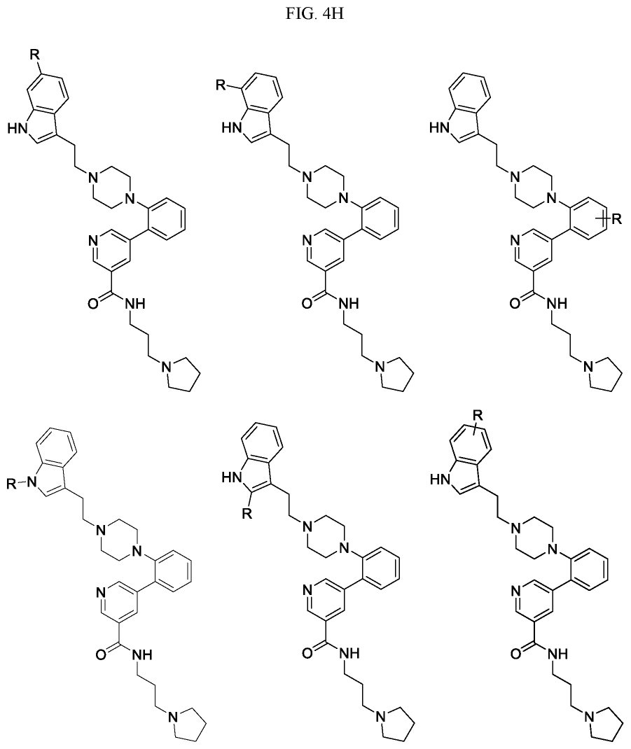

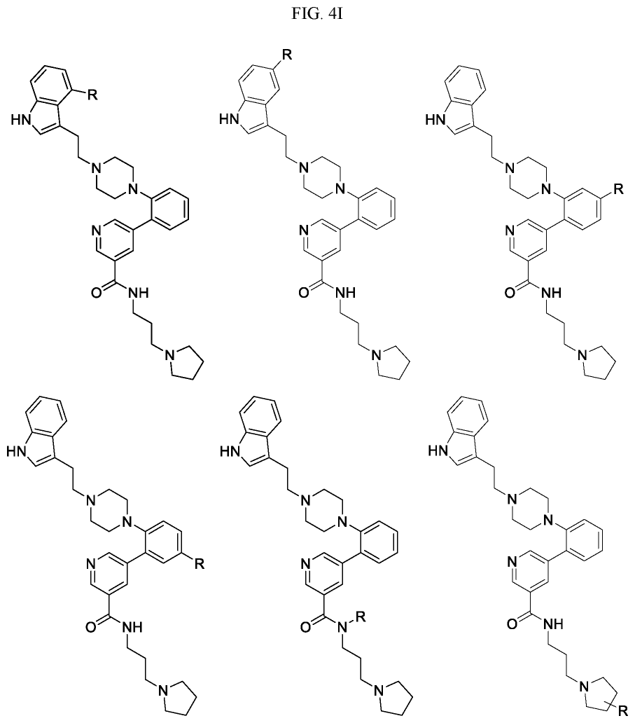

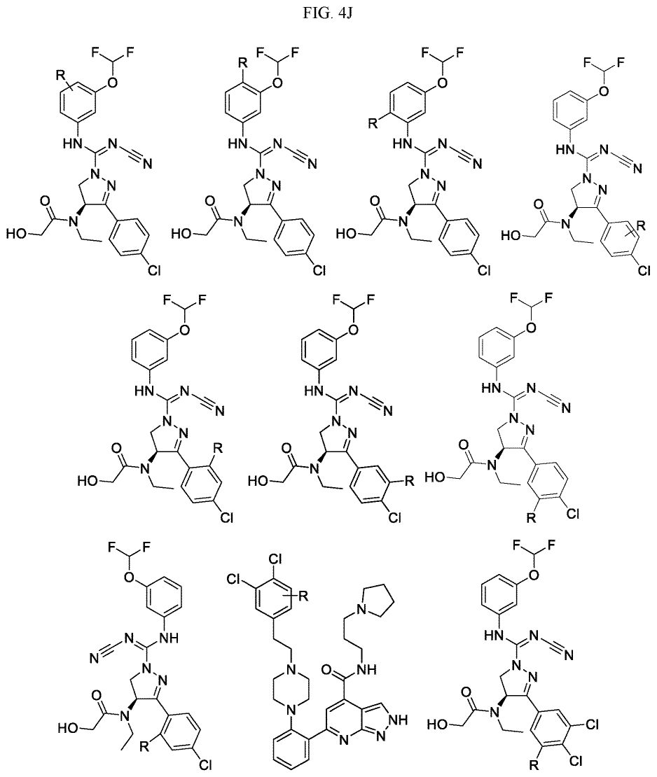

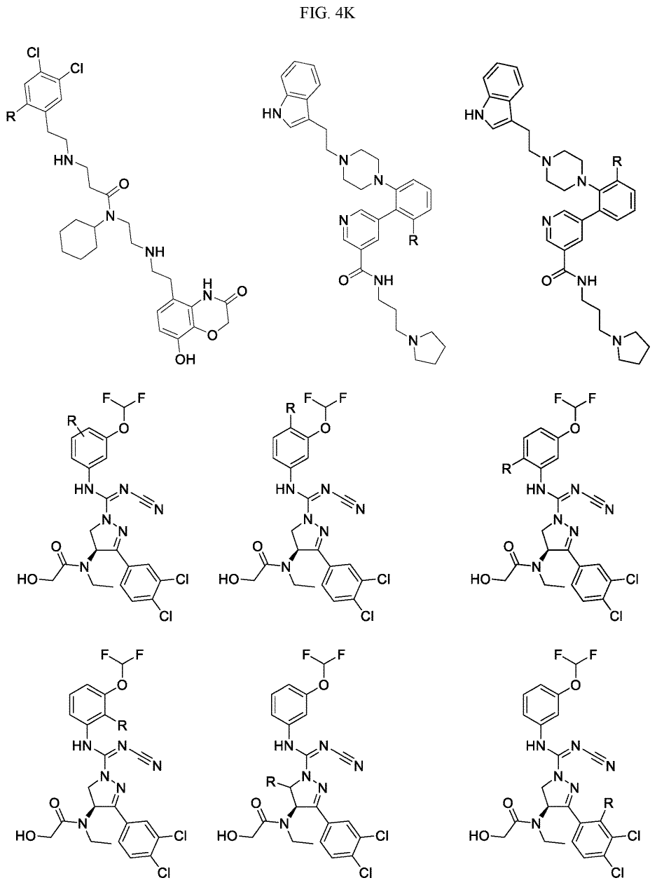

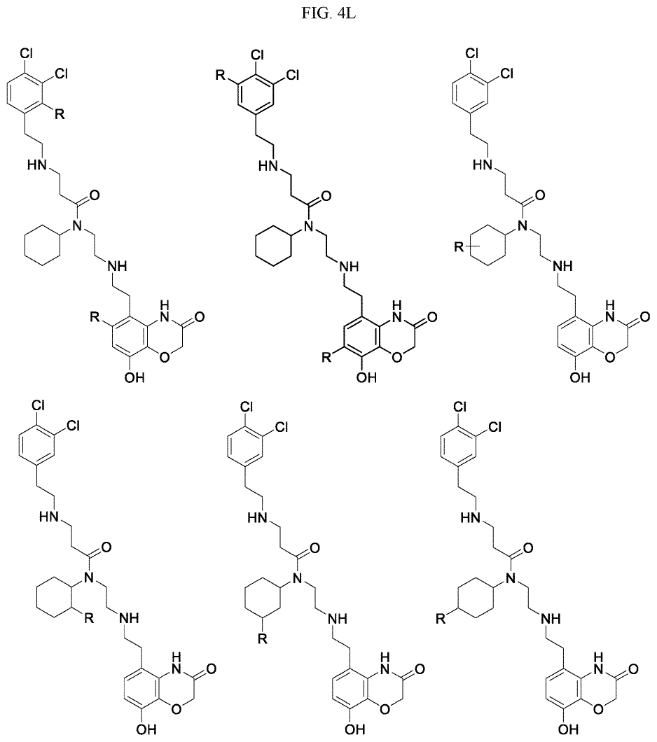

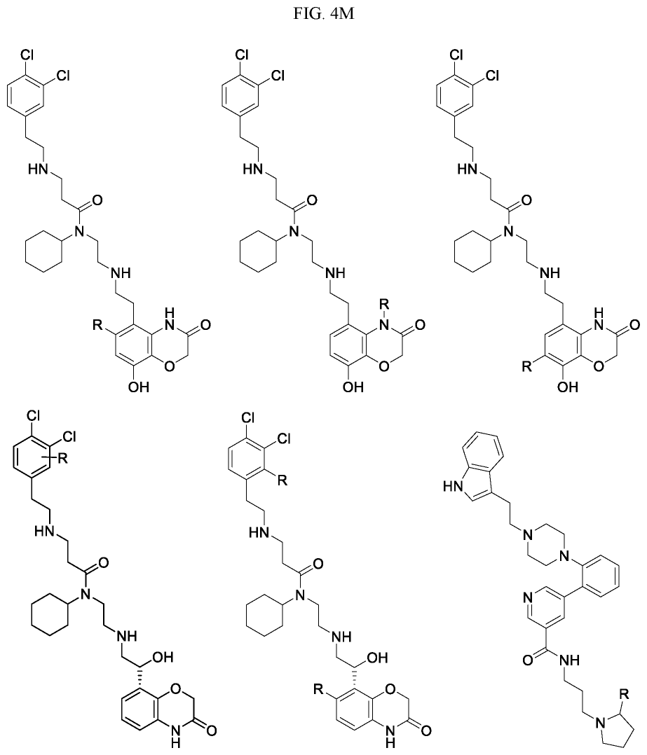

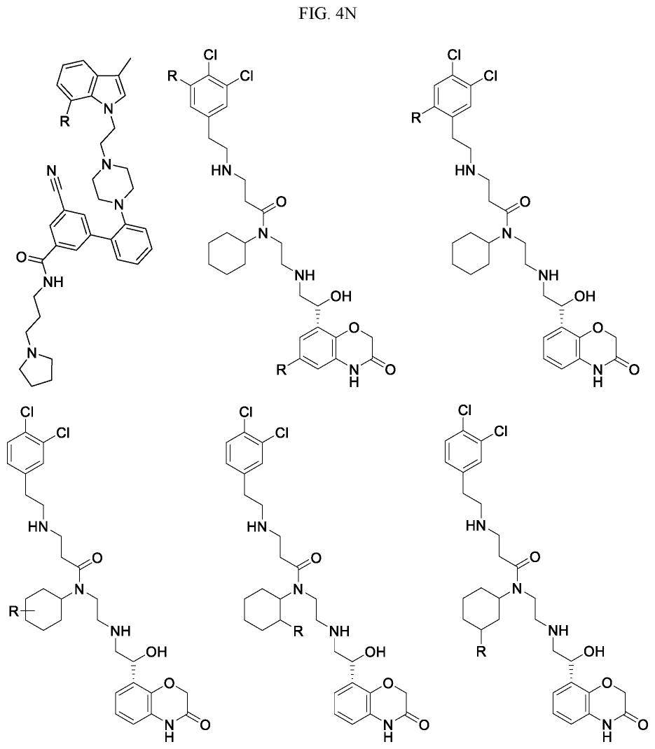

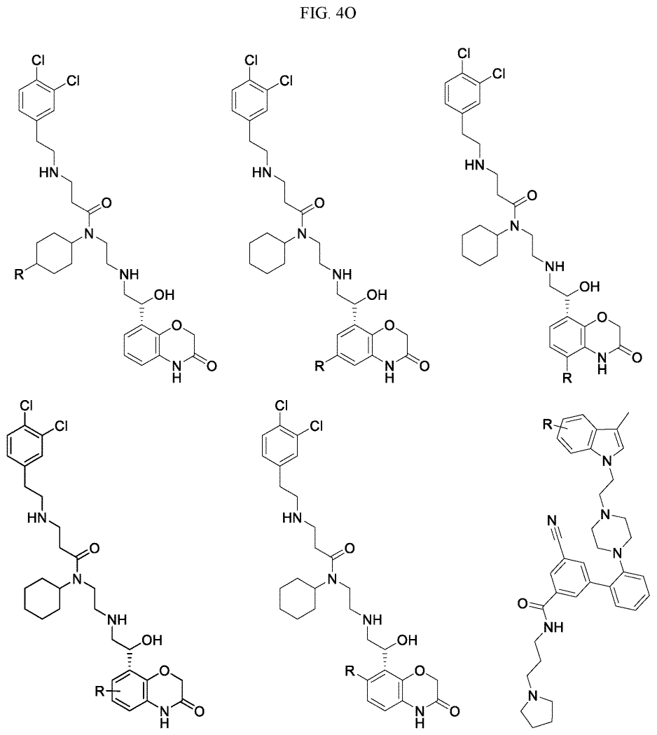

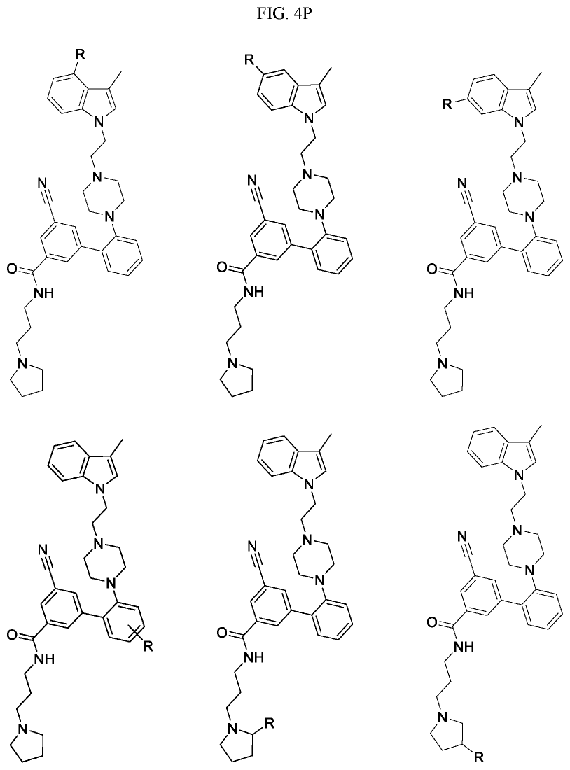

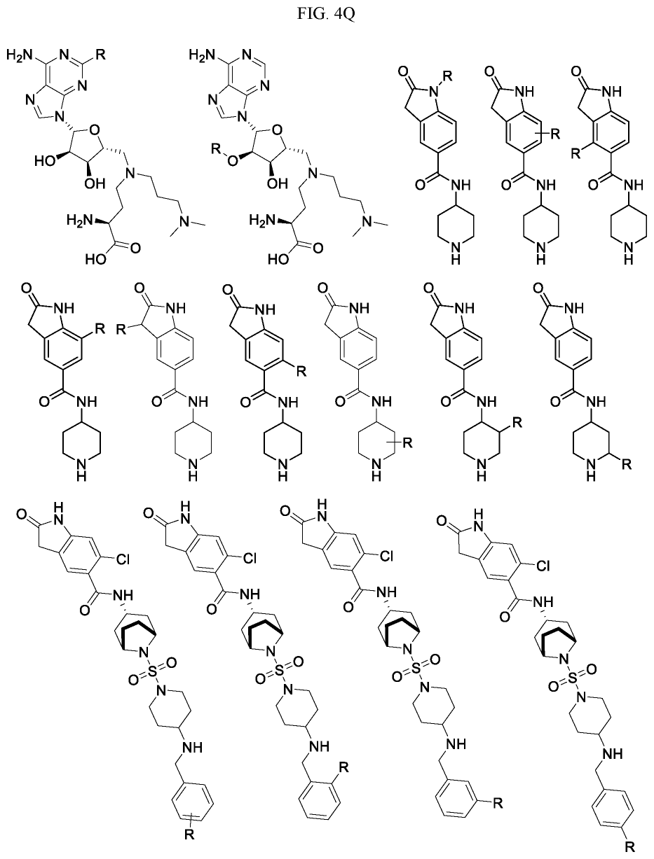

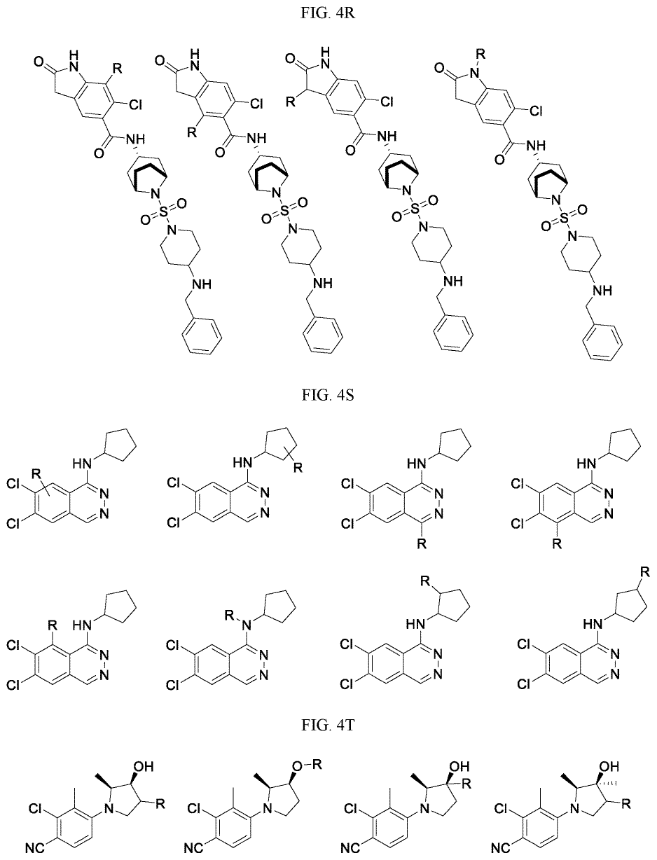

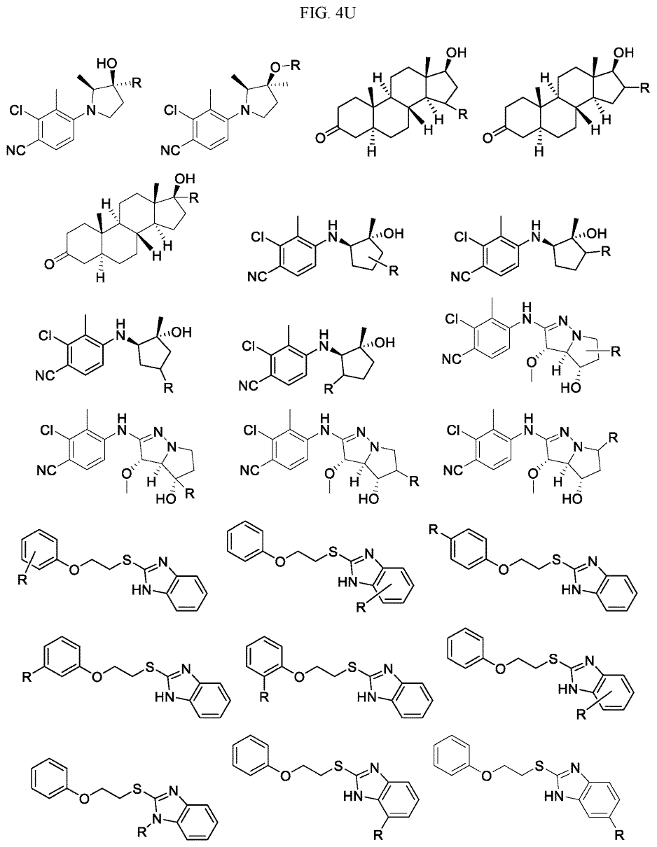

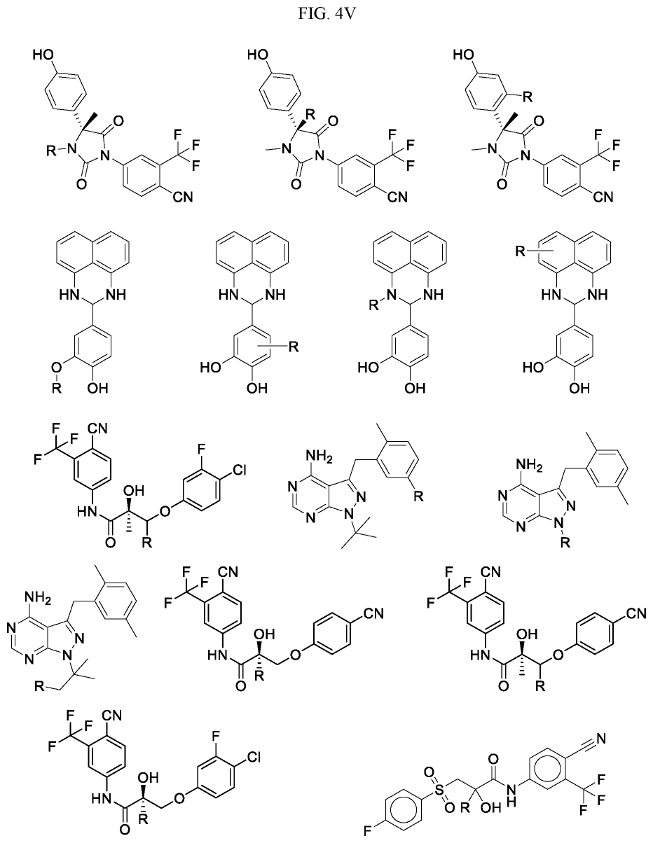

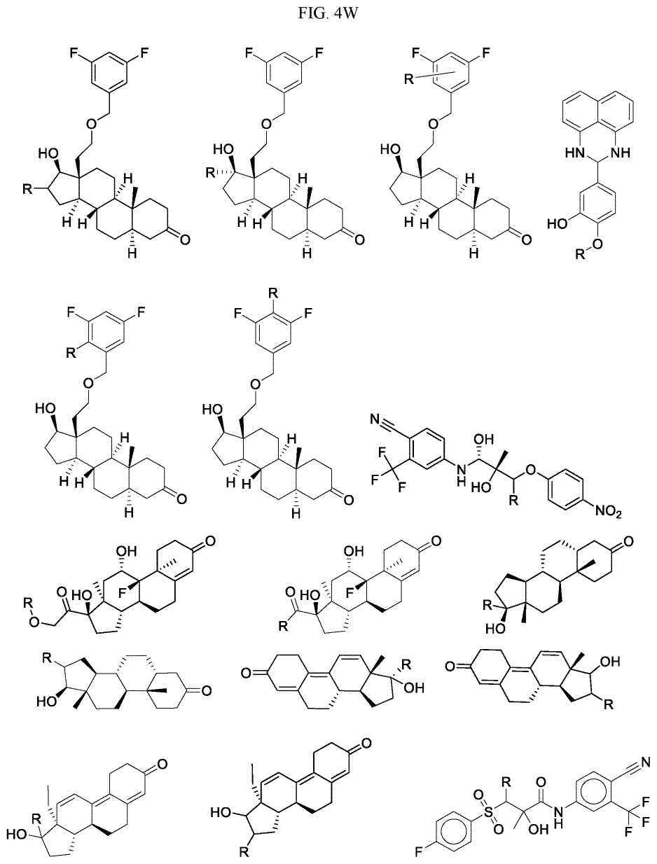

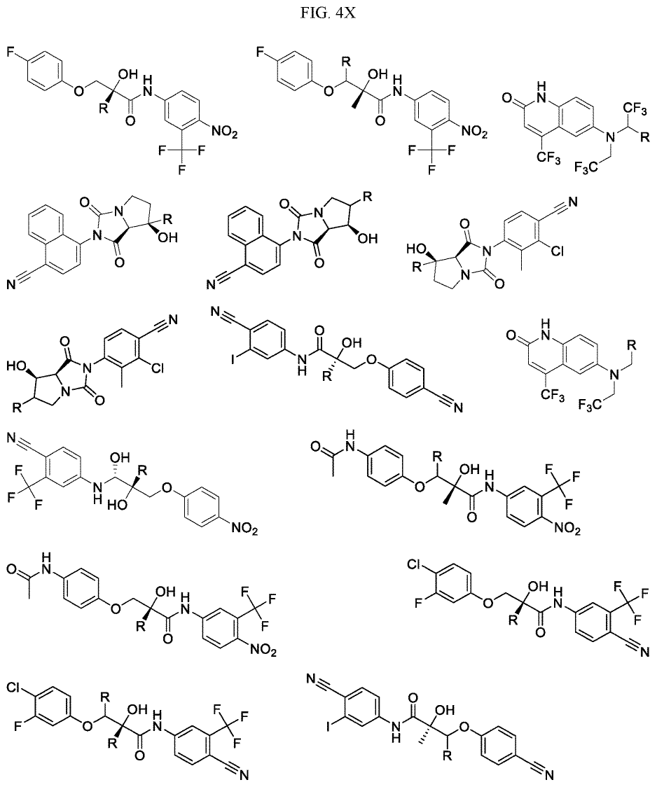

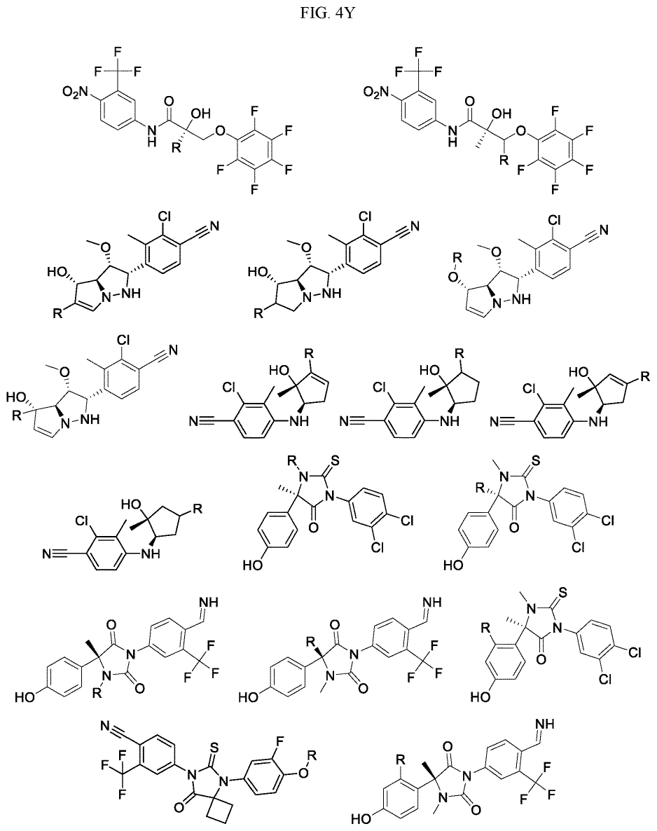

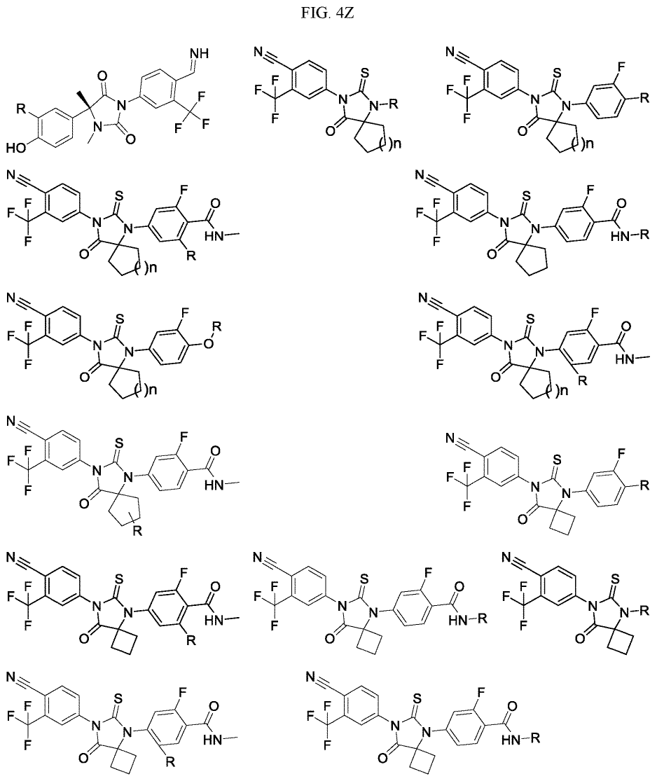

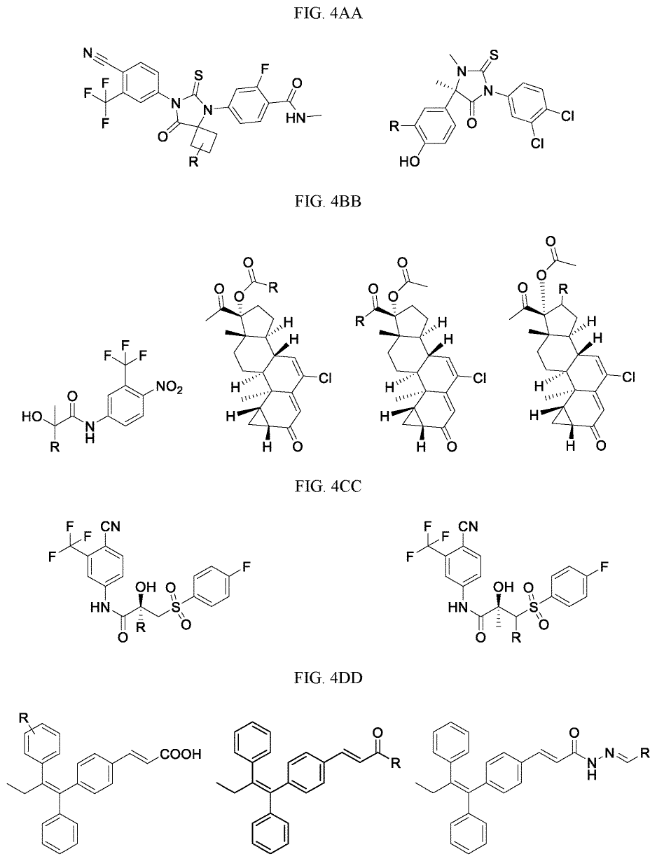

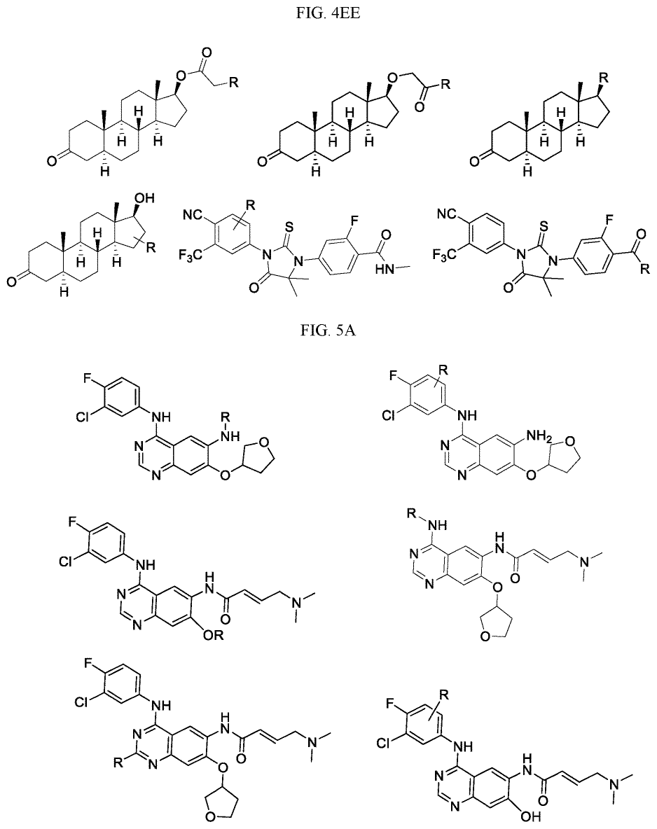

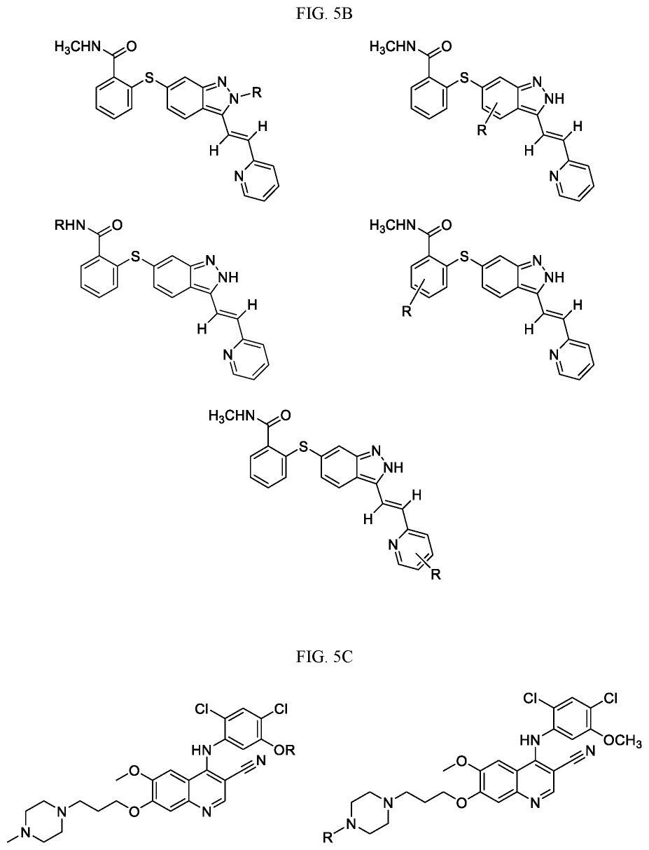

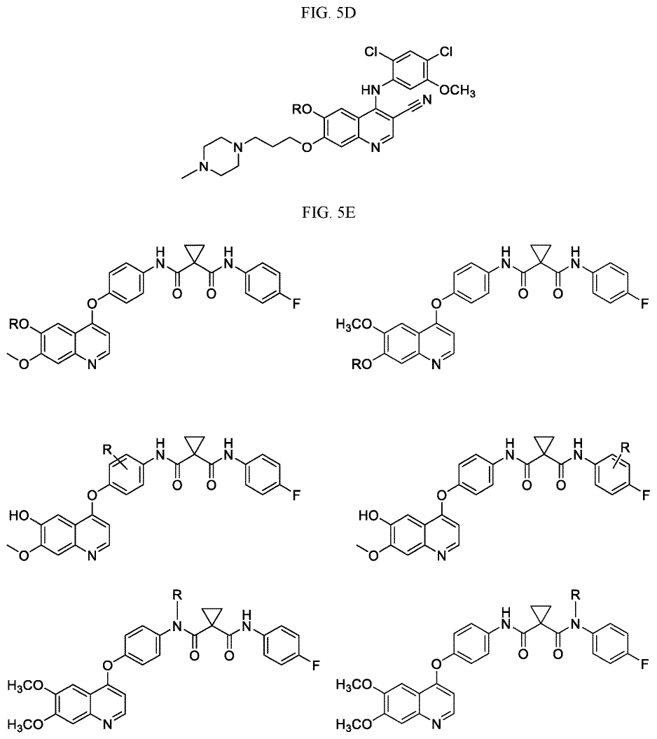

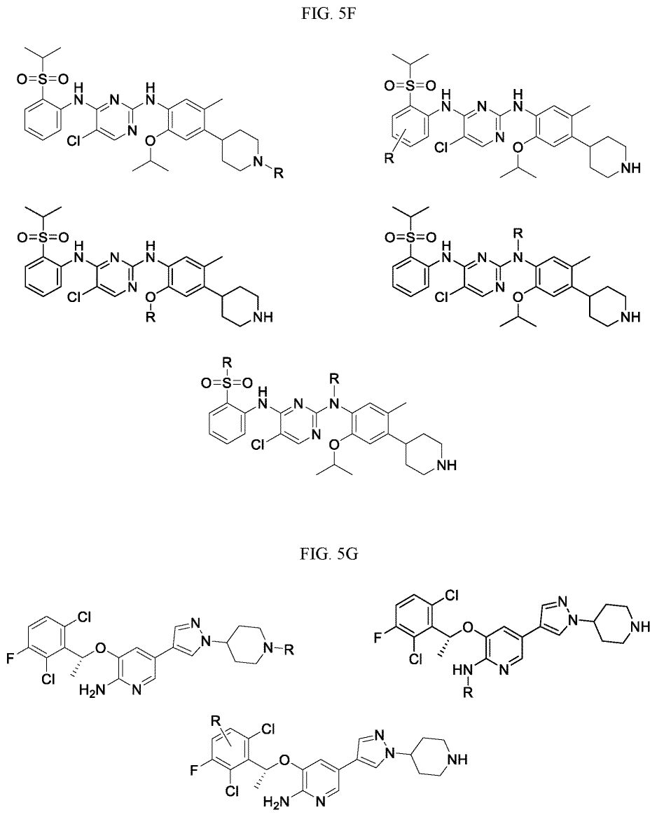

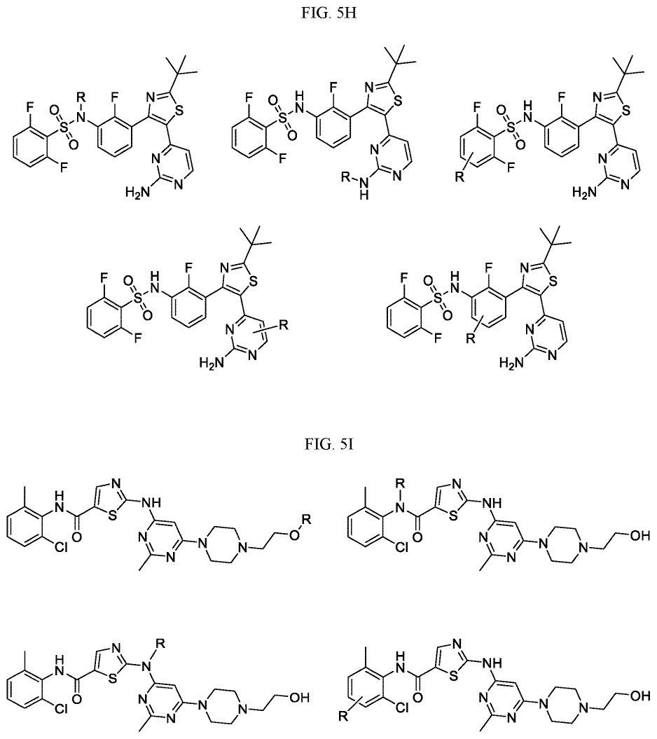

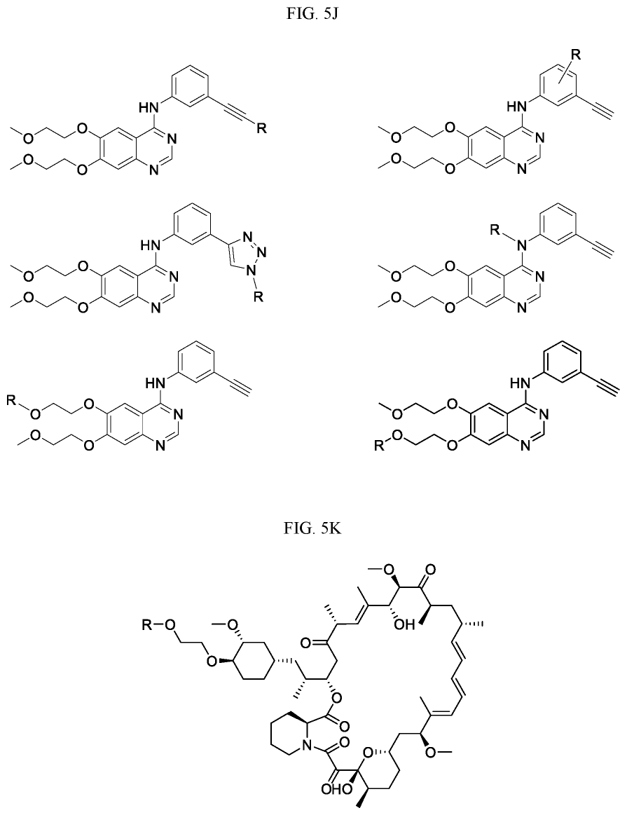

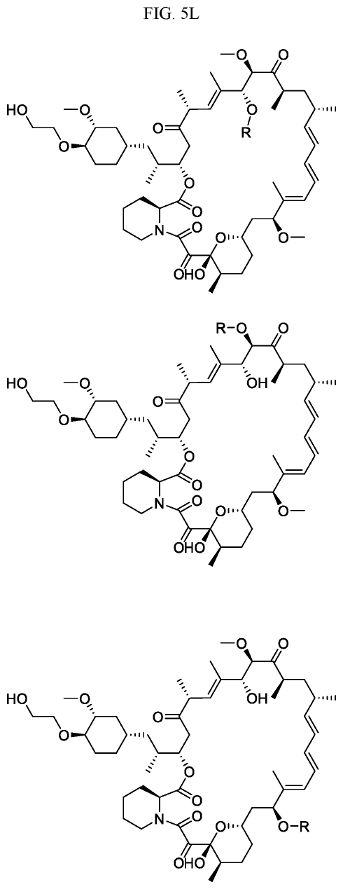

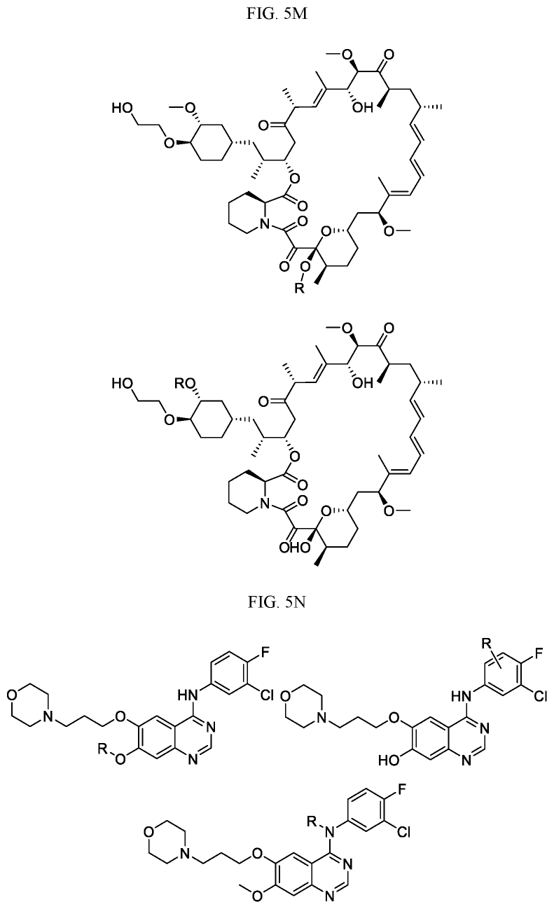

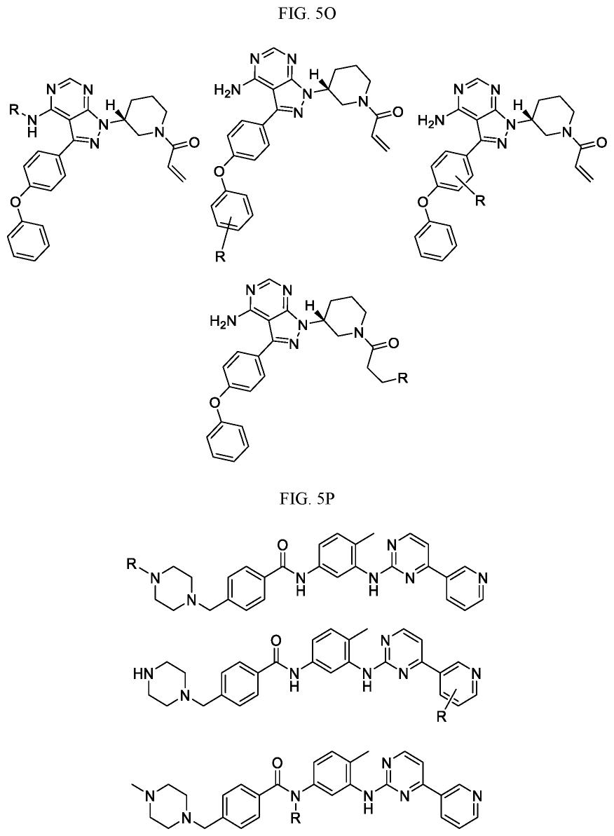

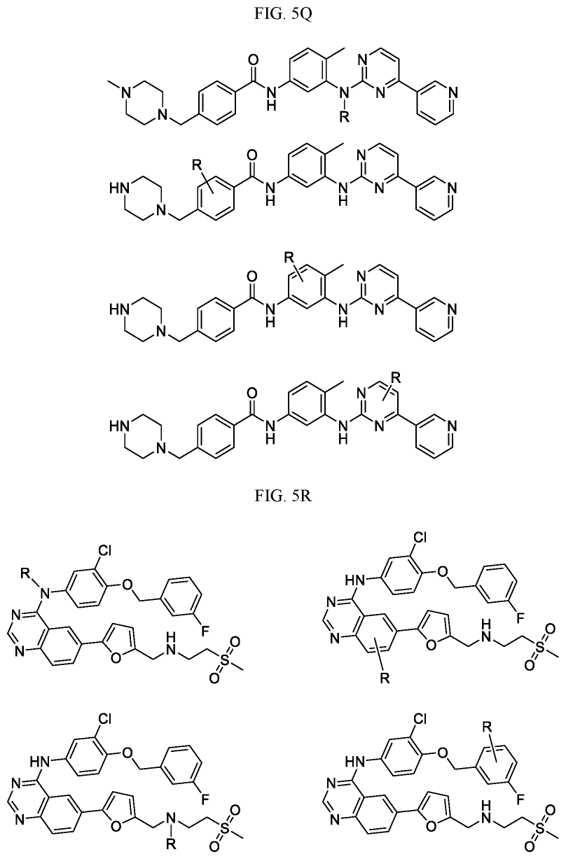

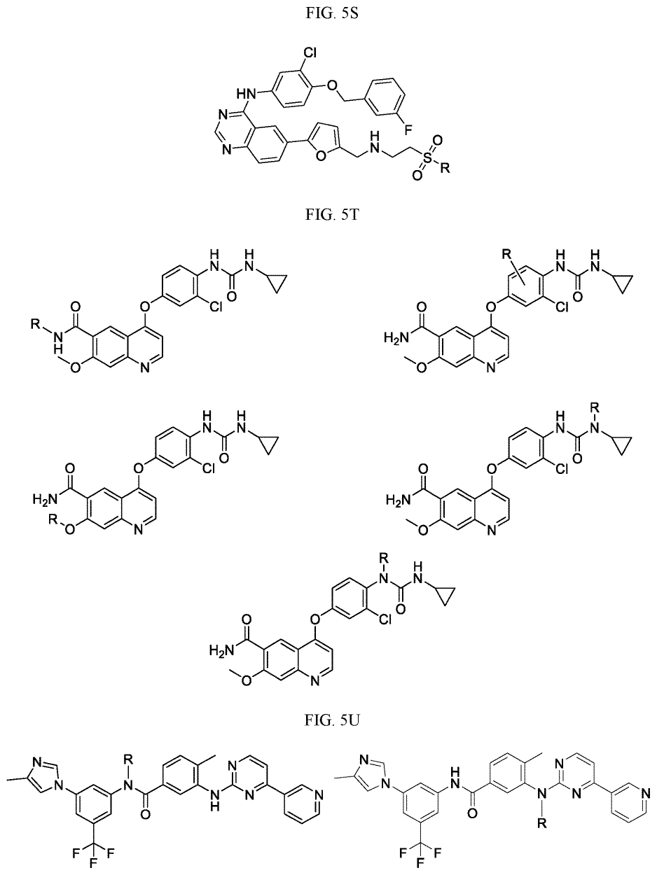

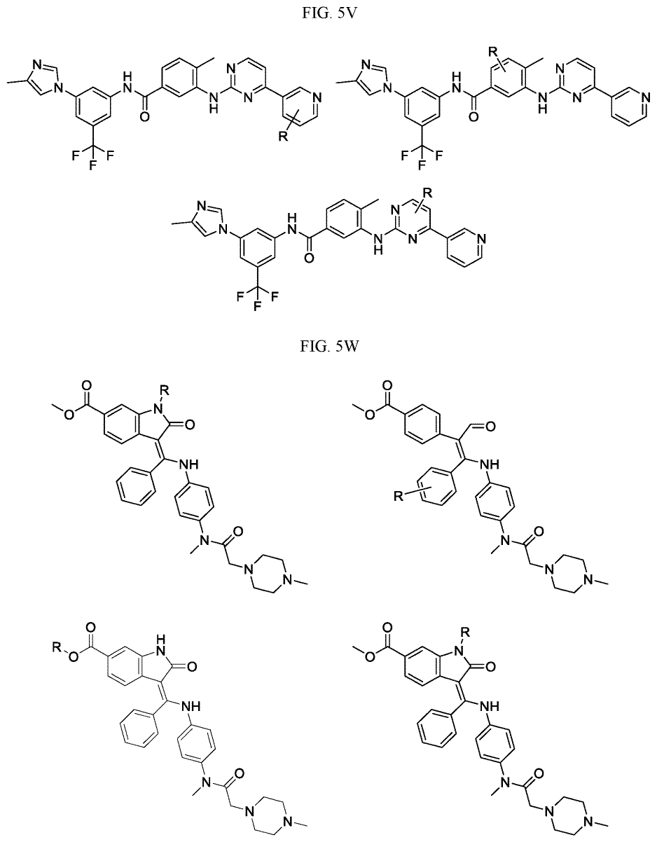

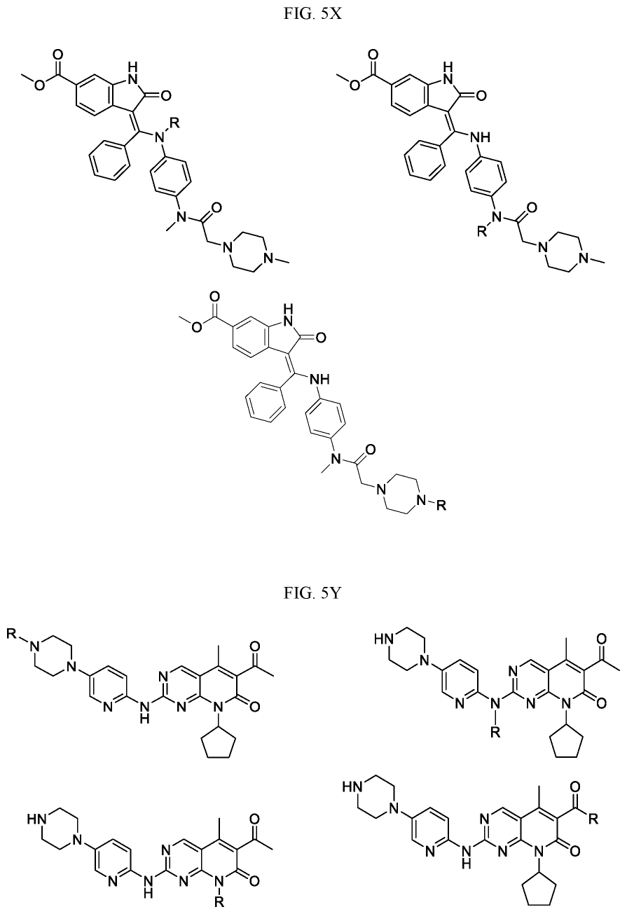

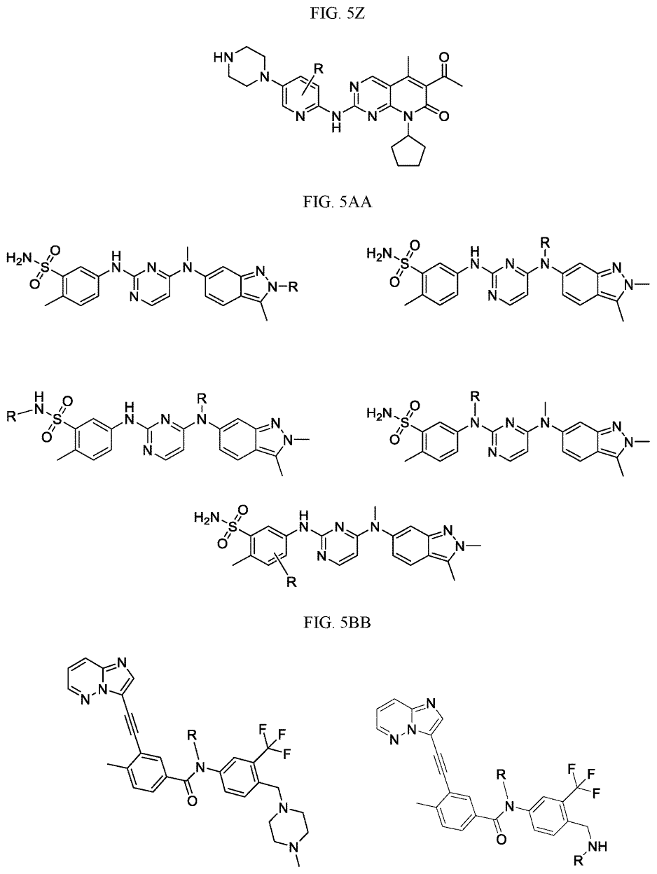

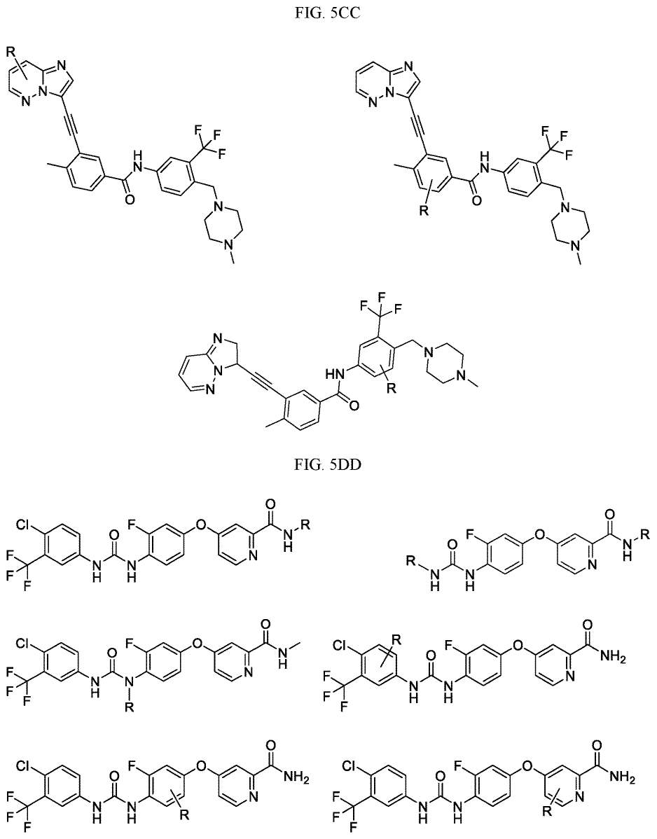

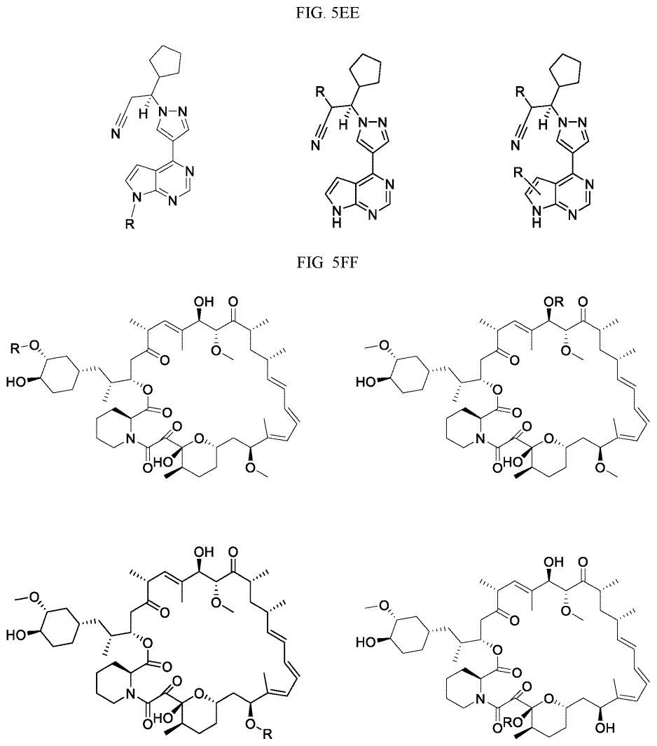

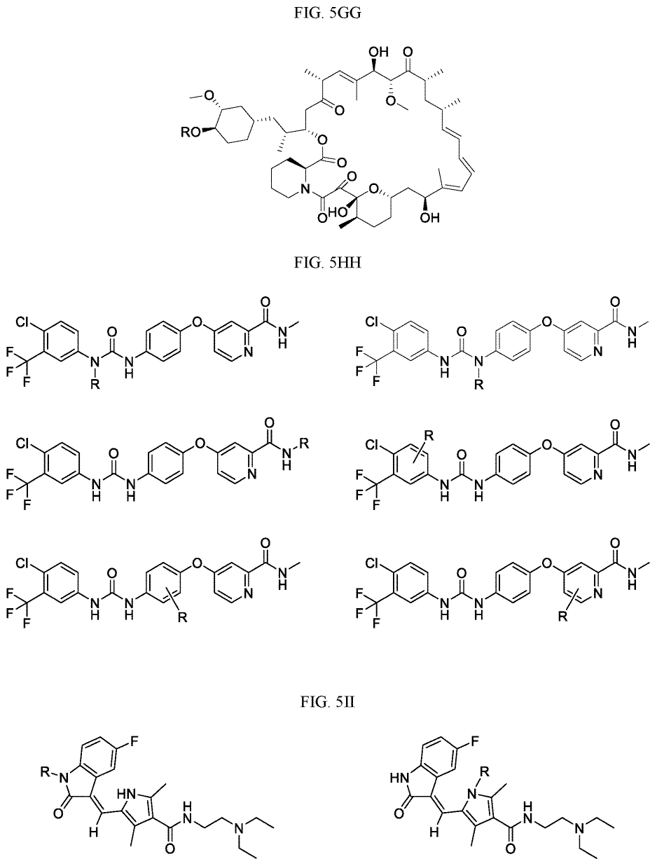

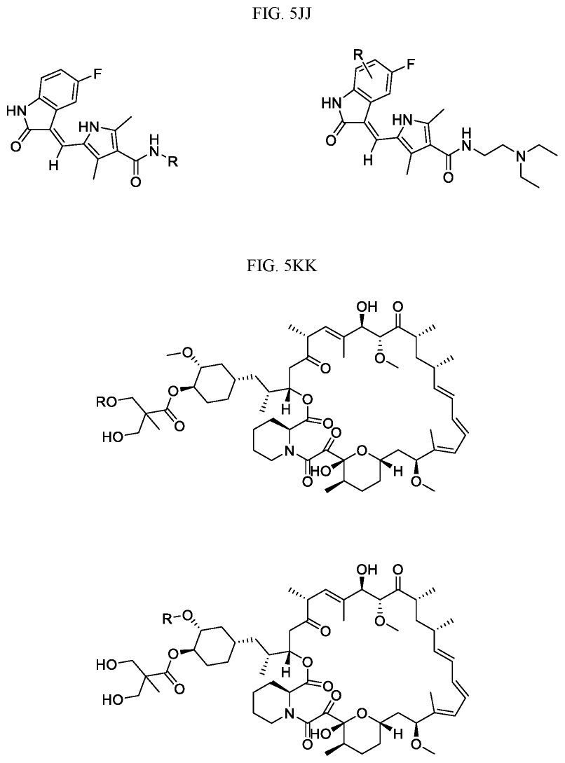

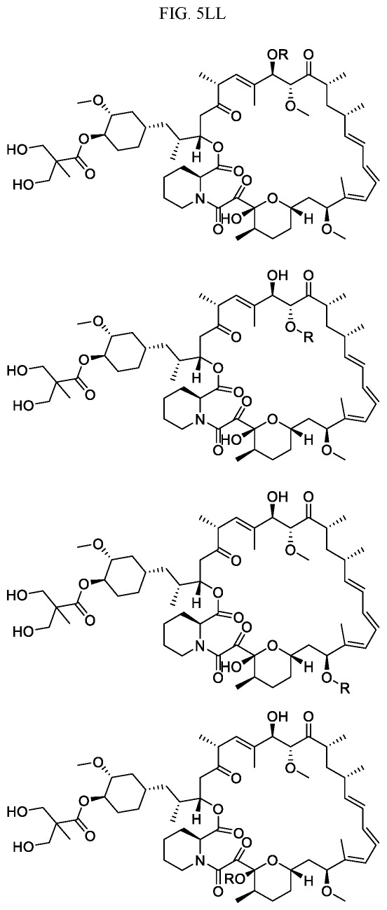

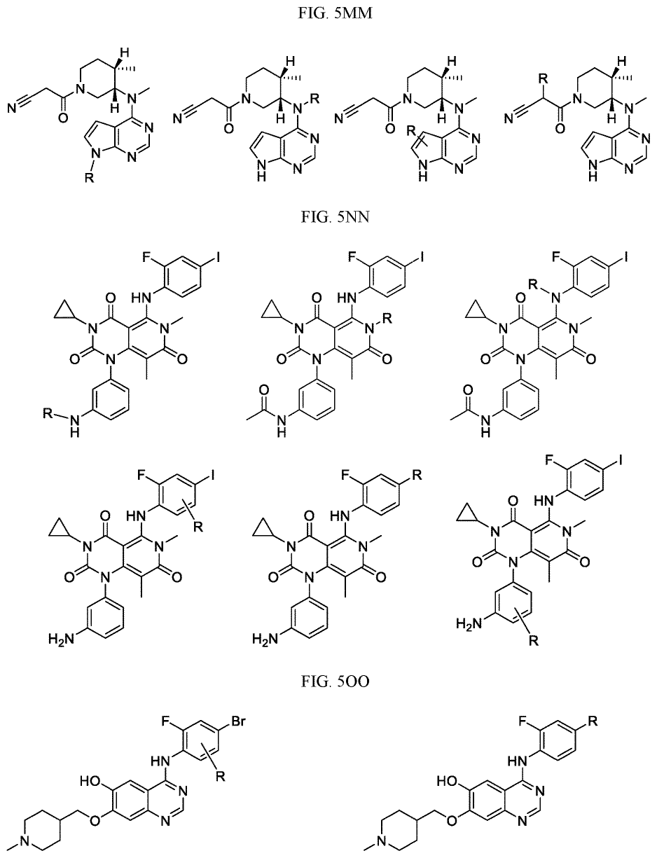

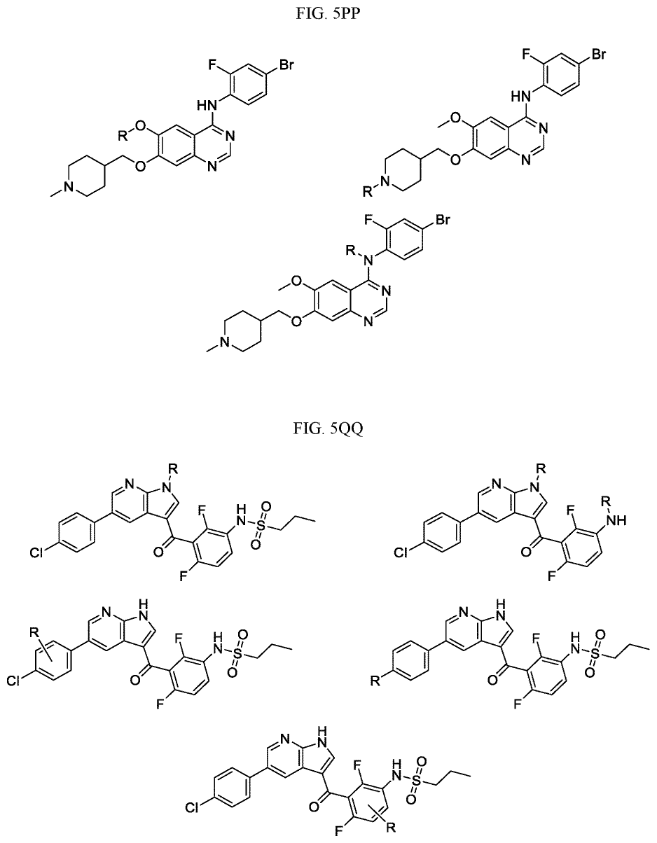

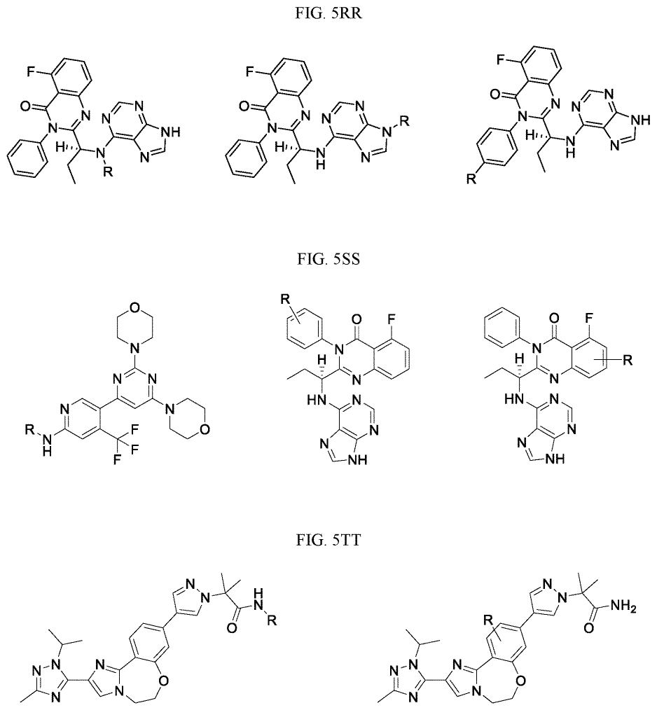

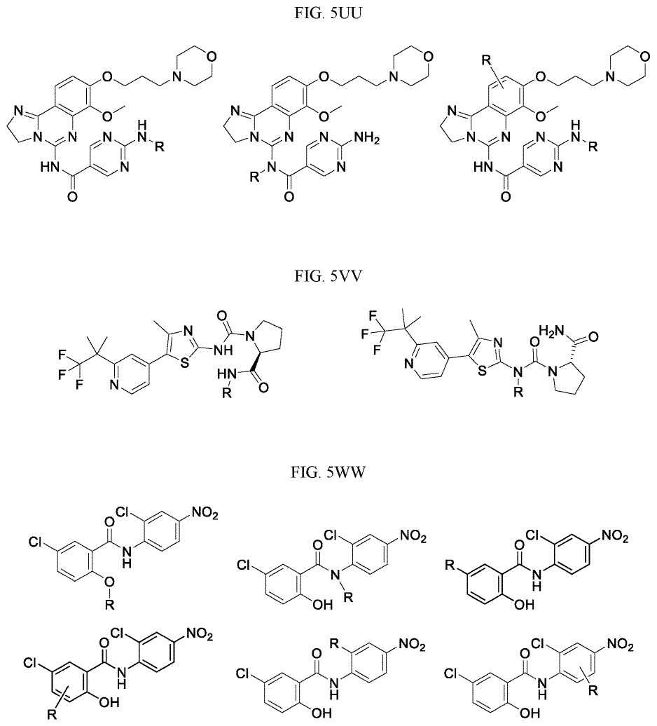

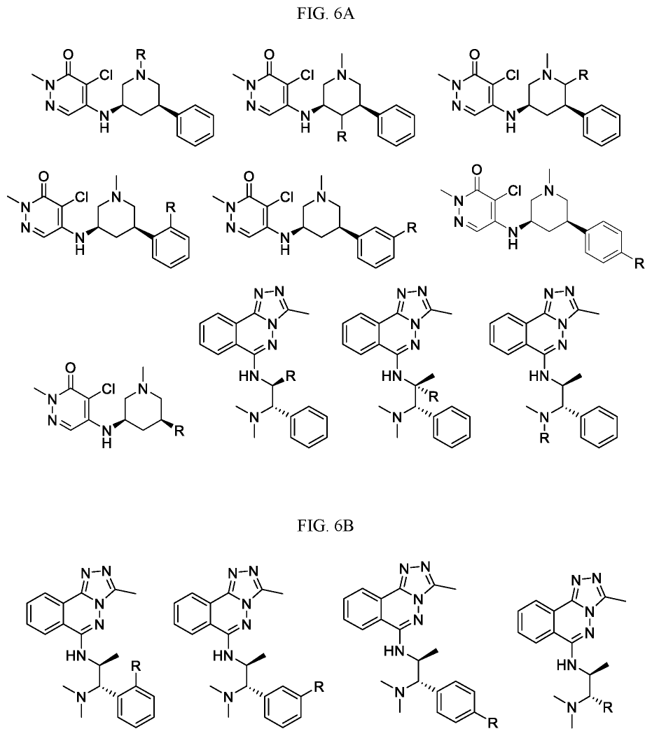

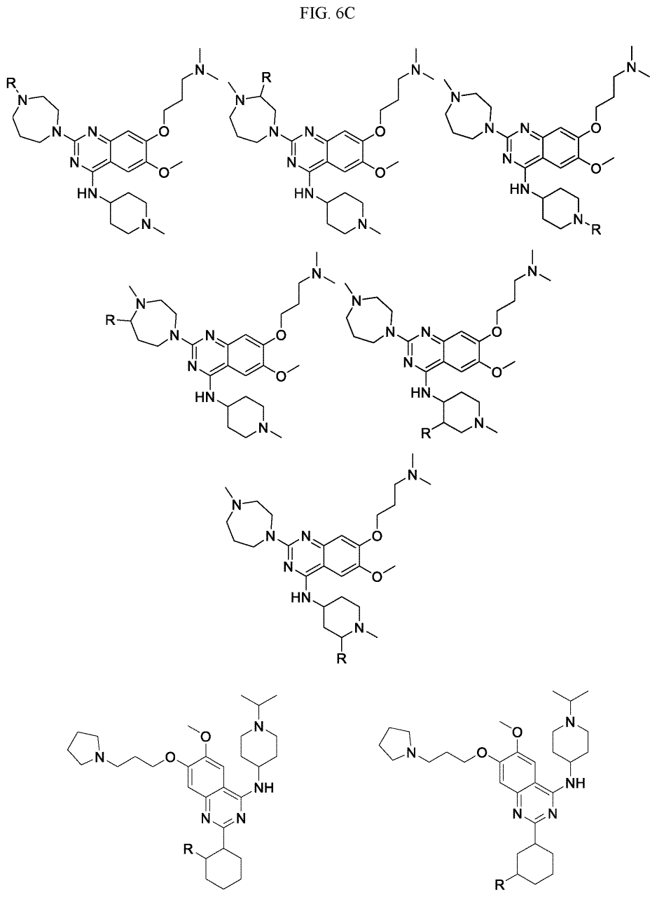

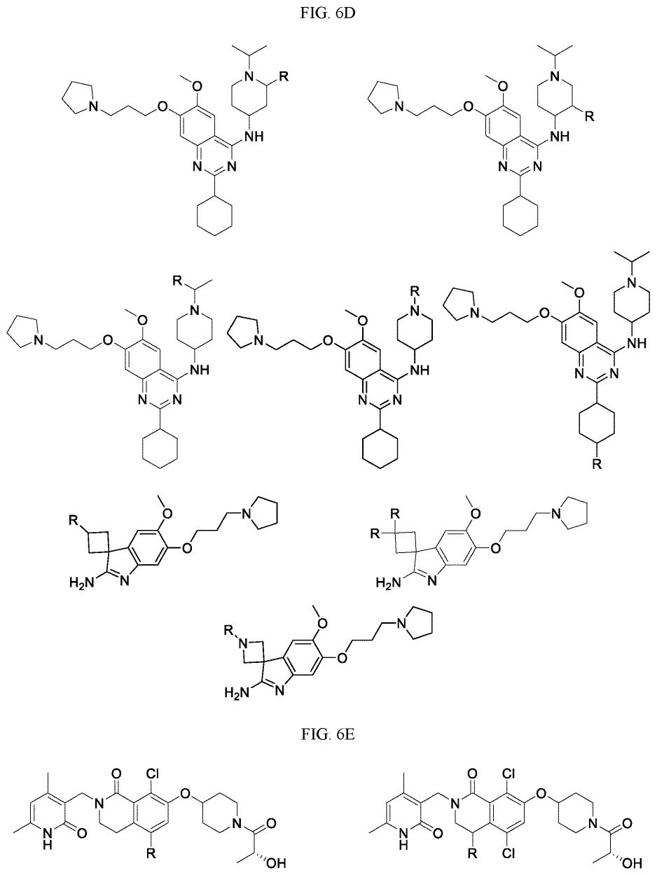

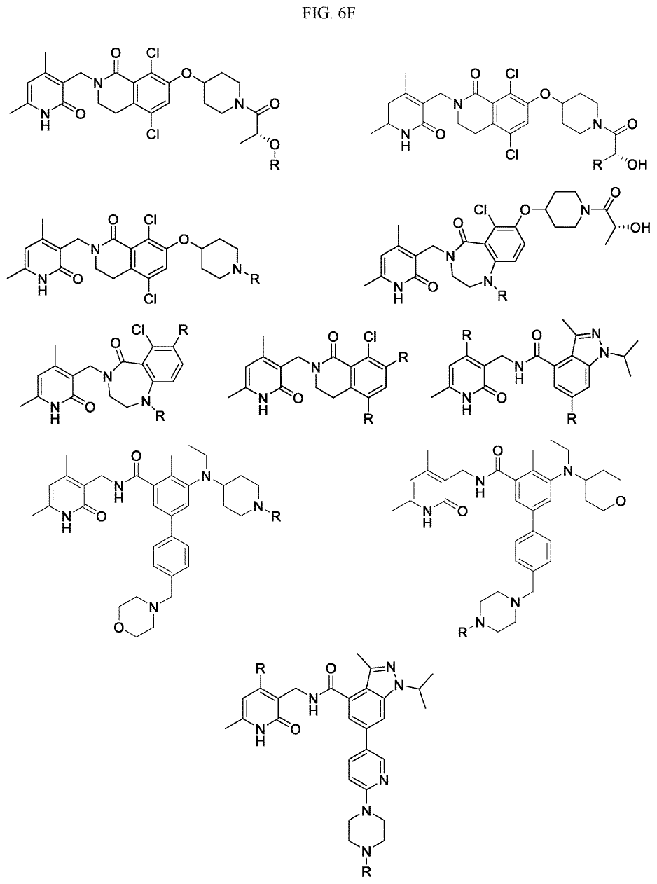

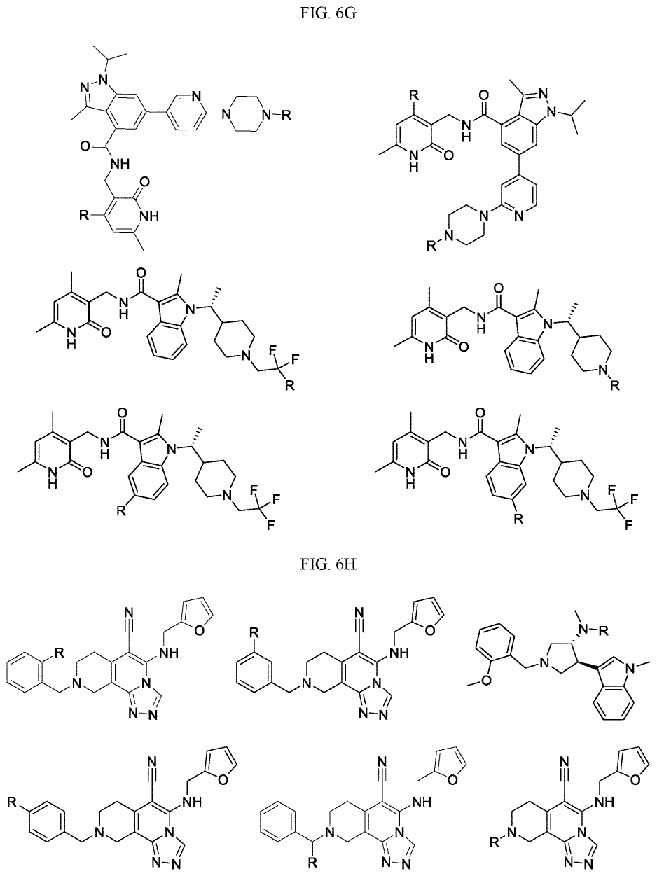

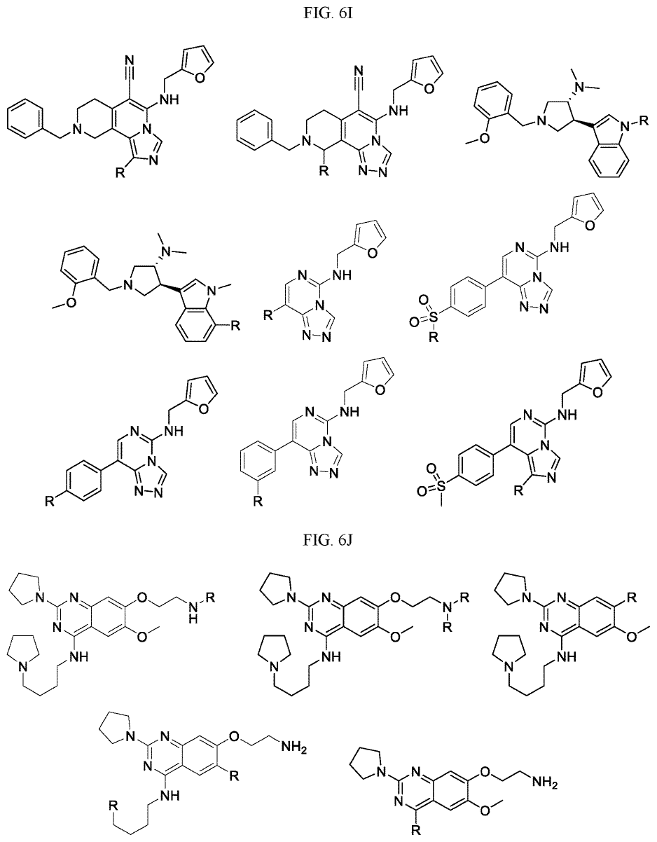

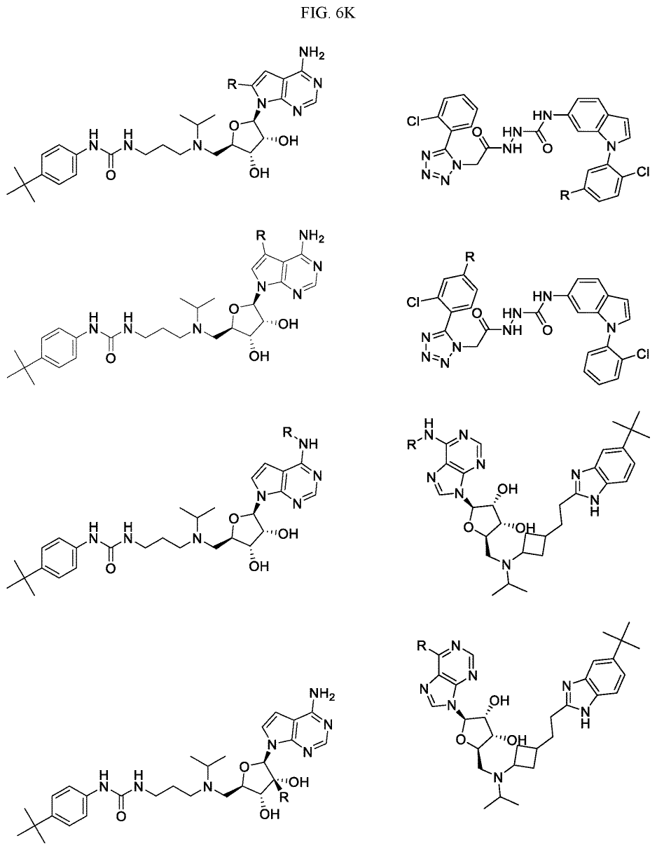

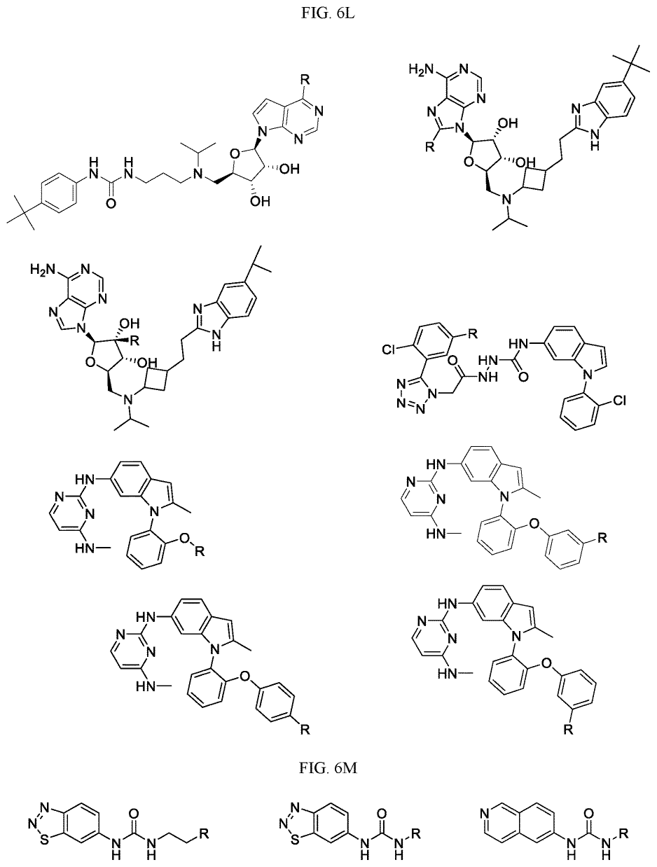

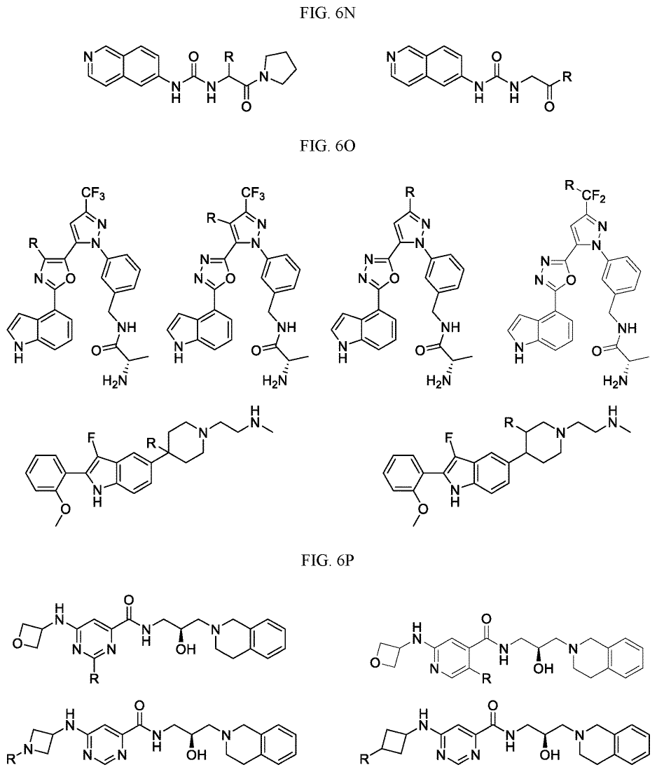

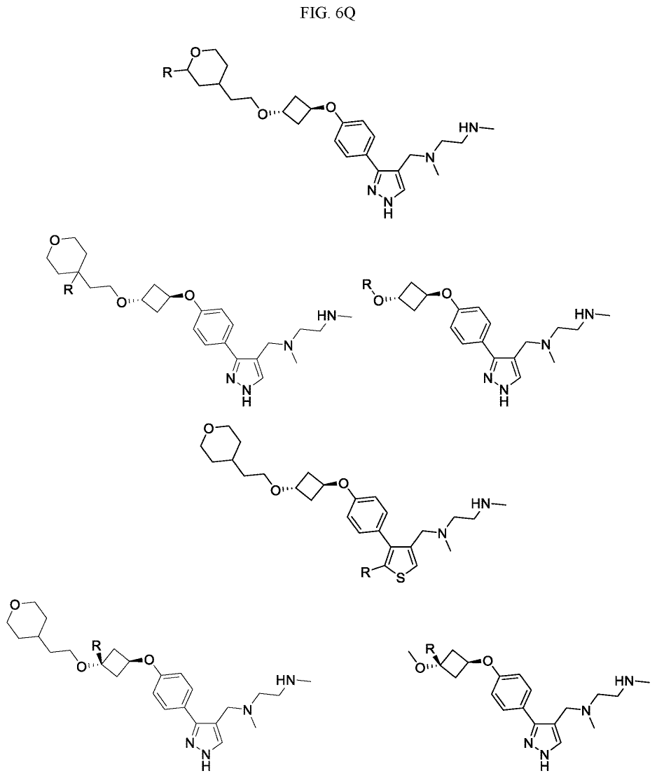

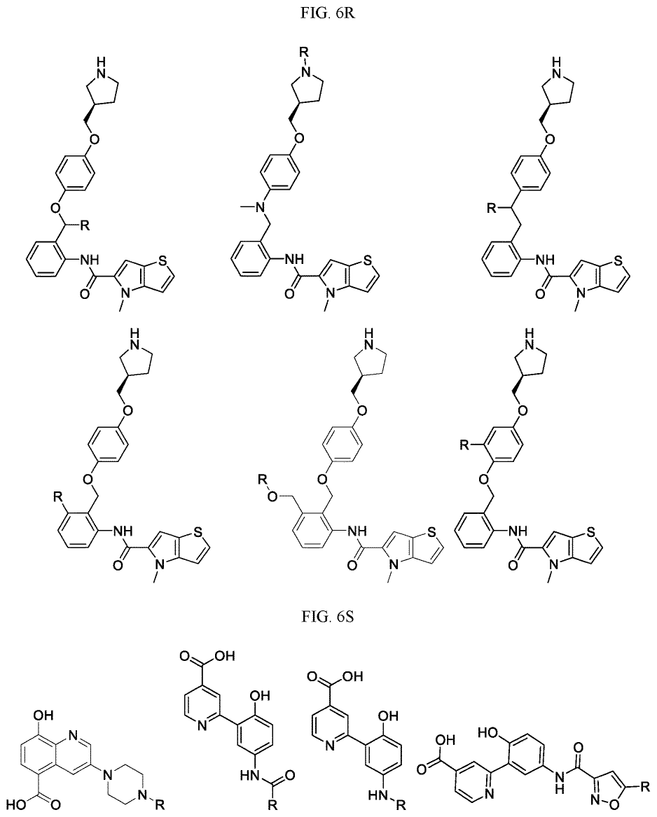

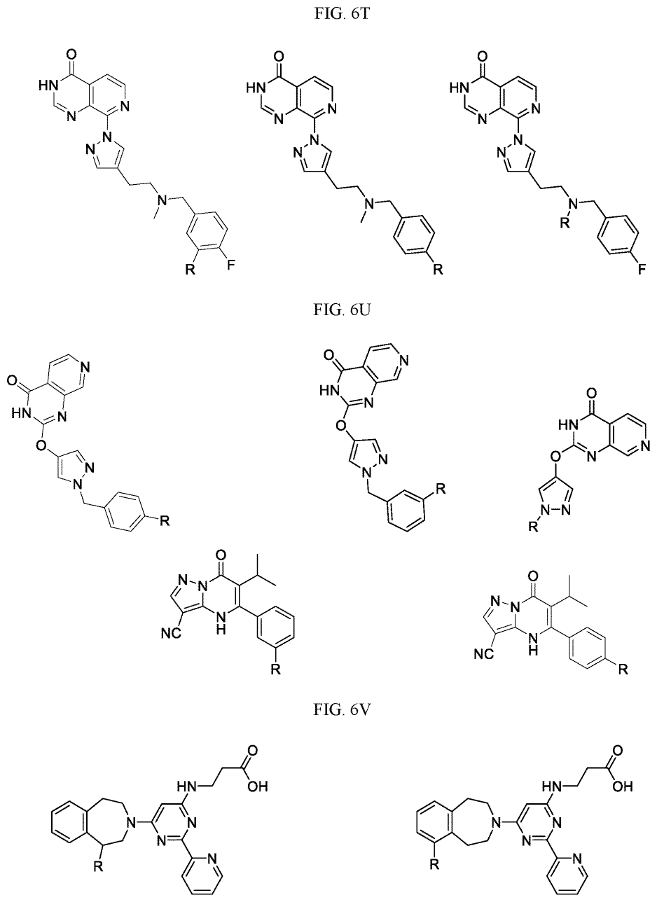

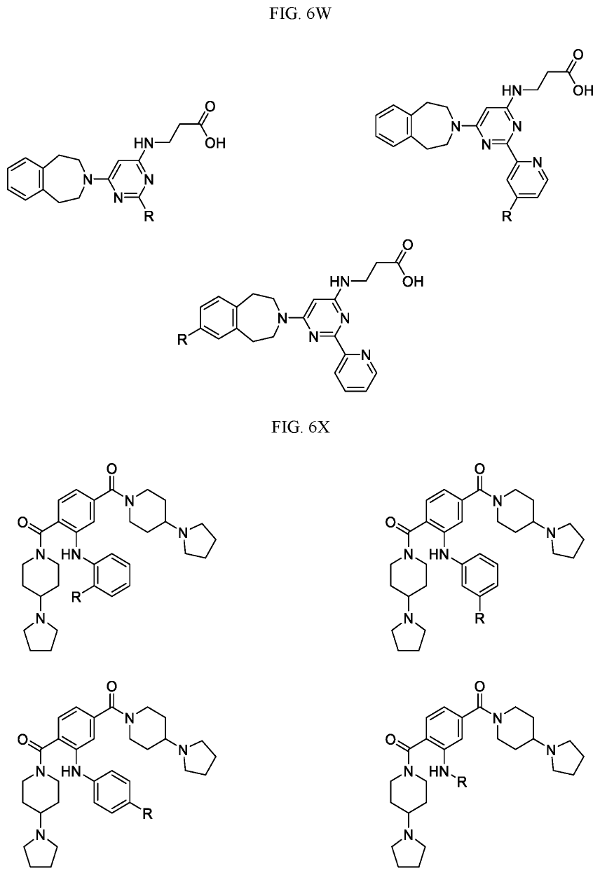

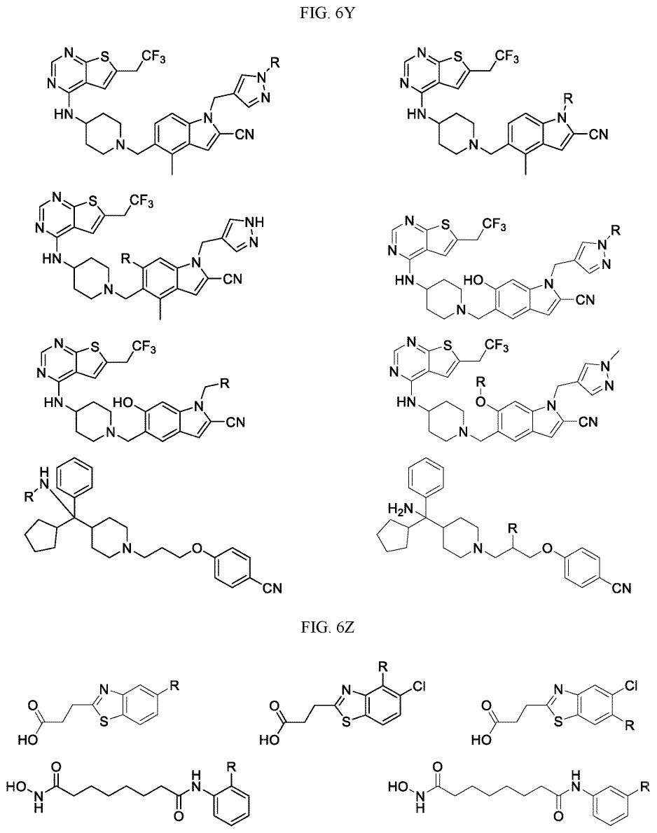

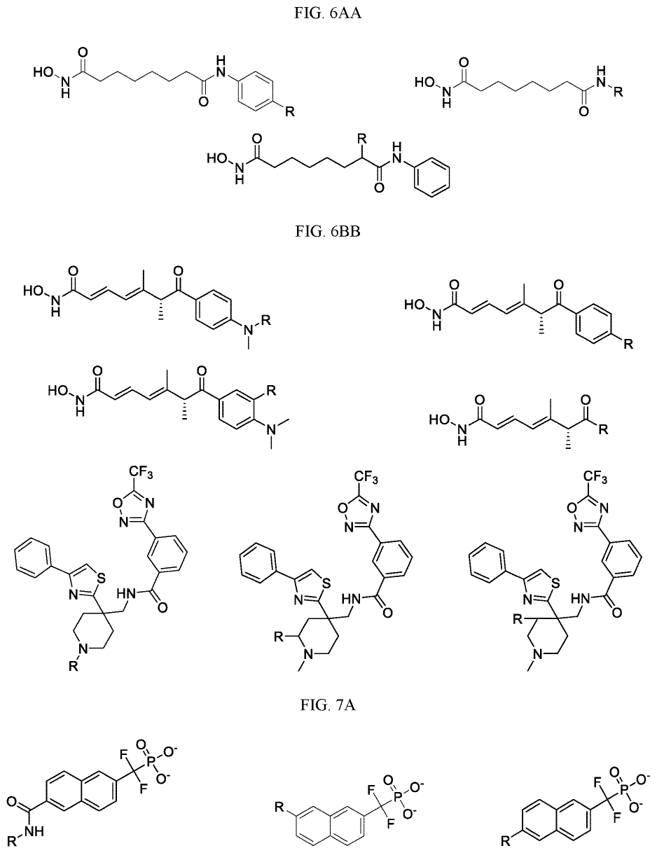

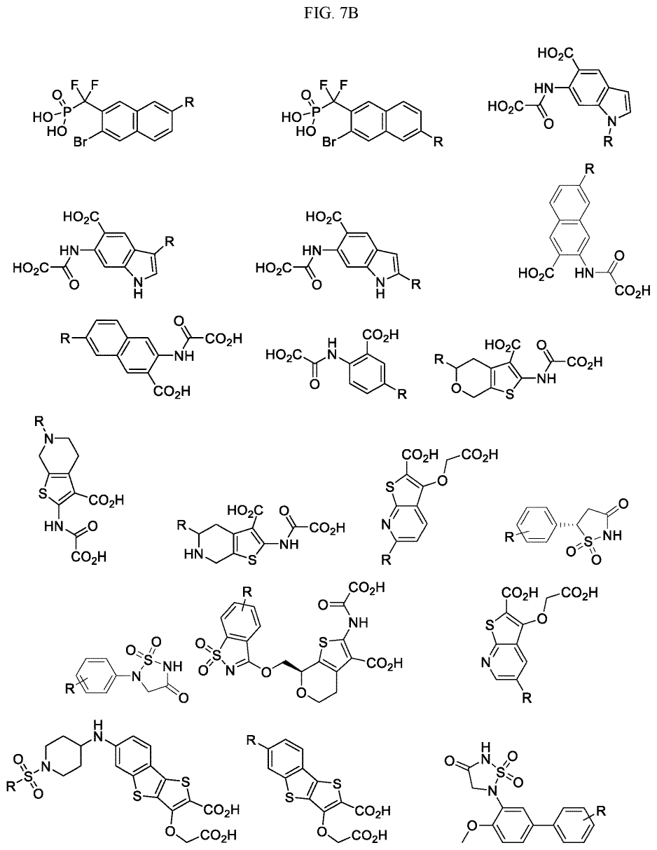

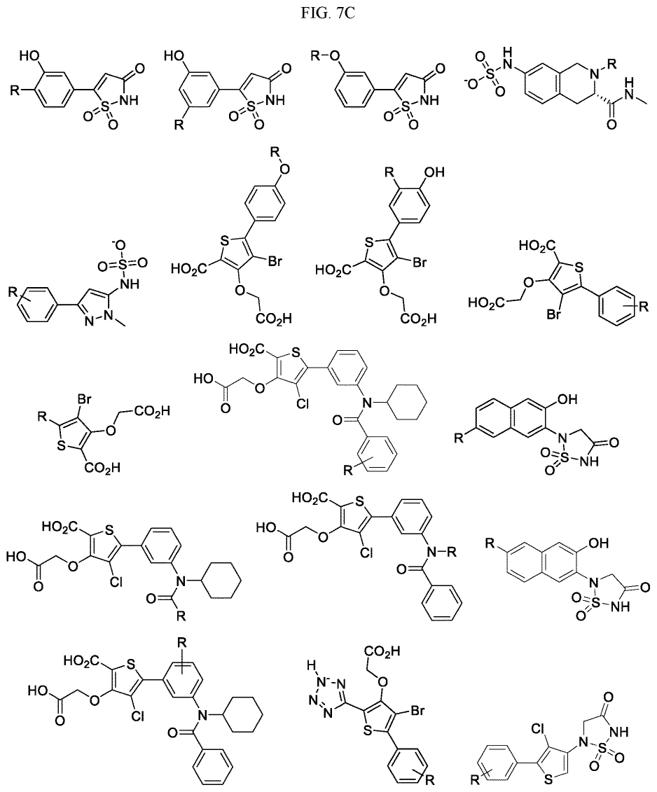

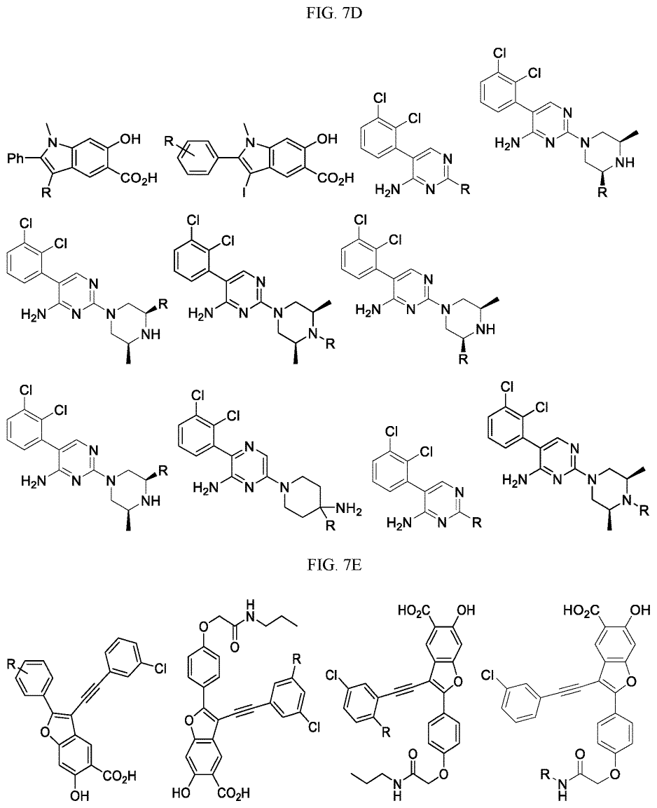

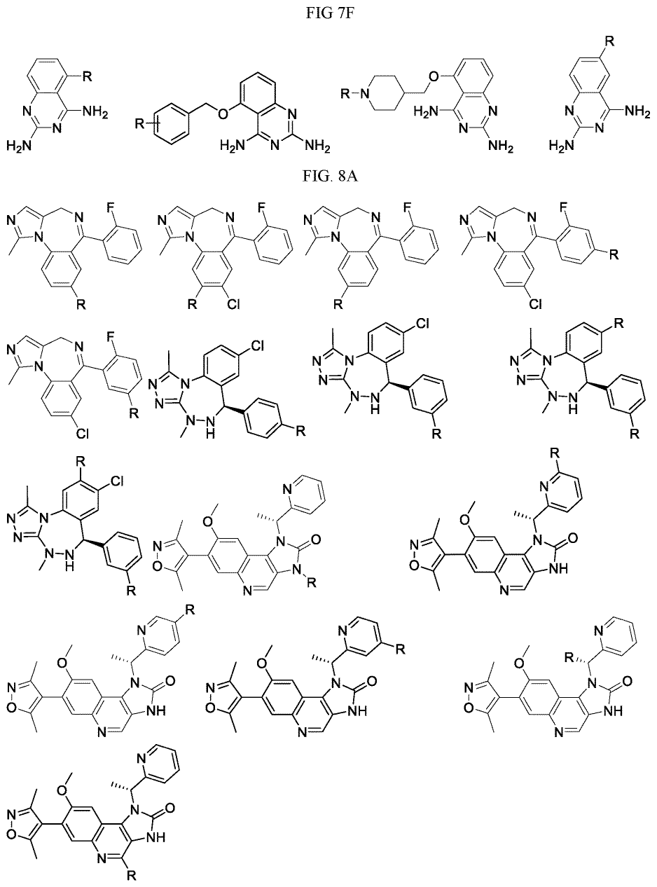

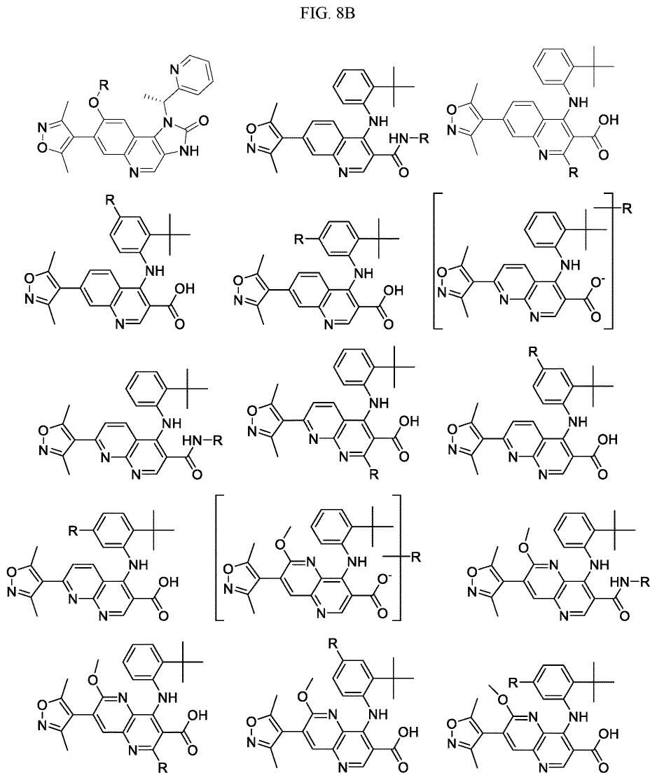

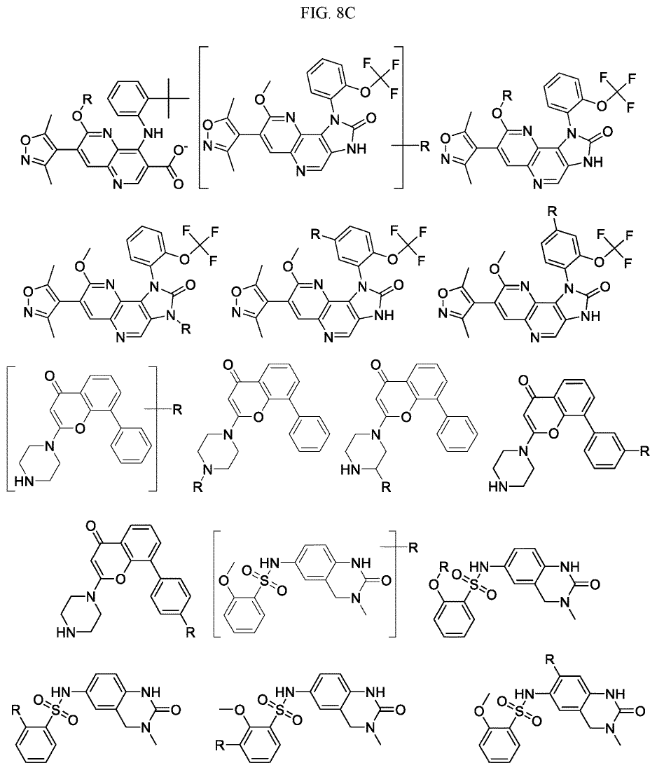

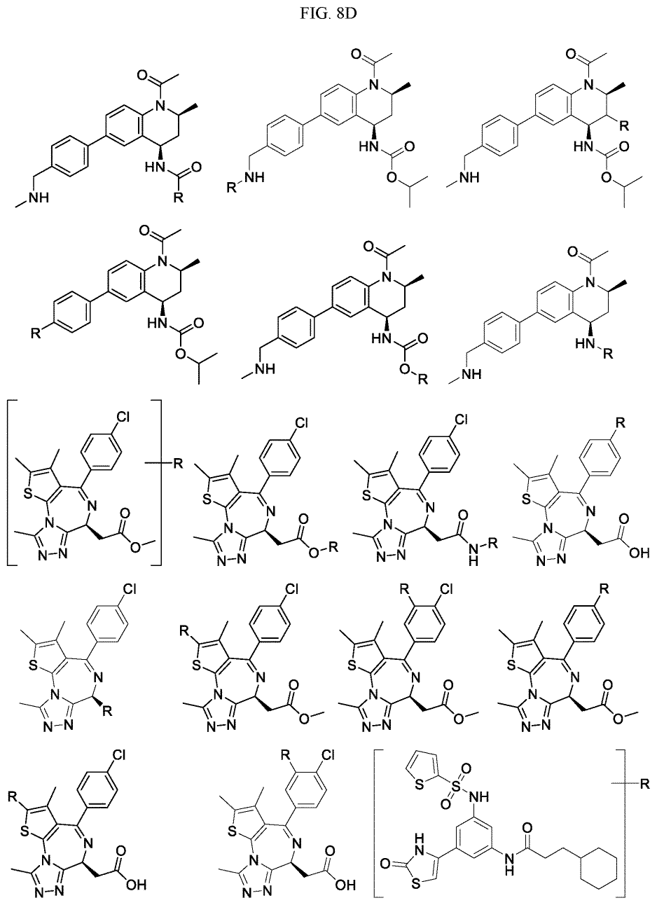

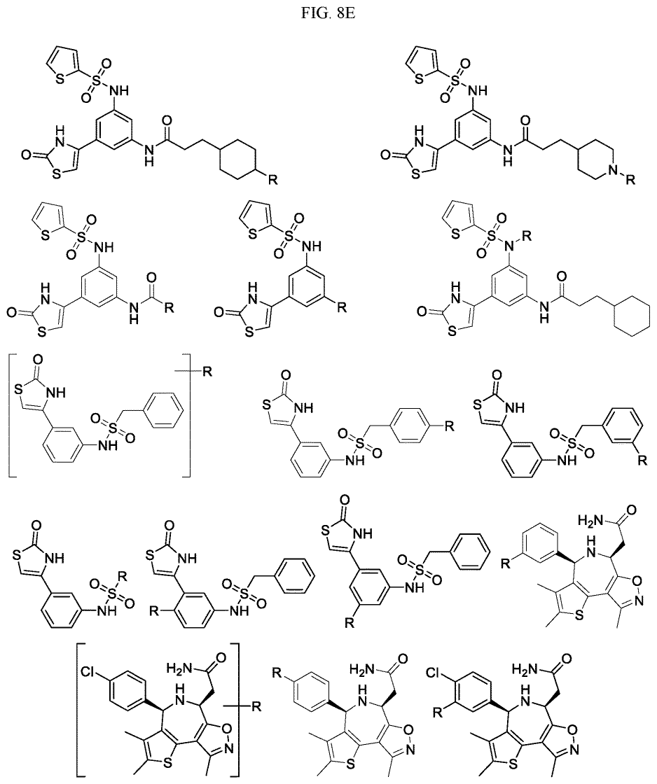

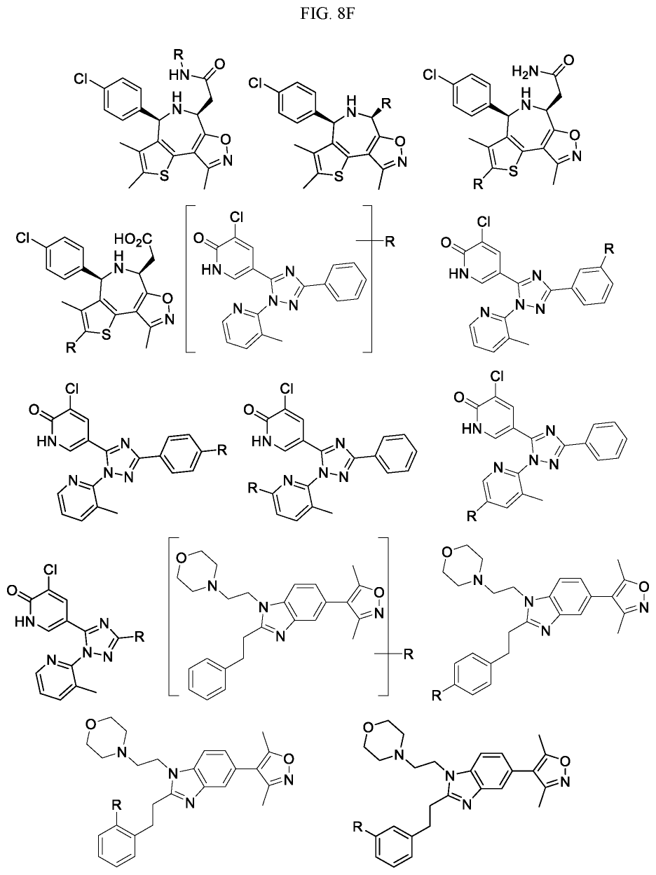

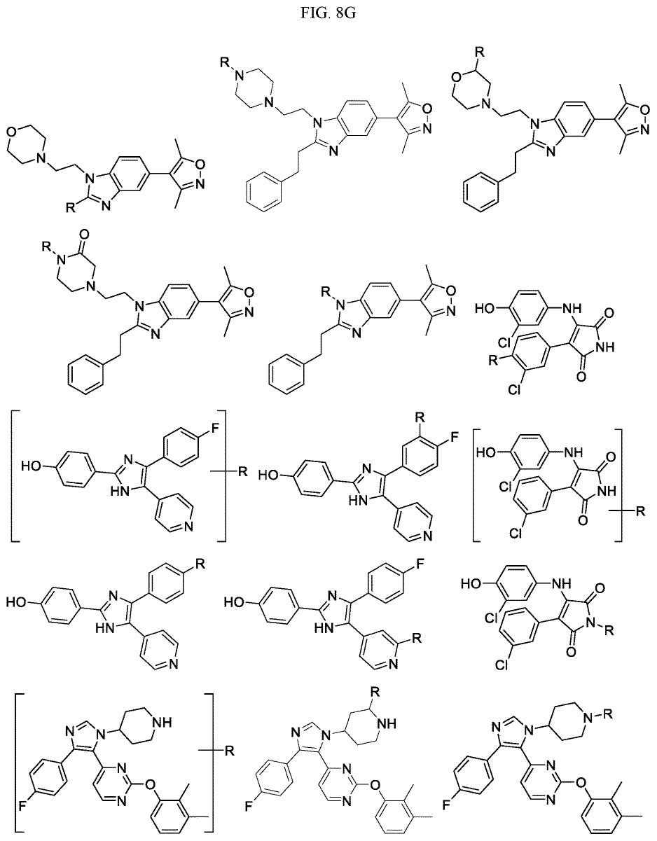

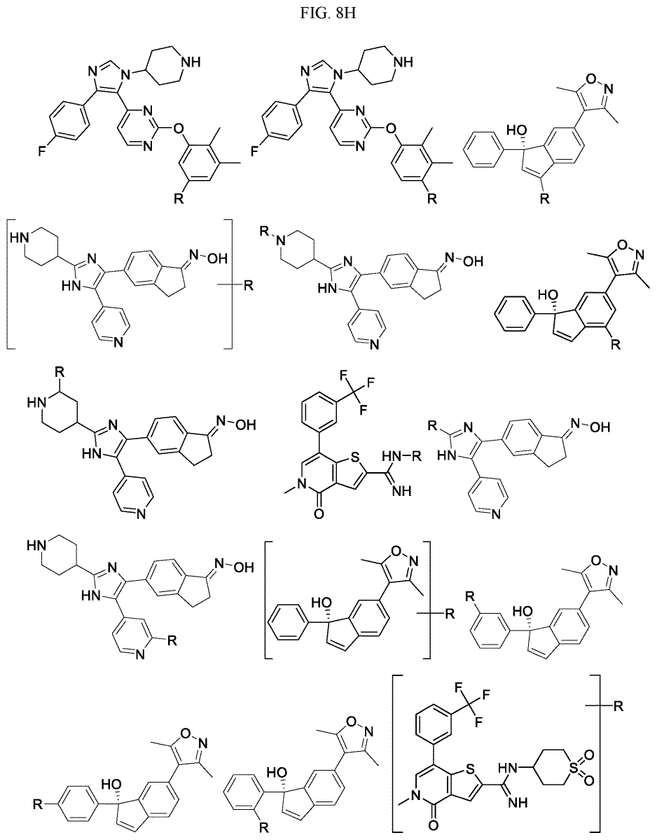

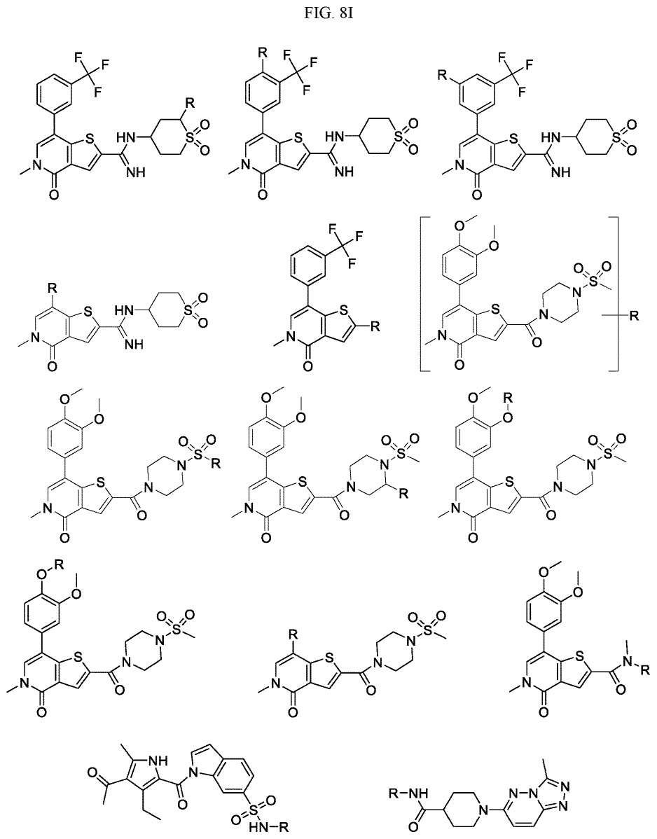

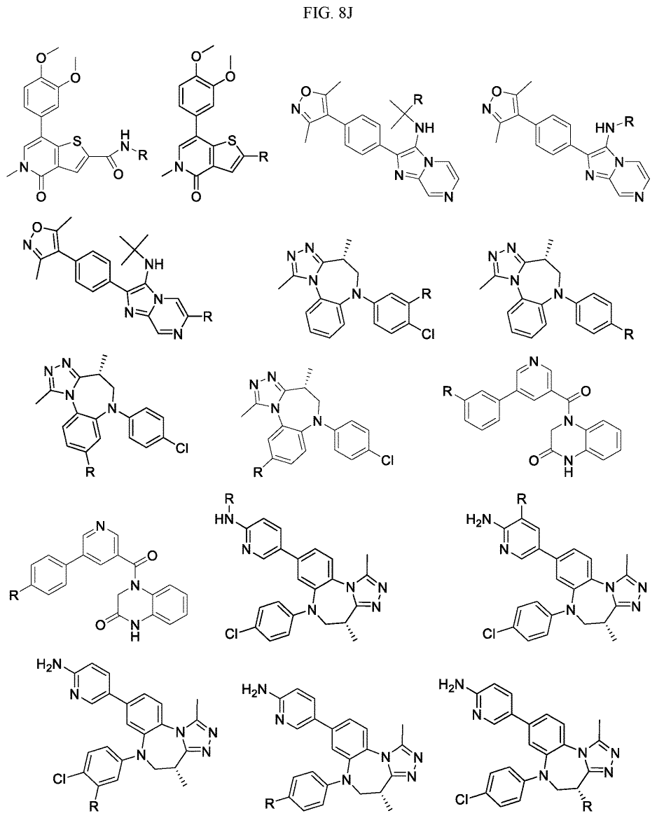

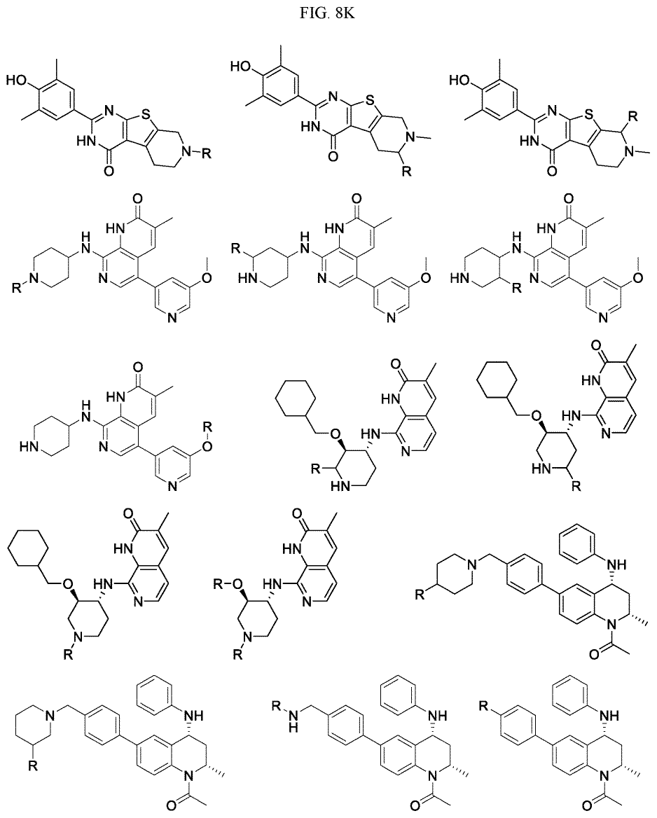

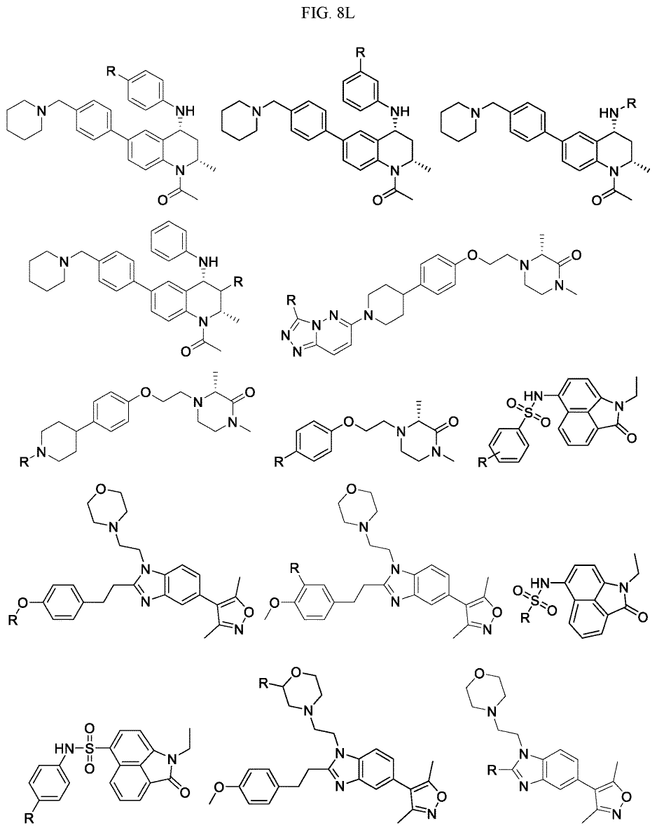

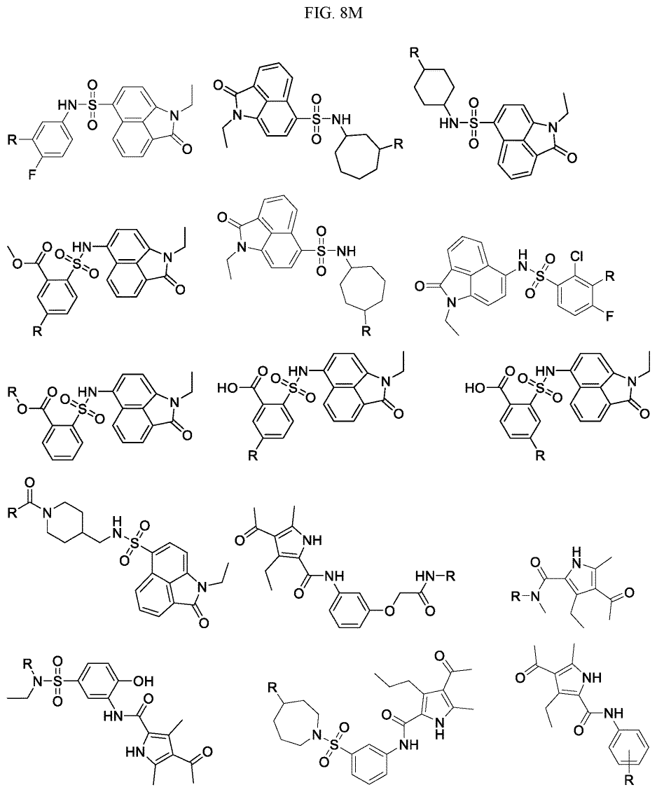

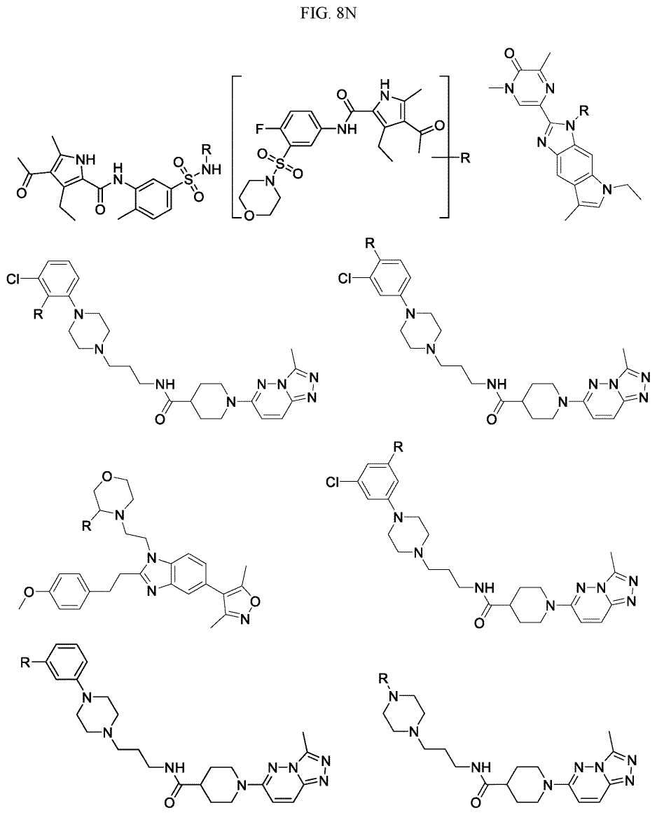

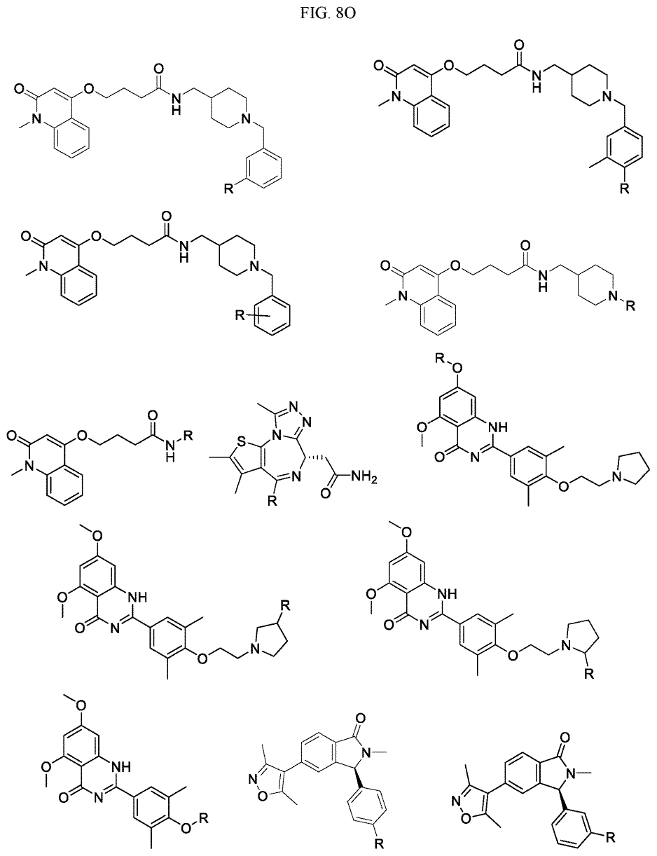

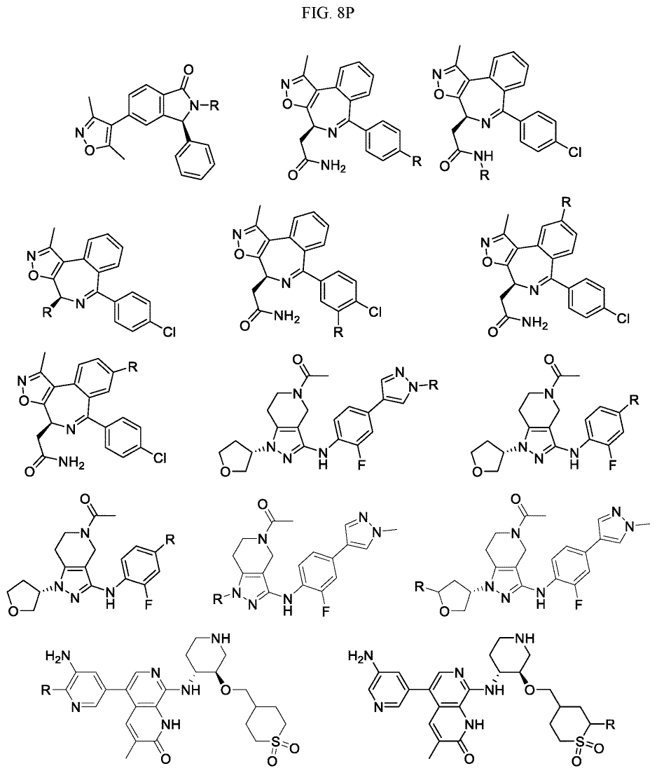

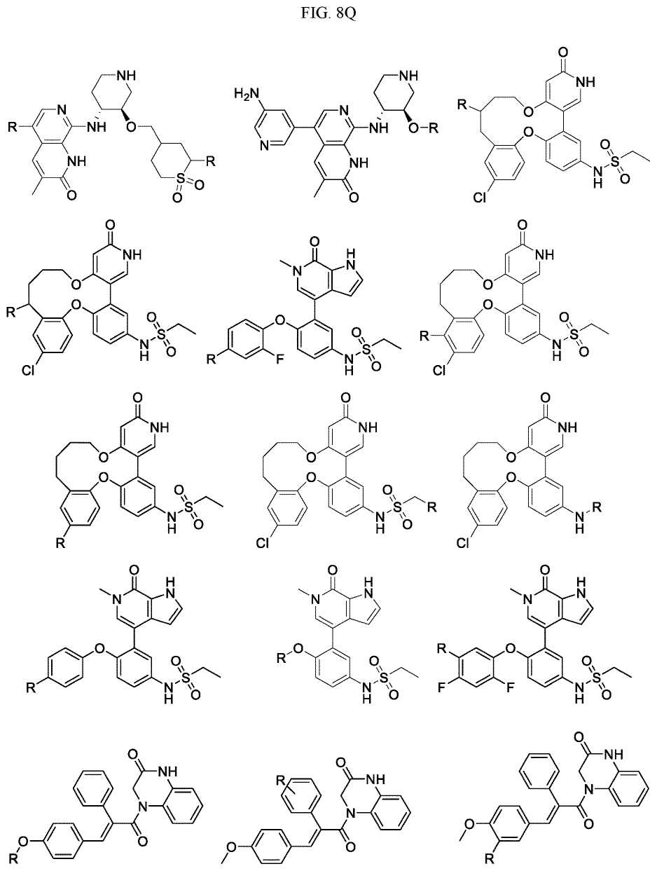

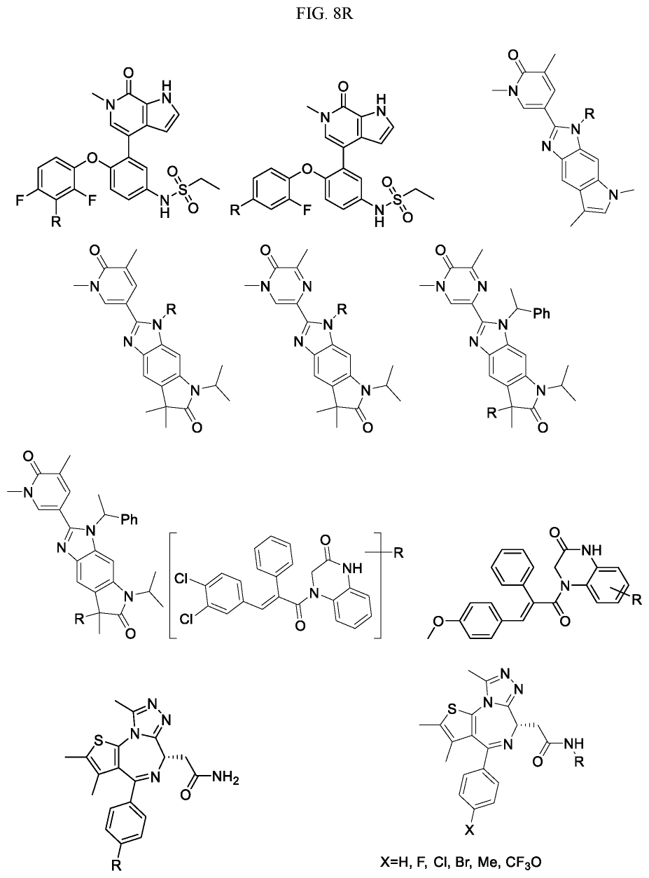

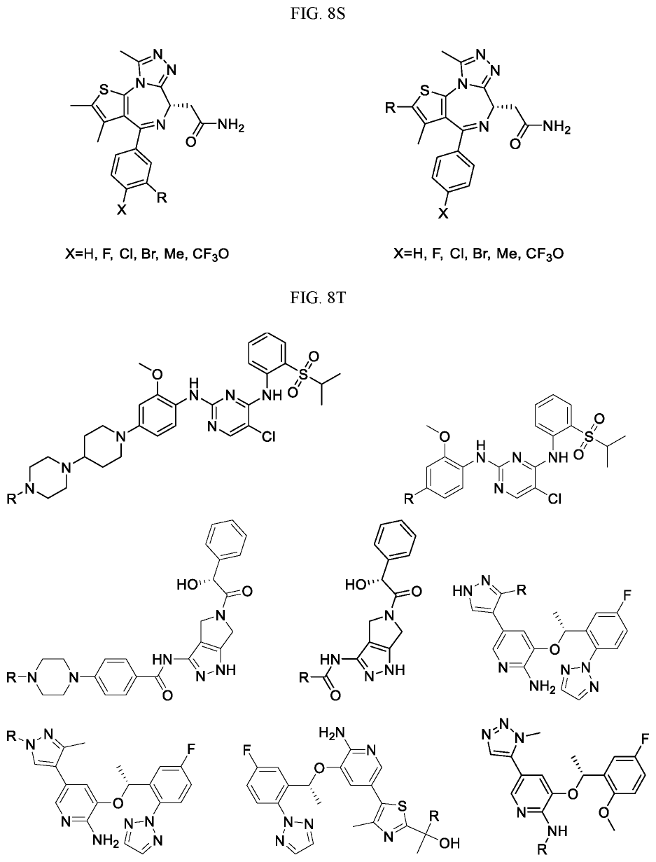

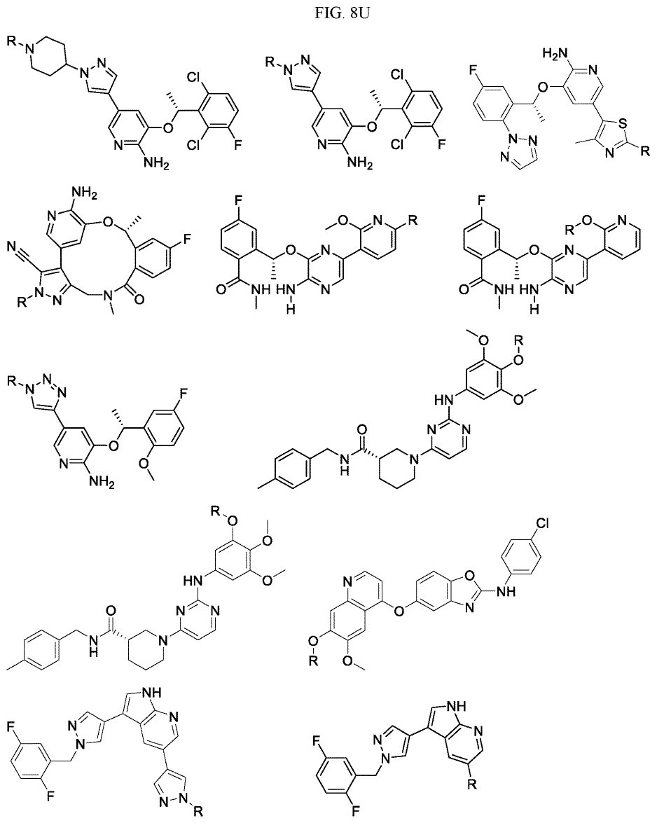

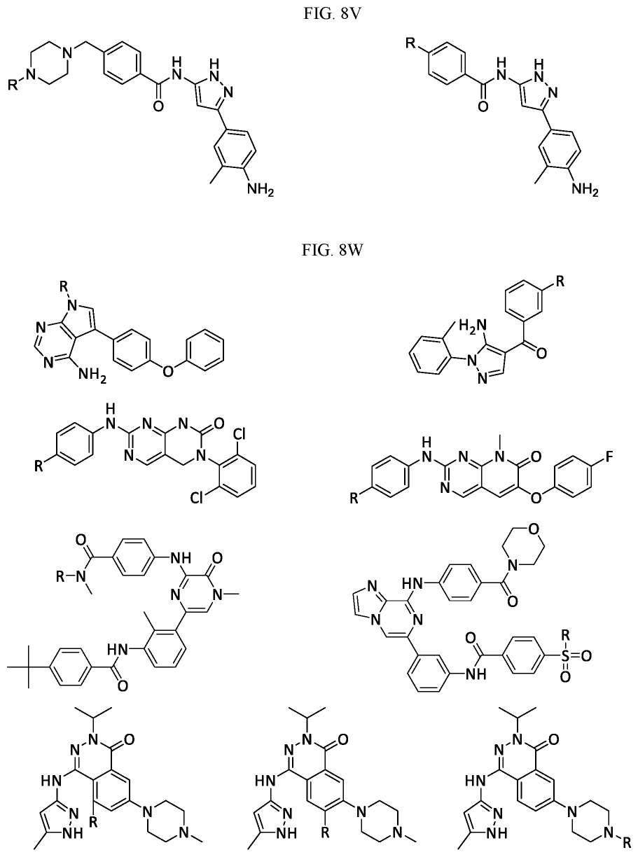

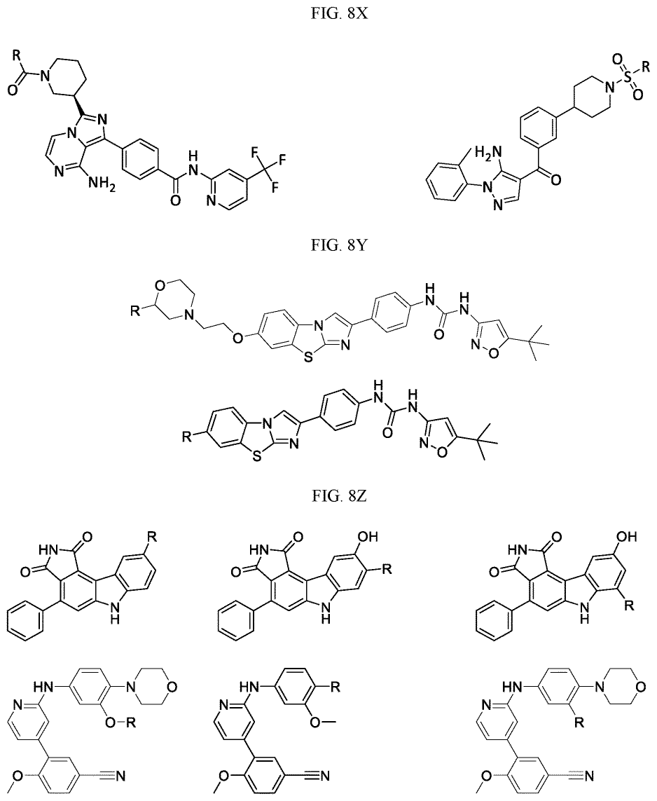

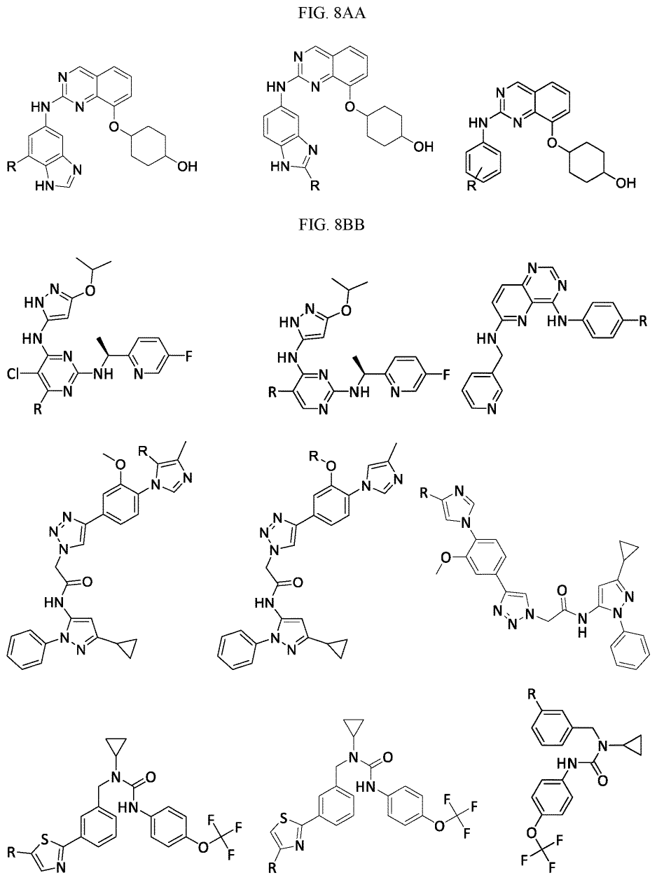

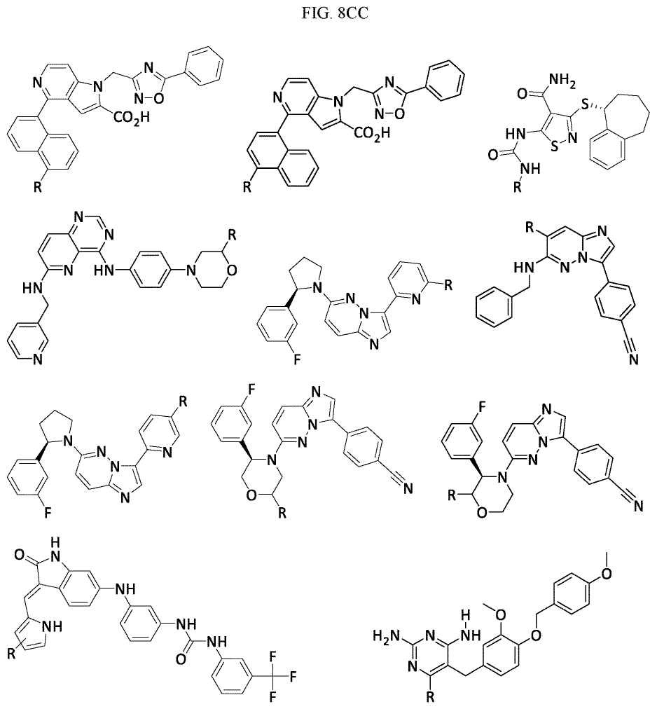

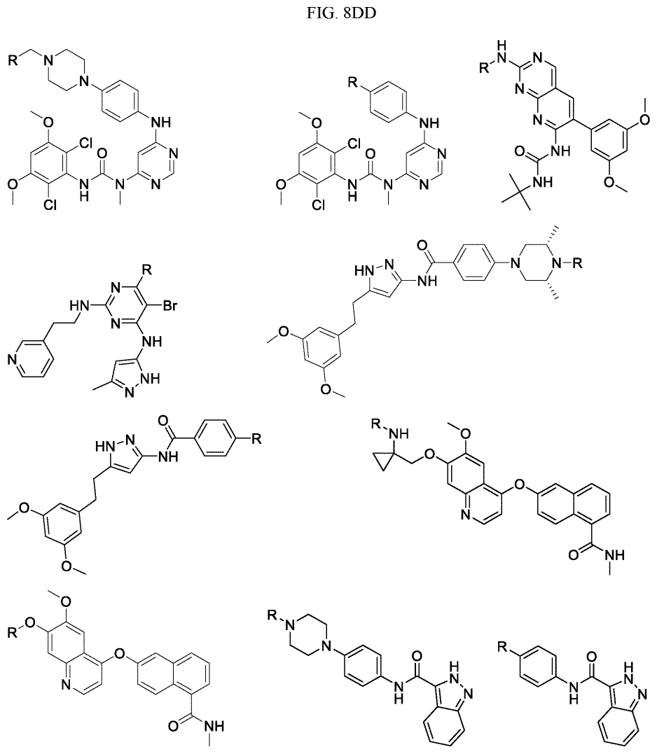

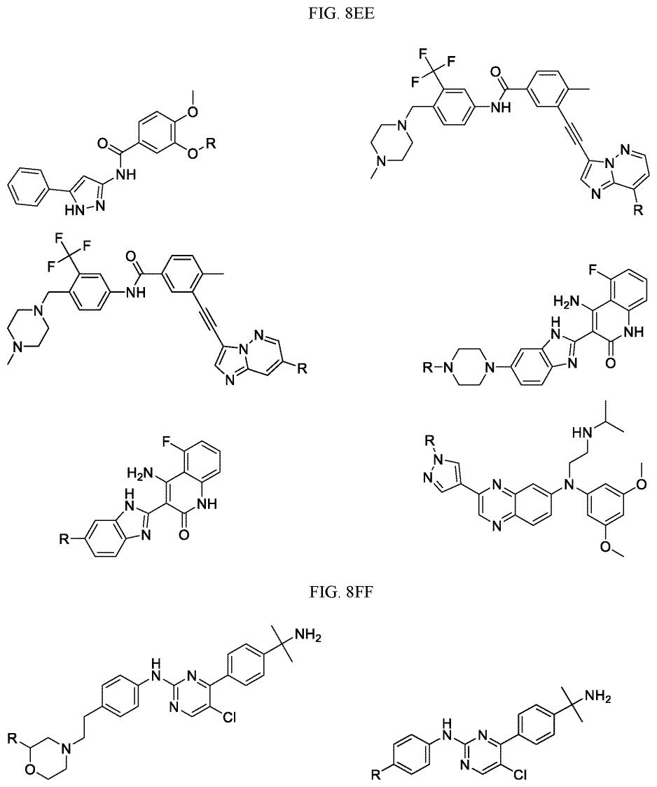

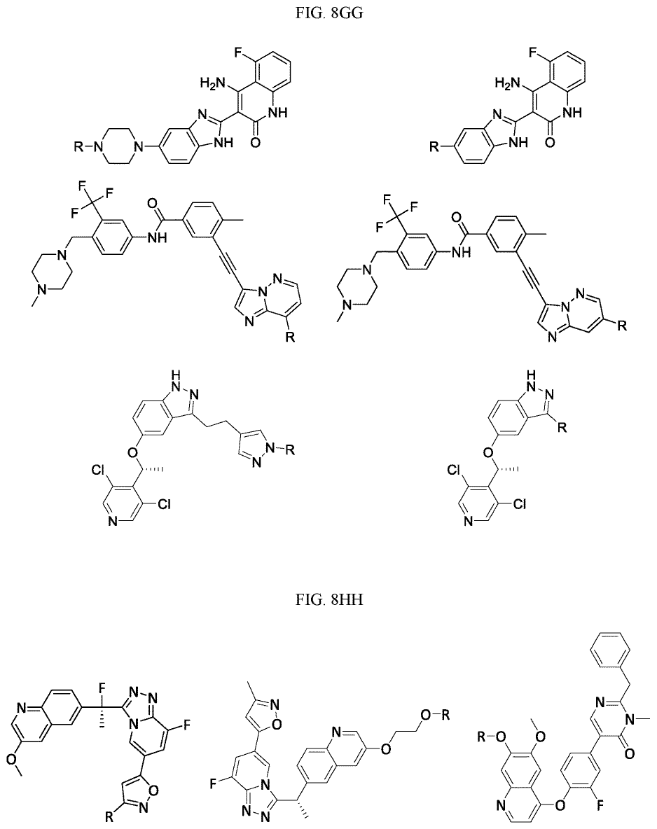

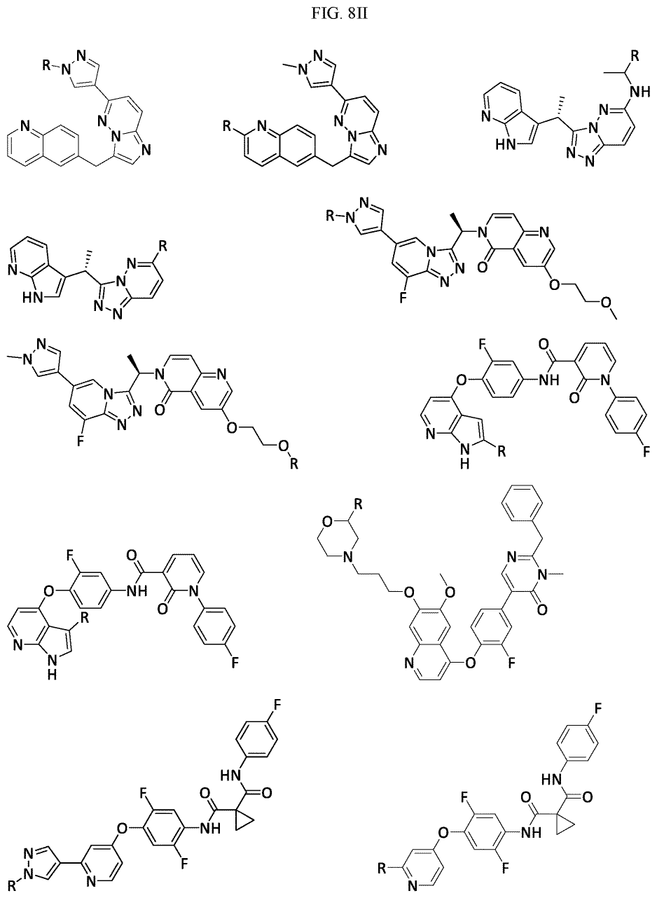

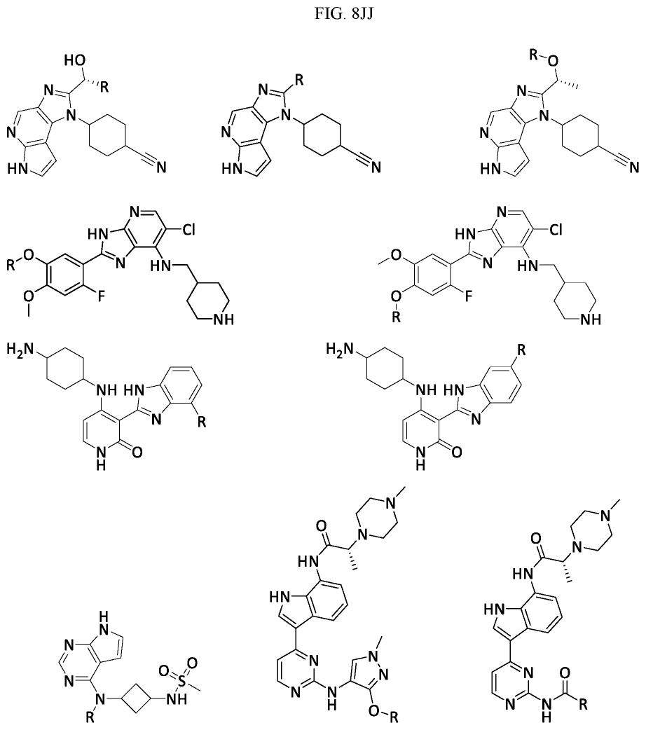

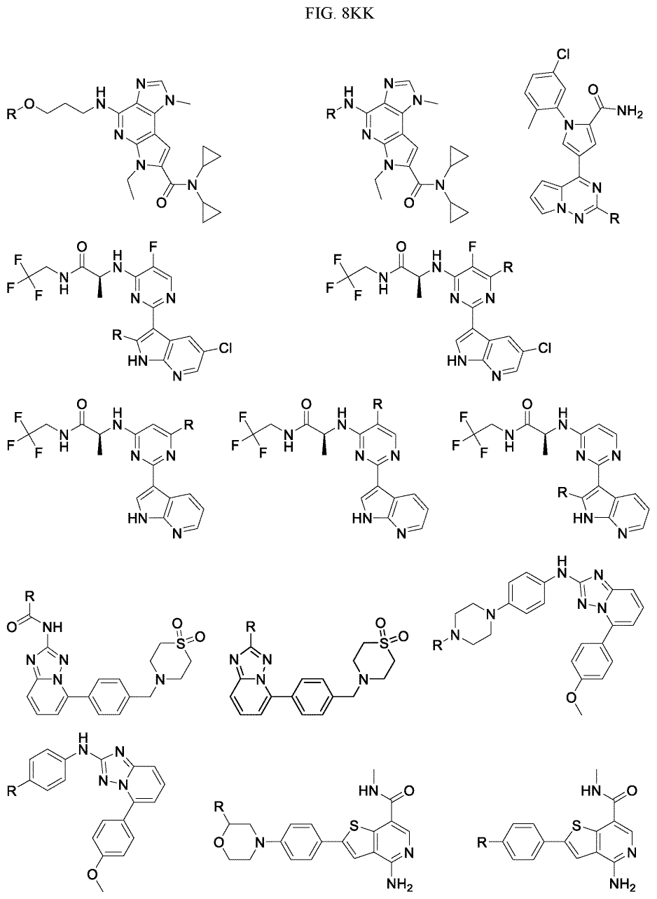

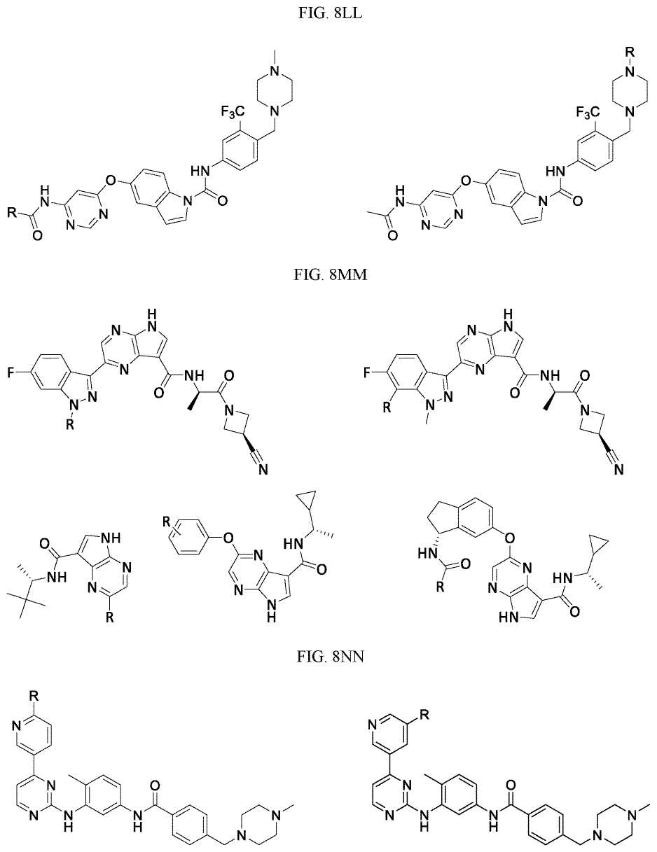

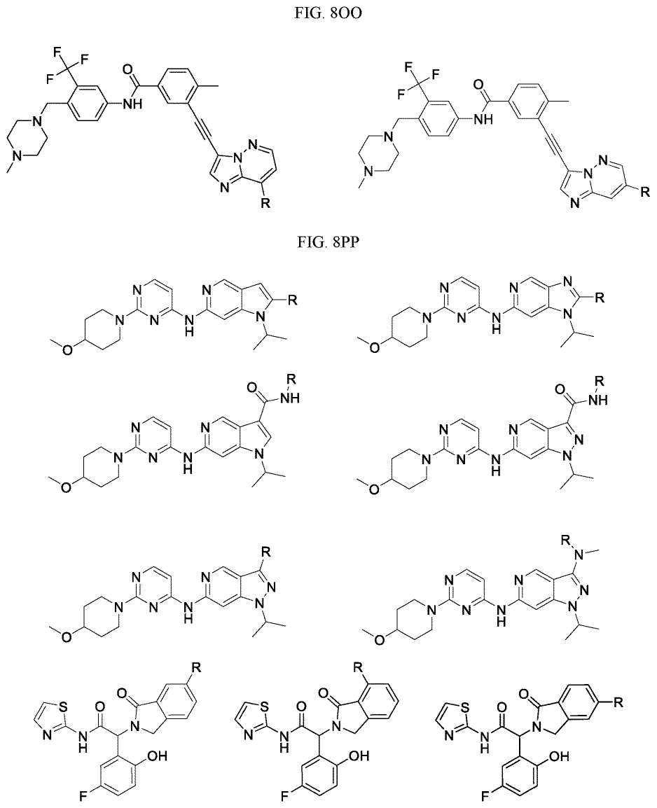

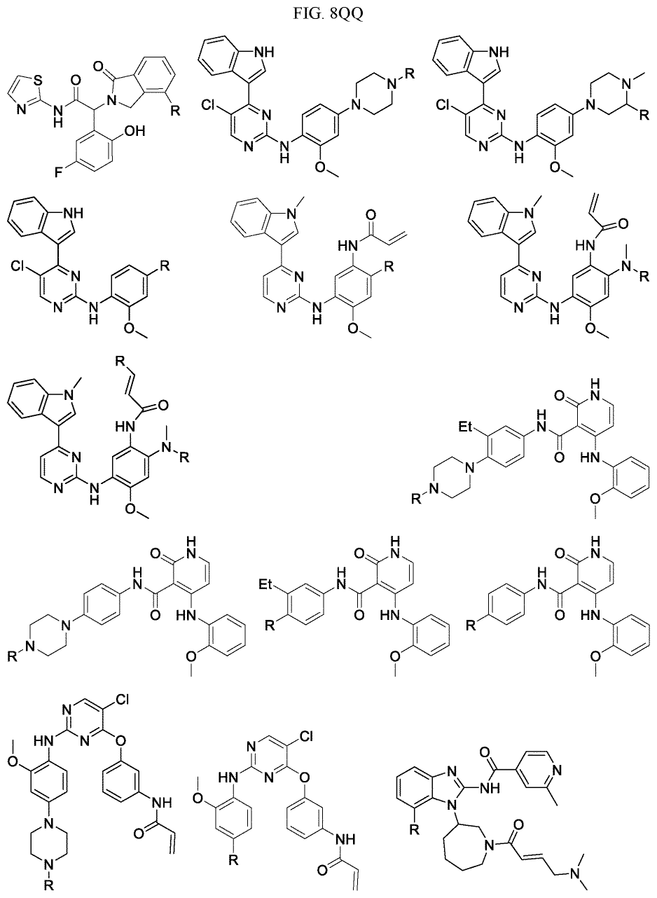

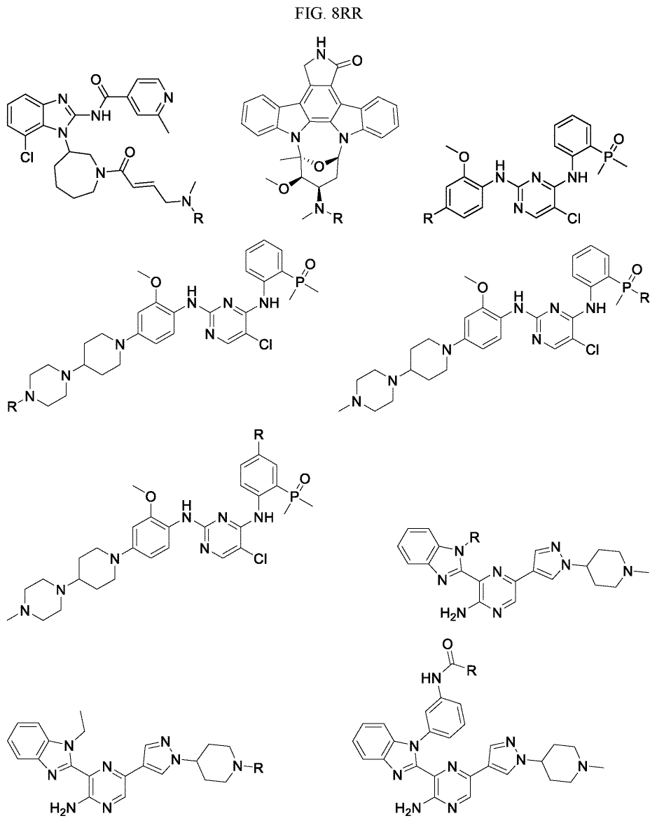

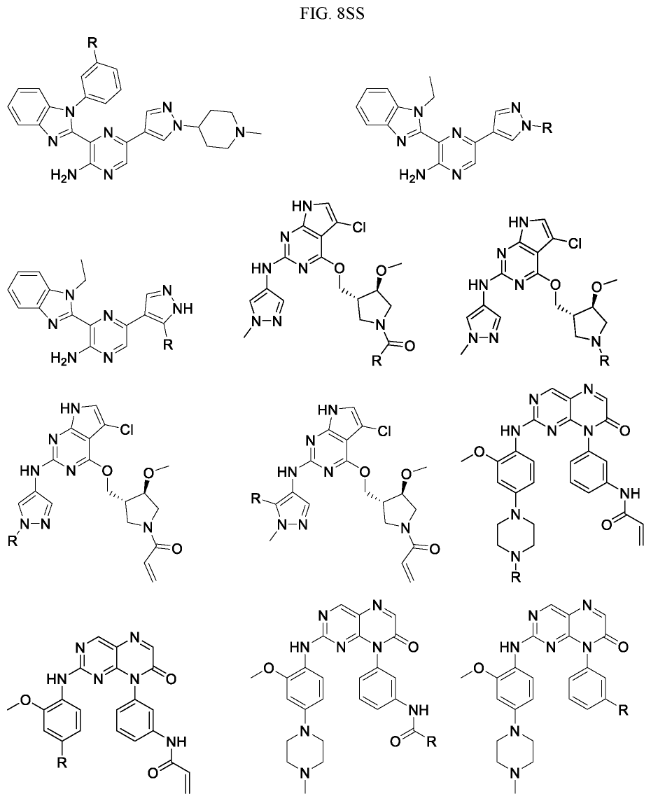

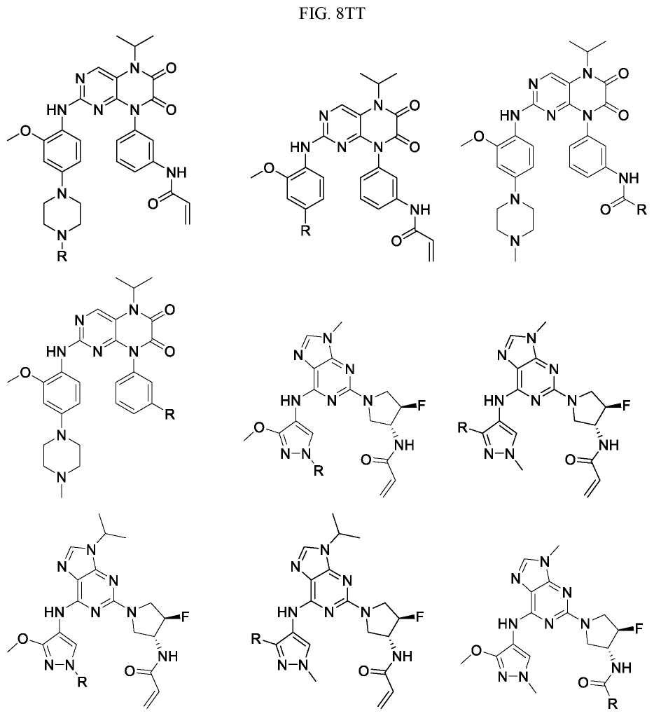

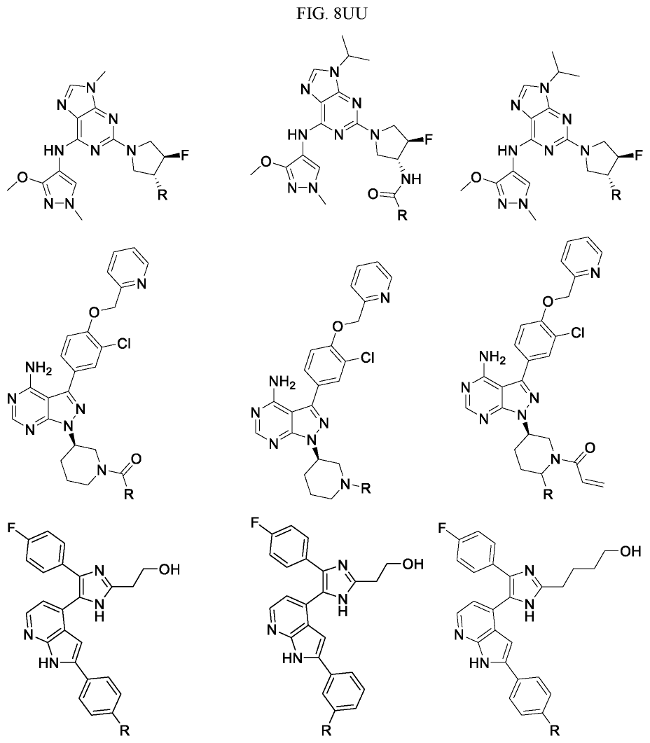

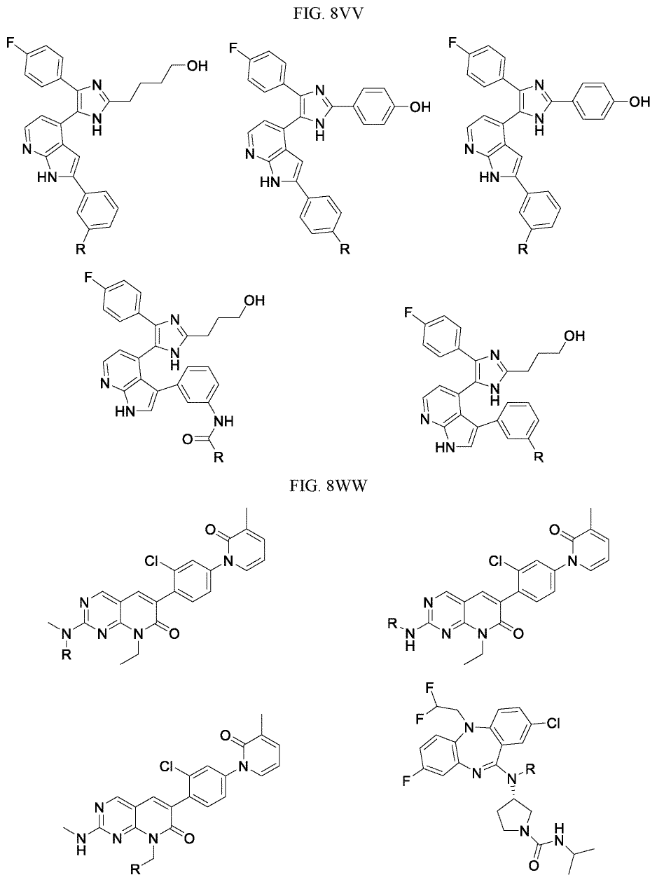

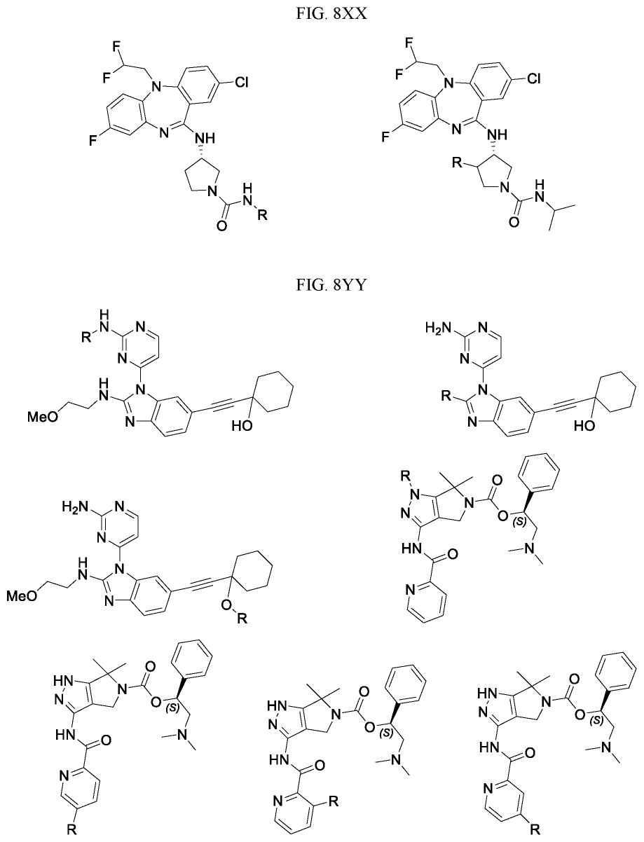

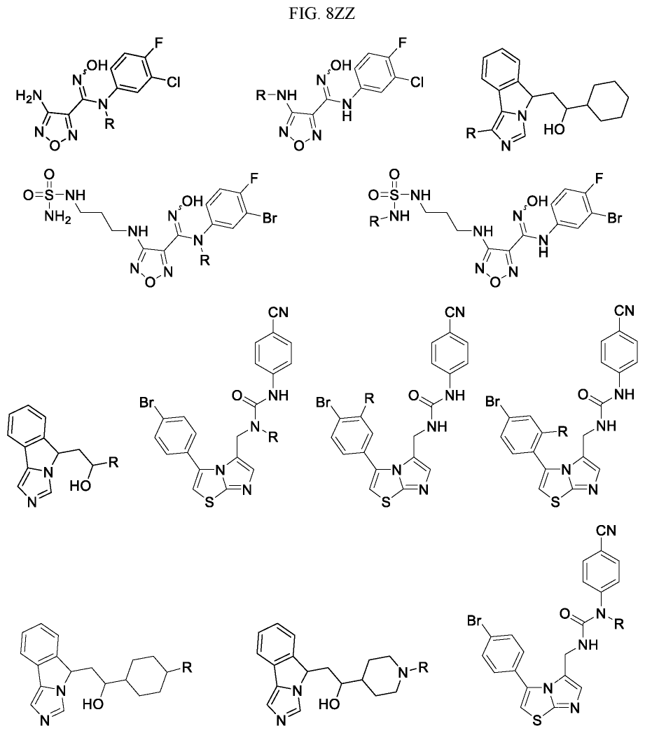

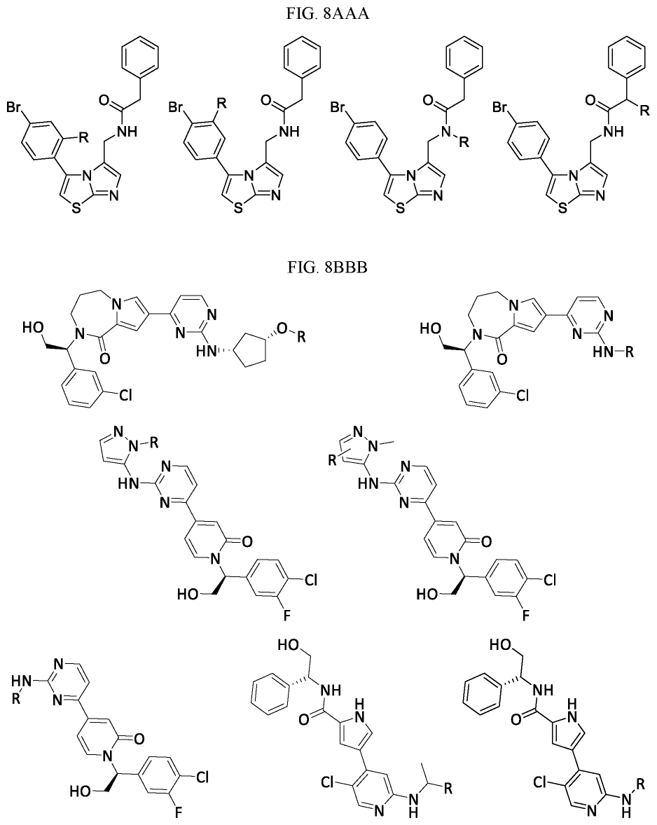

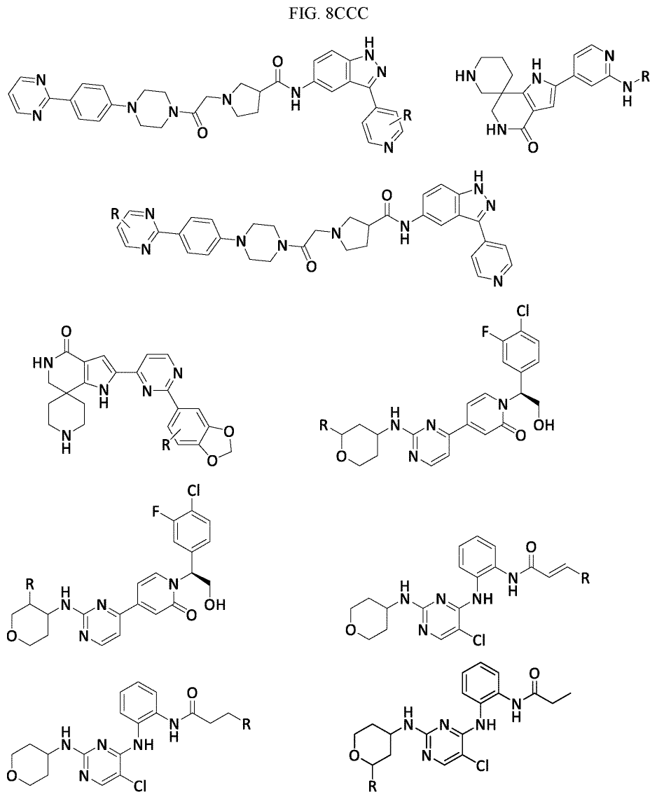

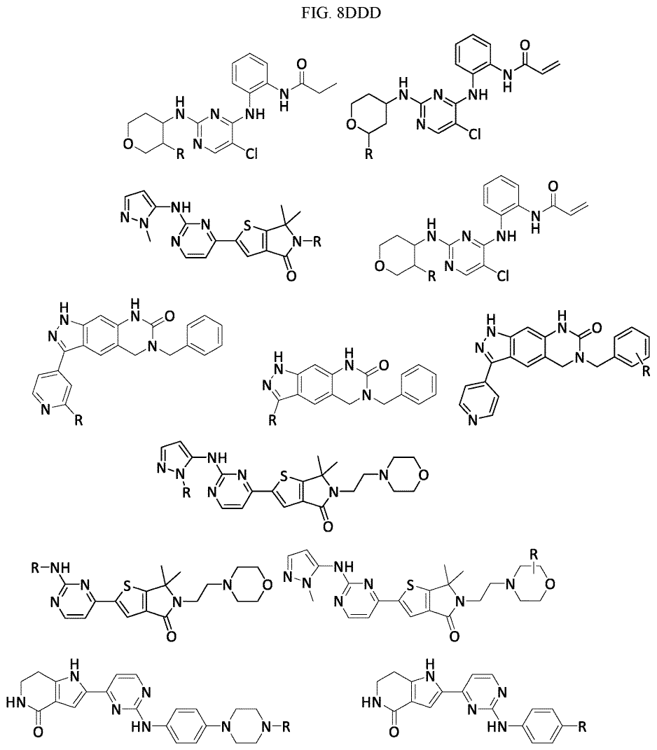

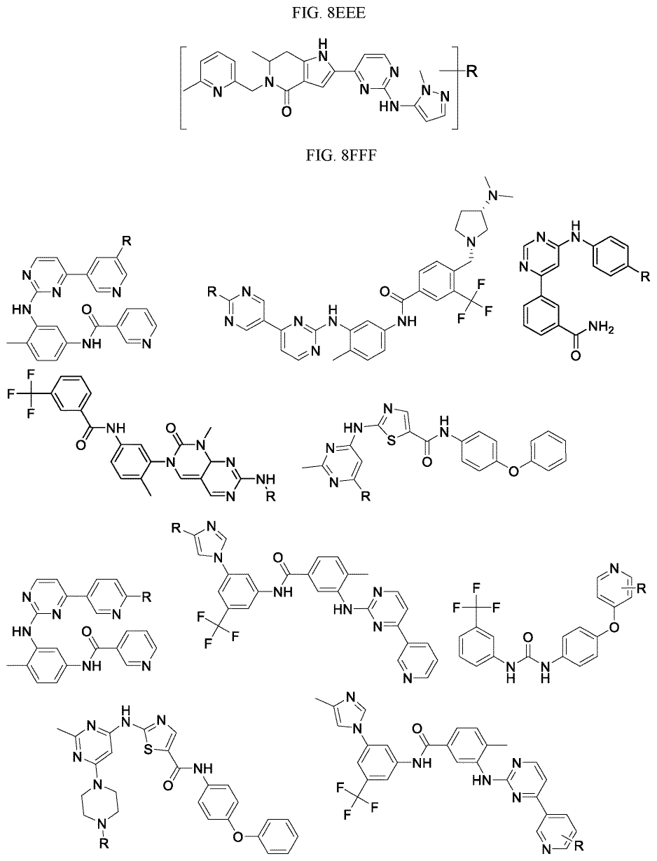

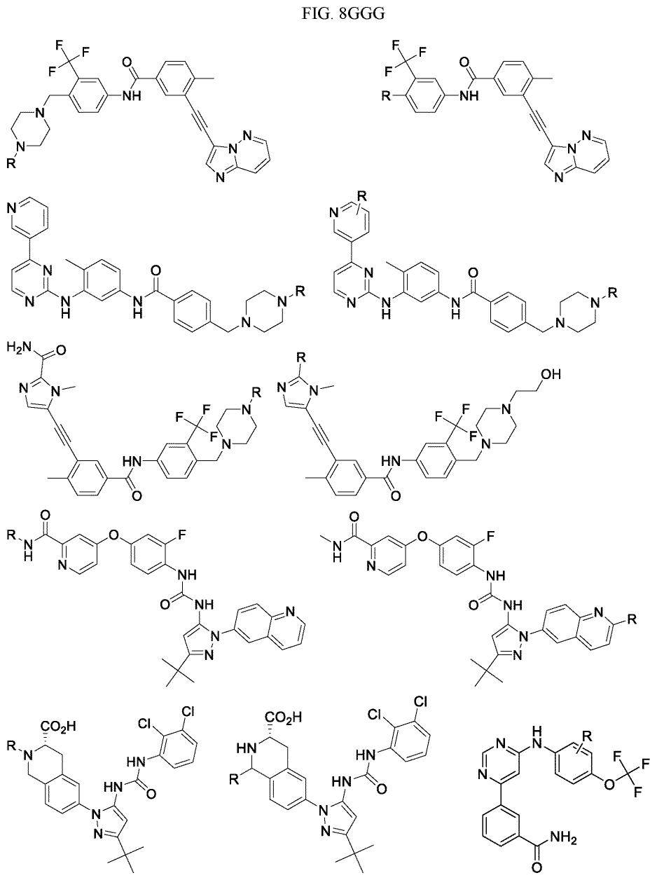

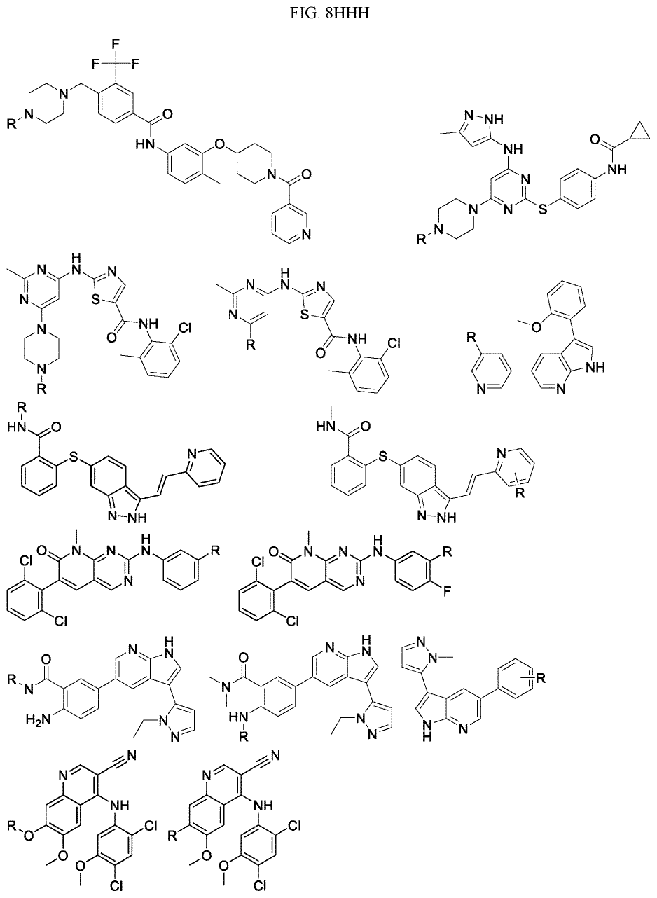

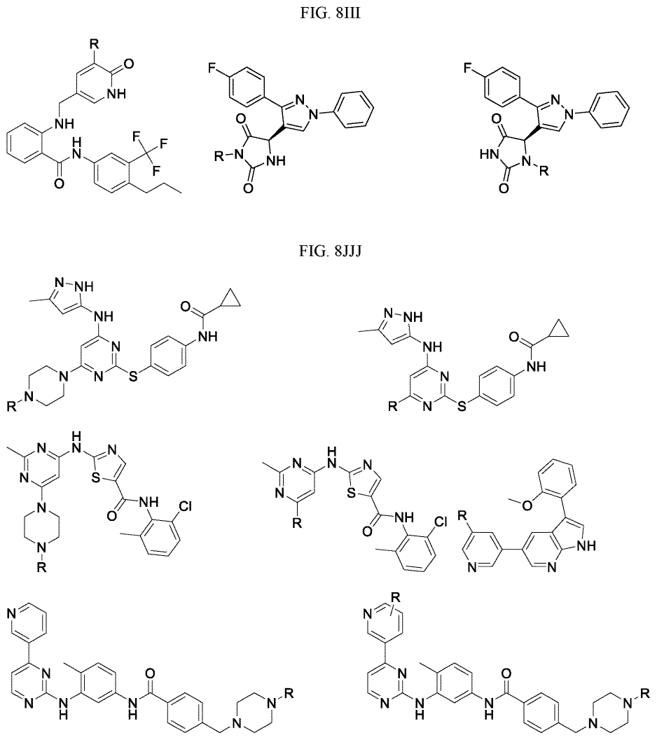

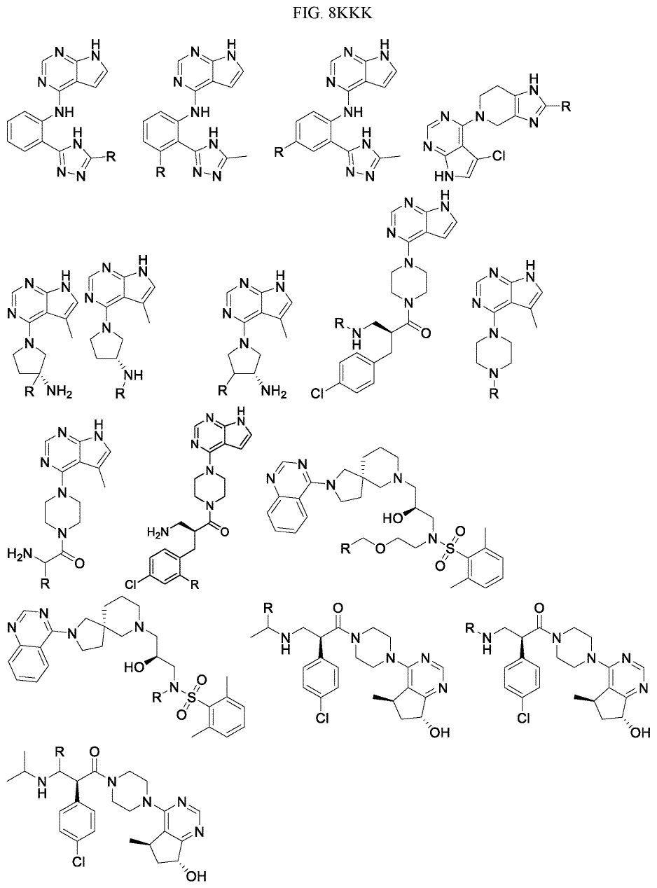

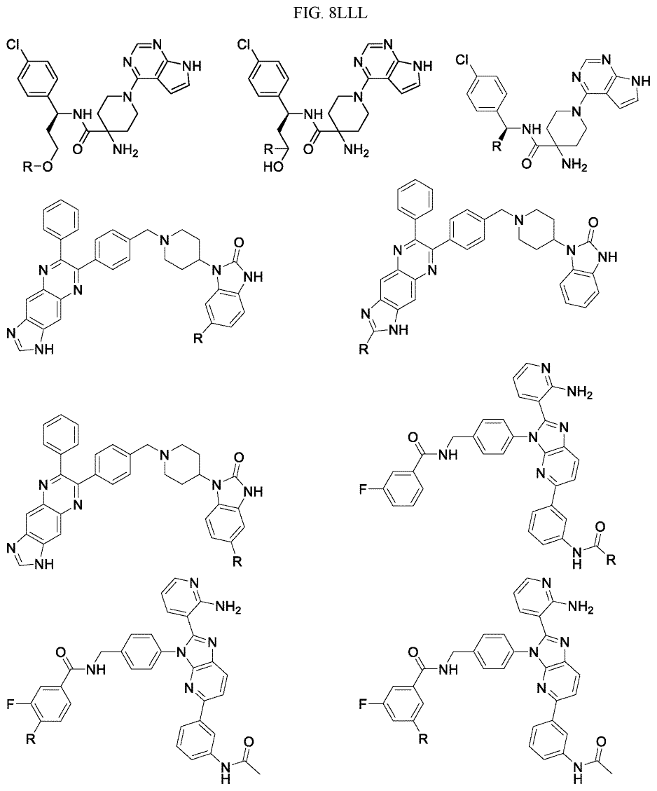

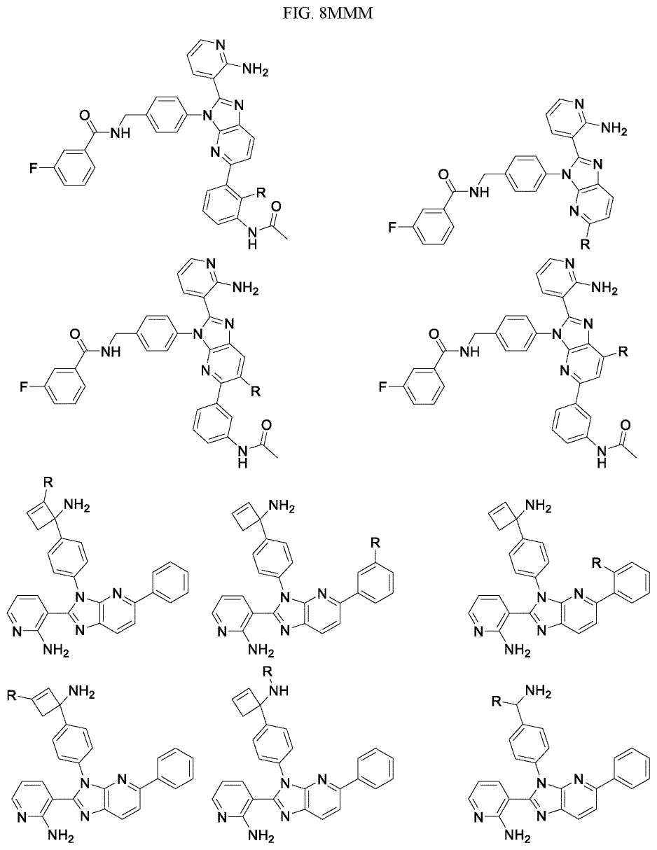

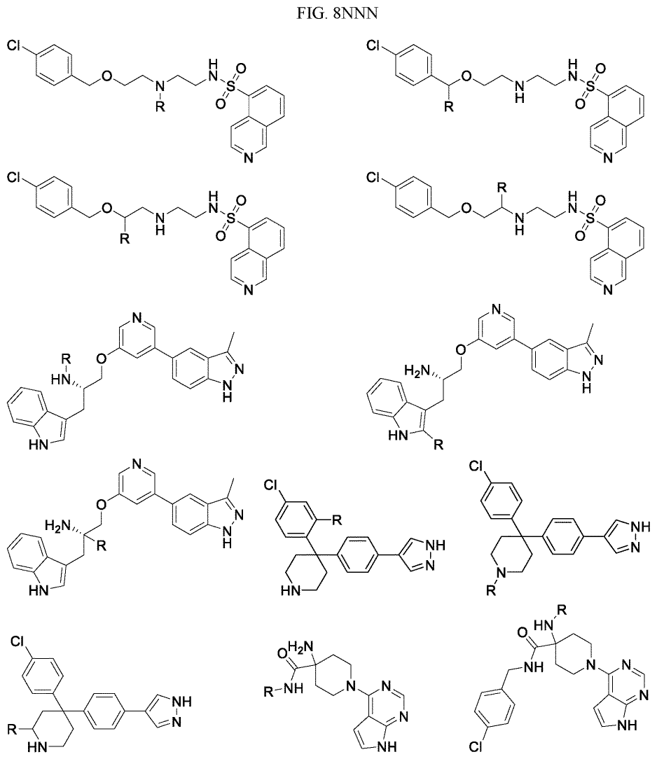

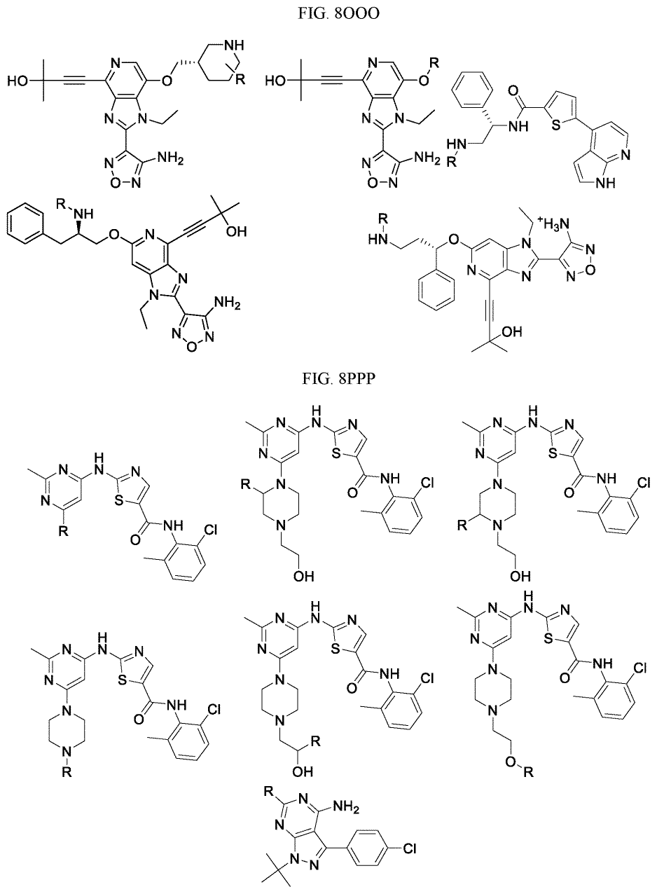

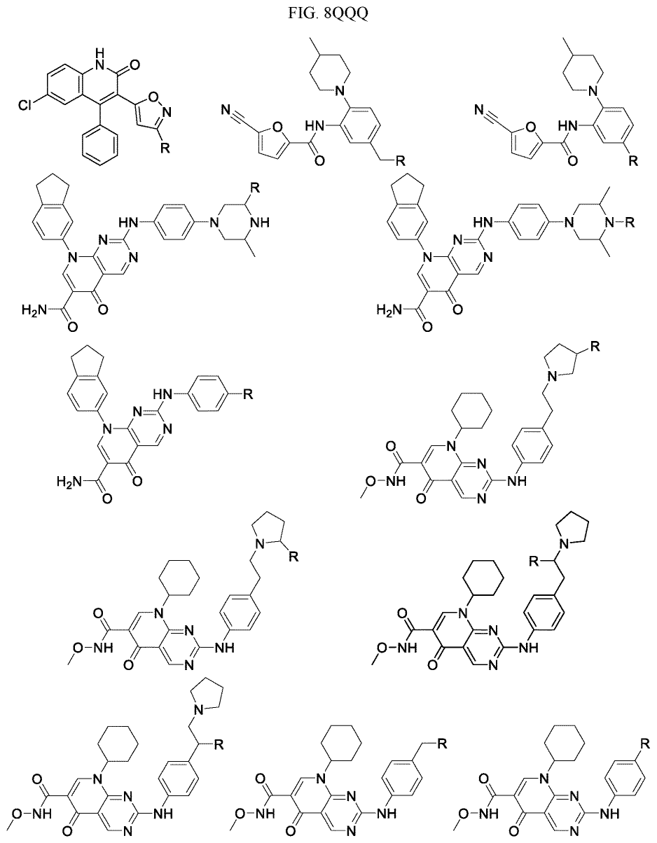

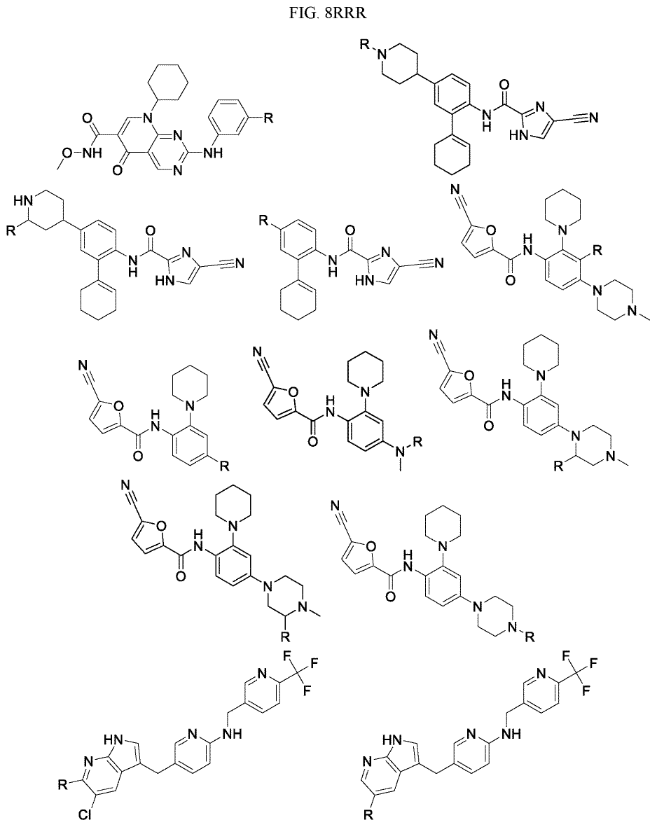

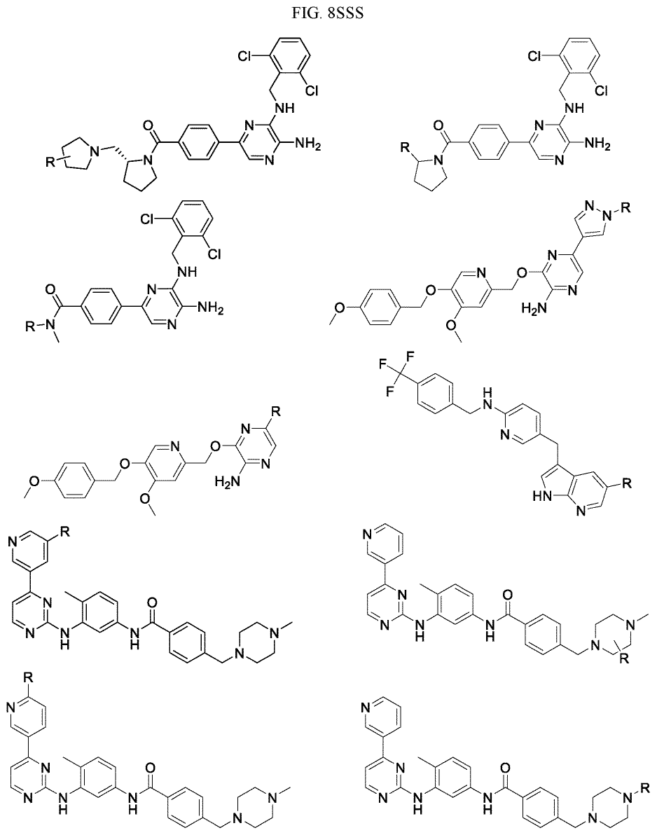

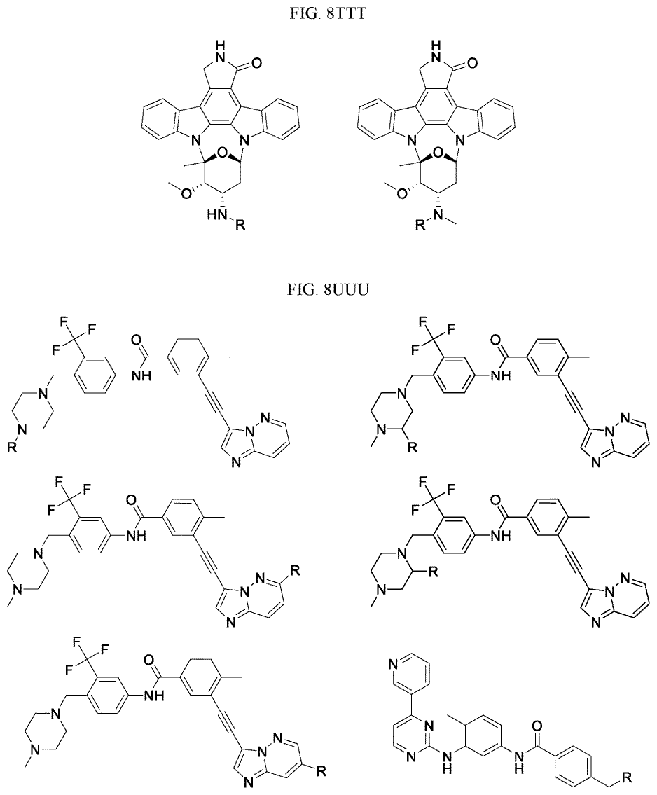

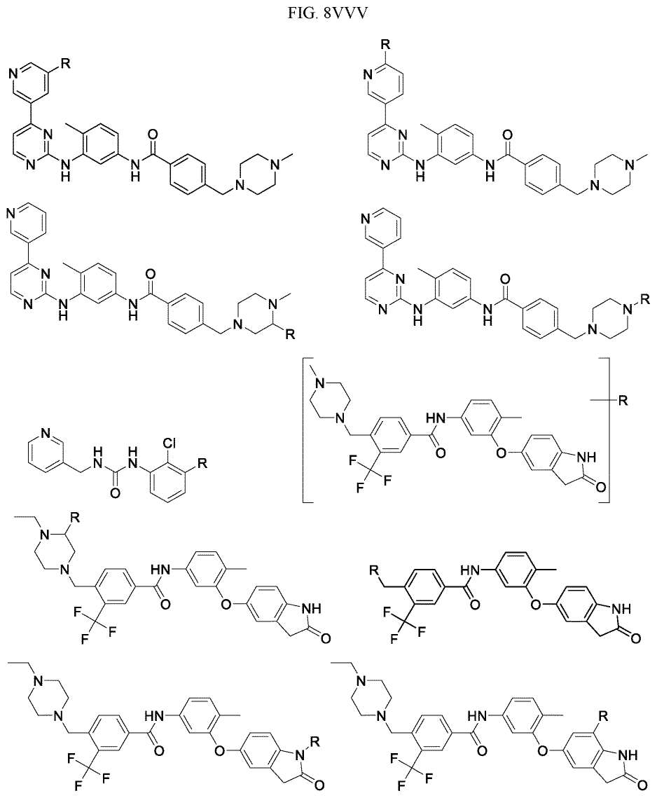

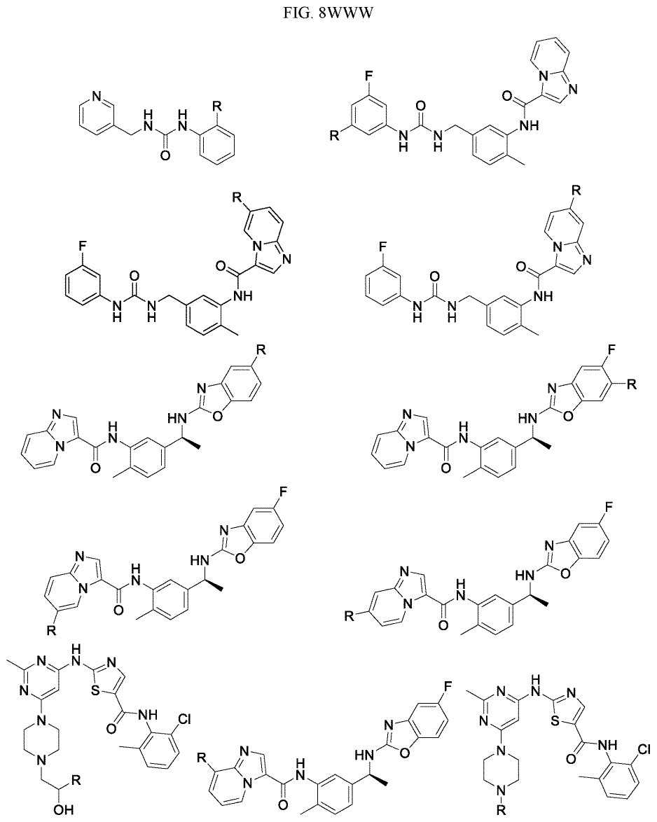

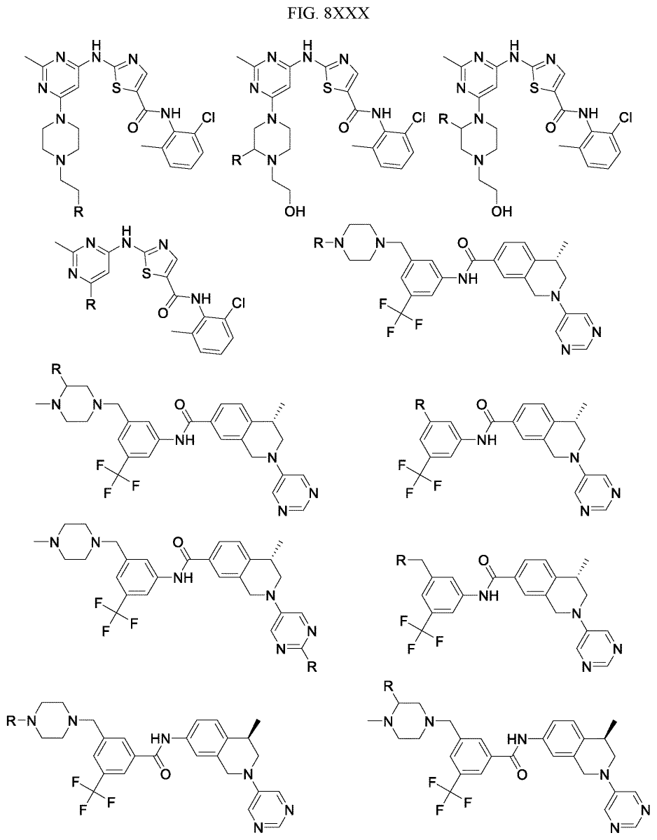

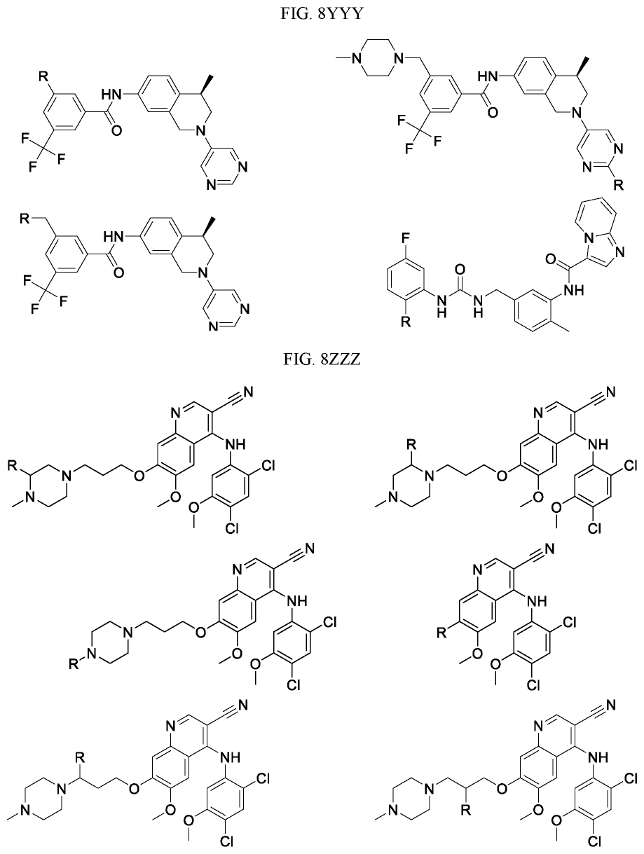

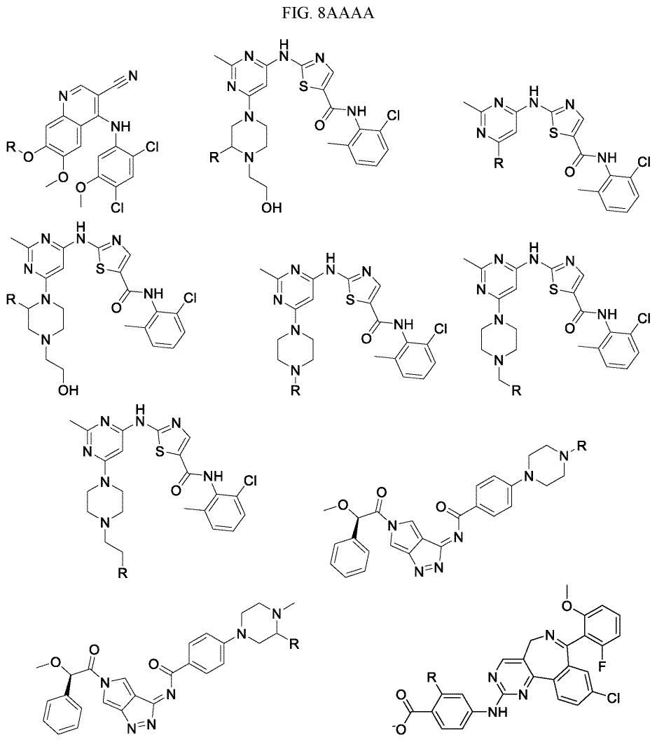

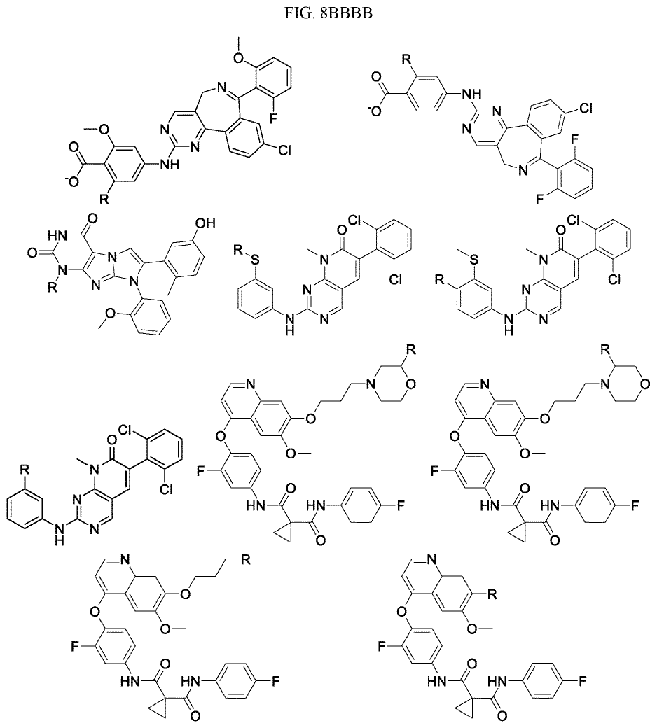

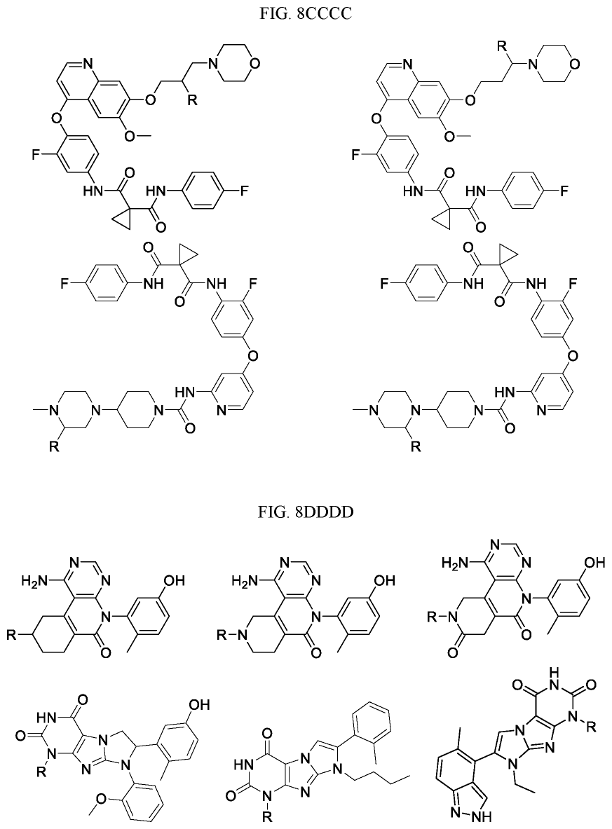

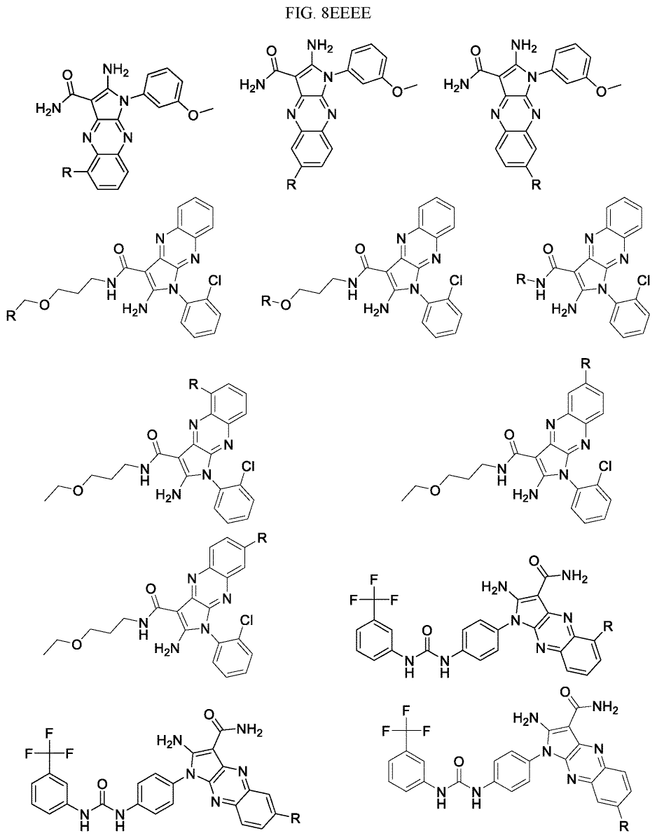

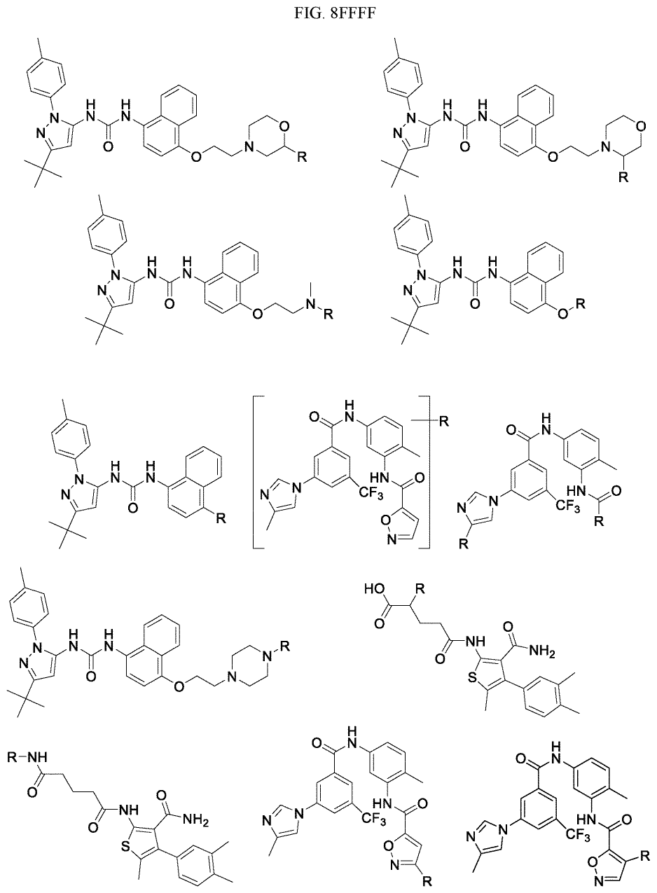

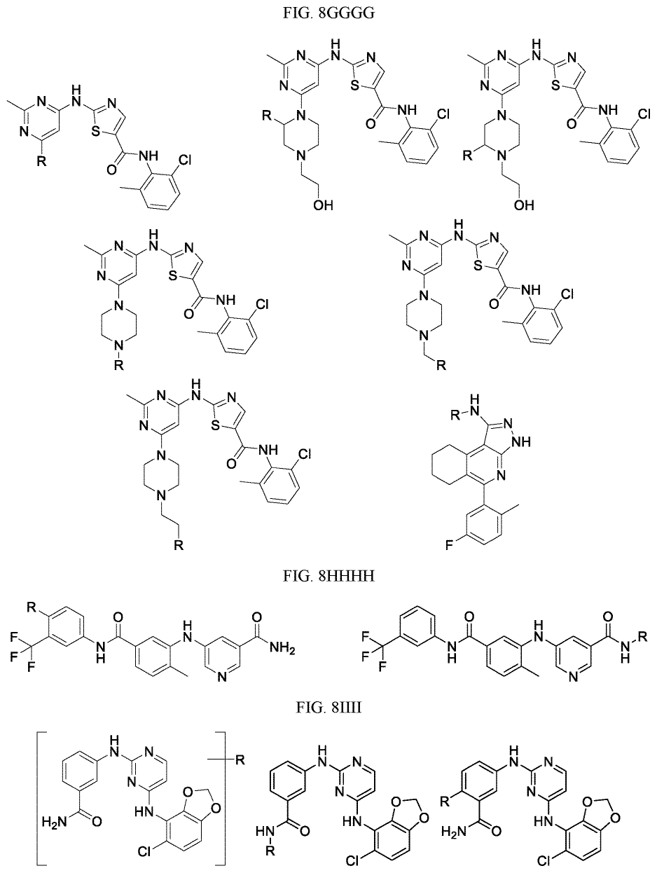

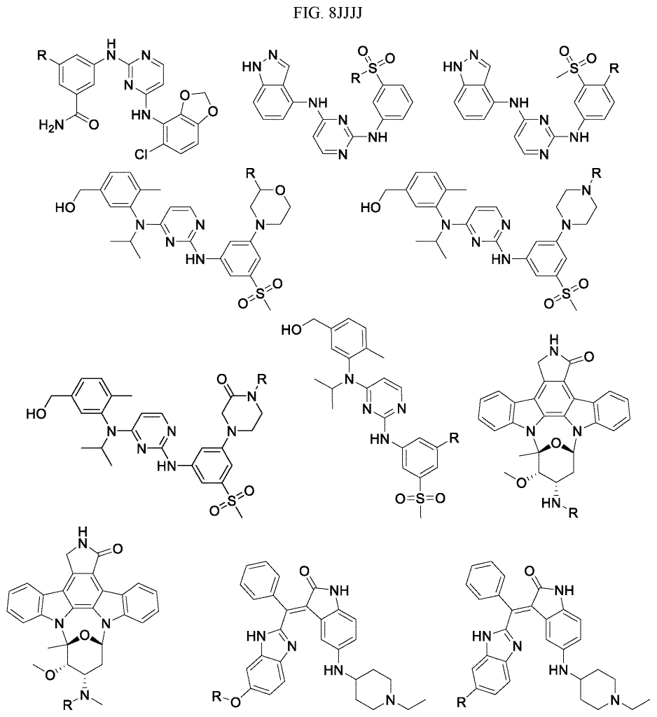

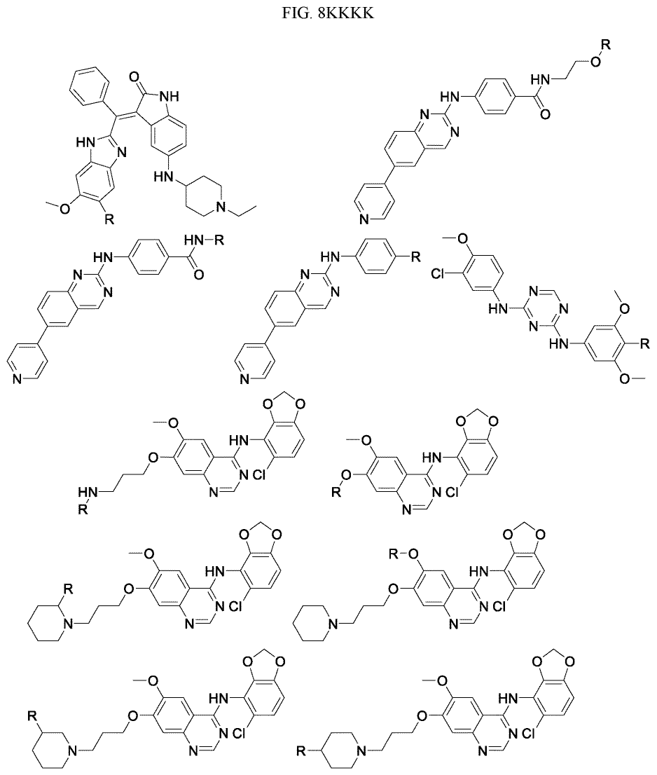

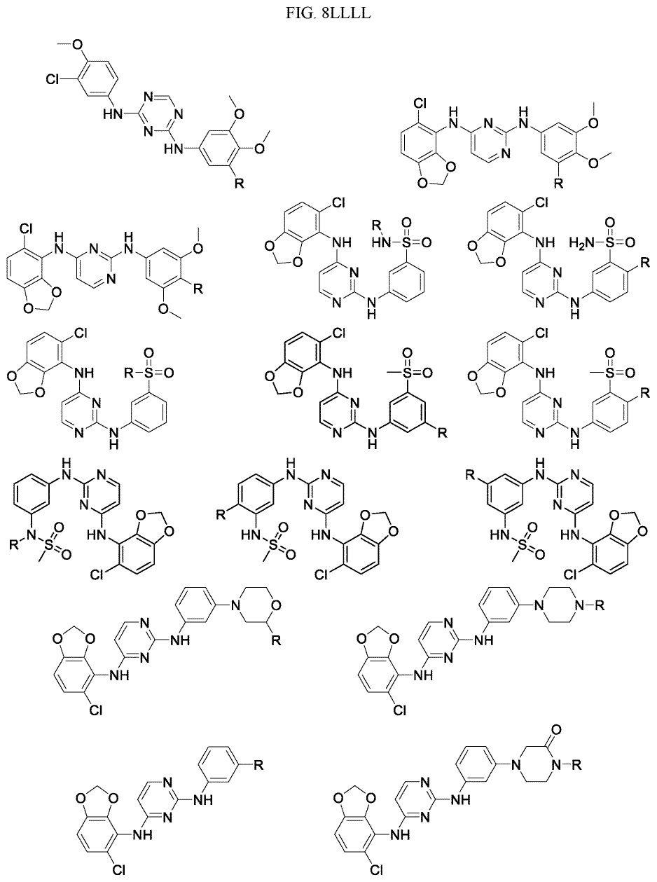

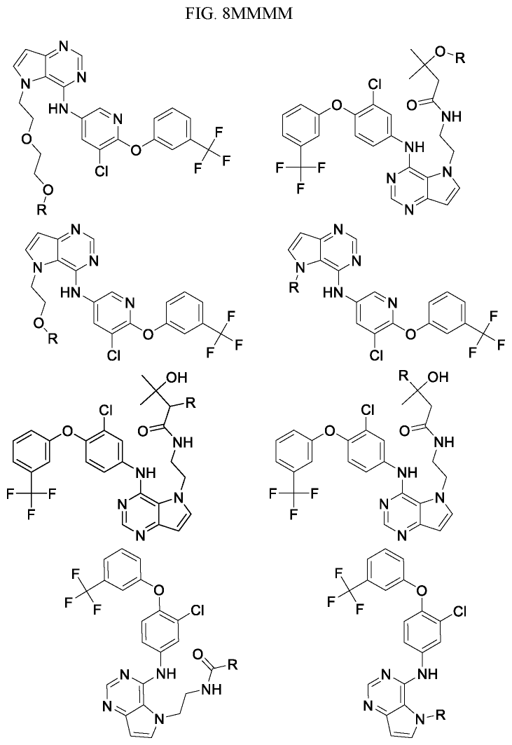

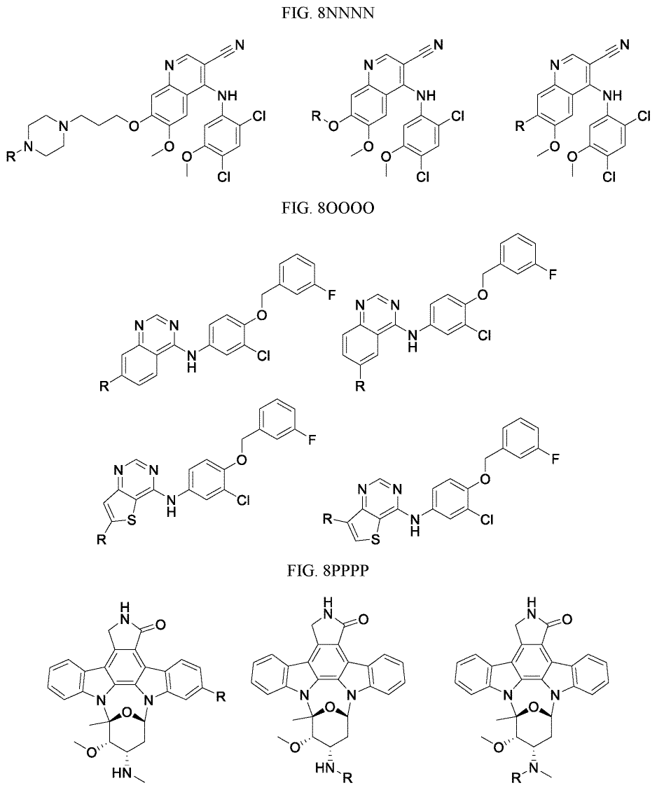

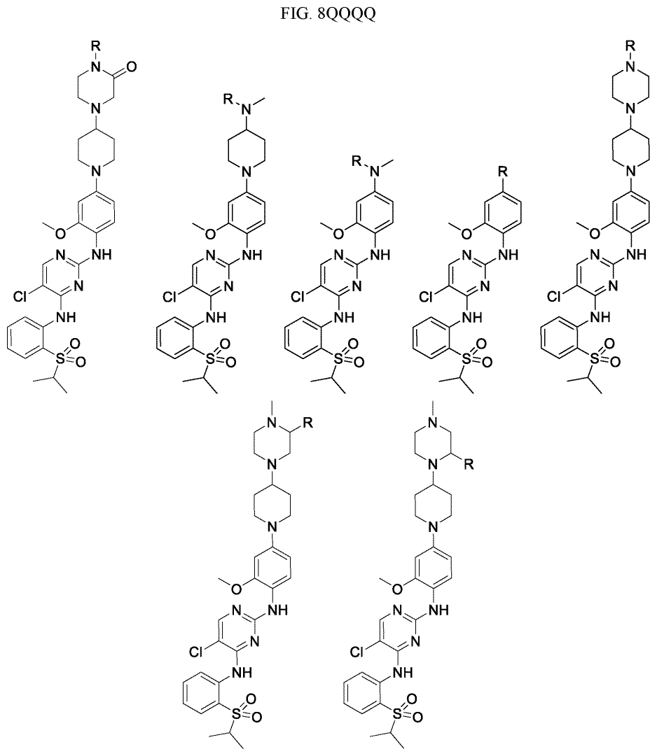

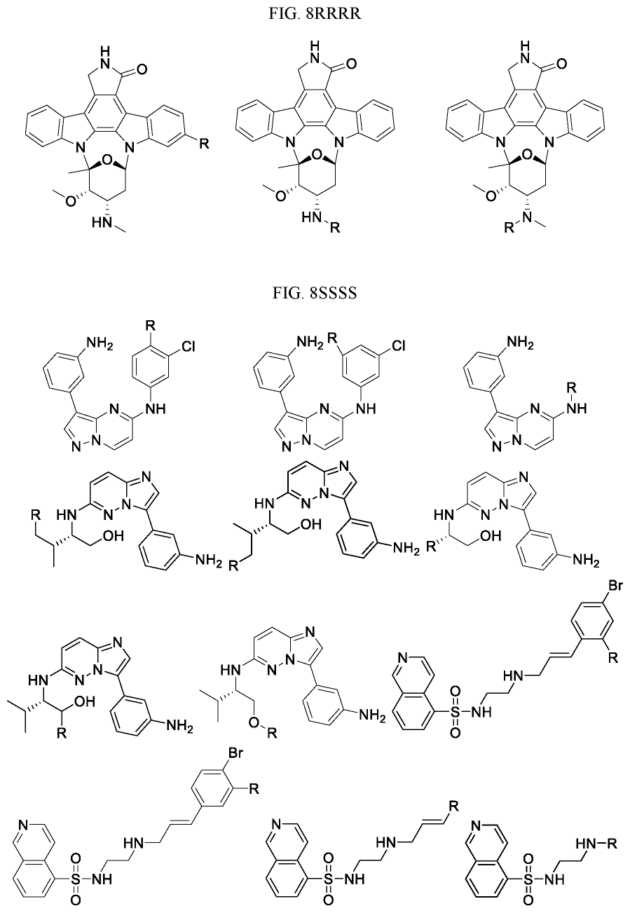

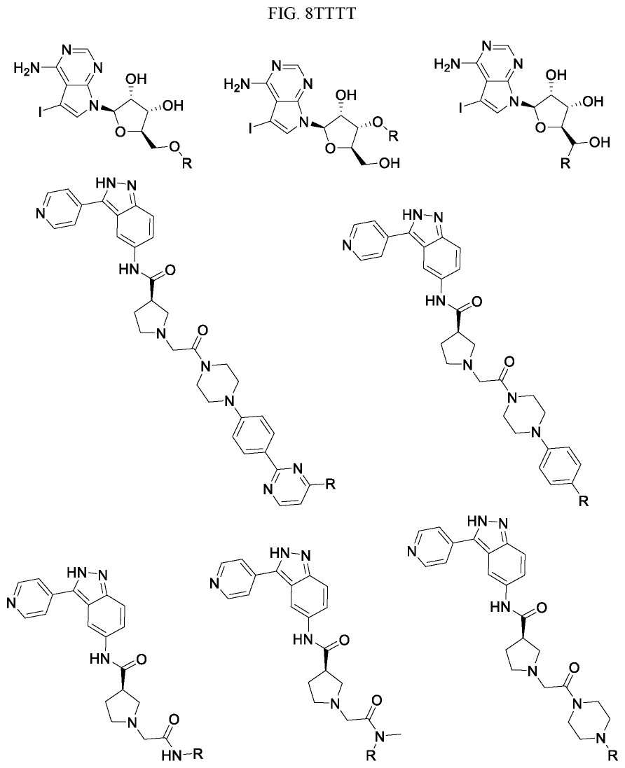

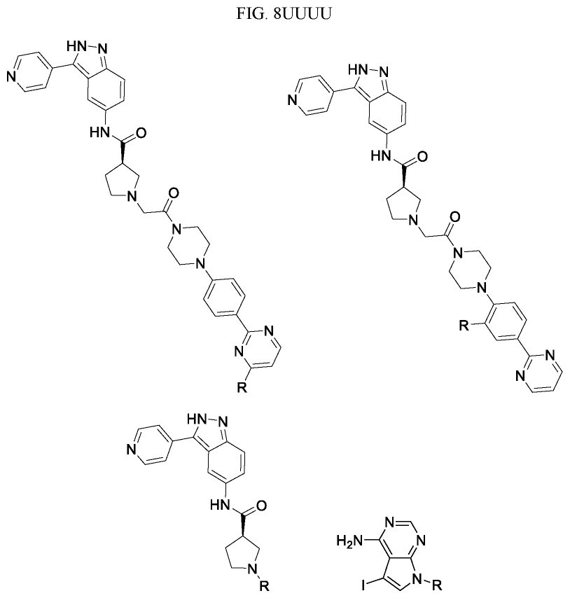

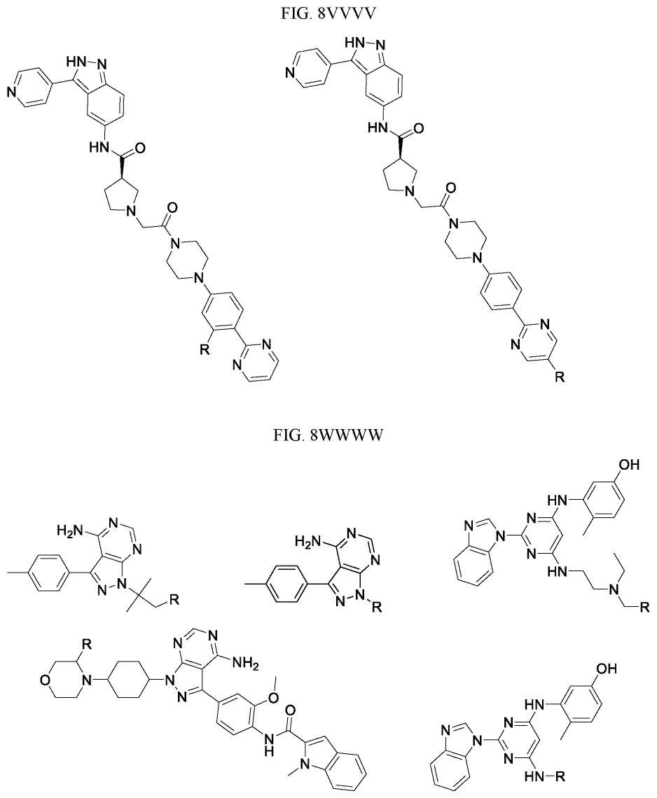

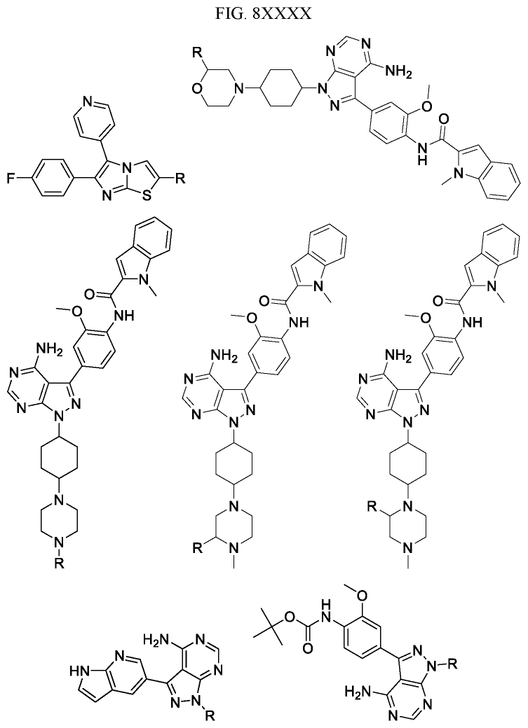

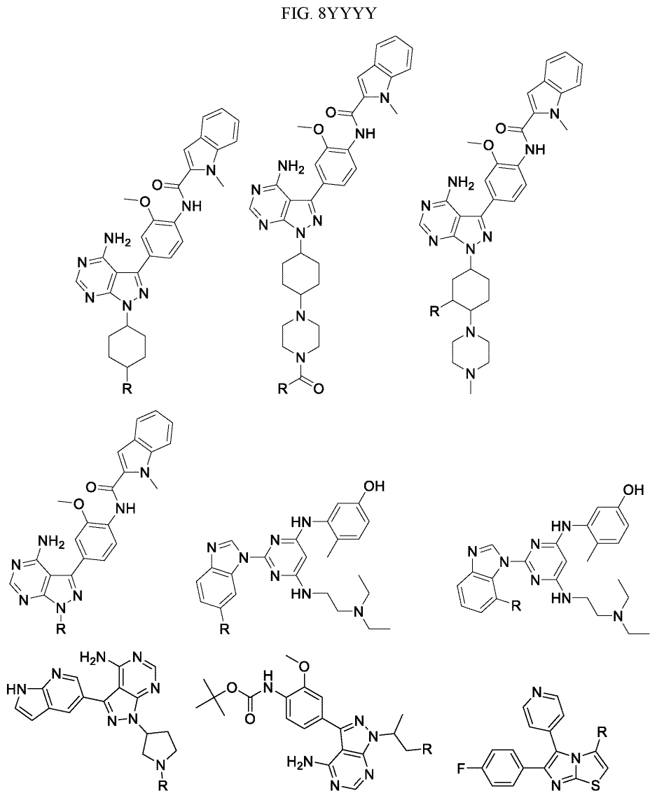

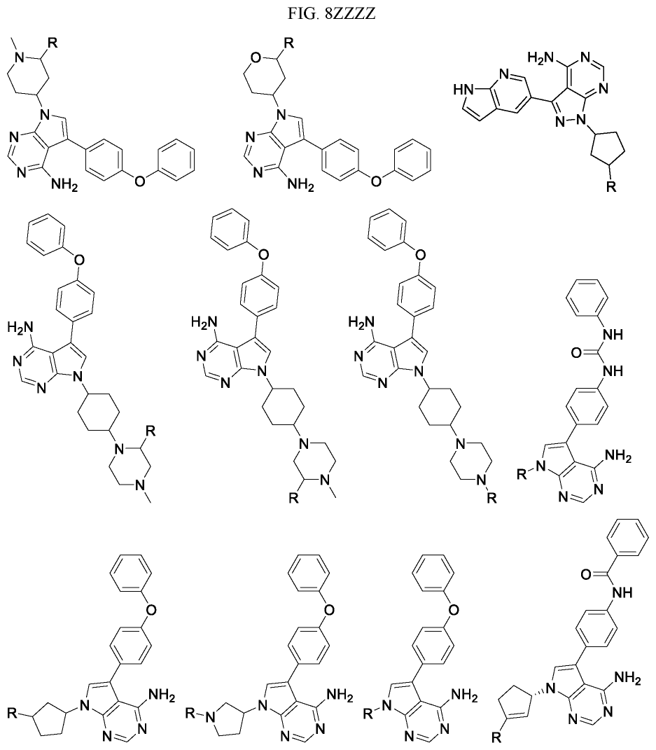

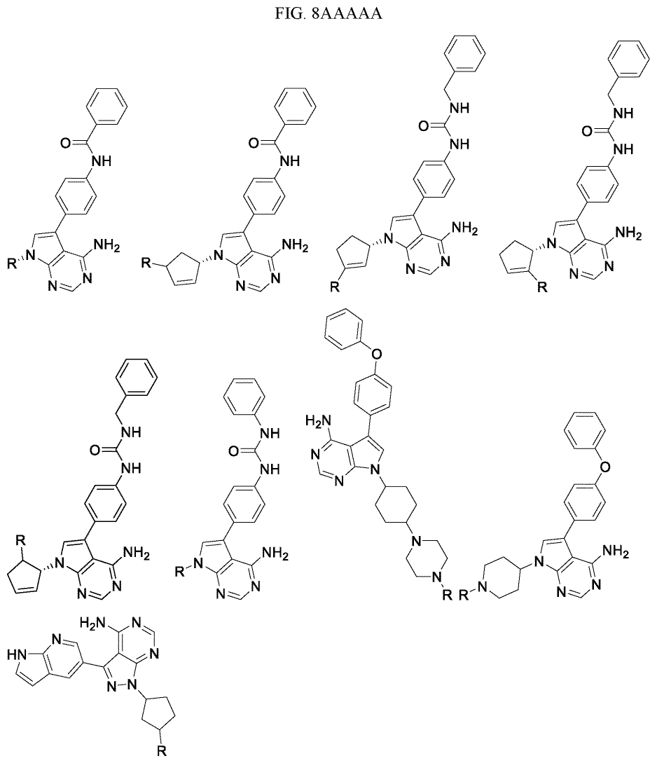

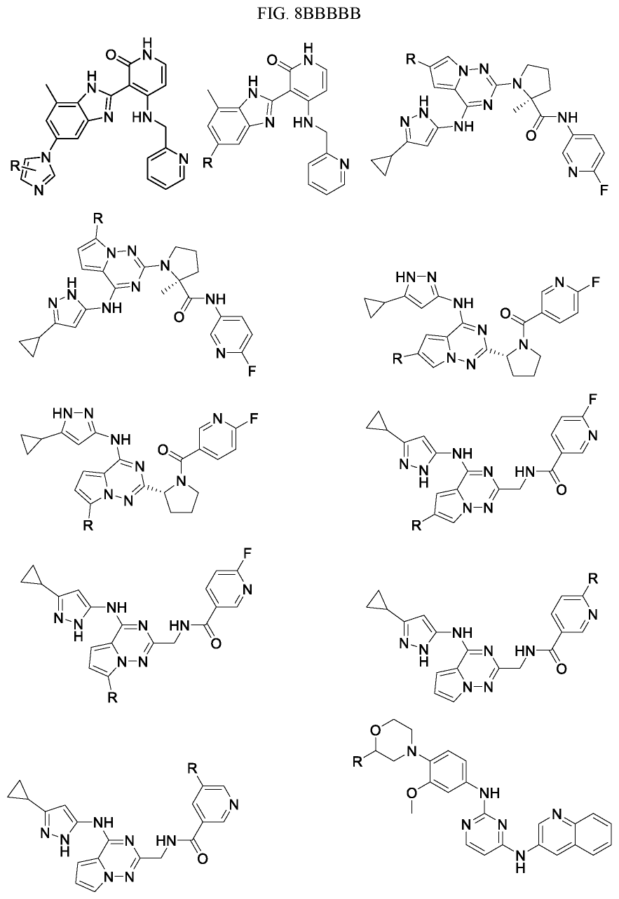

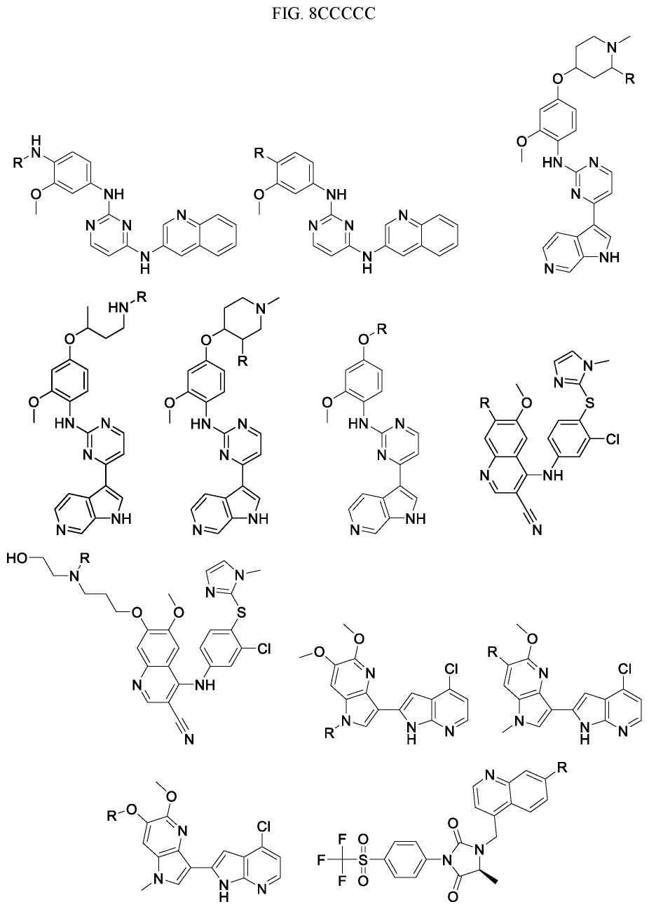

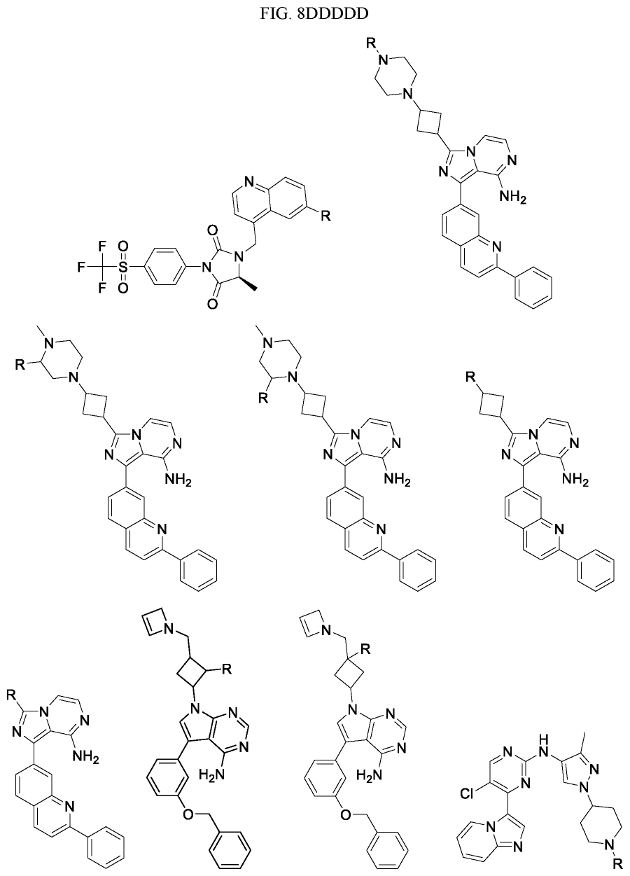

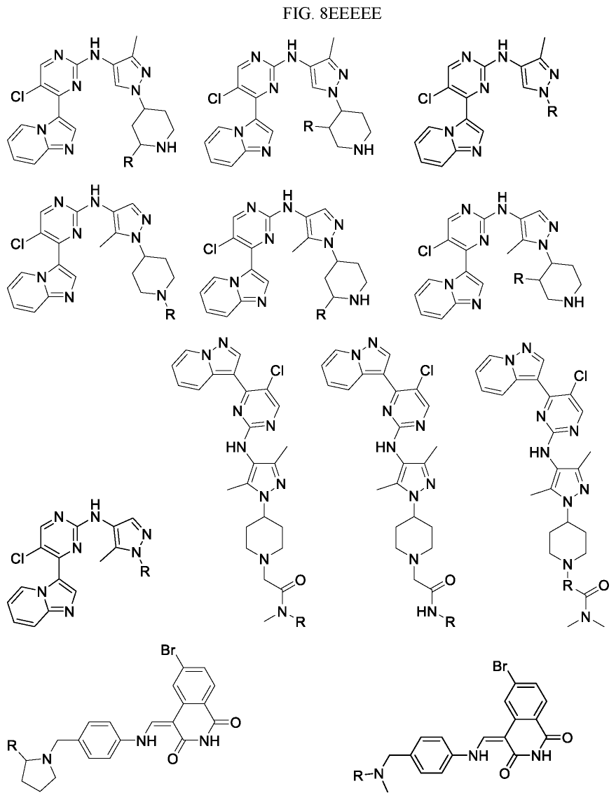

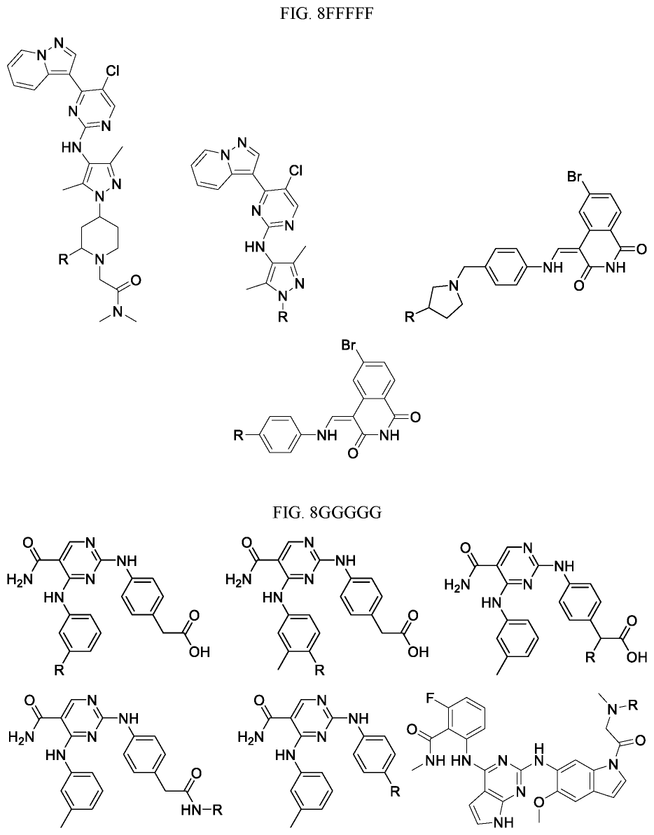

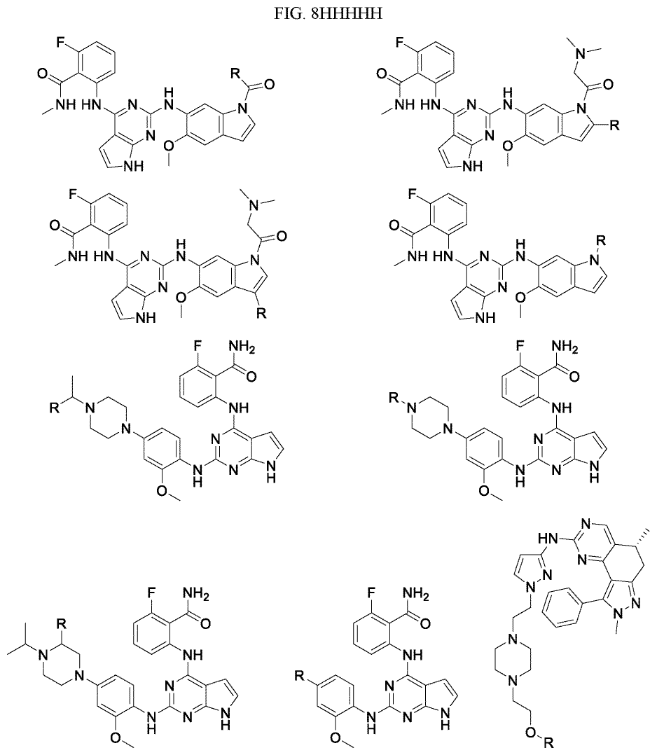

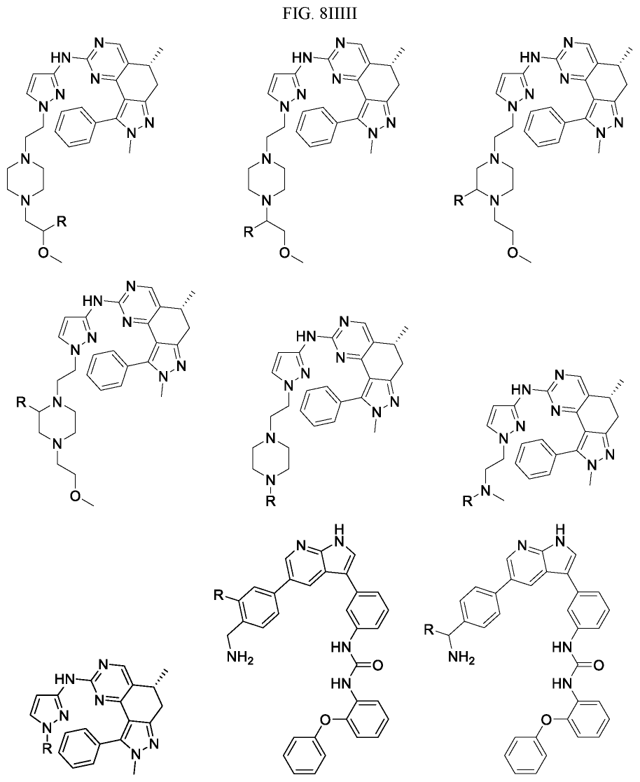

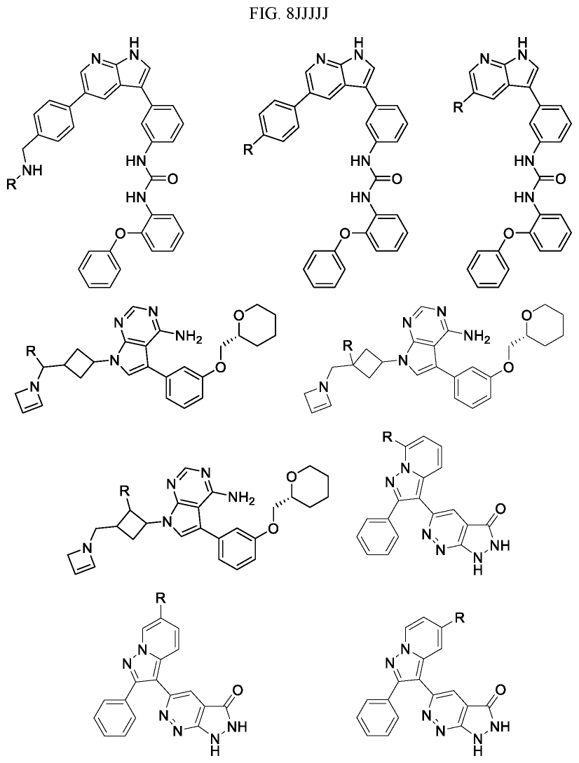

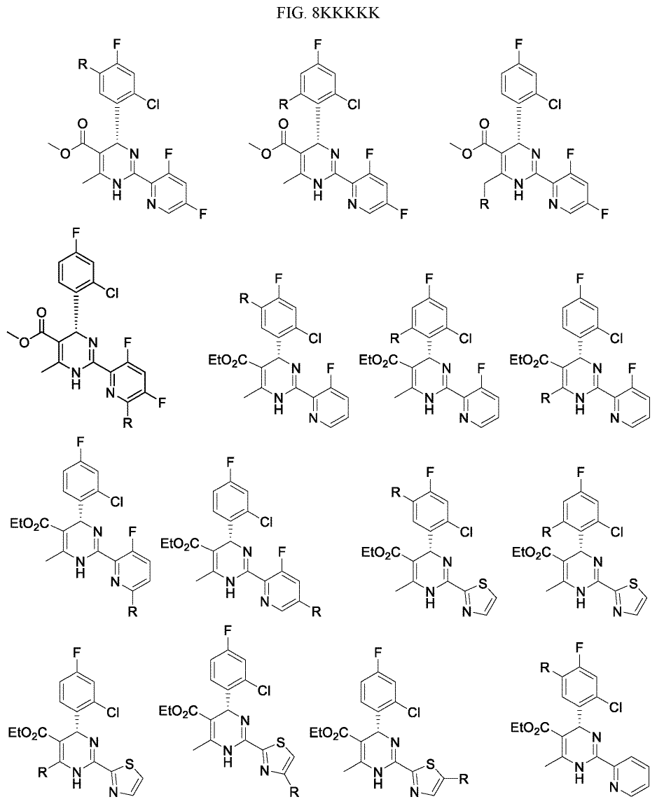

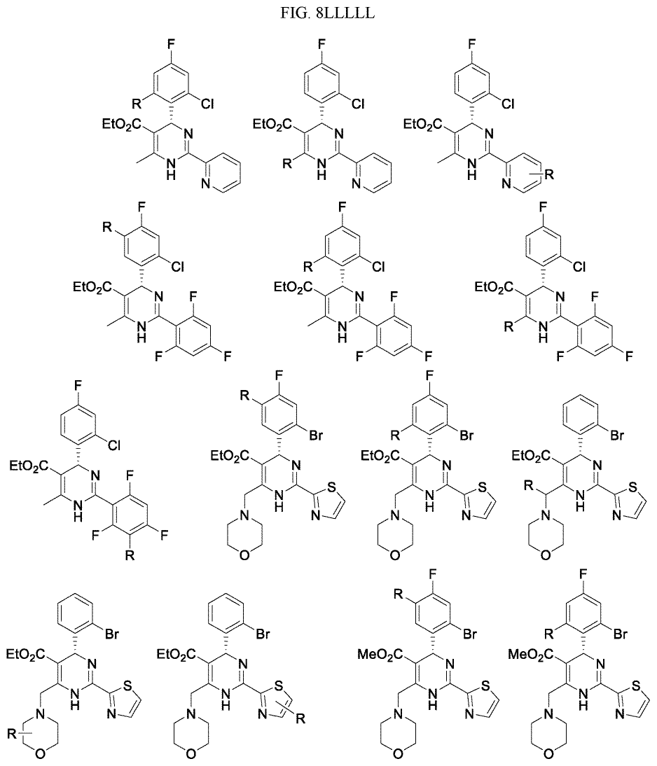

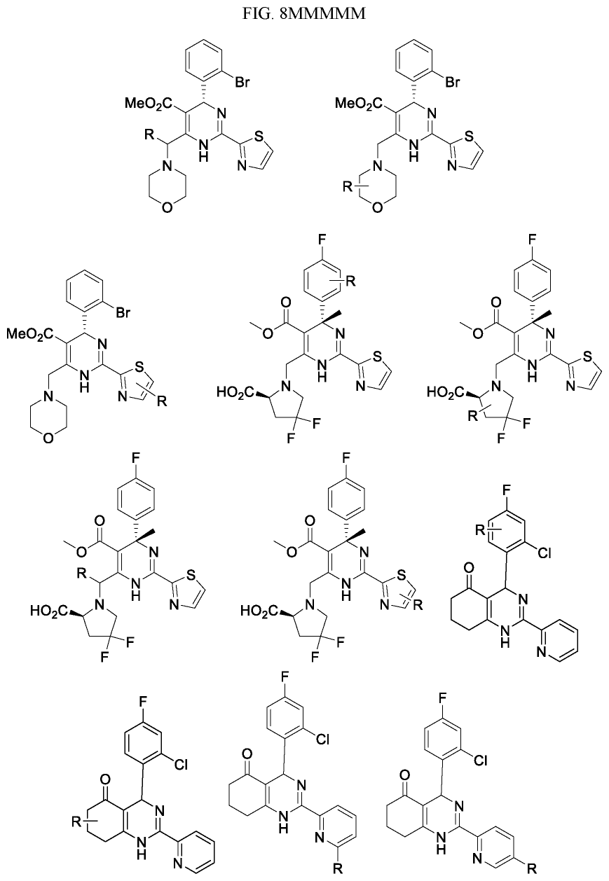

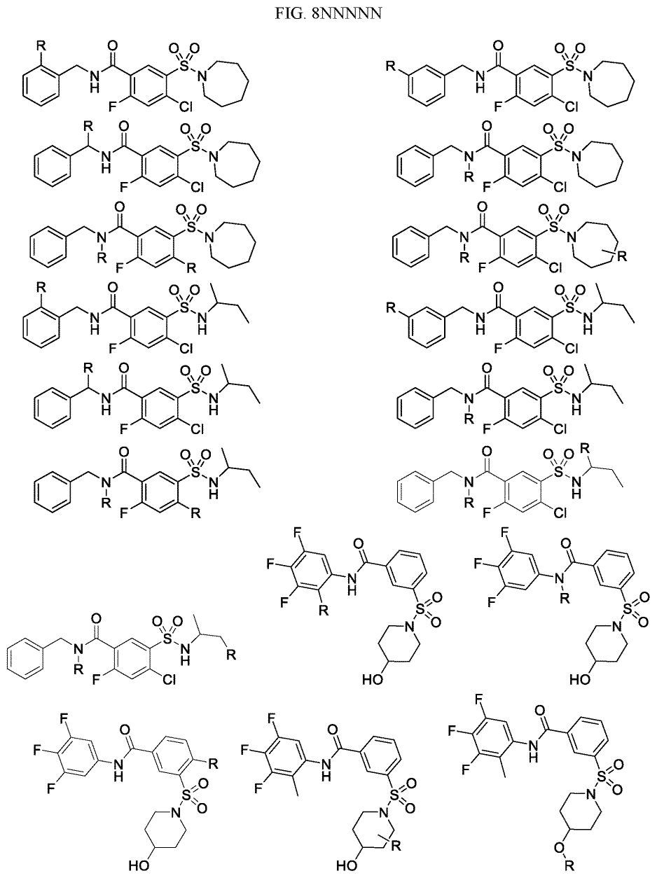

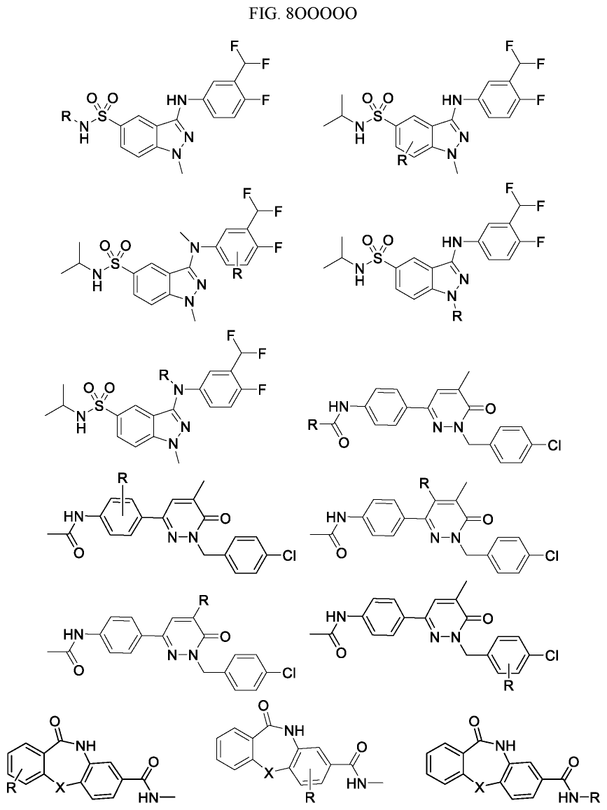

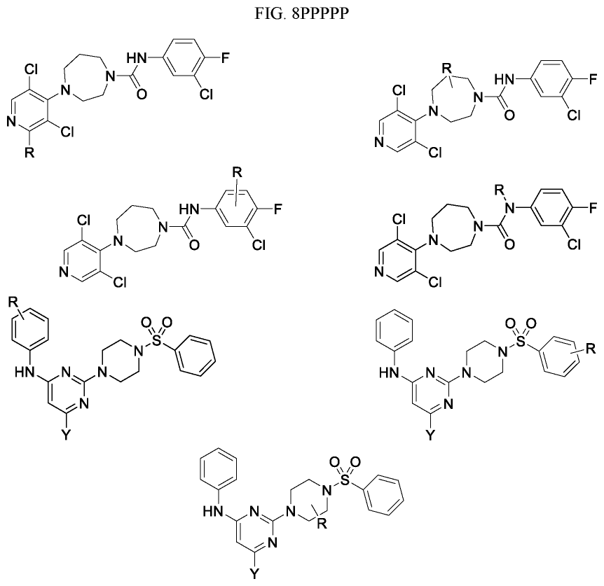

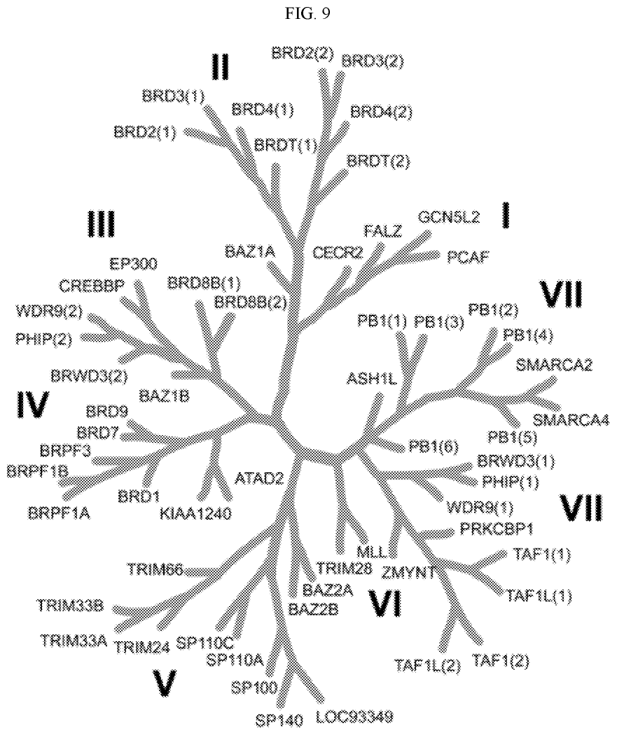

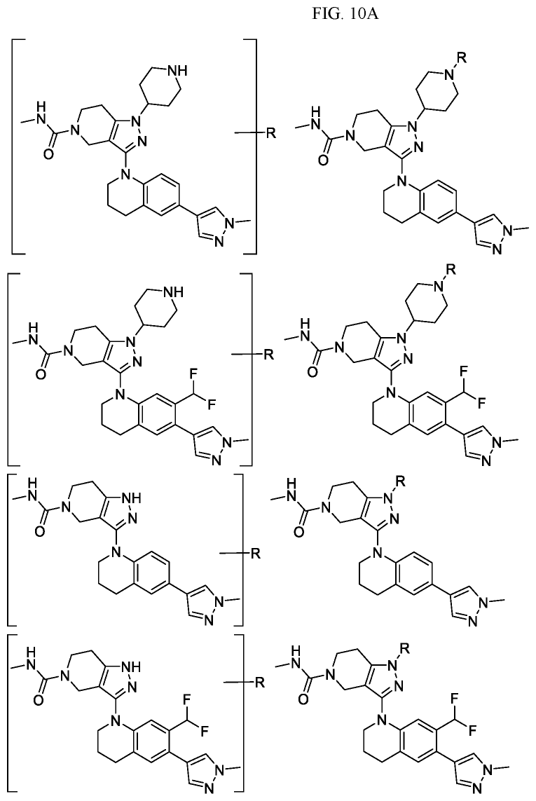

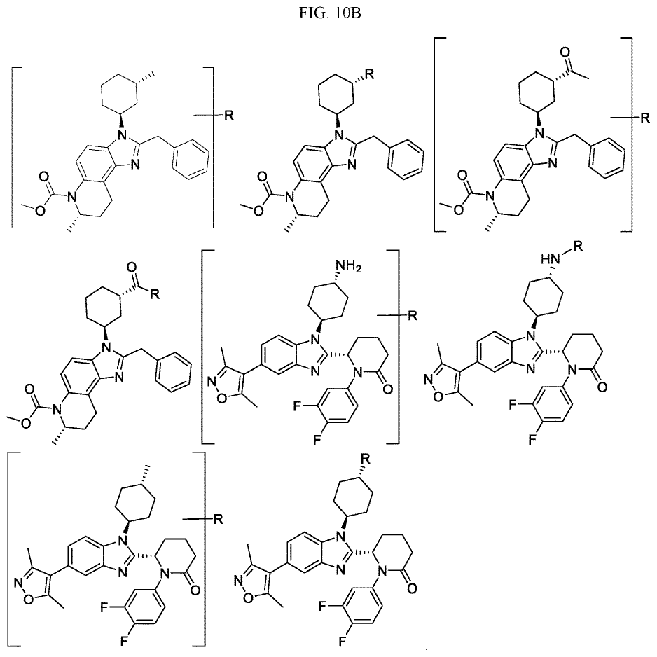

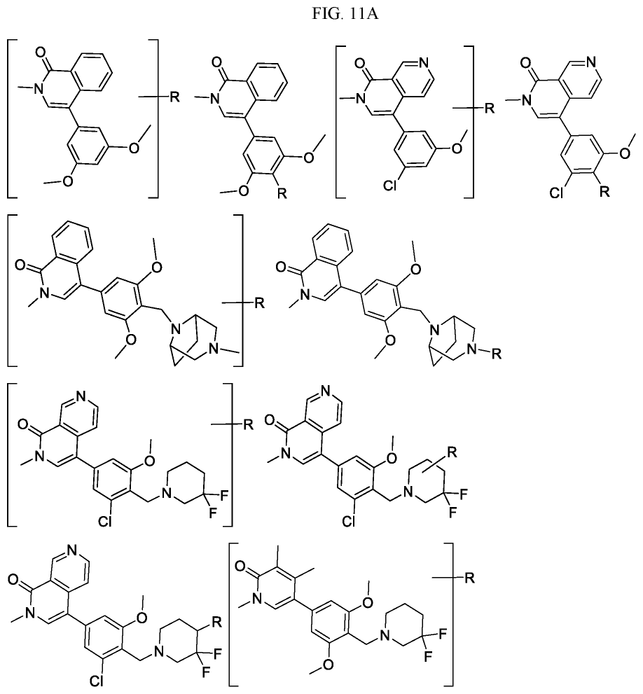

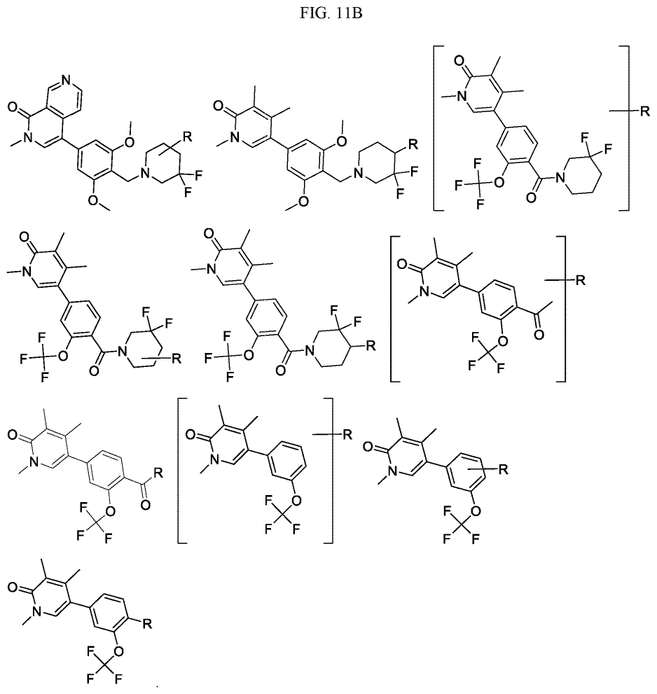

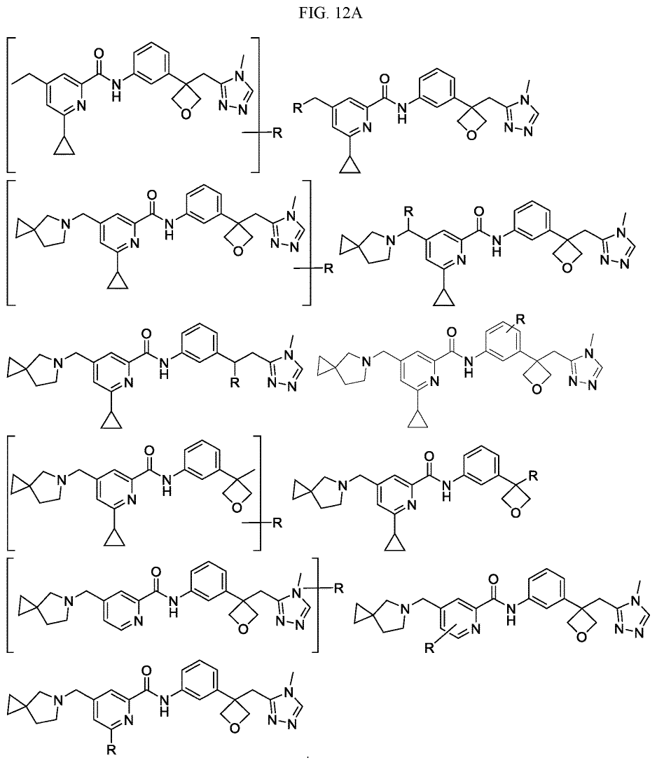

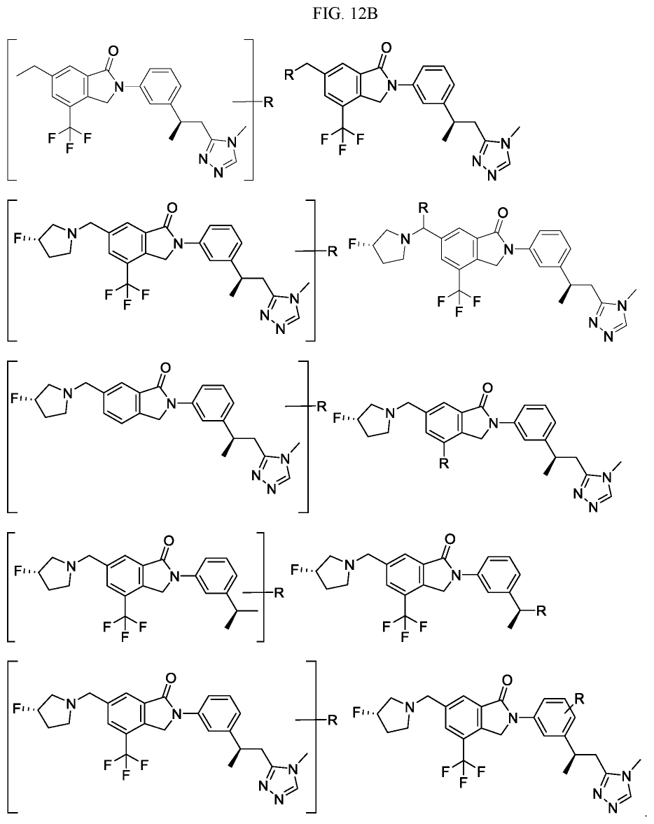

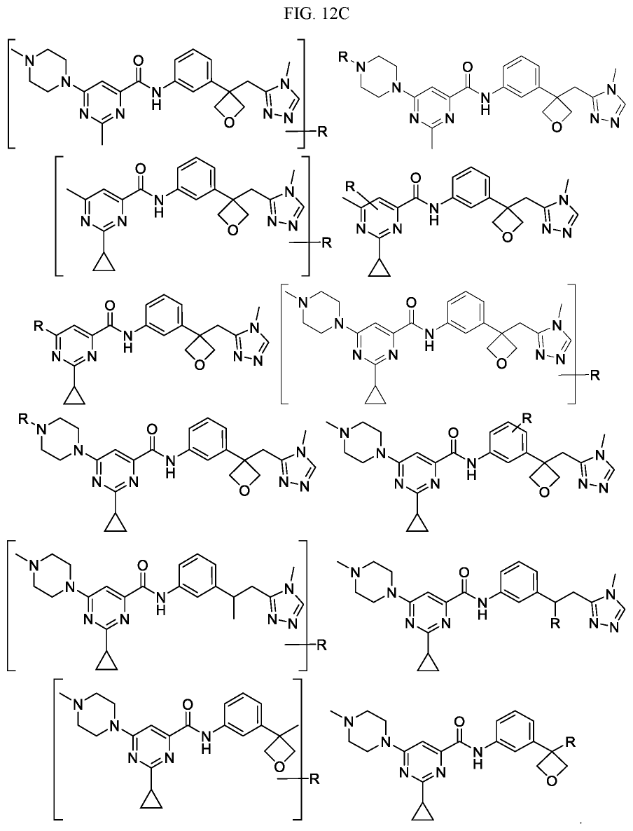

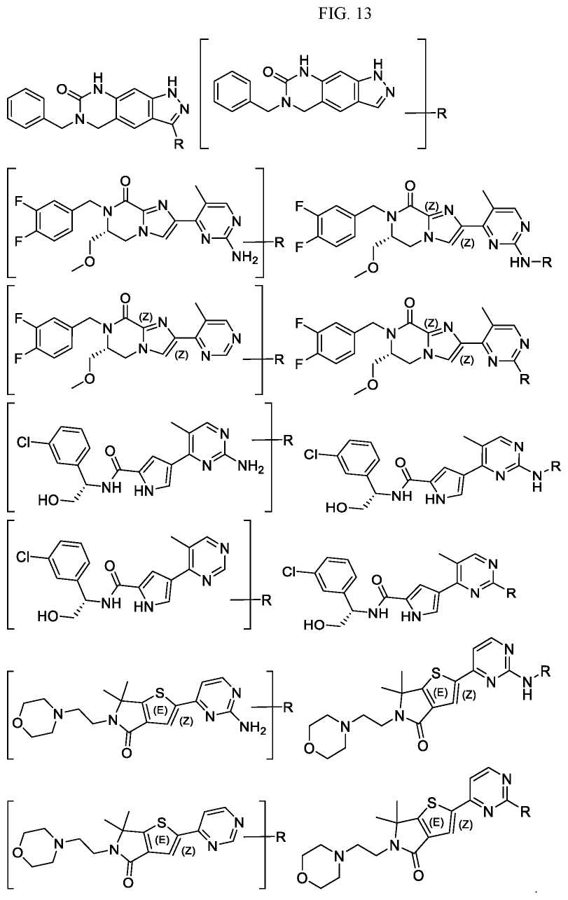

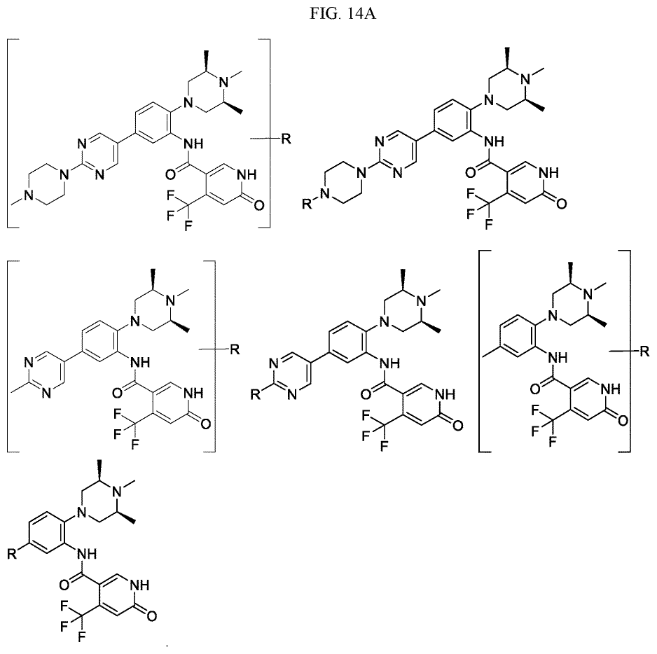

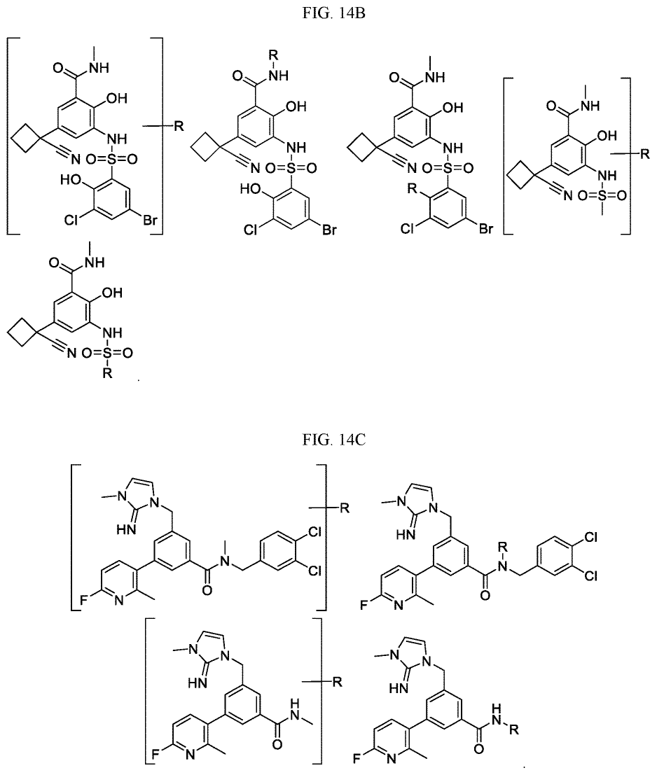

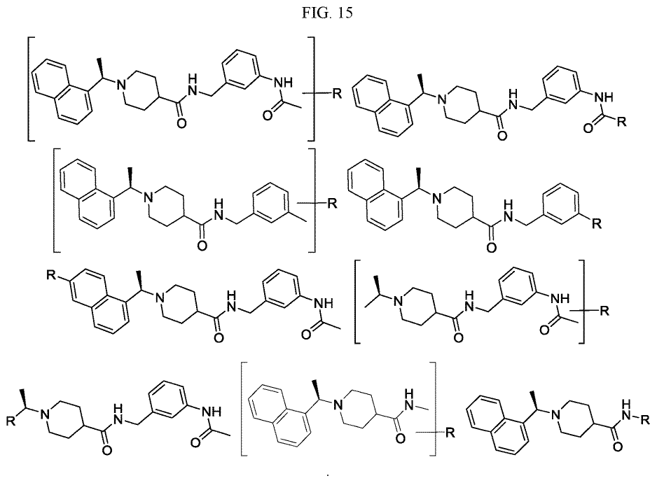

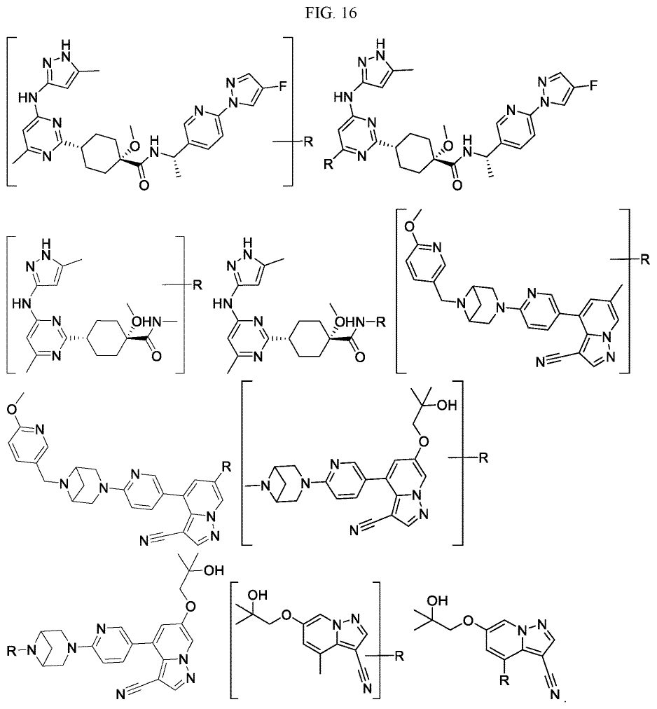

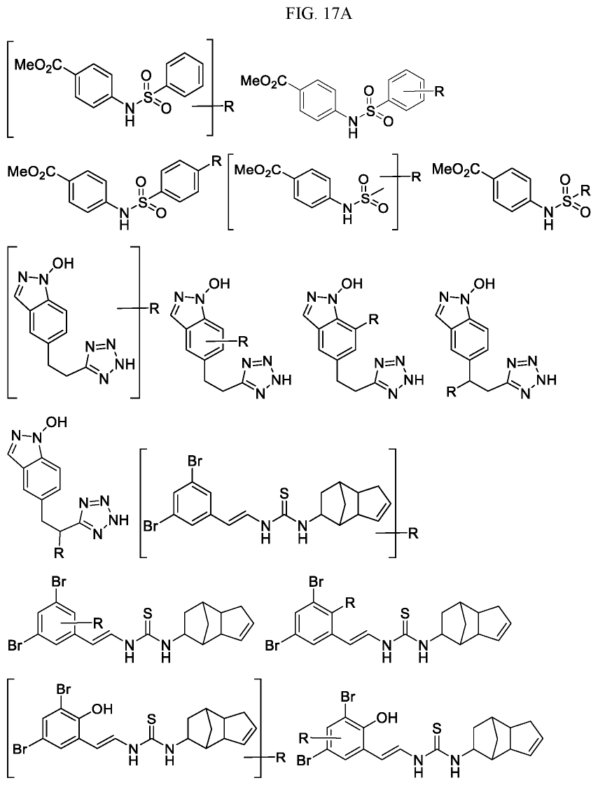

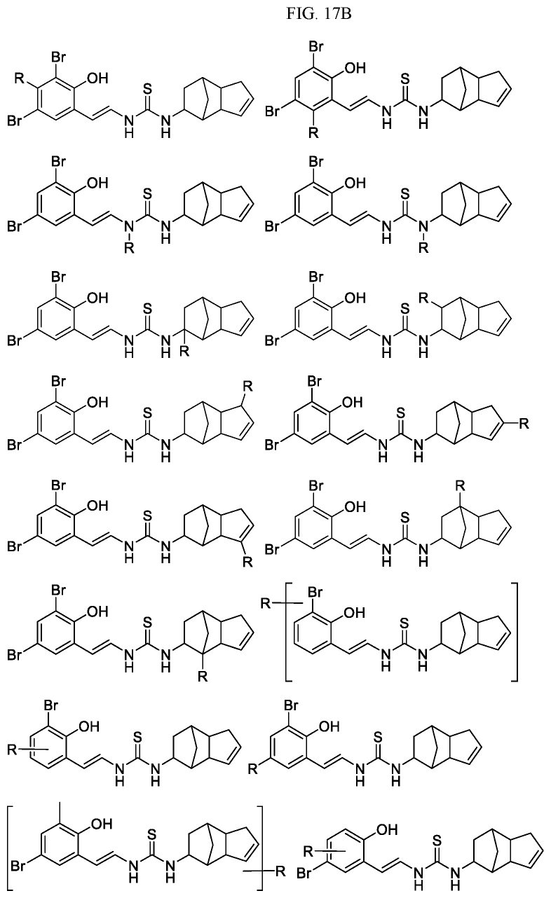

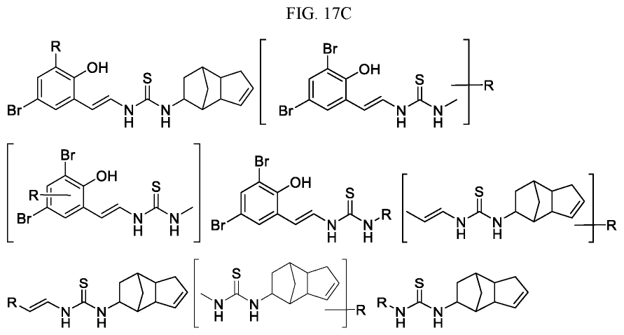

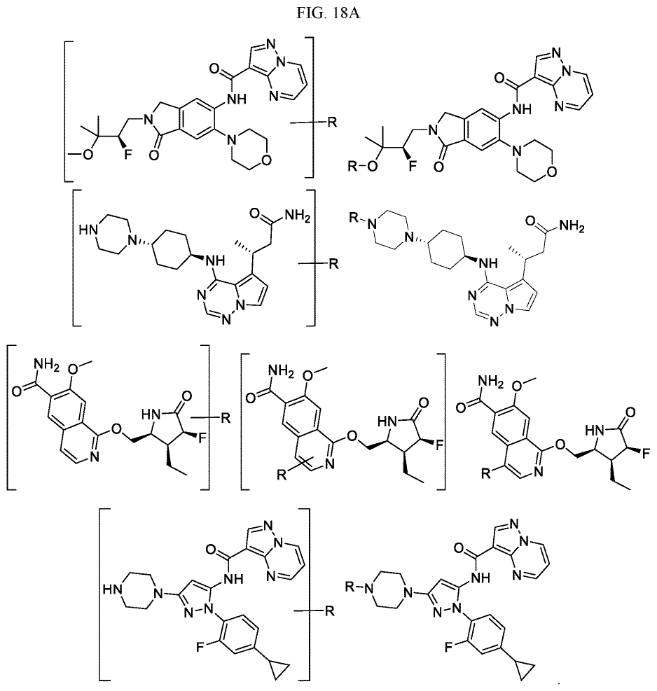

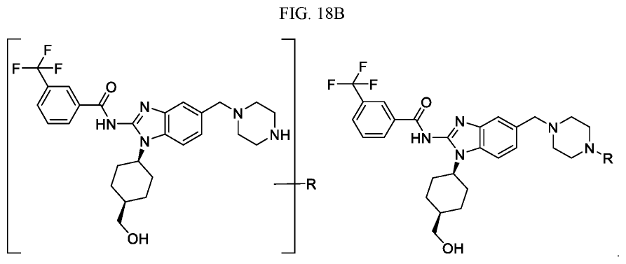

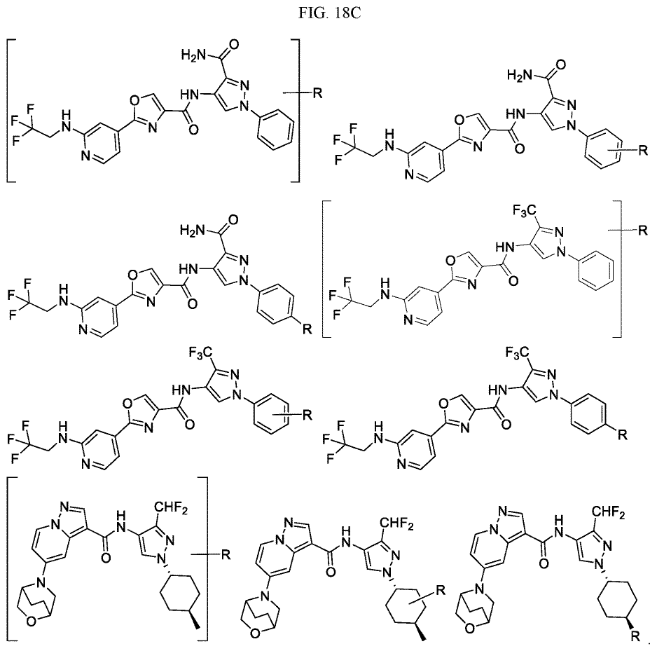

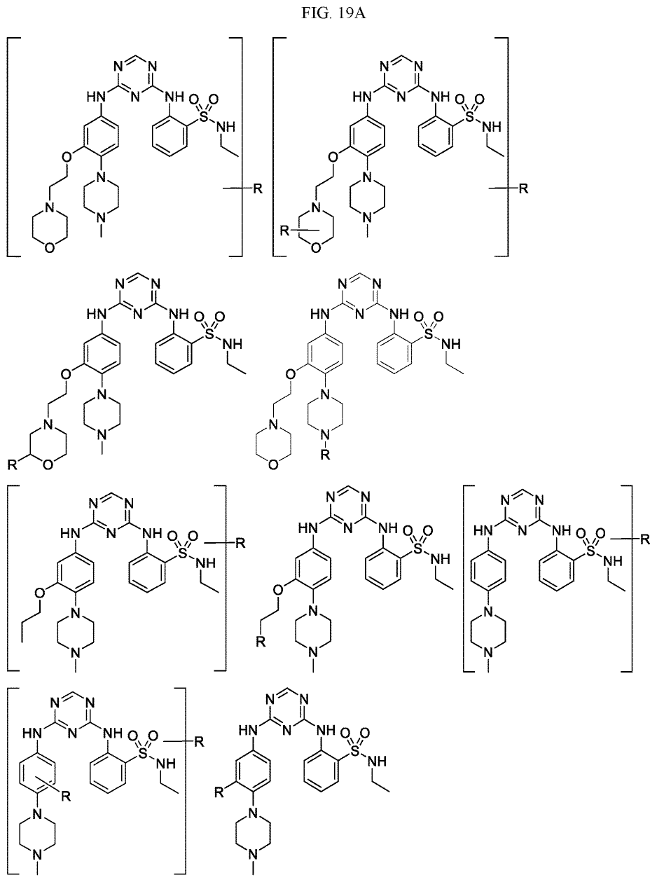

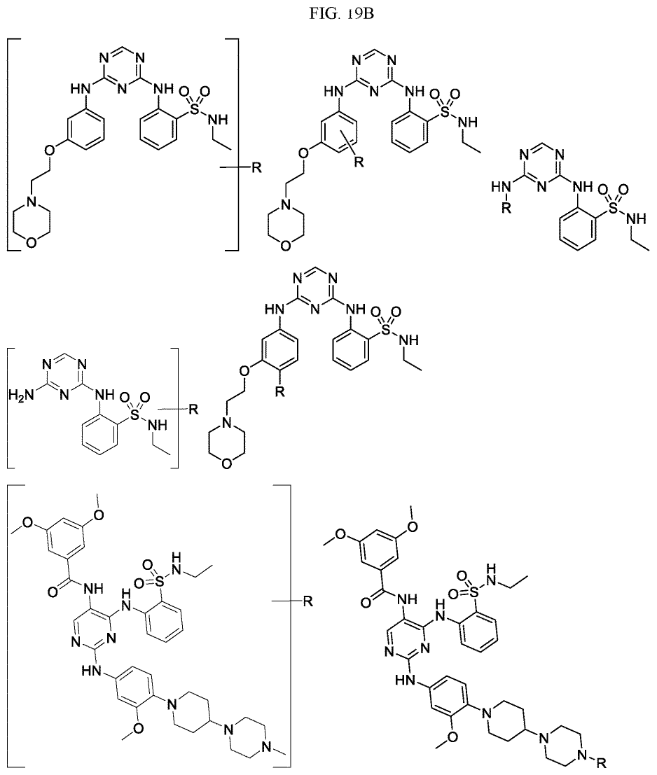

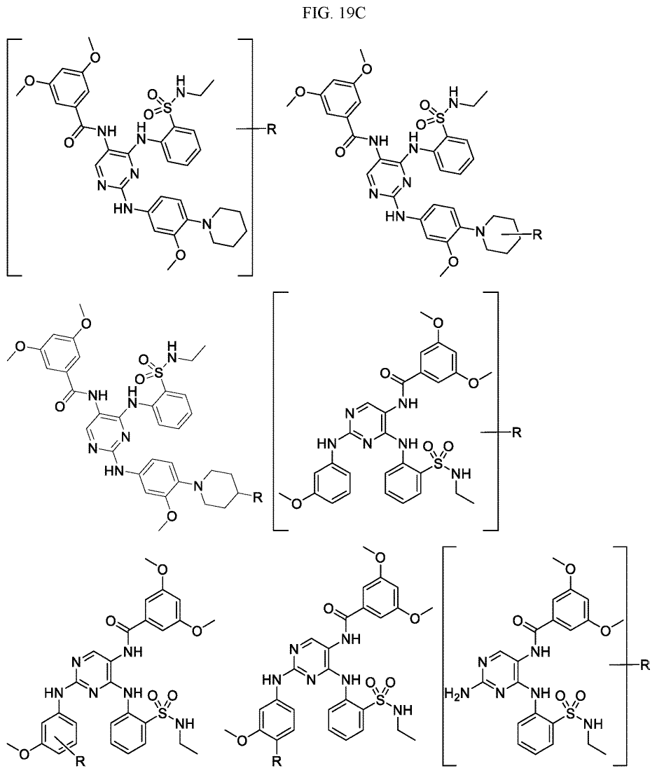

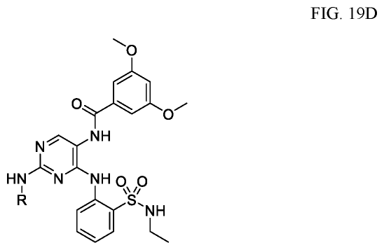

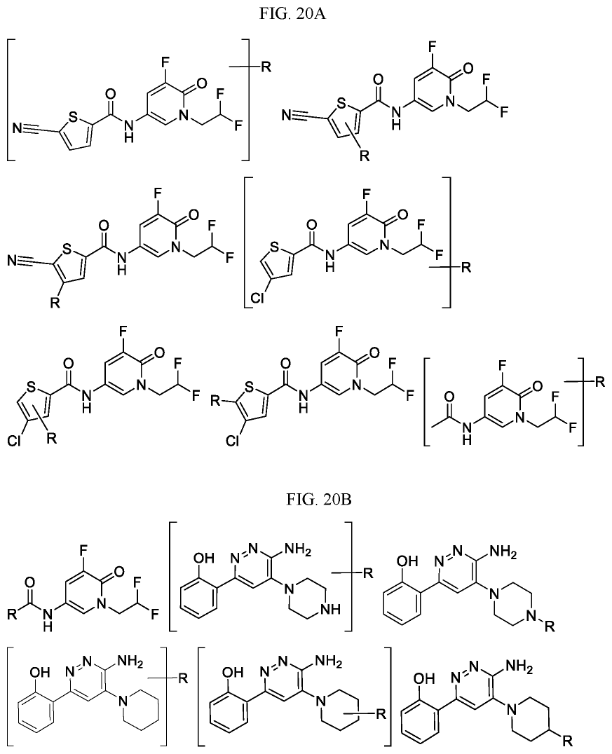

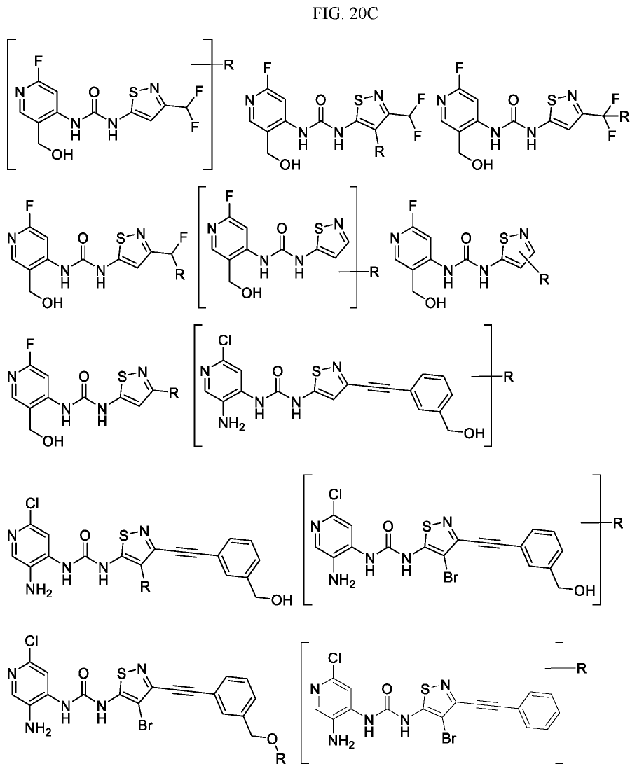

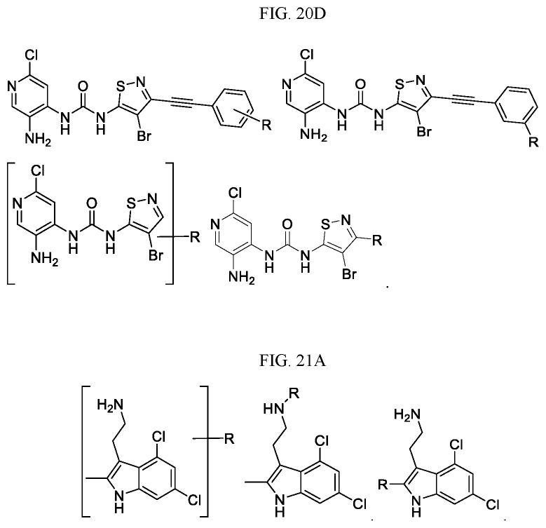

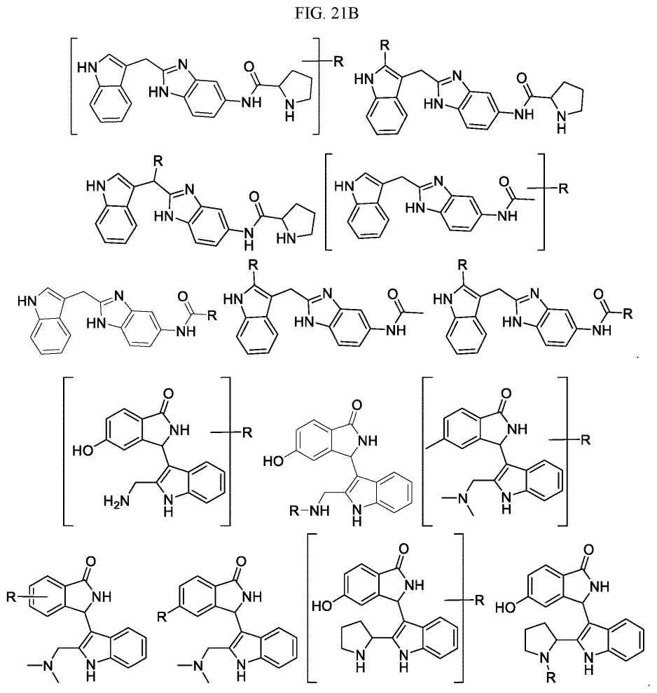

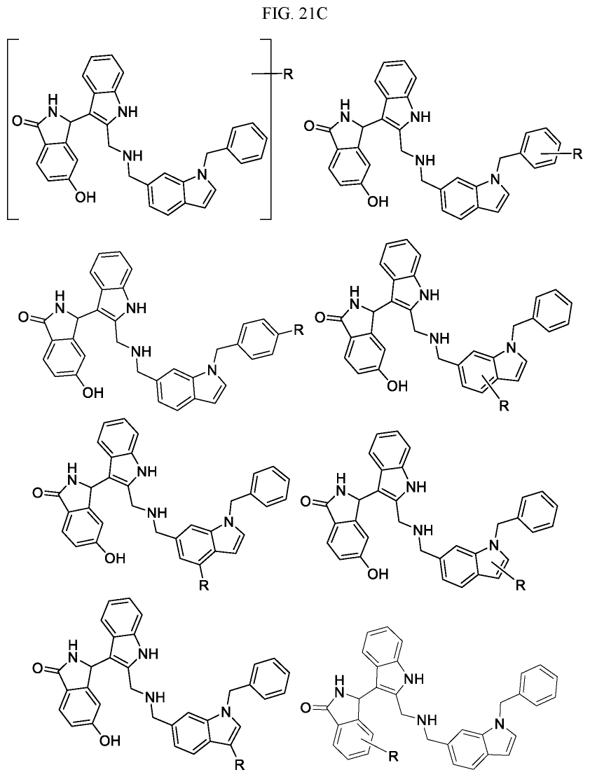

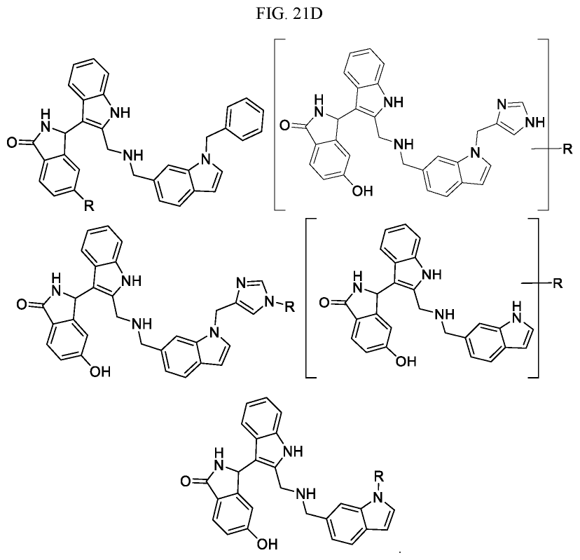

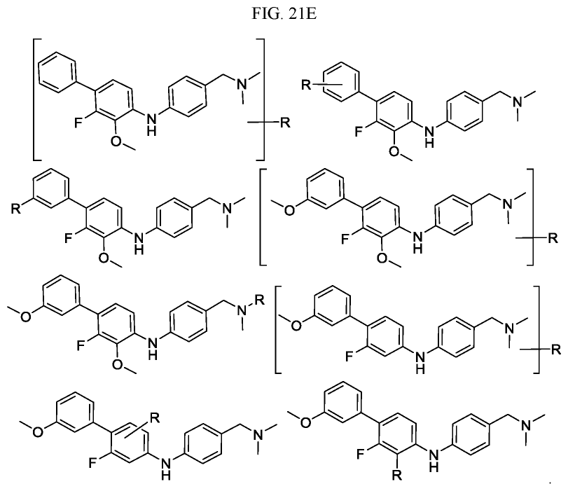

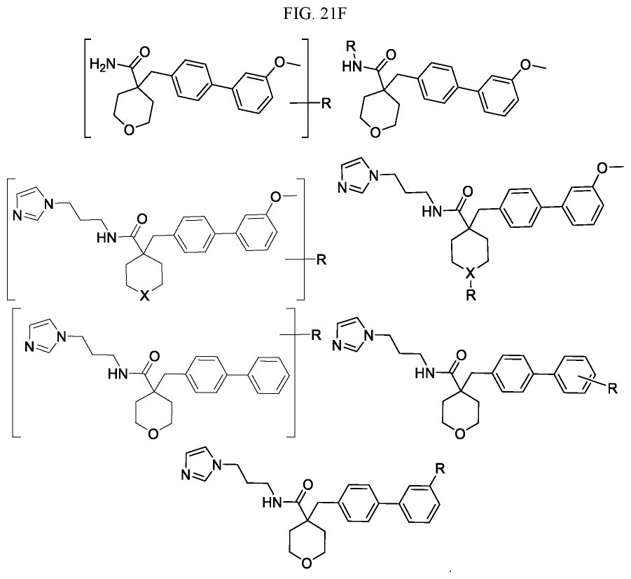

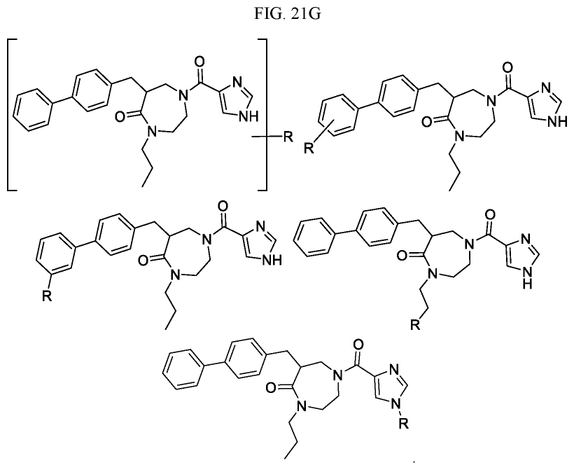

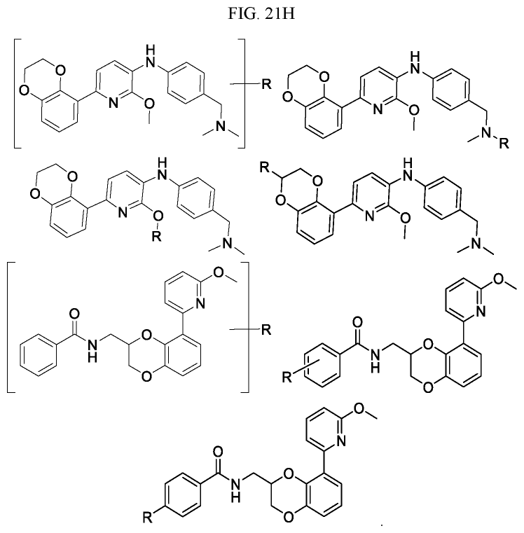

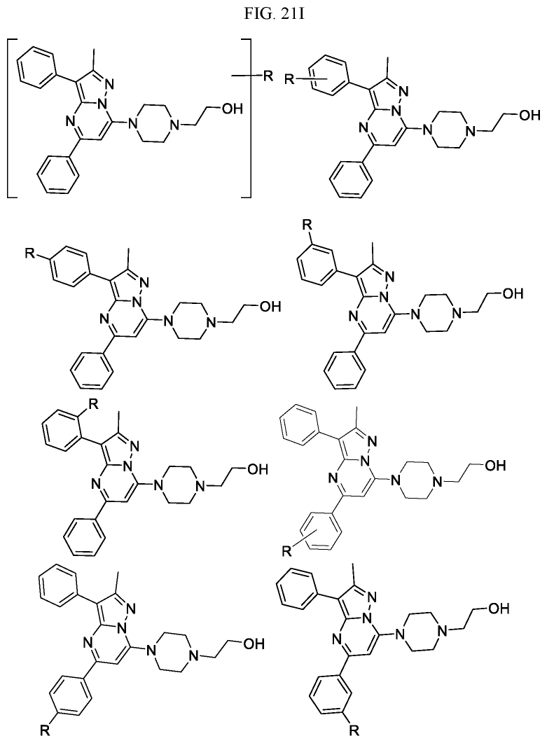

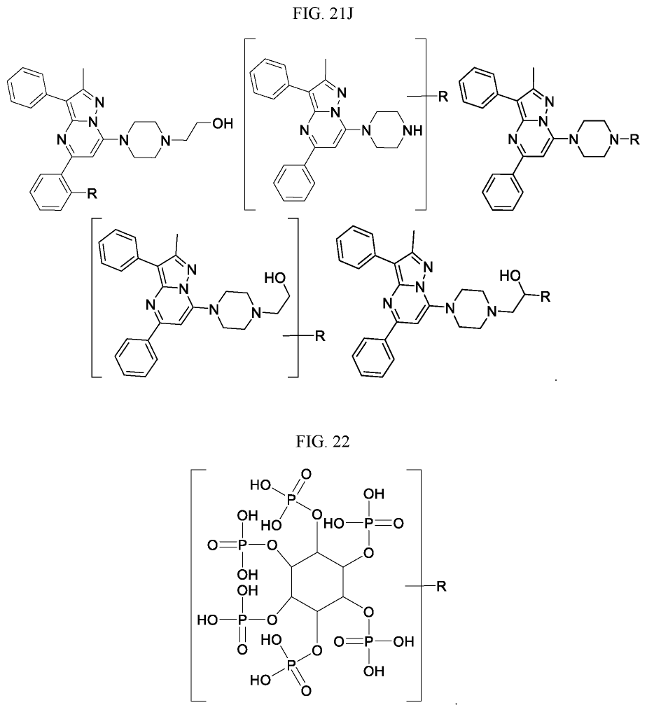

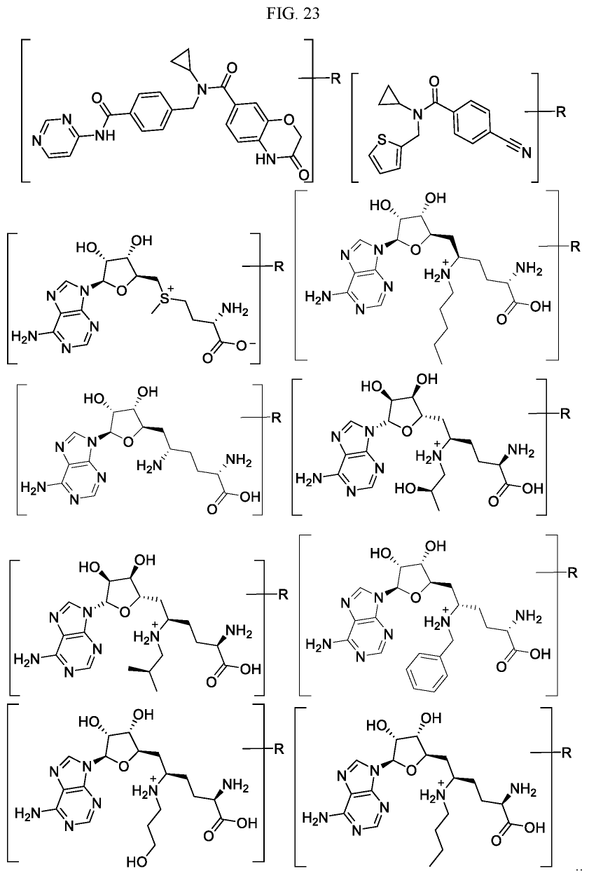

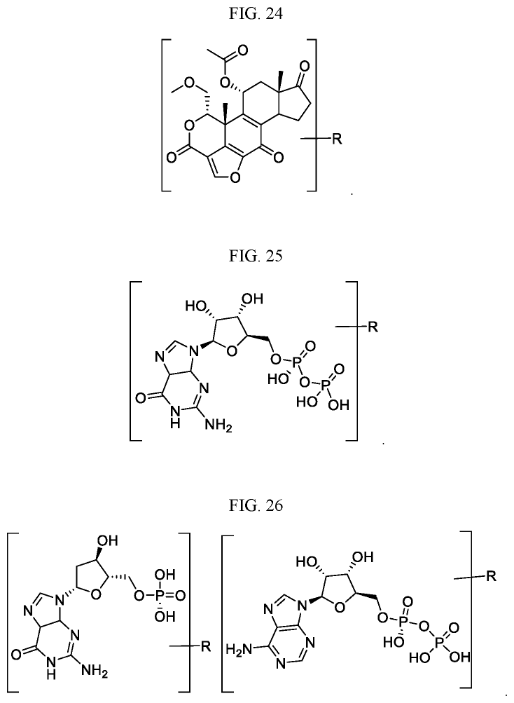

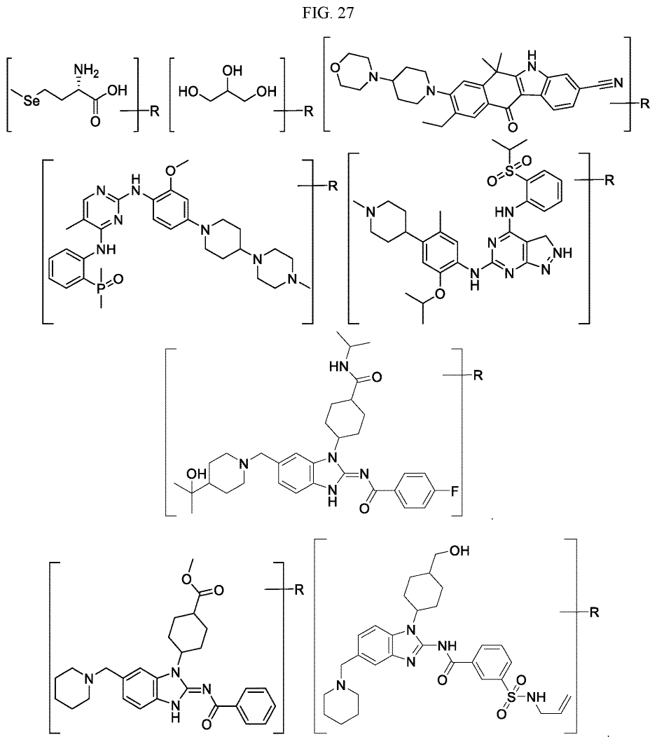

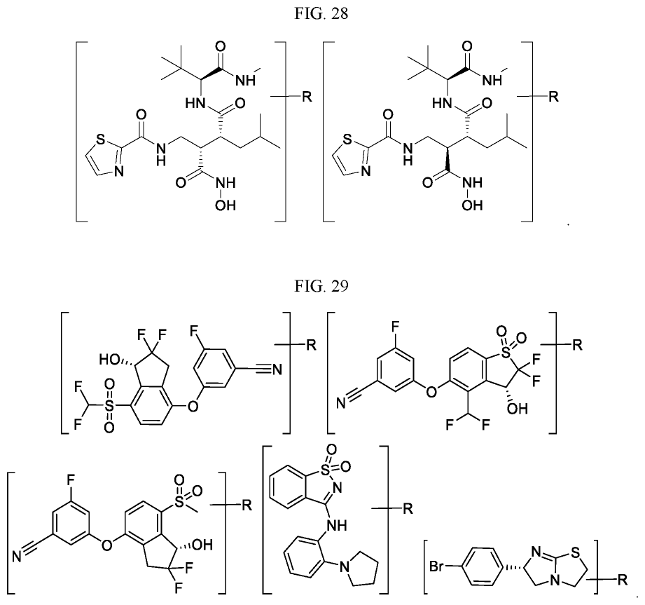

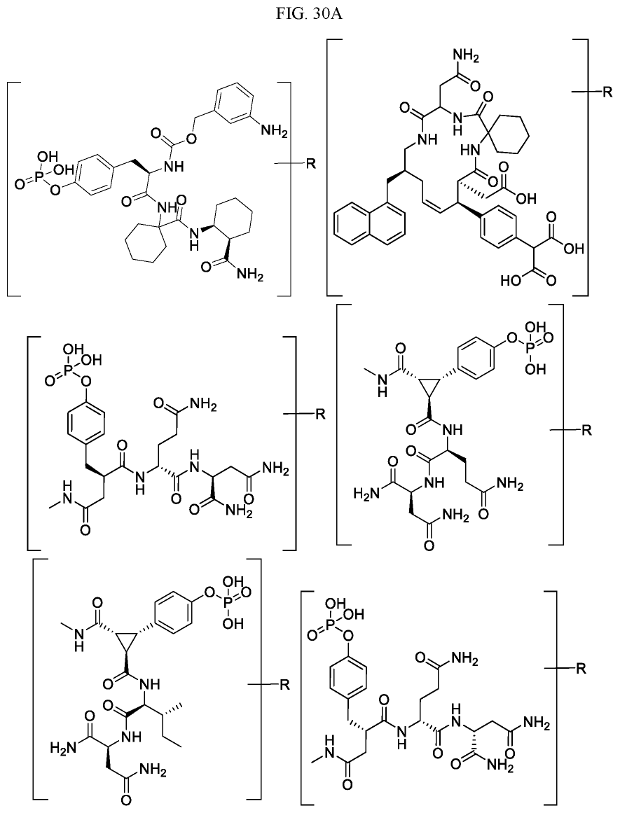

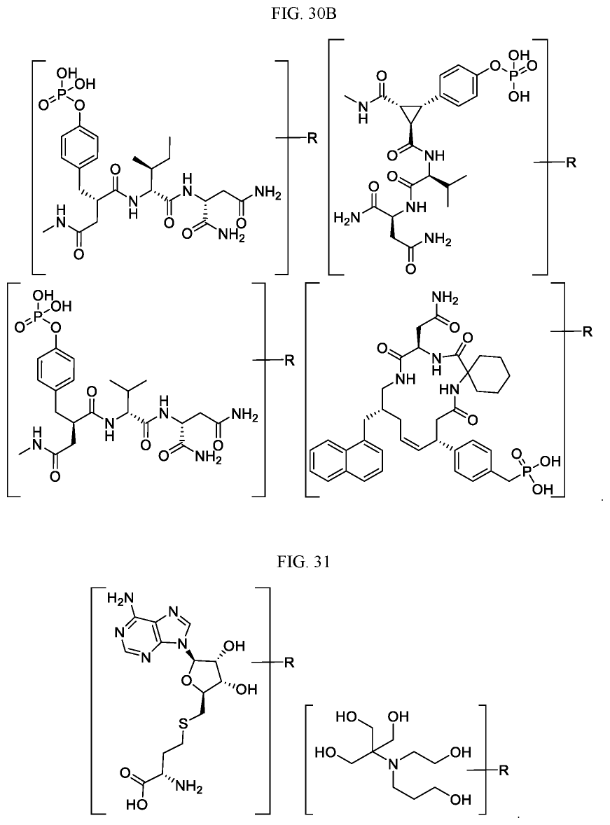

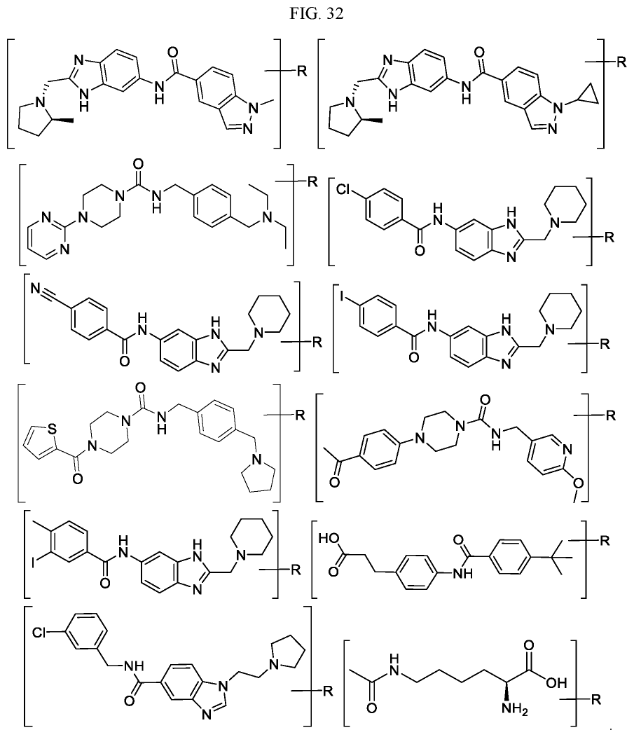

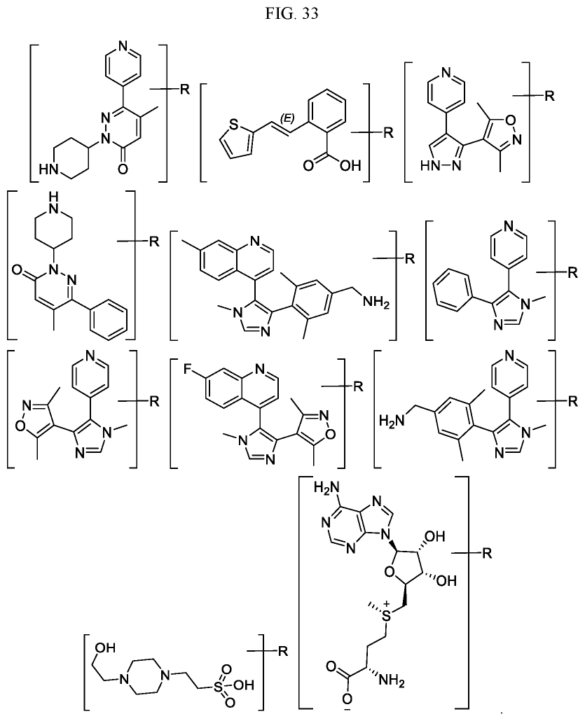

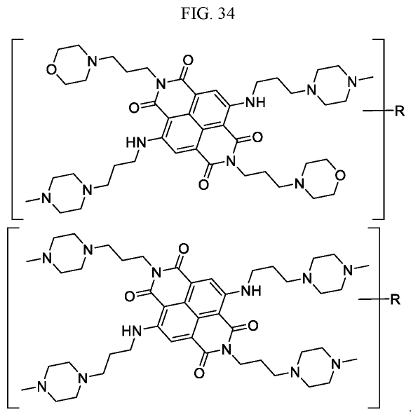

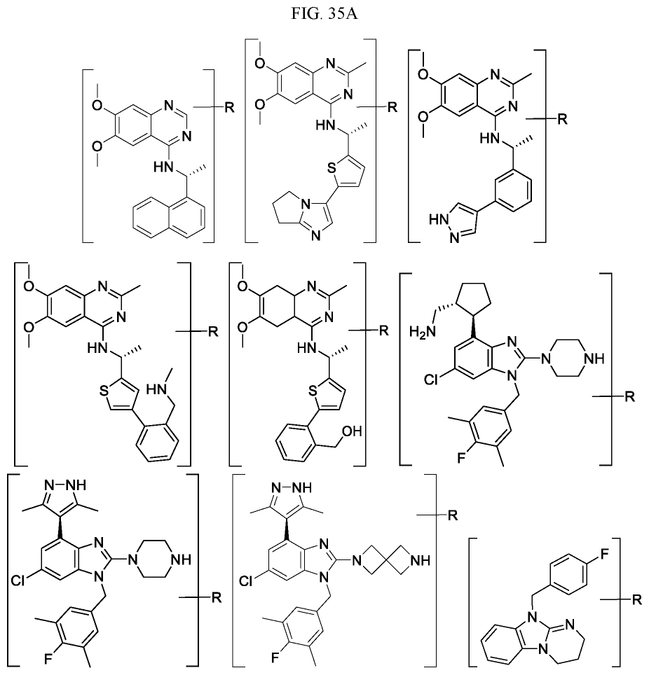

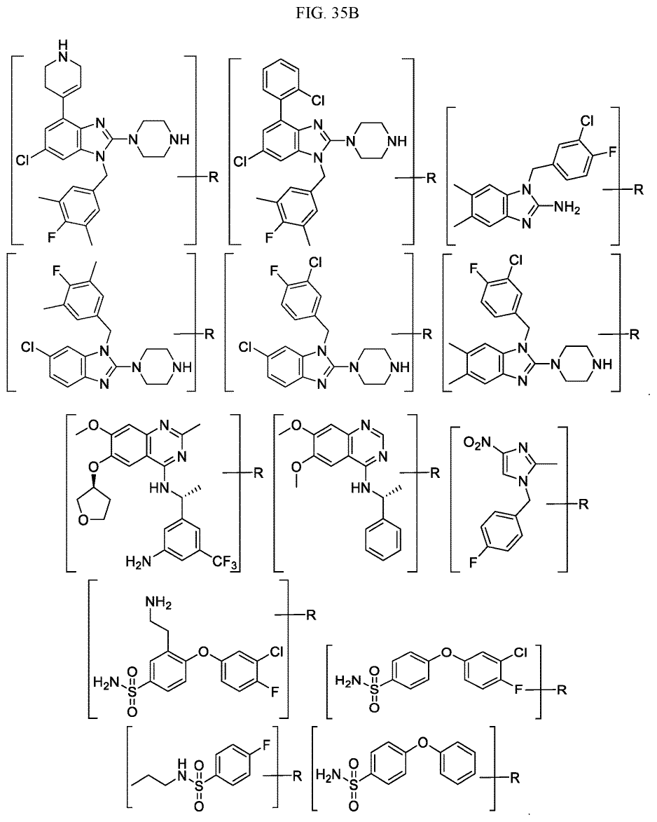

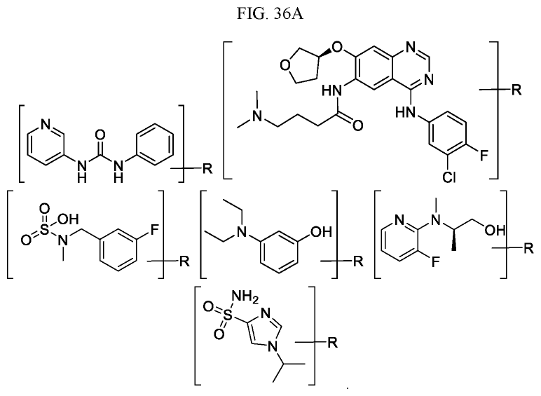

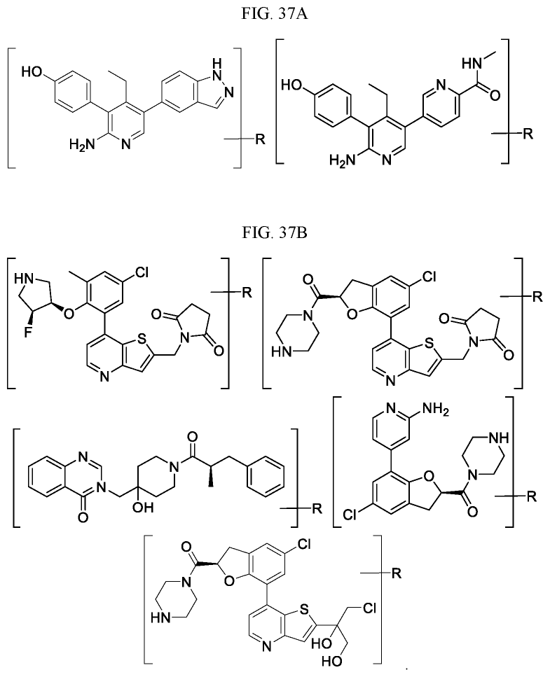

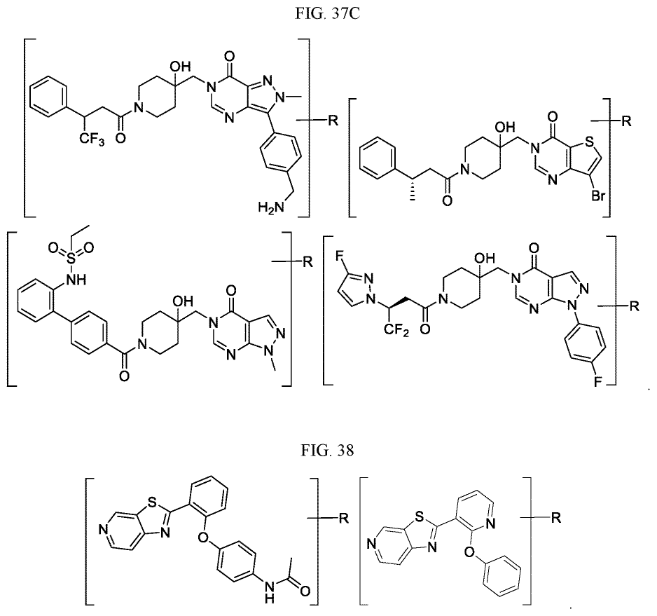

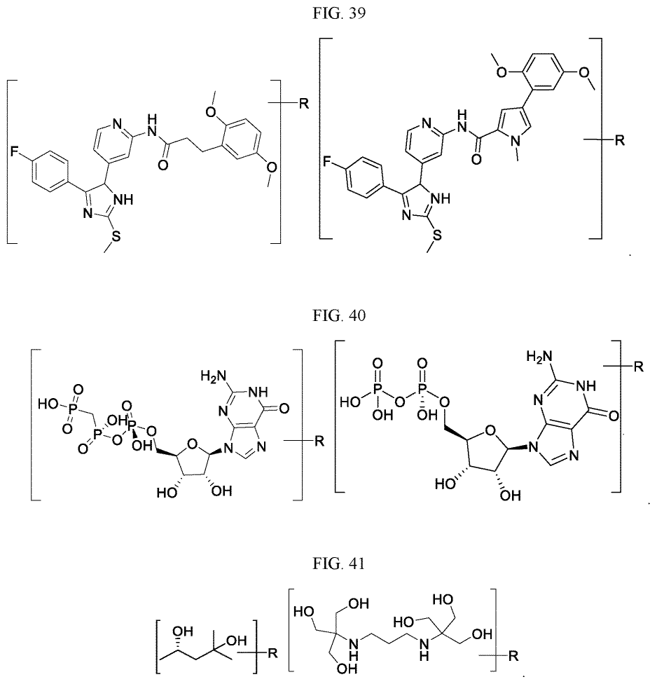

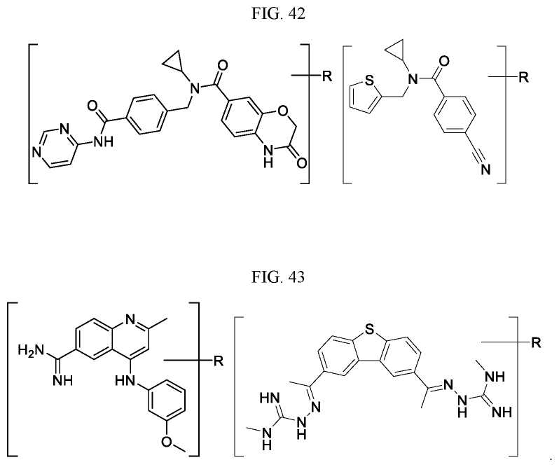

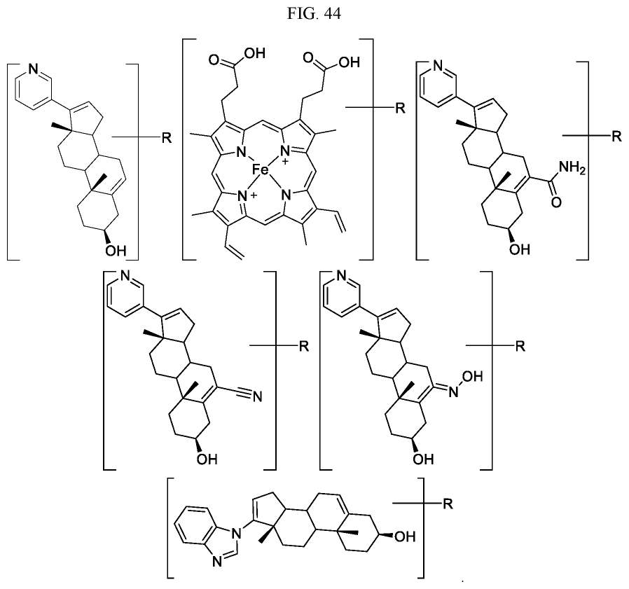

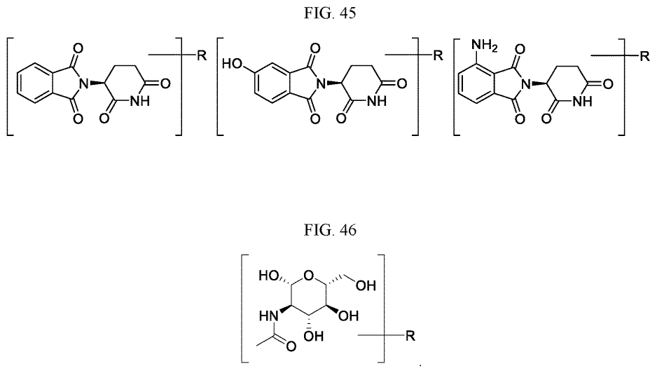

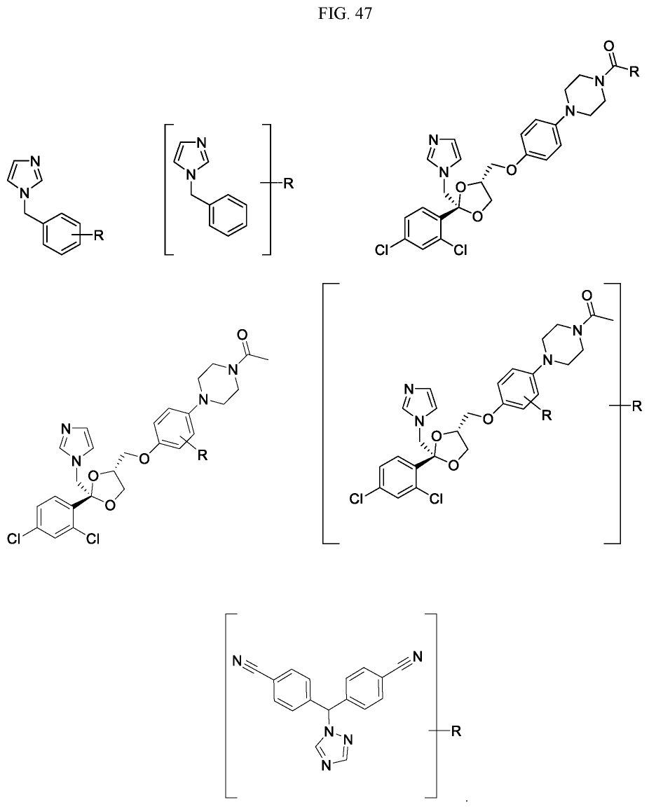

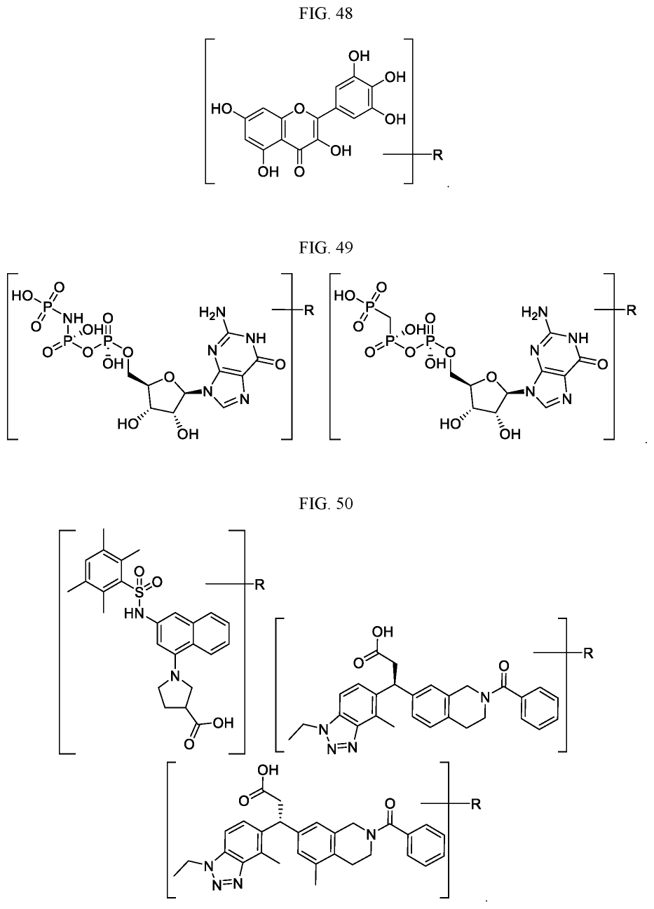

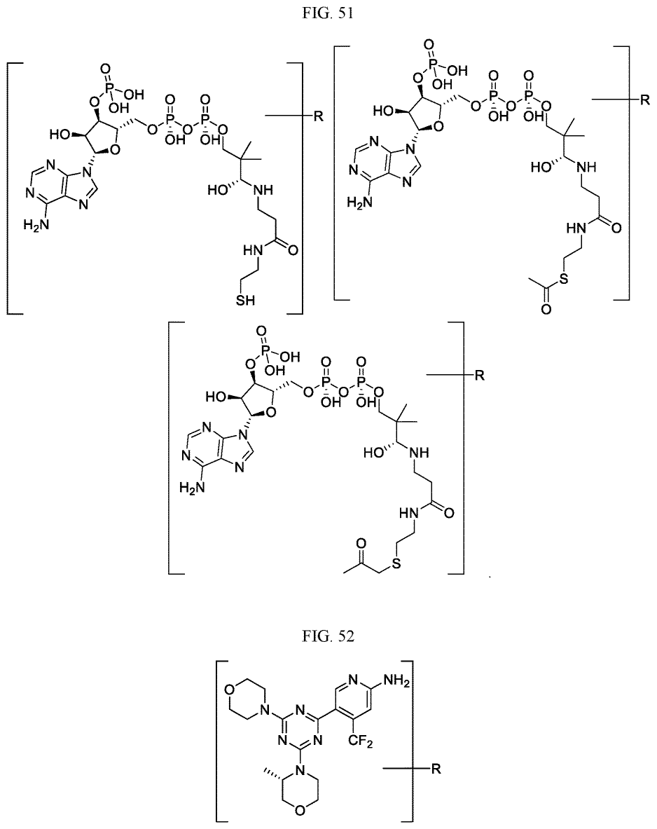

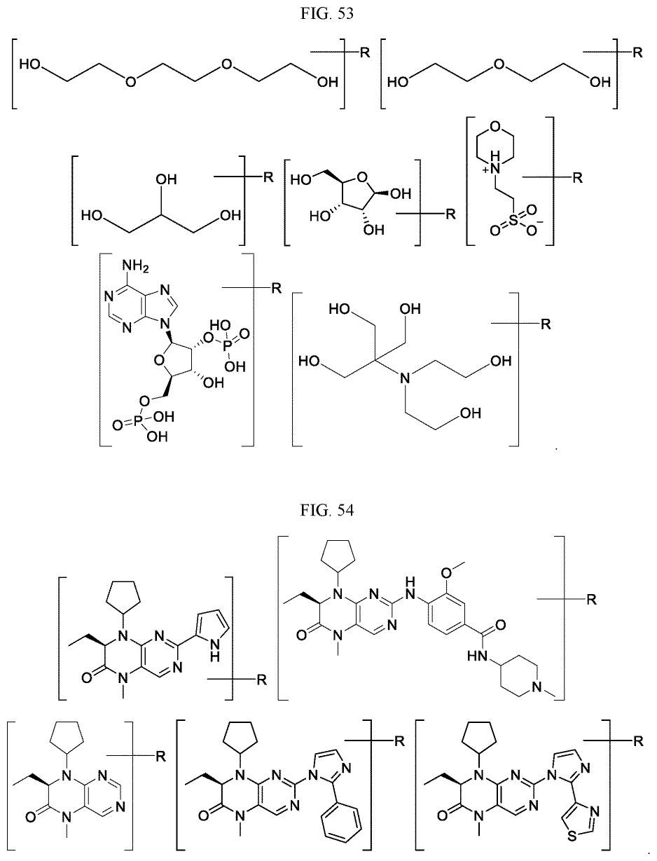

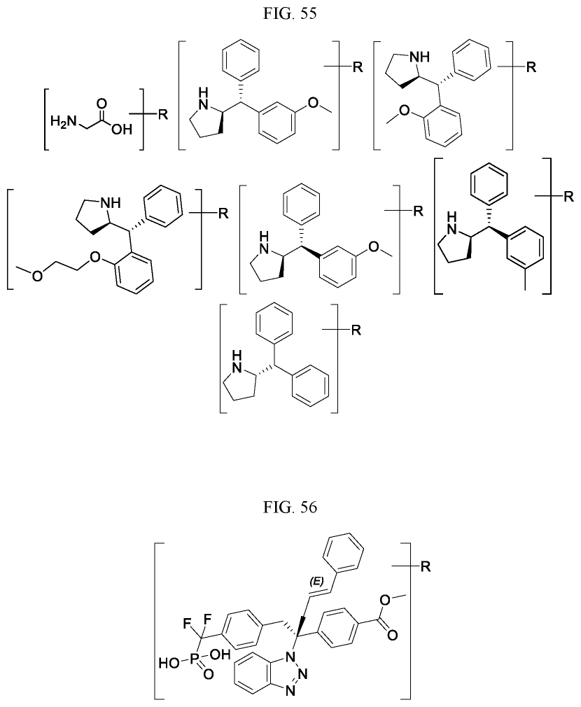

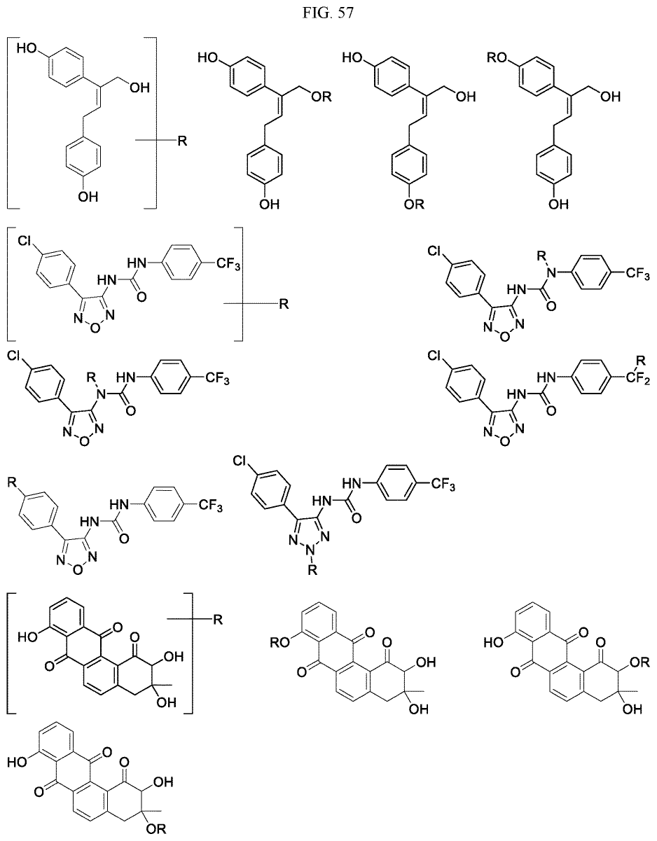

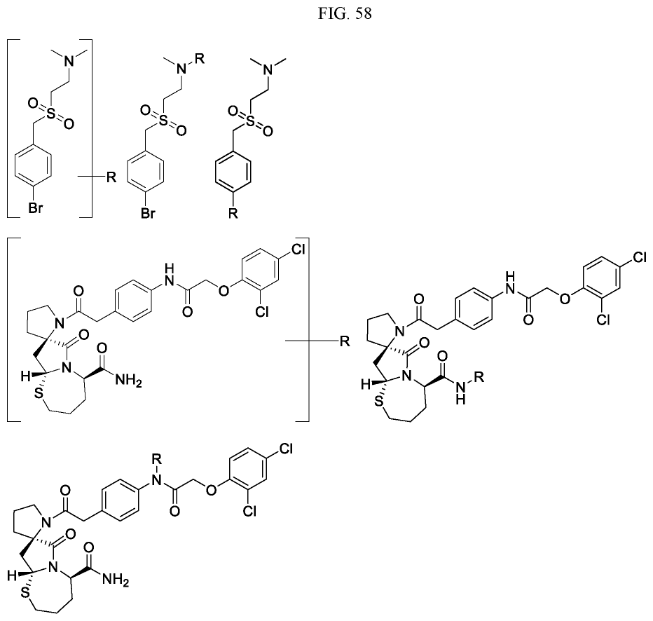

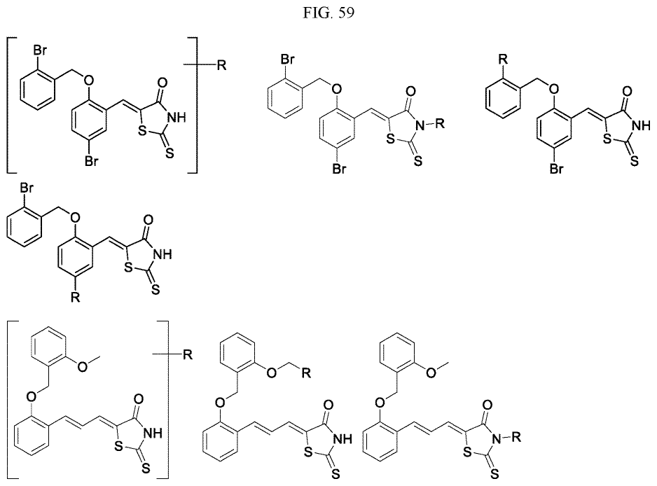

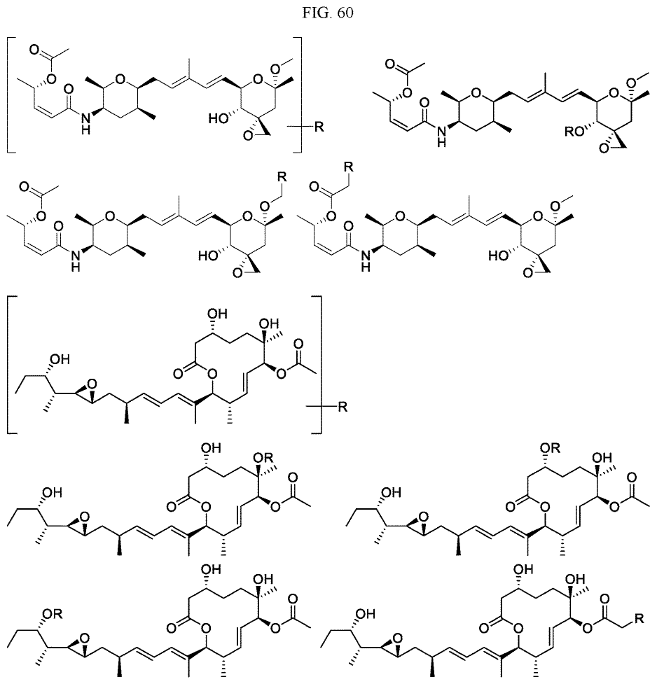

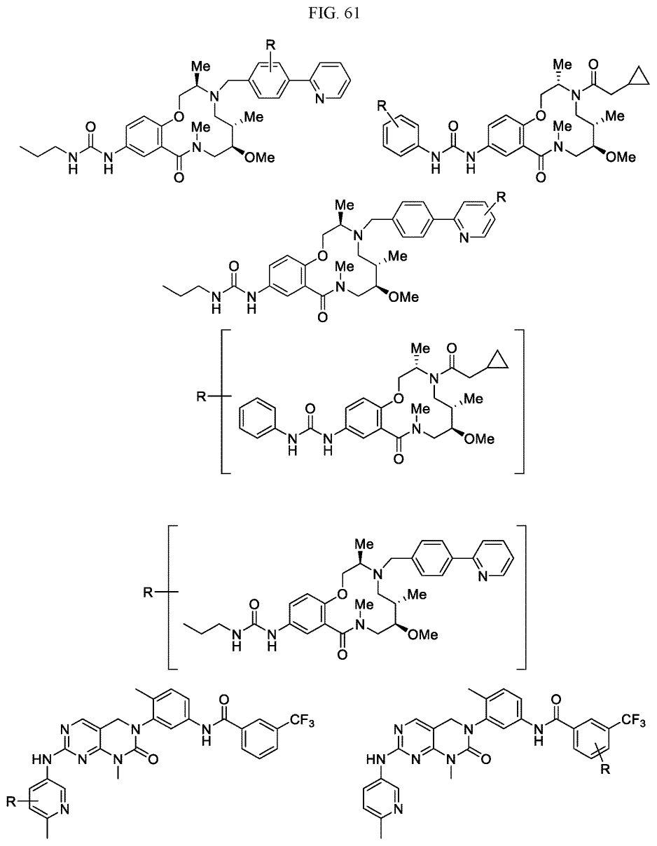

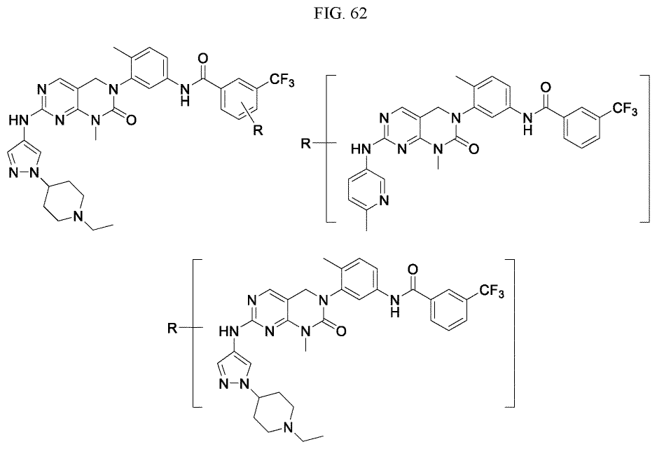

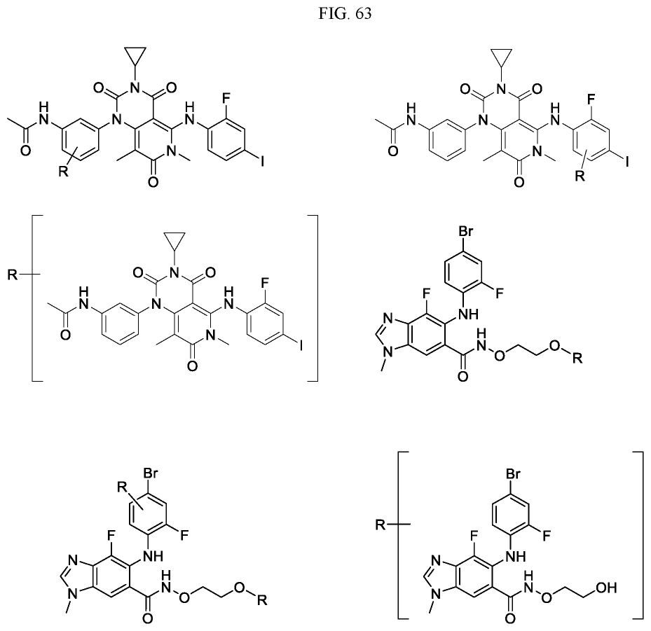

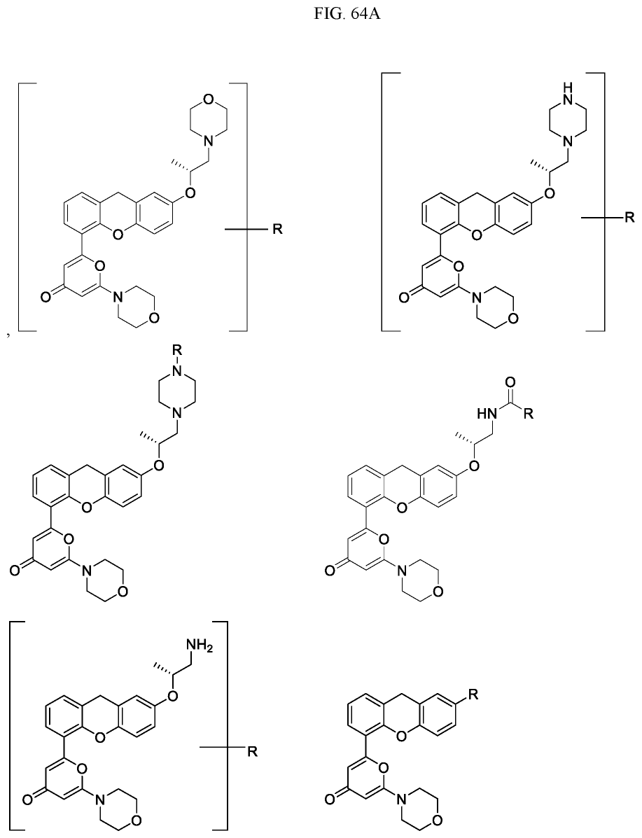

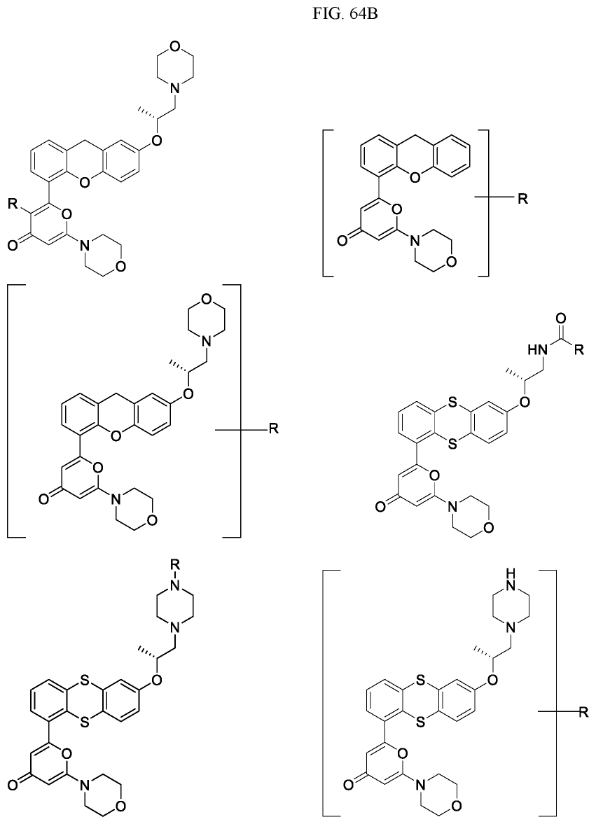

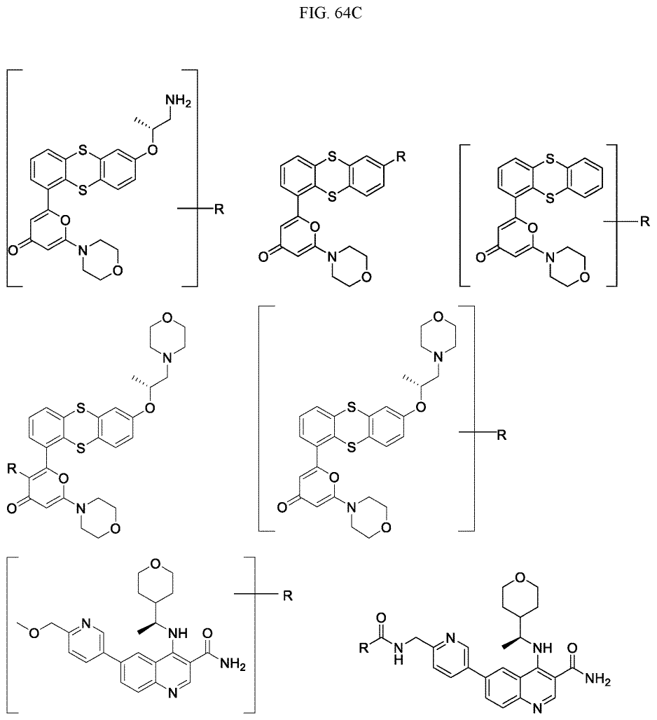

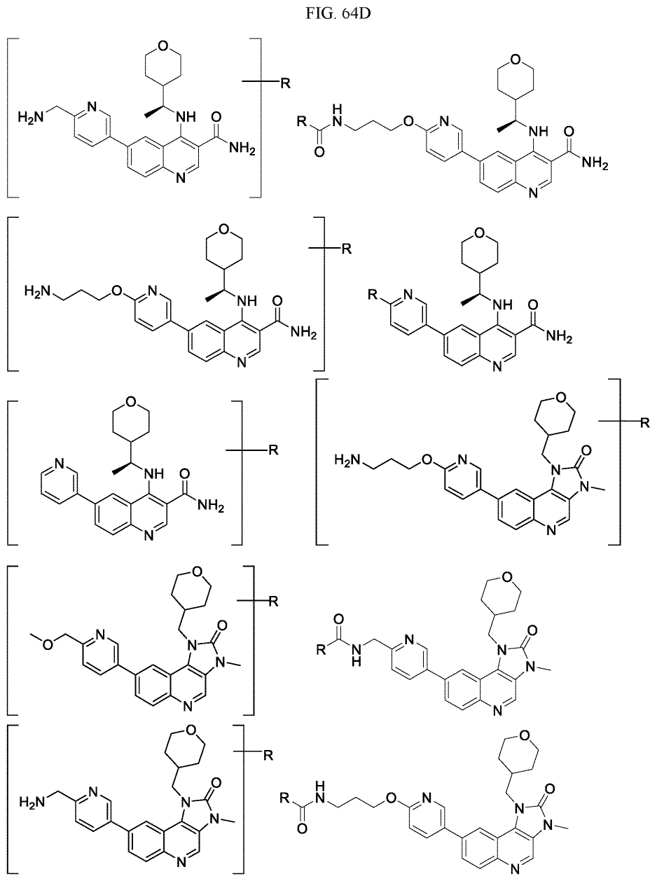

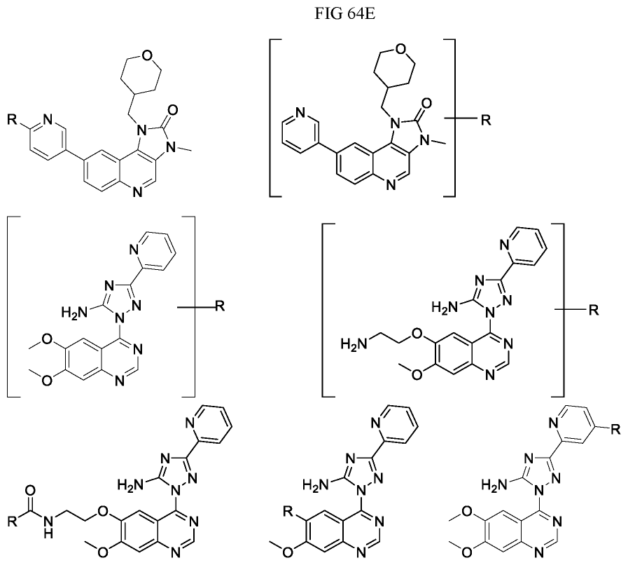

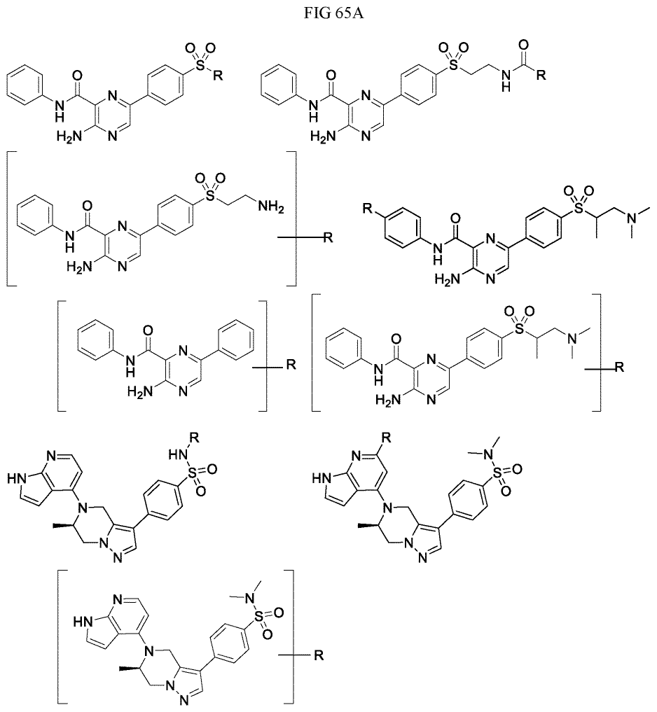

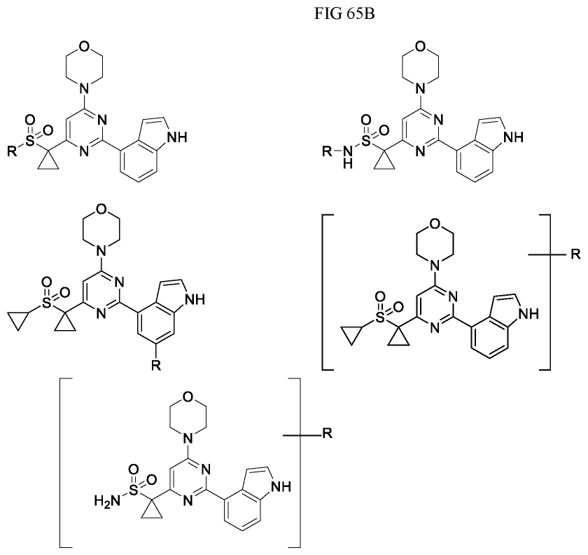

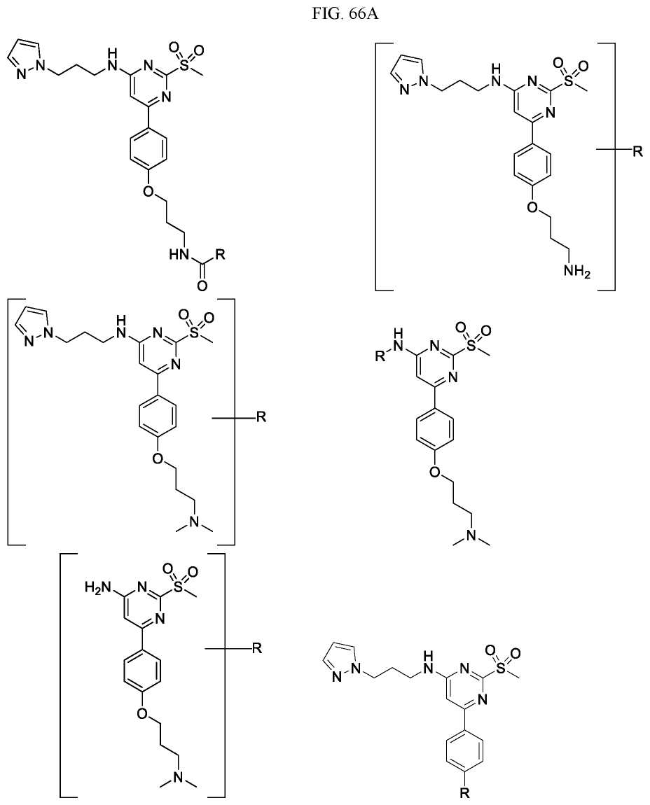

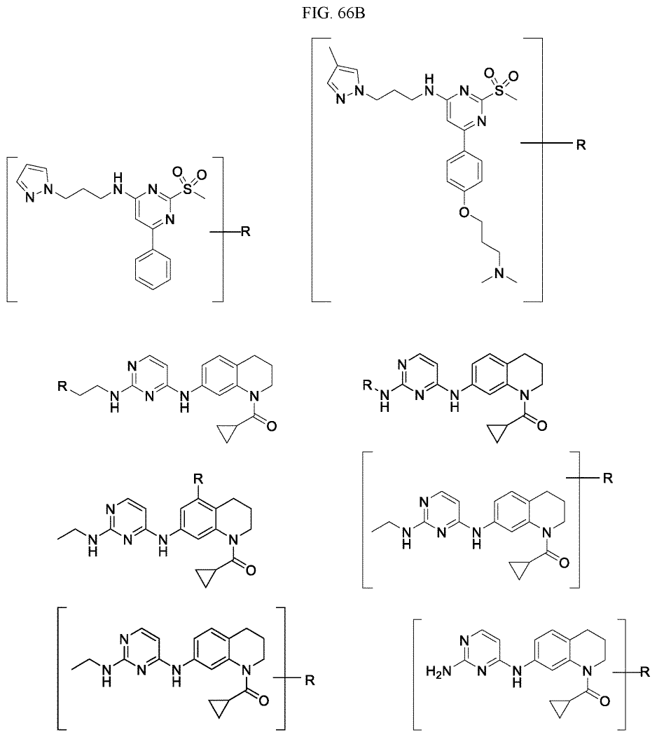

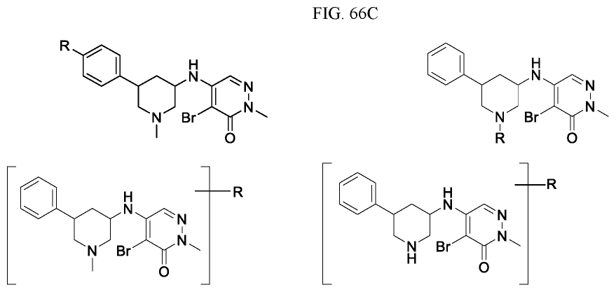

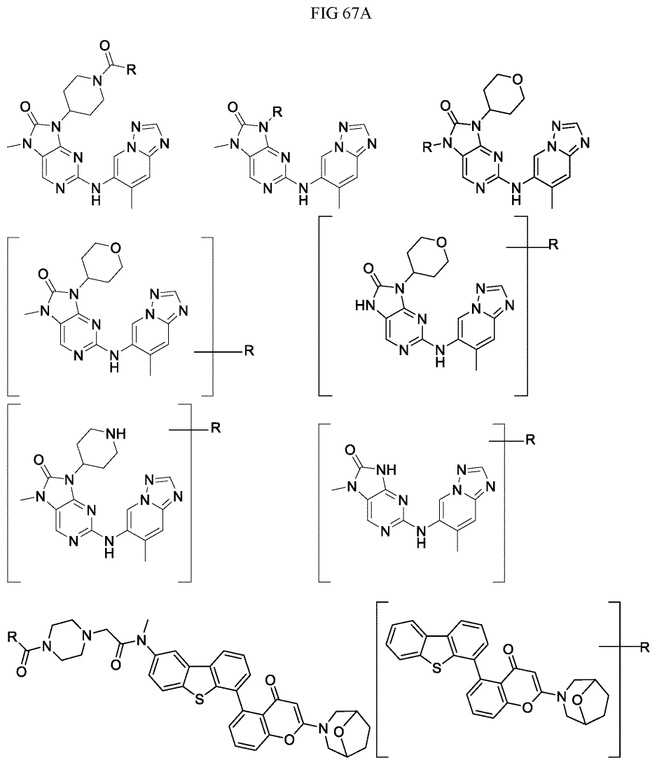

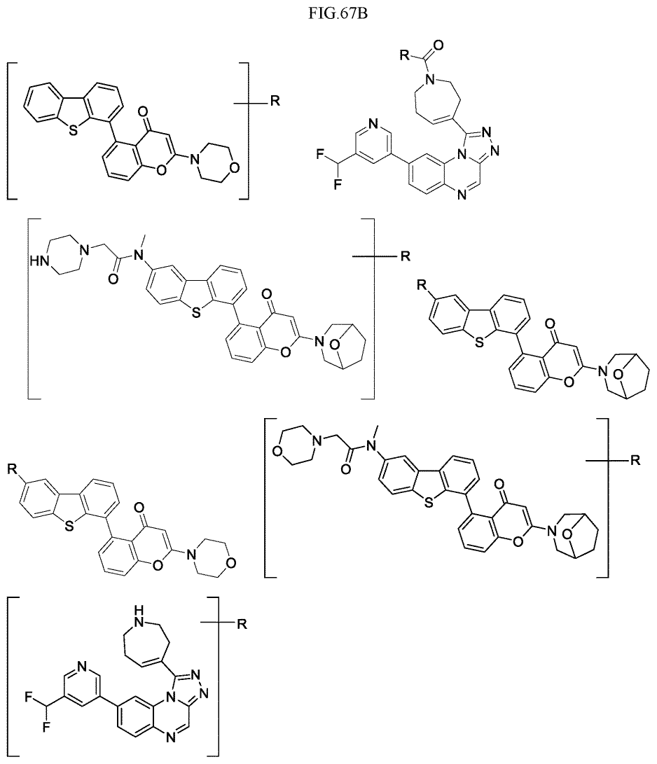

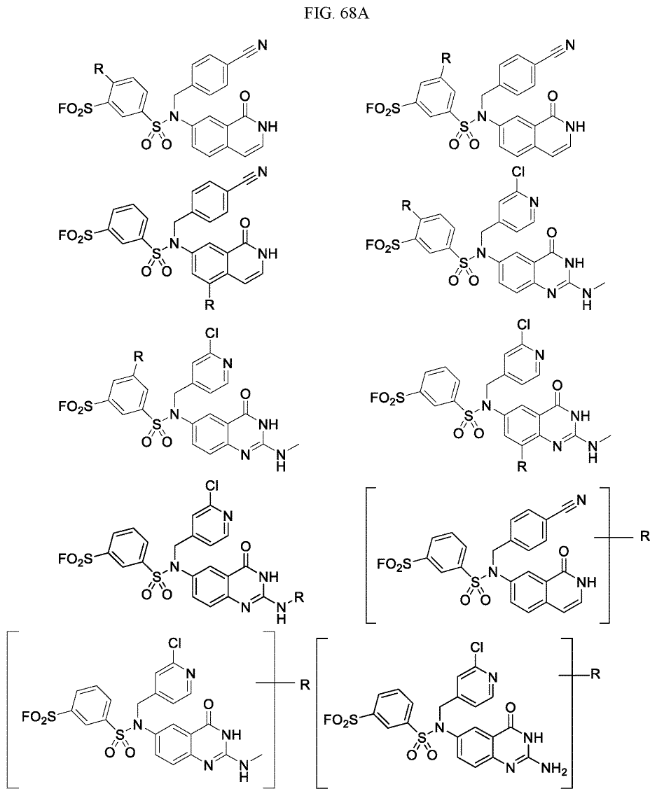

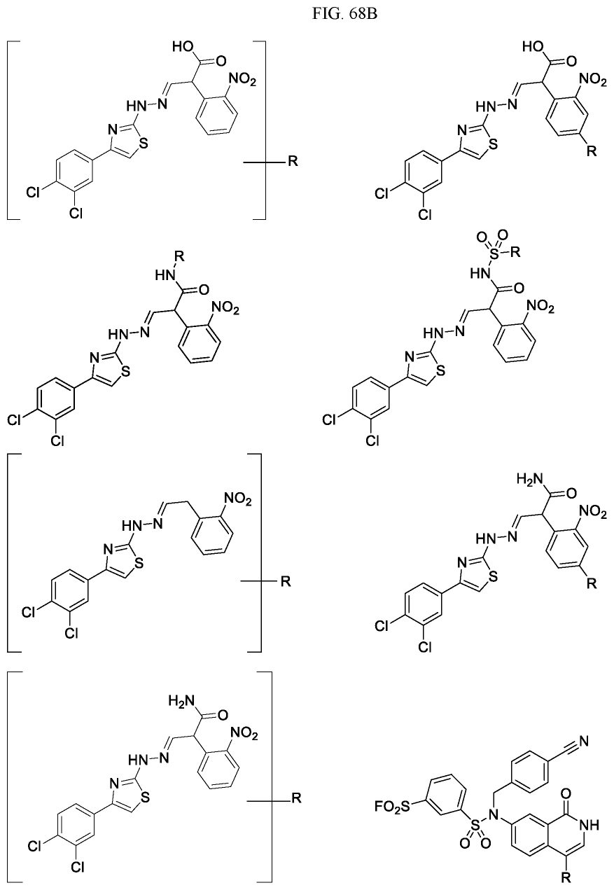

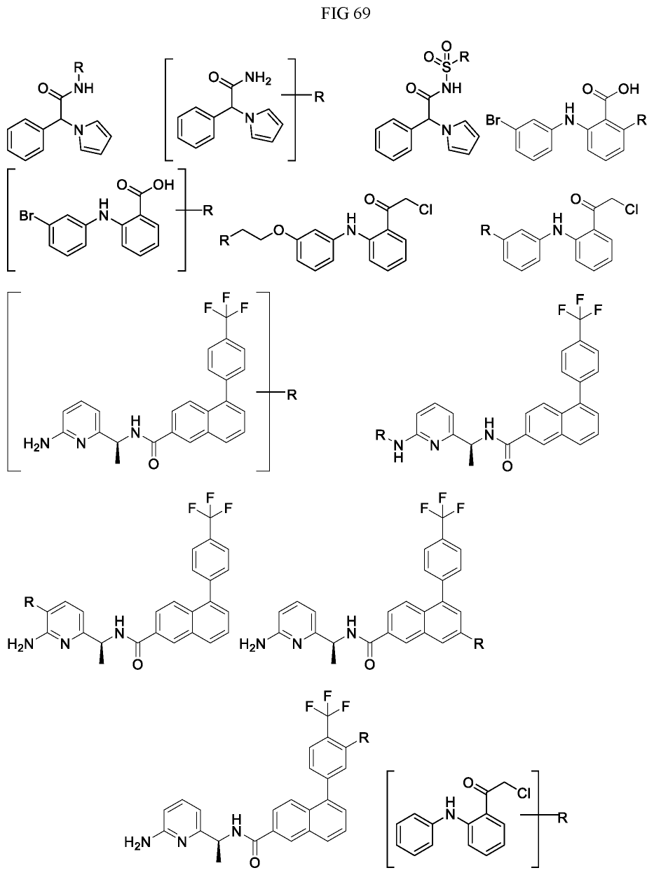

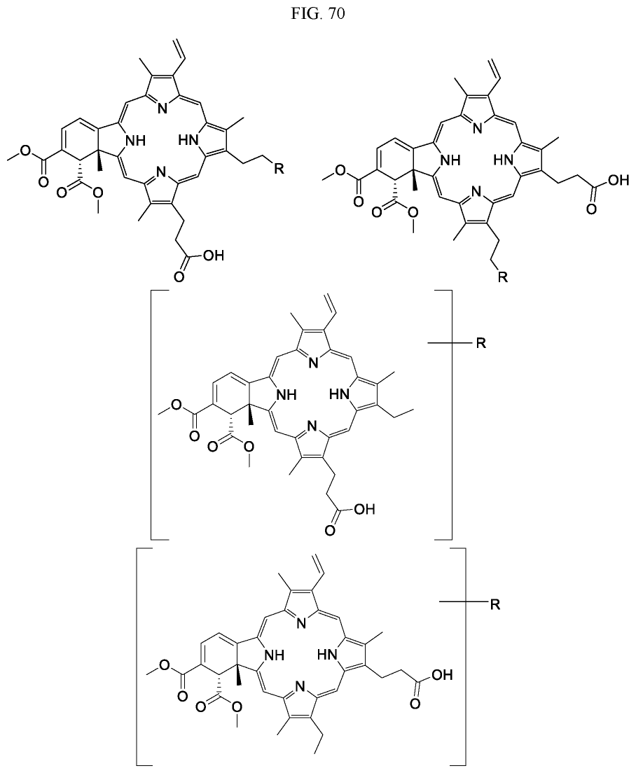

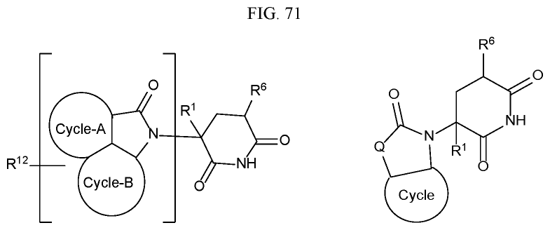

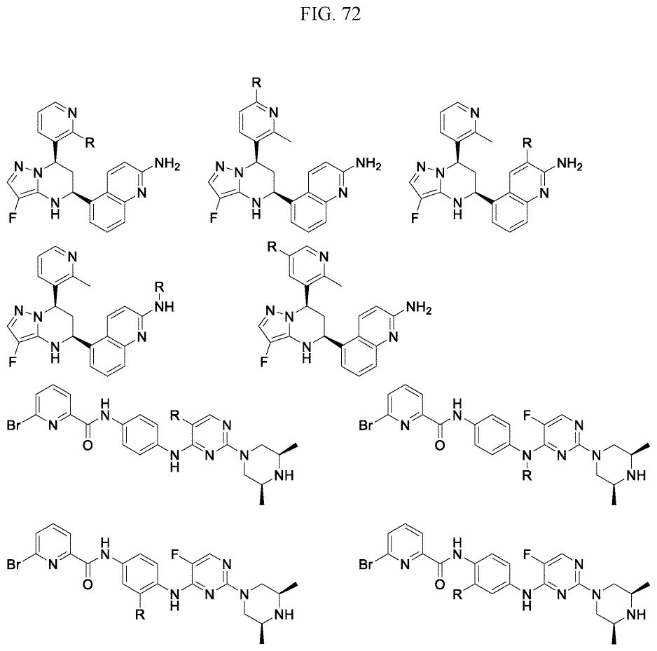

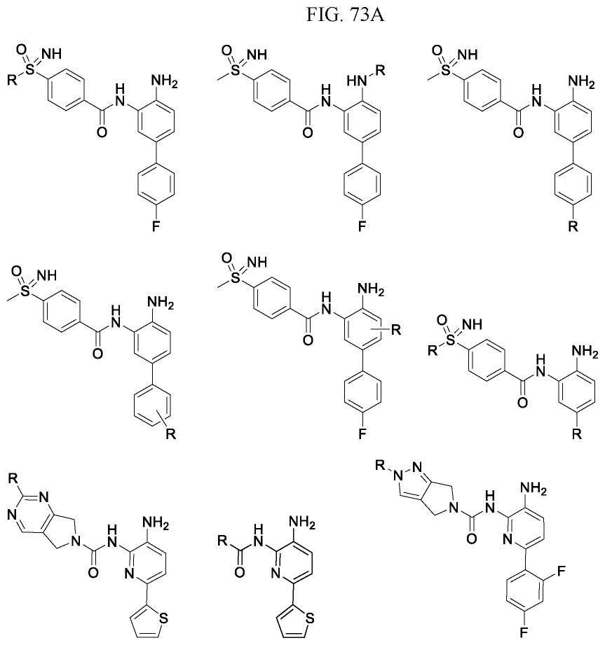

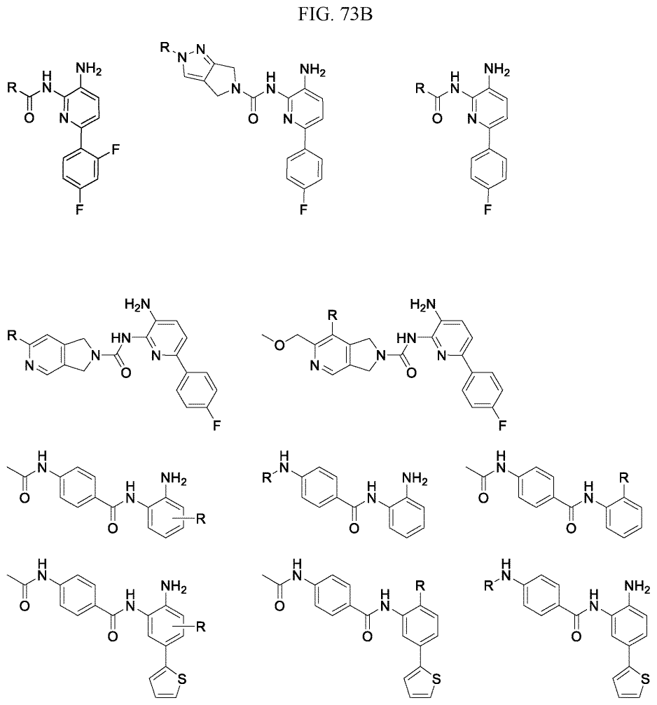

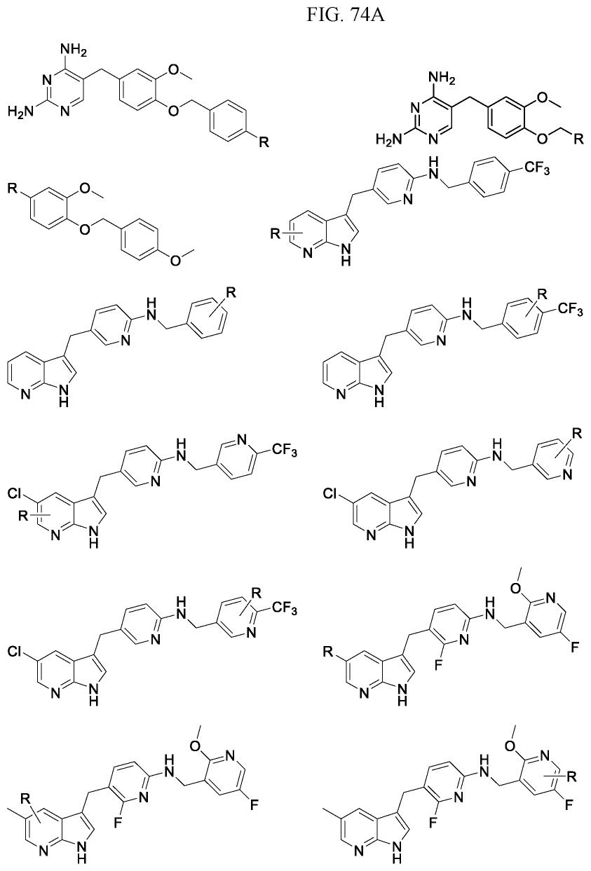

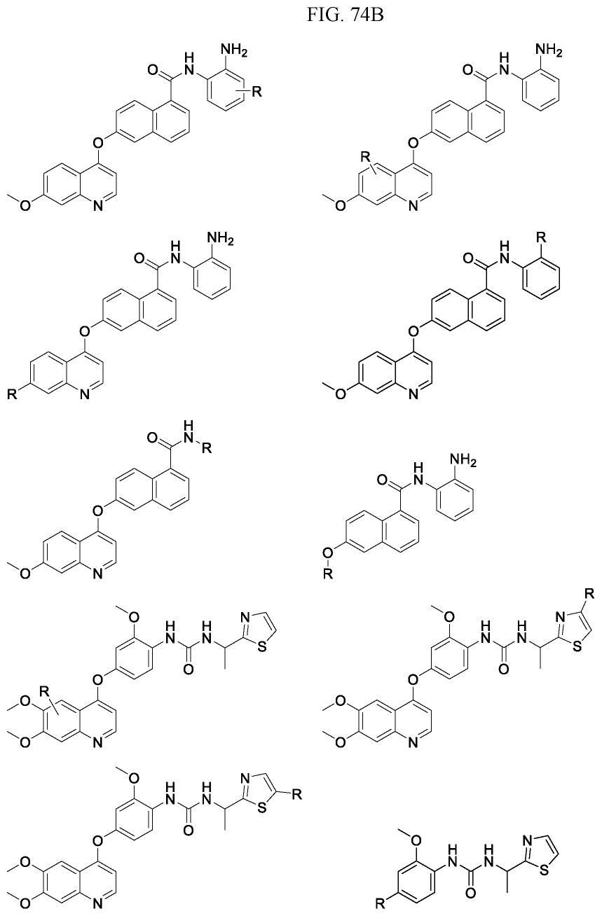

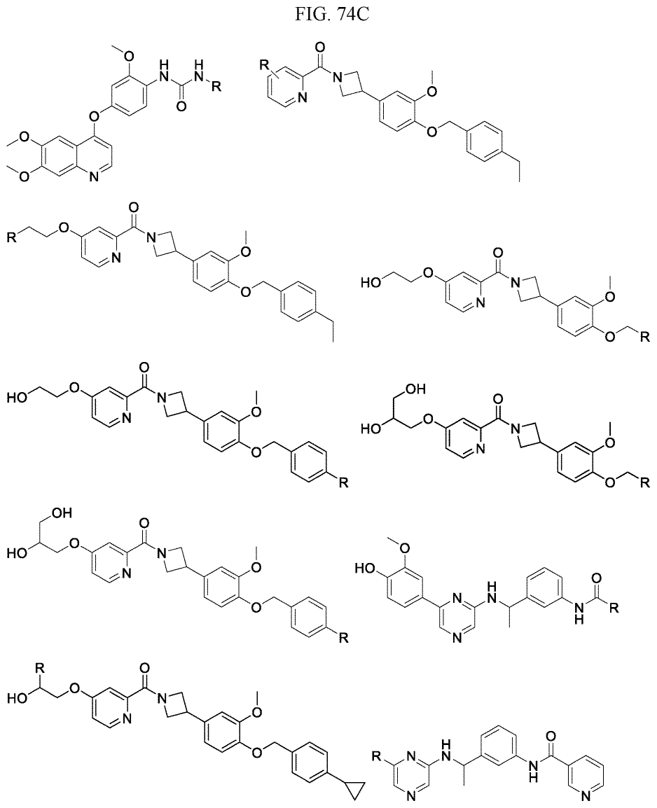

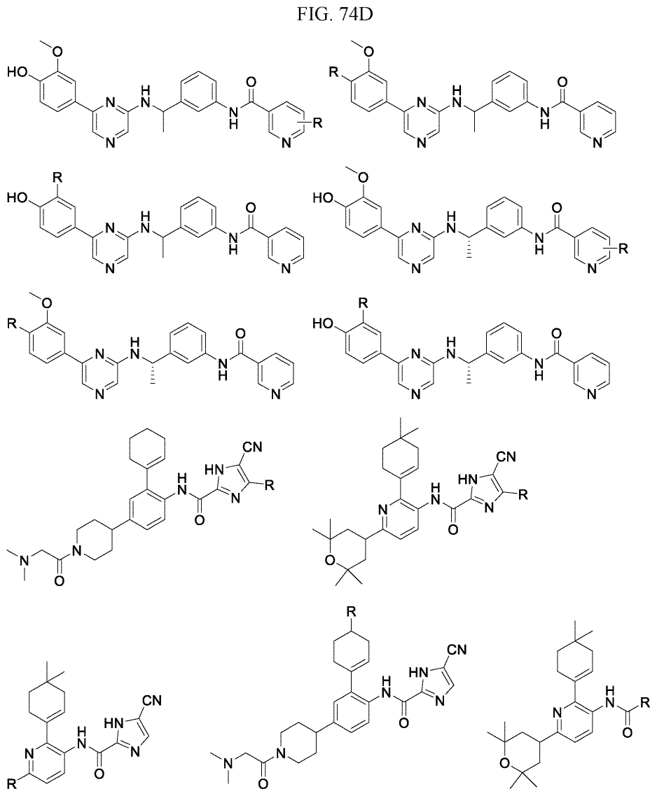

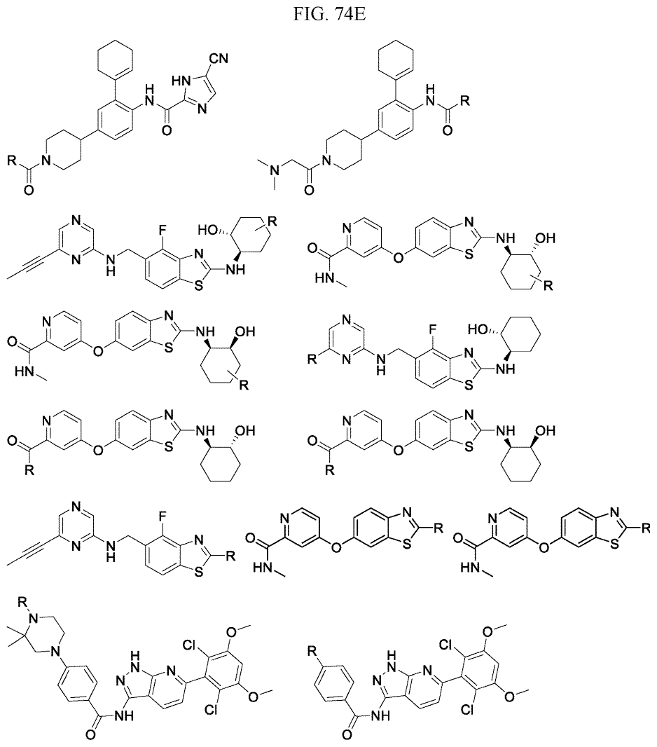

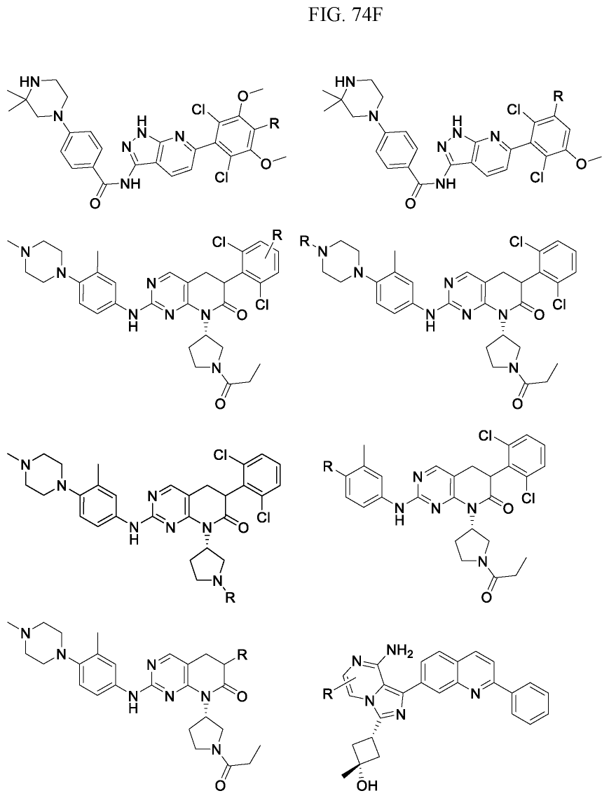

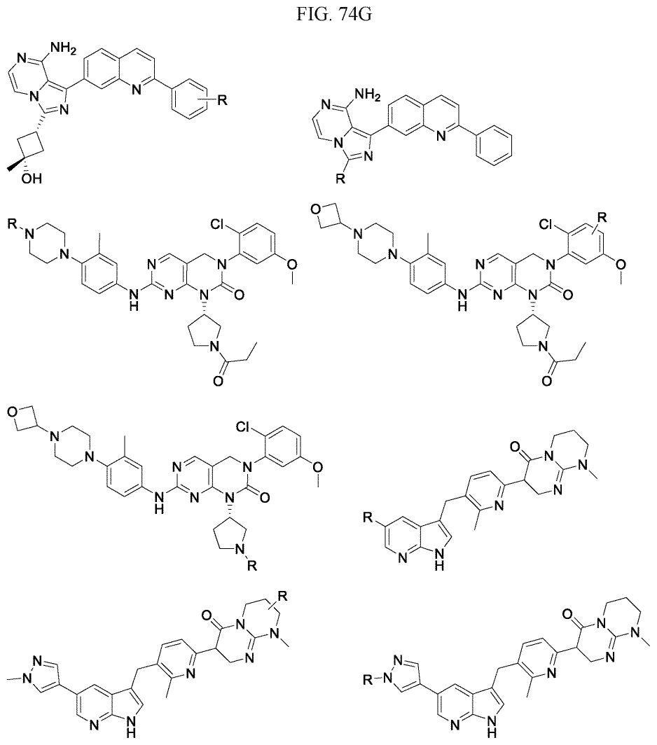

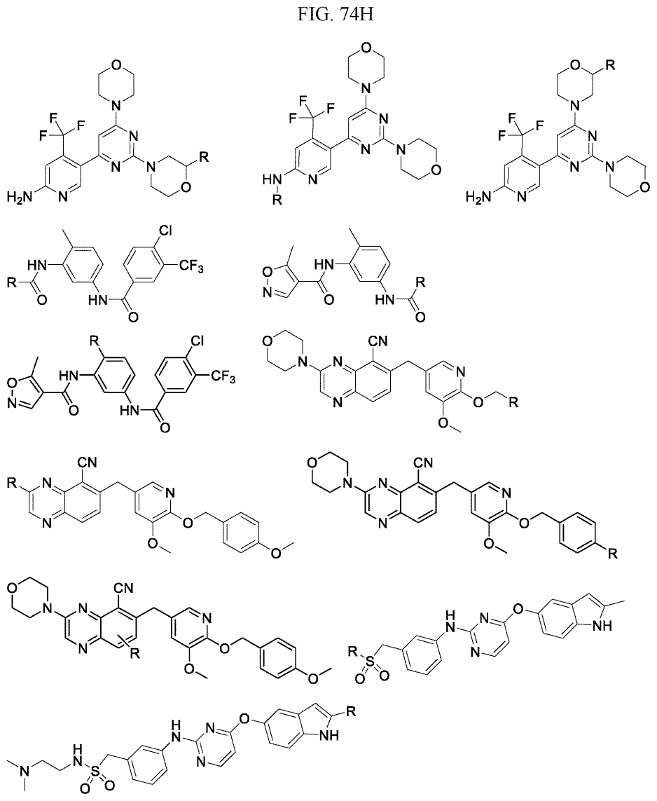

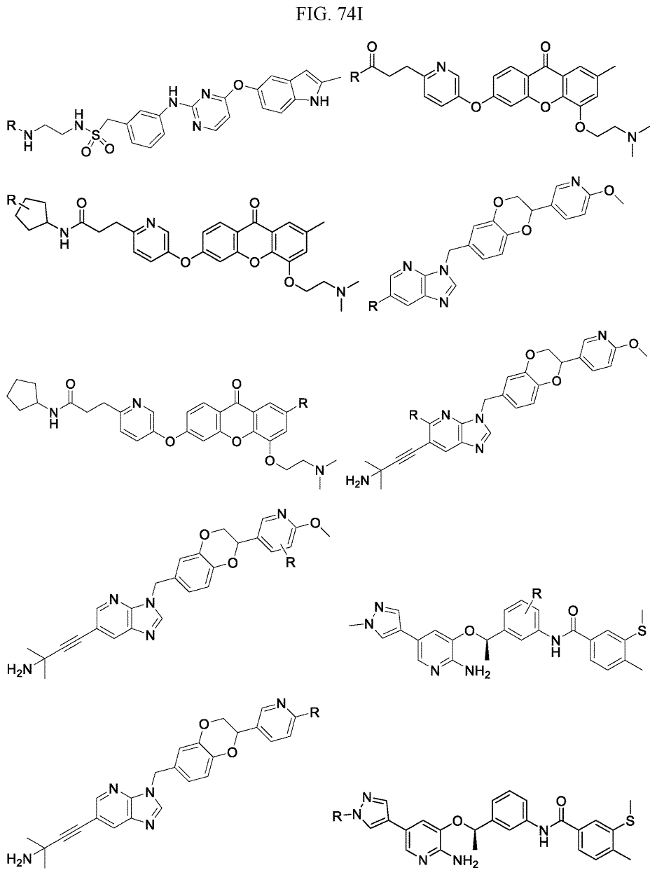

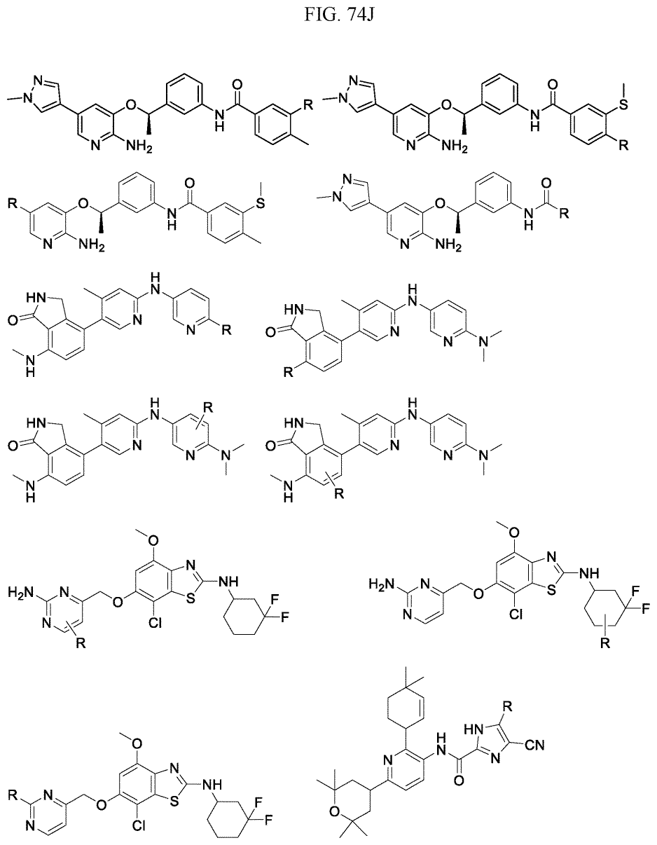

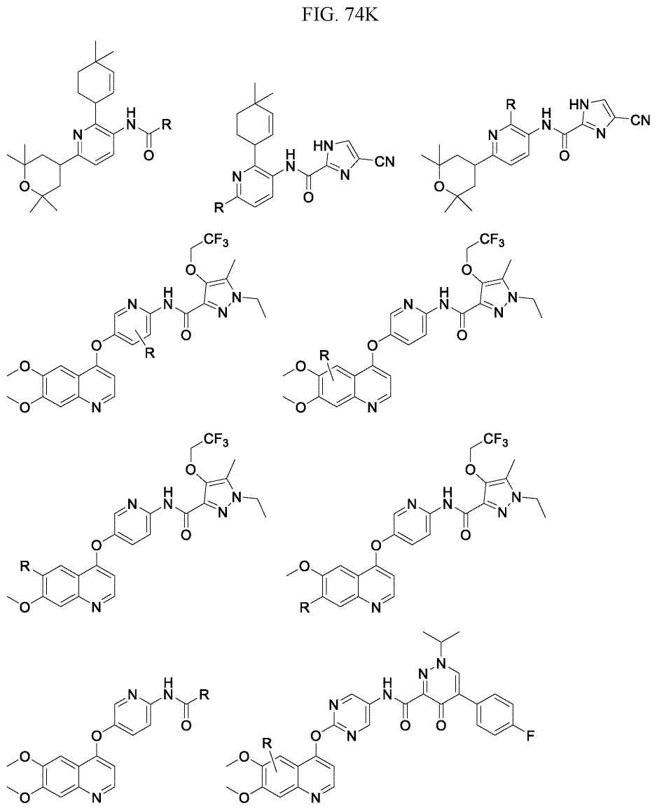

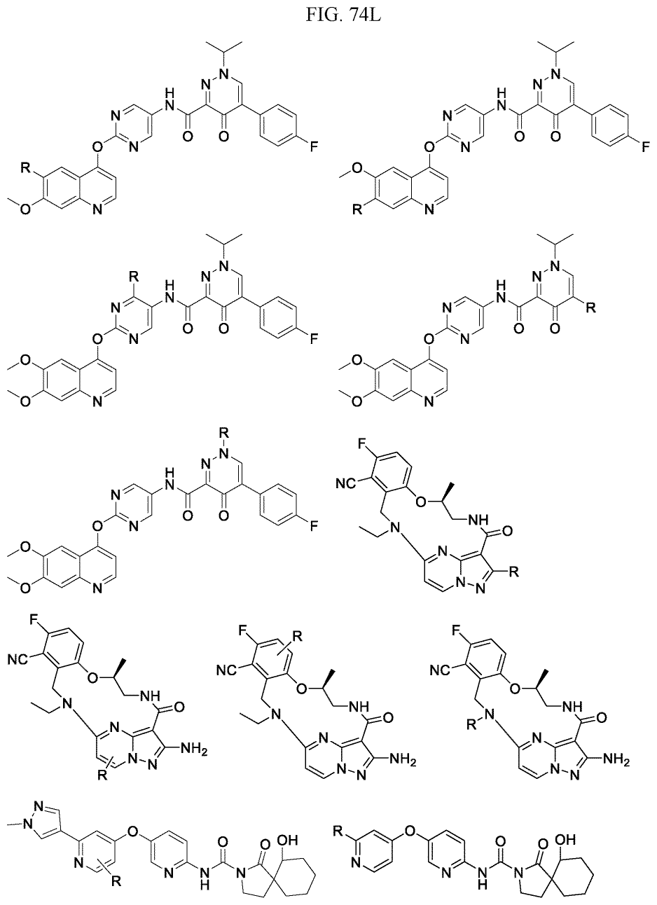

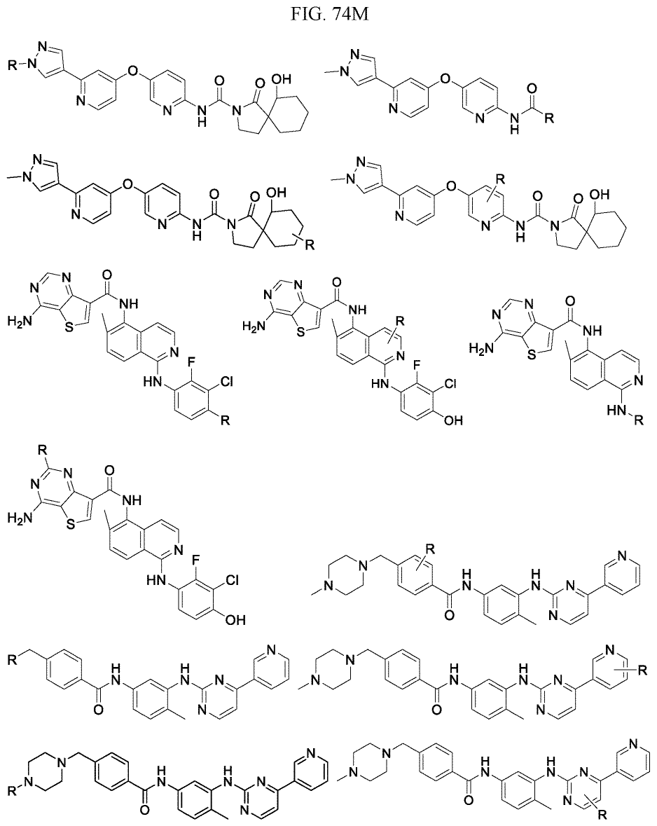

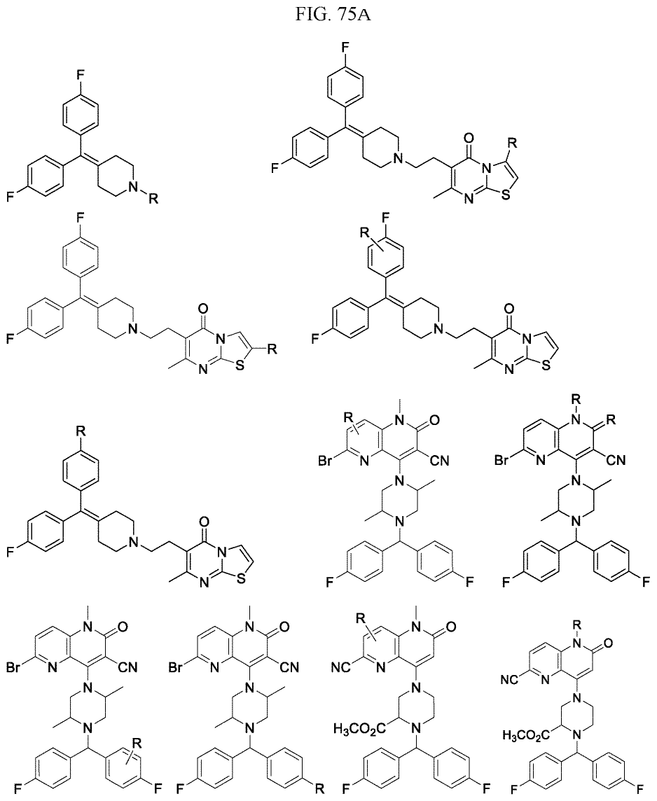

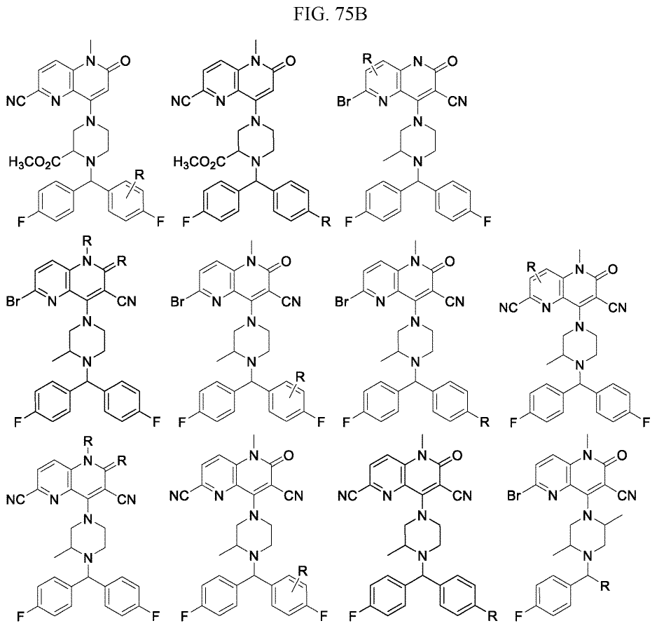

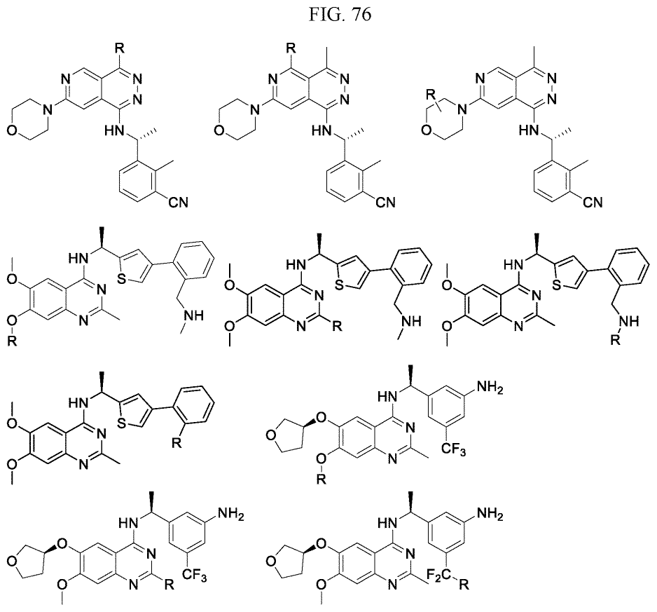

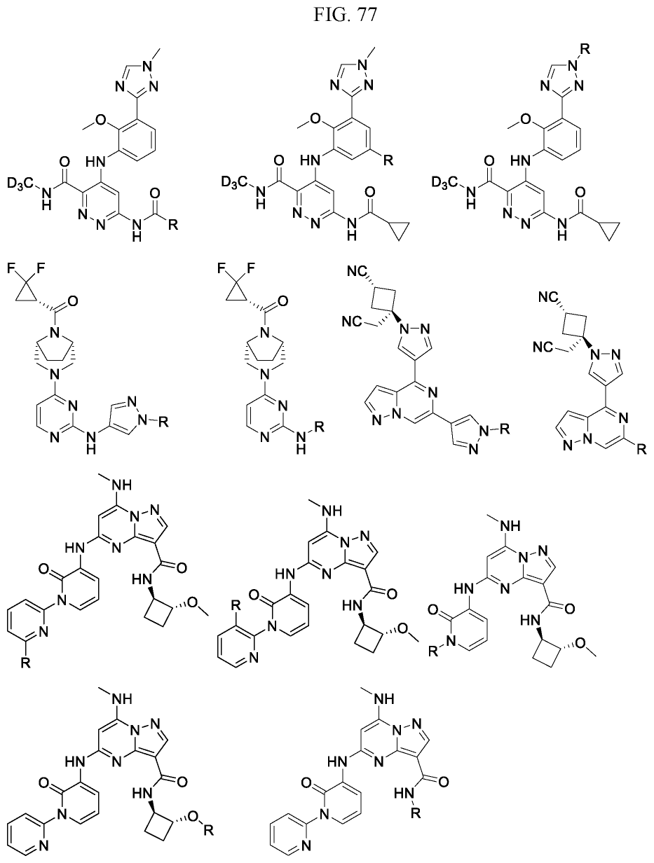

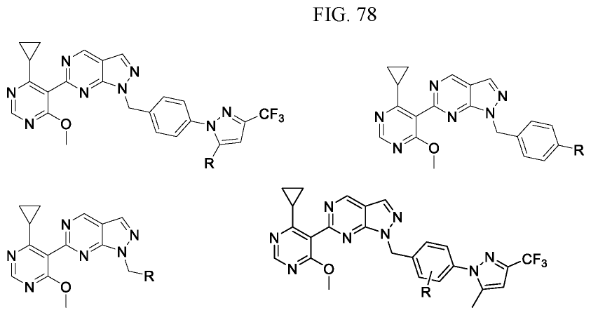

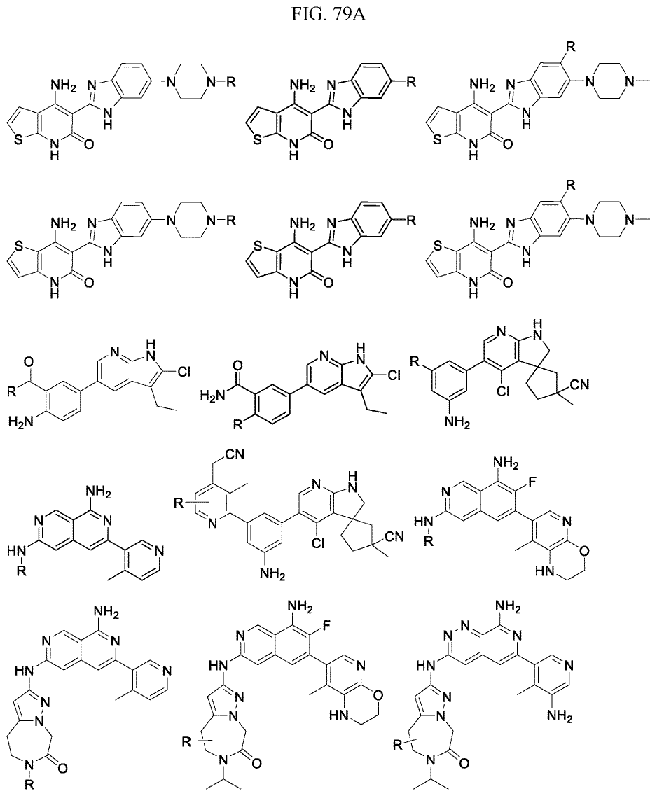

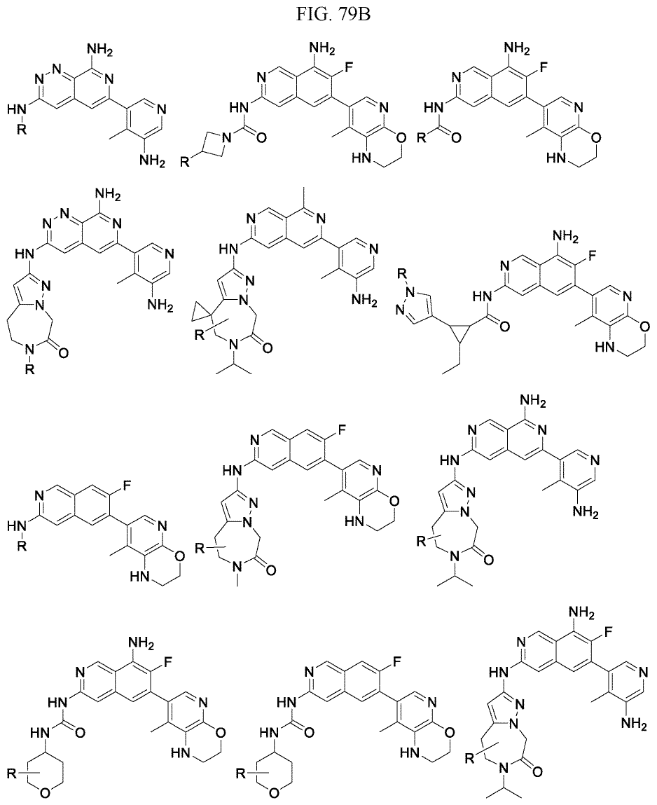

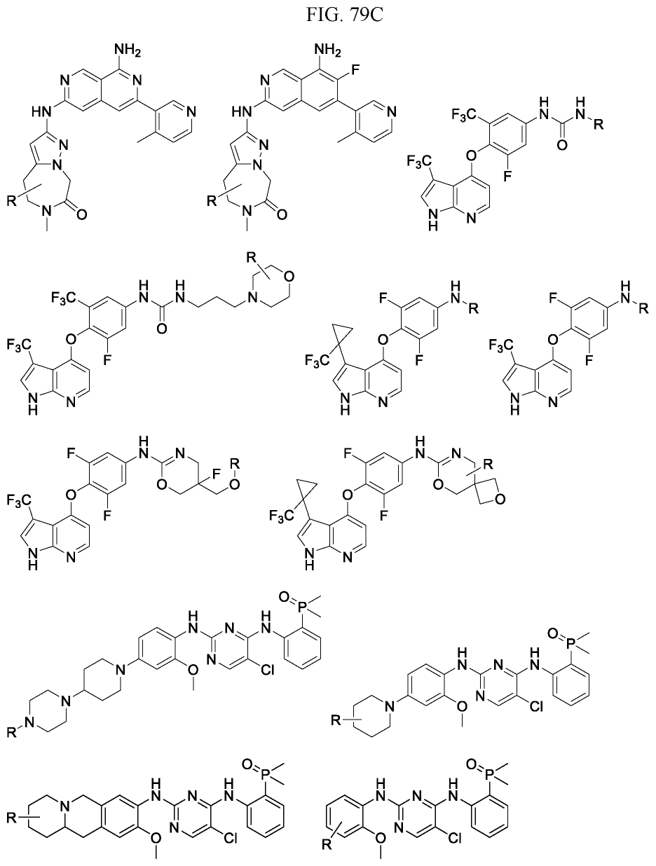

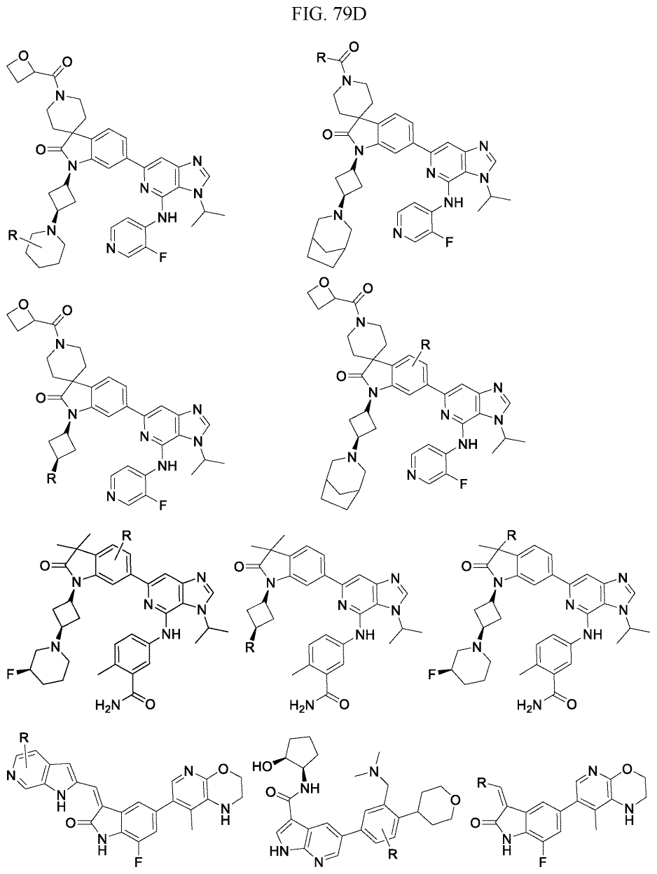

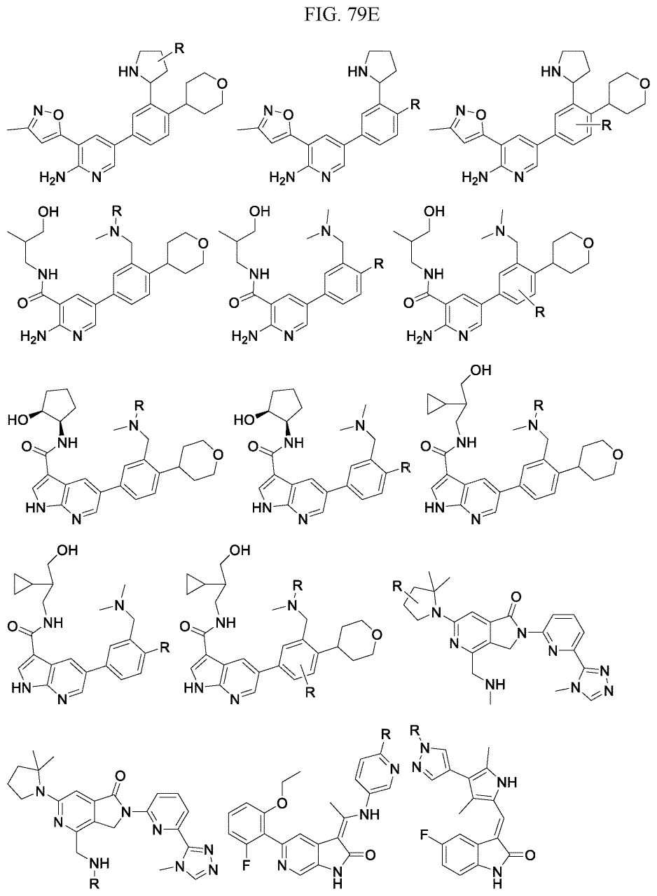

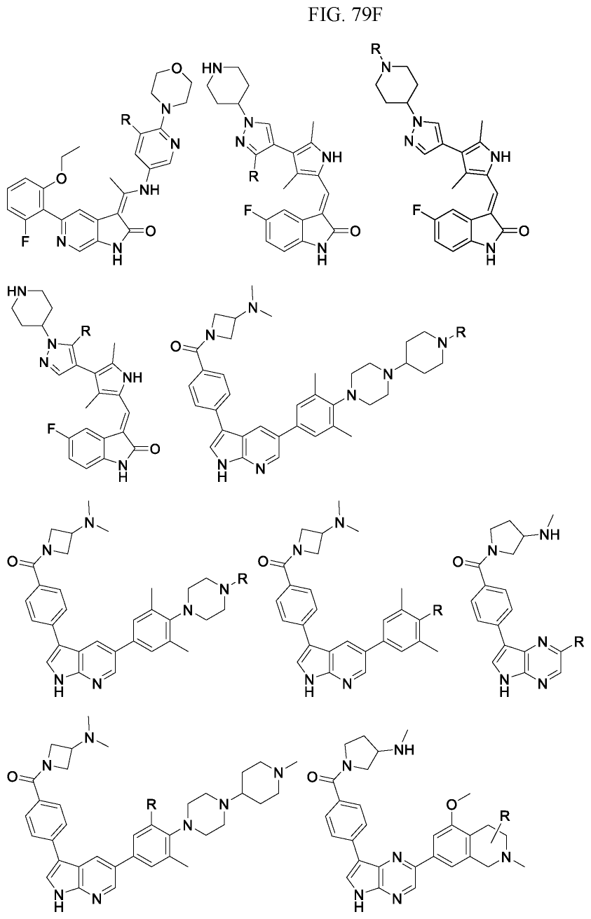

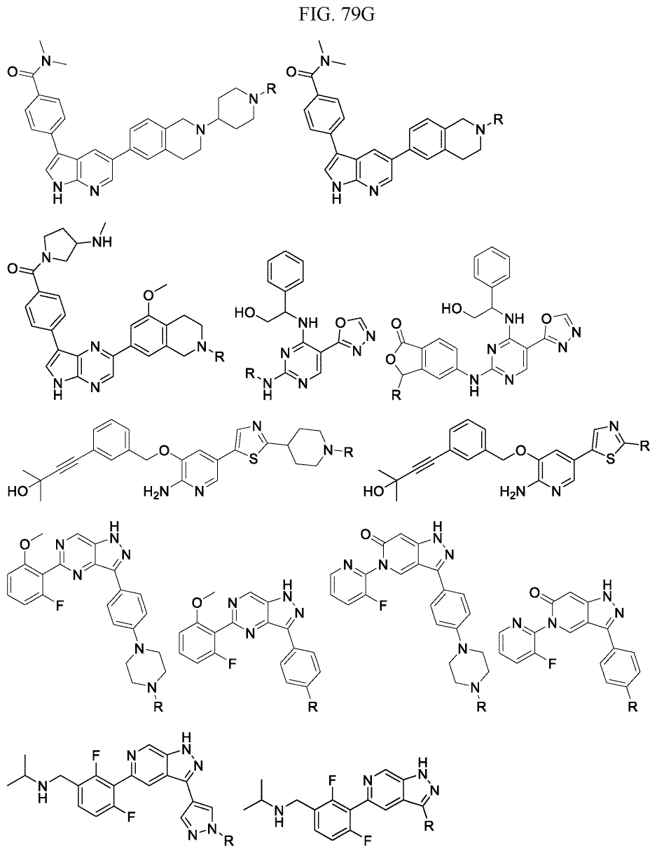

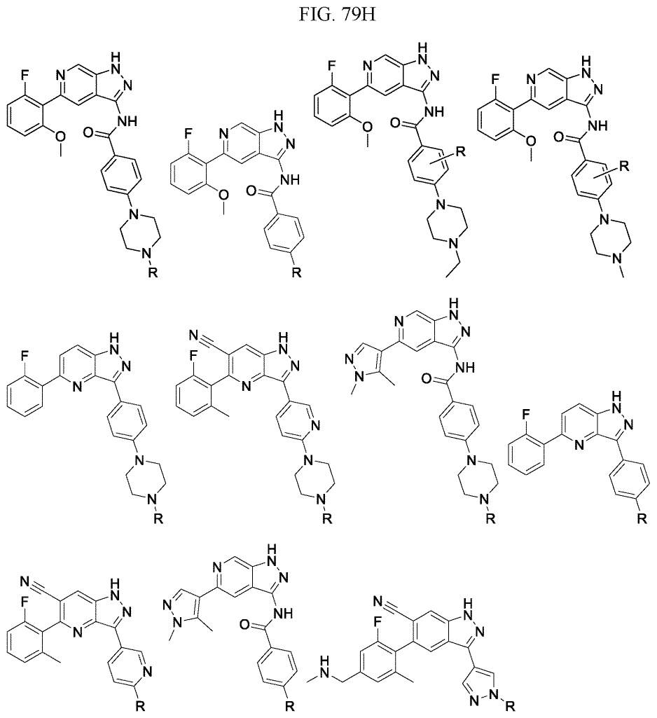

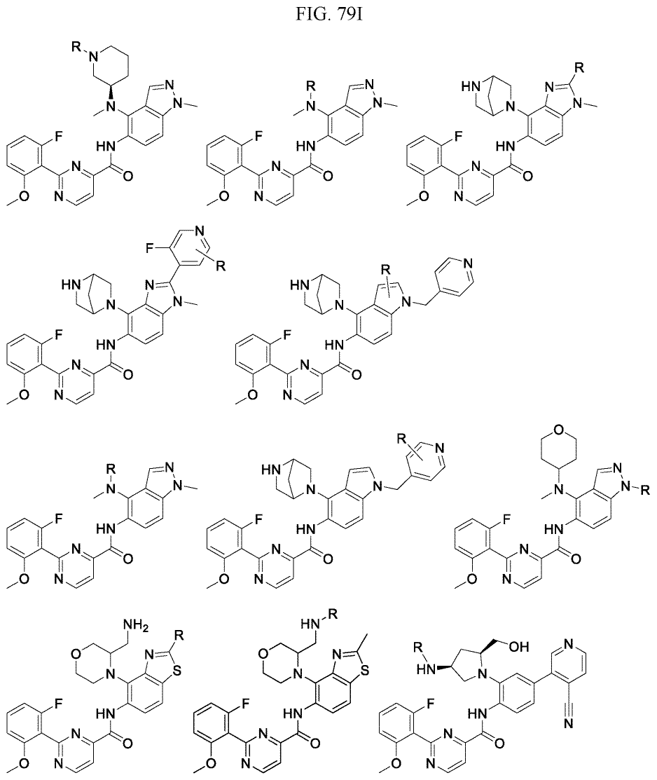

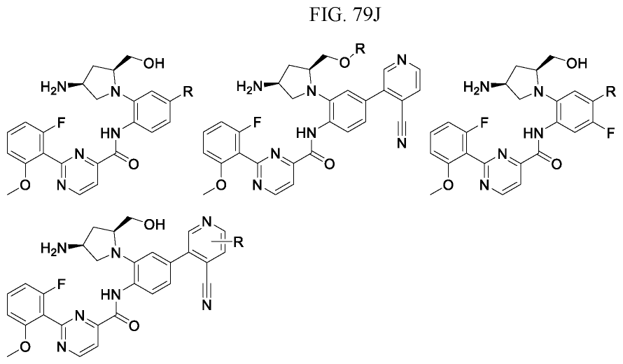

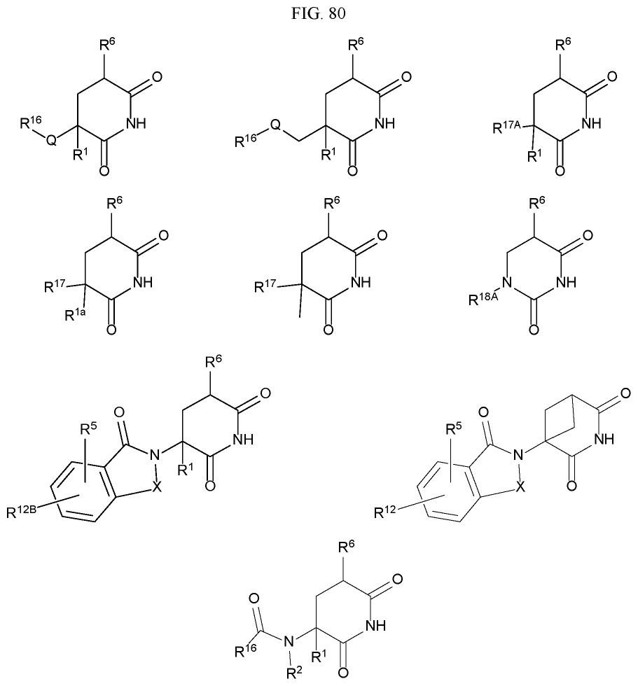

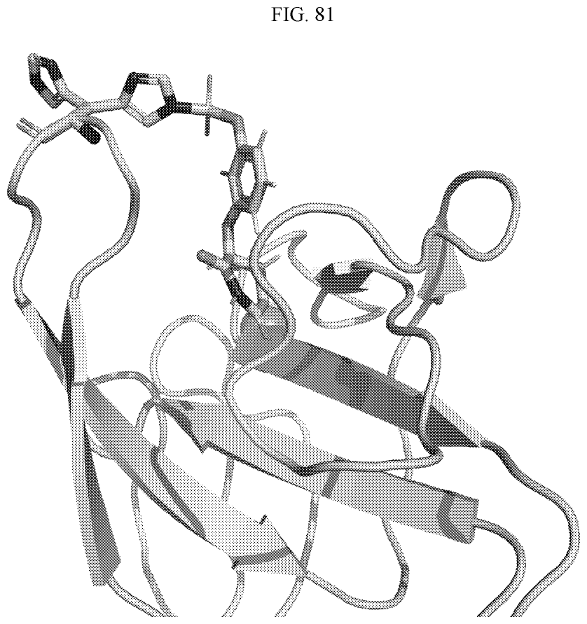

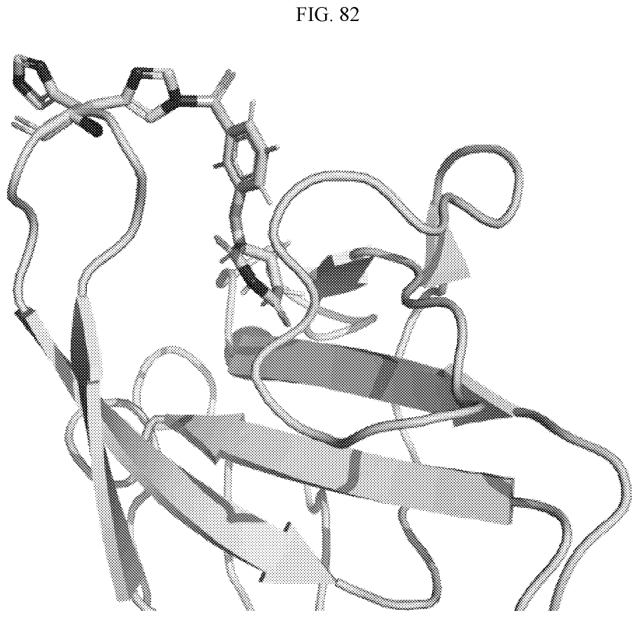

FIG. 2WW-2YY provide non-limiting examples of EGFR Targeting Ligands that target the EGFR L858R mutant, including erlotinib, gefitnib, afatinib, neratinib, and dacomitinib, wherein R represents exemplary points at which the Linker can be attached.

FIG. 2ZZ-2FFF provide non-limiting examples of EGFR Targeting Ligands that target the EGFR T790M mutant, including osimertinib, rociletinib, olmutinib, naquotinib, nazartinib, PF-06747775, Icotinib, Neratinib Avitinib, Tarloxotinib, PF-0645998, Tesevatinib, Transtinib, WZ-3146, WZ8040, and CNX-2006, wherein R represents exemplary points at which the Linker can be attached.

FIG. 2GGG provides non-limiting examples of EGFR Targeting Ligands that target the EGFR C797S mutant, including EAI045, wherein R represents exemplary points at which the Linker can be attached.

FIG. 2HHH provides non-limiting examples of BCR-ABL Targeting Ligands that target the BCR-ABL T315I mutant including Nilotinib and Dasatinib, wherein R represents exemplary points at which the Linker can be attached. See for example, the crystal structure PDB 3CS9.

FIG. 211I provides non-limiting examples of Targeting Ligands that target BCR-ABL, including Nilotinib, Dasatinib Ponatinib and Bosutinib, wherein R represents exemplary points at which the Linker can be attached.

FIG. 2JJJ-2KKK provide non-limiting examples of ALK Targeting Ligands that target the ALK L 1196M mutant including Ceritinib, wherein R represents exemplary points at which the Linker can be attached. See for example, the crystal structure PDB 4MKC.

FIG. 2LLL provides non-limiting examples of JAK2 Targeting Ligands that target the JAK2V617F mutant, including Ruxolitinib, wherein R represents exemplary points at which the Linker can be attached.

FIG. 2MMM provides non-limiting examples of BRAF Targeting Ligands that target the BRAF V600E mutant including Vemurafenib, wherein R represents exemplary points at which the Linker can be attached. For additional examples and related ligands, see, the crystal structure PBD 3OG7.

FIG. 2NNN provides non-limiting examples of BRAF Targeting Ligands, including Dabrafenib, wherein R represents exemplary points at which the Linker can be attached.

FIG. 2OOO provides non-limiting examples of LRRK2 Targeting Ligands that target the LRRK2 R1441C mutant wherein R represents exemplary points at which the Linker can be attached.

FIG. 2PPP provides non-limiting examples of LRRK2 Targeting Ligands that target the LRRK2 G2019S mutant wherein R represents exemplary points at which the Linker can be attached.

FIG. 2QQQ provides non-limiting examples of LRRK2 Targeting Ligands that target the LRRK2 I2020T mutant wherein R represents exemplary points at which the Linker can be attached.

FIG. 2RRR-2TTT provide non-limiting examples of PDGFRα Targeting Ligands that target the PDGFRα T674I mutant, including AG-1478, CHEMBL94431, Dovitinib, erlotinib, gefitinib, imatinib, Janex 1, Pazopanib, PD153035, Sorafenib, Sunitinib, and WHI-P180, wherein R represents exemplary points at which the Linker can be attached.

FIG. 2UUU provides non-limiting examples of RET Targeting Ligands that target the RET G691S mutant, including tozasertib, wherein R represents exemplary points at which the Linker can be attached.

FIG. 2VVV provides non-limiting examples of RET Targeting Ligands that target the RET R749T mutant, including tozasertib, wherein R represents exemplary points at which the Linker can be attached.

FIG. 2WWW provides non-limiting examples of RET Targeting Ligands that target the RET E762Q mutant, including tozasertib, wherein R represents exemplary points at which the Linker can be attached.

FIG. 2XXX provides non-limiting examples of RET Targeting Ligands that target the RET Y791F mutant, including tozasertib, wherein R represents exemplary points at which the Linker can be attached.

FIG. 2YYY provides non-limiting examples of RET Targeting Ligands that target the RET V804M mutant, including tozasertib, wherein R represents exemplary points at which the Linker can be attached.

FIG. 2ZZZ provides non-limiting examples of RET Targeting Ligands that target the RET M918T mutant, including tozasertib, wherein R represents exemplary points at which the Linker can be attached.

FIG. 2AAAA provides non-limiting examples of Fatty Acid Binding Protein Targeting Ligands wherein R represents exemplary points at which the Linker can be attached.

FIG. 2BBBB provides non-limiting examples of 5-Lipoxygenase Activating Protein (FLAP) Targeting Ligands wherein R represents exemplary points at which the Linker can be attached.

FIG. 2CCCC provides non-limiting examples of Kringle Domain V 4BVV Targeting Ligands wherein R represents exemplary points at which the Linker can be attached.

FIG. 2DDDD provides non-limiting examples of Lactoylglutathione Lyase Targeting Ligands wherein R represents exemplary points at which the Linker can be attached.

FIG. 2EEEE-2FFFF provide non-limiting examples of mPGES-1 Targeting Ligands wherein R represents exemplary points at which the Linker can be attached.

FIG. 2GGGG-2JJJJ provide non-limiting examples of Factor Xa Targeting Ligands wherein R represents exemplary points at which the Linker can be attached. For additional examples and related ligands, see, Maignan S. et al. “Crystal structures of human factor Xa complexed with potent inhibitors.” J. Med. Chem. 43: 3226-3232 (2000); Matsusue T. et al. 5 “Factor Xa Specific Inhibitor that Induces the Novel Binding Model in Complex with Human Fxa.” (to be published); the crystal structures PDB ligh, liqi, liqk, and ligm; Adler M. et al. “Crystal Structures of Two Potent Nonamidine Inhibitors Bound to Factor Xa.” Biochemistry 41: 15514-15523 (2002); Roehrig S. et al. “Discovery of the Novel Antithrombotic Agent 5-Chloro-N-({(5S)-2-Oxo-3-[4-(3-Oxomorpholin-4-Yl)Phenyl]-1 3-Oxazolidin-5-Yl}Methyl)Thiophene-2- Carboxamide (Bay 59-7939): An Oral Direct Factor Xa Inhibitor.” J. Med. Chem. 48: 5900 (2005); Anselm L. et al. “Discovery of a Factor Xa Inhibitor (3R 4R)-1-(2 2-Difluoro-Ethyl)-Pyrrolidine-3 4-Dicarboxylic Acid 3-[(5-Chloro-Pyridin-2-Yl)-Amide]4-{[2-Fluoro-4-(2-Oxo-2H-Pyridin-1-Yl)-Phenyl]-Amide}as a Clinical Candidate.” Bioorg. Med. Chem. 20: 5313 (2010); and, Pinto D.J. et al. “Discovery of 1-(4-Methoxyphenyl)-7-oxo-6-(4-(2-oxopiperidin-1-yl)phenyl)-4 5 6 7-tetrahydro-1H-pyrazolo[3 4-c]pyridine-3-carboxamide (Apixaban BMS-562247) a Highly Potent Selective Efficacious and Orally Bioavailable Inhibitor of Blood Coagulation Factor Xa.” J. Med. Chem. 50: 5339-5356 (2007).

FIG. 2KKKK provides non-limiting examples of Kallikrein 7 Targeting Ligands wherein R represents exemplary points at which the Linker can be attached. For additional examples and related ligands, see, Maibaum J. et al. “Small-molecule factor D inhibitors targeting the alternative complement pathway.” Nat. Chem. Biol. 12: 1105-1110 (2016).

FIG. 2LLLL-2MMMM provide non-limiting examples of Cathepsin K Targeting Ligands wherein R represents exemplary points at which the Linker can be attached. For additional examples and related ligands, see, Rankovic Z. et al. “Design and optimization of a series of novel 2-cyano-pyrimidines as cathepsin K inhibitors” Bioorg. Med. Chem. Lett. 20: 1524-1527 (2010); and, Cai J. et al. “Trifluoromethylphenyl as P2 for ketoamide-based cathepsin S inhibitors.” Bioorg. Med. Chem. Lett. 20: 6890-6894 (2010).

FIG. 2NNNN provides non-limiting examples of Cathepsin L Targeting Ligands wherein R represents exemplary points at which the Linker can be attached. For additional examples and related ligands, see, Kuhn B. et al. “Prospective Evaluation of Free Energy Calculations for the Prioritization of Cathepsin L Inhibitors.” J Med. Chem. 60: 2485-2497 (2017).

FIG. 2OOOO provides non-limiting examples of Cathepsin S Targeting Ligands wherein R represents exemplary points at which the Linker can be attached. For additional examples and related ligands, see, Jadhav P.K. et al. “Discovery of Cathepsin S Inhibitor LY3000328 for the Treatment of Abdominal Aortic Aneurysm” ACS Med Chem. Lett. 5: 1138-1142.” (2014).

FIG. 2PPPP-2SSSS provide non-limiting examples of MTH1 Targeting Ligands wherein R represents exemplary points at which the Linker can be attached. For additional examples and related ligands, see, Kettle J.G. et al. “Potent and Selective Inhibitors of Mthl Probe its Role in Cancer Cell Survival.” J. Med Chem. 59: 2346 (2016); Huber K.V.M. et al. “Stereospecific Targeting of Mthl by (S)-Crizotinib as an Anticancer Strategy.” Nature 508: 222 (2014); Gad H. et al. “MTH1 inhibition eradicates cancer by preventing sanitation of the dNTP pool.” Nature 508: 215-221 (2014); Nissink J.W.M. et al. “Mthl Substrate Recognition--an Example of Specific Promiscuity.” Plos One 11: 51154 (2016); and, Manuel Ellermann et al. “Novel class of potent and selective inhibitors efface MTH1 as broad-spectrum cancer target.” AACR National Meeting Abstract 5226, 2017.

FIG. 2TTTT-2ZZZZ provide non-limiting examples of MDM2 and/or MDM4 Targeting Ligands wherein R represents exemplary points at which the Linker can be attached. For additional examples and related ligands, see, Popowicz G.M. et al. “Structures of low molecular weight inhibitors bound to MDMX and MDM2 reveal new approaches for p53-MDMX/MDM2 antagonist drug discovery.” Cell Cycle, 9 (2010); Miyazaki M. et al. “Synthesis and evaluation of novel orally active p53-MDM2 interaction inhibitors.” Bioorg. Med Chem. 21: 4319-4331 (2013); Miyazaki M. et al. “Discovery of DS-5272 as a promising candidate: A potent and orally active p53-MDM2 interaction inhibitor.” Bioorg Med Chem. 23: 2360-7 (2015); Holzer P. et al. “Discovery of a Dihydroisoquinolinone Derivative (NVP-CGM097): A Highly Potent and Selective MDM2 Inhibitor Undergoing Phase 1 Clinical Trials in p53 wt Tumors.” J. Med Chem. 58: 6348-6358 (2015); Gonzalez-Lopez de Turiso F. et al. “Rational Design and Binding Mode Duality of MDM2-p53 Inhibitors.” J. Med. Chem. 56: 4053-4070 (2013); Gessier F. et al. “Discovery of dihydroisoquinolinone derivatives as novel inhibitors of the p53-MDM2 interaction with a distinct binding mode.” Bioorg. Med Chem. Lett. 25: 3621-3625 (2015); Fry D.C. et al. “Deconstruction of a nutlin: dissecting the binding determinants of a potent protein-protein interaction inhibitor.” ACS Med Chem Lett 4: 660-665 (2013); Ding Q. et al. “Discovery of RG7388 a Potent and Selective p53-MDM2 Inhibitor in Clinical Development.” J. Med Chem. 56: 5979-5983 (2013); Wang S. et al. “SAR405838: an optimized inhibitor of MDM2-p53 interaction that induces complete and durable tumor regression.” Cancer Res. 74: 5855-5865 (2014); Rew Y. et al. “Discovery of AM-7209 a Potent and Selective 4-Amidobenzoic Acid Inhibitor of the MDM2-p53 Interaction.” J. Med. Chem. 57: 10499-10511 (2014); Bogen S.L. et al. “Discovery of Novel 3 3-Disubstituted Piperidines as Orally Bioavailable Potent and Efficacious HDM2-p53 Inhibitors.” ACSMed. Chem. Lett. 7: 324-329 (2016); and, Sun D. et al. “Discovery of AMG 232 a Potent Selective and Orally Bioavailable MDM2-p53 Inhibitor in Clinical Development.” J. Med. Chem. 57: 1454-1472 (2014).

FIG. 2AAAAA-2EEEEE provide non-limiting examples of PARP1, PARP2, and/or PARP3 Targeting Ligands wherein R represents exemplary points at which the Linker can be attached. For additional examples and related ligands, see, Iwashita A. et al. “Discovery of quinazolinone and quinoxaline derivatives as potent and selective poly(ADP-ribose) polymerase-1/2 inhibitors.” Febs Lett. 579: 1389-1393 (2005); the crystal structure PDB 2RCW (PARP complexed with A861695, Park C.H.); the crystal structure PDB 2RD6 (PARP complexed with A861696, Park C.H.); the crystal structure PDB 3GN7; Miyashiro J. et al. “Synthesis and SAR of novel tricyclic quinoxalinone inhibitors of poly(ADP-ribose)polymerase-1 (PARP-1)” Bioorg. Med. Chem. Lett. 19: 4050-4054 (2009); Gandhi V.B. et al. “Discovery and SAR of substituted 3-oxoisoindoline-4-carboxamides as potent inhibitors of poly(ADP-ribose) polymerase (PARP) for the treatment of cancer.” Bioorg. Med. Chem. Lett. 20: 1023-1026 (2010); Penning T.D. et al. “Optimization of phenyl-substituted benzimidazole carboxamide poly(ADP-ribose) polymerase inhibitors: identification of (S)-2-(2-fluoro-4-(pyrrolidin-2-yl)phenyl)-1H-benzimidazole-4-carboxamide (A-966492) a highly potent and efficacious inhibitor.” J Med. Chem. 53: 3142-3153 (2010); Ye N. et al. “Design, Synthesis, and Biological Evaluation of a Series of Benzo[de][1 7]naphthyridin-7(8H)-ones Bearing a Functionalized Longer Chain Appendage as Novel PARP1 Inhibitors.” J. Med. Chem. 56: 2885-2903 (2013); Patel M.R. et al. “Discovery and Structure-Activity Relationship of Novel 2 3-Dihydrobenzofuran-7-carboxamide and 2 3-Dihydrobenzofuran-3(2H)-one-7-carboxamide Derivatives as Poly(ADP-ribose)polymerase-1 Inhibitors.” J. Med. Chem. 57: 5579-5601 (2014); Thorsell A.G. et al. “Structural Basis for Potency and Promiscuity in Poly(ADP-ribose) Polymerase (PARP) and Tankyrase Inhibitors. “J. Med. Chem. 60:1262-1271 (2012); the crystal structure PDB 4RV6 (“Human ARTD1 (PARP1) catalytic domain in complex with inhibitor Rucaparib”, Karlberg T. et al.); Papeo G.M.E. et al. “Discovery of 2-[1-(4 4-Difluorocyclohexyl)Piperidin-4-Yl]-6-Fluoro-3-Oxo-2 3-Dihydro-1H-Isoindole-4-Carboxamide (Nms-P118): A Potent Orally Available and Highly Selective Parp- 1 Inhibitor for Cancer Therapy.” J. Med. Chem. 58: 6875 (2015); Kinoshita T. et al. “Inhibitor-induced structural change of the active site of human poly(ADP-ribose) polymerase.” Febs Lett. 556: 43-46 (2004); and, Gangloff A.R. et al. “Discovery of novel benzo[b][1 4]oxazin-3(4H)-ones as poly(ADP-ribose)polymerase inhibitors.” Bioorg. Med. Chem. Lett. 23: 4501-4505 (2013).

FIG. 2FFFFF-2GGGGG provide non-limiting examples of PARP14 Targeting Ligands wherein R represents exemplary points at which the Linker can be attached.

FIG. 2HHHHH provides non-limiting examples of PARP15 Targeting Ligands wherein R represents exemplary points at which the Linker can be attached.

FIG. 211111 provides non-limiting examples of PDZ domain Targeting Ligands wherein R represents exemplary points at which the spacer(s) are attached.

FIG. 2JJJJJ provides non-limiting examples of Phospholipase A2 domain Targeting Ligands wherein R represents exemplary points at which the Linker can be attached.

FIG. 2KKKKK provides non-limiting examples of Protein S100-A7 2WOS Targeting Ligands wherein R represents exemplary points at which the Linker can be attached.

FIG. 2LLLLL-2MMMMM provide non-limiting examples of Saposin-B Targeting Ligands wherein R represents exemplary points at which the Linker can be attached.

FIG. 2NNNNN-2OOOOO provide non-limiting examples of Sec7 Targeting Ligands wherein R represents exemplary points at which the Linker can be attached.

FIG. 2PPPPP-2QQQQQ provide non-limiting examples of SH2 domain of pp60 Src Targeting Ligands wherein R represents exemplary points at which the Linker can be attached.

FIG. 2RRRRR provides non-limiting examples of Tank1 Targeting Ligands wherein R represents exemplary points at which the Linker can be attached.

FIG. 2SSSSS provides non-limiting examples of Ubc9 SUMO E2 ligase SF6D Targeting Ligands wherein R represents exemplary points at which the Linker can be attached.

FIG. 2TTTTT provides non-limiting examples of Src Targenting Ligands, including AP23464, wherein R represents exemplary points at which the Linker can be attached.

FIG. 2UUUUU-2XXXXX provide non-limiting examples of Src-AS1 and/or Src AS2 Targeting Ligands wherein R represents exemplary points at which the Linker can be attached.

FIG. 2YYYYY provides non-limiting examples of JAK3 Targeting Ligands, including Tofacitinib, wherein R represents exemplary points at which the Linker can be attached.

FIG. 2ZZZZZ provides non-limiting examples of ABL Targeting Ligands, including Tofacitinib and Ponatinib, wherein R represents exemplary points at which the Linker can be attached.

FIG. 3A-3B provide non-limiting examples of MEKI Targeting Ligands, including PD318088, Trametinib and G-573, wherein R represents exemplary points at which the Linker can be attached.

FIG. 3C provides non-limiting examples of KIT Targeting Ligands, including Regorafenib, wherein R represents exemplary points at which the Linker can be attached.

FIG. 3D-3E provide non-limiting examples of HIV Reverse Transcriptase Targeting Ligands, including Efavirenz, Tenofovir, Emtricitabine, Ritonavir, Raltegravir, and Atazanavir, wherein R represents exemplary points at which the Linker can be attached.

FIG. 3F-3G provide non-limiting examples of HIV Protease Targeting Ligands, including Ritonavir, Raltegravir, and Atazanavir, wherein R represents exemplary points at which the Linker can be attached.

FIG. 311-3I provide non-limiting examples of KSR1 Targeting Ligands wherein R represents exemplary points at which the Linker can be attached.

FIG. 3J-3L provide non-limiting examples of CTNNB1 Targeting Ligands wherein R represents exemplary points at which the Linker can be attached. (See “Direct Targeting of b-Catenin by a Small Molecule Stimulates Proteasomal Degradation and Suppresses Oncogenic Wnt/b-Catenin Signaling” Cell Rep 2016, 16(1), 28; “Rational Design of Small-Molecule Inhibitors for β-Catenin/T-Cell Factor Protein-Protein Interactions by Bioisostere Replacement” ACS Chem Biol 2013, 8, 524; and “Allosteric inhibitor of β-catenin selectively targets oncogenic Wnt signaling in colon cancer” Sci Rep 2020, 10, 8096).

FIG. 3M provides non-limiting examples of BCL6 Targeting Ligands wherein R represents exemplary points at which the Linker can be attached.

FIG. 3N-30 provide non-limiting examples of PAK1 Targeting Ligands wherein R represents exemplary points at which the Linker can be attached.

FIG. 3P-3R provide non-limiting examples of PAK4 Targeting Ligands wherein R represents exemplary points at which the Linker can be attached.

FIG. 3S-3T provide non-limiting examples of TNIK Targeting Ligands wherein R represents exemplary points at which the Linker can be attached.

FIG. 3U provides non-limiting examples of MENI Targeting Ligands wherein R represents exemplary points at which the Linker can be attached.

FIG. 3V-3W provide non-limiting examples of ERKI Targeting Ligands wherein R represents exemplary points at which the Linker can be attached.

FIG. 3X provides non-limiting examples of IDO1 Targeting Ligands wherein R represents exemplary points at which the Linker can be attached.

FIG. 3Y provides non-limiting examples of CBP Targeting Ligands wherein R represents exemplary points at which the Linker can be attached.

FIG. 3Z-3SS provide non-limiting examples of MCL1 Targeting Ligands wherein R represents exemplary points at which the Linker can be attached. For additional examples and related ligands, see, Tanaka Y._et al “Discovery of potent Mcl-1/Bcl-xL dual inhibitors by using a hybridization strategy based on structural analysis of target proteins.” J. Med. Chem. 56: 9635-9645 (2013); Friberg A._et al. “Discovery of potent myeloid cell leukemia 1 (Mcl-1) inhibitors using fragment-based methods and structure-based design.” J. Med Chem. 56: 15-30 (2013); Petros A. M. et al “Fragment-based discovery of potent inhibitors of the anti-apoptotic MCL-1 protein.” Bioorg. Med Chem. Lett. 24: 1484-1488 (2014); Burke J.P. et al. “Discovery of tricyclic indoles that potently inhibit mcl-1 using fragment-based methods and structure-based design.” J. Med. Chem. 58: 3794-3805 (2015); Pelz N.F. et al. “Discovery of 2-Indole-acylsulfonamide Myeloid Cell Leukemia 1 (Mcl-1) Inhibitors Using Fragment-Based Methods.” J. Med Chem. 59: 2054-2066 (2016); Clifton M.C. et al. “A Maltose-Binding Protein Fusion Construct Yields a Robust Crystallography Platform for MCL1.” Plos One 10: e0125010-e0125010 (2015); Kotschy A et al. “The MCL1 inhibitor S63845 is tolerable and effective in diverse cancer models. Nature 538:477-482 (2016); EP 2886545 A1 titled “New thienopyrimidine derivatives a process for their preparation and pharmaceutical compositions containing them”; Jeffrey W. Johannes et al. “Structure Based Design of Non-Natural Peptidic Macrocyclic Mcl-1 Inhibitors” ACS Med Chem. Lett. (2017); DOI: 10.1021/acsmedchemlett.6b00464; Bruncko M. et al. “Structure-Guided Design of a Series of MCL-1 Inhibitors with High Affinity and Selectivity.” J Med Chem. 58: 2180-2194 (2015); Taekyu Lee et al. “Discovery and biological characterization of potent myeloid cell leukemia-1 inhibitors.” FEBSLetters 591: 240-251 (2017); Chen L.et al. “Structure-Based Design of 3-Carboxy-Substituted 1 2 3 4- Tetrahydroquinolines as Inhibitors of Myeloid Cell Leukemia-1 (Mcl-1).” Org. Biomol. Chem. 14:5505-5510 (2016); US 2016/0068545 titled “Tetrahydronaphthalene derivatives that inhibit mcl-1 protein”; WO 2016207217 A1 titled “Preparation of new bicyclic derivatives as pro-apoptotic agents”; Gizem Akgay et al. “Inhibition of Mcl-1 through covalent modification of a noncatalytic lysine side chain” Nature Chemical Biology 12: 931-936 (2016).

FIG. 3TT provides non-limiting examples of ASHIL Targeting Ligands wherein R represents exemplary points at which the Linker can be attached. See for example, the crystal structure PDB 4YNM (“Human ASHIL SET domain in complex with S-adenosyl methionine (SAM)” Rogawski D.S. et al.)

FIG. 3UU-3WW provide non-limiting examples of ATAD2 Targeting Ligands wherein R represents exemplary points at which the Linker can be attached. For additional examples and related ligands, see, Chaikuad A. et al. “Structure-based approaches towards identification of fragments for the low-drugability ATAD2 bromodomain” Med Chem Comm 5: 1843-1848 (2014); Poncet-Montange G. et al. “Observed bromodomain flexibility reveals histone peptide- and small molecule ligand-compatible forms of ATAD2.” Biochem. J. 466: 337-346 (2015); Harner M.J. et al. “Fragment-Based Screening of the Bromodomain of ATAD2.” J. Med. Chem. 57: 9687-9692 (2014); Demont E.H. et al. “Fragment-Based Discovery of Low-Micromolar Atad2 Bromodomain Inhibitors.” J. Med. Chem. 58: 5649 (2015); and, Bamborough P. et al. “Structure-Based Optimization of Naphthyridones into Potent Atad2 Bromodomain Inhibitors.” J. Med. Chem. 58: 6151 (2015).

FIG. 3XX-3AAA provide non-limiting examples of BAZ2A and BAZ2B Targeting Ligands wherein R represents exemplary points at which the Linker can be attached. For additional examples and related ligands, see, the crystal structure PDB 4CUU (“Human Baz2B in Complex with Fragment-6 N09645” Bradley A. et al.); the crystal structure PDB 5CUA (“Second Bromodomain of Bromodomain Adjacent to Zinc Finger Domain Protein 2B (BAZ2B) in complex with 1-Acetyl-4-(4-hydroxyphenyl)piperazine”. Bradley A. et al.); Ferguson F.M. et al. “Targeting low-drugability bromodomains: fragment based screening and inhibitor design against the BAZ2B bromodomain.” J. Med. Chem. 56: 10183-10187 (2013); Marchand J.R. et al. “Derivatives of 3-Amino-2-methylpyridine as BAZ2B Bromodomain Ligands: In Silico Discovery and in Crystallo Validation.” J. Med. Chem. 59: 9919-9927 (2016); Drouin L. et al. “Structure Enabled Design of BAZ2-ICR A Chemical Probe Targeting the Bromodomains of BAZ2A and BAZ2B.” J. Med. Chem. 58: 2553-2559 (2015); Chen P. et al. “Discovery and characterization of GSK2801 a selective chemical probe for the bromodomains BAZ2A and BAZ2B.” J Med. Chem. 59:1410-1424 (2016).

FIG. 3BBB provides non-limiting examples of BRD1 Targeting Ligands wherein R represents exemplary points at which the Linker can be attached. For additional examples and related ligands, see, the crystal structure PDB 5AME (“the Crystal Structure of the Bromodomain of Human Surface Epitope Engineered Brd1A in Complex with 3D Consortium Fragment 4-Acetyl-Piperazin-2-One Pearce”, N.M. et al.); the crystal structure PDB 5AMF (“Crystal Structure of the Bromodomain of Human Surface Epitope Engineered Brd1A in Complex with 3D Consortium Fragment Ethyl 4 5 6 7-Tetrahydro-1H-Indazole-5-Carboxylate”, Pearce N.M. et al.); the crystal structure PDB 5FG6 (“the Crystal structure of the bromodomain of human BRD1 (BRPF2) in complex with OF-1 chemical probe.”, Tallant C. et al.); Filippakopoulos P. et al. “Histone recognition and large-scale structural analysis of the human bromodomain family.” Cell, 149: 214-231 (2012).

FIG. 3CCC-3EEE provide non-limiting examples of BRD2 Bromodomain 1 Targeting Ligands wherein R represents exemplary points at which the Linker can be attached. For additional examples and related ligands, see, the crystal structure PDB 2ydw; the crystal structure PDB 2yek; the crystal structure PDB 4a9h; the crystal structure PDB 4a9f; the crystal structure PDB 4a9i; the crystal structure PDB 4a9m; the crystal structure PDB 4akn; the crystal structure PDB 4alg, and the crystal structure PDB 4uyf.

FIG. 3FFF-3HHH provide non-limiting examples of BRD2 Bromodomain 2 Targeting Ligands wherein R represents exemplary points at which the Linker can be attached. For additional examples and related ligands, see, the crystal structure PDB 3oni; Filippakopoulos P. et al. “Selective Inhibition of BET Bromodomains.” Nature 468: 1067-1073 (2010); the crystal structure PDB 4jlp; McLure K.G. et al. “RVX-208: an Inducer of ApoA-I in Humans is a BET Bromodomain Antagonist.” Plos One 8: e83190-e83190 (2013); Baud M.G. et al. “Chemical biology. A bump-and-hole approach to engineer controlled selectivity of BET bromodomain chemical probes” Science 346: 638-641 (2014); Baud M.G. et al. “New Synthetic Routes to Triazolo-benzodiazepine Analogues: Expanding the Scope of the Bump-and-Hole Approach for Selective Bromo and Extra-Terminal (BET) Bromodomain Inhibition” J. Med. Chem. 59: 1492-1500 (2016); Gosmini R. et al. “The Discovery of I-Bet726 (Gsk1324726A) a Potent Tetrahydroquinoline Apoal Up-Regulator and Selective Bet Bromodomain Inhibitor” J. Med. Chem. 57: 8111 (2014); the crystal structure PDB 5EK9 (“Crystal structure of the second bromodomain of human BRD2 in complex with a hydroquinolinone inhibitor”, Tallant C. et al); the crystal structure PDB 5BT5; the crystal structure PDB 5dfd; Baud M.G. et al. “New Synthetic Routes to Triazolo-benzodiazepine Analogues: Expanding the Scope of the Bump-and-Hole Approach for Selective Bromo and Extra-Terminal (BET) Bromodomain Inhibition” J. Med. Chem. 59: 1492-1500 (2016).

FIG. 3III-3JJJ provide non-limiting examples of BRD4 Bromodomain 1 Targeting Ligands wherein R represents exemplary points at which the Linker can be attached. For additional examples and related ligands, see, the crystal structure PDB 5WUU and the crystal structure PDB 5F5Z.

FIG. 3KKK-3LLL provide non-limiting examples of BRD4 Bromodomain 2 Targeting Ligands wherein R represents exemplary points at which the Linker can be attached. For additional examples and related ligands, see, Chung C.W. et al. “Discovery and Characterization of Small Molecule Inhibitors of the Bet Family Bromodomains” J. Med. Chem. 54: 3827 (2011) and Ran X. et al. “Structure-Based Design of gamma-Carboline Analogues as Potent and Specific BET Bromodomain Inhibitors” J. Med. Chem. 58: 4927-4939 (2015).

FIG. 3MMM provides non-limiting examples of BRDT Targeting Ligands wherein R represents exemplary points at which the Linker can be attached. For additional examples and related ligands, see, the crystal structure PDB 4flp and the crystal structure PDB 4kcx.

FIG. 3NNN-3QQQ provide non-limiting examples of BRD9 Targeting Ligands wherein R represents exemplary points at which the Linker can be attached. For additional examples and related ligands, see, the crystal structure PDB 4ngn; the crystal structure PDB 4uit; the crystal structure PDB 4uiu; the crystal structure PDB 4uiv; the crystal structure PDB 4z6h; the crystal structure PDB 4z6i; the crystal structure PDB 5e9v; the crystal structure PDB 5eul; the crystal structure PDB 5flh; the crystal structure PDB 5fp2, (“Structure-Based Design of an in Vivo Active Selective BRD9 Inhibitor” J Med Chem., 2016, 59(10), 4462; and WO2016139361).

FIG. 3RRR provides non-limiting examples of SMARCA4 PB1 and/or SMARCA2 Targeting Ligands wherein R represents exemplary points at which the Linker can be attached, A is N or CH, and m is 0 12 3 4 5 6 7 or 8.

FIG. 3SSS-3XXX provide non-limiting examples of additional Bromodomain Targeting Ligands wherein R represents exemplary points at which the Linker can be attached. For additional examples and related ligands, see, Hewings et al. “3 5-Dimethylisoxazoles Act as Acetyl-lysine Bromodomain Ligands.” J. Med. Chem. 54 6761-6770 (2011); Dawson et al. “Inhibition of BET Recruitment to Chromatin as an Effective Treatment for MLL-fusion Leukemia.” Nature, 478, 529-533 (2011); US 2015/0256700; US 2015/0148342; WO 2015/074064; WO 2015/067770; WO 2015/022332; WO 2015/015318; and, WO 2015/011084.

FIG. 3YYY provides non-limiting examples of PB1 Targeting Ligands wherein R represents exemplary points at which the Linker can be attached. For additional examples and related ligands, see, the crystal structure PDB 3mb4; the crystal structure PDB 4qOn; and, the crystal structure PDB 5fh6.

FIG. 3ZZZ provides non-limiting examples of SMARCA4 Targeting Ligands wherein R represents exemplary points at which the Linker can be attached. For additional examples and related ligands, see, the crystal structure 3uvd and the crystal structure 5dkd.

FIG. 3AAAA provides non-limiting examples of SMARCA2 Targeting Ligands wherein R represents exemplary points at which the Linker can be attached. For additional examples and related ligands, see, the crystal structure 5dkc and the crystal structure 5dkh; and WO2020023657, US20200038378, WO2020010227, WO2020078933, WO2019207538, WO2016138114, WO2020035779, and “Discovery of Orally Active Inhibitors of Brahma Homolog (BRM)/SMARCA2 ATPase Activity for the Treatment of Brahma Related Gene 1 (BRG1)/SMARCA4-Mutant Cancers” J Med Chem 2018, 61, 10155.

FIG. 3BBBB provides non-limiting examples of TRIM24 (TIF1a) and/or BRPF1 Targeting Ligands wherein R represents exemplary points at which the Linker can be attached and m is 0 1 2 3 4 5 6 7 or 8.

FIG. 3CCCC provides non-limiting examples of TRIM24 (TIIF Ia) Targeting Ligands wherein R represents exemplary points at which the Linker can be attached. For additional examples and related ligands, see, Palmer W.S. et al. “Structure-Guided Design of IACS-9571: a Selective High-Affinity Dual TRIM24-BRPF1 Bromodomain Inhibitor.” J. Med. Chem. 59: 1440-1454 (2016).

FIG. 3DDDD-3FFFF provide non-limiting examples of BRPF1 Targeting Ligands wherein R represents exemplary points at which the Linker can be attached. For additional examples and related ligands, see, the crystal structure PDB 4uye; the crystal structure PDB 5c7n; the crystal structure PDB 5c87; the crystal structure PDB 5c89; the crystal structure PDB 5d7x; the crystal structure PDB 5dya; the crystal structure PDB 5epr; the crystal structure PDB 5eql; the crystal structure PDB 5etb; the crystal structure PDB 5ev9; the crystal structure PDB 5eva; the crystal structure PDB 5ewv; the crystal structure PDB 5eww; the crystal structure PDB 5ffy; the crystal structure PDB 5fg5; and, the crystal structure PDB 5g4r.

FIG. 3GGGG provides non-limiting examples of CECR2 Targeting Ligands wherein R represents exemplary points at which the Linker can be attached. For additional examples and related ligands, see, Moustakim M. et al. Med. Chem. Comm. 7:2246-2264 (2016) and Crawford T. et al. Journal ofMed. Chem. 59; 5391-5402 (2016).

FIG. 3HHHH-30000 provide non-limiting examples of CREBBP Targeting Ligands wherein R represents exemplary points at which the Linker can be attached, A is N or CH, and m is 0 1 2 3 4 5 6 7 or 8. For additional examples and related ligands, see, the crystal structure PDB 3pld; the crystal structure PDB 3svh; the crystal structure PDB 4nr4; the crystal structure PDB 4nr5; the crystal structure PDB 4ts8; the crystal structure PDB 4nr6; the crystal structure PDB 4nr7; the crystal structure PDB 4nyw; the crystal structure PDB 4nyx; the crystal structure PDB 4tqn; the crystal structure PDB 5cgp; the crystal structure PDB 5dbm; the crystal structure PDB 5ep7; the crystal structure PDB 5i83; the crystal structure PDB 5i86; the crystal structure PDB 5i89; the crystal structure PDB 5i8g; the crystal structure PDB 5jOd; the crystal structure PDB 5ktu; the crystal structure PDB 5ktw; the crystal structure PDB 5ktx; the crystal structure PDB 5tb6.

FIG. 3PPPP provides non-limiting examples of EP300 Targeting Ligands wherein R represents exemplary points at which the Linker can be attached. For additional examples and related ligands, see, the crystal structure PDB 5BT3.

FIG. 3QQQQ provides non-limiting examples of PCAF Targeting Ligands wherein R represents exemplary points at which the Linker can be attached. See for example, M. Ghizzoni et al. Bioorg. Med Chem. 18: 5826-5834 (2010).

FIG. 3RRRR provides non-limiting examples of PHIP Targeting Ligands wherein R represents exemplary points at which the Linker can be attached. For additional examples and related ligands, see, Mol Cancer Ther. 7(9): 2621-2632 (2008).

FIG. 3SSSS provides non-limiting examples of TAF1 and TAF1L Targeting Ligands wherein R represents exemplary points at which the Linker can be attached. For additional examples and related ligands, see, Picaud S. et al. Sci Adv 2: e1600760-e1600760 (2016).

FIG. 3TTTT provides non-limiting examples of Histone Deacetylase 2 (HDAC2) Targeting Ligands wherein R represents exemplary points at which the Linker can be attached.

For additional examples and related ligands, see, Lauffer B. E. J. Biol. Chem. 288: 26926-26943 (2013); Wagner F. F. Bioorg. Med Chem. 24: 4008-4015 (2016); Bressi J. C. Bioorg. Med Chem. Lett. 20: 3142-3145 (2010); and, Lauffer B. E. J. Biol. Chem. 288: 26926-26943 (2013).

FIG. 3UUUU-3VVVV provide non-limiting examples of Histone Deacetylase 4 (HDAC4) Targeting Ligands wherein R represents exemplary points at which the Linker can be attached. For additional examples and related ligands, see, Burli R. W. J. Med Chem. 56: 9934 (2013); Luckhurst C. A. ACS Med Chem. Lett. 7: 34 (2016); Bottomley M. J. J. Biol. Chem. 283: 26694-26704 (2008).

FIG. 3WWWW provides non-limiting examples of Histone Deacetylase 6 Targeting Ligands wherein R represents exemplary points at which the Linker can be attached. For additional examples and related ligands, see, Harding R. J. (to be published); Hai Y. Nat. Chem. Biol. 12: 741-747, (2016); and, Miyake Y. Nat. Chem. Biol. 12: 748 (2016).

FIG. 3XXXX-3YYYY provide non-limiting examples of Histone Deacetylase 7 Targeting Ligands wherein R represents exemplary points at which the Linker can be attached. For additional examples and related ligands, see, Lobera M. Nat. Chem. Biol. 9: 319 (2013) and Schuetz A. J. Biol. Chem. 283: 11355-11363 (2008).

FIG. 3ZZZZ-3DDDDD provide non-limiting examples of Histone Deacetylase 8 Targeting Ligands wherein R represents exemplary points at which the Linker can be attached. For additional examples and related ligands, see, Whitehead L. Biol. Med. Chem. 19: 4626-4634 (2011); Tabackman A. A. J. Struct. Biol. 195: 373-378 (2016); Dowling D. P. Biochemistry 47, 13554-13563 (2008); Somoza J. R. Biochemistry 12, 1325-1334 (2004); Decroos C. Biochemistry 54: 2126-2135 (2015); Vannini A. Proc. NatlAcad. Sci. 101: 15064 (2004); Vannini A. EMBO Rep. 8: 879 (2007); the crystal structure PDB 5BWZ; Decroos A. ACS Chem. Biol. 9: 2157-2164 (2014); Somoza J. R. Biochemistry 12: 1325-1334 (2004); Decroos C. Biochemistry 54: 6501-6513 (2015); Decroos A. ACS Chem. Biol. 9: 2157-2164 (2014); and, Dowling D. P. Biochemistry 47: 13554-13563 (2008).

FIG. 3EEEEE provides non-limiting examples of Histone Acetyltransferase (KAT2B) Targeting Ligands wherein R represents exemplary points at which the Linker can be attached. For additional examples and related ligands, see, Chaikuad A. J. Med. Chem. 59: 1648-1653 (2016); the crystal structure PDB 1ZS5; and, Zeng L. J. Am. Chem. Soc. 127: 2376-2377 (2005).

FIG. 3FFFFF-3GGGGG provide non-limiting examples of Histone Acetyltransferase (KAT2A) Targeting Ligands wherein R represents exemplary points at which the Linker can be attached. For additional examples and related ligands, see, Ringel A. E. Acta Crystallogr. D. Struct. Biol. 72: 841-848 (2016).

FIG. 3HHHHH provides non-limiting examples of Histone Acetyltransferase Type B Catalytic Unit (HAT1) Targeting Ligands wherein R represents exemplary points at which the Linker can be attached. For additional examples and related ligands, see, the crystal structure PDB 2POW.

FIG. 31111I provides non-limiting examples of Cyclic AMP-dependent Transcription Factor (ATF2) Targeting Ligands wherein R represents exemplary points at which the Linker can be attached.

FIG. 3JJJJJ provides non-limiting examples of Histone Acetyltransferase (KAT5) Targeting Ligands wherein R represents exemplary points at which the Linker can be attached.

FIG. 3KKKKK-3MMMMM provide non-limiting examples of Lysine-specific histone demethylase 1A (KDM1A) Targeting Ligands wherein R represents exemplary points at which the Linker can be attached. For additional examples and related ligands, see, Mimasu S. Biochemistry 49: 6494-6503 (2010); Sartori L. J. Med. Chem. 60:1673-1693 (2017); and, Vianello P. J. Med. Chem. 60: 1693-1715 (2017).

FIG. 3NNNNN provides non-limiting examples of HDAC6 Zn Finger Domain Targeting Ligands wherein R represents exemplary points at which the Linker can be attached.

FIG. 3OOOOO-3PPPPP provide non-limiting examples of general Lysine Methyltransferase Targeting Ligands wherein R represents exemplary points at which the Linker can be attached.

FIG. 3QQQQQ-3TTTTT provide non-limiting examples of DOT1L Targeting Ligands wherein R represents exemplary points at which the Linker can be attached, A is N or CH, and m is 0 1 2 3 4 5 6 7 or 8. For additional examples and related ligands, see, the crystal structure PDB 5MVS (“Dot1L in complex with adenosine and inhibitor CPD1” Mobitz, H. et al. ACS Med Chem Lett., 2017, 8: 338-343); the crystal structure PDB 5MW3, 5MW4 (“Dot1L in complex inhibitor CPD7” Be C. et al.); the crystal structure PDB 5DRT (“Dot1L in complex inhibitor CPD2” Chen, C., et al. ACS Med Chem Lett., 2016, 7: 735-740); the crystal structure PDB 5DRY (“Dot1L in complex with CPD3”, Chen, C., et al. ACS Med Chem Lett., 2016, 7: 735-740), the crystal structure of PDB 5DSX (“Dot1L in complex with CPD10”, Chen, C., et al. ACS Med Chem Lett., 2016, 7: 735-740), the crystal structure PDB 5DT2 (“Dot1L in complex with CPD11”, Chen, C., et al. ACS Med Chem Lett., 2016, 7: 735-740), the crystal structure PDB 5JUW “(Dot1L in complex with SS148” Yu W. et al. Structural Genomics Consortium), the crystal structure PDB 6TE6 (“Dot1L in complex with an inhibitor, compound 3”, Stauffer, F., et al. ACS Med Chem Lett., 2019, 10: 1655-1660).

FIG. 3UUUUU provides non-limiting examples of EHMT1 Targeting Ligands wherein R represents exemplary points at which the Linker can be attached. For additional examples and related ligands, see, the crystal structure PDB 5TUZ (“EHMT1 in complex with inhibitor MS0124”, Babault N. et al.).

FIG. 3VVVVV provides non-limiting examples of EHMT2 Targeting Ligands wherein R represents exemplary points at which the Linker can be attached. For additional examples and related ligands, see, the crystal structure PDB 5TUY (“EHMT2 in complex with inhibitor MS0124”, Babault N. et al.); the PDB crystal structure 5TTF (“EHMT2 in complex with inhibitor MS012”, Dong A. et al.); the PDB crystal structure 3RJW (Dong A. et al. Structural Genomics Consortium); the PDB crystal structure 3K5K; Liu F. et al. J. Med. Chem. 52: 7950-7953 (2009); and, the PDB crystal structure 4NVQ (“EHMT2 in complex with inhibitor A-366” Sweis R.F. et al.).

FIG. 3WWWWW provides non-limiting examples of SETD2 Targeting Ligands wherein R represents exemplary points at which the Linker can be attached. For additional examples and related ligands, see, the PDB crystal structure 5LSY (“SETD2 in complex with cyproheptadine”, Tisi D. et al.); Tisi D. et al. ACS Chem. Biol. 11: 3093-3105 (2016); the crystal structures PDB 5LSS, 5LSX, 5LSZ, 5LT6, 5LT7, and 5LT8; the PDB crystal structure 4FMU; and, Zheng W. et al. J. Am. Chem. Soc. 134: 18004-18014 (2012).

FIG. 3XXXXX-3YYYYY provide non-limiting examples of SETD7 Targeting Ligands wherein R represents exemplary points at which the Linker can be attached. For additional examples and related ligands, see, the PDB crystal structure 5AYF (“SETD7 in complex with cyproheptadine.” Niwa H. et al.); the PDB crystal structure 4JLG (“SETD7 in complex with (R)-PFI-2”, Dong A. et al.); the PDB crystal structure 4JDS (Dong A. et. al Structural Genomics Consortium); the PDB crystal structure 4E47 (Walker J.R. et al. Structural Genomics Consortium; the PDB crystal structure 3VUZ (“SETD7 in complex with AAM-1.” Niwa H. et al.); the PDB crystal structure 3VVO; and, Niwa H et al. Acta Crystallogr. Sect.D 69: 595-602 (2013).

FIG. 3ZZZZZ provides non-limiting examples of SETD8 Targeting Ligands wherein R represents exemplary points at which the Linker can be attached. For additional examples and related ligands, see, the PDB crystal structure 5TH7 (“SETD8 in complex with MS453”, Yu W. et al.) and the PDB crystal structure 5T5G (Yu W et. al.; to be published).

FIG. 4A-4B provides non-limiting examples of SETDB1 Targeting Ligands wherein R represents exemplary points at which the Linker can be attached. For additional examples and related ligands, see, the PDB crystal structure 5KE2 (“SETDB1 in complex with inhibitor XST06472A”, Iqbal A. et al.); the PDB crystal structure 5KE3 (“SETDB1 in complex with fragment MRT0181a”, Iqbal A. et al.); the PDB crystal structure 5KH6 (“SETDB1 in complex with fragment methyl 3-(methylsulfonylamino)benzoate”, Walker J.R. et al. Structural Genomics Consortium); and, the PDB crystal structure 5KCO (“SETDB1 in complex with [N]-(4-chlorophenyl)methanesulfonamide”, Walker J.R. et al.) FIG. 4C-4P provides non-limiting examples of SMYD2 Targeting Ligands wherein R represents exemplary points at which the Linker can be attached. For additional examples and related ligands, see, the PDB crystal structure 5KJK (“SMYD2 in complex with inhibitor AZ13450370”, Cowen S.D. et al.); the PDB crystal structure 5KJM (“SMYD2 in complex with AZ931”, Cowen S.D. et al.); the PDB crystal structure 5KJN (“SMYD2 in complex with AZ506”, Cowen S.D. et al.); the PDB crystal structure 5ARF (“SMYD2 in complex with N-[3-(4-chlorophenyl)-1-{N′-cyano-N-[3-(difluoromethoxy)phenyl]carbamimidoyl}-4 5-dihydro-1H-pyrazol-4-YL]-N-ethyl-2-hydroxyacetamide”, Eggert E. et al.); the PDB crystal structure 5ARG (“SMYD2 in complex with BAY598”, Eggert E. et al.); the PDB crystal structure 4YND (“SMYD2 in complex with A-893”, Sweis R.F. et al.); the PDB crystal structure 4WUY (“SMYD2 in complex with LLY-507”, Nguyen H. et al.); and, the PDB crystal structure 3S7B (“N-cyclohexyl-N~3~-[2-(3 4-dichlorophenyl)ethyl]- N-(2-{[2-(5-hydroxy-3-oxo-3 4-dihydro-2H- 1 4-benzoxazin-8-yl)ethyl]amino}ethyl)-beta- alaninamide”, Ferguson A.D. et al.).

FIG. 4Q-4R provide non-limiting examples of SMYD3 Targeting Ligands wherein R represents exemplary points at which the Linker can be attached. For additional examples and related ligands, see, the crystal structure 5H17 (“SMYD3 in complex with 5′-{[(3S)-3-amino-3-carboxypropyl][3-(dimethylamino)propyl]amino}- 5′-deoxyadenosine”, Van Aller G.S. et al.); the crystal structure 5CCL (“SMYD3 in complex with oxindole compound”, Mitchell L.H. et al.); and, the crystal structure 5CCM (“Crystal structure of SMYD3 with SAM and EPZ030456”).

FIG. 4S provides non-limiting examples of SUV4-20H1 Targeting Ligands wherein R represents exemplary points at which the Linker can be attached. For additional examples and related ligands, see, the PDB crystal structure 5CPR (“SUV4-20H1 in complex with inhibitor A-196”, Bromberg K.D. et al.).

FIG. 4T-4AA provide non-limiting examples of Wild Type Androgen Receptor Targeting Ligands wherein R represents exemplary points at which the Linker can be attached. For additional examples and related ligands, see, the PDB crystal structures 5T8E and 5T8J (“Androgen Receptor in complex with 4-(pyrrolidin-1-yl)benzonitrile derivatives”, Asano M. et al.); Asano M. et al. Bioorg. Med. Chem. Lett. 27: 1897-1901 (2017); the PDB crystal structure 5JJM (“Androgen Receptor”, Nadal M. et al.); the PDB crystal structure 5CJ6 (“Androgen Receptor in complex with 2-Chloro-4-[[(1R 2R)-2-hydroxy-2-methyl-cyclopentyl]amino]-3-methyl-benzonitrile derivatives”, Saeed A. et al.); the PDB crystal structure 4QL8 (“Androgen Receptor in complex with 3-alkoxy-pyrrolo[1 2-b]pyrazolines derivatives”, Ullrich T. et al.); the PDB crystal structure 4HLW (“Androgen Receptor Binding Function 3 (BF3) Site of the Human Androgen Receptor through Virtual Screening”, Munuganti R.S. et al.); the PDB crystal structure 3V49 (“Androgen Receptor lbd with activator peptide and sarm inhibitor 1”, Nique F. et al.); Nique F. et al. J. Med. Chem. 55: 8225-8235 (2012); the PDB crystal structure 2YHD (“Androgen Receptor in complex with AF2 small molecule inhibitor”, Axerio-Cilies P. et al.); the PDB crystal structure 3RLJ (“Androgen Receptor ligand binding domain in complex with SARM S-22”, Bohl C.E. et al.); Bohl C.E. et al. J. Med. Chem. 54: 3973-3976 (2011); the PDB crystal structure 3B5R (“Androgen Receptor ligand binding domain in complex with SARM C-31”, Bohl C.E. et al.); Bohl C.E. et al. Bioorg. Med. Chem. Lett. 18: 5567-5570 (2008); the PDB crystal structure 2PIP (“Androgen Receptor ligand binding domain in complex with small molecule”, Estebanez-Perpina E. et al.); Estebanez-Perpina. E. Proc. Natl. Acad. Sci. 104:16074-16079 (2007); the PDB crystal structure 2PNU (“Androgen Receptor ligand binding domain in complex with EM5744”, Cantin L. et al.); and, the PDB crystal structure 2HVC (“Androgen Receptor ligand binding domain in complex with LGD2226”, Wang F. et al.). For additional related ligands, see, Matias P.M. et al. “Structural Basis for the Glucocorticoid Response in a Mutant Human Androgen Receptor (Ar(Ccr)) Derived from an Androgen-Independent Prostate Cancer.” J Med. Chem. 45: 1439 (2002); Sack J.S. et al. “Crystallographic structures of the ligand-binding domains of the androgen receptor and its T877A mutant complexed with the natural agonist dihydrotestosterone.” Proc. Natl. Acad. Sci. 98: 4904-4909 (2001); He B. et al. “Structural basis for androgen receptor interdomain and coactivator interactions suggests a transition in nuclear receptor activation function dominance.” Mol. Cell 16: 425-438 (2004); Pereira de Jesus-Tran K. “Comparison of crystal structures of human androgen receptor ligand-binding domain complexed with various agonists reveals molecular determinants responsible for binding affinity.” Protein Sci. 15: 987-999 (2006); Bohl C.E. et al. “Structural Basis for Accommodation of Nonsteroidal Ligands in the Androgen Receptor.” Mol Pharmacol. 63(1):211-23 (2003); Sun C. et al. “Discovery of potent orally-active and muscle-selective androgen receptor modulators based on an N-aryl-hydroxybicyclohydantoin scaffold.” J. Med. Chem. 49: 7596-7599 (2006); Nirschl A.A. et al. “N-aryl-oxazolidin-2-imine muscle selective androgen receptor modulators enhance potency through pharmacophore reorientation.” J Med. Chem. 52: 2794-2798 (2009); Bohl C.E. et al. “Effect of B-ring substitution pattern on binding mode of propionamide selective androgen receptor modulators.” Bioorg. Med. Chem. Lett. 18: 5567-5570 (2008); Ullrich T. et al. “3-alkoxy-pyrrolo[1 2-b]pyrazolines as selective androgen receptor modulators with ideal physicochemical properties for transdermal administration.” J. Med. Chem. 57: 7396-7411 (2014); Saeed A. et al. “2-Chloro-4-[[(1R 2R)-2-hydroxy-2-methyl-cyclopentyl]amino]-3-methyl-benzonitrile: A Transdermal Selective Androgen Receptor Modulator (SARM) for Muscle Atrophy.” J. Med. Chem. 59: 750-755 (2016); Nique et al. “Discovery of diarylhydantoins as new selective androgen receptor modulators.” J. Med. Chem. 55: 8225-8235 (2012); and, Michael E. Jung et al. “Structure-Activity Relationship for Thiohydantoin Androgen Receptor Antagonists for Castration-Resistant Prostate Cancer (CRPC).” J Med Chem. 53: 2779-2796 (2010).

FIG. 4BB provides non-limiting examples of Mutant T877A Androgen Receptor Targeting Ligands wherein R represents exemplary points at which the Linker can be attached. For additional examples and related ligands, see, the PDB crystal structure 40GH (Androgen Receptor T877A-AR-LBD”, Hsu C.L. et al.) and the PDB crystal structure 20Z7 (“Androgen Receptor T877A-AR-LBD”, Bohl C.E. et al.).

FIG. 4CC provides non-limiting examples of Mutant W741L Androgen Receptor Targeting Ligands wherein R represents exemplary points at which the Linker can be attached. For additional examples and related ligands, see, the PDB crystal structure 40JB (“Androgen Receptor T877A-AR-LBD”, Hsu C.L. et al.).

FIG. 4DD-4EE provide non-limiting examples of Estrogen and/or Androgen Targeting Ligands wherein R represents exemplary points at which the Linker can be attached.

FIG. 5A provides non-limiting examples of Afatinib, a Targeting Ligand for the EGFR and ErbB2/4 receptors. R represents exemplary points at which the Linker can be attached.

FIG. 5B provides non-limiting examples of Axitinib, a Targeting Ligand for the VEGFR1/2/3, PDGFRβ, and Kit receptors. R represents exemplary points at which the Linker can be attached.

FIG. 5C-5D provide non-limiting examples of Bosutinib, a Targeting Ligand for the BCR-Abl, Src, Lyn and Hck receptors. R represents exemplary points at which the Linker can be attached.

FIG. 5E provides non-limiting examples of Cabozantinib, a Targeting Ligand for the RET, c-Met, VEGFR1/2/3, Kit, TrkB, Flt3, Axl, and Tie 2 receptors. R represents exemplary points at which the Linker can be attached.

FIG. 5F provides non-limiting examples of Ceritinib, a Targeting Ligand for the ALK, IGF-1R, InsR, and ROSI receptors. R represents exemplary points at which the Linker can be attached.

FIG. 5G provides non-limiting examples of Crizotinib, a Targeting Ligand for the ALK, c-Met, HGFR, ROSI, and MST1R receptors. R represents exemplary points at which the Linker can be attached.

FIG. 5H provides non-limiting examples of Dabrafenib, a Targeting Ligand for the B-Raf receptor. R represents exemplary points at which the Linker can be attached.

FIG. 5I provides non-limiting examples of Dasatinib, a Targeting Ligand for the BCR-Abl, Src, Lck, Lyn, Yes, Fyn, Kit, EphA2, and PDGFRP receptors. R represents exemplary points at which the Linker can be attached.

FIG. 5J provides non-limiting examples of Erlotinib, a Targeting Ligand for the EGFR receptor. R represents exemplary points at which the Linker can be attached. FIG. 5K-5M provide non-limiting examples of Everolimus, a Targeting Ligand for the HER2 breast cancer receptor, the PNET receptor, the RCC receptors, the RAML receptor, and the SEGA receptor. R represents exemplary points at which the Linker can be attached.

FIG. 5N provides non-limiting examples of Gefitinib, a Targeting Ligand for the EGFR and PDGFR receptors. R represents exemplary points at which the Linker can be attached.

FIG. 5O provides non-limiting examples of Ibrutinib, a Targeting Ligand for the BTK receptor. R represents exemplary points at which the Linker can be attached.

FIG. 5P-5Q provide non-limiting examples of Imatinib, a Targeting Ligand for the BCR-Abl, Kit, and PDGFR receptors. R represents exemplary points at which the Linker can be attached.

FIG. 5R-5S provide non-limiting examples of Lapatinib, a Targeting Ligand for the EGFR and ErbB2 receptors. R represents exemplary points at which the Linker can be attached.

FIG. 5T provides non-limiting examples of Lenvatinib, a Targeting Ligand for the VEGFR1/2/3, FGFR1/2/3/4, PDGFRα, Kit, and RET receptors. R represents exemplary points at which the Linker can be attached.

FIG. 5U-5V provide non-limiting examples of Nilotinib, a Targeting Ligand for the BCR-Abl, PDGRF, and DDR1 receptors. R represents exemplary points at which the Linker can be attached.

FIG. 5W-5X provide non-limiting examples of Nintedanib, a Targeting Ligand for the FGFR1/2/3, Flt3, Lck, PDGFRα/β, and VEGFR1/2/3 receptors. R represents exemplary points at which the Linker can be attached.

FIG. 5Y-5Z provide non-limiting examples of Palbociclib, a Targeting Ligand for the CDK4/6 receptor. R represents exemplary points at which the Linker can be attached.

FIG. 5AA provides non-limiting examples of Pazopanib, a Targeting Ligand for the VEGFR1/2/3, PDGFRα/β, FGFR1/3, Kit, Lck, Fms, and Itk receptors. R represents exemplary points at which the Linker can be attached.

FIG. 5BB-5CC provide non-limiting examples of Ponatinib, a Targeting Ligand for the BCR-Abl, T315I VEGFR, PDGFR, FGFR, EphR, Src family kinases, Kit, RET, Tie2, and Flt3 receptors. R represents exemplary points at which the Linker can be attached.

FIG. 5DD provides non-limiting examples of Regorafenib, a Targeting Ligand for the VEGFR1/2/3, BCR-Abl, B-Raf, B-Raf (V600E), Kit, PDGFRα/β, RET, FGFR1/2, Tie2, and Eph2A. R represents exemplary points at which the Linker can be attached.

FIG. 5EE provides non-limiting examples of Ruxolitinib, a Targeting Ligand for the JAK1/2 receptors. R represents exemplary points at which the Linker can be attached.

FIG. 5FF-5GG provide non-limiting examples of Sirolimus, a Targeting Ligand for the FKBP12/mTOR receptors. R represents exemplary points at which the Linker can be attached.

FIG. 5HH provides non-limiting examples of Sorafenib, a Targeting Ligand for the B-Raf, CDK8, Kit, Flt3, RET, VEGFR1/2/3, and PDGFR receptors. R represents exemplary points at which the Linker can be attached.

FIG. 511-5JJ provide non-limiting examples of Sunitinib, a Targeting Ligand for PDGFRα/β, VEGFR1/2/3, Kit, Flt3, CSF-1R, RET. R represents exemplary points at which the Linker can be attached.

FIG. 5KK-5LL provide non-limiting examples of Temsirolimus, a Targeting Ligand FKBP12/mTOR. R represents exemplary points at which the Linker can be attached.

FIG. 5MM provides non-limiting examples of Tofacitinib, a Targeting Ligand for JAK3 receptors. R represents exemplary points at which the Linker can be attached.

FIG. 5NN provides non-limiting examples of Trametinib, a Targeting Ligand for the MEK1/2 receptors. R represents exemplary points at which the Linker can be attached.

FIG. 5OO-5PP provide non-limiting examples of Vandetanib, a Targeting Ligand for the EGFR, VEGFR, RET, Tie2, Brk, and EphR. R represents exemplary points at which the Linker can be attached.

FIG. 5QQ provides non-limiting examples of Vemurafenib, a Targeting Ligand for the A/B/C-Raf, KSR1, and B-Raf (V600E) receptors. R represents exemplary points at which the Linker can be attached.

FIG. 5RR provides non-limiting examples of Idelasib, a Targeting Ligand for the PI3Ka receptor. R represents exemplary points at which the Linker can be attached.

FIG. 5SS provides non-limiting examples of Buparlisib, a Targeting Ligand for the PI3Ka receptor. R represents exemplary points at which the Linker can be attached.

FIG. 5TT provides non-limiting examples of Taselisib, a Targeting Ligand for the PI3Ka receptor. R represents exemplary points at which the Linker can be attached.

FIG. 5UU provides non-limiting examples of Copanlisib, a Targeting Ligand for the PI3Ka. R represents exemplary points at which the Linker can be attached.

FIG. 5VV provides non-limiting examples of Alpelisib, a Targeting Ligand for the PI3Ka. R represents exemplary points at which the Linker can be attached.

FIG. 5WW provides non-limiting examples of Niclosamide, a Targeting Ligand for the CNNTB1. R represents exemplary points at which the Linker can be attached.

FIG. 6A-6B provide nonlimiting examples of the BRD4 Bromodomains of PCAF and GCN5 receptors 1 Targeting Ligands wherein R represents exemplary points at which the Linker can be attached. For additional examples and related ligands, see, the PDB crystal structure 5tpx (“Discovery of a PCAF Bromodomain Chemical Probe”); Moustakim, M., et al. Angew. Chem. Int. Ed. Engl. 56: 827 (2017); the PDB crystal structure 5mlj (“Discovery of a Potent, Cell Penetrant, and Selective p300/CBP-Associated Factor (PCAF)/General Control Nonderepressible 5 (GCN5) Bromodomain Chemical Probe”); and, Humphreys, P. G. et al. J. Med. Chem. 60: 695 (2017).

FIG. 6C-6D provide nonlimiting examples of G9a (EHMT2) Targeting Ligands wherein R represents exemplary points at which the Linker can be attached. For additional examples and related ligands, see, the PDB crystal structure 3k5k; (“Discovery of a 2,4-diamino-7-aminoalkoxyquinazoline as a potent and selective inhibitor of histone lysine methyltransferase G9a”); Liu, F. et al. J. Med. Chem. 52: 7950 (2009); the PDB crystal structure 3rjw (“A chemical probe selectively inhibits G9a and GLP methyltransferase activity in cells”); Vedadi, M. et al. Nat. Chem. Biol. 7: 566 (2011); the PDB crystal structure 4nvq (“Discovery and development of potent and selective inhibitors of histone methyltransferase g9a”); and, Sweis, R. F. et al. ACS Med Chem Lett 5: 205 (2014).

FIG. 6E-6G provide nonlimiting examples of EZH2 Targeting Ligands wherein R represents exemplary points at which the Linker can be attached. For additional examples and related ligands, see, the PDB crystal structure 5ij8 (“Polycomb repressive complex 2 structure with inhibitor reveals a mechanism of activation and drug resistance”); Brooun, A. et al. Nat Commun 7: 11384 (2016); the PDB crystal structure 51s6 (“Identification of (R)-N-((4-Methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide (CPI-1205), a Potent and Selective Inhibitor of Histone Methyltransferase EZH2, Suitable for Phase I Clinical Trials for B-Cell Lymphomas”); Vaswani, R. G. et al. J. Med. Chem. 59: 9928 (2016); and, the PDB crystal structures 5ij8 and 51s6.

FIG. 6H-6I provide non-limiting examples of EED Targeting Ligands wherein R represents exemplary points at which the Linker can be attached. For additional examples and related ligands, see, the PDB crystal structures 5h15 and 5h19 (“Discovery and Molecular Basis of a Diverse Set of Polycomb Repressive Complex 2 Inhibitors Recognition by EED”); Li, L. et al. PLoS ONE 12: e0169855 (2017); and, the PDB crystal structure 5hl9.

FIG. 6J provides non-limiting examples of KMT5A (SETD8) Targeting Ligands wherein R represents exemplary points at which the Linker can be attached. See for example, the PDB crystal structure 5t5g.

FIG. 6K-6L provide non-limiting examples of DOT1L Targeting Ligands wherein R represents exemplary points at which the Linker can be attached. For additional examples and related ligands, see, the PDB crystal structure 4eki (“Conformational adaptation drives potent, selective and durable inhibition of the human protein methyltransferase DOTIL”); Basavapathruni, A. et al. Chem. Biol. DrugDes. 80: 971 (2012); the PDB crystal structure 4hra (“Potent inhibition of DOT 1L as treatment of MLL-fusion leukemia”); Daigle, S. R. et al. Blood 122: 1017 (2013); the PDB crystal structure 5dry (“Discovery of Novel Dot1L Inhibitors through a Structure-Based Fragmentation Approach”) Chen, C. et al. ACSMed. Chem. Lett. 7: 735 (2016); the PDB crystal structure 5dt2 (“Discovery of Novel Dot1L Inhibitors through a Structure-Based Fragmentation Approach”); and, Chen, C. et al. ACSMed. Chem. Lett. 7: 735 (2016).

FIG. 6M-6N provide nonlimiting examples of PRMT3 Targeting Ligands wherein R represents exemplary points at which the Linker can be attached. For additional examples and related ligands, see, the PDB crystal structure 3smq (“An allosteric inhibitor of protein arginine methyltransferase 3”); Siarheyeva, A. et al. Structure 20: 1425 (2012); PDB crystal structure 4ryl (“A Potent, Selective and Cell-Active Allosteric Inhibitor of Protein Arginine Methyltransferase 3 (PRMT3)”); and Kaniskan, H. U. et al. Angew. Chem. Int. Ed. Engl. 54: 5166 (2015).

FIG. 6O provides non-limiting examples of CARM1 (PRMT4) Targeting Ligands wherein R represents exemplary points at which the Linker can be attached. For additional examples and related ligands, see, the PDB crystal structures 2ylx and 2ylw and related ligands described in “Structural Basis for Carm1 Inhibition by Indole and Pyrazole Inhibitors.” Sack, J. S. et al. Biochem. J. 436: 331 (2011).

FIG. 6P provides non-limiting examples of PRMT5 Targeting Ligands wherein R represents exemplary points at which the Linker can be attached. For additional examples and related ligands, see, the PDB crystal structure 4x61 and related ligands described in “A selective inhibitor of PRMT5 with in vivo and in vitro potency in MCL models”. Chan-Penebre, E. Nat. Chem. Biol. 11: 432 (2015).

FIG. 6Q provides non-limiting examples of PRMT6 Targeting Ligands wherein R represents exemplary points at which the Linker can be attached. For additional examples and related ligands, see, the PDB crystal structure 4y30 and related ligands described in “Aryl Pyrazoles as Potent Inhibitors of Arginine Methyltransferases: Identification of the First PRMT6 Tool Compound”. Mitchell, L. H. et al. ACSMed. Chem. Lett. 6: 655 (2015).

FIG. 6R provides non-limiting examples of LSD1 (KDM1A) Targeting Ligands wherein R represents exemplary points at which the Linker can be attached. For additional examples and related ligands, see, the PDB crystal structure 51gu and related ligands described in “Thieno[3,2-b]pyrrole-5-carboxamides as New Reversible Inhibitors of Histone Lysine Demethylase KDM1A/LSD1. Part 2: Structure-Based Drug Design and Structure-Activity Relationship”. Vianello, P. et al. J. Med. Chem. 60: 1693 (2017).

FIG. 6S-6T provides non-limiting examples of KDM4 Targeting Ligands wherein R 10 represents exemplary points at which the Linker can be attached. For additional examples and related ligands, see, the PDB crystal structure 3rvh; the PDB crystal structure 5a7p and related ligands described in “Docking and Linking of Fragments to Discover Jumonji Histone Demethylase Inhibitors.” Korczynska, M., et al. J. Med. Chem. 59: 1580 (2016); and, the PDB crystal structure 3f3c and related ligands described in “8-Substituted Pyrido[3,4-d]pyrimidin-4(3H)-one Derivatives As Potent, Cell Permeable, KDM4 (JMJD2) and KDM5 (JARID1) Histone Lysine Demethylase Inhibitors.” Bavetsias, V. et al. J. Med. Chem. 59: 1388 (2016).

FIG. 6U provides non-limiting examples of KDM5 Targeting Ligands wherein R represents exemplary points at which the Linker can be attached. For additional examples and related ligands, see, the PDB crystal structure 3fun and related ligands described in “Structural Analysis of Human Kdm5B Guides Histone Demethylase Inhibitor Development”. Johansson, C. et al. Nat. Chem. Biol. 12: 539 (2016) and the PDB crystal structure 5ceh and related ligands described in “An inhibitor of KDM5 demethylases reduces survival of drug-tolerant cancer cells”. Vinogradova, M. et al. Nat. Chem. Biol. 12: 531 (2016).

FIG. 6V-6W provide non-limiting examples of KDM6 Targeting Ligands wherein R represents exemplary points at which the Linker can be attached. For additional examples and related ligands, see, the PDB crystal structure 4ask and related ligands described in “A Selective Jumonji H3K27 Demethylase Inhibitor Modulates the Proinflammatory Macrophage Response”. Kruidenier, L. et al. Nature 488: 404 (2012).

FIG. 6X provides non-limiting examples of L3MBTL3 targeting ligands wherein R represents exemplary points at which the Linker can be attached. See for example, the PDB crystal structure 4fl6.

FIG. 6Y provides non-limiting examples of Menin Targeting Ligands wherein R represents exemplary points at which the Linker can be attached. For additional examples and related ligands, see, the PDB crystal structure 4x5y and related ligands described in “Pharmacologic Inhibition of the Menin-MLL Interaction Blocks Progression of MLL Leukemia In Vivo” Borkin, D. et al. Cancer Cell27: 589 (2015) and the PDB crystal structure 4og8 and related ligands described in “High-Affinity Small-Molecule Inhibitors of the Menin-Mixed Lineage Leukemia (MLL) Interaction Closely Mimic a Natural Protein-Protein Interaction” He, S. et al. J. Med. Chem. 57: 1543 (2014).

FIG. 6Z-6AA provide non-limiting examples of HDAC6 Targeting Ligands wherein R represents exemplary points at which the Linker can be attached. See for example, the PDB crystal structures 5kh3 and 5eei.

FIG. 6BB provides non-limiting examples of HDAC7 Targeting Ligands wherein R represents exemplary points at which the Linker can be attached. For additional examples and related ligands, see, the PDB crystal structure 3c10 and related ligands described in “Human HDAC7 harbors a class IIa histone deacetylase-specific zinc binding motif and cryptic deacetylase activity.” Schuetz, A. et al. J. Biol. Chem. 283: 11355 (2008) and the PDB crystal structure PDB 3zns and related ligands described in “Selective Class Iia Histone Deacetylase Inhibition Via a Non-Chelating Zinc Binding Group”. Lobera, M. et al. Nat. Chem. Biol. 9: 319 (2013).

FIG. 7A-7C provide non-limiting examples of Protein Tyrosine Phosphatase, Non-Receptor Type 1, PTP1B Targeting Ligands wherein R represents exemplary points at which the Linker can be attached. For additional examples and related ligands, see, the PDB crystal structure lbzj described in “Structural basis for inhibition of the protein tyrosine phosphatase 1B by phosphotyrosine peptide mimetics” Groves, M. R. et al. Biochemistry 37: 17773-17783 (1998); the PDB crystal structure 3cwe described in “Discovery of [(3-bromo-7-cyano-2-naphthyl)(difluoro)methyl]phosphonic acid, a potent and orally active small molecule PTP1B inhibitor”. Han Y, BioorgMed Chem Lett. 18:3200-5 (2008); the PDB crystal structures 2azr and 2b07 described in “Bicyclic and tricyclic thiophenes as protein tyrosine phosphatase 1B inhibitors.” Moretto, A. F. et al. Bioorg. Med. Chem. 14: 2162-2177 (2006); the PDB crystal structures PDB 2bgd, 2bge, 2cm7, 2cm8, 2cma, 2cmb, 2cmc described in “Structure-Based Design of Protein Tyrosine Phosphatase-1B Inhibitors”. Black, E. et al. Bioorg. Med. Chem. Lett. 15: 2503 (2005) and “Structural Basis for Inhibition of Protein-Tyrosine Phosphatase 1B by Isothiazolidinone Heterocyclic Phosphonate Mimetics.” Ala, P. J. et al. J Biol. Chem. 281: 32784 (2006); the PDB crystal structures 2f6t and 2f6w described in “1,2,3,4-Tetrahydroisoquinolinyl sulfamic acids as phosphatase PTP1B inhibitors”. Klopfenstein, S. R. et al. Bioorg. Med. Chem. Lett. 16: 1574-1578 (2006); the PDB crystal structures 2h4g, 2h4k, 2hbl described in “Monocyclic thiophenes as protein tyrosine phosphatase 1B inhibitors: Capturing interactions with Asp48.” Wan, Z. K. et al. Bioorg. Med. Chem. Lett. 16: 4941-4945 (2006); the PDB crystal structures 2zn7 described in “Structure-based optimization of protein tyrosine phosphatase-1 B inhibitors: capturing interactions with arginine 24”. Wan, Z. K. et al. Chem Med Chem. 3:1525-9 (2008); the PDB crystal structure 2nt7, 2nta described in “Probing acid replacements of thiophene PTP1B inhibitors.” Wan, Z. K. et al. Bioorg. Med. Chem. Lett. 17: 2913-2920 (2007); and, WO 2008148744 A1 assigned to Novartis AG titled “Thiadiazole derivatives as antidiabetic agents”. See also, the PDB crystal structures 1c84, 1c84, 1c85, 1c86, 1c88, 118g and described in ““2-(oxalylamino)-benzoic acid is a general, competitive inhibitor of protein-tyrosine phosphatases”. Andersen, H. S. et al. J. Biol. Chem. 275: 7101-7108 (2000); “Structure-based design of a low molecular weight, nonphosphorus, nonpeptide, and highly selective inhibitor of protein-tyrosine phosphatase 1B.” Iversen, L. F. et al. J. Biol. Chem. 275: 10300-10307 (2000); and, “Steric hindrance as a basis for structure-based design of selective inhibitors of protein-tyrosine phosphatases”. Iversen, L. F. et al. Biochemistry 40: 14812-14820 (2001).

FIG. 7D provides non-limiting examples of Tyrosine-protein phosphatase non-receptor type 11, SHP2 Targeting Ligands wherein R represents exemplary points at which the Linker can be attached. For additional examples and related ligands, see, the crystal structures PDB 4pvg and 305x and described in “Salicylic acid based small molecule inhibitor for the oncogenic Src homology-2 domain containing protein tyrosine phosphatase-2 (SHP2).” Zhang, X. et al. J. Med. Chem. 53: 2482-2493 (2010); and, the crystal structure PDB 5ehr and related ligands described in “Allosteric Inhibition of SHP2: Identification of a Potent, Selective, and Orally Efficacious Phosphatase Inhibitor.” Garcia Fortanet, J. et al. J. Med. Chem. 59: 7773-7782 (2016). Also, see the crystal structure PDB 5ehr described in “Allosteric Inhibition of SHP2: Identification of a Potent, Selective, and Orally Efficacious Phosphatase Inhibitor.” Garcia Fortanet, J. et al. J. Med. Chem. 59: 7773-7782 (2016) and “Allosteric inhibition of SHP2 phosphatase inhibits cancers driven by receptor tyrosine kinases.” Chen, Y. P. et al. Nature 535: 148-152 (2016).

FIG. 7E provides non-limiting examples of Tyrosine-protein phosphatase non-receptor type 22 Targeting Ligands wherein R represents exemplary points at which the Linker can be attached. For additional examples and related ligands, see, the crystal structure PDB 4j51 described in “A Potent and Selective Small-Molecule Inhibitor for the Lymphoid-Specific Tyrosine Phosphatase (LYP), a Target Associated with Autoimmune Diseases.” He, Y. et al. J. Med. Chem. 56: 4990-5008 (2013).

FIG. 7F provides non-limiting examples of Scavenger mRNA-decapping enzyme DcpS Targeting Ligands wherein R represents exemplary points at which the Linker can be attached. For additional examples and related ligands, see, the crystal structures PDB 3b17, 3b19, 3bla, 4qde, 4qdv, 4qeb and related ligands described in “DcpS as a therapeutic target for spinal muscular atrophy.” Singh, J. et al. ACS Chem.Biol. 3: 711-722 (2008).

FIG. 8A-8S provide non-limiting examples of BRD4 Bromodomain 1 Targeting Ligands wherein R represents exemplary points at which the Linker can be attached. For additional examples and related ligands, see, the crystal structures PDB 3u5k and 3u51 and related ligands in Filippakopoulos, P. et al. “Benzodiazepines and benzotriazepines as protein interaction inhibitors targeting bromodomains of the BET family”, Bioorg. Med. Chem. 20: 1878-1886 (2012); the crystal structure PDB 3u51; the crystal structure PDB 3zyu and related ligands described in Dawson, M.A. et al. “Inhibition of Bet Recruitment to Chromatin as an Effective Treatment for Mll-Fusion Leukaemia.” Nature 478: 529 (2011); the crystal structure PDB 4bwl and related ligands described in Mirguet, O._et al. “Naphthyridines as Novel Bet Family Bromodomain Inhibitors.” Chemmedchem 9: 589 (2014); the crystal structure PDB 4cfl and related ligands described in Dittmann, A. et al. “The Commonly Used Pi3-Kinase Probe Ly294002 is an Inhibitor of Bet Bromodomains” ACS Chem. Biol. 9: 495 (2014); the crystal structure PDB 4e96 and related ligands described in Fish, P. V. et al. “Identification of a chemical probe for bromo and extra C-terminal bromodomain inhibition through optimization of a fragment-derived hit.” J. Med. Chem. 55: 9831-9837 (2012); the crystal structure PDB 4clb and related ligands described in Atkinson, S. J. et al. “The Structure Based Design of Dual Hdac/Bet Inhibitors as Novel Epigenetic Probes.” Medchemcomm 5: 342 (2014); the crystal structure PDB 4f3i and related ligands described in Zhang, G. et al. “Down-regulation of NF-{kappa}B Transcriptional Activity in HIV-associated Kidney Disease by BRD4 Inhibition.” J. Biol. Chem. 287: 28840-28851 (2012); the crystal structure PDB 4hxl and related ligands described in Zhao, L._“Fragment-Based Drug Discovery of 2-Thiazolidinones as Inhibitors of the Histone Reader BRD4 Bromodomain.” J Med. Chem. 56: 3833-3851 (2013); the crystal structure PDB 4hxs and related ligands described in Zhao, L. et al. “Fragment-Based Drug Discovery of 2-Thiazolidinones as Inhibitors of the Histone Reader BRD4 Bromodomain.” J. Med. Chem. 56: 3833-3851 (2013); the crystal structure PDB 41rg and related ligands described in Gehling, V. S. et al. “Discovery, Design, and Optimization of Isoxazole Azepine BET Inhibitors.” ACS Med Chem Lett 4: 835-840 (2013); the crystal structure PDB 4mep and related ligands described in Vidler, L.R. “Discovery of Novel Small-Molecule Inhibitors of BRD4 Using Structure-Based Virtual Screening.” et al. J. Med. Chem. 56: 8073-8088 (2013); the crystal structures PDB 4nr8 and PDB 4c77 and related ligands described in Ember, S. W. et al. “Acetyl-lysine Binding Site of Bromodomain-Containing Protein 4 (BRD4) Interacts with Diverse Kinase Inhibitors”. ACS Chem.Biol. 9: 1160-1171 (2014); the crystal structure PDB 4o7a and related ligands described in Ember, S. W. et al. “Acetyl-lysine Binding Site of Bromodomain-Containing Protein 4 (BRD4) Interacts with Diverse Kinase Inhibitors.” ACS Chem. Biol. 9: 1160-1171 (2014); the crystal structure PDB 407b and related ligands described in “Acetyl-lysine Binding Site of Bromodomain-Containing Protein 4 (BRD4) Interacts with Diverse Kinase Inhibitors.” Ember, S. W. et al. (2014) ACS Chem. Biol. 9: 1160-1171; the crystal structure PDB 4o7c and related ligands described in Ember, S.W._et al. “Acetyl-lysine Binding Site of Bromodomain-Containing Protein 4 (BRD4) Interacts with Diverse Kinase Inhibitors”. ACS Chem. Biol. 9: 1160-1171 (2014); the crystal structure PDB 4gpj; the crystal structure PDB 4uix and related ligands described in Theodoulou, N.H._et al. “The Discovery of I-Brd9, a Selective Cell Active Chemical Probe for Bromodomain Containing Protein 9 Inhibition”. J. Med. Chem. 59: 1425 (2016); the crystal structure PDB 4uiz and related ligands described in Theodoulou, N.H., et al. “The Discovery of I-Brd9, a Selective Cell Active Chemical Probe for Bromodomain Containing Protein 9 Inhibition”. J. Med Chem. 59: 1425 (2016); the crystal structure PDB 4wiv and related ligands described in McKeown, M.R._et al. “Biased multicomponent reactions to develop novel bromodomain inhibitors.” J. Med Chem. 57: 9019-9027 (2014); the crystal structure PDB 4x2i and related ligands described in Taylor, A. M. et al. “Discovery of Benzotriazolo[4,3-d][1,4]diazepines as Orally Active Inhibitors of BET Bromodomains.” ACS Med Chem. Lett. 7: 145-150 (2016); the crystal structure PDB 4yh3; And related ligands described in Duffy, B.C._“Discovery of a new chemical series of BRD4(1) inhibitors using protein-ligand docking and structure-guided design.” Bioorg. Med Chem. Lett. 25: 2818-2823 (2015); the crystal structure PDB 4yh4 and related ligands described in Duffy, B.C. “Discovery of a new chemical series of BRD4(1) inhibitors using protein-ligand docking and structure-guided design.” Bioorg. Med Chem. Lett. 25: 2818-2823 (2015); the crystal structure PDB 4zlq and related ligands described in Taylor, A.M. “Discovery of Benzotriazolo[4,3-d][1,4]diazepines as Orally Active Inhibitors of BET Bromodomains.” ACS Med Chem. Lett. 7: 145-150 (2016); the crystal structure PDB 4zwl; the crystal structure PDB 5a5s and related ligands described in Demont, E.H. “Fragment-Based Discovery of Low-Micromolar Atad2 Bromodomain Inhibitors. J. Med Chem. 58: 5649 (2015); the crystal structure PDB 5a85 and related ligands described in Bamborough, P. “Structure-Based Optimization of Naphthyridones Into Potent Atad2 Bromodomain Inhibitors” J. Med Chem. 58: 6151 (2015); the crystal structure PDB 5acy and related ligands described in Sullivan, J.M. “Autism-Like Syndrome is Induced by Pharmacological Suppression of Bet Proteins in Young Mice.” J Exp. Med. 212: 1771 (2015); the crystal structure PDB 5ad2 and related ligands described in Waring, M. J. et al. “Potent and Selective Bivalent Inhibitors of Bet Bromodomains”. Nat. Chem. Biol. 12: 1097 (2016); the crystal structure PDB 5cfw and related ligands described in Chekler, E. L. et al. “Transcriptional Profiling of a Selective CREB Binding Protein Bromodomain Inhibitor Highlights Therapeutic Opportunities.” Chem. Biol. 22: 1588-1596 (2015); the crystal structure PDB 5cqt and related ligands described in Xue, X. et al. “Discovery of Benzo[cd]indol-2(1H)-ones as Potent and Specific BET Bromodomain Inhibitors: Structure-Based Virtual Screening, Optimization, and Biological Evaluation”. J Med. Chem. 59: 1565-1579 (2016); the crystal structure PDB 5d3r and related ligands described in Hugle, M. et al. “4-Acyl Pyrrole Derivatives Yield Novel Vectors for Designing Inhibitors of the Acetyl-Lysine Recognition Site of BRD4(1)”. J Med. Chem. 59: 1518-1530 (2016); the crystal structure PDB 5dlx and related ligands described in Milhas, S. et al. “Protein-Protein Interaction Inhibition (2P21)-Oriented Chemical Library Accelerates Hit Discovery.” (2016) ACS Chem.Biol. 11: 2140-2148; the crystal structure PDB 5dlz and related ligands described in Milhas, S. et al. “Protein-Protein Interaction Inhibition (2P21)-Oriented Chemical Library Accelerates Hit Discovery.” ACS Chem. Biol. 11: 2140-2148 (2016); the crystal structure PDB 5dw2 and related ligands described in Kharenko, O. A. et al. “RVX-297-a novel BD2 selective inhibitor of BET bromodomains.” Biochem. Biophys. Res. Commun. 477: 62-67 (2016); the crystal structure PDB 5dlx; the crystal structure PDB 5his and related ligands described in Albrecht, B. K. et al. “Identification of a Benzoisoxazoloazepine Inhibitor (CPI-0610) of the Bromodomain and Extra-Terminal (BET) Family as a Candidate for Human Clinical Trials.” J. Med. Chem. 59: 1330-1339 (2016); the crystal structure PDB 5ku3 and related ligands described in Crawford, T.D._et al. “Discovery of a Potent and Selective in Vivo Probe (GNE-272) for the Bromodomains of CBP/EP300”. J. Med. Chem. 59: 10549-10563 (2016); the crystal structure PDB 51j2 and related ligands described in Bamborough, P._et al. “A Chemical Probe for the ATAD2 Bromodomain.” Angew. Chem. Int. Ed. Engl. 55: 11382-11386 (2016); the crystal structure PDB 5dlx and related ligands described in Wang, L. “Fragment-based, structure-enabled discovery of novel pyridones and pyridone macrocycles as potent bromodomain and extra-terminal domain (BET) family bromodomain inhibitors”. J. Med. Chem. 10.1021/acs.jmedchem.7b00017 (2017); WO 2015169962 A1 titled “Benzimidazole derivatives as BRD4 inhibitors and their preparation and use for the treatment of cancer” assigned to Boehringer Ingelheim International GmbH, Germany; and, WO 2011143669 A2 titled “Azolodiazepine derivatives and their preparation, compositions and methods for treating neoplasia, inflammatory disease and other disorders” assigned to Dana-Farber Cancer Institute, Inc, USA.

FIG. 8T-8V provide non-limiting examples of ALK Targeting Ligands wherein R represents exemplary points at which the Linker can be attached. For additional examples and related ligands, see, the crystal structures PDB 2xb7 and 2xba and related ligands described in Bossi, R. T. et al. “Crystal Structures of Anaplastic Lymphoma Kinase in Complex with ATP Competitive Inhibitors” Biochemistry 49: 6813-6825 (2010); the crystal structures PDB 2yfx, 4ccb, 4ccu, and 4cd0 and related ligands described in Huang, Q. et al. “Design of Potent and Selective Inhibitors to Overcome Clinical Anaplastic Lymphoma Kinase Mutations Resistant to Crizotinib.” J. Med. Chem. 57: 1170 (2014); the crystal structures PDB, 4cli, 4cmo, and 4cnh and related ligands described in Johnson, T. W. et al. “Discovery of (10R)-7-Amino-12-Fluoro-2,10,16-Trimethyl-15-Oxo-10,15,16,17-Tetrahydro-2H-8,4-(Metheno)Pyrazolo[4,3-H][2,5,11]Benzoxadiazacyclotetradecine-3-Carbonitrile (Pf-06463922), a Macrocyclic Inhibitor of Alk/Ros1 with Pre-Clinical Brain Exposure and Broad Spectrum Potency Against Alk-Resistant Mutations.” J. Med Chem. 57: 4720 (2014); the crystal structure PDB 4fny and related ligands described in Epstein, L. F. et al. “The R1275Q Neuroblastoma Mutant and Certain ATP-competitive Inhibitors Stabilize Alternative Activation Loop Conformations of Anaplastic Lymphoma Kinase.” J. Biol. Chem. 287: 37447-37457 (2012). the crystal structure PDB 4dce and related ligands described in Bryan, M. C. et al “Rapid development of piperidine carboxamides as potent and selective anaplastic lymphoma kinase inhibitors. “J. Med Chem. 55: 1698-1705 (2012); the crystal structure PDB 4joa and related ligands described in Gummadi, V. R. et al. “Discovery of 7-azaindole based anaplastic lymphoma kinase (ALK) inhibitors: wild type and mutant (L1196M) active compounds with unique binding mode.” (2013) Bioorg. Med Chem. Lett. 23: 4911-4918; and, the crystal structure PDB 5iui and related ligands described in Tu, C.H. et al. “Pyrazolylamine Derivatives Reveal the Conformational Switching between Type I and Type II Binding Modes of Anaplastic Lymphoma Kinase (ALK).” J. Med Chem. 59: 3906-3919 (2016).

FIG. 8W-8X provide non-limiting examples of BTK Targeting Ligands wherein R represents exemplary points at which the Linker can be attached. For additional examples and related ligands, see, the crystal structure PDB 3gen, 3piz and related ligands described in Marcotte, D. J. et al. “Structures of human Bruton's tyrosine kinase in active and inactive conformations suggest a mechanism of activation for TEC family kinases.” Protein Sci. 19: 429-439 (2010) and Kuglstatter, A. et al. “Insights into the conformational flexibility of Bruton's tyrosine kinase from multiple ligand complex structures” Protein Sci. 20: 428-436” (2011); the crystal structure PDB 3ocs, 4ot6 and related ligands described in Lou, Y. et al. “Structure-Based Drug Design of RN486, a Potent and Selective Bruton's Tyrosine Kinase (BTK) Inhibitor, for the Treatment of Rheumatoid Arthritis” J. Med. Chem. 58: 512-516 (2015); the crystal structures PDB 5fbn and 5fbo and related ligands described in Liu, J. et al. “Discovery of 8-Amino-imidazo[1,5-a]pyrazines as Reversible BTK Inhibitors for the Treatment of Rheumatoid Arthritis.” ACS Med. Chem. Lett. 7: 198-203 (2016); the crystal structure PDB 3pix and related ligands described in Kuglstatter, A. et al. “Insights into the conformational flexibility of Bruton's tyrosine kinase from multiple ligand complex structures.” Protein Sci. 20: 428-436 (2011); and, the crystal structure PDB 3pij and related ligands described in Bujacz, A. et al. “Crystal structures of the apo form of beta-fructofuranosidase from Bifidobacterium longum and its complex with fructose. “Febs J. 278: 1728-1744 (2011).

FIG. 8Y provides non-limiting examples of FLT3 Targeting Ligands wherein R represents exemplary points at which the Linker can be attached. For additional examples and related ligands, see, the crystal structures PDB 4xuf and 4rt7 and related ligands described in Zorn, J. A. et al. “Crystal Structure of the FLT3 Kinase Domain Bound to the Inhibitor Quizartinib (AC220)”. Plos One 10: e0121177-e0121177 (2015).

FIG. 8Z-8AA provide non-limiting examples of TNIK Targeting Ligands wherein R represents exemplary points at which the Linker can be attached. For additional examples and related ligands, see, the crystal structure PDB 2x7f; the crystal structures PDB 5ax9 and 5d7a; and, related ligands described in Masuda, M. et al. “TNIK inhibition abrogates colorectal cancer stemness.” Nat Commun 7: 12586-12586 (2016).

FIG. 8BB-8CC provide non-limiting examples of NTRK1, NTRK2, and NTRK3 Targeting Ligands wherein R represents exemplary points at which the Linker can be attached. For additional examples and related ligands, see, the crystal structure PDB 4aoj and related ligands described in Wang, T. et al. “Discovery of Disubstituted Imidazo[4,5-B]Pyridines and Purines as Potent Trka Inhibitors.” ACSMed. Chem. Lett. 3: 705 (2012); the crystal structures PDB 4pmm, 4pmp, 4pms and 4pmt and related ligands described in Stachel, S. J. et al. “Maximizing diversity from a kinase screen: identification of novel and selective pan-Trk inhibitors for chronic pain.” J. Med. Chem. 57: 5800-5816 (2014); the crystal structures PDB 4yps and 4yne and related ligands described in Choi, H. S. et al. “(R)-2-Phenylpyrrolidine Substituted Imidazopyridazines: A New Class of Potent and Selective Pan-TRK Inhibitors.” ACSMed. Chem. Lett. 6: 562-567 (2015); the crystal structures PDB 4at5 and 4at3 and related ligands described in Bertrand, T. et al. “The Crystal Structures of Trka and Trkb Suggest Key Regions for Achieving Selective Inhibition.” J. Mol. Biol. 423: 439 (2012); and, the crystal structures PDB 3v5q and 4ymj and related ligands described in Albaugh, P. et al. “Discovery of GNF-5837, a selective TRK Inhibitor with efficacy in rodent cancer tumor models.” ACS Med. Chem. Lett. 3: 140-145 (2012) and Choi, H. S. et al. “(R)-2-Phenylpyrrolidine Substitute Imidazopyridazines: a New Class of Potent and Selective Pan-TRK Inhibitors.” ACS Med Chem Lett 6: 562-567 (2015).

FIG. 8DD-8EE provide non-limiting examples of FGFR1 Targeting Ligands wherein R represents exemplary points at which the Linker can be attached. For additional examples and related ligands, see, the crystal structures PDB 3tto and 2fgi and related ligands described in Brison, Y. et al. “Functional and structural characterization of alpha-(1-2) branching sucrase derived from DSR-E glucansucrase.” J. Biol. Chem. 287: 7915-7924 (2012) and Mohammadi, M. et al. “Crystal structure of an angiogenesis inhibitor bound to the FGF receptor tyrosine kinase domain.” EMBO J. 17: 5896-5904 (1998); the crystal structure PDB 4fb3; the crystal structure PDB 4rwk and related ligands described in Harrison, C. et al. “Polyomavirus large T antigen binds symmetrical repeats at the viral origin in an asymmetrical manner.” J. Virol. 87: 13751-13759 (2013); the crystal structure PDB 4rwl and related ligands described in Sohl, C. D. et al. “Illuminating the Molecular Mechanisms of Tyrosine Kinase Inhibitor Resistance for the FGFR1 Gatekeeper Mutation: The Achilles' Heel of Targeted Therapy.” ACS Chem. Biol. 10: 1319-1329 (2015); the crystal structure PDB 4uwc; the crystal structure PDB 4v01 and related ligands described in Tucker, J. A. et al. “Structural Insights Into Fgfr Kinase Isoform Selectivity: Diverse Binding Modes of Azd4547 and Ponatinib in Complex with Fgfrl and Fgfr4.” Structure 22: 1764 (2014).; the crystal structure PDB 5a46 and related ligands described in Klein, T. et al. “Structural and Dynamic Insights Into the Energetics of Activation Loop Rearrangement in Fgfrl Kinase.” Nat. Commun. 6: 7877 (2015); and, the crystal structure PDB 5ew8 and related ligands described in Patani, H. et al. “Landscape of activating cancer mutations in FGFR kinases and their differential responses to inhibitors in clinical use.” Oncotarget 7: 24252-24268 (2016).

FIG. 8FF provides non-limiting examples of FGFR2 and FGFR3 Targeting Ligands wherein R represents exemplary points at which the Linker can be attached. For additional examples and related ligands, see, the crystal structure PDB 2pvf and related ligands described in Chen, H. et al. “A molecular brake in the kinase hinge region regulates the activity of receptor tyrosine kinases.” Mol. Cell 27: 717-730 (2007); and “Structure-based drug design of 1,3,5-triazine and pyrimidine derivatives as novel FGFR3 inhibitors with high selectivity over VEGFR2” Bioorg Med Chem 2020, 28, 115453.

FIG. 8GG provides non-limiting examples of FGFR4 Targeting Ligands wherein R represents exemplary points at which the Linker can be attached. For additional examples and related ligands, see, the crystal structure PDB 4tyi and related ligands described in Lesca, E. et al. “Structural analysis of the human fibroblast growth factor receptor 4 kinase.” J. Mol. Biol. 426: 3744-3756 (2014).

FIG. 8HH-8II provide non-limiting examples of MET Targeting Ligands wherein R represents exemplary points at which the Linker can be attached. For additional examples and related ligands, see, the crystal structures PDB 3qti and 3zcl; the crystal structures PDB 4xmo, 4xyf, and 3zcl and related ligands described in Peterson, E. A. et al. “Discovery of Potent and Selective 8-Fluorotriazolopyridine c-Met Inhibitors.” J. Med. Chem. 58: 2417-2430 (2015) and Cui, J. J. et al. “Lessons from (S)-6-(1-(6-(1-Methyl-1H-Pyrazol-4-Yl)-[1,2, 4]Triazolo[4,3-B]Pyridazin-3-Yl)Ethyl)Quinoline (Pf-04254644), an Inhibitor of Receptor Tyrosine Kinase C-met with High Protein Kinase Selectivity But Broad Phosphodiesterase Family Inhibition Leading to Myocardial Degeneration in Rats.” J Med. Chem. 56: 6651 (2013); the crystal structure PDB 5eyd and related ligands described in Boezio, A. A. et al. “Discovery of (R)-6-(1-(8-Fluoro-6-(1-methyl-1H-pyrazol-4-yl)-[1,2,4]triazolo[4,3-a]pyridin-3-yl)ethyl)-3-(2-methoxyethoxy)-1,6-naphthyridin-5(6H)-one (AMG 337), a Potent and Selective Inhibitor of MET with High Unbound Target Coverage and Robust In Vivo Antitumor Activity.” J. Med. Chem. 59: 2328-2342 (2016); the crystal structure PDB 3ce3 and related ligands described in Kim, K.S. et al. “Discovery of pyrrolopyridine-pyridone based inhibitors of Met kinase: synthesis, X-ray crystallographic analysis, and biological activities.” J. Med. Chem. 51: 5330-5341 (2008); the crystal structure PDB 2rfn and related ligands described in Bellon, S. F. et al. “c-Met inhibitors with novel binding mode show activity against several hereditary papillary renal cell carcinoma-related mutations.” J. Biol. Chem. 283: 2675-2683 (2008); and, the crystal structure PDB 5dg5 and related ligands described in Smith, B. D. et al “Altiratinib Inhibits Tumor Growth, Invasion, Angiogenesis, and Microenvironment-Mediated Drug Resistance via Balanced Inhibition of MET, TIE2, and VEGFR2.”. Mol. Cancer Ther. 14: 2023-2034 (2015).

FIG. 8JJ provides non-limiting examples of JAK1 Targeting Ligands wherein R represents exemplary points at which the Linker can be attached. For additional examples and related ligands, see, the crystal structure PDB 4ivd and related ligands described in Zak, M. et al. “Identification of C-2 Hydroxyethyl Imidazopyrrolopyridines as Potent JAK1 Inhibitors with Favorable Physicochemical Properties and High Selectivity over JAK2.” J. Med. Chem. 56: 4764-4785 (2013); the crystal structure PDB 5ele and related ligands described in Vasbinder, M. M. et al. “Identification of azabenzimidazoles as potent JAK1 selective inhibitors.” Bioorg. Med. Chem. Lett. 26: 60-67 (2016); the crystal structure PDB 5hx8 and related ligands described in Simov, V., et al. “Structure-based design and development of (benz)imidazole pyridones as JAK1-selective kinase inhibitors.” Bioorg. Med. Chem. Lett. 26: 1803-1808 (2016); the crystal structure PDB 5hx8 and related ligands described in Caspers, N. L. et al. “Development of a high-throughput crystal structure-determination platform for JAK1 using a novel metal-chelator soaking system”. Acta Crystallogr. Sect. F 72: 840-845 (2016); and, Kettle, J. G. “Discovery of the JAK1 selective kinase inhibitor AZD4205”, AACR National Meeting, April 2017.

FIG. 8KK-8LL provide non-limiting examples of JAK2 Targeting Ligands wherein R represents exemplary points at which the Linker can be attached. For additional examples and related ligands, see, the crystal structure PDB 3ugc and related ligands described in Andraos, R. et al. “Modulation of activation-loop phosphorylation by JAK inhibitors is binding mode dependent.” Cancer Discov 2: 512-523 (2012); the crystal structures PDB 5cf4, 5cf5, 5cf6 and 5cf8 and related ligands described in Hart, A. C. et al. “Structure-Based Design of Selective Janus Kinase 2 Imidazo[4,5-d]pyrrolo[2,3-b]pyridine Inhibitors.” ACS Med. Chem. Lett. 6: 845-849 (2015); the crystal structure PDB 5aep and related ligands described in Brasca, M. G. et al “Novel Pyrrole Carboxamide Inhibitors of Jak2 as Potential Treatment of Myeloproliferative Disorders” Bioorg. Med Chem. 23: 2387 (2015); the crystal structures PDB 4ytf, 4yth and 4yti and related ligands described in Farmer, L. J. et al. “Discovery of VX-509 (Decernotinib): A Potent and Selective Janus Kinase 3 Inhibitor for the Treatment of Autoimmune Diseases.” J. Med Chem. 58: 7195-7216 (2015); the crystal structure PDB 4ytf, 4yth, 4yti and related ligands described in Menet, C. J. et al. “Triazolopyridines as Selective JAK1 Inhibitors: From Hit Identification to GLPG0634.” J. Med Chem. 57: 9323-9342 (2014); the crystal structure PDB 4ji9 and related ligands described in Siu, M. et al. “2-Amino-[1,2,4]triazolo[1,5-a]pyridines as JAK2 inhibitors.” Bioorg. Med Chem. Lett. 23: 5014-5021 (2013); and, the crystal structures PDB 3io7 and3iok and related ligands described in Schenkel, L. B. et al. “Discovery of potent and highly selective thienopyridine janus kinase 2 inhibitors.” J. Med. Chem. 54: 8440-8450 (2011).

FIG. 8MM provides non-limiting examples of JAK3 Targeting Ligands wherein R represents exemplary points at which the Linker can be attached. For additional examples and related ligands, see, the crystal structure PDB 3zc6 and related ligands described in Lynch, S. M. et al. “Strategic Use of Conformational Bias and Structure Based Design to Identify Potent Jak3 Inhibitors with Improved Selectivity Against the Jak Family and the Kinome.” Bioorg. Med Chem. Lett. 23: 2793 (2013); and, the crystal structures PDB 4hvd, 4i6q, and 3zep and related ligands described in Soth, M. et al. “3-Amido Pyrrolopyrazine JAK Kinase Inhibitors: Development of a JAK3 vs JAK1 Selective Inhibitor and Evaluation in Cellular and in Vivo Models.” J. Med. Chem. 56: 345-356 (2013) and Jaime-Figueroa, S. et al. “Discovery of a series of novel 5H-pyrrolo[2,3-b]pyrazine-2-phenyl ethers, as potent JAK3 kinase inhibitors.” Bioorg. Med. Chem. Lett. 23: 2522-2526 (2013).

FIG. 8NN-8OO provide non-limiting examples of KIT Targeting Ligands wherein R represents exemplary points at which the Linker can be attached. For additional examples and related ligands, see, the crystal structure PDB 1t46 and related ligands described in Mol, C. D. et al. “Structural basis for the autoinhibition and STI-571 inhibition of c-Kit tyrosine kinase.” J. Biol. Chem. 279: 31655-31663 (2004); and, the crystal structure PDB 4u0i and related ligands described in Garner, A. P. et al. “Ponatinib Inhibits Polyclonal Drug-Resistant KIT Oncoproteins and Shows Therapeutic Potential in Heavily Pretreated Gastrointestinal Stromal Tumor (GIST) Patients.” Clin. Cancer Res. 20: 5745-5755 (2014).

FIG. 88PP-8VV provide non-limiting examples of EGFR Targeting Ligands wherein R represents exemplary points at which the Linker can be attached. For additional examples and related ligands, see, the crystal structures PDB 5hcy, 4rj4, and 5cav; Heald, R., “Noncovalent Mutant Selective Epidermal Growth Factor Receptor Inhibitors: A Lead Optimization Case Study”, J. Med. Chem. 58, 8877-8895 (2015); Hanano, E. J., “Discovery of Selective and Noncovalent Diaminopyrimidine-Based Inhibitors of Epidermal Growth Factor Receptor Containing the T790M Resistance Mutation. “J. Med Chem., 57, 10176-10191 (2014); Chan, B. K. et al. “Discovery of a Noncovalent, Mutant-Selective Epidermal Growth Factor Receptor Inhibitor “J Med Chem. 59, 9080 (2016); the crystal structure PDB 5d41 and related ligands described in Jia, Y. et al. “Overcoming EGFR(T790M) and EGFR(C797S) resistance with mutant-selective allosteric inhibitors “Nature 534, 129 (2016); Ward, R. A. “Structure- and reactivity-based development of covalent inhibitors of the activating and gatekeeper mutant forms of the epidermal growth factor receptor (EGFR)” J. Med Chem. 56, 7025-7048 (2013); the crystal structure PDB 4zau and related ligands described in “Discovery of a Potent and Selective EGFR Inhibitor (AZD9291) of Both Sensitizing and T790M Resistance Mutations That Spares the Wild Type Form of the Receptor “J. Med Chem., 57 (20), 8249-8267 (2014); the crystal structure PDB 5em7 and related ligands described in Bryan, M. C. et al. “Pyridones as Highly Selective, Noncovalent Inhibitors of T790M Double Mutants of EGFR “ACS Med Chem. Lett., 7 (1), 100-104 (2016); the crystal structure PDB 3IKA and related ligands described in Zhou, W. et al. “Novel mutant-selective EGFR kinase inhibitors against EGFR T790M” Nature 462(7276), 1070-1074 (2009); the crystal structure see PDB 5feq and related ligands described in Lelais, G., J. “Discovery of (R,E)-N-(7-Chloro-1-(1-[4-(dimethylamino)but-2-enoyl]azepan-3-yl)-1H-benzo[d]imidazol-2-yl)-2-methylisonicotinamide (EGF816), a Novel, Potent, and WT Sparing Covalent Inhibitor of Oncogenic (L858R, exl9del) and Resistant (T790M) EGFR Mutants for the Treatment of 5 EGFR Mutant Non-Small-Cell Lung Cancers” Med. Chem., 59 (14), 6671-6689 (2016); Lee, H.-J. “Noncovalent Wild-type-Sparing Inhibitors of EGFR T790M” Cancer Discov. 3(2): 168-181 (2013); the crystal structure PDB 5j7 h and related ligands described in Huang, W-S. et al. “Discovery of Brigatinib (AP26113), a Phosphine Oxide-Containing, Potent, Orally Active Inhibitor of Anaplastic Lymphoma Kinase.” J. Med. Chem. 59: 4948-4964 (2016); the crystal structure PDB 4vOg and related ligands described in Hennessy, E. J. et al. “Utilization of Structure-Based Design to Identify Novel, Irreversible Inhibitors of EGFR Harboring the T790M Mutation.” ACS. Med. Chem. Lett. 7: 514-519 (2016); the crystal structure PDB 5hg7 and related ligands described in Cheng, H. “Discovery of 1-{(3R,4R)-3-[({5-Chloro-2-[(1-methyl-iH-pyrazol-4-yl)amino]-7H-pyrrolo[2,3-d]pyrimidin-4-yl}oxy)methyl]-4-methoxypyrrolidin-1-yl}prop-2-en-1-one (PF-06459988), a Potent, WT Sparing, Irreversible Inhibitor of T790M-Containing EGFR Mutants.” J. Med. Chem. 59: 2005-2024 (2016); Hao, Y. “Discovery and Structural Optimization of N5-Substituted 6,7-Dioxo-6,7-dihydropteridines as Potent and Selective Epidermal Growth Factor Receptor (EGFR) Inhibitors against L858R/T790M Resistance Mutation.” J. Med. Chem. 59: 7111-7124 (2016); the crystal structure PDB 5ug8, 5ug9, and Sugc and related ligands described in Planken, S. “Discovery of N-((3R,4R)-4-Fluoro-1-(6-((3-methoxy-1-methyl-iH-pyrazol-4-yl)amino)-9-methyl-9H-purin-2-yl)pyrrolidine-3-yl)acrylamide (PF-06747775) through Structure-Based Drug Design: A High Affinity Irreversible Inhibitor Targeting Oncogenic EGFR Mutants with Selectivity over Wild-Type EGFR.” J. Med. Chem. 60: 3002-3019 (2017); the crystal structure PDB 5gnk and related ligands described in Wang, A. “Discovery of (R)-1-(3-(4-Amino-3-(3-chloro-4-(pyridin-2-ylmethoxy)phenyl)-1H-pyrazolo[3,4-d]pyrimidin-1-yl)piperidin-1-yl)prop-2-en-1-one (CHMFL-EGFR-202) as a Novel Irreversible EGFR Mutant Kinase Inhibitor with a Distinct Binding Mode.” J. Med. Chem. 60: 2944-2962 (2017); and, Juchum, M. “Trisubstituted imidazoles with a rigidized hinge binding motif act as single digit nM inhibitors of clinically relevant EGFR L858R/T790M and L858R/T790M/C797S mutants: An example of target hopping.” J. Med. Chem. DOI: 10.1021/acs.jmedchem.7b00178 (2017).

FIG. 8WW-8XX provide non-limiting examples of PAK1 Targeting Ligands wherein R represents exemplary points at which the Linker can be attached. For additional examples and related ligands, see, Rudolph, J. et al. “Chemically Diverse Group I p21-Activated Kinase(PAK) Inhibitors Impart Acute Cardiovascular Toxicity with a Narrow Therapeutic Window.” J. Med Chem. 59, 5520-5541 (2016) and Karpov AS, et al. ACS Med Chem Lett. 22;6(7):776-81 (2015).

FIG. 8YY provides non-limiting examples of PAK4 Targeting Ligands wherein R represents exemplary points at which the Linker can be attached. For additional examples and related ligands, see, Staben ST, et al. J Med Chem. 13;57(3):1033-45 (2014) and Guo, C. et al. “Discovery of pyrroloaminopyrazoles as novel PAK inhibitors” J. Med Chem. 55, 4728-4739 (2012).

FIG. 8ZZ-8AAA provide non-limiting examples of IDO Targeting Ligands wherein R represents exemplary points at which the Linker can be attached. For additional examples and related ligands, see, Yue, E. W.; et al. “Discovery of potent competitive inhibitors of indoleamine 2,3-dioxygenase with in vivo pharmacodynamic activity and efficacy in a mouse melanoma model.” J. Med Chem. 52, 7364-7367 (2009); Tojo, S.; et al. “Crystal structures and structure, and activity relationships of imidazothiazole derivatives as IDO1 inhibitors.” ACS Med Chem. Lett. 5, 1119-1123 (2014); Mautino, M. R. et al. “NLG919, a novel indoleamine-2,3- dioxygenase (IDO)-pathway inhibitor drug candidate for cancer therapy” Abstract 491, AACR 104th Annual Meeting 2013; Apr. 6-10, 2013; Washington, DC; and, WO2012142237 titled “Fused imidazole derivatives useful as IDO inhibitors”.

FIG. 8BBB-8EEE provide non-limiting examples of ERK1 and ERK2 Targeting Ligands wherein R represents exemplary points at which the Linker can be attached. For additional examples and related ligands, see, the crystal structures PDB 5K41 and 5K4J and related ligands described in Blake, J. F. et al. “Discovery of (S)-1-(1-(4-Chloro-3-fluorophenyl)-2-hydroxyethyl)-4-(2-((1-methyl-iH-pyrazol-5-yl)amino)pyrimidin-4-yl)pyridin-2(1H)-one (GDC-0994), an Extracellular Signal-Regulated Kinase 1/2 (ERK1/2) Inhibitor in Early Clinical Development” J. Med. Chem. 59: 5650-5660 (2016); the crystal structure PDB 5BVF and related ligands described in Bagdanoff, J. T. et al. “Tetrahydropyrrolo-diazepenones as inhibitors of ERK2 kinase” Bioorg. Med Chem. Lett. 25, 3788-3792 (2015); the crystal structure PDB 4QYY and related ligands described in Deng, Y.et al. “Discovery of Novel, Dual Mechanism ERK Inhibitors by Affinity Selection Screening of an Inactive Kinase” J Med Chem. 57: 8817-8826 (2014); the crystal structures PDB 5HD4 and 5HD7 and the related ligands described in Jha, S. et al. “Dissecting Therapeutic Resistance to ERK Inhibition” Mol.Cancer Ther. 15: 548-559 (2016); the crystal structure PDB 4XJO and related ligands described in Ren, L. et al. “Discovery of highly potent, selective, and efficacious small molecule inhibitors of ERK1/2.” J. Med Chem. 58: 1976-1991 (2015); the crystal structures PDB 4ZZM, 4ZZN, 4ZZO and related ligands described in Ward, R. A. et al. “Structure-Guided Design of Highly Selective and Potent Covalent Inhibitors of Erkl/2.” J. Med. Chem. 58: 4790 (2015); Burrows, F. et al. “KO-947, a potent ERK inhibitor with robust preclinical single agent activity in MAPK pathway dysregulated tumors” Poster #5168, AACR National Meeting 2017; Bhagwat, S. V. et al. “Discovery of LY3214996, a selective and novel ERK1/2 inhibitor with potent antitumor activities in cancer models with MAPK pathway alterations.” AACR National Meeting 2017; the crystal structures PDB 3FHR and 3FXH and related ligands described in Cheng, R. et al. “High-resolution crystal structure of human Mapkap kinase 3 in complex with a high affinity ligand” Protein Sci. 19: 168-173 (2010); the crystal structures PDB 5NGU, 5NHF, 5NHH, 5NHJ, 5NHL, 5NHO, 5NHP, and 5NHV and related ligands described in Ward, R. A. et al. “Structure-Guided Discovery of Potent and Selective Inhibitors of ERK1/2 from a Modestly Active and Promiscuous Chemical Start Point.” J. Med. Chem. 60, 3438-3450 (2017); the crystal structures PDB 3SHE and 3R1N and related ligands described in Oubrie, A. et al. “Novel ATP competitive MK2 inhibitors with potent biochemical and cell-based activity throughout the series.” Bioorg. Med. Chem. Lett. 22: 613-618 (2012); “Structure-Guided Design of Potent and Selective Pyrimidylpyrrole Inhibitors of Extracellular Signal-Regulated Kinase (ERK) Using Conformational Control” J Med Chem 2009, 52(20), 6362; WO2015051341; “Discovery of a Potent and Selective Oral Inhibitor of ERK1/2 (AZD0364) That Is Efficacious in Both Monotherapy and Combination Therapy in Models of Non-small Cell Lung Cancer (NSCLC)” J Med Chem 2019, 62(24), 11004; and “ERK Inhibitor LY3214996 Targets ERK Pathway-Driven Cancers: A Therapeutic Approach Toward Precision Medicine” Mol Cancer Ther 2020, 19, 325..

FIG. 8FFF-8111 provide non-limiting examples of ABL1 Targeting Ligands wherein R represents exemplary points at which the Linker can be attached. For additional examples and related ligands, see, the crystal structure PDB lfpu and 2e2b and related ligands described in Schindler, T., et al. “Structural mechanism for STI-571 inhibition of abelson tyrosine kinase”, Science 289: 1938-1942 (2000); and Horio, T. et al. “Structural factors contributing to the Abl/Lyn dual inhibitory activity of 3-substituted benzamide derivatives”, Bioorg. Med. Chem. Lett. 17: 2712-2717 (2007); the crystal structures PDB 2hzn and 2hiw and related ligands described in Cowan-Jacob, S.W._et al. “Structural biology contributions to the discovery of drugs to treat chronic myelogenous leukemia”, Acta Crystallog. Sect. D 63: 80-93 (2007) and Okram, B. et al. “A general strategy for creating”, Chem. Biol. 13: 779-786 (2006); the crystal structure PDB 3cs9 and related ligands described in Weisberg, E. et al. “Characterization of AMN107, a selective inhibitor of native and mutant Bcr-Abl”, Cancer Cell 7: 129-14 (2005); the crystal structure PDB 3ik3 and related ligands described in O'Hare, T. et al. “AP24534, a pan-BCR-ABL inhibitor for chronic myeloid leukemia, potently inhibits the T315I mutant and overcomes mutation-based resistance”, Cancer Cell 16: 401-412 (2009); the crystal structure PDB 3mss and related ligands described in Jahnke, W. et al. “Binding or bending: distinction of allosteric Abl kinase agonists from antagonists by an NMIR-based conformational assay”, J. Am. Chem. Soc. 132: 7043-7048 (2010); the crystal structure PDB 3oy3 and related ligands described in Zhou, T. et al. “Structural Mechanism of the Pan-BCR-ABL Inhibitor Ponatinib (AP24534): Lessons for Overcoming Kinase Inhibitor Resistance”, Chem. Biol. Drug Des. 77: 1-11 (2011); the crystal structures PDB 3qri and 3qrk and related ligands described in Chan, W. W. et al. “Conformational Control Inhibition of the BCR-ABL1 Tyrosine Kinase, Including the Gatekeeper T315I Mutant, by the Switch-Control Inhibitor DCC-2036”, Cancer Cell 19: 556-568 (2011); the crystal structure PDB 5hu9 and 2f4j and related ligands described in Liu, F. et al. “Discovery and characterization of a novel potent type II native and mutant BCR-ABL inhibitor (CHMFL-074) for Chronic Myeloid Leukemia (CML)”, Oncotarget 7: 45562-45574 (2016) and Young, M.A. et al. “Structure of the kinase domain of an imatinib-resistant Abl mutant in complex with the Aurora kinase inhibitor VX-680”, Cancer Res. 66: 1007-1014 (2006); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006); and Zhou, T. et al. “Crystal Structure of the T315I Mutant of Abl Kinase”, Chem. Biol. Drug Des. 70: 171-181 (2007); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006) and Zhou, T. et al. “Crystal Structure of the T315I Mutant of Abl Kinase”, Chem. Biol. Drug Des. 70: 171-181 (2007); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006) and Zhou, T. et al. “Crystal Structure of the T315I Mutant of Abl Kinase”, Chem. Biol. DrugDes. 70: 171-181(2007); the crystal structures PDB 3dk3 and 3dk8 and related ligands described in Berkholz, D. S. et al. “Catalytic cycle of human glutathione reductase near 1 A resolution” J Mol. Biol. 382: 371-384 (2008); the crystal structure PDB 3ue4 and related ligands described in Levinson, N.M. et al. “Structural and spectroscopic analysis of the kinase inhibitor bosutinib and an isomer of bosutinib binding to the abl tyrosine kinase domain”, Plos One 7: e29828-e29828 (2012); the crystal structure PDB 4cy8 and related ligands described in Jensen, C. N. et al.“Structures of the Apo and Fad-Bound Forms of 2-Hydroxybiphenyl 3-Monooxygenase (Hbpa) Locate Activity Hotspots Identified by Using Directed Evolution”, Chembiochem 16: 968 (2015); the crystal structure PDB 2hz0 and related ligands described in Cowan-Jacob, S. W. et al. “Structural biology contributions to the discovery of drugs to treat chronic myelogenous leukaemia”, Acta Crystallogr D Biol Crystallogr. 63(Pt 1):80-93 (2007); the crystal structure PDB 3pyy and related ligands described in Yang, J. et al. “Discovery and Characterization of a Cell-Permeable, Small-Molecule c-Abl Kinase Activator that Binds to the Myristoyl Binding Site”, Chem. Biol. 18: 177-186 (2011); and, the crystal structure PDB 5k5v and related ligands described in Kim, M. K., et al. “Structural basis for dual specificity of yeast N-terminal amidase in the N-end rule pathway”, Proc. Natl. Acad. Sci. U.S.A. 113: 12438-12443 (2016).

FIG. 8JJJ provide non-limiting examples of ABL2 Targeting Ligands wherein R represents exemplary points at which the Linker can be attached. For additional examples and related ligands, see, the crystal structure PDB 2xyn and related ligands described in Salah, E. et al. “Crystal Structures of Abl-Related Gene (Abl2) in Complex with Imatinib, Tozasertib (Vx-680), and a Type I Inhibitor of the Triazole Carbothioamide Class”, J. Med. Chem. 54: 2359 (2011); the crystal structure PDB 4xli and related ligands described in Ha, B.H. et al. “Structure of the ABL2/ARG kinase in complex with dasatinib” Acta Crystallogr. Sect.F 71: 443-448 (2015); and the crystal structure PDB 3gvu and related ligands described in Salah, E. et al. “The crystal structure of human ABL2 in complex with Gleevec”, to be published.

FIG. 8KKK-8MMM provide non-limiting examples of AKT1 Targeting Ligands wherein R represents exemplary points at which the Linker can be attached. For additional examples and related ligands, see, Lippa, B. et al. “Synthesis and structure based optimization of novel Akt inhibitors Bioorg. Med. Chem. Lett. 18: 3359-3363 (2008); Freeman-Cook, K. D. et al. “Design of selective, ATP-competitive inhibitors of Akt”, J. Med. Chem. 53: 4615-4622 (2010); Blake, J. F. et al “Discovery of pyrrolopyrimidine inhibitors of Akt”, Bioorg. Med. Chem. Lett. 20: 5607-5612 (2010); Kallan, N.C. et al. “Discovery and SAR of spirochromane Akt inhibitors”, Bioorg. Med. Chem. Lett. 21: 2410-2414 (2011); Lin, K “An ATP-Site On-Off Switch That Restricts Phosphatase Accessibility of Akt”, Sci.Signal. 5: ra37-ra37 (2012); Addie, M. et al. “Discovery of 4-Amino-N-[(1S)-1-(4-chlorophenyl)-3-hydroxypropyl]-1-(7H-pyrrolo[2,3-d]pyrimidin-4-yl)piperidine-4-carboxamide (AZD5363), an Orally Bioavailable, Potent Inhibitor of Akt Kinases”, J. Med. Chem. 56: 2059-2073 (2013); Wu, W.I., et al. “Crystal structure of human AKT1 with an allosteric inhibitor reveals a new mode of kinase inhibition. Plos One 5: 12913-12913 (2010); Ashwell, M.A. et al. “Discovery and optimization of a series of 3-(3-phenyl-3H-imidazo[4,5-b]pyridin-2-yl)pyridin-2-amines: orally bioavailable, selective, and potent ATP-independent Akt inhibitors”, J. Med. Chem. 55: 5291-5310 (2012); and, Lapierre, J. M. et al. “Discovery of 3-(3-(4-(1-Aminocyclobutyl)phenyl)-5-phenyl-3H-imidazo[4,5-b]pyridin-2-yl)pyridin-2-amine (ARQ 092): An Orally Bioavailable, Selective, and Potent Allosteric AKT Inhibitor”, J. Med. Chem. 59: 6455-6469 (2016).

FIG. 8NNN-8OOO provide non-limiting examples of AKT2 Targeting Ligands wherein R represents exemplary points at which the Linker can be attached. For additional examples and related ligands, see, the crystal structured PDB 2jdo and 2jdr and related ligands 10 described in Davies, T.G.et al. “A Structural Comparison of Inhibitor Binding to Pkb, Pka and Pka-Pkb Chimera”, J. Mol. Biol. 367: 882 (2007); the crystal structure PDB 2uw9 and related ligands described in Saxty, G. et al “Identification of Inhibitors of Protein Kinase B Using Fragment-Based Lead Discovery”, J. Med. Chem. 50: 2293-2296 (2007); the crystal structure PDB 2x39 and 2xh5 and related ligands described in Mchardy, T.et al. “Discovery of 4-Amino-1-(7H-Pyrrolo[2,3-D]Pyrimidin-4-Yl)Piperidine-4-Carboxamides as Selective, Orally Active Inhibitors of Protein Kinase B (Akt)”, J. Med. Chem. 53: 2239d (2010); the crystal structure PDB 3d03 and related ligands described in Hadler, K.S. et al. “Substrate-promoted formation of a catalytically competent binuclear center and regulation of reactivity in a glycerophosphodiesterase from Enterobacter aerogenes, J. Am. Chem. Soc. 130: 14129-14138 (2008); and, the crystal structures PDB 3e87, 3e8d and 3e88 and related ligands described in Rouse, M. B. et al. “Aminofurazans as potent inhibitors of AKT kinase” Bioorg. Med. Chem. Lett. 19: 1508-1511 (2009).

FIG. 8PPP provides non-limiting examples of BMX Targeting Ligands wherein R represents exemplary points at which the Linker can be attached. For additional examples and related ligands, see, the crystal structures PDB 3sxr and 3sxr and related ligands described in Muckelbauer, J. et al. “X-ray crystal structure of bone marrow kinase in the x chromosome: a Tec family kinase”, Chem. Biol. Drug Des. 78: 739-748 (2011).

FIG. 8QQQ-8SSS provide non-limiting examples of CSF1R Targeting Ligands wherein R represents exemplary points at which the Linker can be attached. For additional examples and related ligands, see, the crystal structures PDB 2iOv and 2ilm and related ligands described in Schubert, C. et al. “Crystal structure of the tyrosine kinase domain of colony-stimulating factor-1 receptor (cFMS) in complex with two inhibitors”, J. Biol. Chem. 282: 4094-4101 (2007); the crystal structure PDB 3bea and related ligands described in Huang, H. et al. “Design and synthesis of a pyrido[2,3-d]pyrimidin-5-one class of anti-inflammatory FMS inhibitors”, Bioorg. Med. Chem. Lett. 18: 2355-2361 (2008); the crystal structure PDB 3dpk and related ligands described in M.T., McKay, D.B. Overgaard, “Structure of the Elastase of Pseudomonas aeruginosa Complexed with Phosphoramidon”, to be published; the crystal structures PDB 3krj and 3krl and related ligands described in Illig, C. R. et al. “Optimization of a Potent Class of Arylamide Colony-Stimulating Factor-1 Receptor Inhibitors Leading to Anti-inflammatory Clinical Candidate 4-Cyano-N-[2-(1-cyclohexen-1-yl)-4-[1-[(dimethylamino)acetyl]-4-piperidinyl]phenyl]-1H-imidazole-2-carboxamide (JNJ-28312141”, J. Med. Chem. 54: 7860-7883 (2011); the crystal structure PDB 4r7 h and related ligands described in Tap, W. D. et al. “Structure-Guided Blockade of CSF1R Kinase in Tenosynovial Giant-Cell Tumor:, NEnglJMed 373: 428-437 (2015); the crystal structure PDB 31cd and 31coa and related ligands described in Meyers, M. J. et al. “Structure-based drug design enables conversion of a DFG-in binding CSF-1R kinase inhibitor to a DFG-out binding mod”, Bioorg. Med. Chem. Lett. 20: 1543-1547 (2010); the crystal structure PDB 4hw7 and related ligands described in Zhang, C. et al. “Design and pharmacology of a highly specific dual FMS and KIT kinase inhibitor”, Proc. Natl. Acad. Sci. USA 110: 5689-5694 (2013); and, the crystal structure PDB 4r7i and related ligands described in Tap, W. D. et al. “Structure-Guided Blockade of CSF1R Kinase in Tenosynovial Giant-Cell Tumor”, NEngl J Med 373: 428-437 (2015).

FIG. 8TTT provides non-limiting examples of CSK Targeting Ligands wherein R represents exemplary points at which the Linker can be attached. For additional examples and related ligands, see, Levinson, N.M. et al. “Structural basis for the recognition of c-Src by its inactivator Csk”, Cell 134: 124-134 (2008).

FIG. 8UUU-8YYY provide non-limiting examples of DDR1 Targeting Ligands wherein R represents exemplary points at which the Linker can be attached. For additional examples and related ligands, see, the crystal structures PDB 3zos and 4bkj and related ligands described in Canning, P. et al. “Structural Mechanisms Determining Inhibition of the Collagen Receptor Ddrl by Selective and Multi-Targeted Type II Kinase Inhibitors”, J. Mol. Biol. 426: 2457 (2014); the crystal structure PDB 4ckr and related ligands described in Kim, H. et al. “Discovery of a Potent and Selective Ddrl Receptor Tyrosine Kinase Inhibitor”, ACS Chem.Biol. 8: 2145 (2013); the crystal structure PDB 5bvk, 5bvn and 5bvw and related ligands described in Murray, C. W et al. “Fragment-Based Discovery of Potent and Selective DDR1/2 Inhibitors”, ACSMed.Chem.Lett. 6: 798-803 (2015); the crystal structure PDB 5fdp and related ligands described in Wang, Z. et al. “Structure-Based Design of Tetrahydroisoquinoline-7-carboxamides as Selective Discoidin Domain Receptor 1 (DDR1) Inhibitors”, J. Med. Chem. 59: 5911-5916 (2016); and, the crystal structure PDB 5fdx and related ligands described in Bartual, S. G. et al. “Structure of DDR1 receptor tyrosine kinase in complex with D2164 inhibitor at 2.65 Angstroms resolution”, to be published.

FIG. 8ZZZ-8CCCC provide non-limiting examples of EPHA2 Targeting Ligands wherein R represents exemplary points at which the Linker can be attached. For additional examples and related ligands, see, the crystal structures PDB 5i9x, 5i9y, 5ia0 and Sial and related ligands described in Heinzlmeir, S. et al. “Chemical Proteomics and Structural Biology Define EPHA2 Inhibition by Clinical Kinase Drug”, ACS Chem. Biol. 11: 3400-3411 (2016); the crystal structure PDB 5i9z and related ligands described in Heinzlmeir, S. et al. “Crystal Structure of Ephrin A2 (EphA2) Receptor Protein Kinase with danusertib (PHA739358)”, ACS Chem Biol 11 3400-3411 (2016); and, the crystal structures PDB 5ia2, 5ia3, 5ia4, and 5ia5 and related ligands described in Heinzlmeir, S. et al. “Chemical Proteomics and Structural Biology Define EPHA2 Inhibition by Clinical Kinase Drug”, ACS Chem. Biol. 11: 3400-3411 (2016).

FIG. 8DDDD-8FFFF provide non-limiting examples of EPHA3 Targeting Ligands wherein R represents exemplary points at which the Linker can be attached. For additional examples and related ligands, see, the crystal structure PDB 4g2f and related ligands described in Zhao, H. et al. “Discovery of a novel chemotype of tyrosine kinase inhibitors by fragment-based docking and molecular dynamics”, ACSMed. Chem. Lett. 3: 834-838 (2012); the crystal structure PDB 4gk2 and 4gk3 and related ligands described in Lafleur, K. et al. “Optimization of Inhibitors of the Tyrosine Kinase EphB4. 2. Cellular Potency Improvement and Binding Mode Validation by X-ray Crystallography”, J. Med Chem. 56: 84-96 (2013); the crystal structure PDB 4gk3 and related ligands described in Lafleur, K. et al. “Optimization of Inhibitors of the Tyrosine Kinase EphB4. 2. Cellular Potency Improvement and Binding Mode Validation by X-ray Crystallography”, J. Med Chem. 56: 84-96 (2013); the crystal structure PDB 4p4c and 4p5q and related ligands described in Unzue, A. et al. “Pyrrolo[3,2-b]quinoxaline Derivatives as Types I1/2 and II Eph Tyrosine Kinase Inhibitors: Structure-Based Design, Synthesis, and in Vivo Validation”, J Med Chem. 57: 6834-6844 (2014); the crystal structure PDB 4p5z and related ligands described in Unzue, A. et al. “Pyrrolo[3,2-b]quinoxaline Derivatives as Types I1/2 and II Eph Tyrosine Kinase Inhibitors: Structure-Based Design, Synthesis, and in Vivo Validation”, J Med Chem. 57: 6834-6844 (2014); the crystal structure PDB 4twn and related ligands described in Dong, J. et al. “Structural Analysis of the Binding of Type I, I1/2, and II Inhibitors to Eph Tyrosine Kinases”, ACSMed.Chem.Lett. 6: 79-83 (2015); the crystal structure PDB 3dzq and related ligands described in Walker, J.R. “Kinase Domain of Human Ephrin Type-A Receptor 3 (Epha3) in Complex with ALW-II-38-3”, to be published.

FIG. 8GGGG provides non-limiting examples of EPHA4 Targeting Ligands wherein R represents exemplary points at which the Linker can be attached. For additional examples and related ligands, see, the crystal structure PDB 2y60 and related ligands described in Clifton, I. J. et al. “The Crystal Structure of Isopenicillin N Synthase with Delta((L)-Alpha-Aminoadipoyl)-(L)-Cysteinyl-(D)-Methionine Reveals Thioether Coordination to Iron”, Arch. Biochem. Biophys. 516: 103 (2011) and the crystal structure PDB 2xyu and related ligands described in Van Linden, O. P et al. “Fragment Based Lead Discovery of Small Molecule Inhibitors for the Epha4 Receptor Tyrosine Kinase”, Eur. J. Med. Chem. 47: 493 (2012).

FIG. 8HHHH provides non-limiting examples of EPHA7 Targeting Ligands wherein R represents exemplary points at which the Linker can be attached. For additional examples and related ligands, see, the crystal structure PDB 3dko and related ligands described in Walker, J. R. et al. “Kinase domain of human ephrin type-a receptor 7 (epha7) in complex with ALW-II-49-7”, to be published.

FIG. 8IIII-8LLLL provide non-limiting examples of EPHB4 Targeting Ligands wherein R represents exemplary points at which the Linker can be attached. For additional examples and related ligands, see, the crystal structure PDB 2vx1 and related ligands described in Bardelle, C. et al. “Inhibitors of the Tyrosine Kinase Ephb4. Part 2: Structure-Based Discovery and Optimization of 3,5-Bis Substituted Anilinopyrimidines”, Bioorg. Med Chem. Lett. 18: 5717(2008); the crystal structure PDB 2x9f and related ligands described in Bardelle, C. et al. “Inhibitors of the Tyrosine Kinase Ephb4. Part 3: Identification of Non-Benzodioxole-Based Kinase Inhibitors”, Bioorg. Med Chem. Lett. 20: 6242-6245 (2010); the crystal structure PDB 2xvd and related ligands described in Barlaam, B.et al. “Inhibitors of the Tyrosine Kinase Ephb4. Part 4: Discovery and Optimization of a Benzylic Alcohol Series”, Bioorg. Med Chem. Lett. 21: 2207 (2011); the crystal structure PDB 3zew and related ligands described in Overman, R.C.et al. “Completing the Structural Family Portrait of the Human Ephb Tyrosine Kinase Domains”, Protein Sci. 23: 627 (2014); the crystal structure PDB 4aw5 and related ligands described in Kim, M. H. et al. “The Design, Synthesis, and Biological Evaluation of Potent Receptor Tyrosine Kinase Inhibitors”, Bioorg. Med Chem. Lett. 22: 4979 (2012); the crystal structure PDB 4bb4 and related ligands described in Vasbinder, M. M. et al. “Discovery and Optimization of a Novel Series of Potent Mutant B-Raf V600E Selective Kinase Inhibitors” J. Med Chem. 56: 1996.”, (2013); the crystal structures PDB 2vwu, 2vwv and 2vww and related ligands described in Bardelle, C. et al “Inhibitors of the Tyrosine Kinase Ephb4. Part 1: Structure-Based Design and Optimization of a Series of 2,4-Bis-Anilinopyrimidines”, Bioorg. Med. Chem. Lett. 18: 2776-2780 (2008); the crystal structures PDB 2vwx, 2vwy, and 2vwz and related ligands described in Bardelle, C. et al. “Inhibitors of the Tyrosine Kinase Ephb4. Part 2: Structure-Based Discovery and Optimization of 3,5-Bis Substituted Anilinopyrimidines”, Bioorg. Med. Chem. Lett. 18: 5717 (2008); and, the crystal structure PDB 2vxo and related ligands described in Welin, M.et al. “Substrate Specificity and Oligomerization of Human Gmp Synthetas”, J. Mol. Biol. 425: 4323 (2013).

FIG. 8MMMM provides non-limiting examples of ERBB2 Targeting Ligands wherein R represents exemplary points at which the Linker can be attached. For additional examples and related ligands, see, the crystal structure and related ligands described in Aertgeerts, K. et al “Structural Analysis of the Mechanism of Inhibition and Allosteric Activation of the Kinase Domain of HER2 Protein”, J. Biol. Chem. 286: 18756-18765 (2011) and the crystal structure and related ligands described in Ishikawa, T.et al. “Design and Synthesis of Novel Human Epidermal Growth Factor Receptor 2 (HER2)/Epidermal Growth Factor Receptor (EGFR) Dual Inhibitors Bearing a Pyrrolo[3,2-d]pyrimidine Scaffold” J Med. Chem. 54: 8030-8050 (2011).

FIG. 8NNNN provides non-limiting examples of ERBB3 Targeting Ligands wherein R represents exemplary points at which the Linker can be attached. For additional examples and related ligands, see, Littlefield, P.et al. “An ATP-Competitive Inhibitor Modulates the Allosteric Function of the HER3 Pseudokinase”, Chem. Biol. 21: 453-458 (2014).

FIG. 8OOOO provides non-limiting examples ERBB4 Targeting Ligands wherein R represents exemplary points at which the Linker can be attached. For additional examples and related ligands, see, Qiu, C. et al. “Mechanism of Activation and Inhibition of the HER4/ErbB4 Kinase”, Structure 16: 460-467 (2008) and Wood, E. R. et al. “6-Ethynylthieno[3,2-d]- and 6-ethynylthieno[2,3-d]pyrimidin-4-anilines as tunable covalent modifiers of ErbB kinases”, Proc. Nat!. Acad. Sci. Usa 105: 2773-2778 (2008).

FIG. 8PPPP-8QQQQ provide non-limiting examples of FES Targeting Ligands wherein R represents exemplary points at which the Linker can be attached. For additional examples and related ligands, see, Filippakopoulos, P. et al “Structural Coupling of SH2-Kinase Domains Links Fes and Abl Substrate Recognition and Kinase Activation.” Cell 134: 793-803 (2008) and Hellwig, S. et al. “Small-Molecule Inhibitors of the c-Fes Protein-Tyrosine Kinase”, Chem. Biol. 19: 529-540 (2012).

FIG. 8RRRR provides non-limiting examples of FYN Targeting Ligands wherein R represents exemplary points at which the Linker can be attached. For additional examples and related ligands, see, Kinoshita, T. et. al. “Structure of human Fyn kinase domain complexed with staurosporine”, Biochem. Biophys. Res. Commun. 346: 840-844 (2006).

FIG. 8SSSS-8VVVV provide non-limiting examples of GSG2 (Haspin) Targeting Ligands wherein R represents exemplary points at which the Linker can be attached. For additional examples and related ligands, see, the crystal structures PDB 3e7v, PDB 3f2n, 3fmd and related ligands described in Filippakopoulos, P. et al. “Crystal Structure of Human Haspin with a pyrazolo-pyrimidine ligand”, to be published; the crystal structure PDB 3iq7 and related ligands described in Eswaran, J. et al. “Structure and functional characterization of the atypical human kinase haspin”, Proc. Natl. Acad. Sci. USA 106: 20198-20203 (2009); and, the crystal structure PDB 4qtc and related ligands described in Chaikuad, A. et al. “A unique inhibitor binding site in ERK1/2 is associated with slow binding kinetics”, Nat. Chem. Biol. 10: 853-860 (2014).

FIG. 8WWWW-8AAAAA provide non-limiting examples of HCK Targeting Ligands wherein R represents exemplary points at which the Linker can be attached. For additional examples and related ligands, see, the crystal structure PDB lqcf and related ligands described in Schindler, T. et al. “Crystal structure of Hck in complex with a Src family-selective tyrosine kinase inhibitor”, Mol. Cell 3: 639-648 (1999); the crystal structure PDB 2c0i and 2cOt and related ligands described in Burchat, A. et al. “Discovery of A-770041, a Src-Family Selective Orally Active Lck Inhibitor that Prevents Organ Allograft Rejection”, Bioorg. Med. Chem. Lett. 16: 118 (2006); the crystal structure PDB 2hk5 and related ligands described in Sabat, M.et al. “The development of 2-benzimidazole substituted pyrimidine based inhibitors of lymphocyte specific kinase (Lck)”, Bioorg. Med. Chem. Lett. 16: 5973-5977 (2006); the crystal structures PDB 3vry, 3vs3, 3vs6, and 3vs7 and related ligands described in Saito, Y. et al. “A Pyrrolo-Pyrimidine Derivative Targets Human Primary AML Stem Cells in Vivo”, Sci Transl Med 5: 181ra52-181ra52 (2013); and, the crystal structure PDB 4lud and related ligands described in Parker, L. J. et al “Kinase crystal identification and ATP-competitive inhibitor screening using the fluorescent ligand SKF86002”,. Acta Crystallogr.,Sect.D 70: 392-404 (2014).

FIG. 8BBBBB-8FFFFF provide non-limiting examples of IGFIR Targeting Ligands wherein R represents exemplary points at which the Linker can be attached. For additional examples and related ligands, see, the crystal structure PDB 2oj9 and related ligands described in Velaparthi, U. et al. “Discovery and initial SAR of 3-(1H-benzo[d]imidazol-2-yl)pyridin-2(1H)-ones as inhibitors of insulin-like growth factor 1-receptor (IGF-1R)”, Bioorg. Med. Chem. Lett. 17: 2317-2321 (2007); the crystal structure PDB 3i81 and related ligands described in Wittman, M.D. et al. “Discovery of a 2,4-disubstituted pyrrolo[1,2-f][1,2,4]triazine inhibitor (BMS-754807) of insulin-like growth factor receptor (IGF-1R) kinase in clinical development.”, J. Med. Chem. 52: 7360-7363 (2009); the crystal structure PDB 3nw5 and related ligands described in Sampognaro, A. J. et al. “Proline isosteres in a series of 2,4-disubstituted pyrrolo[1,2-f][1,2,4]triazine inhibitors of IGF-1R kinase and IR kinase”, Bioorg. Med. Chem. Lett. 20: 5027-5030 (2010); the crystal structure PDB 3qqu and related ligands described in Buchanan, J. L. et al. “Discovery of 2,4-bis-arylamino-1,3-pyrimidines as insulin-like growth factor-1 receptor (IGF-1R) inhibitors”, Bioorg. Med. Chem. Lett. 21: 2394-2399 (2011); the crystal structure PDB 4d2r and related ligands described in Kettle, J. G. et al. “Discovery and Optimization of a Novel Series of DyrklB Kinase Inhibitors to Explore a Mek Resistance Hypothesis”. J. Med. Chem. 58: 2834 (2015); the crystal structure PDB 3fxq and related ligands described in Monferrer, D. et al. “Structural studies on the full-length LysR-type regulator TsaR from Comamonas testosteroni T-2 reveal a novel open conformation of the tetrameric LTTR fold”, Mol. Microbiol. 75: 1199-1214 (2010); the crystal structure PDB 5fxs and related ligands described in Degorce, S. et al. “Discovery of Azd9362, a Potent Selective Orally Bioavailable and Efficacious Novel Inhibitor of Igf-R1”, to be published; the crystal structure PDB 2zm3 and related ligands described in Mayer, S.C.et al. “Lead identification to generate isoquinolinedione inhibitors of insulin-like growth factor receptor (IGF-1R) for potential use in cancer treatment”, Bioorg. Med. Chem. Lett. 18: 3641-3645 (2008); the crystal structure PDB 3f5p and related ligands described in “Lead identification to generate 3-cyanoquinoline inhibitors of insulin-like growth factor receptor (IGF-1R) for potential use in cancer treatment” Bioorg. Med. Chem. Lett. 19: 62-66 (2009); the crystal structure PDB 31vp and related ligands described in Nemecek, C. et al. “Design of Potent IGF1-R Inhibitors Related to Bis-azaindoles” Chem. Biol. Drug Des. 76: 100-106 (2010); the crystal structure PDB 3o23 and related ligands described in Lesuisse, D. et al. “Discovery of the first non-ATP competitive IGF-1R kinase inhibitors: Advantages in comparison with competitive inhibitors”, Bioorg. Med. Chem. Lett. 21: 2224-2228 (2011); the crystal structure PDB 3d94 and related ligands described in Wu, J. et al. “Small-molecule inhibition and activation-loop trans-phosphorylation of the IGF1 receptor”, Embo J. 27: 1985-1994 (2008); and, the crystal structure PDB 5hzn and related ligands described in Stauffer, F.et al. “Identification of a 5-[3-phenyl-(2-cyclic-ether)-methylether]-4-aminopyrrolo[2,3-d]pyrimidine series of IGF-1R inhibitors”, Bioorg. Med. Chem. Lett. 26: 2065-2067 (2016).

FIG. 8GGGGG-8JJJJJ provide non-limiting examples of INSR Targeting Ligands wherein R represents exemplary points at which the Linker can be attached. For additional examples and related ligands, see, the crystal structure PDB 2z8c and related ligands described in Katayama, N. et al. “Identification of a key element for hydrogen-bonding patterns between protein kinases and their inhibitors”, Proteins 73: 795-801 (2008); the crystal structure PDB 3ekk and related ligands described in Chamberlain, S.D.et al. “Discovery of 4,6-bis-anilino-1H-pyrrolo[2,3-d]pyrimidines: Potent inhibitors of the IGF-1R receptor tyrosine kinase”, (2009) Bioorg. Med. Chem. Lett. 19: 469-473; the crystal structure PDB 3ekn and related ligands described in Chamberlain, S.D. et al. “Optimization of 4,6-bis-anilino-1H-pyrrolo[2,3-d]pyrimidine IGF-1R tyrosine kinase inhibitors towards INK selectivity”, Bioorg. Med Chem. Lett. 19: 360-364 (2009); the crystal structure PDB 5els and related ligands described in Sanderson, M. P. et al. “BI 885578, a Novel IGF1R/INSR Tyrosine Kinase Inhibitor with Pharmacokinetic Properties That Dissociate Antitumor Efficacy and Perturbation of Glucose Homeostasis” Mol. Cancer Ther. 14: 2762-2772 “, (2015); the crystal structure PDB 3eta and related ligands described in Patnaik, S. et al. “Discovery of 3,5-disubstituted-1H-pyrrolo[2,3-b]pyridines as potent inhibitors of the insulin-like growth factor-1 receptor (IGF-1R) tyrosine kinase”, Bioorg. Med. Chem. Lett. 19: 3136-3140 (2009); the crystal structure PDB 5hhw and related ligands described in Stauffer, F.et al. “Identification of a 5-[3-phenyl-(2-cyclic-ether)-methylether]-4-aminopyrrolo[2,3-d]pyrimidine series of IGF-1R inhibitors”, Bioorg. Med. Chem. Lett. 26: 2065-2067 (2016); and, the crystal structure PDB 4ibm and related ligands described in Anastassiadis, T. et al. “A highly selective dual insulin receptor (IR)/insulin-like growth factor 1 receptor (IGF-1R) inhibitor derived from an extracellular signal-regulated kinase (ERK) inhibitor”, J. Biol. Chem. 288: 28068-28077 (2013).

FIG. 8KKKKK-8PPPPP provide non-limiting examples of HBV Targeting Ligands wherein R represents exemplary points at which the Linker can be attached, Y is methyl or isopropyl, and X is N or C. For additional examples and related ligands, see, Weber, O.; et al. “Inhibition of human hepatitis B virus (HBV) by a novel non-nucleosidic compound in a transgenic mouse model.” Antiviral Res.54, 69-78 (2002); Deres, K.; et al. “Inhibition of hepatitis B virus replication by drug-induced depletion of nucleocapsids.” Science, 299, 893-896 (2003); Stray, S. J.; Zlotnick, A. “BAY 41-4109 has multiple effects on Hepatitis B virus capsid assembly.” J. Mol. Recognit. 19, 542-548 (2006); Stray, S. J.; et al. “heteroaryldihydropyrimidine activates and can misdirect hepatitis B virus capsid assembly.” Proc. Natl. Acad. Sci. U.S.A, 102, 8138-8143 (2005); Guan, H.; et al. “The novel compound Z060228 inhibits assembly of the HBV capsid.” Life Sci. 133, 1-7 (2015); Wang, X. Y.; et al. “In vitro inhibition of HBV replication by a novel compound, GLS4, and its efficacy against adefovir-dipivoxil-resistant HBV mutations.” Antiviral Ther. 17, 793-803 (2012); Klumpp, K.; et al. “High-resolution crystal structure of a hepatitis B virus replication inhibitor bound to the viral core protein.” 112, 15196-15201 (2015); Qiu, Z.; et al. “Design and synthesis of orally bioavailable 4-methyl heteroaryldihydropyrimidine based hepatitis B virus (HBV) capsid inhibitors.” J. Med. Chem. 59, 7651-7666 (2016); Zhu, X.; et al. “2,4-Diaryl-4,6,7,8-tetrahydroquinazolin-5(1H)-one derivatives as anti-HBV agents targeting at capsid assembly.” Bioorg. Med. Chem. Lett. 20, 299-301 (2010); Campagna, M. R.; et al. “Sulfamoylbenzamide derivatives inhibit the assembly of hepatitis B virus nucleocapsids.” J. Virol. 87, 6931-6942 (2013); Campagna, M. R.; et al. “Sulfamoylbenzamide derivatives inhibit the assembly of hepatitis B virus nucleocapsids.” J. Virol. 87, 6931-6942 (2013); WO 2013096744 A1 titled “Hepatitis B antiviral agents”; WO 2015138895 titled “Hepatitis B core protein allosteric modulators”; Wang, Y. J.; et al. “A novel pyridazinone derivative inhibits hepatitis B virus replication by inducing genome-free capsid formation.” Antimicrob. Agents Chemother. 59, 7061-7072 (2015); WO 2014033167 titled “Fused bicyclic sulfamoyl derivatives for the treatment of hepatitis”; U.S. 20150132258 titled “Azepane derivatives and methods of treating hepatitis B infections”; and, WO 2015057945 “Hepatitis B viral assembly effector”.

FIG. 9 is a dendrogram of the human bromodomain family of proteins organized into eight sub families, which are involved in epigenetic signaling and chromatin biology. Any of the proteins of the bromodomain family in FIG. 9 can be selected as a Target Protein according to the present invention.

FIG. 10A and FIG. 10B provide non-limiting examples of CBP and/or P300 Targeting Ligands, wherein R represents exemplary points at which the Linker can be attached. For example additional examples of Targeting Ligands see “GNE-781, A Highly Advanced Potent and Selective Bromodomain Inhibitor of Cyclic Adenosine Monophosphate Response Element Binding Protein, Binding Protein (CBP)” J Med Chem 2017, 60(22), 9162; CCS-1477, WO2018073586; FT-7051, and WO2019055869.

FIGS. 11A and 11B provide non-limiting examples of BRD9 Targeting Ligands wherein R is the point at which the Linker is attached. For additional examples see: “Structure-Based Design of an in Vivo Active Selective BRD9 Inhibitor” J Med Chem 2016, 59(10), 4462; WO2016139361.

FIG. 12A-12C provide non-limiting examples of CBL-B Targeting Ligands, wherein R represents exemplary points at which the Linker can be attached. For additional examples, see W0201914800).

FIG. 13 provides non-limiting examples of ERK Targeting Ligands wherein R is the point at which the Linker is attached. For additional examples see: Arnov A.M. et al. “Structure-Guided Design of Potent and Selective Pyrimidylpyrrole Inhibitors of Extracellular Signal-Regulated Kinase (ERK) Using Conformational Control” J Med Chem 2009, 52(20), 6362; WO2015051341; Ward R.A. et al. “Discovery of a Potent and Selective Oral Inhibitor of ERK1/2 (AZD0364) That Is Efficacious in Both Monotherapy and Combination Therapy in Models of Nonsmall Cell Lung Cancer (NSCLC)” J Med Chem 2019, 62(24), 11004; Bhagwat S.V. et al. “ERK Inhibitor LY3214996 Targets ERK Pathway-Driven Cancers: A Therapeutic Approach Toward Precision Medicine” Mol Cancer Ther 2020, 19, 325.

FIG. 14A-14C provide non-limiting examples of WDR5 Targeting Ligands, wherein R represents exemplary points at which the Linker can be attached. For additional examples see “Structure-Based Optimization of a Small Molecule Antagonist of the Interaction Between WD Repeat-Containing Protein 5 (WDR5) and Mixed-Lineage Leukemia 1 (MLL1)” J Med Chem 2016, 59(6), 2478; WO2017147700; “Displacement of WDR5 from Chromatin by a WIN Site Inhibitor with Picomolar Affinity” Cell Rep 2019, 26(11), 2916; “Discovery and Optimization of Salicylic Acid-Derived Sulfonamide Inhibitors of the WD Repeat-Containing Protein 5-MYC Protein-Protein Interaction” J Med Chem 2019, 62(24), 11232).

FIG. 15 provides non-limiting examples of NSP3 Targeting Ligands, wherein R represents exemplary points at which the Linker can be attached. For additional examples see: “Severe Acute Respiratory Syndrome Coronavirus Papain-like Novel Protease Inhibitors: Design, Synthesis, Protein-Ligand X-ray Structure and Biological Evaluation”, J Med Chem 2010, 53, 4968; “X-ray Structural and Biological Evaluation of a Series of Potent and Highly Selective Inhibitors of Human Coronavirus Papain-like Proteases”, J Med Chem 2014, 57, 2393).

FIG. 16 provides non-limiting examples of RET Targeting Ligands, wherein R represents exemplary points at which the Linker can be attached. For additional examples see: Pralsetinib “Precision Targeted Therapy with BLU-667 for RET-Driven Cancers” Cancer Discovery, 2018, 8(7), 836; Selpercatinib, WO2018071447; “A Pyrazolo[3,4-d]pyrimidin-4-amine Derivative Containing an Isoxazole Moiety Is a Selective and Potent Inhibitor of RET Gatekeeper Mutants” J Med Chem, 2016, 59, 358).

FIG. 17A-17C provide non-limiting examples of CTNNB1 Targeting Ligands wherein R is the point at which the Linker is attached. For additional examples see: “Direct Targeting of b-Catenin by a Small Molecule Stimulates Proteasomal Degradation and Suppresses Oncogenic Wnt/b-Catenin Signaling” Cell Rep 2016, 16(1), 28 “Rational Design of Small-Molecule Inhibitors for O-Catenin/T-Cell Factor Protein-Protein Interactions by Bioisostere Replacement” ACS Chem Biol 2013, 8, 524, and“Allosteric inhibitor of 0-catenin selectively targets oncogenic Wnt signaling in colon cancer” Sci Rep 2020, 10, 8096.

FIG. 18A-18C provide non-limiting examples of IRAK4 Targeting Ligands, wherein R represents exemplary points at which the Linker can be attached. For additional examples and related ligands see crystal structures PDB 6UYA, 4YP8, 5UIU, and 6F3I in the respective references (Rajapaksa N.S. et al. “Discovery of Potent Benzolactam IRAK4 Inhibitors with Robust in Vivo Activity.” ACS Med. Chem. Lett. 11: 327-333 (2020); McElroy W.T. et al. “Potent and Selective Amidopyrazole Inhibitors of IRAK4 That Are Efficacious in a Rodent Model of Inflammation.” ACS Med. Chem. Lett. 6: 677-682 (2015); Nunes J. et al. “Targeting IRAK4 for Degradation with PROTACs” ACS Med. Chem. Lett. 10: 1081-1085 (2019); 4); Degorce S. L. et al. “Optimization of permeability in a series of pyrrolotriazine inhibitors of IRAK4”. Bioorg. Med. Chem. 26: 913-924 (2018); WO2019099926 and WO2019133531.

FIG. 19A-19D provide non-limiting examples of FGFR2 and FGFR3 Targeting Ligands wherein R is the point at which the Linker is attached. For additional examples see: “Structure-based drug design of 1,3,5-triazine and pyrimidine derivatives as novel FGFR3 inhibitors with high selectivity over VEGFR2” BioorgMed Chem 2020, 28, 115453.

FIG. 20A-20D provide non-limiting examples of SMARCA2 Targeting Ligands wherein R is the point at which the Linker is attached. For additional examples see: WO2020023657, US20200038378, WO2020010227, WO2020078933, WO2019207538, WO2016138114, “Discovery of Orally Active Inhibitors of Brahma Homolog (BRM)/SMARCA2 ATPase Activity for the Treatment of Brahma Related Gene 1 (BRG1)/SMARCA4-Mutant Cancers” J Med Chem 2018, 61, 10155; 2) WO2020035779.

FIG. 21A-21J provide non-limiting examples of NRAS Targeting Ligands, wherein R represents exemplary points at which the Linker can be attached. For additional examples, see “Small-molecule Ligands Bing to a Distinct Pocket in Ras and Inhibit SOS-Mediated Nucleotide Exchange Activity” PNAS 2012 109 (14) 5299-5304; the crystal structure PDB 4EPY. (“Discovery of Small Molecules that Bind to K-Ras and Inhibit Sos-Mediated Activation” Angew. Chem. Int. Ed 2012, 51, 6140-6143); the crystal structure PDB 6GQY, 6GQT, (“Structure-based development of new RAS-effector inhibitors from a combination of active and inactive RAS-binding compounds” 2019 PNAS 116 (7), 2545-2550); the crystal structure PDB 6FA4, 1HE8, (“Small molecule inhibitors of RAS-effector protein interactions derived using an intracellular antibody fragment” 2018 Nature Communications 9(1), 3169); and “Discovery of High-Affinity Noncovalent Allosteric KRAS Inhibitors That Disrupt Effector Binding” ACS Omega 2019, 4, 2921-2930.

FIG. 22 provides a non-limiting example of an ADAR Targeting Ligand, wherein R represents exemplary points at which the Linker can be attached. For additional examples, see the crystal structure PDB 6VFF, (Thuy-Boun, A. S., et al, Nucleic Acids Res, 2020, 48, 7958-7972); and the crystal structures PDB 5HP2, 5HP3, 5ED1, 5ED2 (Mathews, M. M, et al. Nat Struct Mol Biol., 2016, 23, 426-433).

FIG. 23 provides non-limiting examples of NSD2 or WHSC1 Targeting Ligands, wherein R represents exemplary points at which the Linker can be attached. For additional examples, see the crystal structure PDB 6XCG (Zhou, M. Q, et al. “Histone-lysine N-methyltransferase NSD2-PWWP1 with compound UNC6934”, to be published); the crystal structure PDB 6UE6 (Liu, Y et al. “PWWP1 domain of NSD2 in complex with MR837”,to be published); the crystal structure PDB 5LSS, 5LSU, 5LSX, 5LSY, 5LSZ, 5LT6,5LT7, 5LT8 (Tisi, D., et al, “Structure of the Epigenetic Oncogene MMSET and Inhibition by N-Alkyl Sinefungin Derivatives.”, ACS Chem Biol., 2016, 11: 3093-3105).

FIG. 24 provides non-limiting example of PI3KCA Targeting Ligands, wherein R represents exemplary points at which the Linker can be attached. For additional examples, see the crystal structure PDB 3HHM, 3HIZ (Mandelker, D., et al. “A frequent kinase domain mutation that changes the interaction between PI3K{alpha}and the membrane.”, Proc Natl Acad Sci USA., 2009, 106: 16996-17001).

FIG. 25 provides a non-limiting example of a RIT1 Targeting Ligand, wherein R represents exemplary points at which the Linker can be attached. For additional examples, see the crystal structure PDB 4KLZ (Shah, D. M., et al. “Inhibition of Small GTPases by Stabilization of the GDP Complex, a Novel Approach applied to Rit1, a Target for Rheumatoid Arthritis”, to be published).

FIG. 26 provides non-limiting examples of WRN Targeting Ligands, wherein R represents exemplary points at which the Linker can be attached. For additional examples, see the crystal structure PDB 2FC0 (Perry, J. J., et al. “WRN exonuclease structure and molecular mechanism imply an editing role in DNA end processing.“, Nat Struct Mol Biol., 2006, 13: 414-422); and the crystal structure PDB 6YHR (Newman, J. A., et al. “Crystal structure of Werner syndrome helicase”, to be published).

FIG. 27 provides non-limiting examples of ALK-fusion Targeting Ligands, for example EML4-ALK or NMP-ALK, wherein R represents exemplary points at which the Linker can be attached. For additional examples, see the crystal structure PDB 4CGB, 4CGC (Richards, M. W., et al. “Microtubule Association of Eml Proteins and the Eml4-Alk Variant 3 Oncoprotein Require an N-Terminal Trimerization Domain”, Biochem J., 2015, 467: 529); the crystal structure PDB 3AOX (Sakamoto, H., et al. “CH5424802, a selective ALK inhibitor capable of blocking the resistant gatekeeper mutant”, Cancer Cell, 2011, 19: 679-690); the crystal structure PDB 6MX8 (Huang, W. S., et al. “Discovery of Brigatinib (AP26113), a Phosphine Oxide-Containing, Potent, Orally Active Inhibitor of Anaplastic Lymphoma Kinase”, J Med Chem., 2016, 59: 4948-4964); 4Z55 (Michellys, P. Y., et al. “Design and synthesis of novel selective anaplastic lymphoma kinase inhibitors.”, Bioorg Med Chem Lett., 2016, 26: 1090-1096); and the crystal structures PDB 4FOB, 4FOC, 4FOD (Lewis, R. T., et al, “The Discovery and Optimization of a Novel Class of Potent, Selective, and Orally Bioavailable Anaplastic Lymphoma Kinase (ALK) Inhibitors with Potential Utility for the Treatment of Cancer.”, J Med Chem., 2012, 55: 6523-6540).

FIG. 28 provides non-limiting examples of BAP1 Targeting Ligands, wherein R represents exemplary points at which the Linker can be attached. For additional examples, see the crystal structure PDB 2W12, 2W13, 2W14, 2W15 (Lingott, T. J. et al. “High-Resolution Crystal Structure of the Snake Venom Metalloproteinase Bap1 Complexed with a Peptidomimetic: Insight into Inhibitor Binding”, Biochemistry, 2009, 48: 6166).

FIG. 29 provides non-limiting examples of EPAS1 or HIF2a Targeting Ligands, wherein R represents exemplary points at which the Linker can be attached. For additional examples, see the crystal structure PDB 5UFP (Cho, H., et al. “On-target efficacy of a HIF-2 alpha antagonist in preclinical kidney cancer models.”, Nature, 2016, 539: 107-111); the crystal structure PDB 6D09 Du, X, (“Crystal structure of PT1940 bound to HIF2a-B*:ARNT-B* complex”, to be published); the crystal structure PDB 5TBM (Wallace, E. M., et al.” A Small-Molecule Antagonist of HIF2 alpha Is Efficacious in Preclinical Models of Renal Cell Carcinoma.”, Cancer Res., 2016, 76: 5491-5500); and the crystal structure PDB 6E3S, 6E3T, 6E3U (Wu, D., et al. “Bidirectional modulation of HIF-2 activity through chemical ligands.”, Nat Chem Biol., 2019, 15: 367-376).

FIG. 30A and FIG. 30B provide non-limiting examples of GRB2 Targeting Ligands, wherein R represents exemplary points at which the Linker can be attached. For additional examples, see the crystal structure PDB 1CJ1 (Furet, P., et al. “Structure-based design, synthesis, and X-ray crystallography of a high-affinity antagonist of the Grb2-SH2 domain containing an asparagine mimetic”, J Med Chem., 1999, 42: 2358-2363); the crystal structure PDB 2AOA, 2AOB (Phan, J., et al. “Crystal Structures of a High-affinity Macrocyclic Peptide Mimetic in Complex with the Grb2 SH2 Domain”, J Mol Biol., 2005, 353: 104-115); the crystal structure PDB 3KFJ, 3IN7, 3IMJ, 3IMD, 3IN8 (Delorbe, J. E., et al. “Thermodynamic and Structural Effects of Conformational Constraints in Protein-Ligand Interactions. Entropic Paradoxy Associated with Ligand Preorganization.”, J Am Chem Soc., 2009, 131: 16758-16770); the crystal structure PDB 2HUW, 3C71 (Benfield, A. P., et al. “Ligand Preorganization May Be Accompanied by Entropic Penalties in Protein-Ligand Interactions.”, Angew Chem IntEd Engl., 2006, 45: 6830-6835); and the crystal structure PDB 1X0N (Ogura, K et al. “NMR structure of growth factor receptor binding protein SH2 domain complexed with the inhibitor”, to be published).

FIG. 31 provides non-limiting examples of KMT2D or MLL2/MLL4Targeting Ligands, wherein R represents exemplary points at which the Linker can be attached. For additional examples, see the crystal structure PDB 7BRE (Li, Y., et al. “Crystal Structure of MLL2 Complex Guides the Identification of a Methylation Site on P53 Catalyzed by KMT2 Family Methyltransferases.”, Structure, 2020); the crystal structure PDB 4ZAP (Zhang, Y., et al. “Evolving Catalytic Properties of the MLL Family SET Domain.”, Structure, 2015, 23: 1921-1933); the crystal structure PDB 6KIZ (Xue, H., et al. “Structural basis of nucleosome recognition and modification by MLL methyltransferases.”, Nature, 2019, 573: 445-449); and the crystal structures PDB 3UVK (Zhang, P., et al. “The plasticity of WDR5 peptide-binding cleft enables the binding of the SETl family of histone methyltransferases.”, Nucleic Acids Res., 2012, 40: 4237-4246).

FIG. 32 provides non-limiting examples of MLLT1 or ENL Targeting Ligands, wherein R represents exemplary points at which the Linker can be attached. For additional examples, see the crystal structure PDB 6HTO, 6HT1 (Moustakin, M. et al. “Discovery of an MLLT1/3 YEATS Domain Chemical Probe”, Angew Chem Int Ed Engl., 2018, 57: 16302-16307); the crystal structures PDB 6T1I, 6T1J, 6TIL,6T1M, 6T1N, 6T10 (Ni, X., et al. “Structural Insights into Interaction Mechanisms of Alternative Piperazine-urea YEATS Domain Binders in MLLT1”, ACS Med Chem Lett., 2019, 10: 1661-1666); and the crystal structures PDB 6HPW, 6HPY, 6HPX,6HPZ (Heidenreich, D., et al. “Structure-Based Approach toward Identification of Inhibitory Fragments for Eleven-Nineteen-Leukemia Protein (ENL)”, J Med Chem., 2018, 61: 10929-10934).

FIG. 33 provides non-limiting examples of NSD3 Targeting Ligands, wherein R represents exemplary points at which the Linker can be attached. For additional examples, see the crystal structure PDB 6G24, 6G25, 6G29, 6G2B, 6G2C, 6G2E, 6G2F, 6G20, 6G3T (Bottcher, J., et al. “Fragment-based discovery of a chemical probe for the PWWP1 domain of NSD3”, Nat Chem Biol., 2019, 15: 822-829); the crystal structure PDB 5UPD (Tempel, W., et al. “Methyltransferase domain of human Wolf-Hirschhorn Syndrome Candidate 1-Like protein 1 (WHSCIL1)”, to be published); and the crystal structure PDB 6CEN (Morrison, M. J., et al. “Identification of a peptide inhibitor for the histone methyltransferase WHSC1”, PLoS One, 2018, 13: e0197082-e0197082).

FIG. 34 provides non-limiting examples of PPM1D or WIP1 Targeting Ligands, wherein R represents exemplary points at which the Linker can be attached. For additional examples, see the crystal structure PDB 3UYH, ADA3, 4DAQ (Micco, M., et al. “Structure-based design and evaluation of naphthalene diimide g-quadruplex ligands as telomere targeting agents in pancreatic cancer cells”, J Med Chem., 2013, 56: 2959-2974).

FIG. 35A-35B provide non-limiting examples of SOS1 Targeting Ligands, wherein R represents exemplary points at which the Linker can be attached. For additional examples, see the crystal structure PDB 5OVE, 5OVF, 5OVG, 5OVH, 5OVI, (Hillig, R. C., et al. “Discovery of potent SOS1 inhibitors that block RAS activation via disruption of the RAS-SOS1 interaction”, Proc Natl Acad Sci USA., 2019, 116: 2551-2560); the crystal structure PDB 6F08 (Ballone, A., et al. “Structural characterization of 14-3-3 zeta in complex with the human Son of sevenless homolog 1 (SOS1)”, J Struct Biol., 2018, 202: 210-215); the crystal structure PDB 6D5E, 6D5G, 6D5H, 6D5J, 6D5L, 6D5M, 6D5V, 6D5W, 6D55, 6D59, (Hodges, T. R. et al. “Discovery and Structure-Based Optimization of Benzimidazole-Derived Activators of SOS1-Mediated Nucleotide Exchange on RAS”, J Med Chem., 2018, 61: 8875-8894); the crystal structure PDB 6SCM, 6SFR (Kessler, D., et al.“SOS1 in Complex with Inhibitor BI-3406”, to be published); the crystal structure PDB 6V94, 6V9J, 6V9L, 6V9M, 6V9N (Sarkar, D., et al. “Discovery of Sulfonamide-Derived Agonists of SOS1-Mediated Nucleotide Exchange on RAS Using Fragment-Based Methods.”, J Med Chem., 2020, 63: 8325-8337).

FIG. 36A provides non-limiting examples of TBXT or Brachyury Targeting Ligands, wherein R represents exemplary points at which the Linker can be attached. For additional examples, see the crystal structure PDB 5QS6, 5QSC, 5QSE, 5QSF, 5QRW, (Newman, J. A., et al. “PanDDA analysis group deposition”, to be published); and the crystal structure PBD 6ZU8 (Newman, J. A., et al. “Crystal structure of human Brachyury G177D variant in complex with Afatinib”, to be published).

FIG. 37A-37C provide non-limiting examples of USP7 Targeting Ligands, wherein R represents exemplary points at which the Linker can be attached. For additional examples, see the crystal structure PDB 5UQV, 5UQX (Kategaya, L., et al. “USP7 small-molecule inhibitors interfere with ubiquitin binding”, Nature, 2017, 550: 534-538); the crystal structures PDB 6VN2, 6VN3, 6VN4, 6VN5, 6VN6 (Leger, P. R., et al. “Discovery of Potent, Selective, and Orally Bioavailable Inhibitors of USP7 with In Vivo Antitumor Activity.”, J Med Chem., 2020, 63: 5398-5420); and the crystal structures PDB 5N9R, 5N9T (Gavory, G., et al. “Discovery and characterization of highly potent and selective allosteric USP7 inhibitors.”, Nat Chem Biol., 2018, 14: 118-125); and the crystal structure PDB 5NGE, 5NGF (Turnbull, A. P., et al. “Molecular basis of USP7 inhibition by selective small-molecule inhibitors”, Nature, 2017, 550: 481-486).

FIG. 38 provides non-limiting examples of BKV and JCV Targeting Ligands, wherein R represents exemplary points at which the Linker can be attached. For additional examples, see the crystal structure PDB 5J4V, 5J4Y (Bonafoux, D., et al. “Fragment-Based Discovery of Dual JC Virus and BK Virus Helicase Inhibitors.”, J Med Chem., 2016, 59: 7138-7151).

FIG. 39 provides non-limiting examples of CKla (Casein kinase 1 alpha) Targeting Ligands, wherein R represents exemplary points at which the Linker can be attached. For additional examples, see the crystal structure PDB 5ML5, 5MQV (Halekotte, J., et al. “Optimized 4,5-Diarylimidazoles as Potent/Selective Inhibitors of Protein Kinase CK1 delta and Their Structural Relation to p38 alpha MAPK.”, Molecules, 2017,22).

FIG. 40 provides non-limiting examples of GSPT1/ERF3 Targeting Ligands, wherein R represents exemplary points at which the Linker can be attached. For additional examples, see the crystal structure PDB 5LZT, 5LZS, 5LZV, 5LZU, 5LZX, 5LZW, 5LZZ, 5LZY (Shao, S., et al. “Decoding Mammalian Ribosome-mRNA States by Translational GTPase Complexes”, Cell, 2016, 167: 1229-1240.e15).

FIG. 41 provides non-limiting examples of IFZV Targeting Ligands, wherein R represents exemplary points at which the Linker can be attached. For additional examples, see the crystal structure PDB (Iyer, S., et al. “The crystal structure of human placenta growth factor-1 (PlGF-1), an angiogenic protein, at 2.0 A resolution.”, J Biol Chem., 2001, 276: 12153-12161); and the crystal structure PDB IRV6 (Christinger, H. W., et al. “The crystal structure of placental growth factor in complex with domain 2 of vascular endothelial growth factor receptor-1”, J Biol Chem., 2004, 279: 10382-10388).

FIG. 42 provides non-limiting examples of NSD2 Targeting Ligands, wherein R represents exemplary points at which the Linker can be attached. For additional examples, see the crystal structure PDB 6XCG (Zhou, M. Q., “Histone-lysine N-methyltransferase NSD2-PWWP1 with compound UNC6934”, to be published); and the crystal structure PDB 6UE6 (Liu, Y., et al. “PWWP1 domain of NSD2 in complex with MR837”, to be published).

FIG. 43 provides non-limiting examples of TAU Targeting Ligands, wherein R represents exemplary points at which the Linker can be attached. For additional examples, see the crystal structure PDB 6VA2, 6VA3 (Chen, J. L. et al. “Design, Optimization, and Study of Small Molecules That Target Tau Pre-mRNA and Affect Splicing.”, J Am Chem Soc., 2020, 142: 8706-8727).

FIG. 44 provides non-limiting examples of CYP17A1 Targeting Ligands, wherein R represents exemplary points at which the Linker can be attached. For additional examples, see the crystal structure PDB 3RUK, 3SWZ (Devore, N.M. et al. “Structures of cytochrome P450 17A1 with prostate cancer drugs abiraterone and TOK-001”, Nature, 2012, 482: 116-119); and the crystal structure PDB 6CHI, 6CIZ, (Fehl, C., et al. “Structure-Based Design of Inhibitors with Improved Selectivity for Steroidogenic Cytochrome P450 17A1 over Cytochrome P450 21A2”, J Med Chem., 2018, 61: 4946-4960).

FIG. 45 provides non-limiting examples SALL4 Targeting Ligands, wherein R represents exemplary points at which the Linker can be attached. For additional examples, see the crystal structure PDB 7BQU, 7BQV (Furihata, H., et al. “Structural bases of IMiD selectivity that emerges by 5-hydroxythalidomide”, Nat Commun., 2020, 11: 4578-4578); and the crystal structure PDB 6UML (Matyskiela, M.E., et al. “Crystal structure of the SALL4-pomalidomide-cereblon-DDB1 complex”, Nat Struct Mol Biol., 2020, 27: 319-322).

FIG. 46 provides non-limiting examples of FAM38 Targeting Ligands, wherein R represents exemplary points at which the Linker can be attached. For additional examples, see the crystal structure PDB 6KG7 (Wang, L., et al. “Structure and mechanogating of the mammalian tactile channel PIEZO2.”, Nature, 2019, 573: 225-229).

FIG. 47 provides non-limiting examples of CYP20A1 Targeting Ligands, wherein R represents exemplary points at which the Linker can be attached. For additional examples, see Durairaj et al. Biological Chemistry, 2020, 401(3), 361-365.

FIG. 48 provides non-limiting examples of HTT Targeting Ligands, wherein R represents exemplary points at which the Linker can be attached. For additional examples, see the crystal structure PDB 5XI1 (Khan, E., et al. “Myricetin Reduces Toxic Level of CAG Repeats RNA in Huntington's Disease (HD) and Spino Cerebellar Ataxia (SCAs).”, ACS Chem Biol., 2018, 13: 180-188).

FIG. 49 provides non-limiting examples of KRAS Targeting Ligands, wherein R represents exemplary points at which the Linker can be attached. For additional examples, see the crystal structure PDB 6CU6 (Hobbs, G.A., et al. “Atypical KRASG12RMutant Is Impaired in PI3K Signaling and Macropinocytosis in Pancreatic Cancer.”, Cancer Discov., 2020, 10: 104-123); ); the crystal structure PDB 6GJ5, 6GJ6, 6GJ8, 6JG7, (“Drugging an Undruggable Pocket on KRAS” PNAS 2019 116 (32) 15823-15829); and the crystal structure PDB 6BP1 (Lu, J., et al. “KRAS Switch Mutants D33E and A59G Crystallize in the State 1 Conformation.”, Biochemistry, 2018, 57: 324-333).

FIG. 50 provides non-limiting examples of NRF2 (NFE2L2) Targeting Ligands, wherein R represents exemplary points at which the Linker can be attached. For additional 5 examples, see the crystal structure PDB 5CGJ (Winkel, A. F., et al. “Characterization of RA839, a Noncovalent Small Molecule Binder to Keap1 and Selective Activator of Nrf2 Signaling.”, J Biol Chem., 2015, 290: 28446-28455); and 6TYM, 6TYP (Ma, B., et al. “Design, synthesis and identification of novel, orally bioavailable non-covalent Nrf2 activators”, Bioorg Med Chem Lett., 2020, 30: 126852-126852).

FIG. 51 provides non-limiting examples of P300 Targeting Ligands, wherein R represents exemplary points at which the Linker can be attached. For additional examples, see the crystal structure PDB 4PZR, 4PZS, 4PZT (Maksimoska, J., et al. “Structure of the p300 Histone Acetyltransferase Bound to Acetyl-Coenzyme A and Its Analogues”, Biochemistry, 2014, 53: 3415-3422); and the crystal structure PDB 6PGU (Gardberg, A. S., et al. “Make the 15 right measurement: Discovery of an allosteric inhibition site for p300-HAT”, Struct Dyn., 2019, 6: 054702-054702).

FIG. 52 provides non-limiting examples of PIK3CA Targeting Ligands, wherein R represents exemplary points at which the Linker can be attached. For additional examples, see the crystal structure PDB 60AC (Rageot, D., et al. “(S)-4-(Difluoromethyl)-5-(4-(3-methylmorpholino)-6-morpholino-1,3,5-triazin-2-yl)pyridin-2-amine (PQR530), a Potent, Orally Bioavailable, and Brain-Penetrable Dual Inhibitor of Class I PI3K and mTOR Kinase”, J Med Chem., 2019, 62: 6241-6261); and the crystal structure PDB 5SX8, 5SWP (Miller, M.S. et al. “Identification of allosteric binding sites for PI3K alpha oncogenic mutant specific inhibitor design.”, Bioorg Med Chem., 2017, 25: 1481-1486).

FIG. 53 provides non-limiting examples of SARM1 Targeting Ligands, wherein R represents exemplary points at which the Linker can be attached. For additional examples, see the crystal structure PDB 6QWV (Sporny, M., et al. “Structural Evidence for an Octameric Ring Arrangement of SARMI”, J Mol Biol., 2019, 431: 3591-3605); and the crystal structure PDB 600Q, 600R, 600T, 600V, 600W (Horsefield, S., et al. “NAD+cleavage activity by animal and plant TIR domains in cell death pathways”, Science, 2019, 365: 793-799).

FIG. 54 provides non-limiting examples of SNCA Targeting Ligands, wherein R represents exemplary points at which the Linker can be attached. For additional examples, see the crystal structure PDB 4I5M, 4I5P, 4I6B, 4I6F, 4I6H (Aubele, D. L., et al. “Selective and brain-permeable polo-like kinase-2 (Plk-2) inhibitors that reduce alpha-synuclein phosphorylation in rat brain”, Chem Med Chem., 2013, 8: 1295-1313).

FIG. 55 provides non-limiting examples of MAPT Targeting Ligands, wherein R represents exemplary points at which the Linker can be attached. For example, the crystal structure PDB 6VI3, 6VHL (Arakhamia, T., et al. “Posttranslational Modifications Mediate the Structural Diversity of Tauopathy Strains”, Cell, 2020, 180: 633-644.e12); and the crystal structure PDB 6FAU, 6FAV, 6FAW, 6FBW, 6FBY, 6F1I4, 6F1I5 (Andrei, S. A., et al. “Inhibition of 14-3-3/Tau by Hybrid Small-Molecule Peptides Operating via Two Different Binding Modes.”, ACS Chem Neurosci., 2018, 9: 2639-2654).

FIG. 56 provides non-limiting examples of PTPN2 or TCPTP Targeting Ligands, wherein R represents exemplary points at which the Linker can be attached. For example, the crystal structure PDB 2FJN, 2FJM (Asante-Appiah, E., et al. “Conformation-assisted inhibition of protein-tyrosine phosphatase-1B elicits inhibitor selectivity over T-cell protein-tyrosine phosphatase”, J Biol Chem., 2006, 281: 8010-8015).

FIG. 57 provides non-limiting examples of STAT3 Targeting Ligands, wherein R represents exemplary points at which the Linker can be attached. The examples shown here derive from compounds in Zheng, W. et al. “MMPP Attenuates Non-Small Cell Lung Cancer Growth by Inhibiting the STAT3 DNA-Binding Activity via Direct Binding to the STAT3 DNA-Binding Domain”, Theranostics 2017, 7(18):4632 and US2006/0247318. For additional examples, see Yang, L. et al. “Novel Activators and Small-Molecule Inhibitors of STAT3 in Cancer”, Cytokine & Growth Factor Reviews 2019, 49, 10-22.

FIG. 58 provides non-limiting examples of MyD88 Targeting Ligands, wherein R represents exemplary points at which the Linker can be attached. The examples shown here derive from compounds in Sucking, C. et al. “Small Molecule Analogues of the parasitic worm product ES-62 interact with the TIR domain of MyD88 to inhibit pro-inflammatory signaling” (2018) Sci. Rep. 8(1):2123 and Loiarro, M. et al. “Pivotal Advance: Inhibition of MyD88 dimerization and recruitment of IRAK1 and IRAK4 by a novel peptidomimetic compound”, Journal of Leukocyte Biology, (2007) 82: 801-810.

FIG. 59 provides non-limiting examples of PTP4A3 Targeting Ligands, wherein R represents exemplary points at which the Linker can be attached. The examples shown here derive from compounds in Ahn, J. et al. “Synthesis and Biological Evaluation of RhodanineD derivatives as PRL-3 Inhibitors”. Bioorganic & Medicinal Chemistry Letters (2006) 16(11):2996-2999 and Min, G. et al. “Rhodanine-Based PRL-3 Inhibitors Blocked the Migration and Invasion of Metastatic Cancer Cells”, Bioorganic & Medicinal Chemistry Letters (2013) 23(13):3769-3774. For additional examples, see Tasker, N. et al. “Tapping the Therapeutic Potential of Protein Tyrosine Phosphatase 4A with Small Molecule Inhibitors” Bioorganic & Medicinal Chemistry Letters (2019) 29(16):2008-2015.

FIG. 60 provides non-limiting examples of SF3B1 Targeting Ligands, wherein R represents exemplary points at which the Linker can be attached. The examples shown here derive from compounds in Kaida, D. et al Spliceostatin A Targets SF3b and Inhibits Both Splicing and Nuclear Retention ofpre-mRNA Nature Chemical Biology (2007) 3:576-583 and Kotake, Y. et al Splicing Factor SF3b as a Target of the Antitumor Natural Product Pladienolide Nature Chemical Biology (2007) 3:570-575. For additional examples, see Effenberger, K. et al. “Modulating Splicing with Small Molecular Inhibitors of the Spliceosome”, WIREs RNA (2016) 8:e1381.

FIG. 61 provides non-limiting examples of ARID1B and ARID2 Targeting Ligands, wherein R represents exemplary points at which the Linker can be attached. For additional examples, see Chory et al. ACS Chemical Biology 2020, 15(6), 1685.

FIG. 62 provides non-limiting examples of Class II BRAF Mutant Targeting Ligands, wherein R represents exemplary points at which the Linker can be attached. For additional examples, see Cho et al. Biochemical andBiophysicalResearch Communications 2020, 352(2), 315.

FIG. 63 provides non-limiting examples of NRASQ⁶1K Targeting Ligands, wherein R represents exemplary points at which the Linker can be attached. For additional examples, see Song et al. Am J Cancer Res 2017, 7(4), 831 and Johnson et al. Curr Treat Options Oncol. 2015, 16(4), 15.

FIG. 64A-64E provide non-limiting examples of ataxia telangiectasia-mutated (ATM) kinase Targeting Ligands wherein R represents exemplary points at which the Linker is attached. Additional examples are provided in J Med Chem, 2019, 62: 2988-3008.

FIG. 65A-65B provide non-limiting examples of ATR Targeting Ligands wherein R represents exemplary points at which the Linker is attached. Additional examples are provided in Journal of Molecular Biology Volume 429, Issue 11, 2 Jun. 2017, Pages 1684-1704.

FIG. 66A-66C provide non-limiting examples of BPTF Targeting Ligands wherein R represents exemplary points at which the Linker is attached. Additional examples are provided in Organic & Biomolecular Chemistry 2020, 18(27): 5174-5182.

FIG. 67A-67B provide non-limiting examples of DNA-PK Targeting Ligands wherein R represents exemplary points at which the Linker is attached. Additional examples are provided in J. Med. Chem. 2020, 63, 7, 3461-3471.

FIG. 68A-68B provide non-limiting examples of elf4E Targeting Ligands wherein R represents exemplary points at which the Linker is attached. Additional examples are provided in J. Am. Chem. Soc. 2020, 142, 4960-4964.

FIG. 69 provides non-limiting examples of TEAD, for example, TEAD1, TEAD2, TEAD3, and/or TEAD4 Targeting Ligands wherein R represents exemplary points at which the Linker is attached.

FIG. 70 provides non-limiting examples of YAP Targeting Ligands wherein R represents exemplary points at which the Linker is attached.

FIG. 71 provides non-limiting examples of Degron formulas of the present invention.

FIG. 72 provides non-limiting examples of B-cell lymphoma 6 protein (BCL6) Targeting Ligands wherein R represents exemplary points at which the Linker is attached.

Additional examples are provided in J. Bio. Chem. 2021, 297, 2, 100928 and Cancer Lett. 2022, 529, 100-111.

FIG. 73A and FIG. 73B provide non-limiting examples of HDAC-co-repressor of repressor element-1 silencing transcription factor (CoREST) Targeting Ligands or CoREST Complex Targeting Ligands wherein R represents exemplary points at which the Linker can be attached. Additional examples are provided in ACS Chem. Neurosci. 2019, 10, 1729-1743.

FIG. 74A-74M provide non-limiting examples of colony stimulating factor 1 receptor (CSF1R) Targeting Ligands wherein R represents exemplary points at which the Linker can be attached. Additional examples are provided in Expert Opin. on Ther. Pat. 2021, 31, 2, 107-117 and Nature Communications 2019, 10, 3758. Crystal structures related to these Targeting Ligands include PDB code 3krj and PDB code 4r7h.

FIG. 75A and FIG. 75B provide non-limiting examples of diacylglycerol kinase (DGK) Targeting Ligands wherein R represents exemplary points at which the Linker can be attached. Additional examples are provided in Cell Chem. Biol. 2017, 24, 870-880, WO2022/187406, and WO2021/127554.

FIG. 76 provides non-limiting examples of son of sevenless homolog 1 (SOS1) Targeting Ligands wherein R represents exemplary points at which the Linker can be attached. Crystal structures related to these Targeting Ligands include Sovi, 6scm, and 7ukr.

FIG. 77 provides non-limiting examples of tyrosine kinse 2 (TYK2) Targeting Ligands wherein R represents exemplary points at which the Linker can be attached. Additional examples are provided in J. Med. Chem. 2023, 66, 4378-4416.

FIG. 78 provides non-limiting examples of ubiquitin specific peptidase 1 (USP1) Targeting Ligands wherein R represents exemplary points at which the Linker can be attached. Additional examples are provided in WO2021/163530.

FIG. 79A-79J provide non-limiting examples of hematopoietic progenitor kinase (HPK1) Targeting Ligands wherein R represents exemplary points at which the Linker can be attached. Additional examples are provided in Expert Opin. on Ther. Pat. 2021, 31, 10, 893-910.

FIG. 80 provides additional non-limiting examples of Degron formulas of the present invention.

FIG. 81 provides a crystal structure of A-1 in chimeric bacterial cereblon protein as described in Example 8.

FIG. 82 provides a crystal structure of A-5 in chimeric bacterial cereblon protein as described in Example 8.

### DETAILED DESCRIPTION OF THE INVENTION

A cereblon binding compound (Degron; a degradation inducing moiety) that covalently binds to cereblon through a sulfonyl fluoride or a sulfonimidoyl fluoride is provided. These compounds can be used to treat disorders mediated by cereblon or mediated by a protein which is degraded by cereblon when a Degron described herein binds to cereblon. Alternatively, a Degron described herein can be used as an intermediate to synthesize a heterobifunctional compound for targeted protein degradation (a Degrader). In certain aspects the Degron includes a linking moiety (a Tail) which can react with an appropriately prepared Targeting Ligand or Targeting Ligand precursor to form a Degrader. Degraders are also provided which include a Degron described herein which can be directly attached to a Targeting Ligand or attached to the Targeting Ligand with a Linker.

In certain aspects the compound of the present invention is of Formula:

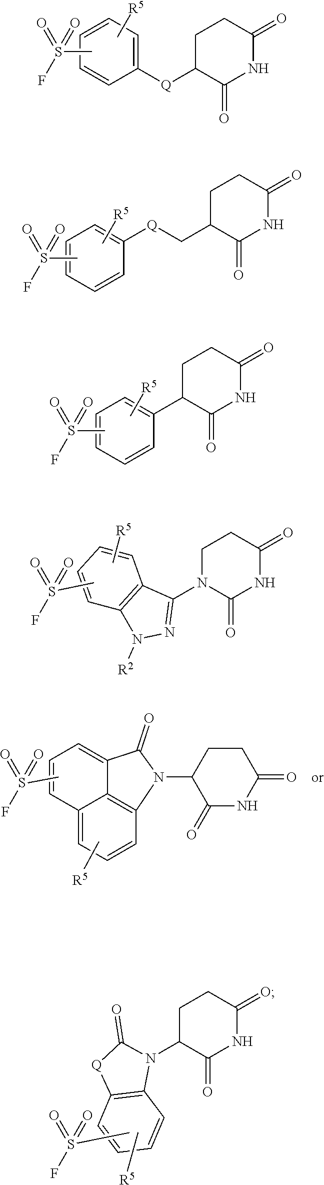

or a pharmaceutically acceptable salt thereof.

In other aspects the compound of the present invention is of Formula:

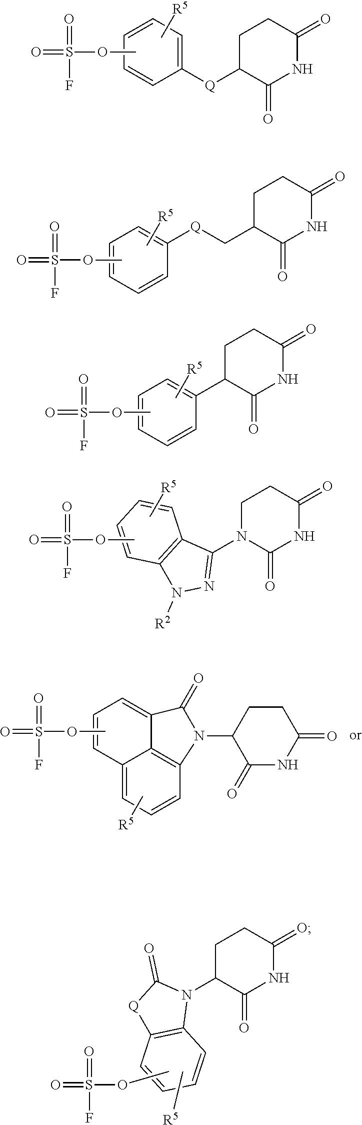

or a pharmaceutically acceptable salt thereof.

### I. DEFINITIONS

Compounds are described using standard nomenclature. Unless defined otherwise, all technical and scientific terms used herein have the same meaning as is commonly understood by one of ordinary skill in the art to which this invention belongs.

The compounds of the present invention may be in the form of a racemate, enantiomer, mixture of enantiomers, diastereomer, mixture of diastereomers, tautomer, N-oxide, isomer, such as rotamer, as if each is specifically described unless specifically excluded by context.

The terms “a” and “an” do not denote a limitation of quantity, but rather denote the presence of at least one of the referenced item(s). The term “or” means “and/or”. Recitation of ranges of values are merely intended to serve as a shorthand method of referring individually to each separate value falling within the range, unless otherwise indicated herein, and each separate value is incorporated into the specification as if it were individually recited herein.

The endpoints of all ranges are included within the range and independently combinable. All methods described herein can be performed in any suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes a compound of the present invention with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁷O, ¹⁸O, ¹⁸F ³¹P, ³²P, ³⁵S, ³⁶Cl, and ¹²⁵I respectively. In certain embodiments, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In certain embodiments, deuterium is 90, 95 or 99% enriched at a desired location.

In certain embodiments, the substitution of a hydrogen atom for a deuterium atom can be provided in any compound of the present invention. In certain embodiments, the substitution of a hydrogen atom for a deuterium atom occurs within one or more groups selected from any of R's or variables described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle, an unsubstituted carbon may be deuterated.

In certain embodiments, a compound of the present invention is isotopically labeled. In certain embodiments, at least one R group independently selected from R¹, R², R⁵, R⁶, R⁷, R¹, R⁹, R¹⁰, R¹¹, R¹³, R¹⁴, R¹⁵, R¹⁶, R¹⁶B, R¹⁷, R^{17A}, R^{17B}, R¹⁸, R^{18A}, R^{18B}, R²⁰, R²¹, R²², R²³, R²⁴, R²⁶, R⁴⁰, and R⁴² is isotopically labeled with 1, 2, or more isotopes as allowed by valence. In certain embodiments, the isotopic label is deuterium. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect). In other embodiments, the isotopic label is ¹³C. In other embodiments, the isotopic label is ¹⁸F.

In certain non-limiting embodiments, the invention includes a solvated form of a compound described herein. The term “solvate” refers to a molecular complex of a compound of the present invention (including a salt thereof) with one or more solvent molecules. Non-limiting examples of solvents are water, ethanol, isopropanol, dimethyl sulfoxide, acetone, and other common organic solvents. The term “hydrate” refers to a molecular complex comprising a compound of the invention and water. Pharmaceutically acceptable solvates in accordance with the invention include those wherein the solvent may be isotopically substituted, e.g., D₂O, acetone-d₆, DMSO-d₆ (dimethyl sulfoxide). A solvate can be in a liquid or solid form.

In certain non-limiting embodiments, the invention includes a prodrug form of a compound described herein. The term “prodrug” refers to a compound which when administered to a host in vivo is converted into a compound of the present invention (the “parent drug”). A prodrug can be used to achieve a desired effect, including for example to improve a chemical or pharmacokinetic property of the parent drug. In certain embodiments the prodrug is an amide, ester, ether, phosphate, phosphonate, sulfonyl, or anhydride versions of a compound of the present invention. The prodrug can be formed by covalently attaching a removable group to the compound of the present invention. Non-limiting examples of reactions to introduce removable groups include acylation, phosphorylation, phosphonylation, phosphoramidation, amidation, esterification, and carbonylation.

A dash (“-”) that is not between two letters or symbols is used to indicate a point of attachment for a substituent. For example, —(C═O)NH₂ is attached through carbon of the carbonyl (C═O) group.

“Alkyl” is a branched or straight chain saturated aliphatic hydrocarbon group. Unless denoted otherwise, “alkyl” is typically a C₁-C₂ alkyl. In certain non-limiting embodiments, the alkyl group contains from 1 to 12 carbon atoms, more generally from 1 to 6 carbon atoms or from 1 to 4 carbon atoms. In certain non-limiting embodiments, the alkyl contains from 1 to 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species and therefore each subset is considered separately disclosed. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentyl, 3-methylpentyl, 2,2-dimethylbutyl, and 2,3-dimethylbutyl. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example, and without limitation, the terms alkyl, alkoxy, haloalkyl, etc., can all be considered to include the cyclic forms of alkyl, unless unambiguously excluded by context.

Non-limiting examples of “cycloalkyl” include dihydro-indene and tetrahydronaphthalene wherein the point of attachment for each group is on the cycloalkyl ring.

For example

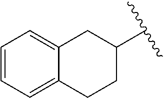 [SMILES: *C1CCc2ccccc2C1]

is an “cycloalkyl” group.

However,

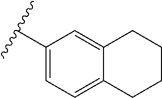 [SMILES: *c1ccc2c(c1)CCCC2]

is an “aryl” group.

The term “alkoxy” denotes a group of the formula —O-alkyl. Examples of alkoxy groups include methoxy, ethoxy, n-propoxy, isopropoxy, n-butoxy, isobutoxy, and tert-butoxy.

The term “cycloalkoxy” denotes a group of the formula —O-cycloalkyl. Examples of cycloalkoxy group include cyclopropoxy, cyclobutoxy, cyclopentyloxy, cyclohexyloxy, cycloheptyloxy, and cyclooctyloxy.

“Alkenyl” is a linear or branched aliphatic hydrocarbon groups having one or more carbon-carbon double bonds that may occur at a stable point along the chain. Unless denoted otherwise, “alkenyl” is typically a C₂-C₈ alkenyl. The specified ranges as used herein indicate an alkenyl group having each member of the range described as an independent species, as described above for the alkyl moiety. In certain non-limiting embodiments, the alkenyl contains from 2 to 12 carbon atoms, from 2 to 6 carbon atoms or from 2 to 4 carbon atoms. In certain embodiments, the alkenyl is C₂, C₂-C₃, C₂-C₄, C₂-C₅, or C₂-C₆alkenyl. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. The term “Alkenyl” also encompasses cycloalkyl or cycloalkyl groups possessing at least one point of unsaturation.

“Alkynyl” is a branched or straight chain aliphatic hydrocarbon group having one or more carbon-carbon triple bonds that may occur at any stable point along the chain. Unless denoted otherwise, “alkynyl” is typically a C₂-C₈ alkynyl. The specified ranges as used herein indicate an alkynyl group having each member of the range described as an independent species, as described above for the alkyl moiety. In certain non-limiting embodiments, the alkynyl contains from 2 to 12 carbon atoms, more generally from 2 to 6 carbon atoms or from 2 to 4 carbon atoms. In certain embodiments, the alkynyl is C₂, C₂-C₃, C₂-C₄, C₂-C₅, or C₂-C₆alkynyl. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. The term “Alkynyl” also encompasses cycloalkyl or cycloalkyl groups possessing at least one triple bond.

“Alkylene” is a bivalent saturated hydrocarbon. Alkylenes, for example, can be a 1, 2, 3, 4, 5, 6, 7 to 8 carbon moiety, 1 to 6-carbon moiety, or an indicated number of carbon atoms, for example C₁-C₂alkylene, C₁-C₃alkylene, C₁-C₄alkylene, C₁-C₅alkylene, or C₁-C₆alkylene.

“Alkenylene” is a bivalent hydrocarbon having at least one carbon-carbon double bond. Alkenylenes, for example, can be a 2 to 8 carbon moiety, 2 to 6-carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, a 2 to 6-carbon moiety, or an indicated number of carbon atoms, for example C₂-C₄alkynylene.

The term “cyano” denotes a —C—N group.

The term “hydroxy” denotes a —OH group.

“Halo” and “Halogen” refers independently to fluorine (F), chlorine (Cl), bromine (Br) or iodine (I). In typical embodiments halogen is fluorine.

“Haloalkyl” is a branched or straight-chain alkyl groups substituted with 1 or more halo atoms described above, up to the maximum allowable number of halogen atoms. Unless denoted otherwise, “haloalkyl” is typically a C₁-C₄ haloalkyl. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include, but are not limited to, trifluoromethyl and pentafluoroethyl.

“Haloalkoxy” indicates a haloalkyl group as described herein attached through an oxygen bridge (oxygen of an alcohol radical).

“Heterocycloalkyl” is an alkyl group as described herein substituted with a heterocyclo group as described herein.

“Arylalkyl” is an alkyl group as described herein substituted with an aryl group as described herein.

Non-limiting examples of “arylalkyl” include;

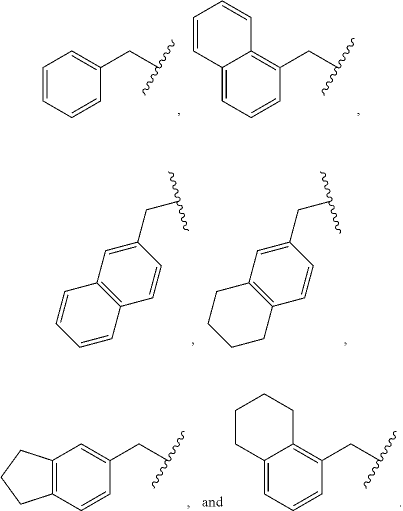

In certain embodiments, “arylalkyl” is

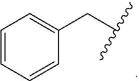 [SMILES: *Cc1ccccc1]

In certain embodiments, the “arylalkyl” refers to a 2-carbon alkyl group substituted with an aryl group.

Non-limiting examples of “arylalkyl” also include:

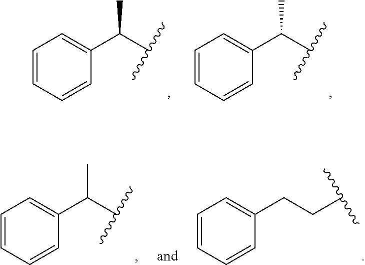

In certain embodiments, the “arylalkyl” refers to a 3-carbon alkyl group substituted with an aryl group.

“Heteroarylalkyl” is an alkyl group as described herein substituted with a heteroaryl group as described herein.

As used herein, “aryl” refers to a radical of a monocyclic or polycyclic (e.g., bicyclic or tricyclic) 4n+2 aromatic ring system (e.g., having 6, 10, or 14 71 electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocycle groups wherein the radical or point of attachment is on the aryl ring, and in such instances, the number of carbon atoms continue to designate the number of carbon atoms in the aryl ring system. The one or more fused carbocyclyl or heterocycle groups can be 4 to 7 or 5 to 7-membered saturated or partially unsaturated carbocyclyl or heterocycle groups that optionally contain 1, 2, or 3 heteroatoms independently selected from nitrogen, oxygen, phosphorus, sulfur, silicon and boron, to form, for example, a 3,4-methylenedioxyphenyl group. In certain embodiments, aryl groups are pendant. An example of a pendant ring is a phenyl group substituted with a phenyl group.

In certain embodiments, “aryl” is a 6-carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

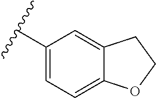 [SMILES: *c1ccc2c(c1)CCO2]

is an “aryl” group.

However,

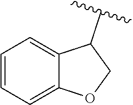 [SMILES: *C1COc2ccccc21]

is a “heterocycle” group.

In certain embodiments, “aryl” is a 6-carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

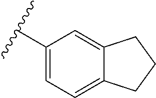 [SMILES: *c1ccc2c(c1)CCC2]

is an “aryl” group.

However,

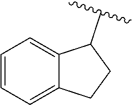 [SMILES: *C1CCc2ccccc21]

is a “cycloalkyl” group.

The term “heterocyclyl”, “heterocycle”, and “heterocyclo” includes saturated, and partially saturated heteroatom-containing ring radicals, where the heteroatoms may be selected from nitrogen, sulfur and oxygen. This term should not be confused with the capitalized term “Heterocyclic Moiety” that is in the present invention and separately defined. Heterocyclic rings comprise monocyclic 3, 4, 5, 6, 7, 8, 9, or 10 membered rings, as well as 5, 6, 7, 8, 9, 10, 11, 12, 13, 14, 15, or 16 membered bicyclic ring systems (which can include bridged fused and spiro-fused bicyclic ring systems). It does not include rings containing —O—O—, -O-S- or -S-S-portions. Examples of saturated heterocyclo groups include saturated 3, 4, 5, or 6-membered heteromonocyclic groups containing 1, 2, 3, or 4 nitrogen atoms [e.g., pyrrolidinyl, imidazolidinyl, piperidinyl, pyrrolinyl, piperazinyl]; saturated 3, 4, 5, or 6-membered heteromonocyclic group containing 1 or 2 oxygen atoms and 1, 2, or 3 nitrogen atoms [e.g., morpholinyl]; saturated 3, 4, 5, or 6-membered heteromonocyclic group containing 1 or 2 sulfur atoms and 1, 2, or 3 nitrogen atoms [e.g., thiazolidinyl]. Examples of partially saturated heterocycle radicals include, but are not limited to, dihydrothienyl, dihydropyranyl, dihydrofuryl, and dihydrothiazolyl. Examples of partially saturated and saturated heterocyclo groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl, isoquinolin-1(2H)-onyl, benzo[d]oxazol-2(3H)-onyl, 1,3-dihydro-2H-benzo[d]midazol-2-onyl, benzo[d]thiazole-2(3H)-onyl, 1,2-dihydro-3H-pyrazol-3-onyl, 2(1H)-pyridinonyl, 2-piperazinonyl, indolinyl, and dihydrothiazolyl.

The term “heterocyclyl”, “heterocycle”, and “heterocyclo” groups also include moieties where heterocycle radicals are fused/condensed with aryl or heteroaryl radicals: such as unsaturated condensed heterocycle group containing 1, 2, 3, 4, or 5 nitrogen atoms, for example, indoline, isoindoline, unsaturated condensed heterocycle group containing 1 or 2 oxygen atoms and 1, 2, or 3 nitrogen atoms, unsaturated condensed heterocycle group containing 1 or 2 sulfur atoms and 1, 2, or 3 nitrogen atoms, and saturated, partially unsaturated and unsaturated condensed heterocycle group containing 1 or 2 oxygen or sulfur atoms.

Additional non-limiting examples of “heterocycle” include indoline, tetrahydroquinoline, tetrahydroisoquinoline, and dihydrobenzofuran wherein the point of attachment for each group is on the heterocycle ring.

For example,

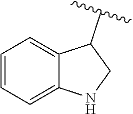 [SMILES: *C1CNc2ccccc21]

is a “heterocycle” group.

However,

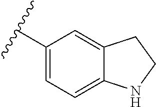 [SMILES: *c1ccc2c(c1)CCN2]

is an “aryl” group.

The term “heteroaryl” denotes a monocyclic or polycyclic (e.g., bicyclic or tricyclic) 4n+2 aromatic ring system (e.g., having 6, 10, or 14 7E electrons shared in a cyclic array) and 1, 2, 3, 4, 5, or 6, heteroatoms independently selected from O, N, and S, wherein the ring nitrogen and sulfur atom(s) are optionally oxidized, and nitrogen atom(s) are optionally quarternized. Examples include, but are not limited to, unsaturated 5- to 6-membered heteromonocyclyl groups containing 1, 2, 3, or 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- or 6-membered heteromonocyclic groups containing an oxygen atom, for example, pyranyl, 2-furyl, 3-furyl, etc.; unsaturated 5- or 6-membered heteromonocyclic groups containing a sulfur atom, for example, 2-thienyl, 3-thienyl, etc.; unsaturated 5- or 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 or 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. Additional examples include 8-, 9-, or 10-membered heteroaryl bicyclic groups such as indazolyl, indolyl, imidazo[1,5-a]pyridinyl, benzimidazolyl, 4(3H)-quinazolinonyl, quinolinyl, isoquinolinyl, isoindolyl, thienothienyl, indolizinyl, benzofuranyl, isobenzofuranyl, benzothienyl, isobenzothienyl, benzoxazolyl, benzothiazolyl, purinyl, coumarinyl, cinnolinyl, and triazolopyridinyl.

The term “bicycle” refers to a ring system wherein two rings are fused together and each ring is independently selected from carbocycle, heterocycle, aryl, and heteroaryl. Bicyclic ring systems also include spiro-fused bicyclic ring systems. Non-limiting examples of bicycle groups include:

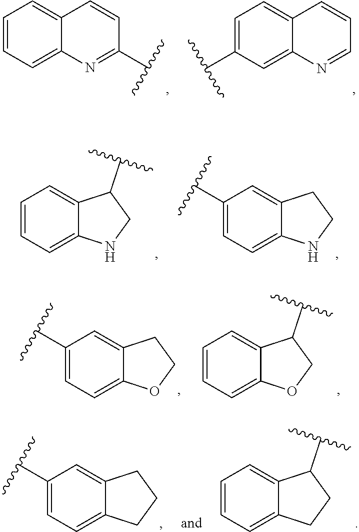

When the term “bicycle” is used in the context of a bivalent residue such as Linker the attachment points can be on separate rings or on the same ring. In certain embodiments, both attachment points are on the same ring. In certain embodiments, both attachment points are on different rings. Non-limiting examples of bivalent bicycle groups include:

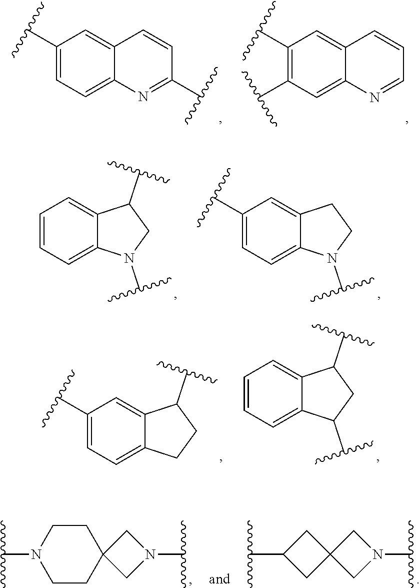

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6 unless excluded by context. This applies regardless of the breadth of the range.

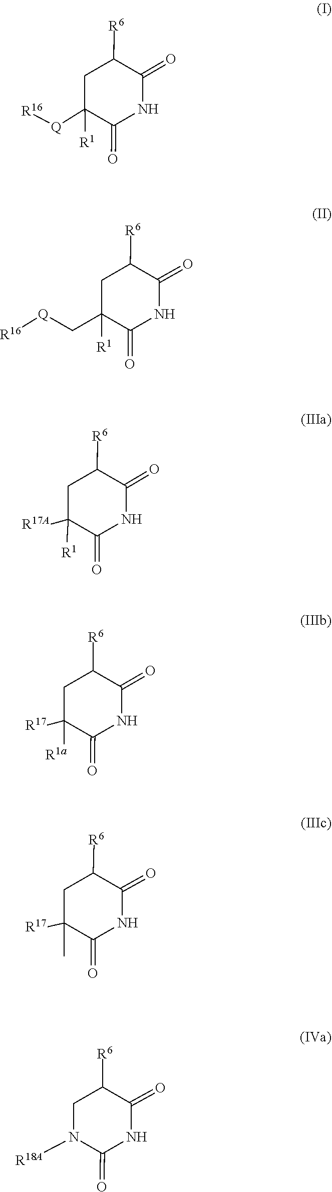

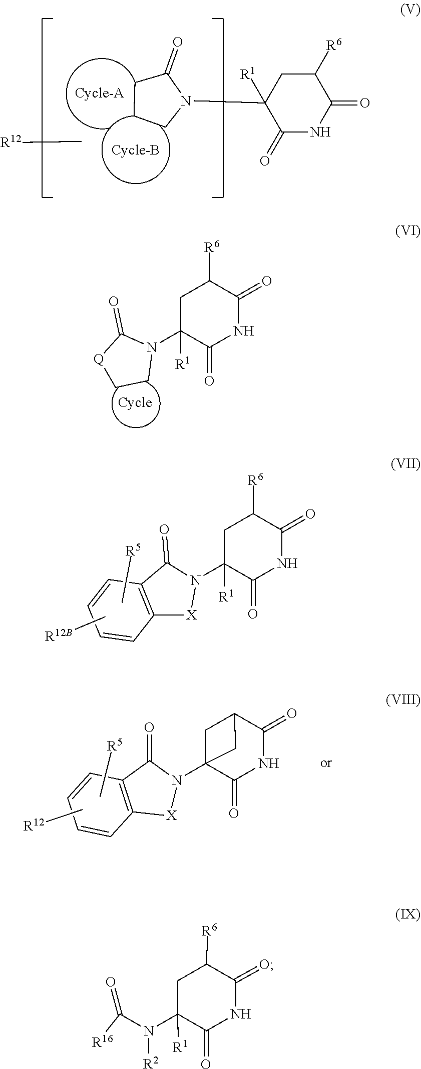

### Embodiments of Formula I

In certain embodiments the compound of the present invention is of Formula:

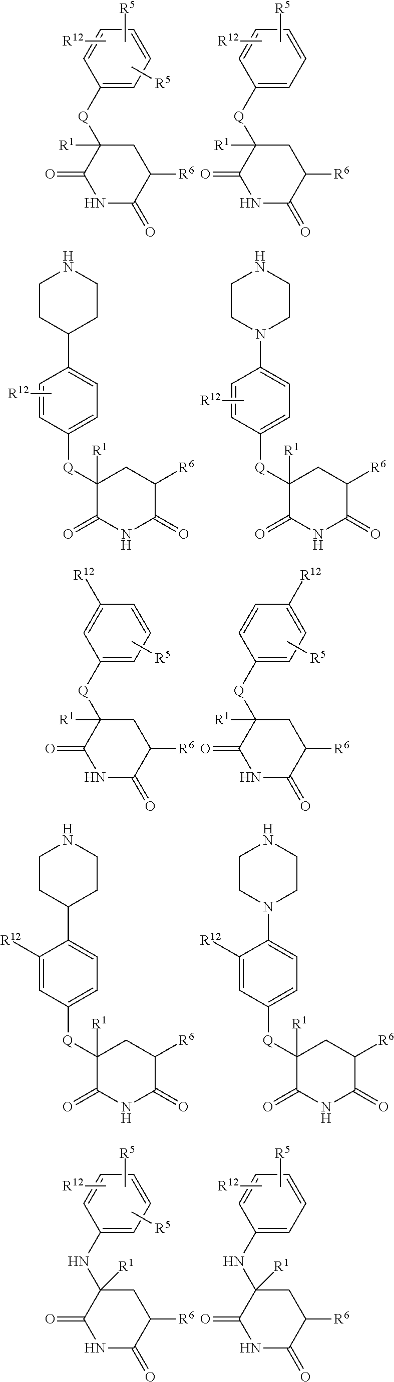

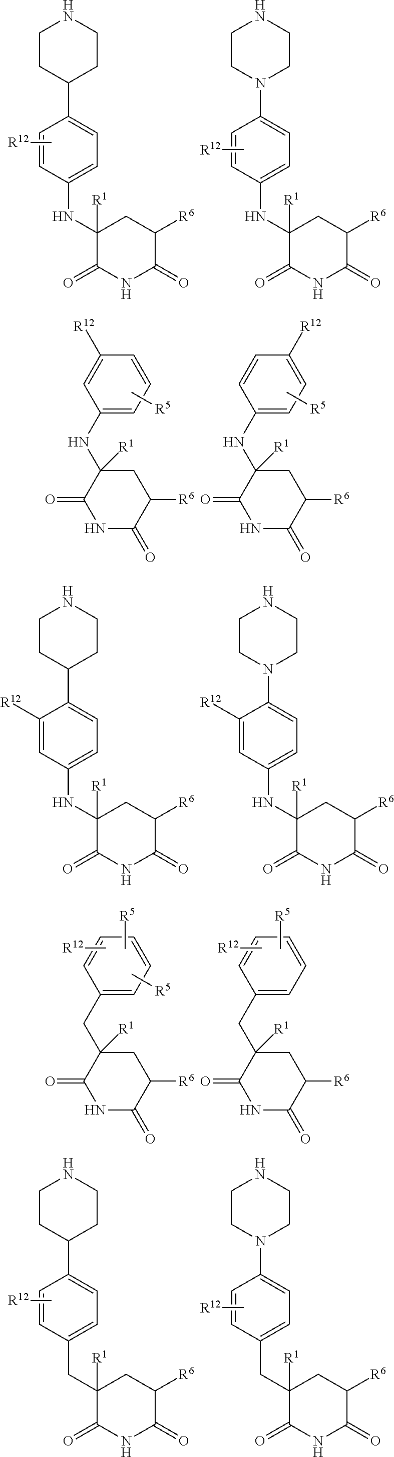

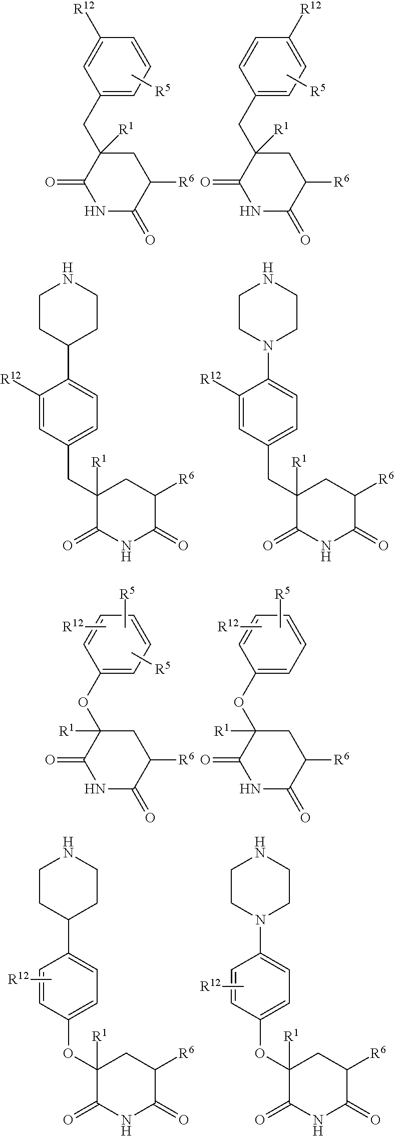

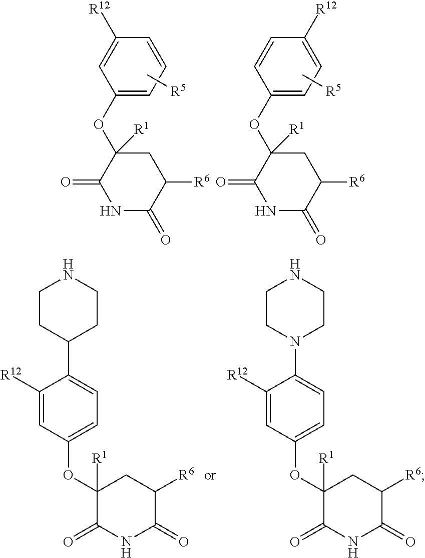

In other embodiments the compound of the present invention is of Formula:

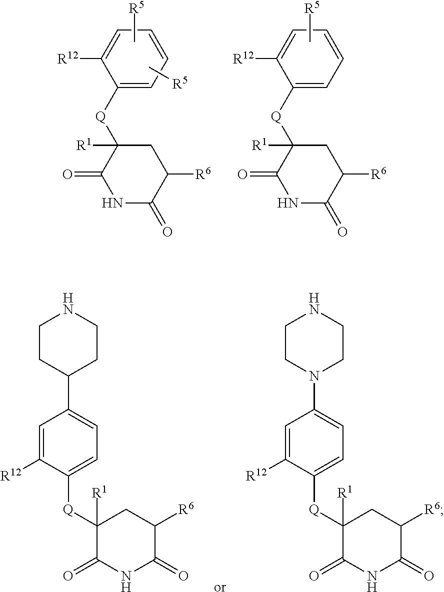

or a pharmaceutically acceptable salt thereof.

In other embodiments the compound of the present invention is of Formula:

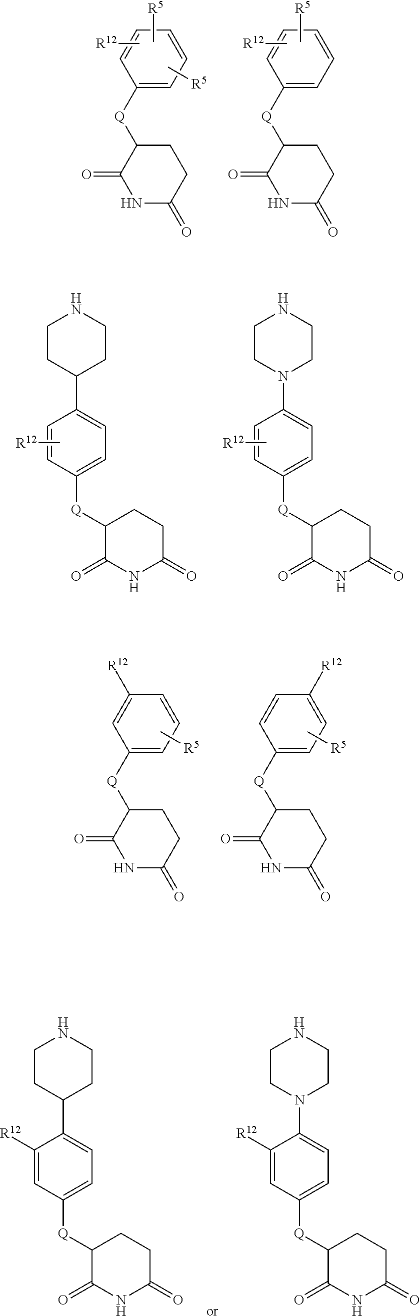

or a pharmaceutically acceptable salt thereof.

In other embodiments the compound of the present invention is of Formula:

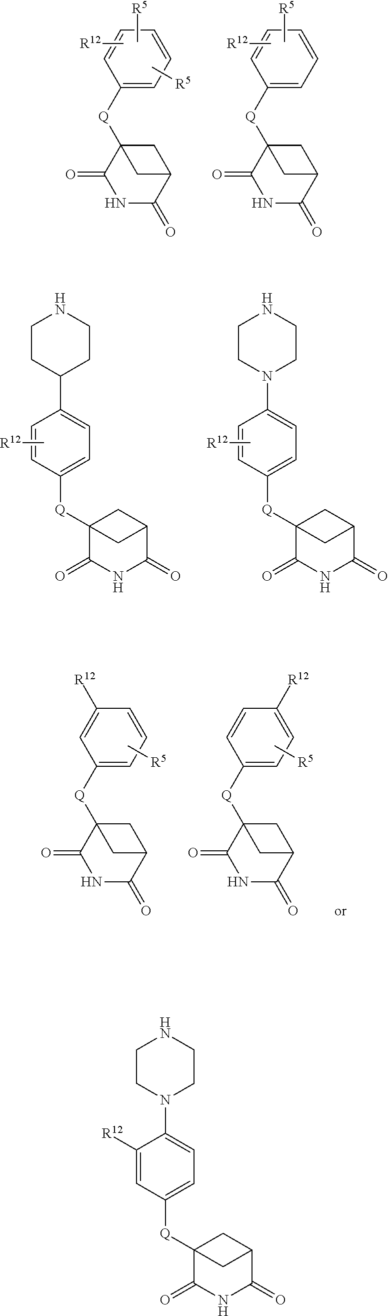

or a pharmaceutically acceptable salt thereof.

Non-limiting examples of compounds of Formula I include:

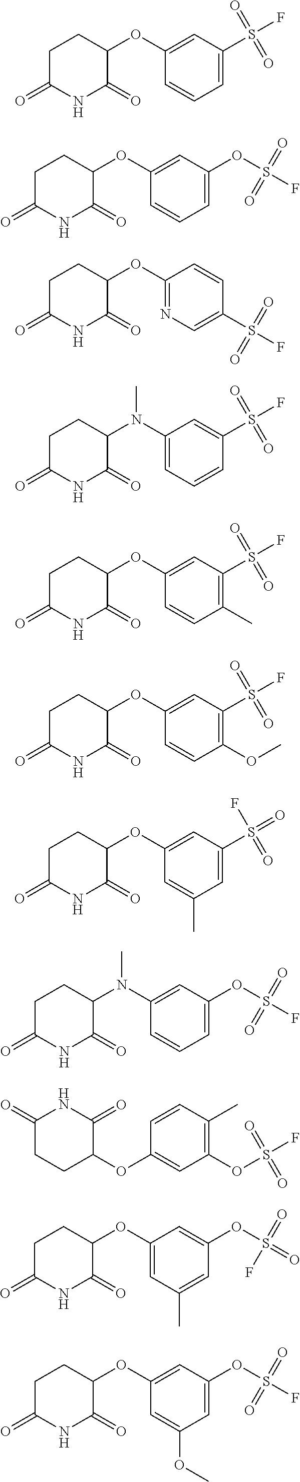

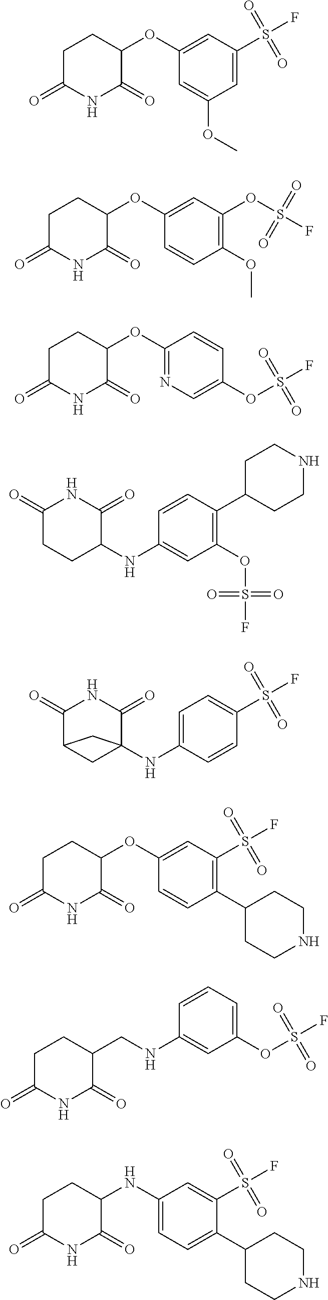

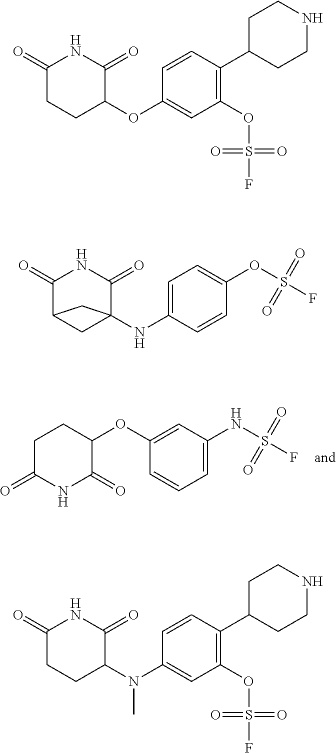 [SMILES: O=C1CCC(Oc2ccc(C3CCNCC3)c(OS(=O)(=O)F)c2)C(=O)N1 ; O=C1NC(=O)C2(Nc3ccc(OS(=O)(=O)F)cc3)CC1C2 ; O=C1CCC(Oc2cccc(NS(=O)(=O)F)c2)C(=O)N1 ; CN(c1ccc(C2CCNCC2)c(OS(=O)(=O)F)c1)C1CCC(=O)NC1=O]

or a pharmaceutically acceptable salt thereof.

In certain embodiments the compound of the present invention is of Formula:

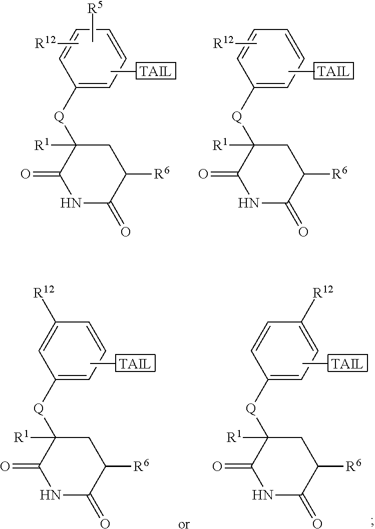

or a pharmaceutically acceptable salt thereof.

In other embodiments the compound of the present invention is of Formula:

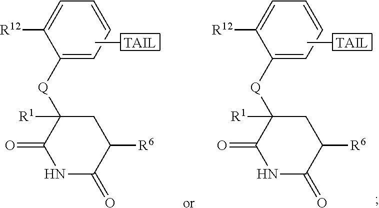

or a pharmaceutically acceptable salt thereof.

In certain embodiments the compound of the present invention is of Formula:

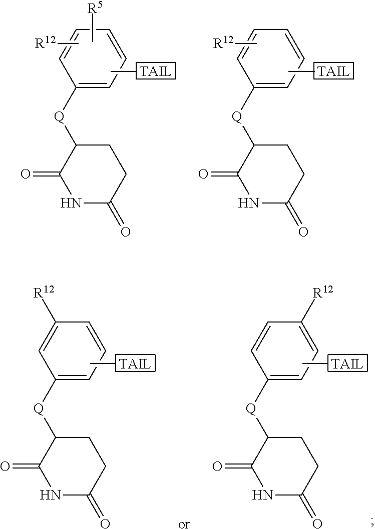

or a pharmaceutically acceptable salt thereof.

In other embodiments the compound of the present invention is of Formula:

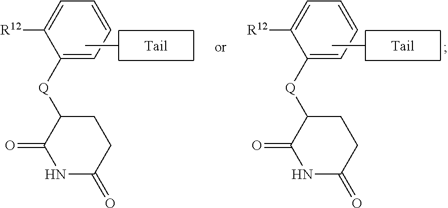

or a pharmaceutically acceptable salt thereof.

In certain embodiments the compound of the present invention is of Formula:

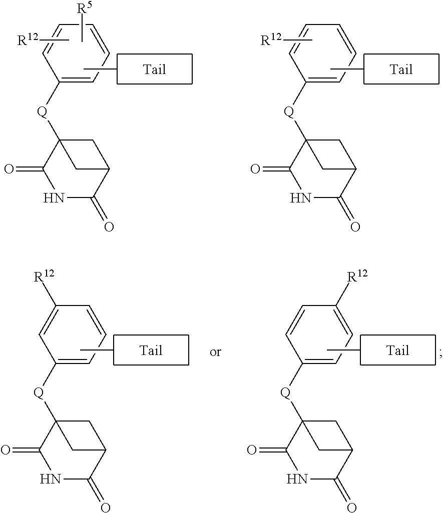

or a pharmaceutically acceptable salt thereof.

In other embodiments the compound of the present invention is of Formula:

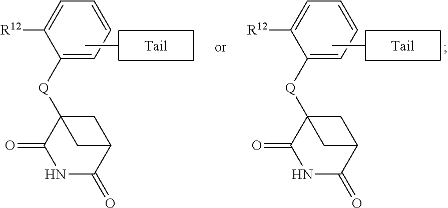

or a pharmaceutically acceptable salt thereof.

In certain embodiments the compound of the present invention is of Formula:

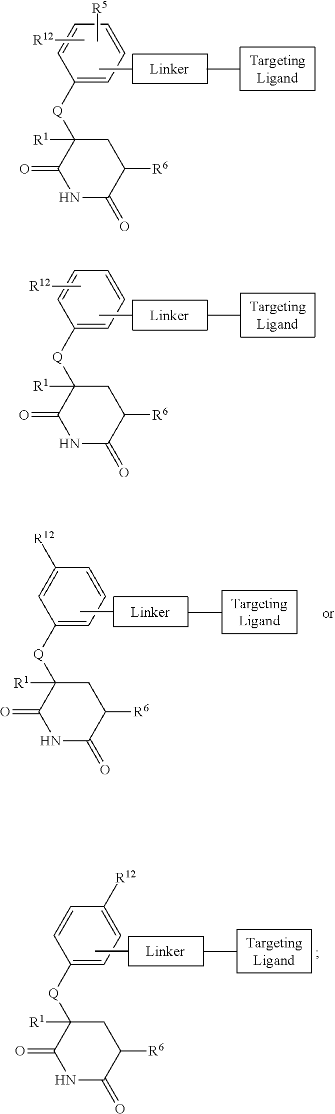

or a pharmaceutically acceptable salt thereof.

In other embodiments the compound of the present invention is of Formula:

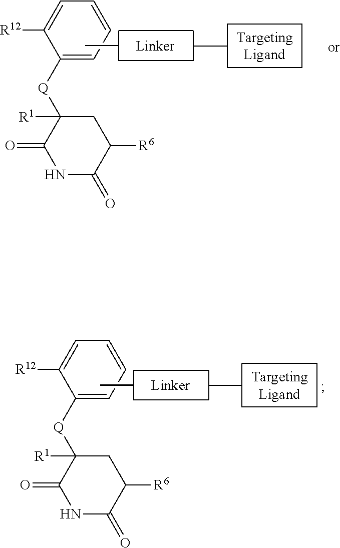

or a pharmaceutically acceptable salt thereof.

In certain embodiments the compound of the present invention is of Formula:

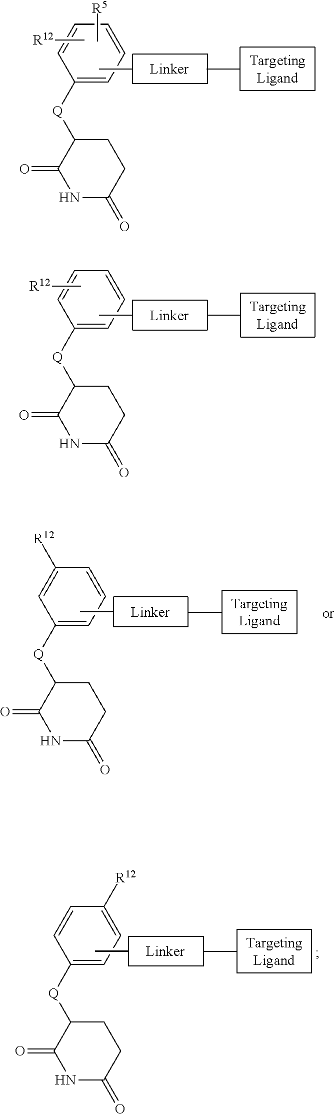

or a pharmaceutically acceptable salt thereof.

In other embodiments the compound of the present invention is of Formula:

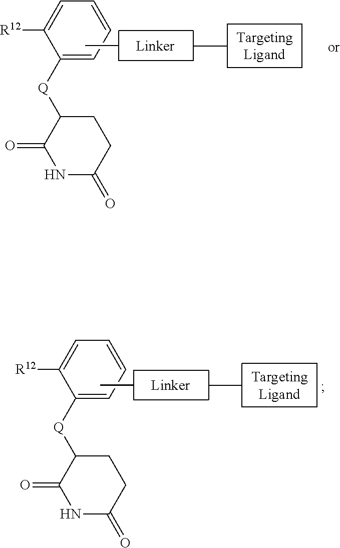

or a pharmaceutically acceptable salt thereof.

In certain embodiments the compound of the present invention is of Formula:

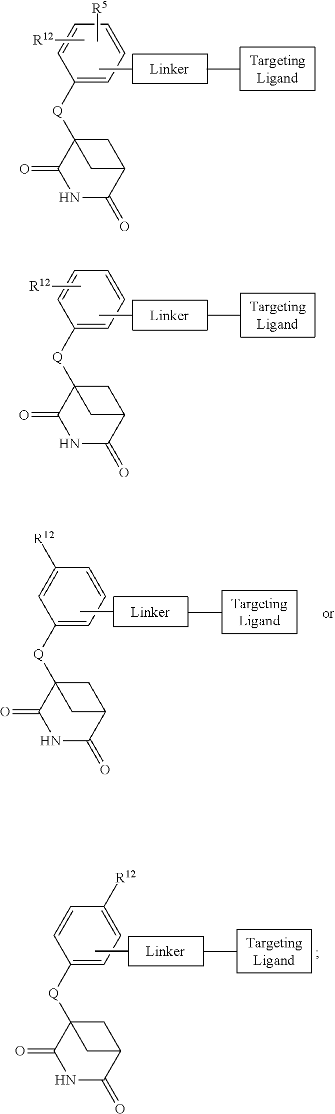

or a pharmaceutically acceptable salt thereof.

In other embodiments the compound of the present invention is of Formula:

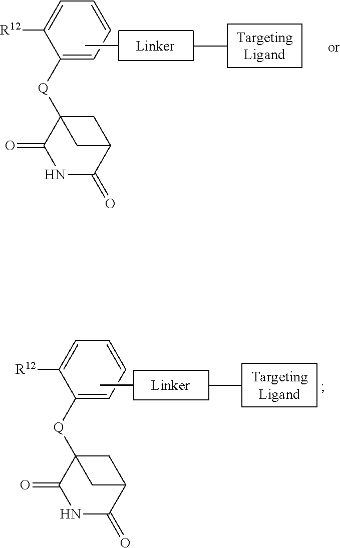

or a pharmaceutically acceptable salt thereof.

Non-limiting examples of compounds of Formula IC include:

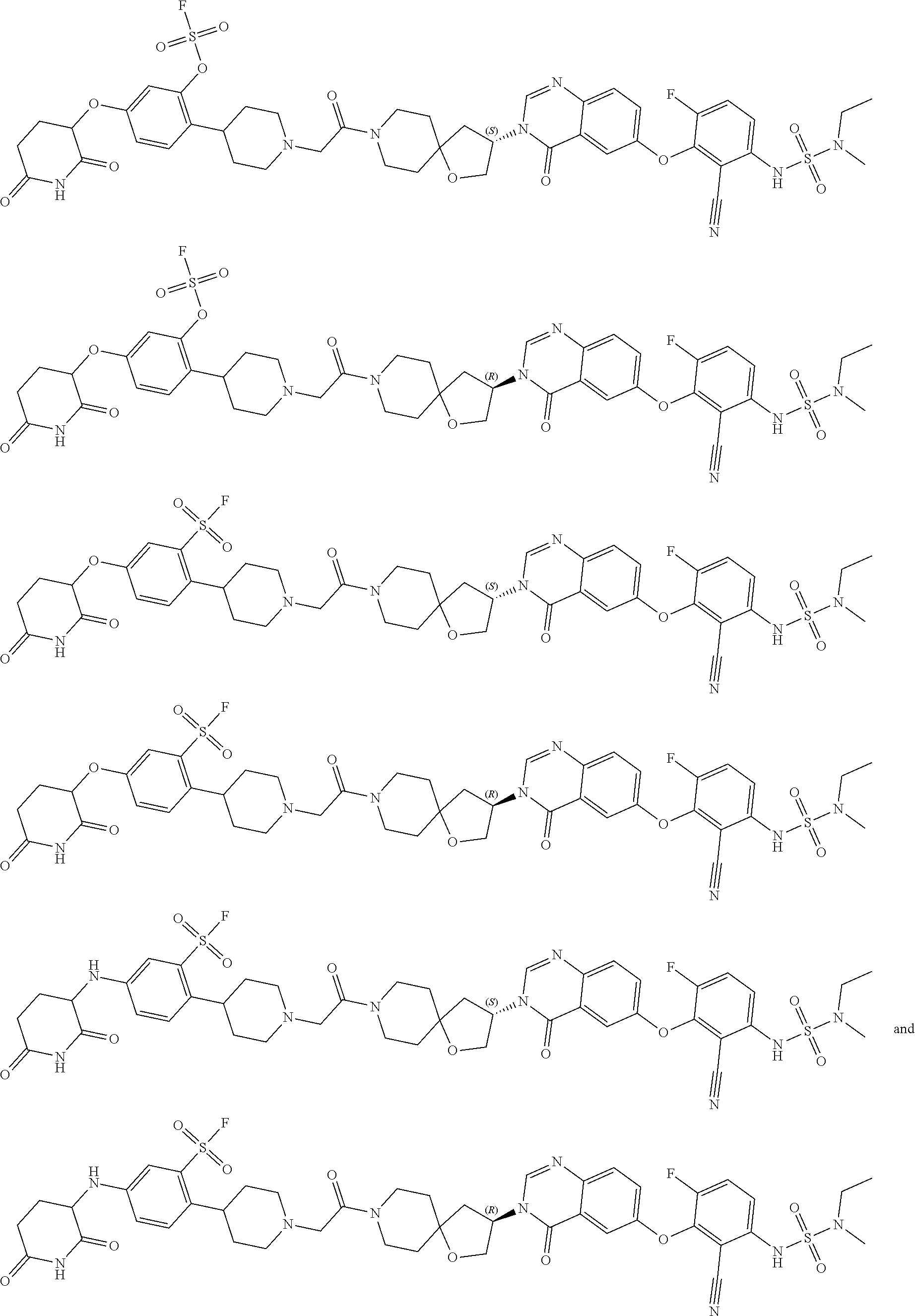

or a pharmaceutically acceptable salt thereof.

Additional non-limiting examples of compounds of the present invention include:

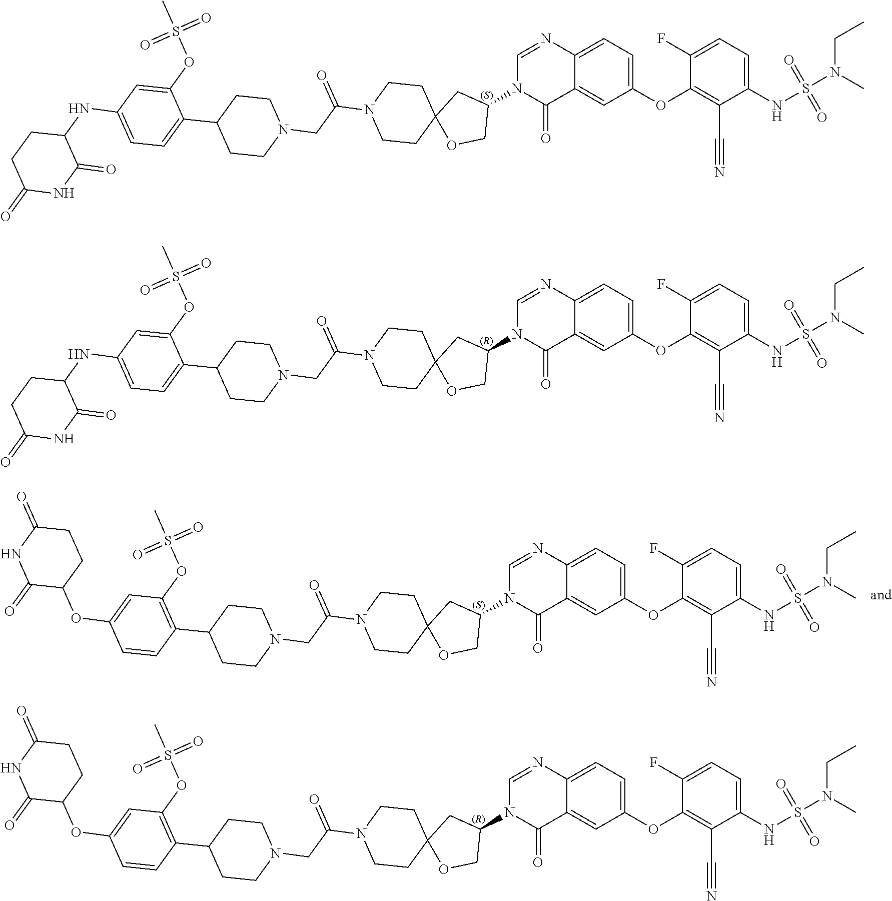

or a pharmaceutically acceptable salt thereof.

In certain embodiments Q is NH.

In certain embodiments Q is NCH₃.

In certain embodiments Q is O.

In certain embodiments Q is S.

In certain embodiments Q is CH₂.

### Embodiments of Formula II

In certain embodiments the compound of the present invention is of Formula:

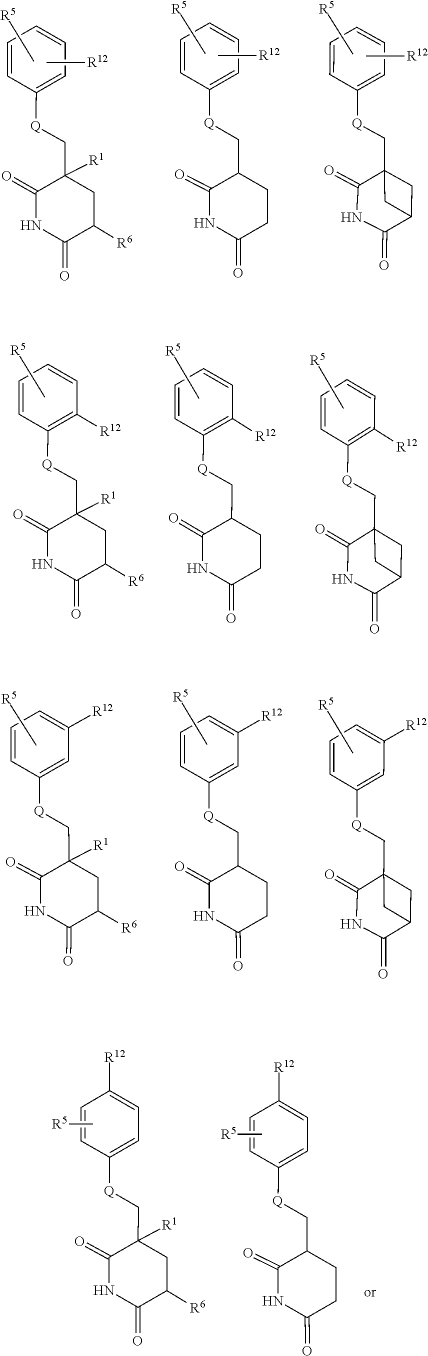

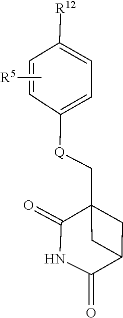

or a pharmaceutically acceptable salt thereof.

Non-limiting examples of compounds of Formula II include:

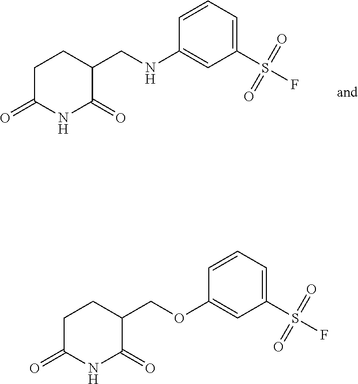 [SMILES: O=C1CCC(CNc2cccc(S(=O)(=O)F)c2)C(=O)N1 ; O=C1CCC(COc2cccc(S(=O)(=O)F)c2)C(=O)N1]

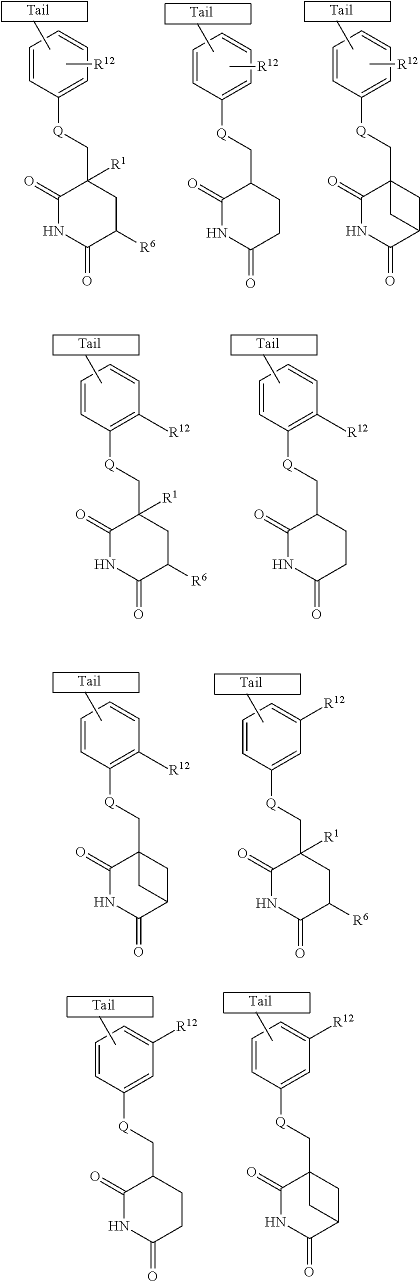

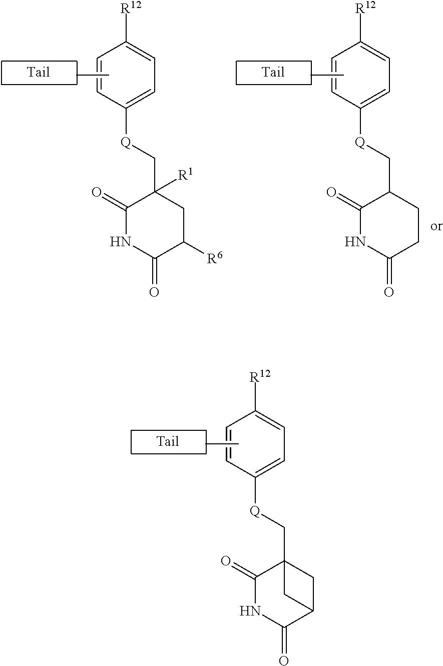

or a pharmaceutically acceptable salt thereof.

In certain embodiments the compound of the present invention is of Formula:

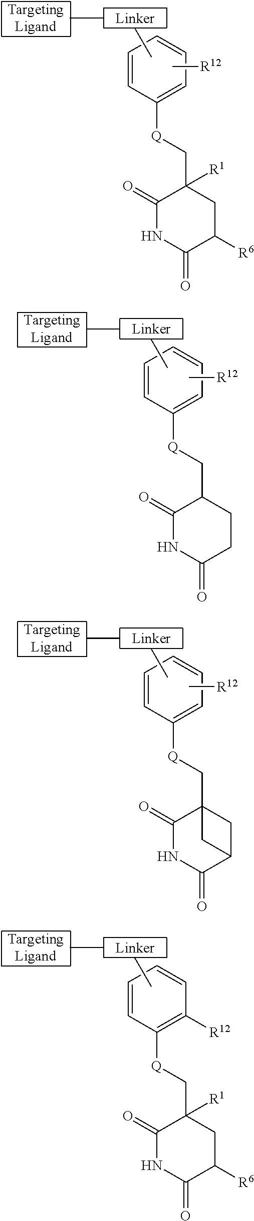

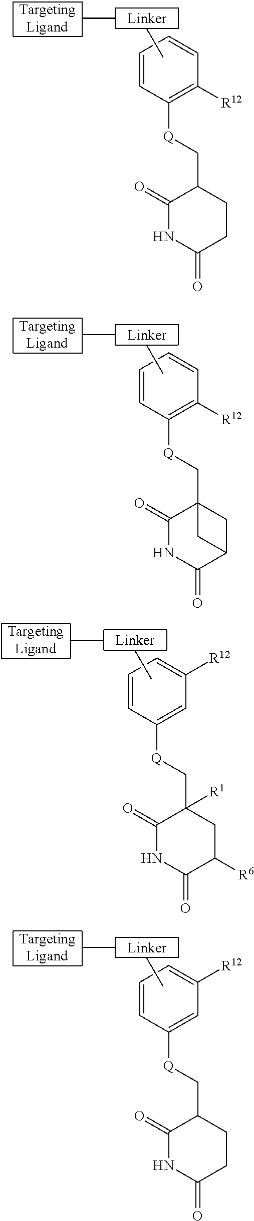

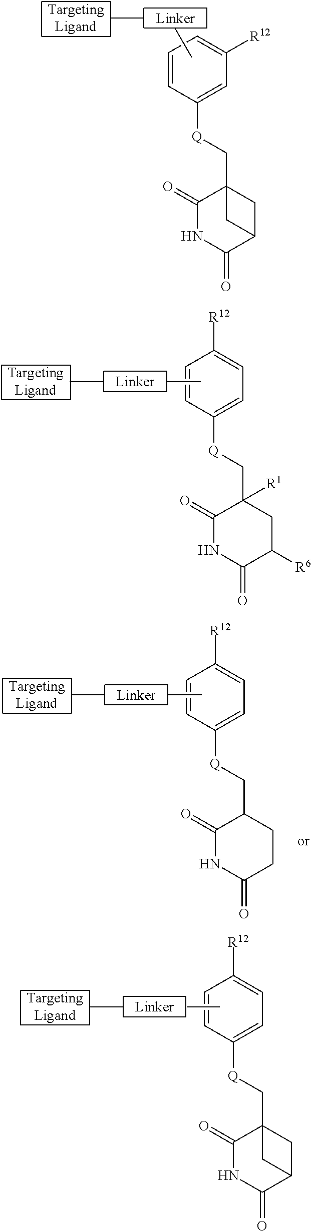

or a pharmaceutically acceptable salt thereof.

In certain embodiments Q is NH.

In certain embodiments Q is NCH₃.

In certain embodiments Q is O.

In certain embodiments Q is S.

In certain embodiments Q is CH₂.

### Embodiments of Formula IIIa, IIIb, and IIIc

In certain embodiments the compound of the present invention is of Formula:

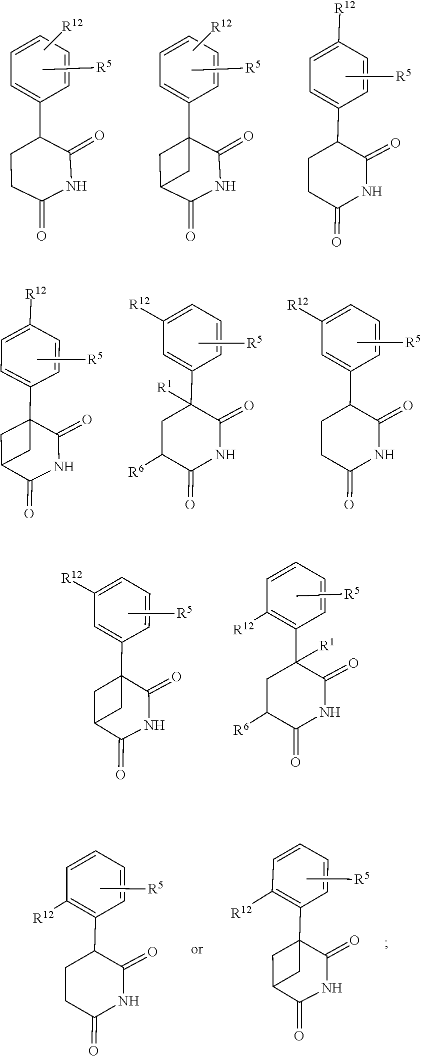

or a pharmaceutically acceptable salt thereof.

In certain embodiments the compound of the present invention is of Formula:

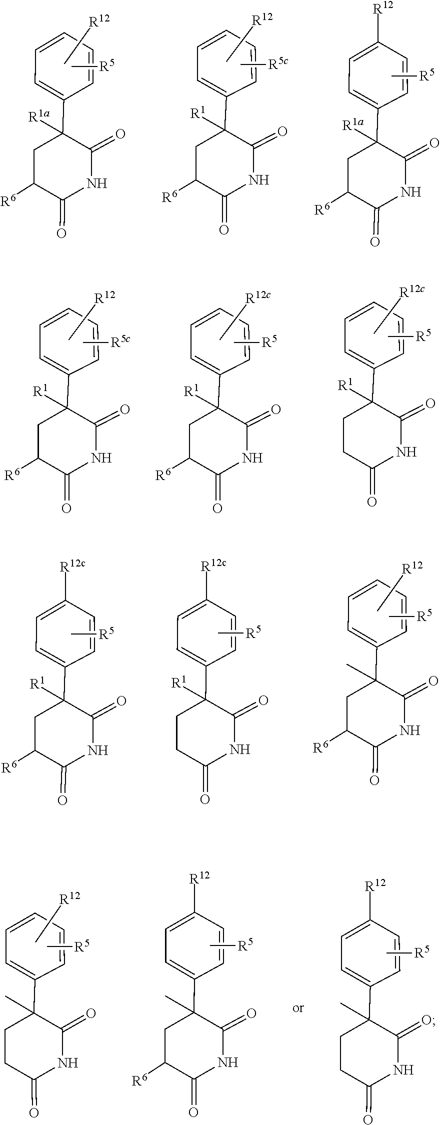

or a pharmaceutically acceptable salt thereof.

In alternative embodiments the compound of the present invention is of Formula:

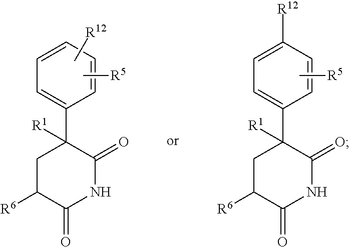

or a pharmaceutically acceptable salt thereof.

In certain embodiments the compound of the present invention is of Formula:

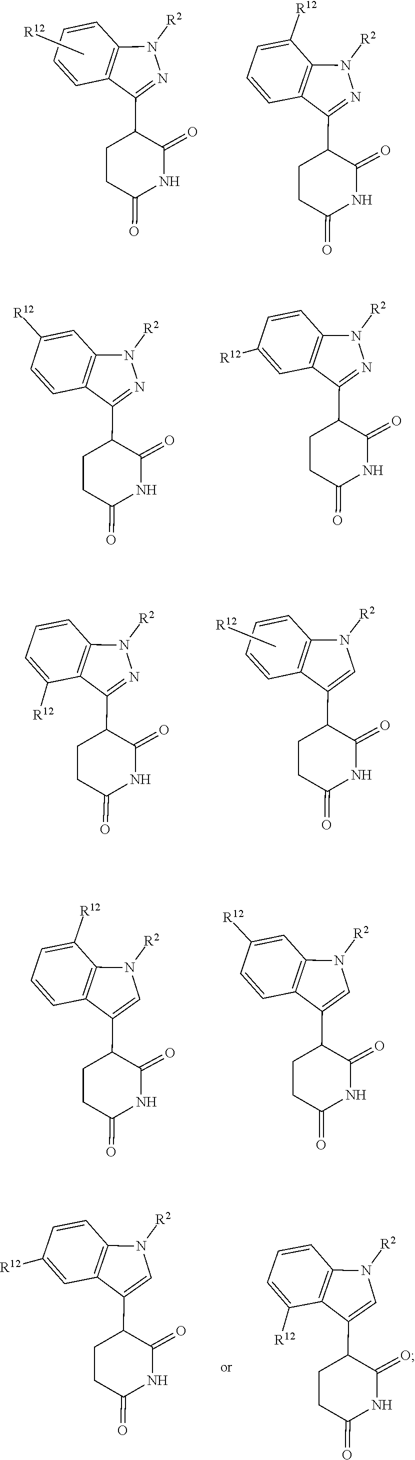

or a pharmaceutically acceptable salt thereof.

In certain embodiments the compound of the present invention is of Formula:

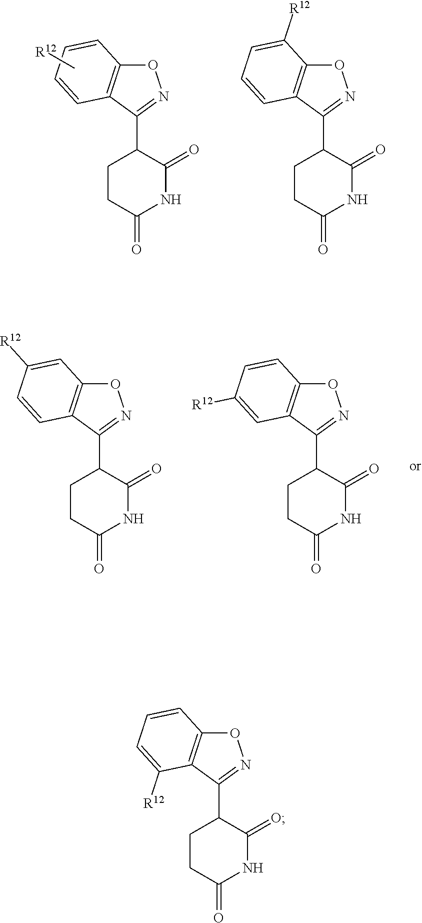

or a pharmaceutically acceptable salt thereof.

In certain embodiments the compound of the present invention is of Formula:

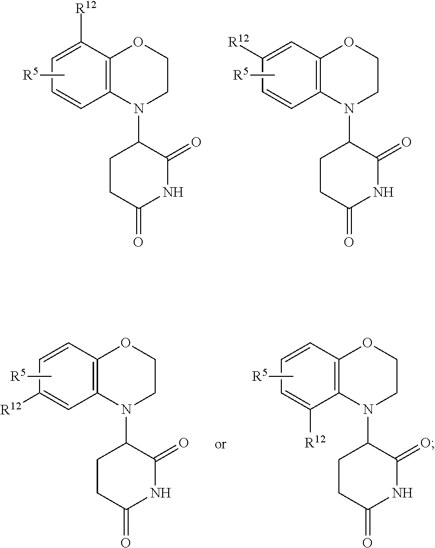

or a pharmaceutically acceptable salt thereof.

Non-limiting examples of compounds of Formula III include:

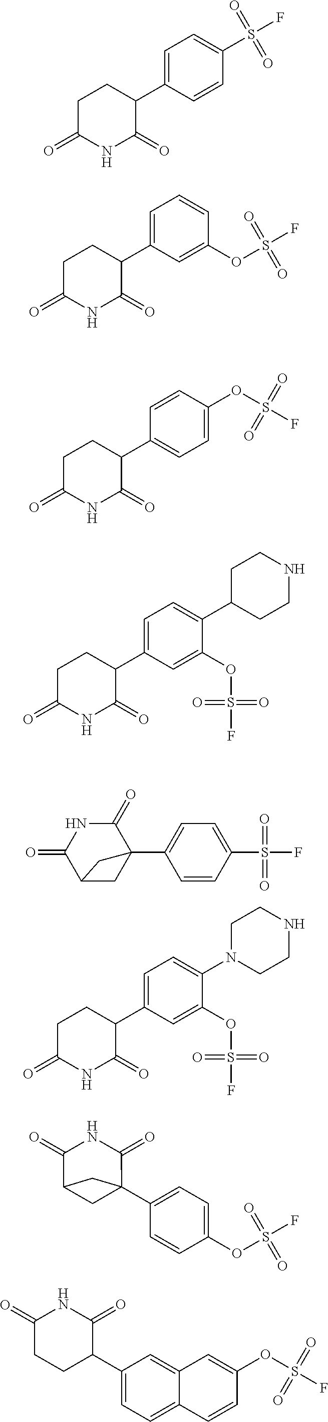

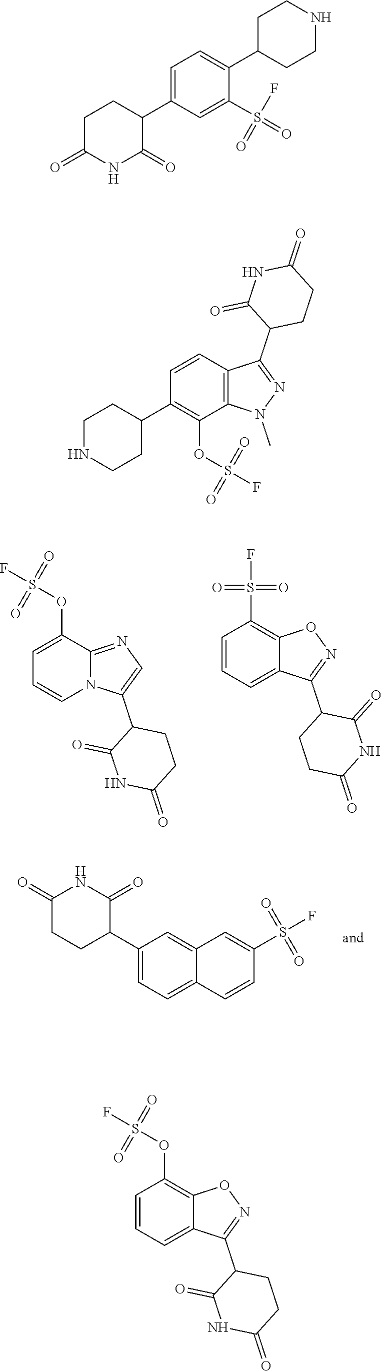 [SMILES: O=C1CCC(c2ccc(C3CCNCC3)c(S(=O)(=O)F)c2)C(=O)N1 ; Cn1nc(C2CCC(=O)NC2=O)c2ccc(C3CCNCC3)c(OS(=O)(=O)F)c21 ; O=C1CCC(c2cnc3c(OS(=O)(=O)F)cccn23)C(=O)N1 ; O=C1CCC(c2noc3c(S(=O)(=O)F)cccc23)C(=O)N1 ; O=C1CCC(c2ccc3ccc(S(=O)(=O)F)cc3c2)C(=O)N1 ; O=C1CCC(c2noc3c(OS(=O)(=O)F)cccc23)C(=O)N1]

or a pharmaceutically acceptable salt thereof.

In certain embodiments the compound of the present invention is of Formula:

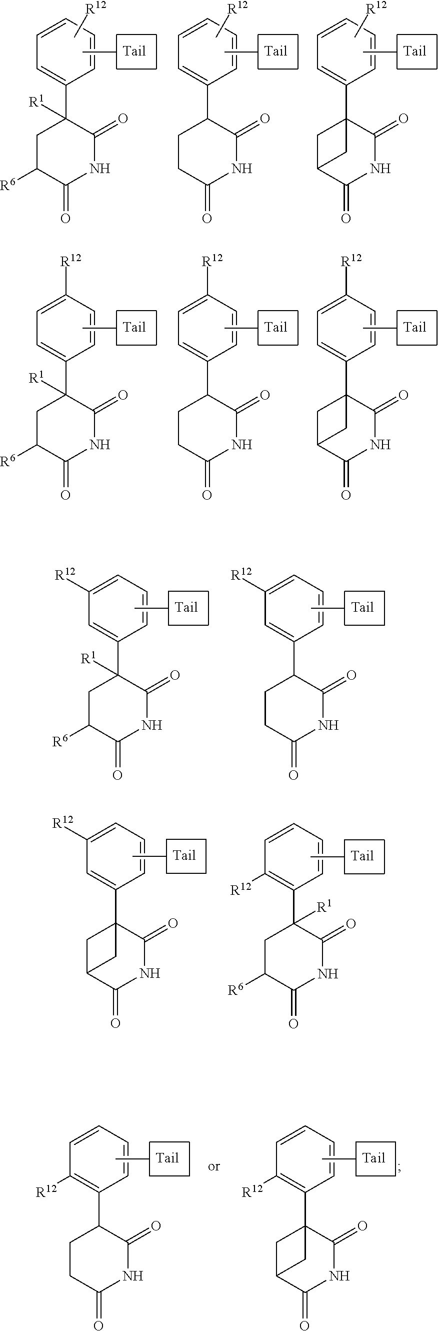

or a pharmaceutically acceptable salt thereof.

In certain embodiments the compound of the present invention is of Formula:

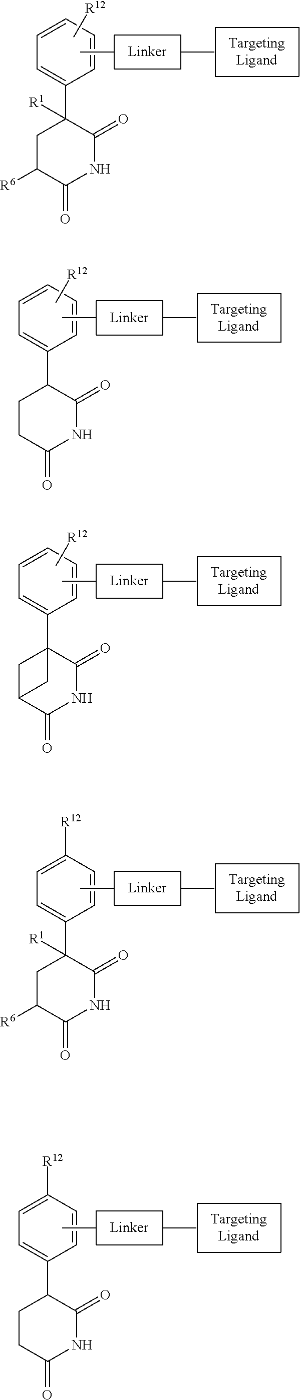

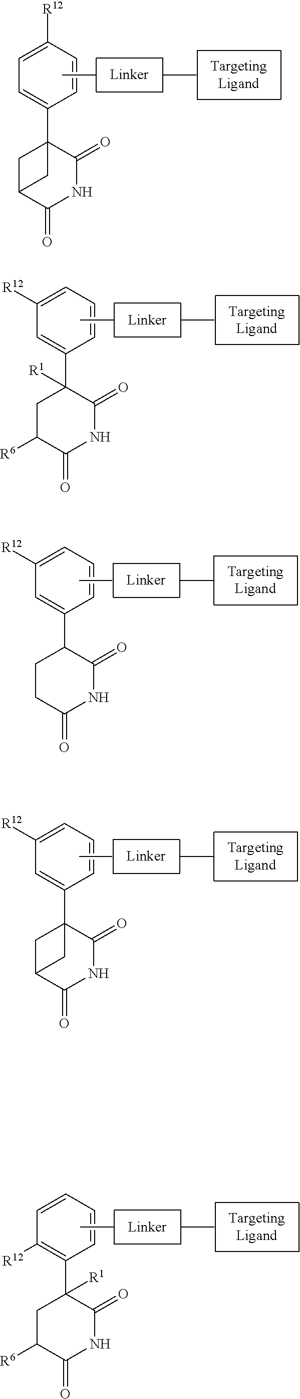

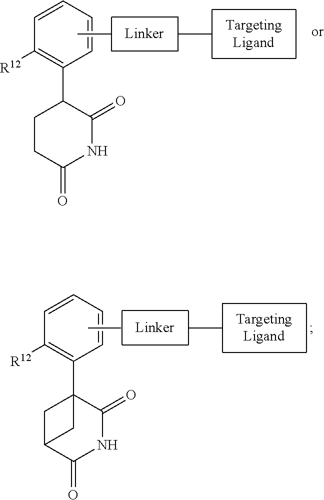

or a pharmaceutically acceptable salt thereof.

Non-limiting examples of compounds of Formula IIIC include:

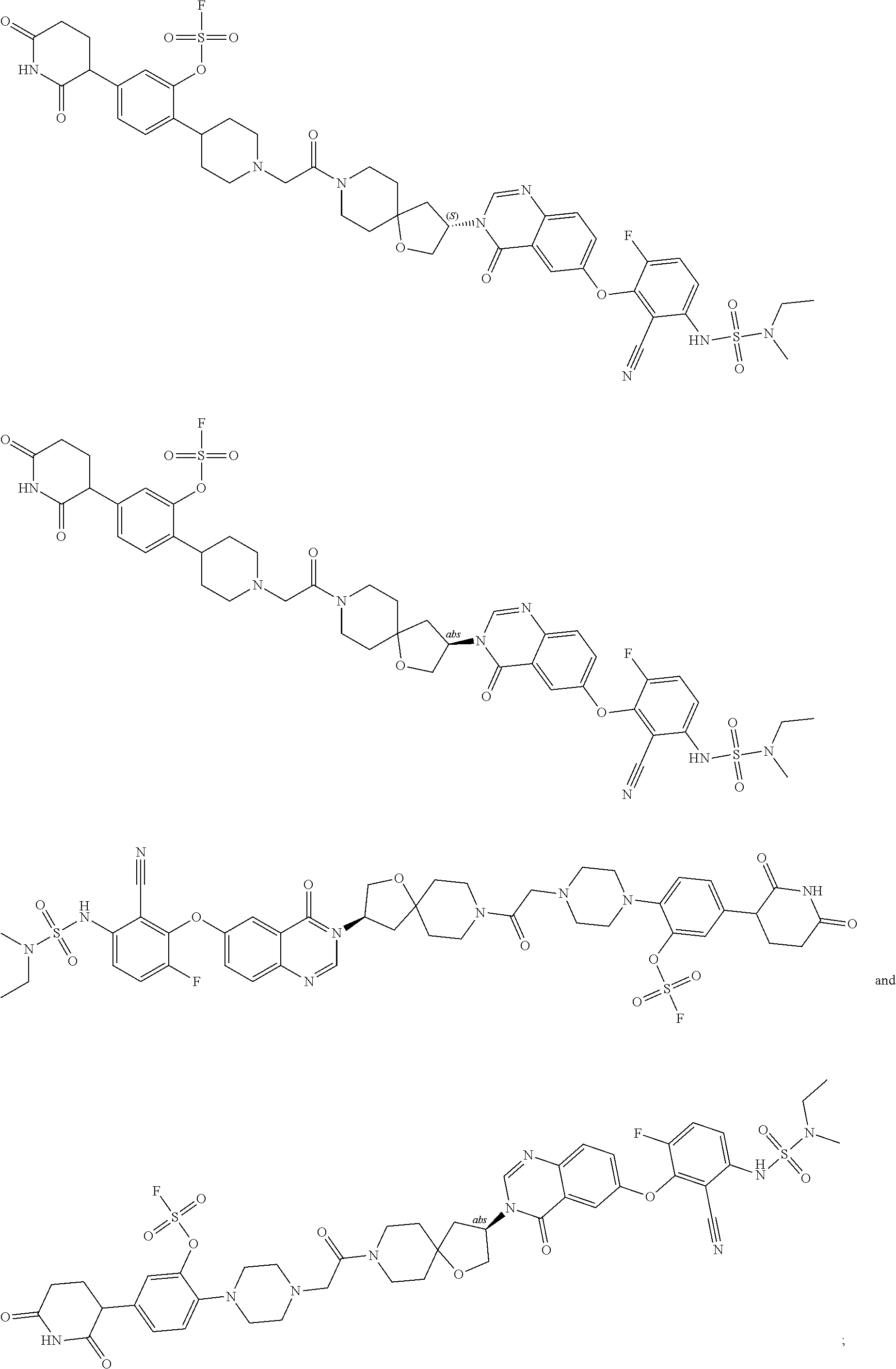

or a pharmaceutically acceptable salt thereof.

Additional non-limiting examples of the compound of the present invention include:

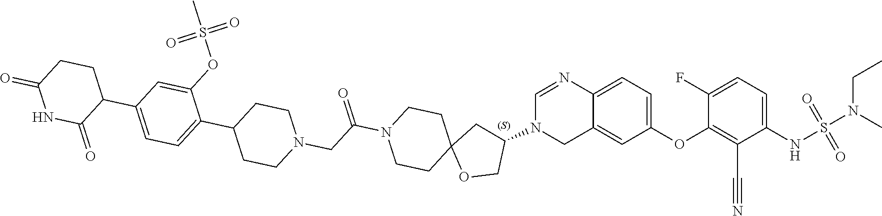

or a pharmaceutically acceptable salt thereof.

### Embodiments of Formula IV

In certain embodiments the compound of the present invention is of Formula:

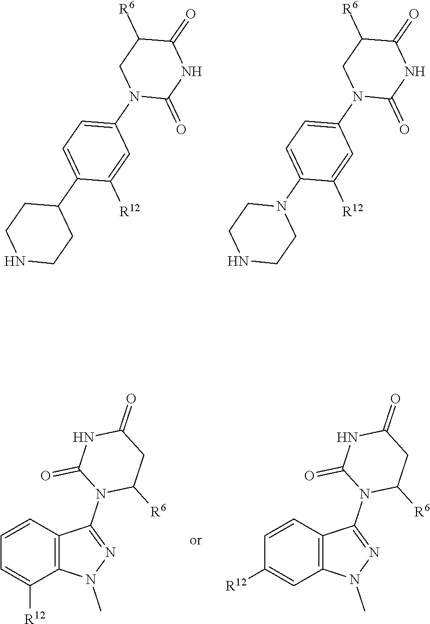

or a pharmaceutically acceptable salt thereof.

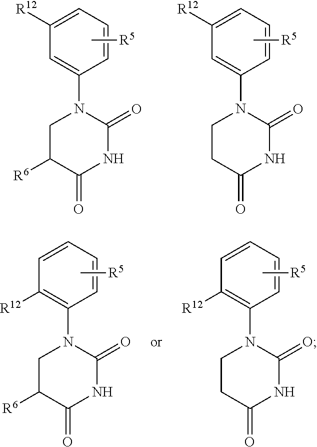

In certain embodiments the compound of the present invention is of Formula:

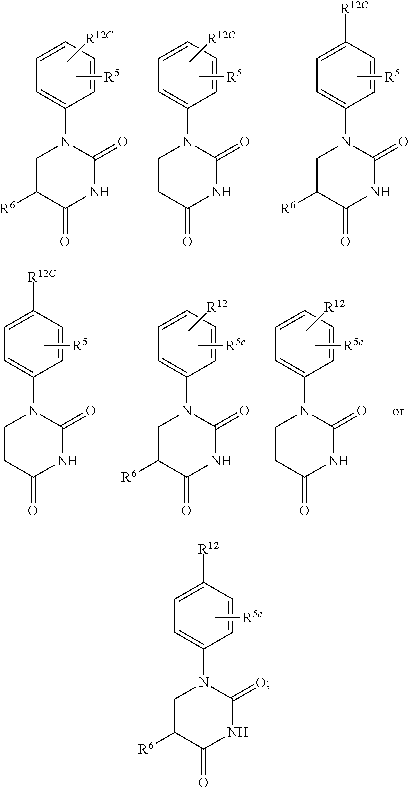

or a pharmaceutically acceptable salt thereof.

In alternative embodiments the compound of the present invention is of Formula:

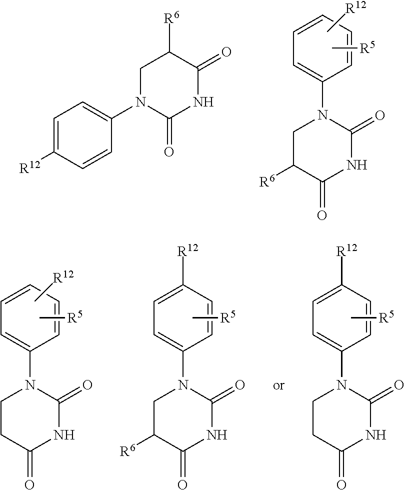

or a pharmaceutically acceptable salt thereof.

In certain embodiments the compound of the present invention is of Formula:

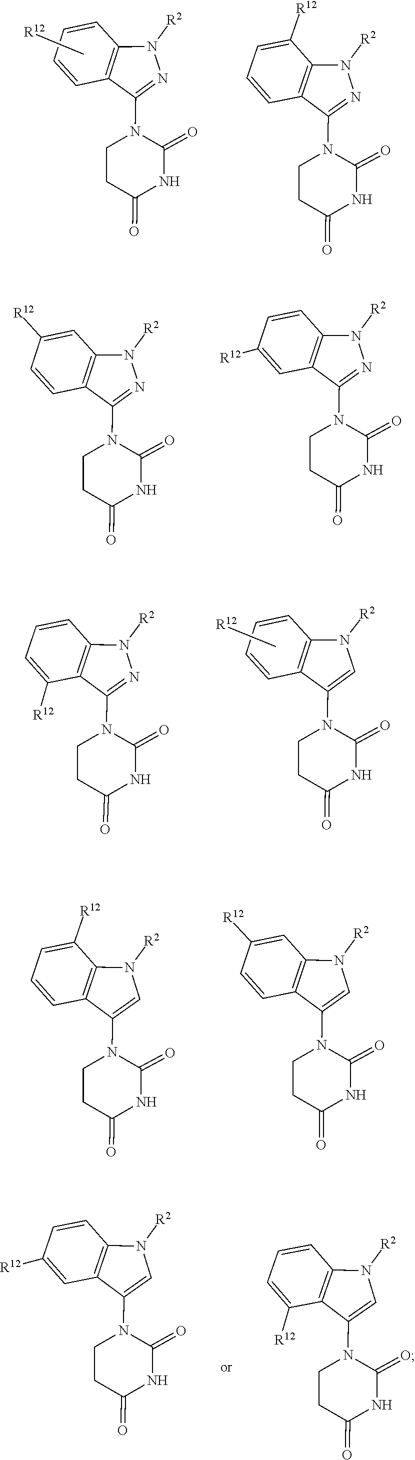

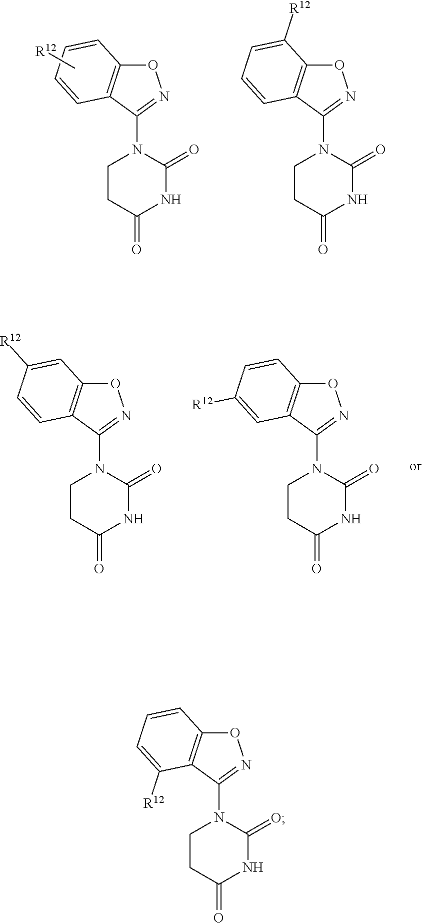

or a pharmaceutically acceptable salt thereof.

In certain embodiments the compound of the present invention is of Formula:

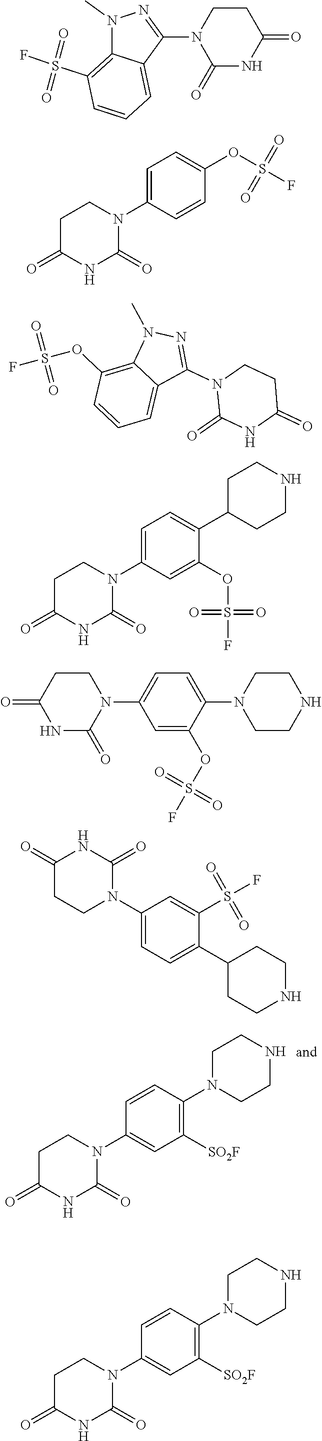

or a pharmaceutically acceptable salt thereof.

In certain embodiments the compound of the present invention is of Formula:

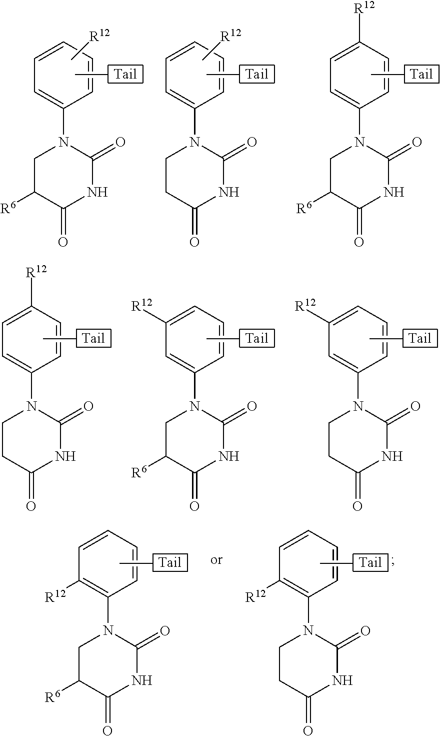

or a pharmaceutically acceptable salt thereof.

In certain embodiments the compound of the present invention is of Formula:

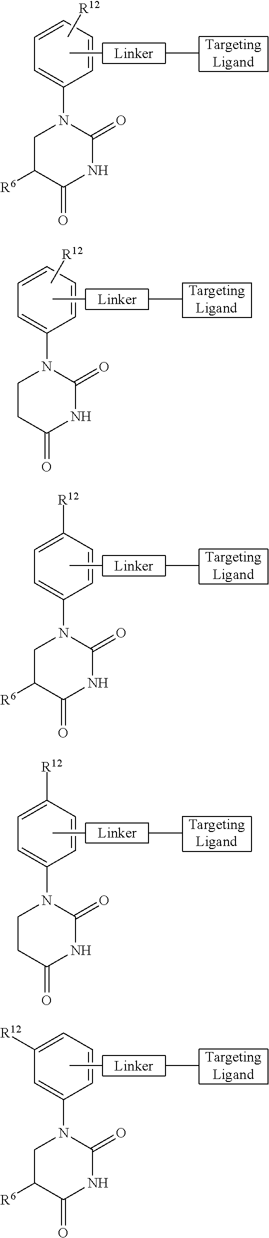

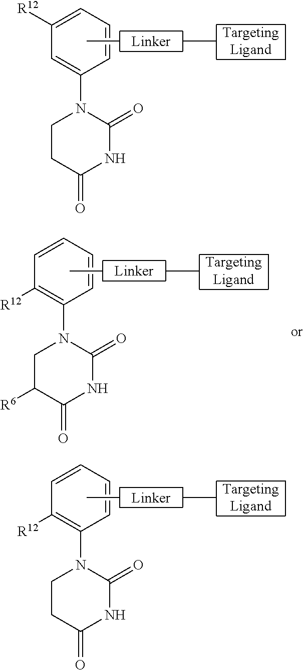

or a pharmaceutically acceptable salt thereof.

Non-limiting examples of compounds of Formula IVC include

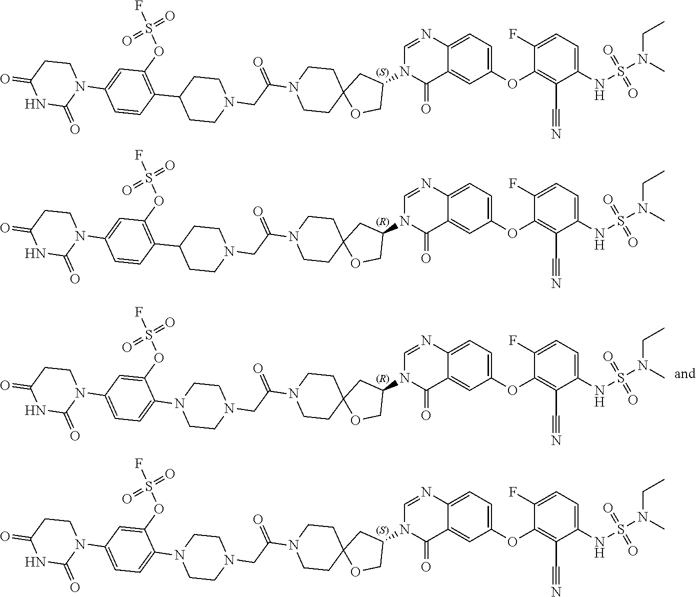

or a pharmaceutically acceptable salt thereof.

Additional non-limiting examples of the compound of the present invention include:

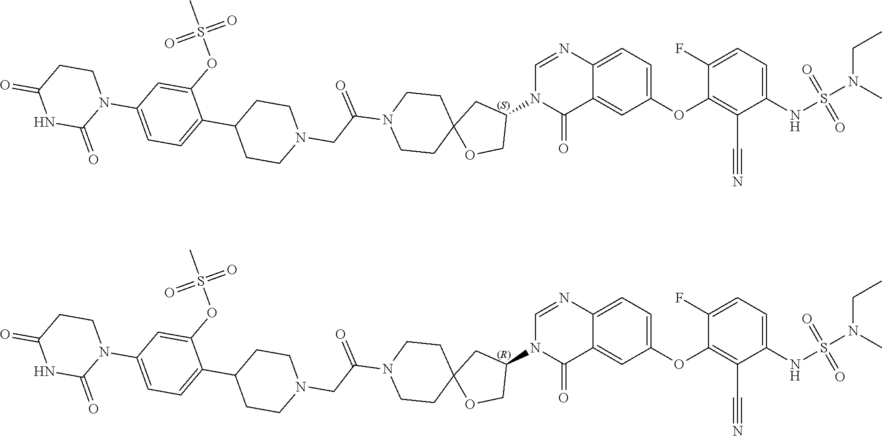

or a pharmaceutically acceptable salt thereof.

### Embodiments of Formula V

In certain embodiments the compound of the present invention is of Formula:

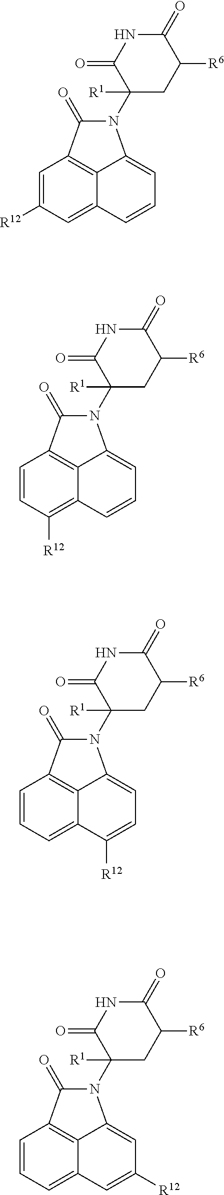

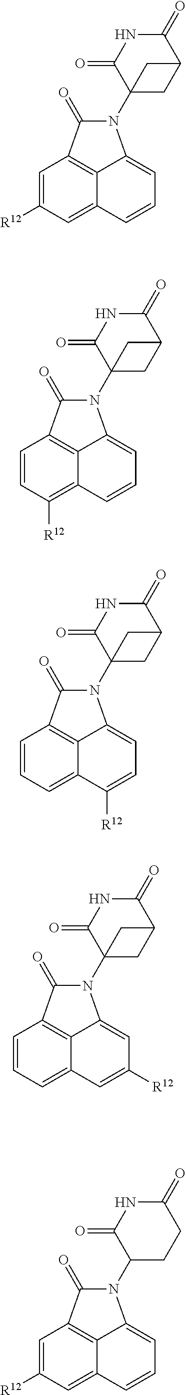

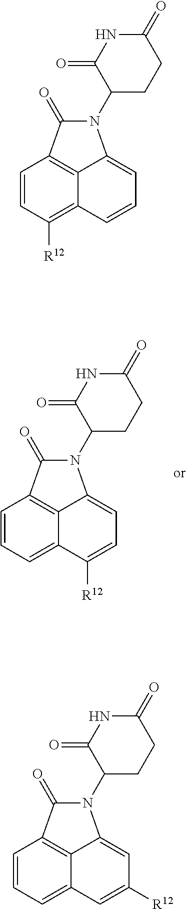

or a pharmaceutically acceptable salt thereof.

Non-limiting examples of compounds of Formula V include:

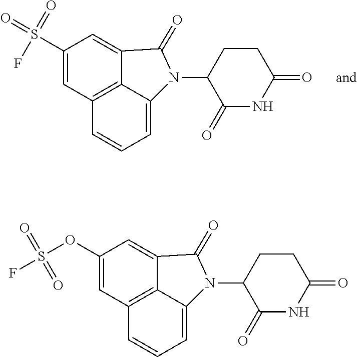 [SMILES: O=C1CCC(N2C(=O)c3cc(S(=O)(=O)F)cc4cccc2c34)C(=O)N1 ; O=C1CCC(N2C(=O)c3cc(OS(=O)(=O)F)cc4cccc2c34)C(=O)N1]

or a pharmaceutically acceptable salt thereof.

In certain embodiments the compound of the present invention is of Formula:

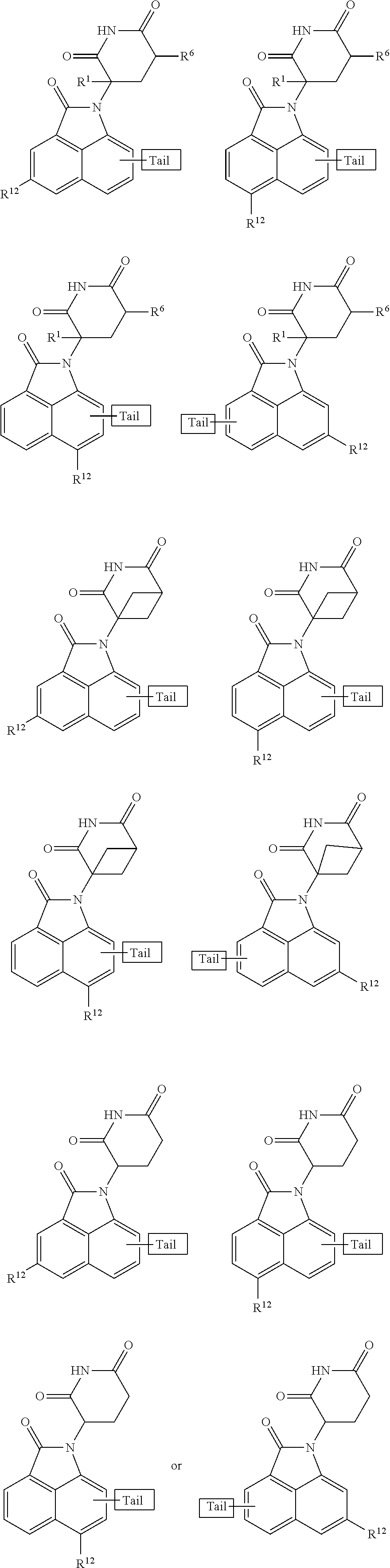

or a pharmaceutically acceptable salt thereof.

In certain embodiments the compound of the present invention is of Formula:

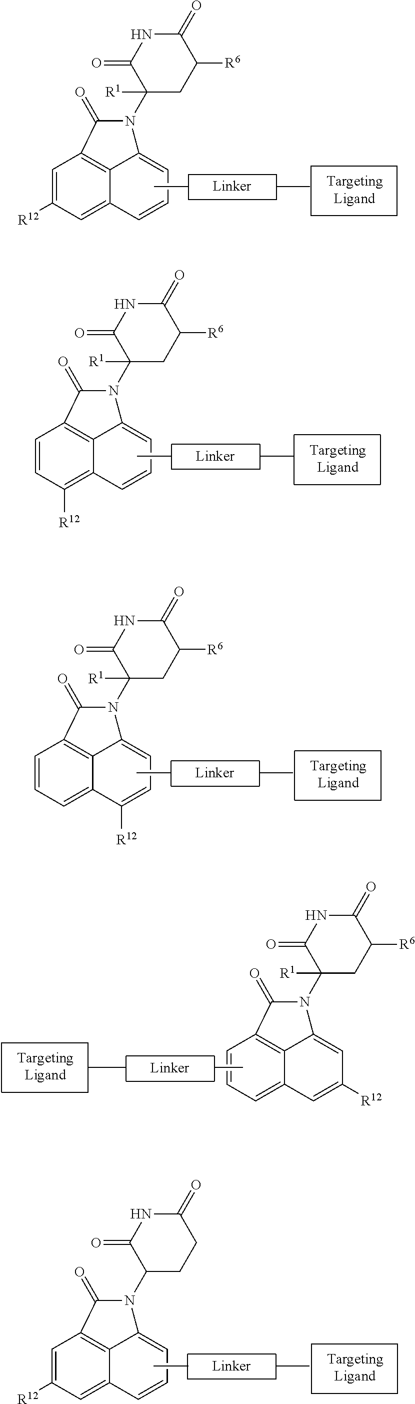

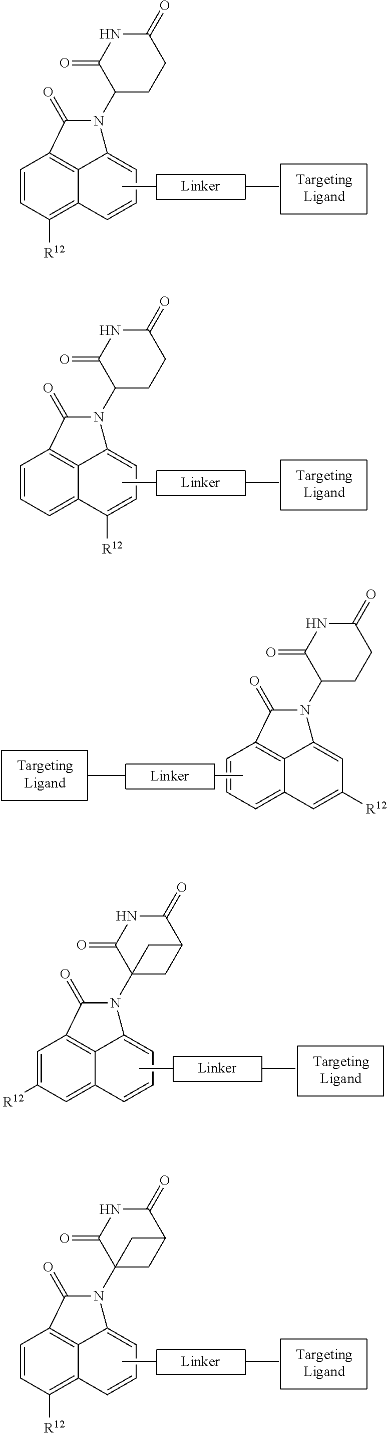

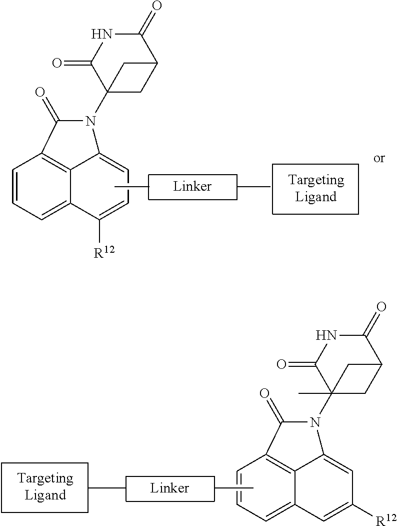

or a pharmaceutically acceptable salt thereof.

### Embodiments of Formula VI

In certain embodiments the compound of the present invention is of Formula:

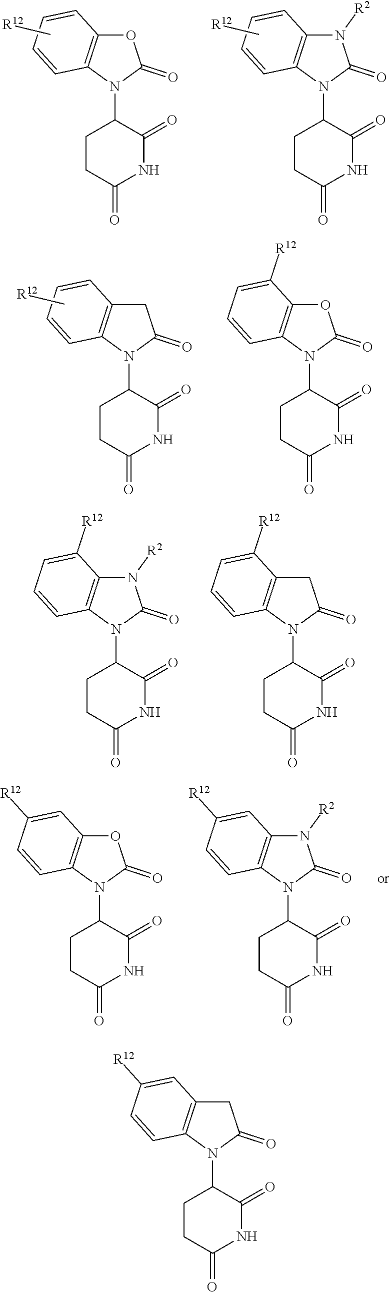

or a pharmaceutically acceptable salt thereof.

Non-limiting examples of compounds of Formula VI include:

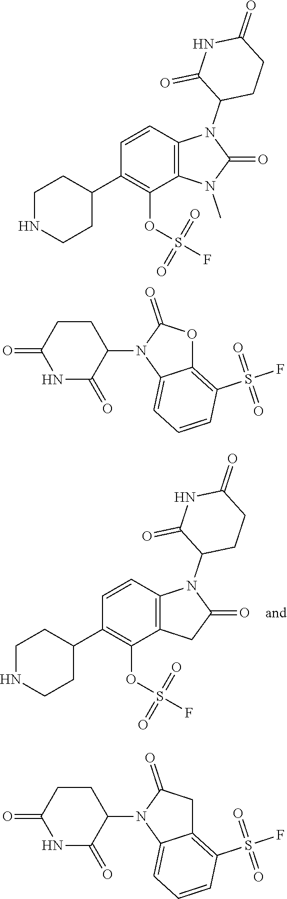

or a pharmaceutically acceptable salt thereof.

In certain embodiments the compound of the present invention is of Formula:

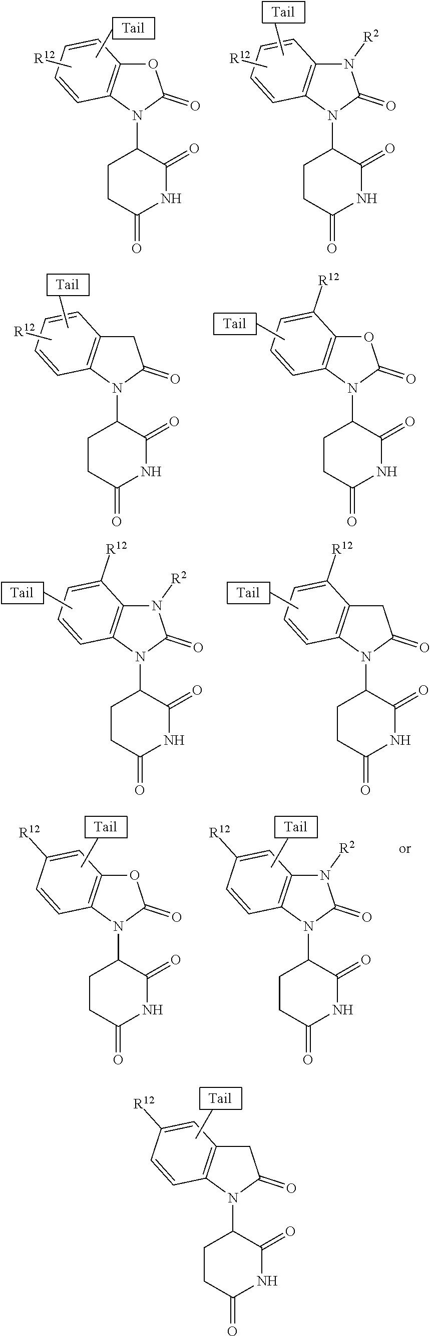

or a pharmaceutically acceptable salt thereof.

In certain embodiments the compound of the present invention is of Formula:

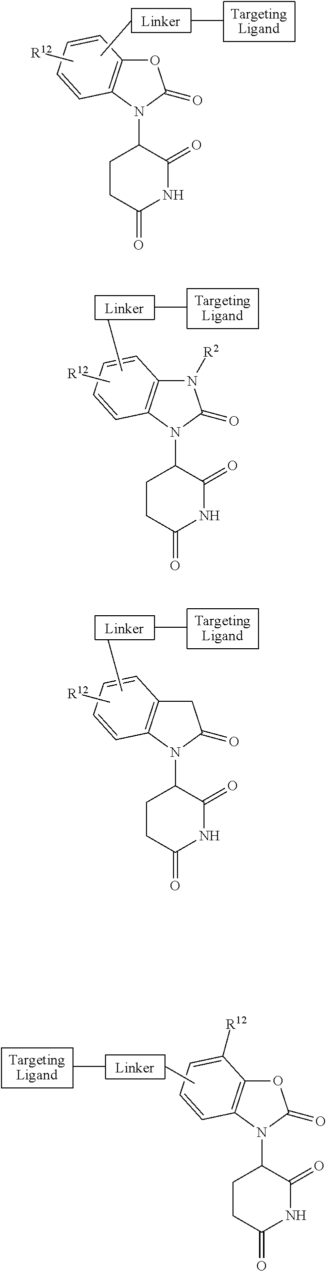

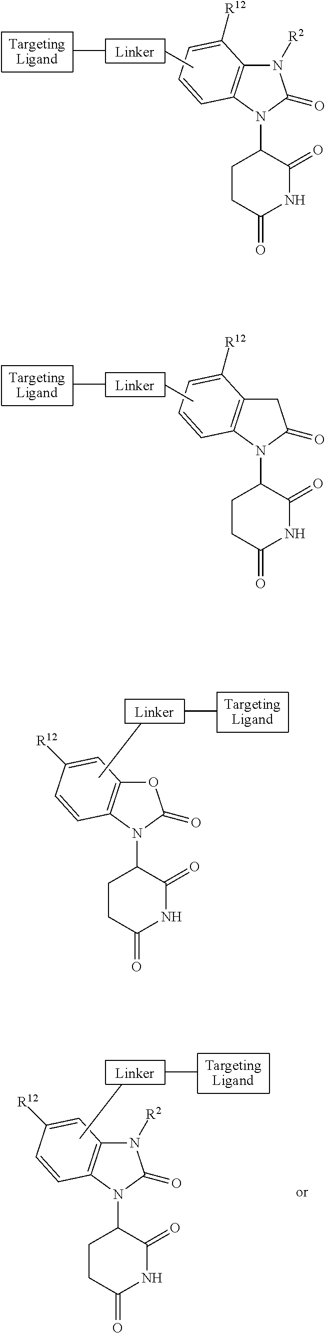

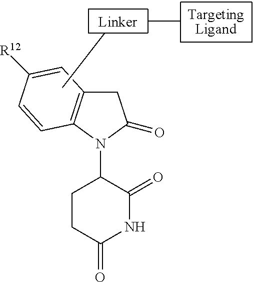

or a pharmaceutically acceptable salt thereof.

### Embodiments of Formula VII

In certain embodiments the compound of the present invention is of Formula:

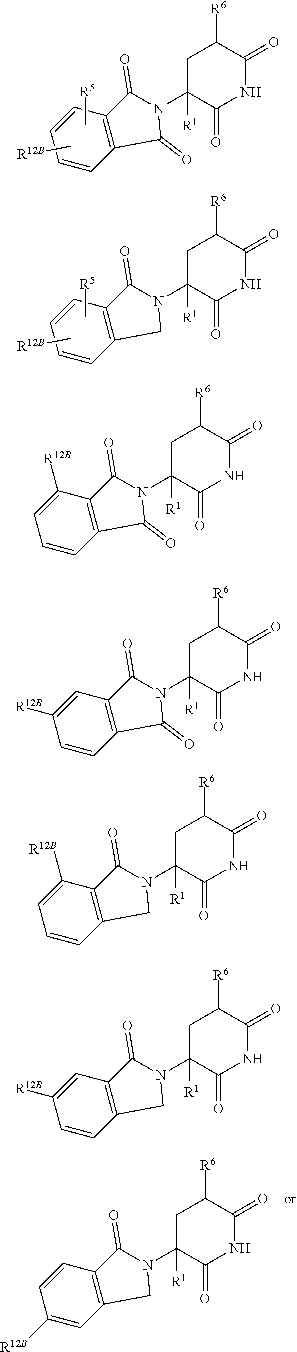

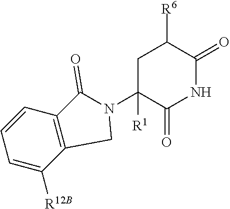

or a pharmaceutically acceptable salt thereof.

In certain embodiments the compound of the present invention is of Formula:

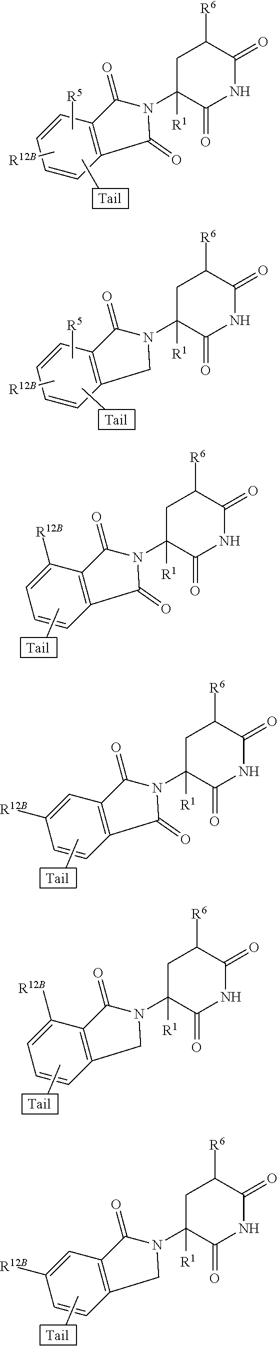

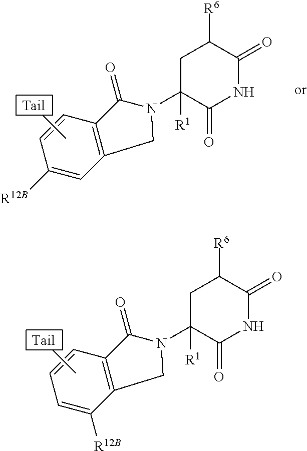

or a pharmaceutically acceptable salt thereof.

In certain embodiments the compound of the present invention is of Formula:

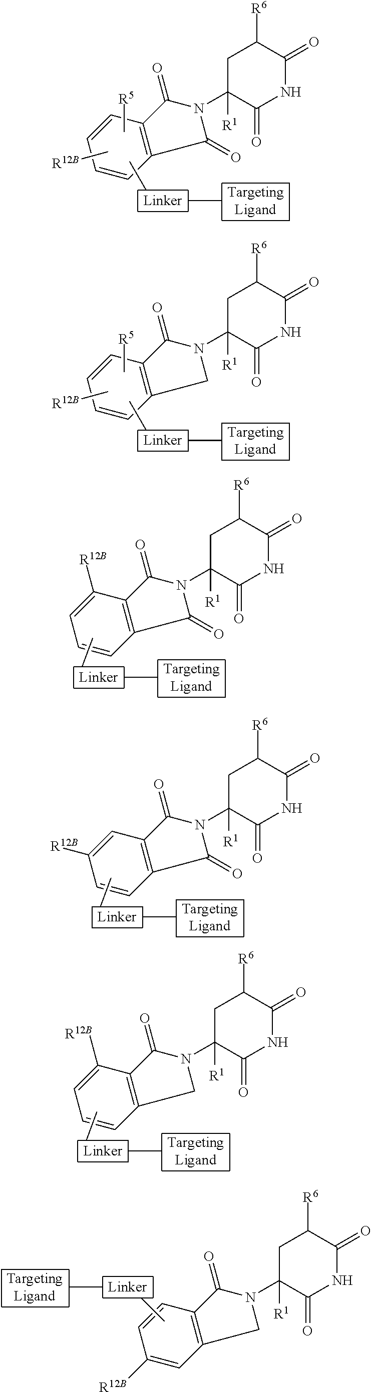

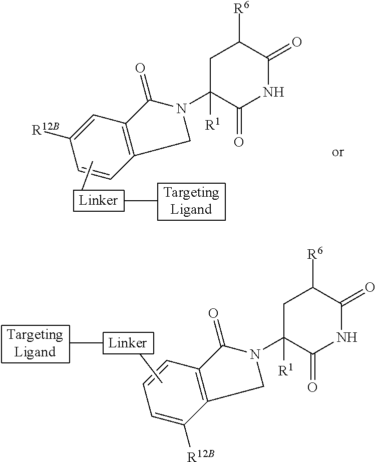

or a pharmaceutically acceptable salt thereof.

### Embodiments of Formula VIII

In certain embodiments the compound of the present invention is of Formula:

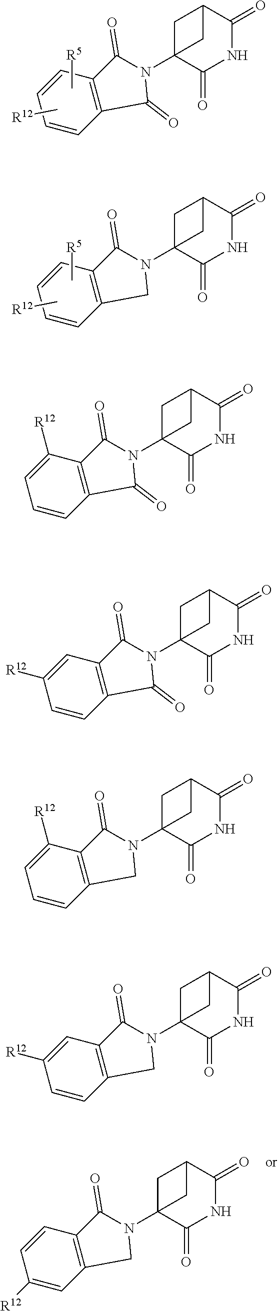

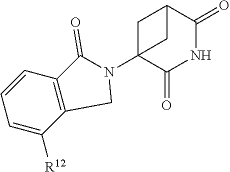

or a pharmaceutically acceptable salt thereof.

In certain embodiments the compound of the present invention is of Formula:

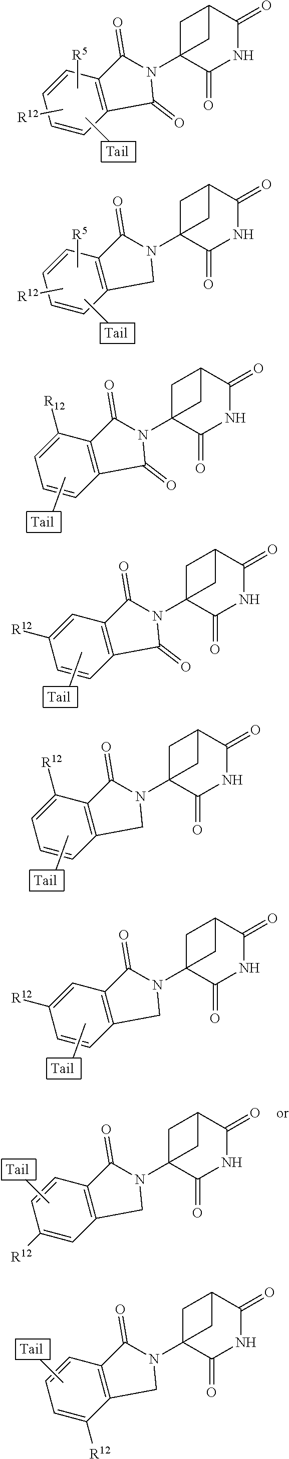

or a pharmaceutically acceptable salt thereof.

In certain embodiments the compound of the present invention is of Formula:

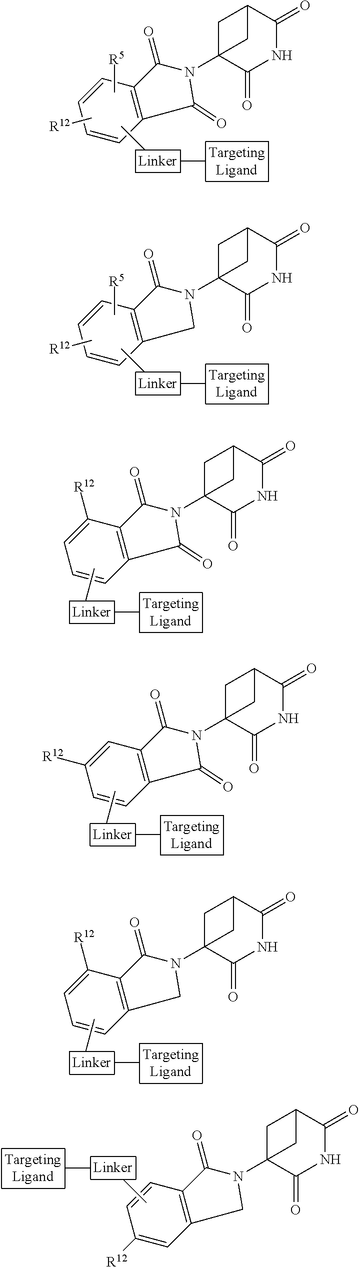

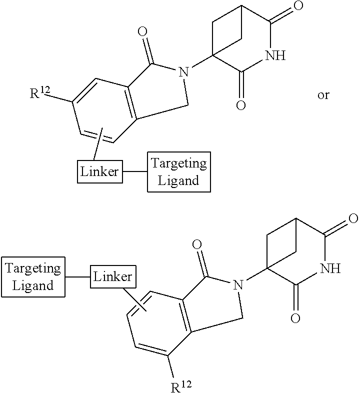

or a pharmaceutically acceptable salt thereof.

### Embodiments of Formula IX

In certain embodiments the compound of the present invention is of Formula:

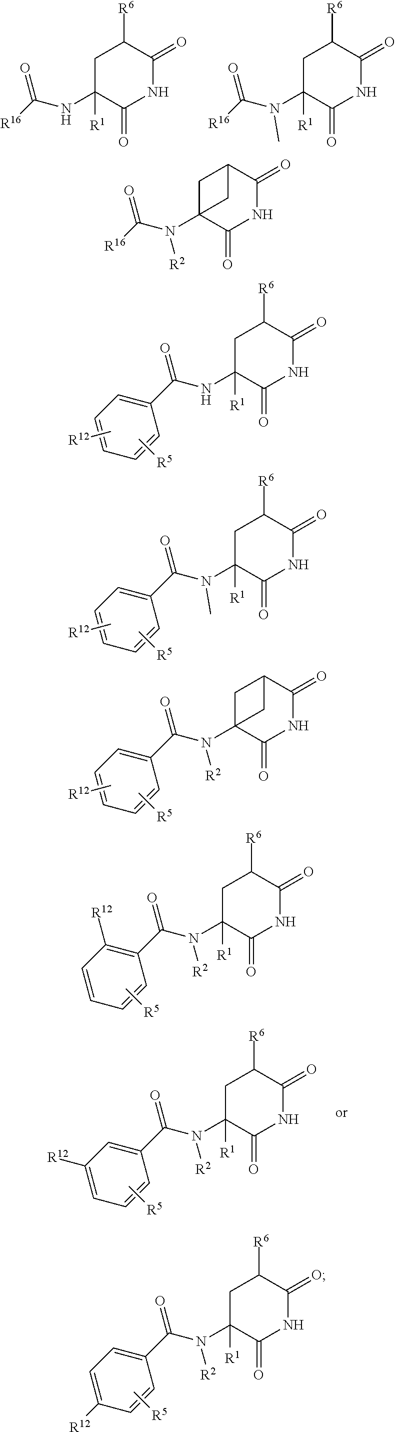

or a pharmaceutically acceptable salt thereof.

In certain embodiments the compound of the present invention is of Formula:

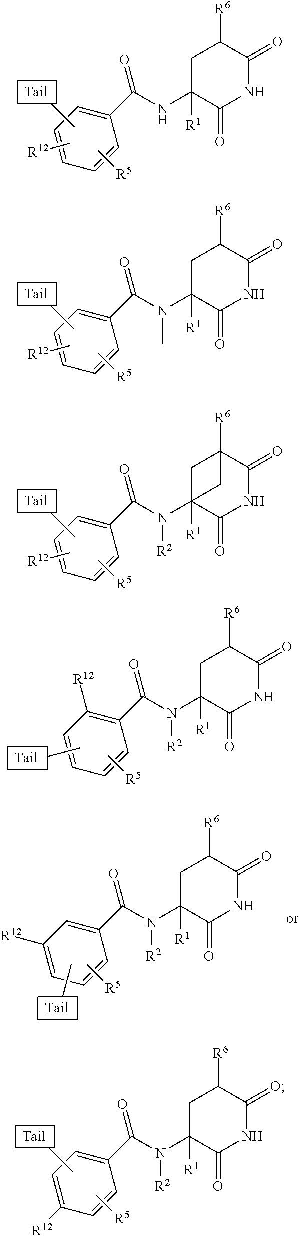

or a pharmaceutically acceptable salt thereof.

In certain embodiments the compound of the present invention is of Formula:

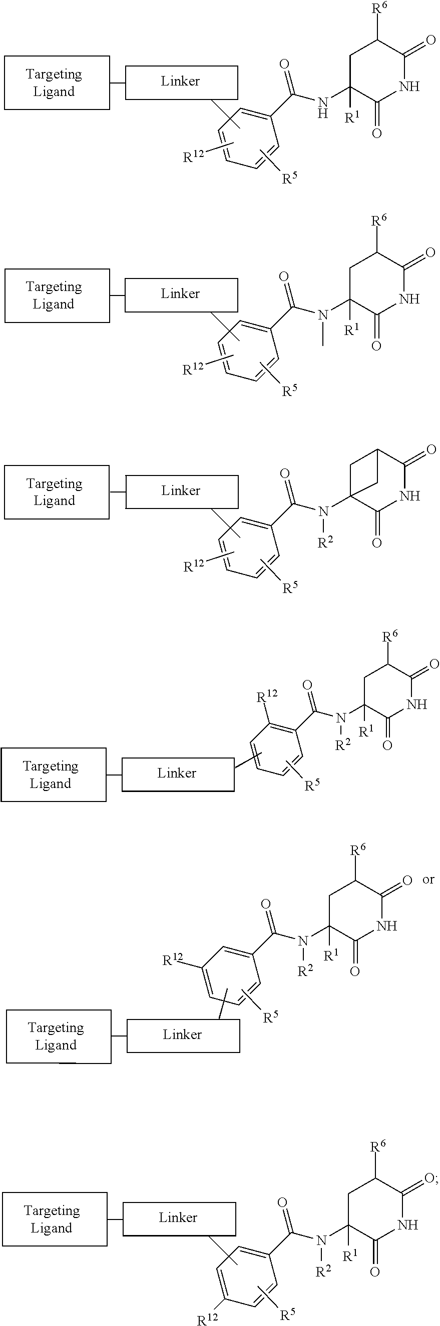

or a pharmaceutically acceptable salt thereof.

Covalent cereblon ligands with different structures are known in the art. For example, Cruite et al., published “Cereblon covalent modulation through structure-based design of histidine targeting chemical probes”, RSC Chemical Biol. 2022 3(9), 1105, describing electrophilic warheads that target histidine residues in a protein binding site of cereblon using sulfonyl exchange chemistry. See also, Cruite, et al, “Covalent stapling of the cereblon sensor loop histidine using sulfur-heterocycle exchange,” ACS Medicinal Chemistry Letters 2023 14(11), 1576; Nowak, et al. “Development of a covalent cereblon-based PROTAC employing a fluorosulfate warhead.” RSC Chemical Biol. 2023 4(11), 906; and Liu, et al. “Development of sulfonyl fluoride chemical probes to advance the discovery of cereblon modulators.” RSC Medicinal Chem. 2024 15(2), 607. Other covalent binders are described in WO 2023/056443 filed by Dana-Farber Cancer Institute and CN118344284 filed by Hangzhou Medical College.

### III. EMBODIMENTS OF THE PRESENT INVENTION

### Chirality Embodiments

The compounds of the present invention may have multiple stereocenters (e.g., chiral carbon atoms) including for example one or more stereocenters in the Degron (for example

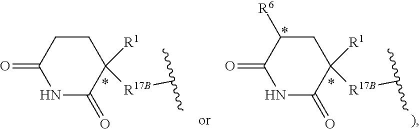

one or more stereocenters in the Linker (for example,

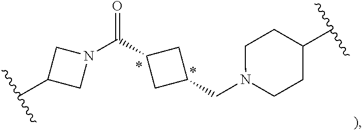 [SMILES: *C1CCN(C[C@H]2C[C@@H](C(=O)N3CC(*)C3)C2)CC1]

and/or one or more stereocenters in the Targeting Ligand.

In certain embodiments, the compound of the present invention is provided without regard to stereochemistry. In other embodiments, the compound of the present invention may have one or more chiral carbons presented in a stereochemically enriched (i.e., greater than about 50%, 60%, 70%, 80% or 90% pure) or even substantially pure form (greater than about 95%, 98% or 99% pure) of R and S stereochemistry. In certain aspects, the compound of the present invention has two stereochemically enriched and/or substantially pure stereocenters. In certain aspects, the two stereochemically enriched and/or substantially pure stereocenters are located in the ligase-binding moiety of the compound and the linker; or alternatively there are two in the Linker. In other aspects, there are three stereochemically enriched and/or substantially pure stereocenters, with one in the ligase-binding moiety of the compound and two in the linker. In yet other aspects, there are three stereochemically enriched and/or substantially pure stereocenters, with one in the ligase-binding moiety of the compound and two in the linker.

In certain embodiments, one stereocenter is in the R configuration and any others present are either stereochemically enriched or substantially pure. In certain embodiments, one stereocenter is in the S configuration and any others present are either stereochemically enriched or substantially pure.

In certain embodiments, one stereocenter is in the R configuration and any others present are without regard to stereochemistry, stereochemically enriched or substantially pure. In certain embodiments, one stereocenter is in the S configuration and any others present are without regard to stereochemistry, stereochemically enriched or substantially pure.

In certain embodiments, there is one stereocenter in the Degron and it is stereochemically enriched or substantially pure in the R-configuration. In other embodiments, there is one stereocenter in the Degron and it stereochemically enriched or substantially pure in the S-configuration.

In certain embodiments,

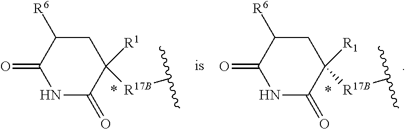

In certain embodiments,

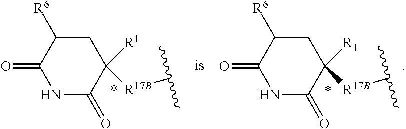

In certain embodiments,

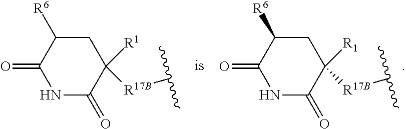

In certain embodiments,

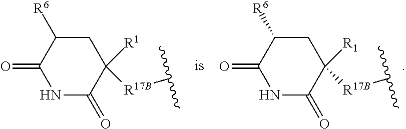

In certain embodiments,

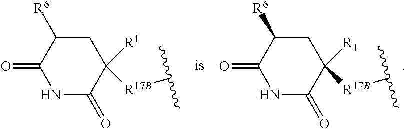

In certain embodiments,

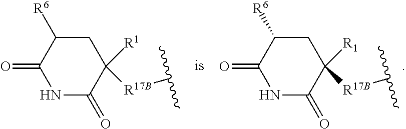

In certain embodiments, there is one stereocenter in the linker portion and it is a mixture of R- and S-configuration. In other embodiments, there is one stereocenter in the linker portion and it is stereochemically enriched or substantially pure R-configuration. In other embodiments, there is one stereocenter in the linker portion and it is stereochemically enriched or substantially pure S-configuration.

In certain embodiments, the linker contains one or more moieties with a chiral center. Non-limiting examples include heterocycle with a stereochemically enriched or substantially pure stereocenter for example piperidine with a substituent meta or ortho to the nitrogen or linking in the meta- or ortho- configuration; piperazine with a substituent or linking in the meta-or ortho- configuration; pyrrolidinone with or without a substituent; and pyrrolidine with or without a substituent.

Additional non-limiting examples of linker moieties with at least one chiral center include an alkyl with a stereochemically enriched or substantially pure stereocenter; an alkene with a stereochemically enriched or substantially pure stereocenter; an alkyne with a stereochemically enriched or substantially pure stereocenter; a haloalkyl with a stereochemically enriched or substantially pure stereocenter; an alkoxy with a stereochemically enriched or substantially pure stereocenter; and a cycloalkyl with a stereochemically enriched or substantially pure stereocenter

In certain embodiments, the linker includes

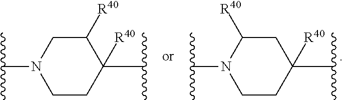

In certain embodiments, the linker includes

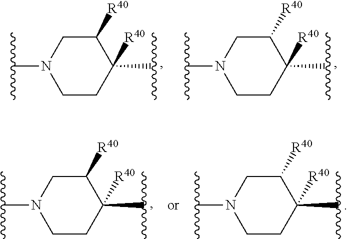

In certain embodiments, the linker includes

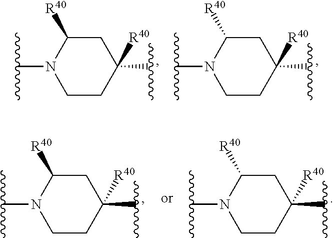

In certain embodiments, the linker includes

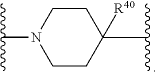

In certain embodiments, the linker includes

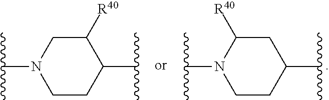

In certain embodiments, the linker includes

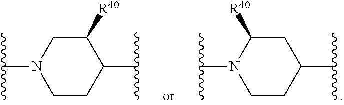

In certain embodiments, the linker includes

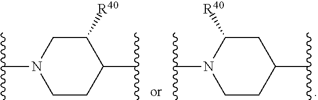

In certain embodiments, the linker includes

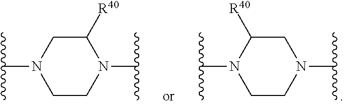

In certain embodiments, the linker includes

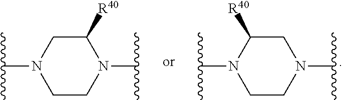

In certain embodiments, the linker includes

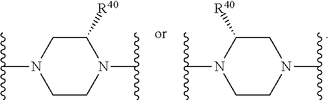

In certain embodiments, the linker includes

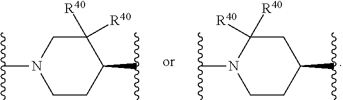

In certain embodiments, the linker includes

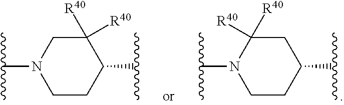

In certain embodiments, the linker includes

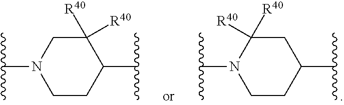

In certain embodiments, there is at least one stereocenter in the Targeting Ligand which is a mixture of R and S. In other embodiments, there is at least one stereocenter in the Targeting Ligand and it is stereochemically enriched or substantially pure in the R-configuration. In other embodiments, there is at least one stereocenter in the Targeting Ligand and it is stereochemically enriched or substantially pure in the S-configuration.

### Embodiments of alkyl

In certain embodiments, “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments, “alkyl” has one carbon.

In certain embodiments, “alkyl” has two carbons.

In certain embodiments, “alkyl” has three carbons.

In certain embodiments, “alkyl” has four carbons.

In certain embodiments, “alkyl” has five carbons.

In certain embodiments, “alkyl” has six carbons.

Non-limiting examples of “alkyl” include methyl, ethyl, propyl, butyl, pentyl, and hexyl.

Additional non-limiting examples of “alkyl” include isopropyl, isobutyl, isopentyl, and isohexyl.

Additional non-limiting examples of “alkyl” include sec-butyl, sec-pentyl, and sec-hexyl.

Additional non-limiting examples of “alkyl” include tert-butyl, tert-pentyl, and tert-hexyl.

Additional non-limiting examples of “alkyl” include neopentyl, 3-pentyl, and active pentyl.

### Embodiments of cycloalkyl

In certain embodiments, “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments, “cycloalkyl” has three carbons.

In certain embodiments, “cycloalkyl” has four carbons.

In certain embodiments, “cycloalkyl” has five carbons.

In certain embodiments, “cycloalkyl” has six carbons.

In certain embodiments, “cycloalkyl” has seven carbons.

In certain embodiments, “cycloalkyl” has eight carbons.

In certain embodiments, “cycloalkyl” has nine carbons.

In certain embodiments, “cycloalkyl” has ten carbons.

Non-limiting examples of “cycloalkyl” include cyclopropyl, cyclobutyl, cyclopentyl, cyclohexyl, cycloheptyl, cyclooctyl, and cyclodecyl.

### Embodiments of haloalkyl

In certain embodiments, “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments, “haloalkyl” has one carbon.

In certain embodiments, “haloalkyl” has one carbon and one halogen.

In certain embodiments, “haloalkyl” has one carbon and two halogens.

In certain embodiments, “haloalkyl” has one carbon and three halogens.

In certain embodiments, “haloalkyl” has two carbons.

In certain embodiments, “haloalkyl” has three carbons.

In certain embodiments, “haloalkyl” has four carbons.

In certain embodiments, “haloalkyl” has five carbons.

In certain embodiments, “haloalkyl” has six carbons.

Non-limiting examples of “haloalkyl” include,

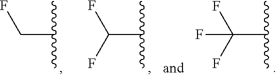

Additional non-limiting examples of “haloalkyl”

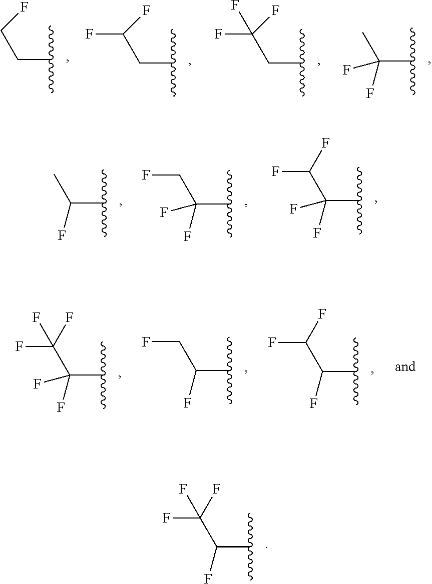

Additional non-limiting examples of “haloalkyl” include

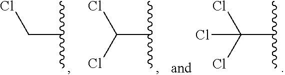

Additional non-limiting examples of “haloalkyl” include

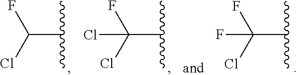

### Embodiments of Heterocycle

In certain embodiments, “heterocycle” refers to a cyclic ring with one nitrogen and 3, 4, 5, 6, 7, or 8 carbon atoms. In certain embodiments, “heterocycle” refers to a cyclic ring with one nitrogen and 3, 4, or 5 carbon atoms.

In certain embodiments, “heterocycle” refers to a cyclic ring with one nitrogen and one oxygen and 3, 4, 5, 6, 7, or 8 carbon atoms. In certain embodiments, “heterocycle” refers to a cyclic ring with one nitrogen and one oxygen and 4, 5, or 6 carbon atoms.

In certain embodiments, “heterocycle” refers to a cyclic ring with two nitrogen atoms and 3, 4, 5, 6, 7, or 8 carbon atoms. In certain embodiments, “heterocycle” refers to a cyclic ring with two nitrogen atoms and 4, 5, or 6 carbon atoms.

In certain embodiments, “heterocycle” refers to a cyclic ring with one oxygen and 3, 4, 5, 6, 7, or 8 carbon atoms. In certain embodiments, “heterocycle” refers to a cyclic ring with one oxygen and 3, 4, or 5 carbon atoms.

In certain embodiments, “heterocycle” refers to a cyclic ring with one sulfur and 3, 4, 5, 6, 7, or 8 carbon atoms. In certain embodiments, “heterocycle” refers to a cyclic ring with one sulfur and 3, 4, or 5 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocycle ring.

Non-limiting examples of “heterocycle” also include:

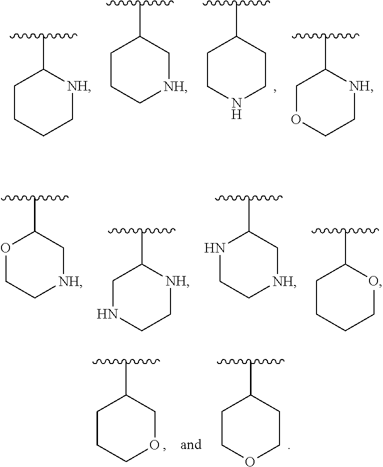

Additional non-limiting examples of “heterocycle” include:

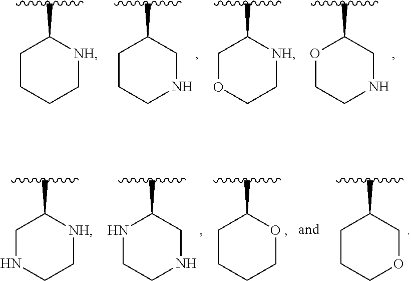

Additional non-limiting examples of “heterocycle” include:

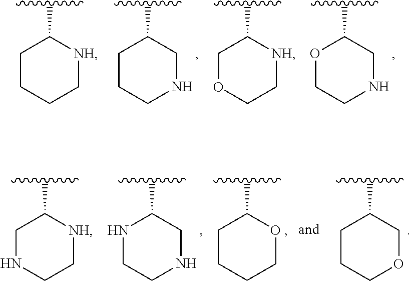

Non-limiting examples of “heterocycle” also include:

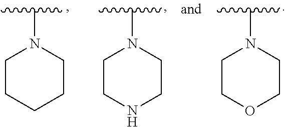

Non-limiting examples of “heterocycle” also include:

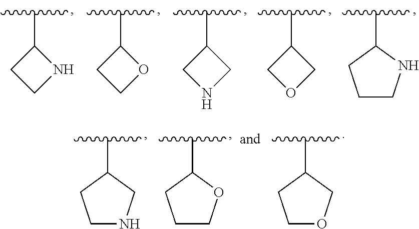

Additional non-limiting examples of “heterocycle” include:

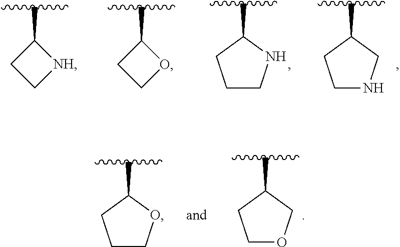 [SMILES: *[C@@H]1CCN1 ; *[C@@H]1CCO1 ; *[C@@H]1CCCN1 ; *[C@@H]1CCNC1 ; *[C@@H]1CCCO1 ; *[C@@H]1CCOC1]

Additional non-limiting examples of “heterocycle” include:

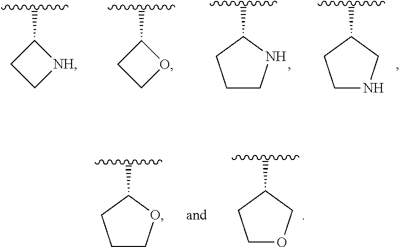 [SMILES: *[C@H]1CCN1 ; *[C@H]1CCO1 ; *[C@H]1CCCN1 ; *[C@H]1CCNC1 ; *[C@H]1CCCO1 ; *[C@H]1CCOC1]

### Embodiments of Heteroaryl

In certain embodiments, “heteroaryl” is a 5-membered aromatic group containing 1, 2, 3, or 4 nitrogen atoms.

Non-limiting examples of 5-membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5-membered “heteroaryl” groups include:

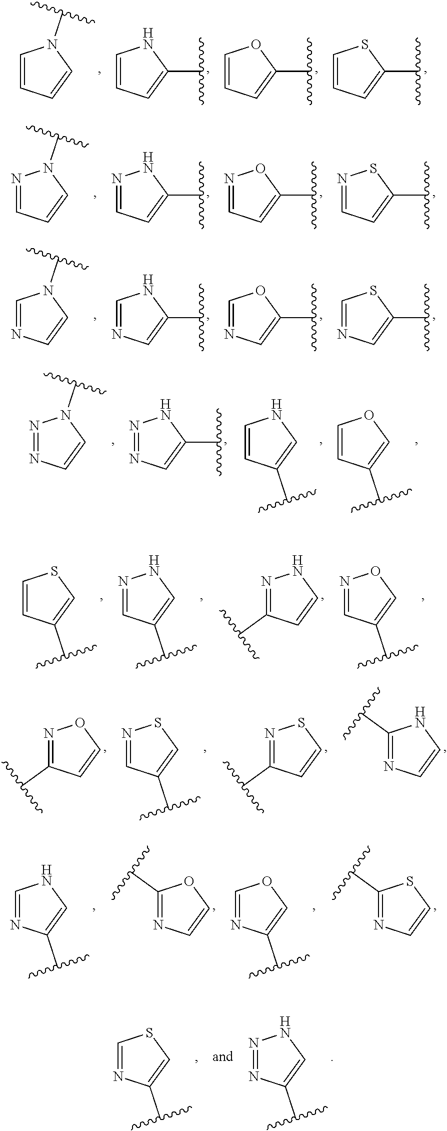

In certain embodiments, “heteroaryl” is a 6-membered aromatic group containing 1, 2, or 3 nitrogen atoms (i.e. pyridinyl, pyridazinyl, triazinyl, pyrimidinyl, and pyrazinyl).

Non-limiting examples of 6-membered “heteroaryl” groups with 1 or 2 nitrogen atoms include:

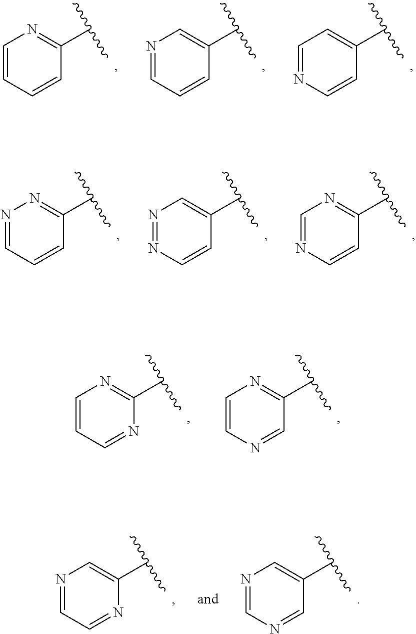

In certain embodiments, “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzoxazole, and benzothiazole.

Additional non-limiting examples of “heteroaryl” groups that are bicyclic include:

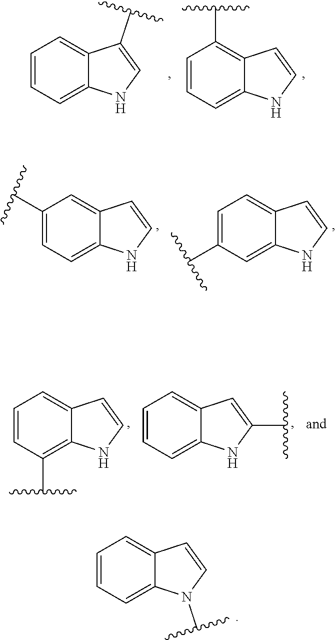

Additional non-limiting examples of “heteroaryl” groups that are bicyclic include:

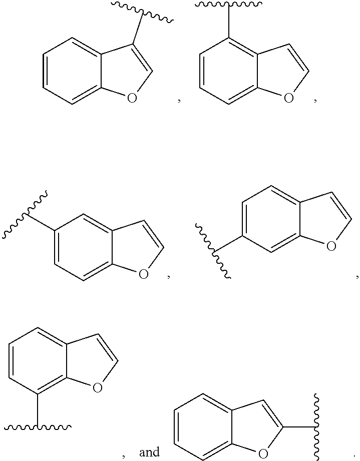 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

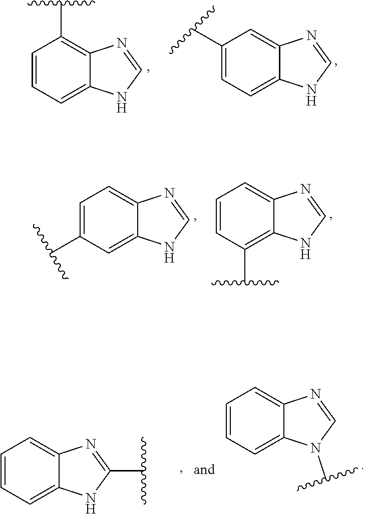

In certain embodiments, “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

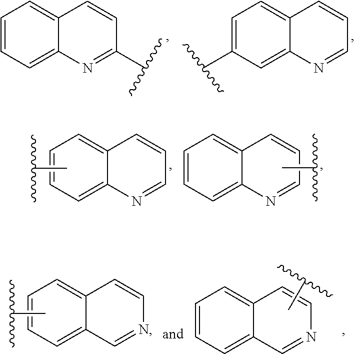

### Embodiments of Aryl

In certain embodiments, aryl is phenyl.

In certain embodiments, aryl is naphthyl.

### Embodiments of Bicycle

In certain embodiments, bicycle is

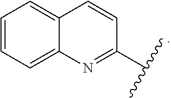 [SMILES: *c1ccc2ccccc2n1]

In certain embodiments, bicycle is

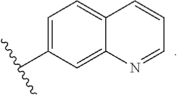 [SMILES: *c1ccc2cccnc2c1]

In certain embodiments, bicycle is

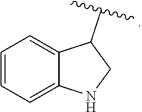 [SMILES: *C1CNc2ccccc21]

In certain embodiments, bicycle is

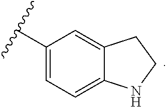 [SMILES: *c1ccc2c(c1)CCN2]

In certain embodiments, bicycle is

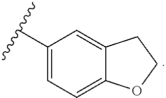 [SMILES: *c1ccc2c(c1)CCO2]

In certain embodiments, bicycle is

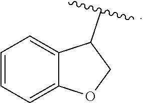 [SMILES: *C1COc2ccccc21]

In certain embodiments, bicycle is

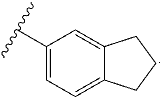 [SMILES: *c1ccc2c(c1)CCC2]

In certain embodiments, bicycle is

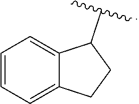 [SMILES: *C1CCc2ccccc21]

In certain embodiments, bicycle is a divalent group, for example, when R²² is bicycle.

In certain embodiments, bicycle is

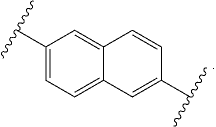 [SMILES: *c1ccc2cc(*)ccc2c1]

In certain embodiments, bicycle is

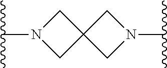 [SMILES: *N1CC2(C1)CN(*)C2]

In certain embodiments, bicycle is

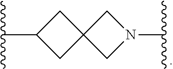 [SMILES: *C1CC2(C1)CN(*)C2]

In certain embodiments, bicycle is

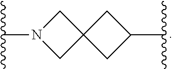 [SMILES: *C1CC2(C1)CN(*)C2]

In certain embodiments, bicycle is

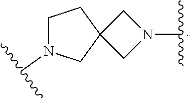 [SMILES: *N1CCC2(C1)CN(*)C2]

In certain embodiments, bicycle is

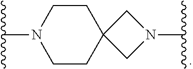 [SMILES: *N1CCC2(CC1)CN(*)C2]

In certain embodiments, bicycle is

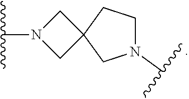 [SMILES: *N1CCC2(C1)CN(*)C2]

In certain embodiments, bicycle is

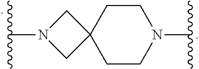 [SMILES: *N1CCC2(CC1)CN(*)C2]

In certain embodiments, bicycle is

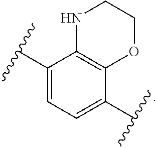 [SMILES: *c1ccc(*)c2c1NCCO2]

In certain embodiments, bicycle is

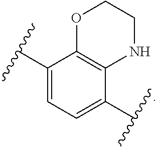 [SMILES: *c1ccc(*)c2c1NCCO2]

In certain embodiments, bicycle is

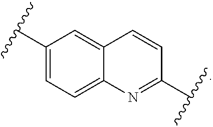 [SMILES: *c1ccc2nc(*)ccc2c1]

In certain embodiments, bicycle is

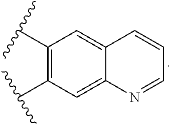 [SMILES: *c1cc2cccnc2cc1*]

In certain embodiments, bicycle is

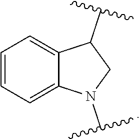 [SMILES: *C1CN(*)c2ccccc21]

In certain embodiments, bicycle is

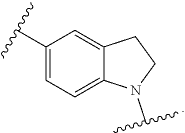 [SMILES: *c1ccc2c(c1)CCN2*]

In certain embodiments, bicycle is

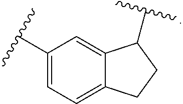 [SMILES: *c1ccc2c(c1)C(*)CC2]

In certain embodiments, bicycle is

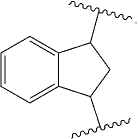 [SMILES: *C1CC(*)c2ccccc21]

In certain embodiments, bicycle is

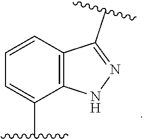 [SMILES: *c1n[nH]c2c(*)cccc12]

In certain embodiments, bicycle is

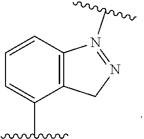

In certain embodiments, bicycle is

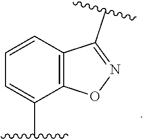 [SMILES: *c1noc2c(*)cccc12]

In certain embodiments, bicycle is

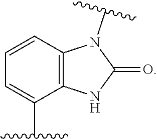 [SMILES: *c1cccc2c1[nH]c(=O)n2*]

### Embodiments of Optional Substituents

In certain embodiments, wherein a variable can be optionally substituted, it is not substituted.

In certain embodiments, wherein a variable can be optionally substituted, it is substituted with 1 substituent.

In certain embodiments, wherein a variable can be optionally substituted, it is substituted with 2 substituents.

In certain embodiments, wherein a variable can be optionally substituted, it is substituted with 3 substituents.

In certain embodiments, wherein a variable can be optionally substituted, it is substituted with 4 substituents.

### Embodiments of Linker

Linker as described herein can be drawn in either direction, i.e., either the left end is linked to Degron and the right end to the Targeting Ligand, or the left end is linked to the Targeting Ligand and the right end is linked to the Degron. In certain embodiments the left end of Linker is linked to the Degron. In other embodiments the left end of Linker is linked to the Targeting Ligand.

In certain embodiments, Linker is a bond.

In some embodiments, Linker is a moiety selected from Formula LI, Formula LII, Formula LIII, Formula LIV, Formula LV, Formula LVI, and Formula LVII:

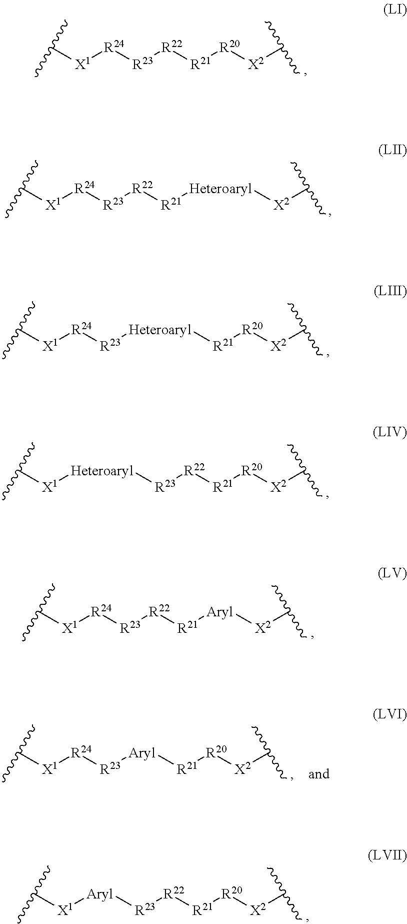

wherein all variables are defined as above.

In other embodiments, the Linker is a moiety selected from Formula LVIII, LIX, and LX:

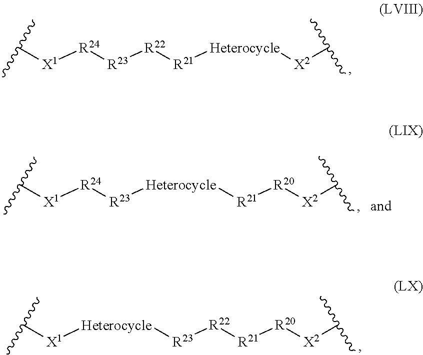

wherein all variables are defined as above.

In other embodiments of LVIII, LIX and LX, a carbocyclic ring is used in place of the heterocycle.

In certain embodiments, the Linker has a chain of 2 to 14, 15, 16, 17, 18 or 20 or more carbon atoms of which one or more carbons can be replaced by a heteroatom such as O, N, S, or P.

In certain embodiments, the chain has 2, 3, 4, 5, 6, 7, 8, 9, 10, 11, 12, 13, 14, 15, 16, 17, 18, 19 or 20 contiguous atoms in the chain. For example, the chain may include 1 or more ethylene glycol units that can be contiguous, partially contiguous or non-contiguous (for example, 2, 3, 4, 5, 6, 7, 8, 9, 10, 11 or 12 ethylene glycol units).

In certain embodiments, the chain has at least 1, 2, 3, 4, 5, 6, 7, or 8 contiguous chains which can have branches which can be independently alkyl, aryl, heteroaryl, alkenyl, or alkynyl, cycloalkyl or heterocycle substituents.

In other embodiments, the linker can include or be comprised of one or more of ethylene glycol, propylene glycol, lactic acid and/or glycolic acid. Lactic acid segments tend to have a longer half-life than glycolic acid segments. Block and random lactic acid-co-glycolic acid moieties, as well as ethylene glycol and propylene glycol, are known in the art to be pharmaceutically acceptable and can be modified or arranged to obtain the desired half-life and hydrophilicity. In certain aspects, these units can be flanked or interspersed with other moieties, such as alkyl, aryl, heteroaryl, heterocycle, cycloalkyl, etc., as desired to achieve the appropriate drug properties.

The following are non-limiting examples of Linkers that can be used in this invention. Based on this elaboration, those of skill in the art will understand how to use the full breadth of Linkers that will accomplish the goal of the invention.

As certain non-limiting examples, Formula LI, Formula LII, Formula LIII, Formula LIV, Formula LV, Formula LVI, or Formula LVII include:

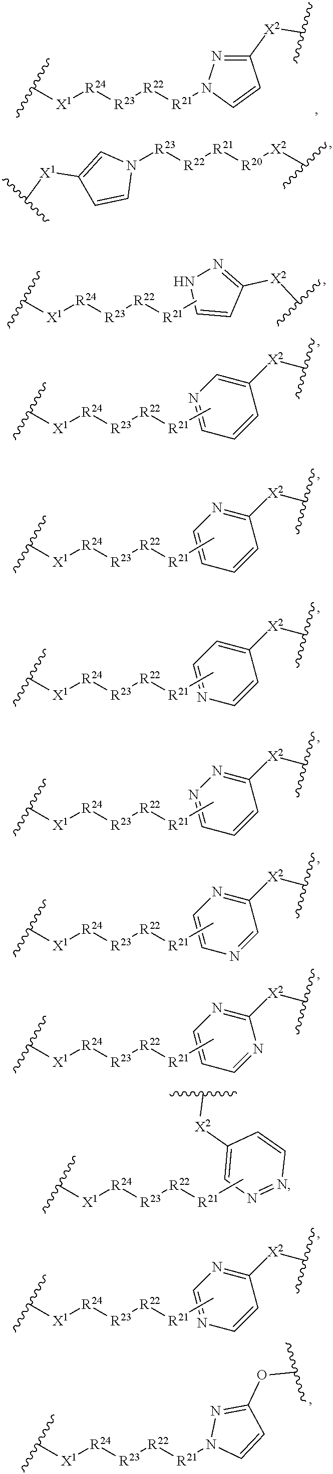

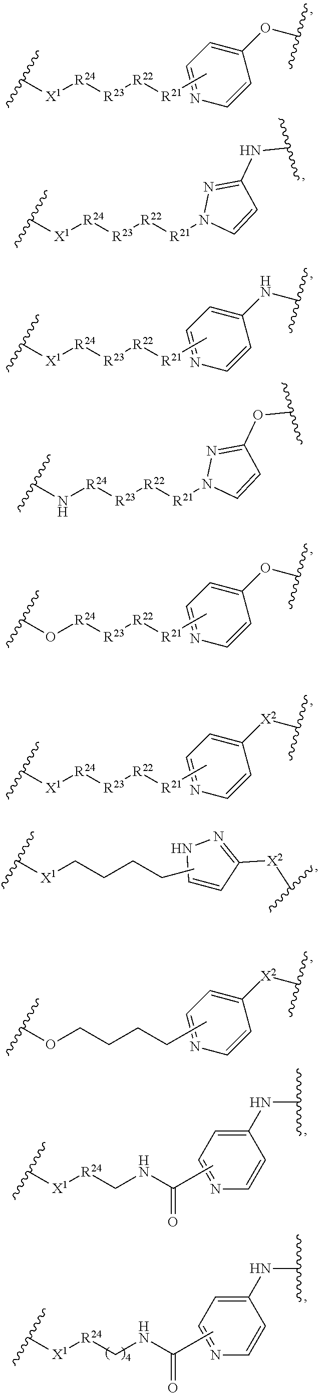

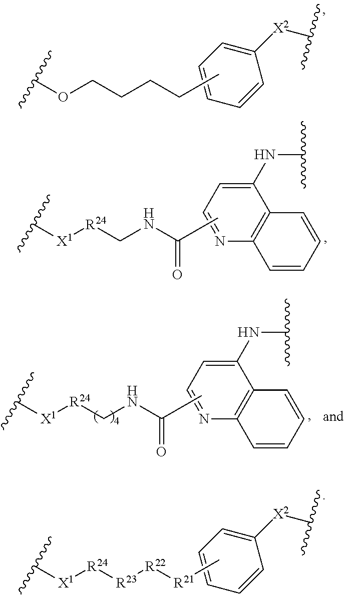

In additional embodiments Linker is selected from:

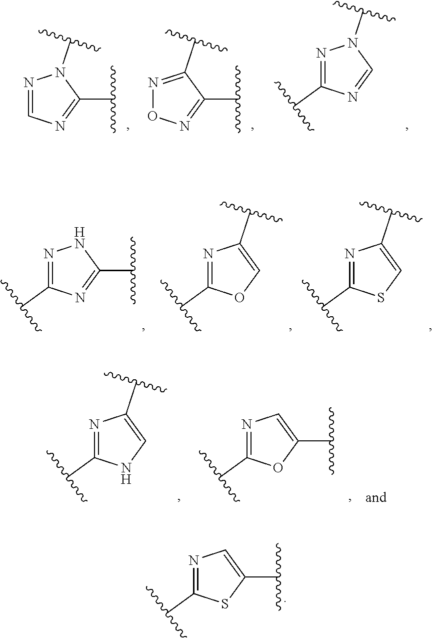

In additional embodiments Linker is selected from:

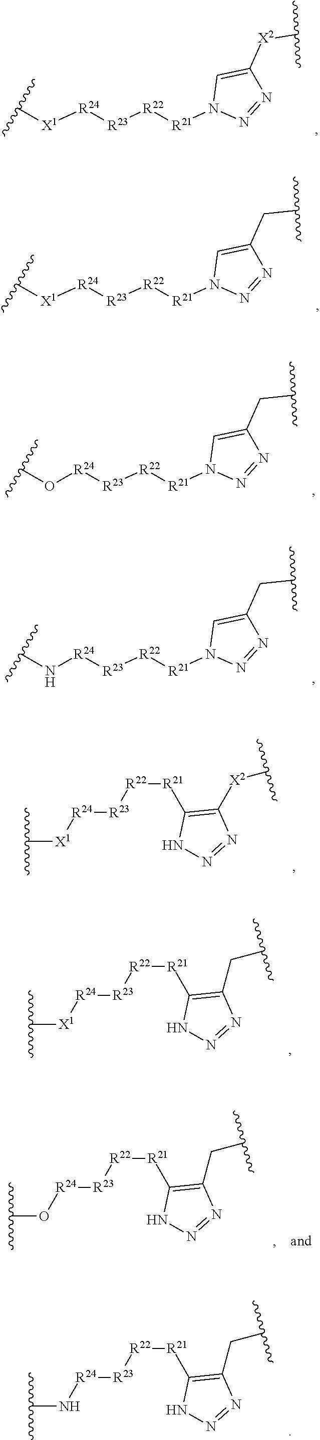

In one embodiment X¹ is attached to the Targeting Ligand. In another embodiment X² is attached to the Targeting Ligand.

Non-limiting examples of moieties of R²⁰, R²¹, R²², R²³, and R²⁴ include:

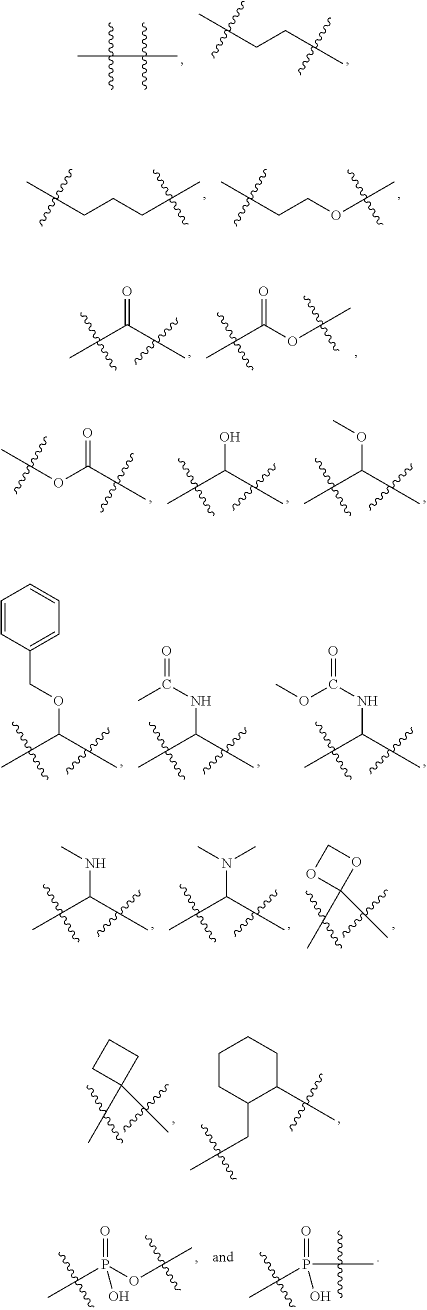

Additional non-limiting examples of moieties of R²⁰, R²¹, R²² R²³ and R²⁴ include:

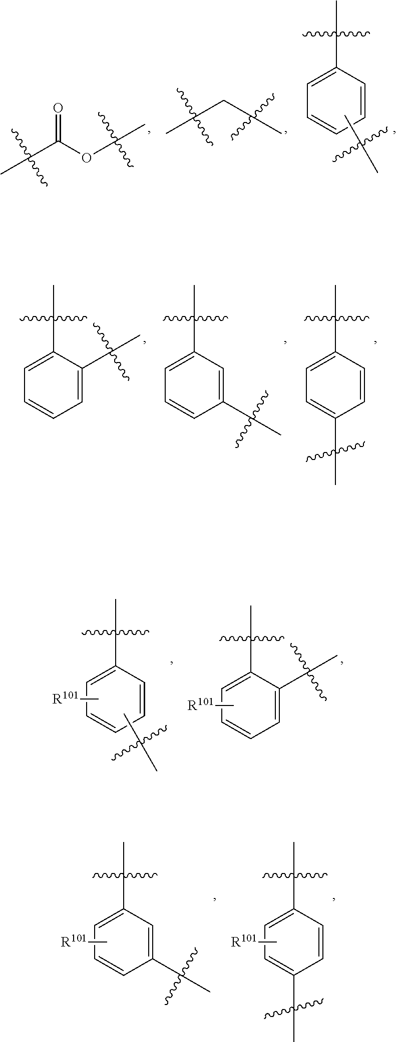

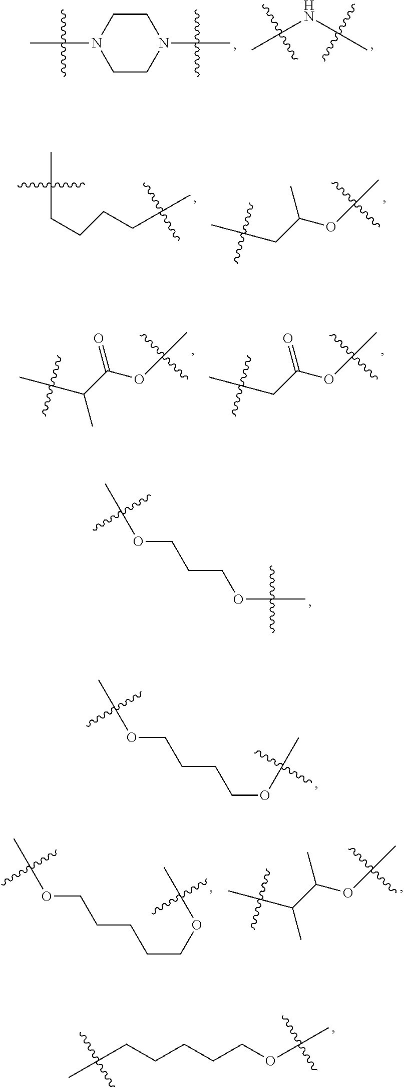

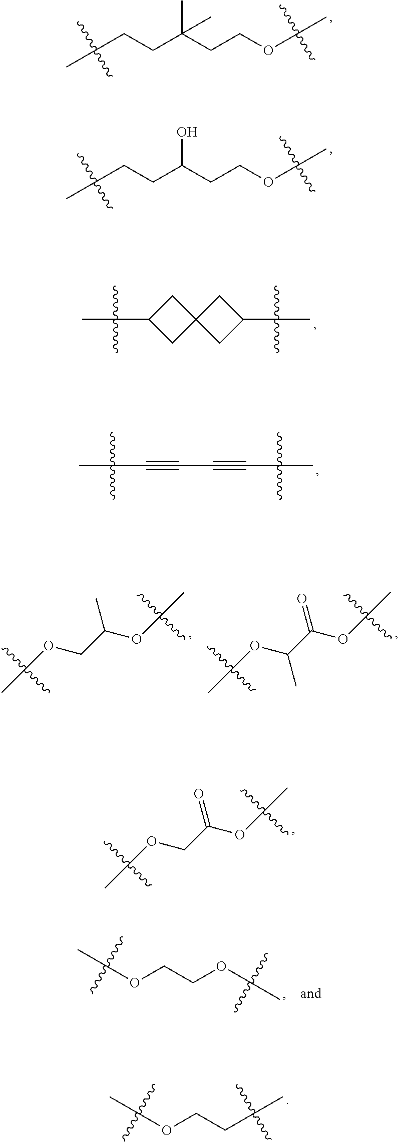

Additional non-limiting examples of moieties of R²⁰, R²¹, R²², R²³, and R²⁴ include:

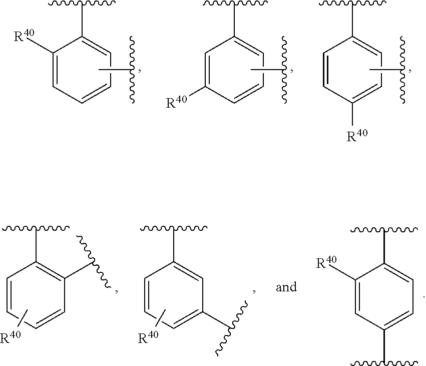

Additional non-limiting examples of moieties of R²⁰, R²¹, R²², R²³, and R²⁴ include:

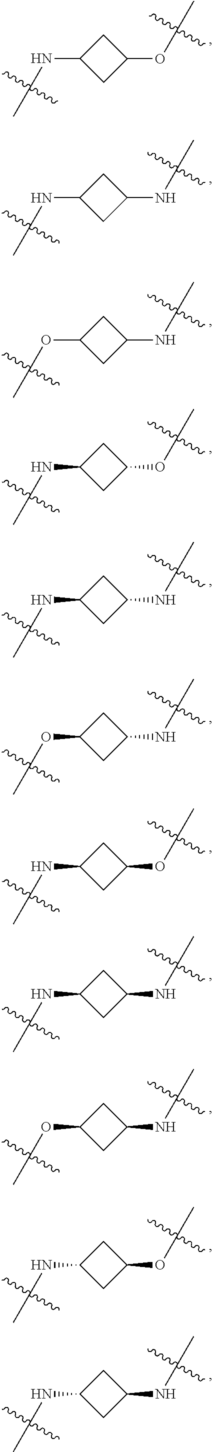

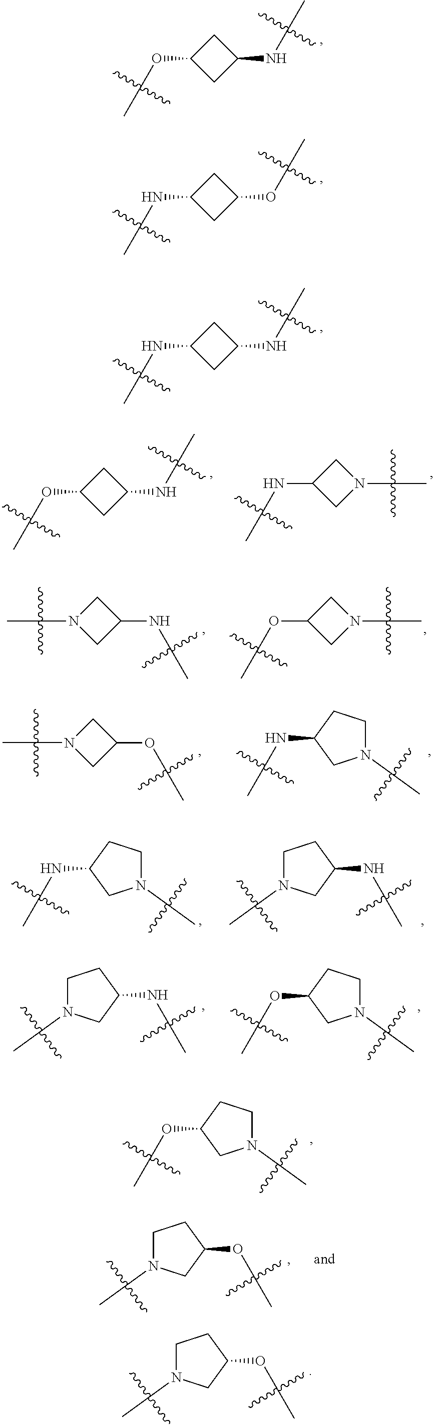

In additional embodiments, the Linker moiety is an optionally substituted (poly)ethylene glycol having at least 1, at least 2, at least 3, at least 4, at least 5, at least 6, at least 7, at least 8, at least 9, at least 10, ethylene glycol units, or optionally substituted alkyl groups interspersed with optionally substituted, O, N, S, P or Si atoms. In certain embodiments, Linker is flanked, substituted, or interspersed with an aryl, phenyl, benzyl, alkyl, alkylene, or heterocycle group. In certain embodiments, Linker may be asymmetric or symmetrical. In some embodiments, Linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units. In any of the embodiments of the compounds described herein, Linker group may be any suitable moiety as described herein.

In additional embodiments, Linker is selected from the group consisting of:

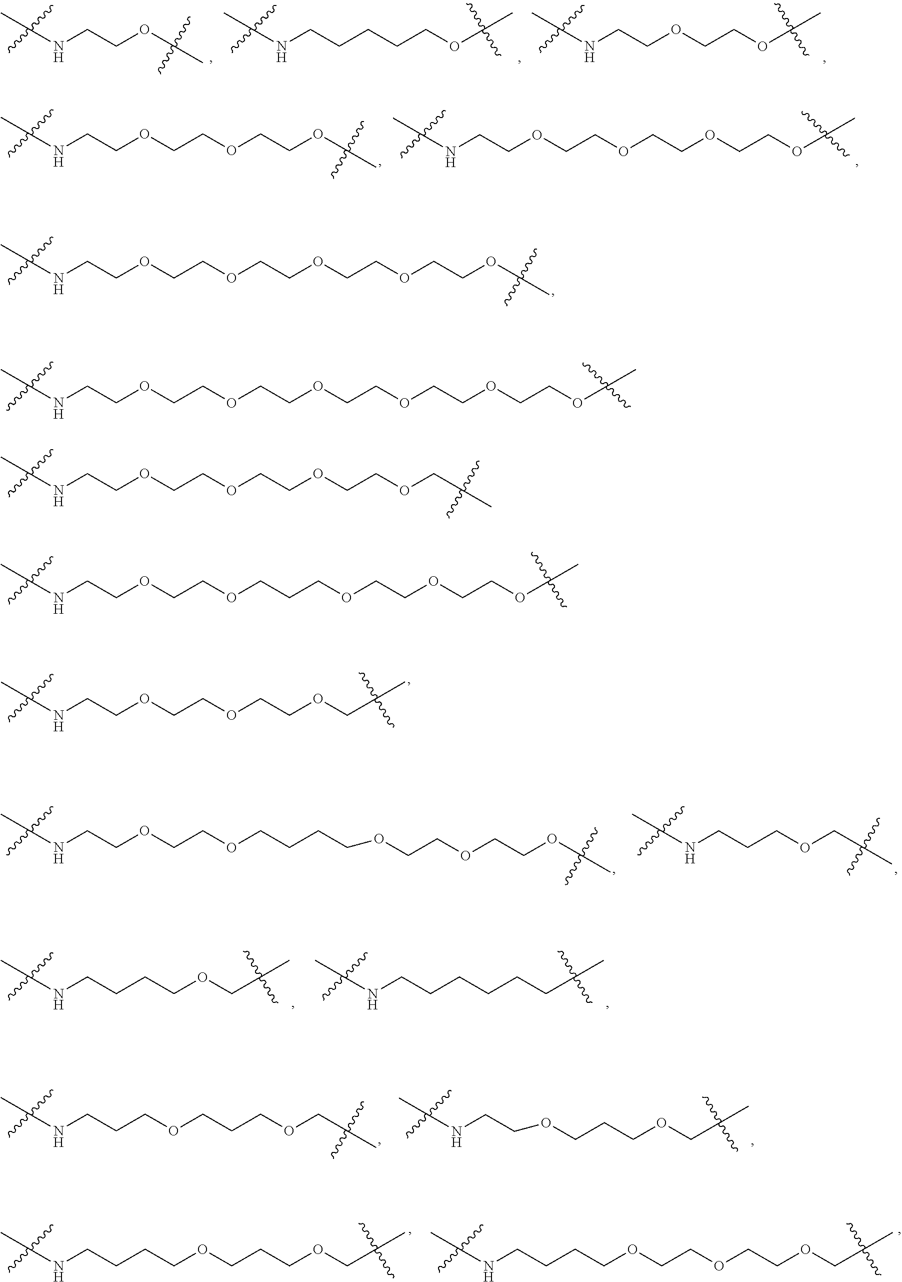

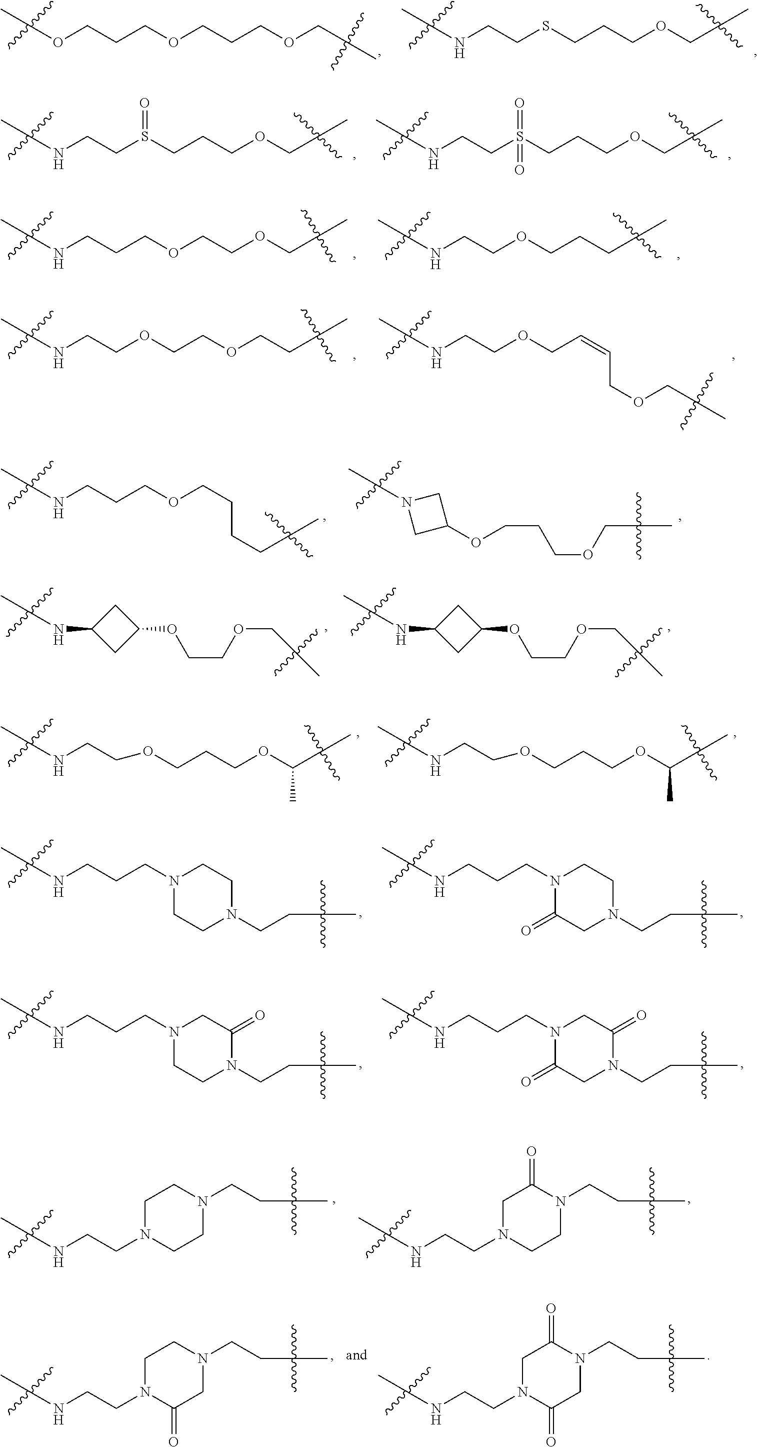

In additional embodiments, Linker is selected from the group consisting of:

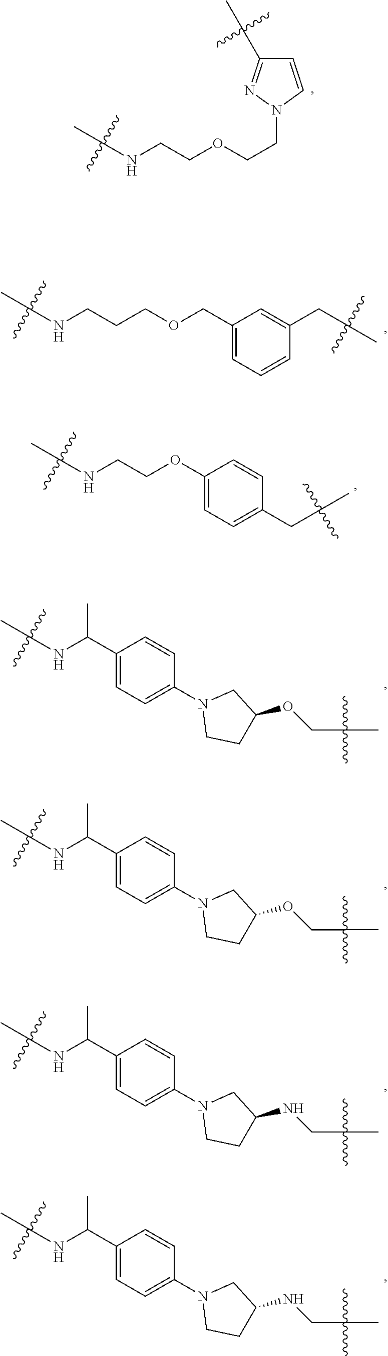

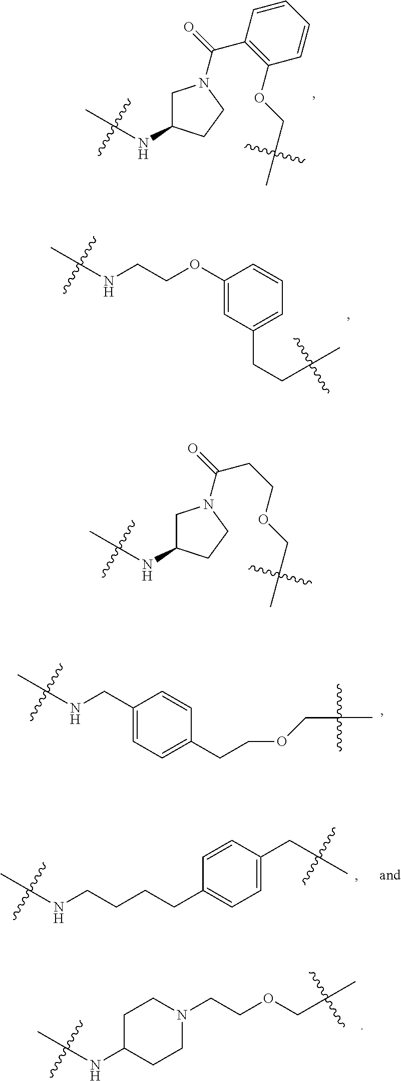 [SMILES: *COc1ccccc1C(=O)N1CC[C@@H](N*)C1 ; *CCc1cccc(OCCN*)c1 ; *COCCC(=O)N1CC[C@@H](N*)C1 ; *COCCc1ccc(CN*)cc1 ; *Cc1ccc(CCCCN*)cc1 ; *COCCN1CCC(N*)CC1]

In additional embodiments, Linker is selected from the group consisting of:

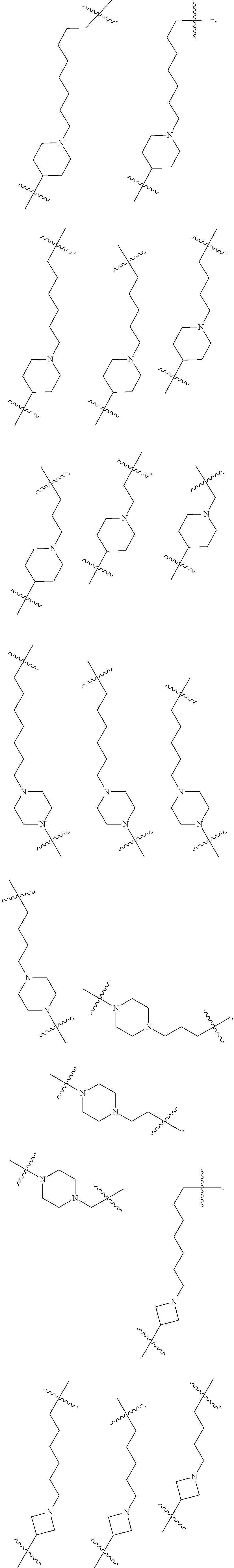

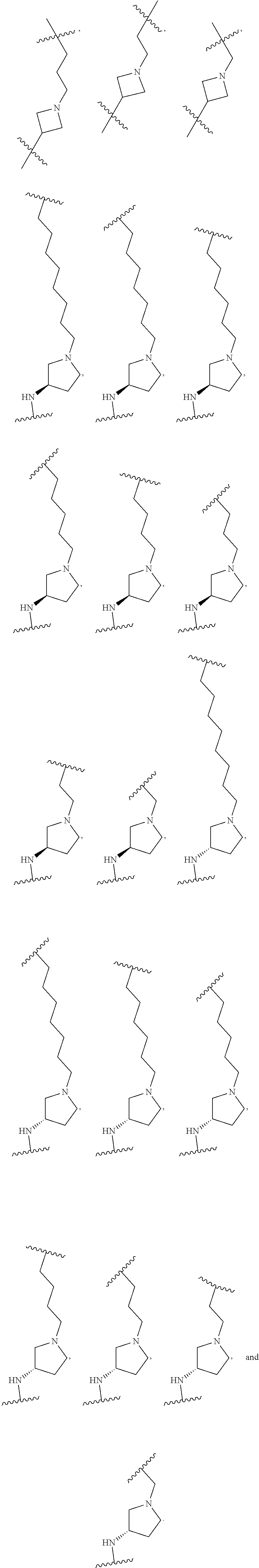

In additional embodiments, Linker is selected from the group consisting of:

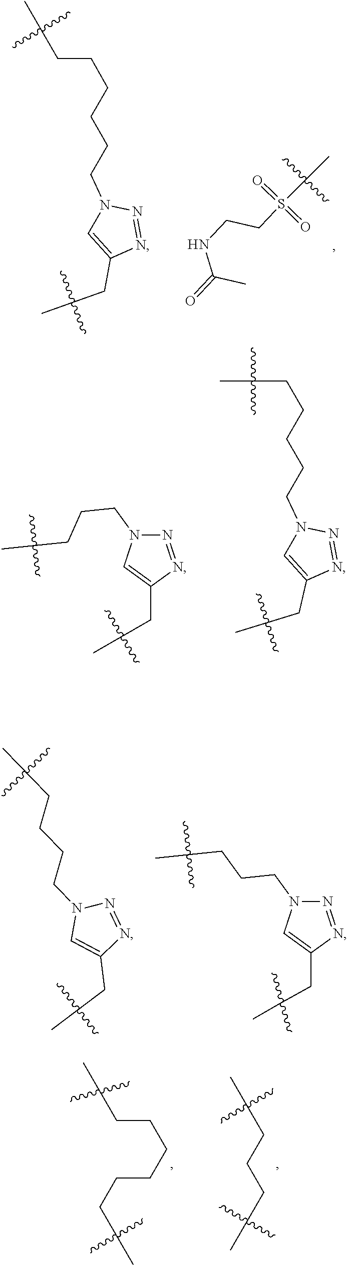

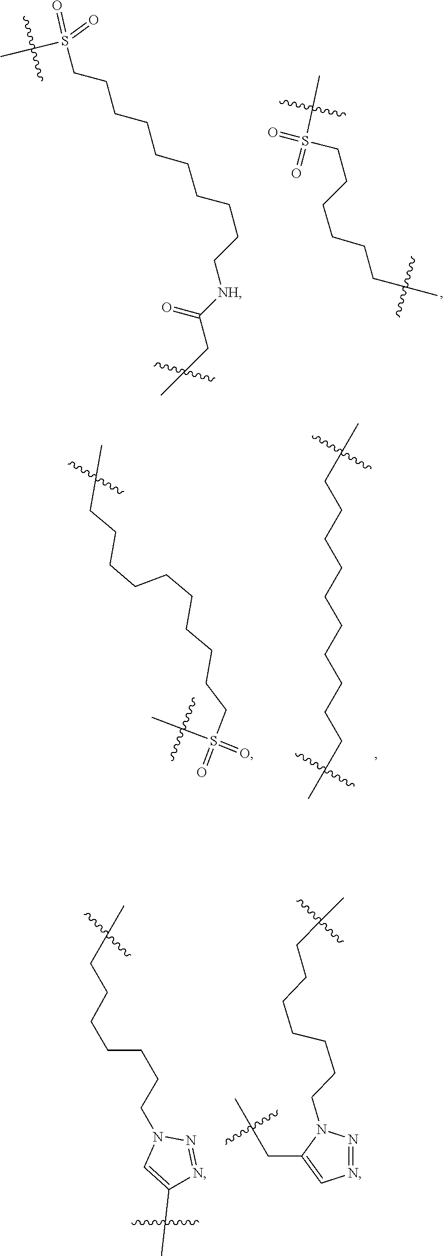 [SMILES: *CC(=O)NCCCCCCCCCCS(*)(=O)=O ; *CCCCCCS(*)(=O)=O ; *CCCCCCCCCCS(*)(=O)=O ; *CCCCCCCCCC* ; *CCCCCCCn1cc(*)nn1 ; *CCCCCCCn1nncc1C*]

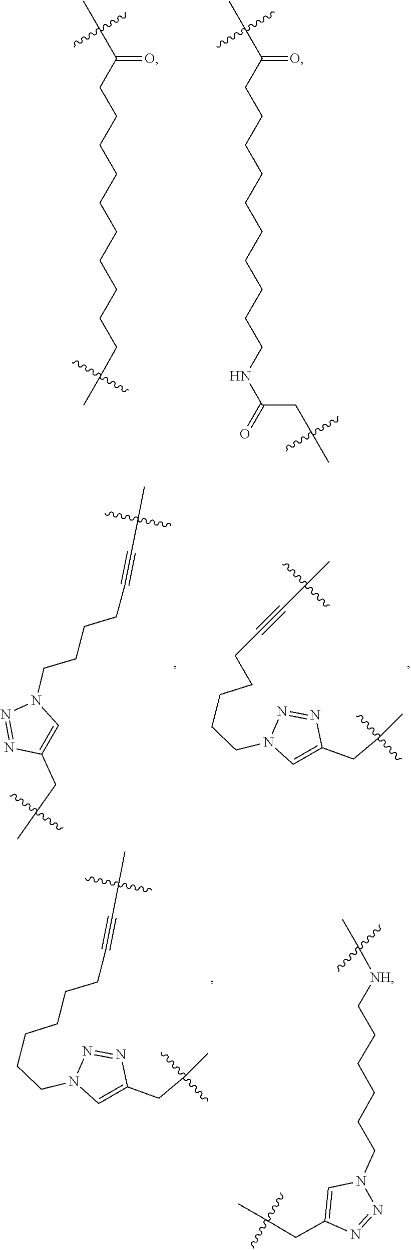 [SMILES: *CCCCCCCCCCC(*)=O ; *CC(=O)NCCCCCCCCCCC(*)=O ; *C#CCCCCn1cc(C*)nn1 ; *C#CCCCCCn1cc(C*)nn1 ; *C#CCCCCCCn1cc(C*)nn1 ; *Cc1cn(CCCCCCN*)nn1]

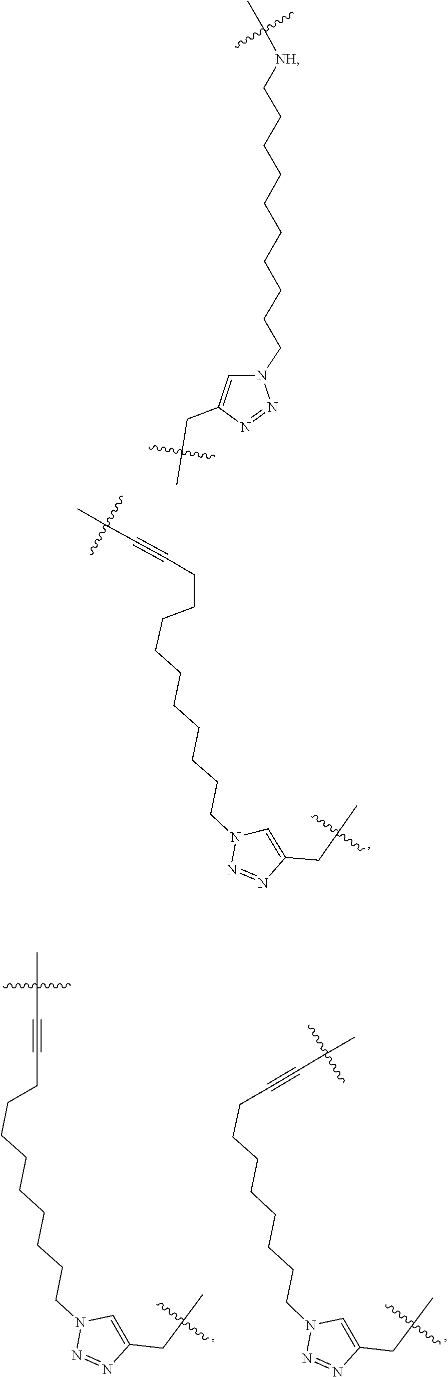 [SMILES: *Cc1cn(CCCCCCCCCCN*)nn1 ; *C#CCCCCCCCCCCn1cc(C*)nn1 ; *C#CCCCCCCCCCn1cc(C*)nn1 ; *C#CCCCCCCCCn1cc(C*)nn1]

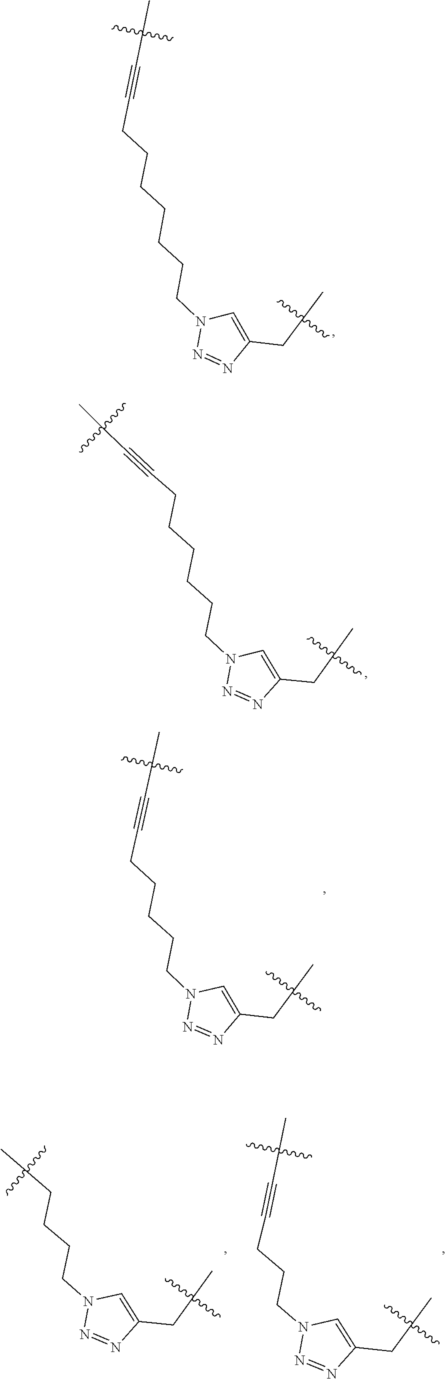 [SMILES: *C#CCCCCCCCn1cc(C*)nn1 ; *C#CCCCCCCn1cc(C*)nn1 ; *C#CCCCCCn1cc(C*)nn1 ; *CCCCn1cc(C*)nn1 ; *C#CCCCn1cc(C*)nn1]

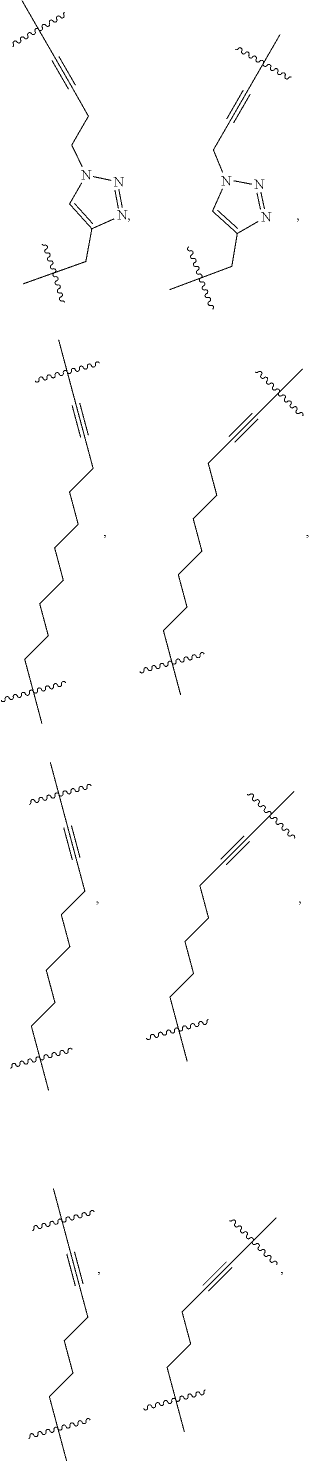

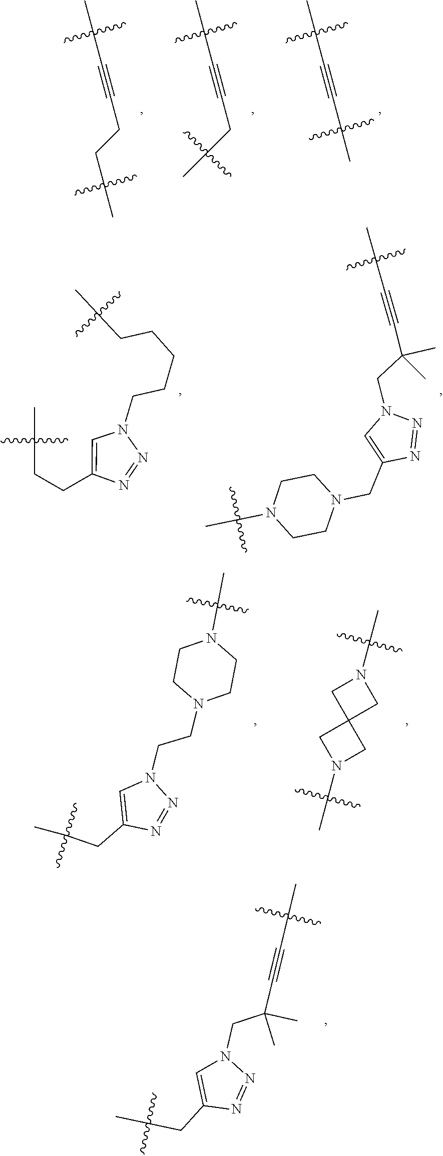

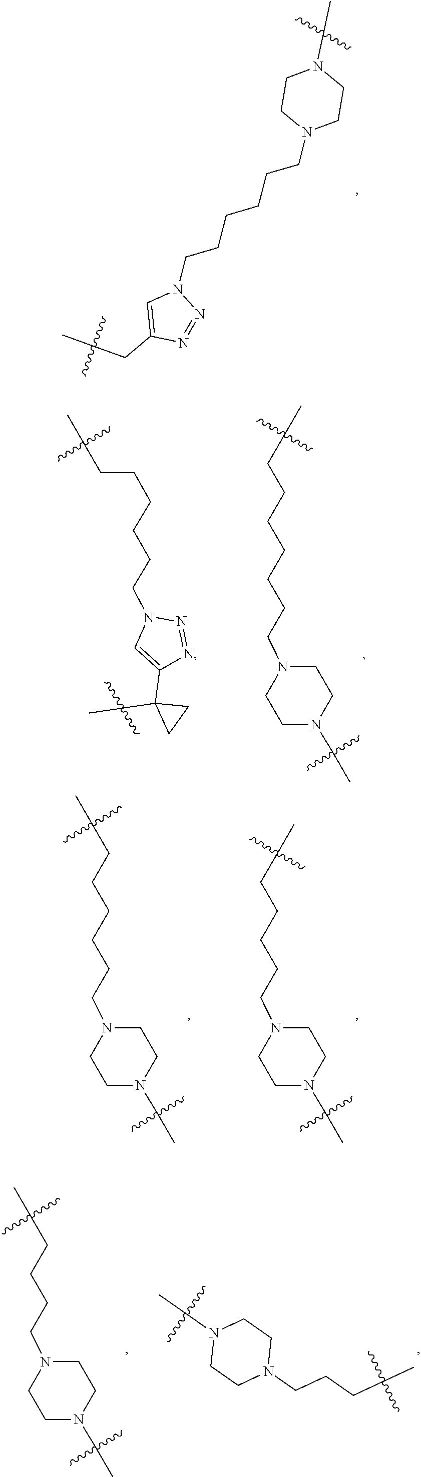

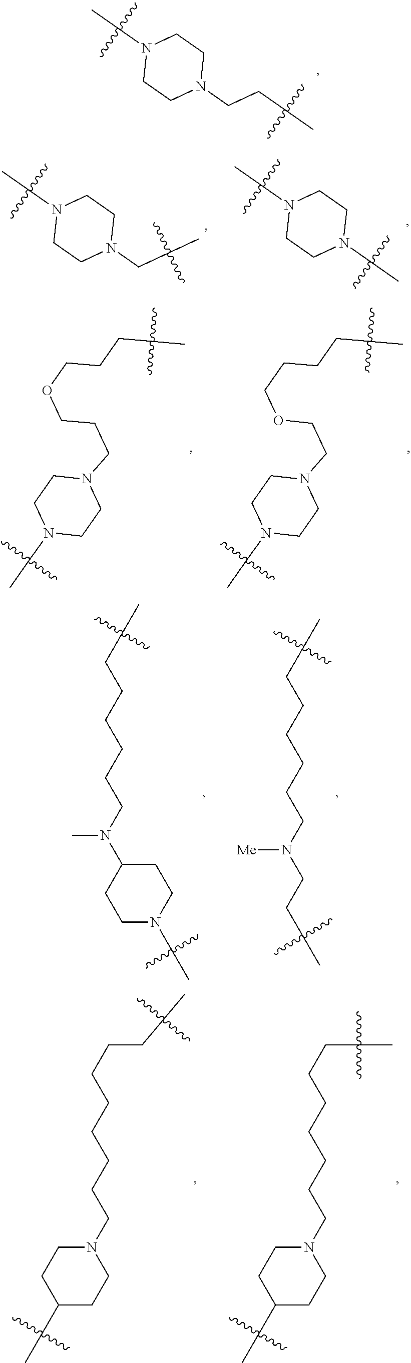

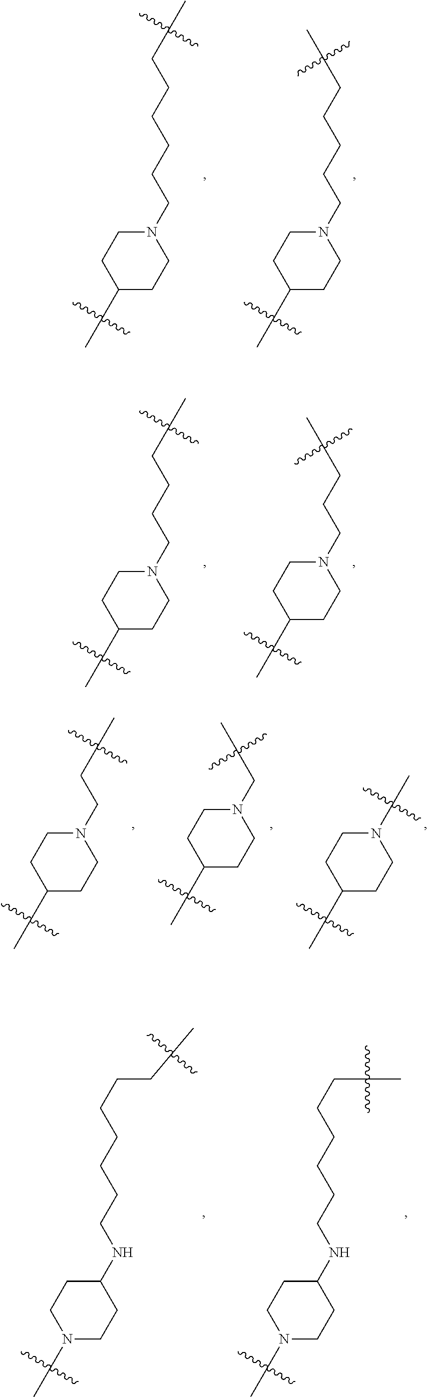

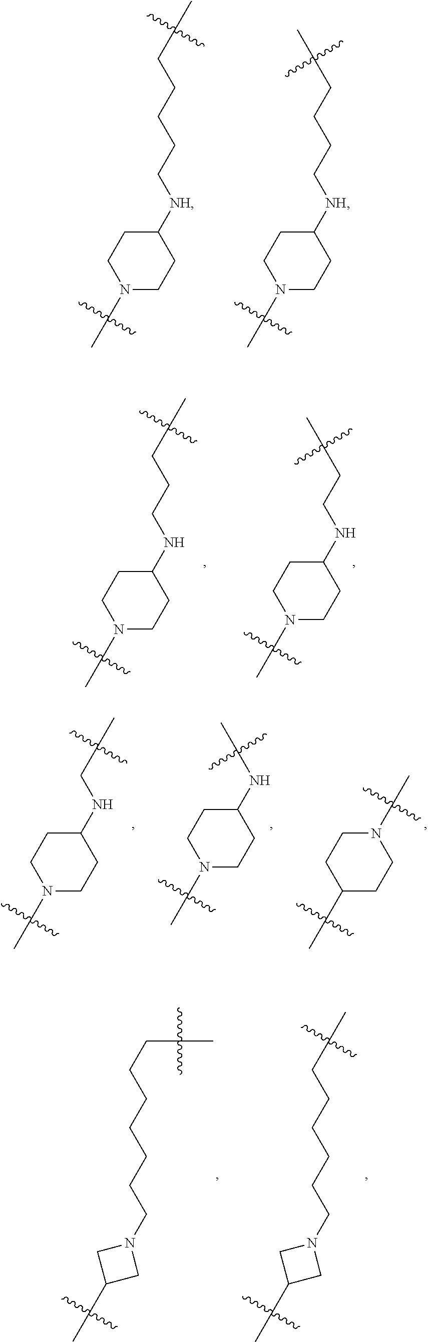

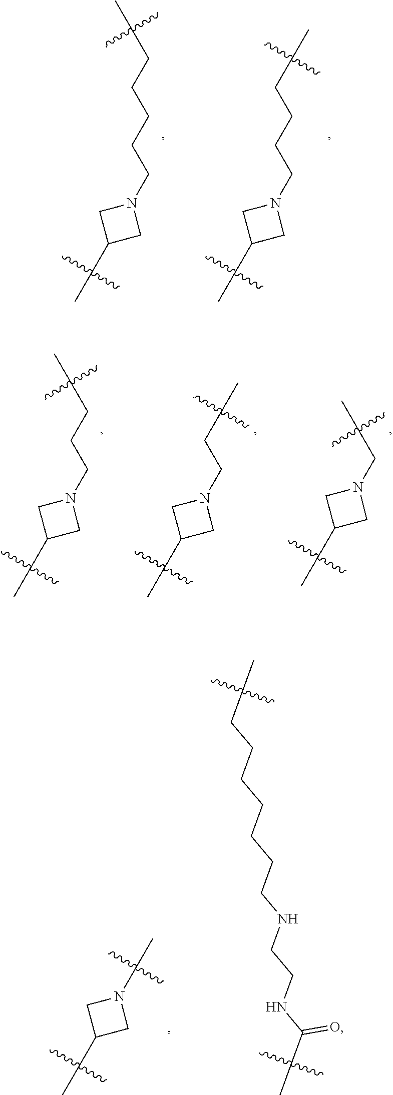

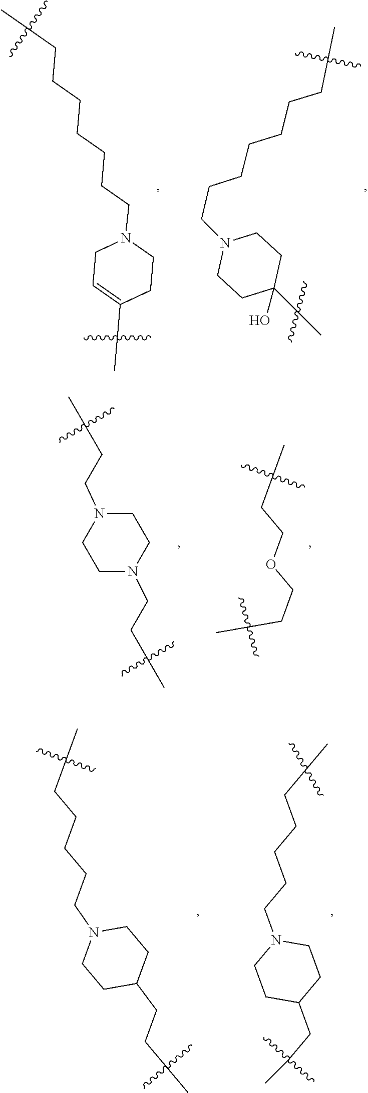 [SMILES: *CCCCCCCN1CC=C(*)CC1 ; *CCCCCCCN1CCC(*)(O)CC1 ; *CCN1CCN(CC*)CC1 ; *CCOCC* ; *CCCCCN1CCC(CC*)CC1 ; *CCCCCN1CCC(C*)CC1]

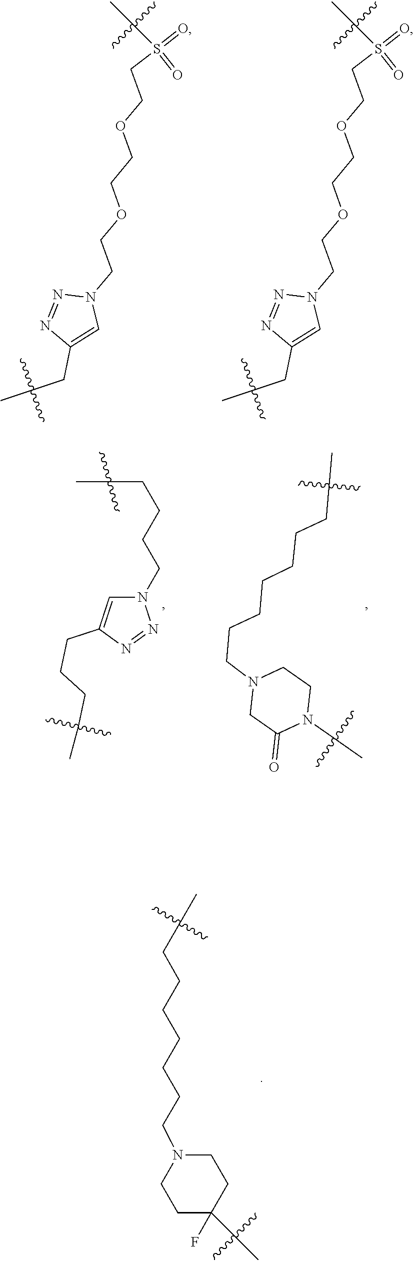 [SMILES: *Cc1cn(CCOCCOCCS(*)(=O)=O)nn1 ; *Cc1cn(CCOCCOCCS(*)(=O)=O)nn1 ; *CCCCn1cc(CCC*)nn1 ; *CCCCCCCN1CCN(*)C(=O)C1 ; *CCCCCCCN1CCC(*)(F)CC1]

In additional embodiments, Linker is selected from the group consisting of:

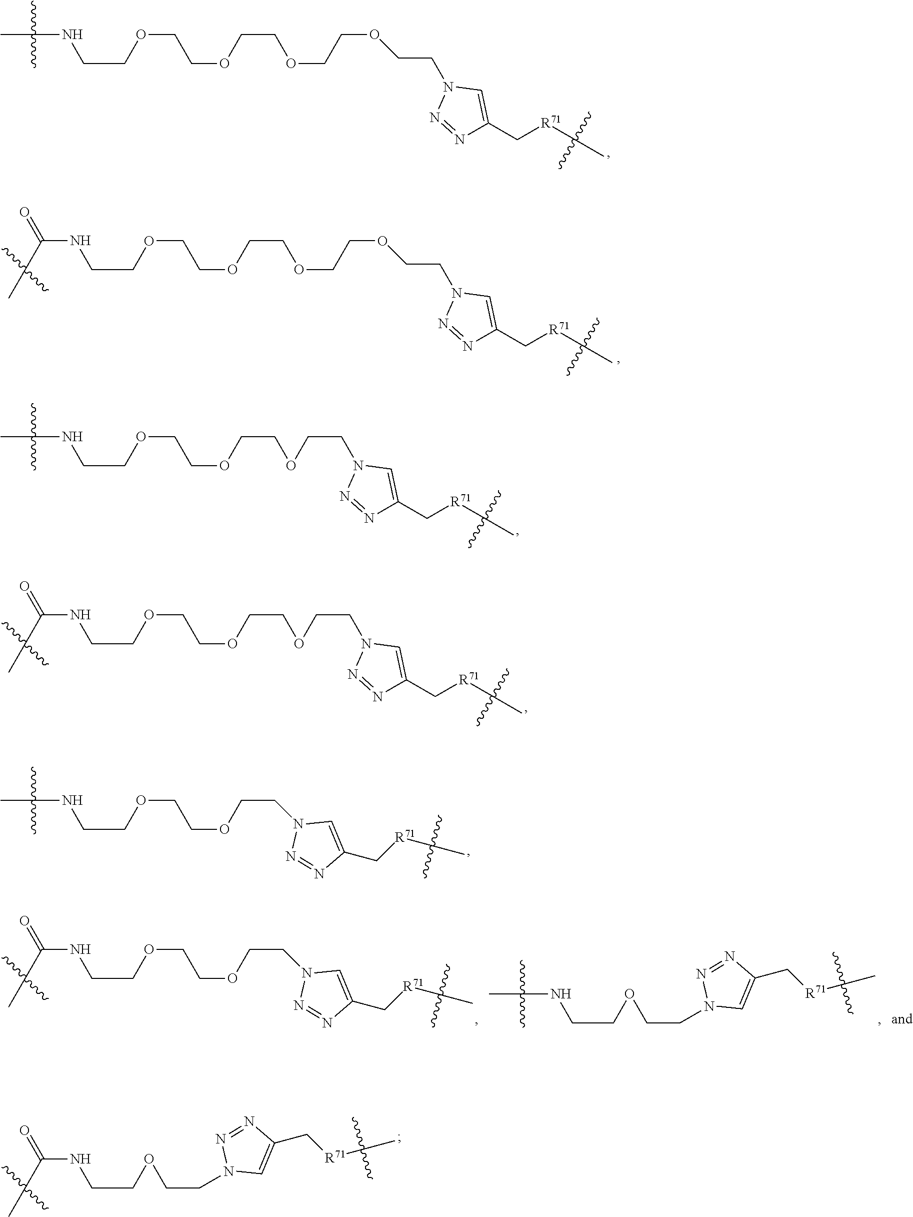

wherein R⁷¹ is —O—, —NH, Nalkyl, alkyl, or -NMe.

In additional embodiments, Linker is selected from the group consisting of:

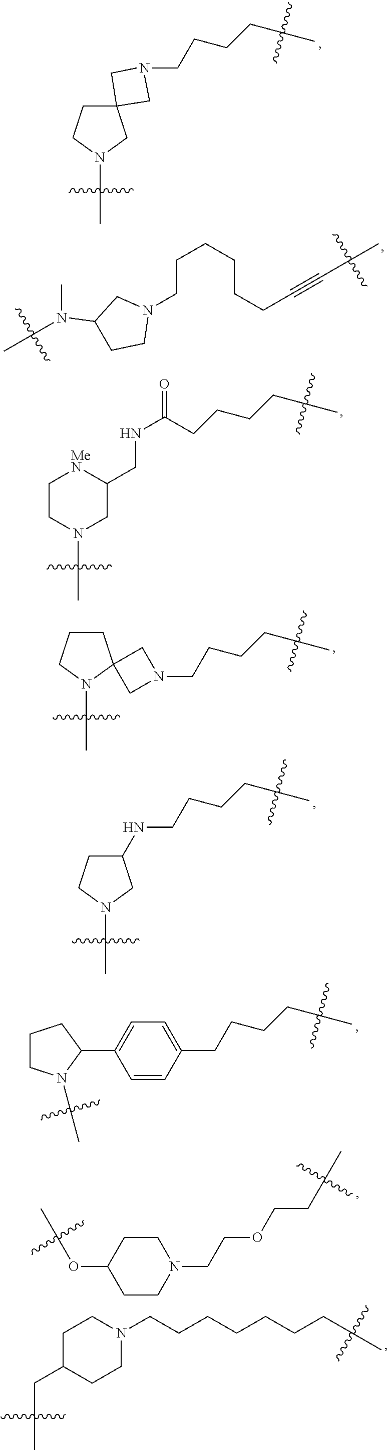

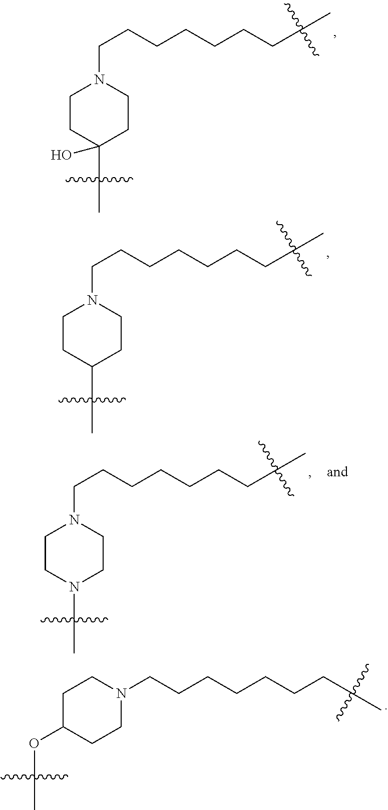 [SMILES: *CCCCCCCN1CCC(*)(O)CC1 ; *CCCCCCCN1CCC(*)CC1 ; *CCCCCCCN1CCN(*)CC1 ; *CCCCCCCN1CCC(O*)CC1]

In additional embodiments, Linker is selected from the group consisting of:

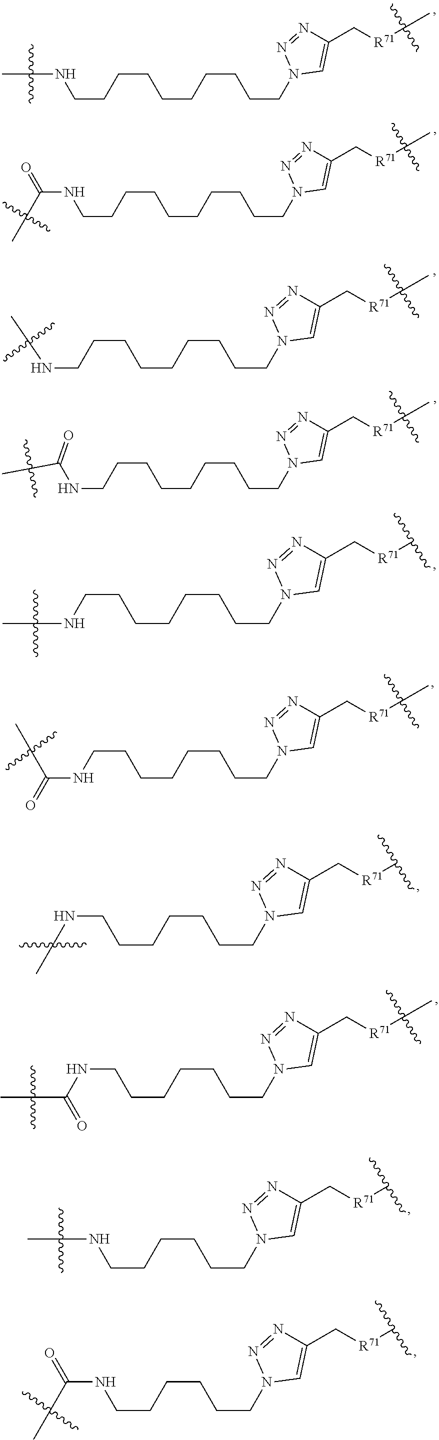

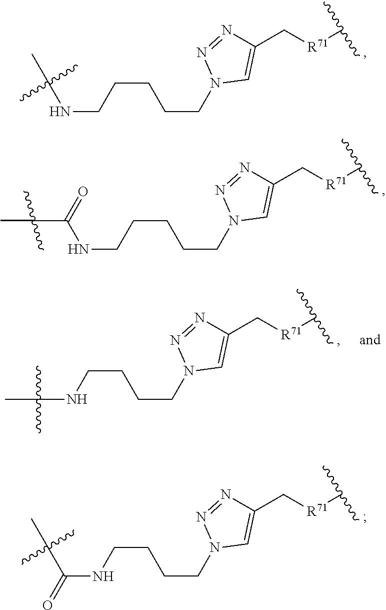

wherein R⁷¹ is —O—, —NH, Nalkyl, alkyl, or -NMe.

In additional embodiments, Linker is selected from the group consisting of:

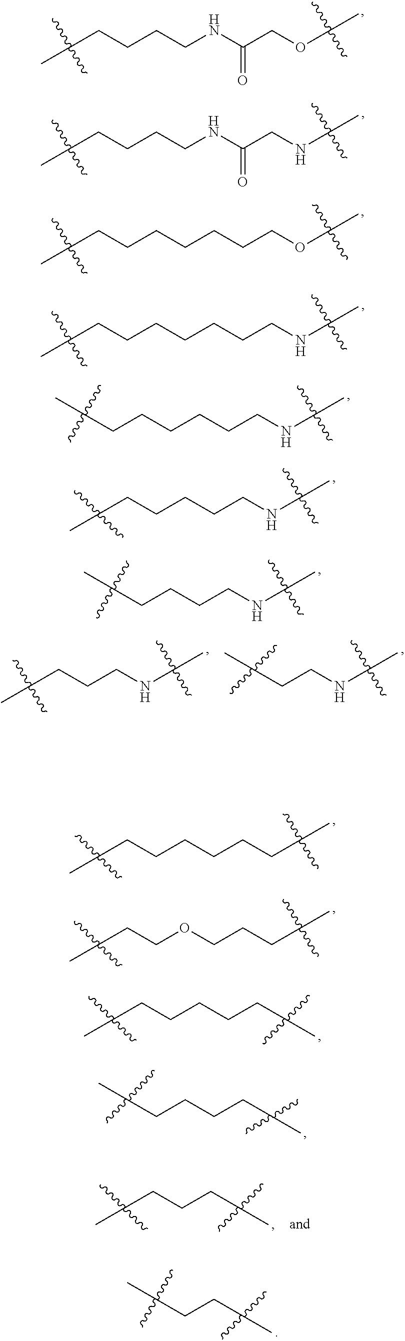

In additional embodiments, Linker is selected from:

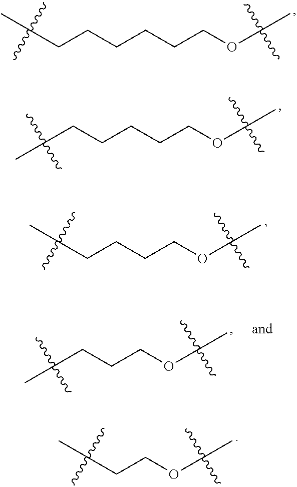 [SMILES: *CCCCCCO* ; *CCCCCO* ; *CCCCO* ; *CCCO* ; *CCO*]

In certain embodiments, Linker is selected from the group consisting of:

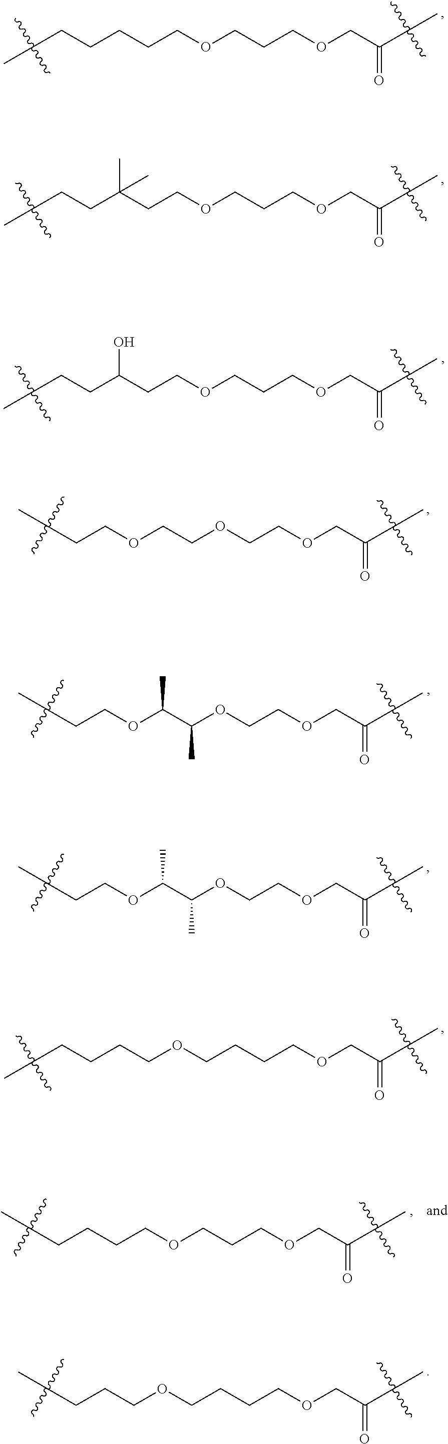

In certain embodiments Linker is selected from the group consisting of:

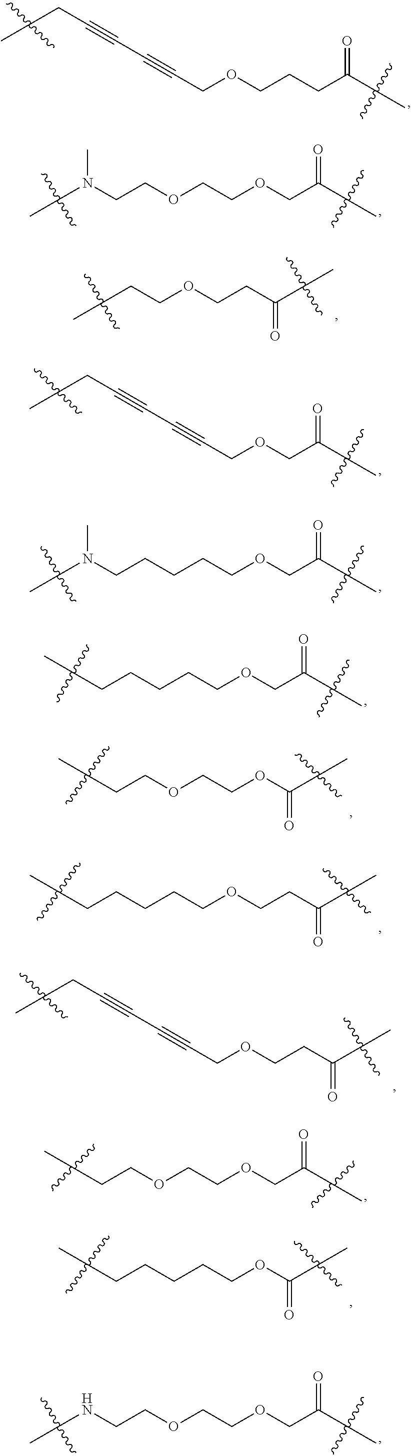

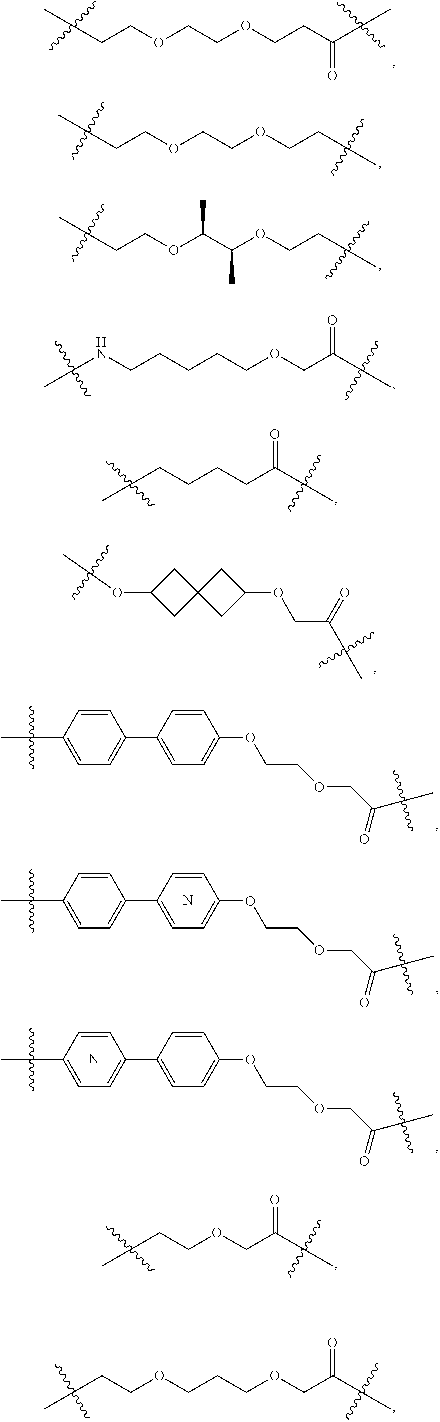

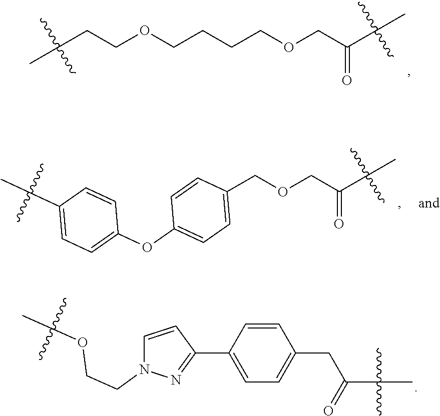 [SMILES: *CCOCCCCOCC(*)=O ; *C(=O)COCc1ccc(Oc2ccc(*)cc2)cc1 ; *OCCn1ccc(-c2ccc(CC(*)=O)cc2)n1]

In the above structures

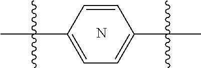

represents

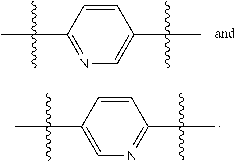

In certain embodiments, Linker can be a 4-24 carbon atom linear chain, wherein one or more the carbon atoms in the linear chain can be replaced or substituted with oxygen, nitrogen, amide, fluorinated carbon, etc., such as the following:

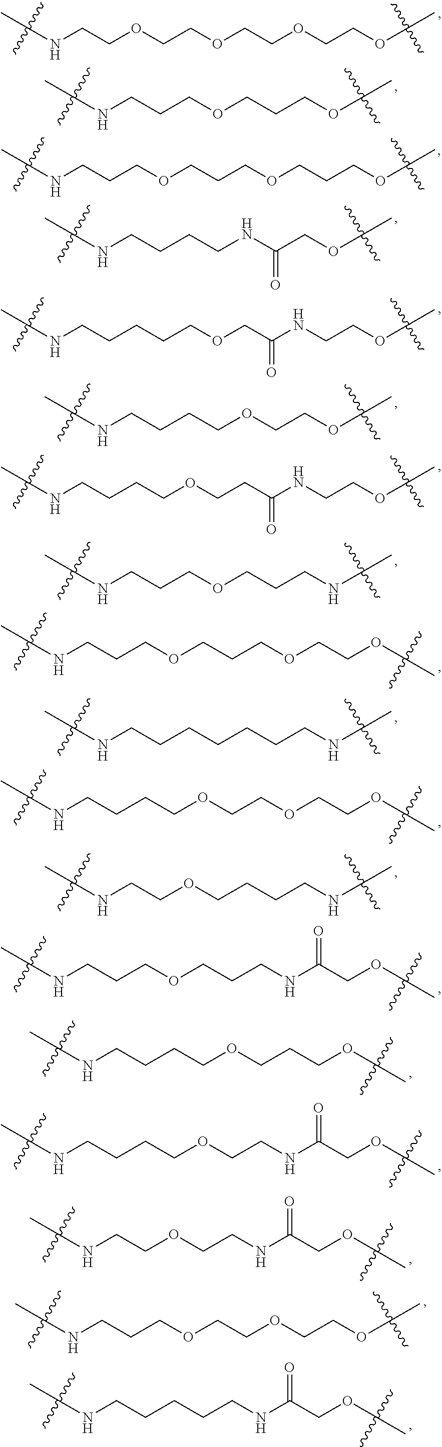

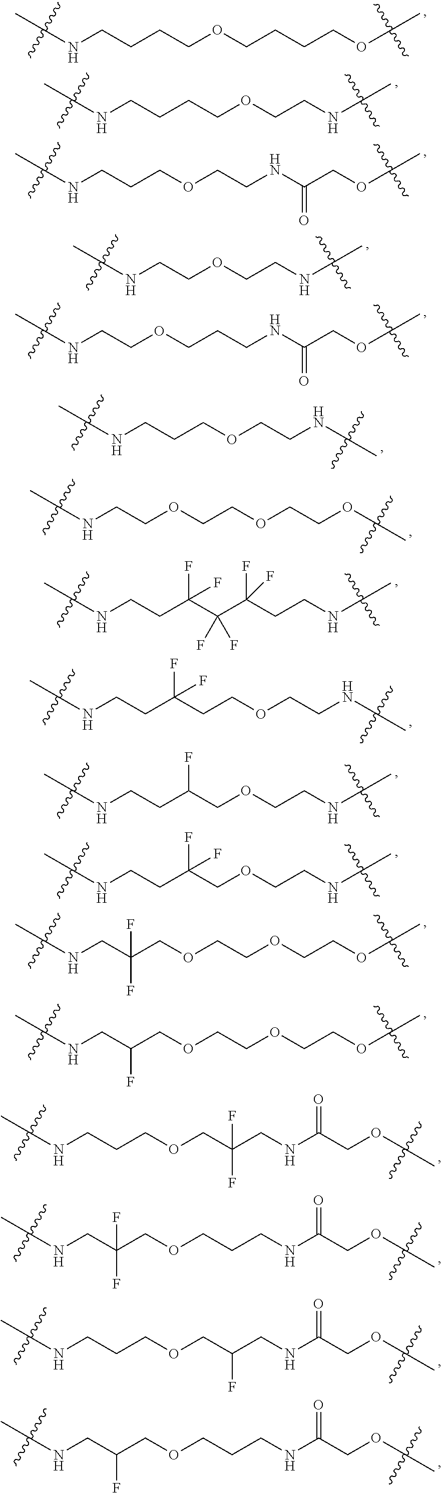

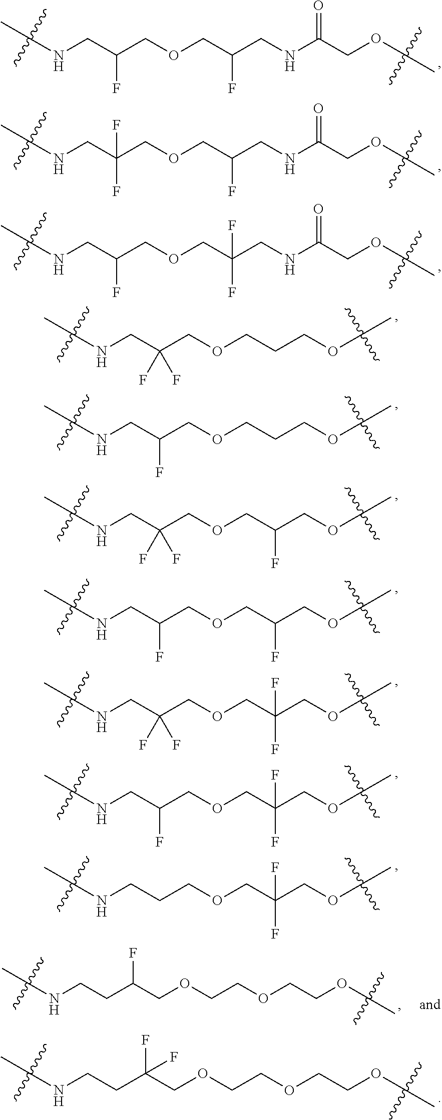

In certain embodiments, Linker may include contiguous, partially contiguous or non-contiguous ethylene glycol unit groups ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units, for example, 1, 2, 3, 4, 6, 6, 7, 8, 9, 10, 11 or 12 ethylene glycol units.

In certain embodiments, Linker may have 1, 2, 3, 4, 5, 6, 7, 8, 9, 10, 11, 12, 13, 14, or fluorine substituents. In another embodiment Linker is perfluorinated. In yet another embodiment Linker is a partially or fully fluorinated poly ether. Nonlimiting examples of fluorinated Linker moieties include:

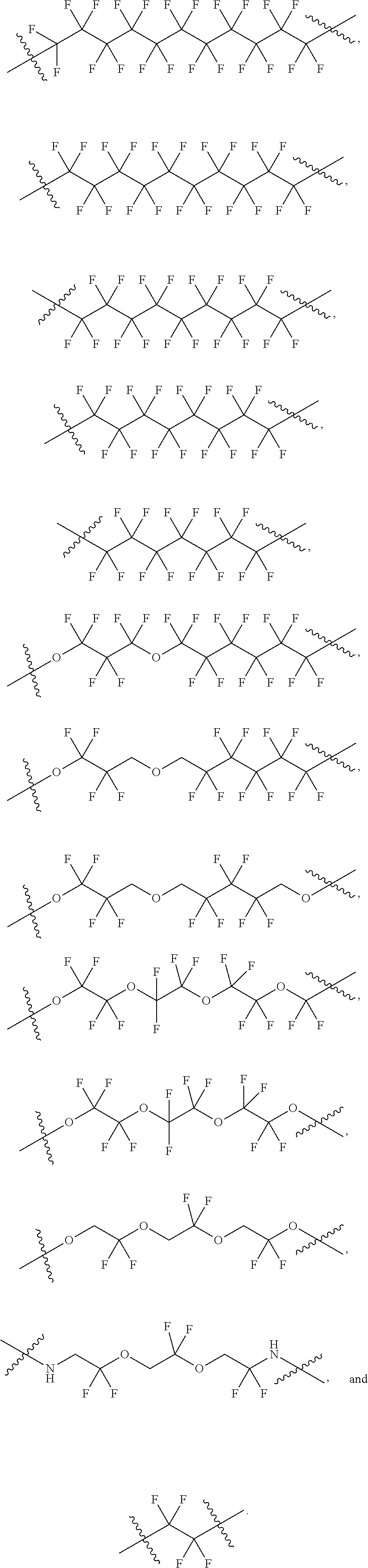

In certain embodiments, where the Target Ligand binds more than one protein (i.e., is not completely selective), selectivity may in some cases be enhanced by varying Linker length where the ligand binds some of its targets in different binding pockets, e.g., deeper or shallower binding pockets than others. Therefore, the length can be adjusted as desired.

In another embodiment, -Linker-Targeting Ligand is -Tail, wherein Tail is a monovalent group. In one embodiment, Tail is covalently attached to at least one Degron and is not attached to a Targeting Ligand. In another embodiment, -Linker-Targeting Ligand is -(Linker)^{C}, wherein -(Linker)^{C} is covalently attached to a Targeting Ligand and one or more additional Targeting Ligands and/or Degrons.

In certain embodiment, Tail is of formula

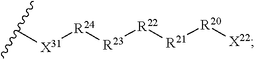

wherein all variables are defined as above.

In certain embodiment, Tail is a moiety selected from Formula TI, Formula TII, Formula TIII, Formula TIV, Formula TV, Formula TVI, and Formula TVII:

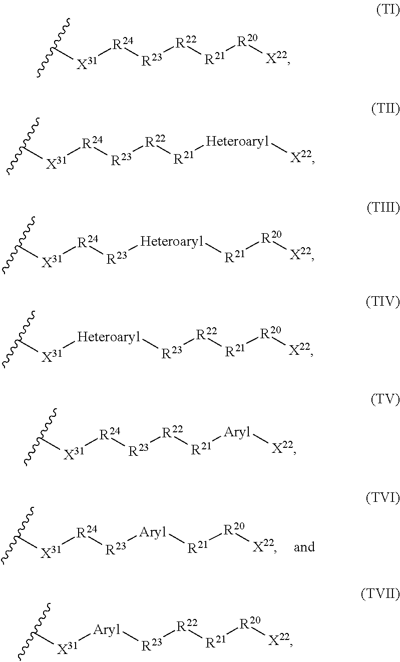

wherein all variables are defined as above.

In additional embodiments, Tail is a moiety selected from Formula TVIII, TIX, and TX:

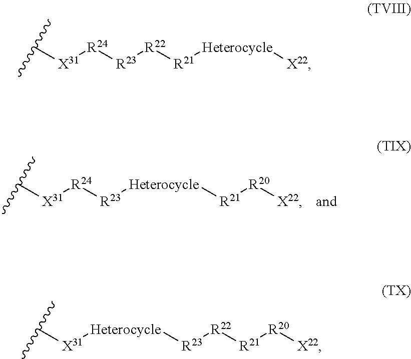

wherein all variables are defined as above. In other embodiments of TVIII, TIX and TX, a carbocyclic ring is used in place of the heterocycle.

The following are non-limiting examples of Tail moieties that can be used in this invention. Based on this elaboration, those of skill in the art will understand how to use the full breadth of Tail moieties that will accomplish the goal of the invention.

As certain non-limiting examples, Formula TI, Formula TII, Formula TIII, Formula TIV, Formula TV, Formula TVI, or Formula TVII include:

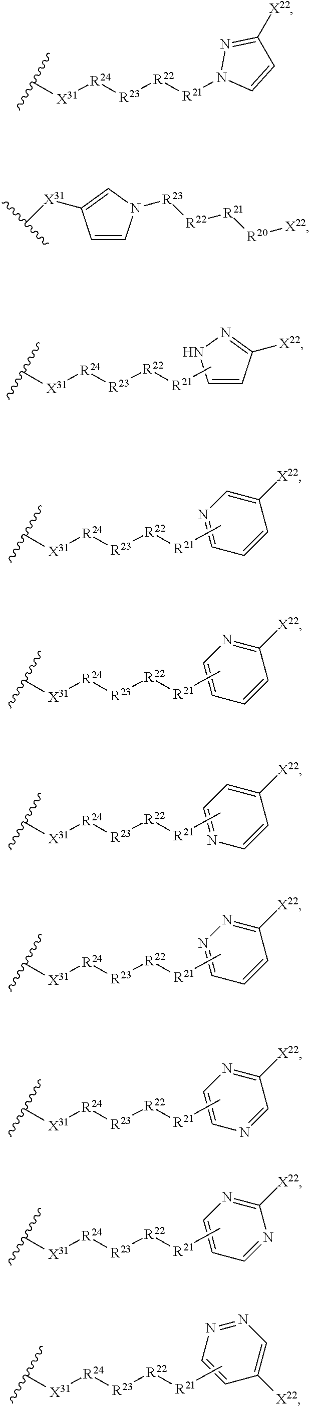

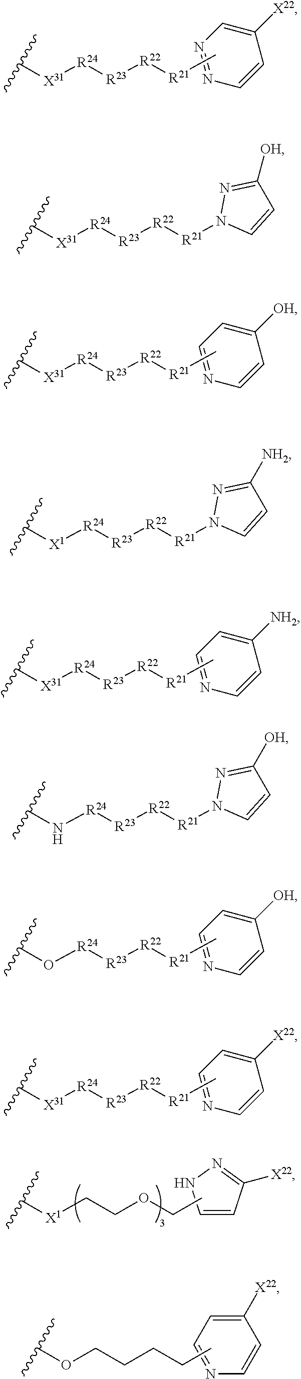

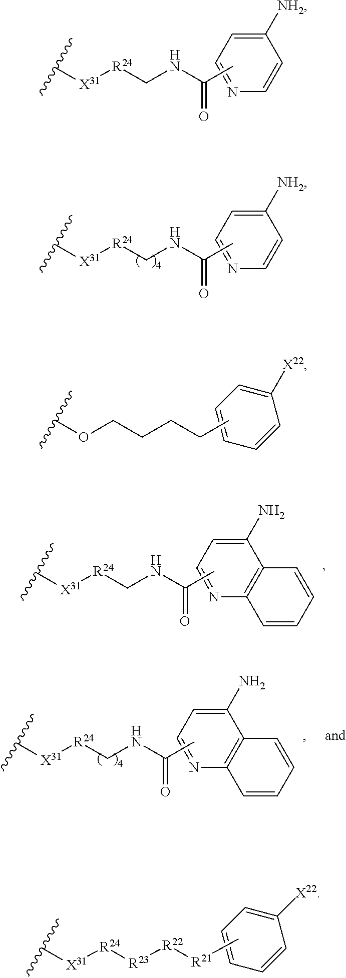

In additional embodiments Tail can be selected from the group consisting of:

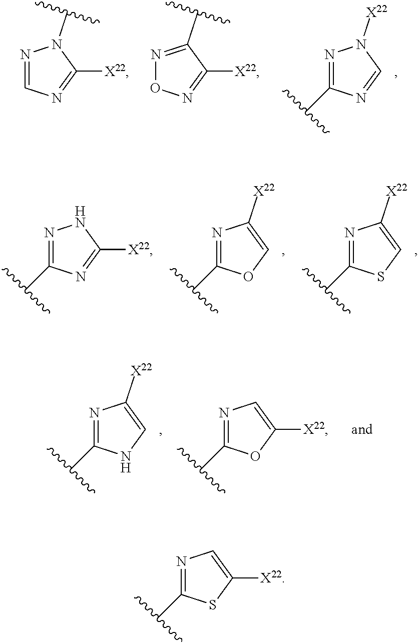

In additional embodiments Tail is selected from the group consisting of:

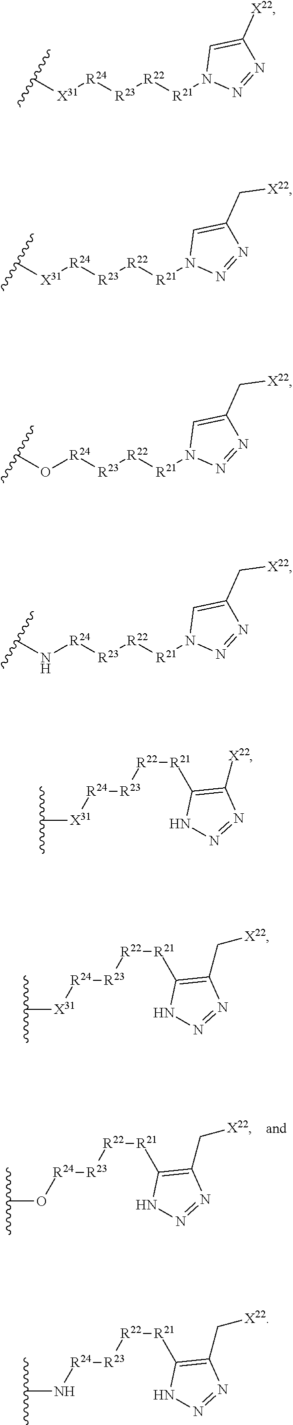

In additional embodiments, Tail is an optionally substituted ethylene glycol having at least 1, at least 2, at least 3, at least 4, at least 5, at least 6, at least 7, at least 8, at least 9, at least 10, ethylene glycol units, or optionally substituted alkyl groups interspersed with optionally substituted, O, N, S, P or Si atoms. In certain embodiments, Tail is flanked, substituted, or interspersed with an aryl, phenyl, benzyl, alkyl, alkylene, or heterocycle group. In certain embodiments, Tail may be asymmetric or symmetrical. In some embodiments, Tail is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units. In any of the embodiments of the compounds described herein, Tail group may be any suitable moiety as described herein.

In additional embodiments, Tail is selected from the group consisting of:

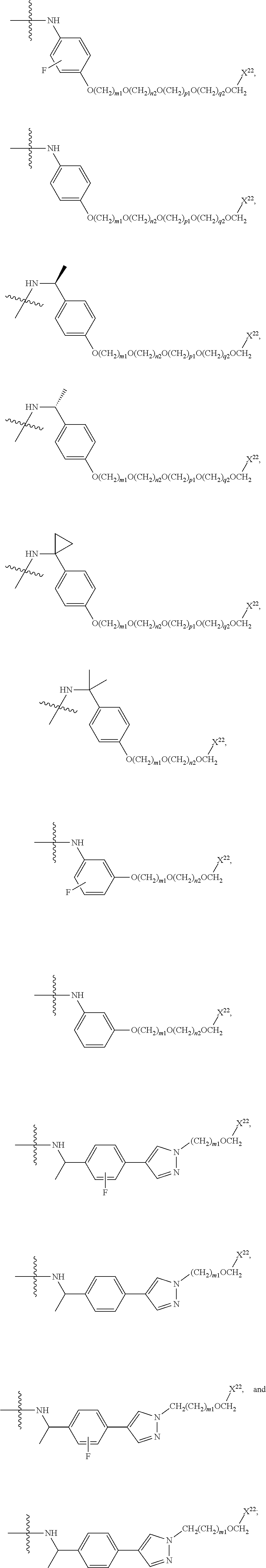

wherein m1, n2, p 1, and q2 are independently 1, 2, 3, 4, or 5.

In additional embodiments, Tail is selected from the group consisting of:

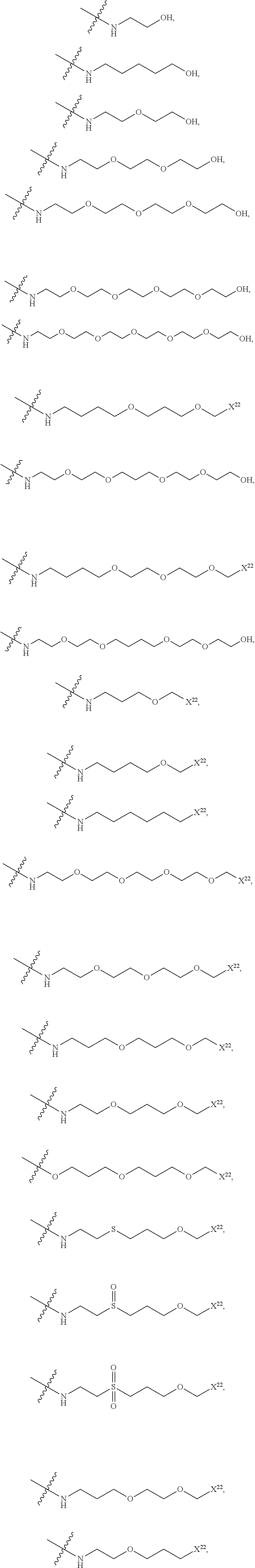

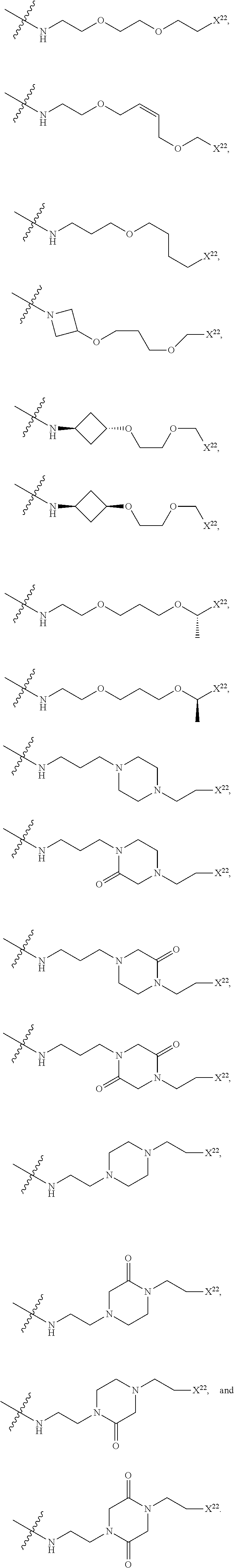

In additional embodiments, Tail is selected from the group consisting of:

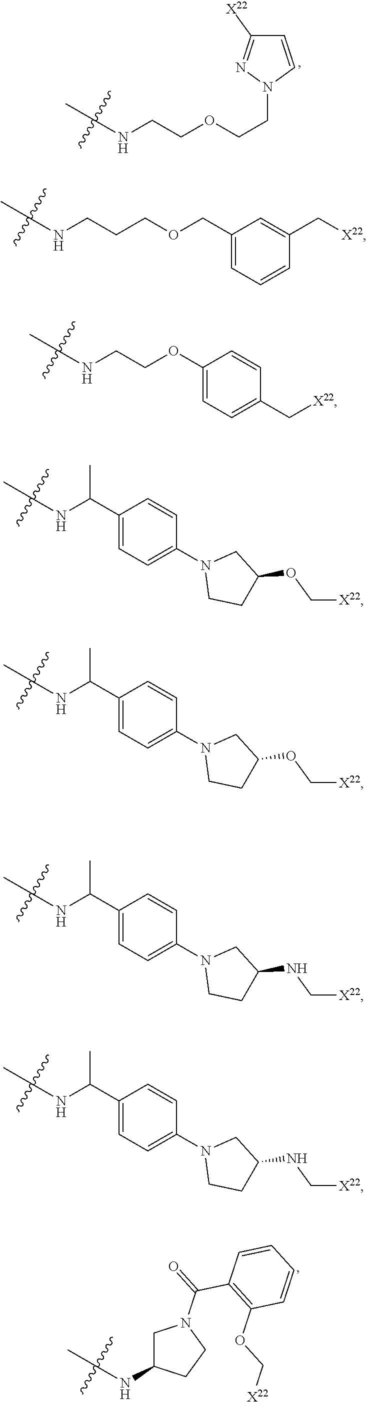

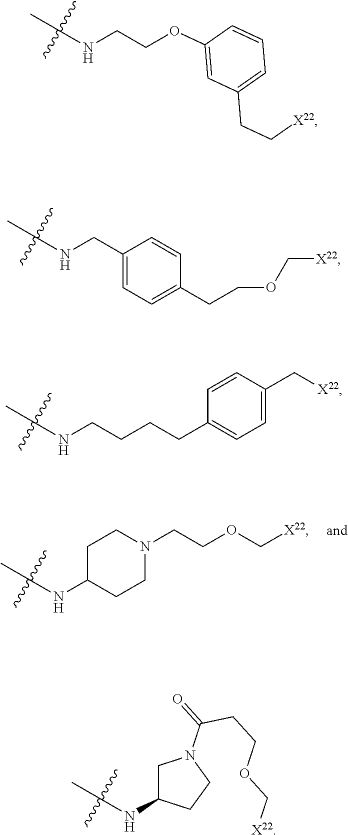

In additional embodiments, Tail is selected from the group consisting of:

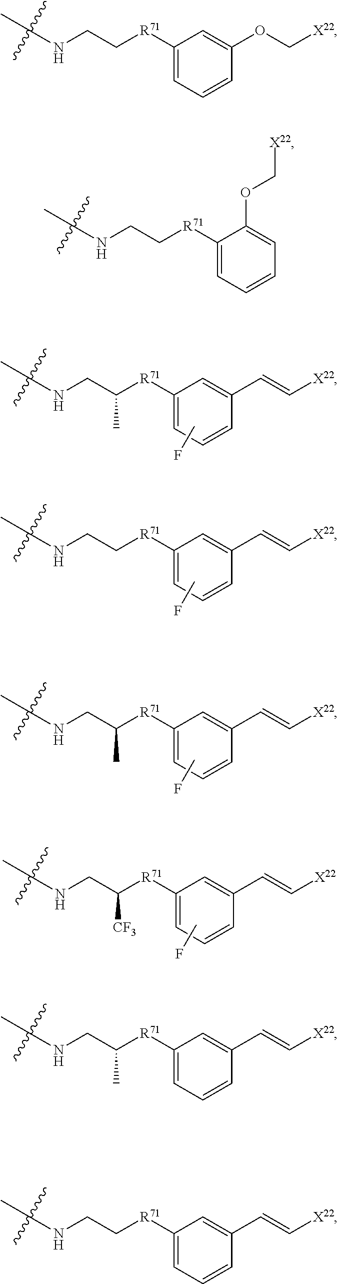

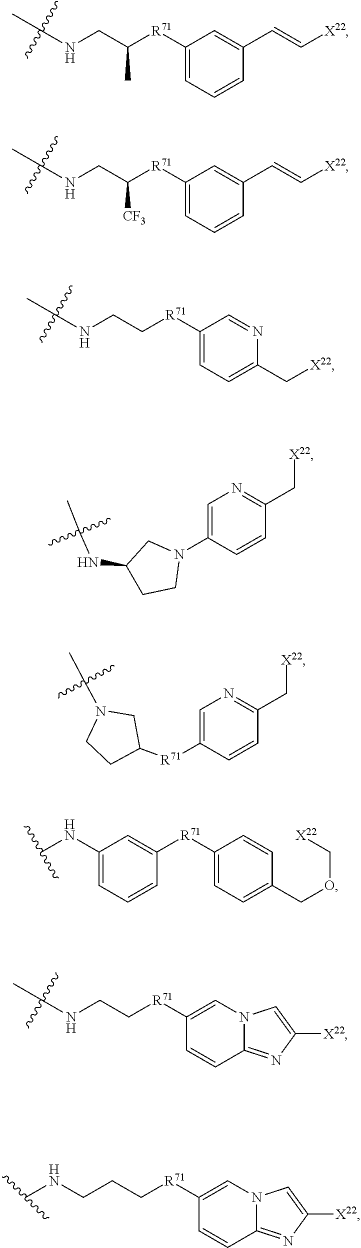

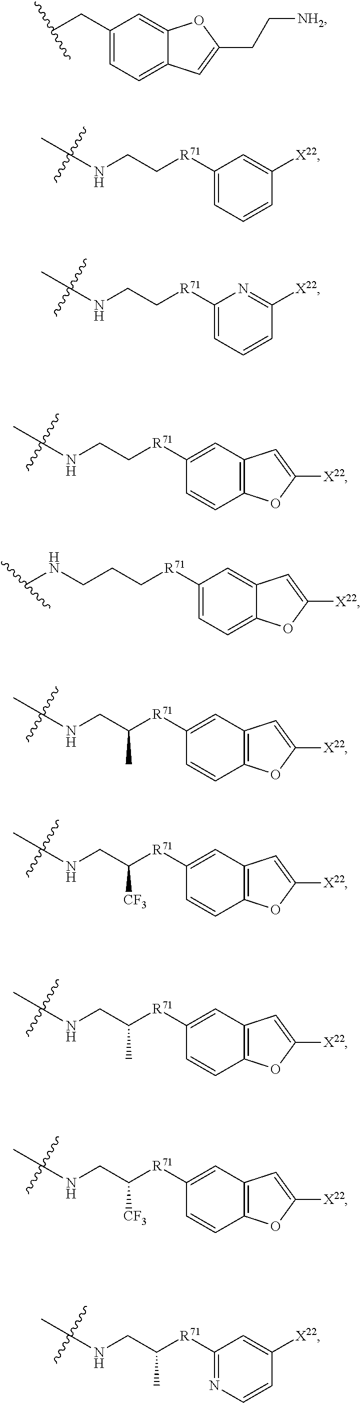

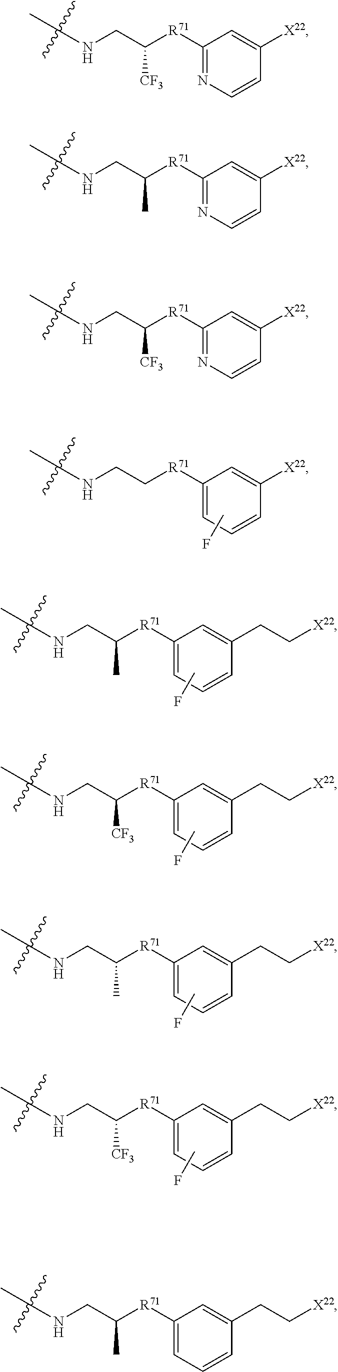

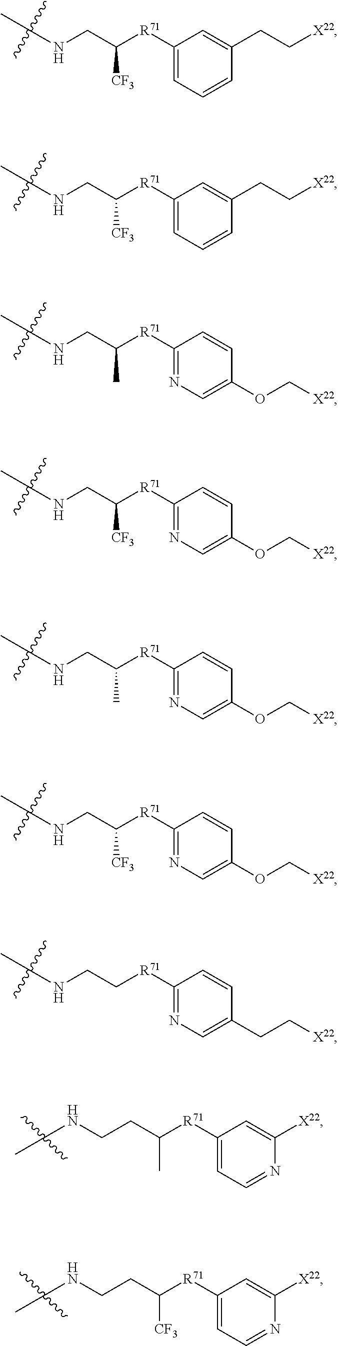

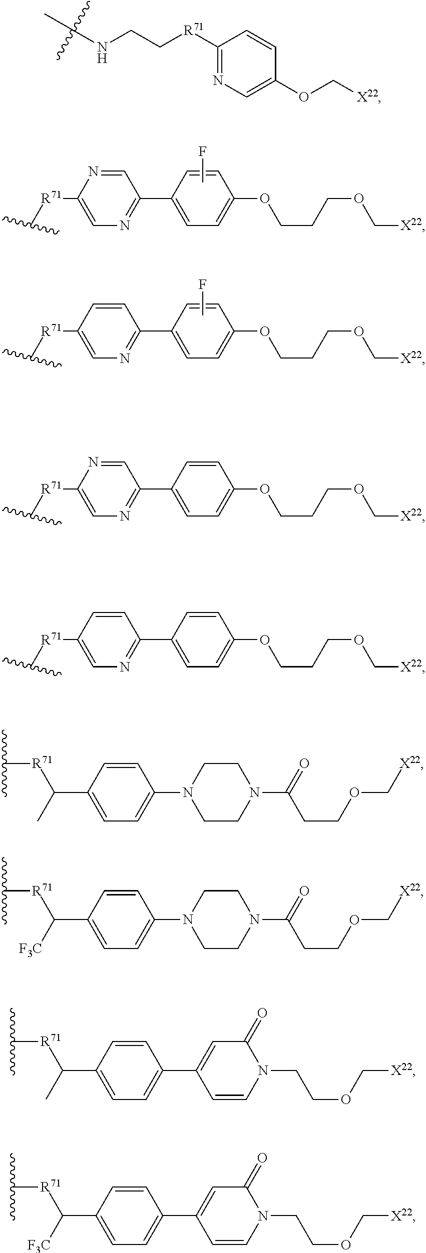

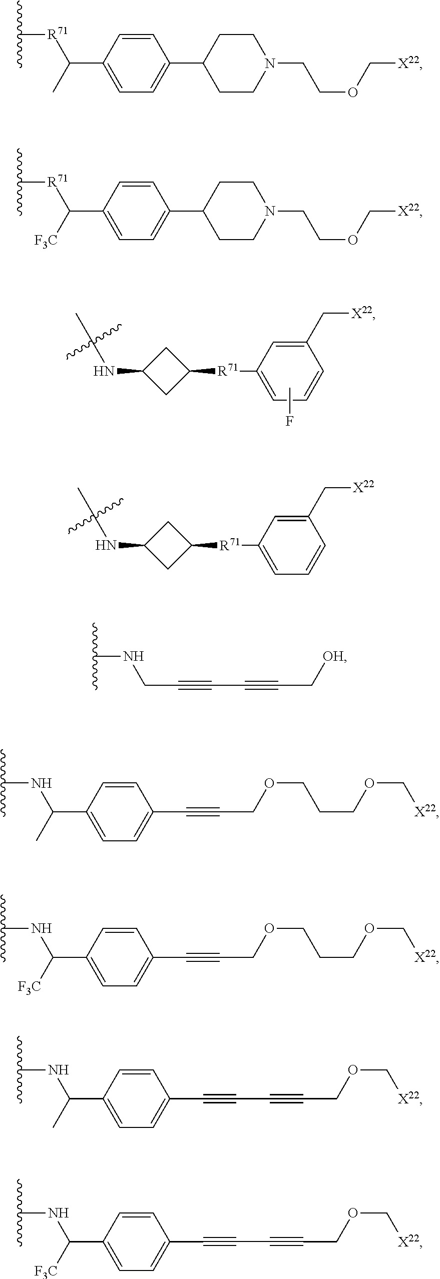

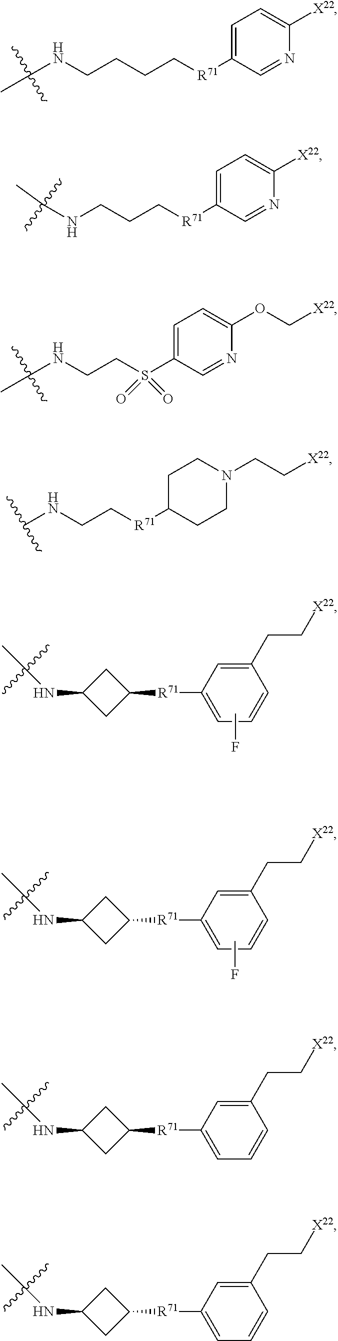

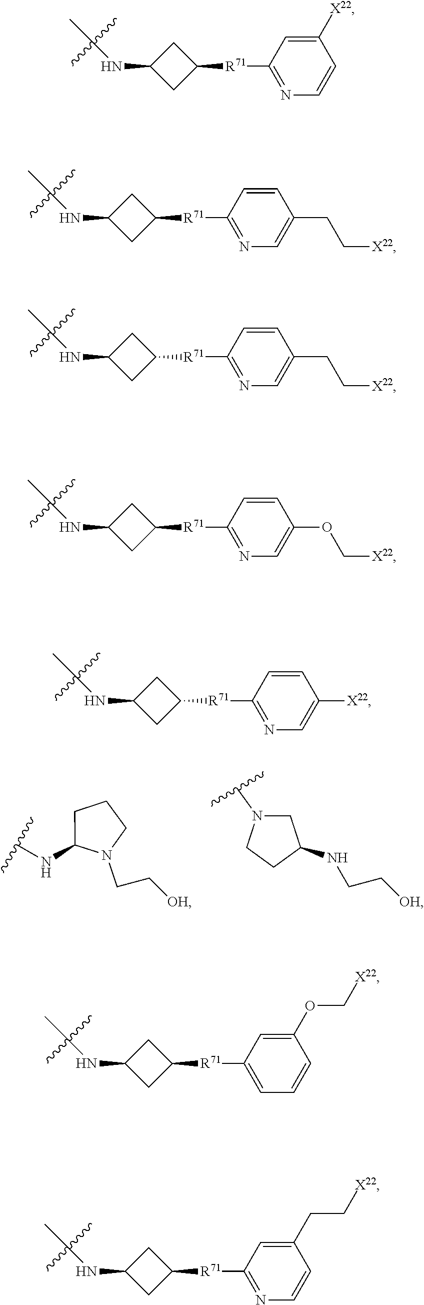

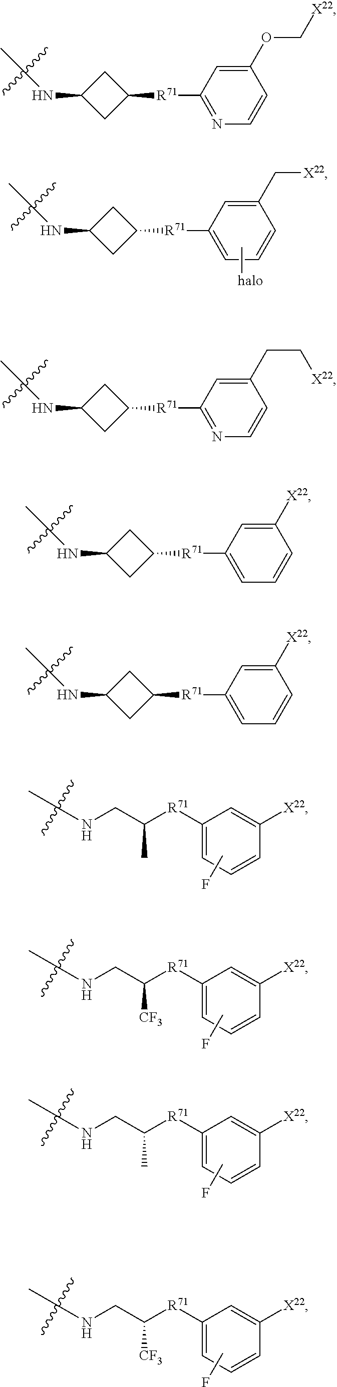

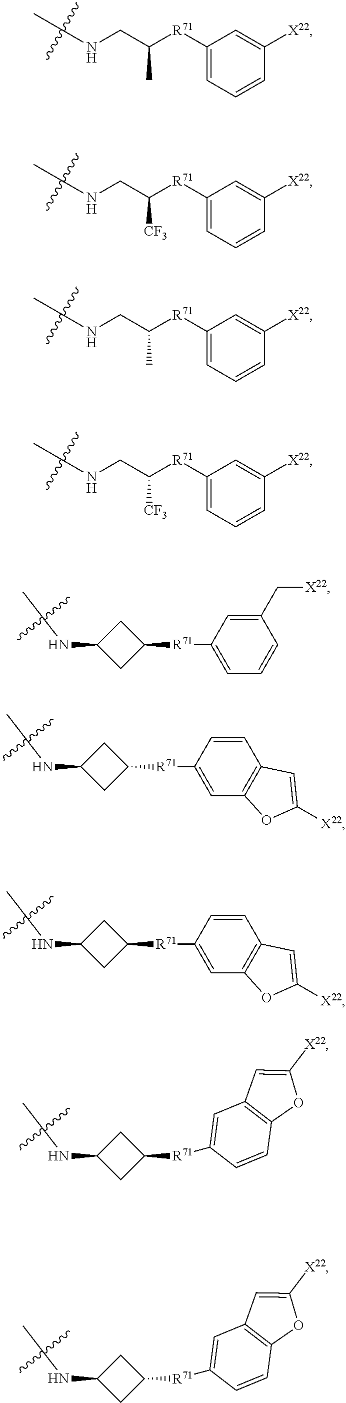

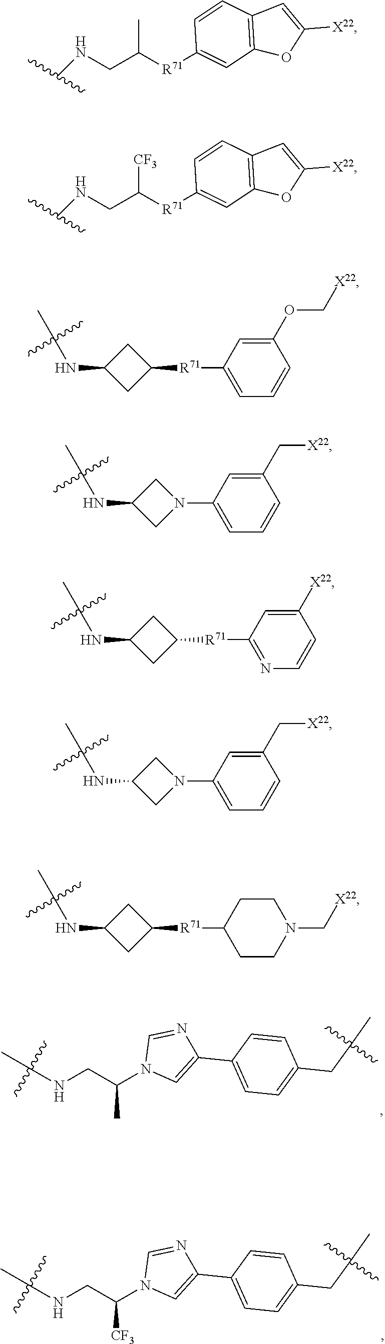

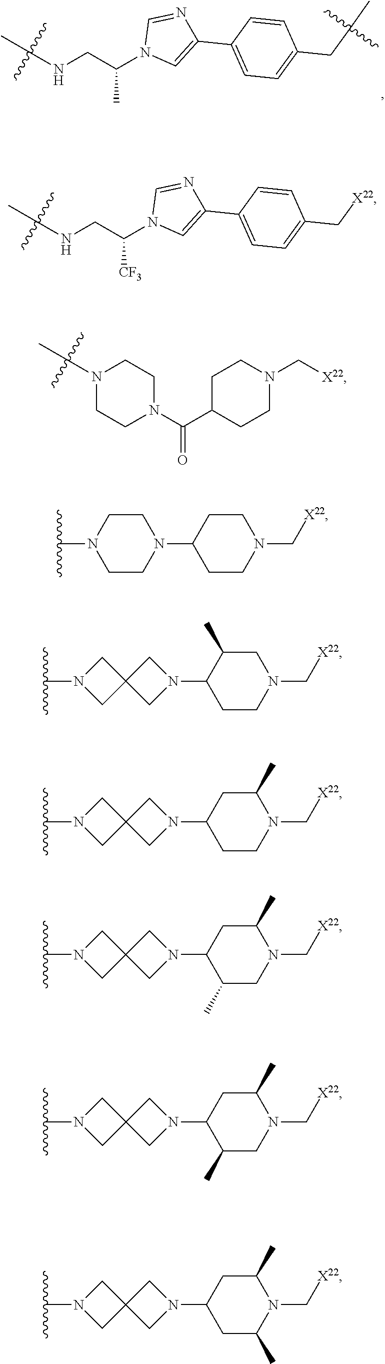

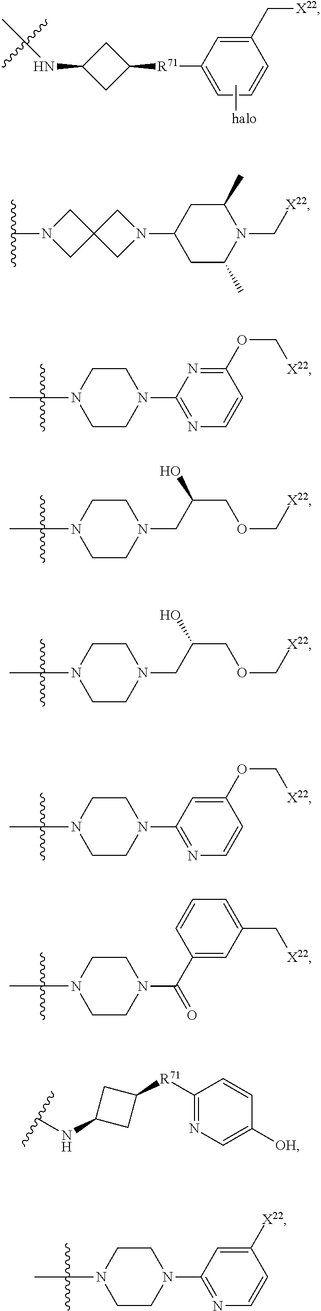

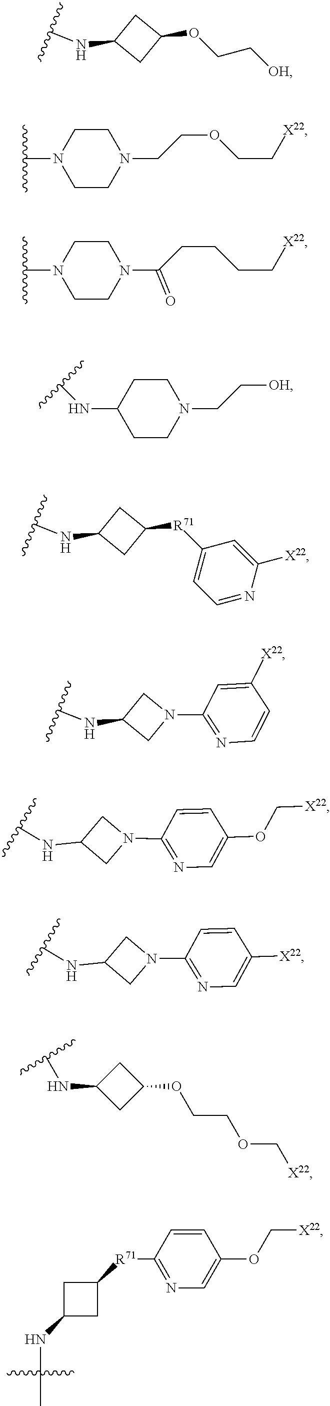

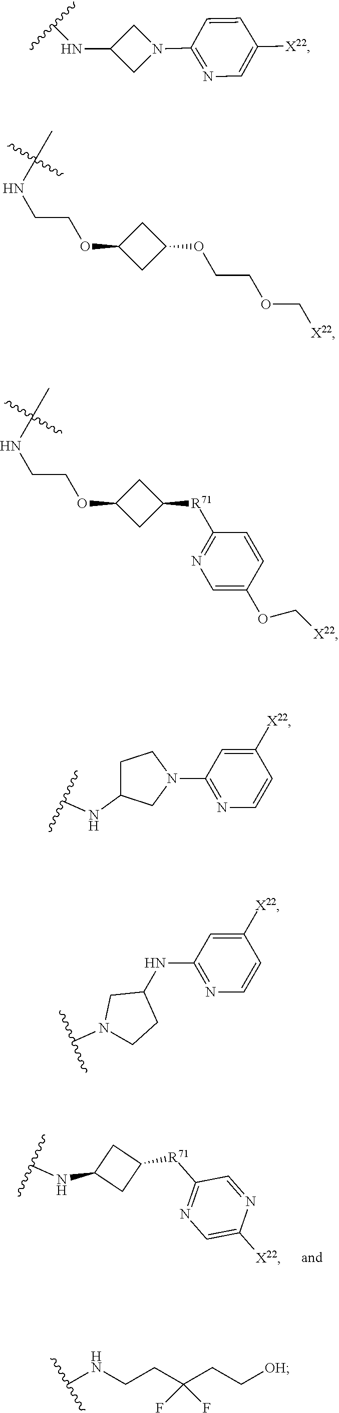

wherein R⁷¹ is —O—, —NH, Nalkyl, alkyl, or -NMe.

In additional embodiments, Tail is selected from the group consisting of:

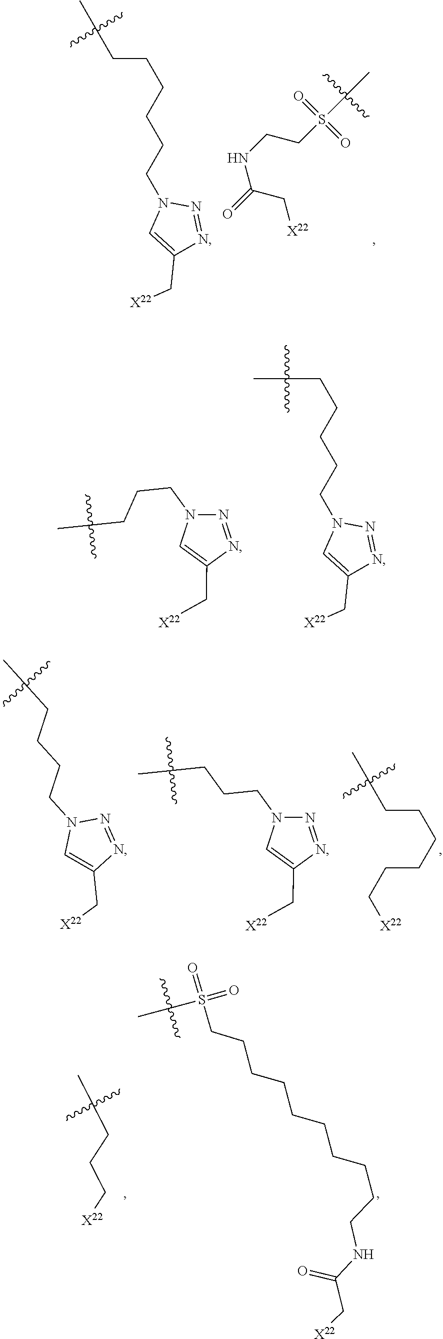

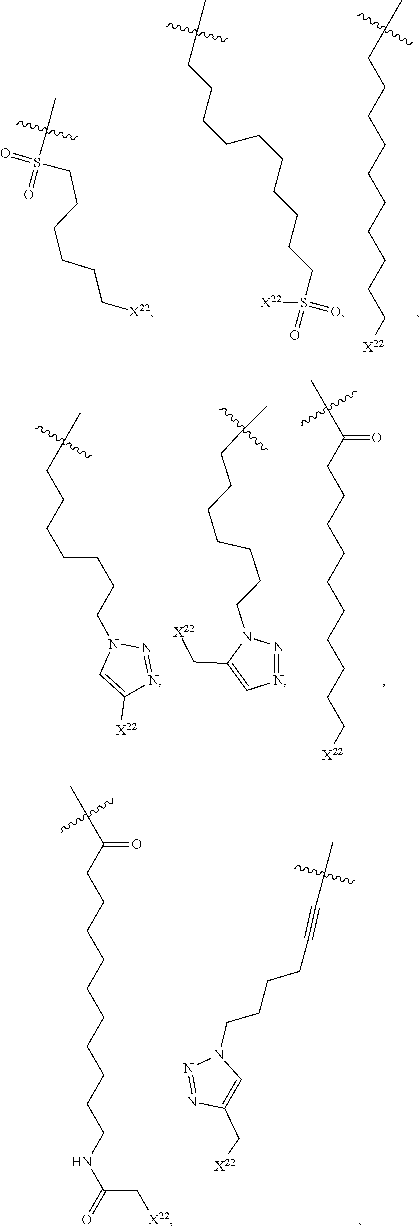

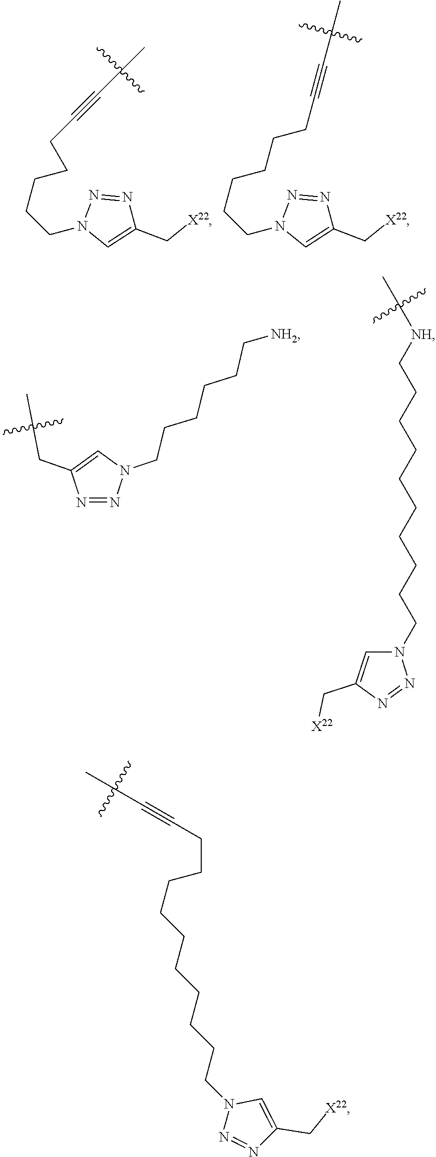

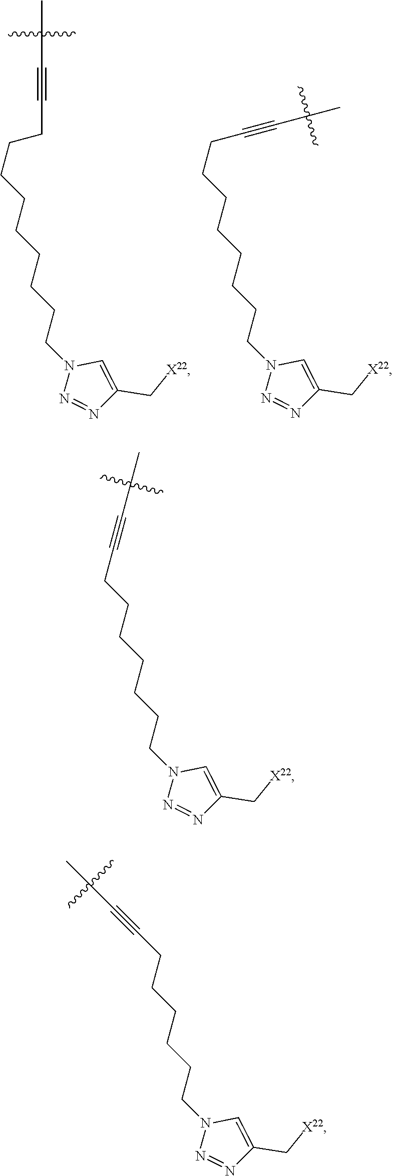

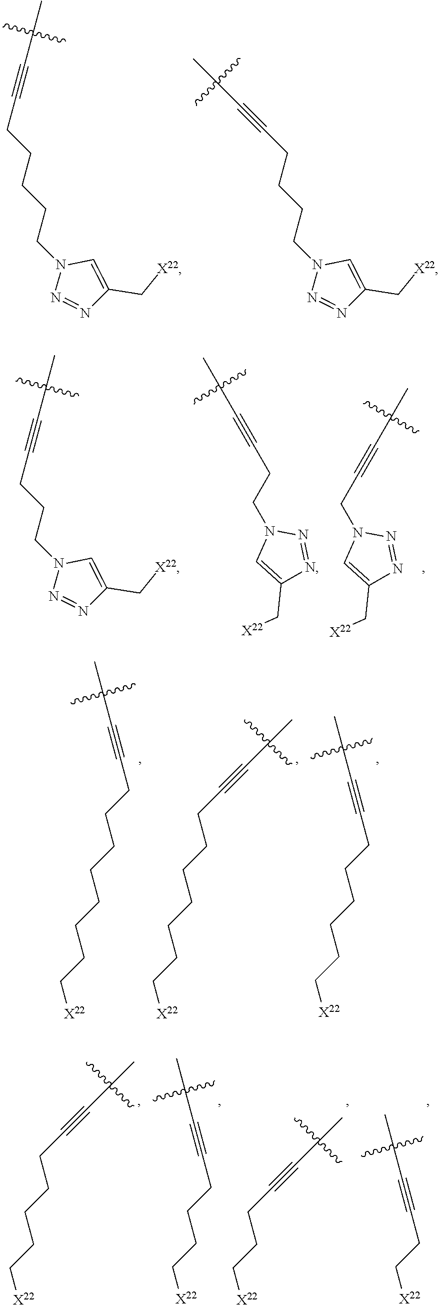

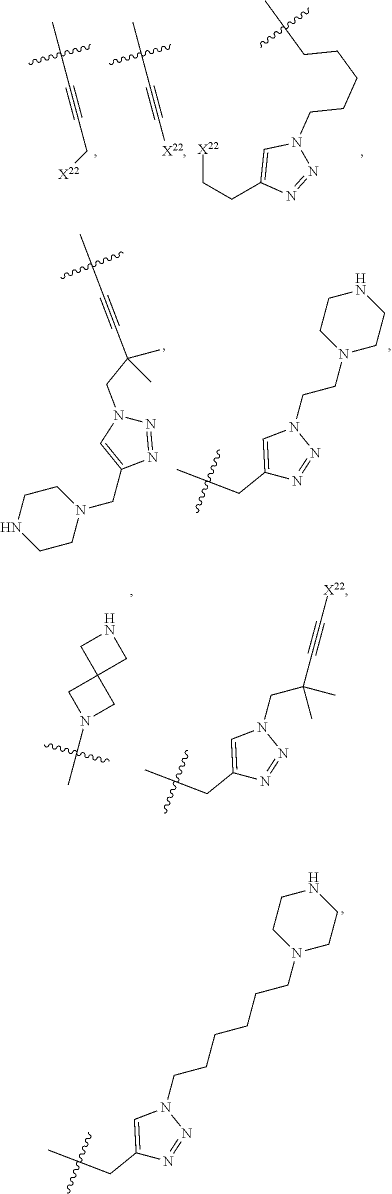

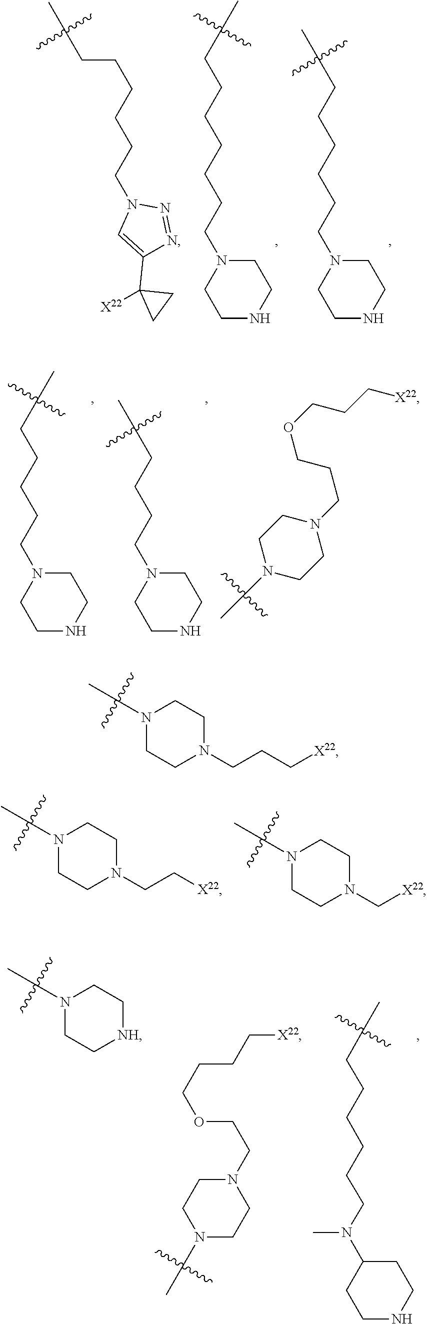

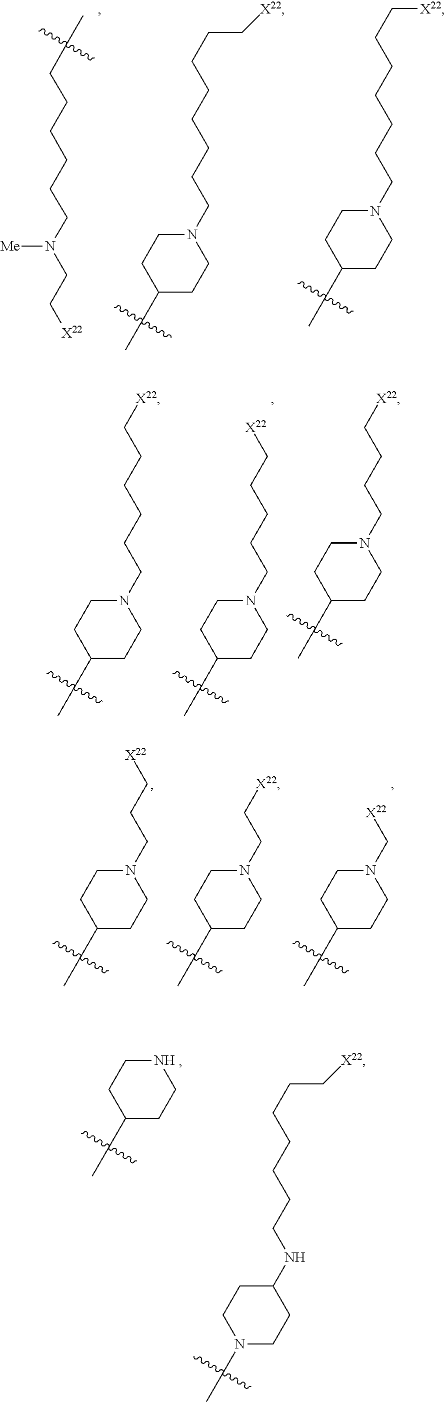

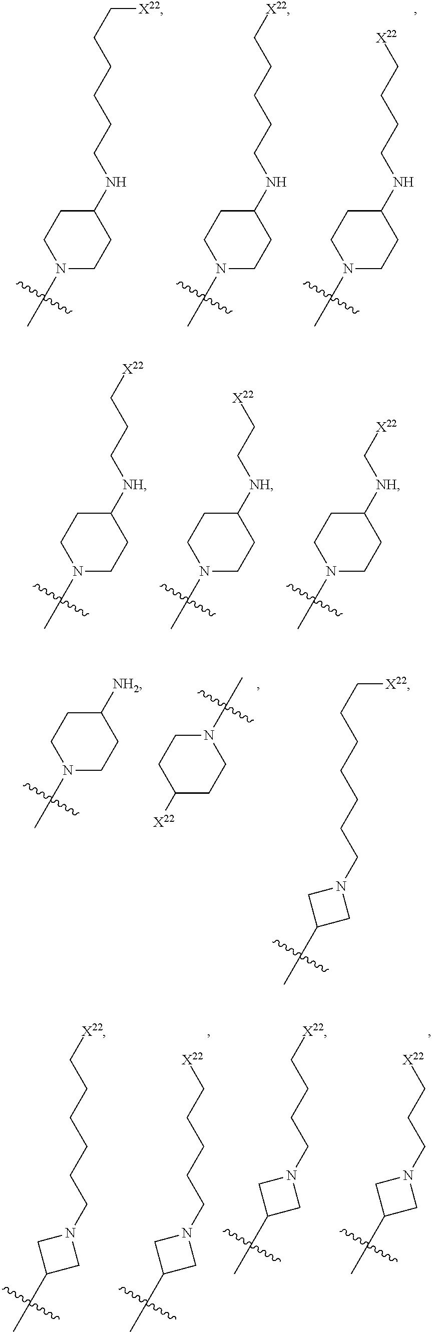

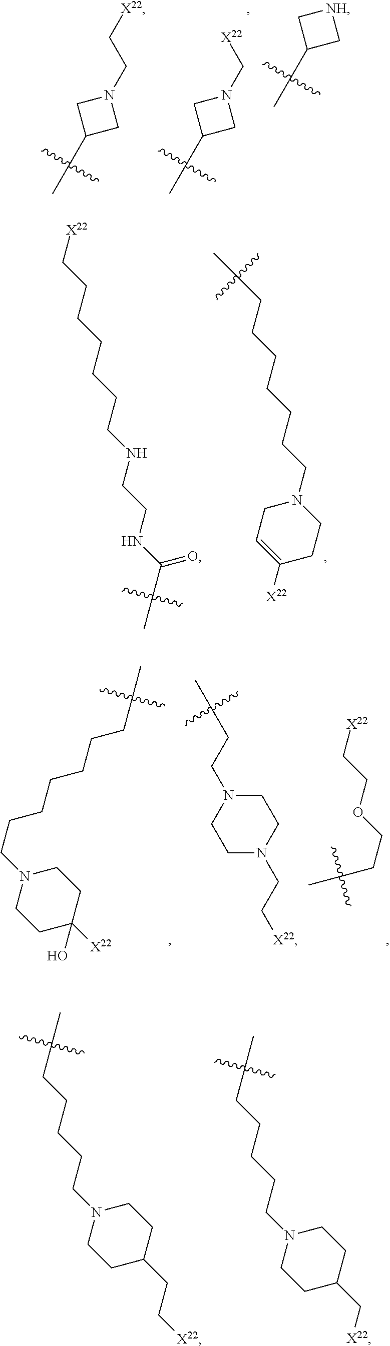

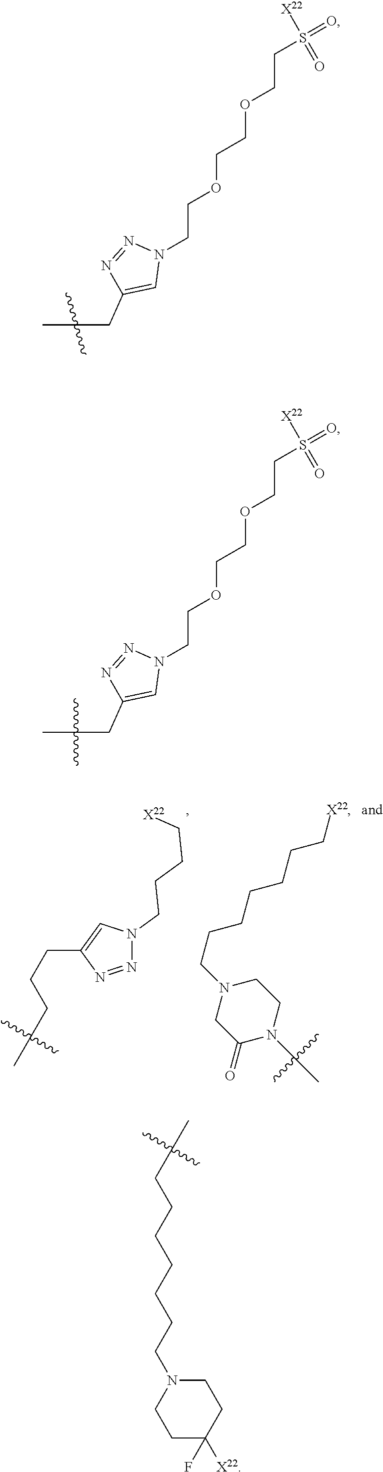

In additional embodiments, Tail is selected from the group consisting of:

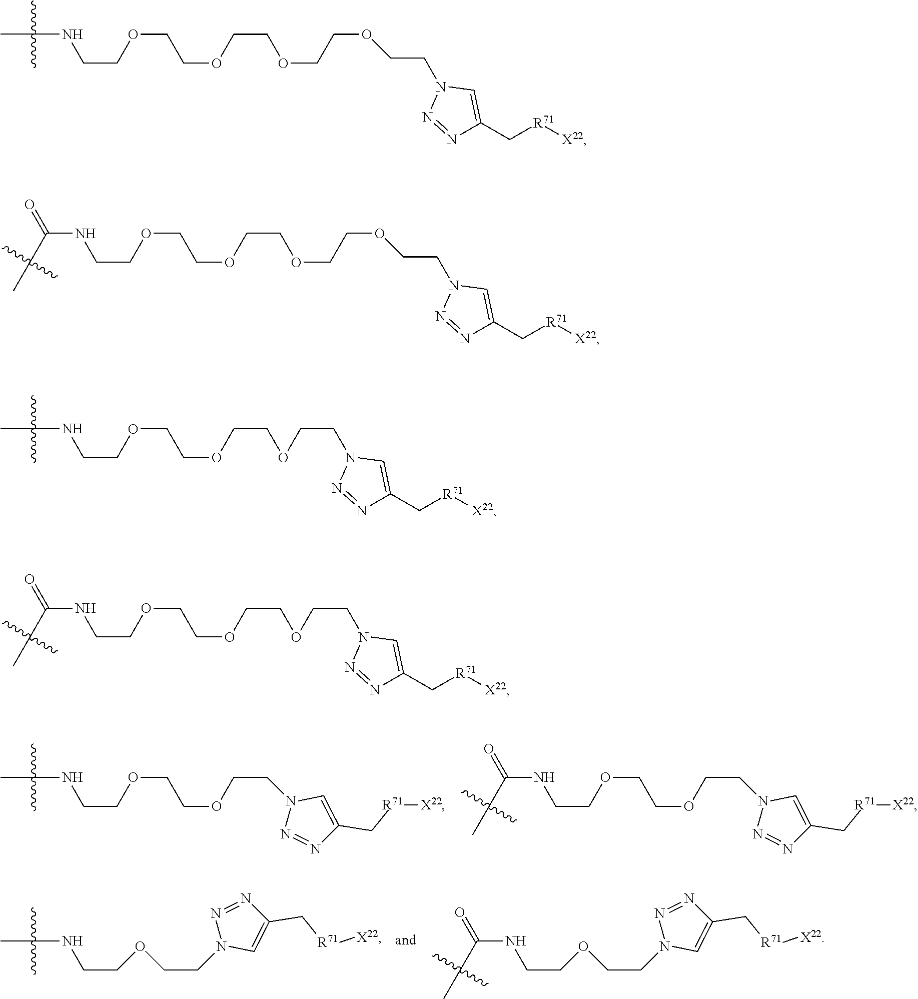

In additional embodiments, Tail is selected from the group consisting of:

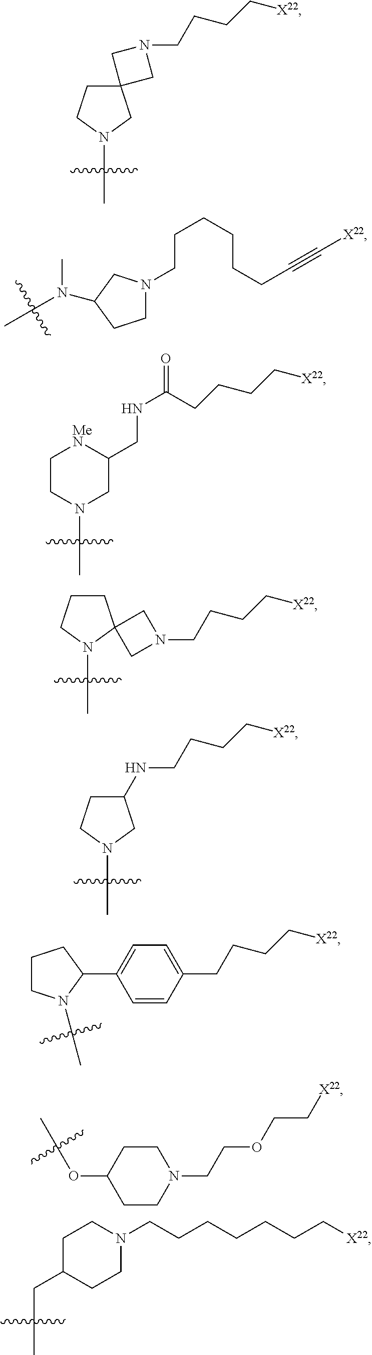

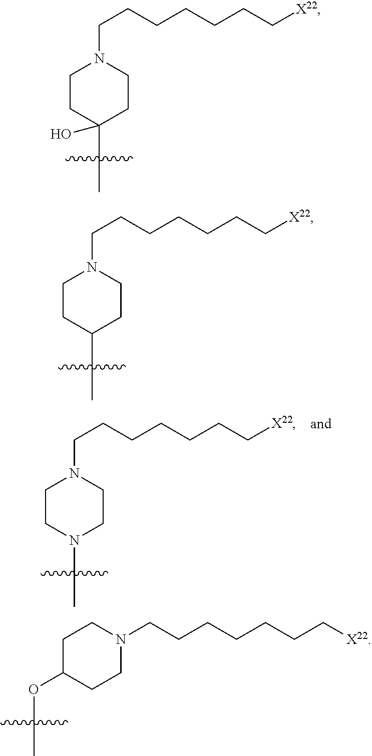

In additional embodiments, Tail is selected from the group consisting of:

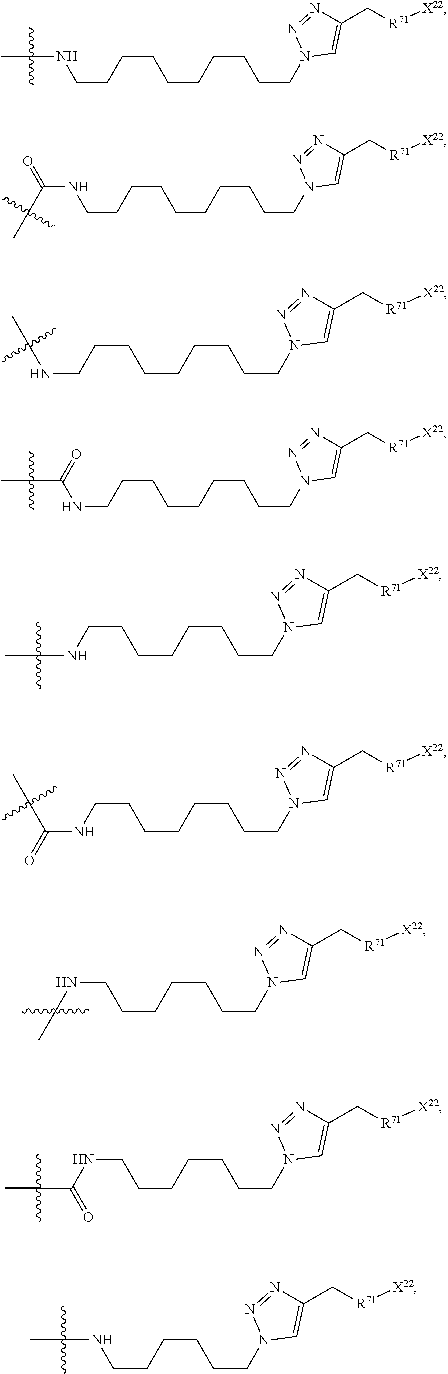

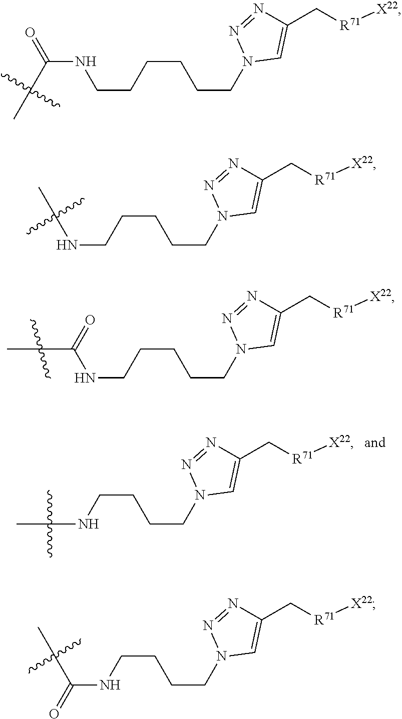

wherein R⁷¹ is —O—, —NH, Nalkyl, alkyl, or -NMe.

In additional embodiments, Tail is selected from the group consisting of:

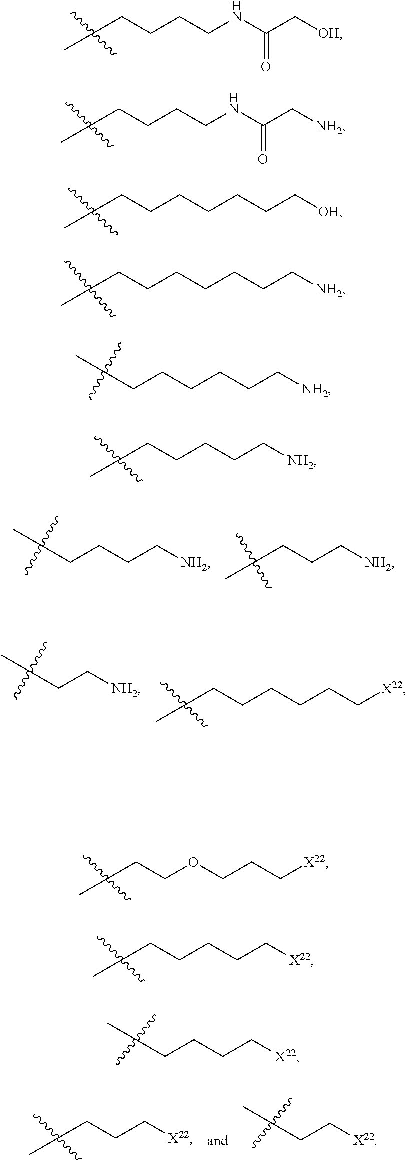

In additional embodiments, Tail is selected from the group consisting of:

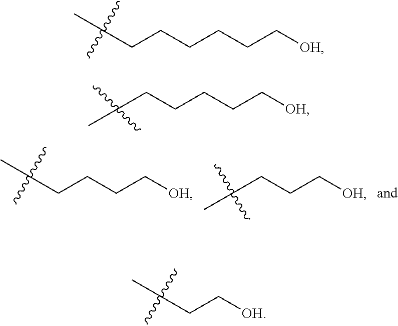

In certain embodiments, Tail is selected from the group consisting of:

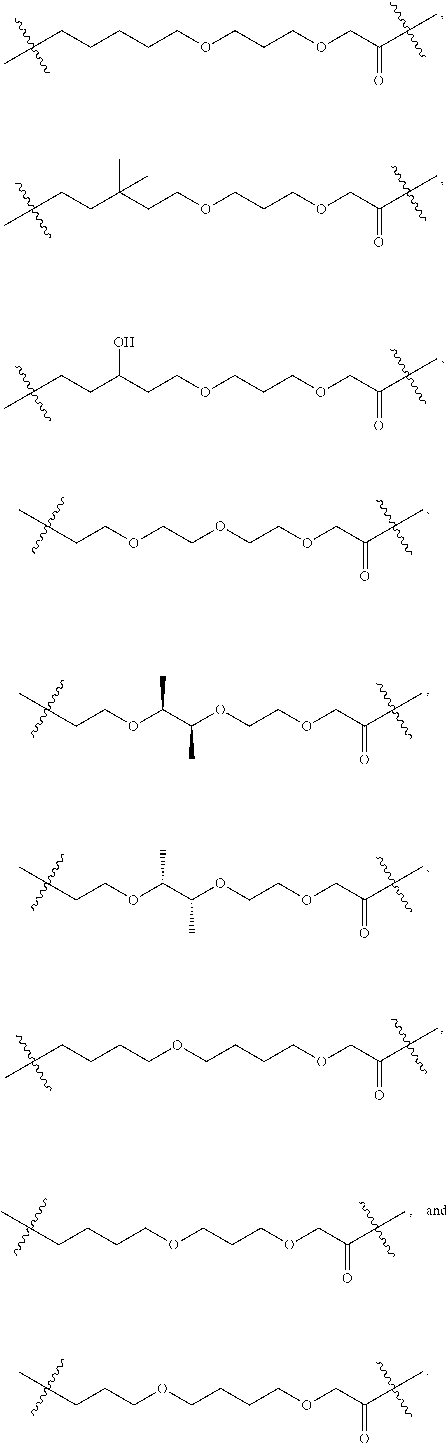

In certain embodiments, Tail is selected from the group consisting of:

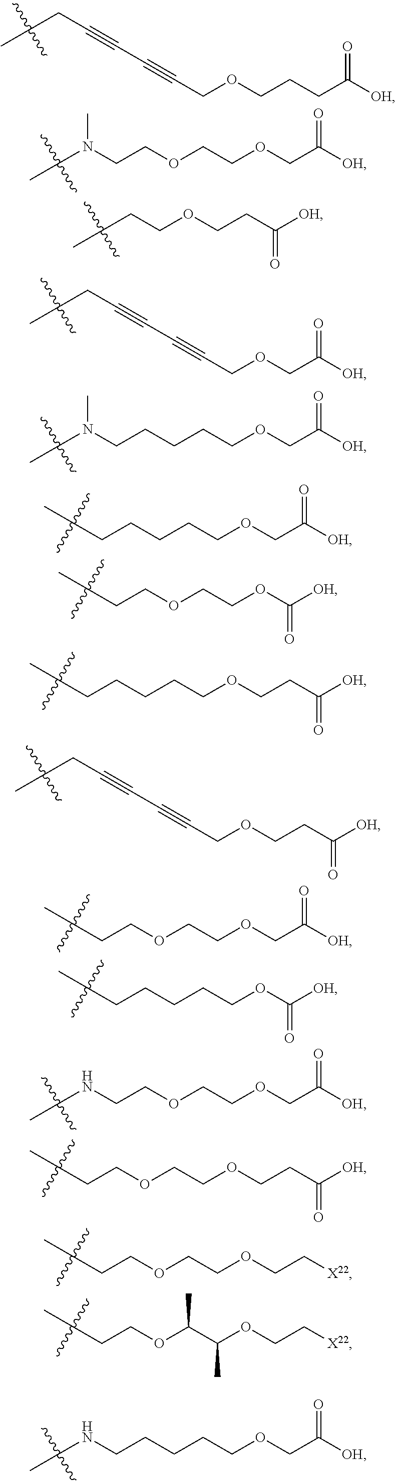

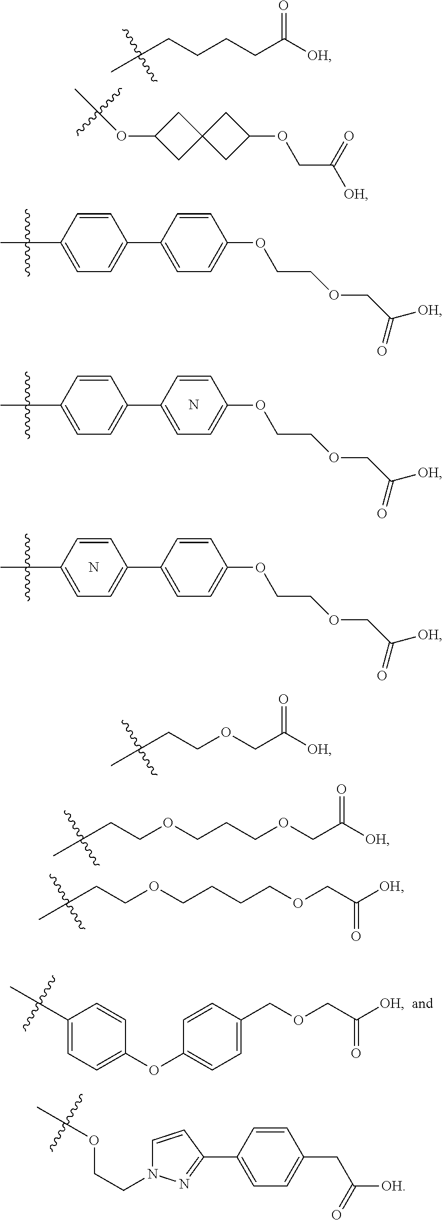

In the above structures

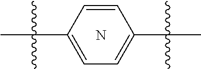

represents

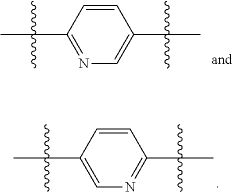 [SMILES: *c1ccc(*)nc1 ; *c1ccc(*)nc1]

In certain embodiments, Tail can be a 4-24 carbon atom linear chains, wherein one or more the carbon atoms in the linear chain can be replaced or substituted with oxygen, nitrogen, amide, fluorinated carbon, etc., such as the following.

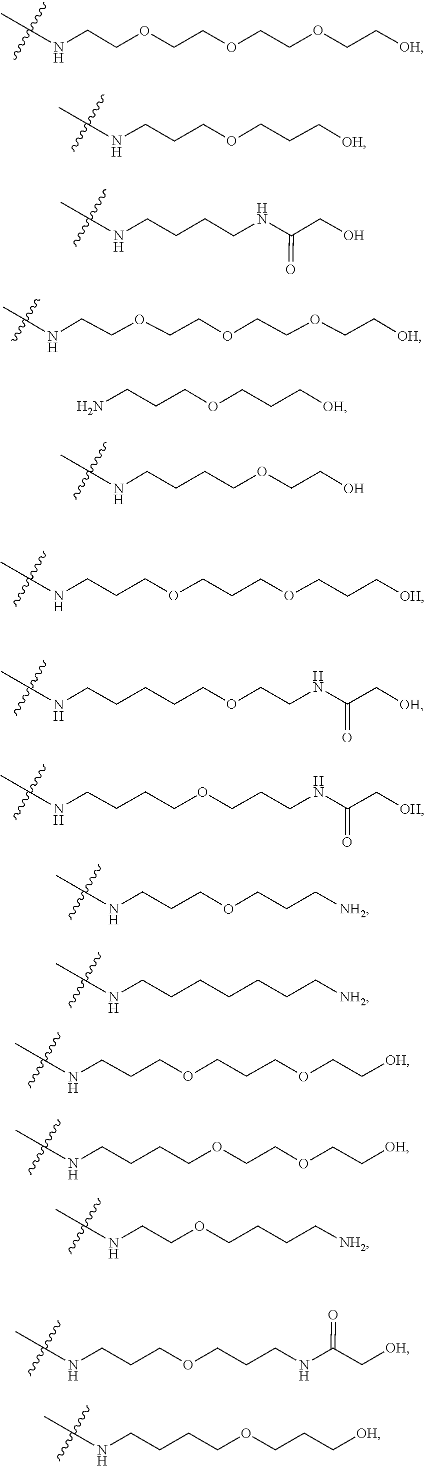

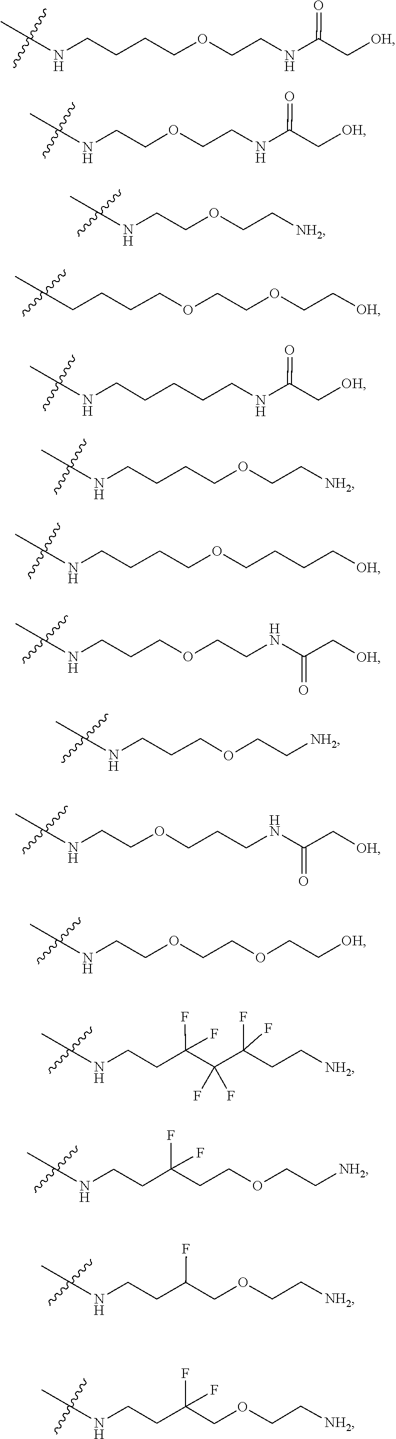

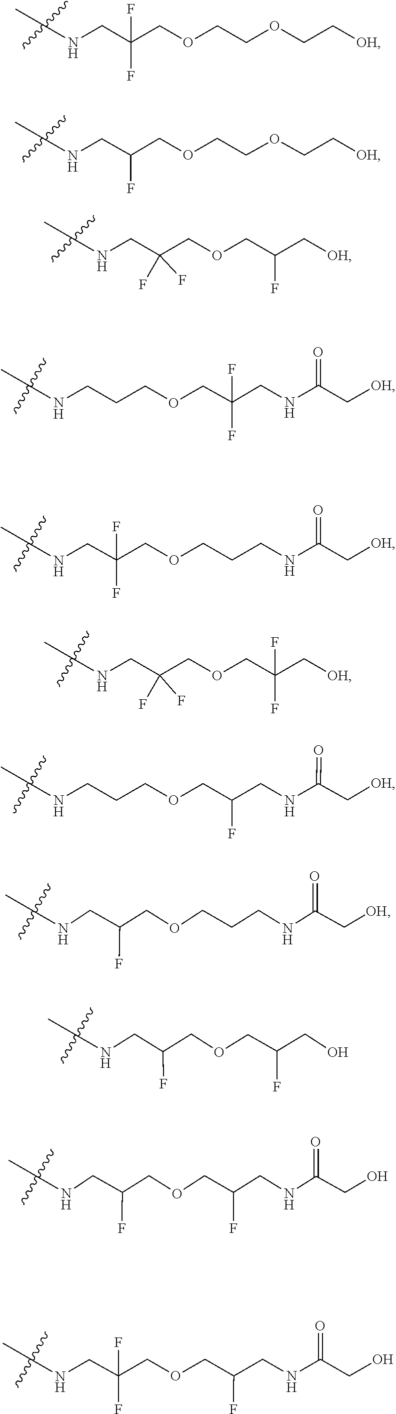

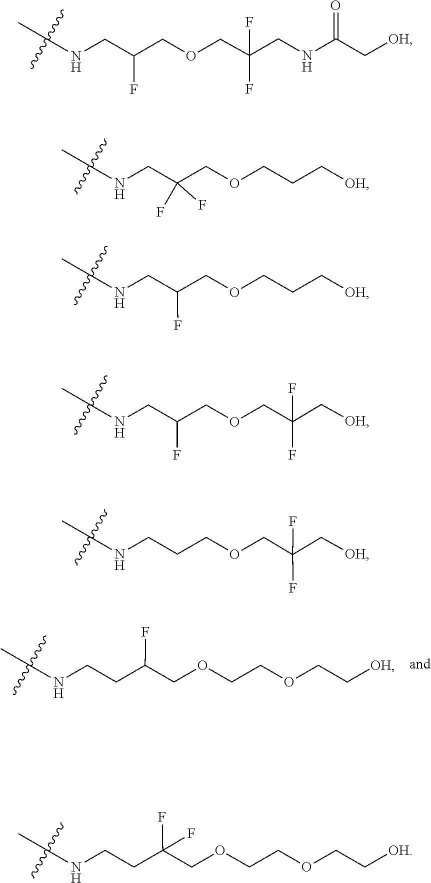

In certain embodiments, Tai may include contiguous, partially contiguous or non-contiguous ethylene glycol unit groups ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units, for example, 1, 2, 3, 4, 6, 6, 7, 8, 9, 10, 11 or 12 ethylene glycol units.

In certain embodiments, Tail may have 1, 2, 3, 4, 5, 6, 7, 8, 9, 10, 11, 12, 13, 14, or 15 fluorine substituents. In another embodiment Tail is perfluorinated. In yet another embodiment -Tail is a partially or fully fluorinated poly ether. Nonlimiting examples of fluorinated Tail moieties include:

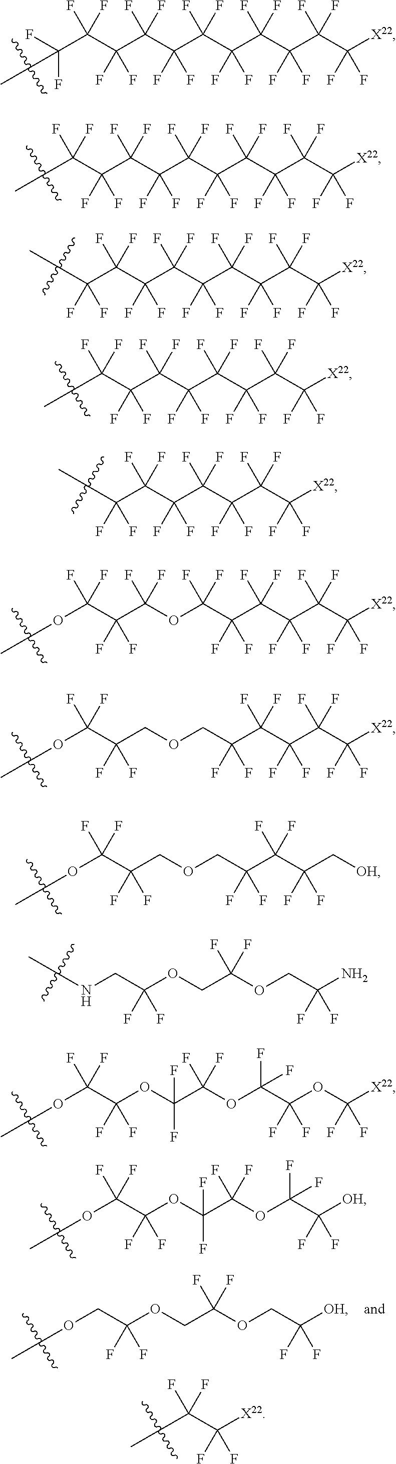

In certain embodiments, the length can be adjusted as desired or as found necessary for the desired application.

In certain embodiments, Linker is selected from the group consisting of:

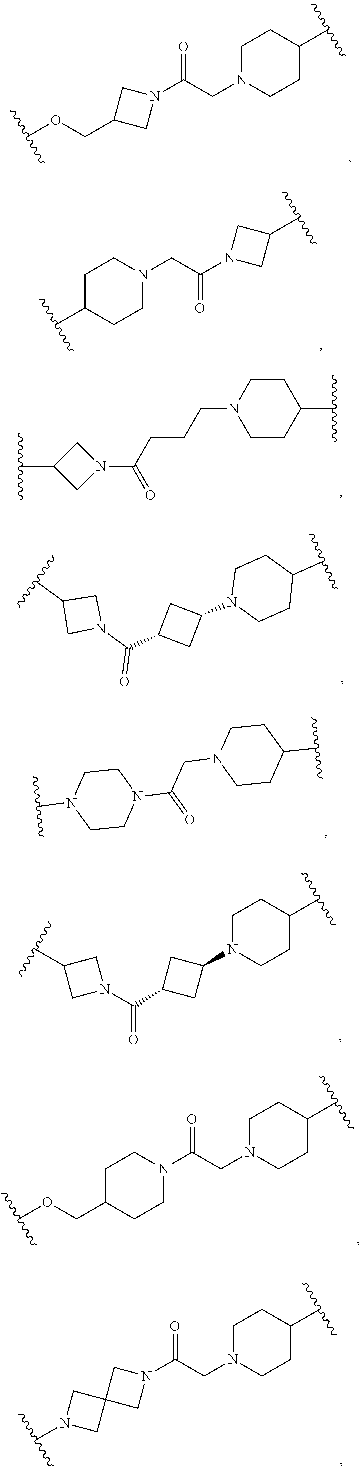

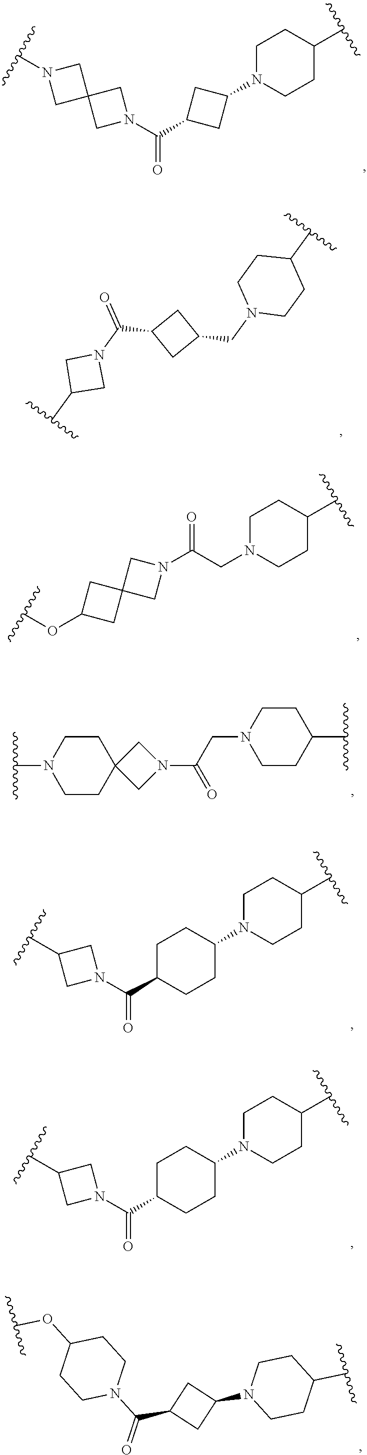

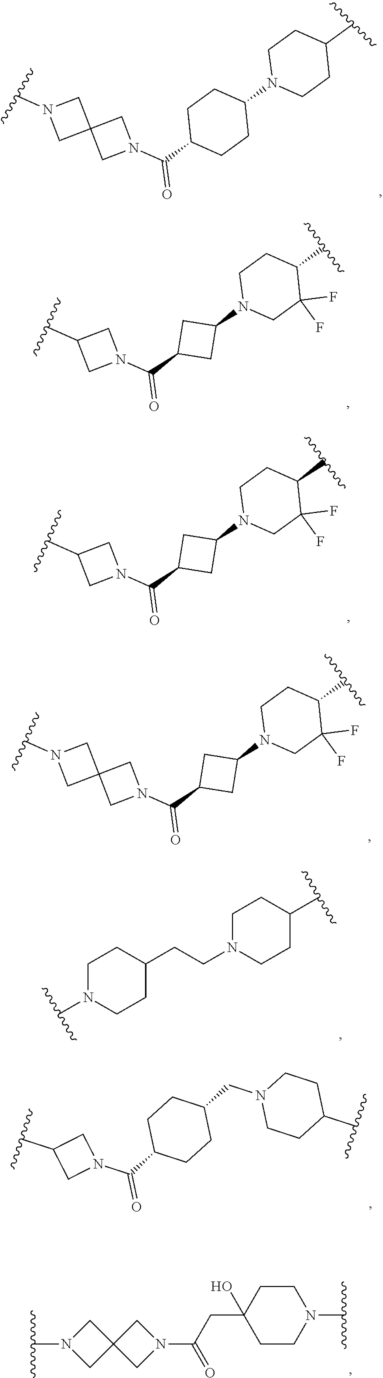

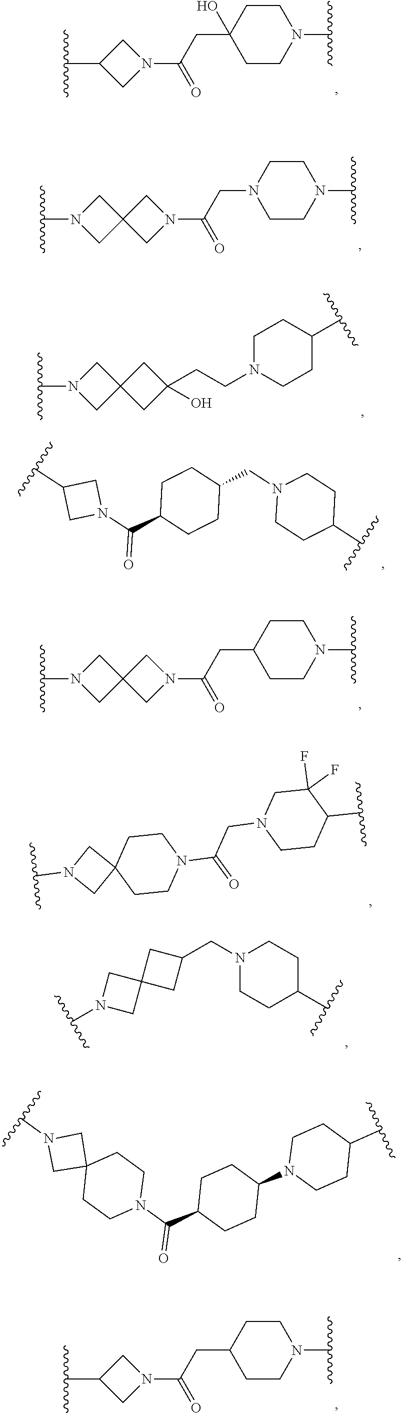

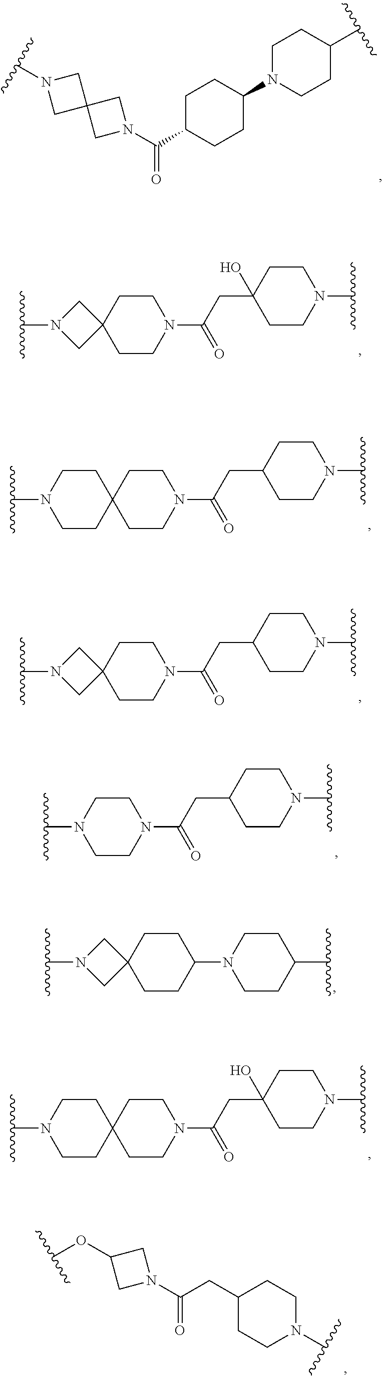

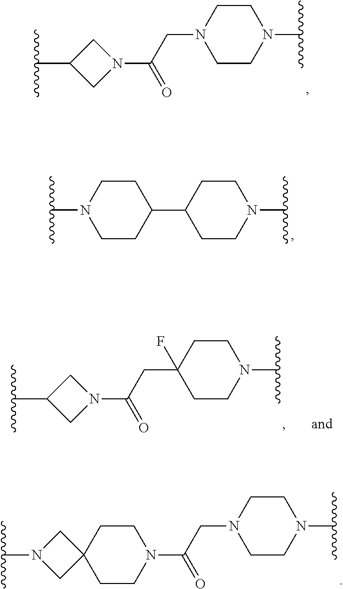 [SMILES: *C1CN(C(=O)CN2CCN(*)CC2)C1 ; *N1CCC(C2CCN(*)CC2)CC1 ; *C1CN(C(=O)CC2(F)CCN(*)CC2)C1 ; *N1CCN(CC(=O)N2CCC3(CC2)CN(*)C3)CC1]

In certain embodiments, Linker is selected from the group consisting of:

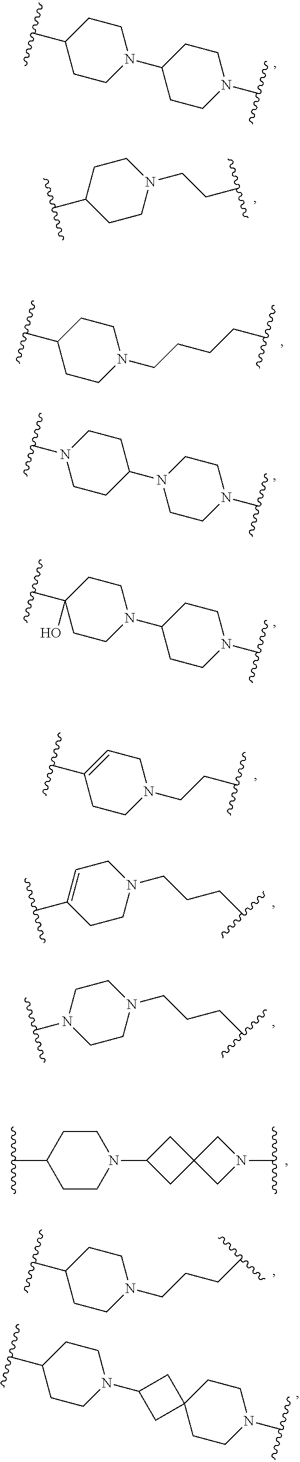

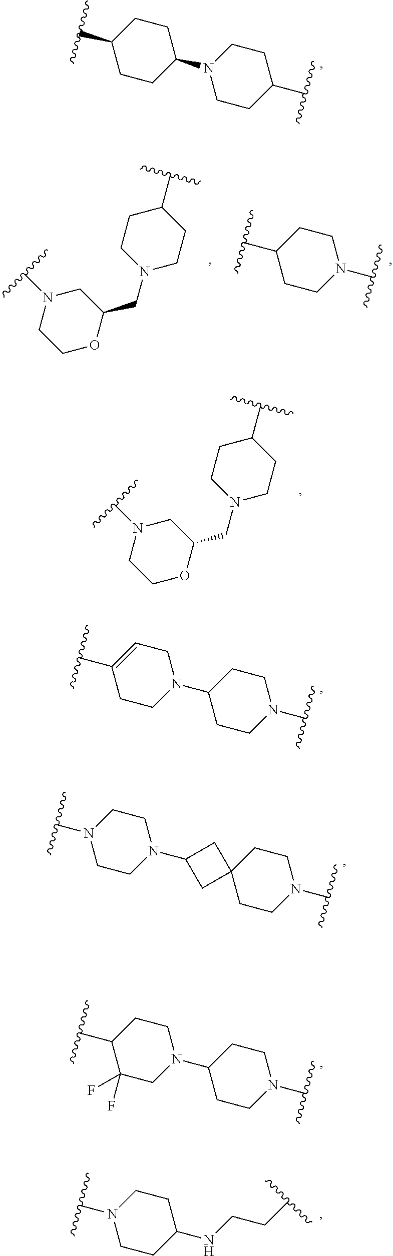

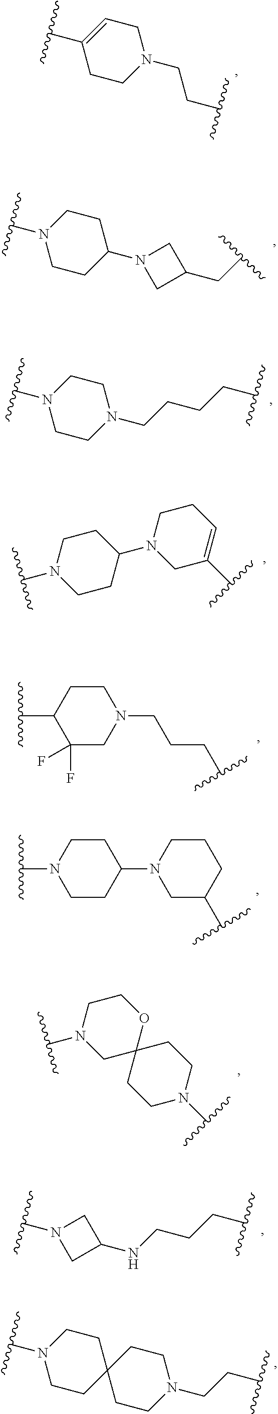

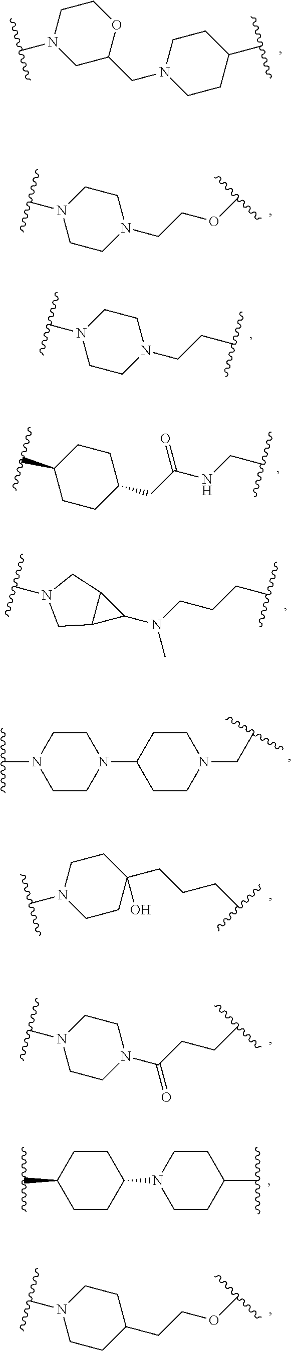

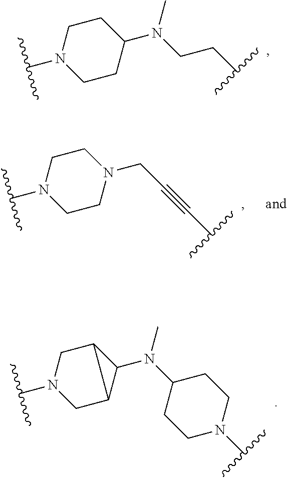 [SMILES: *CCN(C)C1CCN(*)CC1 ; *C#CCN1CCN(*)CC1 ; *N1CCC(N(C)C2C3CN(*)CC32)CC1]

In certain embodiments, Linker is selected from the group consisting of:

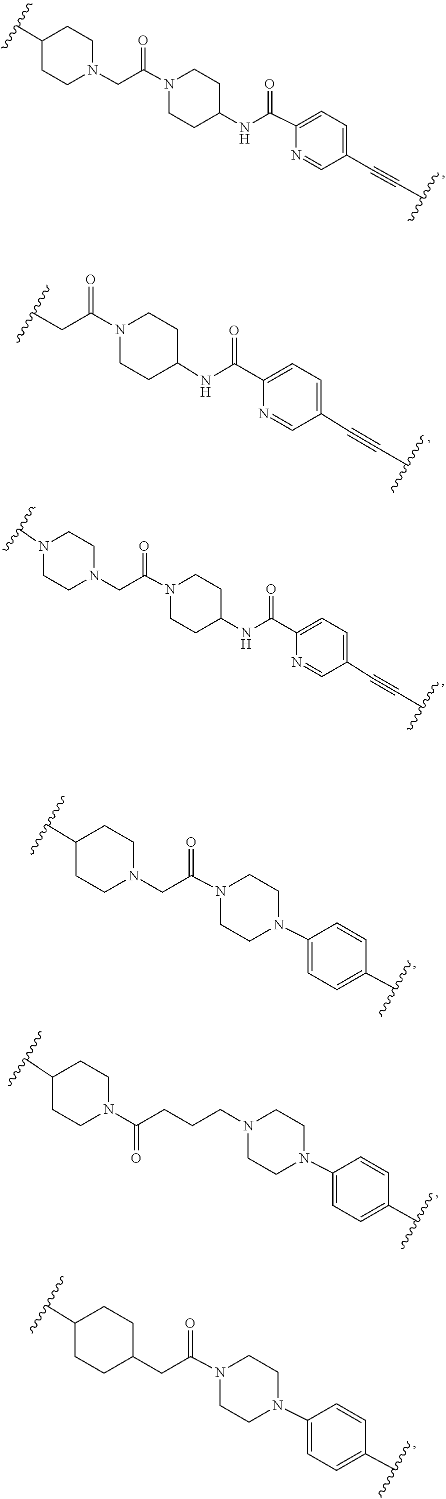 [SMILES: *C#Cc1ccc(C(=O)NC2CCN(C(=O)CN3CCC(*)CC3)CC2)nc1 ; *C#Cc1ccc(C(=O)NC2CCN(C(=O)C*)CC2)nc1 ; *C#Cc1ccc(C(=O)NC2CCN(C(=O)CN3CCN(*)CC3)CC2)nc1 ; *c1ccc(N2CCN(C(=O)CN3CCC(*)CC3)CC2)cc1 ; *c1ccc(N2CCN(CCCC(=O)N3CCC(*)CC3)CC2)cc1 ; *c1ccc(N2CCN(C(=O)CC3CCC(*)CC3)CC2)cc1]

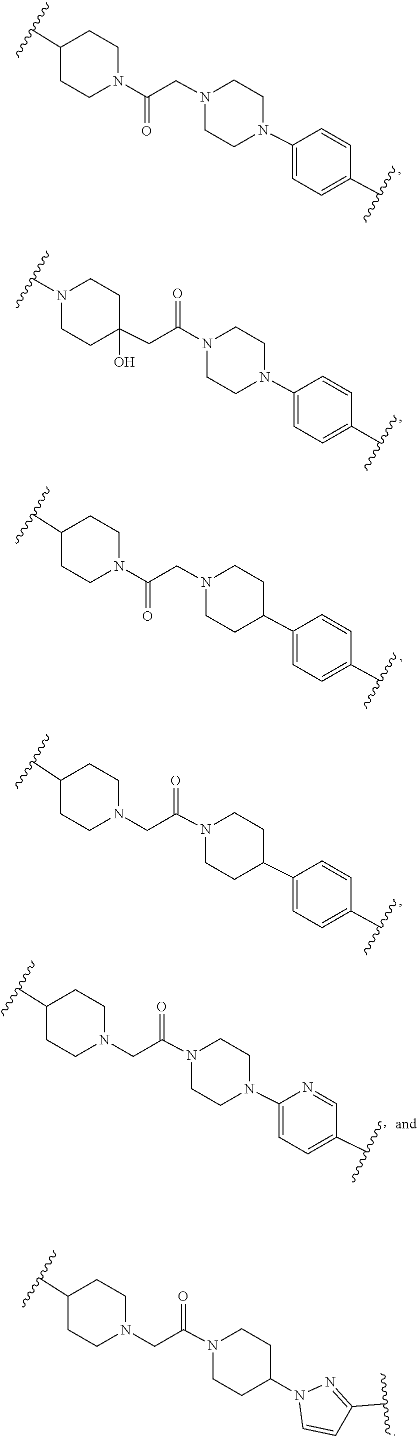 [SMILES: *c1ccc(N2CCN(CC(=O)N3CCC(*)CC3)CC2)cc1 ; *c1ccc(N2CCN(C(=O)CC3(O)CCN(*)CC3)CC2)cc1 ; *c1ccc(C2CCN(CC(=O)N3CCC(*)CC3)CC2)cc1 ; *c1ccc(C2CCN(C(=O)CN3CCC(*)CC3)CC2)cc1 ; *c1ccc(N2CCN(C(=O)CN3CCC(*)CC3)CC2)nc1 ; *c1ccn(C2CCN(C(=O)CN3CCC(*)CC3)CC2)n1]

In certain embodiments, Linker is selected from the group consisting of:

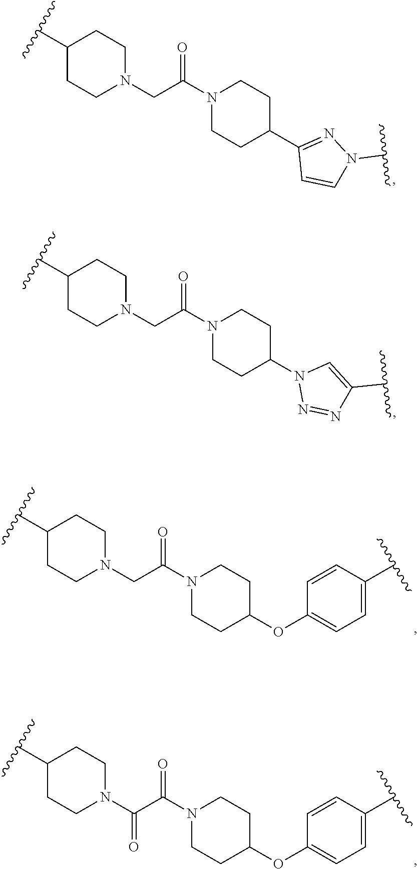 [SMILES: *C1CCN(CC(=O)N2CCC(c3ccn(*)n3)CC2)CC1 ; *c1cn(C2CCN(C(=O)CN3CCC(*)CC3)CC2)nn1 ; *c1ccc(OC2CCN(C(=O)CN3CCC(*)CC3)CC2)cc1 ; *c1ccc(OC2CCN(C(=O)C(=O)N3CCC(*)CC3)CC2)cc1]

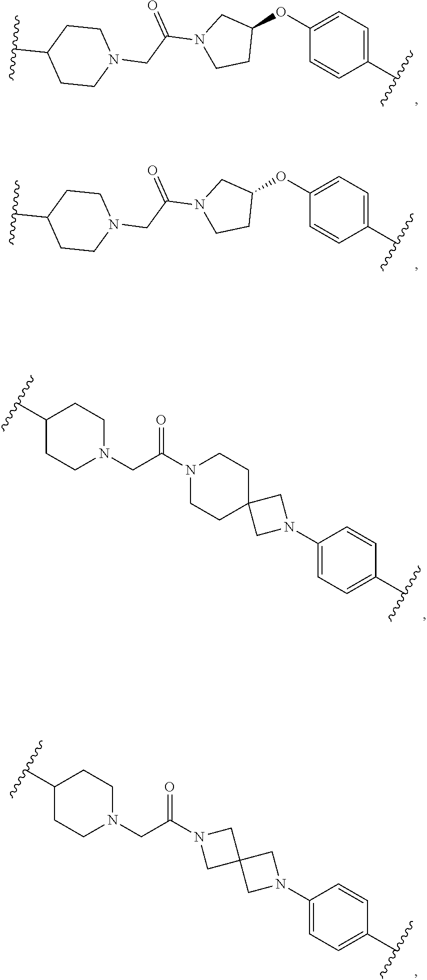 [SMILES: *c1ccc(O[C@H]2CCN(C(=O)CN3CCC(*)CC3)C2)cc1 ; *c1ccc(O[C@@H]2CCN(C(=O)CN3CCC(*)CC3)C2)cc1 ; *c1ccc(N2CC3(CCN(C(=O)CN4CCC(*)CC4)CC3)C2)cc1 ; *c1ccc(N2CC3(CN(C(=O)CN4CCC(*)CC4)C3)C2)cc1]

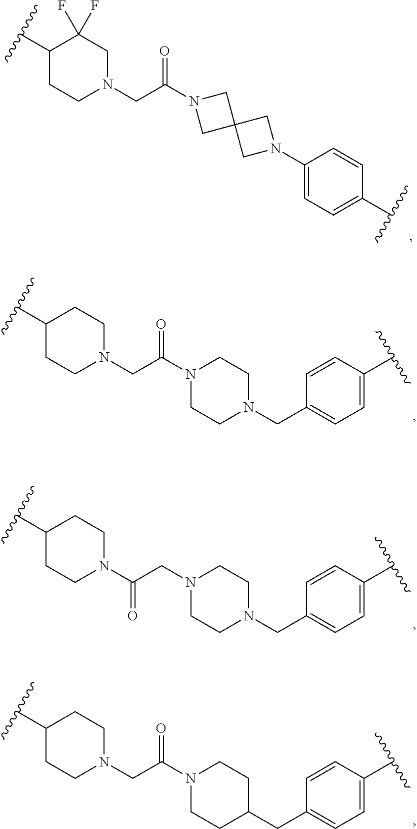 [SMILES: *c1ccc(N2CC3(CN(C(=O)CN4CCC(*)C(F)(F)C4)C3)C2)cc1 ; *c1ccc(CN2CCN(C(=O)CN3CCC(*)CC3)CC2)cc1 ; *c1ccc(CN2CCN(CC(=O)N3CCC(*)CC3)CC2)cc1 ; *c1ccc(CC2CCN(C(=O)CN3CCC(*)CC3)CC2)cc1]

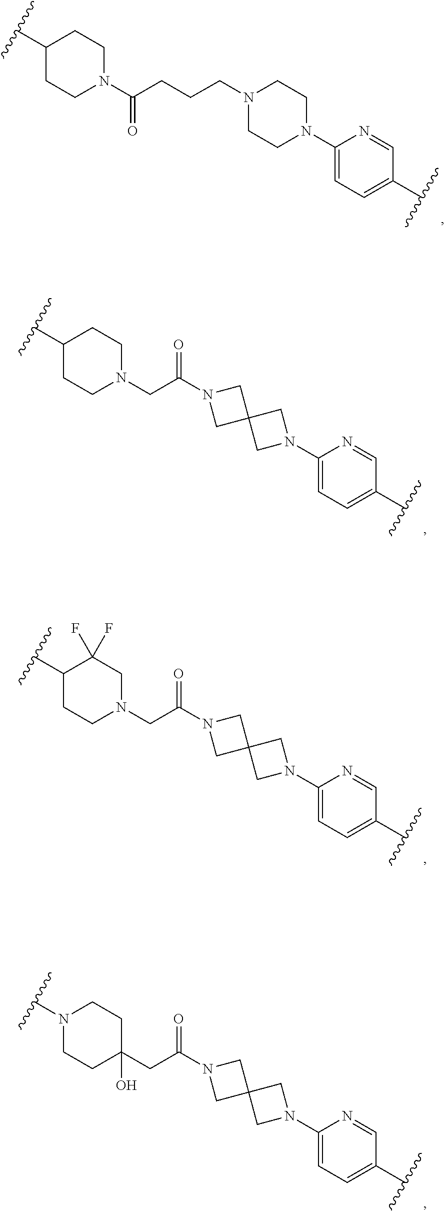 [SMILES: *c1ccc(N2CCN(CCCC(=O)N3CCC(*)CC3)CC2)nc1 ; *c1ccc(N2CC3(CN(C(=O)CN4CCC(*)CC4)C3)C2)nc1 ; *c1ccc(N2CC3(CN(C(=O)CN4CCC(*)C(F)(F)C4)C3)C2)nc1 ; *c1ccc(N2CC3(CN(C(=O)CC4(O)CCN(*)CC4)C3)C2)nc1]

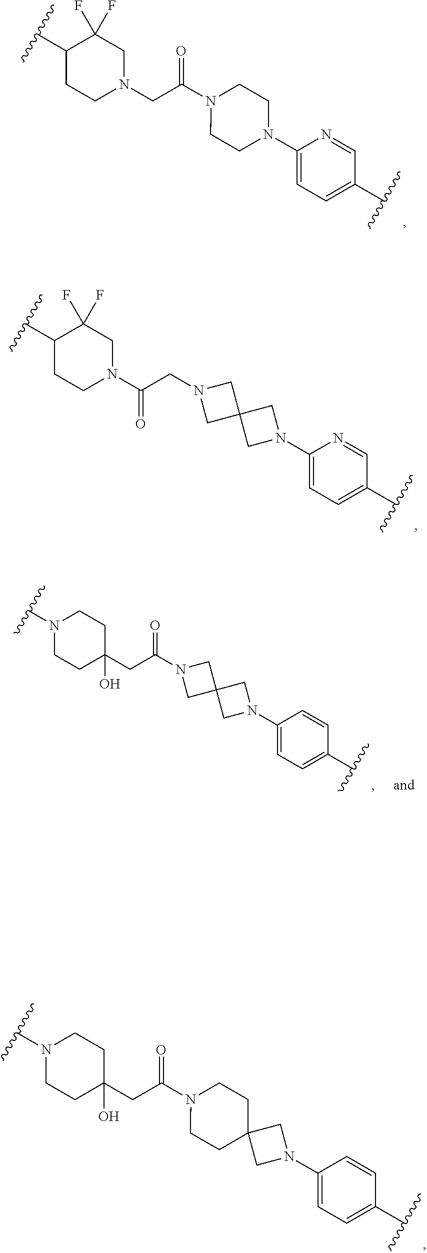 [SMILES: *c1ccc(N2CCN(C(=O)CN3CCC(*)C(F)(F)C3)CC2)nc1 ; *c1ccc(N2CC3(CN(CC(=O)N4CCC(*)C(F)(F)C4)C3)C2)nc1 ; *c1ccc(N2CC3(CN(C(=O)CC4(O)CCN(*)CC4)C3)C2)cc1 ; *c1ccc(N2CC3(CCN(C(=O)CC4(O)CCN(*)CC4)CC3)C2)cc1]

In certain embodiments, Linker is selected from the group consisting of:

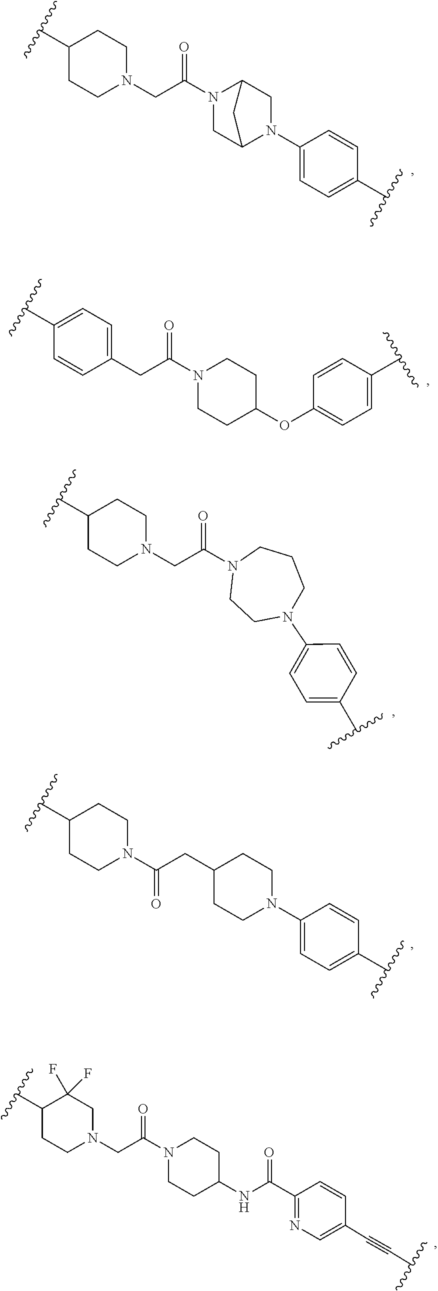 [SMILES: *c1ccc(N2CC3CC2CN3C(=O)CN2CCC(*)CC2)cc1 ; *c1ccc(CC(=O)N2CCC(Oc3ccc(*)cc3)CC2)cc1 ; *c1ccc(N2CCCN(C(=O)CN3CCC(*)CC3)CC2)cc1 ; *c1ccc(N2CCC(CC(=O)N3CCC(*)CC3)CC2)cc1 ; *C#Cc1ccc(C(=O)NC2CCN(C(=O)CN3CCC(*)C(F)(F)C3)CC2)nc1]

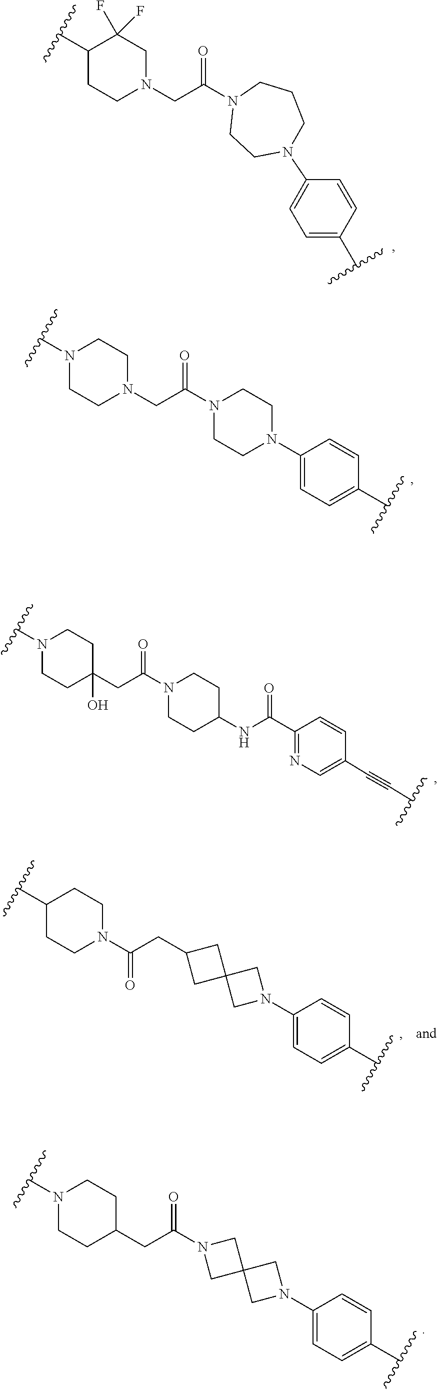 [SMILES: *c1ccc(N2CCCN(C(=O)CN3CCC(*)C(F)(F)C3)CC2)cc1 ; *c1ccc(N2CCN(C(=O)CN3CCN(*)CC3)CC2)cc1 ; *C#Cc1ccc(C(=O)NC2CCN(C(=O)CC3(O)CCN(*)CC3)CC2)nc1 ; *c1ccc(N2CC3(CC(CC(=O)N4CCC(*)CC4)C3)C2)cc1 ; *c1ccc(N2CC3(CN(C(=O)CC4CCN(*)CC4)C3)C2)cc1]

In certain embodiments, Linker is selected from the group consisting of:

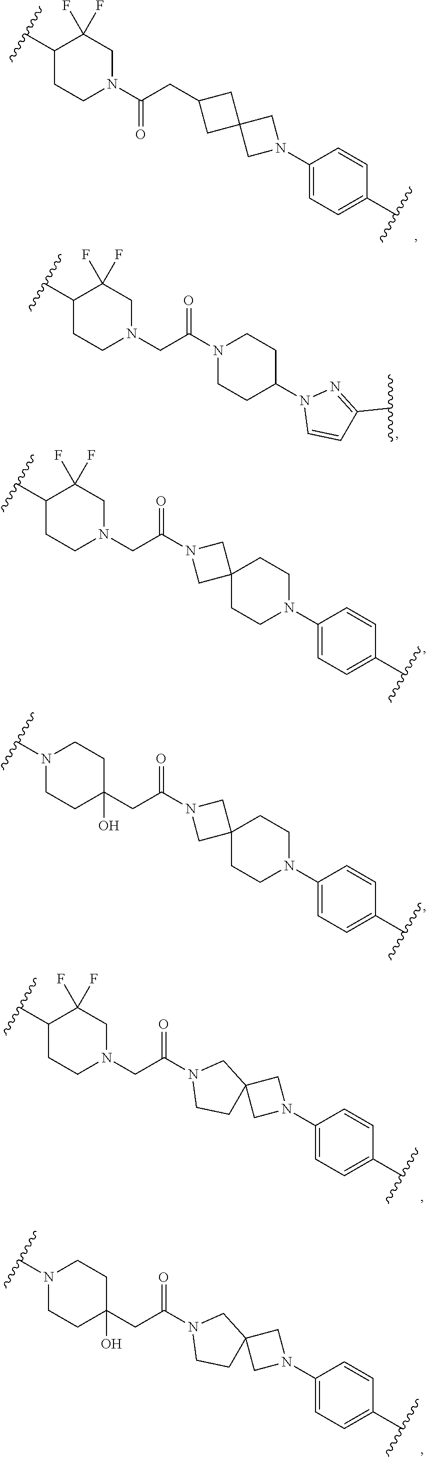 [SMILES: *c1ccc(N2CC3(CC(CC(=O)N4CCC(*)C(F)(F)C4)C3)C2)cc1 ; *c1ccn(C2CCN(C(=O)CN3CCC(*)C(F)(F)C3)CC2)n1 ; *c1ccc(N2CCC3(CC2)CN(C(=O)CN2CCC(*)C(F)(F)C2)C3)cc1 ; *c1ccc(N2CCC3(CC2)CN(C(=O)CC2(O)CCN(*)CC2)C3)cc1 ; *c1ccc(N2CC3(CCN(C(=O)CN4CCC(*)C(F)(F)C4)C3)C2)cc1 ; *c1ccc(N2CC3(CCN(C(=O)CC4(O)CCN(*)CC4)C3)C2)cc1]

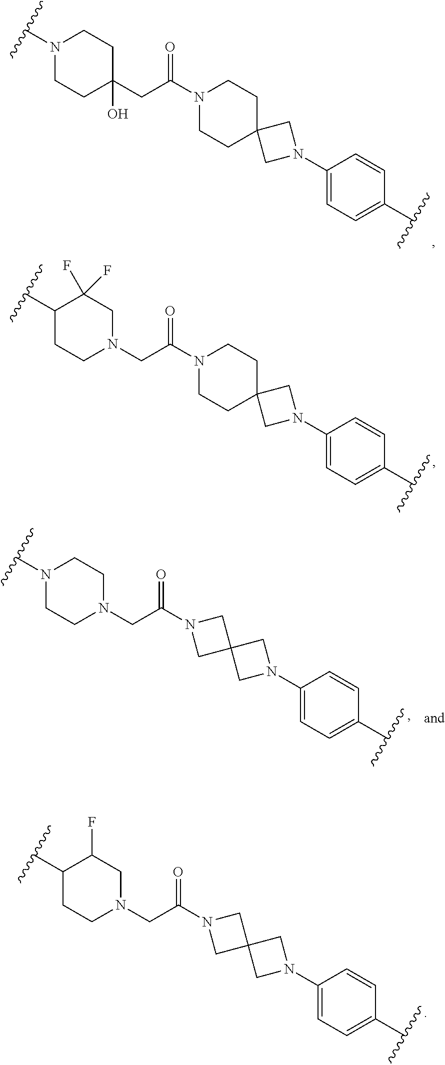 [SMILES: *c1ccc(N2CC3(CCN(C(=O)CC4(O)CCN(*)CC4)CC3)C2)cc1 ; *c1ccc(N2CC3(CCN(C(=O)CN4CCC(*)C(F)(F)C4)CC3)C2)cc1 ; *c1ccc(N2CC3(CN(C(=O)CN4CCN(*)CC4)C3)C2)cc1 ; *c1ccc(N2CC3(CN(C(=O)CN4CCC(*)C(F)C4)C3)C2)cc1]

In certain embodiments, Linker is selected from the group consisting of:

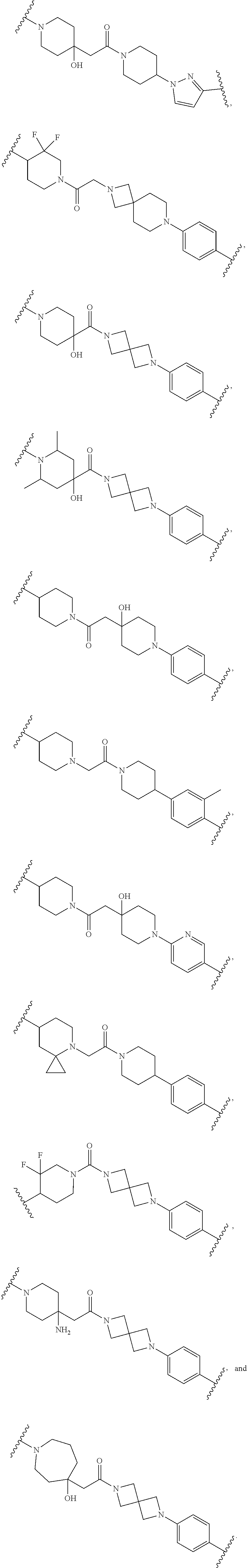

In certain embodiments, Linker is selected from the group consisting of:

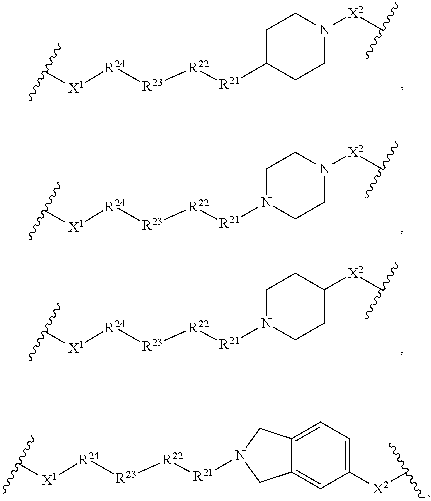

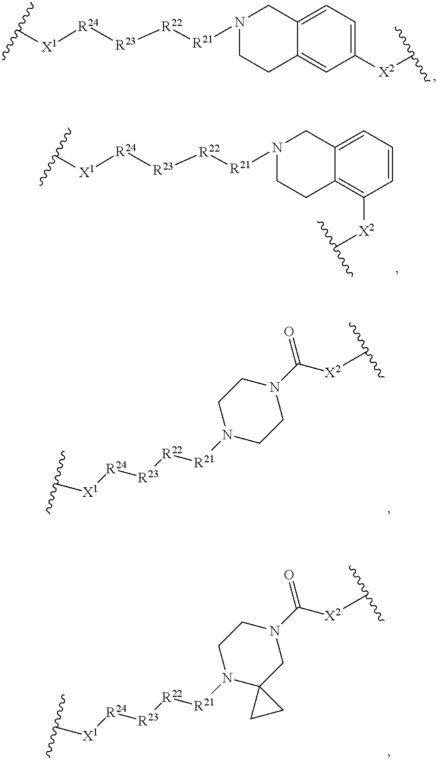

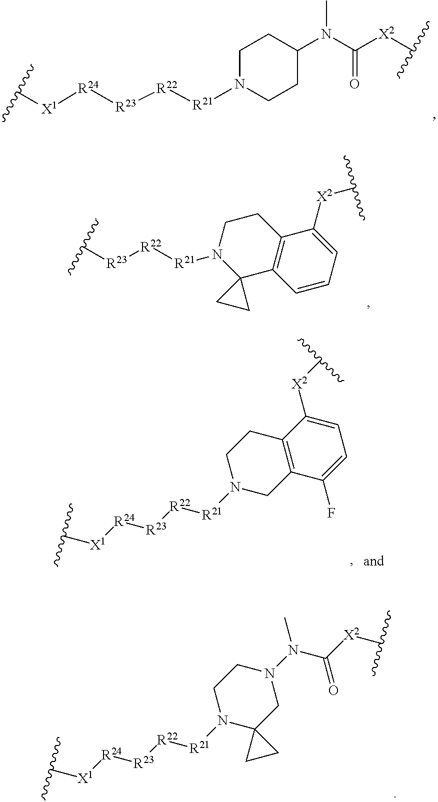

In certain embodiments, Linker is selected from the group consisting of:

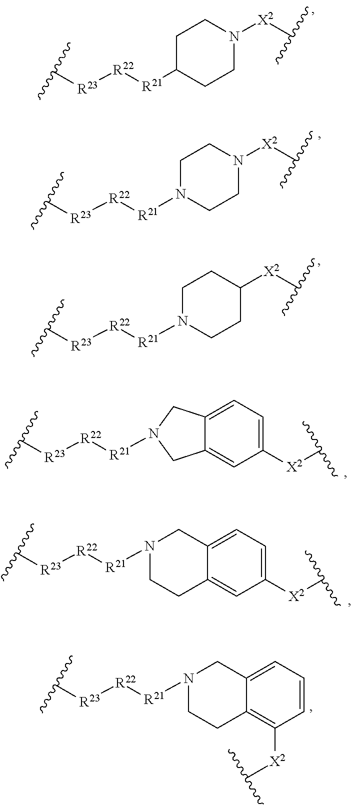

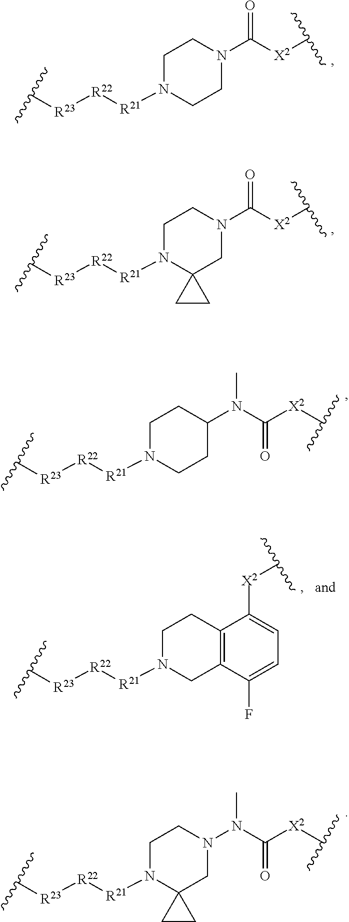

Non-limiting examples of moieties of R²⁰, R²¹, R²², R²³, and R²⁴ include:

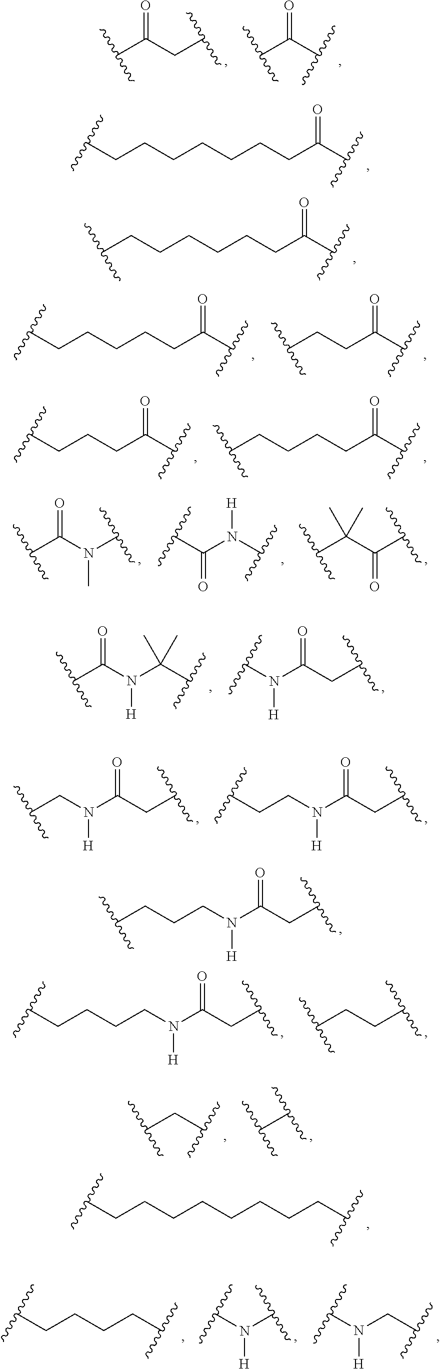

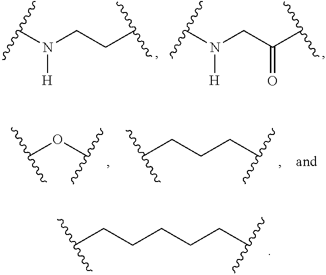

Additional non-limiting examples of moieties of R²⁰, R²¹, R²², R²³ and R²⁴ include:

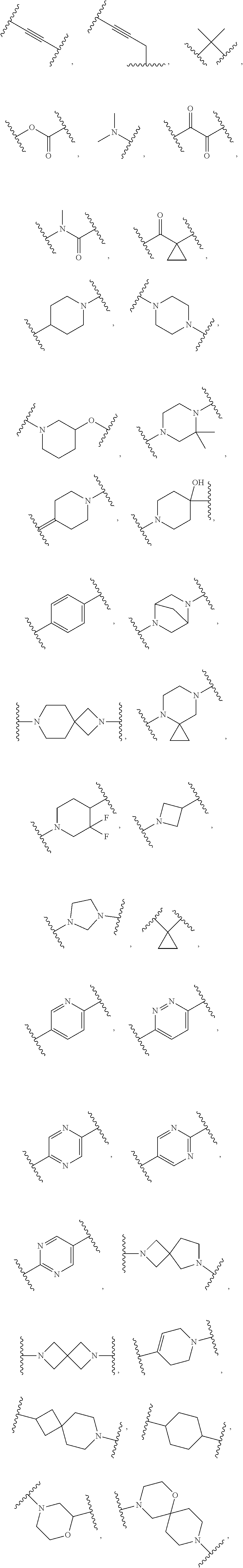

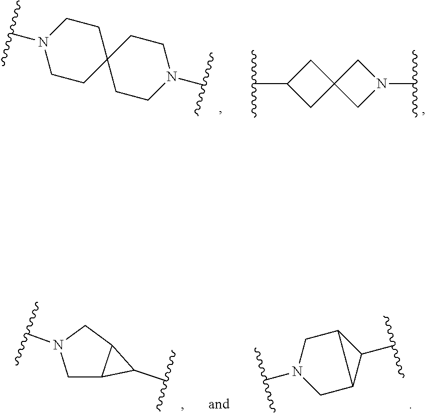 [SMILES: *N1CCC2(CC1)CCN(*)CC2 ; *C1CC2(C1)CN(*)C2 ; *C1C2CN(*)CC12 ; *C1C2CN(*)CC12]

Additional non-limiting examples of moieties of R²⁰, R²¹, R²², R²³ and R²⁴ include:

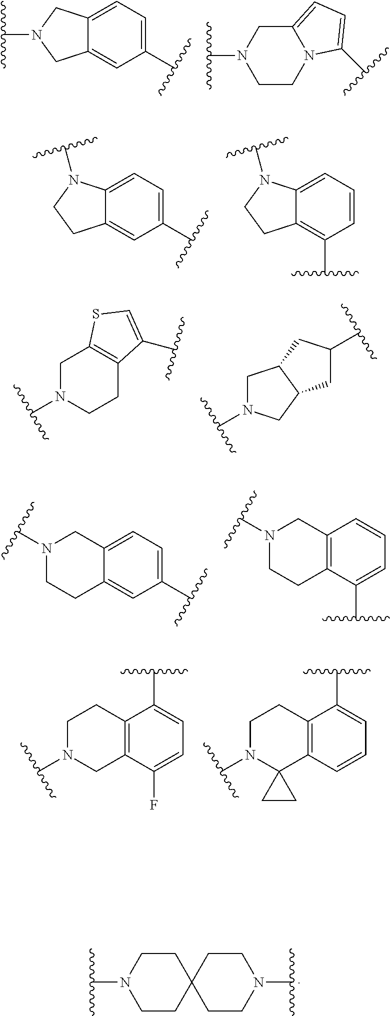

In additional embodiments, the Linker moiety is an optionally substituted (poly)ethylene glycol having at least 1, at least 2, at least 3, at least 4, at least 5, at least 6, at least 7, at least 8, at least 9, at least 10, ethylene glycol units, or optionally substituted alkyl groups interspersed with optionally substituted, O, N, S, P or Si atoms.

In certain embodiments, the Linker is flanked, substituted, or interspersed with an aryl, phenyl, benzyl, alkyl, alkylene, or heterocycle group.

In certain embodiments, the Linker may be asymmetric or symmetrical.

In any of the embodiments of a compound of the present invention, the Linker group may be any suitable moiety as described herein.

In certain embodiments, the Linker is selected from the group consisting of:

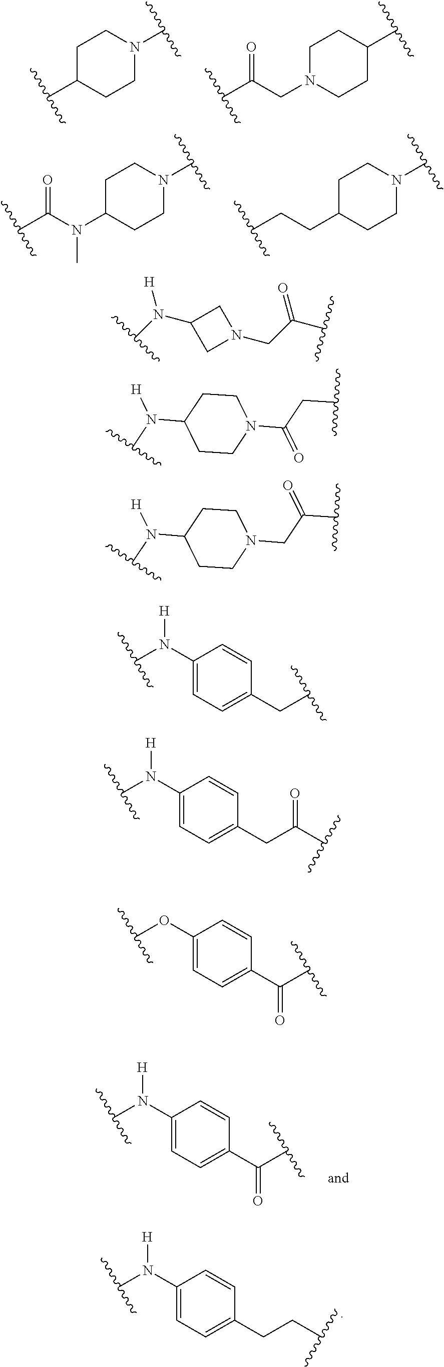

In certain embodiments, the Linker is selected from the group consisting of:

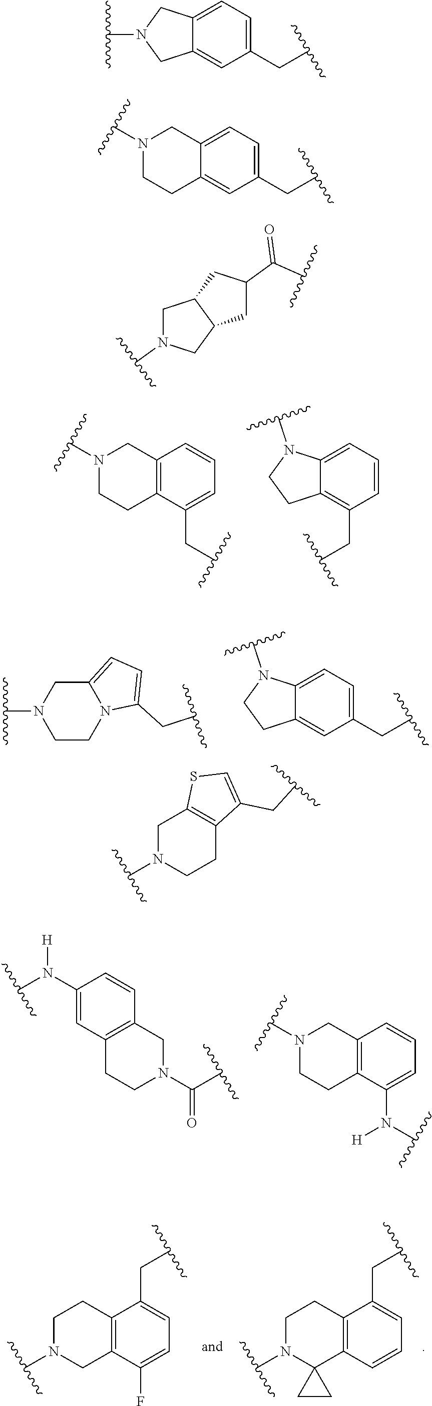

In certain embodiments, the Linker is selected from the group consisting of:

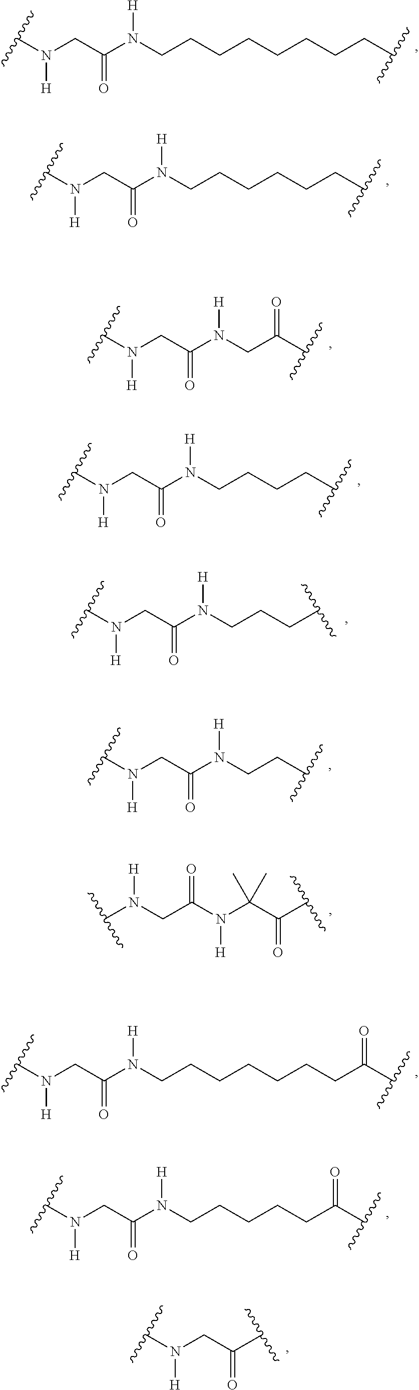

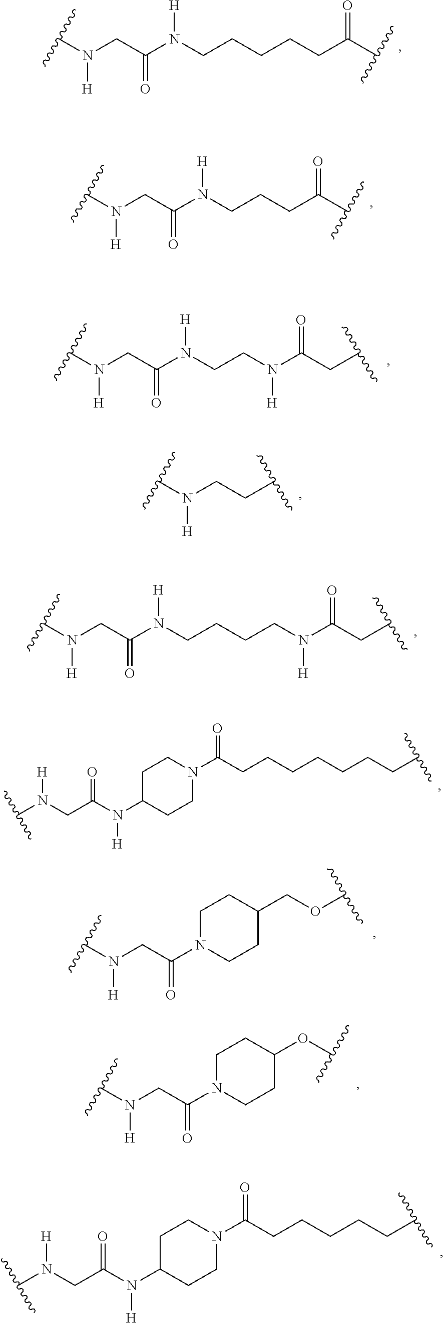

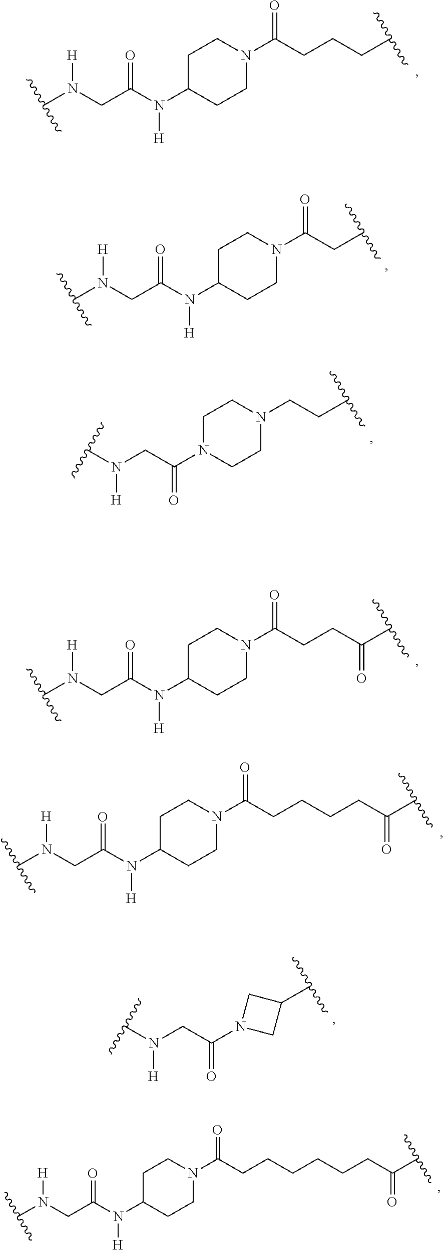

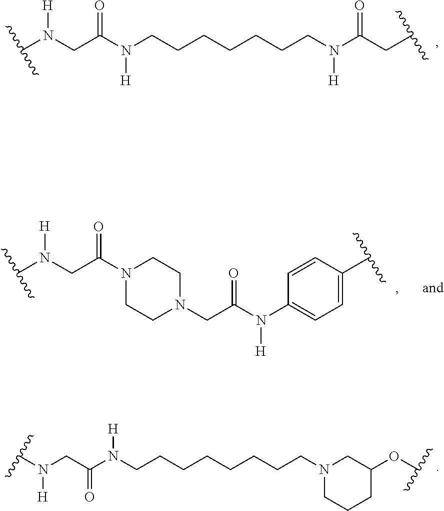 [SMILES: *CC(=O)NCCCCCCCNC(=O)CN* ; *NCC(=O)N1CCN(CC(=O)Nc2ccc(*)cc2)CC1 ; *NCC(=O)NCCCCCCCCN1CCCC(O*)C1]

In certain embodiments, the Linker is selected from the group consisting of:

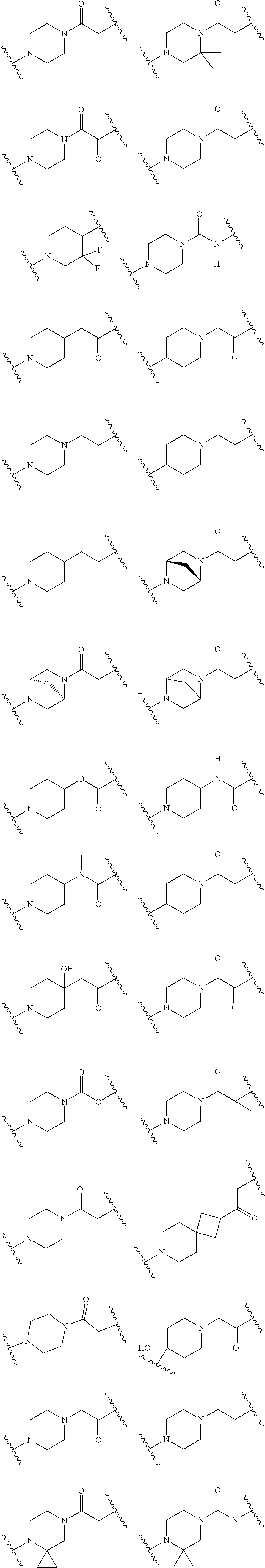

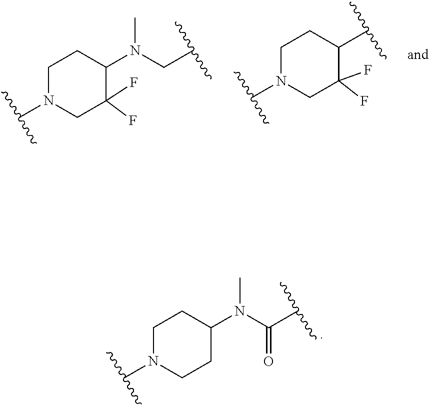 [SMILES: *CN(C)C1CCN(*)CC1(F)F ; *C1CCN(*)CC1(F)F ; *C(=O)N(C)C1CCN(*)CC1]

In certain embodiments, the Linker is selected from the group consisting of:

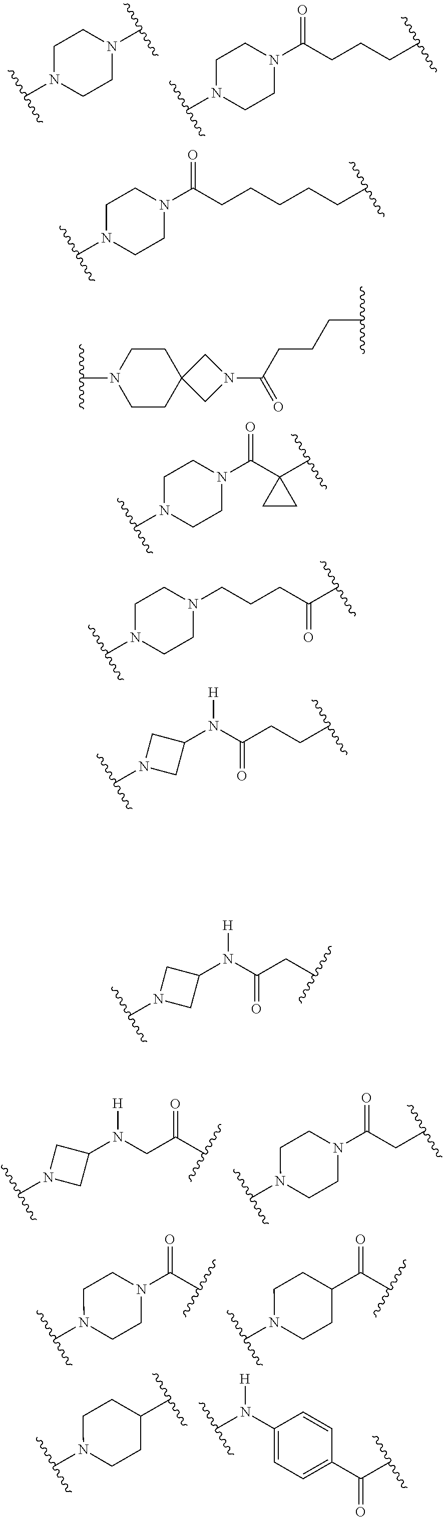

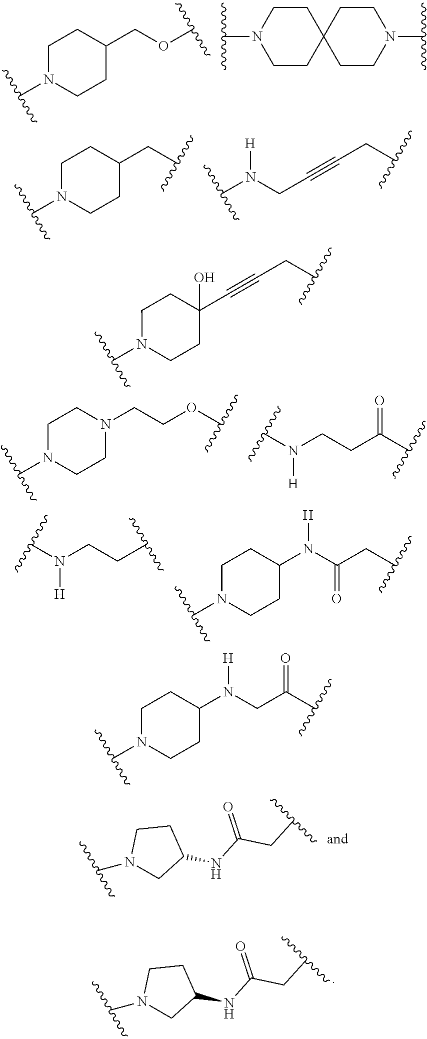

In certain embodiments, the Linker is selected from:

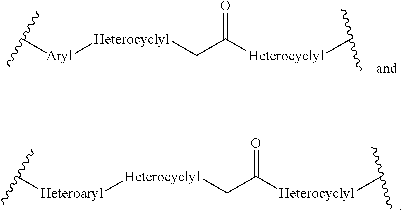

In certain embodiments, the Linker is selected from the group consisting of:

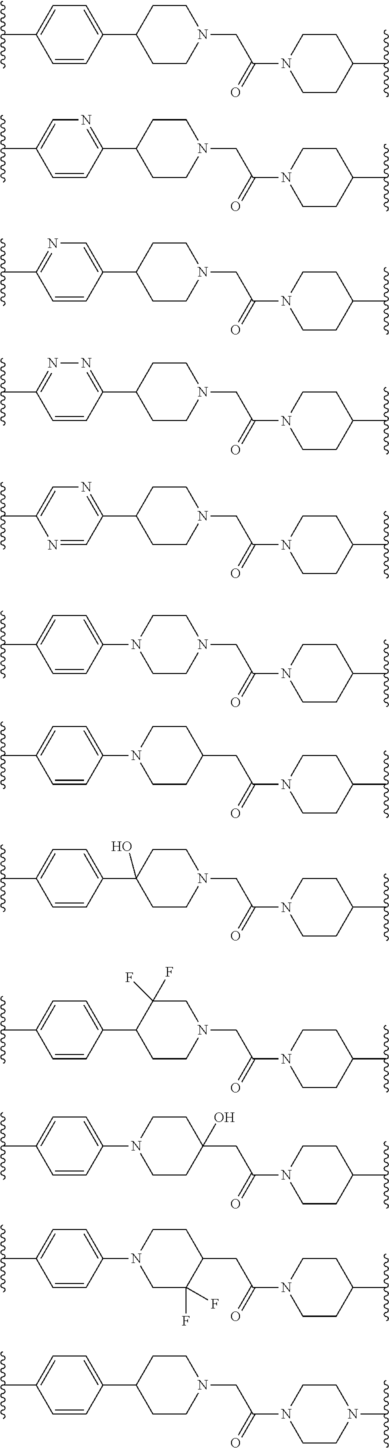

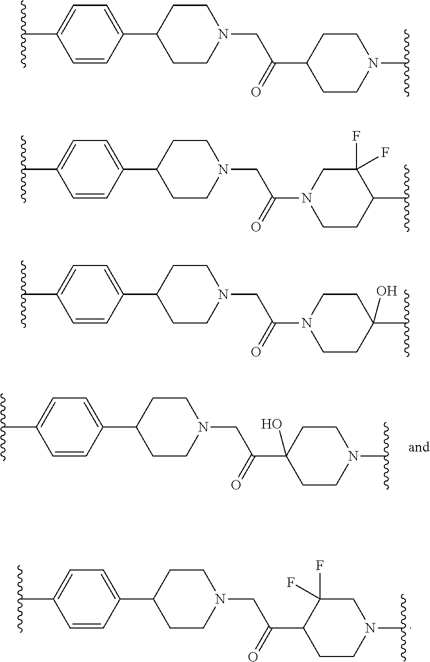

In certain embodiments, the Linker is selected from:

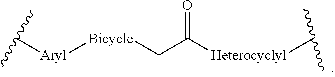

In certain embodiments, the Linker is selected from the group consisting of:

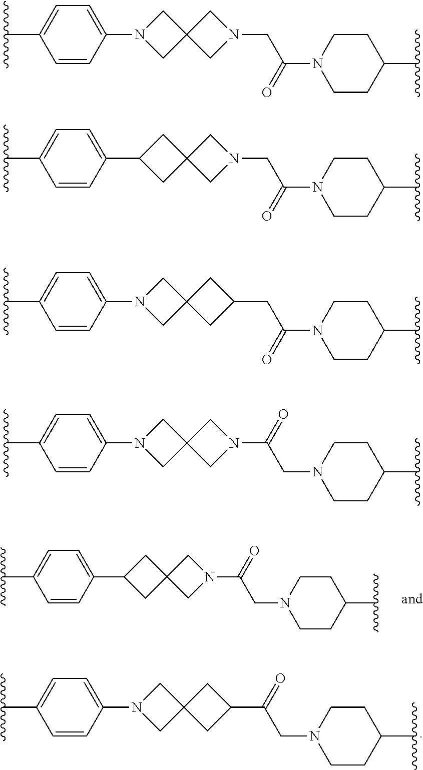 [SMILES: *c1ccc(N2CC3(CN(CC(=O)N4CCC(*)CC4)C3)C2)cc1 ; *c1ccc(C2CC3(C2)CN(CC(=O)N2CCC(*)CC2)C3)cc1 ; *c1ccc(N2CC3(CC(CC(=O)N4CCC(*)CC4)C3)C2)cc1 ; *c1ccc(N2CC3(CN(C(=O)CN4CCC(*)CC4)C3)C2)cc1 ; *c1ccc(C2CC3(C2)CN(C(=O)CN2CCC(*)CC2)C3)cc1 ; *c1ccc(N2CC3(CC(C(=O)CN4CCC(*)CC4)C3)C2)cc1]

In certain embodiments, Linker or a portion thereof is selected from:

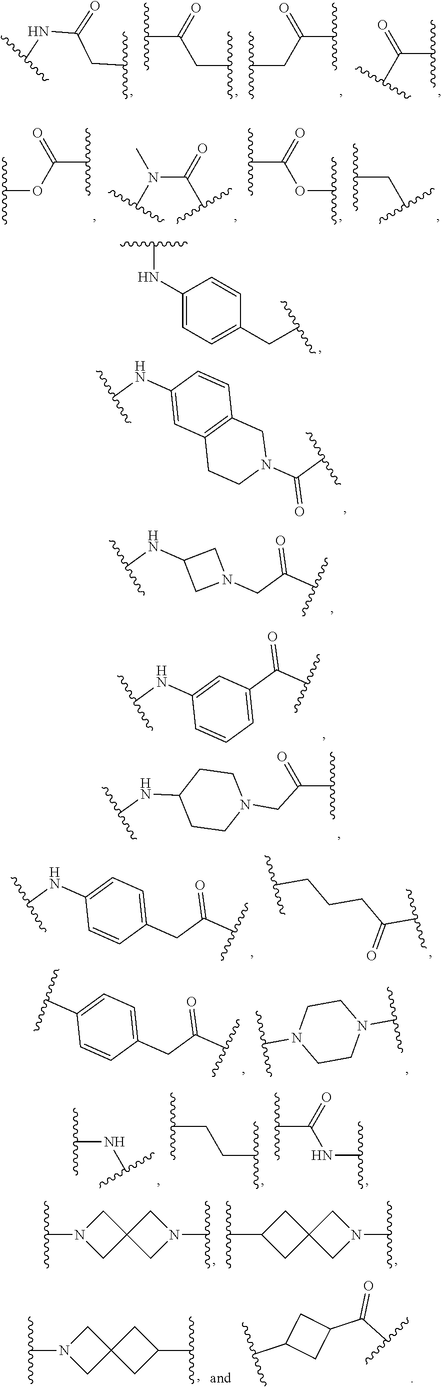

### Embodiments of Compounds of the Invention

In certain embodiments, the compound of the present invention is of Formula:

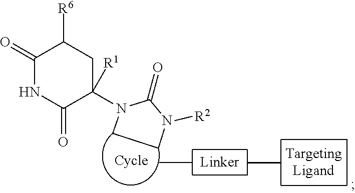

or a pharmaceutically acceptable salt thereof.

In certain embodiments, the compound of the present invention is of Formula:

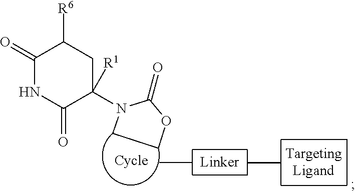

or a pharmaceutically acceptable salt thereof.

In certain embodiments, the compound of the present invention is of Formula:

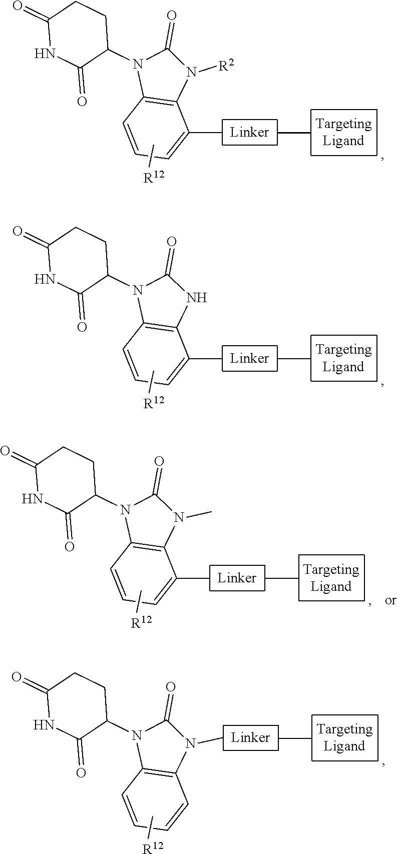

or a pharmaceutically acceptable salt thereof.

In certain embodiments, the compound of the present invention is of Formula:

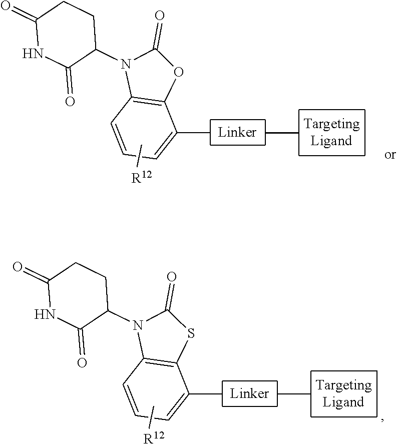

or a pharmaceutically acceptable salt thereof.

In certain embodiments, the compound of the present invention is of Formula:

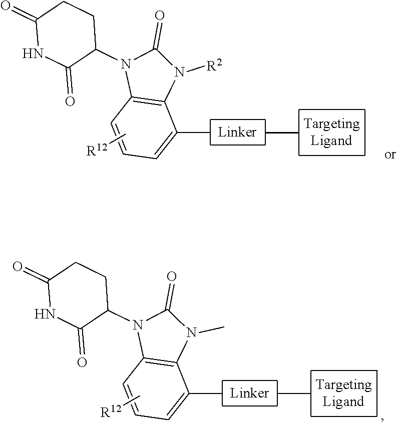

or a pharmaceutically acceptable salt thereof.

In certain embodiments, the compound of the present invention is of Formula:

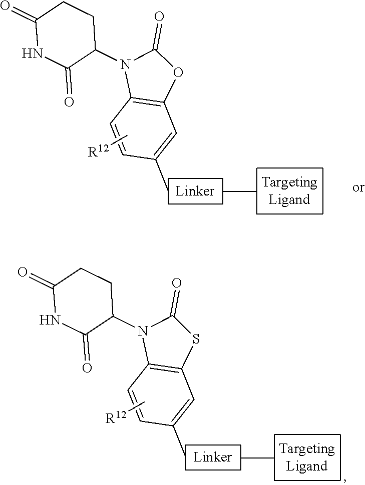

or a pharmaceutically acceptable salt thereof.

In certain embodiments, the compound of the present invention is of Formula:

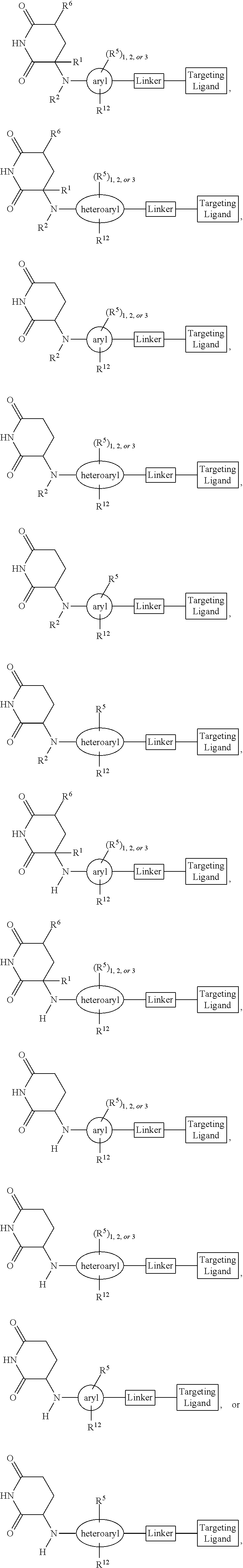

or a pharmaceutically acceptable salt thereof.

In certain embodiments, the compound of the present invention is of Formula:

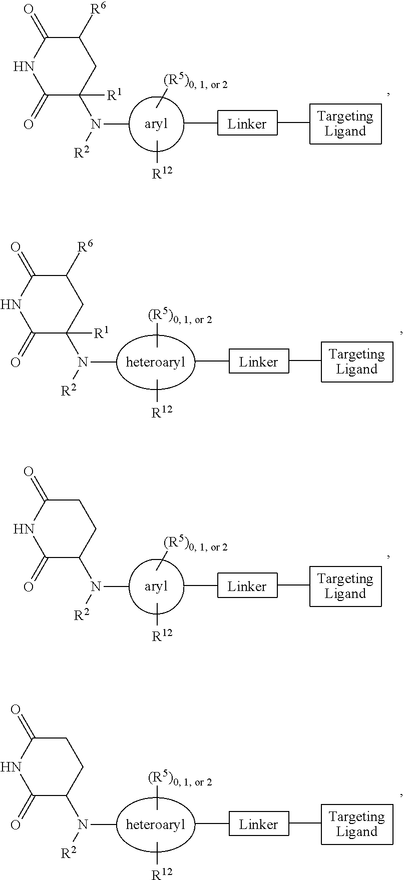

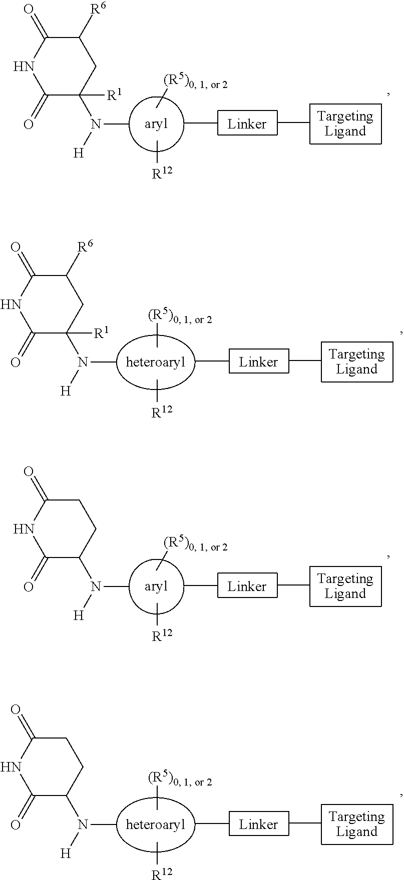

or a pharmaceutically acceptable salt thereof.

In certain embodiments, the compound of the present invention is of Formula:

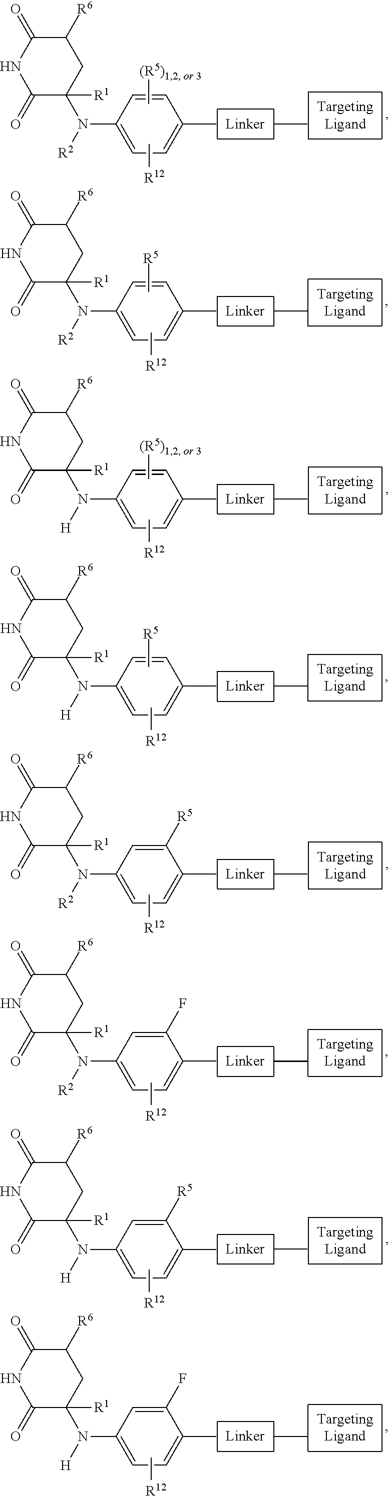

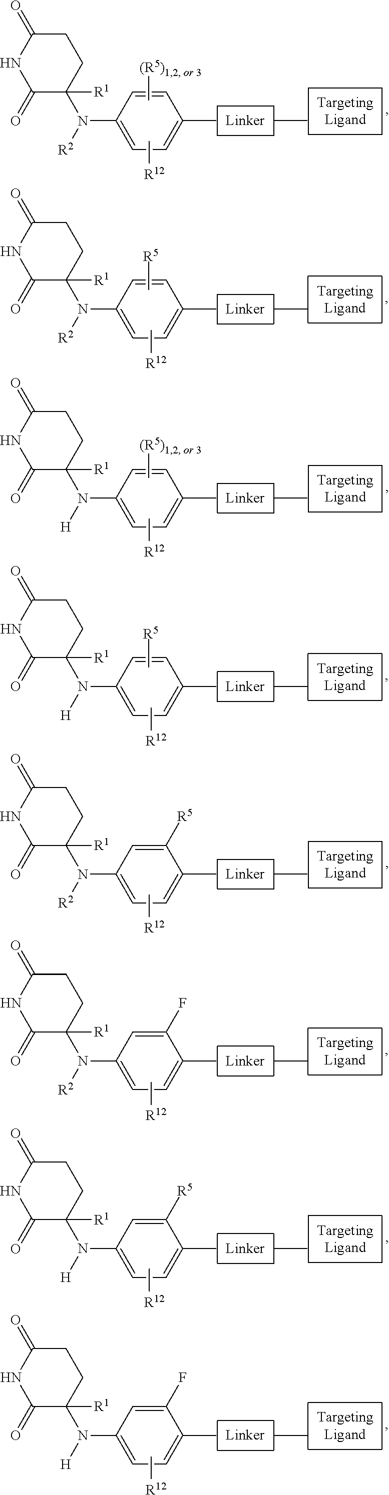

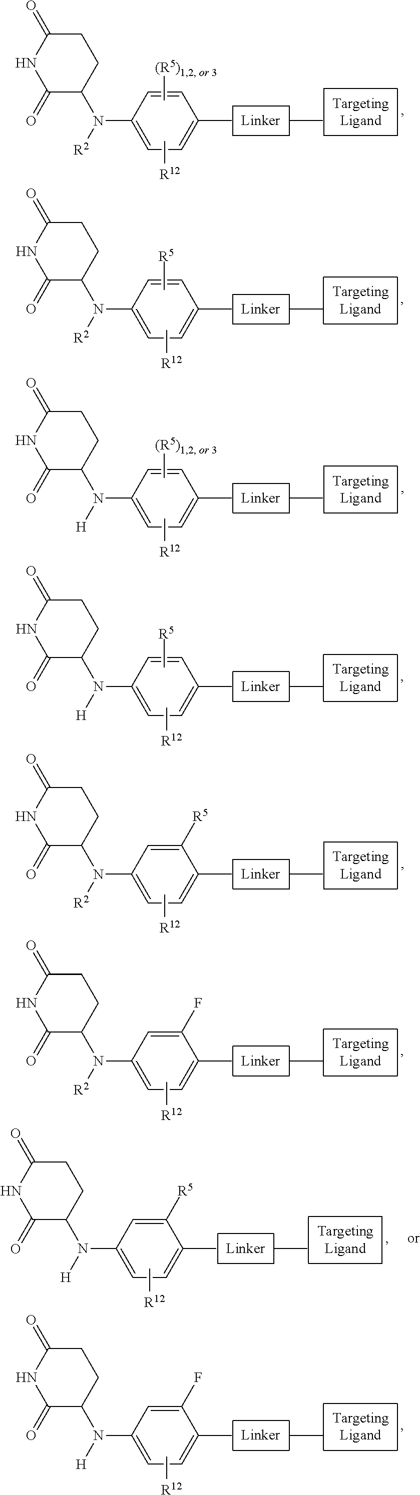

or a pharmaceutically acceptable salt thereof.

In certain embodiments, the compound of the present invention is of Formula:

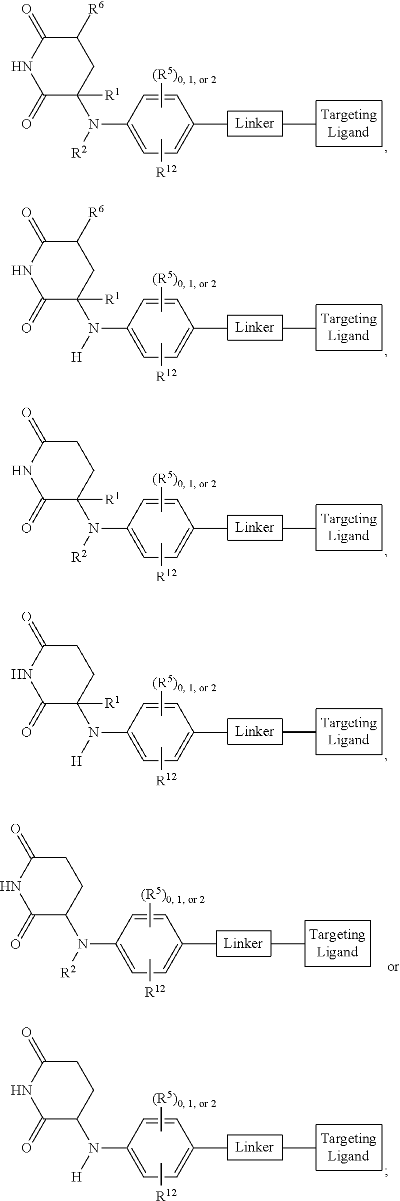

or a pharmaceutically acceptable salt thereof.

In certain embodiments, the compound of the present invention is of Formula:

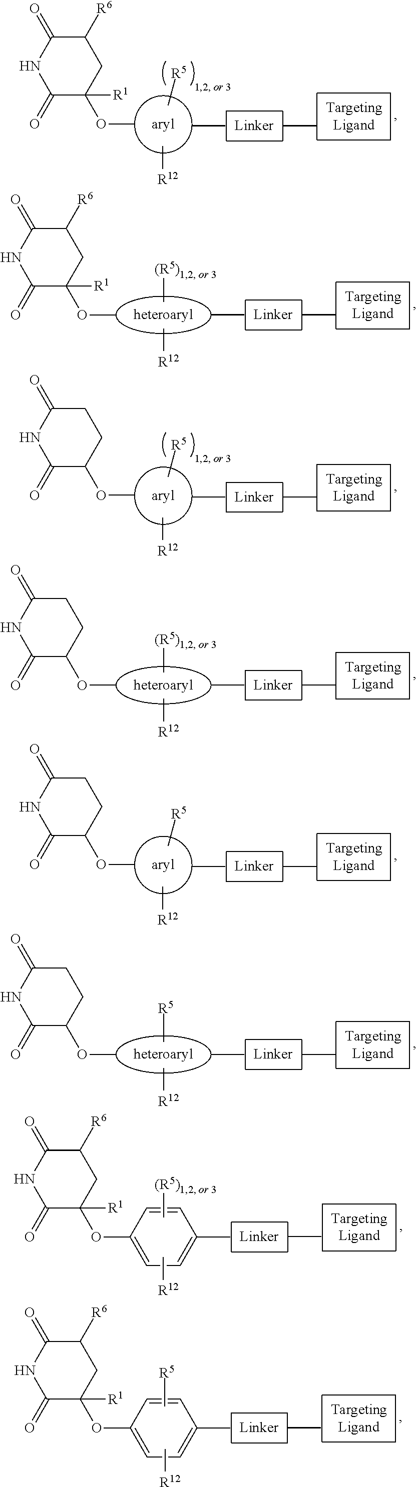

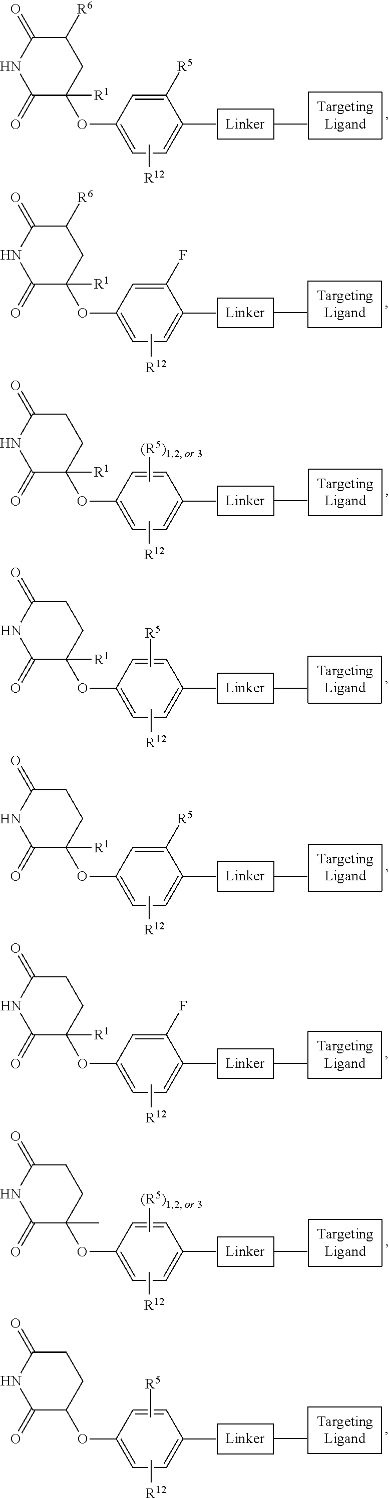

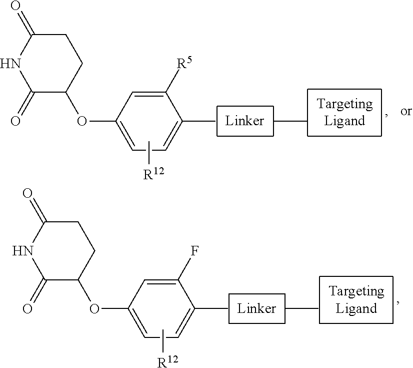

or a pharmaceutically acceptable salt thereof.

In certain embodiments, the compound of the present invention is of Formula:

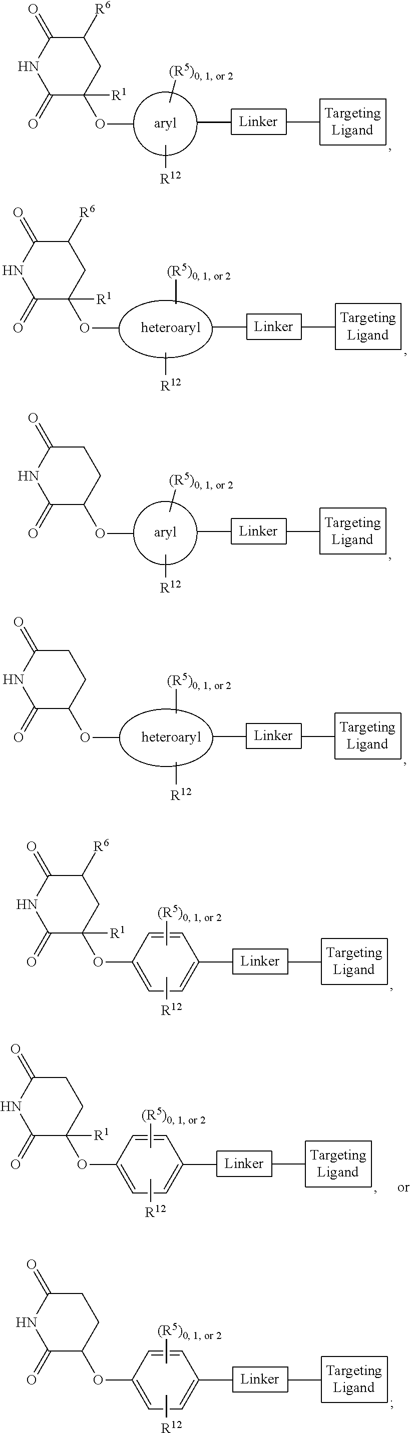

or a pharmaceutically acceptable salt thereof.

In certain embodiments, the compound of the present invention is of Formula:

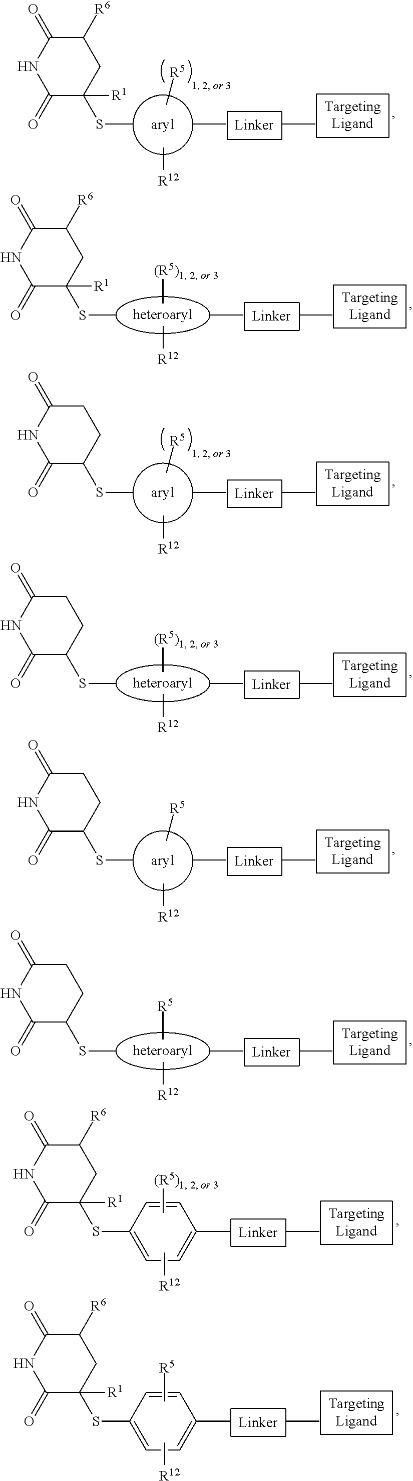

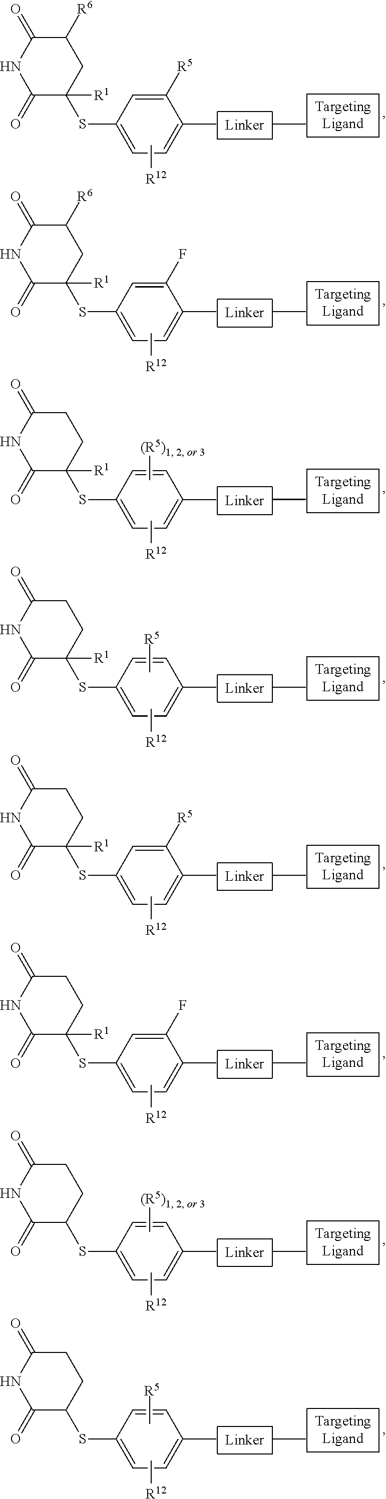

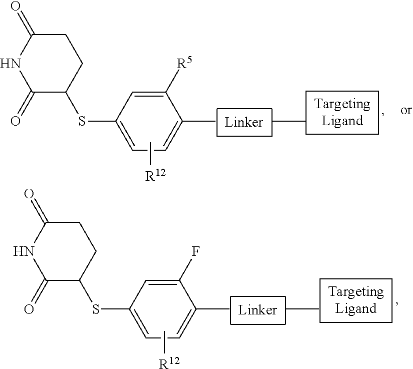

or a pharmaceutically acceptable salt thereof.

In certain embodiments, the compound of the present invention is of Formula:

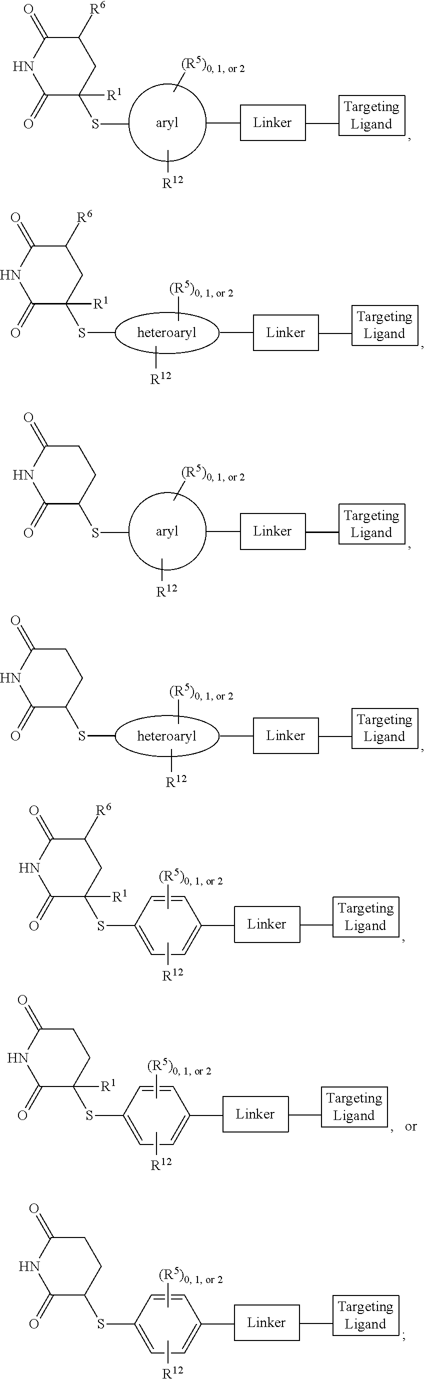

or a pharmaceutically acceptable salt thereof.

In certain embodiments, the compound of the present invention is of Formula:

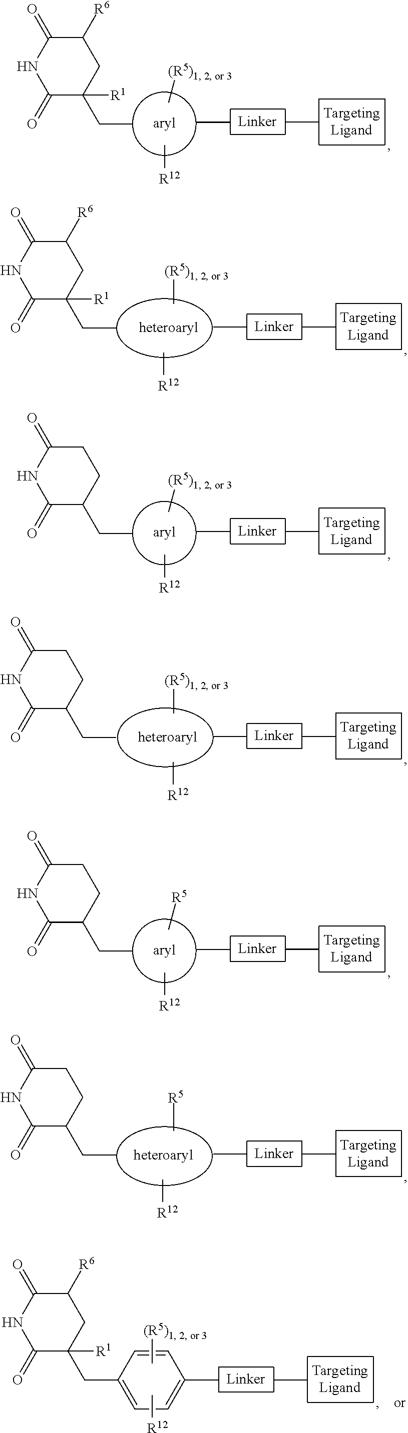

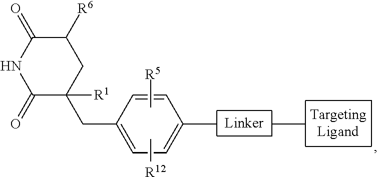

or a pharmaceutically acceptable salt thereof.

In certain embodiments, the compound of the present invention is of Formula:

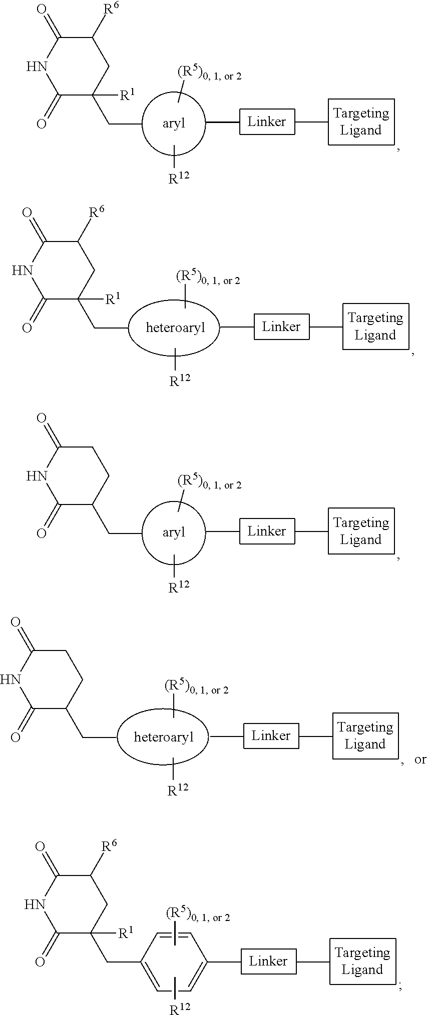

or a pharmaceutically acceptable salt thereof.

In certain embodiments, the compound of the present invention is of Formula:

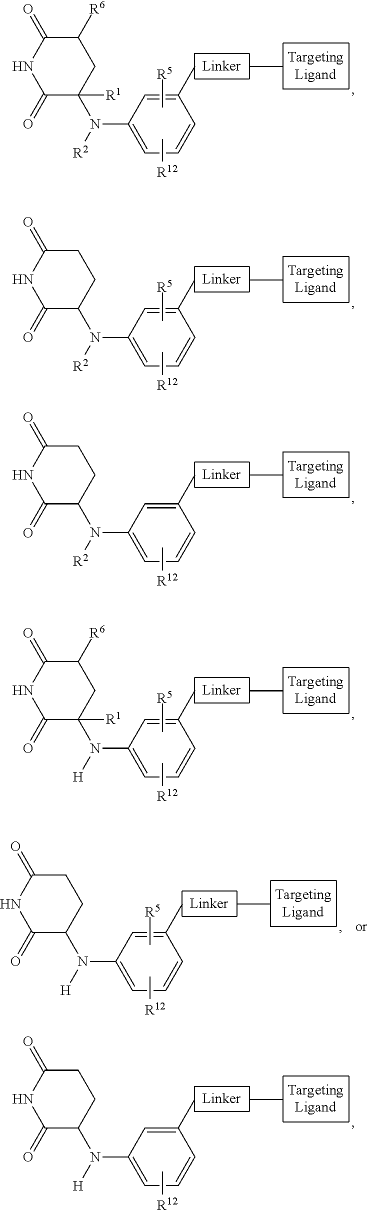

or a pharmaceutically acceptable salt thereof.

In certain embodiments, the compound of the present invention is of Formula:

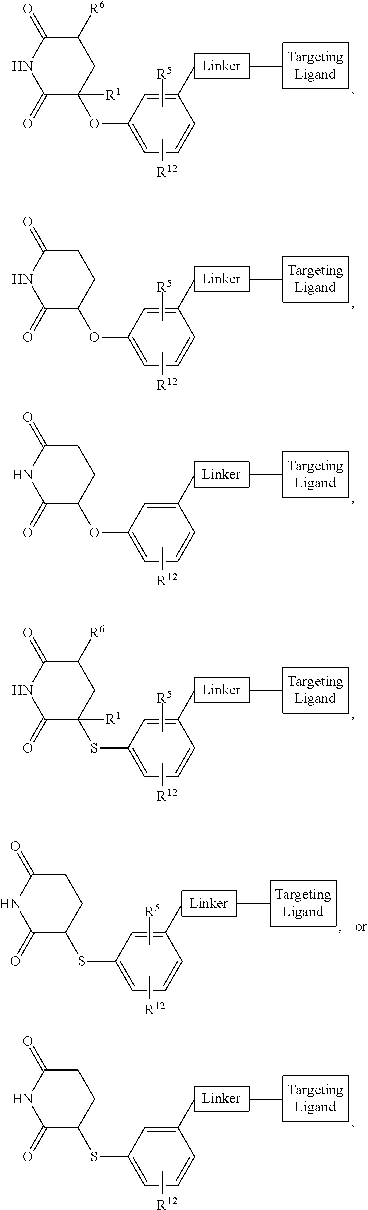

or a pharmaceutically acceptable salt thereof.

In certain embodiments, the compound of the present invention is of Formula:

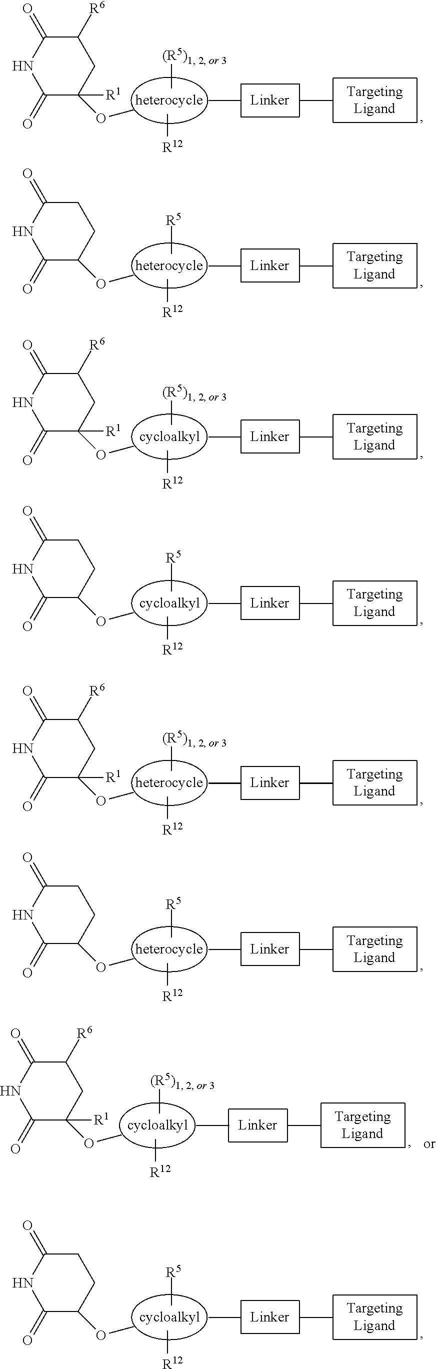

or a pharmaceutically acceptable salt thereof.

In certain embodiments, the compound of the present invention is of Formula:

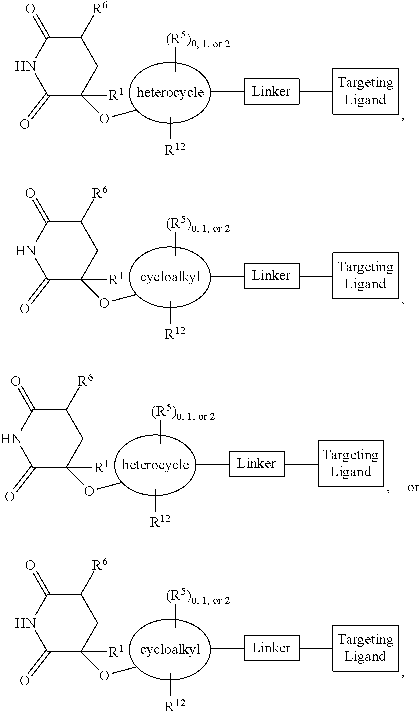

or a pharmaceutically acceptable salt thereof.

In certain embodiments, the compound of the present invention is of Formula:

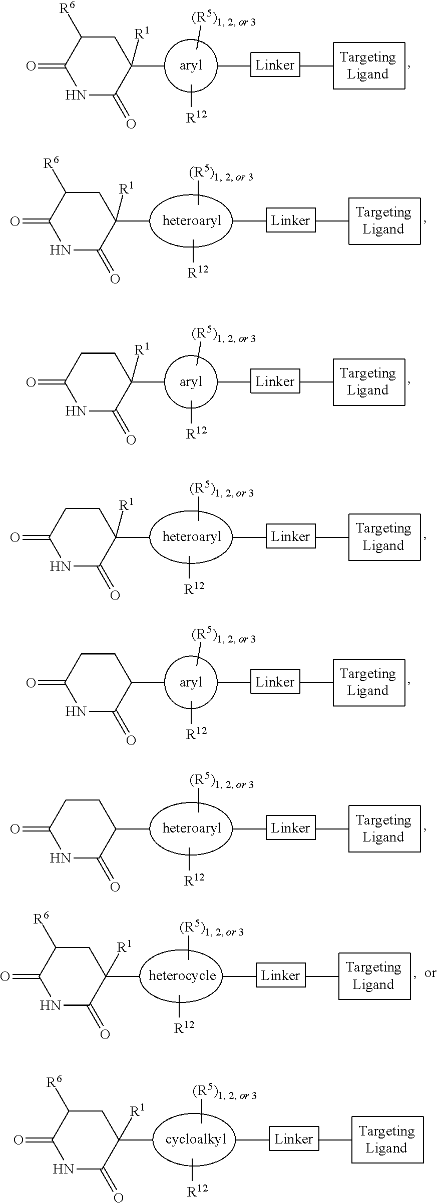

or a pharmaceutically acceptable salt thereof.

In certain embodiments, the compound of the present invention is of Formula:

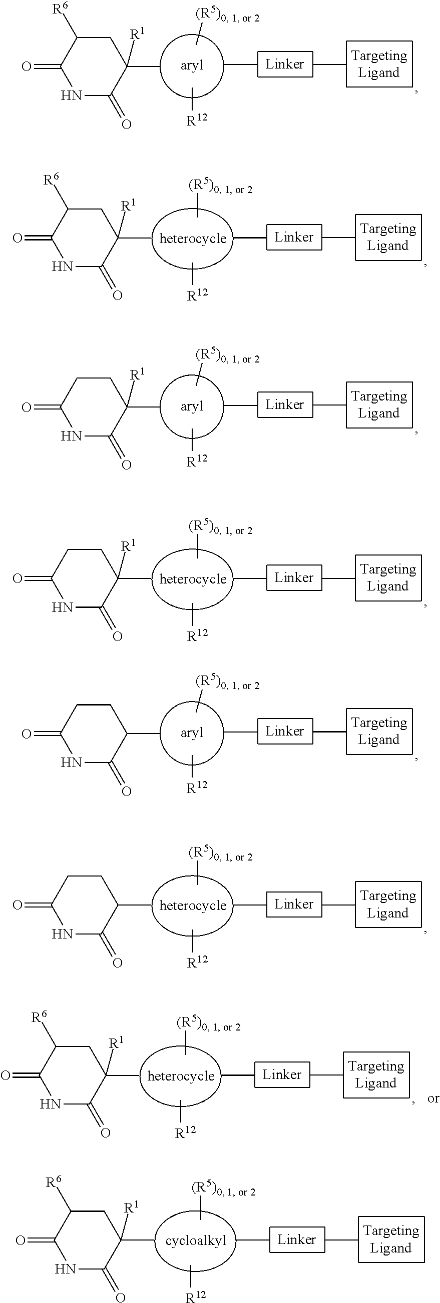

or a pharmaceutically acceptable salt thereof.

In certain embodiments, the compound of the present invention is of Formula:

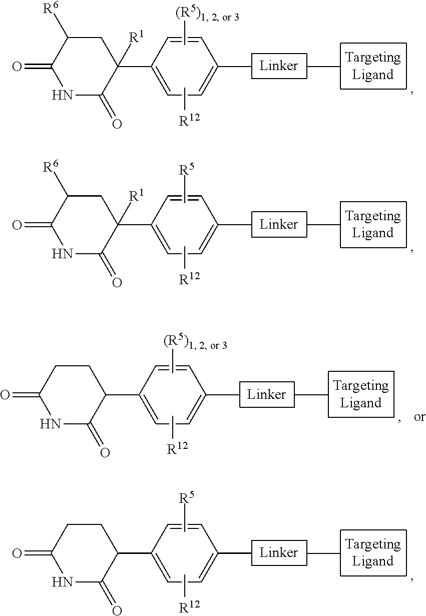

or a pharmaceutically acceptable salt thereof.

In certain embodiments, the compound of the present invention is of Formula:

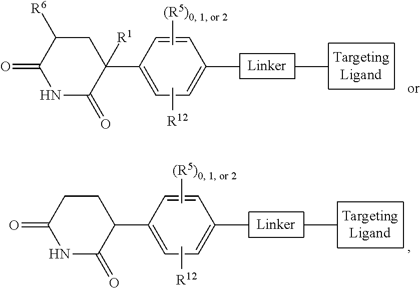

or a pharmaceutically acceptable salt thereof.

In certain embodiments, the compound of the present invention is of Formula:

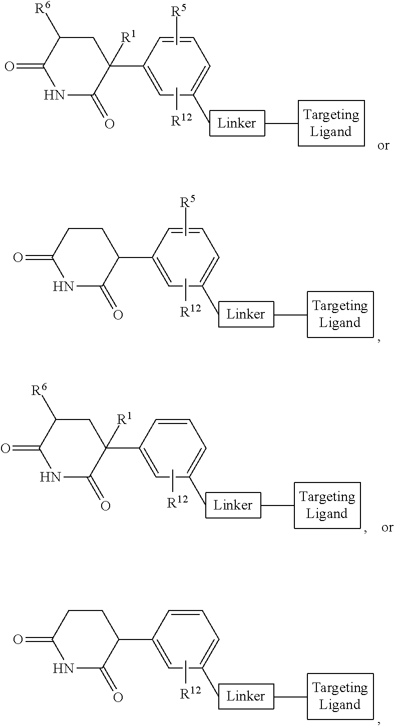

or a pharmaceutically acceptable salt thereof.

In certain embodiments, the compound of the present invention is of Formula:

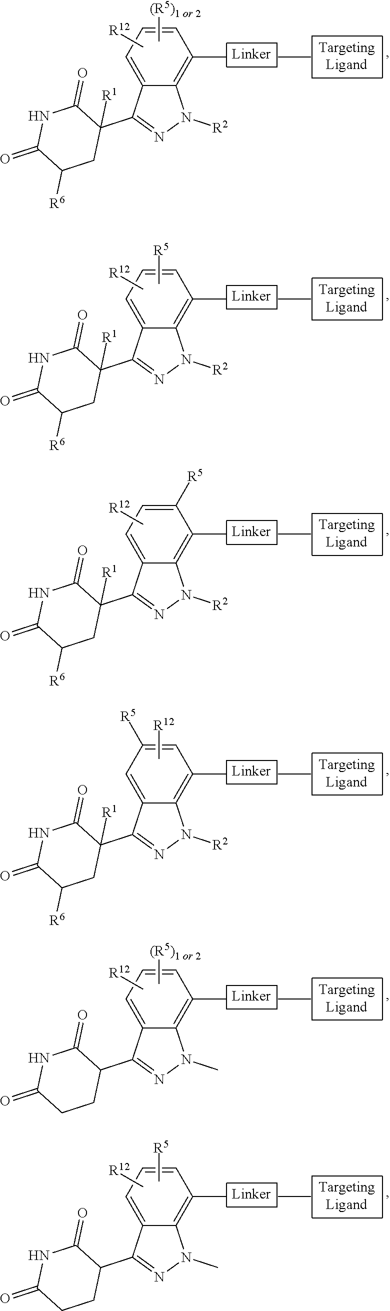

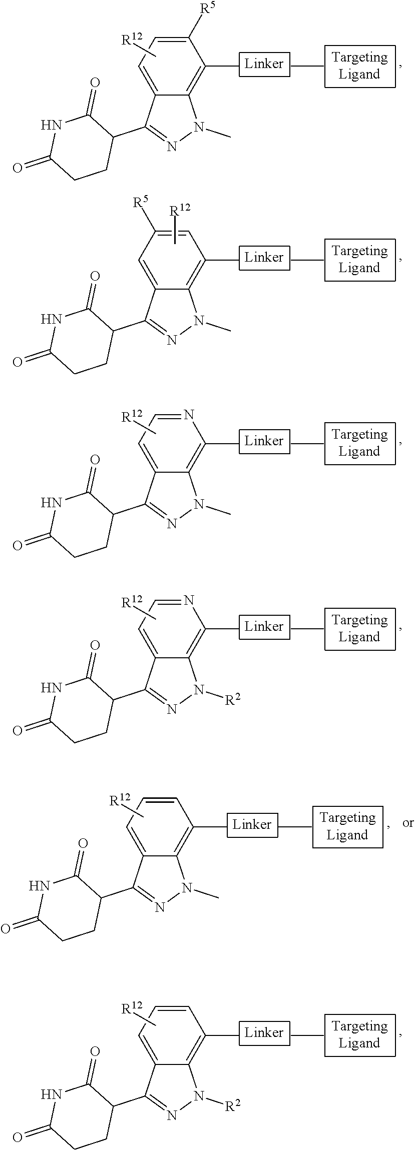

or a pharmaceutically acceptable salt thereof.

In certain embodiments, the compound of the present invention is of Formula.

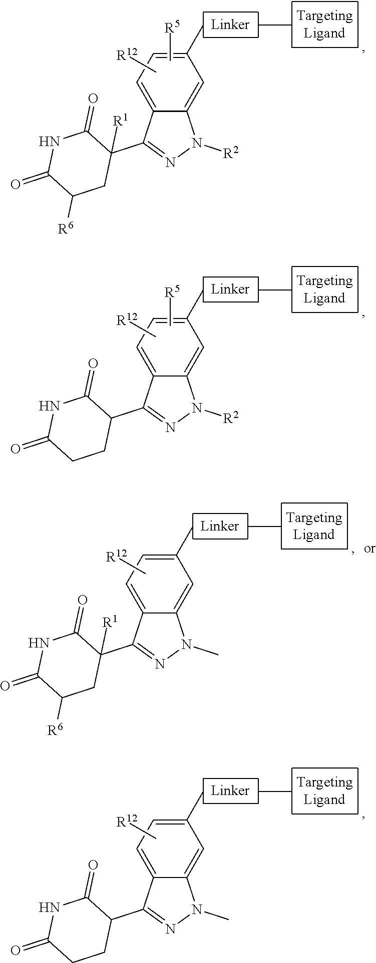

or a pharmaceutically acceptable salt thereof.

In certain embodiments, the compound of the present invention is of Formula:

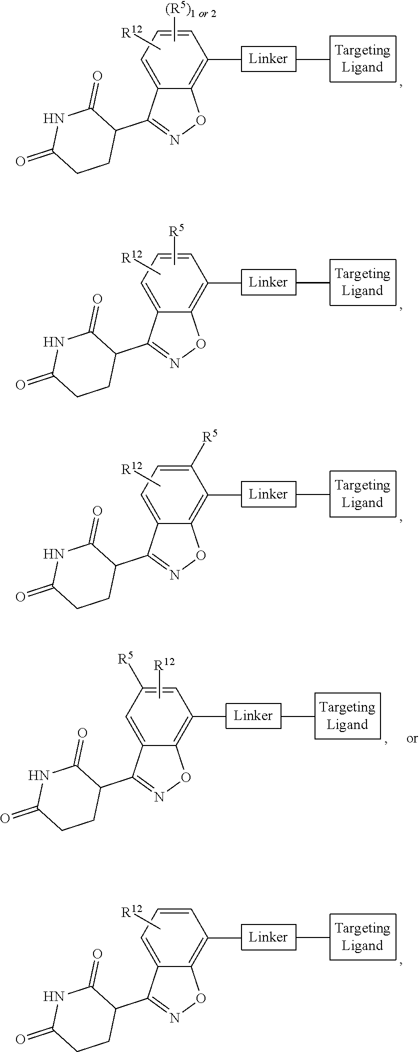

or a pharmaceutically acceptable salt thereof.

In certain embodiments, the compound of the present invention is of Formula:

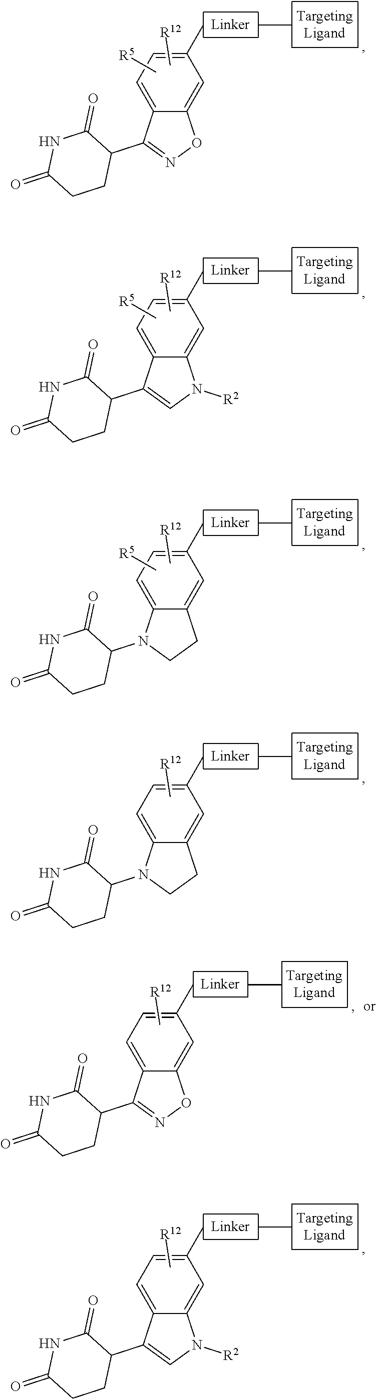

or a pharmaceutically acceptable salt thereof.

In certain embodiments, the compound of the present invention is of Formula:

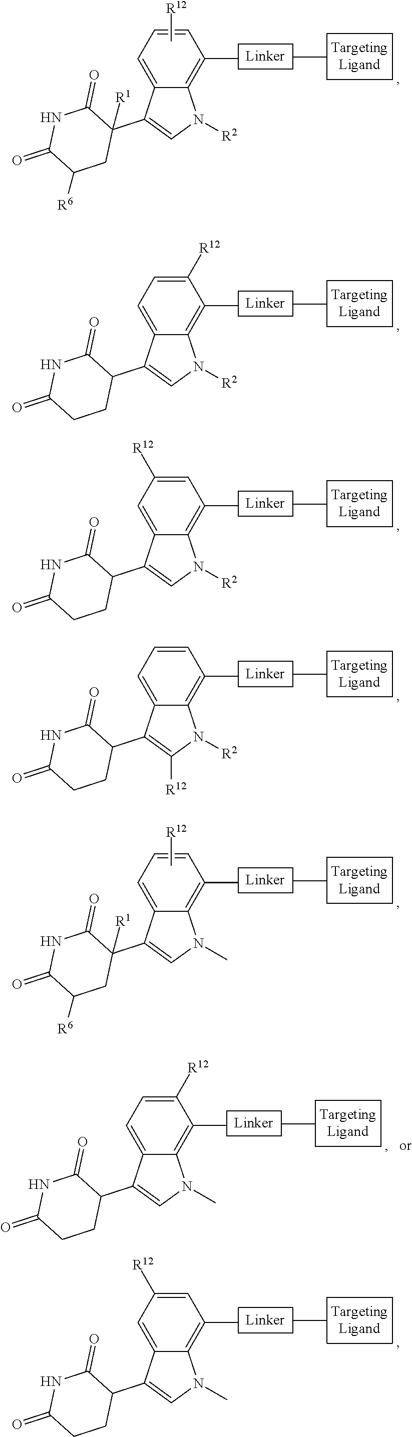

or a pharmaceutically acceptable salt thereof.

In certain embodiments, the compound of the present invention is of Formula:

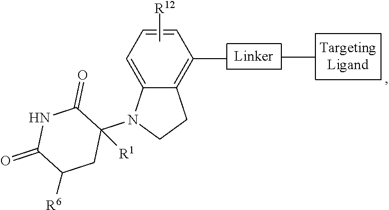

or a pharmaceutically acceptable salt thereof.

In certain embodiments, the compound of the present invention is of Formula:

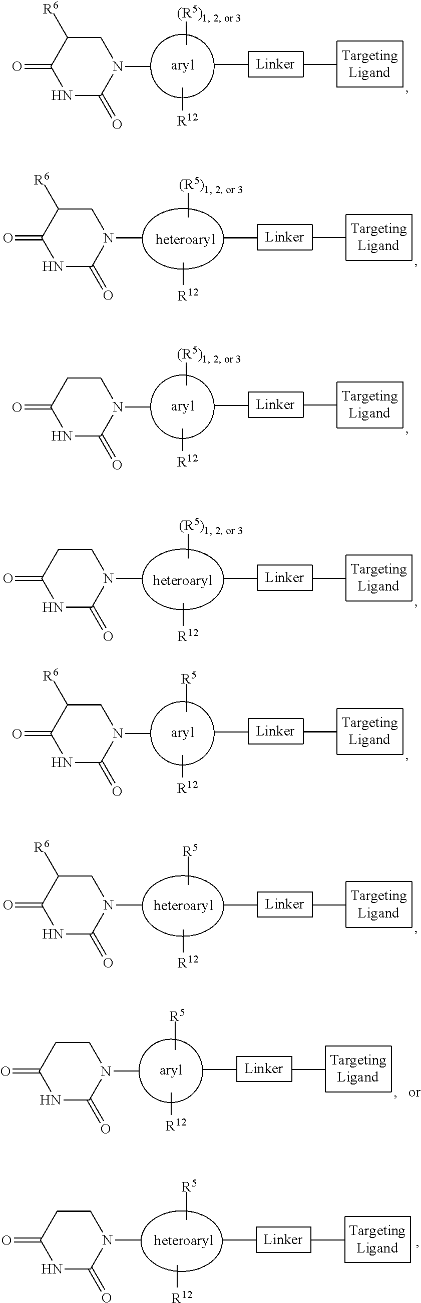

or a pharmaceutically acceptable salt thereof.

In certain embodiments, the compound of the present invention is of Formula:

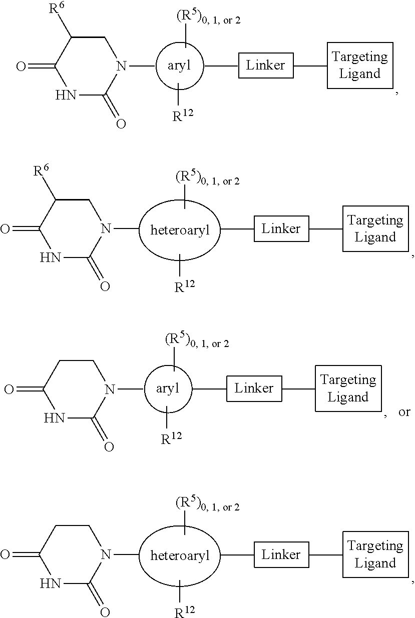

or a pharmaceutically acceptable salt thereof.

In certain embodiments, the compound of the present invention is of Formula:

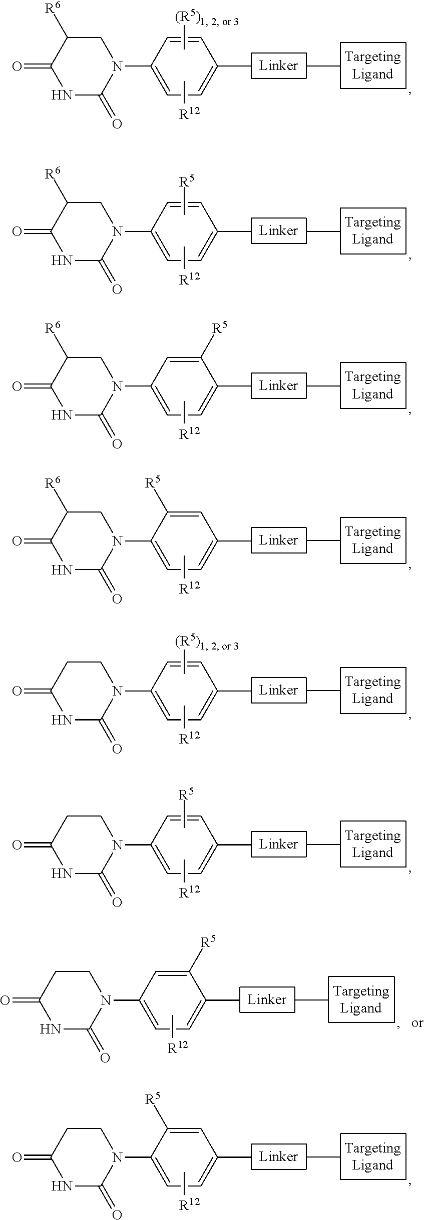

or a pharmaceutically acceptable salt thereof.

In certain embodiments, the compound of the present invention is of Formula:

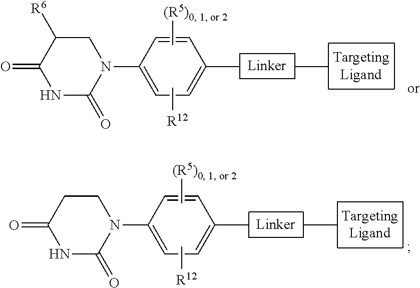

In certain embodiments, the compound of the present invention is of Formula:

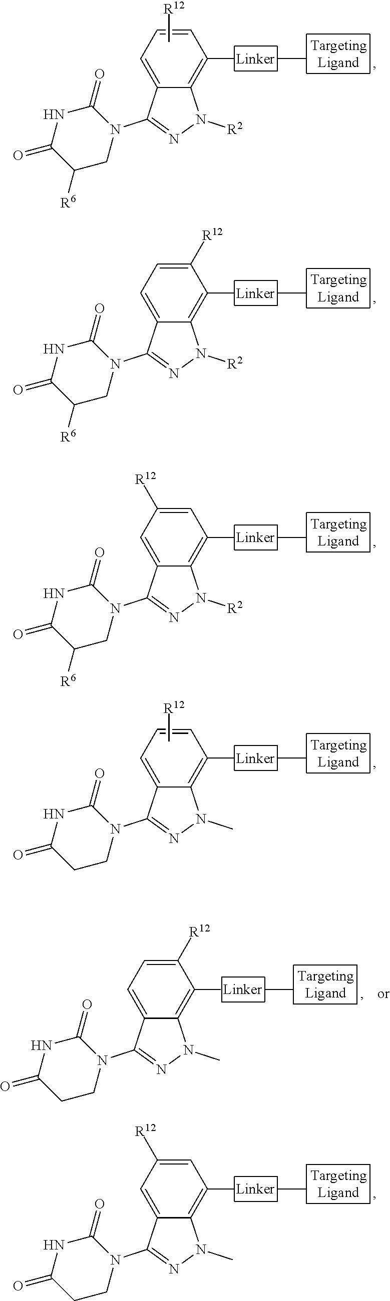

or a pharmaceutically acceptable salt thereof.

In certain embodiments, the compound of the present invention is of Formula:

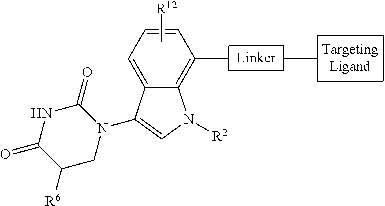

or a pharmaceutically acceptable salt thereof.

In certain embodiments, the compound of the present invention is of Formula:

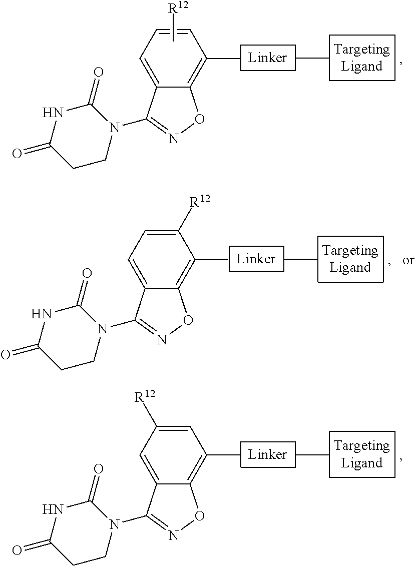

or a pharmaceutically acceptable salt thereof.

In certain embodiments, the compound of the present invention is of Formula:

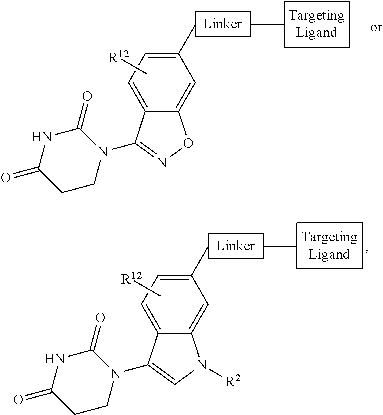

or a pharmaceutically acceptable salt thereof.

In certain embodiments, the compound of the present invention is of Formula:

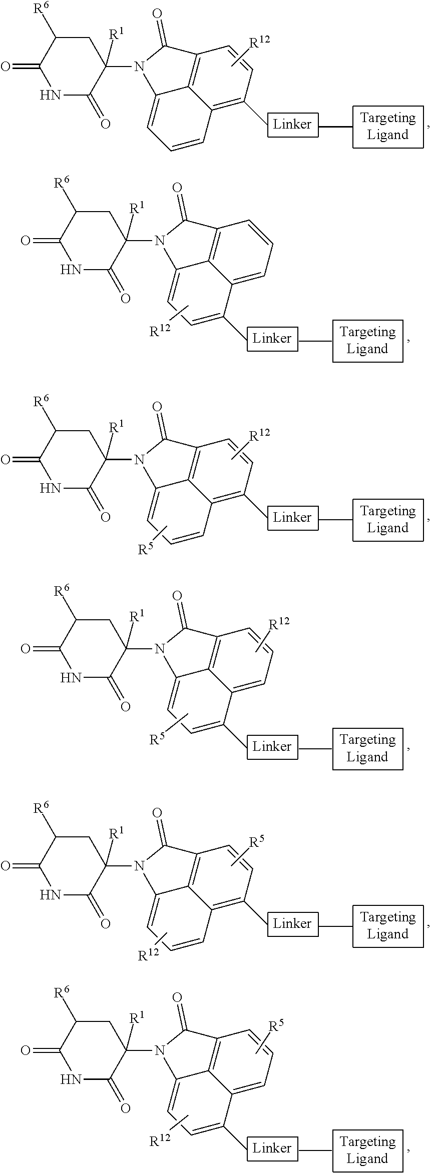

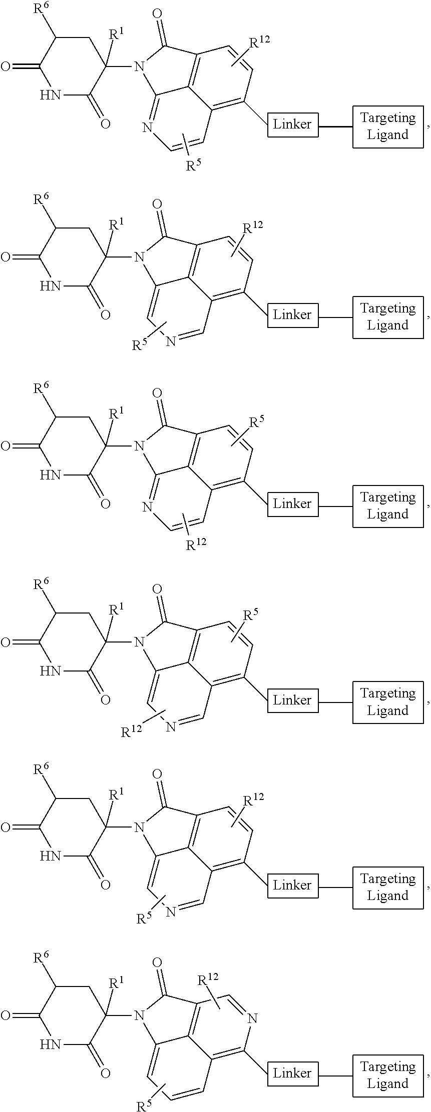

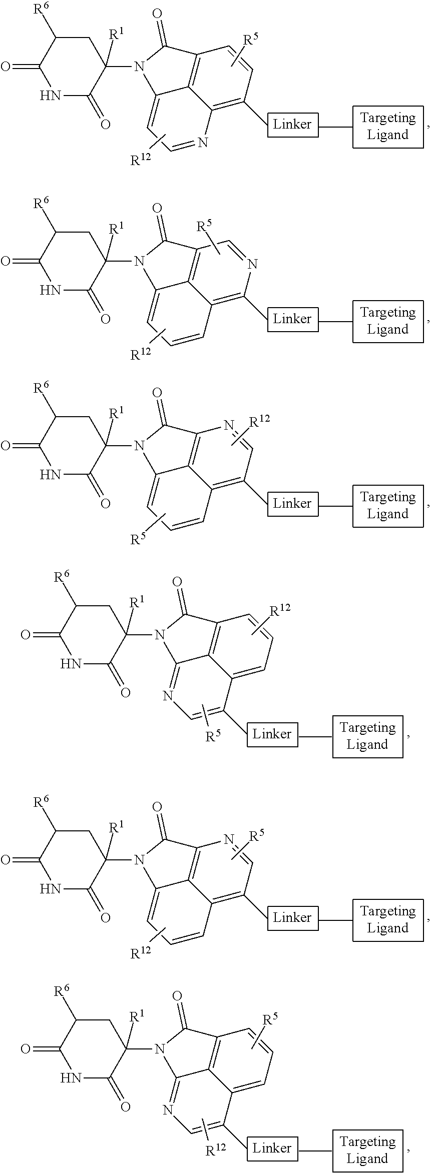

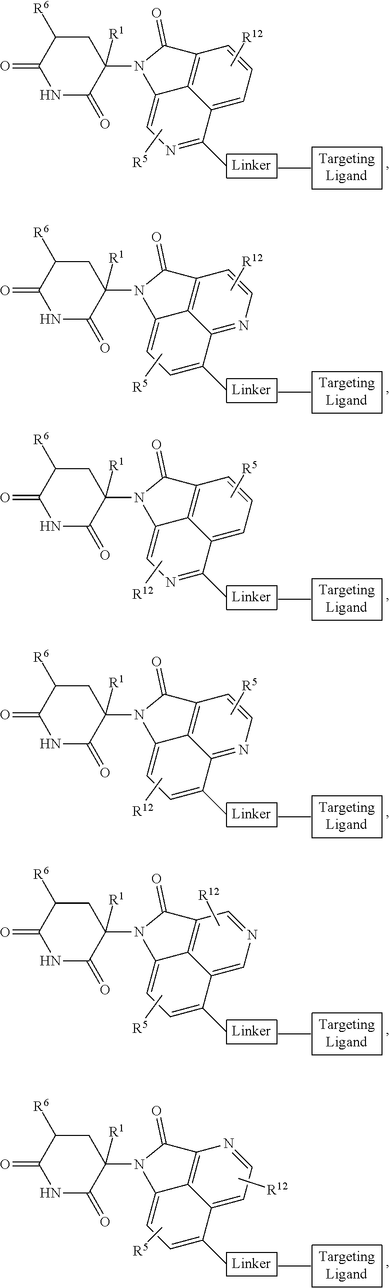

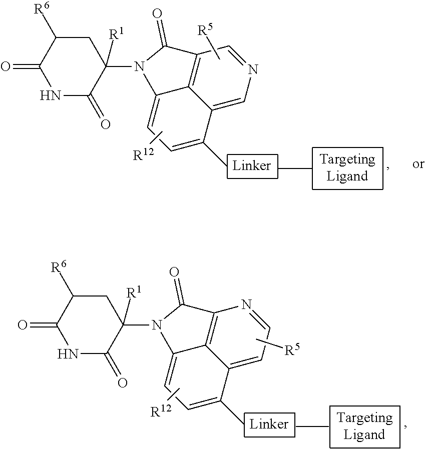

or a pharmaceutically acceptable salt thereof.

### Embodiments of R¹ and R⁶

In certain embodiments, R¹ is alkyl.

In certain embodiments, R¹ is halogen. In certain embodiments, R¹ is halogen, wherein the halogen is F. In certain embodiments, R¹ is halogen, wherein the halogen is Cl. In certain embodiments, R¹ is halogen, wherein the halogen is Br. In certain embodiments, R¹ is halogen, wherein the halogen is I.

In certain embodiments, R⁶ is alkyl.

In alternative embodiments, R⁶ is haloalkyl.

In certain embodiments, R¹ and R⁶ are combined to form a single carbon bridge.

In certain embodiments, R¹ and R⁶ are both hydrogen.

### Embodiments of R^{1a}

In certain embodiments, R^{1a} is hydrogen.

In certain embodiments, R^{1a} is C₃-C₈ alkyl.

In certain embodiments, R^{1a} is halogen. In certain embodiments, R^{1a} is halogen, wherein the halogen is F. In certain embodiments, R^{1a} is halogen, wherein the halogen is Cl. In certain embodiments, R^{1a} is halogen, wherein the halogen is Br. In certain embodiments, R^{1a} is halogen, wherein the halogen is I.

In certain embodiments, R^{1a} is alkenyl.

In certain embodiments, R^{1a} is alkynyl.

In certain embodiments, R^{1a} and R⁶ are combined to form a single carbon bridge.

In certain embodiments, R^{1a} and R⁶ are combined to form a two carbon bridge.

### Embodiments of R²

In certain embodiments, R² is hydrogen.

In certain embodiments, R² is alkyl.

In certain embodiments, R is haloalkyl.

In certain embodiments, R² is alkenyl.

In certain embodiments, R² is alkynyl.

In certain embodiments, R² is aryl. In certain embodiments, R² is aryl, wherein the aryl is substituted with 1 or 2 substituents independently selected from R¹⁰. In certain embodiments, R² is phenyl. In certain embodiments, R² is phenyl substituted with 1 or 2 substituents independently selected from R¹⁰.

In certain embodiments, R² is heteroaryl. In certain embodiments, R² is heteroaryl, wherein the heteroaryl is substituted with 1 or 2 substituents independently selected from R¹⁰.

In certain embodiments, R² is heterocycle. In certain embodiments, R² is heterocycle, wherein the heterocycle is substituted with 1 or 2 substituents independently selected from R¹⁰.

In certain embodiments, R² is C(O)R⁹. In certain embodiments, R² is C(O)R⁹, wherein C(O)R⁹ is substituted with 1 or 2 substituents independently selected from R¹⁰.

In certain embodiments, Ri, R², and R⁶ are each hydrogen.

### Embodiments of R⁵

In certain embodiments, R⁵ is hydrogen.

In certain embodiments, each R⁵ is selected from alkyl, haloalkyl, and halogen.

In certain embodiments, R⁵ is alkyl.

In certain embodiments, R⁵ is haloalkyl.

In certain embodiments, R⁵ is alkenyl.

In certain embodiments, R⁵ is alkynyl.

In certain embodiments, R⁵ is halogen. In certain embodiments, R⁵ is halogen, wherein the halogen is F. In certain embodiments, R⁵ is halogen, wherein the halogen is Cl. In certain embodiments, R⁵ is halogen, wherein the halogen is Br. In certain embodiments, R⁵ is halogen, wherein the halogen is I.

In certain embodiments, R⁵ is heteroaryl. In certain embodiments, R⁵ is aryl. In certain embodiments, R⁵ is heterocycle.

In certain embodiments, R⁵ is cyano.

In certain embodiments, R⁵ is —NR⁷R⁸. In certain embodiments, R⁵ is —NR⁷C(O)R⁹. In certain embodiments, R⁵ is —NR⁷C(S)R⁹. In certain embodiments, R⁵ is —NR⁷C(O)R⁹. In certain embodiments, R⁵ is —NR⁷S(O)₂R⁹.

In certain embodiments, R⁵ is —OR⁷.

In certain embodiments, R⁵ is —SR⁷. In certain embodiments, R⁵ is —S(O)₂R⁹.

In certain embodiments, R⁵ is —C(O)R⁹.

### Embodiments of R⁷ and R⁸

In certain embodiments, R⁷ is hydrogen.

In certain embodiments, R⁷ is alkyl.

In certain embodiments, R⁷ is methyl.

In certain embodiments, R⁷ is haloalkyl.

In certain embodiments, R⁷ is CF₃.

In certain embodiments, R⁷ is aryl. In certain embodiments, R⁷ is aryl optionally substituted with 1, 2, or 3 substituents independently selected from R^{10b}.

In certain embodiments, R⁷ is heteroaryl. In certain embodiments, R⁷ is heteroaryl optionally substituted with 1, 2, or 3 substituents independently selected from R^{10b}.

In certain embodiments, R⁷ is heterocycle. In certain embodiments, R⁷ is heterocycle optionally substituted with 1, 2, or 3 substituents independently selected from R^{10b}.

In certain embodiments, R⁷ is C(O)R¹⁴.

In certain embodiments, R⁷ is C(O) alkyl.

In certain embodiments, R is hydrogen.

In certain embodiments, R is alkyl.

In certain embodiments, R is methyl.

In certain embodiments, R is haloalkyl.

In certain embodiments, R is CF₃.

In certain embodiments, R⁷ and R⁸ are both hydrogen.

### Embodiments of R⁹

In certain embodiments, R⁹ is hydrogen.

In certain embodiments, R⁹ is alkyl.

In certain embodiments, R⁹ is methyl.

In certain embodiments, R⁹ is ethyl.

In certain embodiments, R⁹ is haloalkyl.

In certain embodiments, R⁹ is CF₃.

In certain embodiments, R⁹ is aryl. In certain embodiments, R⁹ is aryl optionally substituted with 1, 2, or 3 substituents independently selected from R¹⁰.

In certain embodiments, R⁹ is heteroaryl. In certain embodiments, R⁹ is heteroaryl optionally substituted with 1, 2, or 3 substituents independently selected from R¹⁰.

In certain embodiments, R⁹ is heterocycle. In certain embodiments, R⁹ is heterocycle optionally substituted with 1, 2, or 3 substituents independently selected from R¹⁰.

In certain embodiments, R⁹ is —NR⁷R.

In certain embodiments, R⁹ is —NH₂.

In certain embodiments, R⁹ is —N(CH₃)H.

In certain embodiments, R⁹ is —N(CH₃)₂.

In certain embodiments, R⁹ is —OR⁷.

In certain embodiments, R⁹ is —OH.

In certain embodiments, R⁹ is —SR⁷.

In certain embodiments, R⁹ is —SH.

### Embodiments of R¹⁰

In certain embodiments, R¹⁰ is hydrogen.

In certain embodiments, R¹⁰ is alkyl.

In certain embodiments, R¹⁰ is methyl.

In certain embodiments, R¹⁰ is ethyl.

In certain embodiments, R¹⁰ is haloalkyl.

In certain embodiments, R¹⁰ is CF₃.

In certain embodiments, R¹⁰ is aryl. In certain embodiments, R¹⁰ is aryl optionally substituted with 1, 2, or 3 substituents independently selected from R⁵.

In certain embodiments, R¹⁰ is heteroaryl. In certain embodiments, R¹⁰ is heteroaryl optionally substituted with 1, 2, or 3 substituents independently selected from R⁵.

In certain embodiments, R¹⁰ is heterocycle. In certain embodiments, R¹⁰ is heterocycle optionally substituted with 1, 2, or 3 substituents independently selected from R⁵.

In certain embodiments, R¹⁰ is —NR¹¹R¹³

In certain embodiments, R¹⁰ is —NH₂.

In certain embodiments, R¹⁰ is —N(CH₃)H.

In certain embodiments, R¹⁰ is —N(CH₃)2.

In certain embodiments, R¹⁰ is —OR″.

In certain embodiments, R¹⁰ is —OH.

In certain embodiments, R¹⁰ is —SR¹¹.

In certain embodiments, R¹⁰ is —SH.

In certain embodiments, R¹⁰ is alkenyl.

In certain embodiments, R¹⁰ is alkynyl.

In certain embodiments, R¹⁰ is cyano.

In certain embodiments, R¹⁰ is nitro.

In certain embodiments, R¹⁰ is —C(O)R¹⁴.

In certain embodiments, R¹⁰ is —C(O)alkyl.

In certain embodiments, R¹⁰ is —C(O)N(alkyl)₂.

In certain embodiments, R¹⁰ is —C(O)N(H)(alkyl).

In certain embodiments, R¹⁰ is —C(S)R¹⁴.

In certain embodiments, R¹⁰ is —C(S)alkyl.

In certain embodiments, R¹⁰ is —C(S)N(alkyl)₂.

In certain embodiments, R¹⁰ is —C(S)N(H)(alkyl).

In certain embodiments, R¹⁰ is —S(O)R¹⁴.

In certain embodiments, R¹⁰ is —S(O)₂R¹⁴.

In certain embodiments, R¹⁰ is —P(O)(R¹⁴)₂.

### Embodiments of R¹¹ and R¹³

In certain embodiments, R¹¹ is hydrogen.

In certain embodiments, R¹¹ is alkyl.

In certain embodiments, R¹¹ is methyl.

In certain embodiments, R¹¹ is haloalkyl.

In certain embodiments, R¹¹ is CF₃.

In certain embodiments, R¹¹ is aryl. In certain embodiments, R¹¹ is aryl optionally substituted with 1, 2, or 3 substituents independently selected from R⁵.

In certain embodiments, R¹¹ is aryl optionally substituted with 1 or 2 substituents independently selected from R⁵.

In certain embodiments, R¹¹ is heteroaryl. In certain embodiments, R¹¹ is heteroaryl optionally substituted with 1, 2, or 3 substituents independently selected from R⁵.

In certain embodiments, R¹¹ is heteroaryl optionally substituted with 1 or 2 substituents independently selected from R⁵.

In certain embodiments, R¹¹ is heterocycle. In certain embodiments, R¹¹ is heterocycle 30 optionally substituted with 1, 2, or 3 substituents independently selected from R⁵.

In certain embodiments, R¹¹ is heterocycle optionally substituted with 1 or 2 substituents independently selected from R⁵.

In certain embodiments, R¹¹ is C(O)R¹⁴.

In certain embodiments, R¹¹ is C(O)alkyl.

In certain embodiments, R¹³ is hydrogen.

In certain embodiments, R¹³ is alkyl.

In certain embodiments, R¹³ is methyl.

In certain embodiments, R¹³ is haloalkyl.

In certain embodiments, R¹³ is CF₃.

### Embodiments of R¹⁴

In certain embodiments, R¹⁴ is hydrogen.

In certain embodiments, R¹⁴ is alkyl.

In certain embodiments, R¹⁴ is methyl.

In certain embodiments, R¹⁴ is ethyl.

In certain embodiments, R¹⁴ is haloalkyl.

In certain embodiments, R¹⁴ is CF₃.

In certain embodiments, R¹⁴ is aryl. In alternative embodiments, R¹⁴ is aryl optionally 15 substituted with 1, 2, or 3 substituents independently selected from R⁵.

In certain embodiments, R¹⁴ is heteroaryl. In alternative embodiments, R¹⁴ is heteroaryl optionally substituted with 1, 2, or 3 substituents independently selected from R⁵.

In certain embodiments, R¹⁴ is heterocycle. In alternative embodiments, R¹⁴ is heterocycle optionally substituted with 1, 2, or 3 substituents independently selected from R⁵.

In certain embodiments, R¹⁴ is —NH₂.

In certain embodiments, R¹⁴ is —N(H)(alkyl).

In certain embodiments, R¹⁴ is —N(alkyl)₂.

In certain embodiments, R¹⁴ is —OH.

In certain embodiments, R¹⁴ is alkoxy.

### Embodiments of R¹⁵

In certain embodiments, R¹⁵ is hydrogen.

In certain embodiments, each R¹⁵ is selected from alkyl, haloalkyl, and halogen.

In certain embodiments, R¹⁵ is alkyl.

In certain embodiments, R¹⁵ is haloalkyl.

In certain embodiments, R¹⁵ is alkenyl.

In certain embodiments, R¹⁵ is alkynyl.

In certain embodiments, R¹⁵ is halogen.

In certain embodiments, R¹⁵ is aryl.

In certain embodiments, R¹¹ is heteroaryl.

In certain embodiments, R¹¹ is heterocycle.

In certain embodiments, R¹¹ is cyano.

In certain embodiments, R¹¹ is nitro.

In certain embodiments, R¹⁵ is amino.

In certain embodiments, R¹⁵ is hydroxyl.

In certain embodiments, R¹⁵ is alkoxy.

In certain embodiments, R¹⁵ is —N(H)(alkyl).

In certain embodiments, R¹⁵ is —N(alkyl)₂.

### Embodiments of ¹²

In certain embodiments R¹² is

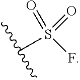 [SMILES: *S(=O)(=O)F]

In certain embodiments R¹² is

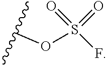 [SMILES: *OS(=O)(=O)F]

In certain embodiments R¹² is

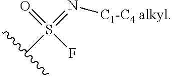

In certain embodiments R¹² is

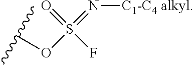

### Embodiments of R^{12B}

In certain embodiments R^{12B} is

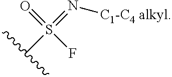

In certain embodiments R^{12B} is

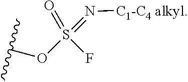

### Embodiments of R^{12C}

In certain embodiments R^{12C} is

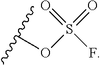 [SMILES: *OS(=O)(=O)F]

In certain embodiments R^{12C} is

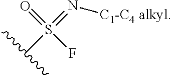

In certain embodiments R^{12C} is

### Embodiments of R¹⁶, R¹⁷, and R¹⁸

In certain embodiments, R¹⁶ is

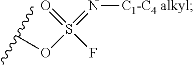

which is optionally substituted with 1 or 2 substituents independently selected from R⁵.

In certain embodiments, R¹⁶ is

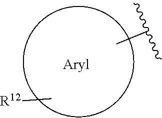

which is optionally substituted with 1 or 2 substituents independently selected from R⁵.

In certain embodiments, R¹⁶ is

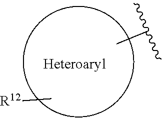

which is optionally substituted with 1 or 2 substituents independently selected from R⁵.

In certain embodiments, R¹⁶ is

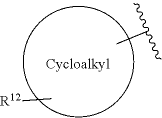

which is optionally substituted with 1 or 2 substituents independently selected from R⁵.

In certain embodiments, R¹⁶ is

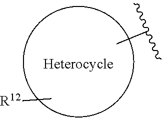

which is optionally substituted with 1 or 2 substituents independently selected from R⁵.

In certain embodiments, R¹⁶ is

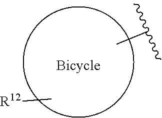

which is optionally substituted with 1 or 2 substituents independently selected from R⁵.

In certain embodiments, R¹⁶ is

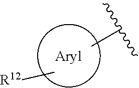

which is optionally substituted with 1 or 2 substituents independently selected from R⁵.

In certain embodiments, R^{l7} is

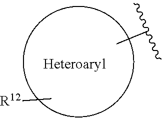

which is optionally substituted with 1 or 2 substituents independently selected from R⁵.

In certain embodiments, R¹⁷ is

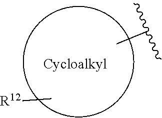

which is optionally substituted with 1 or 2 substituents independently selected from R⁵.

In certain embodiments, R¹⁷ is

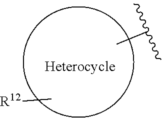

which is optionally substituted with 1 or 2 substituents independently selected from R⁵.

In certain embodiments, R¹⁷ is

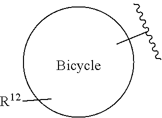

which is optionally substituted with 1 or 2 substituents independently selected from R⁵.

In certain embodiments, R¹⁸ is

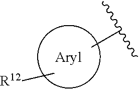

which is optionally substituted with 1 or 2 substituents independently selected from R⁵.

In certain embodiments, R¹⁸is

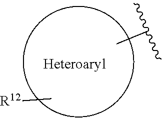

which is optionally substituted with 1 or 2 substituents independently selected from R⁵.

In certain embodiments, R¹⁸ is

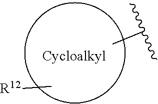

which is optionally substituted with 1 or 2 substituents independently selected from R⁵.

In certain embodiments, R¹⁸ is

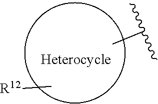

which is optionally substituted with 1 or 2 substituents independently selected from R⁵.

In certain embodiments, R¹⁸ is

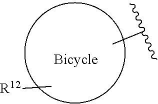

which is optionally substituted with 1 or 2 substituents independently selected from R⁵.

In certain embodiments, R¹⁶ and R¹⁷ are selected from the group consisting of

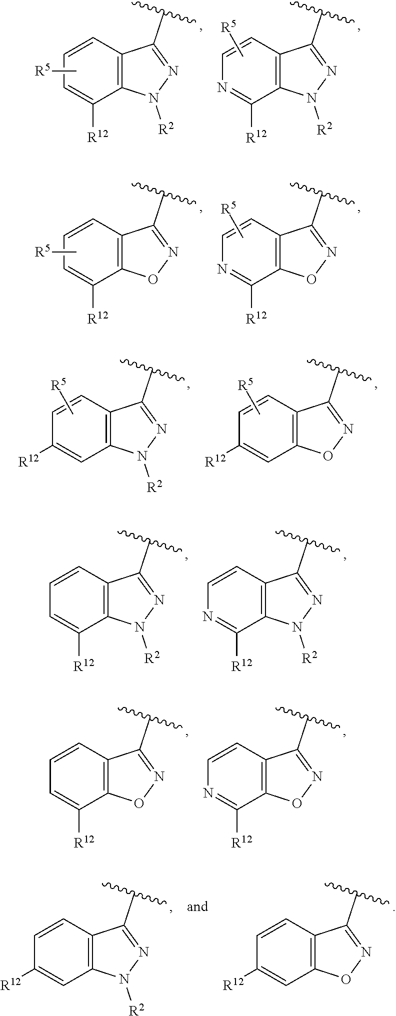

In certain embodiments, R¹⁶ and R¹⁷ are selected from the group consisting of

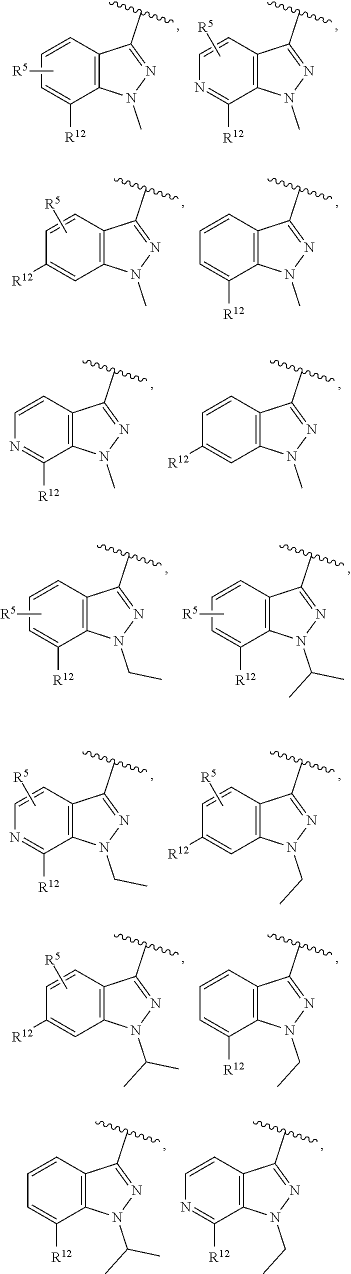

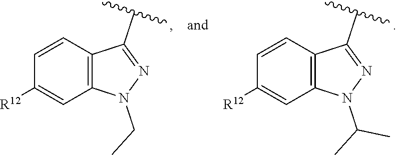

In certain embodiments, R¹⁶ and R¹⁷ are selected from

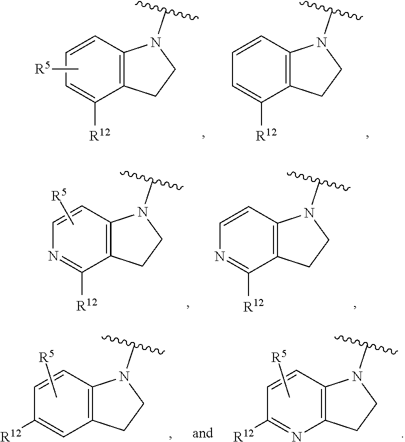

In certain embodiments, R⁸ is selected from

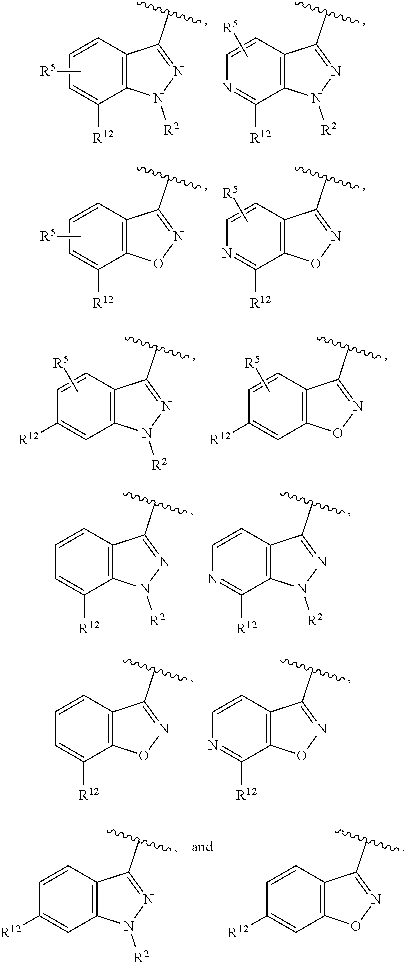

In certain embodiments, R⁸ is selected from

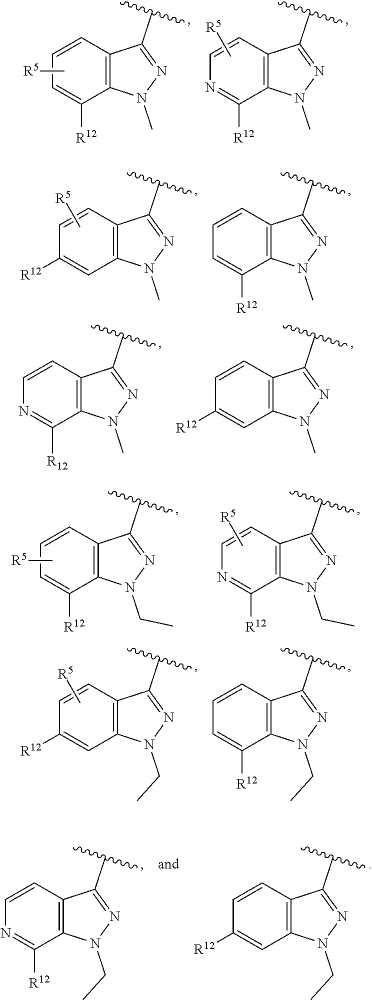

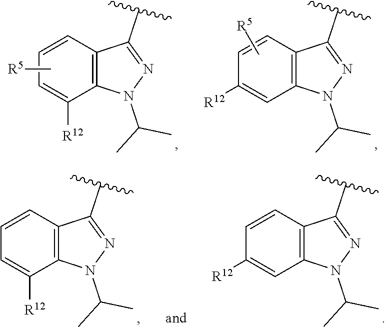

Embodiments of R1^{7A} and R1^{8A}

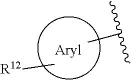

In certain embodiments, R¹7A is which is optionally substituted with 1 or 2 substituents independently selected from R⁵.

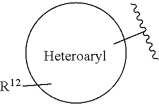

### Cycloalkyl

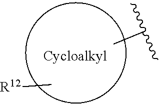

### Heterocycle

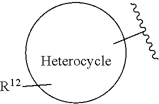

In certain embodiments, R¹7A is which is optionally substituted with 1 or 2 substituents independently selected from R⁵.

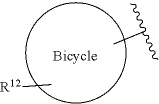

In certain embodiments, R¹⁷which is optionally substituted with 1 or 2 10 substituents independently selected from R⁵.

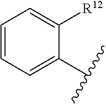

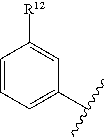

In certain embodiments, R^{17A}is which is optionally substituted with 1 or 2 substituents independently selected from R⁵.

### Aryl

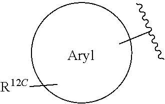

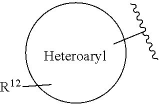

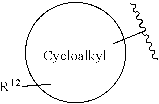

In certain embodiments, R¹8is which is optionally substituted with 1 or 2 substituents independently selected from R⁵.

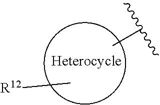

In certain embodiments, R¹ is which is optionally substituted with 1 or 2 substituents independently selected from R⁵.

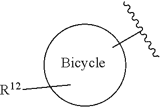

In certain embodiments, R¹8A is which is optionally substituted with 1 or 2 substituents independently selected from R⁵.

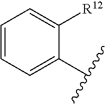

In certain embodiments, R¹8A is which is optionally substituted with 1 or 2 10 substituents independently selected from R⁵.

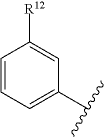

In certain embodiments, R¹8A is which is optionally substituted with 1 or 2 substituents independently selected from R⁵.

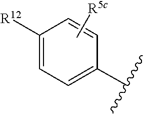

In certain embodiments, R¹8A is Which is optionally substituted with 1 or 2 substituents independently selected from R⁵.

In certain embodiments, R″ ^{7A} and R″ are selected from the group consisting of:

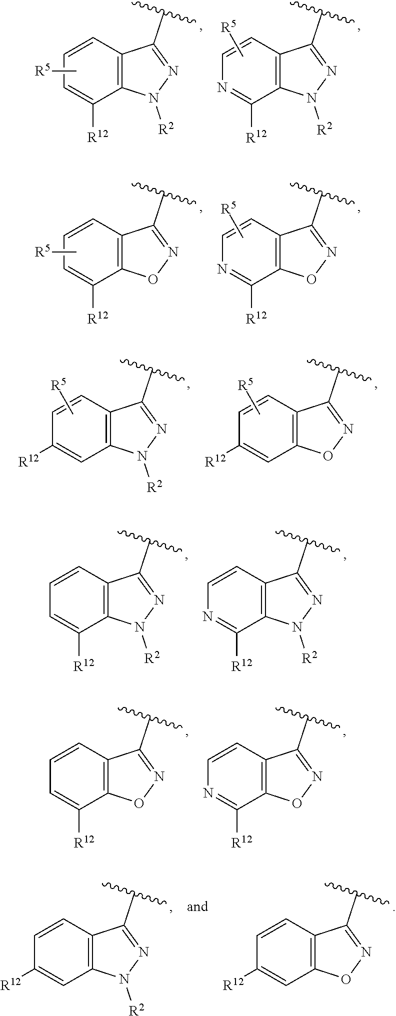

In certain embodiments, R^{17A} and R1⁸′ are selected from the group consisting of:

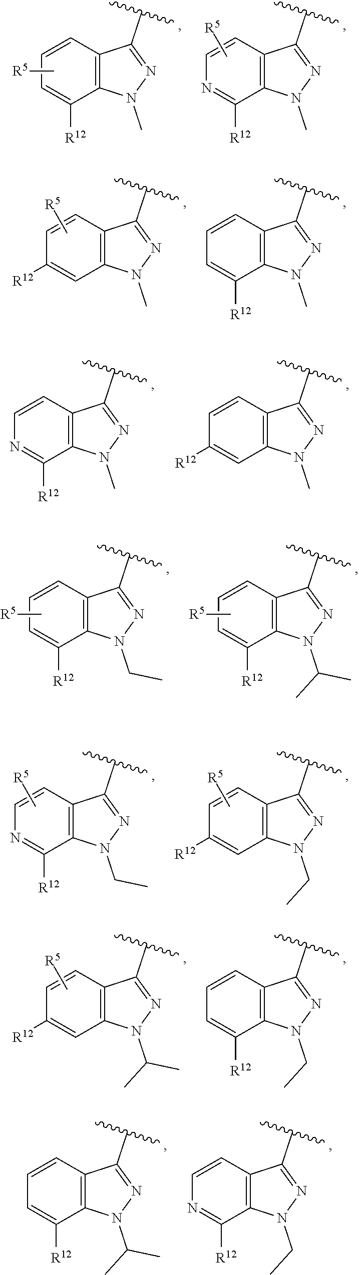

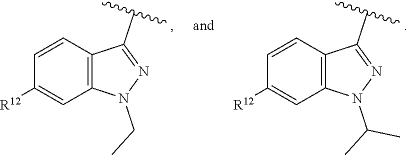

In certain embodiments, R^{17A} is selected from

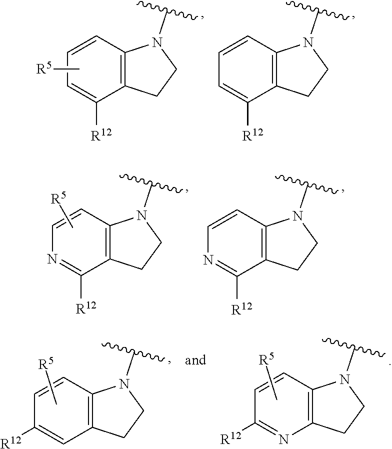

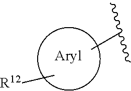

In certain embodiments, optionally substituted is selected from

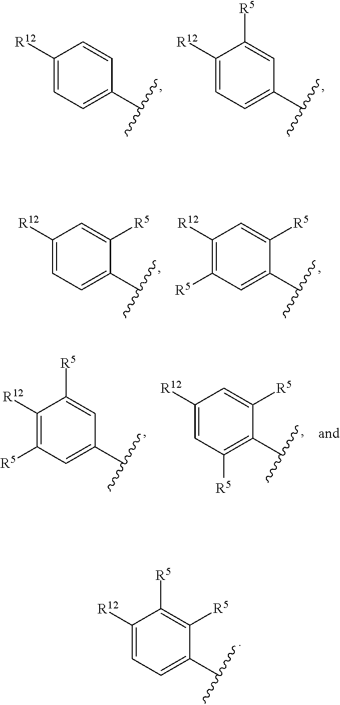

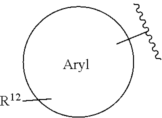

In certain embodiments, optionally substituted is selected from

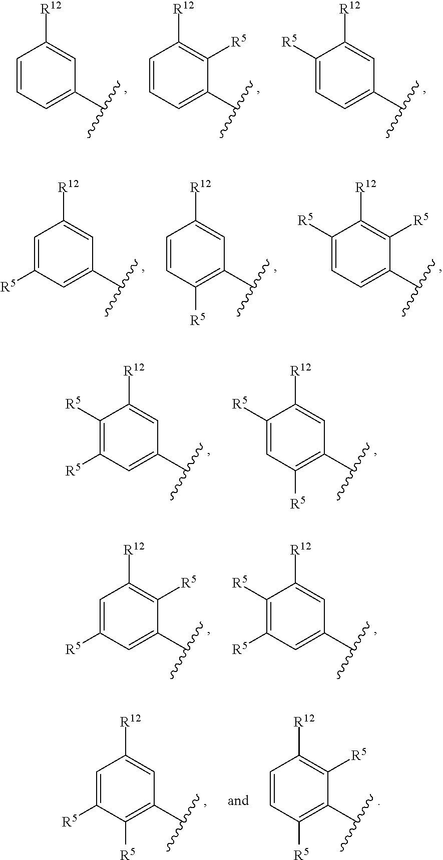

Aryl

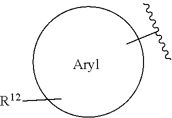

R12

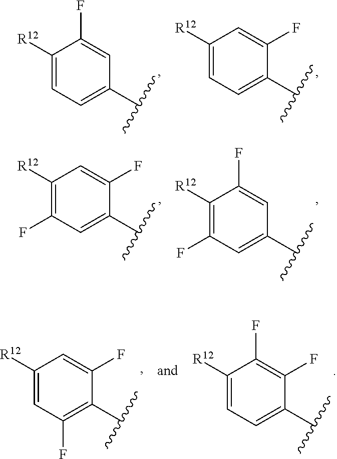

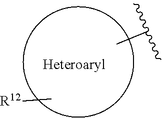

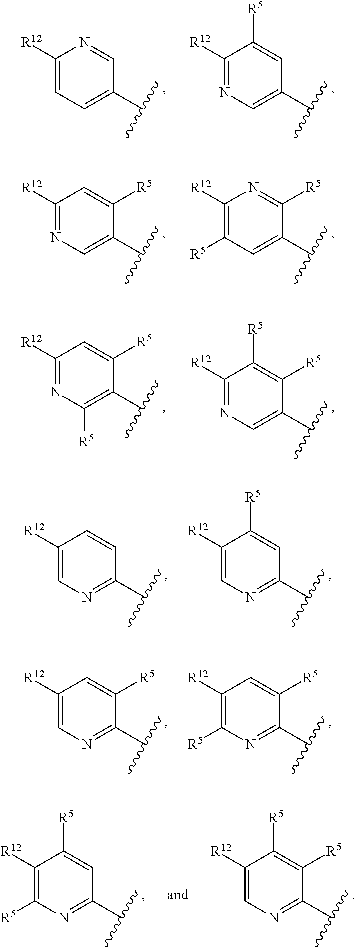

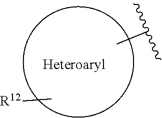

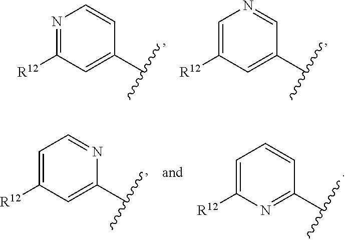

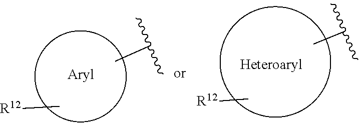

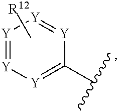

In certain embodiments, optionally substituted is selected from wherein each Y is independently selected from N, CH, or CRS, wherein 0, 1, or 2 (as context allows) instances of Y are selected 10 to be N and are selected to produce a stable ring as well known to those skilled in the art and that forms a pharmaceutically acceptable compound.

In certain embodiments, optionally substituted

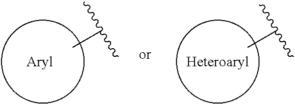

is

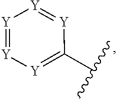

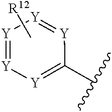

Non-limiting examples of include the following:

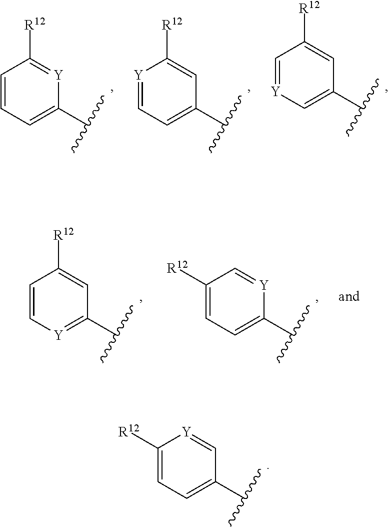

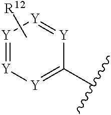

Additional examples of include the following:

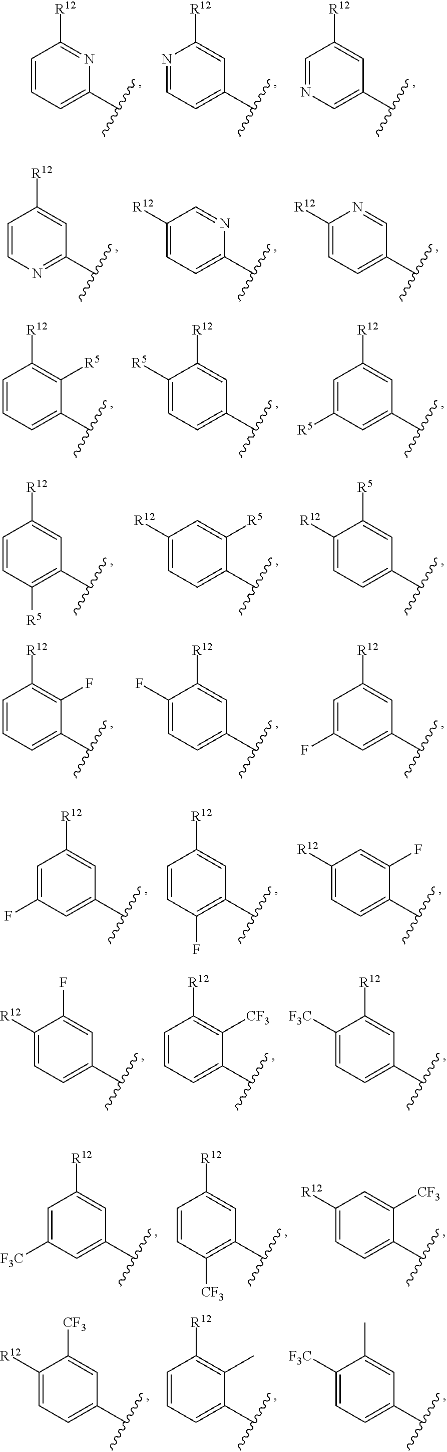

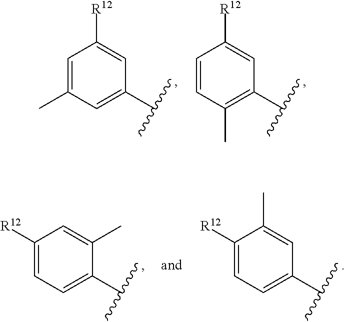

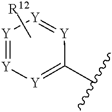

In certain embodiments,l is selected from:

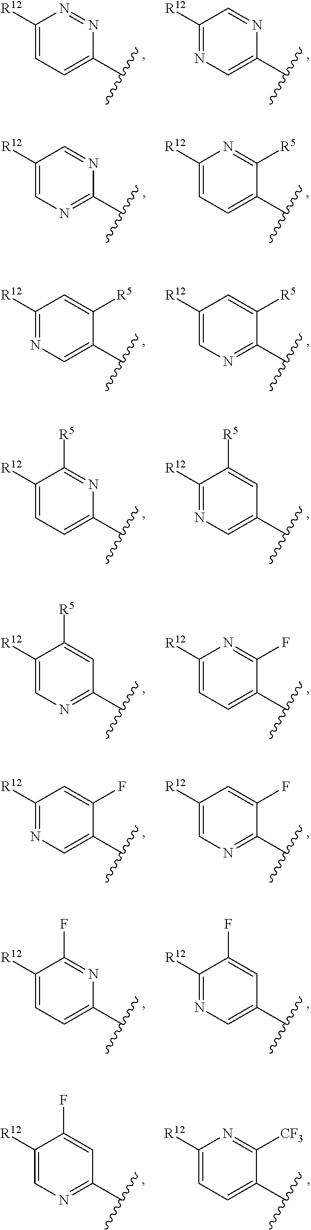

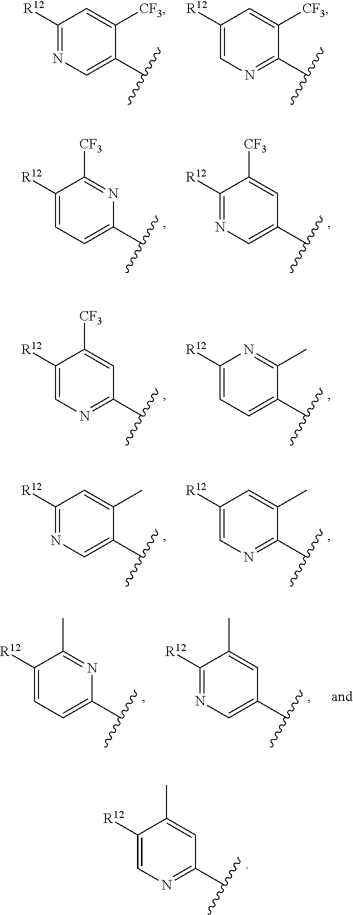

Non-limiting-examples of R¹⁶³ R¹⁷, and R″ ⁸ inclu

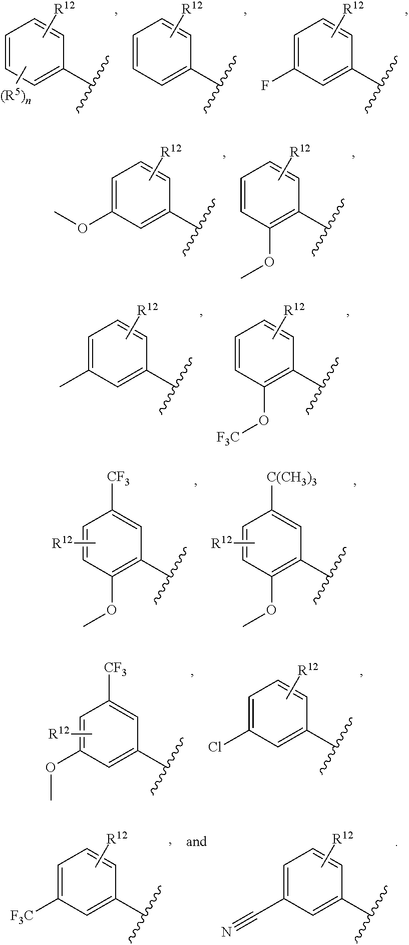

Non-limiting examples of R¹⁷a, and R1⁸′ include:

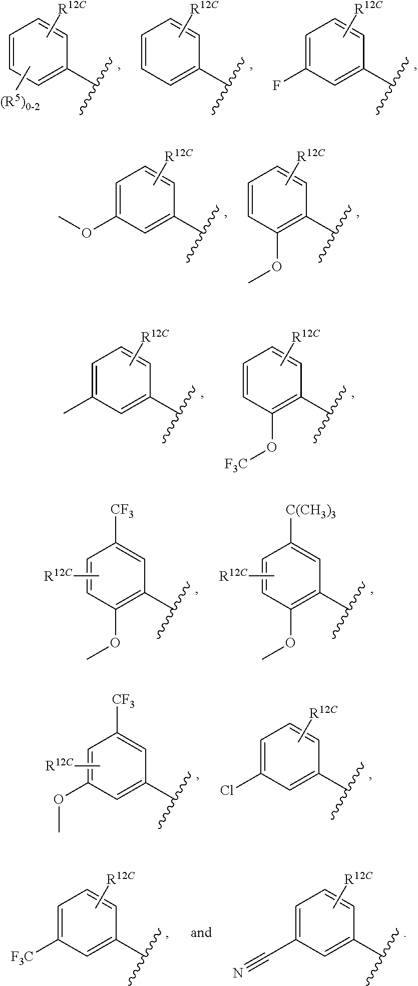

Embodiments of R^{16B}, R¹⁷B, and R^{18B}

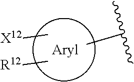

In certain embodiments, R¹⁶B is which is optionally substituted with 1 or 2 substituents independently selected from R⁵. x12 Heteroaryl R12

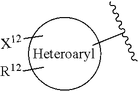

In certain embodiments, R16^{B} is which is optionally substituted 10 with 1 or 2 substituents independently selected from R⁵.

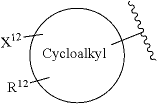

In certain embodiments, R¹⁶B is R¹² which is optionally substituted with 1 or 2 substituents independently selected from R⁵.

X12

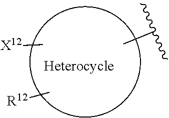

Heterocycle

In certain embodiments, R^{16B} is R¹² which is optionally substituted with 1 or 2 substituents independently selected from R⁵.

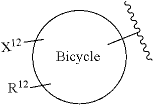

In certain embodiments, R^{16B} is which is optionally substituted with 1 or 2 substituents independently selected from R⁵.

X12 Aryl

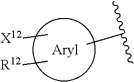

In certain embodiments, R^{17B} is which is optionally substituted with 1 or 2 substituents independently selected from R⁵.

X12 Heteroaryl

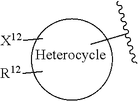

R12 In certain embodiments, R¹⁷B is which is optionally substituted with 1 or 2 substituents independently selected from R⁵.

X12 Cycloalkyl

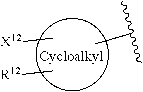

In certain embodiments, R^{17B} is R¹² which is optionally substituted with 1 or 2 substituents independently selected from R⁵. x12 Heterocycle

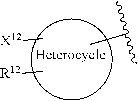

In certain embodiments, R^{17B} is R¹² which is optionally substituted 10 with 1 or 2 substituents independently selected from R⁵. x12 Bicycle

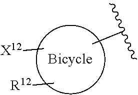

112 In certain embodiments, R^{17B}is which is optionally substituted with 1 or 2 substituents independently selected from R⁵.

X12 Aryl

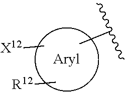

R12 In certain embodiments, R¹8B is which is optionally substituted with 1 or 2 substituents independently selected from R⁵.

X12 i

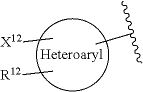

Heteroaryl R1 In certain embodiments, R^{18B} is which is optionally substituted with 1 or 2 substituents independently selected from R⁵.

X12

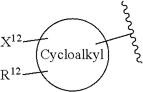

### Cycloalkyl R1

In certain embodiments, R^{18B} is which is optionally substituted with 1 or 2 substituents independently selected from R⁵.

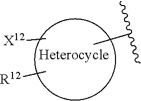

X1 Heterocycle

In certain embodiments, R^{18B} is R¹² which is optionally substituted with 1 or 2 substituents independently selected from R⁵.

### Bicycle

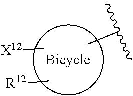

12 In certain embodiments, R¹⁸B is which is optionally substituted with 1 or 2 substituents independently selected from R⁵.

In certain embodiments, R^{16B}, R^{17B}, and R^{18B} are selected from the group consisting of:

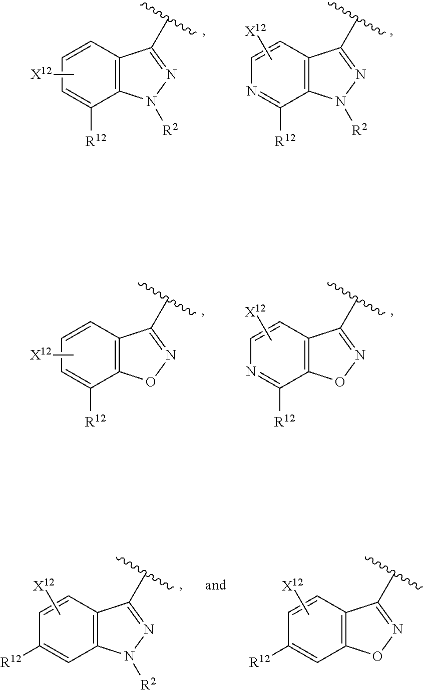

In certain embodiments R^{16B}, R^{17B} and R^{18B} are selected from the-group consisting of:

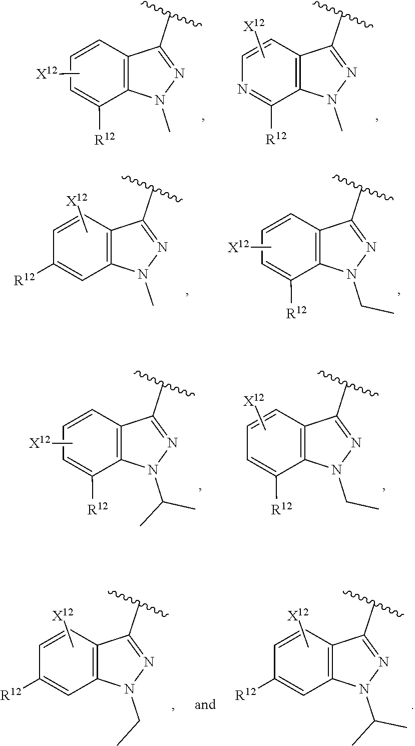

In certain embodiments, Re and R are selected from

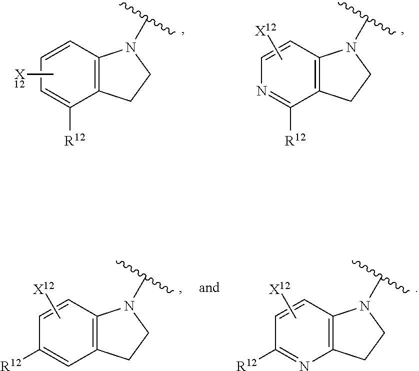

Embodiments of Degron In certain embodiments the Degron is selected from:

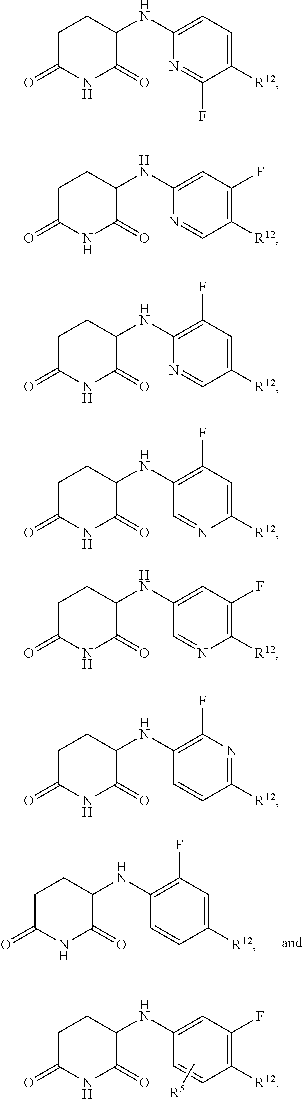

In certain embodiments the Degron is selected from:

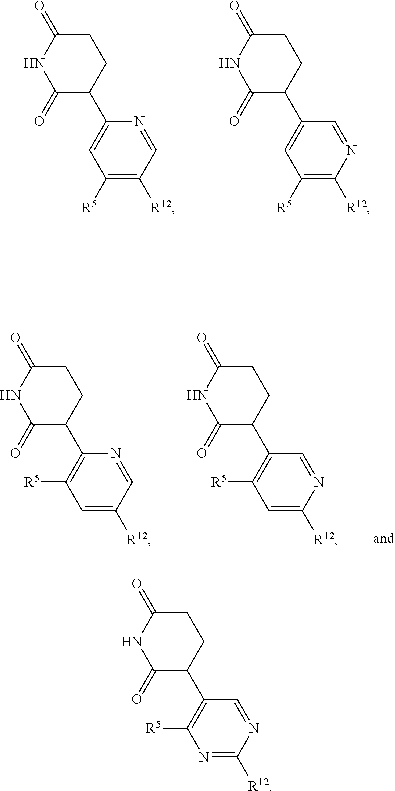

In certain embodiments the Degron is selected from:

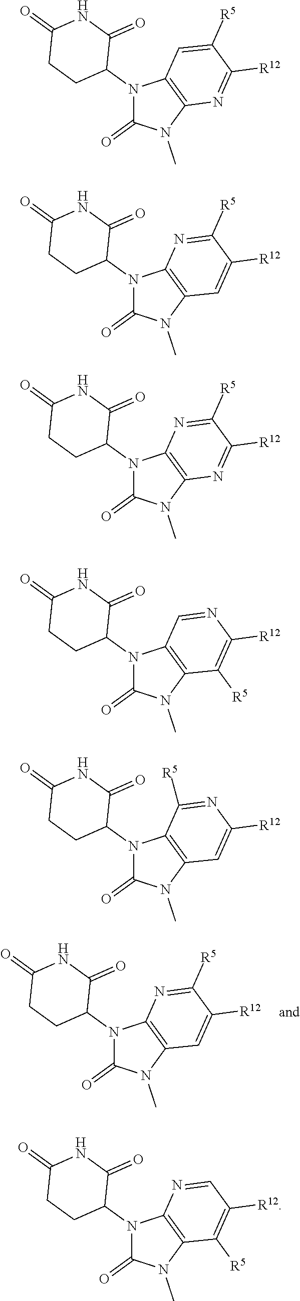

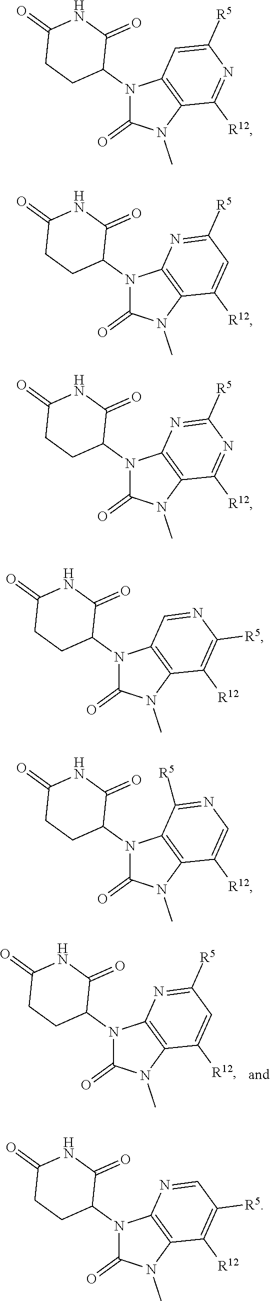

In certain embodiments the Degron is selected from:

In certain embodiments the Degron is selected from:

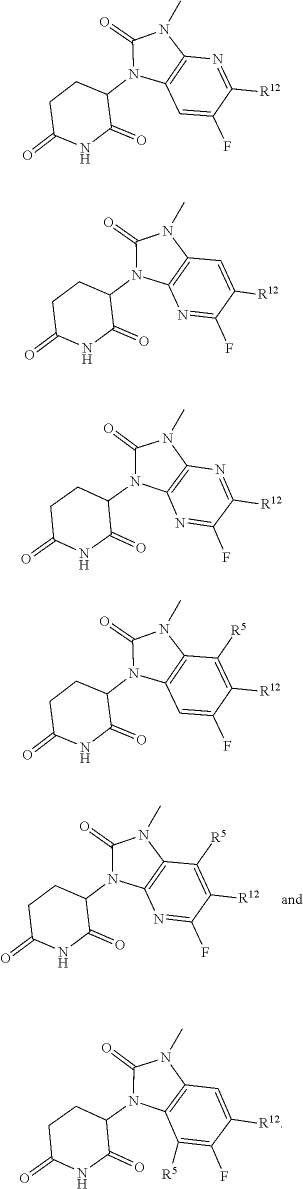

In certain embodiments the Degron is selected from:

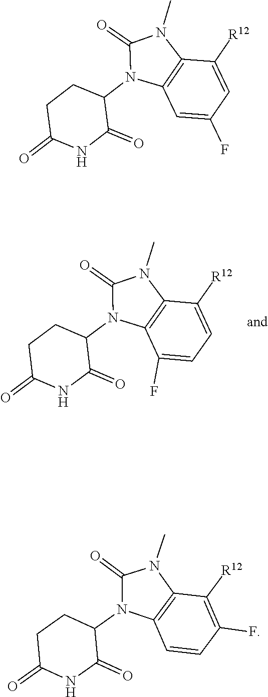

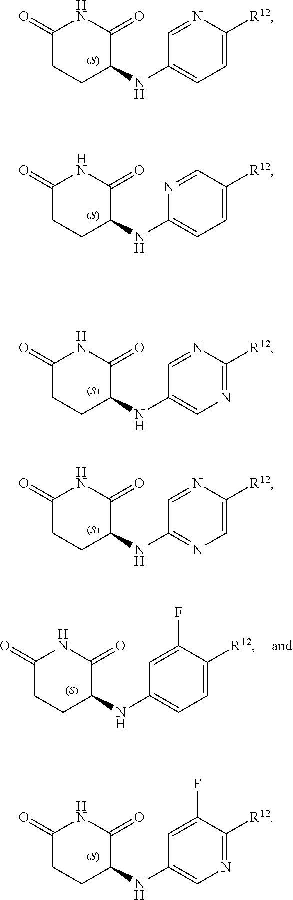

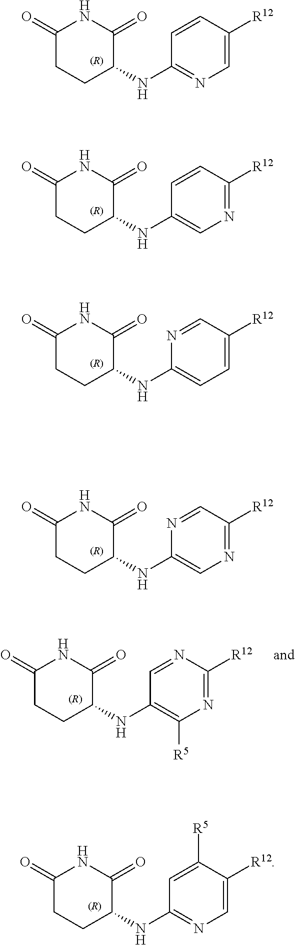

In certain embodiments the Degron is selected from:

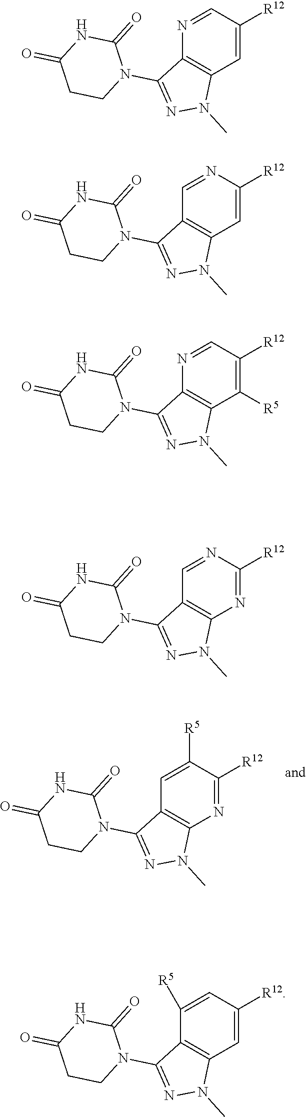

In certain embodiments the Degron is selected from:

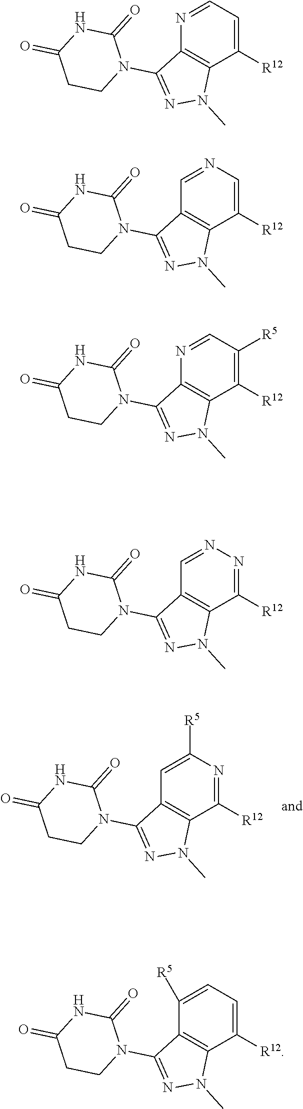

In certain embodiments the Degron is selected from:

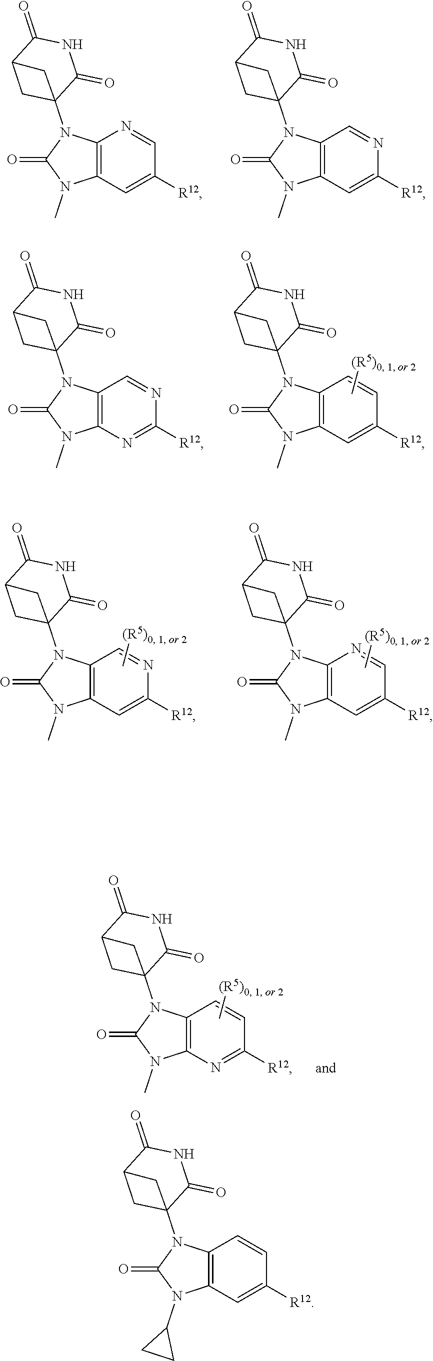

In certain embodiments the Degron is selected from:

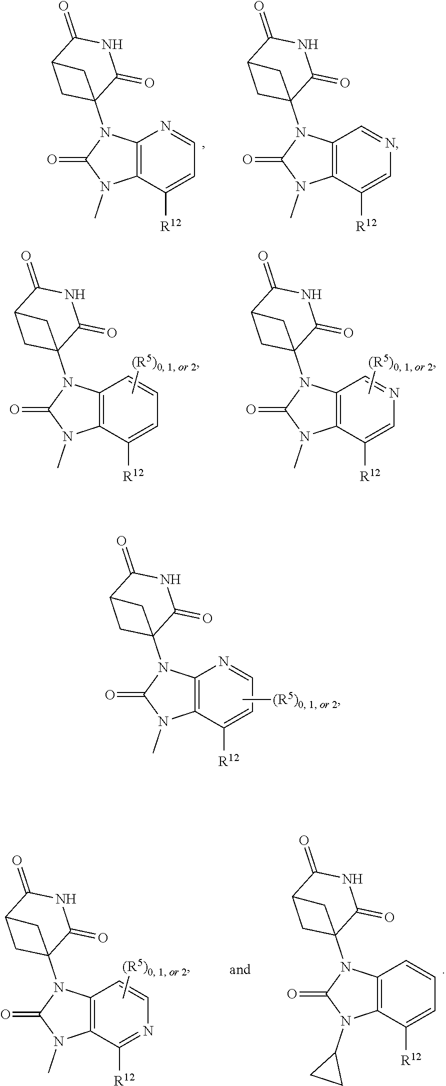

In certain embodiments the Degron is selected from:

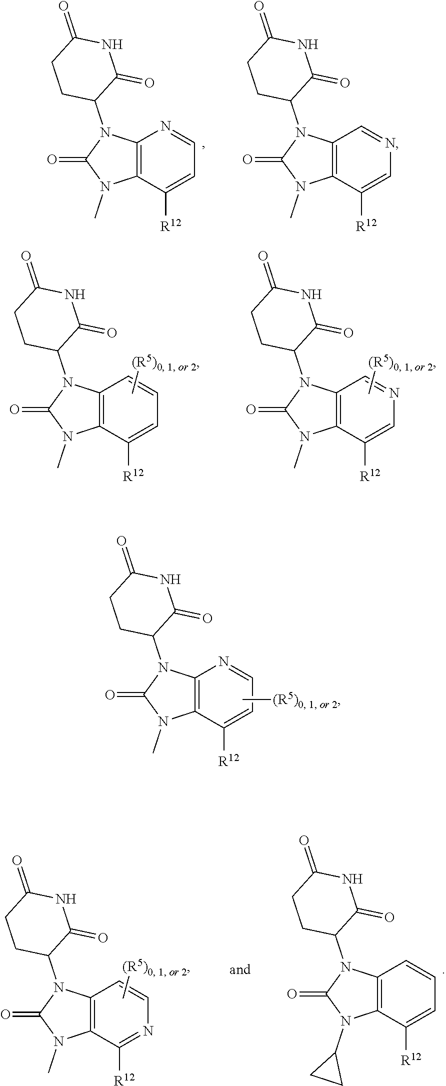

In certain embodiments the Degron is selected from:

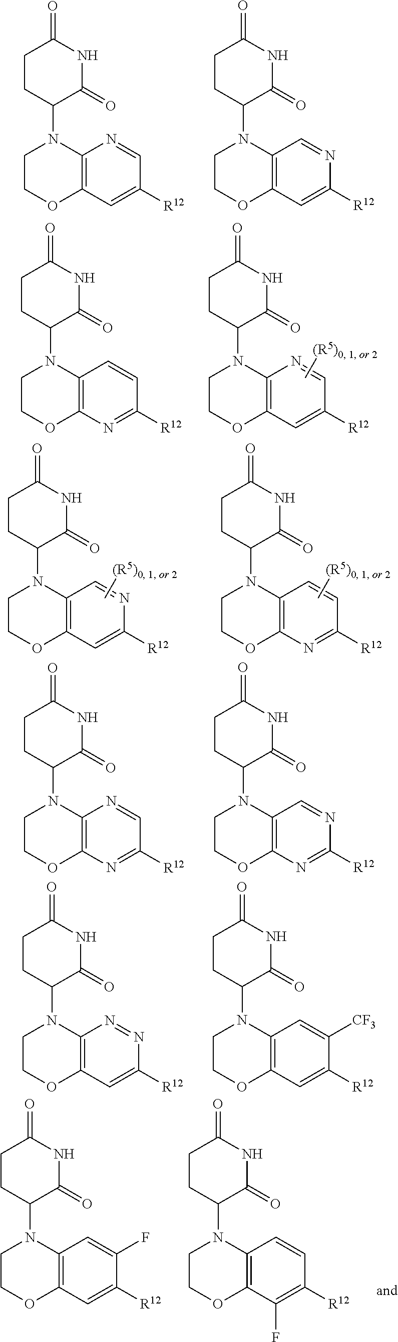

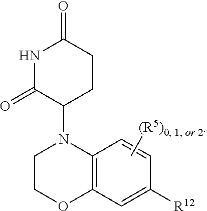

In certain embodiments the Degron is selected from:

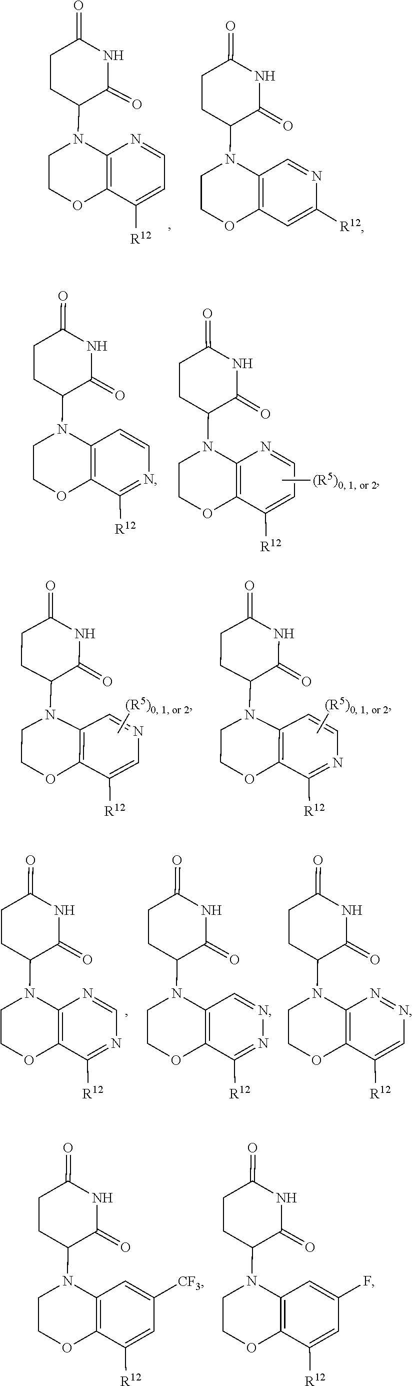

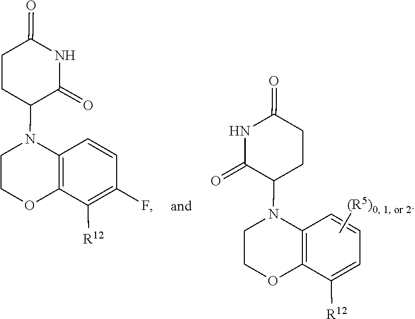

In certain embodiments the Degron is selected from:

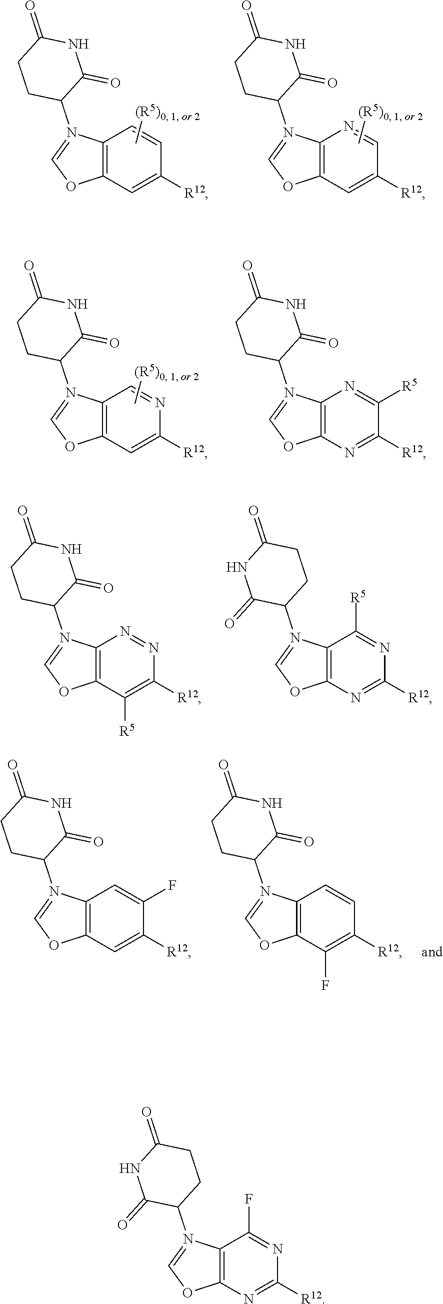

In certain embodiments the Degron is selected from:

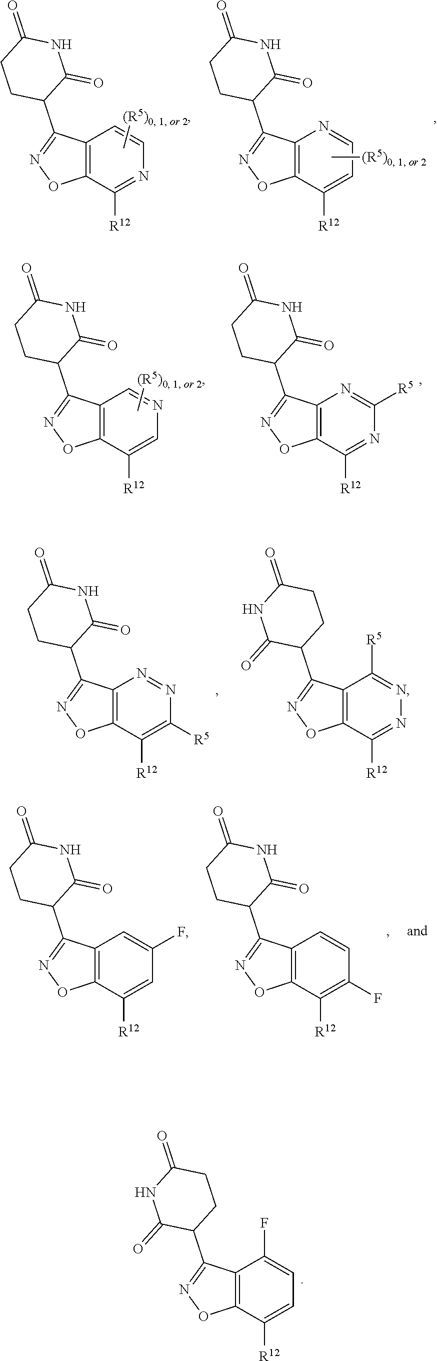

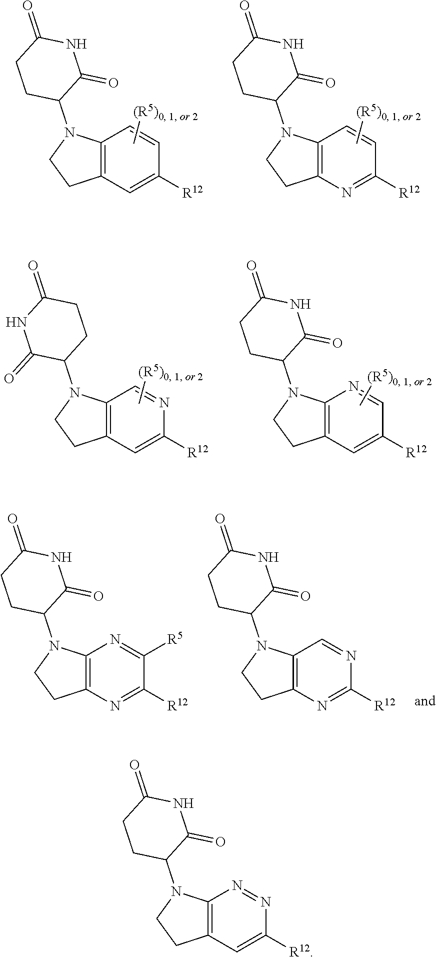

In certain embodiments the Degron is selected from:

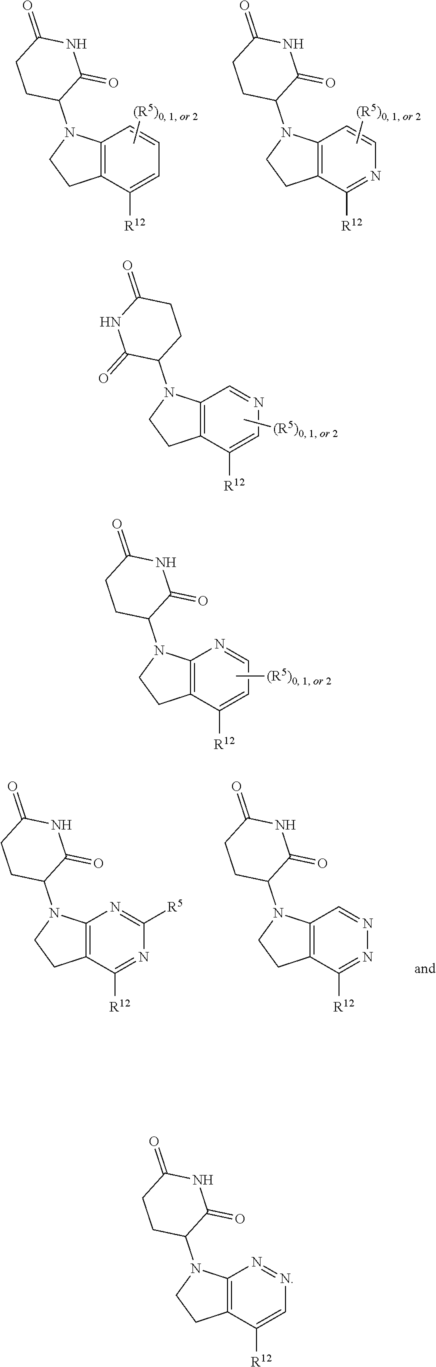

In certain embodiments the Degron is selected from:

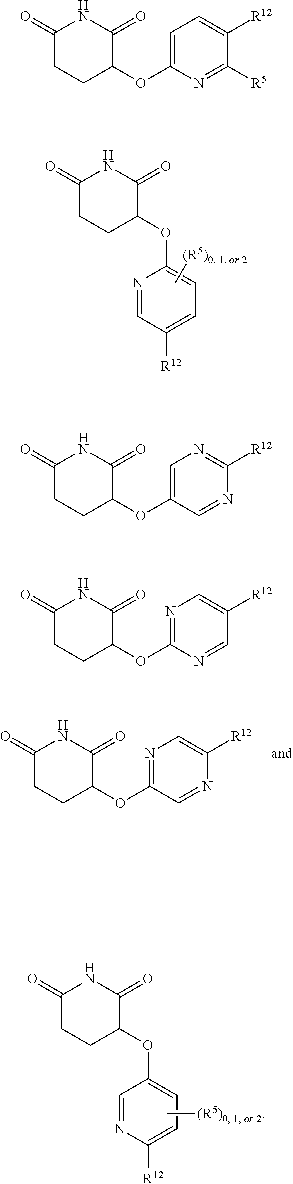

### 5 Embodiments of Tail

In certain embodiments, Tail is selected from the group consisting of:

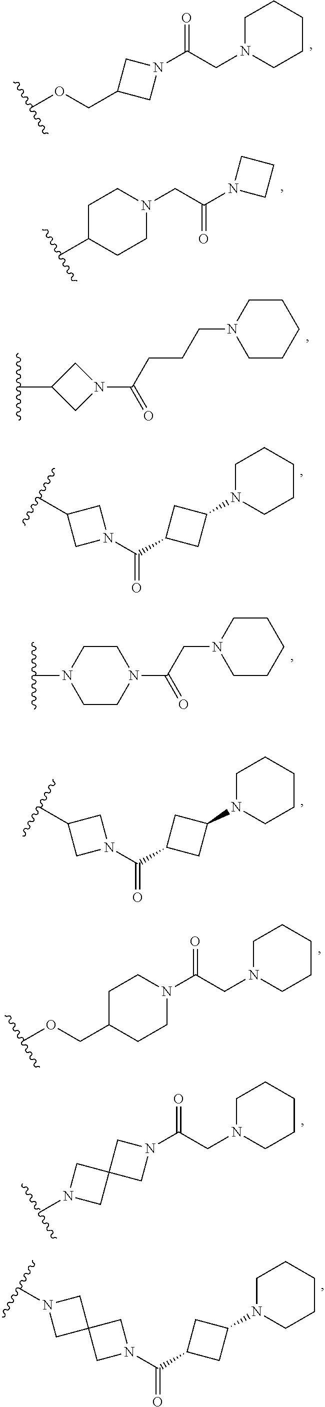

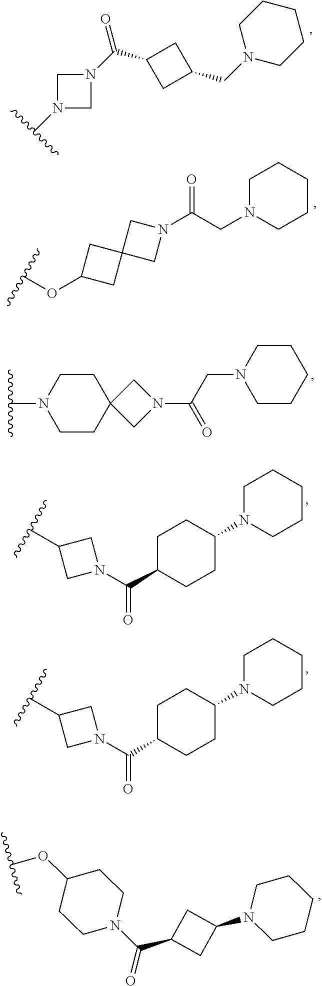 [SMILES: *N1CN(C(=O)[C@H]2C[C@@H](CN3CCCCC3)C2)C1 ; *OC1CC2(C1)CN(C(=O)CN1CCCCC1)C2 ; *N1CCC2(CC1)CN(C(=O)CN1CCCCC1)C2 ; *C1CN(C(=O)[C@H]2CC[C@H](N3CCCCC3)CC2)C1 ; *C1CN(C(=O)[C@H]2CC[C@@H](N3CCCCC3)CC2)C1 ; *OC1CCN(C(=O)[C@H]2C[C@@H](N3CCCCC3)C2)CC1]

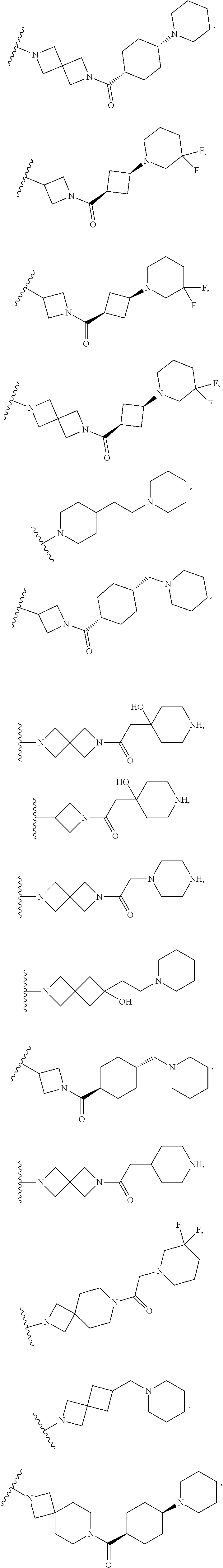

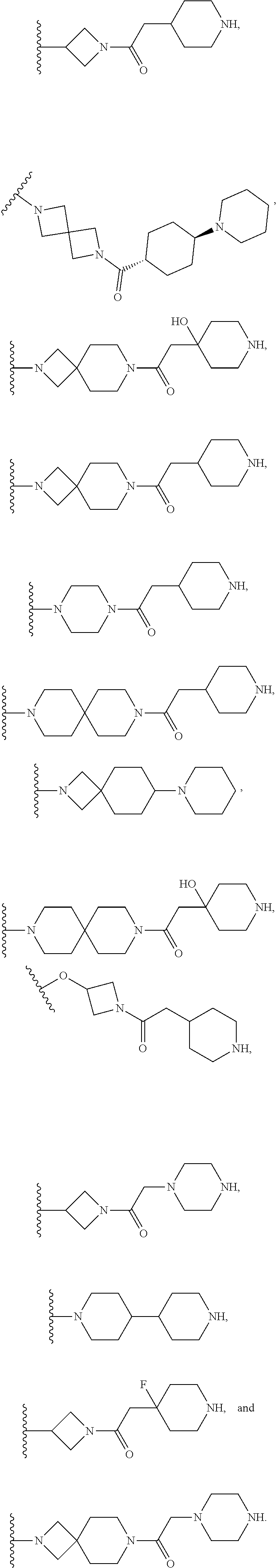

In certain embodiments, Tail is selected from the group consisting of:

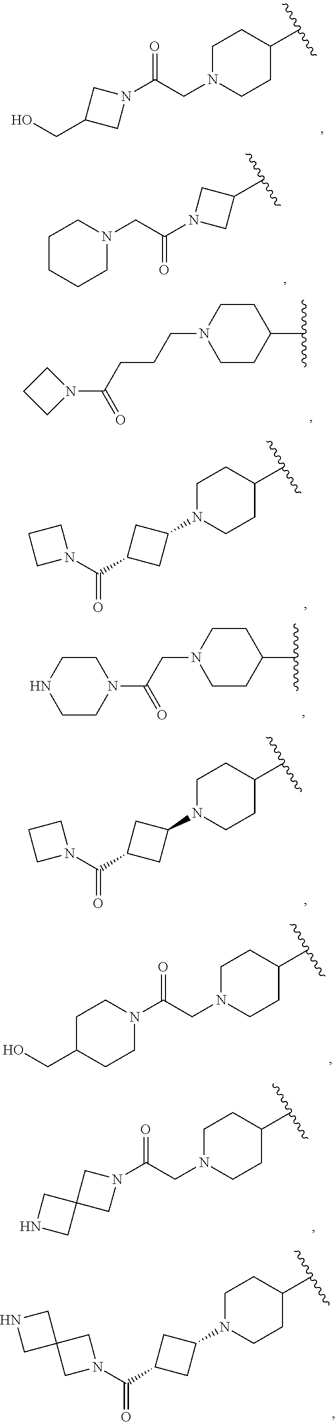

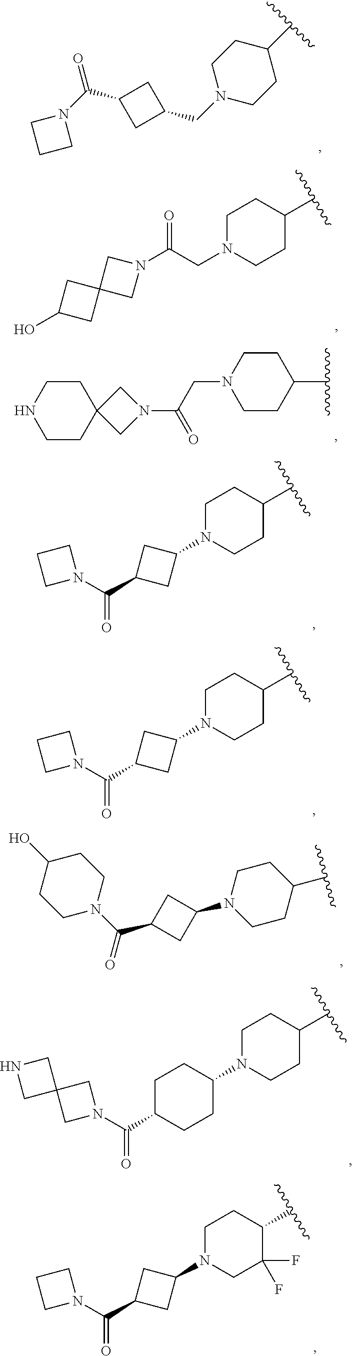

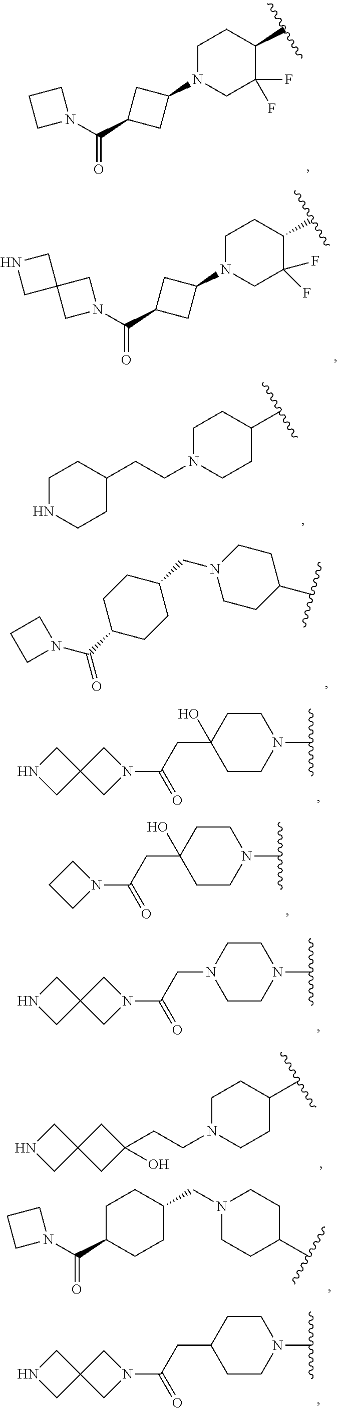

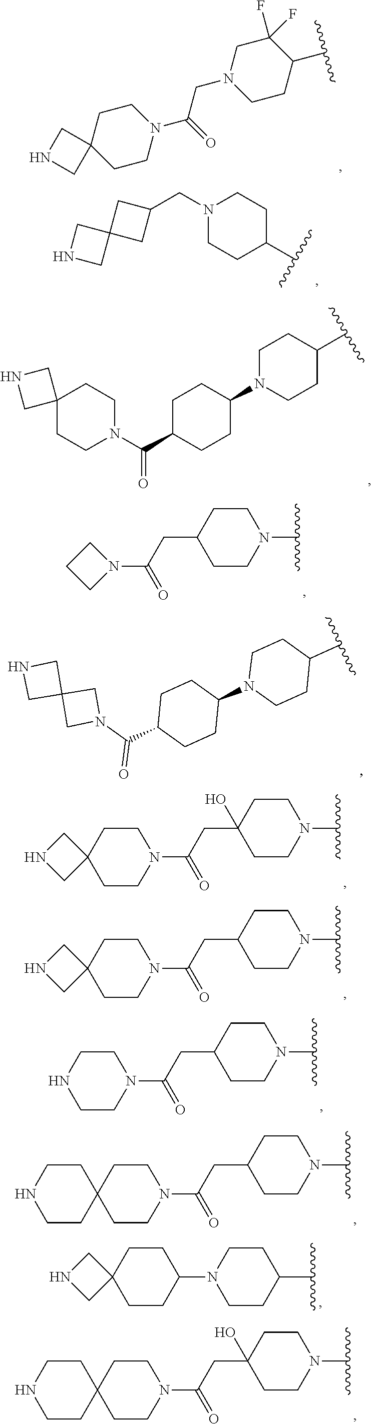

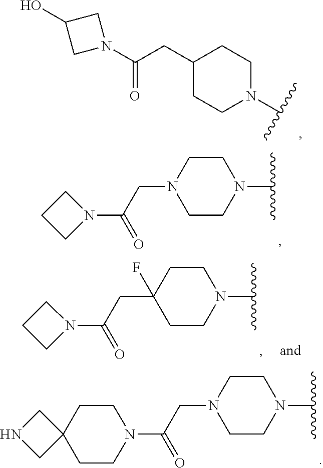

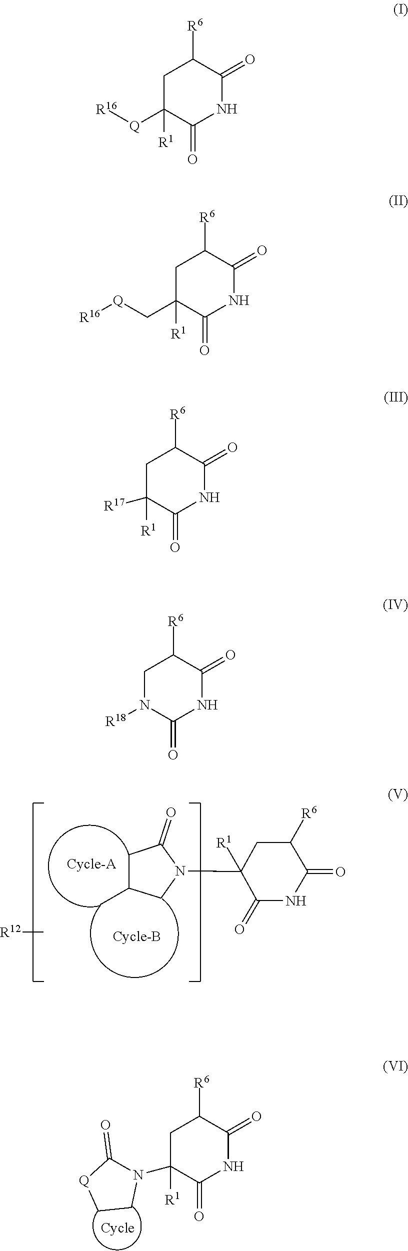

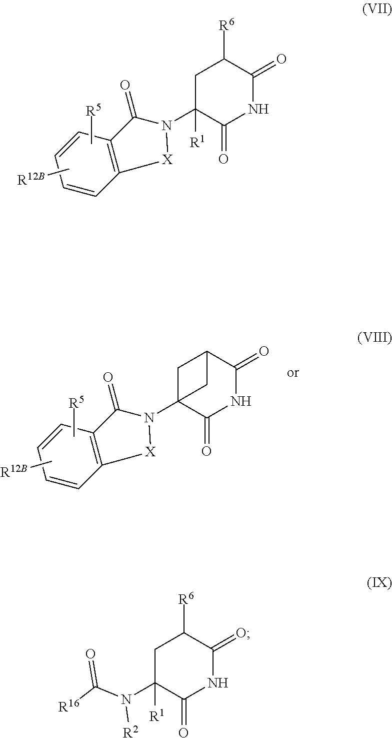

or a pharmaceutically acceptable salt thereof, wherein: Q is CH₂, NR2, O, or S; X is CH₂ or C(O); R¹ and R⁶ are independently selected from hydrogen, alkyl, alkenyl, alkynyl, and halogen;
or R¹ and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R⁵¹ is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a}; R¹⁶ is selected from:

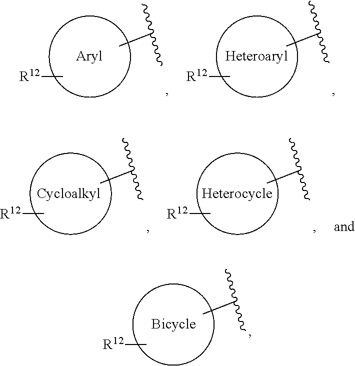

and, each of which is optionally substituted with 1 or 2 substituents independently selected from R⁵; R¹⁷ is selected from:

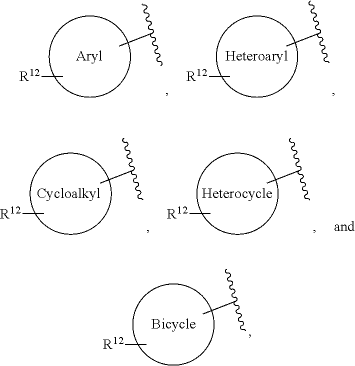

and, each of which is optionally substituted with 1 or 2 substituents 15 independently selected from R⁵;

R⁸ is selected from:

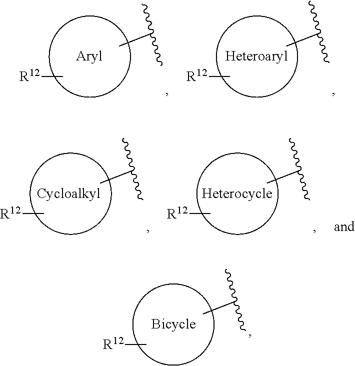

R12 and R each of which is attached to the azaglutarimide moiety through a C—N bond and each of which R¹¹ is optionally substituted with

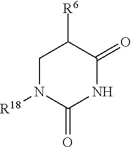

1 or 2 substituents independently selected from R⁵; for example o includes

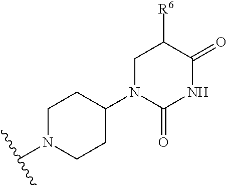

but does not include

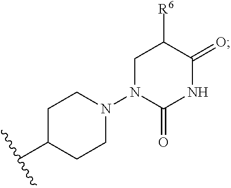

Cycle is a fused aryl or heteroaryl group optionally substituted with 1 or 2 substituents 5 independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a}.

Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1 or 2 substituents independently selected from R^{5b}_

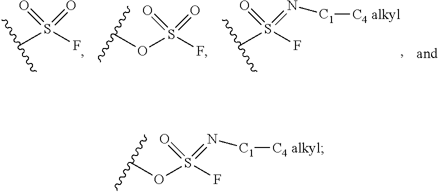

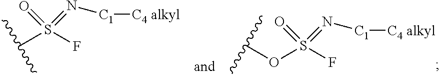

R⁷ and R at each instance are independently selected from hydrogen, alkyl, haloalkyl, alkenyl, alkynyl, aryl, heteroaryl, heterocycle, and C(O)R¹⁴ each of which except hydrogen is optionally substituted with 1 or 2 substituents independently selected from R1^{b}each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R^{10a}, and R^{10b} is independently selected from hydrogen, alkyl, haloalkyl, alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R″;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; and each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂.
or a pharmaceutically acceptable salt thereof. ^{PGP}-4²³,C¹

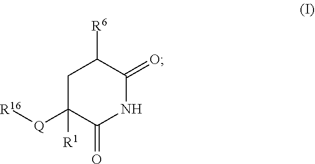

3. The compound of embodiment 1 wherein the compound is of Formula:

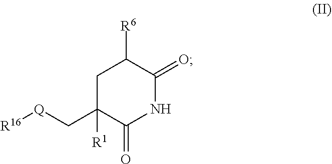

or a pharmaceutically acceptable salt thereof. 4. The compound of any one of embodiments 1-3, wherein Q is NH. 5. The compound of any one of embodiments 1-3, wherein Q is NCH₃. 6. The compound of any one of embodiments 1-3, wherein Q is CH₂. 7. The compound of any one of embodiments 1-3, wherein Q is O. 8. The compound of any one of embodiments 1-3, wherein Q is S. 9. The compound of embodiment 1 wherein the compound is of Formula:

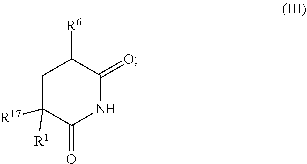

or a pharmaceutically acceptable salt thereof. 10. The compound of any one of embodiments 1-9, wherein R¹⁶ and R¹⁷ are

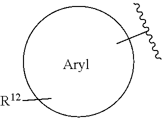

optionally substituted with 1, 2, or 3 substituents independently selected from R⁵. 11. The compound of any one of embodiments 1-9, wherein R¹⁶ and R¹⁷ are selected from the group consisting of:

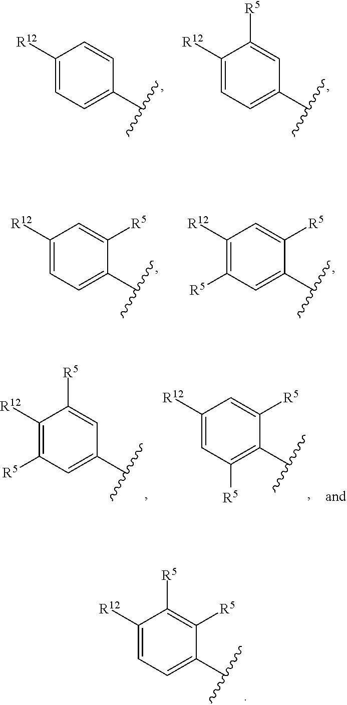

12. The compound of any one of embodiments 1-9, wherein R¹⁶ and R¹⁷ are selected from the group consisting of-

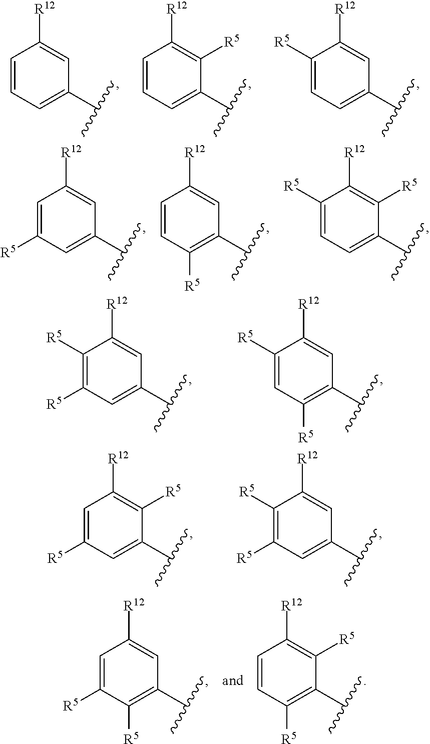

13. The compound of any one of embodiments 1-9, wherein R¹⁶ and R¹⁷ are selected from the group consisting of:

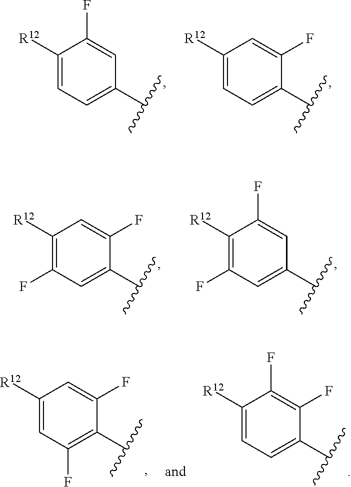

14. The compound of any one of embodiments 1-9, wherein R¹⁶ and R¹⁷ are

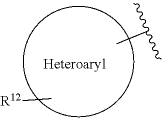

optionally substituted with 1, 2, or 3 substituents independently selected from R⁵. 15. The compound of any one of embodiments 1-9, wherein R¹⁶ and R¹⁷ are selected from the group consisting of:

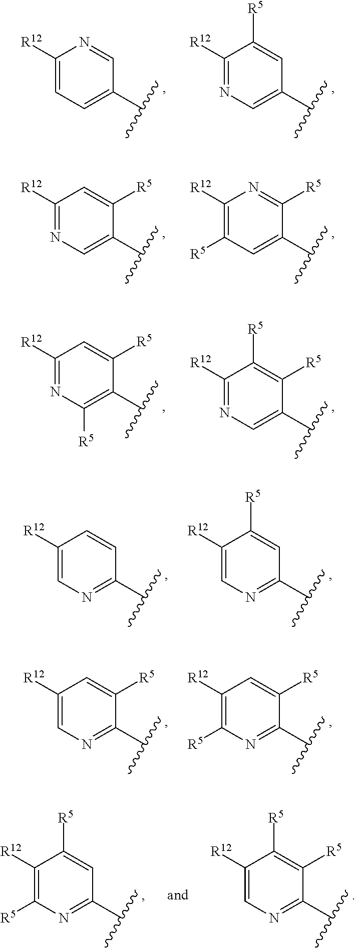

16. The compound of any one of embodiments 1-9, wherein R¹⁶ and R¹⁷ are selected from the group consisting of:

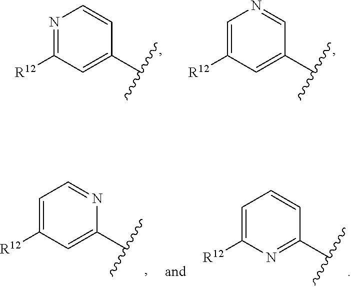

17. The compound of any one of embodiments 1-9, wherein R¹⁶ and R¹⁷ are selected from

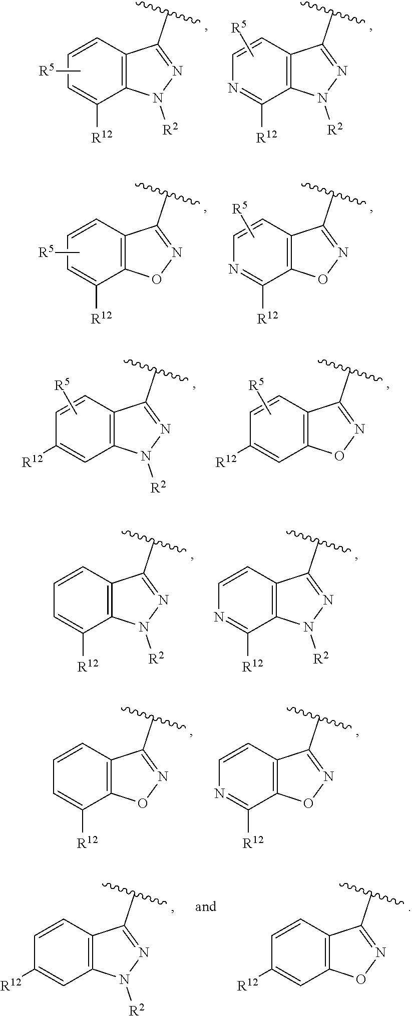

### and R12

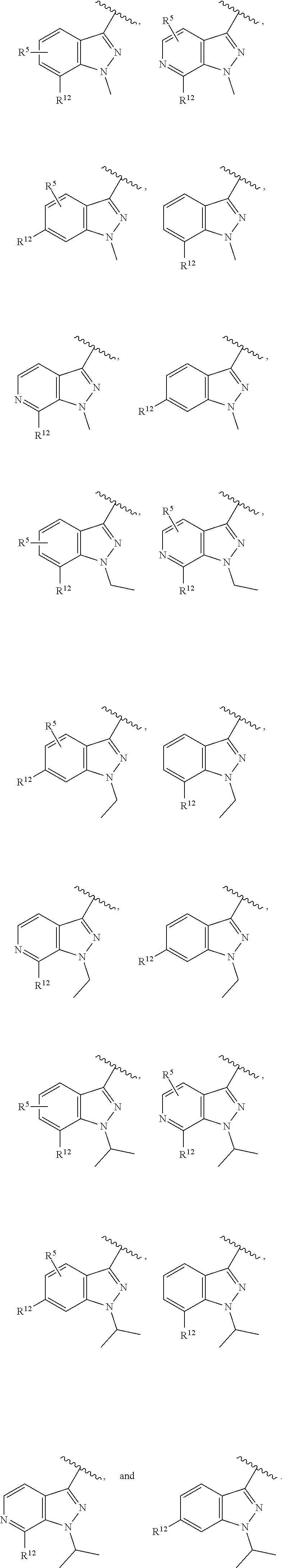

19. The compound of any one of embodiments 1-9, wherein R¹⁶ and R⁷ are selected from the group consisting of:

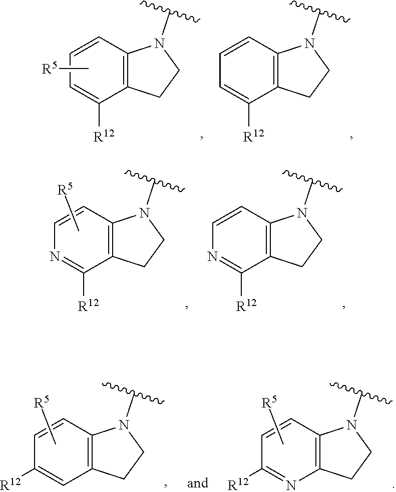

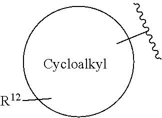

optionally substituted with 1, 2, or 3 substituents independently selected from R⁵. 21. The compound of any one of embodiments 1-9, wherein R¹⁶ and R¹⁷ are Heterocycle

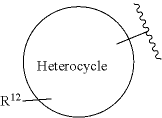

R12 optionally substituted with 1, 2, or 3 substituents independently selected from R⁵.

22. The compound of any one of embodiments 1-9, wherein R¹⁶ and R¹⁷ are

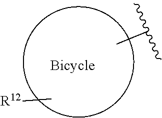

optionally substituted with 1, 2, or 3 substituents independently selected from R⁵. 23. The compound of embodiment 1 wherein the compound is of Formula:

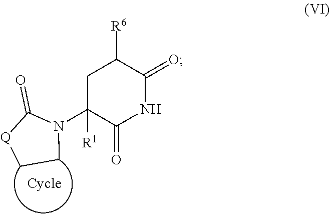

or a pharmaceutically acceptable salt thereof^{PGP}-4³⁹,C¹ 24. The compound of embodiment 23, wherein Cycle is phenyl optionally substituted with 1, 2, or 3 substituents independently selected from R⁵ and substituted with one R12 substituent. 25. The compound of any one of embodiments 23-24, wherein Q is NH. 26. The compound of any one of embodiments 23-24, wherein Q is NCH₃. 27. The compound of any one of embodiments 23-24, wherein Q is CH₂. 28. The compound of any one of embodiments 23-24, wherein Q is O. 29. The compound of any one of embodiments 23-24, wherein Q is S. 30. The compound of embodiment 1 wherein the compound is of Formula:

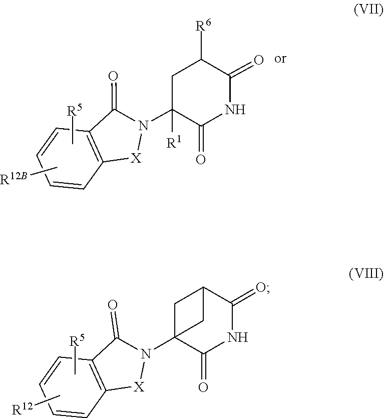

or a pharmaceutically acceptable salt thereof. 31. The compound of embodiment 30, wherein X is CH₂. 32. The compound of embodiment 30, wherein X is C(O). 33. The compound of any one of embodiments 1-32, wherein R¹ is hydrogen. 34. The compound of any one of embodiments 1-32, wherein R¹ is CH₃. 35. The compound of any one of embodiments 1-32, wherein R¹ and R⁶ combined form a one-carbon bridge. 36. The compound of any one of embodiments 1-34, wherein R⁶ is hydrogen. 37. The compound of any one of embodiments 1-34, wherein R⁶ is CH₃. 38. The compound of embodiment 1 wherein the compound is of Formula: R⁶ Ra N NH O (IV)

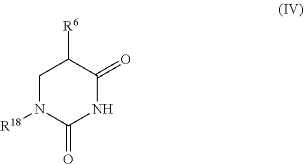

or a pharmaceutically acceptable salt thereof. ^{PGP}-4⁴¹,C¹ 39. The compound of embodiment 38, wherein R⁶ is hydrogen. 40. The compound of embodiment 38, wherein R⁶ is CH₃. 41. The compound of any one of embodiments 38-40, wherein R¹⁸ is Aryl

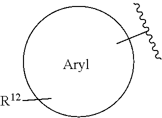

R12 optionally substituted with 1 or 2 substituents independently selected from R⁵. 42. The compound of any one of embodiments 38-40, wherein R¹⁸ is Heteroaryl R12

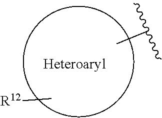

whi 1s3′attached to the azaglutarimide moiety through a C—N bond and optionally substituted with 1 or 2 substituents independently selected from R⁵.

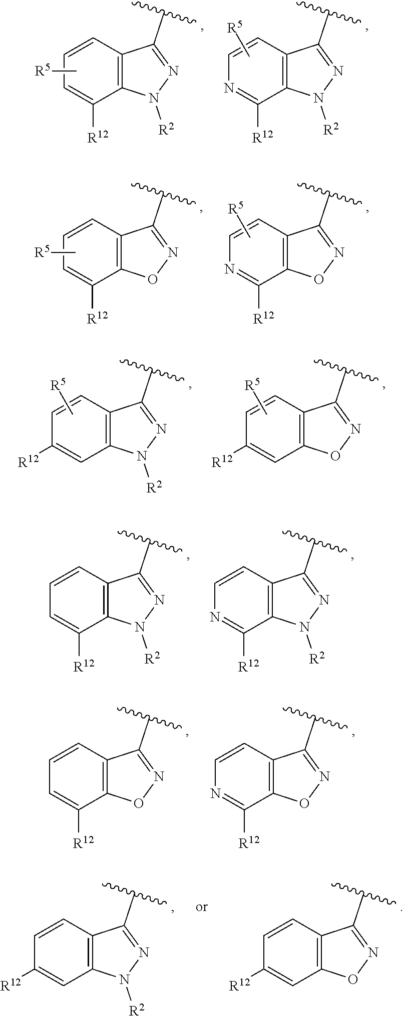

44. The compound of any one of embodiments 38-40, wherein R is

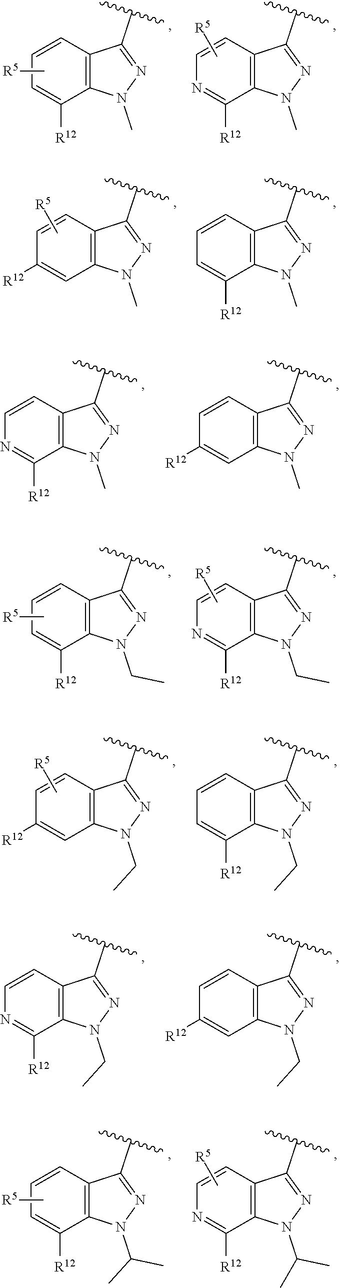

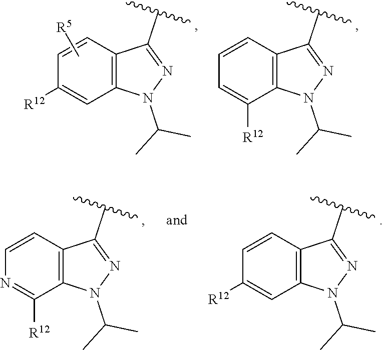

45. The compound of any one of embodiments 1-44, wherein each R⁵ is independently^{PGP}-4⁴⁵,C³ selected from hydrogen, alkyl, haloalkyl, and halogen. 46. The compound of any one of embodiments 1-44, wherein each R⁵ is hydrogen. 47. The compound of any one of embodiments 1-44, wherein one R⁵ is F. 48. The compound of any one of embodiments 1-44, wherein R⁵ is —NR⁷R or —OR⁷. 49. The compound of embodiment 48, wherein R⁷ is hydrogen. 50. The compound of embodiment 48, wherein R is methyl. 51. The compound of any one of embodiments 48-50, wherein R⁸ is hydrogen. 52. The compound of any one of embodiments 48-50, wherein R is methyl. 53. The compound of any one of embodiments 1-44, wherein R⁵ is aryl or heteroaryl optionally substituted with 1, 2, or 3 substituents independently selected from R¹⁰. 54. The compound of any one of embodiments 1-44, wherein R⁵ is cyano. 55. The compound of any one of embodiments 1-44, wherein R⁵ is nitro. 56. The compound of any one of embodiments 1-44, wherein R⁵ -C(O)CH₃. 57. The compound of embodiment 1 wherein the compound is of Formula:

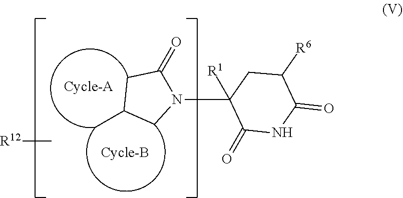

or a pharmaceutically acceptable salt thereof. 58. The compound of embodiment 57, wherein Cycle-A is phenyl optionally substituted with 1 or 2 substituents independently selected from Rs. 59. The compound of embodiment 57 or 58, wherein Cycle-B is phenyl optionally substituted with 1 or 2 substituents independently selected from R^{5b}. 60. The compound of any one of embodiments 57-59, wherein R¹ is hydrogen. 61. The compound of any one of embodiments 57-59, wherein R¹ is CH₃. 62. The compound of any one of embodiments 57-59, wherein R¹ and R⁶ combined form a one-carbon bridge. 63. The compound of any one of embodiments 57-61, wherein R⁶ is hydrogen. 64. The compound of any one of embodiments 57-61, wherein R⁶ is CH₃.

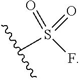 [SMILES: *S(=O)(=O)F]

66. The compound of any one of embodiments 1-64, wherein R¹² is F

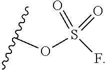 [SMILES: *OS(=O)(=O)F]

N. -C₁-C₄ alkyl 67. The compound of any one of embodiments 1-64, wherein R12 is F

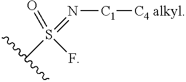

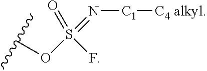

68. The compound of any one of embodiments 1-64, wherein R¹² is O F

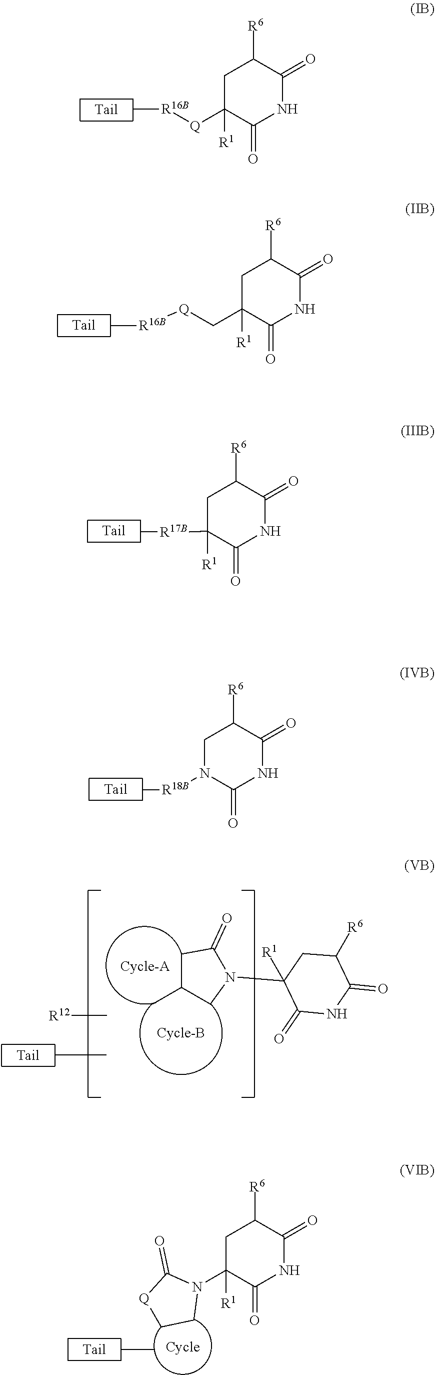

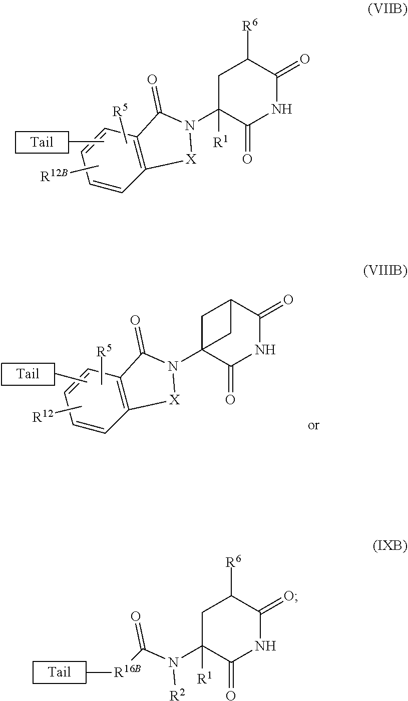

69. A compound of Formula:

or a pharmaceutically acceptable salt thereof, 5 wherein: Q is CH₂, NR2, O, or S; X is CH₂ or C(O); R¹ and R⁶ are independently selected from hydrogen, alkyl, alkenyl, alkynyl, and halogen;
or R¹ and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R″ °;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a};

Tail is selected from X³¹i R23 R21 X22.

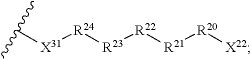

X³¹ is selected from bond, heterocycle, aryl, heteroaryl, bicycle, —NR²⁷—, —CR⁴“R⁴¹-, -0-, —C(O)—, —C(NR²⁷)—, —C(S)—, —S(O)—, —S(O)2- and -5-; each of which heterocycle, aryl, heteroaryl, and bicycle is substituted with 1, 2, or 3 substituents independently selected from R40,

X²² is selected from hydrogen, alkyl, haloalkyl, alkenyl, alkynyl, halogen, heteroaryl, heterocycle, —NR⁷R⁸, —OR⁷, —SR⁷, —C(O)R⁹, —C(S)R⁹, —S(O)R⁹, —S(O)₂R⁹, —OC(O)R⁹, —OC(S)R⁹, —OS(O)R⁹, —OS(O)₂R⁹, —SC(O)R⁹, —OS(O)₂R⁹, —NR⁷C(O)R⁹, —NR⁷C(S)R⁹, —NR⁷S(O)R⁹, —NR⁷S(O)₂R⁹, —P(O)(R⁹)₂, —SP(O)(R⁹)₂, —NR⁷P(O)(R⁹)₂, and —OP(O)(R⁹)₂; R²⁰, R²¹, R²², R²³, and R²⁴ are independently at each occurrence selected from the group consisting of a bond, alkyl, —C(O)—, —C(O)O—, —OC(O)—, —SO2-, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, heteroaryl, and carbocycle; each of which is optionally substituted with 1, 2, or 3 substituents independently selected from R⁴⁰; R²⁶ is independently at each occurrence selected from the group consisting of hydrogen, alkyl, arylalkyl, heteroarylalkyl, alkene, alkyne, aryl, heteroaryl, and heterocycle; R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkene, and alkyne; R⁴⁰ is independently at each occurrence selected from the group consisting of hydrogen, alkyl, alkene, alkyne, fluoro, bromo, chloro, azide, OR⁷, NR⁷R⁸, S(O)NR⁷R⁸, S(O)2NR⁷R⁸, S(O)OR⁷, S(O)₂OR⁷, haloalkyl, aryl, heteroaryl, heterocycle, and cycloalkyl; R⁴¹ is independently at each occurrence selected from the group consisting of hydrogen, alkyl, alkene, alkyne, haloalkyl, aryl, heteroaryl, heterocycle, and cycloalkyl; R¹⁶B is selected from:

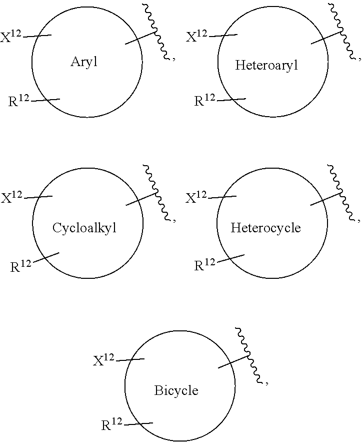

each of which is optionally substituted with 1 or 2 substituents independently selected from R⁵; R″ ” is selected from:

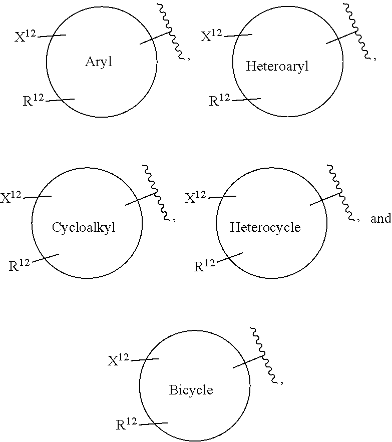

each of which is optionally substituted with 1 or 2 substituents independently selected from R⁵;

Ri⁸B is selected from:

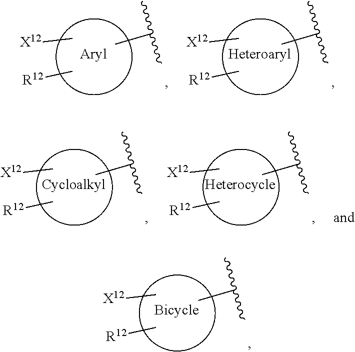

each of which is attached to the azaglutarimide moiety through a C—N bond and each of which Ri⁸B is optionally substituted with 1 or 2 substituents independently selected from R⁵; X¹² is the attachment point to Tail or Linker; Cycle is a fused aryl or heteroaryl group optionally substituted with 1 or 2 substituents independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, 15 wherein Cycle-A is optionally substituted with 1 or 2 substituents independently selected from R^{5a}; Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1 or 2 substituents independently selected from

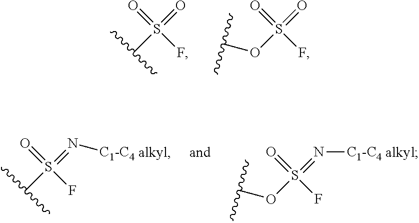

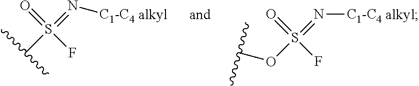

each R¹⁰, Rioa, and R^{10b} is independently selected from hydrogen, alkyl, haloalkyl, alkenyl, alkynyl, halogen, aryl, heteroaryl, heterocycle, cyano, nitro, —NR¹¹R¹³, —OR¹¹, -SR″, —C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R¹⁵;
each R¹⁴ is independently selected from hydrogen, alkyl, haloalkyl, alkenyl, alkynyl, 15 aryl, heteroaryl, heterocycle, amino, hydroxyl, alkoxy, —N(H)(alkyl), and —N(alkyl)₂; and each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂. 70. The compound of embodiment 69 wherein the compound is of Formula:

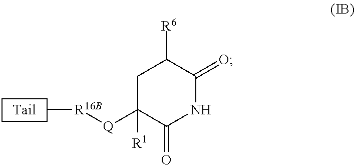

or a pharmaceutically acceptable salt thereof. 71. The compound of embodiment 69 wherein the compound is of Formula:

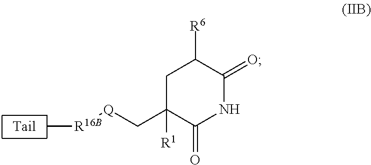

or a pharmaceutically acceptable salt thereof. 72. The compound of any one of embodiments 68-71, wherein Q is NH. 73. The compound of any one of embodiments 68-71, wherein Q is NCH₃. 74. The compound of any one of embodiments 68-71, wherein Q is CH₂. 75. The compound of any one of embodiments 68-71, wherein Q is O. 76. The compound of any one of embodiments 68-71, wherein Q is S. 77. The compound of embodiment 68 wherein the compound is of Formula:

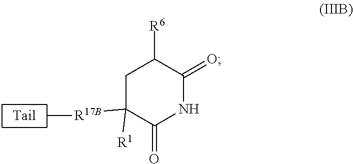

### R⁶ Tal R7B NH o (IIIB);

or a pharmaceutically acceptable salt thereof^{PGP}-4⁶⁰,C¹ 78. The compound of any one of embodiments 68-77, wherein R^{16B} and R^{17B} are

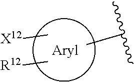

optionally substituted with 1, 2, or 3 substituents independently selected from R⁵. 79. The compound of any one of embodiments 68-77, wherein R^{16B} and R^{17B} are X12 Heteroaryl R12

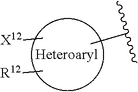

optionally substituted with 1, 2, or 3 substituents independently selected from R⁵. 80. The compound of any one of embodiments 68-77, wherein R^{16B} and R^{17B} are

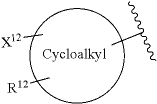

optionally substituted with 1, 2, or 3 substituents independently selected from R⁵.

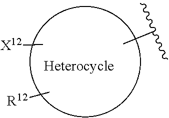

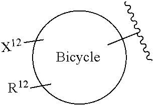

83. The compound of embodiment 1 wherein the compound is of Formula:

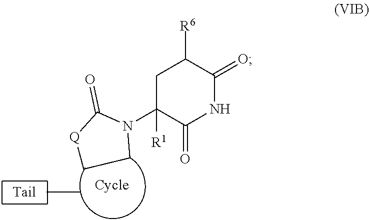

or a pharmaceutically acceptable salt thereof. 84. The compound of embodiment 83, wherein Cycle is phenyl optionally substituted with 1, 2, or 3 substituents independently selected from R⁵ and substituted with one R12 substituent. 85. The compound of any one of embodiments 83-84, wherein Q is NH. 86. The compound of any one of embodiments 83-84, wherein Q is NCH₃. 87. The compound of any one of embodiments 83-84, wherein Q is CH₂. 88. The compound of any one of embodiments 83-84, wherein Q is O. 89. The compound of any one of embodiments 83-84, wherein Q is S. 90. The compound of embodiment 1 wherein the compound is of Formula:

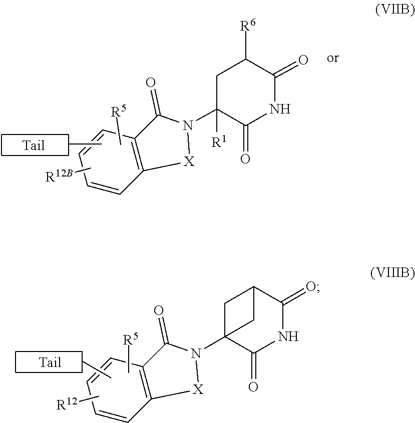

or a pharmaceutically acceptable salt thereof. 91. The compound of embodiment 90, wherein X is CH₂. 92. The compound of embodiment 90, wherein X is C(O). 93. The compound of any one of embodiments 68-92, wherein R¹ is hydrogen. 94. The compound of any one of embodiments 68-92, wherein R¹ is CH₃. 95. The compound of any one of embodiments 68-92, wherein R¹ and R⁶ combined form a one-carbon bridge. 96. The compound of any one of embodiments 68-94, wherein R⁶ is hydrogen. 97. The compound of any one of embodiments 68-94, wherein R⁶ is CH₃. 98. The compound of embodiment 1 wherein the compound is of Formula:

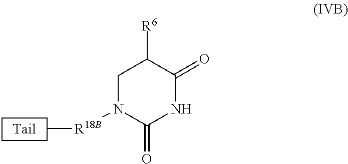

or a pharmaceutically acceptable salt thereof. 99. The compound of embodiment 98, wherein R⁶ is hydrogen. 100. The compound of embodiment 98, wherein R⁶ is CH₃. 101. The compound of any one of embodiments 98-100, wherein R^{8B} is X12 Aryl

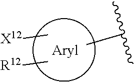

optionally substituted with 1 or 2 substituents independently selected from R⁵. 102. The compound of any one of embodiments 98-100, wherein R^{18B} is

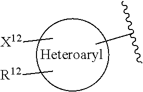

which is attached to the azaglutarimide moiety through a C—N bond and optionally substituted with 1 or 2 substituents independently selected from R⁵. 103. The compound of any one of embodiments 68-102, wherein each R⁵ is independently selected from hydrogen, alkyl, haloalkyl, and halogen. 104. The compound of any one of embodiments 68-102, wherein each R⁵ is hydrogen. 105. The compound of any one of embodiments 68-102, wherein one R⁵ is F. 106. The compound of any one of embodiments 68-102, wherein R⁵ is —NR⁷R⁸ or —OR⁷. 107. The compound of embodiment 106, wherein R⁷ is hydrogen. 108. The compound of embodiment 106, wherein R⁷ is methyl. 109. The compound of any one of embodiments 106-108, wherein R⁸ is hydrogen. 110. The compound of any one of embodiments 106-108, wherein R⁸ is methyl. 111. The compound of any one of embodiments 68-102, wherein R⁵ is aryl or heteroaryl optionally substituted with 1, 2, or 3 substituents independently selected from R¹⁰ . 112. The compound of any one of embodiments 68-102, wherein R⁵ is cyano. 113. The compound of any one of embodiments 68-102, wherein R⁵ is nitro. 114. The compound of any one of embodiments 68-102, wherein R⁵ -C(O)CH₃. 115. The compound of embodiment 68 wherein the compound is of Formula:

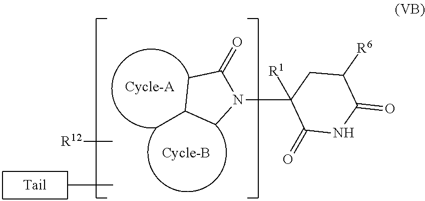

or a pharmaceutically acceptable salt thereof. 116. The compound of embodiment 115, wherein Cycle-A is phenyl optionally substituted with 1 or 2 substituents independently selected from Rs. 117. The compound of embodiment 115 or 116, wherein Cycle-B is phenyl optionally substituted with 1 or 2 substituents independently selected from R^{5b}. 118. The compound of any one of embodiments 115-117, wherein R¹ is hydrogen. 119. The compound of any one of embodiments 115-117, wherein R¹ is CH₃. 120. The compound of any one of embodiments 115-117, wherein R¹ and R⁶ combined form a one-carbon bridge. 121. The compound of any one of embodiments 115-119, wherein R⁶ is hydrogen. 122. The compound of any one of embodiments 115-119, wherein R⁶ is CH₃.

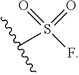 [SMILES: *S(=O)(=O)F]

123. The compound of any one of embodiments 68-122, wherein R¹² is F -124. The compound of any one of embodiments 68-122, wherein R¹² is O F

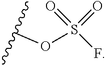 [SMILES: *OS(=O)(=O)F]

125. The compound of any one of embodiments 68-122, wherein R¹² is

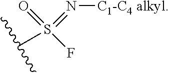

126. The compound of any one of embodiments 68-122, wherein R¹² is

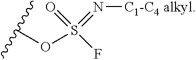

127. The compound of any one of embodiments 68-126 wherein Tail is selected from:

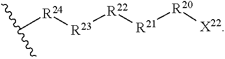

128. The compound of any one of embodiments 68-127, wherein R²⁰ is bond.

129. The compound of any one of embodiments 68-127, wherein R²⁰ is CH2.

130. The compound of any one of embodiments 68-127, wherein R²⁰ is heterocycle.

131. The compound of any one of embodiments 68-127, wherein R²⁰ is aryl.

132. The compound of any one of embodiments 68-127, wherein R²⁰ is phenyl.

133. The compound of any one of embodiments 68-127, wherein R²⁰ is bicycle.

134. The compound of any one of embodiments 68-133, wherein R²¹ is bond.

135. The compound of any one of embodiments 68-133, wherein R²¹ is CH2.

136. The compound of any one of embodiments 68-133, wherein R²¹ is heterocycle.

137. The compound of any one of embodiments 68-133, wherein R²¹ is aryl.

138. The compound of any one of embodiments 68-133, wherein R²¹ is phenyl.

139. The compound of any one of embodiments 68-133, wherein R²¹ is bicycle.

140. The compound of any one of embodiments 68-139, wherein R²² is bond.

141. The compound of any one of embodiments 68-139, wherein R²² is CH2.

142. The compound of any one of embodiments 68-139, wherein R²² is heterocycle.

143. The compound of any one of embodiments 68-139, wherein R²² is aryl.

144. The compound of any one of embodiments 68-139, wherein R²² is phenyl.

145. The compound of any one of embodiments 68-139, wherein R²² is bicycle.

146. The compound of any one of embodiments 68-145, wherein R²³ is bond.

147. The compound of any one of embodiments 68-145, wherein R²³ is CH2.

148. The compound of any one of embodiments 68-145, wherein R²³ is heterocycle.

149. The compound of any one of embodiments 68-145, wherein R²³ is aryl.

150. The compound of any one of embodiments 68-145, wherein R²³ is phenyl.

151. The compound of any one of embodiments 68-145, wherein R²³ is bicycle.

152. The compound of any one of embodiments 68-151, wherein R²⁴ is bond.

153. The compound of any one of embodiments 68-151, wherein R²⁴ is CH2.

154. The compound of any one of embodiments 68-151, wherein R²⁴ is heterocycle.

155. The compound of any one of embodiments 68-151, wherein R²⁴ is aryl.

156. The compound of any one of embodiments 68-151, wherein R²⁴ is phenyl.

157. The compound of any one of embodiments 68-151, wherein R²⁴ is bicycle.

158. The compound of any one of embodiments 68-151, wherein R²⁴ is C(O).

159. The compound of any one of embodiments 68-126 wherein Tail is selected from

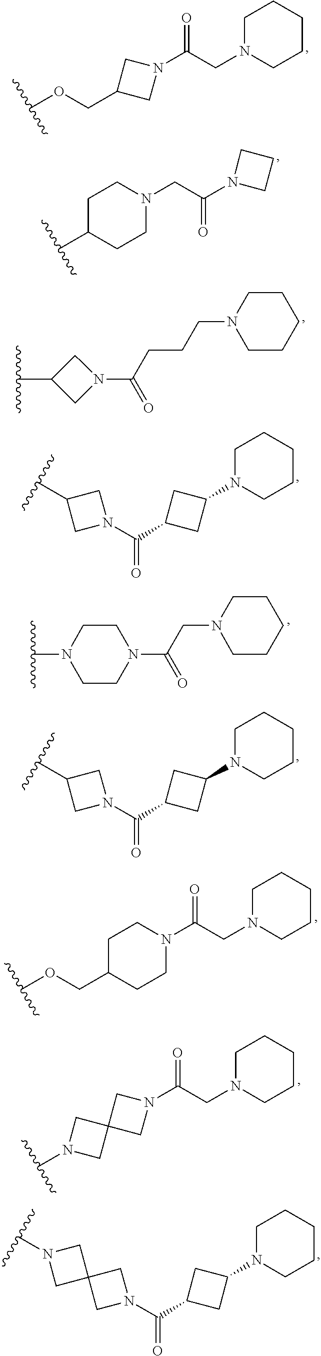

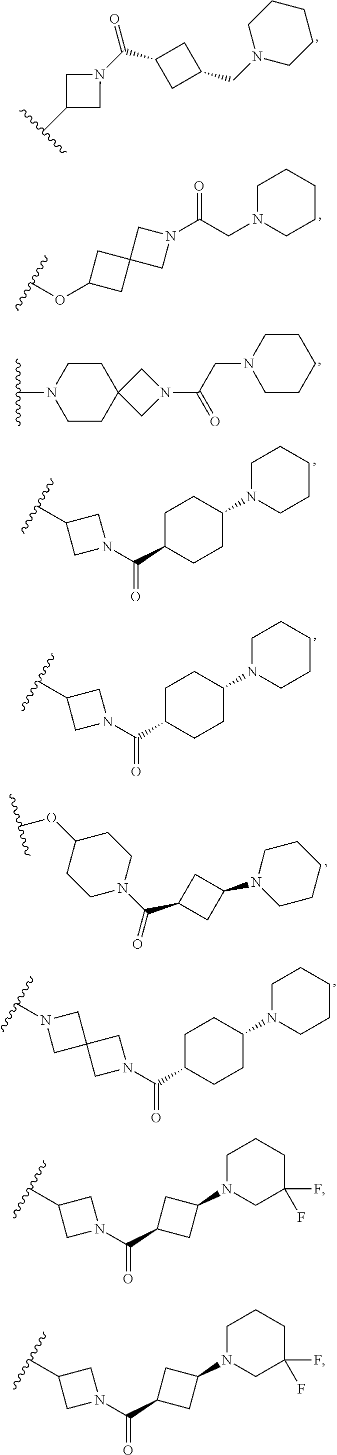

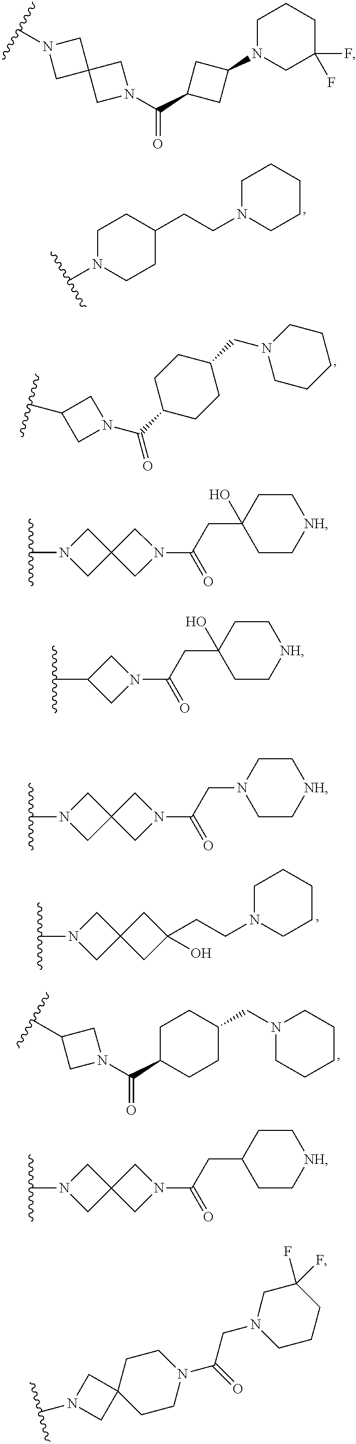

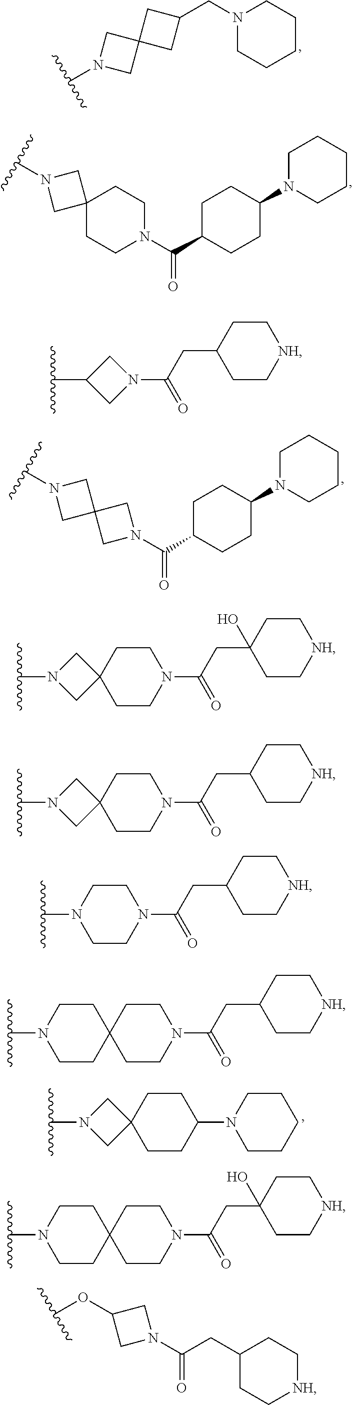

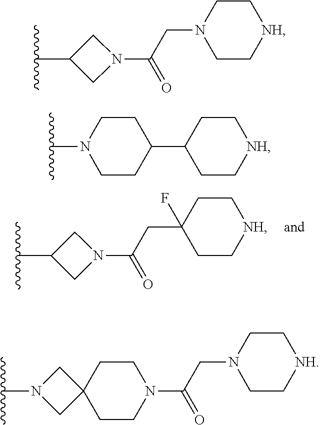

160. The compound of any one of embodiments 68-126 wherein Tail is selected^{PGP}-4⁷⁷,C²,M

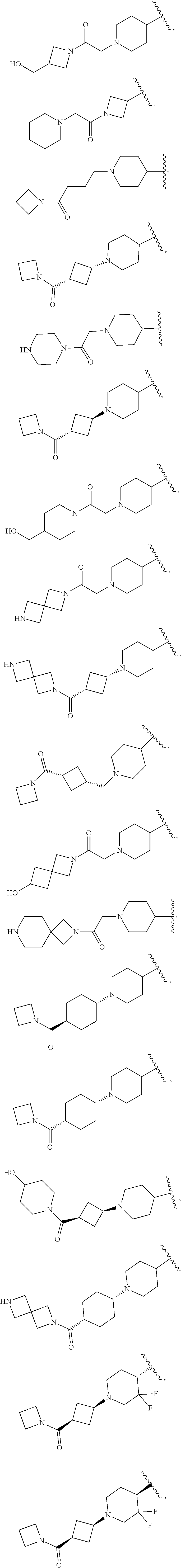

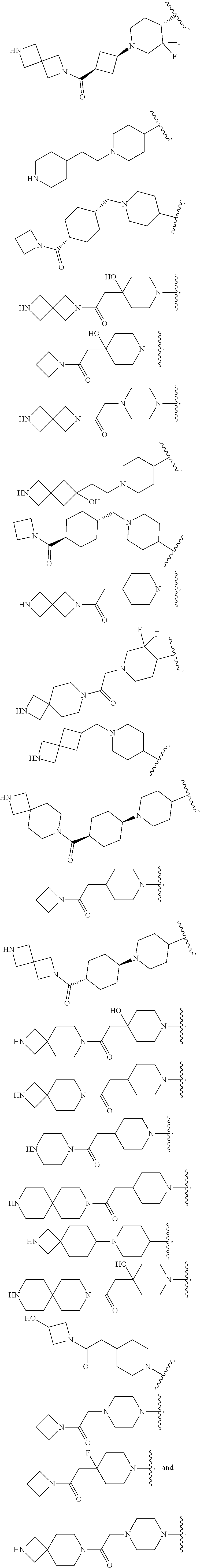

161. A pharmaceutical composition comprising a compound of any one of^{PGP}-4⁷⁸,C²,M embodiments 1-160 or a pharmaceutically acceptable salt thereof and a pharmaceutically acceptable carrier. 162. A method of treating a disorder comprising administering an effective amount of a compound of any one of embodiments 1-160 or a pharmaceutical composition of embodiment 161, wherein the disorder is mediated by a protein that is degraded or downregulated by the compound. 163. The method of embodiment 162, wherein the protein is a zinc finger protein. 164. The method of embodiment 162, wherein the protein is selected from: ARID2, CDK1, CDK12, CDK13, CK1alpha, CSNK1A1, Cyclin K, E4F1, FAM83F, GSPT1, GSPT2, GZF1, IKZF1, IKZF2, IKZF3, IKZF4, ILF2, Myc, ODC1, p63, PDE6D, AB28, RARalpha-ZBTB16, RBM23, RBM39, RBM39, RNF166, SALL4, WBP4, ZBTB16, ZBTB16-RARalpha, ZBTB39, ZFP91, ZFP91, ZFP91, ZMYM2-FGFR1, ZMYM2-FLT3, ZNF198, ZNF276, ZNF276, ZNF517, ZNF582, ZNF653, ZNF654, ZNF692, ZNF787, ZNF827, and ZNF98. 165. The method of embodiment 162, wherein the protein is selected from: ARID2, aromatase, CDK12, NRF2, PDE6D, CK1alpha, cyclin K, GSPT1, FAM83, ILF2, ZBTB16, and ZMYM2. 166. A compound of Formula:

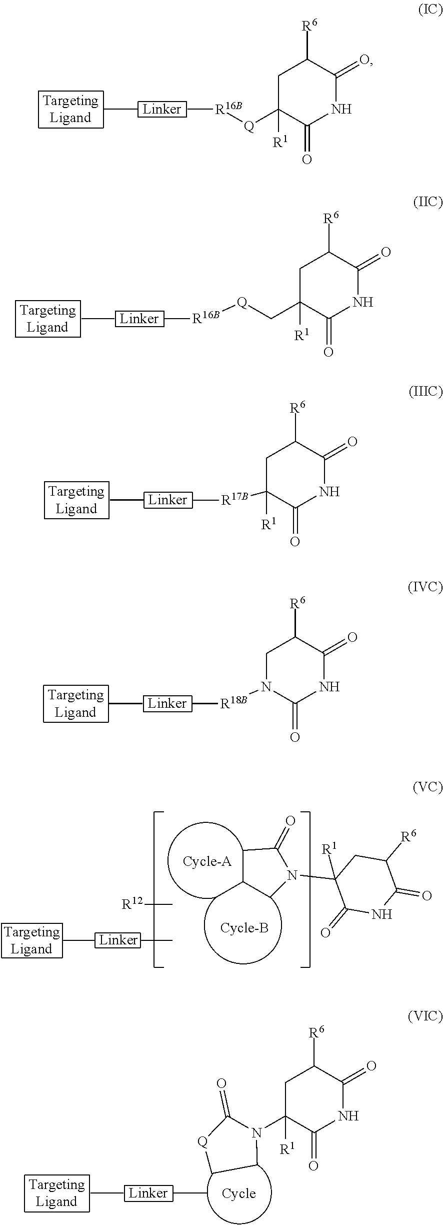

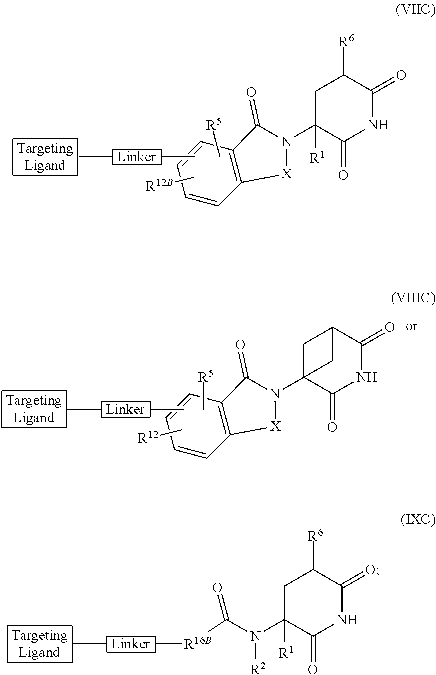

or a pharmaceutically acceptable salt thereof, wherein: Q is CH₂, NR², O, or S; X is CH₂ orC( ) R¹ and R⁶ are independently selected from hydrogen, alkyl, alkenyl, alkynyl, and 10 halogen;
or R¹ and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from Rioa; Linker is of Foripula

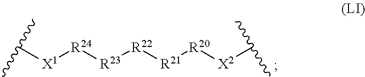

X¹ and X² are independently at each occurrence selected from bond, carbamate, heterocycle, NR⁴², C(R⁴²)2, O, C(O), and S; R⁴² is independently at each occurrence selected from the group consisting of hydrogen, alkyl, heteroalkyl, heterocycle, aryl, heteroaryl, —C(O)H, —C(O)OH, —C(O)alkyl, —C(O)Oalkyl, —C(O)(aryl, heteroalkyl or heteroaryl), —C(O)O(aryl, heteroalkyl, or heteroaryl), alkene, and alkyne; R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, 15 —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, heteroaryl, and carbocycle; each of which is optionally substituted with 1, 2, or 3 substituents independently selected from R⁴⁰; R²⁶ is independently at each occurrence selected from the group consisting of hydrogen, alkyl, arylalkyl, heteroarylalkyl, alkene, alkyne, aryl, heteroaryl, and heterocycle; R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkene, and alkyne; R⁴⁰ is independently at each occurrence selected from the group consisting of hydrogen, alkyl, alkene, alkyne, fluoro, bromo, chloro, azide, OR⁷, NR⁷R⁸, S(O)NR⁷R⁸, S(O)2NR⁷R⁸, 25 S(O)OR⁷, S(O)₂OR⁷, haloalkyl, aryl, heteroaryl, heterocycle, and cycloalkyl; R⁴¹ is independently at each occurrence selected from the group consisting of hydrogen, alkyl, alkene, alkyne, haloalkyl, aryl, heteroaryl, heterocycle, and cycloalkyl;

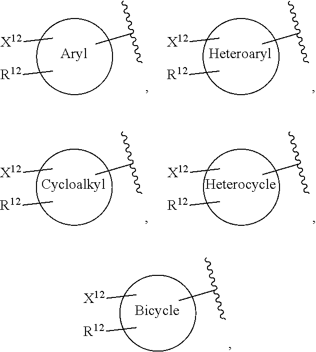

each of which is optionally substituted with 1 or 2 substituents independently selected from R⁵; R¹⁷B is selected from:

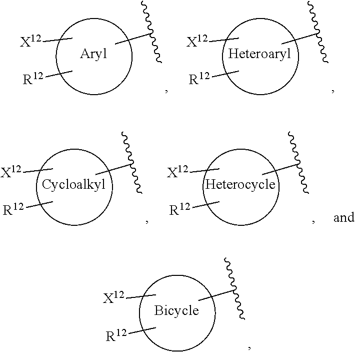

and, each of which is optionally substituted with 1 or 2 substituents independently selected from R⁵; R^{18B} is selected from: X12 X12 X12 Aryl Heteroaryl Cycloalkyl 012R12 R12

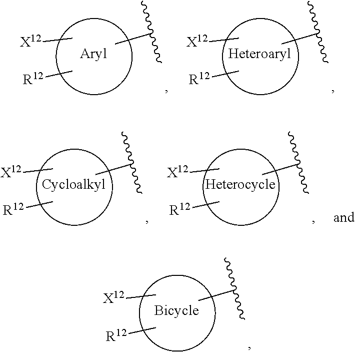

R^{2 hu l} and each of which is attached to the azaglutarimide moiety through a C—N bond and each of which R^{18B} is optionally substituted with 1 or 2 substituents independently selected from R⁵; X¹² is the attachment point to Tail or Linker; Cycle is a fused aryl or heteroaryl group optionally substituted with 1 or 2 substituents independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a}.

Cycle-B is a fused ring selected from phenyl, 5- or 6-membered heteroaryl, 5- to 8-membered heterocycle, 5- to 8-membered cycloalkyl, or 5- to 8-membered cycloalkenyl, 5 wherein Cycle-B is optionally substituted with 1 or 2 substituents independently selected from

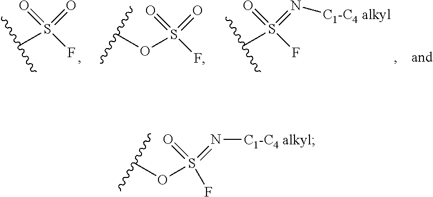

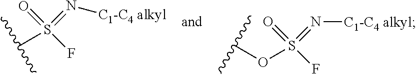

each R¹⁰, R^{10a}, and R^{10b} is independently selected from hydrogen, alkyl, haloalkyl, 15 alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 20 2 substituents independently selected from R¹⁵;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; and each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and 25 —N(alkyl)₂. 167. The compound of embodiment 166 wherein the compound is of Formula:

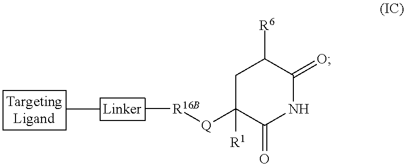

or a pharmaceutically acceptable salt thereof. 168. The compound of embodiment 166 wherein the compound is of Formula:

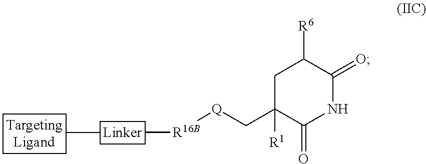

or a pharmaceutically acceptable salt thereof. 169. The compound of any one of embodiments 166-168, wherein Q is NH. 170. The compound of any one of embodiments 166-168, wherein Q is NCH₃. 171. The compound of any one of embodiments 166-168, wherein Q is CH₂. 172. The compound of any one of embodiments 166-168, wherein Q is O. 173. The compound of any one of embodiments 166-168, wherein Q is S. 174. The compound of embodiment 166 wherein the compound is of Formula:

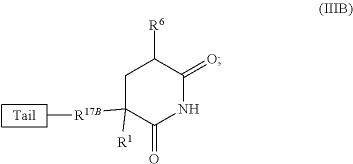

or a pharmaceutically acceptable salt thereof. 175. The compound of any one of embodiments 166-174, wherein R^{16B} and R^{17B} are

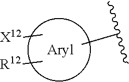

ptionally substituted with 1, 2, or 3 substituents independently selected from R⁵. 176. The compound of any one of embodiments 166-174, wherein R″ ^{6B} and R^{7B} are

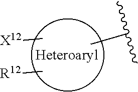

optionally substituted with 1, 2, or 3 substituents independently selected from R⁵. 177. The compound of any one of embodiments 166-174, wherein R^{16B} and R^{17B} are X12 Cycloalkyl

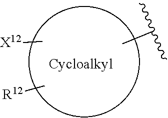

R^{12 hu l} optionally substituted with 1, 2, or 3 substituents independently selected from R⁵. 178. The compound of any one of embodiments 166-174, wherein R^{16B} and R^{17B} are Heterocycle

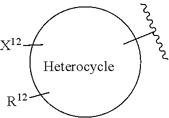

R^{12 hu l} optionally substituted with 1, 2, or 3 substituents independently selected from R⁵. 179. The compound of any one of embodiments 166-174, wherein R^{16B} and R^{17B} are

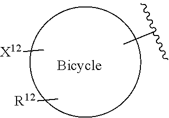

ioptionally substituted with 1, 2, or 3 substituents independently selected from R⁵. 180. The compound of embodiment 166 wherein the compound is of Formula:

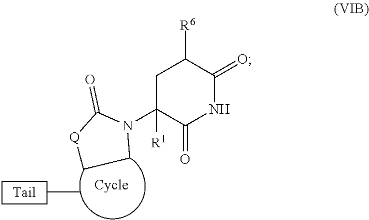

### Rs O O Tail Cycle (VIB);

15 or a pharmaceutically acceptable salt thereof. 181. The compound of embodiment 180, wherein Cycle is phenyl optionally substituted with 1, 2, or 3 substituents independently selected from R⁵ and substituted with one R¹² substituent. 182. The compound of any one of embodiments 180-181, wherein Q is NH. 183. The compound of any one of embodiments 180-181, wherein Q is NCH₃. 184. The compound of any one of embodiments 180-181, wherein Q is CH₂. 185. The compound of any one of embodiments 180-181, wherein Q is O. 186. The compound of any one of embodiments 180-181, wherein Q is S. 187. The compound of embodiment 1 wherein the compound is of Formula:

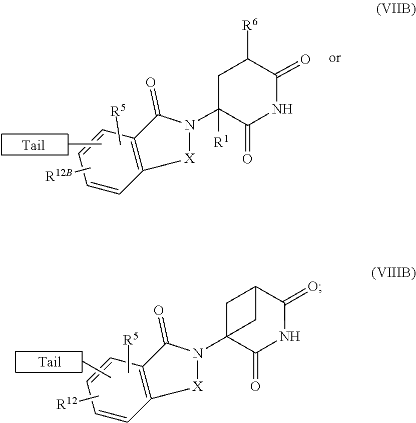

or a pharmaceutically acceptable salt thereof. 188. The compound of embodiment 187, wherein X is CH₂. 189. The compound of embodiment 187, wherein X is C(O). 190. The compound of any one of embodiments 166-189, wherein R¹ is hydrogen. 191. The compound of any one of embodiments 166-189, wherein R¹ is CH₃. 192. The compound of any one of embodiments 166-189, wherein R¹ and R⁶ combined form a one-carbon bridge. 193. The compound of any one of embodiments 166-191, wherein R⁶ is hydrogen. 194. The compound of any one of embodiments 166-191, wherein R⁶ is CH₃. 195. The compound of embodiment 166 wherein the compound is of Formula:

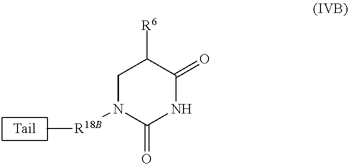

or a pharmaceutically acceptable salt thereof. 196. The compound of embodiment 195, wherein R⁶ is hydrogen. 197. The compound of embodiment 195, wherein R⁶ is CH₃. 198. The compound of any one of embodiments 195-197, wherein R^{18B} is

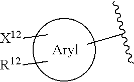

optionally substituted with 1 or 2 substituents independently selected from R⁵. 199. The compound of any one of embodiments 195-197, wherein R^{18B} is

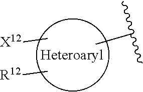

which is attached to the azaglutarimide moiety through a C—N bond and optionally substituted with 1 or 2 substituents independently selected from R⁵. 200. The compound of any one of embodiments 166-199, wherein each R⁵ is independently selected from hydrogen, alkyl, haloalkyl, and halogen. 201. The compound of any one of embodiments 166-199, wherein each R⁵ is hydrogen. 202. The compound of any one of embodiments 166-199, wherein one R⁵ is F. 203. The compound of any one of embodiments 166-199, wherein R⁵ is —NR⁷R or —OR⁷. 204. The compound of embodiment 203, wherein R⁷ is hydrogen. 205. The compound of embodiment 203, wherein R⁷ is methyl. 206. The compound of any one of embodiments 203-205, wherein R is hydrogen. 207. The compound of any one of embodiments 203-205, wherein R is methyl. 208. The compound of any one of embodiments 166-199, wherein R⁵ is aryl or heteroaryl optionally substituted with 1, 2, or 3 substituents independently selected from R¹⁰ . 209. The compound of any one of embodiments 166-199, wherein R⁵ is cyano. 210. The compound of any one of embodiments 166-199, wherein R⁵ is nitro. 211. The compound of any one of embodiments 166-199, wherein R⁵ -C(O)CH₃. 212. The compound of embodiment 166 wherein the compound is of Formula:

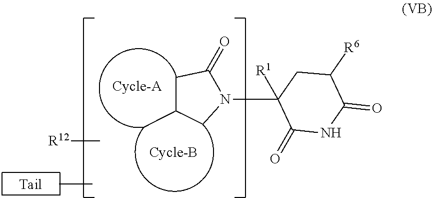

or a pharmaceutically acceptable salt thereof. 213. The compound of embodiment 212, wherein Cycle-A is phenyl optionally substituted with 1 or 2 substituents independently selected from Rsa 214. The compound of embodiment 212 or 213, wherein Cycle-B is phenyl optionally substituted with 1 or 2 substituents independently selected from R^{5b}. 215. The compound of any one of embodiments 212-214, wherein R¹ is hydrogen. 216. The compound of any one of embodiments 212-214, wherein R¹ is CH₃. 217. The compound of any one of embodiments 212-214, wherein R¹ and R⁶ combined form a one-carbon bridge. 218. The compound of any one of embodiments 212-216, wherein R⁶ is hydrogen. 219. The compound of any one of embodiments 212-216, wherein R⁶ is CH₃.

220. The compound of any one of embodiments 166-219, wherein R¹² is F

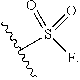 [SMILES: *S(=O)(=O)F]

221. The compound of any one of embodiments 166-219, wherein R¹² i O F

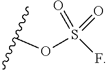 [SMILES: *OS(=O)(=O)F]

222. The compound of any one of embodiments 166-219, wherein R¹² is

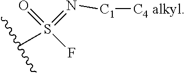

223. The compound of any one of embodiments 166-219, wherein R¹² is

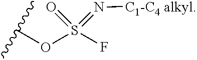

226. The compound of any one of embodiments 166-224, wherein X¹ is heterocycle.

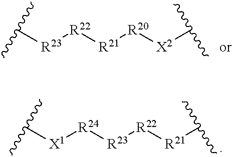

227. The compound of any one of embodiments 166-224, wherein X¹ is NR². 228. The compound of any one of embodiments 166-224, wherein X¹ is C(O). 229. The compound of any one of embodiments 166-228, wherein X² is bond. 230. The compound of any one of embodiments 166-224, wherein X² is heterocycle. 231. The compound of any one of embodiments 166-224, wherein X² is NR². 232. The compound of any one of embodiments 166-224, wherein X² is C(O). 233. The compound of any one of embodiments 166-232, wherein R²⁰ is bond. 234. The compound of any one of embodiments 166-232, wherein R²⁰ is CH₂. 235. The compound of any one of embodiments 166-232, wherein R²⁰ is heterocycle. 236. The compound of any one of embodiments 166-232, wherein R²⁰ is aryl. 237. The compound of any one of embodiments 166-232, wherein R²⁰ is phenyl. 238. The compound of any one of embodiments 166-232, wherein R²⁰ is bicycle. 239. The compound of any one of embodiments 166-238, wherein R²¹ is bond. 240. The compound of any one of embodiments 166-238, wherein R²¹ is CH₂. 241. The compound of any one of embodiments 166-238, wherein R²¹ is heterocycle. 242. The compound of any one of embodiments 166-238, wherein R²¹ is aryl. 243. The compound of any one of embodiments 166-238, wherein R²¹ is phenyl. 244. The compound of any one of embodiments 166-238, wherein R²¹ is bicycle. 245. The compound of any one of embodiments 166-223, wherein Linker is of

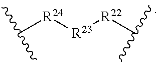

246. The compound of any one of embodiments 166-245, wherein R²² is bond. 247. The compound of any one of embodiments 166-245, wherein R²² is CH₂. 248. The compound of any one of embodiments 166-245, wherein R²² is heterocycle. 249. The compound of any one of embodiments 166-245, wherein R²² is aryl. 250. The compound of any one of embodiments 166-245, wherein R²² is phenyl. 251. The compound of any one of embodiments 166-245, wherein R²² is bicycle. 252. The compound of any one of embodiments 166-223, wherein Linker is of

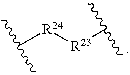

253. The compound of any one of embodiments 166-252, wherein R²³ is bond. 254. The compound of any one of embodiments 166-252, wherein R²³ is CH₂. 255. The compound of any one of embodiments 166-252, wherein R²³ is heterocycle. 256. The compound of any one of embodiments 166-252, wherein R²³ is aryl. 257. The compound of any one of embodiments 166-252, wherein R²³ is phenyl. 258. The compound of any one of embodiments 166-252, wherein R²³ is bicycle. 259. The compound of any one of embodiments 166-223, wherein Linker is of formula:

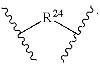

260. The compound of any one of embodiments 166-259, wherein R²⁴ is bond. 261. The compound of any one of embodiments 166-259, wherein R²⁴ is CH₂. 262. The compound of any one of embodiments 166-259, wherein R²⁴ is heterocycle. 263. The compound of any one of embodiments 166-259, wherein R²⁴ is aryl. 264. The compound of any one of embodiments 166-259, wherein R²⁴ is phenyl. 265. The compound of any one of embodiments 166-259, wherein R²⁴ is bicycle. 266. The compound of any one of embodiments 166-259, wherein R²⁴ is C(O). 267. The compound of any one of embodiments 166-223, wherein the Linker is selected from the group consisting of:

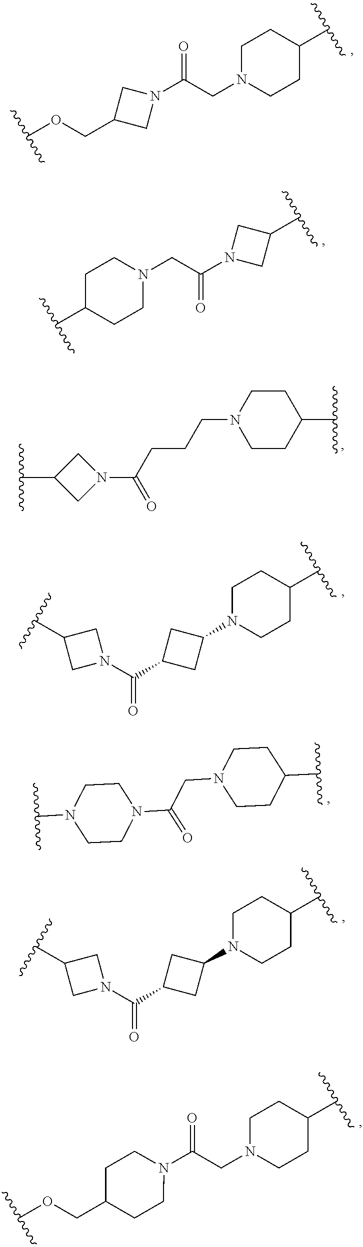

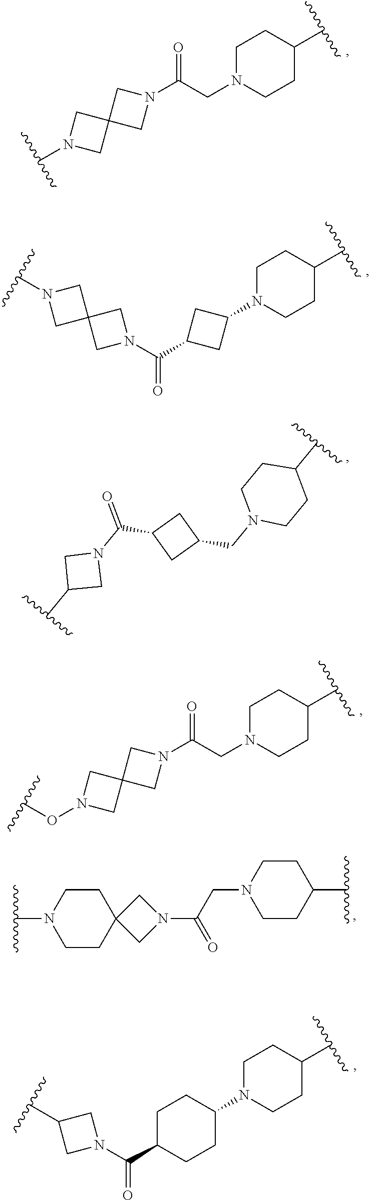 [SMILES: *C1CCN(CC(=O)N2CC3(CN(*)C3)C2)CC1 ; *C1CCN([C@H]2C[C@@H](C(=O)N3CC4(CN(*)C4)C3)C2)CC1 ; *C1CCN(C[C@H]2C[C@@H](C(=O)N3CC(*)C3)C2)CC1 ; *ON1CC2(C1)CN(C(=O)CN1CCC(*)CC1)C2 ; *C1CCN(CC(=O)N2CC3(CCN(*)CC3)C2)CC1 ; *C1CCN([C@H]2CC[C@H](C(=O)N3CC(*)C3)CC2)CC1]

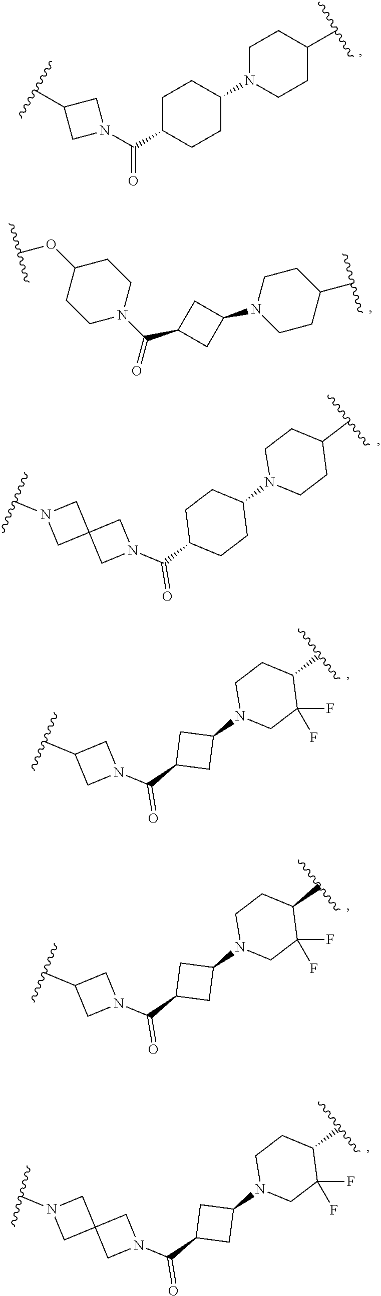 [SMILES: *C1CCN([C@H]2CC[C@@H](C(=O)N3CC(*)C3)CC2)CC1 ; *OC1CCN(C(=O)[C@H]2C[C@@H](N3CCC(*)CC3)C2)CC1 ; *C1CCN([C@H]2CC[C@@H](C(=O)N3CC4(CN(*)C4)C3)CC2)CC1 ; *C1CN(C(=O)[C@H]2C[C@@H](N3CC[C@H](*)C(F)(F)C3)C2)C1 ; *C1CN(C(=O)[C@H]2C[C@@H](N3CC[C@@H](*)C(F)(F)C3)C2)C1 ; *[C@H]1CCN([C@H]2C[C@@H](C(=O)N3CC4(CN(*)C4)C3)C2)CC1(F)F]

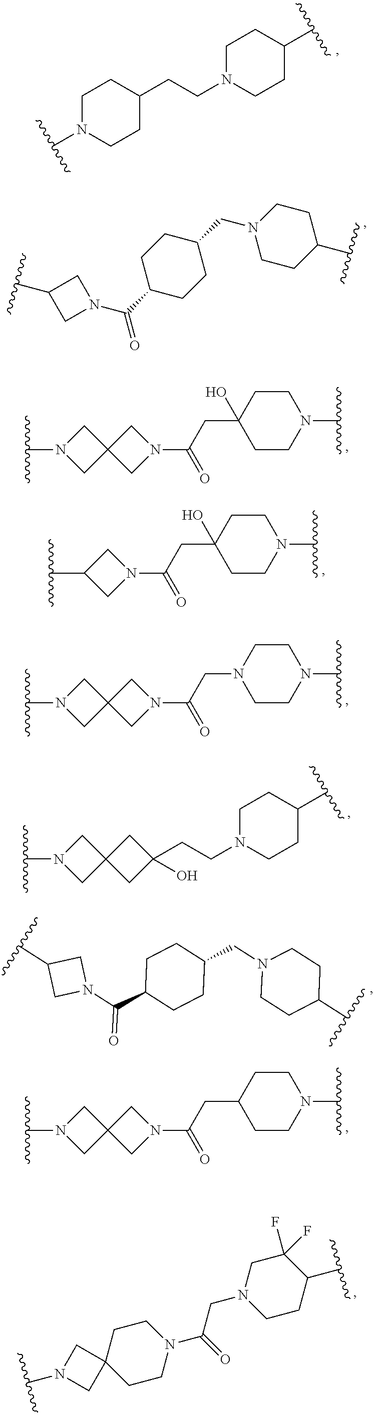

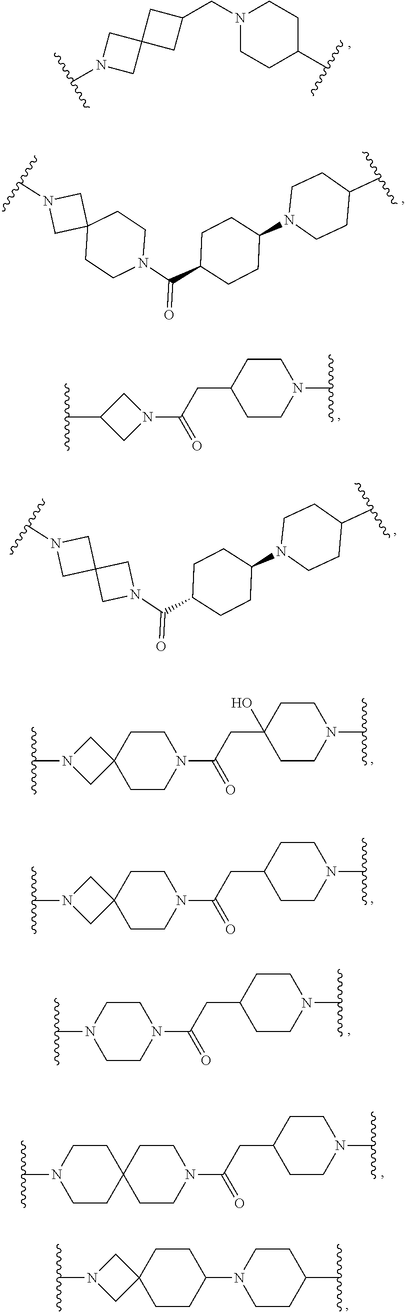

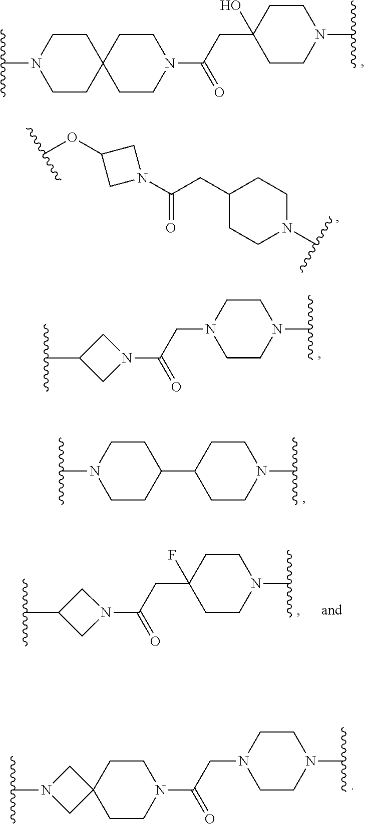 [SMILES: *N1CCC(O)(CC(=O)N2CCC3(CCN(*)CC3)CC2)CC1 ; *OC1CN(C(=O)CC2CCN(*)CC2)C1 ; *C1CN(C(=O)CN2CCN(*)CC2)C1 ; *N1CCC(C2CCN(*)CC2)CC1 ; *C1CN(C(=O)CC2(F)CCN(*)CC2)C1 ; *N1CCN(CC(=O)N2CCC3(CC2)CN(*)C3)CC1]

268. A pharmaceutical composition comprising a compound of any one of embodiments 166-267 or a pharmaceutically acceptable salt thereof and a pharmaceutically acceptable carrier. 269. A method of treating a disorder that is mediated by the Target Protein in a patient in need thereof comprising administering an effective amount of a compound of any one of embodiments 166-267 or a pharmaceutical composition of embodiment 268.

Additional Embodiments of the Invention A1.A compound of Formula:

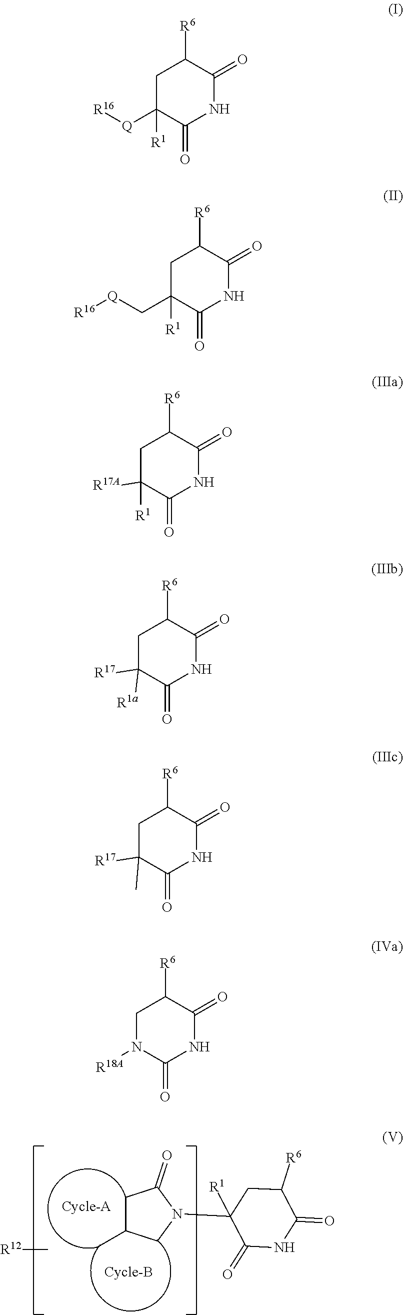

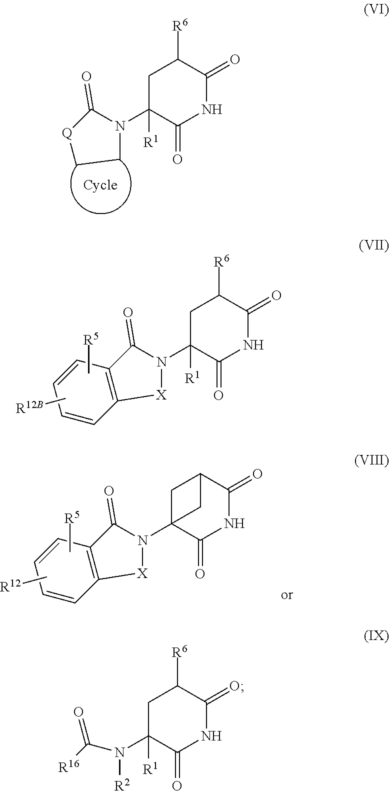

or a pharmaceutically acceptable salt thereof; wherein: Q is CH₂, NR2, O, or S; X is CH₂ or C(O); R¹ and R⁶ are independently selected from hydrogen, alkyl, alkenyl, alkynyl, and 10 halogen;
or R¹ and R⁶ are combined to form a one or two carbon bridge to form a fused cycle; R^{1a} is independently selected from hydrogen, C₃-C₈ alkyl, alkenyl, alkynyl, and halogen;
or R^{1a} and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R⁵¹ is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a}; R⁵⁰ is independently selected from alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, 10 -S(O)₂R⁹, and —P(O)(R⁹)₂; each of which except hydrogen, halogen, cyano, and nitro is optionally substituted with 1 or 2 substituents independently selected from R^{10a}; R¹⁶ is selected from:

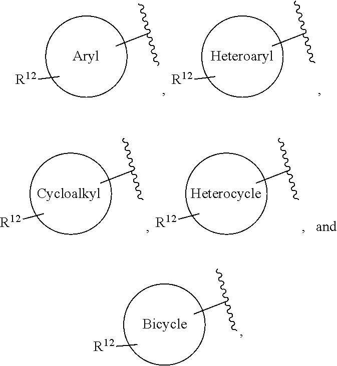

and,each of which is optionally substituted with 1 or 2 substituents 15 independently selected from R⁵; R¹⁷ is selected from:

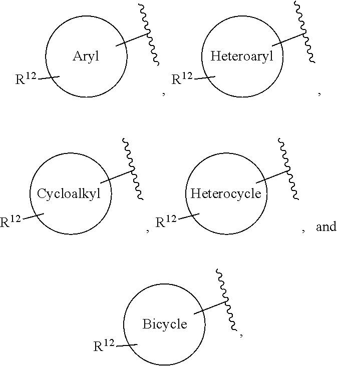

and, each of which is optionally substituted with 1 or 2 substituents independently selected from R⁵; R¹¹ is selected from:

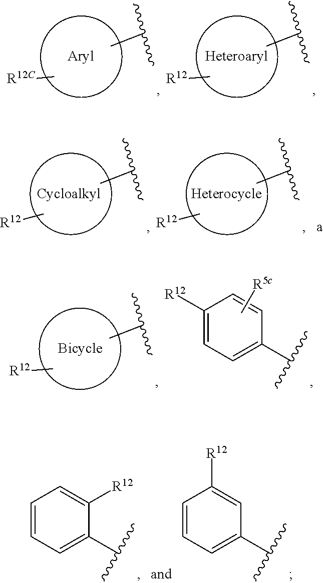

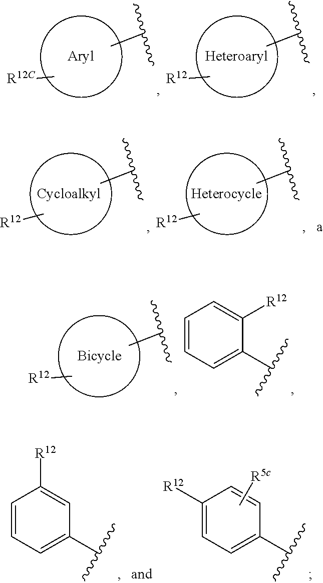

of which is attached to the azaglutarimide moiety through a C—N bond and each of which R¹⁸A is optionally substituted with 1 or 2 substituents independently selected from R⁵; Cycle is a fused aryl or heteroaryl group optionally substituted with 1 or 2 substituents independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from 15 R^{5a}; Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1 or 2 substituents independently selected from R^{5b} O.0 0 0,N-C₁-C₄ alkyl O, N—C₁-C₄ alkyl S/ ,I L R¹² is selected from F F, F,and 0 F

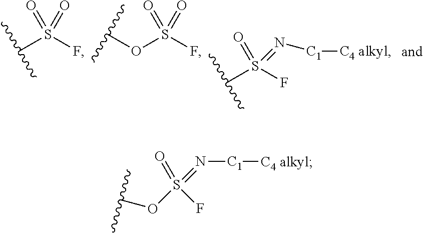

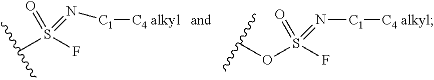

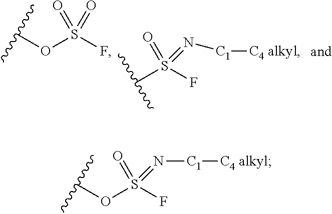

R⁷ and R⁸ at each instance are independently selected from hydrogen, alkyl, haloalkyl, alkenyl, alkynyl, aryl, heteroaryl, heterocycle, and C(O)R¹⁴ each of which except hydrogen is 5 optionally substituted with 1 or 2 substituents independently selected from R^{10b}each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R¹oa, and R1o^{b} is independently selected from hydrogen, alkyl, haloalkyl, alkenyl, alkynyl, halogen, aryl, heteroaryl, heterocycle, cyano, nitro, —NR¹¹R¹³, —OR¹¹, —SR¹¹, 10 -C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R¹⁵;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; and each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂.

A2. The compound of embodiment A1 wherein the compound is of Formula: R6 -O R¹⁶ NH QW

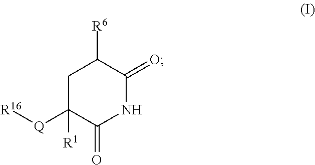

0 (1);
or a pharmaceutically acceptable salt thereof^{PGP}-5¹⁸,C¹ A3. The compound of embodiment A1 wherein the compound is of Formula:

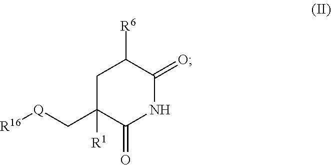

or a pharmaceutically acceptable salt thereof.

A4. The compound of any one of embodiments A1-A3, wherein Q is NH.

A5. The compound of any one of embodiments A1-A3, wherein Q is NCH₃.

A6. The compound of any one of embodiments A1-A3, wherein Q is CH₂.

A7. The compound of any one of embodiments A1-A3, wherein Q is O.

A8. The compound of any one of embodiments A1-A3, wherein Q is S.

A9. The compound of embodiment A1 wherein the compound is of Formula: O

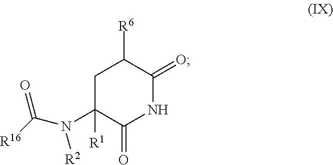

or a pharmaceutically acceptable salt thereof.

A10. The compound of embodiment A9, wherein R² is hydrogen.

A1 1. The compound of embodiment A9, wherein R² is methyl.

A12. The compound of embodiment A9, wherein R² is alkyl optionally substituted with 1 or 2 substituents independently selected from R¹⁰.

A13. The compound of embodiment A1 wherein the compound is of Formula:

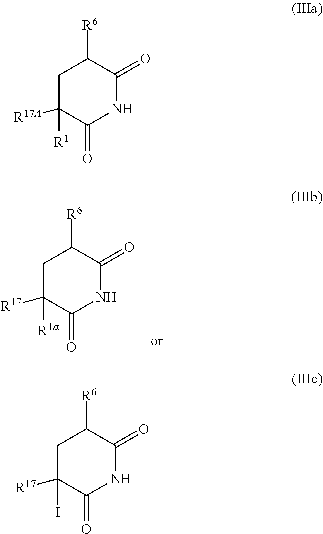

or a pharmaceutically acceptable salt thereof. A14. The compound of embodiment A1 wherein the compound is of Formula:

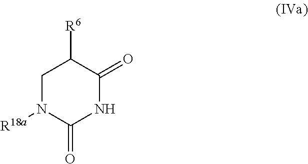

### R6 N NH R18A O (IVa)

or a pharmaceutically acceptable salt thereof. A15. The compound of any one of embodiments A1-A13, wherein R¹⁶ and R¹⁷ are

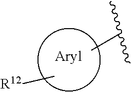

optionally substituted with 1 or 2 substituents independently selected from R⁵.

A16. The compound of embodiment A13 or embodiment A14, wherein R1^{7A} and R¹⁸A

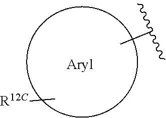

are optionally substituted with 1 or 2 substituents independently selected from R⁵.

A17. The compound of any one of embodiments A1-A13, wherein R¹⁶ and R¹⁷ are selected from the group consisting of:

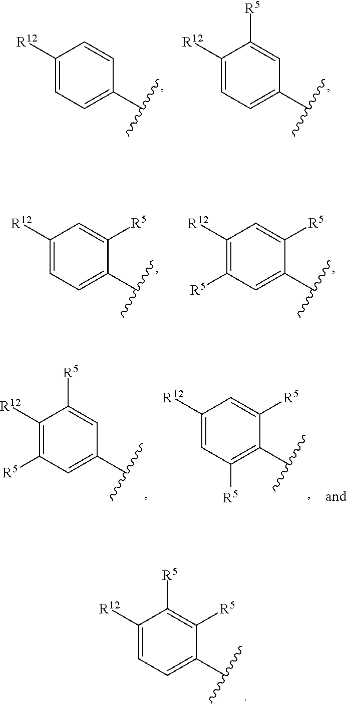

A18. The compound of any one of embodiments A1-A14, wherein R¹⁶, R¹⁷, R^{l7}A and R^{18A} are selected from the group consisting of:

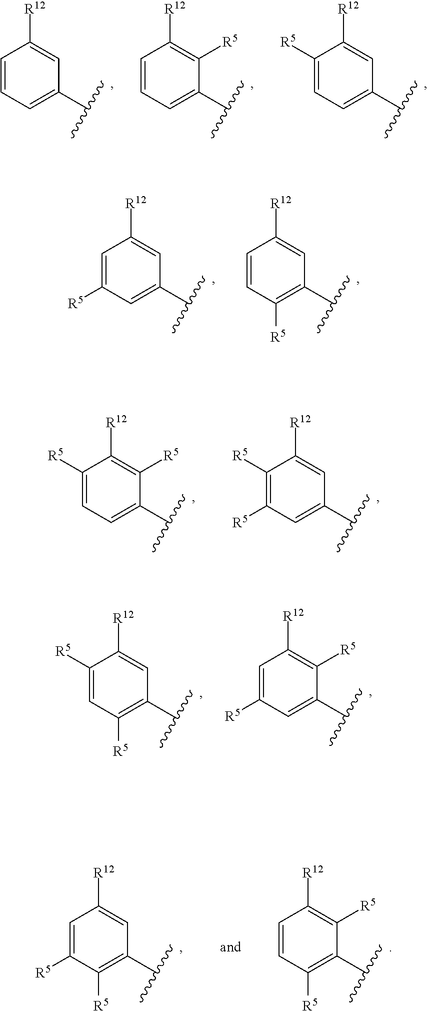

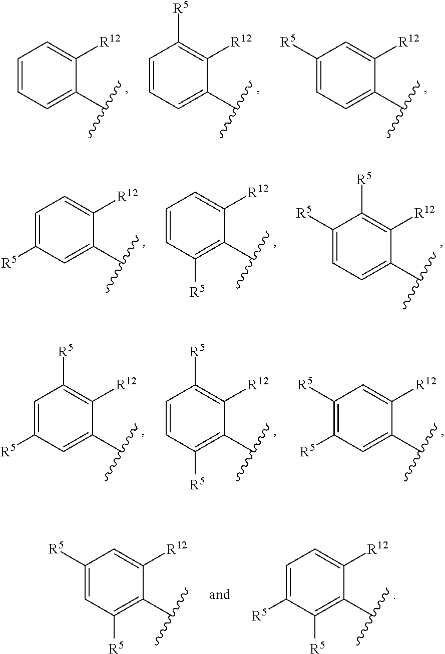

RR R5 R R⁵ and R5 A20. The compound of any one of embodiments A1-A13, wherein R¹⁶ and R¹⁷ are selected from the group consisting of:

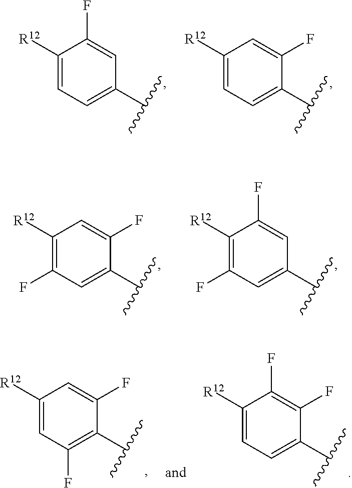

A21. The compound of embodiment A13 or embodiment A14, wherein R^{7A} and R¹ A are selected from the group consisting of:

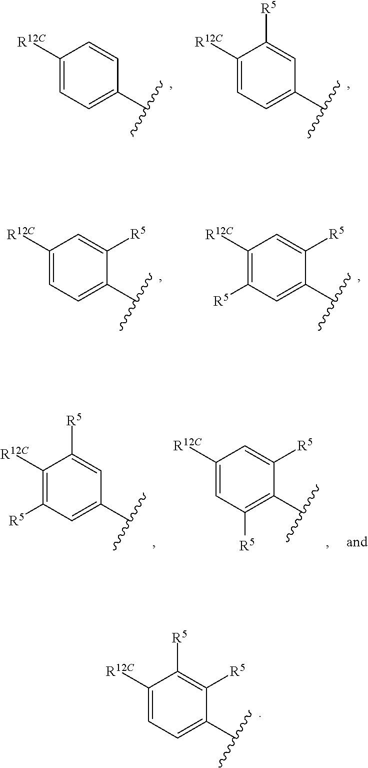

A22. The compound of any one of embodiments A1-A14, wherein R¹⁶, R¹⁷, R^{17A} and

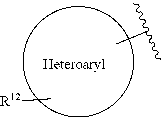

R1^{8A} are optionally substituted with 1 or 2 substituents independently selected from R⁵.

A23. The compound of any one of embodiments A1-A14, wherein R¹⁶, R¹⁷ R^{17A} and R1^{8A} are selected from the group consisting of: R5 R12 R12 R5 R^{12 hu l} R1 R12 5 R¹²N R⁵ R⁵ R R⁵

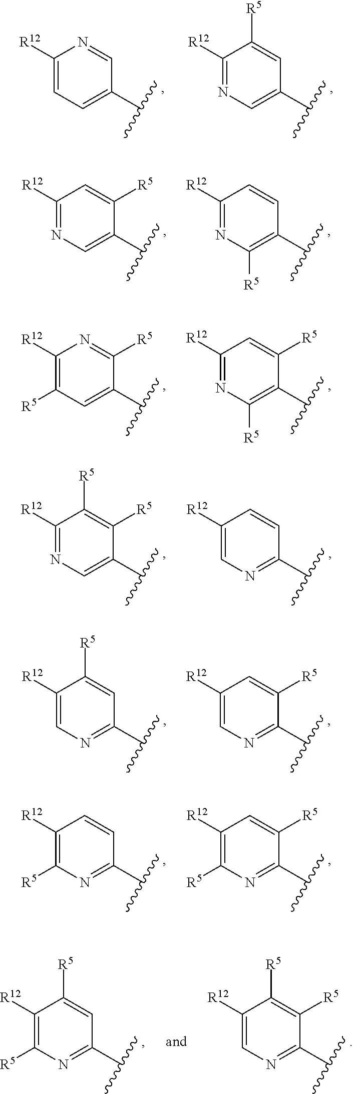

A24. The compound of any one of embodiments A1-A14, wherein R¹⁶, R¹⁷, R^{17A} and R1^{8A} are selected from the group consisting of: NN R R12 R and R¹² .

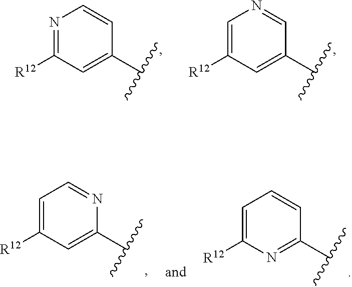

A25. The compound of any one of embodiments A1-A14, wherein R¹⁶, R¹⁷, R^{17A} and R1^{8A} are selected from the group consisting of: R5 R⁵ R5 R⁵ | N | N R5- |N N N N N N &O N. O N R12 R¹² R² R1² R² R12 R12 R2

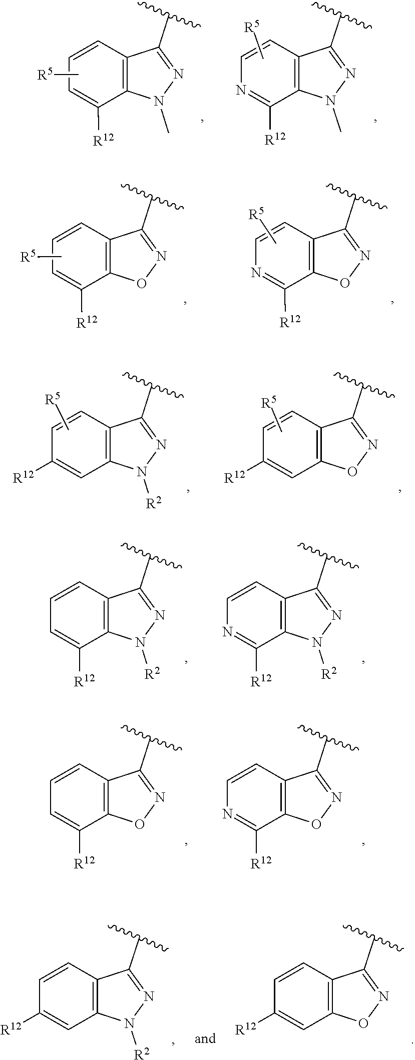

R5

N | N I N | N N A26. The compound of any one of embodiments A1-A14, wherein R¹⁶, R¹⁷ R1^{7A} and R^{18A} are selected from the group consisting of:

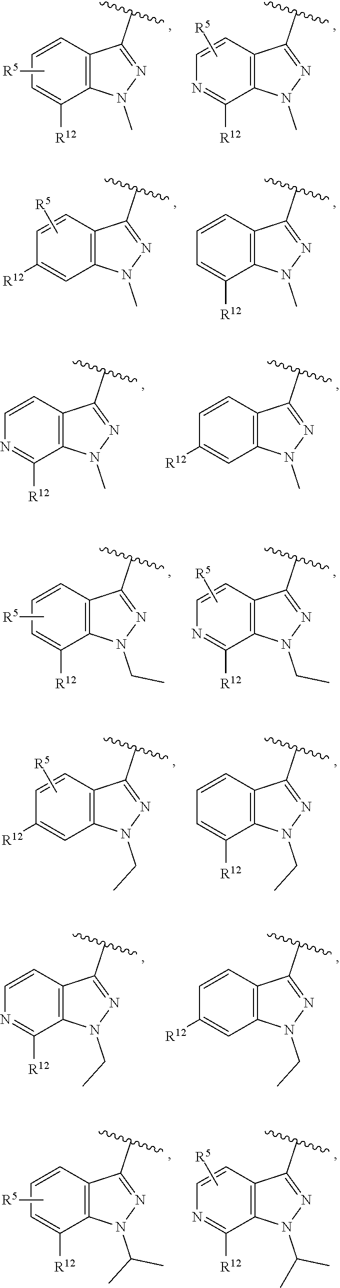

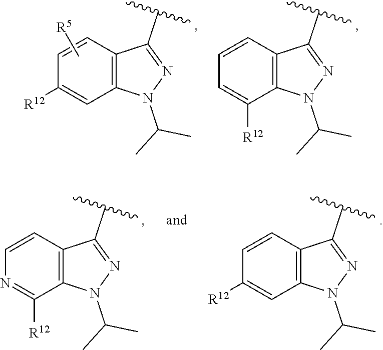

A27. The compound of any one of embodiments A1-A13, wherein R¹⁶, R¹⁷ and R^{17A} are selected from the group consisting of:

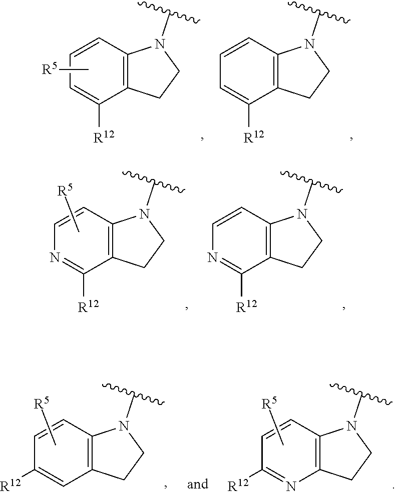

A28. The compound of any one of embodiments A1-A14, wherein R¹⁶, R⁷, R^{l7A} and

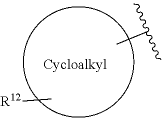

R¹sA are optionally substituted with 1 or 2 substituents independently selected from R⁵.

A29. The compound of any one of embodiments A1-A14, wherein R¹⁶, R¹⁷, R^{17A} and

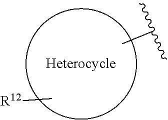

R¹sA are optionally substituted with 1 or 2 substituents independently selected from R⁵.

A30. The compound of any one of embodiments A1-A14, wherein R¹⁶, R¹⁷, R1^{7A} and

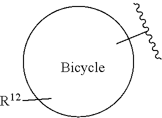

RIA are optionally substituted with 1 or 2 substituents independently selected from R⁵.

A31. The compound of embodiment A1 wherein the compound is of Formula:

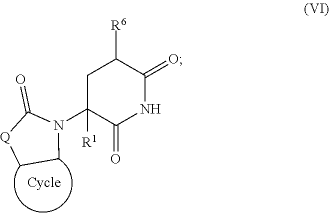

or a pharmaceutically acceptable salt thereof^{PGP}-5⁴⁰,C¹ A32. The compound of embodiment A31, wherein Q is NH.

A33. The compound of embodiment A31, wherein Q is NCH₃.

A34. The compound of embodiment A31, wherein Q is CH₂.

A35. The compound of embodiment A31, wherein Q is O.

A36. The compound of embodiment A31, wherein Q is S.

A37. The compound of any one of embodiments A31-A36, wherein Cycle is phenyl optionally substituted with 1 or 2 substituents independently selected from R⁵ and substituted with one R¹² substituent.

A38. The compound of embodiment A1 wherein the compound is of Formula:

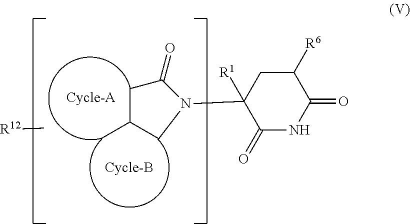

or a pharmaceutically acceptable salt thereof. A39. The compound of embodiment A38, wherein Cycle-A is phenyl optionally substituted with 1 or 2 substituents independently selected from Rsa A40. The compound of embodiment A38 or embodiment A39, wherein Cycle-B is phenyl optionally substituted with 1 or 2 substituents independently selected from R^{5b}.

A41. The compound of embodiment A1 wherein the compound is of Formula:

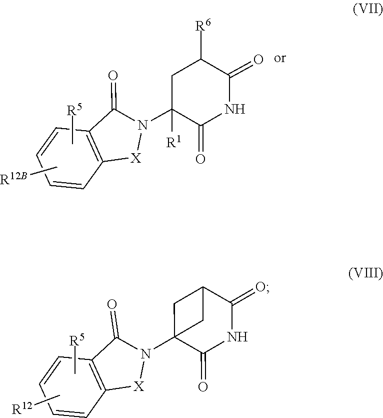

or a pharmaceutically acceptable salt thereof.

A42. The compound of embodiment A41, wherein X is CH₂.

A43. The compound of embodiment A41, wherein X is C(O). 0 0 SF A44. The compound of any one of embodiments A1-A43, wherein R¹² is 0F^{PGP}-5⁴³,C¹

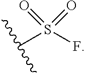 [SMILES: *S(=O)(=O)F]

A45. The compound of any one of embodiments A1-A43, wherein R¹² is fOF

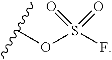 [SMILES: *OS(=O)(=O)F]

A46. The compound of any one of embodiments A1-A43, wherein R¹² is^{PGP}-5⁴⁴,C¹

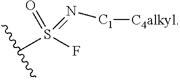

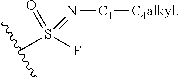

A48. The compound of any one of embodiments A41-A43, wherein R1^{2B} is

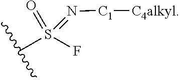

A49. The compound of any one of embodiments A41-A43, wherein R1^{2B} is

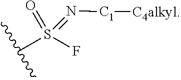

O,N-C₁-C₄ alkyl 0$0 A50. The compound of embodiment A16, wherein R^{12c} is 0 F 0.1

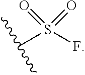 [SMILES: *S(=O)(=O)F]

0,N-C₁-C₄ alkyl A51. The compound of embodiment A16, wherein R^{12c} is F

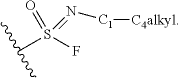

N SN-C₁-C₄ alkyl A52. The compound of embodiment A16, wherein R²c is 0 F

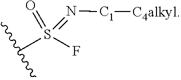

A53. The compound of any one of embodiments A1-A52, wherein R⁶ is hydrogen. ^{PGP}-5⁵¹,C¹ A54. The compound of any one of embodiments A1-A52, wherein R⁶ is CH₃.

A55. The compound of any one of embodiments A1-A54, wherein each R⁵ is independently selected from hydrogen, alkyl, haloalkyl, and halogen.

A56. The compound of any one of embodiments A1-A54, wherein each R⁵ is hydrogen.

A57. The compound of any one of embodiments A1-A54, wherein one R⁵ is F.

A58. The compound of any one of embodiments A1-A54, wherein one R⁵ is methyl.

A59. The compound of any one of embodiments A1-A54, wherein R⁵ is —NR⁷R⁸ or —OR⁷.

A60. The compound of embodiment A59, wherein R⁷ is hydrogen.

A61. The compound of embodiment A59, wherein R⁷ is methyl.

A62. The compound of any one of embodiments A59-A61, wherein R is hydrogen.

A63. The compound of any one of embodiments A59-A61, wherein R is methyl.

A64. The compound of any one of embodiments A1-A54, wherein R⁵ is aryl or heteroaryl optionally substituted with 1 or 2 substituents independently selected from RiOa A65. The compound of any one of embodiments A1-A54, wherein R⁵ is cyano.

A66. The compound of any one of embodiments A1-A54, wherein R⁵ is nitro.

A67. The compound of any one of embodiments A1-A54, wherein R⁵ -C(O)CH₃.

A68. The compound of any one of embodiments A1-A13 and A15-A67, wherein R¹ is hydrogen.

A69. The compound of any one of embodiments A1-A13 and A15-A67, wherein R¹ is CH₃.

A70. The compound of any one of embodiments A1-A13 and A15-A67, wherein R¹ and R⁶ combined to form a one-carbon bridge.

A71. A compound of Formula:

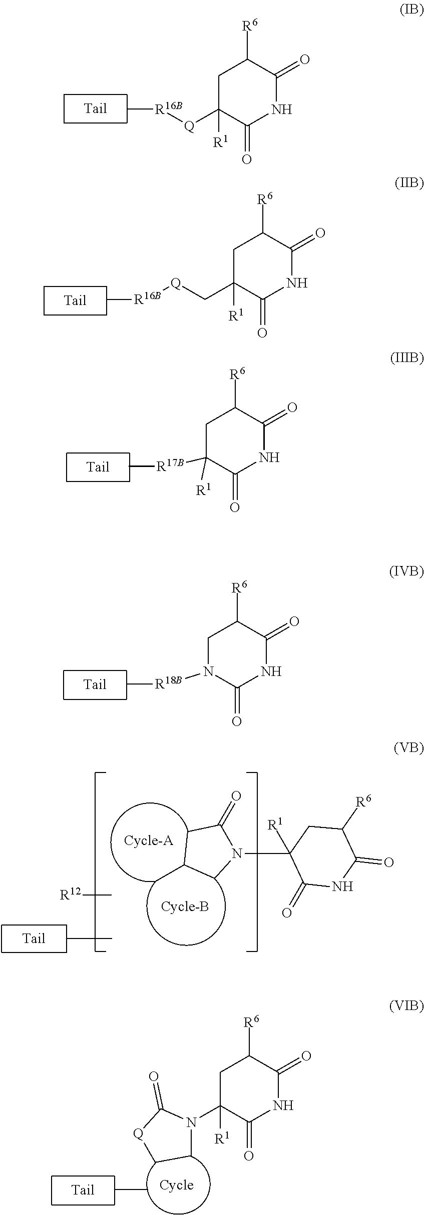

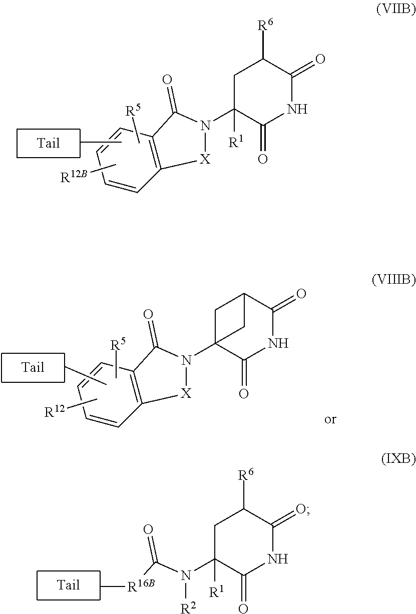

or a pharmaceutically acceptable salt thereof, wherein: Q is CH₂, NR2, O, or S; X is CH₂ or C(O); R¹ and R⁶ are independently selected from hydrogen, alkyl, alkenyl, alkynyl, and 10 halogen;
or R¹ and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from Rioa; 312R R 21 R2 Tail is of formula X³¹ R23 R21 X₂₂;

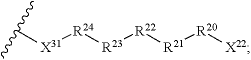

X³¹ is selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂- and —S—; each of which heterocycle, aryl, heteroaryl, and bicycle is substituted with 1, 2, or 3 substituents independently selected from R4°, X²² is selected from alkyl, haloalkyl, alkenyl, alkynyl, halogen, heteroaryl, heterocycle, —NR⁷R⁸, —OR⁷, —SR⁷, —C(O)R⁹, —C(S)R⁹, —S(O)R⁹, —S(O)₂R⁹, —OC(O)R⁹, —OC(S)R⁹, —OS(O)R⁹, —OS(O)₂R⁹, —SC(O)R⁹, —OS(O)₂R⁹, —NR⁷C(O)R⁹, —NR⁷C(S)R⁹, —NR⁷S(O)R⁹, —NR⁷S(O)₂R⁹, —P(O)(R⁹)₂, —SP(O)(R⁹)₂, —NR⁷P(O)(R⁹)₂, and —OP(O)(R⁹)₂; R²⁰, R²¹, R²², R²³, and R²⁴ are independently at each occurrence selected from the group 15 consisting of a bond, alkyl, —C(O)—, —C(O)O—, —OC(O)—, —SO2-, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkenyl, alkynyl, haloalkyl, alkoxy, aryl, heterocycle, heteroaryl, and carbocycle; each of which is optionally substituted with 1, 2, or 3 substituents independently selected from R⁴⁰; R²⁶ is independently at each occurrence selected from the group consisting of hydrogen, 20 alkyl, arylalkyl, heteroarylalkyl, alkenyl, alkynyl, aryl, heteroaryl, and heterocycle; R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkenyl, and alkynyl; R⁴⁰ is independently at each occurrence selected from the group consisting of hydrogen, 25 alkyl, alkenyl, alkynyl, fluoro, bromo, chloro, azide, OR⁷, NR⁷R⁸, S(O)NR⁷R⁸, S(O)2NR⁷R⁸, S(O)OR⁷, S(O)₂OR⁷, haloalkyl, aryl, heteroaryl, heterocycle, and cycloalkyl; R⁴¹ is independently at each occurrence selected from the group consisting of hydrogen, alkyl, alkenyl, alkynyl, haloalkyl, aryl, heteroaryl, heterocycle, and cycloalkyl; R″ ^{6B} is selected from:

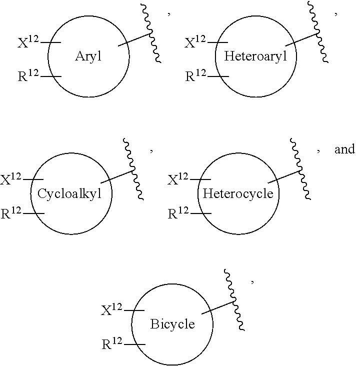

and each of which is optionally substituted with 1 or 2 substituents independently selected from R⁵; R¹⁷B is selected from:

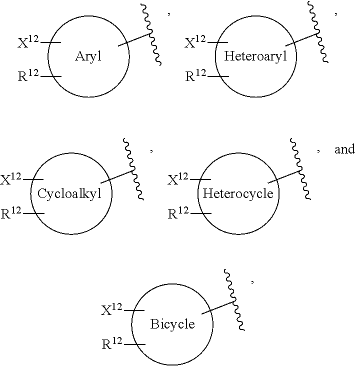

### X12 X12 X12 X12 Aryl Heteroaryl Cycloalkyl Heterocycle R12 R12 RR12 R12

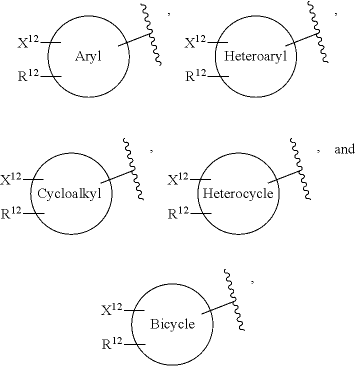

and, each of which is attached to the azaglutarimide moiety through a C—N bond and each of which R¹⁸B is optionally substituted with 1 or 2 substituents independently selected from R⁵; X¹² is the attachment point to Tail; Cycle is a fused aryl or heteroaryl group optionally substituted with 1 or 2 substituents independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a}.

Cycle-B is a fused ring selected from phenyl, 5- or 6-membered heteroaryl, 5- to 8-membered heterocycle, 5- to 8-membered cycloalkyl, or 5- to 8-membered cycloalkenyl, 5 wherein Cycle-B is optionally substituted with 1 or 2 substituents independently selected from R^{5b} O0 0 0 0,N-C₁,-C₄ alkyl O N-C₁-C₄ alkyl R¹² is selected from F 0 F F,and O‘ ’F

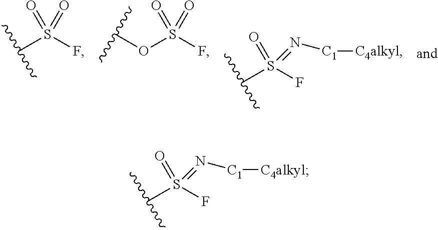

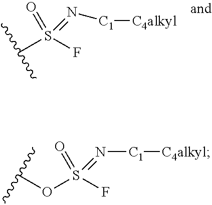

R⁷ and R at each instance are independently selected from hydrogen, alkyl, haloalkyl, 10 alkenyl, alkynyl, aryl, heteroaryl, heterocycle, and C(O)R¹⁴ each of which except hydrogen is optionally substituted with 1 or 2 substituents independently selected from R^{10b}each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R^{10a}, and R^{10b} is independently selected from hydrogen, alkyl, haloalkyl, 15 alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 20 2 substituents independently selected from R¹⁵;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; and each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and 25 —N(alkyl)₂.

A72. The compound of embodiment A71 wherein the compound is of Formula:

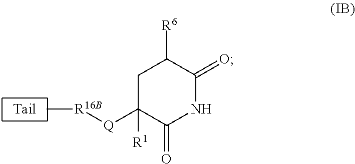

or a pharmaceutically acceptable salt thereof.

A73. The compound of embodiment A71 wherein the compound is of Formula:

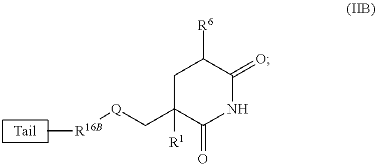

or a pharmaceutically acceptable salt thereof.

A74. The compound of any one of embodiments A71-A73, wherein Q is NH.

A75. The compound of any one of embodiments A71-A73, wherein Q is NCH₃.

A76. The compound of any one of embodiments A71-A73, wherein Q is CH₂.

A77. The compound of any one of embodiments A71-A73, wherein Q is O.

A78. The compound of any one of embodiments A71-A73, wherein Q is S.

A79. The compound of embodiment A71 wherein the compound is of Formula: R⁶ NH Tail R16B R R2 O (IXB);

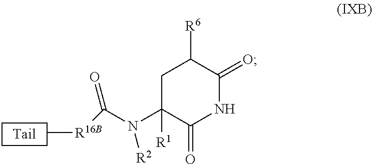

or a pharmaceutically acceptable salt thereof.

A80. The compound of embodiment A79 wherein R² is hydrogen.

A81. The compound of embodiment A79, wherein R² is methyl.

A82. The compound of embodiment A79, wherein R² is alkyl optionally substituted with 1 or 2 substituents independently selected from R¹⁰.

A83. The compound of embodiment A71 wherein the compound is of Formula:

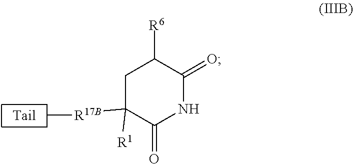

or a pharmaceutically acceptable salt thereof. A84. The compound of embodiment A71 wherein the compound is of Formula:

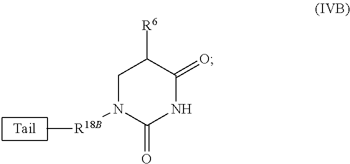

or a pharmaceutically acceptable salt thereof.

A85. The compound of any one of embodiments A71-A84, wherein R^{16B}, R^{17B} and R^{18B} are

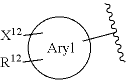

optionally substituted with 1 or 2 substituents independently selected from R⁵.

A86. The compound of any one of embodiments A71-A84, wherein R^{16B}, R^{17B} and

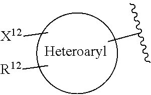

R^{18B} are optionally substituted with 1 or 2 substituents independently selected from R⁵.

A87. The compound of any one of embodiments A71-A84, wherein R^{16B}, R^{17B} and

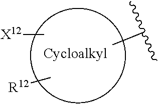

R^{18B} are R12 optionally substituted with 1 or 2 substituents independently selected from R⁵.

A88. The compound of any one of embodiments A71-A84, wherein R^{16B}, R^{17B} and X12 Heterocycle

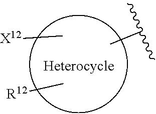

R^{18B} are R12 optionally substituted with 1 or 2 substituents independently selected from R⁵.

A89. The compound of any one of embodiments A71-A84, wherein R^{16B}, R^{17B} and

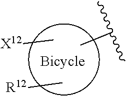

A90. The compound of embodiment A71 wherein the compound is of Formula:

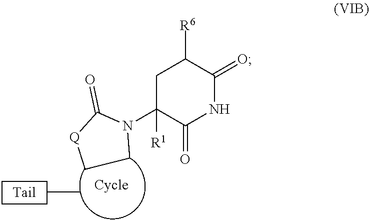

or a pharmaceutically acceptable salt thereof^{PGP}-5⁶⁹,C¹ A91. The compound of embodiment A90, wherein Q is NH.

A92. The compound of embodiment A90, wherein Q is NCH₃.

A93. The compound of embodiment A90, wherein Q is CH₂.

A94. The compound of embodiment A90, wherein Q is O.

A95. The compound of embodiment A90, wherein Q is S.

A96. The compound of any one of embodiments A90-A95, wherein Cycle is phenyl optionally substituted with 1 or 2 substituents independently selected from R⁵ and substituted with one R¹² substituent.

A97. The compound of embodiment A71, wherein the compound is of Formula:

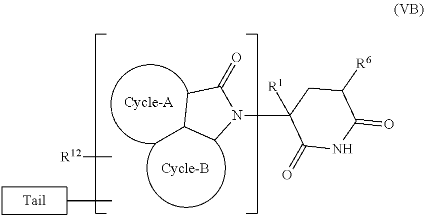

or a pharmaceutically acceptable salt thereof.

A98. The compound of embodiment A97, wherein Cycle-A is phenyl optionally substituted with 1 or 2 substituents independently selected from Rsa A99. The compound of embodiment A97 or embodiment 98, wherein Cycle-B is phenyl optionally substituted with 1 or 2 substituents independently selected from R^{5b}. A100. The compound of embodiment A71 wherein the compound is of Formula:

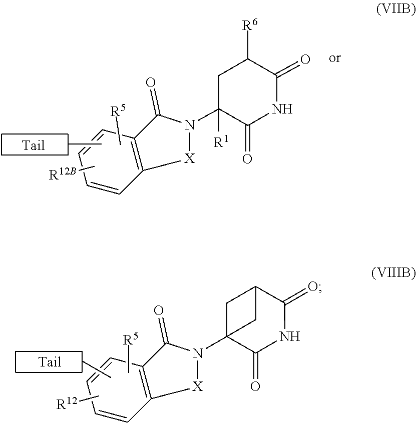

or a pharmaceutically acceptable salt thereof.

A101. The compound of embodiment A100, wherein X is CH₂.

A102. The compound of embodiment A100, wherein X is C(O).

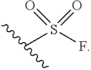 [SMILES: *S(=O)(=O)F]

A103. The compound of any one of embodiments A71-A102, wherein R¹² is F A104. The compound of any one of embodiments A71-A102, wherein R¹² is O 1

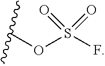 [SMILES: *OS(=O)(=O)F]

A10. The compound of any one of embodiments A71-A102, wherein R¹² is

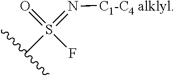

A106. The compound of any one of embodiments A71-A102, wherein R¹² is

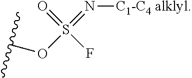

A107. The compound of any one of embodiments A100-A102, wherein R¹²B is

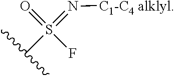

A108. The compound of any one of embodiments A100-A102, wherein R¹²B is

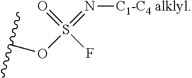

A109. The compound of any one of embodiments A71-A108, wherein R⁶ is hydrogen.

Al10. The compound of any one of embodiments A71-A108, wherein R⁶ is CH₃.

All1. The compound of any one of embodiments A71-A110, wherein each R⁵ is independently selected from hydrogen, alkyl, haloalkyl, and halogen.

A1 12. The compound of any one of embodiments A71-A110, wherein each R⁵ is hydrogen.

A113. The compound of any one of embodiments A71-A110, wherein one R⁵ is F.

A1 14. The compound of any one of embodiments A71-A110, wherein one R⁵ is methyl.

A1 15. The compound of any one of embodiments A71-A110, wherein R⁵ is —NR⁷R or —OR⁷.

A1 16. The compound of embodiment A1 15, wherein R⁷ is hydrogen.

A1 17. The compound of embodiment A1 15, wherein R⁷ is methyl.

A1 18. The compound of any one of embodiments Al15-Al17, wherein R is hydrogen.

A119. The compound of any one of embodiments A115-A117, wherein R is methyl.

A120. The compound of any one of embodiments A71-A110, wherein R⁵ is aryl or heteroaryl optionally substituted with 1, 2, or 3 substituents independently selected from R¹⁰ .

A121. The compound of any one of embodiments A71-A110, wherein R⁵ is cyano.

A122. The compound of any one of embodiments A71-A110, wherein R⁵ is nitro.

A123. The compound of any one of embodiments A71-A110, wherein R⁵ -C(O)CH₃.

A124. The compound of any one of embodiments A71-A83 and A85-A123, wherein R¹ is hydrogen.

A125. The compound of any one of embodiments A71-A83 and A85-A123, wherein R¹ is CH₃.

A126. The compound of any one of embodiments A71-A83 and A85-A123, wherein R¹ and R⁶ combined form a one-carbon bridge.

A127. The compound of any one of embodiments A71-A126, wherein Tail is of formula:

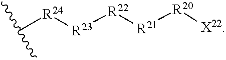

A128. The compound of any one ofembodiments A71-A127, wherein R²⁰ is bond.

A129. The compound of any one of embodiments A71-A127, wherein R²⁰ is CH₂. A130. The compound of any one of embodiments A71-A127, wherein R²⁰ is heterocycle optionally substituted with 1, 2, or 3 substituents independently selected from R⁴⁰ .

A131. The compound of any one of embodiments A71-A127, wherein R²⁰ is aryl optionally substituted with 1, 2, or 3 substituents independently selected from R⁴⁰.

A132. The compound of any one of embodiments A71-A127, wherein R²⁰ is phenyl optionally substituted with 1, 2, or 3 substituents independently selected from R⁴⁰.

A133. The compound of any one of embodiments A71-A127, wherein R²⁰ is bicycle optionally substituted with 1, 2, or 3 substituents independently selected from R⁴⁰.

A134. The compound of any one of embodiments A71-A133, wherein R²¹ is bond.

A135. The compound of any one of embodiments A71-A133, wherein R²¹ is CH₂.

A136. The compound of any one of embodiments A71-A133, wherein R²¹ is heterocycle optionally substituted with 1, 2, or 3 substituents independently selected from R⁴⁰ .

A137. The compound of any one of embodiments A71-A133, wherein R²¹ is aryl optionally substituted with 1, 2, or 3 substituents independently selected from R⁴⁰.

A138. The compound of any one of embodiments A71-A133, wherein R²¹ is phenyl optionally substituted with 1, 2, or 3 substituents independently selected from R⁴⁰.

A139. The compound of any one of embodiments A71-A133, wherein R²¹ is bicycle optionally substituted with 1, 2, or 3 substituents independently selected from R⁴⁰.

A140. The compound of any one of embodiments A71-A139, wherein R²² is bond.

A141. The compound of any one of embodiments A71-A139, wherein R²² is CH₂.

A142. The compound of any one of embodiments A71-A139, wherein R²² is heterocycle optionally substituted with 1, 2, or 3 substituents independently selected from R⁴⁰ .

A143. The compound of any one of embodiments A71-A139, wherein R²² is aryl optionally substituted with 1, 2, or 3 substituents independently selected from R⁴⁰.

A144. The compound of any one of embodiments A71-A139, wherein R²² is phenyl optionally substituted with 1, 2, or 3 substituents independently selected from R⁴⁰.

A145. The compound of any one of embodiments A71-A139, wherein R²² is bicycle optionally substituted with 1, 2, or 3 substituents independently selected from R⁴⁰.

A146. The compound of any one of embodiments A71-A145, wherein R²³ is bond.

A147. The compound of any one of embodiments A71-A145, wherein R²³ is CH₂.

A148. The compound of any one of embodiments A71-A145, wherein R²³ is heterocycle optionally substituted with 1, 2, or 3 substituents independently selected from R⁴⁰ .

A149. The compound of any one of embodiments A71-A145, wherein R²³ is aryl optionally substituted with 1, 2, or 3 substituents independently selected from R⁴⁰ .

A150. The compound of any one of embodiments A71-A145, wherein R²³ is phenyl optionally substituted with 1, 2, or 3 substituents independently selected from R⁴⁰ .

A151. The compound of any one of embodiments A71-A145, wherein R²³ is bicycle optionally substituted with 1, 2, or 3 substituents independently selected from R⁴⁰ .

A152. The compound of any one of embodiments A71-A151, wherein R²⁴ is bond.

A153. The compound of any one of embodiments A71-A151, wherein R²⁴ is CH₂.

A154. The compound of any one of embodiments A71-A151, wherein R²⁴ is heterocycle optionally substituted with 1, 2, or 3 substituents independently selected from R⁴⁰ .

A155. The compound of any one of embodiments A71-A151, wherein R²⁴ is aryl optionally substituted with 1, 2, or 3 substituents independently selected from R⁴⁰.

A156. The compound of any one of embodiments A71-A151, wherein R²⁴ is phenyl optionally substituted with 1, 2, or 3 substituents independently selected from R⁴⁰.

A157. The compound of any one of embodiments A71-A151, wherein R²⁴ is bicycle optionally substituted with 1, 2, or 3 substituents independently selected from R⁴⁰.

A158. The compound of any one of embodiments A71-A151, wherein R²⁴ is C(O).

A159. The compound of any one of embodiments A71-A126 wherein Tail is selected from

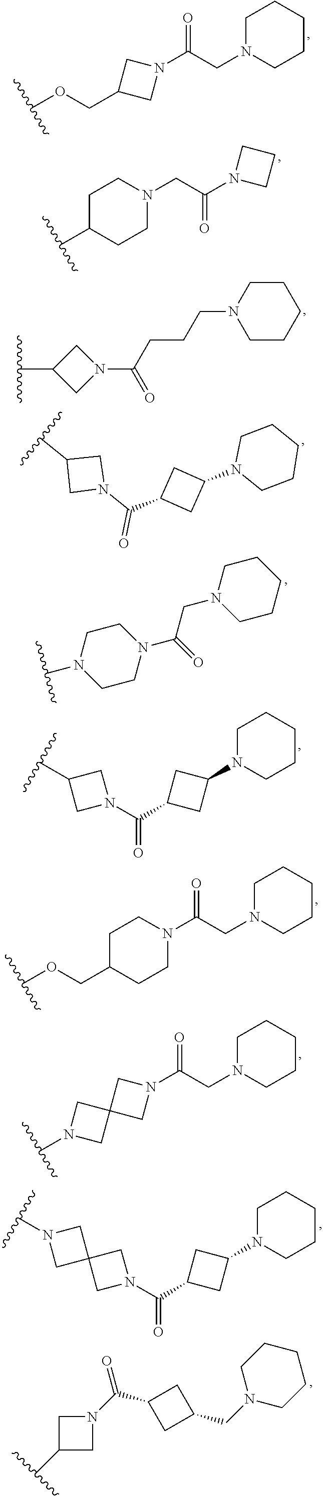

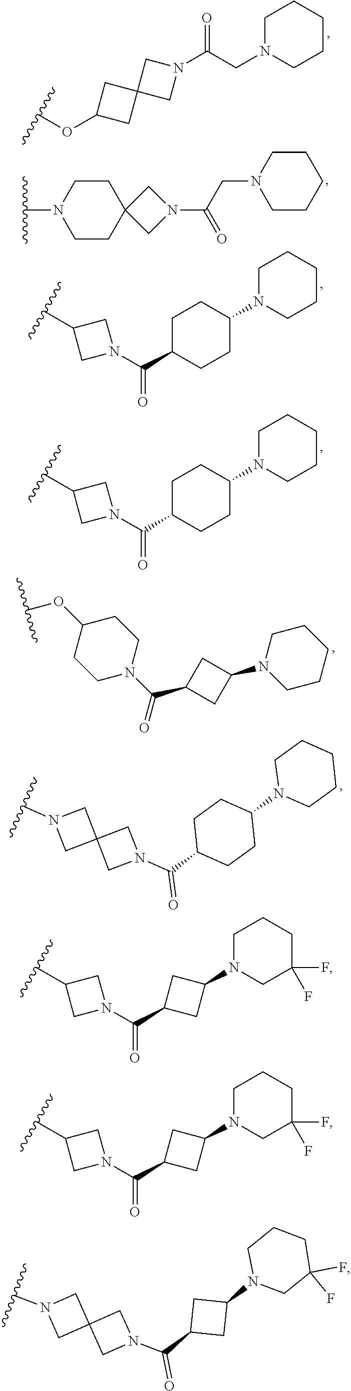

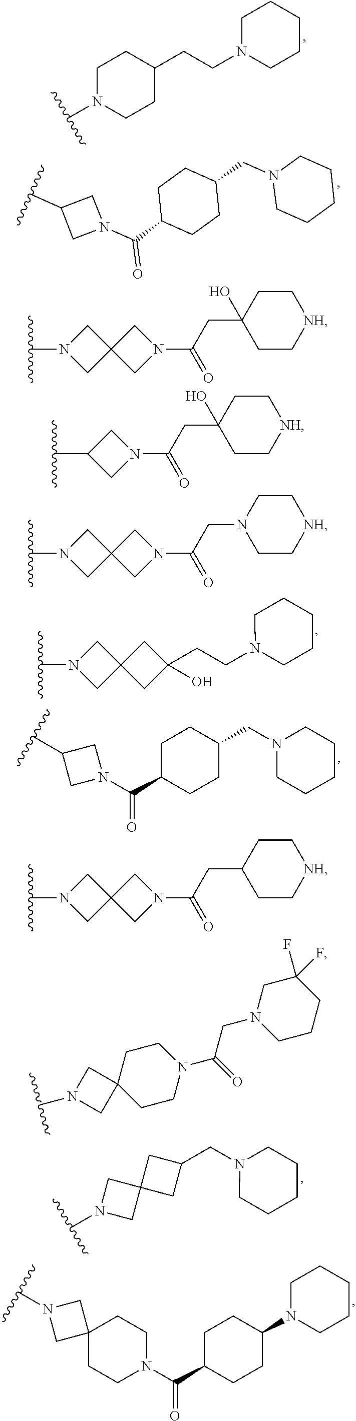

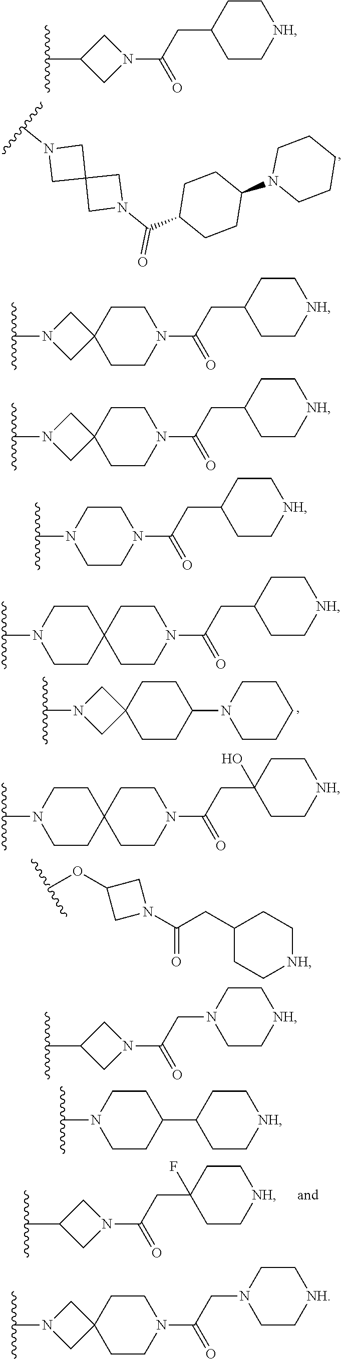

A160. The compound of any one of embodiments A71-A126 wherein Tail is selected from

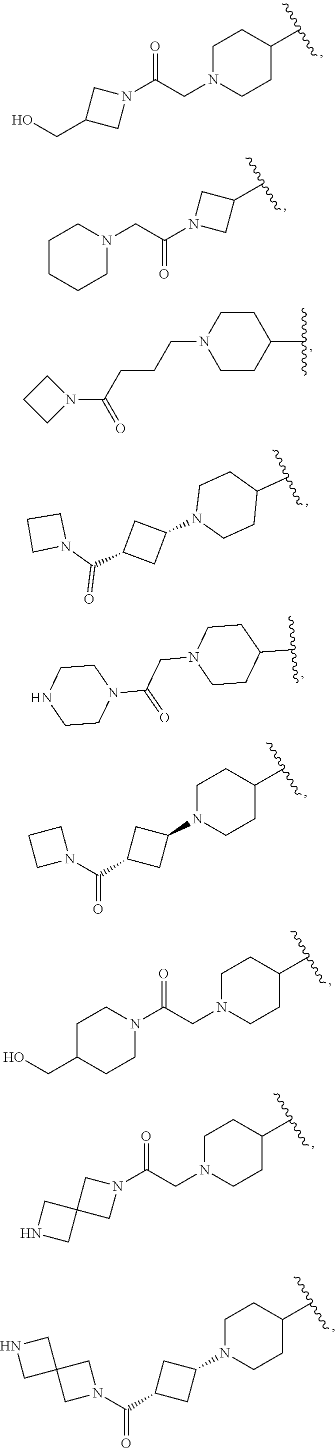

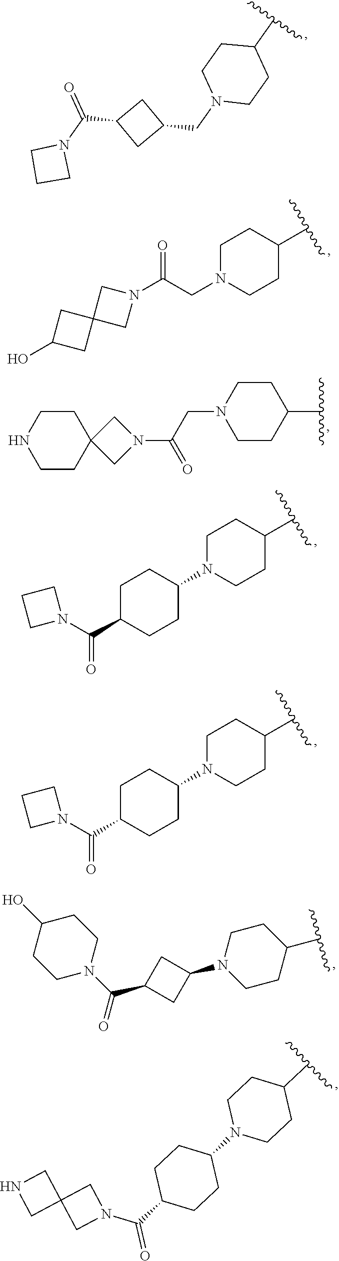

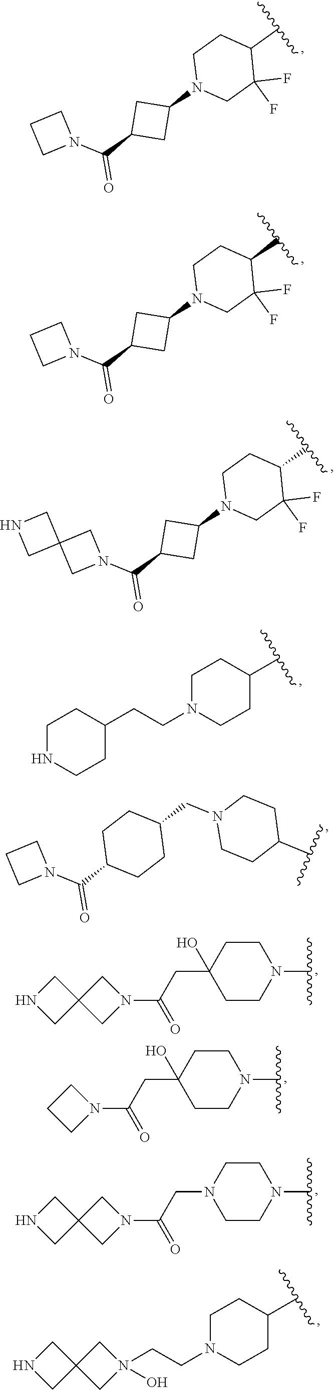

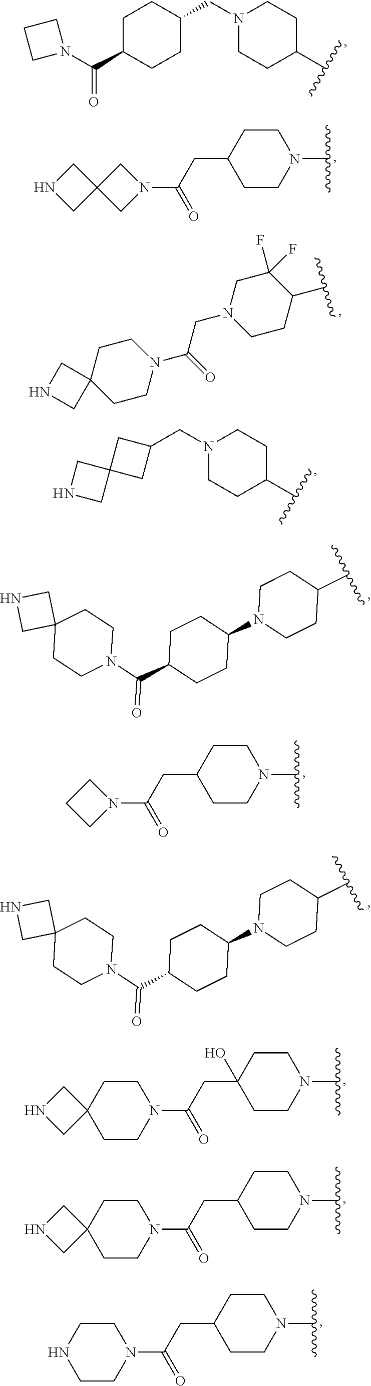

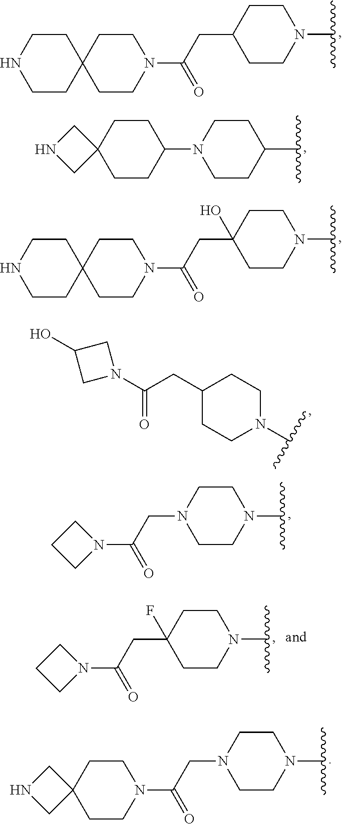

A161. A pharmaceutical composition comprising a compound of any one of embodiments A1-A160 or a pharmaceutically acceptable salt thereof and a pharmaceutically acceptable carrier.

A162. A method of treating a disorder comprising administering an effective amount of a compound of any one of embodiments A1-A160 or a pharmaceutical composition of embodiment A161, wherein the disorder is mediated by a protein that is degraded or downregulated by the compound.

A163. The method of embodiment A162, wherein the protein is a zinc finger protein.

A164. The method of embodiment A162, wherein the protein is selected from: ARID2, CDK1, CDK12, CDK13, CK1alpha, CSNK1A1, Cyclin K, E4F1, FAM83F, GSPT1, GSPT2, GZF1, IKZF1, IKZF2, IKZF3, IKZF4, ILF2, Myc, ODC1, p63, PDE6D, AB28, RARalpha-ZBTB16, RBM23, RBM39, RBM39, RNF166, SALL4, WBP4, ZBTB16, ZBTB16-RARalpha, ZBTB39, ZFP91, ZFP91, ZFP91, ZMYM2-FGFR1, ZMYM2-FLT3, ZNF198, ZNF276, ZNF276, ZNF517, ZNF582, ZNF653, ZNF654, ZNF692, ZNF787, ZNF827, and ZNF98.

A165. The method of embodiment A162, wherein the protein is selected from: ARID2, aromatase, CDK12, NRF2, PDE6D, CK1alpha, cyclin K, GSPT1, FAM83, ILF2, ZBTB16, and ZMYM2.

A166. A compound of Formula:

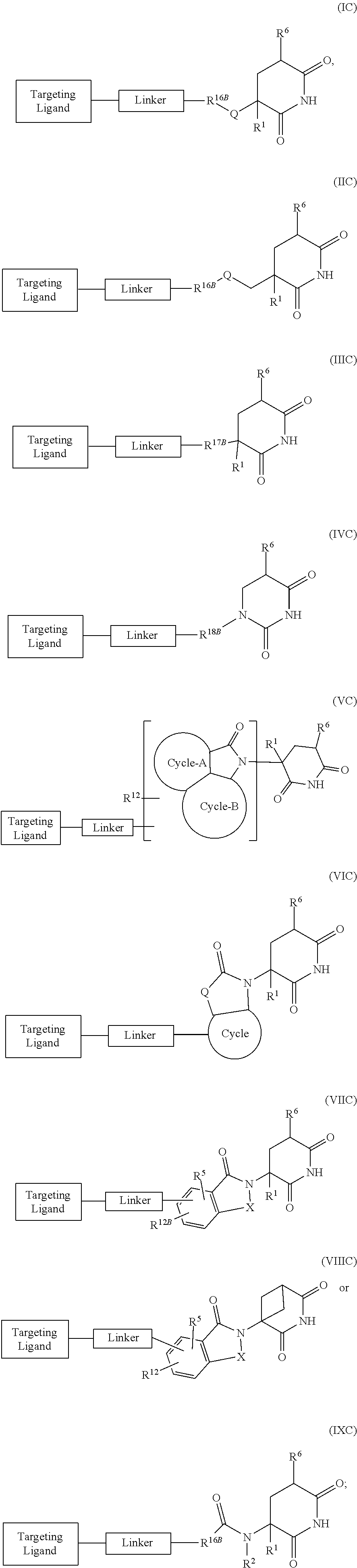

or a pharmaceutically acceptable salt thereof, wherein: 5 Q is CH₂, NR², O, or S; X is CH₂ or C(O); R¹ and R⁶ are independently selected from hydrogen, alkyl, alkenyl, alkynyl, and halogen;
or R¹ and R⁶ are combined to form a one or two carbon bridge to form a fused cycle; each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, 15 -C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a}; Linker is of Formula:

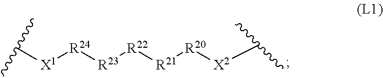

X¹ and X² are independently at each occurrence selected from bond, carbamate, heterocycle, NR⁴², C(R⁴²)2, O, C(O), and S; R⁴² is independently at each occurrence selected from the group consisting of hydrogen, alkyl, heteroalkyl, heterocycle, aryl, heteroaryl, —C(O)H, —C(O)OH, —C(O)alkyl, —C(O)Oalkyl, —C(O)(aryl, heteroalkyl or heteroaryl), —C(O)O(aryl, heteroalkyl, or heteroaryl), alkenyl, and alkynyl; R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkenyl, alkynyl, haloalkyl, alkoxy, aryl, heterocycle, heteroaryl, and carbocycle; each of which is optionally substituted with 1, 2, or 3 substituents independently selected from R⁴⁰; R²⁶ is independently at each occurrence selected from the group consisting of hydrogen, alkyl, arylalkyl, heteroarylalkyl, alkenyl, alkynyl, aryl, heteroaryl, and heterocycle; R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkenyl, and alkynyl; R⁴⁰ is independently at each occurrence selected from the group consisting of hydrogen, alkyl, alkenyl, alkynyl, fluoro, bromo, chloro, azide, OR⁷, NR⁷R⁸, S(O)NR⁷R⁸, S(O)2NR⁷R8, S(O)OR⁷, S(O)₂OR⁷, haloalkyl, aryl, heteroaryl, heterocycle, and cycloalkyl; R⁴¹ is independently at each occurrence selected from the group consisting of hydrogen, alkyl, alkenyl, alkynyl, haloalkyl, aryl, heteroaryl, heterocycle, and cycloalkyl; R¹⁶B is selected from:

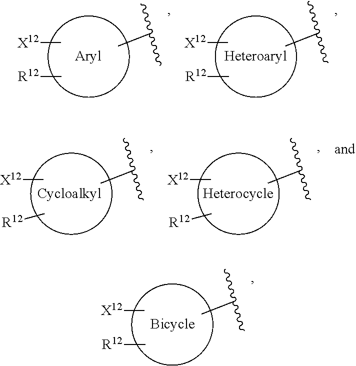

and each of which is optionally substituted with 1 or 2 substituents independently selected from R⁵; R¹⁷B is selected from:

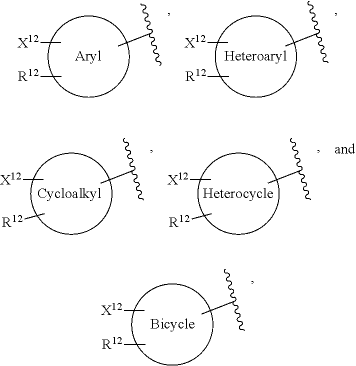

### X12 X12 X12 1 Aryl Heteroaryl Cycloalkyl Heterocycle R12 R12 R1z R1z X12 Bicycle

and, each of which is optionally substituted with 1 or 2 substituents independently selected from R⁵; R″ ” is selected from:

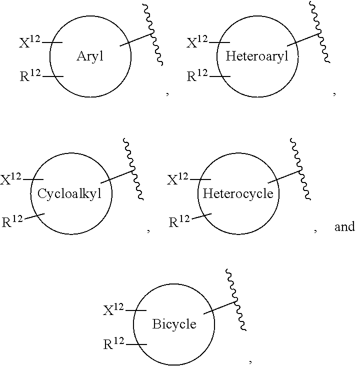

and, each of which is attached to the azaglutarimide moiety through a C—N bond and each of which Ri^{8B} is optionally substituted with 1 or 2 substituents 5 independently selected from R⁵; X¹² is the attachment point to Linker; Cycle is a fused aryl or heteroaryl group optionally substituted with 1 or 2 substituents independently selected from R⁵ and substituted with one R¹² substituent; Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a}.

Cycle-B is a fused ring selected from phenyl, 5- or 6-membered heteroaryl, 5- to 8-membered heterocycle, 5- to 8-membered cycloalkyl, or 5- to 8-membered cycloalkenyl, 15 wherein Cycle-B is optionally substituted with 1 or 2 substituents independently selected from R^{5b}

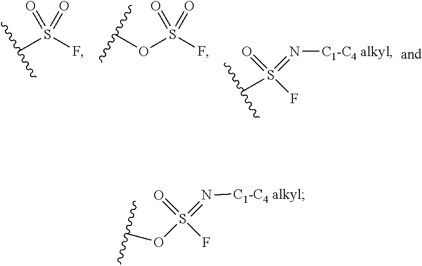

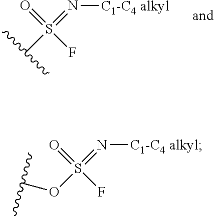

R¹²B is selected from F and 0 F R⁷ and R at each instance are independently selected from hydrogen, alkyl, haloalkyl, 20 alkenyl, alkynyl, aryl, heteroaryl, heterocycle, and C(O)R¹⁴ each of which except hydrogen is optionally substituted with 1 or 2 substituents independently selected from R^{10b}each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R^{10a}, and R^{10b} is independently selected from hydrogen, alkyl, haloalkyl, 25 alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂; R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R″;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)₂; each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂; and Targeting Ligand is a ligand that binds to a Target Protein.

A167. The compound of embodiment A166 wherein the compound is of Formula:

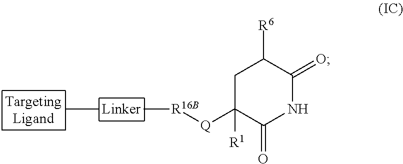

or a pharmaceutically acceptable salt thereof. A168. The compound of embodiment A166 wherein the compound is of Formula:

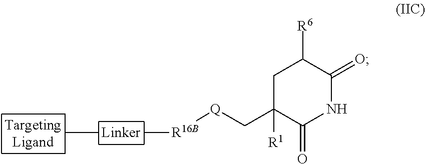

or a pharmaceutically acceptable salt thereof. A169. The compound of any one of embodiments A166-A168, wherein Q is NH.

A170. The compound of any one of embodiments A166-A168, wherein Q is NCH₃.

A171. The compound of any one of embodiments A166-A168, wherein Q is CH₂.

A172. The compound of any one of embodiments A166-A168, wherein Q is O.

A173. The compound of any one of embodiments A166-A168, wherein Q is S. A174. The compound of embodiment A166, wherein the compound is of Formula:

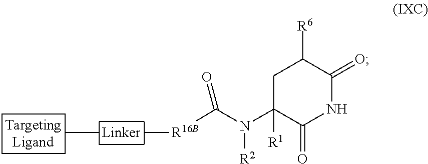

or a pharmaceutically acceptable salt thereof. A175. The compound of embodiment A174, wherein R² is hydrogen.

A176. The compound of embodiment A174, wherein R² is methyl.

A177. The compound of embodiment A174, wherein R² is alkyl optionally substituted with 1 or 2 substituents independently selected from R¹⁰.

A178. The compound of embodiment A166 wherein the compound is of Formula:

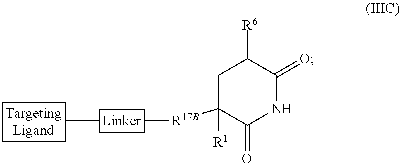

or a pharmaceutically acceptable salt thereof. A179. The compound of embodiment A166 wherein the compound is of Formula:

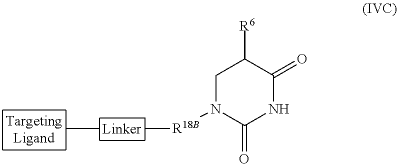

or a pharmaceutically acceptable salt thereof.

A180. The compound of any one of embodiments A166-A179, wherein R^{16B}, R^{17B} and

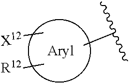

R^{18B} are optionally substituted with 1 or 2 substituents independently selected from R⁵.

A181. The compound of any one of embodiments A166-A179, wherein R¹⁶B, R^{17B} and

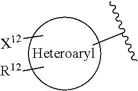

R^{18B} are optionally substituted with 1 or 2 substituents independently selected from R⁵.

A182. The compound of any one of embodiments A166-A179, wherein R^{16B}, R^{17B} and Cycloalkyl

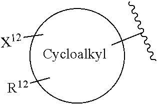

R^{18B} are R12 optionally substituted with 1 or 2 substituents independently selected from R⁵.

A183. The compound of any one of embodiments A166-A179, wherein R^{16B}, R^{17B} and

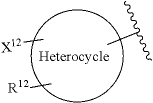

R^{18B} are R^{2 hu l} optionally substituted with 1 or 2 substituents independently selected from R⁵.

A184. The compound of any one of embodiments A166-A179, wherein R^{16B}, R^{17B} and

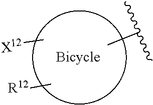

R^{18B} are optionally substituted with 1 or 2 substituents independently selected from R⁵.

A185. The compound of embodiment A166 wherein the compound is of Formula:

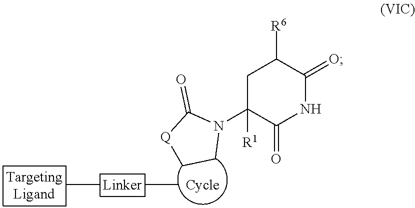

or a pharmaceutically acceptable salt thereof.

A186. The compound of embodiment A185, wherein Q is NH.

A187. The compound of embodiment A185, wherein Q is NCH₃.

A188. The compound of embodiment A185, wherein Q is CH₂.

A189. The compound of embodiment A185, wherein Q is O.

A190. The compound of embodiment A185, wherein Q is S.

A191. The compound of any one of embodiments A185-A190, wherein Cycle is phenyl optionally substituted with 1 or 2 substituents independently selected from R⁵ and substituted with one R¹² substituent.

A192. The compound of embodiment A166 wherein the compound is of Formula:

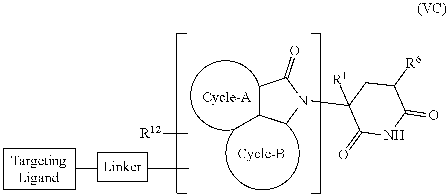

or a pharmaceutically acceptable salt thereof. A193. The compound of embodiment A192, wherein Cycle-A is phenyl optionally substituted with 1 or 2 substituents independently selected from Rs.

A194. The compound of embodiment A192 or embodiment A193, wherein Cycle-B is phenyl optionally substituted with 1 or 2 substituents independently selected from R^{5b}.

A195. The compound of embodiment A166 wherein the compound is of Formula:

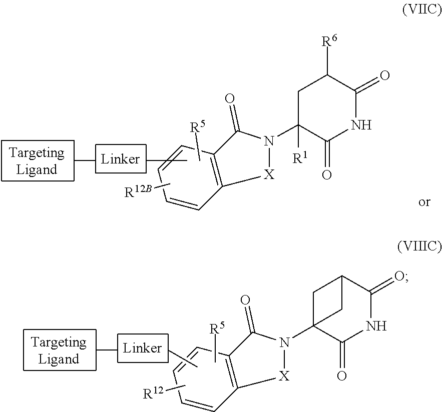

or a pharmaceutically acceptable salt thereof. A196. The compound of embodiment A195, wherein X is CH₂.

A197. The compound of embodiment A195, wherein X is C(O).

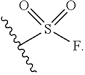 [SMILES: *S(=O)(=O)F]

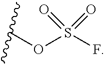 [SMILES: *OS(=O)(=O)F]

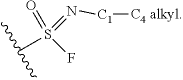

A201. The compound of any one of embodiments A166-A197, wherein R¹² is , N—C₁-C₄ alkyl 0 F.

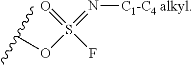

A202. The compound of any one of embodiments A195-A197, wherein R²B is S,N-C₁-C₄ alkyl SF

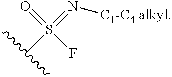

A203. The compound of any one of embodiments A195-A197, wherein R²B is

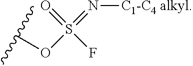

A204. The compound of any one of embodiments A166-A203, wherein R⁶ is hydrogen.

A205. The compound of any one of embodiments A166-A203, wherein R⁶ is CH₃.

A206. The compound of any one of embodiments A166-A205, wherein each R⁵ is independently selected from hydrogen, alkyl, haloalkyl, and halogen.

A207. The compound of any one of embodiments A166-A205, wherein each R⁵ is hydrogen.

A208. The compound of any one of embodiments A166-A205, wherein one R⁵ is F.

A209. The compound of any one of embodiments A166-A205, wherein one R⁵ is methyl.

A210. The compound of any one of embodiments A166-A205, wherein R⁵ is —NR⁷R or —OR⁷.

A211. The compound of embodiment A210, wherein R⁷ is hydrogen.

A212. The compound of embodiment A210, wherein R⁷ is methyl.

A213. The compound of any one of embodiments A210-A212, wherein R is hydrogen.

A214. The compound of any one of embodiments A210-A212, wherein R is methyl.

A215. The compound of any one of embodiments A166-A205, wherein R⁵ is aryl or heteroaryl optionally substituted with 1, 2, or 3 substituents independently selected from R¹⁰ .

A216. The compound of any one of embodiments A166-A205, wherein R⁵ is cyano.

A217. The compound of any one of embodiments A166-A205, wherein R⁵ is nitro.

A218. The compound of any one of embodiments A166-A205, wherein R⁵ -C(O)CH₃.

A219. The compound of any one of embodiments A166-A178 and A180-A218, wherein R¹ is hydrogen.

A220. The compound of any one of embodiments A166-A178 and A180-A218, wherein R¹ is CH₃.

A221. The compound of any one of embodiments A166-A178 and A180-A218, wherein R¹ and R⁶ combined form a one-carbon bridge.

A222. The compound of any one of embodiments A166-A221, wherein Linker is of formula:

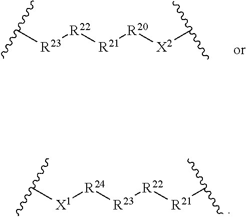

A223. The compound of any one of embodiments A166-A222, wherein X¹ is bond.

A224. The compound of any one of embodiments A166-A222, wherein X¹ is heterocycle.

A225. The compound of any one of embodiments A166-A222, wherein X¹ is NR².

A226. The compound of any one of embodiments A166-A222, wherein X¹ is C(O).

A227. The compound of any one of embodiments A166-A226, wherein X² is bond.

A228. The compound of any one of embodiments A166-A226, wherein X² is heterocycle.

A229. The compound of any one of embodiments A166-A226, wherein X² is NR².

A230. The compound of any one of embodiments A166-A226, wherein X² is C(O).

A231. The compound of any one of embodiments A166-A230, wherein R²⁰ is bond.

A232. The compound of any one of embodiments A166-A230, wherein R²⁰ is CH₂.

A233. The compound of any one of embodiments A166-A230, wherein R²⁰ is heterocycle optionally substituted with 1, 2, or 3 substituents independently selected from R⁴⁰ .

A234. The compound of any one of embodiments A166-A230, wherein R²⁰ is aryl optionally substituted with 1, 2, or 3 substituents independently selected from R⁴⁰. A235. The compound of any one of embodiments A166-A230, wherein R²⁰ is phenyl optionally substituted with 1, 2, or 3 substituents independently selected from R⁴⁰ .

A236. The compound of any one of embodiments A166-A230, wherein R²⁰ is bicycle optionally substituted with 1, 2, or 3 substituents independently selected from R⁴⁰ .

A237. The compound of any one of embodiments A166-A236, wherein R²¹ is bond.

A238. The compound of any one of embodiments A166-A236, wherein R²¹ is CH₂.

A239. The compound of any one of embodiments A166-A236, wherein R²¹ is heterocycle optionally substituted with 1, 2, or 3 substituents independently selected from R⁴⁰ .

A240. The compound of any one of embodiments A166-A236, wherein R²¹ is aryl optionally substituted with 1, 2, or 3 substituents independently selected from R⁴⁰.

A241. The compound of any one of embodiments A166-A236, wherein R²¹ is phenyl optionally substituted with 1, 2, or 3 substituents independently selected from R⁴⁰.

A242. The compound of any one of embodiments A166-A236, wherein R²¹ is bicycle optionally substituted with 1, 2, or 3 substituents independently selected from R⁴⁰.

A243. The compound of any one of embodiments A166-A221, wherein Linker is of formula:

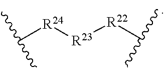

A244. The compound of any one of embodiments A166-A243, wherein R²² is bond.

A245. The compound of any one of embodiments A166-A243, wherein R²² is CH₂.

A246. The compound of any one of embodiments A166-A243, wherein R²² is heterocycle optionally substituted with 1, 2, or 3 substituents independently selected from R⁴⁰ .

A247. The compound of any one of embodiments A166-A243, wherein R²² is aryl optionally substituted with 1, 2, or 3 substituents independently selected from R⁴⁰.

A248. The compound of any one of embodiments A166-A243, wherein R²² is phenyl optionally substituted with 1, 2, or 3 substituents independently selected from R⁴⁰.

A249. The compound of any one of embodiments A166-A243, wherein R²² is bicycle optionally substituted with 1, 2, or 3 substituents independently selected from R⁴⁰.

A250. The compound of any one of embodiments A166-A221, wherein Linker is of formula:

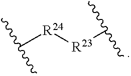

A251. The compound of any one of embodiments A166-A250, wherein R²³ is bond.

A252. The compound of any one of embodiments A166-A250, wherein R²³ is CH₂.

A253. The compound of any one of embodiments A166-A250, wherein R²³ is heterocycle optionally substituted with 1, 2, or 3 substituents independently selected from R⁴⁰ .

A254. The compound of any one of embodiments A166-A250, wherein R²³ is aryl optionally substituted with 1, 2, or 3 substituents independently selected from R⁴⁰.

A255. The compound of any one of embodiments A166-A250, wherein R²³ is phenyl optionally substituted with 1, 2, or 3 substituents independently selected from R⁴⁰.

A256. The compound of any one of embodiments A166-A250, wherein R²³ is bicycle optionally substituted with 1, 2, or 3 substituents independently selected from R⁴⁰.

A257. The compound of any one of embodiments A166-A221, wherein Linker is of formula:

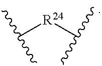

A258. The compound of any one of embodiments A166-A257, wherein R²⁴ is bond.

A259. The compound of any one of embodiments A166-A257, wherein R²⁴ is CH₂.

A260. The compound of any one of embodiments A166-A257, wherein R²⁴ is heterocycle optionally substituted with 1, 2, or 3 substituents independently selected from R⁴⁰ .

A261. The compound of any one of embodiments A166-A257, wherein R²⁴ is aryl optionally substituted with 1, 2, or 3 substituents independently selected from R⁴⁰.

A262. The compound of any one of embodiments A166-A257, wherein R²⁴ is phenyl optionally substituted with 1, 2, or 3 substituents independently selected from R⁴⁰.

A263. The compound of any one of embodiments A166-A257, wherein R²⁴ is bicycle optionally substituted with 1, 2, or 3 substituents independently selected from R⁴⁰.

A264. The compound of any one of embodiments A166-A257, wherein R²⁴ is C(O).

A265. The compound of any one of embodiments A166-A221, wherein the Linker is selected from the group consisting of: NA N N<N N N

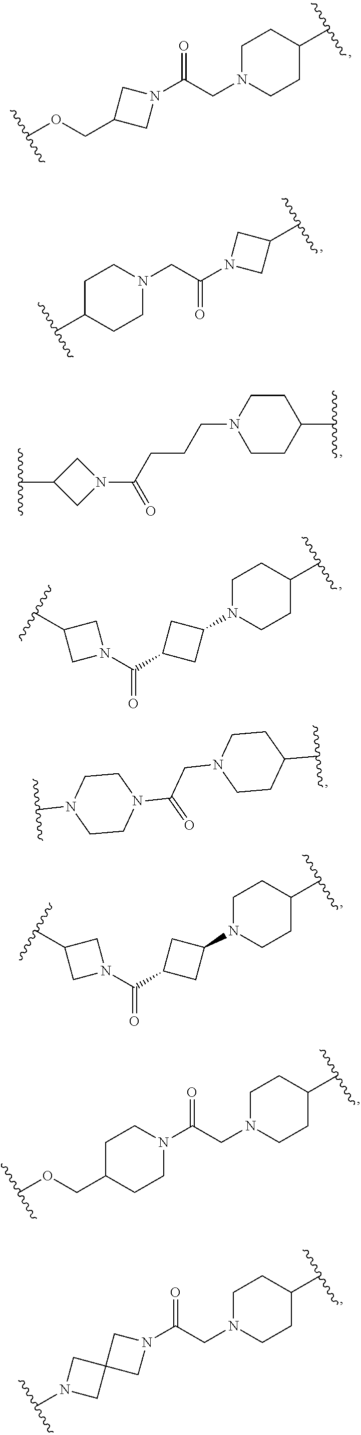

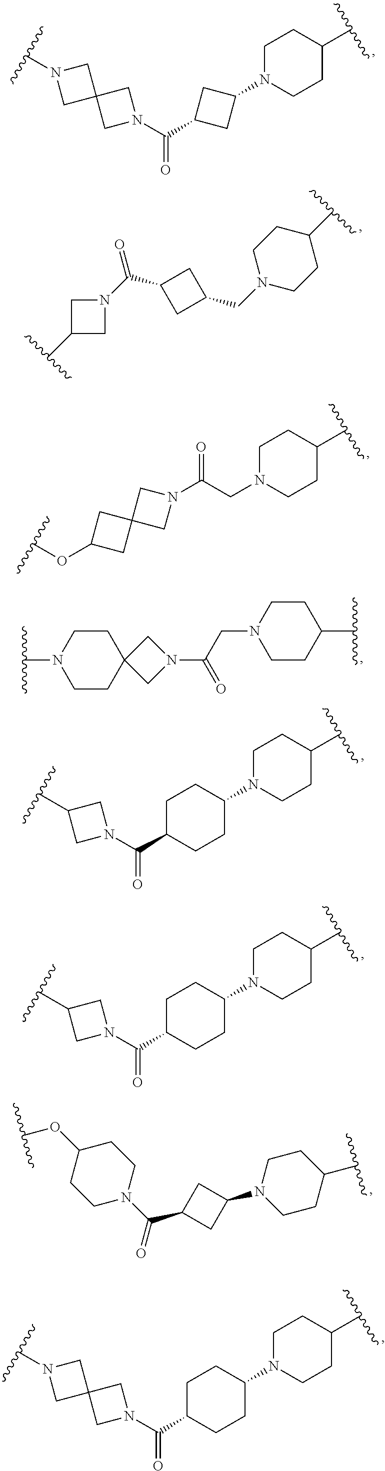

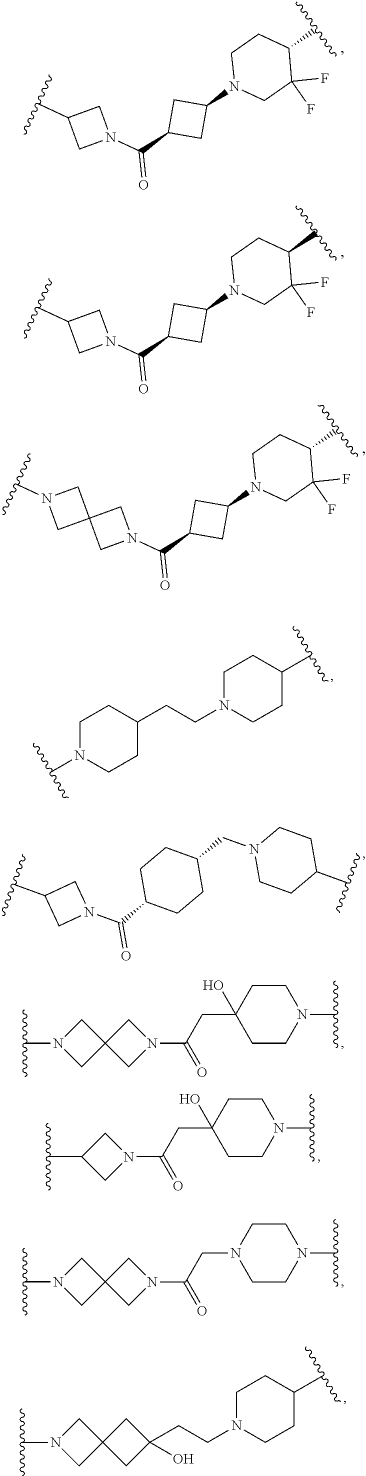

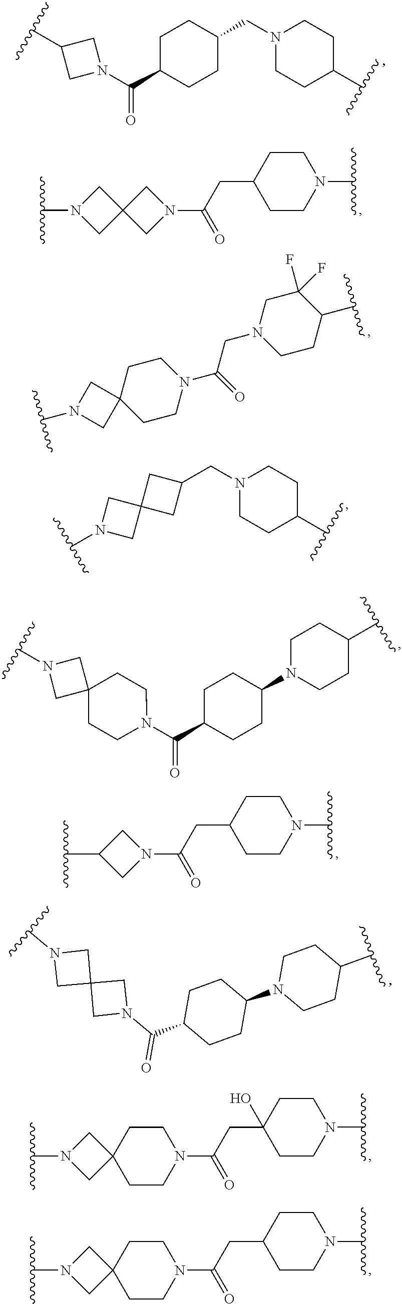

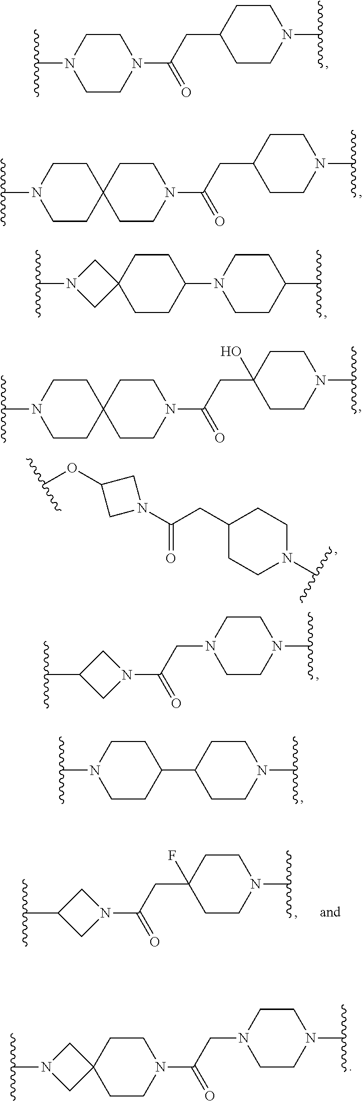

A266. The compound of any one of embodiments A166-A265, wherein the Targeting Ligand is a ligand that binds to a zinc finger protein.

A267. The compound of any one of embodiments A166-A265, wherein the Targeting Ligand is a ligand that binds to a Target Protein, wherein the Target Protein is selected from: ABL, AKT1, AKT2, ALK, androgen receptor, APi, AP2, ASH1L, ATAD2, ATF2, Aurora A, Aurora B, Aurora C, AXL, BAZ2A, BAZ2B, Bcl-2, BCL6, Bcl-XL, BCR-ABL, BMX, BRAF, BRD4, BRD9, BRPF1, cathepsin, CBP, CECR2, CSF1R, cyclin dependent kinase, DDR1, dihydrofolate reductase, DOT1L, EED, EGFR, EHMT1, EHMT2, EPHA2, EPHA3, EPHA4, EPHA7, EPHB4, estrogen receptor, EZH2, factor Xa, fatty acid binding protein, FES, FGFR2, FGFR4, FKBP, FLAP, FLT3, FYN, GSG2, HCK, HDM2, heat shock protein, histone acetyltransferase, IDO1, IDH1, IGF1R, INSR, ITK, kallikrein 7, KDM4, KDM5, KDM6, KIT, KSR1, L3MBTL3, lactoylglutathione lyase, LCK, LRRK2, LSD1, LYN, lysine methyltransferase, lysine-specific histone demethylase, mast/stem cell growth factor receptor, MCL-1, MDM2, MDM4, MEK1, MEN1, MER, MERTK, MET, mPGES-1, MST1R, MTH1, NTRK1, NTRK2, NTRK3, PAK1, PAK4, PDGFRA, PDGFRB, PHIP, PI3Ka, PPAR-gamma, protein S100-A7, RET, ROS1 receptor, Sec7, SETD2, SETD7, SETD8, SETDB1, SH2 domain, SMARCA2, SMYD2, SMYD3, SUV4-20H1, TAF1, TAF1L, TANK1, tie 2 receptor, TIFla, TNIK, mTORC1, mTORC2, TRKB, TRIM24, VEGF receptor, and YES1.

A268. The compound of any one of embodiments A166-A265, wherein the Targeting Ligand is a ligand that binds to a Target Protein, wherein the Target Protein is the androgen receptor. 10 A269. A pharmaceutical composition comprising a compound of any one of embodiments A166-A268 or a pharmaceutically acceptable salt thereof and a pharmaceutically acceptable carrier.

A270. A method of treating a disorder that is mediated by a Target Protein in a patient in need thereof comprising administering an effective amount of a compound of any one of embodiments A166-A268 or a pharmaceutical composition of embodiment A269.

IV. TARGET PROTEINS Degradation of cellular proteins is required for cell homeostasis and normal cell 20 function, such as proliferation, differentiation and cell death. When this system becomes dysfunctional or does not identify and abate abnormal protein behavior in vivo, a disease state can arise in a host, such as a human. A large range of proteins can cause, modulate or amplify diseases in vivo, as well known to those skilled in the art, published in literature and patent filings as well as presented in scientific presentations.

Therefore, in some embodiments, a selected Degron compound of the present invention can be administered in an effective amount to a host in need thereof to degrade a Target Protein that mediates a disorder to be treated. The selected protein target for degradation with a Degron may modulate a disorder in a human via a mechanism of action such as modification of a biological pathway, pathogenic signaling or modulation of a signal cascade or cellular entry.

In other embodiments, a selected Degrader compound of the present invention can be administered in an effective amount to a host in need thereof to degrade a Target Protein that mediates a disorder to be treated. The selected protein target may modulate a disorder in a human via a mechanism of action such as modification of a biological pathway, pathogenic signaling or modulation of a signal cascade or cellular entry.

In certain embodiments, the Target Protein is a protein that is not druggable in the classic sense in that it does not have a binding pocket or an active site that can be inhibited or otherwise bound, and cannot be easily allosterically controlled. In another embodiment, the Target Protein is a protein that is druggable in the classic sense, yet for therapeutic purposes, degradation of the protein is preferred to inhibition.

The Target Protein is recruited with a Targeting Ligand, which is a ligand for the Target Protein. Typically, the Targeting Ligand binds the Target Protein in a non-covalent fashion. In another embodiment, the Target Protein is covalently bound to the Degron in a manner that can be irreversible or reversible.

In some embodiments, the selected Target Protein is expressed from a gene that has undergone an amplification, translocation, deletion, or inversion event which causes or is caused by a medical disorder. In certain aspects, the selected Target Protein has been post-translationally modified by one, or a combination, of phosphorylation, acetylation, acylation including propionylation and crotylation, N-linked glycosylation, amidation, hydroxylation, methylation and poly-methylation, O-linked glycosylation, pyroglutamoylation, myristoylation, farnesylation, geranylgeranylation, ubiquitination, sumoylation, or sulfation which causes or is caused by a medical disorder.

As contemplated herein, the present invention includes a Degrader with a Targeting Ligand that binds to a Target Protein of interest. The Target Protein is any amino acid sequence to which a Degrader can be bound which by degradation thereof, causes a beneficial therapeutic effect in vivo.

In certain embodiments, the Target Protein is a non-endogenous peptide such as that from a pathogen or toxin. In another embodiment, the Target Protein can be an endogenous protein that mediates a disorder. The endogenous protein can be either the normal form of the protein or an aberrant form. For example, the Target Protein can be a mutant protein found in cancer cells, or a protein, for example, where a partial, or full, gain-of-function or loss-of-function is encoded by nucleotide polymorphisms. In some embodiments, the Degrader targets the aberrant form of the protein and not the normal form of the protein.

In another embodiment, the Target Protein can mediate an inflammatory disorder or an immune disorder, including an auto-immune disorder.

In certain embodiments, the Target Protein is a non-endogenous protein from a virus.

Non-limiting examples of non-endogenous proteins include HIV, HBV, HCV, RSV, HPV, CMV, SARS-CoV2, flavivirus, pestivirus, coronavirus, and noroviridae.

In certain embodiments, the Target Protein is a non-endogenous protein from a bacteria, which may be for example, a gram positive bacteria, gram negative bacteria or other, and can be a drug-resistant form of bacteria.

In certain embodiments, the Target Protein is a non-endogenous protein from a fungus. In one embodiment, the Target Protein is a non-endogenous protein from a prion. In one embodiment, the Target Protein is a protein derived from a eukaryotic pathogen, for example a protist, helminth, etc.

In certain aspects, the Target Protein mediates chromatin structure and function. The Target Protein may mediate an epigenetic action such as DNA methylation or covalent modification of histones. An example is histone deacetylase (HDAC 1, 2, 3, 4, 5, 6, 7, 8, 9, 10 or 11). Alternatively, the Target Protein may be a bromodomain, which are readers of lysine acetylation (for example, BRD1, 2, 3, 4, 5, 6, 7, 8, 9 and T). FIG. 9 illustrates the proteins of the bromodomain family, which, for example, can act as Target Proteins according to the present invention.

Other nonlimiting examples of Target Proteins are a structural protein, receptor, enzyme, cell surface protein, a protein involved in apoptotic signaling, aromatase, helicase, mediator of a metabolic process (anabolism or catabolism), antioxidant, protease, kinase, oxidoreductase, transferase, hydrolase, lyase, isomerase, ligase, enzyme regulator, signal transducer, structural molecule, binding activity (protein, lipid carbohydrate), cell motility protein, membrane fusion protein, cell communication mediator, regulator of biological processes, behavioral protein, cell adhesion protein, protein involved in cell death, protein involved in transport (including protein transporter activity, nuclear transport, ion transporter, channel transporter, carrier activity, permease, secretase or secretion mediator, electron transporter, chaperone regulator, nucleic acid binding, transcription regulator, extracellular organization and biogenesis regulator, and translation regulator).

In some embodiments, the Target Protein is a modulator of a signaling cascade related to a known disease state. In another embodiment, the Target Protein mediates a disorder by a mechanism different from modulating a signaling cascade. Any protein in a eukaryotic system or a microbial system, including a virus, bacteria or fungus, as otherwise described herein, are targets for proteasomal degradation using the present invention. The Target Protein may be a eukaryotic protein, and in some embodiments, a human protein.

In certain embodiments, the Target Protein is RXR, DHFR, Hsp90, a kinase, HDM2, MDM2, BET bromodomain-containing protein, HDAC, IDH1, Mcl-1, human lysine methyltransferase, a nuclear hormone receptor, aryl hydrocarbon receptor (AHR), RAS, RAF, FLT, SMARC, KSR, NF2L, CTNB, CBLB, BCL.

In one embodiment, a bromodomain containing protein has histone acetyl transferase activity.

In one embodiment, the bromodomain containing protein is BRD2, BRD3, BRD4, BRDT or ASH1L.

In certain embodiments, the bromodomain containing protein is a non-BET protein.

In certain embodiments, the non-BET protein is BRD7 or BRD9.

The present invention may be used to treat a wide range of disease states and/or conditions, including any disease state and/or condition in which a protein is dysregulated and where a patient would benefit from the degradation of proteins.

For example, a Target Protein can be selected that is a known target for a human therapeutic, and the therapeutic can be used as the Targeting Ligand when incorporated into the Degrader according to the present invention. These include proteins which may be used to restore function in a polygenic disease, including for example B7.1 and B7, TINFRlm, TNFR2, NADPH oxidase, Bcl2/Bax and other partners in the apoptosis pathway, C₅a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, e.g.., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuraminidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+channels, VCAM, VLA-4 integrin, selectins, CD40/CD40L, neurokinins and receptors, inosine monophosphate dehydrogenase, p38 MAP Kinase, Ras/Raf/MER/ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-2/neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further Target Proteins include Acetyl-CoA carboxylase, adenylosuccinate synthetase, protoporphyrinogen oxidase, and enolpyruvylshikimate-phosphate synthase.

In certain embodiments, the Target Protein is derived from a kinase to which the Targeting Ligand is capable of binding or binds including, but not limited to, a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIEl, TNK1, TNK2, TNNI3K, TXK, TYK2, TYRO3, YES1, or ZAP70).

In certain embodiments, the Target Protein is derived from a kinase to which the Targeting Ligand is capable of binding or binds including, but not limited to, a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2).

In certain embodiments, the Target Protein is derived from a kinase to which the Targeting Ligand is capable of binding or binds including, but not limited to a cyclin dependent kinase for example CDK1, CDK2, CDK3, CDK4, CDK5, CDK6, CDK7, CDK8, CDK9, CDK10, CDK11, CDK12, or CDK13.

In certain embodiments, the Target Protein is derived from a kinase to which the Targeting Ligand is capable of binding or binds including, but not limited to a leucine-rich repeat kinase (e.g., LRRK2).

In certain embodiments, the Target Protein is derived from a kinase to which the Targeting Ligand is capable of binding or binds including, but not limited to a lipid kinase (e.g., PIK3CA, PIK3CB) or a sphingosine kinase (e.g. SiP).

In certain embodiments, the Target Protein is derived from a BET bromodomain-containing protein to which the Targeting Ligand is capable of binding or binds including, but not limited to, ASH1L, ATAD2, BAZ1A, BAZ1B, BAZ2A, BAZ2B, BRD1, BRD2, BRD3, BRD4, BRD5, BRD6, BRD7, BRD8, BRD9, BRD10, BRDT, BRPF1, BRPF3, BRWD3, CECR2, CREBBP, EP300, FALZ, GCN5L2, KIAA1240, LOC93349, MLL, PB1, PCAF, PHIP, PRKCBP1, SMARCA2, SMARCA4, SP100, SP110, SP140, TAF1, TAF1L, TIFla, TRIM28, TRIM33, TRIM66, WDR9, ZMYND 11, and MLL4. In certain embodiments, a BET bromodomain-containing protein is BRD4.

In certain embodiments, the Target Protein is derived from a nuclear protein to which the Targeting Ligand is capable of binding or binds including, but not limited to, BRD2, BRD3, BRD4, Antennapedia Homeodomain Protein, BRCA1, BRCA2, CCAAT-Enhanced-Binding Proteins, histones, Polycomb-group proteins, High Mobility Group Proteins, Telomere Binding Proteins, FANCA, FANCD2, FANCE, FANCF, hepatocyte nuclear factors, Mad2, NF-kappa B, Nuclear Receptor Coactivators, CREB-binding protein, p55, p107, p130, Rb proteins, p53, c-fos, c-jun, c-mdm2, c-myc, and c-rel.

In certain embodiments, the Target Protein is a member of the Retinoid X Receptor (RXR) family and the disorder treated is a neuropsychiatric or neurodegenerative disorder. In certain embodiments, the Target Protein is a member of the Retinoid X Receptor (RXR) family and the disorder treated is schizophrenia.

In certain embodiments, the Target Protein is dihydrofolate reductase (DHFR) and the disorder treated is cancer. In certain embodiments, the Target Protein is dihydrofolate reductase (DHFR) and the disorder treated is microbial.

In certain embodiments, the Target Protein is dihydrofolate reductase from bacillus Anthracis (BaDHFR) and the disorder treated is anthrax.

In certain embodiments, the Target Protein is Heat Shock Protein 90 (HSP90) and the disorder treated is cancer.

In certain embodiments, the Target Protein is a kinase or phosphatase and the disorder treated is cancer.

In certain embodiments, the Target Protein is HDM2 and or MDM2 and the disorder treated is cancer.

In certain embodiments, the Target Protein is a BET bromodomain containing protein and the disorder treated is cancer.

In certain embodiments, the Target Protein is a lysine methyltransferase and the disorder treated is cancer.

In certain embodiments, the Target Protein belongs to the RAF family and the disorder treated is cancer.

In certain embodiments, the Target Protein belongs to the FKBP family and the disorder treated is an autoimmune disorder. In certain embodiments, the Target Protein belongs to the FKBP family and the disorder treated is organ rejection. In certain embodiments, the Target Protein belongs to the FKBP family and the compound is given prophylactically to prevent organ failure.

In certain embodiments, the Target Protein is an androgen receptor and the disorder treated is cancer.

In certain embodiments, the Target Protein is an estrogen receptor and the disorder treated is cancer.

In certain embodiments, the Target Protein is a viral protein and the disorder treated is a viral infection. In certain embodiments, the Target Protein is a viral protein and the disorder treated is HIV, HPV, SARS-CoV2, or HCV.

In certain embodiments, the Target Protein is an AP-1 or AP-2 transcription factor and the disorder treated is cancer.

In certain embodiments, the Target Protein is a HIV protease and the disorder treated is a HIV infection. In certain embodiments, the Target Protein is a HIV integrase and the disorder treated is a HIV infection. In certain embodiments, the Target Protein is a HCV protease and the disorder treated is a HCV infection. In certain embodiments, the treatment is prophylactic and the Target Protein is a viral protein.

In certain embodiments, the Target Protein is a member of the histone deacetylase (HDAC) family and the disorder is a neurodegenerative disorder. In certain embodiments, the Target Protein is a member of the histone deacetylase (HDAC) family and the disorder is Huntingon's, Parkinson's, Kennedy disease, amyotropic lateral sclerosis, Rubinstein-Taybi syndrome, or stroke.

In certain embodiments, Targeting Ligand forms a covalent bond with the Target Protein. Non-limiting examples of Target Proteins and Targeting Ligands utilizing a covalent bond include those described in “Covalent Inhibitors Design and Discovery” Eur J Med Chem. 2017 Sep. 29; 138:96-114. doi: 10.1016/j.ejmech.2017.06.019;. “Lysine-Targeting Covalent Inhibitors.” Angew Chem Int Ed Engl. 2017 Aug 29. doi: 10.1002/anie.201707630; “Inhibition of Mcl-1 Through Covalent Modification of a Noncatalytic Lysine Side Chain.” Nat Chem Biol. 2016 November; 12(11):931-936; “Proteome-wide Map of Targets of T790M-EGFR-Directed Covalent Inhibitors” Cell Chem. Biol. 2016 Nov: 24:1-13; “Global Profiling of Lysine Reactivity and Ligandability in the Human Proteome” Nat. Chem. 2017 Jul 31, doi:10.1038/nchem.2826; “The Resurgence of Covalent Drugs” Nat. Rev. Drug Disc. 2011 10, 307-217; U.S. Pat. Nos. 8,008,309; and 9,790,226.

In other embodiments, the Target Protein is selected from DOTL1, CBP, WDR5, BRAF, KRAS, MCL1, PTPN2, HER2, and SHOC2. In another embodiment, the Target Protein is selected from UCHL1, USP6, USP14, and USP30. In another embodiment, the Target Protein is selected from USP1, USP2, USP4, USP6, USP7, USP8, USP9x, USP10, USP 11, USP13, USP14, USP17, and USP28.

In some embodiments, the Target Protein is selected from 4QL1, 3SMR, 5EAL, 6DAK, 6DAR, and 6DAS.

In certain embodiments, the Target Protein as referred to herein is named by the gene that expresses it. The person skilled in the art will recognize that when a gene is referred to as a Target Protein, the protein encoded by the gene is the Target Protein. For example, ligands for the protein SMCA2 which is encoded by SMRCA2 are referred to as SMRCA2 Targeting Ligands.

Degradation of target proteins through “molecular glue” interactions have been investigated by many researchers. An example review of the field is Dong et al. “Molecular Glues for Targeted Protein Degradation: From Serendipity to Rational Discovery” J. Med. 25 Chem. 2021, 64, 15, 10606-10620. See also, for example, Cao et al. “Defining molecular glues with a dual-nanobody cannabidiol sensor” Nature Communications, 2022, 13:815.

In certain embodiments a degron of the present invention degrades the target protein using a molecular glue mechanism. Research in the area of targeted protein degradation includes systemic studies of the “degrome” of zinc finger proteins. The C2H2 zinc finger 30 degrome is present on several different target proteins (See, for example: Sievers et al. “Defining the human C2H2 zinc finger degrome targeted by thalidomide analogs through CRBN” Science, 2018, 362, eaat0572).

In certain embodiments, the Target Protein is ARID2. Yamamoto has shown that ARID2 can be a substrate for targeted protein degradation. ARID2 has been associated with poor prognosis and chemoresistant residual disease in multiple myeloma (Yamamoto et al. “ARID2 is a pomalidomide-dependent CRL4CRBN substrate in multiple myeloma cells” Nature Chemical Biology, 2020; 16, 1208-1217.

In certain embodiments, the Target Protein is aromatase. Aromatase has been identified as a substrate of cereblon and may be related to thrombocytopenia induced by treatment with pomalidomide or lenalidomide. See for example, Tochigi et al. “Aromatase is a novel neo-substrate of cereblon responsible for immunomodulatory drugs-induced thrombocytopenia” Blood (2020); 135, 24, 2146-2158. In certain embodiments a Degron described herein does not have aromatase degradation activity.

In certain embodiments, the Target Protein is CDK12. CDK12 binding with a degrading compound has been shown to facilitate the degradation of cyclin K. Degradation of cyclin K may be useful in the treatment of proliferative diseases such as cancer. See for example, Mayor-Ruiz et al. “Rational discovery of molecular glue degraders via scalable chemical profiling” Nature Chemical Biology, 2020, 16, 1199-1207; Slabicki et al. “The CDK8 inhibitor CR8 acts as a molecular glue degrader that depletes cyclin K”, Nature, 2020, 585, 293-297; and Lv et al. “discovery of a molecular glue promoting CDK12-DDB1 interaction to trigger Cyclin K degradation” BioRxiv, 2020; doi:10.1101/2020.06.10.144303.

In certain embodiments, the Target Protein is casein kinase la (CKla). CKla degradation has been associated with myelodysplastic syndrome. Targeted protein degradation of CKla has been shown by Teng et al. “Development of PDE6D and CKla Degraders through Chemical Derivatization of FPFT-2216” J. Med. Chem. 2022 65, 1, 747-756.

In certain embodiments, the Target Protein is p63. The teratogenic effects of thalidomide have been associated with degradation of p63 isoforms. Compounds for the degradation of TAp63a and ANp63a have been developed. See, for example, Asatsyna-Okumura et al. “p63 is a cereblon substrate involved in thalidomide teratogenicity” Nature Chemical Biology, 2019, 15, 1077-1084.

In certain embodiments, the Target Protein is G1 to S phase transition protein (GSPT1).

Targeted degradation has been demonstrated to lower GSPT1 levels, exhibiting an antiproliferative effect. See, for example, Powell et al. “Selective Degradation of GSPT1 by Cereblon Modulators Identified via a Focused Combinatorial Library” ACS Chem Biol., 2020, 15, 2722-2730; and Hansen et al. “Protein Degradation via CRL4CRBN Ubiquitin Ligase: Discovery and Structure-Activity Relationships of Novel Glutarimide Analogs That Promote Degradation of Aiolos and/or GSPT1” J. Med. Chem. 2018, 61, 2, 492-503. In certain embodiments, the Target Protein is FAM83F. FAM83F has been implicated in oncogenesis, specifically esophageal squamous cell carcinoma, lung adenocarcinoma, glioma, and thyroid carcinoma. FAM83F degradation has been demonstrated to reduce the level of CKla. See, for example, Dunbar et al. “IMiDs induce FAM83F degradation via an interaction with CKla to attenuate Wnt signalling” BioRxiv, doi:10.1101/2020.05.25.114660.

In certain embodiments, the Target Protein is RBM39. Targeted degraders of RBM39 have been investigated in clinical studies involving solid tumors. Genetics studies indicate RBM39 may be involved in acute myeloid leukemia. Degradation of RBM39 is mediated though a molecular glue interaction with DCAF15. See, for example, Faust et al. “Structural complementarity facilitates E7820-mediated degradation of RBM39 by DCAF15”, Nature Chemical Biology, 2020, 16, 7-14.

In certain embodiments, the Target Protein is SALL4. SALL4 has been implicated as one of the targets for thalidomide which resulted in the teratogenic effects of thalidomide.

Hydroxythalidomide, a major metabolite of thalidomide, has been demonstrated to induce the degradation of SALL4 through complex formation with CRBN. For example, see Furihata et al. “Structural bases of IMiD selectivity that emerges by 5-hydroxythalidomide” Nature Communications, 2020, 11, 4748.

In certain embodiments, the Target Protein is interleukin enhancer-binding factor 2 (ILF2). ILF2 forms a heterodimer with ILF3 and regulates gene expression and cancer cell growth. Cancers regulated by ILF2 include breast cancer, gastric cancer, liver cancer, and non-small cell lung cancer. Targeted protein degraders of ILF2 have been shown to reliably induce the degradation of ILF2. See, for example, Lian et al. “Cereblon Promotes the Ubiquitination and Proteasomal Degradation of Interleukin Enhancer-Binding Factor 2” The Protein Journal, 2020, doi:10.1007/s10930-020-09918-9.

In certain embodiments, the Target Protein is promyelocytic leukemia zinc finger protein (PLZF and ZBTB16). ZBTB16, a CRBN neosubstrate, has been implicated in regulating spermatogenesis, immune function, and hematopoiesis. In particular, acute promyelocytic leukemia Targeted protein degraders of ZBTB16 have been disclosed in, for example, Matyskiela et al. “Cereblon Modulators Target ZBTB16 and Its Oncogenic Fusion Partners for Degradation via Distinct Structural Degrons” ACS Chem. Biol. 2020, 15, 12, 3149-3158.

In certain embodiments, the Target Protein is ZMYM2. ZMYM2 is a zinc-finger protein which has been implicated in a chimeric fusion gene with FGFR1. These fusions can lead to myeloid lymphoid neoplasms and acute leukemia. Researchers have developed compounds which target ZMYM2 and facilitate its degradation through ubiquitination. For example, see Renneville et al. “Avadomide Induces Degradation of ZMYM2 Fusion Oncoproteins in Hematologic Malignancies” Blood Cancer Discovery, 2021, 2, 250-265.

In other embodiments, the Target Protein is selected from ARID2, CDK1, CDK12-cyclin K, CDK13, CK1alpha, CSNK1A1, Cyclin K, E4F1, FAM83F, GSPT1, GSPT2, GZF1, IKZF1, IKZF2, IKZF3, IKZF4, ILF2, Myc, ODC1, p63, PDE6D, AB28, RARalpha-ZBTB16, RBM23, RBM39, RBM39, RNF166, SALL4, WBP4, ZBTB16, ZBTB16-RARalpha, ZBTB39, ZFP91, ZFP91, ZFP91, ZMYM2-FGFR1, ZMYM2-FLT3, ZNF198, ZNF276, ZNF276, ZNF517, ZNF582, ZNF653, ZNF654, ZNF692, ZNF787, ZNF827, and ZNF98.

In certain embodiments the Target Protein is selected from 4BVV, ABL1, ABL2, AKT1, AKT2, androgen receptor, APi, AP2, ASH1L, ATAD2, ATF2, AXL, BAZ2A, BAZ2B, Bcl-2, Bcl-XL, BCR-ABL, BMX, BRPF1, cathepsin, CECR2, CSF1R, cyclin dependent kinase, DDR1, dihydrofolate reductase, DOT1L, EED, EHMT1, EHMT2, EPHA2, EPHA3, EPHA4, EPHA7, EPHB4, estrogen receptor, EZH2, factor Xa, fatty acid binding protein, FES, FKBP, FLAP, FLT3, FYN, GSG2, HBV, HCK, HCV protease, HDM2, heat shock protein, histone acetyltransferase, HIV integrase, HIV protease, HIV reverse transcriptase, IDO1, IDH1, IGF1R, INSR, ITK, kallikrein 7, KDM4, KDM5, KDM6, KIT, kringle domain V, KSR1, L3MBTL3, lactoylglutathione lyase, LCK, LSD1, LYN, lysine methyltransferase, lysine-specific histone demethylase, mast/stem cell growth factor receptor, MCL-1, MDM2, MDM4, MEK1, MEN1, MER, MERTK, MET, mPGES-1, MST1R, MTH1, NTRK, PAK1, PAK4, PB1, PDGFR receptor, PDZ, PHIP, phospholipase A2 domain, PNET, PPAR-gamma, protein S100-A7, RAML receptor, RCC receptor, ROS1 receptor, saposin-B, Sec7, SEGA receptor, SETD2, SETD7, SETD8, SETDB1, SF6D, SH2 domain, SMYD2, SMYD3, SUV4-20H1, TAF1, TAF1L, TANK1, TEC, tie 2 receptor, TNIK, mTORC1, mTORC2, TRKB, TRIM24, U09-CX-5279, and VEGF receptor.

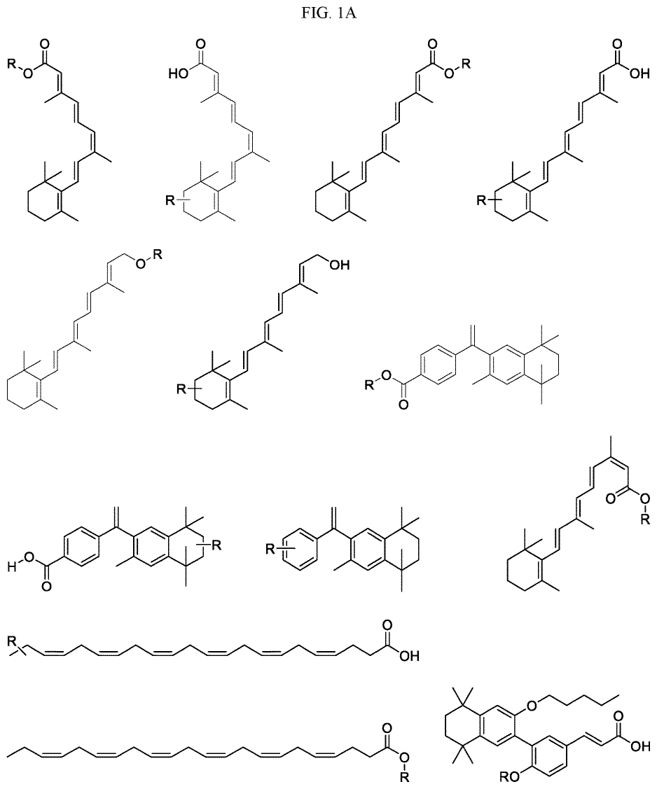

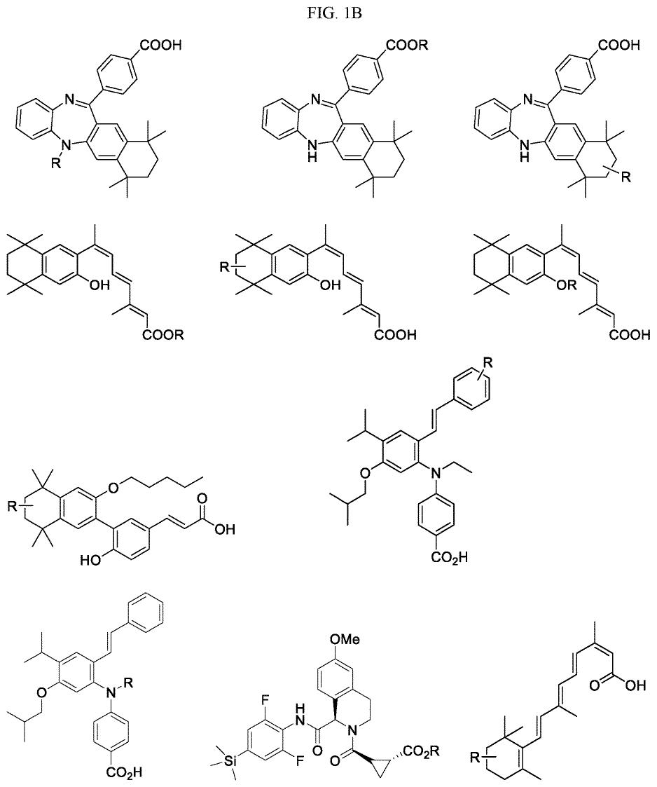

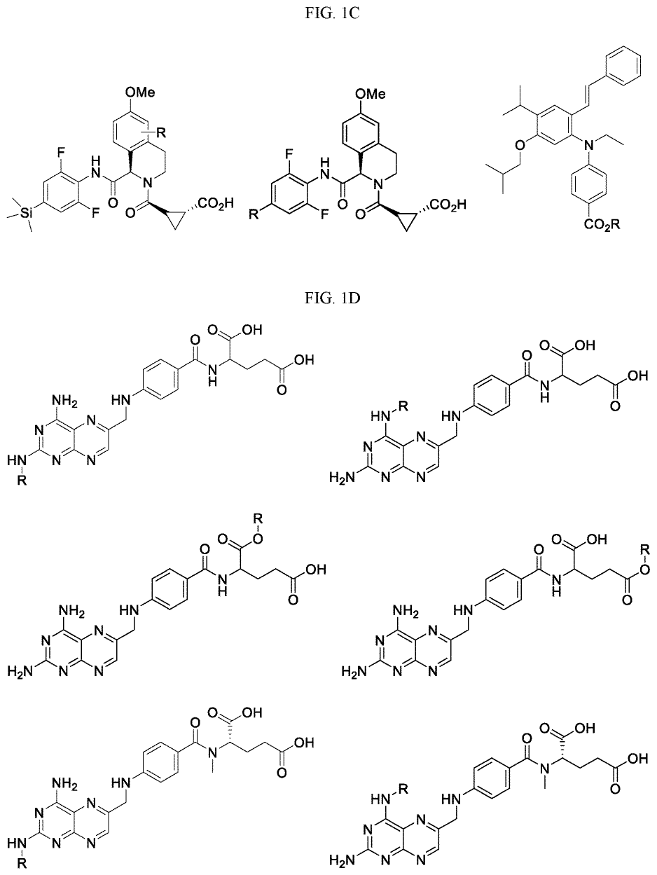

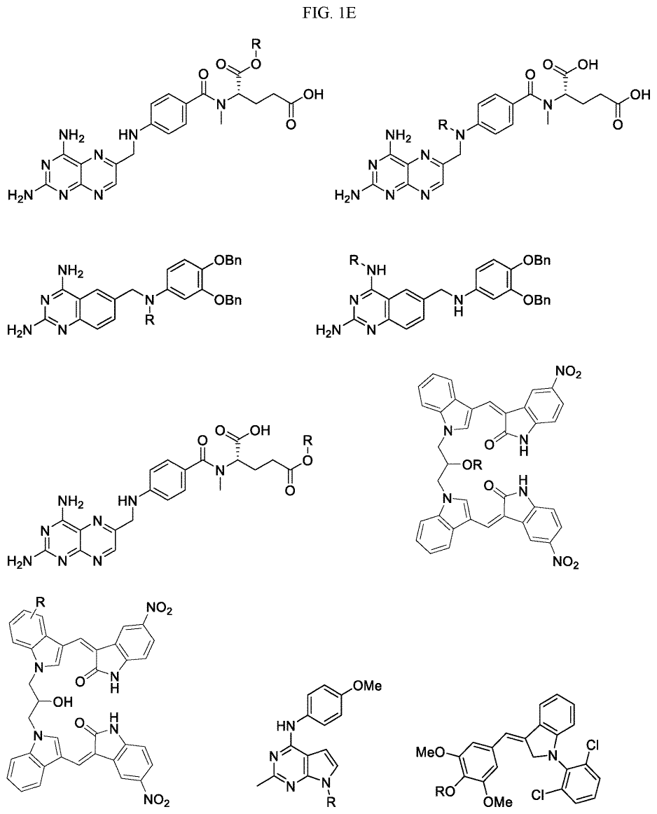

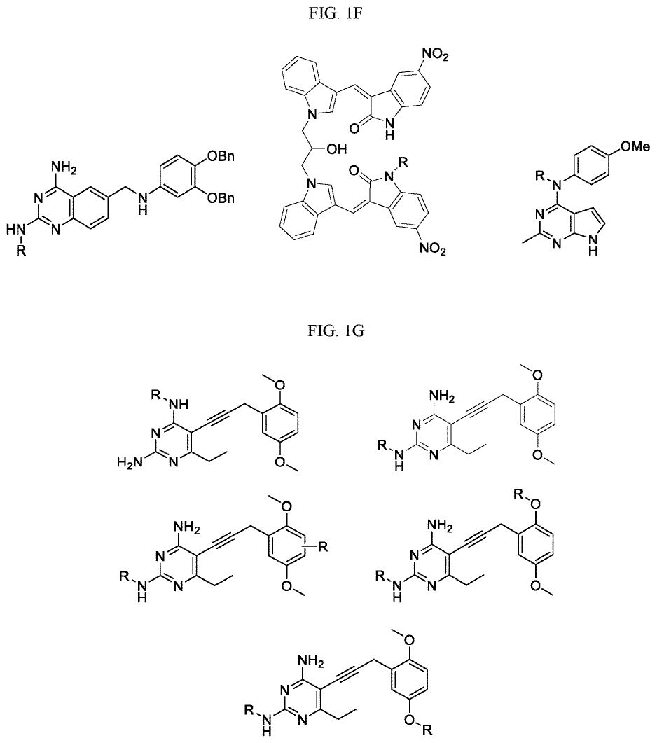

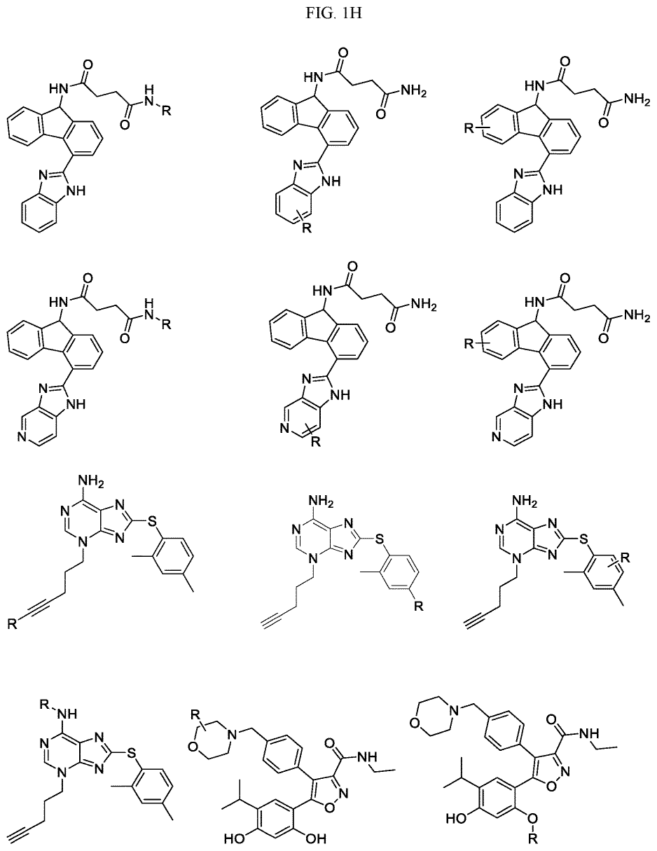

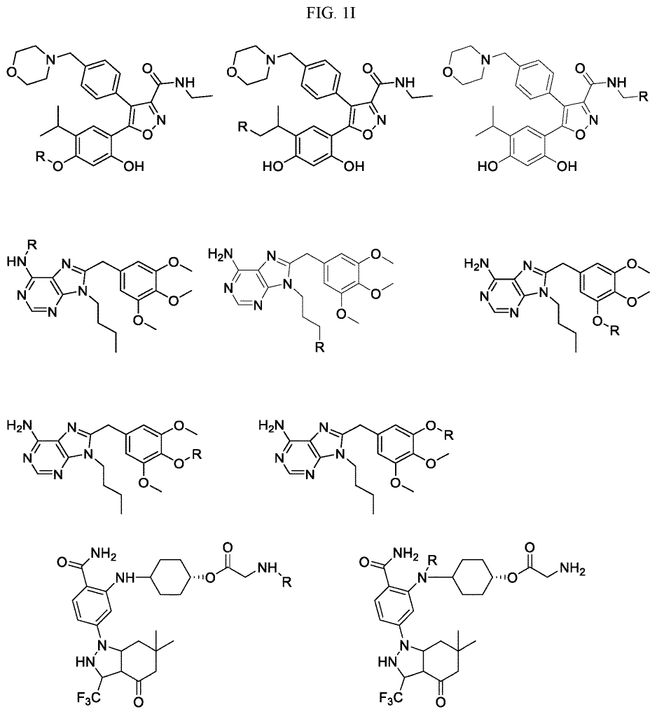

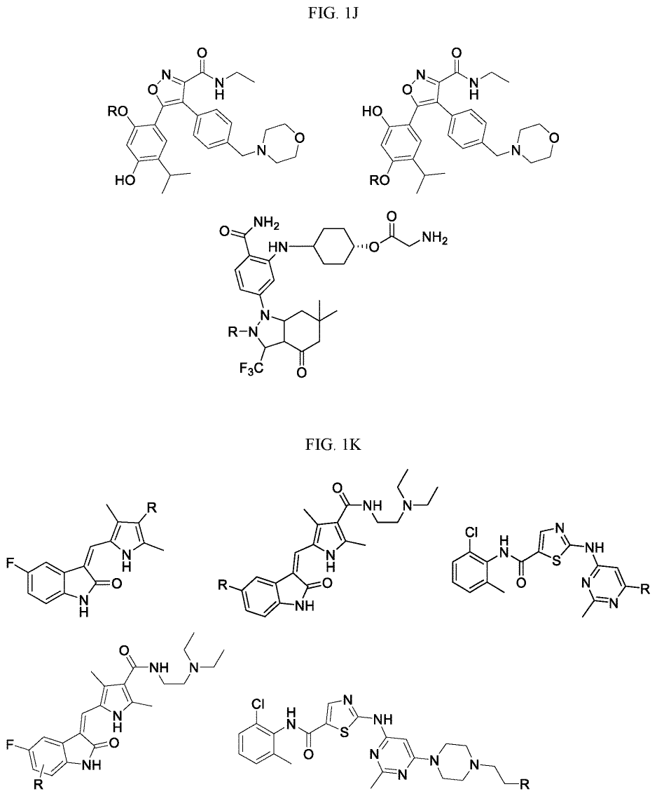

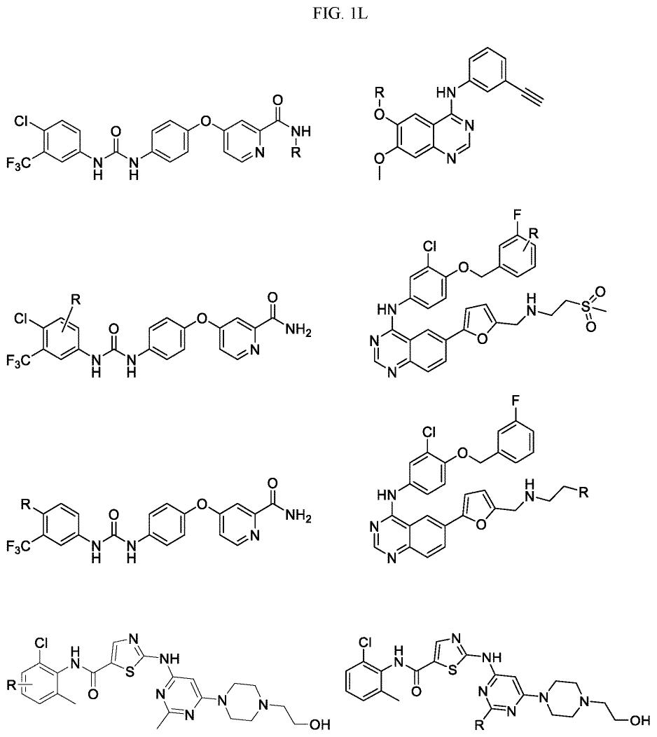

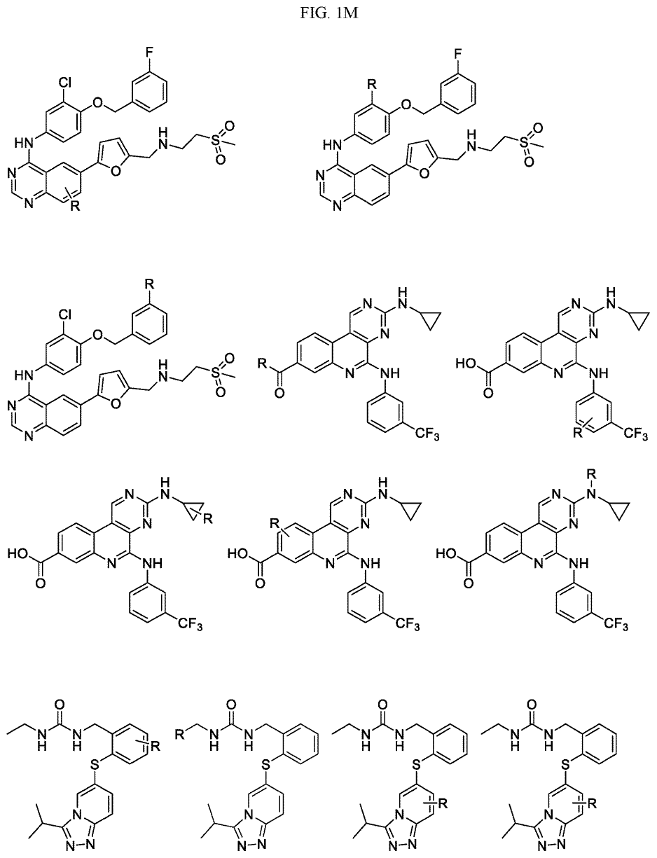

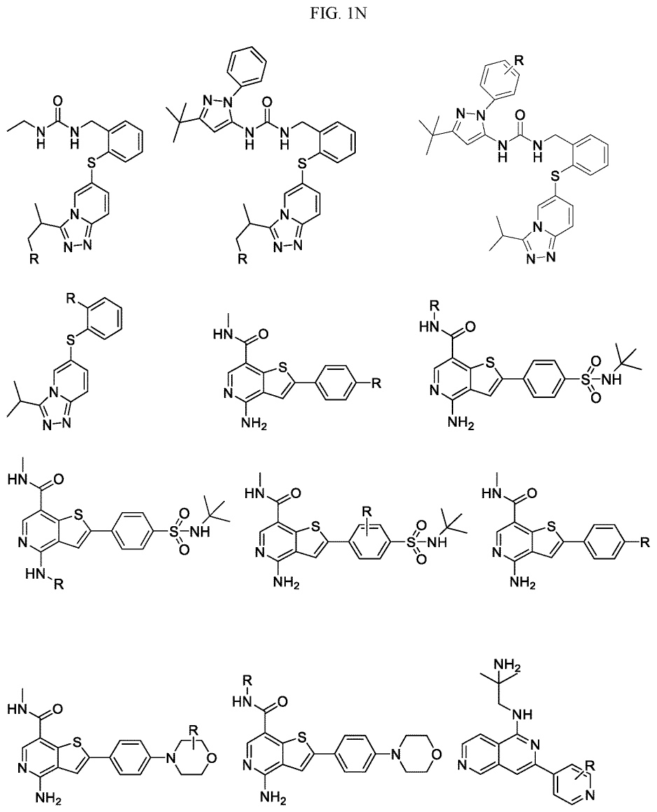

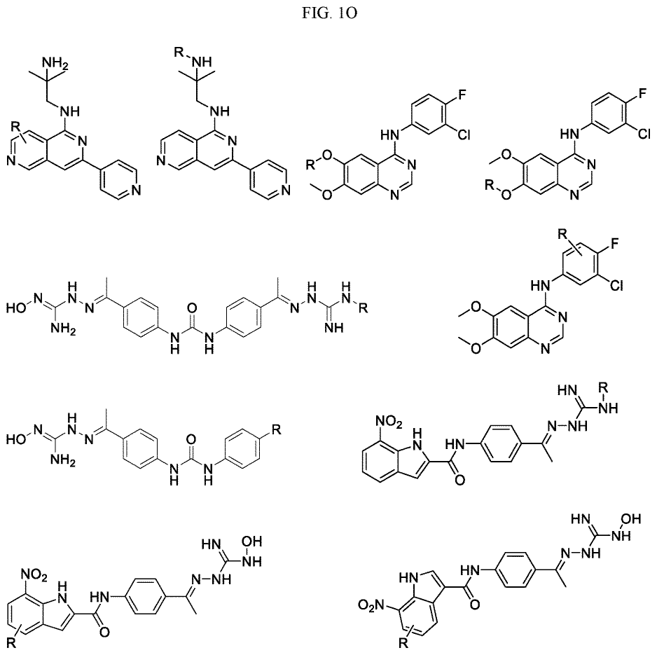

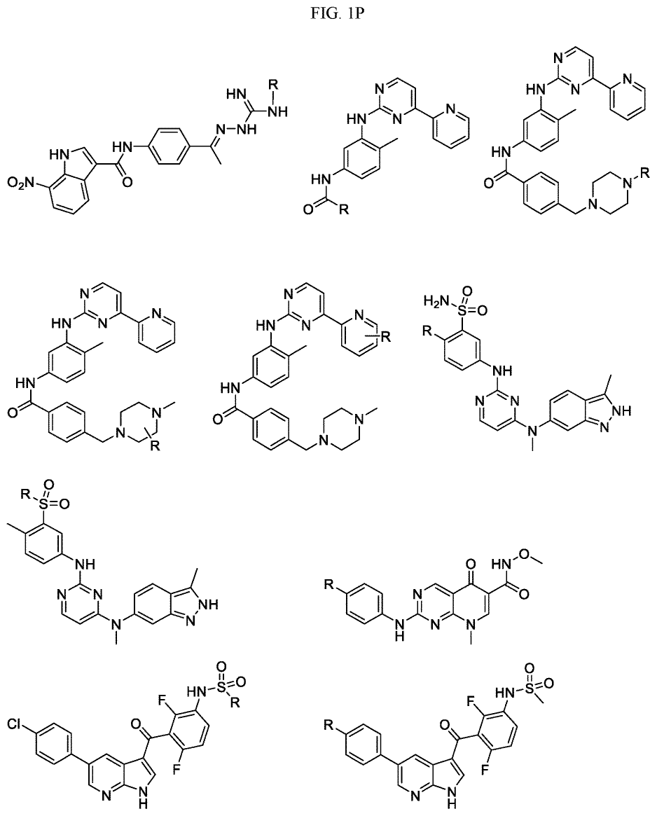

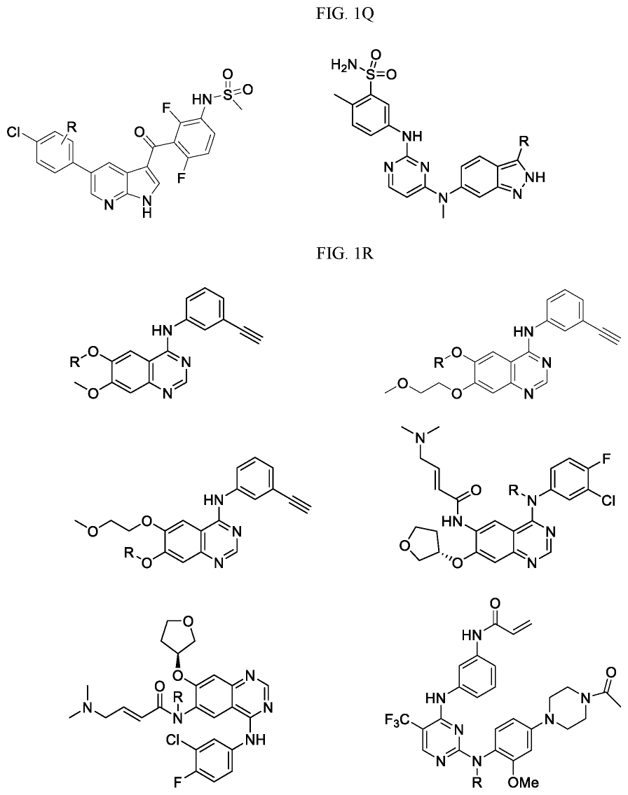

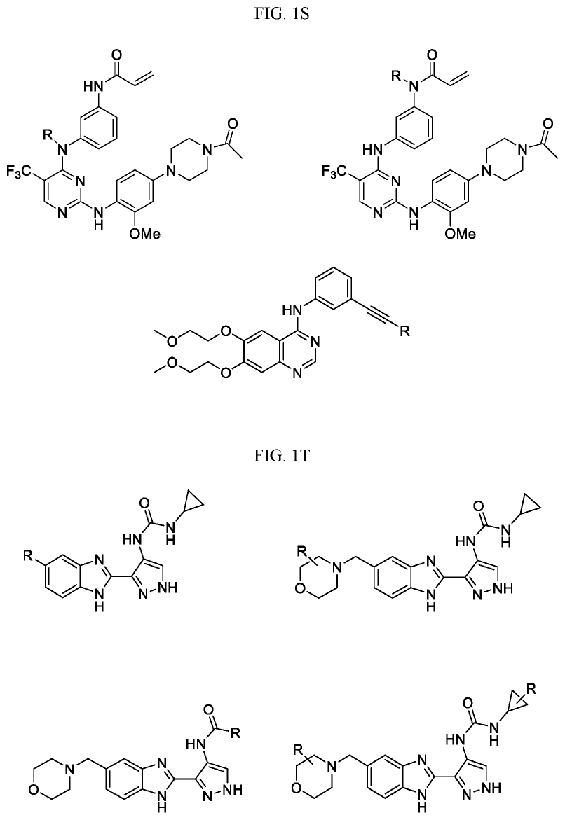

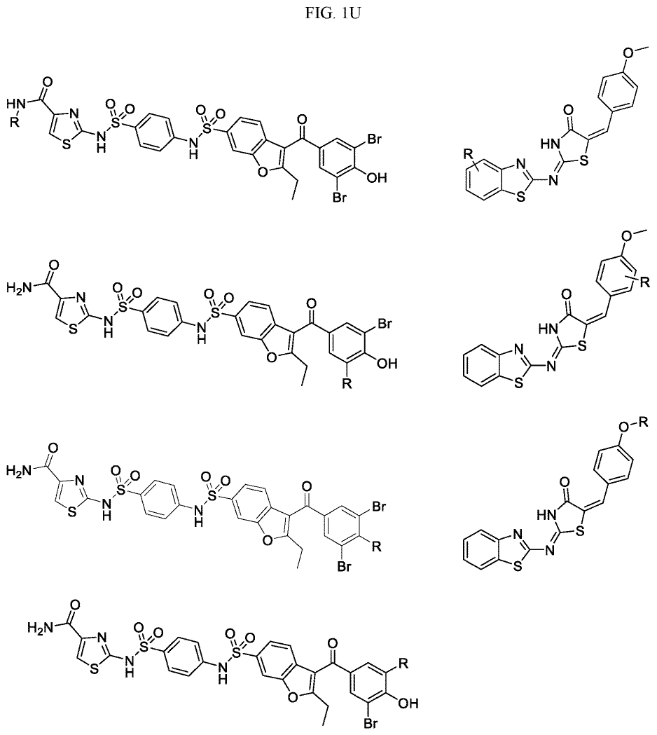

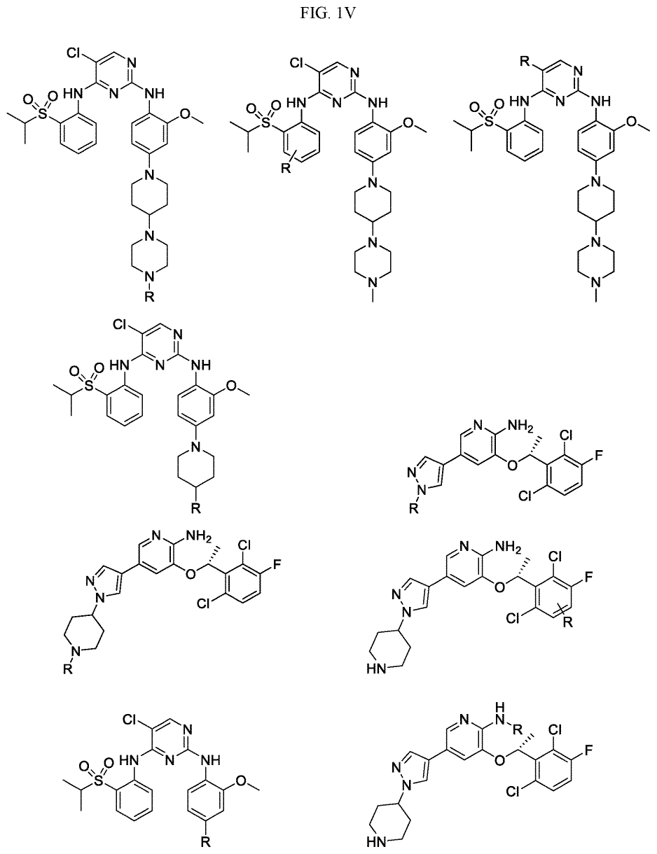

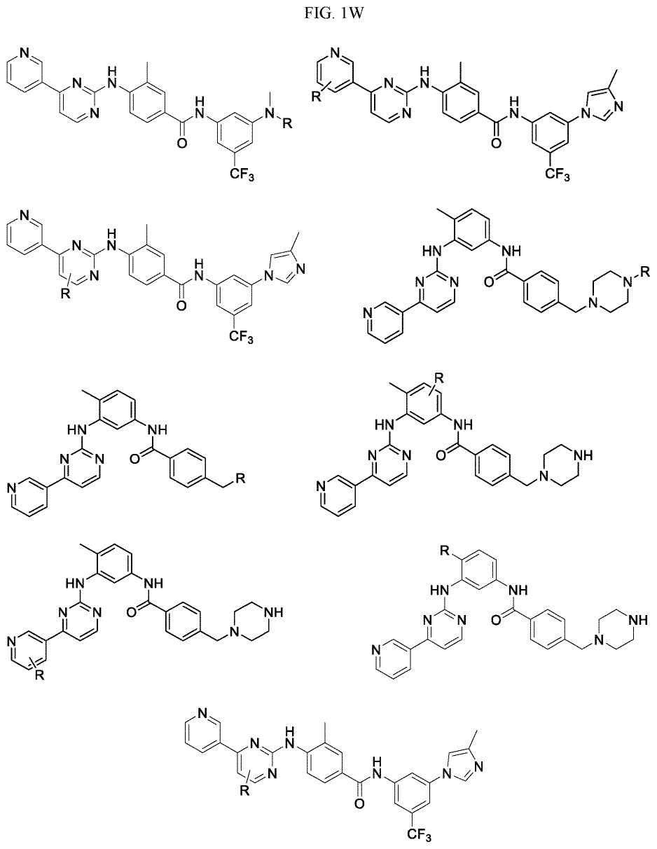

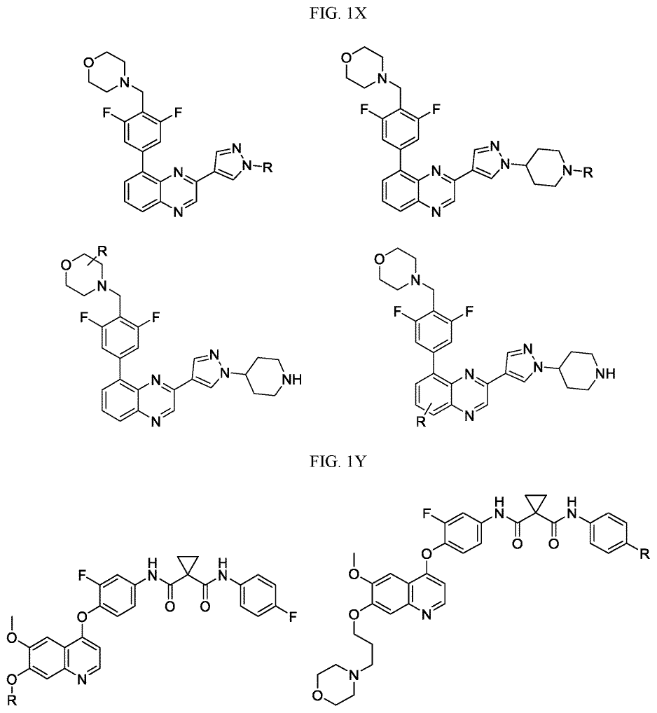

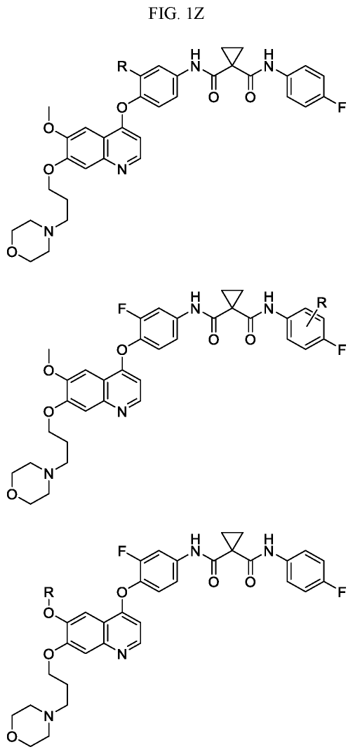

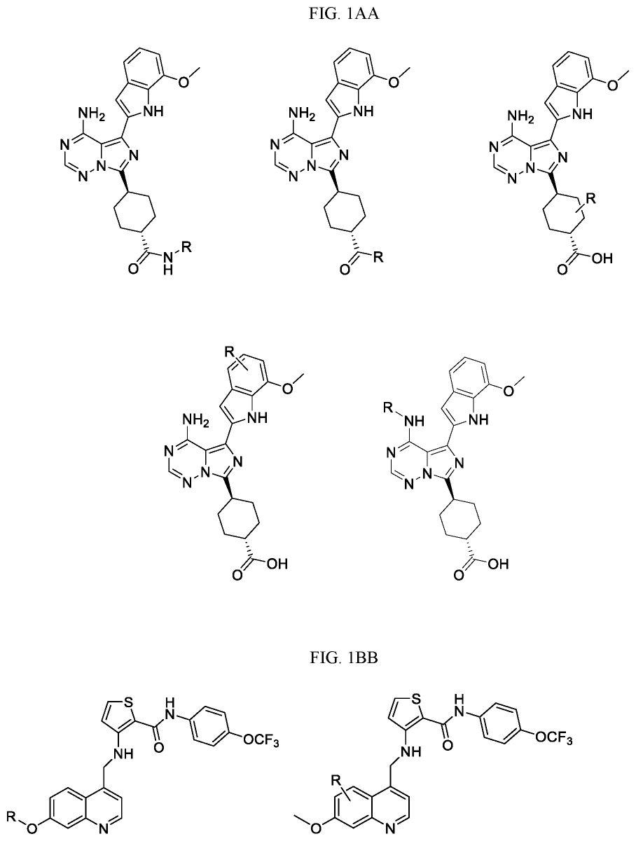

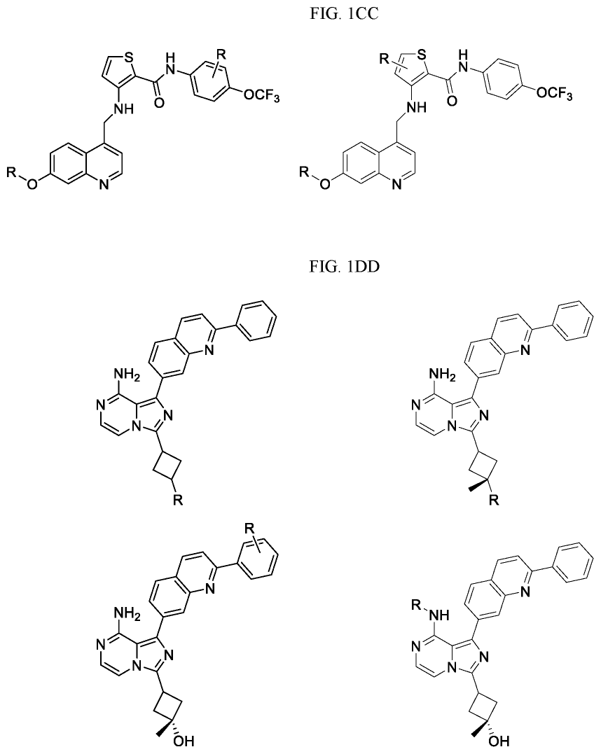

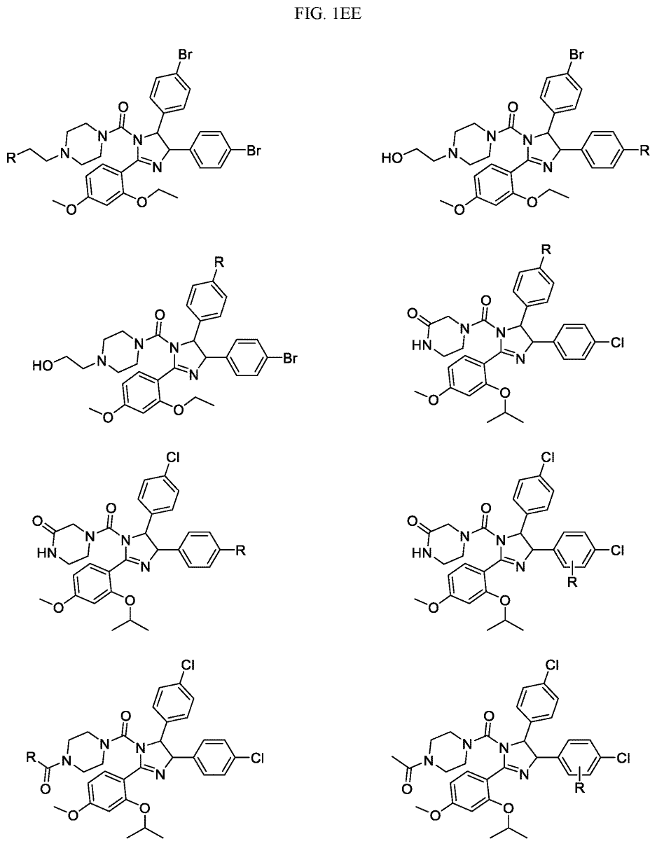

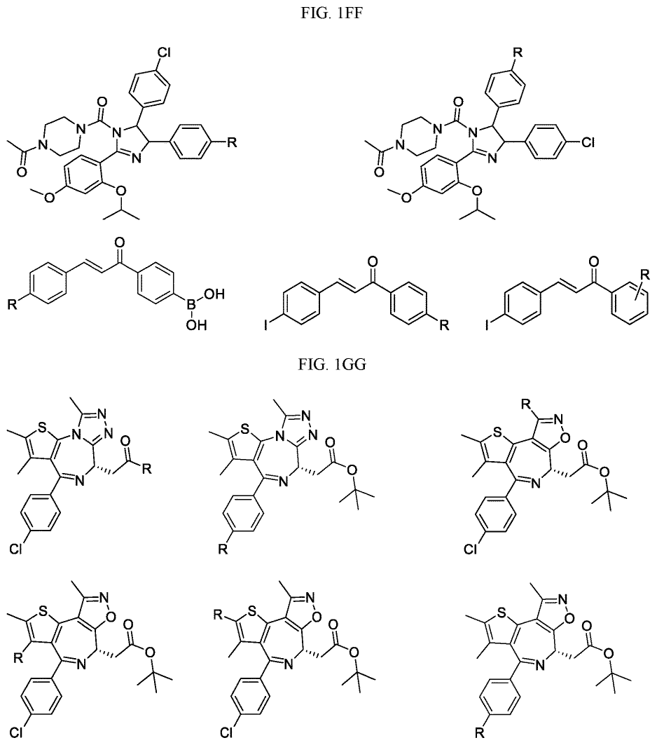

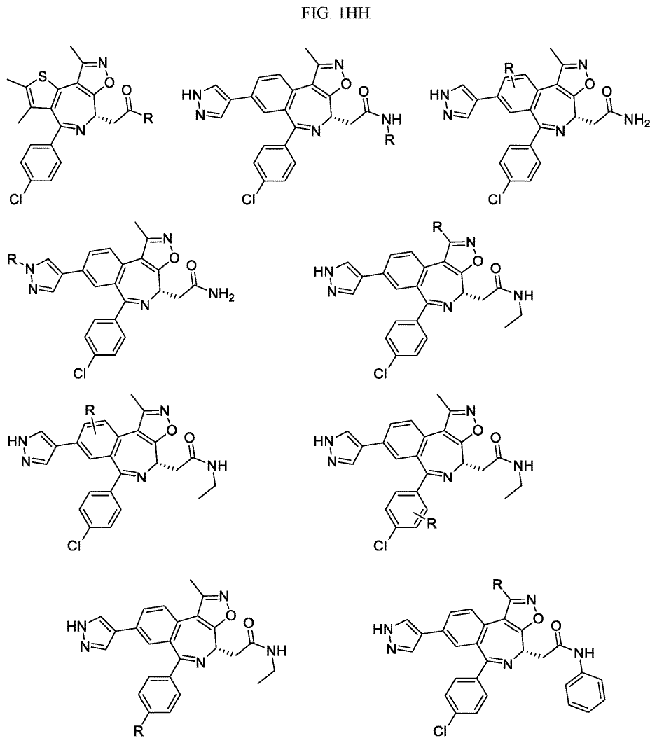

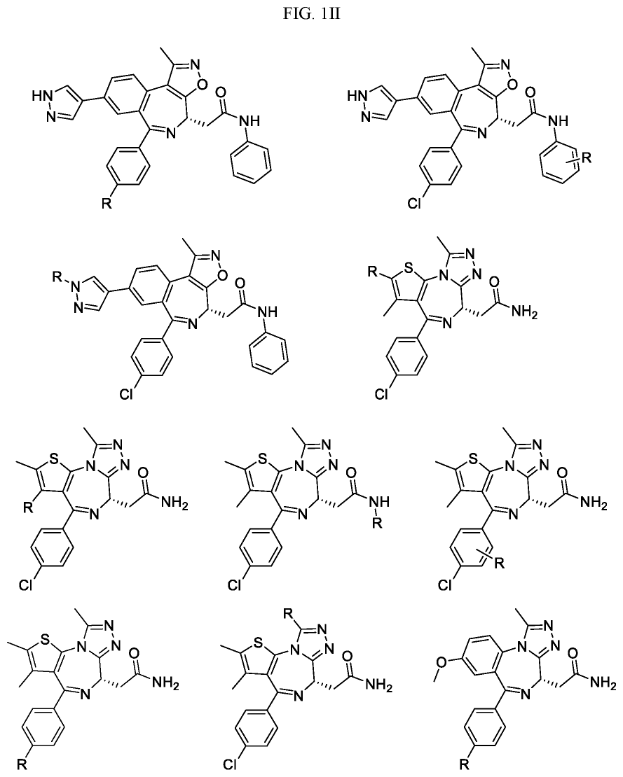

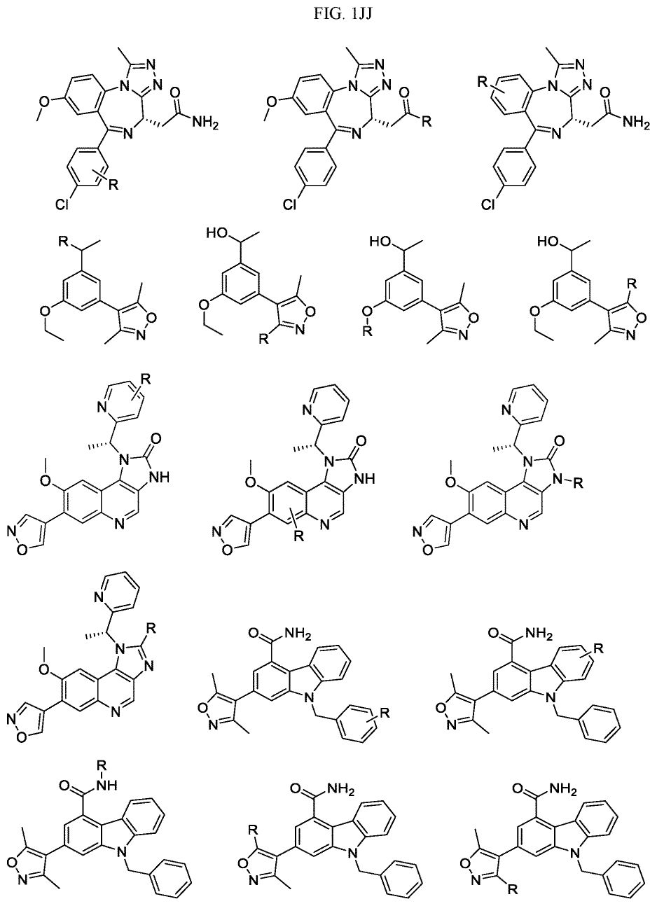

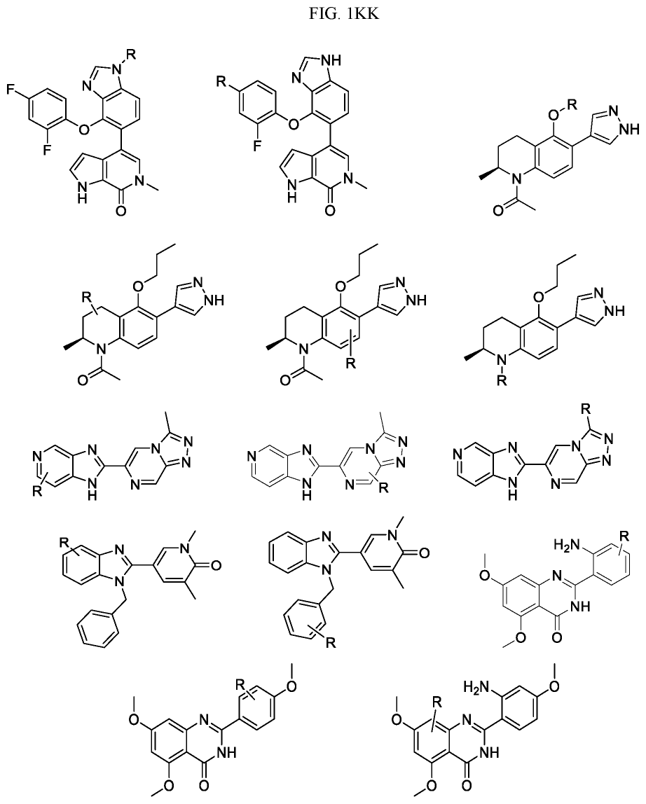

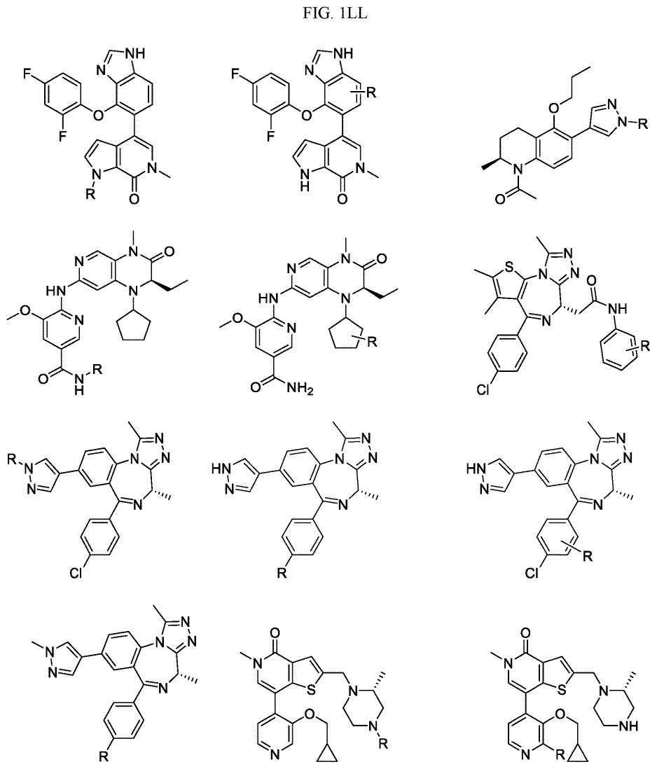

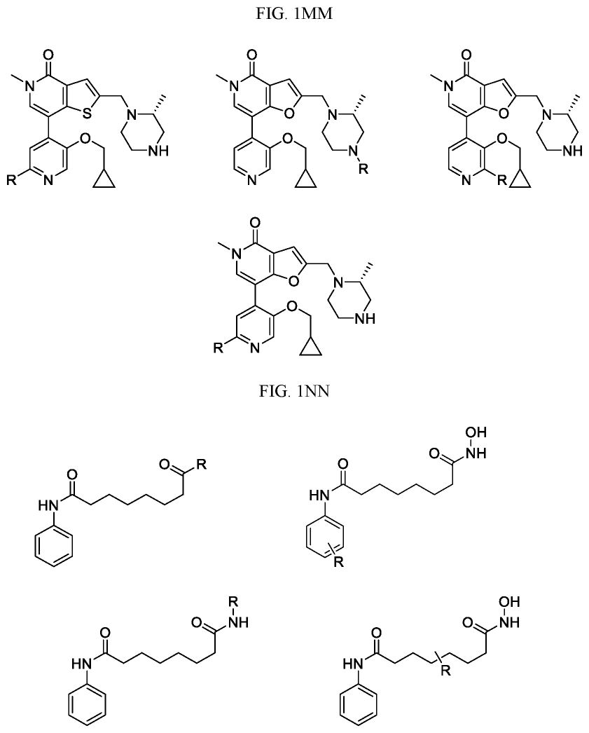

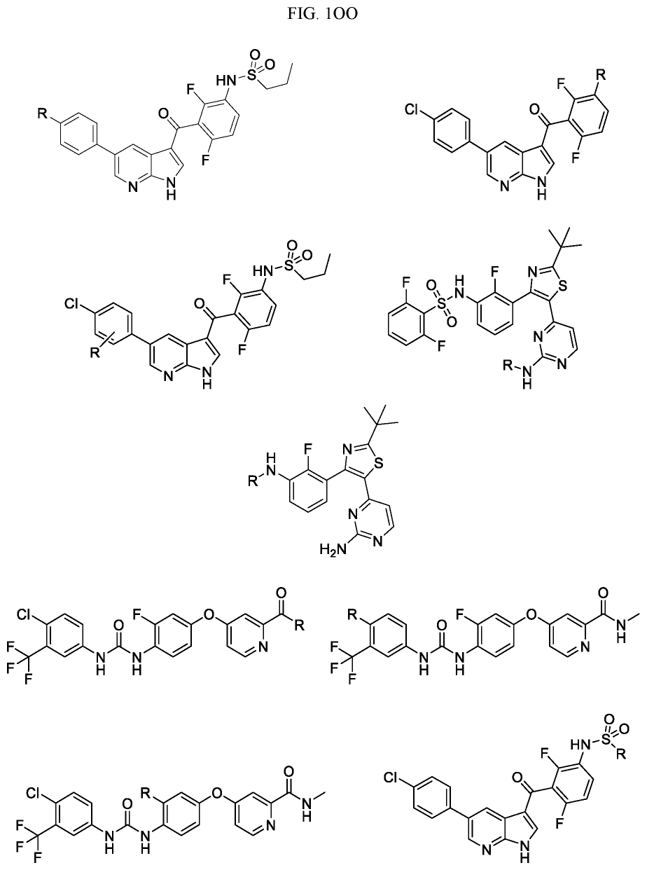

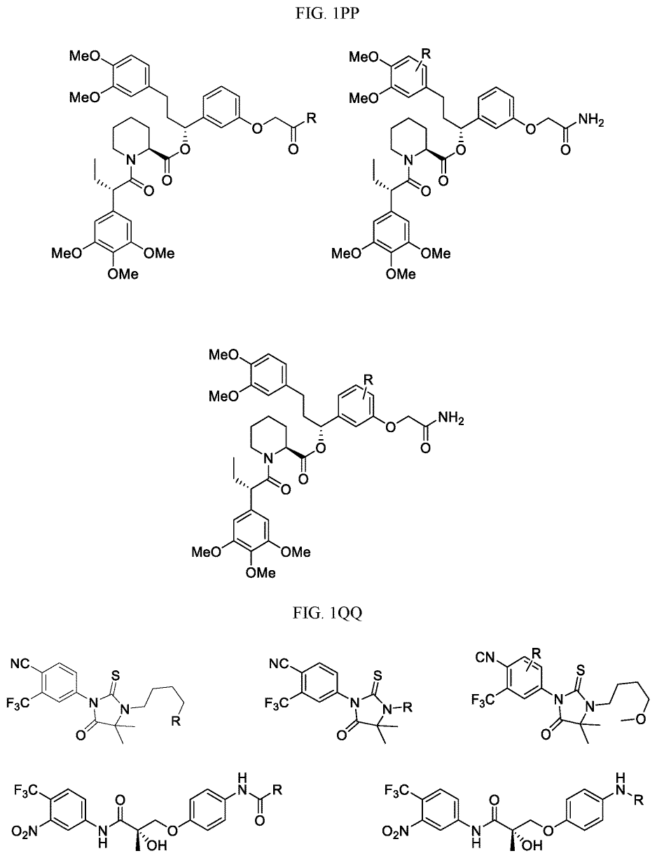

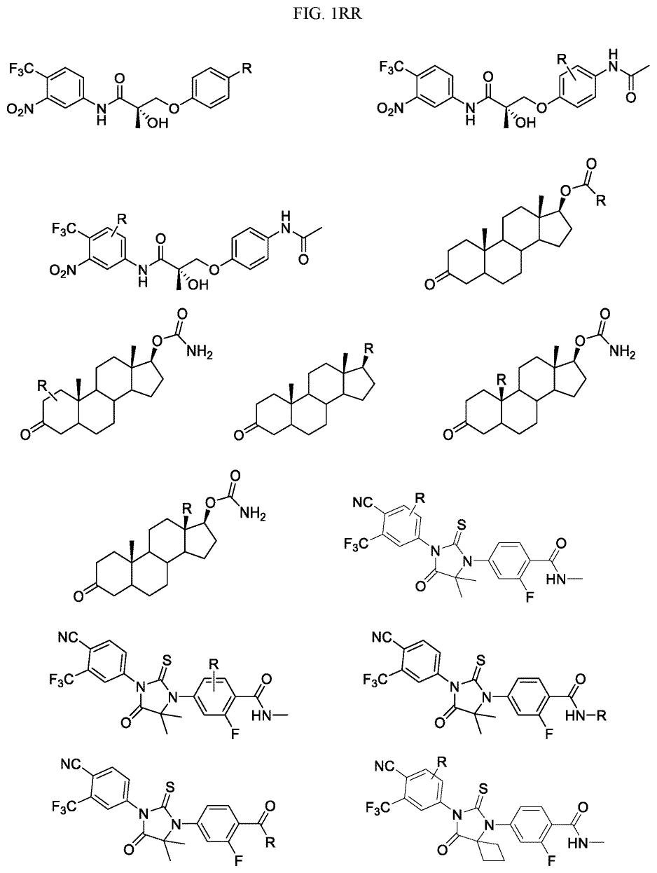

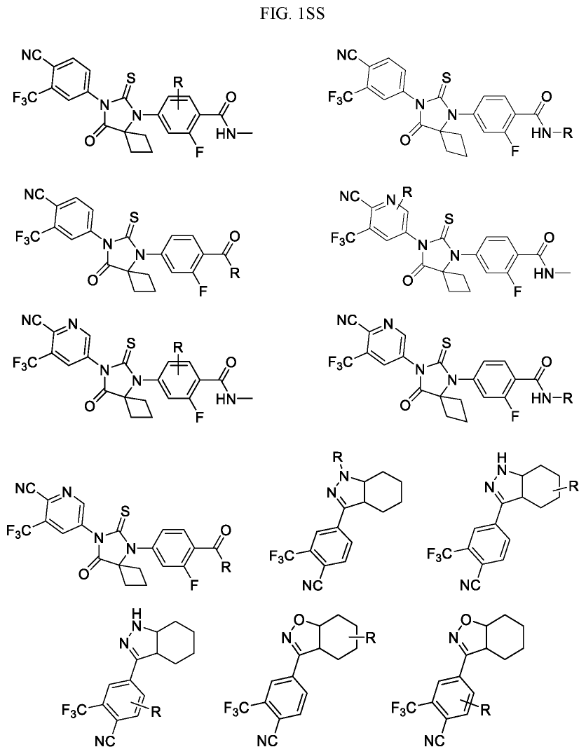

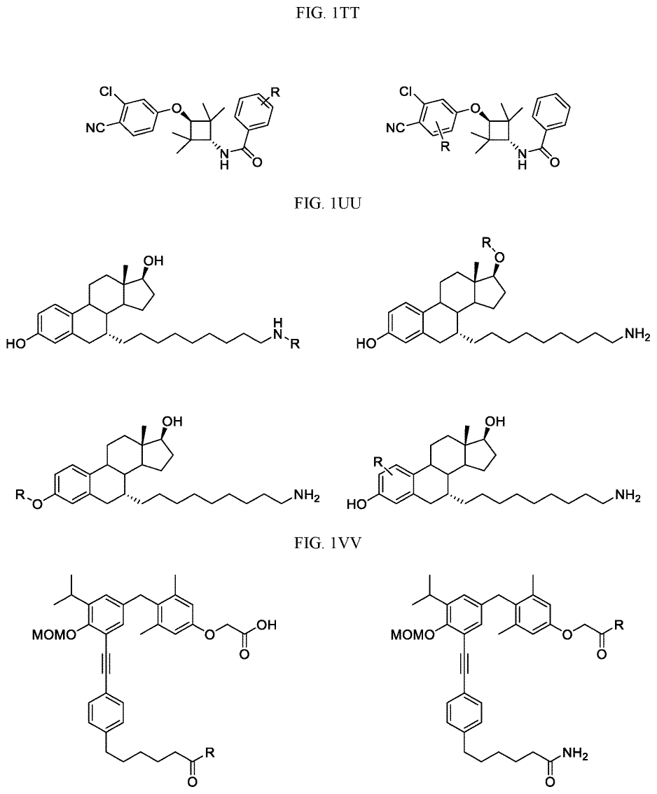

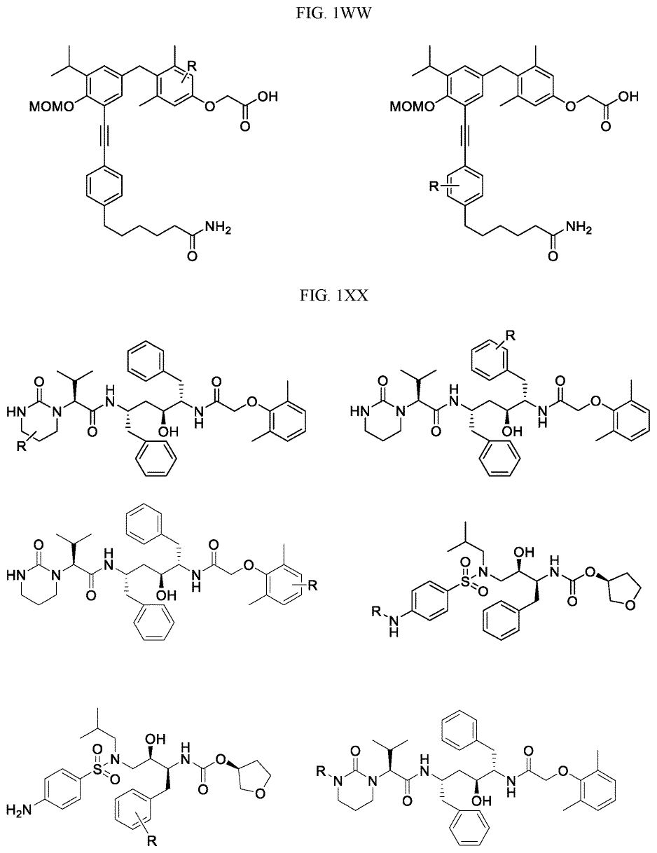

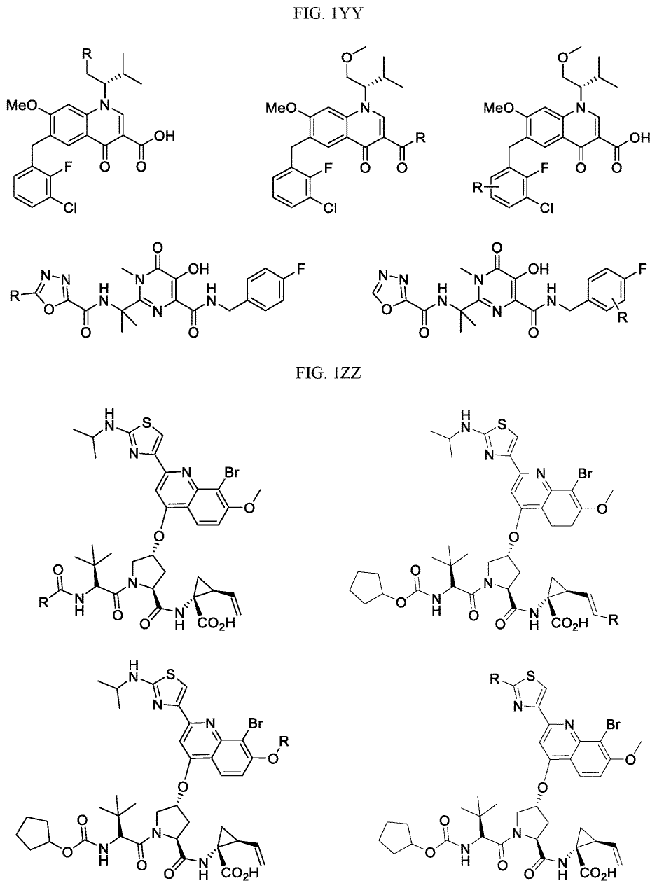

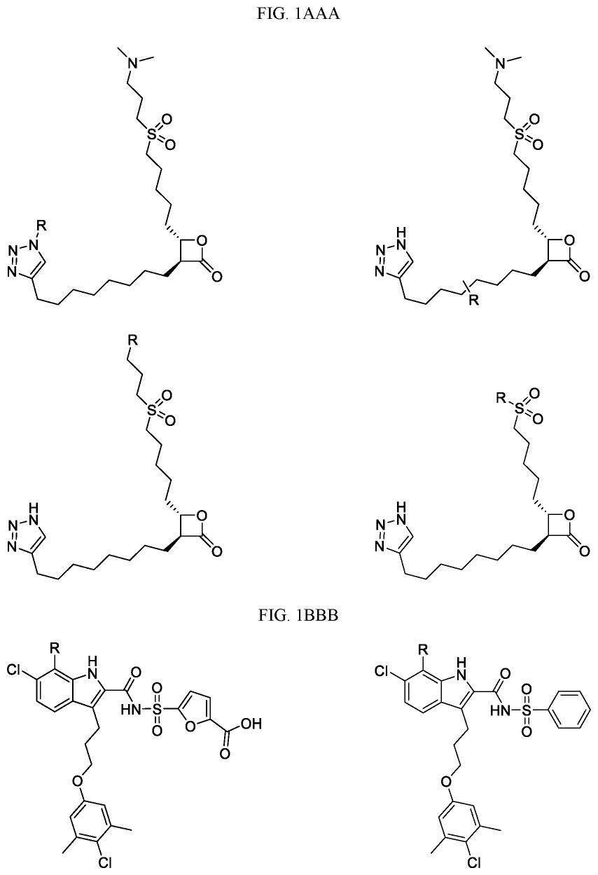

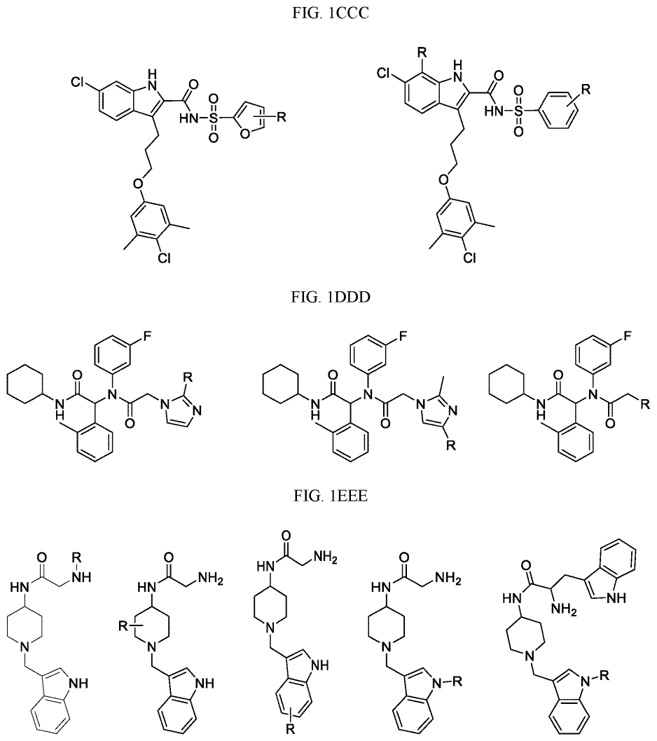

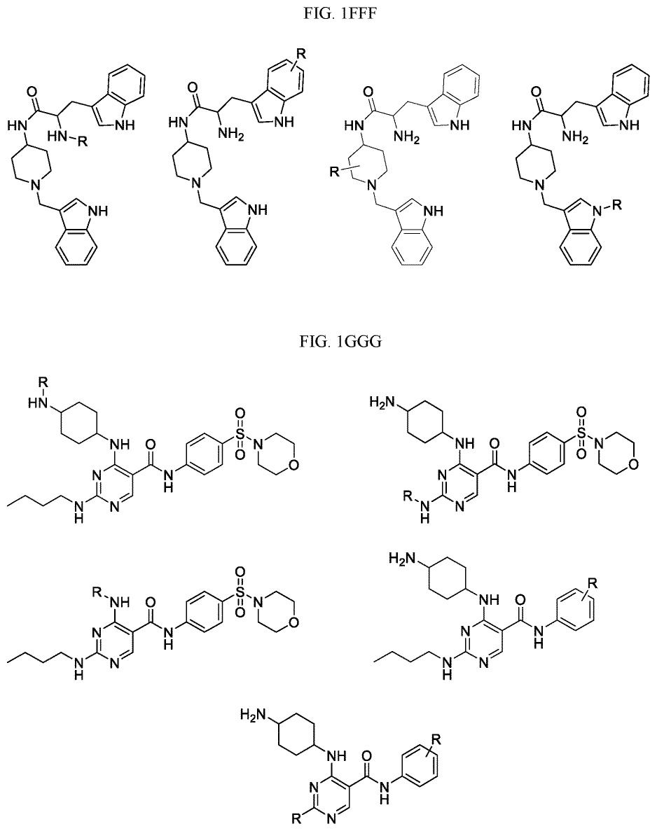

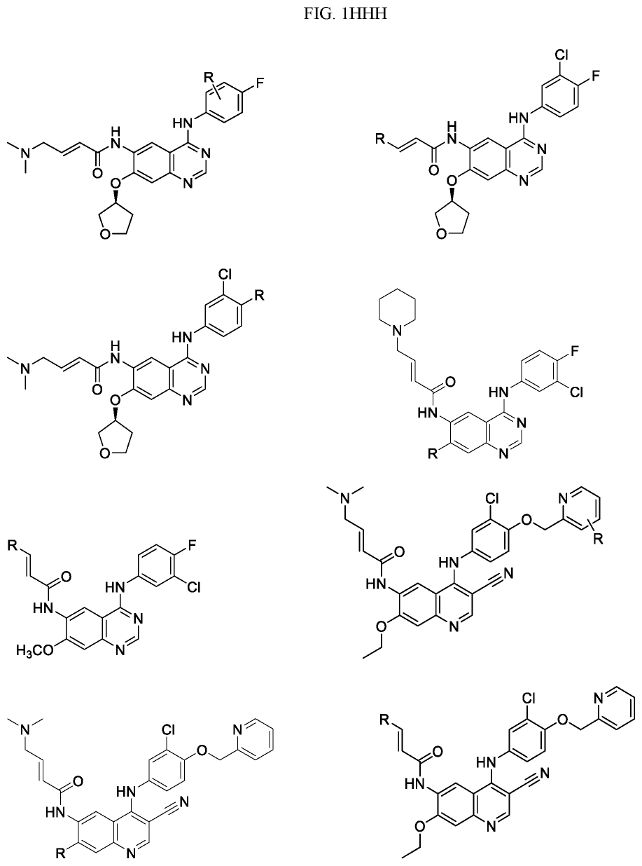

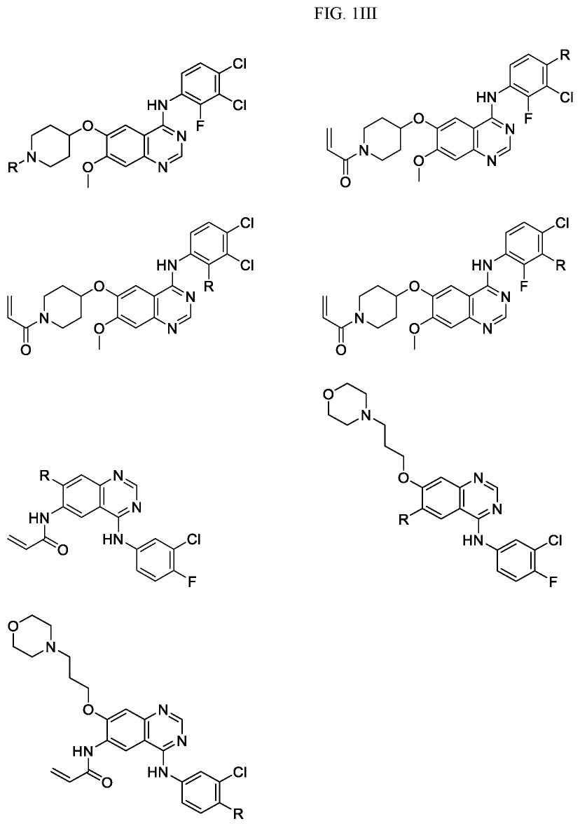

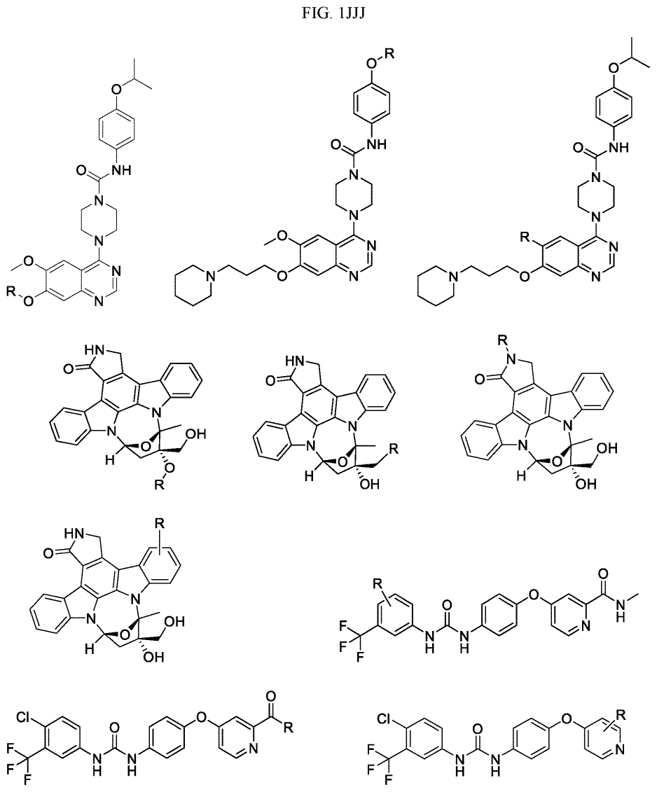

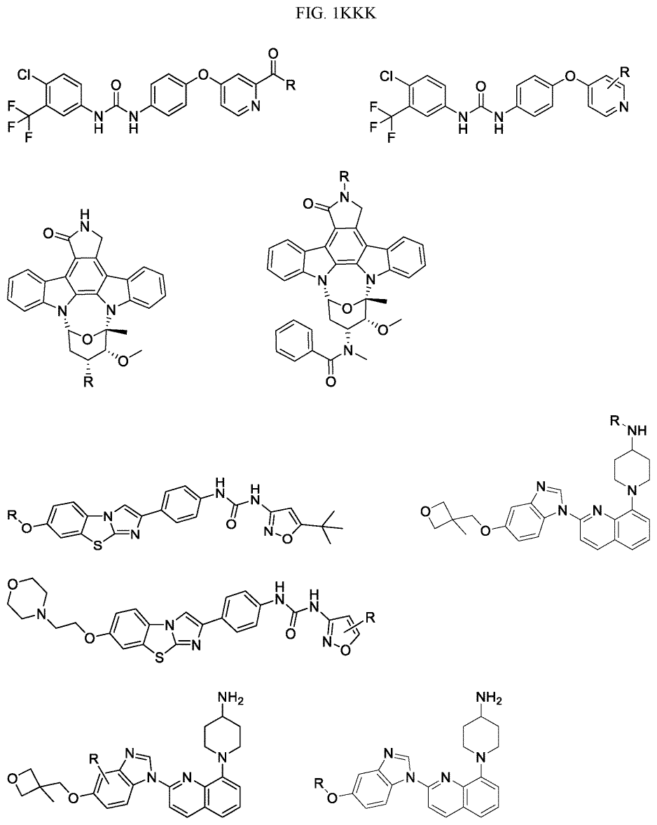

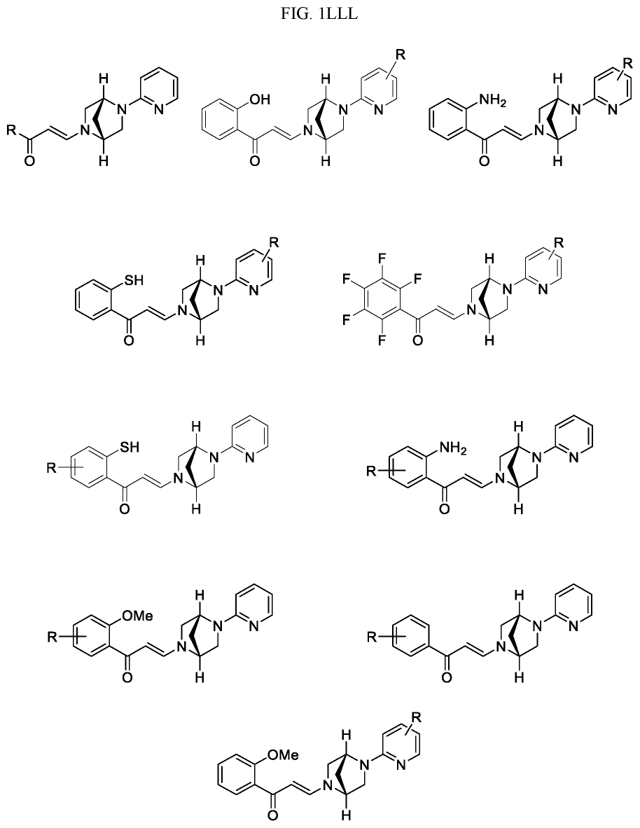

V. TARGETING LIGANDS In certain aspects, the Targeting Ligand is a ligand that covalently or non-covalently binds to a Target Protein which has been selected for proteasomal degradation by the selected Degrader. A Targeting Ligand is a molecule or moiety (for example a peptide, nucleotide, antibody, antibody fragment, aptamer, biomolecule or other chemical structure) that binds to a Target Protein, and wherein the Target Protein is a mediator of disease in a host as described in detail below. Exemplary Targeting Ligands are provided in FIGS. 1A-8PPPPP and FIGS. 10-70. Additional non-limiting examples of Targeting Ligands are provided in FIGS. 72-80. In some embodiments, the Targeting Ligand binds to an endogenous protein which has been selected for degradation as a means to achieve a therapeutic effect on the host. Illustrative Targeting Ligands include: RXR ligands, DHFR ligands, Hsp90 inhibitors, kinase inhibitors, HDM2 and MDM2 inhibitors, compounds targeting Human BET bromodomain-containing proteins, HDAC inhibitors, ligands of MerTK, ligands of IDH1, ligands of Mcl-1,ligands of SMARCA2, ligands of EGFR, ligands of RAF, ligands of cRAF, human lysine methyltransferase inhibitors, angiogenesis inhibitors, nuclear hormone receptor compounds, immunosuppressive compounds, and compounds targeting the aryl hydrocarbon receptor (AHR), among numerous others. Targeting Ligands also considered to include their pharmaceutically acceptable salts, prodrugs and isotopic derivatives.

In certain aspects, the Targeting Ligand binds to a dehalogenase enzyme in a patient or subject or in a diagnostic assay and is a haloalkane (preferably a C₁--C₁₀alkyl group which is substituted with at least one halo group, preferably a halo group at the distal end of the alkyl group (i.e., away from the Linker)). In still other embodiments, the Targeting Ligand is a haloalkyl group, wherein said alkyl group generally ranges in size from about 1 or 2 carbons to about 12 carbons in length, often about 2 to 10 carbons in length, often about 3 carbons to about 8 carbons in length, more often about 4 carbons to about 6 carbons in length. The haloalkyl groups are generally linear alkyl groups (although branched-chain alkyl groups may also be used) and are end-capped with at least one halogen group, preferably a single halogen group, often a single chloride group. Haloalkyl PT, groups for use in the present invention are preferably represented by the chemical structure -(CH₂)v-Halo where v is any integer from 2 to about 12, often about 3 to about 8, more often about 4 to about 6. Halo may be any halogen, but is preferably C1 or Br, more often C1.

In certain embodiments, the Targeting Ligand is a retinoid X receptor (RXR) agonist or antagonist. Non-limiting examples include retinol, retinoic acid, bexarotene, docosahexenoic acid, compounds disclosed in WO 9929324, the publication by Canan Koch et al. (J. Med. Chem. 1996, 39, 3229-3234) titled “Identification of the First Retinoid X Receptor Homodimer Antagonist”, WO 9712853, EP 0947496A1, WO 2016002968, and analogs thereof.

In certain embodiments, the Targeting Ligand is a DHFR agonist or antagonist. Non-limiting examples include folic acid, methotrexate, 8,10-dideazatetrahydrofolate compounds disclosed by Tian et al. (Chem. Biol. Drug Des. 2016, 87, 444-454) titled “Synthesis, Antifolate and Anticancer Activities of N5-Substituted 8,10-Dideazatetrahydrofolate Analogues”, compounds prepared by Kaur et al. (Biorg. Med. Chem. Lett. 2016, 26, 1936-1940) titled “Rational Modification of the Lead Molecule: Enhancement in the Anticancer and Dihydrofolate Reductase Inhibitory Activity”, WO 2016022890, compounds disclosed by Zhang et al. (Int. J. Antimicrob. Agents 46, 174-182) titled “New Small-Molecule Inhibitors of Dihydrofolate Reductase Inhibit Streptococcus Mutans”, modified trimethoprim analogs developed by Singh et al. (J. Med. Chem. 2012, 55, 6381-6390) titled “Mechanism Inspired Development of Rationally Designed Dihydrofolate Reductase Inhibitors as Anticancer Agents”, WO20111153310, and analogs thereof.

In certain embodiments, the Targeting Ligand derived from estrogen, an estrogen analog, SERM (selective estrogen receptor modulator), a SERD (selective estrogen receptor degrader), a complete estrogen receptor degrader, or another form of partial or complete estrogen antagonist or agonist. Examples are the partial anti-estrogens raloxifene and tamoxifen and the complete antiestrogen fulvestrant.

Non-limiting examples of anti-estrogen compounds are provided in WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In certain embodiments, the Targeting Ligand is a HSP90 inhibitor identified in Vallee et al. (J. Med. Chem. 2011, 54, 7206-7219) titled “Tricyclic Series of Heat Shock Protein 90 (Hsp90) Inhibitors Part I: Discovery of Tricyclic Imidazo[4,5-C]Pyridines as Potent Inhibitors of the Hsp90 Molecular Chaperone”, including YKB (N-[4-(3H-imidazo[4,5-C]Pyridin-2-yl)-9H-Fluoren-9-yl]-succinamide), a HSP90 inhibitors (modified) identified in Brough et al. (J Med. Chem. 2008, 51, 196-218) titled “4,5-Diarylisoxazole Hsp90 Chaperone Inhibitors: Potential Therapeutic Agents for the Treatment of Cancer”, including compound 2GJ (5-[2,4-dihydroxy-5-(1-methylethyl)phenyl]-n-ethyl-4-[4-(morpholin-4-ylmethyl)phenyl]isoxazole-3-carboxamide), the HSP90 inhibitor geldanamycin ((4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1](derivatized) or any of its derivatives (e.g. 17-alkylamino-17-desmethoxygeldanamycin (“17-AAG”) or 17-(2-dimethylaminoethyl)amino-17-desmethoxygeldanamycin (“17-DMAG”)), or a HSP90 inhibitor (modified) identified in Wright et al. (Chem. Biol. 2004, 11, 775-785) titled “Structure-Activity Relationships in Purine-Based Inhibitor Binding to Hsp90 Isoforms”, including the HSP90 inhibitor PU3.

Other non-limiting examples of Hsp90 Targeting Ligands include SNX5422 currently in phase I clinical trials Reddy et al. (Clin. LymphomaMyelomaLeuk. 2013, 13, 385-391) titled “Phase I Trial of the Hsp90 Inhibitor Pf-04929113 (Snx5422) in Adult Patients with Recurrent, Refractory Hematologic Malignancies”, or NVP-AUY922 whose anti-cancer activity was assessed by Jensen et al. (Breast Cancer Research: BCR 2008, 10, R33-R33) titled “Nvp-Auy922: A Small Molecule Hsp90 Inhibitor with Potent Antitumor Activity in Preclinical Breast Cancer Models”.

In certain embodiments, the Targeting Ligand is a kinase inhibitor identified in Millan et al. (J. Med. Chem. 2011, 54, 7797-7814) titled “Design and Synthesis of Inhaled P38 Inhibitors for the Treatment of Chronic Obstructive Pulmonary Disease”, including the kinase inhibitors Y1W and Y1X, a kinase inhibitor identified in Schenkel et al. (J. Med. Chem. 2011, 54, 8440-8450) titled “Discovery of Potent and Highly Selective Thienopyridine Janus Kinase 2 Inhibitors”, including the compounds 6TP and OTP, a kinase inhibitor identified in van Eis et al. (Biorg. Med. Chem. Lett. 2011, 21, 7367-7372) titled “2,6—Naphthyridines as Potent and Selective Inhibitors of the Novel Protein Kinase C Isozymes”, including the kinase inhibitors 07U and YCF identified in Lountos et al. (J. Struct. Biol. 2011, 176, 292-301) titled “Structural Characterization of Inhibitor Complexes with Checkpoint Kinase 2 (Chk2), a Drug Target for Cancer Therapy”, including the kinase inhibitors XK9 and NXP, afatinib, fostamatinib, gefitinib, lenvatinib, vandetanib, Gleevec, pazopanib, AT-9283, TAE684, nilotanib, NVP-BSK805, crizotinib, JNJ FMS, foretinib, OSI-027, OSI-930, or OSI-906.

In certain embodiments, the Targeting Ligand is a HDM2/MDM2 inhibitor identified in Vassilev et al. (Science 2004, 303, 844-848) titled “In Vivo Activation of the P53 Pathway by Small-Molecule Antagonists of Mdm2”, and Schneekloth et al. (Bioorg. Med. Chem. Lett. 2008, 18, 5904-5908) titled “Targeted Intracellular Protein Degradation Induced by a Small Molecule: En Route to Chemical Proteomics”, including the compounds nutlin-3, nutlin-2, and nutlin-1.

In certain embodiments, the Targeting Ligand is a Human BET Bromodomain Targeting Ligand identified in Filippakopoulos et al. (Nature 2010, 468, 1067-1073) titled “Selective Inhibition of Bet Bromodomains” such as JQ1; a ligand identified in Nicodeme et al. (Nature 2010, 468, 1119-1123) titled “Suppression of Inflammation by a Synthetic Histone Mimic”; Chung et al. (J Med. Chem. 2011, 54, 3827-3838) titled “Discovery and Characterization of Small Molecule Inhibitors of the Bet Family Bromodomains”; a compound disclosed in Hewings et al. (J. Med. Chem. 2011, 54, 6761-6770) titled “3,5-Dimethylisoxazoles Act as Acetyl-Lysine-Mimetic Bromodomain Ligands”; a ligand identified in Dawson et al. (Nature 2011, 478, 529-533) titled “Inhibition of Bet Recruitment to Chromatin as an Effective Treatment for MLL-Fusion Leukaemia”; or a ligand identified in the following patent applications US 2015/0256700, US 2015/0148342, WO 2015/074064, WO 2015/067770, WO 2015/022332, WO 2015/015318, and WO 2015/011084.

In certain embodiments, the Targeting Ligand is a HDAC Targeting Ligand identified in Finnin et al. (Nature 1999, 401, 188-193) titled “Structures of a Histone Deacetylase Homologue Bound to the Tsa and Saha Inhibitors”, or a ligand identified as Formula (I) in PCT WO0222577.

In certain embodiments, the Targeting Ligand is a Human Lysine Methyltransferase ligand identified in Chang et al. (Nat StructMolBiol 2009, 16, 312-317) titled “Structural Basis for G9a-Like Protein Lysine Methyltransferase Inhibition by Bix-01294”, a ligand identified in Liu et al. (J Med Chem 2009, 52, 7950-7953) titled “Discovery of a 2,4-Diamino-7-Aminoalkoxyquinazoline as a Potent and Selective Inhibitor of Histone Lysine Methyltransferase G9a”, azacitidine, decitabine, or an analog thereof.

In certain embodiments, the Targeting Ligand is an angiogenesis inhibitor. Non-limiting examples of angiogenesis inhibitors include GA-1, estradiol, testosterone, ovalicin, fumagillin, and analogs thereof.

In certain embodiments, the Targeting Ligand is an immunosuppressive compound.

Non-limiting examples of immunosuppressive compounds include: AP21998, hydrocortisone, prednisone, prednisolone, methylprednisolone, beclometasone dipropionate, methotrexate, ciclosporin, tacrolimus, actinomycin, and analogues thereof.

In certain embodiments, the Targeting Ligand is an Aryl Hydrocarbon Receptor (AHR) ligand. Non-limiting examples of AHR ligands include: apigenin, SRi, LGC006, and analogues thereof.

In certain embodiments, the Targeting Ligand is a MerTK or Mer Targeting ligand.

Non-limiting examples of MerTK Targeting Ligands are included in WO2013/177168 and WO2014/085225, both titled “Pyrimidine Compounds for the Treatment of Cancer” filed by Wang, et al.

In certain embodiments, the Targeting Ligand is an EGFR ligand. In certain embodiments the Targeting Ligand is an EGRF ligand selected from Afatinib, Dacomitinib, Neratinib, Poziotinib, and Canertinib, or derivatives thereof.

In certain embodiments, the Targeting Ligand is a FLT3 Ligand. In certain embodiments, the Targeting Ligand is a FLT3 ligand selected from Tandutinib, Lestaurtinib, Sorafenib, Midostaurin, Quizartinib, and Crenolanib.

In certain embodiments, the Targeting Ligand is a RAF inhibitor. In certain embodiments the Targeting Ligand is a RAF inhibitor selected from Dabrafenib, Regorafenib, and Vemurafenib. In certain embodiments the Targeting Ligand is a cRAF inhibitor.

In some embodiments, the Targeting Ligand is an Ubc9 SUMO E2 ligase 5F6D Targeting Ligand including but not limited to those described in “Insights into the Allosteric Inhibition of the SUMO E2 Enzyme Ubc9.” Hewitt, W. M., et. al. (2016) Angew.Chem.Int.Ed.Engl. 55: 5703-5707.

In another embodiment, the Targeting Ligand is a Tank1 Targeting Ligand including but not limited to those described in “Structure of human tankyrase 1 in complex with small-molecule inhibitors PJ34 and XAV939.” Kirby, C.A., Cheung, A., Fazal, A., Shultz, M.D., Stams, T, (2012) Acta Crystallogr., Sect. F 68: 115-118; and “Structure-Efficiency Relationship of [1,2,4]Triazol-3-ylamines as Novel Nicotinamide Isosteres that Inhibit Tankyrases.” Shultz, M.D., et al. (2013) J. Med. Chem. 56: 7049-7059.

In another embodiment, the Targeting Ligand is a SH2 domain of pp60 Src Targeting Ligand including but not limited to those described in “Requirements for Specific Binding of Low Affinity Inhibitor Fragments to the SH2 Domain of pp60Src Are Identical to Those for High Affinity Binding of Full Length Inhibitors,” Gudrun Lange, et al., J. Med. Chem. 2003, 46, 5184-5195.

In another embodiment, the Targeting Ligand is a Sec7 domain Targeting Ligand including but not limited to those described in “The Lysosomal Protein Saposin B Binds Chloroquine,” Huta, B. P., et al., (2016) Chemmedchem 11: 277.

In another embodiment, the Targeting Ligand is a Saposin-B Targeting Ligand including but not limited to those described in “The structure of cytomegalovirus immune modulator UL141 highlights structural Ig-fold versatility for receptor binding” I. Nemcovicova and D. M. Zajonc Acta Cryst. (2014). D70, 851-862.

In another embodiment, the Targeting Ligand is a Protein 5100-A7 20WS Targeting Ligand including but not limited to those described in “2WOS STRUCTURE OF HUMAN S100A7 IN COMPLEX WITH 2,6 ANS” DOI: 10.2210/pdb2wos/pdb; and “Identification and Characterization of Binding Sites on S100A7, a Participant in Cancer and Inflammation Pathways.” Leon, R., Murray, et al., (2009) Biochemistry 48: 10591-10600.

In another embodiment, the Targeting Ligand is a Phospholipase A2 Targeting Ligand including but not limited to those described in “Structure-based design of the first potent and selective inhibitor of human non-pancreatic secretory phospholipase A2” Schevitz, R. W., et al., Nat. Struct. Biol. 1995, 2, 458-465.

In another embodiment, the Targeting Ligand is a PHIP Targeting Ligand including but not limited to those described in “A Poised Fragment Library Enables Rapid Synthetic Expansion Yielding the First Reported Inhibitors of PHIP(2), an Atypical Bromodomain” Krojer, T.; et al. Chem. Sci. 2016, 7, 2322-2330.

In another embodiment, the Targeting Ligand is a PDZ Targeting Ligand including but not limited to those described in “Discovery of Low-Molecular-Weight Ligands for the AF6 PDZ Domain” Mangesh Joshi, et al. Angew. Chem. Int. Ed. 2006, 45, 3790-3795.

In another embodiment, the Targeting Ligand is a PARP15 Targeting Ligand including but not limited to those described in “Structural Basis for Lack of ADP-ribosyltransferase Activity in Poly(ADP-ribose) Polymerase-13/Zinc Finger Antiviral Protein.” Karlberg, T., et al., (2015) J.Biol.Chem. 290: 7336-7344.

In another embodiment, the Targeting Ligand is a PARP14 Targeting Ligand including but not limited to those described in “Discovery of Ligands for ADP-Ribosyltransferases via Docking-Based Virtual Screening” Andersson, C. D., et al. (2012) J. Med. Chem. 55: 7706-7718; “Family-wide chemical profiling and structural analysis of PARP and tankyrase inhibitors” Wahlberg, E., et al. (2012) Nat.Biotechnol. 30: 283-288.; “Discovery of Ligands for ADP-Ribosyltransferases via Docking-Based Virtual Screening” Andersson, C. D., et al. (2012) J. Med. Chem. 55: 7706-7718.

In another embodiment, the Targeting Ligand is a MTH1 Targeting Ligand including but not limited to those described in “MTH1 inhibition eradicates cancer by preventing 5 sanitation of the dNTP pool” Helge Gad, et. al. Nature, 2014, 508, 215-221.

In another embodiment, the Targeting Ligand is a mPGES-1 Targeting Ligand including but not limited to those described in “Crystal Structures of mPGES-1 Inhibitor Complexes Form a Basis for the Rational Design of Potent Analgesic and Anti-Inflammatory Therapeutics.” Luz, J. G., et al., (2015) J. Med. Chem. 58: 4727-4737.

In another embodiment, the Targeting Ligand is a FLAP- 5-lipoxygenase-activating protein Targeting Ligand including but not limited to those described in “Crystal structure of inhibitor-bound human 5-lipoxygenase-activating protein” Ferguson, A.D., McKeever, B.M., Xu, S., Wisniewski, D., Miller, D.K., Yamin, T.T., Spencer, R.H., Chu, L., Ujjainwalla, F., Cunningham, B.R., Evans, J.F., Becker, J.W. (2007) Science 317: 510-512.

In another embodiment, the Targeting Ligand is a FA Binding Protein Targeting Ligand including but not limited to those described in “A Real-World Perspective on Molecular Design.” Kuhn, B.; et al. J. Med. Chem. 2016, 59, 4087-4102.

In another embodiment, the Targeting Ligand is a BCL2 Targeting Ligand including but not limited to those described in “ABT-199, a potent and selective BCL-2 inhibitor, 20 achieves antitumor activity while sparing platelets.” Souers, A. J., et al. (2013) NAT.MED. (N.Y.) 19: 202-208.

In another embodiment, the Targeting Ligand is a NF2L2 Targeting Ligand.

In another embodiment, the Targeting Ligand is a CTNNB1 Targeting Ligand.

In another embodiment, the Targeting Ligand is a CBLB Targeting Ligand.

In another embodiment, the Targeting Ligand is a BCL6 Targeting Ligand.

In another embodiment, the Targeting Ligand is a RASK Targeting Ligand.

In another embodiment, the Targeting Ligand is a TNIK Targeting Ligand.

In another embodiment, the Targeting Ligand is a MEN1 Targeting Ligand.

In another embodiment, the Targeting Ligand is a PI3Ka Targeting Ligand.

In another embodiment, the Targeting Ligand is a IDO1 Targeting Ligand.

In another embodiment, the Targeting Ligand is a MCL1 Targeting Ligand.

In another embodiment, the Targeting Ligand is a PTPN2 Targeting Ligand.

In another embodiment, the Targeting Ligand is a HER2 Targeting Ligand. In another embodiment, the Targeting Ligand is an EGFR Targeting Ligand. In one embodiment the Targeting Ligand is selected from erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer). The linker can be placed on these Targeting Ligands in any location that does not interfere with the Ligands binding to EGFR.

Non-limiting examples of Linker binding locations are provided in the below tables. In one embodiment, the EGFR Targeting Ligand binds the L858R mutant of EGFR. In another embodiment, the EGFR Targeting Ligand binds the T790M mutant of EGFR. In another embodiment, the EGFR Targeting Ligand binds the C797G or C797S mutant of EGFR. In one embodiment, the EGFR Targeting Ligand is selected from erlotinib, gefitinib, afatinib, neratinib, and dacomitinib and binds the L858R mutant of EGFR. In another embodiment, the EGFR Targeting Ligand is selected from osimertinib, rociletinib, olmutinib, naquotinib, nazartinib, PF-06747775, Icotinib, Neratinib, Avitinib, Tarloxotinib, PF-0645998, Tesevatinib, Transtinib, WZ-3146, WZ8040, and CNX-2006 and binds the T790M mutant of EGFR. In another embodiment, the EGFR Targeting Ligand is EAI045 and binds the C797G or C797S mutant of EGFR.

In one embodiment, the protein target and Targeting Ligand pair are chosen by screening a library of ligands. Such a screening is exemplified in “Kinase Inhibitor Profiling Reveals Unexpected Opportunities to Inhibit Disease-Associated Mutant Kinases” by Duong-Ly et al.; Cell Reports 14, 772-781 Feb. 2, 2016.

In certain aspects the targeting ligand is a peptide, polypeptide, or protein. A protein or peptide must contain at least two amino acids, and the maximum number of amino acids present within the protein or peptide's sequence is typically comparable to up to that found in nature.

Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. In certain aspects the terms the terms “peptide,” “polypeptide,” and “protein” are used interchangeably, and refer to a compound comprised of amino acid residues covalently linked by peptide bonds.

In one embodiment, the protein target and Targeting Ligand pair are discovered by 5 screening promiscuous kinase binding ligands for context-specific degradation. Non-limiting examples of targeting ligands are shown below and are found in “Optimized Chemical Proteomics Assay for Kinase Inhibitor Profiling” Guillaume Medard, Fiona Pachl, Benjamin Ruprecht, Susan Klaeger, Stephanie Heinzlmeir, Dominic Helm, Huichao Qiao, Xin Ku, Mathias Wilhelm, Thomas Kuehne, Zhixiang Wu, Antje Dittmann, Carsten Hopf, Karl Kramer, 10 and Bernhard Kuster J. Proteome Res., 2015, 14(3), pp 1574-1586:

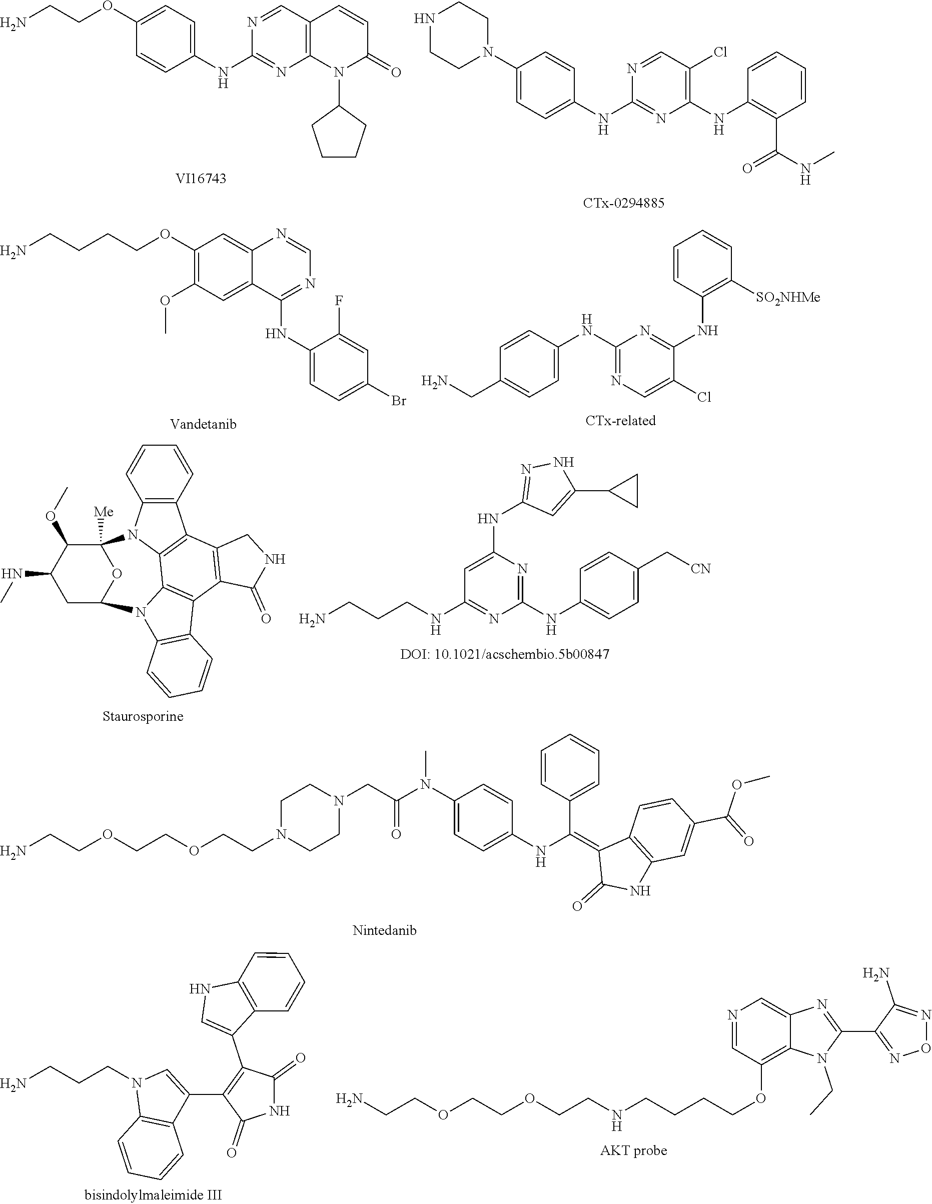

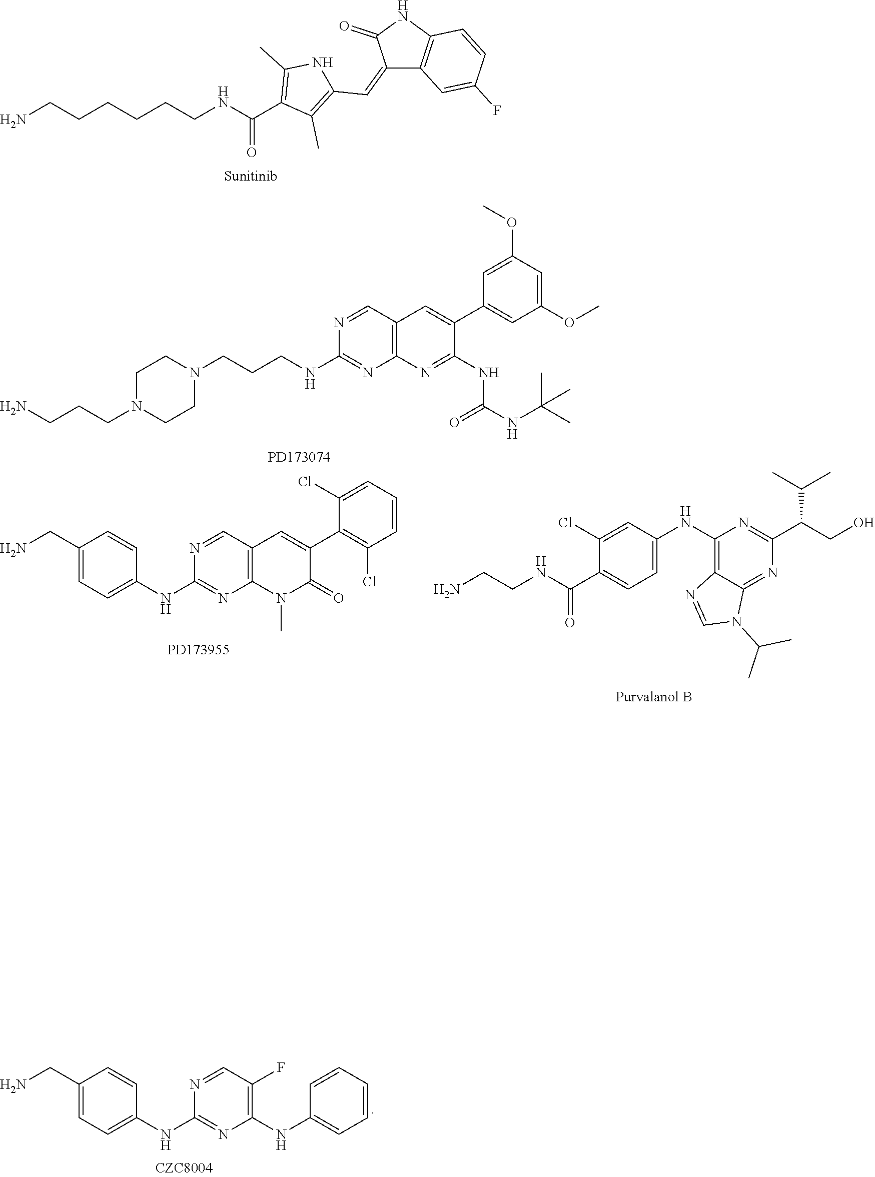 [SMILES: Cc1[nH]c(/C=C2\C(=O)Nc3ccc(F)cc32)c(C)c1C(=O)NCCCCCCN ; COc1cc(OC)cc(-c2cc3cnc(NCCCN4CCN(CCCN)CC4)nc3nc2NC(=O)NC(C)(C)C)c1 ; Cn1c(=O)c(-c2c(Cl)cccc2Cl)cc2cnc(Nc3ccc(CN)cc3)nc21 ; CC(C)[C@H](CO)c1nc(Nc2ccc(C(=O)NCCN)c(Cl)c2)c2ncn(C(C)C)c2n1 ; NCc1ccc(Nc2ncc(F)c(Nc3ccccc3)n2)cc1]

These ligands can be attached to linkers as shown below:

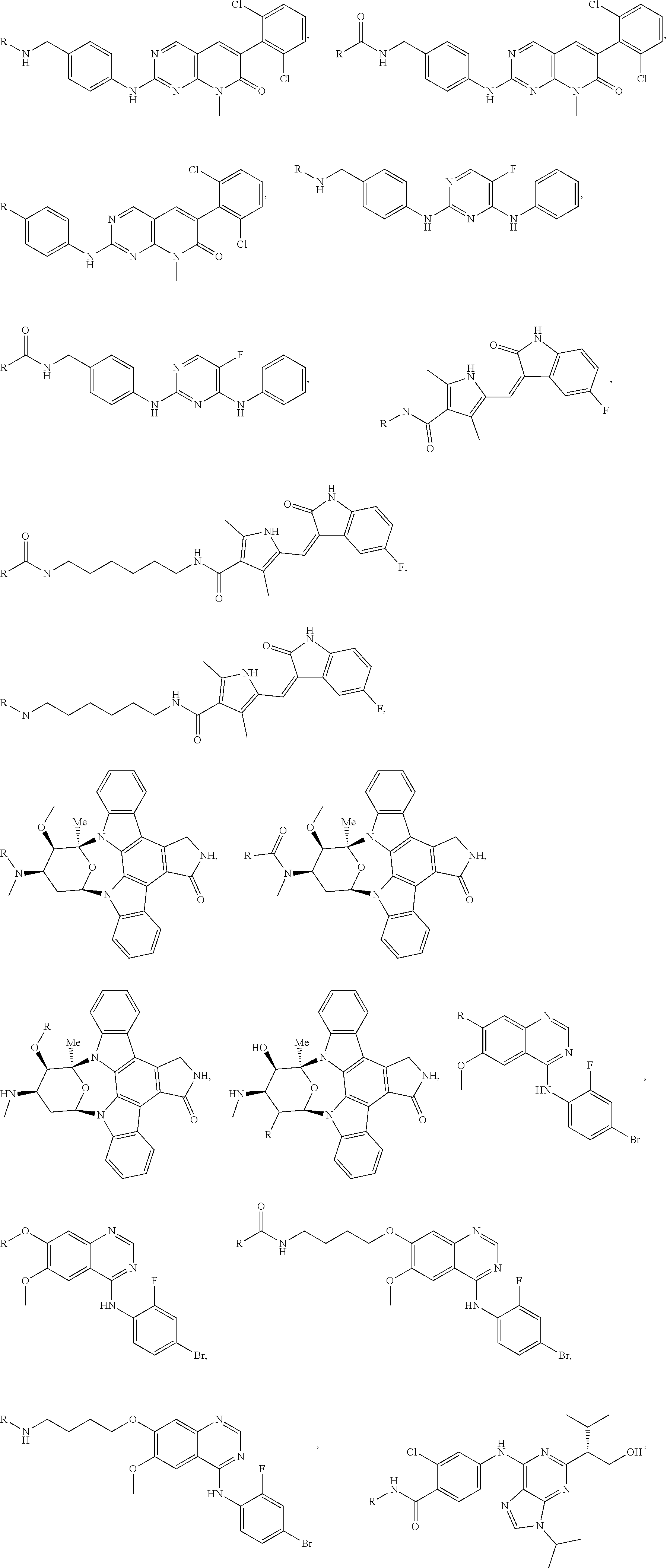

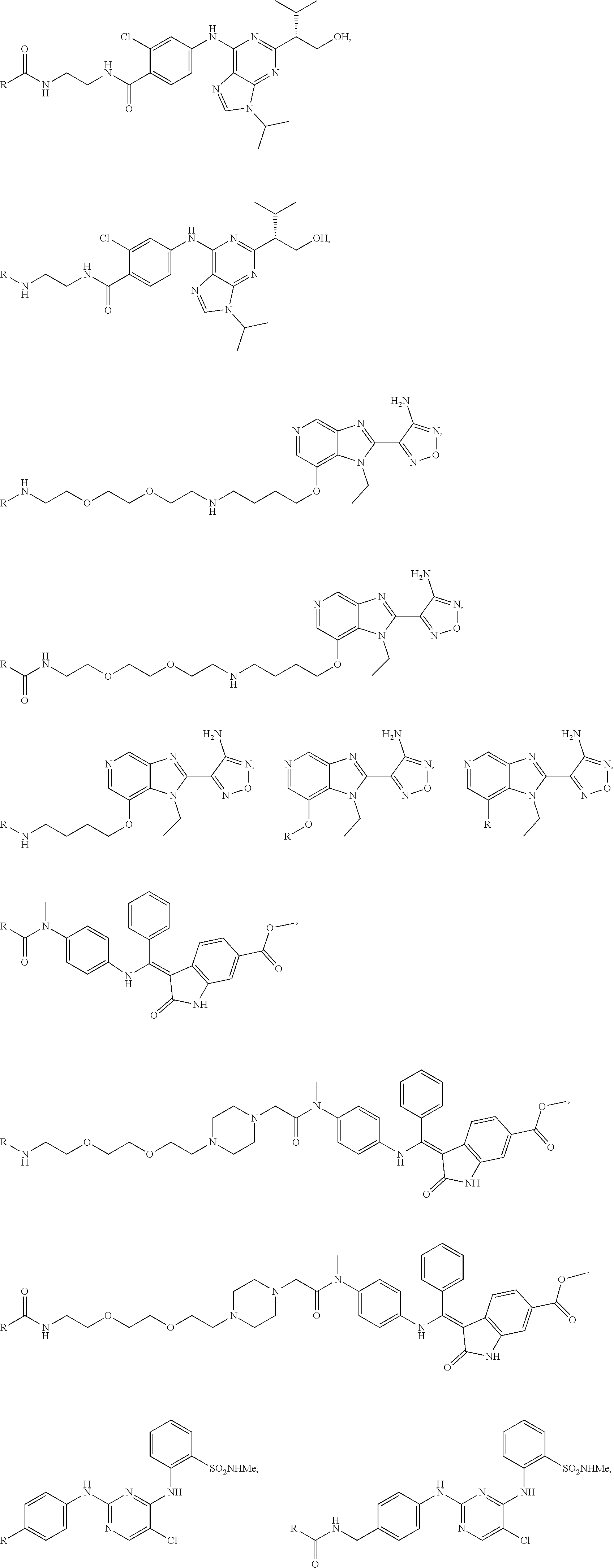

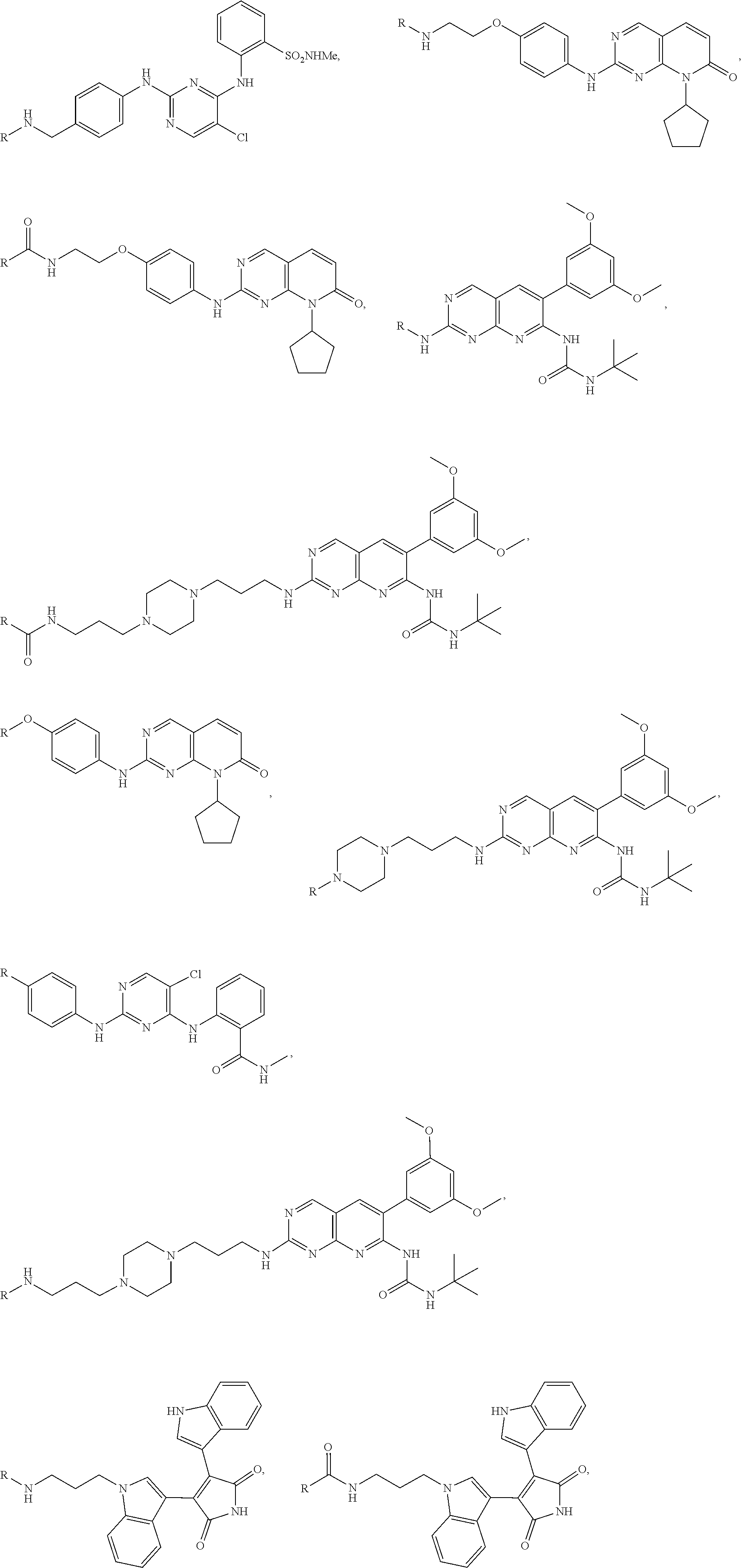

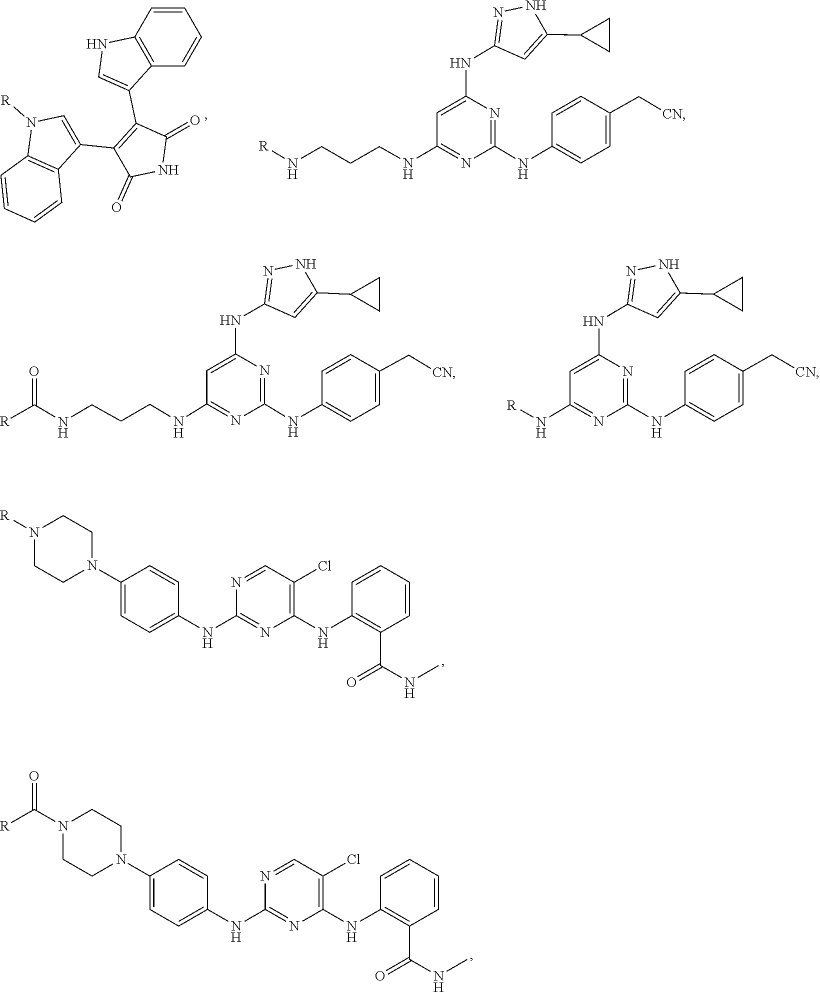

R is the point at which the Linker is attached.

In another embodiment, the Targeting Ligand is selected from a DOTL1-Ligand, a CBP Ligand, an ERK1 Ligand, an ERK2 Ligand, a JAK2 Ligand, an FGFR3 Ligand, an FGFR4 Ligand, a WDR5 Ligand, a PAK4 Ligand, a BRAF Ligand, a KRAS Ligand, a BTK Ligand, and a SHOC2 Ligand. In another embodiment, the Targeting Ligand is selected from a UCHL1 Ligand, a USP1 Ligand, a USP2 Ligand, a USP4 Ligand, a USP6 Ligand, a USP7 Ligand, a USP8 Ligand, a USP9x Ligand, a USP10 Ligand, a USP 11 Ligand, a USP13 Ligand, a USP14 Ligand, a USP17 Ligand, and a USP28 Ligand.

In certain aspects the Targeting Ligand is a CSF1R Targeting Ligand.

In certain aspects the Targeting Ligand is a DGK Targeting Ligand.

In certain aspects the Targeting Ligand is a HPK1Targeting Ligand.

In certain aspects the Targeting Ligand is a BCL6Targeting Ligand.

In certain aspects the Targeting Ligand is a SOS1 Targeting Ligand.

In certain aspects the Targeting Ligand is a TYK2 Targeting Ligand.

In certain aspects the Targeting Ligand is a USP1 Targeting Ligand.

In certain aspects the Targeting Ligand is a Co-REST Targeting Ligand. In certain embodiments the Targeting Ligand is a TYK2 Targeting Ligand of Formula:

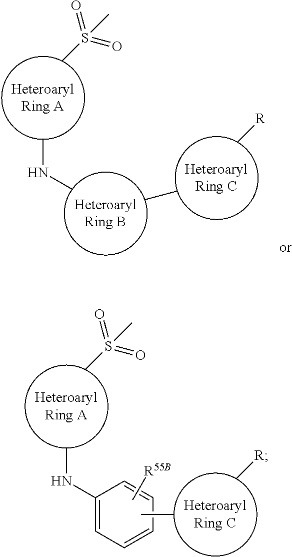

wherein: R is the attachment point where Linker is attached; Heteroaryl Ring A is a heteroaryl group optionally substituted with 1, 2, or 3 substituents selected from R^{55A}; Heteroaryl Ring B is a heteroaryl group optionally substituted with 1, 2, or 3 substituents selected from R⁵⁵B.

Heteroaryl Ring C is a heteroaryl group optionally substituted with 1, 2, or 3 10 substituents selected from R^{55C}; and RssA, R⁵⁵B, and Rssc are independently selected at each instance from alkyl, haloalkyl, halogen, cyano, —NR⁷R⁸, and —OR⁷.

In certain embodiments Heteroaryl Ring A is pyridine.

In certain embodiments Heteroaryl Ring B is pyridine.

In certain embodiments Heteroaryl Ring C is pyridine.

In certain embodiments the Targeting Ligand is a SOS1 Targeting Ligand of Formula:

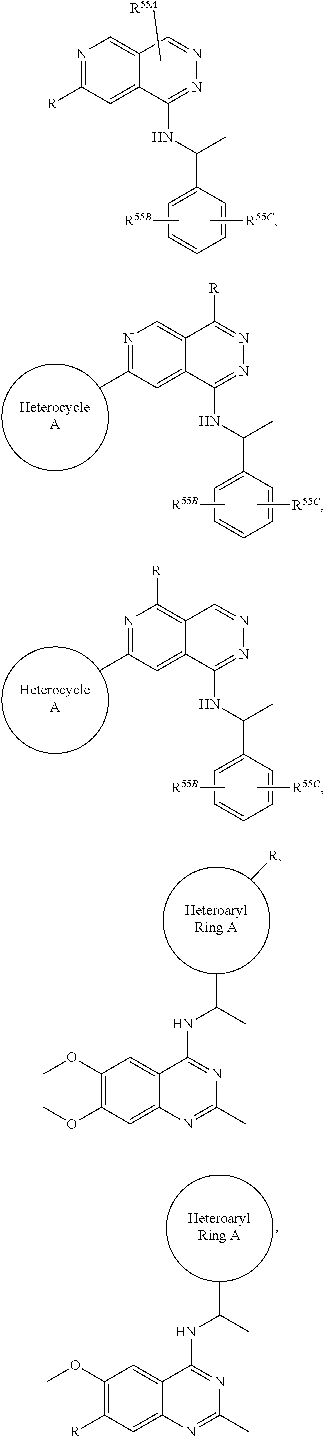

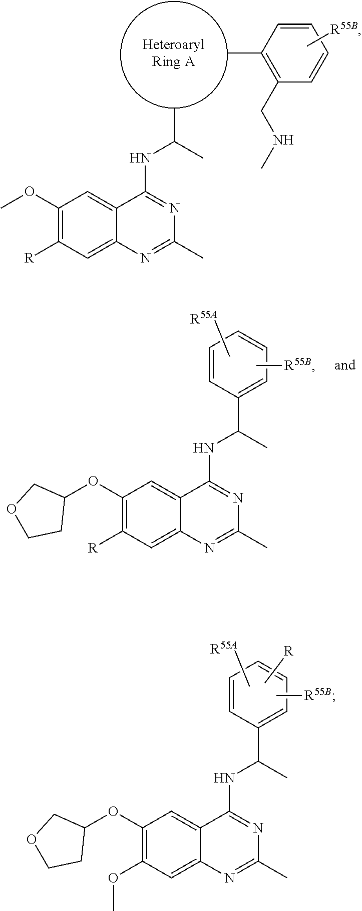

Heterocycle A is a five or six membered heterocycle optionally substituted with an R⁷ 5 group and also optionally substituted with 1 or 2 substituents independently selected from alkyl, haloalkyl, hydroxyl, and halogen.

In certain embodiments, the Targeting Ligand is a Pax2 Targeting Ligand. For example a Targeting Ligand described in E. Grimley, et al. “Inhibition of Pax2 Transcription Activation with a Small Molecule that Targets the DNA Binding Domain.” ACS Chem. Biol. 2017, 12, 10 724-734) or in S. T. J. Bradford, et al. “Identification of Pax protein inhibitors that suppress target gene expression and cancer cell proliferation.” Cell Chem. Biol. 2022, 29, 1-11).

In certain embodiments the Pax2 Targeting Ligand is of Formula:

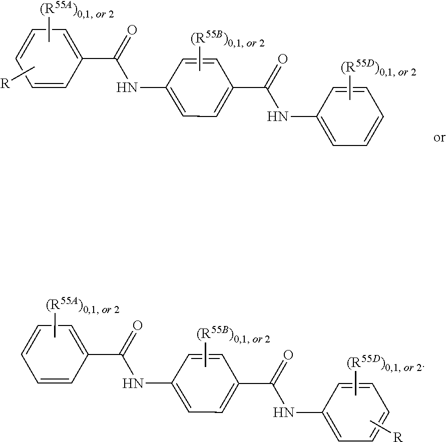

In certain embodiments the Pax2 Targeting Ligand is selected from:

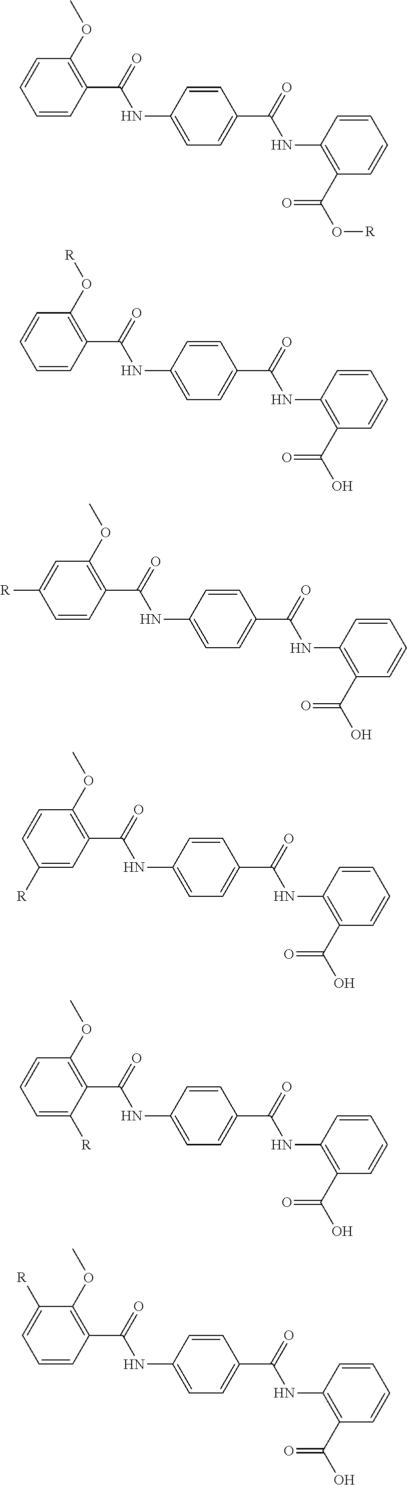

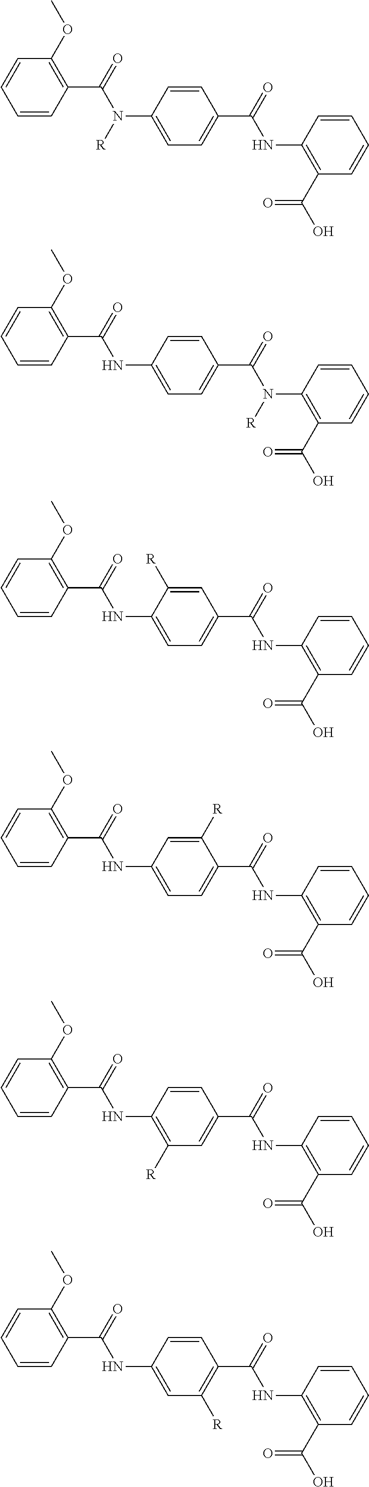

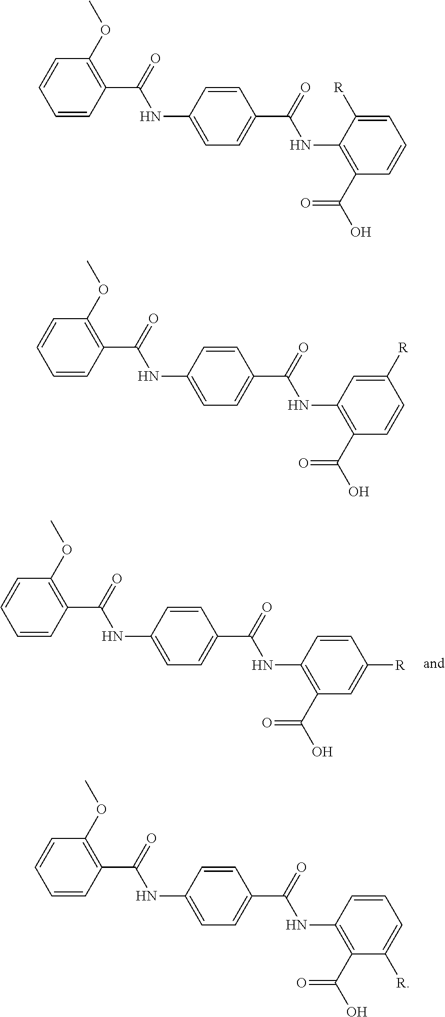

In certain embodiments the Pax2 Targeting Ligand is selected from:

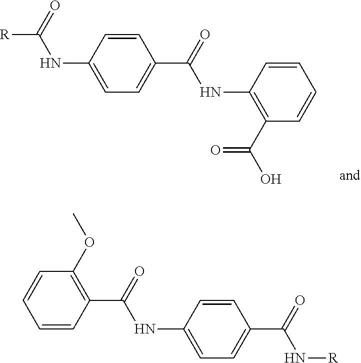

In certain embodiments the Pax2 Targeting Ligand is selected from:

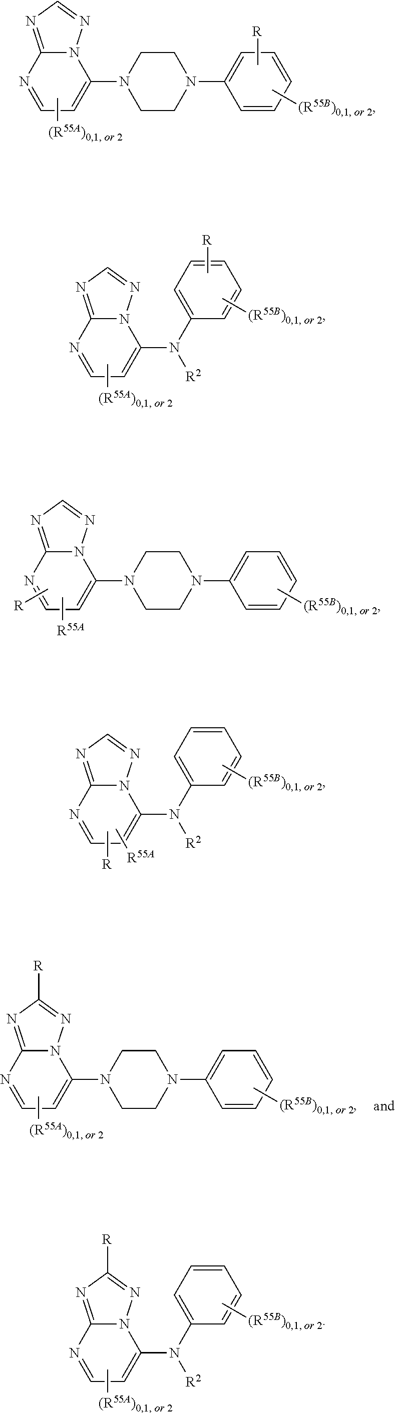

In certain embodiments the Pax2 Targeting Ligand is selected from:

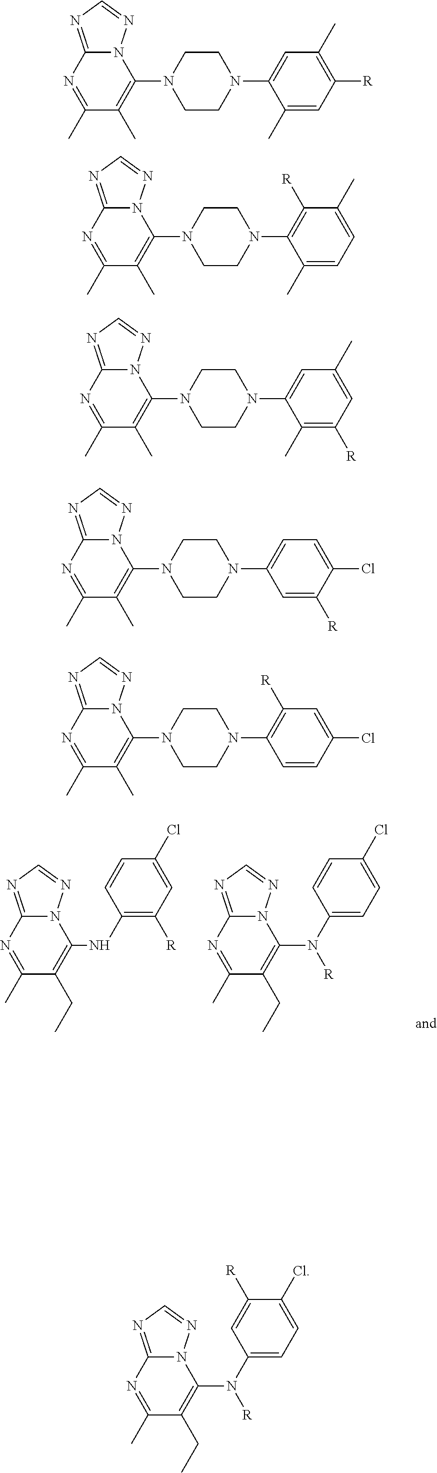

According to the present invention, the Targeting Ligand can be covalently bound to^{P}GP-⁶²¹,C1 the Linker in any manner that achieves the desired results of the Degrader for therapeutic use.

In certain non-limiting embodiments, the Targeting Ligand is bound to the Linker with a functional group that does not adversely affect the binding of the Ligand to the Target Protein.

The attachment points below are exemplary in nature and one of ordinary skill in the art would 10 be able to determine different appropriate attachment points.

The non-limiting compounds described below exemplify some of the members of these types of Targeting Ligands. In the Tables below, R is the point at which the Linker is attached to the Targeting Ligand.

In certain embodiments, the Targeting Ligand binds to ASH1L. For example, the 15 ASH1L small molecule inhibitor may be as described in WO2017/197240, the entirety of which is incorporated herein by reference. In one embodiment, the Targeting Ligand is

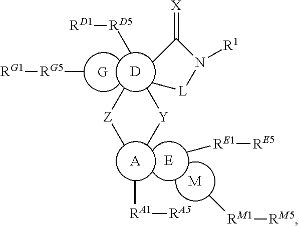

wherein all variables are as defined in WO2017/197240. As described in the 240 application, in some embodiments, any of formulas provided therein may be converted to bifunctional compounds composed of ASH1L inhibitor and an E3 ubiquitin ligase ligand connected with a linker, which function to bind ASH1L and recruit an E ubiquitin ligase (Cereblon, VHL ligase, etc.) complex to ubiquitinate and induce proteasome-mediated degradation of ASH1L. In the present invention, the linker is a Linker as defined herein covalently bound to a Degron as described herein.

In another embodiment, the Targeting Ligand is a deubiquitylating enzyme (DUB) inhibitor as described in WO2018/065768, WO2018/060742, WO2018/060691, WO2018/060689, WO2017/163078, WO2017/158388, WO2017/158381, WO2017/141036, WO2017/103614, WO2017/093718, WO2017/009650, WO2016/156816, or WO2016/046530.

In certain embodiments the Targeting Ligand is a WDR5 Targeting Ligand.

In certain embodiments the Targeting Ligand is a WDR5 Targeting Ligand selected from:

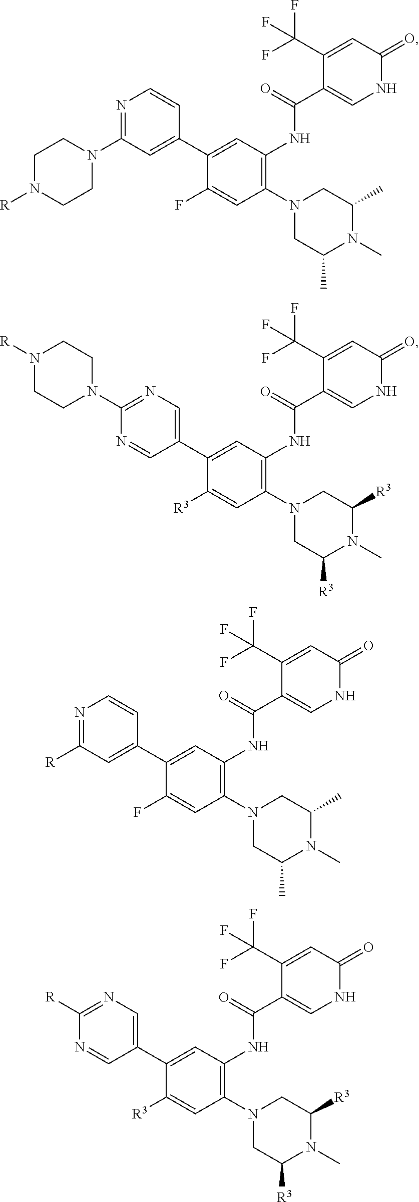

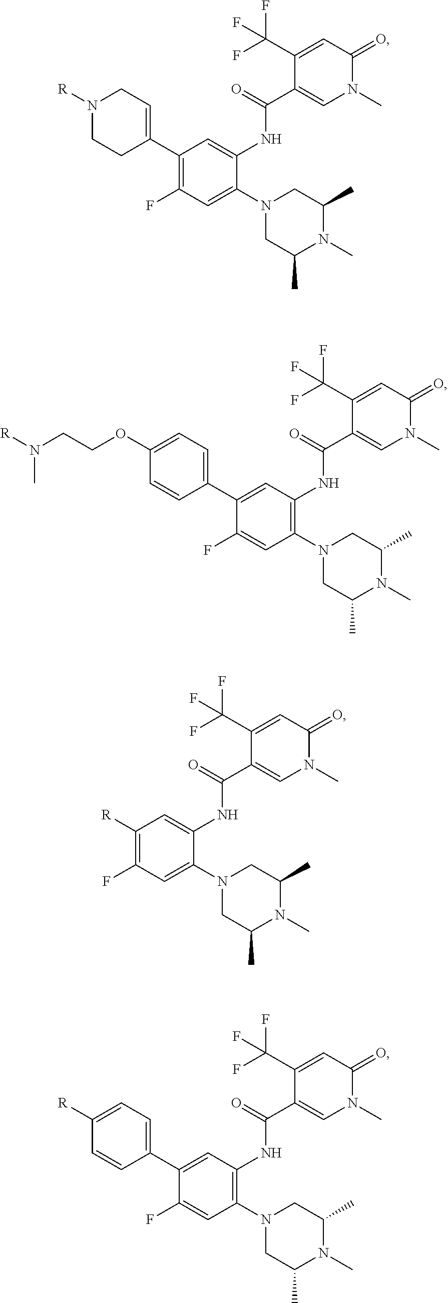

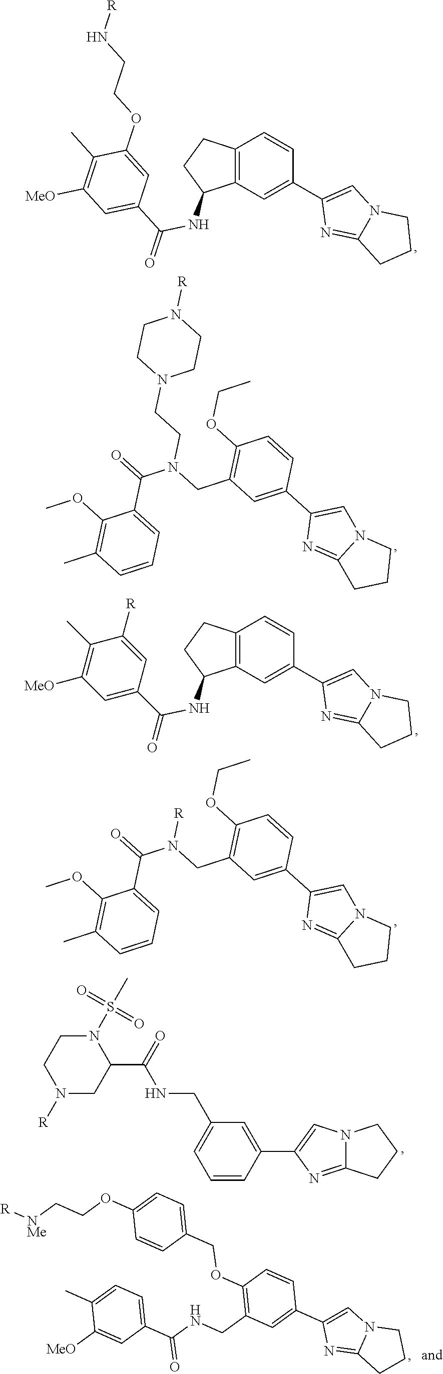

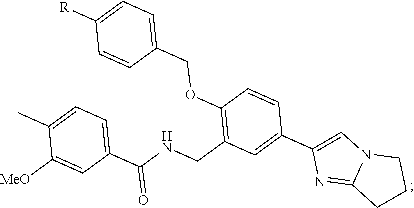

R³ is hydrogen, alkyl, haloalkyl or halogen; and all other variables are defined as above.

In an alternative embodiment, any of the Targeting Ligands as described herein or in the figures may be optionally substituted with one or more, for example 1, 2, 3, 4, or 5, groups selected from R²⁰⁶ In certain embodiments R²⁰⁶ is selected from halogen, C₁-C₄ alkyl, C₁-C₄ haloalkyl, 10 NH₂, NH(CH₃), N(CH₃)2, hydroxyl, or methoxy.

In certain aspects the Targeting Ligand is a BRAF Targeting Ligand. In certain embodiments the BRAF Targeting Ligand is of structure

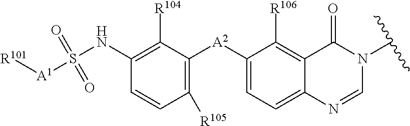

R¹⁰¹ is selected from hydrogen, alkyl and cycloalkyl; R¹⁰² is selected from hydrogen, alkyl, cycloalkyl and haloalkyl;
or R¹⁰¹ and R¹⁰² together with the nitrogen atom to which they are attached form heterocycloalkyl optionally substituted with one or two Rio3; R¹⁰²′ is selected from hydrogen, alkyl, cycloalkyl and haloalkyl;
or R¹⁰¹ and R¹⁰²′ together with the carbon atom to which they are attached form cycloalkyl optionally substituted with one or two R¹⁰³;
each R¹⁰³ is independently selected from hydrogen, halogen (for example F), alkyl, cycloalkyl and alkoxy; R¹⁰⁴ is selected from hydrogen, alkyl, cyano and halogen (for example F); R¹⁰ is selected from hydrogen, alkyl, cyano and halogen (for example F); A² is selected from —O—, —NH— and —(C=O)—; and R¹⁰⁶ is selected from hydrogen, halogen (for example F), hydroxy, amino, 5 dialkylamino, alkoxy, alkyl and alkoxyalkyl.

In certain embodiments A^{l} is NRi⁰² In certain embodiments A^{l} is —CHR¹⁰²′In certain embodiments R¹⁰¹ is hydrogen.

In certain embodiments R¹⁰¹ is alkyl.

In certain embodiments R¹⁰¹ is methyl.

In certain embodiments R¹⁰¹ is cycloalkyl.

In certain embodiments R¹⁰¹ is cyclopropyl.

In certain embodiments R¹⁰² is hydrogen.

In certain embodiments R¹⁰² is alkyl.

In certain embodiments R¹⁰² is methyl.

In certain embodiments R¹⁰² is cycloalkyl.

In certain embodiments R¹⁰² is cyclopropyl.

In certain embodiments R¹⁰² is haloalkyl.

In certain embodiments R¹⁰² is CF₃.

In certain embodiments R¹⁰¹ and R¹⁰² together with the nitrogen atom to which they are attached form a heterocycloalkyl optionally substituted with one or two Rio3 In certain embodiments R¹⁰²′ is hydrogen.

In certain embodiments R¹⁰²′ is alkyl.

In certain embodiments R¹⁰²′ is methyl.

In certain embodiments R¹⁰²′ is cycloalkyl.

In certain embodiments R¹⁰²′ is cyclopropyl.

In certain embodiments R¹⁰²′ is haloalkyl.

In certain embodiments R¹⁰²′ is CF₃.

In certain embodiments each R¹⁰³ is hydrogen.

In certain embodiments each R¹⁰³ is halogen.

In certain embodiments each R¹⁰³ is F.

In certain embodiments each R¹⁰³ is cycloalkyl.

In certain embodiments each R¹⁰³ is alkoxy.

In certain embodiments R¹⁰⁴ is hydrogen.

In certain embodiments R¹⁰⁴ is alkyl.

In certain embodiments R¹⁰⁴ is methyl.

In certain embodiments R¹⁰⁴ is cyano.

In certain embodiments R¹⁰⁴ is halogen.

In certain embodiments R¹⁰⁴ is F.

In certain embodiments R¹⁰⁴ is C1.

In certain embodiments R¹⁰⁵ is hydrogen.

In certain embodiments R¹⁰⁵ is alkyl.

In certain embodiments R¹⁰⁵ is methyl.

In certain embodiments R¹⁰⁵ is cyano.

In certain embodiments R¹⁰⁵ is halogen.

In certain embodiments R¹⁰⁵ is F.

In certain embodiments R¹⁰⁵ is C1.

In certain embodiments A² is -O-.

In certain embodiments A² is —NH-.

In certain embodiments A² is —(C=O)-.

In certain embodiments R¹⁰⁶ is hydrogen.

In certain embodiments R¹⁰⁶ is alkyl.

In certain embodiments R¹⁰⁶ is methyl.

In certain embodiments R¹⁰⁶ is hydroxy.

In certain embodiments R¹⁰⁶ is halogen.

In certain embodiments R¹⁰⁶ is F.

In certain embodiments R¹⁰⁶ is C1.

In certain embodiments R¹⁰⁶ is alkoxy.

In certain embodiments R¹⁰⁶ is methoxy.

VI. METHODS OF TREATMENT The Degrader compounds can be used in an effective amount to treat a host, typically a human, in need thereof, optionally in a pharmaceutically acceptable carrier to treat a disorder 30 described herein.

The terms “treat”, “treating”, and “treatment”, etc., as used herein, refer to any action providing a benefit to a patient for which the present compounds may be administered, including the treatment of any disease state or condition which is modulated through the protein to which the present compounds degrade. Illustrative non-limiting disease states or conditions, including cancer, which may be treated using compounds according to the present invention are set forth hereinabove.

The Degraders and compositions as described herein can be used to degrade a Target Protein which is a mediator of the disorder affecting the patient, such as a human. The control of protein level afforded by the Degraders of the present invention provides treatment of a disease state or condition, which is modulated through the Target Protein by lowering the level of that protein in the cell, e.g., cell of a patient. In certain embodiments, the method comprises administering an effective amount of the compound as described herein, optionally including a pharmaceutically acceptable excipient, carrier, or adjuvant, i.e., a pharmaceutically acceptable composition, optionally in combination with another bioactive agent or combination of agents.

The term “disease state or condition” when used in connection with a Degrader compound is meant to refer to any disease state or condition wherein protein dysregulation (i.e., the amount of protein expressed in a patient is elevated) occurs via a Target Protein and where degradation of such protein in a patient may provide beneficial therapy or relief of symptoms to a patient in need thereof. In certain instances, the disease state or condition may be cured.

The Degrader compounds are for example useful as therapeutic agents when administered in an effective amount to a host, including a human, to treat a myelo- or lymphoproliferative disorder such as B- or T-cell lymphomas, multiple myeloma, Waldenstrom's macroglobulinemia,Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, reactive arthritis, lupus, or type I diabetes; a disease of cardiologic malfunction, including hypercholesterolemia; an infectious disease, including viral and/or bacterial infections; an inflammatory condition, including asthma, chronic peptic ulcers, tuberculosis, rheumatoid arthritis, periodontitis, ulcerative colitis, Crohn's disease, or hepatitis.

The term “disease state or condition” when used in connection with a Degron compound for example, refers to any therapeutic indication which can be treated by decreasing the activity of cereblon or a cereblon-containing E3 Ligase, including but not limited to uses known for the cereblon binders thalidomide, pomalidomide or lenalidomide.

Nonlimiting examples of uses for cereblon binders are multiple myeloma, a hematological disorder such as myelodysplastic syndrome, cancer, tumor, abnormal cellular proliferation, HIV/AIDS, HBV, HCV, SARS-CoV2, hepatitis, Crohn's disease, sarcoidosis, graft-versus-host disease, rheumatoid arthritis, Behcet's disease, tuberculosis, and myelofibrosis. Other indications include a myelo- or lymphoproliferative disorder such as B-or T-cell lymphomas, Waldenstrom's macroglobulinemia, Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, arthritis, and in particular rheumatoid arthritis, lupus, or type I diabetes; a disease of cardiologic malfunction, including hypercholesterolemia; an infectious disease, including viral and/or bacterial infection, as described generally herein; an inflammatory condition, including asthma, chronic peptic ulcers, tuberculosis, rheumatoid arthritis, periodontitis and ulcerative colitis.

In certain embodiments, the present invention provides for administering a Degrader compound to a patient, for example, a human, having an infectious disease, wherein the therapy targets a protein of the infectious agent, optionally in combination with another bioactive agent.

The disease state or condition may be a disease caused by a microbial agent or other exogenous agent such as a virus (as non-limiting examples, HIV, HBV, HCV, HSV, HPV, RSV, CMV, Ebola, SARS-CoV2, Flavivirus, Pestivirus, Rotavirus, Influenza, Coronavirus, EBV, viral pneumonia, drug-resistant viruses, Bird flu, RNA virus, DNA virus, adenovirus, poxvirus, Picornavirus, Togavirus, Orthomyxovirus, Retrovirus or Hepadnovirus), bacteria (Gram-negative, Gram-positive, fungus, protozoa, helminth, worms, prion, parasite, or other microbe or may be a disease state, which is caused by overexpression of a protein, which leads to a disease state and/or condition.

In certain embodiments, the condition treated with a compound of the present invention is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation.

Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, melanoma, basal cell carcinoma and squamous cell carcinoma.

Other hyperproliferative cell disorders include blood vessel proliferation disorders, fibrotic disorders, autoimmune disorders, graft-versus-host rejection, tumors and cancers. Blood vessel proliferative disorders include angiogenic and vasculogenic disorders.

Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix.

Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be 10 caused by viral infection such as hepatitis. Lipocytes appear to play a major role in hepatic cirrhosis.

Mesangial disorders are brought about by abnormal proliferation of mesangial cells.

Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis.

Immunologically mediated leukocyte infiltration also occurs at sites other than the skin, such as in the airways in asthma and in the tear producing gland of the eye in keratoconjunctivitis sicca.

In one non-limiting embodiment compounds of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Disease states of conditions which may be treated using compounds according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease 1 (PKD1) or 2 (PKD2) Prader-Willi syndrome, Sickle-cell disease, Tay-Sachs disease, Turner syndrome.

Further disease states or conditions which may be treated by compounds according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by compounds according to the present invention include aceruloplasminemia, Achondrogenesis type II, achondroplasia, Acrocephaly, Gaucher disease type 2, acute intermittent porphyria, Canavan disease, Adenomatous Polyposis Co/i, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstrom syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia-familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Weissenbacher-Zweymuller syndrome, Wolf-Hirschhorn syndrome, Wolff Periodic disease, Weissenbacher-Zweymuller syndrome and Xeroderma pigmentosum, among others.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors.

Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, colorectal cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis;; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, 5 Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In certain embodiments the cancer is an unresectable cancer.

Unresectable cancers are cancers that cannot be removed (resected) by surgery. Many cancers can be either resectable or unresectable depending on the site of the tumor and the size of the tumor.

In certain embodiments an unresectable cancer is treated with an effective amount of a compound described herein or a pharmaceutically acceptable salt thereof.

In other embodiments resectable cancer is treated with a compound described herein or a pharmaceutically acceptable salt thereof wherein the treatment additionally optionally includes surgically removing the tumor.

Locally advanced cancers are cancers that have grown outside of the body part where the tumor started but have not yet spread (metastasized) to other parts of the body.

In certain embodiments a locally advanced cancer is treated with an effective amount of a compound described herein or a pharmaceutically acceptable salt thereof.

In one embodiment the cancer is NUT midline cardinioma.

In one embodiment the cancer is adenoid cystic carcinoma.

The term “bioactive agent” is used to describe an agent, other than a compound 30 according to the present invention, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

VII. COMBINATION THERAPY The selected Degron or Degrader compound of the present invention can be used in an effective amount alone or in combination with another therapeutic or bioactive agent to treat a host such as a human with a disorder as described herein.

The term “bioactive agent” is used to describe an agent, other than the selected compound according to the present invention, which can be used in combination or alternation with a compound of the present invention to achieve a desired result of therapy. In one embodiment, the compound of the present invention and the bioactive agent are administered in a manner that they are active in vivo during overlapping time periods, for example, have time-period overlapping Cmax, Tmax, AUC or other pharmacokinetic parameter. In another embodiment, the compound of the present invention and the bioactive agent are administered to a host in need thereof that do not have overlapping pharmacokinetic parameter, however, one has a therapeutic impact on the therapeutic efficacy of the other.

In some aspects of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro), and the PD-Li/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and MedImmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In other embodiments, an active compounds described herein can be administered in an effective amount for the treatment of abnormal tissue of the female reproductive system such as breast, ovarian, endometrial, or uterine cancer, in combination or alternation with an effective amount of an estrogen inhibitor including but not limited to a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors.

Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703, 810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestratnt; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In another embodiment, an active compounds described herein can be administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor degrader, a complete androgen receptor degrader, or another form of partial or complete androgen antagonist. In one embodiment, the prostate or testicular cancer is androgen-resistant.

Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In one embodiment, the bioactive agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113,.

In one embodiment, the bioactive agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In one embodiment, the bioactive agent is an HER-2 inhibitor. Examples of HER-2 inhibitors include trastuzumab, lapatinib, ado-trastuzumab emtansine, and pertuzumab.

In one embodiment, the bioactive agent is a CD20 inhibitor. Examples of CD20 inhibitors include obinutuzumab, rituximab, fatumumab, ibritumomab, tositumomab, and ocrelizumab.

In one embodiment, the bioactive agent is a JAK3 inhibitor. Examples of JAK3 inhibitors include tasocitinib.

In one embodiment, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]—N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]—N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[l,l-biphenyl]-2-yl)methyl)piperazin-1-yl)—N-((4-((4-morpholino-1-(phenylthio)butan-2-yl)amino)-3-((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (Oblimersen).

In one embodiment, the bioactive agent is a kinase inhibitor. In one embodiment, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 30 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4-methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)—N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]—N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,1OR,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4.5 h]isochromen-l0-yl]acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422), and the structure described in WO2014/071109.

Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R-N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)—N-(3-(8-(phenylamino)imidazo[1,2-a]pyrazin-6-yl)phenyl)benzamide, CGI-1746 (4-(tert-butyl)—N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)—N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-iH-pyrazol-4-yl)benzo[h][1,6]naphthyridin-2(1H)-one), and RN486 (6-cyclopropyl-8-fluoro-2-(2-hydroxymethyl-3-f{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)—N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]—N-(4-methyl-3-f{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306(see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J.

Med. Chem. 2012, 55, 3614-3643 incorporated in its entirety herein), fisetin (see Singh et al.

Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In one embodiment, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAI 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]- benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-iH-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)—N-(2-hydroxyethoxy)-5-((3-oxo-1,2-oxazinan-2y1)methyl)benzamide), or AZD8330 (2-((2-fluoro-4-iodophenyl)amino)—N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, 15 PD98059, BIX 02189, BIX 02188, binimetinib, SL-327, TAK-733, PD318088.

In one embodiment, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]—N-methylpyridine-2-carboxamide;4-methylbenzenesulfonate), AZ628 (3-(2-cyanopropan-2-yl)—N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)—N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]—N-Methyl-2pyridinecarboxaMide 1-Oxide), PLX-4720, dabrafenib (GSK2118436), GDC-0879, RAF265, AZ 628, Sf590885, ZM336372, GW5074, TAK-632, CEP-32496, LY3009120, and GX818 (Encorafenib).

In one embodiment, the bioactive agent is an AKT inhibitor, including but not limited to, MK-2206, GSK690693, Perifosine, (KRX-0401), GDC-0068, Triciribine, AZD5363, Honokiol, PF-04691502, and Miltefosine, a FLT-3 inhibitor, including but not limited to, P406, Dovitinib, Quizartinib (AC220), Amuvatinib (MP-470), Tandutinib (MLN518), ENMD-2076, and KW-2449, or a combination thereof.

In one embodiment, the bioactive agent is an mTOR inhibitor. Examples of mTOR inhibitors include but are not limited to rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib), refametinib/BAY869766/RDEAl19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-iH-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)—N-(2-hydroxyethoxy)-5-((3-oxo-1,2-oxazinan-2-yl)methyl)benzamide), or AZD8330 (2-((2-fluoro-4-iodophenyl)amino)—N-(2-hydroxyethoxy)-1,5-dimethyl-6-oxo-1,6-dihydropyridine-3-carboxamide).

In one embodiment, the bioactive agent is a RAS inhibitor. Examples of RAS inhibitors 25 include but are not limited to Reolysin and siGl2D LODER.

In one embodiment, the bioactive agent is a HSP inhibitor. HSP inhibitors include but are not limited to Geldanamycin or 17—N-Allylamino-17-demethoxygeldanamycin (17AAG), and Radicicol.

Additional bioactive compounds include, for example, everolimus, trabectedin, 30 abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhbitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, 5 CC 8490, cilengitide, gimatecan, IL13-PE38QQR, INO 1001, IPdRi KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In one embodiment, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™) Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-a, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon a-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with a Degrader disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In one aspect of the invention, the disclosed compound is administered in combination with an anti-infective agent, for example but not limited to an anti-HIV agent, anti-HCV agent, anti-HBV agent, or other anti-viral or anti-bacterial agent. In one embodiment, the anti-HIV agent can be, but is not limited to, for example, a nucleoside reverse transcriptase inhibitor (NRTI), other non-nucloeoside reverse transcriptase inhibitor, protease inhibitor, fusion inhibitor, among others.

Nucleoside/Nucleotide Reverse Transcriptase Inhibitors (NRTIs) include, but are not limited to, Abacavir or ABC (Ziagen), Didanosine or ddl (Videx), Emtricitabine or FTC (Emtriva), Lamivudine or 3TC (Epivir), ddC (zalcitabine), Stavudine or d4T (Zerit), Tenofovircor TDF (Viread), D-D4FC (Reverset), and Zidovudine or AZT or ZDV (Retrovir).

Non-nucleoside Reverse Transcriptase Inhibitors (NNRTIs) include, but are not limited to, Delavirdine (Rescriptor), Efavirenz (Sustiva), Etravirine (Intelence), Nevirapine (Viramune), and Rilpivirine (Edurant). Anti-HIV Protease Inhibitors (PIs) include, but are not limited to, Atazanavir or ATV (Reyataz), Darunavir or DRV (Prezista), Fosamprenavir or FPV (Lexiva), Indinavir or IDV (Crixivan), Lopinavir+ritonavir, or LPV/r (Kaletra), Nelfinavir or NFV (Viracept), Ritonavir or RTV (Norvir), Saquinavir or SQV (Invirase), Tipranavir, or TPV (Aptivus), Cobicistat (Tybost), Atazanavir+cobicistat, or ATV/COBI (Evotaz), Darunavir+cobicistat, or DRV/COBI (Prezcobix).

Anti-HIV Fusion Inhibitors include, but are not limited to, Enfuvirtide or ENF or T-20 (Fuzeon). Anti-HIV also include, but are not limited to, Maraviroc or MVC (Selzentry).

Anti-HIV Integrase Inhibitors include, but are not limited to Dolutegravir (Tivicay), Elvitegravir (Vitekta), Raltegravir (Isentress).

Anti-HIV combinations agents include Abacavir+Dolutegravir+lamivudine,or ABC/DTG/3TC (Triumeq), Abacavir+lamivudine or ABC/3TC (Epzicom), Abacavir+lamivudine+zidovudine, or ABC/3TC/ZDV (Trizivir), Efavirenz+emtricitabine+tenofovir or EFV/FTC/TDF (Atripla, Tribuss), elvitegravir, cobicistat, emtricitabine, tenofovir alafenamide or EVG/COBI/FTC/TAF or ECF/TAF (Genvoya; (Stribild), emtricitabine+rilpivirine+tenofovir or FTC/RPV/TAF (Odefsey); Emtricitabine+rilpivirine+tenofovir or FTC/RPV/TDF (Complera), Emtricitabine+tenofovir or TAF/FTC (Descovy), emtricitabine and tenofovir disoproxil fumarate (Truvada), and Lamivudine+zidovudine or 3TC/ZDV (Combivir).

Other anti-HIV compounds include, but are not limited to Racivir, L-FddC, L-FD4C, 15 SQVM (Saquinavir mesylate), IDV (Indinavir), SQV (Saquinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used in co-administration with the disclosed compounds according to the present invention. NNRTIs may be selected from the group consisting of nevirapine (BI-R6-587), delavirdine (U-90152S/T), efavirenz (DMP-266), UC-781 (N-[4-chloro-3-(3-methyl-2-butenyloxy)phenyl]-2methyl3-furancarbothiamide), etravirine (TMC125), Trovirdine (Ly300046.HCl), HI-236, HI-240, HI-280, HI-281, rilpivirine (TMC-278), MSC-127, HBY 097, DMP266, Baicalin (TJN-151) ADAM-II (Methyl 3′,3′-dichloro-4′,4″-dimethoxy-5′,5″-bis(methoxycarbonyl)-6,6-diphenylhexenoate), Methyl 25 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, S-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S(1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]—N′-(2-thiazolyl)thiourea (PETT C1, F derivative), N-{2-(2,6-Difluorophenethyl]—N′-[2-(5-bromopyridyl)]thiourea {PETT derivative), N-{2-(2,6-Difluorophenethyl]—N′-[2-(5-methylpyridyl]thiourea {PETT Pyridyl derivative), N-[2-(3-Fluorofuranyl)ethyl]—N′-[2-(5-chloropyridyl)]thiourea, N-[2-(2-Fluoro-6-ethoxyphenethyl)]—N′-[2-(5-bromopyridyl)]thiourea, N-(2-Phenethyl)—N′-(2-thiazolyl)thiourea (LY-73497), L-697,639, L-697,593, L-697,661, 342-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, 5-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5 (9bH)-one, Tivirapine (R86183), UC-38 and UC-84, among others.

In one aspect of the invention, the disclosed compound when used to treat an HCV infection can be administered in combination with another anti-HCV agent. Anti-HCV agents are known in the art. To date, a number of fixed dose drug combinations have been approved for the treatment of HCV. Harvoni® (Gilead Sciences, Inc.) contains the NS5A inhibitor ledipasvir and the NS5B inhibitor sofosbuvir. Technivie™ (AbbVie, Inc.) is a fixed-dose combination containing ombitasvir, an NS5A inhibitor; paritaprevir, an NS3/4A protease inhibitor; and ritonavir, a CYP3A inhibitor. Daklinza™ (daclatasvir, Bristol-Myers Squibb) is an HCV NS5A inhibitor indicated for use with sofosbuvir for the treatment of chronic genotype 3 infection. Zepatier™ (Merck & Co.) has recently been approved for the treatment of chronic HCV genotypes 1 and 4. Zepatier™ is a fixed-dose combination product containing elbasvir, an HCV NS5A inhibitor, and grazoprevir, an HCV NS3/4A protease inhibitor. Zepatier™ is indicated with or without ribavirin. Epclusa® (Gilead Sciences, Inc.) is a fixed-dose combination tablet containing sofosbuvir and velpatasvir. Additional anti-HCV agents and combinations thereof include those described in U.S. Pat. Nos. 9,382,218; 9,321,753; 9,249,176; 9,233,974; 9,221,833; 9,211,315; 9,194,873; 9,186,369; 9,180,193; 9,156,823; 9,138,442; 9,133,170; 9,108,999; 9,090,559; 9,079,887; 9,073,943; 9,073,942; 9,056,090; 9,051,340; 9,034,863; 9,029,413; 9,011,938; 8,987,302; 8,945,584; 8,940,718; 8,927,484; 8,921,341; 8,884,030; 8,841,278; 8,822,430; 8,772,022; 8,765,722; 8,742,101; 8,741,946; 8,674,085; 8,673,288; 8,669,234; 8,663,648; 8,618,275; 8,580,252; 8,575,195; 8,575,135; 8,575,118; 8,569,302; 8,524,764; 8,513,298; 8,501,714; 8,404,651; 8,273,341; 8,257,699; 8,197,861; 8,158,677; 8,105,586; 8,093,353; 8,088,368; 7,897,565; 7,871,607; 7,846,431; 7,829,081; 7,829,077; 7,824,851; 7,572,621; and 7,326,536; Patents assigned to Alios: U.S. Pat. Nos. 9,365,605; 9,346,848; 9,328,119; 9,278,990; 9,249,174; 9,243,022; 9,073,960; 9,012,427; 8,980,865; 8,895,723; 8,877,731; 8,871,737; 8,846,896 and 8,772,474; Achillion 9,273,082; 9,233,136; 9,227,952; 9,133,115; 9,125,904; 9,115,175; 9,085,607; 9,006,423; 8,946,422; 8,835,456; 8,809,313; 8,785,378; 8,614,180; 8,445,430; 8,435,984; 8,183,263; 8,173,636; 8,163,693; 8,138,346; 8,114,888; 8,106,209; 8,088,806; 8,044,204; 7,985,541; 7,906,619; 7,902,365; 7,767,706; 7,741,334; 7,718,671; 7,659,399; 7,476,686; 7,439,374; 7,365,068; 7,199,128; and 7,094,807; Cocrystal Pharma Inc. 9,181,227; 9,173,893; 9,040,479 and 8,771,665; Gilead Sciences 9,353,423; 9,346,841; 9,321,800; 9,296,782; 9,296,777; 9,284,342; 9,238,039; 9,216,996; 9,206,217; 9,161,934; 9,145,441; 9,139,604; 9,090,653; 9,090,642; 9,085,573; 9,062,092; 9,056,860; 9,045,520; 9,045,462; 9,029,534; 8,980,878; 8,969,588; 8,962,652; 8,957,046; 8,957,045; 8,946,238; 8,933,015; 8,927,741; 8,906,880; 8,889,159; 8,871,785; 8,841,275; 8,815,858; 8,809,330; 8,809,267; 8,809,266; 8,779,141; 8,765,710; 8,759,544; 8,759,510; 8,735,569; 8,735,372; 8,729,089; 8,722,677; 8,716,264; 8,716,263; 8,716,262; 8,697,861; 8,664,386; 8,642,756; 8,637,531; 8,633,309; 8,629,263; 8,618,076; 8,592,397; 8,580,765; 8,569,478; 8,563,530; 8,551,973; 8,536,187; 8,513,186; 8,513,184; 8,492,539; 8,486,938; 8,481,713; 8,476,225; 8,420,597; 8,415,322; 8,338,435; 8,334,270; 8,329,926; 8,329,727; 8,324,179; 8,283,442; 8,263,612; 8,232,278; 8,178,491; 8,173,621; 8,163,718; 8,143,394; patents assigned to Idenix, acquired by Merck, include U.S. Pat. Nos. 9,353,100; 9,309,275; 9,296,778; 9,284,307; 9,249,173; 9,243,025; 9,211,300; 9,187,515; 9,187,496, 9,109,001; 8,993,595; 8,951,985; 8,691,788; 8,680,071; 8,637,475; 8,507,460; 8,377,962; 8,362,068; 8,343,937; 8,299,038; 8,193, 372; 8,093,379; 7,951,789; 7,932,240; 7,902,202; 7,662,798; 7,635,689; 7,625,875; 7,608,600; 7,608,597; 7,582,618; 7,547,704; 7,456,155; 7,384,924; 7,365,057; 7,192,936; 7,169,766; 7,163,929; 7,157,441; 7,148,206; 7,138,376; 7,105,493; 6,914,054 and 6,812,219; patents assigned to Merck include U.S. Pat. Nos. 9,364,482; 9,339,541; 9,328,138; 9,265,773; 9,254,292; 9,243,002; 9,242,998; 9,242,988; 9,242,917; 9,238,604; 9,156,872; 9,150,603; 9,139,569; 9,120,818; 9,090,661; 9,073,825; 9,061,041; 8,987,195; 8,980,920; 8,927,569; 8,871,759; 8,828,930; 8,772,505; 8,715,638; 8,697,694; 8,637,449; 8,609,635; 8,557,848; 8,546,420; 8,541,434; 8,481,712; 8,470,834; 8,461,107; 8,404,845; 8,377,874; 8,377,873; 8,354,518; 8,309,540; 8,278,322; 8,216,999; 8,148,349; 8,138,164; 8,080,654; 8,071,568; 7,973,040; 7,935,812; 7,915,400; 7,879,815; 7,879,797; 7,632,821; 7,569,374; 7,534,767; 7,470,664 and 7,329,732; patent application publication US 2013/0029904 to Boehringer Ingelheim GMBH and US 2014/0113958 to Stella Aps.

In one embodiment, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g.ridaforolimus, azathioprine, campath 1H, a SiP receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3@), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA4lg (Abatacept), belatacept, LFA3lg,, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments a monotherapy or combination described herein additionally comprises administering one or more additional therapeutic agents to decrease side effects of the therapy. For example, in certain embodiments a compound described herein or a pharmaceutically acceptable salt thereof is administered concurrently, before, or after administration of an antineutropenia medication, antinausea medication, an antihistamine, and/or an antipain medication. Non-limiting examples of antineutropenia medications include growth factors for example a granulocyce colony stimulating factor (G-CSF). In certain embodiments a therapy in a table above is administered in combination with a G-CSF. G-CSF (or another active agent) can be given before, with after, or on different days than the compound of the present invention.

Non-limiting examples of granulocyte colony stimulating factors include filgrastim (in the form of neupogen, zarxio, nivestym, or another form), CG-10639, and PEGF.

In certain embodiments the granulocyte colony stimulating factor is pegfilgrastim. In certain embodiments the granulocyte colony stimulating factor is Neulasta. In certain embodiments the granulocyte colony stimulating factor is selected from Ristempa, Tezmota, Fulphila, Pelgraz, Udenyca, Udenyca, Pelmeg, Ziextenzo, Grasustek, Ziextenzo, Lapelga, Neutropeg, Cegfila, Nyvepria, and Stimufend.

In certain embodiments the therapy described herein further comprises an antinausea medication. Non-limiting examples of antinauasea medications include aprepitant, dolasetron, granisetron, ondansetron, palonosetron, proclorperazine, promethazine, netupitant-palonosetron, rolapitant, lorazepam, metoclopramide, famotidine, dexamethasone, and ranitidine.

In certain embodiments the therapy described herein further comprises an antihistamine medication. Non-limiting examples of antihistamine medications include benadryl, cetirizine, loratadine, and fexofenadine.

In certain embodiments the therapy described herein further comprises an antipain medication. Non-limiting examples of antipain medications include tramadol, hydromorphone, methadone, morphine, oxycodone, hydrocodone, oxymorphone, fentanyl, and tapentadol. VIII. PHARMACEUTICAL COMPOSITIONS The Degron compound or Degrader as disclosed herein can be administered as the neat chemical, but are more typically administered as a pharmaceutical composition, that includes an effective amount for a host, typically a human, in need of such treatment for any of the disorders described herein. Accordingly, the disclosure provides pharmaceutical compositions comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

As used herein, “pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 75, 100, 200, 250, 300, 400, 500, 600, 700, or 750 mg of active compound, or its salt. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

In certain aspects the compound or a pharmaceutical composition is administered parenterally. Parenteral administration includes, e.g., subcutaneous (s.c.), intravenous (i.v.), intramuscular (i.m.), intrasternal injection, or infusion techniques.

The pharmaceutical composition may also include a molar ratio of the active compound and an additional active agent. For example, the pharmaceutical composition may contain a molar ratio of about 0.5:1, about 1:1, about 2:1, about 3:1 or from about 1.5:1 to about 4:1 of an anti-inflammatory or immunosuppressing agent.

Compounds disclosed herein may be administered orally, topically, parenterally, by inhalation or spray, sublingually, via implant, including ocular implant, transdermally, via buccal administration, rectally, as an ophthalmic solution, injection, including ocular injection, intraveneous, intra-aortal, intracranial, subdermal, intraperitioneal, subcutaneous, transnasal, sublingual, or rectal or by other means, in dosage unit formulations containing conventional pharmaceutically acceptable carriers.

For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated.

The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound. Formulations suitable for rectal administration are typically presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

In certain aspects the pharmaceutical composition comprises a pharmaceutically acceptable salt of a compound of the present invention. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound 5 formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, formic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, trifluoroacetic (TFA), HOOC-(CH₂)n-COOH where n is 0-4, and the like, or using a different acid that produces the same counterion.

Lists of additional suitable salts may be found, e.g., in Remington 's Pharmaceutical Sciences, 17th ed., Mack Publishing Company, Easton, Pa., p. 1418 (1985).

IX. SYNTHETIC METHODS General Synthesis The compounds described herein can be prepared by methods known to those skilled in the art. In some non-limiting examples, the disclosed compounds can be made by the 20 schemes provided below.

Compounds of the present invention with stereocenters may be drawn without stereochemistry for convenience. One skilled in the art will recognize that pure enantiomers and diastereomers can be prepared by methods known in the art. Examples of methods to obtain optically active materials include at least the following.
i) physical separation of crystals-a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization-a technique whereby the individual enantiomers are 30 separately crystallized from a solution of the racemate, possible only if the latter is a conglomerate in the solid state;
iii) enzymatic resolutions-a technique whereby partial or complete separation of a racemate by virtue of differing rates of reaction for the enantiomers with an enzyme; iv) enzymatic asymmetric synthesis-a synthetic technique whereby at least one step of the synthesis uses an enzymatic reaction to obtain an enantiomerically pure or enriched synthetic precursor of the desired enantiomer;
v) chemical asymmetric synthesis-a synthetic technique whereby the desired enantiomer is synthesized from an achiral precursor under conditions that produce asymmetry (i.e., chirality) in the product, which may be achieved using chiral catalysts or chiral auxiliaries; vi) diastereomer separationsa technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations-a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer; viii) kinetic resolutions-this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors-a synthetic technique whereby the desired enantiomer is obtained from non-chiral starting materials and where the stereochemical integrity is not or is only minimally compromised over the course of the synthesis;
x) chiral liquid chromatography-a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions; xi) chiral gas chromatography-a technique whereby the racemate is volatilized and enantiomers are separated by virtue of their differing interactions in the gaseous mobile phase with a column containing a fixed non-racemic chiral adsorbent phase;
xii) extraction with chiral solventsa technique whereby the enantiomers are separated by virtue of preferential dissolution of one enantiomer into a particular chiral solvent; xiii) transport across chiral membranes-a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the 5 non-racemic chiral nature of the membrane that allows only one enantiomer of the racemate to pass through.
xiv) simulated moving bed chromatography, is used in one embodiment. A wide variety of chiral stationary phases are commercially available.

The chemical synthesis of Degraders of the present invention may be carried out 10 according to the following general Schemes 1-4 which differ in sequence of steps used for binding a degron, linker, and targeting ligand. As illustrated in these Schemes and Examples below, compounds for use in the present invention can readily be synthesized by one skilled in the art in a variety of methods and chemical reactions.

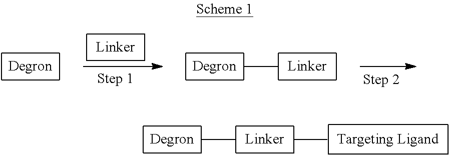

As shown in Scheme 1, compounds for use in the present invention can be prepared by chemically combining a Degron and a Linker followed by subsequent addition of a Targeting Ligand.

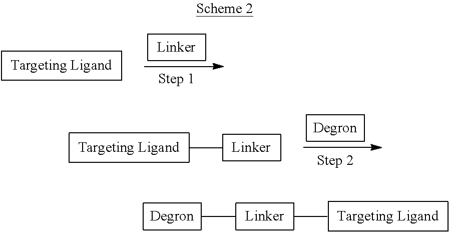

Similarly, in Scheme 2 compounds for use in the present invention are prepared by chemically combing a Targeting Ligand and Linker first, followed by subsequent addition of a Degron.

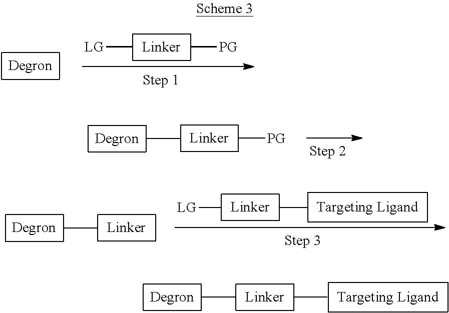

In Scheme 3, in Step 1, a nucleophilic Degron displaces a leaving group (LG) on the^{PG}62⁷,0 Linker to make a Degron Linker fragment. In Step 2, the protecting group (PG) is removed by 5 methods known in the art to free a nucleophilic site on the linker. In Step 3, the nucleophilic Degron Linker fragment displaces a leaving group on the Targeting Ligand to form a compound for use in the present invention. In an alternative embodiment Step 1 and/or Step 2 is accomplished by a coupling reaction instead of a nucleophilic attack.

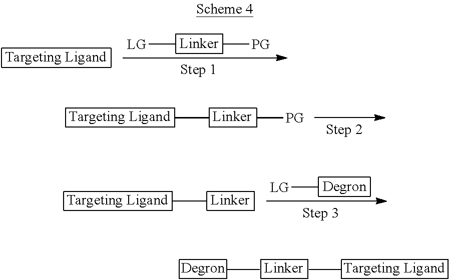

In Scheme 4, in Step 1, a nucleophilic Targeting Ligand displaces a leaving group on the Linker to make a Targeting Ligand Linker fragment. In Step 2, the protecting group is removed by methods known in the art to free a nucleophilic site on the linker. In Step 3, the nucleophilic Targeting Ligand Linker fragment displaces a leaving group on the Degron to 15 form a compound for use in the present invention. In an alternative embodiment Step 1 and/or Step 2 is accomplished by a coupling reaction instead of a nucleophilic attack.

### EXAMPLE 1: SYNTHESIS OF CRBN LIGANDS

Synthesis A1: Synthesis of 3-((2,6-dioxopiperidin-3-yl)oxy)benzenesulfonyl fluoride (A-1)

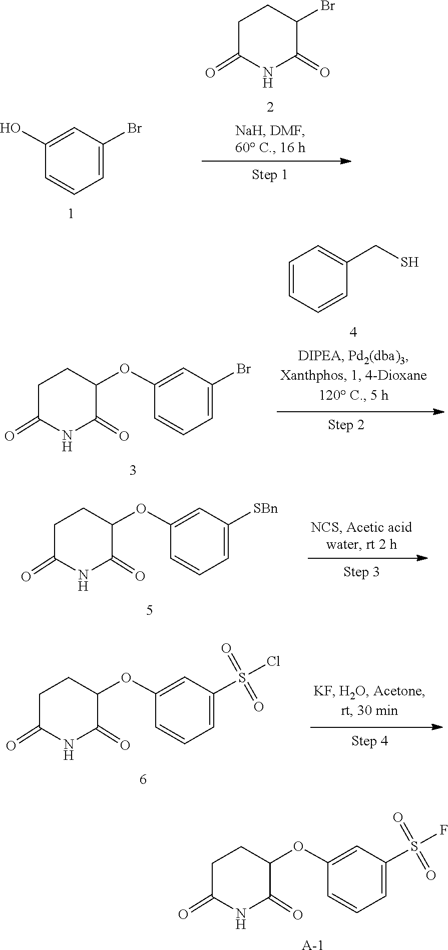

Step-1: To a stirred solution of 3-bromophenol 1 (2.0 g, 11.6 mmol) in dimethylformamide (20 mL) was added NaH (832 mg, 34.7 mmol) at 0° C. and stirred for 30 minutes. To the reaction mixture, 3-bromopiperidine-2,6-dione 2 (4.44 g, 23.12 mmol) was added and the solution was 10 stirred for 16 hours at 60° C. Upon completion of the reaction, the reaction mixture was quenched with saturated ammonium chloride (10 mL) and extracted with ethyl acetate (2×25 mL). The combined organic layers were dried over anhydrous sodium sulfate, filtered, and concentrated under reduced pressure to afford the crude product. The crude product was purified by column chromatography (Davisil silica, 50% ethyl acetate in petroleum ether as an eluent) to afford 3-(3-bromophenoxy) piperidine-2,6-dione 3 (1 g, 24% yield) colorless oil.

LCMS (ES*): m z 284.05 [M+H]+.

Step-2: To a stirred solution of 3-(3-bromophenoxy) piperidine-2,6-dione 3 (1 g, 3.5 mmol) and phenylmethanethiol 4 (656 mg, 5.3 mmol) in dioxane (3.2 mL) were added N,N-diisopropylethylamine (1.4 g, 10.6 mmol, 1.8 mL), xantphos (407 mg, 704 mol) and purged with argon gas for 5 minutes. After purging, Tris(dibenzylideneacetone)dipalladium (3.22 g, 3.52 mmol) was added and the reaction mixture was stirred at 120° C. for 5 hours. After completion of reaction, the reaction mixture was filtered through a celite bed and washed with ethyl acetate (20 mL). The filtrate was concentrated under reduced pressure to afford the crude product. The crude product was purified by column chromatography (Davisil silica, 100% ethyl acetate as an eluent) to afford 3-(3-benzylsulfanylphenoxy) piperidine-2,6-dione 5 (0.4 g, 22% yield) as pale-yellow oil. LCMS (ES*): m z 328.23 [M+H]+.

Step-3: To a stirred solution of 3-(3-benzylsulfanylphenoxy)piperidine-2,6-dione 5 (0.2 g, 611 mol) in acetic acid (37 mg, 611 mol, 35 L) and water (469 L) was added N-chlorosuccinimide (245 mg, 1.8 mmol) at 0° C. The reaction mixture was warmed to room temperature and stirred for 2 hours. After completion, the reaction was quenched with saturated sodium bicarbonate solution (10 mL) and extracted with ethyl acetate (2×10 mL). The combined organic layer was dried over sodium sulfate and concentrated under reduced pressure to afford crude 3-[(2,6-dioxo-3-piperidyl) oxy]benzenesulfonyl chloride 6 (0.2 g, 59% yield) as a colorless clear oil. LCMS (ES-): m z 301.97 [M-H]-.

Step-4: To a stirred solution of 3-[(2,6-dioxo-3-piperidyl)oxy]benzenesulfonyl chloride 6 (0.2 g, 659 mol) in acetone (10 mL) and water (10 mL), was added potassium fluoride (77 mg, 1.32 mmol) and stirred at room temperature for 2 hours. After completion, the reaction mixture was concentrated under reduced pressure to afford crude product. The crude product was purified by preparative HPLC [Column: SUNFIRE C18, 19×150 mm, 5 micron, Mobile phase A: 0.1% formic acid in water, Mobile phase B: Acetonitrile, Wavelength: 215 nm]and the fractions were lyophilized to afford 3-[(2,6-dioxo-3-piperidyl)oxy]benzenesulfonyl fluoride (A-1) (30 mg, 16% yield) as an off-white solid. LCMS (ES-): m z 286.18 [M-H]-. H-NMR (400 MHz, DMSO-d6): δ 10.85 (s, 1H), 7.73-7.72 (m, 3H), 7.59-7.56 (m, 1H), 5.53-5.49 (m, 1H), 2.73-2.65 (m, 2H), 2.28-2.25 (m, 2H).

Synthesis A2: Synthesis of 2-(2,6-dioxopiperidin-3-yl)-1,3-dioxoisoindolin-5-yl sulfurofluoridate (A-2)

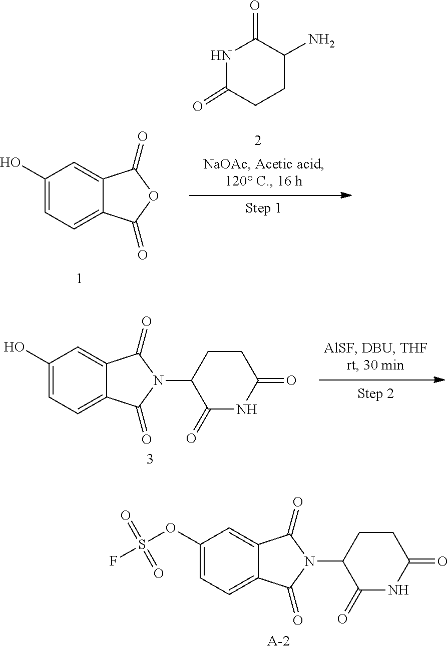

Step-1: To a stirred suspension of 3-aminopiperidine-2,6-dione 1 (586 mg, 4.6 mmol) in acetic acid (10 mL) were added sodium acetate (745 mg, 9.1 mmol, and 491 L), 5-hydroxyisobenzofuran-1,3-dione 2 (0.5 g, 3.05 mmol) and stirred at 120° C. for 16 hours. Upon completion of the reaction, the reaction mixture was quenched with crushed ice and the 10 precipitated solid was collected and dried under reduced pressure to afford 2-(2,6-dioxo-3-piperidyl)-5-hydroxy-isoindoline-1,3-dione 3 (0.5 g, 51% yield) as an off-white solid. LCMS (ES): m/z 275.16 [M+H]*.

Step-2: To a stirred solution of 2-(2,6-dioxo-3-piperidyl)-5-hydroxy-isoindoline-1,3-dione 3 (0.18 g, 657 mol), DBU (250 mg, 1.6 mmol, 245 L) in tetrahydrofuran (6.0 mL) was added (4-acetamidophenyl)(fluorosulfonyl)sulfamoyl fluoride/AISF (247.55 mg, 787.66 mol) and stirred at room temperature for 30 minutes. Upon completion of the reaction, the reaction mixture was concentrated under reduced pressure to afford the crude product. The crude product was purified by preparative HPLC [Column: X-SELECT-C18, 19×250 mm, 5 micron, Mobile phase A: 0.1% Trifluoroacetic acid in water, Mobile phase B: Acetonitrile, Wavelength: 215 nm]and the fractions containing the compound were combined and lyophilized to afford 2-(2,6-dioxo-3-piperidyl)-5-fluorosulfonyloxy-1,3-dioxo-isoindoline (A-2) (36 mg, 15% yield) as an off-white solid. LCMS (ES-): m z 355.14 [M-H]-. H-NMR (400 MHz, DMSO-d6): δ 10.96 (bs, 1H), 8.30 (bs, 1H), 8.18-8.12 (m, 2H), 5.22 (q, J=5.6 Hz, 1H), 2.94-2.86 (m, 1H), 2.63-2.53 (bs, 2H), 2.09 (t, J=6.8 Hz, 1H).

Synthesis A3: Synthesis of 2-(2,6-dioxopiperidin-3-yl)-1,3-dioxoisoindoline-5-sulfonyl 5 fluoride (A-3)

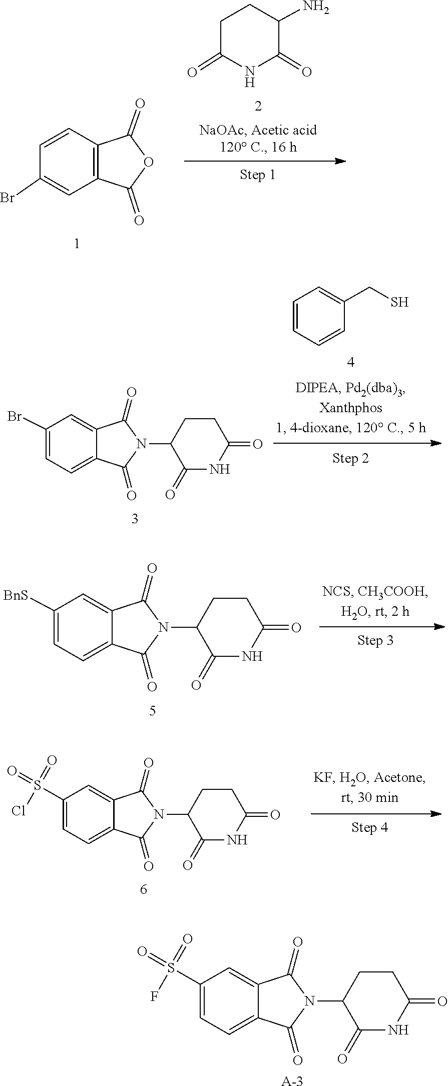

Step-i: To a stirred suspension of 3-aminopiperidine-2,6-dione 1 (847 mg, 6.6 mmol) in acetic acid (15 mL) were added sodium acetate (1.1 g, 13.2 mmol) and 5-bromoisobenzofuran-1,3-dione 2 (1 g, 4.4 mmol) and stirred at 120° C. for 16 hours. Upon completion of the reaction, the reaction mixture quenched with crushed ice and the precipitated solid was collected and 15 dried under reduced pressure to afford 5-bromo-2-(2,6-dioxo-3-piperidyl)isoindoline-1,3-dione 3 (1 g, 52% yield) as a brown solid. LCMS (ES*): m z 338.80 [M+H]+. Step-2: To a stirred solution of 5-bromo-2-(2,6-dioxo-3-piperidyl)isoindoline-1,3-dione 3 (0.5 g, 1.5 mmol) and phenylmethanethiol 4 (368 mg, 3.0 mmol) in dioxane (10 mL) was added N,N-diisopropylethylamine (575 mg, 4.5 mmol, 775 L) and the reaction mixture was purged with argon gas for 10 minutes. After, Pd2(dba)3 (136 mg, 148.3 mol) and Xanthphos (172 mg, 297 mol) were added, and the reaction mixture was stirred at 120° C. for 5 hours. Upon completion of reaction, the reaction mixture was filtered through a celite bed and washed with ethyl acetate (20 mL). The filtrate was concentrated under reduced pressure to afford the crude product, which was purified by column chromatography (Davisil silica, 90% ethyl acetate in 10 petroleum ether as an eluent) to afford 5-benzylsulfanyl-2-(2,6-dioxo-3-piperidyl)isoindoline-1,3-dione 5 (550 mg, 94% yield) as a pale-yellow solid. LCMS (ES*): m z 381.24 [M+H]*.

Step-3: To a stirred solution of 5-benzylsulfanyl-2-(2,6-dioxo-3-piperidyl)isoindoline-1,3-dione 5 (0.2 g, 525.74 mol) in acetic acid (9 mL) and water (1 mL) was added 1-chloropyrrolidine-2,5-dione (210.61 mg, 1.58 mmol) at 0° C. and stirred for 2 hours at room temperature. Upon completion, the reaction was quenched with saturated sodium bicarbonate solution (10 mL) and extracted with ethyl acetate (2×10 mL). The combined organic layer was dried over sodium sulfate and concentrated under reduced pressure to obtain crude product 2-(2,6-dioxo-3-piperidyl)-1,3-dioxo-isoindoline-5-sulfonyl chloride 6 (0.18 g, 71% yield) as an off-white solid without further purification. LCMS (ES+): m z 357.18 [M+H]*.

Step-4: To a stirred solution of 2-(2,6-dioxo-3-piperidyl)-1,3-dioxo-isoindoline-5-sulfonyl chloride 6 (0.2 g, 561 mol) in water (10 mL) and acetone (10 mL) was added potassium fluoride (65 mg, 1.1 mmol) at room temperature. The reaction mixture was stirred for 30 25 minutes at room temperature. After completion of reaction, the reaction mixture was concentrated under reduced pressure to afford the crude product. The residue obtained was purified by preparative HPLC [Column: X-SELECT-C18, 19×250 mm, 5 micron, Mobile phase A: 0.1% Trifluoroacetic acid in water, Mobile phase B: Acetonitrile, Wavelength: 215 nm]and the fractions containing the compound were combined and lyophilized to afford 2-(2,6-dioxo-3-piperidyl)-1,3-dioxo-isoindoline-5-sulfonyl fluoride (A-3) (17 mg, 8% yield) as off-white solid. LCMS (ES-): m z 339.14 [M-H]-. H-NMR (400 MHz, DMSO-d6): δ 11.18 (s, 1H), 8.67-8.59 (m, 2H), 8.29 (d, J=8 Hz, 1H), 5.28 (q, J=5.6 Hz, 1H), 2.95-2.85 (m, 1H), 2.70-2.60 (m, 2H), 2.15-2.10 (m, 1H).

Synthesis A4: Synthesis of 2-(2,6-dioxopiperidin-3-yl)-3-oxoisoindoline-5-sulfonyl fluoride (A-4)

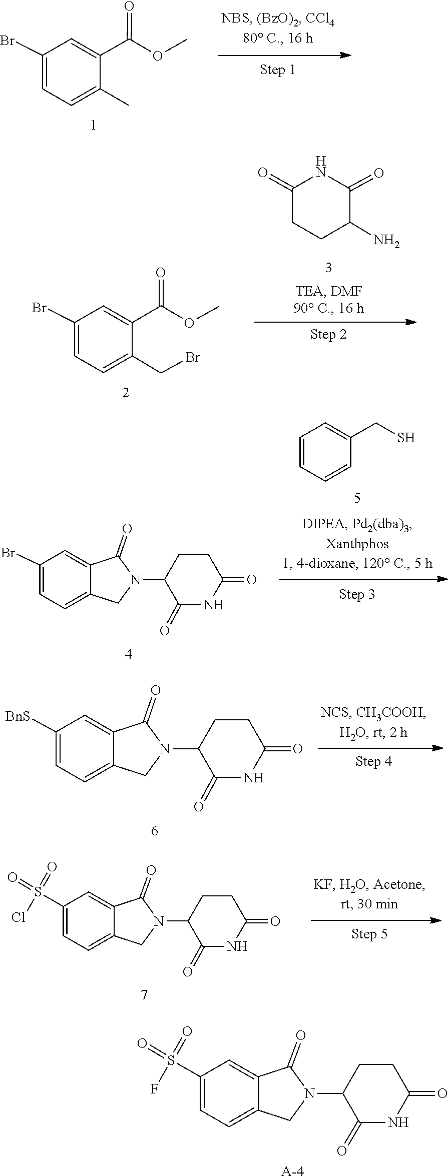

Step-1: To a stirred solution of methyl 5-bromo-2-methyl-benzoate 1 (1 g, 4.4 mmol) in CCl4 (50 mL) were added N-bromosuccinimide (855 mg, 4.8 mmol), benzoyl peroxide (106 mg, 437 mol) and stirred at 80° C. for 16 hours. Upon completion of reaction, the reaction mixture was filtered through a celite bed and washed with ethyl acetate (2×20 mL). The filtrate was concentrated under reduced pressure to afford the crude product, which was purified by column chromatography (Davisil silica, 2% ethyl acetate in petroleum ether as an eluent) to afford methyl 5-bromo-2-(bromomethyl)benzoate 2 (0.8 g, 51% yield) as brown oil. H-NMR (400 MHz, DMSO-d6): δ 7.99 (d, J=2.4 Hz, 1H), 7.87 (q, J=8 Hz, 1H), 7.58 (d, J=8.4 Hz, 1H), 4.98 (s, 2H), 3.88 (s, 3H).

Step-2: To a stirred solution of methyl 5-bromo-2-(bromomethyl)benzoate 2 (0.8 g, 2.6 mmol) in dimethylformamide (8 mL) were added triethylamine (1.3 g, 13 mmol, 1.8 mL), 3-aminopiperidine-2,6-dione 3 (0.5 g, 3.9 mmol) and stirred at 90° C. for 16 hours. Upon completion of the reaction, the reaction mixture was quenched with crushed ice and the precipitated solids were collected and dried under reduced pressure to afford 3-(6-bromo-1-oxo-isoindolin-2-yl) piperidine-2,6-dione 4 (0.26 g, 30% yield) as a pale-violet solid. LCMS (ES*): m z 325.28 [M+H]+.

Step-3: To a stirred solution of 3-(6-bromo-1-oxo-isoindolin-2-yl) piperidine-2,6-dione 4 (0.25 g, 774 mol) and phenylmethanethiol 5 (181.3 L, 1.6 mmol) in dioxane (10.2 mL) was added N,N-diisopropylethylamine (404.26 L, 2.3 mmol) and purged with argon gas for 10 minutes before adding Pd2(dba)3 (71 mg, 77.4 μmol), Xanthphos (90 mg, 155 mol) and stirred at 120° C. for 5 hours. Upon completion of the reaction, the reaction mixture was filtered through a celite bed and washed with ethyl acetate (20 mL). The filtrate was concentrated under reduced pressure to afford the crude product, which was purified by column chromatography (Davisil silica, 90% ethyl acetate in petroleum ether as an eluent) to afford 3-(6-benzylsulfanyl-1-oxo-isoindolin-2-yl) piperidine-2,6-dione 6 (0.2 g, 43% yield) as a pale-yellow solid. LCMS (ES*): m z 367.25 [M+H]+.

Step-4: To a stirred solution of 3-(6-benzylsulfanyl-1-oxo-isoindolin-2-yl) piperidine-2,6-dione 6 (0.2 g, 546 mol) in water (1 mL) and acetic acid (9 mL) was added N-chlorosuccinimide (219 mg, 1.6 mmol) at 0° C. and stirred for 2 hours at room temperature.

Upon completion, the reaction was quenched with saturated sodium bicarbonate solution (10 mL) and extracted with ethyl acetate (2×25 mL), the combined organic layer was dried over sodium sulfate and concentrated under reduced pressure to afford crude product 2-(2,6-dioxo-3-piperidyl)-3-oxo-isoindoline-5-sulfonyl chloride 7 (0.18 g, 44% yield) as an off-white solid.

LCMS (ES*): m z 342.89 [M+H]+.

Step-5: To a stirred solution of 2-(2,6-dioxo-3-piperidyl)-3-oxo-isoindoline-5-sulfonyl chloride 7 (0.18 g, 525.16 mol) in water (10 mL), acetone (10 mL) was added potassium fluoride (61.02 mg, 1.05 mmol) and stirred for 30 minutes at room temperature. Upon completion of reaction, the reaction mixture was concentrated under reduced pressure to afford crude product, which was purified by preparative HPLC [Column: X-SELECT-C18, 19×100 mm, 5 micron, Mobile phase A: 0.1% Formic acid in water, Mobile phase B: Acetonitrile, Wavelength: 215 nm]and the fractions containing the compound were combined and lyophilized to afford 2-(2,6-dioxo-3-piperidyl)-3-oxo-isoindoline-5-sulfonyl fluoride (A-4) (7.7 mg, 4% yield) as an off-white solid. LCMS (ES+): m z 327.24 [M+H]+. H-NMR (400 MHz, DMSO-d6): δ 11.04 (bs, 1H), 8.41 (d, J=8.0 Hz 1H), 8.32 (s, 1H), 8.07 (d, J=8.0 Hz, 1H), 5.20 (q, J=5.2 Hz, 1H), 4.71-4.53 (m, 2H), 2.95-2.85 (m, 1H), 2.70-2.60 (m, 1H), 2.45-2.35 (m, 1H), 2.10-2.00 (m, 1H).

Synthesis A5: Synthesis of 3-((2,6-dioxopiperidin-3-yl)oxy)phenyl sulfurofluoridate (A-5)

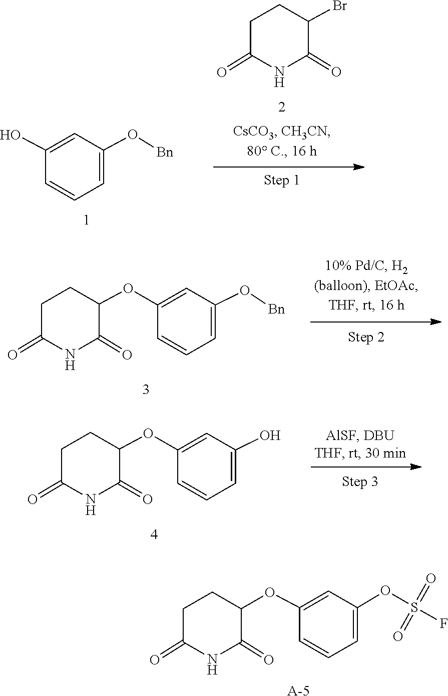

Step-1: To a stirred solution of 3-benzyloxyphenol 1 (1 g, 5.0 mmol) in acetonitrile (6 mL) was 15 added 3-bromopiperidine-2,6-dione 2 (1.9 g, 10.0 mmol), Cs2CO3 (1.63 g, 4.99 mmol) at room temperature and stirred at 80° C. for 16 hours. Upon completion, the reaction mixture was filtered through a celite bed and washed with ethyl acetate (20 mL). The filtrate was concentrated under reduced pressure to afford the crude product, which was purified by column chromatography (Davisil silica, 38% ethyl acetate in petroleum ether as an eluent) to afford 3-(3-benzyloxyphenoxy)piperidine-2,6-dione 3 (0.5 g, 26% yield) as colorless oil. LCMS (ES+): m/z 312.46 [M+H]+.

Step-2: A stirred solution of 3-(3-benzyloxyphenoxy)piperidine-2,6-dione 3 (0.45 g, 1.5 mmol) in tetrahydrofuran (5 mL), ethyl acetate (5 mL) was added 10% Palladium on carbon (769 mg, 7.2 mmol) and the reaction mixture was stirred at room temperature for 16 hours under a hydrogen atmosphere (- 20 psi, balloon pressure). Upon completion of reaction, the reaction mixture was filtered through a celite bed and washed with tetrahydrofuran (25 mL). The filtrate was concentrated under reduced pressure to afford crude 3-(3-hydroxyphenoxy) piperidine-2,6-dione 4 (0.3 g, 71% yield) as a colorless solid. LCMS (ES): m z 222.08 [M+H]*.

Step-3: To a stirred solution of 3-(3-hydroxyphenoxy)piperidine-2,6-dione 4 (0.2 g, 904.12 mol) in tetrahydrofuran (6 mL) was added (4-acetamidophenyl)(fluorosulfonyl)sulfamoyl fluoride/AISF (341 mg, 1.1 mmol) and DBU (338.02 pL, 2.26 mmol) at room temperature.

The reaction mixture was stirred for 30 minutes at room temperature. Upon completion, the reaction mixture was concentrated under reduced pressure to afford crude product. The crude product was purified by preparative HPLC [Column: X-SELECT-C18, 19×150 mm, 5 micron, Mobile phase A: 0.1% trifluoroacetic acid in water, Mobile phase B: Acetonitrile, Wavelength: 215 nm]and the fractions containing the compound were combined and lyophilized to afford 3-(3-fluorosulfonyloxyphenoxy)-2,6-dioxo-piperidine (A-5) (44 mg, 15% yield) as an off-white solid. LCMS (ES-): m z 302.15 [M-H]-. H-NMR (400 MHz, DMSO-d6): δ 10.99 (s, 1H), 20 7.52 (t, J=8.4 Hz 1H), 7.32 (bs, 1H), 7.18 (d, J=2.0 Hz, 2H), 5.36 (q, J=5.6 Hz, 1H), 2.75-2.65 (m, 2H), 2.30-2.20 (m, 2H).

Synthesis A6: Synthesis of 1-(2,6-dioxopiperidin-3-yl)-2-oxo-1,2-dihydrobenzo[cd]indole-4-sulfonyl fluoride (A-6)

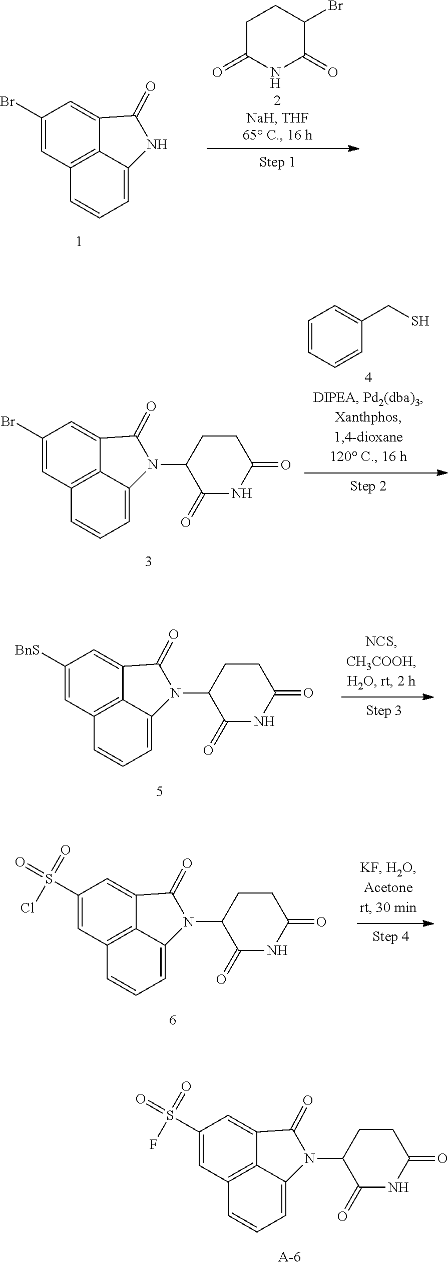

SH DIPEA, Pd₂(dba)₃, BnS NCS, CH₃COOH, Xanthphos, 1, 4-dioxane N O 120° C., 16 h H₂O, rt, 2h NH Step 2 O Step 3 OSP O OI.?O KF, H₂O, Acetone CI N O rt,30 min F N O NH Step4 N 6 A-6

Step-1:

To a stirred solution of 4-bromo-1H-benzo indol-2-one 1 (1 g, 4.0 mmol) in 5 tetrahydrofuran (50 mL) added sodium hydride (967 mg, 40.3 mmol) slowly at 0° C. raised the temperature to 65° C. before adding 3-bromopiperidine-2,6-dione 2 (3.9 g, 20.2 mmol) as a solution of tetrahydrofuran (10 mL) and stirred at 65° C. for 16 hours. Upon completion of the reaction, the reaction mixture was quenched with saturated ammonium chloride solution and extracted with ethyl acetate (2×25 mL). The combined organic layer was dried over anhydrous 10 sodium sulfate, filtered and concentrated under reduced pressure to afford the crude product.

The crude product was triturated with diethyl ether to afford 3-(4-bromo-2-oxo-benzo [cd]indol-1-yl) piperidine-2,6-dione 3 (0.75 g, 36% yield) as a pale green solid. LCMS (ES*): m z 361.09 [M+H]*.

Step-2: To a stirred solution of 3-(4-bromo-2-oxo-benzo [cd]indol-1-yl) piperidine-2,6-dione 3 (0.3 g, 835.3 mol) and phenylmethanethiol 4 (207 mg, 1.7 mmol) in dioxane (6 mL) was added N,N-diisopropylethylamine (436 L, 2.5 mmol) and purged with argon gas for 10 minutes before adding Pd2(dba)3 (97 mg, 167 mol), Xanthphos (77 mg, 83.5 mol) and stirred at 120° C. for 5 hours. Upon completion, the reaction mixture was filtered through celite bed 20 and washed with ethyl acetate (20 mL). The filtrate was concentrated under reduced pressure to afford the crude product, which was purified by column chromatography (Davisil silica, 45% ethyl acetate in petroleum ether as an eluent) to afford 3-(4-benzylsulfanyl-2-oxo-benzo[cd]indol-1-yl) piperidine-2,6-dione 5 (0.3 g, 77% yield) as pale-yellow solid. LCMS (ES+): m z 403.55 [M+H]+.

Step-3: To a stirred solution of 3-(4-benzylsulfanyl-2-oxo-benzo [cd]indol-1-yl) piperidine-2,6-dione 5 (0.2 g, 497 mol) in acetic acid (9 mL) and water (1 mL) was added N-chlorosuccinimide (199 mg, 1.5 mmol) at 0° C. and stirred for 2 hours at room temperature. Upon completion, the reaction was quenched with saturated sodium bicarbonate solution (10 mL) and extracted with ethyl acetate (2×25 mL). The organic layer was dried over sodium sulfate and evaporated under reduced pressure to afford the crude product 1-(2,6-dioxo-3-piperidyl)-2-oxo-benzo[cd]indole-4-sulfonyl chloride 6 (0.18 g, 24.38% yield) as an off-white solid. LCMS (ES*): m z 379.14 [M+H]*.

Step-4: To a stirred solution of 1-(2,6-dioxo-3-piperidyl)-2-oxo-benzo[cd]indole-4-sulfonyl chloride (0.28 g, 739 mol) in water (10 mL) and acetone (10 mL) was added potassium fluoride (86 mg, 1.5 mmol) stirred for 30 minutes at room temperature. Upon completion of the reaction, the reaction mixture was concentrated under reduced pressure to obtain crude product, which was purified by preparative HPLC [Column: X-SELECT-C18, 19×100 mm, 5 micron, Mobile phase A: 0.1% trifluoroacetic acid in water, Mobile phase B: Acetonitrile, Wavelength: 215 nm]and the fractions containing the compound were combined and lyophilized to afford 1-(2,6-dioxo-3-piperidyl)-2-oxo-benzo[cd]indole-4-sulfonyl fluoride (A-6) (31 mg, 11.41% yield) as a pale-yellow solid. LCMS (ES*): m z 363.24 [M+H]+. H-NMR (400 MHz, DMSO-d6): δ 11.18 (s, 1H), 9.25 (s, 1H), 8.60 (s, 1H), 8.0 (d, J=8.8 Hz, 1H), 7.81 (t, J=7.6 Hz, 1H), 7.47 (d, J=7.6 Hz, 1H), 5.529 (m, 1H), 3.00-3.90 (m, 1H), 2.80-2.70 (m, 2H), 2.20-2.10 (m, 1H).

Synthesis A7: Synthesis of 1-(2,6-dioxopiperidin-3-yl)-2-oxo-1,2-dihydrobenzo[cd]indol-4-yl sulfurofluoridate (A-7)

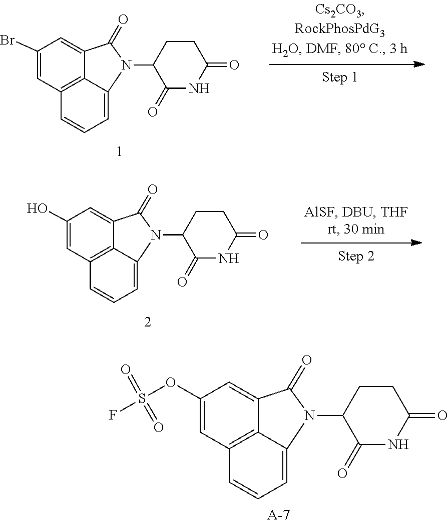

Step-1: To a stirred solution of 3-(4-bromo-2-oxo-benzo[cd]indol-1-yl)piperidine-2,6-dione 1 (0.6 g, 1.7 mmol) in water (30.09 L, 1.67 mmol), dimethylformamide (470 pL) added cesium carbonate (816 mg, 2.5 mmol) and purged with argon gas for 5 minutes before adding RockPhosPdG3 (140 mg, 167.1 mol) and stirred at 80° C. for 3 hours. Upon completion, the reaction mixture was concentrated under reduced pressure to afford the crude product, which was purified by column chromatography (Davisil silica, 70% ethyl acetate in petroleum ether as an eluent) to afford 3-(4-hydroxy-2-oxo-benzo [cd]indol-1-yl) piperidine-2,6-dione 2 (0.1 g, 11% yield) as a pale-yellow solid LCMS (ES-): m z 295.06 [M-H]-.

Step-2: To a stirred solution of 3-(4-hydroxy-2-oxo-benzo[cd]indol-1-yl)piperidine-2,6-dione 2 (0.06 g, 203 mol), 2,3,4,6,7,8,9,10-octahydropyrimido[1,2-a]azepine (76 L, 506 mol) in tetrahydrofuran (3 mL) was added (4-Acetamidophenyl)(fluorosulfonyl)sulfamoyl fluoride/AISF (76 mg, 243 mol) and stirred for 30 minutes at room temperature. Upon completion of the reaction, the reaction mixture was concentrated under reduced pressure to afford crude product, which was purified by preparative HPLC [Column: X-SELECT-C18, 19×150 mm, 5 micron, Mobile phase A: 0.1% trifluoroacetic acid in water, Mobile phase B: Acetonitrile, Wavelength: 215 nm]and the fractions containing the compound were combined and lyophilized to afford 1-(2,6-dioxo-3-piperidyl)-4-fluorosulfonyloxy-2-oxo-benzo[cd]indole (A-7) (11 mg, 14% yield) as a pale-yellow solid. LCMS (ES*): m z 379.24 [M+H]+. ¹H NMR (400 MHz, DMSO-d6) δ: 11.16 (bs, 1H), 8.60 (s, 1H), 8.40 (s, 1H), 7.80 (d, J=8.4 Hz, 1H), 7.70 (t, J=7.2 Hz, 1H), 7.30 (d, J=7.2 Hz, 1H), 5.51 (m, 1H), 3.00-2.90 (m, 1H), 2.85-2.60 (m, 2H), 2.20-2.10 (m, 1H).

Synthesis A8: Synthesis of 2-(2,6-dioxopiperidin-3-yl)-3-oxoisoindolin-5-yl sulfurofluoridate (A-8)

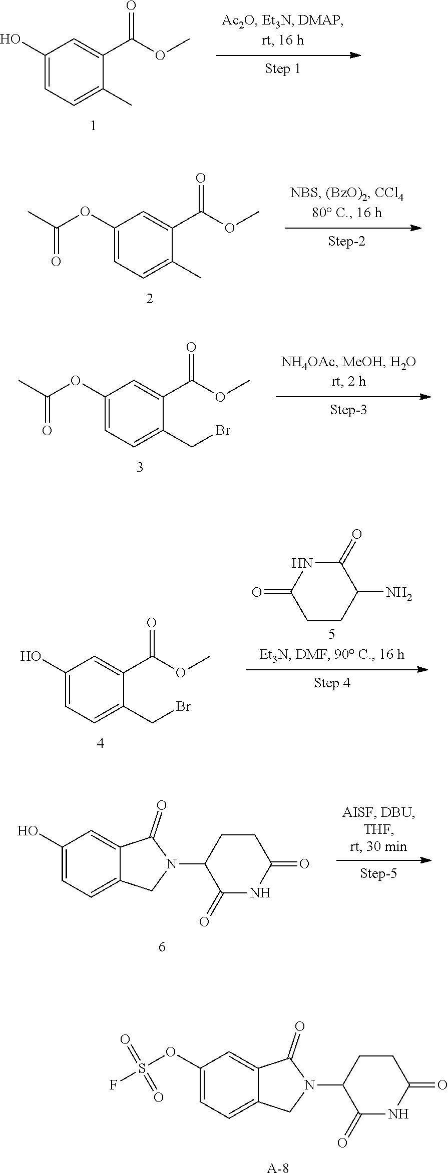

To a stirred solution of methyl 5-hydroxy-2-methyl-benzoate 1 (5 g, 30.1 mmol) in 5 tetrahydrofuran (100 mL) was added N,N-dimethylpyridin-4-amine (368 mg, 3.0 mmol), triethylamine (12.6 mL, 90.27 mmol) and acetic anhydride (4.3 mL, 45.1 mmol) and stirred at room temperature for 16 hours. Upon completion of reaction, the reaction mixture was filtered through a celite bed and washed with ethyl acetate (2×10 mL). The filtrate was concentrated under reduced pressure to obtain the crude product. The crude product was purified by column chromatography (Davisil silica, 2% ethyl acetate in petroleum ether as an eluent) to afford methyl 5-acetoxy-2-methyl-benzoate 2 (6.2 g, 94% yield) as off brown liquid. H-NMR (400 MHz, DMSO-d6): δ 7.56 (d, J=2.4 Hz, 1H), 7.38 (d, J=8.4 Hz, 1H), 7.27 (dd, J=2.8 Hz, 1H), 3.82 (s, 3H), 2.50 (s, 3H), 2.27 (s, 3H).

Step-2: To a stirred solution of methyl 5-acetoxy-2-methyl-benzoate 2 (5 g, 24 mmol) in CCl₄ (250 mL) was added N-bromosuccinimide (4.7 g, 26.4 mmol), benzoyl peroxide (582 mg, 2.4 mmol) and stirred at 80° C. for 16 hours. After completion of the reaction, the reaction mixture was filtered through celite bed and washed with ethyl acetate (2×10 mL). The filtrate was concentrated under reduced pressure to obtain crude product. The crude product was purified by column chromatography (Davisil silica, 23% ethyl acetate in petroleum ether as an eluent) to afford methyl 5-acetoxy-2-(bromomethyl)benzoate 3 (6 g, 70% yield) as light-yellow oil. H-NMR (400 MHz, DMSO-d6): δ 7.70-7.50 (m, 2H), 7.40-7.37 (m, 1H), 5.02 (s, 2H), 3.82 (s, 3H), 2.30 (s, 3H).

Step-3: To a stirred solution of methyl 5-acetoxy-2-(bromomethyl) benzoate 3 (3 g, 10.5 mmol) in water (8 mL) and methanol (30 mL) was added ammonium acetate (18.5 g, 240 mmol) and stirred for 2 h at room temperature. Upon completion, the reaction mixture was quenched with 5 saturated sodium bicarbonate (10 mL) and extracted with ethyl acetate (25 mL). The organic layer was dried over anhydrous sodium sulfate, filtered and concentrated under reduced pressure to afford the crude product, which was purified by column chromatography (Davisil silica, 25% ethyl acetate in petroleum ether as an eluent) to afford methyl 2-(bromomethyl)-5-hydroxy-benzoate 4 (1.2 g, 35% yield) as colorless solid. H-NMR (400 MHz, DMSO-d6): δ 10.14 (bs, 1H), 7.34-7.28 (m, 2H), 6.99 (dd, J=8 Hz, 1H), 5.23 (s, 2H), 3.86 (s, 3H).

Step-4: To a stirred solution of methyl 2-(bromomethyl)-5-hydroxy-benzoate 4 (1.2 g, 4.9 mmol) in dimethylformamide (12 mL) was added triethylamine (3.41 mL, 24.48 mmol) and 3-aminopiperidine-2,6-dione 5 (0.750 g, 5.0 mmol), and stirred for 16 hours at 90° C. Upon 15 completion, the reaction mixture was concentrated under reduced pressure to afford the crude product. The crude product was purified by column chromatography (Davisil silica, 100% ethyl acetate as an eluent) to afford 3-(6-hydroxy-1-oxo-isoindolin-2-yl)piperidine-2,6-dione 6 (0.3 g, 15% yield) as pale-yellow solid. LCMS (ES*): m z 261.10 [M+H]+.

Step-5: To a stirred solution of 3-(6-hydroxy-1-oxo-isoindolin-2-yl)piperidine-2,6-dione 6 (0.05 g, 192 mol) in tetrahydrofuran (2 mL) was added (4-Acetamidophenyl)(fluorosulfonyl) sulfamoyl fluoride/AISF (73 mg, 231 mol) and 2,3,4,6,7,8,9,10-octahydropyrimido[1,2-a]azepine (72 L 480 mol) and stirred at room temperature for 30 minutes. Upon completion of reaction, the reaction mixture was concentrated under reduced pressure to get crude product.

The crude product obtained was purified by preparative HPLC [Column: X-SELECT-C18, 19 x 150 mm, 5 micron, Mobile phase A: 0.1% Formic acid in water, Mobile phase B: Acetonitrile, Wavelength: 215 nm]and the fractions containing the compound were combined and lyophilized to afford 2-(2,6-dioxo-3-piperidyl)-6-fluorosulfonyloxy-1-oxo-isoindoline (A-8) (7.7 mg, 11% yield) as an off-white solid. LCMS (ES*): m z 343.28 [M+H]+. H-NMR (400 MHz, DMSO-d6): δ 11.04 (bs, 1H), 8.00-7.95 (m, 1H), 7.90-7.80 (m, 2H), 5.15 (q, J=8.0 Hz, 1H), 4.60 (dd, J=17.6 Hz, 2H), 2.95-2.85 (m, 1H), 2.70-2.60 (m, 1H), 2.45-2.35 (m, 1H), 2.10-2.00 (m, 1H).

Synthesis A9: Synthesis of 6-((2,6-dioxopiperidin-3-yl)oxy)pyridine-3-sulfonyl fluoride (A-9)

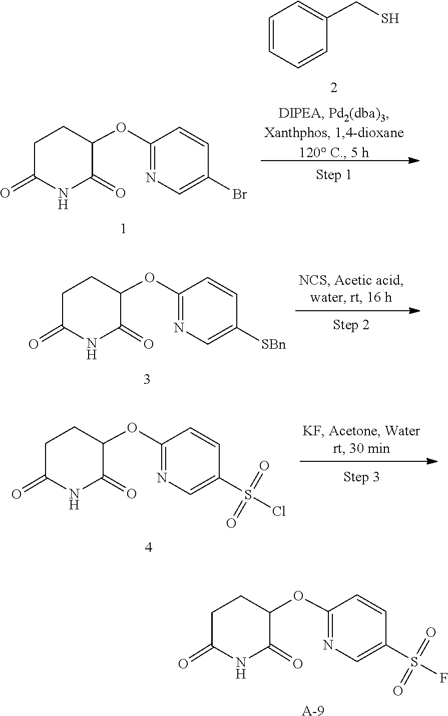

Step-1: To a stirred solution of 3-[(5-bromo-2-pyridyl)oxy]piperidine-2,6-dione 1 (0.5 g, 1.8 mmol) and phenylmethanethiol 2 (436 mg, 3.51 mmol) in dioxane (10 mL) was added N,N-diisopropylethylamine (916 L, 5.3 mmol) and degassed with argon gas for 10 minutes before adding Pd2(dba)3 (203 mg, 351 mol), Xanthphos (161 mg, 175 mol) and stirred at 120° C. for 5 hours. Upon completion of the reaction, the reaction mixture was filtered through celite bed and washed with ethyl acetate (2×20 mL). The filtrate was concentrated under reduced pressure to afford the crude product, which was purified by column chromatography chromatography (Davisil silica, 90% ethyl acetate in petroleum ether as an eluent) to afford 3-[(5-benzylsulfanyl-2-pyridyl)oxy]piperidine-2,6-dione 3 (0.5 g, 85% yield) as a brown solid.

LCMS (ES+): m z 329.17 [M+H]+.

Step-2: To a stirred solution of 3-[(5-benzylsulfanyl-2-pyridyl)oxy]piperidine-2,6-dione 3 (0.1 g, 305 mol) in water (1 mL), acetic acid (4 mL) was added N-chlorosuccinimide (122 mg, 914 mol) at 0° C. and stirred at room temperature for 2 hours. Upon completion of the reaction the reaction mixture was quenched with water and diluted with saturated sodium bicarbonate solution (20 mL) and extracted with ethyl acetate (2×20 mL). The combined organic layer was dried over sodium sulfate and concentrated under reduced pressure to obtain the product 6-[(2,6-dioxo-3-piperidyl) oxy]pyridine-3-sulfonyl chloride 4 (0.09 g, 60% yield) was off white solid. LCMS (ES*): m z 305.05 [M+H]+.

Step-3: To a stirred solution of 6-[(2,6-dioxo-3-piperidyl)oxy]pyridine-3-sulfonyl chloride 4 5 (0.1 g, 328 mol) in water (5 mL), acetone (5 mL) was added potassium fluoride (38 mg, 656 mol) and stirred at room temperature for 30 minutes. After completion of the reaction, the reaction mixture was concentrated under reduced pressure to obtain crude product, which was purified preparative HPLC [Column: X-SELECT-C18, 19×250 mm, 5 micron, Mobile phase A: 0.1% Formic acid in water, Mobile phase B: Acetonitrile, Wavelength: 215 nm]and the 10 fractions containing the compound were combined and lyophilized to afford 6-[(2,6-dioxo-3-piperidyl)oxy]pyridine-3-sulfonyl fluoride (A-9) (26.5 mg, 26% yield) as an off-white solid.

LCMS (ES-): m z 287.17 [M-H]-.¹H NMR (400 MHz, DMSO-d6) δ: 11.03 (s, 1H), 8.92 (bs, 1H), 8.47 (dd, J=2.4 Hz, 1H), 7.27 (d, J=8.8 Hz, 1H) 6.127 (q, J=6.8 Hz, 1H), 2.90-2.80 (m, 1H), 2.70-2.60 (m, 1H), 2.35-2.20 (m, 2H).

Synthesis A10: Synthesis of 3-(2,4-dioxotetrahydropyrimidin-1(2H)-yl)-1-methyl-1H-indazole-7-sulfonyl fluoride (A-10)

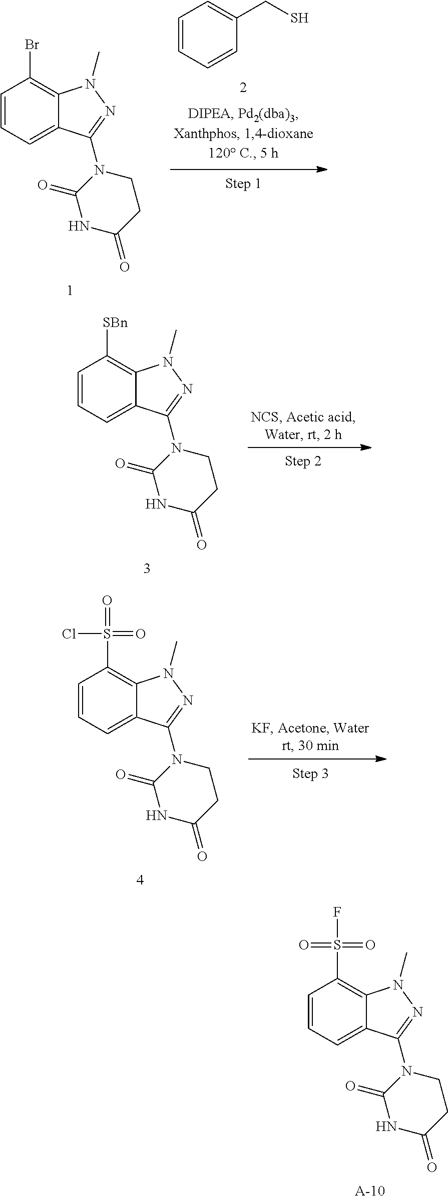

20 Step-1 To a stirred solution of 1-(7-bromo-1-methyl-indazol-3-yl)hexahydropyrimidine-2,4-dione 1 (500 mg, 1.6 mmol) and phenylmethanethiol 2 (384 mg, 3.1 mmol) in dioxane (1.2 mL) added N,N-diisopropylethylamine (809 pL, 4.6 mmol) and purged with argon gas for 10 minutes before adding tris(dibenzylideneacetone)dipalladium(0) (142 mg, 155 mol), Xanthphos (179 mg, 309 μmol) and stirred at 120° C. for 5 hours. Upon completion of reaction, the reaction mixture was filtered through celite bed and washed with ethyl acetate (20 mL). The filtrate was concentrated under reduced pressure to afford the crude product, which was purified by column chromatography (Davisil silica, 35% ethyl acetate in petroleum ether as an eluent) to afford 1-(7-benzylsulfanyl-1-methyl-indazol-3-yl)hexahydropyrimidine-2,4-dione 3 (300 mg, 815 mol, 53% yield, No Salt) as an off-white solid. LCMS (ES*): m z 366.91 [M+H]+.

Step-2: To a stirred solution of 1-(7-benzylsulfanyl-1-methyl-indazol-3-yl) hexahydropyrimidine-2, 4-dione 3 (150 mg, 409 mol) in acetic acid (8 mL), water (1 mL) added N-chlorosuccinimide (55 mg, 409 mol) and stirred at room temperature for 2 hours.

Upon completion of reaction, the reaction mixture was diluted with water (10 mL) and extracted with ethyl acetate (2×25 mL). The combined organic layer was dried over anhydrous sodium sulfate and evaporated under reduced pressure to obtain 3-(2, 4-dioxohexahydropyrimidin-1-yl)-1-methyl-indazole-7-sulfonyl chloride 4 (150 mg, 51% yield, No Salt). LCMS (ES+): m z 343.25 [M+H]+.

Step-3: To a stirred solution of 3-(2,4-dioxohexahydropyrimidin-1-yl)-1-methyl-indazole-7-sulfonyl chloride 4 (150 mg, 438 mol) in acetone (6 mL), water (6 mL) was added potassium fluoride (51 mg, 875 mol) and stirred at room temperature for 1 hour. Upon completion of the reaction, the reaction mixture was concentrated under reduced pressure to obtain crude product, which was purified by preparative HPLC [Column: X-Bridge-C8, 19×150 mm, 5 micron, Mobile phase A: 0.1% Formic acid in water, Mobile phase B: Acetonitrile, Wavelength: 215 nm]and the fractions containing the compound were combined and lyophilized to afford desired product 3-(2,4-dioxohexahydropyrimidin-1-yl)-1-methyl-indazole-7-sulfonyl fluoride (A-10) (45 mg, 31% yield, No salt) as an off-white solid. LCMS (ES*): m z 327.28 [M+H]+. H-NMR (400 MHz, DMSO-d6): δ 10.71 (s, 1H), 8.31 (q, J=8.00 Hz, 2H), 7.46 (t, J=8.00 Hz, 1H), 4.24 (s, 3H), 3.98 (t, J=6.40 Hz, 2H), 2.79 (t, J=6.80 Hz, 2H). Synthesis All: Synthesis of 3-((2,6-dioxopiperidin-3-yl)(methyl)amino)benzenesulfonyl fluoride (A-11)

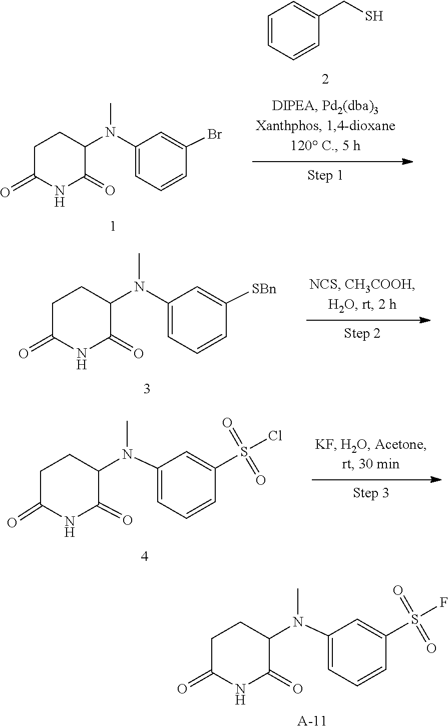

5 Step-1: To a stirred solution of 3-(3-bromo-N-methyl-anilino)piperidine-2,6-dione 1 (300 mg, 1.0 mmol), phenylmethanethiol 2 (251 mg, 2.0 mmol) in dioxane (10 mL) added N,N-diisopropylethylamine (528 L, 3.0 mmol) and purged with argon for 5 minutes before adding tris(dibenzylideneacetone)dipalladium (0) (93 mg, 101 mol), Xanthphos (117 mg, 202 mol) 10 and stirred at 120° C. for 5 hours. Upon completion of the reaction, the reaction mixture was filtered under celite bed and concentrated under reduced pressure to get crude product, which was purified by flash column (Davisil silica, 55% ethyl acetate in petroleum ether as an eluent) to afford 3-(3-benzylsulfanyl-N-methyl-anilino)piperidine-2,6-dione 3 (250 mg, 72% yield).

LCMS (ES*): m/z 341.36 [M+H]+. 15 Step-2: To a stirred solution of 3-(3-benzylsulfanyl-N-methyl-anilino) piperidine-2,6-dione 3 (150 mg, 441 mol) in acetic acid (8 mL), water (1 mL) was added N-chlorosuccinimide (177 mg, 1.3 mmol) at 0° C. stirred at room temperature for 2 hours. Upon completion of the reaction, the reaction mixture was diluted with water (10 mL) and extracted with ethyl acetate (2×25 20 mL). The organic layer was washed with saturated sodium chloride solution (10 mL), dried over anhydrous sodium sulfate, and evaporated under reduced pressure to afford crude product 3-[(2,6-dioxo-3-piperidyl)-methyl-amino]benzenesulfonyl chloride 4 (150 mg, 39.77% yield).

LCMS (ES*): m/z 317.11 [M+H]+.

Step-3: To a stirred solution of 3-[(2,6-dioxo-3-piperidyl)-methyl-amino]benzenesulfonyl chloride 4 (150 mg, 474 mol) in acetone (6 mL), water (6 mL) was added potassium fluoride (55 mg, 947 mol) and stirred at room temperature for 1 hour. Upon completion of reaction, the reaction mixture was concentrated under reduced pressure to obtain crude product which was purified by preparative HPLC [Column: X-Select-C8, 19×250 mm, 5 micron, Mobile phase A: 0.1% Trifluoroacetic acid in water, Mobile phase B: Acetonitrile, Wavelength: 215 nm]and the fractions containing the compound were combined and lyophilized to afford desired product 3-[(2,6-dioxo-3-piperidyl)-methyl-amino]benzenesulfonyl fluoride (A-11) (14.9 mg, 10% yield) as an off-white solid. LCMS (ES*): m z 301.31 [M+H]+. H-NMR (400 MHz, DMSO-d6): δ 10.89 (s, 1H), 7.55 (t, J=8.00 Hz, 1H), 7.32-7.34 (m, 3H), 5.09 (dd, J=4.80, 12.40 Hz, 1H), 2.7-2.95 (m, 4H), 2.5-2.6 (m, 1H), 2.3-2.5 (m, 1H), 1.8-2.0 (m, 1H).

Synthesis A12: Synthesis of 4-(2,6-dioxopiperidin-3-yl)benzenesulfonyl fluoride (A-12)

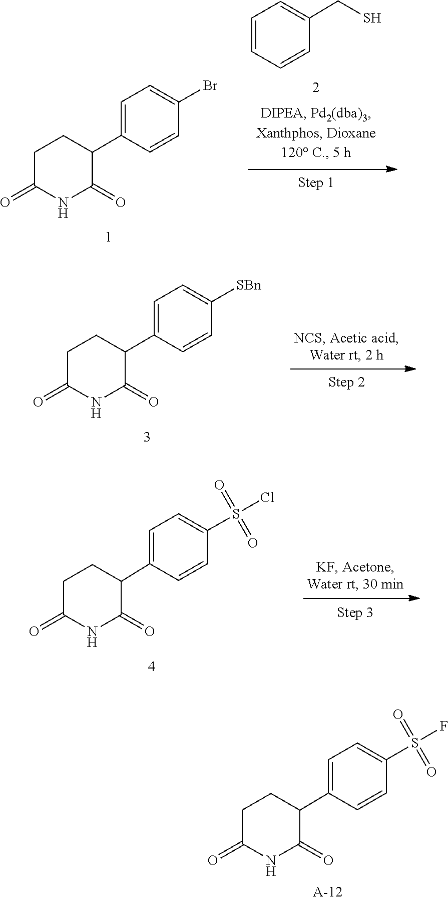

Step-1: To a stirred solution of 3-(4-bromophenyl) piperidine-2,6-dione 1 (0.4 g, 1.5 mmol), phenylmethanethiol 2 (371 mg, 3.0 mmol) in dioxane (10.2 mL) added N,N-diisopropylethylamine (780 L, 4.5 mmol) and degassed with argon gas for 10 minutes before adding Pd2(dba)3(137 mg, 14 mol), Xanthphos (173 mg, 298 mol) and stirred at 120° C. for 5 hours. Upon completion of reaction, the reaction mixture was filtered through a celite bed and washed with ethyl acetate (2×20 mL). The filtrate was concentrated under reduced pressure to obtain crude product, which was purified by column chromatography column (Davisil silica, 100% ethyl acetate as an eluent) to afford 3-(4-benzylsulfanylphenyl) piperidine-2,6-dione 3 (0.3 g, 62% yield) as pale-yellow solid. LCMS (ES*): m z 312.20 [M+H]+.

Step-2: To a stirred solution of 3-(4-benzylsulfanylphenyl) piperidine-2,6-dione 3 (0.15 g, 481.7 mol) in water (2 mL), acetic acid (7 mL) added N-chlorosuccinimide (193 mg, 1.5 mmol) and stirred at room temperature for 2 hours. Upon completion of the reaction, the reaction mixture was diluted with water (10 mL) and extracted with ethyl acetate (2×25 mL).

The combined organic layer was dried over sodium sulfate and evaporated under reduced pressure to obtain the crude product 4-(2,6-dioxo-3-piperidyl) benzenesulfonyl chloride 4 (0.12 g, 61% yield) as an off-white solid. LCMS (ES-): m z 285.96 [M-H]-.

Step-3: To a stirred solution of 4-(2,6-dioxo-3-piperidyl) benzenesulfonyl chloride 4 (0.12 g, 417 mol) in water (7 mL), acetone (7 mL) was added potassium fluoride (48 mg, 83 mol) and stirred at room temperature for 30 minutes. Upon completion of the reaction, the reaction mixture was concentrated under reduced pressure to obtain crude product, which was purified by preparative HPLC [Column: X-Select-C8, 19×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 4-(2,6-dioxo-3-piperidyl)benzenesulfonyl fluoride (A-12) (22.5 mg, 81.35 mol, 19.51% yield, No salt) as an off-white solid. LCMS (ES-): m z 270.17 [M-H]-.¹H-NMR (400 MHz, DMSO-d6): δ 10.97 (s, 1H), 8.12 (d, J=8.4 Hz, 2H), 7.69 (d, J=8.4 Hz, 2H), 4.17 (dd, J=4.8 Hz, 1H), 2.80-2.65 (m, 1H), 2.60-2.55 (m, 1H), 2.35-2.25 (m, 1H), 2.10-2.00 (m, 1H).

Synthesis A13: Synthesis of 5-((2,6-dioxopiperidin-3-yl)oxy)-2-methylbenzenesulfonyl 25 fluoride (A-13)

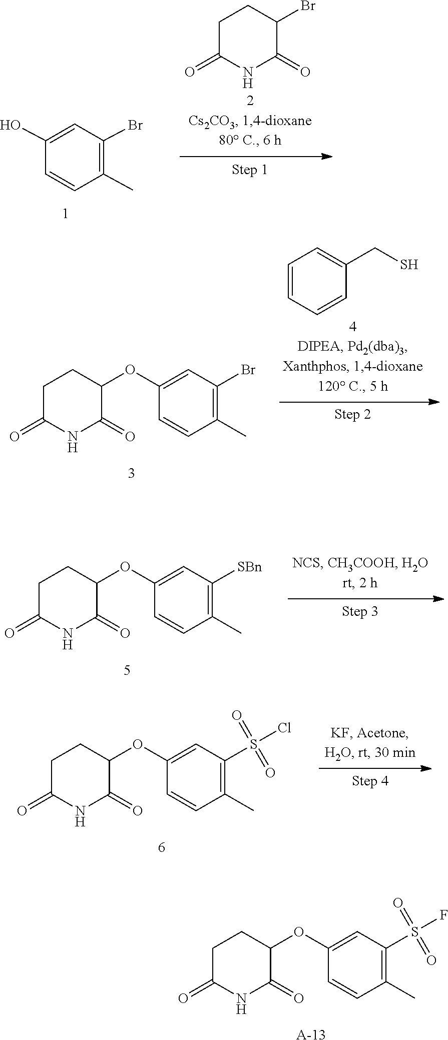

To a stirred solution of 3-bromo-4-methyl-phenol 1 (1.5 g, 8.0 mmol) in dioxane (15 mL) was added Cs2CO3 (7.8 g, 24.1 mmol), 3-bromopiperidine-2,6-dione 2 (1.9 g, 9.6 mmol) and stirred at 80° C. for 6 hours. Upon completion of the reaction, the reaction mixture was filtered over a celite bed and washed with ethyl acetate (2×20 mL). The filtrate was evaporated under reduced pressure to obtain the crude product, which was purified by flash column chromatography (Davisil silica, 10% ethyl acetate in petroleum ether as an eluent) to afford 3-(3-bromo-4-methyl-phenoxy)piperidine-2,6-dione 3 (0.88 g, 36% yield) as colorless viscous oil. LCMS (ES*): m z 297.987 [M+H]+.

Step-²: To a stirred solution of 3-(3-bromo-4-methyl-phenoxy)piperidine-2,6-dione 3 (0.5 g, 1.7 mmol) and phenylmethanethiol 4 (417 mg, 3.4 mmol) in dioxane (10 mL) added N,N-diisopropylethylamine (876.36 L, 5.03 mmol) and purged with nitrogen for 10 minutes before adding Pd2(dab)3 (154 mg, 167.7 mol), Xanthphos (194 mg, 335.4 mol) and stirred at 120° C. for 5 hours. Upon completion of the reaction, the reaction mixture was filtered through a celite bed and washed with ethyl acetate (2×20 mL). The filtrate was concentrated under reduced pressure to obtain the crude product, which was purified by flash column chromatography (Davisil silica, 40% ethyl acetate in petroleum ether as an eluent) to afford 3-(3-benzylsulfanyl-4-methyl-phenoxy)piperidine-2,6-dione 5 (0.21 g, 20% yield) as a pale-yellow solid. LCMS (ESI): m/z 340.12 [M-H]-.

Step-³: To a stirred solution of 3-(3-benzylsulfanyl-4-methyl-phenoxy)piperidine-2,6-dione 5 (0.21 g, 615 mol) in water (2 mL), CH3COOH (10 mL) was added N-chlorosuccinimide (164.26 mg, 1.23 mmol) and stirred at room temperature for 2 hours. Upon completion of the reaction, the reaction mixture was diluted with water (10 mL) and extracted with ethyl acetate (25 mL×2). The combined organic layer was dried over sodium sulfate and concentrated under reduced pressure to afford crude product 5-[(2,6-dioxo-3-piperidyl)oxy]-2-methyl-benzenesulfonyl chloride 6 (0.2 g, 63% yield) as an off-white solid LCMS (ES-): m z 340.12 [M-H]-.

Step-4: To a stirred solution of 5-[(2,6-dioxo-3-piperidyl)oxy]-2-methyl-benzenesulfonyl chloride 6 (0.2 g, 629 mol) in acetone (10 mL), water (10 mL) was added potassium fluoride (73 mg, 1.3 mmol) and stirred at room temperature for 30 minutes. Upon completion of the reaction, the reaction mixture was concentrated under reduced pressure to obtain crude product, which was purified by preparative HPLC [Column: X-BRIDGE-C8, 19×150 mm, 5 micron, Mobile phase A: 0.1% formic acid in water, Mobile phase B: acetonitrile, Wavelength: 215 nm]and the fractions containing the compound were combined and lyophilized to afford the desired product 5-[(2,6-dioxo-3-piperidyl)oxy]-2-methyl-benzenesulfonyl fluoride (A-13) (18.3 mg, 10% yield) as an off-white solid. LCMS (ES*): m z 316.01 [M+H]+.H-NMR (400 MHz, DMSO-d6): δ 10.99 (s, 1H), 7.62 (d, J=2.4 Hz, 1H), 7.57 (d, J=8.8 Hz, 1H), 7.49-7.46 (m, 1H), 5.44-5.39 (m, 1H), 2.72-2.64 (m, 2H), 2.56 (s, 3H), 2.26-2.16 (m, 2H).

Synthesis A14: Synthesis of 4-(2,4-dioxotetrahydropyrimidin-1(2H)-yl)benzenesulfonyl fluoride (A-14)

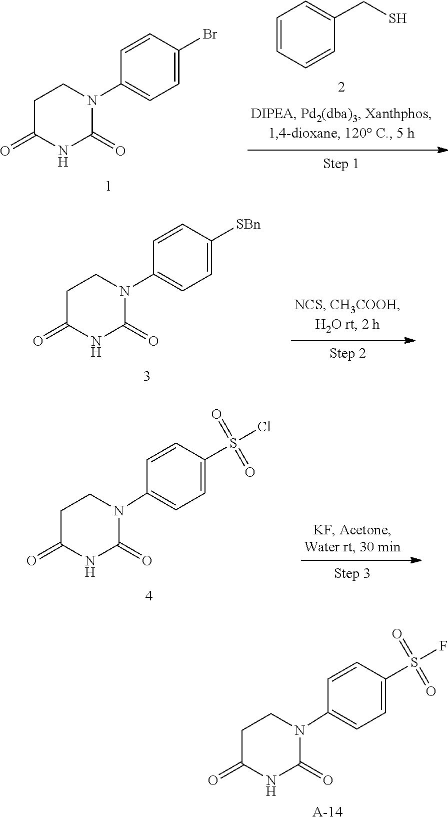

To a stirred solution of 1-(4-bromophenyl)hexahydropyrimidine-2,4-dione 1 (400 mg, 1.5 mmol), phenylmethanethiol 2 (369 mg, 3.0 mmol) in dioxane (10 mL) was added N,N-diisopropylethylamine (776.74 pL, 4.5 mmol) and purged with nitrogen gas for 5 minutes before adding Pd2(dba)3 (136 mg, 149 mol), Xanthphos (172 mg, 297 mol) and stirred at 120° C. for 5 hours. Upon completion of the reaction, the reaction mixture was filtered through a celite bed and washed with ethyl acetate (2×25 mL). The combined organic layer was dried over anhydrous sodium sulfate and concentrated under reduced pressure to obtain 1-(4-benzylsulfanylphenyl) hexahydropyrimidine-2, 4-dione 3 (150 mg, 14% yield) as a clear, colorless oil. LCMS (ES*): m z 313.13 [M+H]+.

Step-2: To a stirred solution of 1-(4-benzylsulfanylphenyl)hexahydropyrimidine-2,4-dione 3 (150 mg, 480 mol) in acetic acid (5.3 mL), water (0.6 mL) was added N-chlorosuccinimide (192 mg, 1.4 mmol) at 0° C. and reaction mixture was stirred at room temperature for 2 hours.

Upon the completion of reaction, the reaction mixture was the reaction was quenched with saturated sodium bicarbonate solution (10 mL) and extracted with ethyl acetate (2×25 mL).

The combined organic layer was dried over anhydrous sodium sulfate and concentrated under reduced pressure to obtain crude product 4-(2, 4-dioxohexahydropyrimidin-1-yl) benzenesulfonyl chloride 4 (150 mg, 5% yield). LCMS (ES-): m z 287.03 [M-H]-.

Step-3: To a stirred solution of 4-(2,4-dioxohexahydropyrimidin-1-yl)benzenesulfonyl chloride 4 (150 mg, 520 mol) in acetone (6 mL), water (6 mL) was added potassium fluoride (60 mg, 1.0 mmol) and stirred at room temperature for 1 hour. Upon completion of the reaction, the reaction mixture was concentrated under reduced pressure to obtain crude product which was purified by preparative HPLC [Column: X-BRIDGE-C8, 19×150 mm, 5 micron, Mobile phase A: 0.1% formic acid in water, Mobile phase B: acetonitrile, Wavelength: 215 nm]and the fractions containing the compound were combined and lyophilized to afford the desired product 4-(2,4-dioxohexahydropyrimidin-1-yl)benzenesulfonyl fluoride (A-14) (5.2 mg, 4% yield). LCMS (ES-): m z 271.56 [M-H]-. ¹H NMR (DMSO-d6 400 MHz): δ 10.64 (s, 1H), 8.14 (d, J=8.80 Hz, 2H), 7.74 (d, J=8.80 Hz, 2H), 3.95 (t, J=6.40 Hz, 2H), 2.75 (t, J=6.40 Hz, 2H).

Synthesis A15: Synthesis of 4-(2,4-dioxotetrahydropyrimidin-1(2H)-yl)phenyl sulfurofluoridate (A-15)

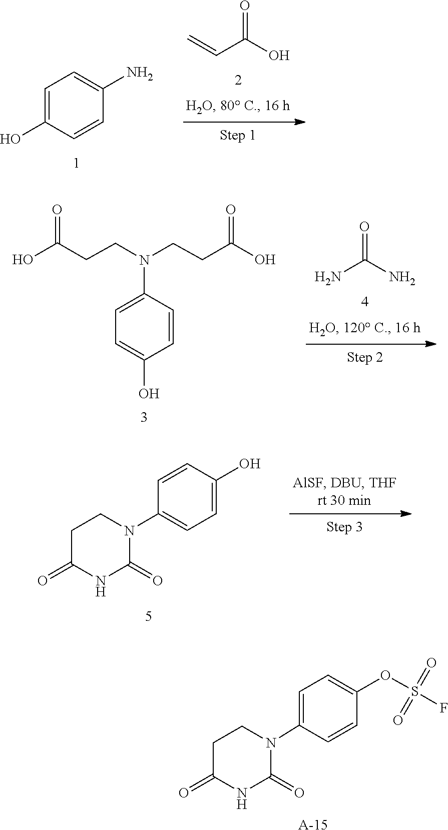

5 Step-1: To a stirred solution of 4-aminophenol 1 (1 g, 9.2 mmol) in water (5 mL) was added acrylic acid 2 (1.4 mL, 20.2 mmol) and stirred at 80° C. for 16 hours. Upon completion of reaction, the reaction was cooled and the precipitated solid was collected by filtration and washed with water (25 mL) and dried under reduced pressure to obtain 3-[N-(2-carboxyethyl)-4-hydroxy-anilino]propanoic acid 3 (1.7 g, 37% yield) as an off-white solid. LCMS (ES*): m z 254.19 [M+H]+.

Step-2: To a solution of 3-[N-(2-carboxyethyl)-4-hydroxy-anilino]propanoic acid 3 (700 mg, 2.8 mmol) in acetic acid (3.5 mL) was added urea 4 (187 pL, 4.2 mmol) and stirred at 120° C. for 12 hours. After being stirred for 12 hours, 1 N HCl (4 mL) was added to the reaction and continued stirring at 120° C. for 1 hour. Upon completion, the reaction mixture was cooled to 0° C. and the precipitated solids were collected by filtration and was washed with water (15 mL), dried under reduced pressure to obtain 1-(4-hydroxyphenyl) hexahydropyrimidine-2, 4-dione 5 (350 mg, 59% yield) as an off-white solid. LCMS (ES+): m z 207.06 [M+H]+.

Step-3: To a stirred solution of 1-(4-hydroxyphenyl)hexahydropyrimidine-2,4-dione 5 (150 mg, 727 mol) in tetrahydrofuran (9.7 mL) was added DBU (272 L, 1.8 mmol,), 4-(acetylamino)phenyl]imidodisulfuryl difluoride/AISF (274 mg, 873 mol) and stirred at room temperature for 30 minutes. Upon completion of the reaction, the reaction mixture was concentrated under reduced pressure to obtain crude product. The residue obtained was purified by preparative HPLC [Column: X-BRIDGE-C8, 19×150 mm, 5 micron, Mobile phase A: 0.1% formic acid in water, Mobile phase B: acetonitrile, Wavelength: 215 nm]and the fractions containing the compound were combined and lyophilized to afford the desired product 1-(4-fluorosulfonyloxyphenyl)-2, 4-dioxo-hexahydropyrimidine (A-15) (67.8 mg, 32% yield) as an off-white solid. LCMS (ES*): m z 289.26 [M+H]+.H-NMR (400 MHz, DMSO-d6): δ 10.48 (s, 1H), 7.63 (d, J=9.20 Hz, 2H), 7.54 (d, J=9.20 Hz, 2H), 3.84 (t, J=6.80 Hz, 2H), 2.72 (t, J=6.40 Hz, 2H).

Synthesis A16: Synthesis of 5-((2,6-dioxopiperidin-3-yl)oxy)-2-methoxybenzenesulfonyl 10 fluoride (A-16)

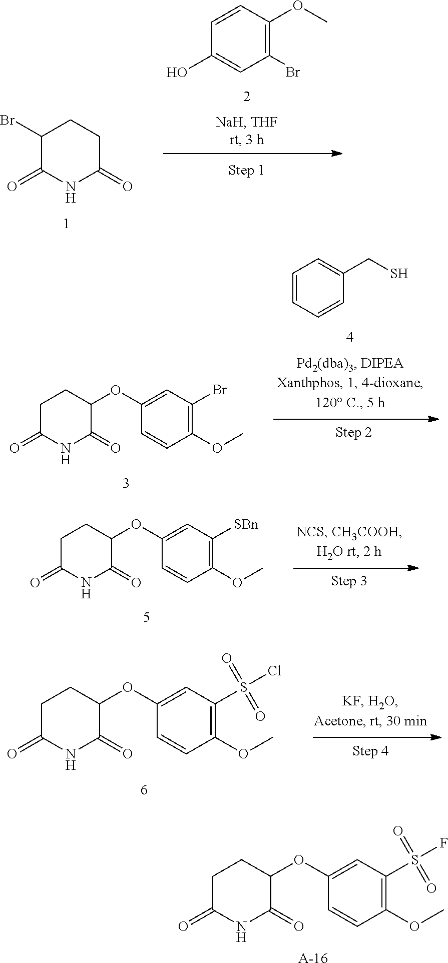

O HO 2 Br Br NaH, THF, O Br rt, 3 h 0 N 011H1% Step I O HH 1 3 4SH Pd₂(dba)₃, DIPEA Xanthphos,1, 4-dioxane, O SBn NCS, CH₃COOH, H₂O 120° C.,5 h rt,2h O N O O 0 Step 2 H Step 3 O S KF, H₂O, Acetone, 0 S-F O rt, 30 min 6 0 N i 0 N o H Step 4 H 0 6 A-16

Step-1: To a solution of 3-bromo-4-methoxy-phenol 2 (1.0 g, 4.9 mmol) in tetrahydrofuran (15 mL) was added NaH (236 mg, 9.9 mmol) at 0° C. and stirred at room temperature for 30 minutes after which was added 3-bromopiperidine-2,6-dione 1 (1.1 g, 5.9 mmol) and stirred at room temperature for 3 hours. Upon completion of the reaction, the reaction mixture was diluted with ice chilled water (50 mL) and extracted ethyl acetate (2×50 ml). The combined organic layer 20 was washed with saturated sodium chloride solution (50 mL), dried over anhydrous sodium sulfate and concentrated under reduced pressure to obtain crude product, which was purified by flash column chromatography (Davisil silica, 30% ethyl acetate in petroleum ether as an eluent) to afford 3-(3-bromo-4-methoxy-phenoxy)piperidine-2,6-dione 3 (0.8 g, 47% yield). H-NMR (400 MHz, DMSO-d6): δ 10.92 (s, 1H), 7.29 (d, J=2.0 Hz, 1H), 7.12-6.95 (m, 2H), 5.15-5.11 (m, 1H), 3.79 (s, 3H), 2.70-2.54 (m, 2H), 2.21-2.05 (s, 2H). Step-2: To a stirred solution of 3-(3-bromo-4-methoxy-phenoxy)piperidine-2,6-dione 3 (527 mg, 1.7 mmol), phenylmethanethiol 4 (417 mg, 3.4 mmol) in dioxane (10 mL) was added N,N-diisopropylethylamine (876 L, 5.0 mmol) and purged with argon gas for 10 minutes before adding Pd2(dba)3 (154 mg, 168 mol), Xanthphos (194 mg, 335 mol) and stirred at 120° C. for 5 hours. Upon completion of reaction, the reaction mixture was filtered through a celite bed and washed with ethyl acetate (20 mL). The filtrate was concentrated under reduced pressure to afford the crude product, which was purified by flash column chromatography (Davisil silica, 40% ethyl acetate in petroleum ether as an eluent) to afford 3-(3-benzylsulfanyl-4-methoxy-phenoxy)piperidine-2,6-dione 5 (450 mg, 402% yield) as a pale-yellow solid. LCMS (ES*): m z 358.17 [M+H]+.

Step-3: To a stirred solution of 3-(3-benzylsulfanyl-4-methoxy-phenoxy)piperidine-2,6-dione 5 (0.4 g, 1.1 mmol) in water (2 mL), acetic acid (10 mL) was added N-chlorosuccinimide (299 mg, 2.2 mmol) at 0° C. and stirred at room temperature for 2 hours. Upon completion of the reaction, the reaction mixture was quenched with saturated sodium bicarbonate solution (15 mL) and extracted with ethyl acetate (2×15 mL). The organic layer was dried over sodium sulfate and concentrated under reduced pressure to afford crude product 5-[(2,6-dioxo-3-piperidyl)oxy]-2-methoxy-benzenesulfonyl chloride 6 (450 mg, 43.81% yield) as an off-white solid. LCMS (ES-): m z 331.73 [M-H]-.

Step-4: To a stirred solution of 5-[(2,6-dioxo-3-piperidyl)oxy]-2-methoxy-benzenesulfonyl chloride 6 (0.25 g, 749 mol) in water (5 mL), acetone (5 mL) was added potassium fluoride (87 mg, 1.5 mmol) stirred at room temperature for 30 minutes. Upon completion of the reaction, the reaction mixture was concentrated under reduced pressure to get crude product, which was purified by preparative HPLC [Column: KINETEX EVO C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 5-[(2,6-dioxo-3-piperidyl)oxy]-2-methoxy-benzenesulfonyl fluoride (A-16) (26 mg, 80.74 mol, 10.78% yield) as an off-white solid. LCMS (ES-): m z 316.14 [M-H]-. H-NMR (400 MHz, DMSO-d6): δ 10.99 (s, 1H), 7.58-7.53 (m, 2H), 7.40 (d, J=9.2 Hz, 1H), 5.32-5.28 (m, 1H), 3.95 (s, 3H), 2.74-2.58 (m, 2H), 2.25-2.23 (s, 2H).

Synthesis A17: Synthesis of 3-((2,6-dioxopiperidin-3-yl)oxy)-5-methylbenzenesulfonyl 5 fluoride (A-17)

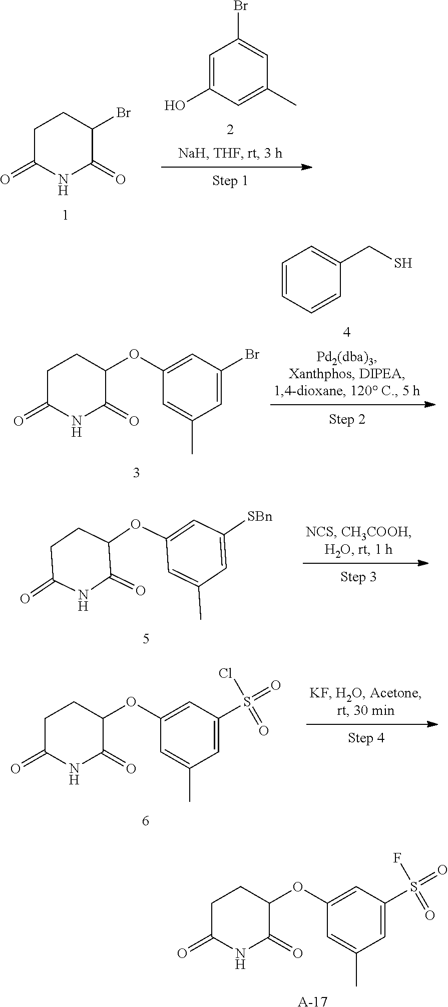

Step-1: To a solution of 3-bromo-5-methyl-phenol 2 (1.0 g, 5.4 mmol) in tetrahydrofuran (10 mL) at 0° C. was added NaH (60% dispersion in oil) (257 mg, 10.7 mmol) and stirred at room temperature for 30 minutes after which was added 3-bromopiperidine-2,6-dione 1 (1.2 g, 6.4 mmol) and stirred at room temperature for 3 hours. Upon completion of the reaction, the reaction mixture was quenched with ice chilled water (50 mL) extracted with ethyl acetate (3 15×50 mL). The combined organic layer was washed with saturated sodium chloride solution (50 ml), dried over anhydrous sodium sulfate and concentrated under reduced pressure to obtain crude product, which was purified by flash column chromatography (Davisil silica, 30% ethyl acetate in petroleum ether as an eluent) afford 3-(3-bromo-5-methyl-phenoxy)piperidine-2,6-dione 3 (0.21 g, 9% yield) as yellow solid. LCMS (ES-):m/z 270.13 [M+H]+. 20 Step-2: To a stirred solution of 3-(3-bromo-5-methyl-phenoxy)piperidine-2,6-dione 3 (0.2 g, 671 mol), phenylmethanethiol 4 (167 mg, 1.3 mmol) in 1,4-dioxane (5 mL) was added N,N-diisopropylethylamine (351 L, 2.0 mmol), xantphos (78 mg, 135 mol) and degassed with argon gas for 10 minutes before adding tris(dibenzylideneacetone)dipalladium(0) (61 mg, 67 mol) and stirred at 120° C. for 5 hours. Upon completion of reaction, the reaction mixture was filtered through celite bed and washed with ethyl acetate (20 mL). The filtrate was concentrated under reduced pressure to afford the crude product, which was purified by flash column chromatography (Davisil silica, 40% ethyl acetate in petroleum ether as an eluent) to afford 3-(3-benzylsulfanyl-5-methyl-phenoxy)piperidine-2,6-dione 5 (0.11 g, 43% yield) as a pale-yellow solid. LCMS (ES*): m z 342.16 [M+H]+.

Step-3: To a stirred solution of 3-(3-benzylsulfanyl-5-methyl-phenoxy)piperidine-2,6-dione 5 (0.11 g, 322 mol) in water (0.8 mL), CH3COOH (3 mL) was added N-chlorosuccinimide (86 mg, 644 mol) at 0° C. and stirred at room temperature for 2 hours. Upon completion of the 15 reaction, the reaction mixture was quenched with water (5 mL) and diluted with ethyl acetate (5 mL) and extracted with saturated. sodium bicarbonate solution (2×10 mL), the organic layer was dried over sodium sulfate and concentrated under reduced pressure to afford the crude product 3-[(2,6-dioxo-3-piperidyl)oxy]-5-methyl-benzenesulfonyl chloride 6 (0.15 g, 81% yield) as an off-white solid. LCMS (ES-): m z 315.96 [M-H]-. 20 Step-4: To a stirred solution of 3-[(2,6-dioxo-3-piperidyl)oxy]-5-methyl-benzenesulfonyl chloride 6 (0.15 g, 472 mol) in water (2 mL) and acetone (2 mL) was added potassium fluoride (55 mg, 944 mol) at room temperature and stirred for 30 minutes. After completion of the reaction, the reaction mixture was concentrated under reduced pressure to afford crude product, which was purified by preparative HPLC [Column: X-BRIDGE C8, 19×250 mm, 5 micron, Mobile phase A: 0.1% Formic acid in water, Mobile phase B: Acetonitrile, Wavelength: 215 nm]and the fractions containing the compound were combined and lyophilized to afford the desired product 3-[(2,6-dioxo-3-piperidyl)oxy]-5-methyl-benzenesulfonyl fluoride (A-17) (17.1 mg, 55.6 mol, 12% yield) as an off-white solid. LCMS (ES-): m z 300.13 [M-H]-. H-NMR (400 MHz, DMSO-d6): δ 10.99 (s, 1H), 7.56-7.53 (d, J=14.0 Hz, 2H), 7.42 (s, 1H), 5.49-5.45 (m, 1H), 2.76-2.64 (m, 2H), 2.42 (s, 3H), 2.25-2.07 (m, 2H). Synthesis A18: Synthesis of 3-((2,6-dioxopiperidin-3-yl)(methyl)amino)phenyl sulfurofluoridate (A-18)

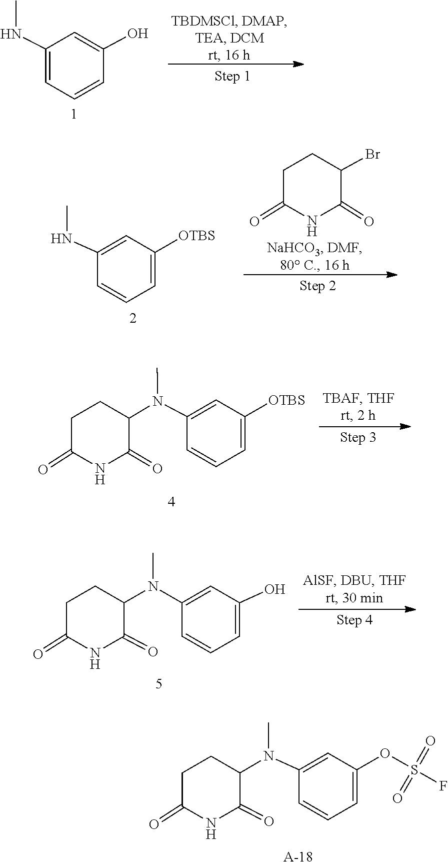

Step-1: To a stirred solution of 3-(methylamino) phenol 1 (1 g, 8.1 mmol) in dichloromethane (10 mL) was added triethylamine (1.70 mL, 12.18 mmol), DMAP (99 mg, 812 mol) before adding TBDMSCI (2.45 g, 16.2 mmol) and stirred at room temperature for 16 hours. Upon 10 completion of the reaction, the reaction mixture was diluted with dichloromethane (25 mL) and water (25 mL) and washed with saturated sodium bicarbonate solution (25 mL). The organic layer was separated, dried over anhydrous sodium sulfate, filtered and concentrated under reduced pressure to obtain crude product 3-[tert-butyl(dimethyl)silyl]oxy-N-methyl-aniline 2 (1.9 g, 94% yield) as brown liquid. LCMS (ES*): m z 237.82 [M+H]+. 15 Step-2: To a stirred solution of 3-[tert-butyl(dimethyl)silyl]oxy-N-methyl-aniline 2 (0.5 g, 2.1 mmol), 3-bromopiperidine-2,6-dione 3 (2.0 g, 10.5 mmol) in dimethylformamide (5 mL) was added sodium bicarbonate (1.42 g, 16.85 mmol) and stirred at 80° C. for 16 hours. Upon completion of reaction, the reaction mixture was diluted with water (50 mL) and extracted with 20 ethyl acetate (2×50 mL). The combined organic layer was dried over anhydrous sodium sulfate, filtered and concentrated under reduced pressure to afford the crude product. The crude product was purified by column chromatography (Davisil silica, 18% ethyl acetate in petroleum ether as an eluent) to afford 3-[3-[tert-butyl(dimethyl)silyl]oxy-N-methyl-anilino]piperidine-2,6-dione 4 (0.15 g, 14% yield) as brown solid. LCMS (ES*): m z 347.15[M+H]+.

Step-3: To a stirred solution of 3-[3-[tert-butyl (dimethyl) silyl]oxy-N-methyl-aniline]piperidine-2,6-dione 4 (0.15 g, 430 mol) in tetrahydrofuran (3 mL) was added tetrabutylammoniumfluoride (860.81 L, 1 M in tetrahydrofuran) at 0° C. and stirred at room temperature for 2 hours. Upon completion of the reaction, the reaction mixture was diluted with water (10 mL) and extracted with ethyl acetate (2×25 mL). The combined organic layer was dried over anhydrous sodium sulfate, filtered and concentrated under reduced pressure to obtain the crude product 3-(3-hydroxy-N-methyl-anilino)piperidine-2,6-dione 5 (0.1 g, 75% yield) as brown solid. LCMS (ES*): m z 235.05 [M+H]+.

Step-4: To a stirred solution of 3-(3-hydroxy-N-methyl-anilino)piperidine-2,6-dione 5 (0.1 g, 427 mol) in tetrahydrofuran (4 mL) was added 2,3,4,6,7,8,9,10-octahydropyrimido[1,2-a]azepine (160 pL, 1.1 mmol), (4-Acetamidophenyl)(fluorosulfonyl)sulfamoyl fluoride/AISF (161 mg, 512 mol) and stirred at room temperature for 30 minutes. Upon completion of reaction, the reaction mixture was concentrated under reduced pressure to obtain crude product, which was purified by preparative HPLC [Column: X-BRIDGE C8, 19×150 mm, 5 micron, Mobile phase A: 0.1% Trifluoroacetic acid in water, Mobile phase B: Acetonitrile, Wavelength: 215 nm]and the fractions containing the compound were combined and lyophilized to afford the desired product 3-(3-fluorosulfonyloxy-N-methyl-anilino)-2,6-dioxo-piperidine (A-18) (14.3 mg, 10% yield) as an off-white solid. LCMS (ES*): m z 317.24 [M+H]+.H-NMR (400 MHz, DMSO-d6): δ 10.86 (s, 1H), 7.35 (t, J=8.4 Hz, 1H), 7.00-6.91 (m, 2H), 6.78 (d, J=8 Hz, 1H), 4.98 (q, J=5.2 Hz, 1H), 2.77 (s, 3H), 2.90-2.80 (m, 1H), 2.58-2.55 (m, 1H), 2.40-2.35 (m, 1H), 1.92-1.89 (m, 1H).

Synthesis A19: Synthesis of 5-((2,6-dioxopiperidin-3-yl)oxy)-2-methylphenyl sulfurofluoridate (A-19)

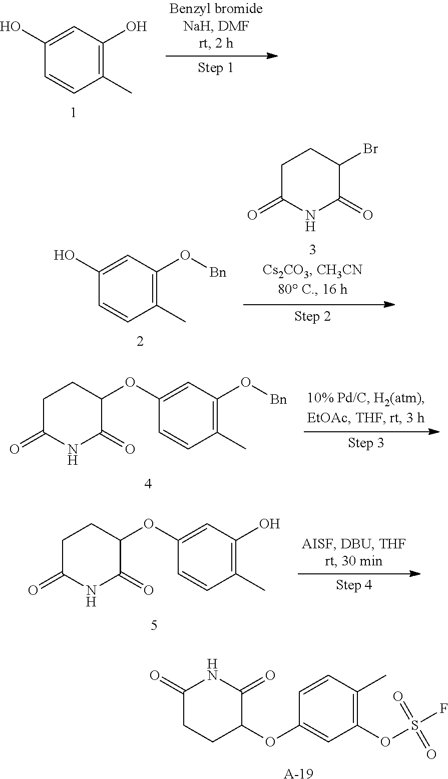

To a stirred solution of 4-methylbenzene-1, 3-diol 1 (2 g, 16 mmol) in 5 dimethylformamide (40 mL) was added NaH (60% dispersion in oil) (773 mg, 32 mmol) at 0° C. before adding benzyl bromide (1.92 mL, 16 mmol) and stirred at room temperature for 2 hours. Upon completion of the reaction, the reaction mixture was quenched with chilled water (10 mL) and extracted with ethyl acetate (2×25 mL). The organic layer was dried over anhydrous sodium sulfate, filtered and concentrated under reduced pressure to obtain crude 10 product. The crude product was purified by column chromatography (60-120 mess silica gel; using 7% ethyl acetate and petroleum ether as an eluent) to afford 3-benzyloxy-4-methyl-phenol 2 (0.68 g, 17% yield) as colorless oil. LCMS (ES-): m z 213.07[M-H]-.

Step-2: To a stirred solution of 3-benzyloxy-4-methyl-phenol 2 (0.4 g, 1.87 mmol) and 3-bromopiperidine-2,6-dione 3 (717 mg, 3.7 mmol) in acetonitrile (6 mL) was added cesium carbonate (1.63 g, 5.0 mmol) and stirred at 80° C. for 16 hours. Upon completion of the reaction, the reaction mixture was filtered through celite bed and washed with ethyl acetate (20 mL).

The filtrate was concentrated under reduced pressure to obtain crude product. The crude product was purified by column chromatography (60-120 mess silica gel; using 38% Ethyl acetate and petroleum ether as an eluent) to afford 3-(3-benzyloxy-4-methyl-phenoxy) piperidine-2,6-dione 4 (0.15 g, 22% yield) as colorless oil. LCMS (ES-): m z 323.86 [M+H]+.

Step-3: To a stirred solution of 3-(3-benzyloxy-4-methyl-phenoxy)piperidine-2,6-dione 4 (0.15 g, 461 mol) in ethyl acetate (3 mL), tetrahydrofuran (3 mL) was purged with nitrogen gas for 25 5 minutes,after which was added 10% Palladium on activated charcoal (24 mg, 2.3 mmol) and stirred under H2 (balloon pressure) at room temperature for 3 hours. Upon completion of the reaction, the reaction mixture was filtered through a celite bed and washed with ethyl acetate (20 mL) and tetrahydrofuran (20 mL). The filtrate was concentrated under reduced pressure to obtain desired product 3-(3-hydroxy-4-methyl-phenoxy) piperidine-2,6-dione 5 (0.1 g, 27% yield) as colorless oil. LCMS (ES-): m z 234.06 [M-H]-.

Step-4: To a stirred solution of 3-(3-hydroxy-4-methyl-phenoxy)piperidine-2,6-dione 5 (0.1 g, 425 mol) in tetrahydrofuran (3 mL) was added 2,3,4,6,7,8,9,10-octahydropyrimido[1,2-a]azepine (158.93 pL, 1.1 mmol) and (4-acetamidophenyl)(fluorosulfonyl)sulfamoyl fluoride/AISF (160 mg, 510 mol) and stirred at room temperature for 30 minutes. Upon completion of the reaction, the reaction mixture was concentrated under reduced pressure to obtain crude product, which was purified by preparative HPLC [Column: X-BRIDGE C8, 19 x 250 mm, 5 micron, Mobile phase A: 0.1% formic acid in water, Mobile phase B: acetonitrile, Wavelength: 215 nm]and the fractions containing the compound were combined and lyophilized to afford the desired product 3-(3-fluorosulfonyloxy-4-methyl-phenoxy)-2,6-dioxo-piperidine (A-19) (15.3 mg, 11% yield) as an off-white solid. LCMS (ES-): m z 316.08 [M-H]-. H-NMR (400 MHz, DMSO-d6): δ 10.97 (s, 1H), 7.39 (d, J=8.4 Hz, 1H), 7.23 (s, 1H), 7.10-7.08 (m, 1H), 5.30 (q, J=5.2 Hz, 1H), 2.71-2.58 (m, 2H), 2.26 (s, 3H), 2.24-2.20 (m, 2H).

Synthesis A20: Synthesis of 3-((2,6-dioxopiperidin-3-yl)oxy)-5-methylphenyl sulfurofluoridate (A-20)

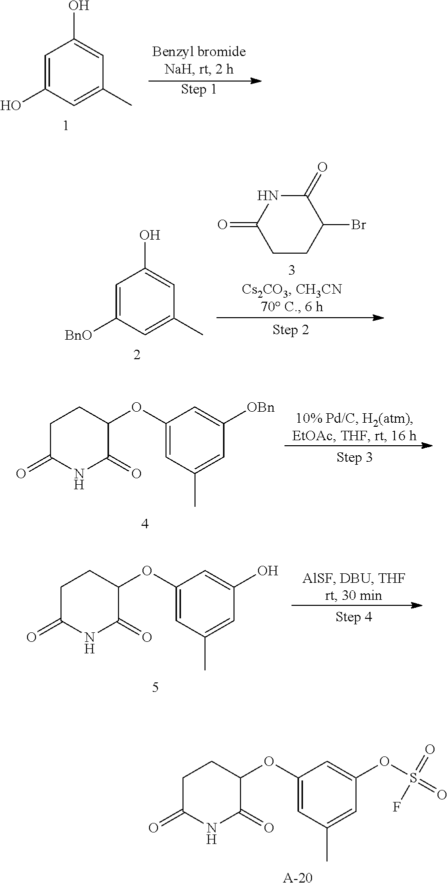

Step-1: To a stirred solution of 5-methylbenzene-1, 3-diol 1 (1 g, 8.1 mmol) in dimethylformamide (5 mL) was added NaH (60% dispersion in oil) (640 mg, 7.8 mmol) and stirred at room temperature for 2 hours. Upon completion of the reaction, the reaction mixture was quenched with chilled water (10 mL) and was extracted with ethyl acetate (2×25 mL).

The organic layer was dried over anhydrous sodium sulfate and concentrated under reduced pressure to get crude product which was then purified by flash column chromatography (60-12 mesh silica gel; using 18% Ethyl acetate in petroleum ether as eluent) to afford desired product 3-benzyloxy-5-methyl-phenol 2 (420 mg, 19% yield) as a thick oil. LCMS (ES-): m z 212.82[M-H]-.

Step-2: To a stirred solution of 3-benzyloxy-5-methyl-phenol 2 (400 mg, 1.9 mmol) in acetonitrile (10 mL) were added cesium carbonate (2.03 g, 3.73 mmol) and 3-bromopiperidine-2,6-dione 3 (430 mg, 2.2 mmol) and stirred the reaction at 70° C. for 3 hours. Upon completion of the reaction, the reaction mixture was filtered and was concentrated under reduced pressure to get crude product, which was then purified by flash column chromatography (60-12 mesh silica gel; using 40% ethyl acetate in petroleum ether as eluent) to afford 3-(3-benzyloxy-5-methyl-phenoxy)piperidine-2,6-dione 4 (130 mg, 18% yield). LCMS (ES*): m z 326.18 [M+H]+.

Step-3: To a stirred solution of 3-(3-benzyloxy-5-methyl-phenoxy) piperidine-2,6-dione 4 (130 mg, 400 mol) in tetrahydrofuran (2 mL), ethyl acetate (2 mL) was purged under nitrogen for 5 minutes after which was added 10% Palladium on carbon (150 mg, 1.4 mmol) and stirred under H2 (30 psi) at room temperature for 3 hours. Upon the completion of the reaction, the reaction mixture was filtered through celite pad and washed with ethyl acetate (25 mL). The filtrate was concentrated under reduced pressure to afford 3-(3-hydroxy-5-methyl-phenoxy) piperidine-2,6-dione 5 (90 mg, 77% yield). LCMS (ES*): m z 235.77 [M+H]+.

Step-4: To a stirred solution of 3-(3-hydroxy-5-methyl-phenoxy)piperidine-2,6-dione 5 (90 mg, 383 mol) in tetrahydrofuran (1 mL) was added DBU (143.04 L, 957 mol) and 4-(acetylamino)phenyl]imidodisulfuryl difluoride/AISF 5 (144 mg, 459 mol) and stirred for room temperature for 30 minutes. Upon completion of the reaction, the reaction mixture was concentrated under reduced pressure to afford crude product, which was purified by preparative HPLC [Column: Acquity UPLC BEH C18, 2.1×50 mm, 1.7 micron, Mobile phase A: 0.1% formic acid in water, Mobile phase B: acetonitrile, Wavelength: 215 nm]and the fractions containing the compound were combined and lyophilized to afford the desired product 3-(3-fluorosulfonyloxy-5-methyl-phenoxy)-2,6-dioxo-piperidine (A-20) (27.5 mg, 22% yield) as an off-white solid. LCMS (ES*): m z 316.21 [M-H]-. H-NMR (400 MHz, DMSO-d6): δ 10.97 (s, 1H), 7.10 (brs, 1H), 7.01 (d, 2H), 5.31 (m, 1H), 2.76-2.64 (m, 1H), 2.63-2.59 (m, 1H), 2.34 (s, 3H), 2.25-2.19 (m, 2H).

Synthesis A21: Synthesis of 3-((2,6-dioxopiperidin-3-yl)oxy)-5-methoxyphenyl

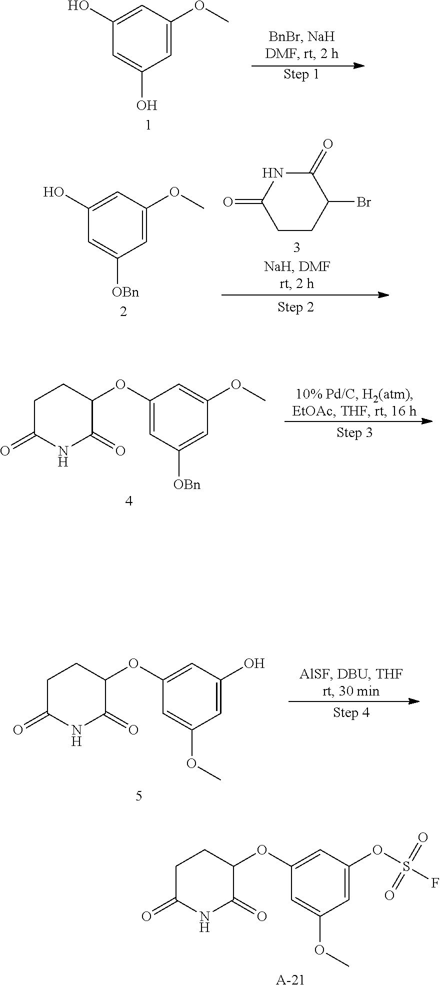

To a stirred solution of 5-methylbenzene-1,3-diol 1 (1 g, 8.1 mmol) in 15 dimethylformamide (5 mL) was added NaH (60% dispersion in oil) (640 mg, 7.8 mmol) at 0° C. and stirred at room temperature for 2 hours. Upon the completion of reaction, the reaction mixture was diluted with chilled water (10 mL) and was extracted with ethyl acetate (2×25 mL). The organic layer was dried over anhydrous sodium sulfate and concentrated under reduced pressure to afford crude product which was then purified by flash column chromatography (60-120 mess silica gel; using 15% ethyl acetate and petroleum ether as the eluent) to afford desired product 3-benzyloxy-5-methyl-phenol 2 (420 mg, 19.47% yield) as a thick oil. LCMS (ES*): m z 214.78 [M+H]+.

Step-2: To a stirred solution of 3-benzyloxy-5-methoxy-phenol 2 (900 mg, 3.9 mmol) in tetrahydrofuran (10 mL) was added NaH (60% dispersion in oil) (640 mg, 7.8 mmol), 3-bromopiperidine-2,6-dione 3 (901 mg, 4.7 mmol) consecutively and stirred at room 5 temperature for 2 hours. Upon completion of reaction, the reaction mixture was quenched with ice chilled water (10 mL) and extracted with ethyl acetate (2×25 mL). The organic layer was dried over anhydrous sodium sulfate and concentrated under reduced pressure to obtain crude product, which was then purified by flash column chromatography (60-120 mess silica gel; using 55% Ethyl acetate and petroleum ether as an eluent) to afford desired product 3-(3-benzyloxy-5-methoxy-phenoxy)piperidine-2,6-dione 4 (500 mg, 16% yield). LCMS (ES*): m z 342.16 [M+H]+.

Step-3: A stirred solution of 3-(3-benzyloxy-5-methoxy-phenoxy) piperidine-2,6-dione 4 (500 mg, 1.46 mmol) in tetrahydrofuran (5 mL), ethyl acetate (5 mL) was purged nitrogen for 5 minutes, after which was added 10% palladium on carbon (179 mg, 1.5 mmol) and stirred under H2 (30 psi) for 3 hours at room temperature. Upon completion of the reaction, the reaction mixture was filtered through celite bed and washed with ethyl acetate (2×25 mL). The combined organic layer was concentrated under reduced pressure to afford 3-(3-hydroxy-5-methoxy-phenoxy) piperidine-2,6-dione 5 (320 mg, 31% yield) as pale-yellow oil. LCMS (ES*): m z 252.08 [M+H]+.

Step-4: To a stirred solution of 3-(3-hydroxy-5-methoxy-phenoxy)piperidine-2,6-dione 5 (180 mg, 716 mol) in tetrahydrofuran (1.9 mL) was added DBU (107.14 L, 717 mol) and 4-(acetylamino)phenyl]imidodisulfuryl difluoride (109.1 mg, 716.5 mol) consecutively and stirred for room temperature for 30 minutes. Upon completion of the reaction, the reaction mixture was concentrated under reduced pressure to afford crude product. The residue obtained was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-(3-fluorosulfonyloxy-5-methoxy-phenoxy)-2,6-dioxo-piperidine (A-21) (69.9 mg, 29% yield) as an off-white solid. LCMS (ES-): m z 332.14 [M-H]-. H-NMR (400 MHz, DMSO-d6): δ 10.98 (s, 1H), 6.89 (t, J=2.2 Hz, 1H), 6.82 (t, J=2.00 Hz, 1H), 6.76 (t, J=2.2 Hz, 1H), 5.34 (q, J=5.20 Hz, 1H), 3.79 (s, 3H), 2.76-2.67 (m, 1H), 2.64-2.60 (m, 1H), 2.25-2.13 (m, 2H).

Synthesis A22: Synthesis of 3-((2,6-dioxopiperidin-3-yl)oxy)-5-methoxybenzenesulfonyl fluoride (A-22)

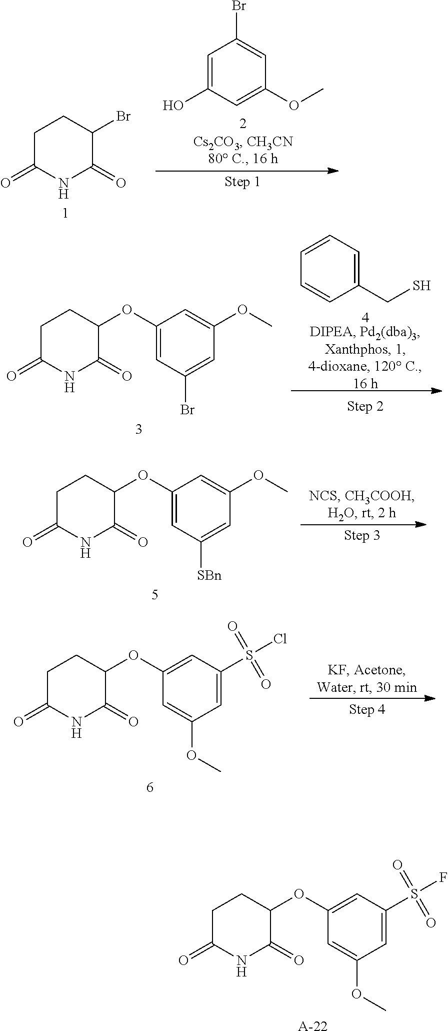

Step-1: To a stirred solution of 3-bromo-5-methoxy-phenol 2 (1.0 g, 4.9 mmol) and 3-bromopiperidine-2,6-dione 1 (1.89 g, 9.9 mmol) in acetonitrile (10 mL) added cesium 10 carbonate (4.8 g, 14.8 mmol) and stirred at 80° C. for 16 hours. Upon completion of the reaction, the reaction mixture was filtered through celite bed and was further washed with acetonitrile (30 mL). The filtrate was evaporated under reduced pressure to afford crude product which was purified by column chromatography (Davisil silica, 20% ethyl acetate in petroleum ether as an eluent) to afford 3-(3-bromo-5-methoxy-phenoxy) piperidine-2,6-dione 3 (350 mg, 21% yield) 15 as pale-yellow solid. LCMS (ES+): m z 316.05 [M+H]+.

Step-2: To a stirred solution 3-(3-bromo-5-methoxy-phenoxy) piperidine-2,6-dione 3 (300 mg, 0.95 mmol), phenylmethanethiol 4 (237 mg, 1.9 mmol) in dioxane (7 mL) were added N,N-diisopropylethylamine (370 mg, 2.9 mmol), xantphos (111 mg, 0.19 mmol) and purged with nitrogen for 5 minutes before adding Pd2(dba)3 (87.5 mg, 0.10 mmol) and stirred at 120° C. for 16 hours. Upon completion of the reaction, the reaction mixture was quenched with water (30 ml) and extracted with ethyl acetate (2×30 mL). The combined organic layer was dried over anhydrous sodium sulfate, filtered through cotton plug, and evaporated under reduced pressure to afford crude product, which was purified by column chromatography (Davisil silica, 20% ethyl acetate in petroleum ether as an eluent) to afford 3-(3-benzylsulfanyl-5-methoxy-phenoxy) piperidine-2,6-dione 5 (300 mg, 87% yield) as an off-white solid. LCMS (ES*): m z 357.88 [M+H]+.

Step-3: To a stirred solution of 3-(3-benzylsulfanyl-5-methoxy-phenoxy) piperidine-2,6-dione 5 (300 mg, 0.83 mmol) in water (3 mL) and acetic acid (12 mL) was added N-chlorosuccinimide (336 mg, 2.5 mmol) at 0° C. and stirred at room temperature for 2 hours.

Upon completion of the reaction, the reaction mixture was quenched with water (30 mL) and extracted with ethyl acetate (2×30 mL). The combined organic layer was dried over anhydrous sodium sulfate, filtered over cotton plug and evaporated under reduced pressure to afford crude 3-[(2,6-dioxo-3-piperidyl) oxy]-5-methoxy-benzenesulfonyl chloride 6 (300 mg, 60% yield) as color less oil. LCMS (ES-): m z 332.02 [M-H]-.

Step-4: To a stirred solution of 3-[(2,6-dioxo-3-piperidyl) oxy]-5-methoxy-benzenesulfonyl chloride 6 (300 mg, 0.89 mmol) in acetone (10 mL), water (10 mL) added potassium fluoride (105 mg, 1.8 mmol) and stirred at room temperature for 30 minutes. Upon completion of the reaction, the reaction mixture was diluted with water (20 mL) and extracted with ethyl acetate (2×20 mL). The organic layer was dried over anhydrous sodium sulfate, filtered through a cotton plug and evaporated under reduced pressure to obtain crude product. The crude product was purified preparative HPLC [Column: X-BRIDGE C18, 19×250 mm, 5 micron, Mobile phase A: 0.1% trifluoracetic acid in water, Mobile phase B: acetonitrile, Wavelength: 215 nm]and the fractions containing the compound were combined and lyophilized to afford the desired product 3-[(2,6-dioxo-3-piperidyl) oxy]-5-methoxy-benzenesulfonyl fluoride (A-22) (70 mg, 24% yield) as an off-white solid. LCMS (ES*): m z 316.14 [M+H]+. H-NMR (400 MHz, DMSO-d6): δ 11.00 (s, 1H), 7.33 (s, 1H), 7.21 (s, 1H), 7.15 (d, J=2.0 Hz, 1H), 5.52-5.48 (m, 1H), 3.87 (s, 3H), 2.76-2.60 (m, 2H), 2.27-2.25 (m, 2H).

Synthesis A23: Synthesis of 3-(2,6-dioxopiperidin-3-yl)phenyl sulfurofluoridate (A-23)

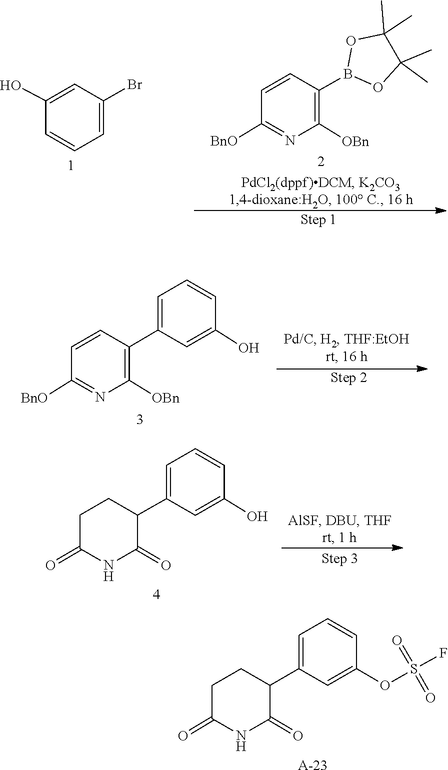

Step-1: To a stirred solution of 3-bromophenol 1 (1 g, 5.8 mmol) and 2,6-dibenzyloxy-3-(4, 4, 5, 5-tetramethyl-1, 3, 2-dioxaborolan-2-yl) pyridine 2 (4.8 g, 11.6 mmol) in a mixture of water (3 mL), dioxane (15 mL) was added potassium carbonate (2.4 g, 17.3 mmol) and degassed with argon gas for 10 minutes before adding Pd(dppf)C12 DCM (4.7 g, 5.8 mmol) and stirred at 100° C. for 16 hours. Upon completion of the reaction, the reaction mixture was filtered through 10 celite bed, washed with ethyl acetate (20 mL). The filtrate was concentrated under reduced pressure to afford crude product, which was purified by column chromatography (60-120 mesh silica gel, using 10% ethyl acetate in petroleum ether as an eluent) to afford 3-(2,6-dibenzyloxy-3-pyridyl)phenol 3 (450 mg, 16% yield) as a yellow oil. LCMS (ES*): m z 384.20 [M+H]+.

Step-2: To stirred a solution of 3-(2,6-dibenzyloxy-3-pyridyl)phenol 3 (450 mg, 1.2 mmol) in the mixture of solvent tetrahydrofuran (5 mL), ethanol (5 mL) was added 10% Palladium on activated charcoal (0.450 g, 4.2 mmol) and stirred under a hydrogen atmosphere (80 psi) at room temperatures for 16 hours. Upon completion of the reaction, the reaction mixture was filtered through celite bed, washed with ethyl acetate (2×25 mL). The filtrate was concentrated 20 under reduced pressure to obtain crude product which was purified by column chromatography (100-200 mesh silica gel, using 100% ethyl acetate as eluent) to afford 3-(3-hydroxyphenyl)piperidine-2,6-dione 4 (140 mg, 18% yield) as off- white solid. LCMS (ES+): m/z 206.09 [M+H]+.

Step-3: To a stirred solution of 3-(3-hydroxyphenyl)piperidine-2,6-dione 4 (0.130 g, 633.50 mol) in tetrahydrofuran (4 mL) was added DBU (237 pL, 1.6 mmol), (4-acetamidophenyl)(fluorosulfonyl)sulfamoyl fluoride/AISF (239 mg, 760 mol) consecutively 5 and stirred at room temperature for 1 hour. Upon completion of the reaction, the reaction mixture was concentrated under reduced pressure to obtain crude product. The crude product was purified by preparative HPLC [Column: GEMINI NX-C18, 21×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 3-(3-fluorosulfonyloxyphenyl)-2,6-dioxo-piperidine(A-23) (41 mg, 22% yield) as an off-white solid. LCMS (ES-): m/z 286.05 [M+H]+. H-NMR (400 MHz), DMSO-d6) 6: 10.91 (s, 1H), 7.50-7.59 (m, 3H), 7.42 (d, J=7.6 Hz, 1H), 4.02 (dd, J=12.4, 5.2 Hz, 1H), 2.65-2.74 (m, 1H), 2.55-2.56 (m, 1H), 2.24-2.31 (m, 1H), 2.01-2.07 (m, 1H). 15 Synthesis A24: Synthesis of 3-(((2,6-dioxopiperidin-3-yl)methyl)amino)benzenesulfonyl fluoride (A-24)

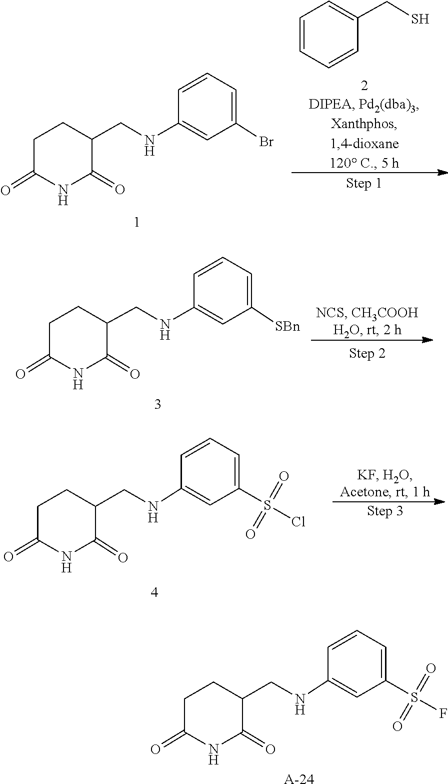

Step-1: To a degassed solution of 3-[(3-bromoanilino)methyl]piperidine-2,6-dione 1 (0.5 g, 1.7 mmol), phenylmethanethiol 2 (418 mg, 3.4 mmol) in dioxane (7.8 mL) was added N,N-diisopropylethylamine (879 pL, 5.1 mmol), xantphos (195 mg, 337 mol) and purged with nitrogen for 5 minutes before adding tris(dibenzylideneacetone)dipalladium(0) (154 mg, 168.3 mol) and stirred at 120° C. for 5 hours. Upon completion of the reaction, the reaction mixture was diluted with water (10 mL) and extracted with ethyl acetate (2×25 mL). The combined organic layer was dried over sodium sulfate and evaporated under reduced pressure to obtain crude product, which was purified over column chromatography (Davisil silica; 20% ethyl acetate in petroleum ether as eluent) to afford 3-[(3-benzylsulfanylanilino)methyl]piperidine-2,6-dione 3 (0.53 g, 86% yield). LCMS (ES*): m z 341.166 [M+H]+.

Step-2: To a stirred solution of 3-[(3-benzylsulfanylanilino)methyl]piperidine-2,6-dione 3 (0.28 g, 810.7 mol) in acetic acid (5 mL), water (1 mL) was added N-chlorosuccinimide (217 mg, 1.6 mmol) at 0° C. and reaction mixture was stirred at room temperature for 2 hours. Upon 10 completion of the reaction, the reaction mixture was diluted with water (25 mL) and extracted with ethyl acetate (2×25 mL). The combined organic layer was dried over anhydrous sodium sulfate and concentrated under reduced pressure to obtain crude product 3-[(2,6-dioxo-3-piperidyl) methylamine]benzenesulfonyl chloride 4 (0.4 g, 31% yield) as pale-yellow oil.

LCMS (ES-): m z 314.96 [M-H]-. 15 Step-3: To a stirred solution of 3-[(2,6-dioxo-3-piperidyl)methylamino]benzenesulfonyl chloride 4 (0.2 g, 631.39 mol) in acetone (8 mL), water (8 mL) was added potassium fluoride (74 mg, 1.3 mmol) and stirred at room temperature for 1 hour. Upon completion of the reaction, the reaction mixture was concentrated under reduced pressure to obtain crude product which was purified preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-[(2,6-dioxo-3-piperidyl)methylamino]benzenesulfonyl fluoride (A-24) (26 mg, 13% yield) as an off-white solid. LCMS (ES+): m z 301.07 [M+H]+.H-NMR (400 MHz), DMSO-d6) 6: 10.76 (s, 1H), 7.45 (t, J=8.00 Hz, 1H), 7.22-7.17 (m, 2H), 7.09 (d, J=2, 12.40 Hz, 1H), 6.62 (t, J=11.6, 12.40 Hz, 1H), 3.57-3.51 (m, 1H), 3.37-3.27 (m, 1H), 2.81-2.74 (m, 1H), 2.62-2.50 (m, 2H), 2.03-1.96 (m, 1H), 1.85-1.74 (m, 1H).

Synthesis A25: Synthesis of 4-(2,6-dioxopiperidin-3-yl)phenyl sulfurofluoridate (A-25)

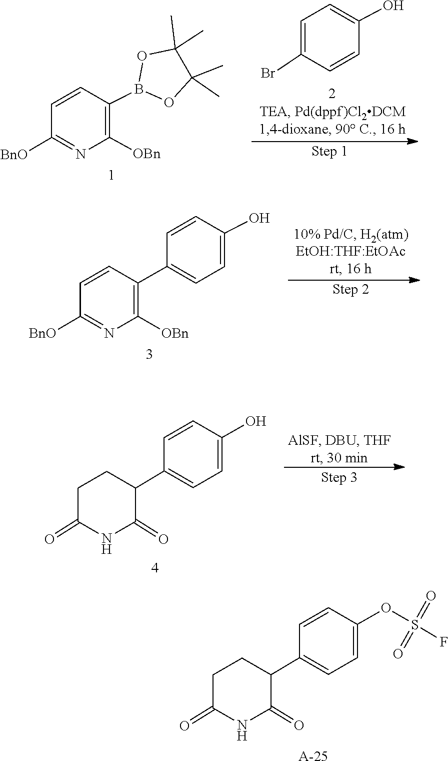

Step-1: To a stirred solution of 2,6-dibenzyloxy-3-(4,4,5,5-tetramethyl-1,3,2-dioxaborolan-2-yl) pyridine 1 (2 g, 4.8 mmol), 4-bromophenol 2 (1.66 g, 9.6 mmol) in 1,4-dioxane (10 mL) and water (2 mL) was added triethylamine (2.00 mL,14.4 mmol,) and purged with N2 gas for 10 minutes. After which was added Pd(dppf)C12 DCM (196 mg, 239.6 mol) and stirred at 90° C. for 16 hours. After completion of the reaction, the reaction mixture was evaporated and purified by column chromatography (100-200 mesh silica gel; using 40% ethyl acetate in hexane as eluent) to afford 4-(2,6-dibenzyloxy-3-pyridyl) phenol 3 (1.20 g, 57.46% yield) as an off-white solid. LCMS (ES+): m z 384.91 [M+H]*.

Step-2: To a stirred solution of 4-(2,6-dibenzyloxy-3-pyridyl) phenol 3 (200 mg, 522 mol) in 15 tetrahydrofuran (2 mL), ethanol (2 mL) and ethyl acetate (2 mL) was added 20% Pd/C (222.03 mg, 2.09 mmol) and stirred under H2 (180 psi) at room temperature for16 hours. Upon completion of the reaction, the reaction mixture was filtered over a celite bed and washed with ethyl acetate (2×25 mL). The filtrate was evaporated under reduced pressure to afford the crude product. The crude product was purified by column chromatography (100-200 mesh silica gel using 50% Ethyl acetate in petroleum ether as eluent) to afford 3-(4-hydroxyphenyl) piperidine-2,6-dione 4 (70 mg, 57% yield) as an off-white solid. LCMS (ES+): m z 205.70 [M+H]+.

Step-3: To a stirred solution of 3-(4-hydroxyphenyl) piperidine-2,6-dione 4 (70 mg, 341 mol) in tetrahydrofuran (1.0 mL) was added (4-acetamidophenyl)(fluorosulfonyl)sulfamoyl fluoride (129 mg, 409 mol) and 2,3,4,6,7,8,9,10-octahydropyrimido[1,2-a]azepine (127.53 L, 853 mol) and stirred at room temperature for 30 minutes. After completion of the reaction, the reaction mixture was concentrated under reduced pressure to afford the crude product. The crude product was purified by preparative HPLC [Column: X SELECT C18, 19×250 mm, 5 micron, Mobile phase A: 0.1% Formic acid in water, Mobile phase B: Acetonitrile, Wavelength: 215 nm]and the fractions containing the compound were combined and 10 lyophilized to afford the desired product 3-(4-fluorosulfonyloxyphenyl)-2,6-dioxo-piperidine (A-25) (24 mg, 24% yield) as white solid. LCMS (ES-): m z 286.05 [M-H]-. H-NMR (400 MHz), DMSO-d6) δ: 10.90 (s, 1H), 7.57 (d, J=8.40 Hz, 2H), 7.46 (d, J=8.80 Hz, 2H), 4.02-3.98 (m, 1H), 2.65-2.67 (m, 1H), 2.55-2.56 (m, 1H), 2.21-2.22 (m, 1H), 2.02-2.03 (m, 1H). 15 Synthesis A26: Synthesis of 2-(1-methyl-2,6-dioxopiperidin-3-yl)-1,3-dioxoisoindolin-5-yl sulfurofluoridate (A-26)

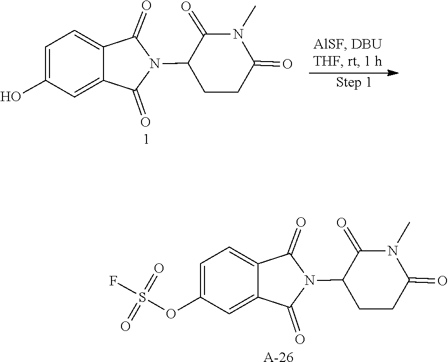

Step-1: To a stirred solution of 5-hydroxy-2-(1-methyl-2,6-dioxo-3-piperidyl)isoindoline-1,3-dione 1 (0.2 g, 694 mol) in tetrahydrofuran (6 mL) was added (4-acetamidophenyl)(fluorosulfonyl)sulfamoyl fluoride (262 mg, 833 mol) and 2,3,4,6,7,8,9,10-octahydropyrimido[1,2-a]azepine (259.40 pL, 1.73 mmol) consecutively and stirred at room temperature for 1 hour. After completion of the reaction, the reaction mixture was diluted with water (10 mL) and extracted with ethyl acetate (2×20 mL). The combined organic layer was dried over sodium sulfate and concentrated under reduced pressure to afford the crude product, which was purified by preparative HPLC [Column: KINETICS EVO C18, 21×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 5-fluorosulfonyloxy-2-(1-methyl-2,6-dioxo-3-piperidyl)-1,3-dioxo-isoindoline (A-26) (52.5 mg, 20% yield) as an off-white solid. LCMS (ES+): m/z 371.26 [M+H]+. ¹H NMR (400 MHz), DMSO-d6) δ: 8.31 (d, J=1.6 Hz, 1H), 8.18-8.13 (m, 2H), 5.29-5.24 (m, 1H), 3.03 (s, 3H), 2.99-2.92 (m, 1H), 2.82-2.76 (m, 1H), 2.60-2.48 (m, 1H), 2.10-2.05 (m, 1H).

Synthesis A27: Synthesis of 5-((2,6-dioxopiperidin-3-yl)oxy)-2-methoxyphenyl sulfurofluoridate (A-27)

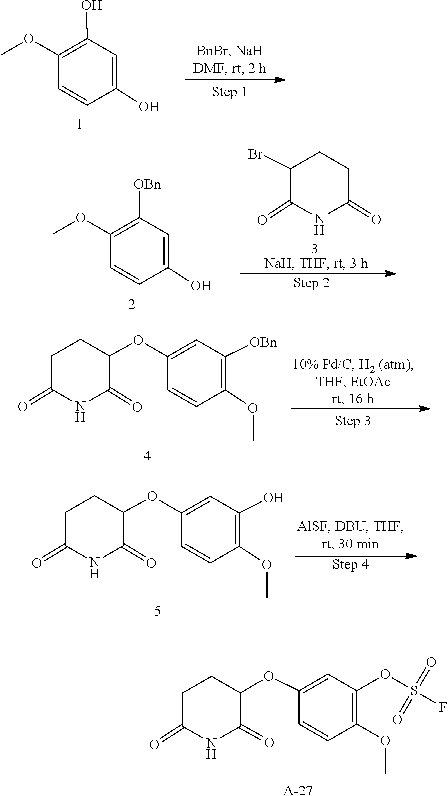

10 Step-1: To a solution of 4-methoxybenzene-1,3-diol 1 (2 g, 14.3 mmol) in dimethylformamide (20 mL) was added NaH (60% dispersion in mineral oil) (685 mg, 28.5 mmol), benzyl bromide (1.70 mL, 14.3 mmol) consecutively and stirred at room temperature for 2 hours. Upon completion of reaction, the reaction mixture was quenched with ice cold water (20 mL) and 15 extracted with ethyl acetate (2×50 ml). The combined organic layer was dried over anhydrous sodium sulfate and concentrated under reduced pressure to afford the crude material, which was purified by flash column chromatography (Davisil silica, using ~30% ethyl acetate in petroleum ether as eluent) to afford 3-benzyloxy-4-methoxy-phenol 2 (2.9 g, 84% yield) as an off-white solid. LCMS (ES-): m/z 229.11 [M-H]-. 20 Step-2: To a stirred solution of 3-benzyloxy-4-methoxy-phenol 2 (537 mg, 2.3 mmol) in tetrahydrofuran (10 mL) was added NaH (60% dispersion in mineral oil) (112 mg, 4.7 mmol), 3-bromopiperidine-2,6-dione 3 (537 mg, 2.80 mmol) at 0° C. and stirred at room temperature for 3 hours. After the completion of the reaction, the reaction mixture was quenched with ice chilled water (10 mL) and extracted with ethyl acetate (2×30 mL). The combined organic layer was dried over anhydrous sodium sulfate and concentrated under reduced pressure to afford crude product, which was purified by column chromatography (100-200 mesh silica gel using 40% ethyl acetate in petroleum ether as eluent) 3-(3-benzyloxy-4-methoxy-phenoxy)piperidine-2,6-dione 4 (0.3 g, 24% yield) as an off-white solid. H-NMR (400 MHz), DMSO-d6) δ: 10.90 (s, 1H), 7.45-7.33 (m, 5H), 6.87 (d, J=9.2 Hz, 1H), 6.77 (d, J=2.8 Hz, 1H), 6.57-6.54 (m, 1H), 5.05 (s, 3H), 3.71 (s, 3H), 2.68-2.59 (m, 2H), 2.14-2.02 (m, 2H).

Step-3: A stirred solution of 3-(3-benzyloxy-4-methoxy-phenoxy)piperidine-2,6-dione 4 (0.2 10 g, 586 mol) in ethyl acetate (1.5 mL), tetrahydrofuran (1.5 mL) was added 10% Palladium on carbon (62 mg, 586 mol) and stirred under H2 (1 atm) at room temperature for 16 hours. After completion of reaction, the reaction mixture was filtered through celite bed and washed with ethyl acetate (20 mL). The filtrate was concentrated under reduced pressure to afford 3-(3-hydroxy-4-methoxy-phenoxy) piperidine-2,6-dione 5 (0.15 g, 60% yield) as light brown oil. 15 LCMS (ES-): m z 250.02 [M-H]-.

Step-4: To a stirred solution of 3-(3-hydroxy-4-methoxy-phenoxy)piperidine-2,6-dione 5 (0.15 g, 597 mol) in tetrahydrofuran (4 mL) were added (4-acetamidophenyl)(fluorosulfonyl)sulfamoyl fluoride (225 mg, 716 mol) and 2,3,4,6,7,8,9,10-octahydropyrimido[1,2-a]azepine (223.21 L, 1.49 mmol) and stirred at room temperature for 30 minutes. After completion of the reaction, the reaction mixture was diluted with saturated sodium bicarbonate solution (10 mL) and extracted with ethyl acetate (2×20 mL). The organic layer was dried over sodium sulfate and concentrated under reduced pressure to afford the crude product, which was purified by preparative HPLC [Column: GEMINI NX C8, 19×250 mm, 5 micron, Mobile phase A: 0.1% formic acid in water, Mobile phase B: Acetonitrile, Wavelength: 215 nm]and the fractions containing the compound were combined and lyophilized to afford the desired product 3-(3-fluorosulfonyloxy-4-methoxy-phenoxy)-2,6-dioxo-piperidine (A-27) (52.5 mg, 26% yield) as an off-white solid. LCMS (ES-): m z 332.09 [M-H]-. ¹H-NMR (400 MHz, DMSO-d6): δ 10.95 (s, 1H), 7.34 (d, J=2.8 Hz, 1H), 7.27 (d, J=9.2 Hz, 1H), 7.15-7.12 (m, 1H), 5.21-5.17 (m, 1H), 3.86 (s, 3H), 2.74-2.58 (m, 2H), 2.25-2.11 (m, 2H).

Synthesis A28: Synthesis of 6-((2,6-dioxopiperidin-3-yl)oxy)pyridin-3-yl sulfurofluoridate (A-28)

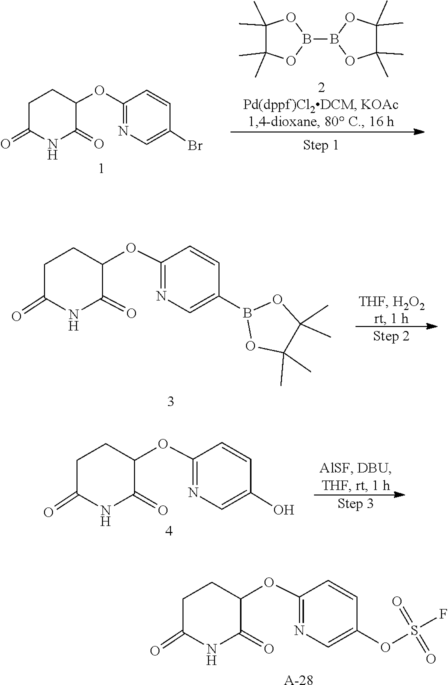

Step-1: A stirred solution of 3-[(5-bromo-2-pyridyl)oxy]piperidine-2,6-dione 1 (0.5 g, 1.8 mmol), potassium acetate (328.90 pL, 5.26 mmol), 4,4,5,5-tetramethyl-2-(4,4,5,5-tetramethyl-1,3,2-dioxaborolan-2-yl)-1,3,2-dioxaborolane 2 (786 mg, 3.1 mmol) in 1,4-dioxane (5 mL) was purged with N2 for 15 minutes. 1,1′-bis(diphenylphosphino)ferrocene]dichloropalladium(II) 10 (72 mg, 87.7 mol) was added to the reaction and the reaction was stirred at 80° C. for 16 hours.

After the completion of reaction, the reaction mixture was diluted with water (10 mL) and was extracted with ethyl acetate (2×25 mL). The combined organic layer was dried over anhydrous sodium sulfate and concentrated under reduced pressure to afford the crude product, which was purified by flash column chromatography (60-120 silica gel; using 30% ethyl acetate in 15 petroleum ether as an eluent) to afford 3-[[5-(4,4,5,5-tetramethyl-1,3,2-dioxaborolan-2-yl)-2-pyridyl]oxy]piperidine-2,6-dione 3 (300 mg, 25% yield). LCMS (ES+): m z 333.87 [M+H]+.

Step-2: To a stirred solution of 3-[[5-(4,4,5,5-tetramethyl-1,3,2-dioxaborolan-2-yl)-2-pyridyl]oxy]piperidine-2,6-dione 3 (80 mg, 241 mol) in tetrahydrofuran (5.00 mL) added hydrogen peroxide 35% (22.14 L, 722.55 mol,) at 0° C. and stirred at room temperature for 1 hour. After completion of the reaction, the reaction mixture was diluted with water (20 mL) and extracted with ethyl acetate (3×10 mL). The combined organic layer was dried over anhydrous sodium sulfate and concentrated under reduced pressure to afford 3-[(5-hydroxy-2-pyridyl)oxy]piperidine-2,6-dione 4 (55 mg, 67% yield). LCMS (ES+): m/z 223.81 [M+H]+. Step-3: To a stirred solution of 3-[(5-hydroxy-2-pyridyl)oxy]piperidine-2,6-dione 4 (80 mg, 360 mol) in tetrahydrofuran (2 mL) was added (4-acetamidophenyl)(fluorosulfonyl)sulfamoyl fluoride (136 mg, 432 mol) and 2,3,4,6,7,8,9,10-octahydropyrimido[1,2-a]azepine (134.61 L, 900 mol) and stirred at room temperature for 1 hour. After completion of the reaction, the reaction mixture was diluted with water (10 mL) and extracted with ethyl acetate (2×30 mL). The organic layer was dried over sodium sulfate and concentrated under reduced pressure to afford the crude product, which was purified by preparative HPLC [Column: GEMINI NX C8, 19×250 mm, 5 micron, Mobile phase A: 0.1% 10 formic acid in water, Mobile phase B: Acetonitrile, Wavelength: 215 nm]and the fractions containing the compound were combined and lyophilized to afford the desired product 2-[(2,6-dioxo-3-piperidyl)oxy]-5-fluorosulfonyloxy-pyridine (A-28) (11 mg, 10% yield) as an off-white solid. LCMS (ES*): m z 305.2 [M+H]+. H-NMR (400 MHz), DMSO-d6) δ: 10.96 (s, 1H), 8.48 (d, J=2.8 Hz, 1H), 8.12-8.09 (m, 1H), 7.12 (d, J=9.2 Hz, 1H), 5.96-5.91 (m, 1H), 2.89-2.79 (m, 1H), 2.63-2.57 (m, 1H), 2.26-2.19 (m, 2H)).

Synthesis A29: Synthesis of 3-(2,6-dioxopiperidin-3-yl)-2-oxo-2,3-dihydrobenzo[d]oxazole-7-sulfonyl fluoride (A-29)

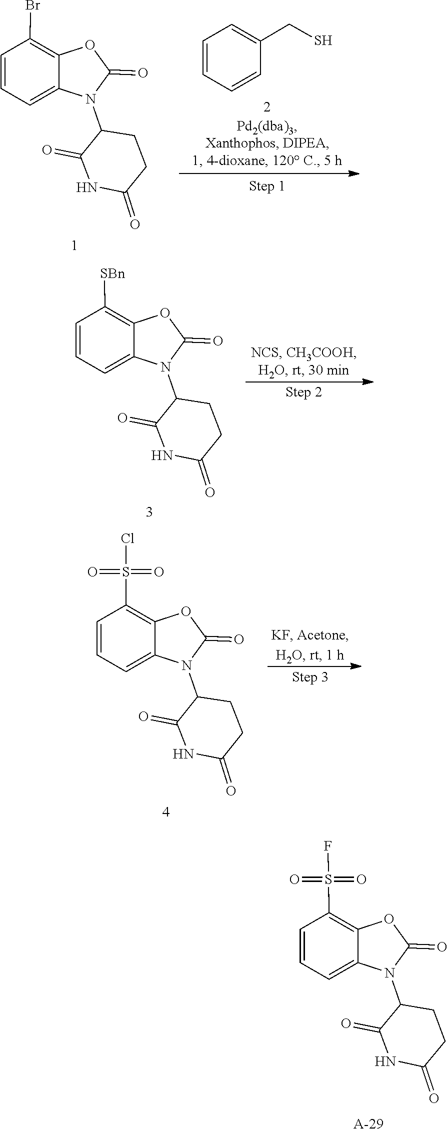

Step-1: To a stirred solution of 3-(7-bromo-2-oxo-1,3-benzoxazol-3-yl)piperidine-2,6-dione 1 (0.5 g, 1.5 mmol), phenylmethanethiol 2 (382 mg, 3.1 mmol) in 1,4-dioxane (8 mL) was added N,N-diisopropylethylamine (803.63 L, 4.61 mmol), xantphos (178 mg, 308 mol) and 5 degassed with argon gas for 10 minutes before adding Pd2(dba)3 (141 mg, 154 mol) and stirred at 120° C. for 5 hours. After completion of reaction, the reaction mixture was filtered through celite bed and washed with ethyl acetate (20 mL). The filtrate was concentrated under reduced pressure to afford the crude product, which was purified by flash column chromatography (Davisil silica; using 60% ethyl acetate in petroleum ether as eluent) to afford 3-(7-benzylsulfanyl-2-oxo-1,3-benzoxazol-3-yl)piperidine-2,6-dione 3 (0.35 g, 61% yield) as a pale-yellow solid. LCMS (ES): m z 369.11 [M+H]*.

Step-2: To a stirred solution of 3-(7-benzylsulfanyl-2-oxo-1,3-benzoxazol-3-yl)piperidine-2,6-dione 3 (0.2 g, 543 mol) in water (2 mL), acetic acid (10 mL) was added N-chlorosuccinimide 15 (145 mg, 1.1 mmol) at 0° C. and stirred at room temperature for 1 hour. After completion of the reaction, the reaction mixture was diluted with saturated sodium bicarbonate solution (10 mL) and extracted with ethyl acetate (2×25 mL). The combined organic layer was dried over sodium sulfate and concentrated under reduced pressure to afford the crude product 3-(2,6-dioxo-3-piperidyl)-2-oxo-1,3-benzoxazole-7-sulfonyl chloride 4 (0.3 g, 54% yield) as a pale-yellow solid. LCMS (ES-): m z 342.97 [M-H]-.

Step-3: To a stirred solution of 3-(2,6-dioxo-3-piperidyl)-2-oxo-1,3-benzoxazole-7-sulfonyl chloride 4 (0.3 g, 870 mol) in acetone (5 mL), water (5 mL) was added potassium fluoride (101 mg, 1.7 mmol) at room temperature and stirred for 1 hour. After completion of the reaction, the reaction mixture was concentrated under reduced pressure to afford crude product, which was purified was purified by preparative HPLC [Column: X-Bridge C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-(2,6-dioxo-3-piperidyl)-2-oxo-1,3-benzoxazole-7-sulfonyl fluoride (A-29) (25.6 mg, 9% yield) as an off-white solid. LCMS (ES-): m z 327.11 [M-H]-. H-NMR (400 MHz), DMSO-d6) δ: 11.29 (s, 1H), 7.85 (d, J=8.0 Hz, 1H), 7.78 (d, J=8.0 Hz, 1H), 7.56 (t, J=8.0 Hz, 1H), 5.54-5.49 (m, 1H), 2.93-2.84 (m, 1H), 2.73-2.62 (m, 2H), 2.24-2.07 (m, 1H).

Synthesis A30: Synthesis of 3-(2,4-dioxotetrahydropyrimidin-1(2H)-yl)-1-methyl-1H-indazol-7-yl sulfurofluoridate (A-30)

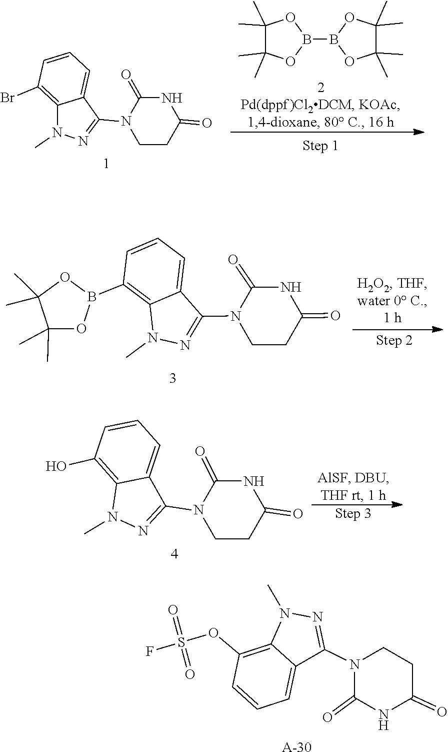

5 Step-1: A stirred solution of 1-(7-bromo-1-methyl-indazol-3-yl)hexahydropyrimidine-2,4-dione 1 (500 mg, 1.6 mmol), potassium acetate (193.45 L, 3.1 mmol) and 4,4,5,5-tetramethyl-2-(4,4,5,5-tetramethyl-1,3,2-dioxaborolan-2-yl)-1,3,2-dioxaborolane 2 (786 mg, 3.1 mmol) in 1,4-dioxane (4.9 mL) was purged with N2 for 10 minutes before addition of [1,1′-bis(diphenylphosphino)ferrocene]dichloropalladium(II) complex (126 mg, 155 mol) and stirred at 90° C. for 16 hours. After completion of the reaction, the reaction mixture was filtered through celite and washed with ethyl acetate (25 mL). The filtrate was concentrated under reduced pressure to afford crude product, which was purified by flash column chromatography (Davisil silica; using 55% ethyl acetate in petroleum ether as eluent) to afford 1-[1-methyl-7-(4,4,5,5-tetramethyl-1,3,2-dioxaborolan-2-yl) indazol-3-yl]hexahydropyrimidine-2,4-dione 3 (200 mg, 18% yield). LCMS (ES+): m z 371.22[M+H]+.

Step-2: To a stirred solution of 1-[1-methyl-7-(4,4,5,5-tetramethyl-1,3,2-dioxaborolan-2-yl) indazol-3-yl]hexahydropyrimidine-2,4-dione 3 (200 mg, 540 mol) in tetrahydrofuran (30 mL) was added 35% hydrogen peroxide (1.20 mL, 39.2 mmol) and the reaction was stirred at 0° C. for 1 hour. Upon completion of the reaction, the reaction mixture was diluted with 10% sodium bisulfite solution (10 mL) and extracted with diethyl ether (2×25 mL). The organic layer was concentrated under reduced pressure to afford crude product 4 which was directly taken to the next step without further purification LCMS (ES+): m z 261.11 [M+H]*. Step-3: To a stirred solution of 1-(7-hydroxy-1-methyl-indazol-3-yl) hexahydropyrimidine-2,4-dione 4 (100 mg, 384 mol) in tetrahydrofuran (1.9 mL) added DBU (144 L, 960.6 mol,) and (4-acetamidophenyl) (fluorosulfonyl)sulfamoyl fluoride (145 mg, 461 mol) and stirred at room temperature for 1 hour. Upon completion of the reaction, the reaction mixture was diluted with water (10 mL) and extracted with ethyl acetate (2×25 mL). The organic layer was dried over anhydrous sodium sulfate and evaporated under reduced pressure to afford the crude product. The crude was then was purified by preparative HPLC [Column: KINETIX C-18, 21 x 250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 3-(2,4-dioxohexahydropyrimidin-1-yl)-7-fluorosulfonyloxy-1-methyl-indazole (A-30) (4.6 mg, 3% yield) as an off-white solid. LCMS (ES*): m/z 343.24 [M+H]+. H-NMR (400 MHz), DMSO-d6) δ: 10.66 (s, 1H), 7.81 (d, J=8.0 Hz, 1H), 7.70 (d, J=6.8 Hz, 1H), 7.24 (t, J=8.0 Hz, 1H), 4.15 (s, 3H), 3.95 (t, J=6.8 Hz, 2H), 2.77 (t, J=6.8 Hz, 2H).

Synthesis A31: Synthesis of 5-((2,6-dioxopiperidin-3-yl)amino)-2-(piperidin-4-yl)phenyl sulfurofluoridate (A-31)

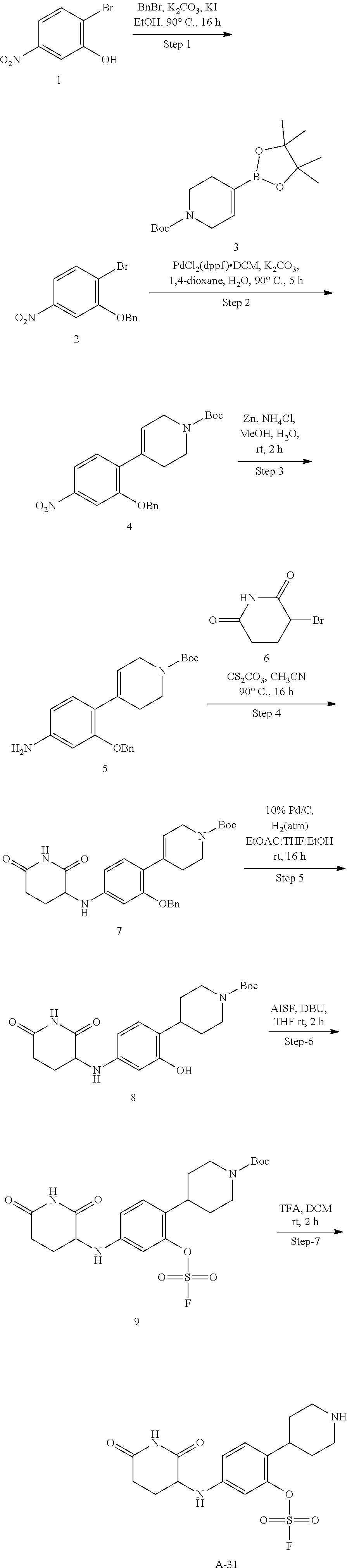

Step-1:

To the stirred mixture of 2-bromo-5-nitro-phenol 1 (5.0 g, 23.0 mmol) in ethanol (67 5 mL) was added potassium carbonate (3.80 g, 27.5 mmol), benzyl bromide (3.6 mL, 29.8 mmol) and potassium iodide (611 L, 11.5 mmol,) and stirred at 90° C. for 16 hours. Upon completion, the reaction mixture was evaporated under reduced pressure to obtain crude material. The crude residue was suspended in water (100 mL) and ethyl acetate (100 mL). The organic layer was separated and dried over sodium sulfate and evaporated under reduced pressure to afford the 10 crude product. The crude product was purified by flash chromatography (60-120 mesh silica gel using 20% ethyl acetate in petroleum ether as eluent) to afford the 2-benzyloxy-1-bromo-4-nitro-benzene 2 (5.02 g, 64% yield) as off yellowish solid.¹H NMR (400 MHz), DMSO-d6) δ: 7.93-7.95 (m, 2H), 7.77-7.78 (m, 1H), 1.00 (d, J=8.80 Hz, 1H), 7.44 (t, J=7.20 Hz, 2H), 7.32-7.33 (m, 2H), 5.39 (s, 2H). 15 Step-2: To a stirred solution of 2-benzyloxy-1-bromo-4-nitro-benzene 2 (5.0 g, 16.2 mmol), tert-butyl4-(3,3,4,4-tetramethyl-1,2,5-bromadioxolan-1-yl)-3,6-dihydro-2H-pyridine-Icarboxylate 3 (7.4 g, 19.5 mmol) in a mixture of dioxane (20 mL) and water (5 mL) was added potassium carbonate (6.7 g, 48.7 mmol) and degassed with N2 for 10 minutes. After degassing, 20 [1,1′-bis(diphenylphosphino)ferrocene]dichloropalladium(II) complex (1.3 g, 1.6 mmol) was added and the reaction mixture was stirred at 90° C. for 5 hours. Upon completion of the reaction, the reaction mixture was filtered through a celite bed and washed with dichloromethane (2×20 mL). The combined organic layer was washed with saturated sodium chloride solution (20 mL) and concentrated under reduced pressure to obtain crude product.

The crude product was purified by column chromatography (Davisil Silica, using ~8% of ethyl acetate in pet-ether as an eluent) to afford tert-butyl 4-(2-benzyloxy-4-nitro-phenyl)-3,6-dihydro-2H-pyridine-1-carboxylate 4 (4.5 g, 64% yield) as an off-white solid. LCMS (ES-): m z 311.22 [M-Boc]-.

Step-3: To a stirred solution of tert-butyl 4-(2-benzyloxy-4-nitro-phenyl)-3,6-dihydro-2H-pyridine-1-carboxylate 4 (1.2 g, 2.9 mmol) in methanol (50 mL), water (20 mL) was added zinc dust (13.7 g, 209 mmol, 1.9 mL), ammonium chloride (7.31 mL, 209 mmol) and stirred at room temperature for 2 hours. Upon completion of the reaction, the reaction mixture was passed through celite bed and washed with ethyl acetate (100 mL). The filtrate was dried over 10 anhydrous sodium sulfate, concentrated under reduced pressure to afford tert-butyl 4-(4-amino-2-benzyloxy-phenyl)-3,6-dihydro-2H-pyridine-1-carboxylate 5 (0.72 g, 1.6 mmol, 56% yield) as pale-brown color semisolid LCMS (ES): m/z 381.28 [M+H]*.

Step-4: To the stirred solution of tert-butyl 4-(4-amino-2-benzyloxy-phenyl)-3,6-dihydro-2H-pyridine-1-carboxylate 5 (1.0 g, 2.6 mmol) in acetonitrile (20 mL) was added cesium carbonate (2.6 g, 7.9 mmol) and 3-bromopiperidine-2,6-dione 6 (2.5 g, 13.1 mmol) at room temperature.

The reaction mixture was heated to 90° C. for 16 hours. Upon completion of the reaction, the reaction mixture was filtered over celite bed and washed with ethyl acetate (2×25 mL). The organic layer was evaporated under reduced pressure to obtain crude product. The crude product was purified by column purification (Davisil Silica; using ~45% of Ethyl acetate in Pet-ether as an eluent) to afford tert-butyl 4-[2-benzyloxy-4-[(2,6-dioxo-3-piperidyl) amino]phenyl]-3, 6-dihydro-2H-pyridine-1-carboxylate 7 (0.76 g, 47.% yield) as an off-white solid.

LCMS (ES-): m z 490.13 [M-H]-.

Step-5: A stirred solution of tert-butyl 4-[2-benzyloxy-4-[(2,6-dioxo-3-piperidyl)amino]phenyl]-3,6-dihydro-2H-pyridine-1-carboxylate 7 (600 mg, 1.2 mmol) in a mixture of ethyl acetate (5.0 mL), ethanol (5.0 mL) and tetrahydrofuran (1.3 mL) was degassed using argon gas for 10 minutes. After, 10% Pd/C (123 mg, 1.2 mmol; 55% wetted) and stirred under hydrogen atmosphere (10 psi) at room temperature for 16 hours. Upon completion, the reaction mixture was filtered using celite bed and washed with ethyl acetate (2×20 mL). The organic layer was concentrated under reduced pressure to obtain crude product which was purified by flash column chromatography (200-430 mesh silica gel; using 60% of ethyl acetate in pet-ether as eluent) to afford tert-butyl 4-[4-[(2,6-dioxo-3-piperidyl)amino]-2-hydroxy-phenyl]piperidine-1-carboxylate 8 (170 mg, 32% yield) as brown solid. LCMS (ES-): m/z 402.21 [M-H]-.

Step-6: To a stirred solution of tert-butyl 4-[4-[(2,6-dioxo-3-piperidyl) amino]-2-hydroxy-phenyl]piperidine-1-carboxylate 8 (170 mg, 421 mol) in tetrahydrofuran (16 mL) were added (4-acetamidophenyl)(fluorosulfonyl)sulfamoyl fluoride (159 mg, 506 mol) and 2,3,4,6,7,8,9,10-octahydropyrimido[1,2-a]azepine (160.36 mg, 1.05 mmol, 157.52 L). The reaction mixture was stirred room temperature for 2 hours. Upon completion of the reaction, the reaction mixture was filtered through celite bed and washed with ethyl acetate (3×5 mL). 10 The organic layer was concentrated under reduced pressure to obtain tert-butyl 4-[4-[(2,6-dioxo-3-piperidyl)amino]-2-fluorosulfonyloxy-phenyl]piperidine-1-carboxylate 9 (180 mg, 68% yield) as colorless semi solid LCMS (ES-): m/z 484.29 [M-H]-.

Step-7: To the stirred solution tert-butyl 4-[4-[(2,6-dioxo-3-piperidyl)amino]-2-fluorosulfonyloxy-phenyl]piperidine-1-carboxylate 9 (220 mg, 453 mol) in dichloromethane (5.0 mL) added trifluoroacetic acid (34.67 L, 453.1 mol) and stirred at room temperature for 2 hours. Upon completion of the reaction, the reaction mixture was concentrated under reduced pressure to obtain crude product. The crude product was purified by preparative HPLC [Column: Sun Fire C-18, 19×150 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 3-[3-fluorosulfonyloxy-4-(4-piperidyl) anilino]-2,6-dioxo-piperidine (A-31) (51.5 mg, 23% yield) as an off-white solid. LCMS (ES*): m/z 386.32 [M+H]+. ¹H NMR (400 MHz), DMSO-d6) δ: 10.82 (s, 1H), 7.12 (d, J=8.80 Hz, 1H), 6.79 (t, J=3.60 Hz, 2H), 6.46 (d, J=8.00 Hz, 1H), 4.36-4.38 (m, 1H), 3.37-0.00 (m, 2H), 2.98-2.99 (m, 3H), 2.71-2.72 (m, 1H), 2.57-2.60 (m, 2H), 2.07-2.10 (m, 1H), 1.92-1.93 (m, 1H), 1.89-1.90 (m, 4H).

Synthesis A32: Synthesis of 5-(2,6-dioxopiperidin-3-yl)-2-(piperidin-4-yl)phenyl sulfurofluoridate (A-32) 0 0 N 20 04 Boc 2 NBoc BnO N OBn Pd(dppf)C1₂.DCM, Pd(dppf)C1₂.-DCM, Na₂CO₃, 1,4-dioxane:H₂O Na₂CO₃, 1,4-dioxane:H₂O r 90° C., 1 h 90° C., 2 h Br OH Step 1 Br 3 OH Step 2 N‘Boc N’Boc AISF,DBU, THF, nOOH Pd/C, Pt02, H₂(atm) OH rt, 1 h EtOH, rt, 3 h O BnO N OBn 0 N 0 Step 4 Step 3 H 6 NBoc NH TFA, DCM/O 0 rt, 2 h O=S=0 O N 0 O=S=0 0 N O I Step5 H F H F A-32

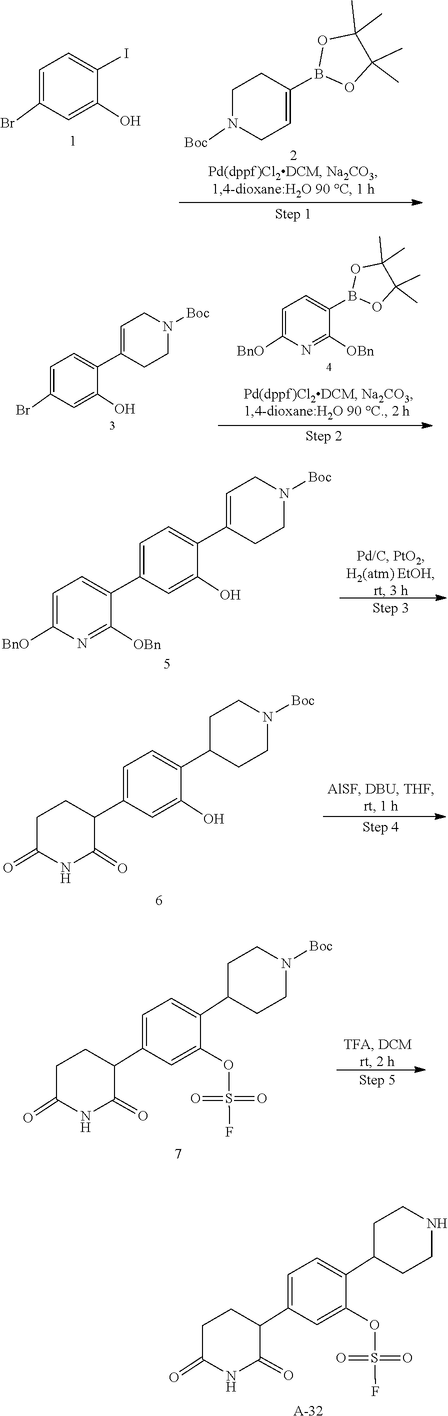

Step-1: To a stirred solution of 5-bromo-2-iodo-phenol 1 (2 g, 6.7 mmol), tert-butyl 4-(4,4,5,5-tetramethyl-1,3,2-dioxaborolan-2-yl)-3,6-dihydro-2H-pyridine-1-carboxylate 2 (2.07 g, 6.7 mmol) in dioxane (20 mL) and water (4 mL) was added sodium carbonate (1.8 g, 16.78 mmol). 10 The reaction mixture was purged with nitrogen for 10 minutes, after which [1,1′-bis(diphenylphosphino)ferrocene]dichloropalladium(II)complex with dichloromethane (546 mg, 669 mol) was added and the reaction was stirred for 1 hour at 90° C. Upon completion of the reaction, the reaction mixture was filtered through celite and washed with ethyl acetate (2×25 mL). The filtrate was concentrated under reduced pressure to obtain the crude product.

The crude product was purified by column chromatography (Davisil silica; using 16% ethyl acetate in Pet ether as an eluent) to afford tert-butyl 4-(4-bromo-2-hydroxy-phenyl)-3,6-dihydro-2H-pyridine-1-carboxylate 3 (900 mg, 35% yield) as a colorless solid. LCMS (ES-): m z 352.08 [M-H]-.

Step-2: To a stirred solution of tert-butyl 4-(4-bromo-2-hydroxy-phenyl)-3,6-dihydro-2H-pyridine-1-carboxylate 3 (800 mg, 2.3 mmol), 2,6-dibenzyloxy-3-(4,4,5,5-tetramethyl-1,3,2-dioxaborolan-2-yl)pyridine 4 (972 mg, 2.3 mmol) ) in dioxane (16 mL) and water (3.2 mL) 5 was added sodium carbonate (718 mg, 6.8 mmol) and purged with N2 for 5 minutes. After, PdCl2(dppf) DCM (184 mg, 226 mol) and stirred at 90° C. for 2 hours. Upon completion of the reaction, the reaction mixture was filtered through celite and washed with ethyl acetate (2×20 mL). The filtrate was concentrated under reduced pressure to afford the crude product, which was purified by column chromatography (Davisil silica; using 20% ethyl acetate in Pet 10 ether as an eluent) to afford tert-butyl 4-[4-(2,6-dibenzyloxy-3-pyridyl)-2-hydroxy-phenyl]-3,6-dihydro-2H-pyridine-1-carboxylate 5 (600 mg, 31% yield) as a colorless solid. LCMS (ES-): m z 363.43 [M-H]-.

Step-3: To a stirred solution of tert-butyl 4-[4-(2,6-dibenzyloxy-3-pyridyl)-2-hydroxy-phenyl]-3,6-dihydro-2H-pyridine-1-carboxylate 5 (600 mg, 1.06 mmol) in ethanol (10 mL) was purged with N2 for 5 minutes. After, 10% Palladium on carbon (13 mg, 106 mol), PtO2 (12.1 mg, 53.1 mol) were added, and the reaction mixture was stirred under hydrogen (10 psi) at room temperature for 3 hours. Upon completion of the reaction, the reaction mixture was filtered through celite and washed with ethyl acetate (2×20 mL). The filtrate was concentrated under reduced pressure to afford tert-butyl 4-[4-(2,6-dioxo-3-piperidyl)-2-hydroxy-phenyl]piperidine-1-carboxylate 6 (350 mg, 29% yield) as an off-white solid. LCMS (ES-): m z 387.23 [M-H]-.

Step-4: To a stirred solution of tert-butyl 4-[4-(2,6-dioxo-3-piperidyl)-2-hydroxy-phenyl]piperidine-1-carboxylate 6 (350 mg, 901 mol) in tetrahydrofuran (3 mL) were added DBU (336.86 L, 2.3 mmol), (4-acetamidophenyl) (fluorosulfonyl) sulfamoylfluoride (340 mg, 1.1 mmol) and stirred at room temperature for 1 hour. Upon completion, the reaction mixture was concentrated under reduced pressure to afford crude product which was then purified by super fluid chromatography [Column: CHIRALAPK-IH, 30×250 mm, 5 micron, Mobile phase A: 75% CO2, Mobile phase B: 25% (acetonitrile: PA, 1:1), Wavelength: 215 nm]and the fractions containing the compound were combined to afford tert-butyl 4-[4-(2,6-dioxo-3-piperidyl)-2-fluorosulfonyloxy-phenyl]piperidine-1-carboxylate 7 (13.1 mg, 3% yield) as a white solid.

LCMS (ES-): m z 469.32 [M-H]-.

Step-5: To a stirred solution of tert-butyl 4-[4-(2,6-dioxo-3-piperidyl)-2-fluorosulfonyloxy-phenyl]piperidine-1-carboxylate 7 (20 mg, 42.5 mol) in dichloromethane (1 mL) at 0° C. was added trifluoroacetic acid (200 pL, 2.6 mmol,) and stirred at room temperature for 2 hours. 5 Upon completion, the reaction mixture was concentrated under reduced pressure to afford the crude product. The crude product was triturated with diethyl ether (5 mL), dried under reduced pressure to afford the desired product which was lyophilized to afford 3-[3-fluorosulfonyloxy-4-(4-piperidyl)phenyl]-2,6-dioxo-piperidine (A-32) (13.9 mg, 67% yield). LCMS (ES+): m z 371.16 [M+H]+. THNMR (400 Hz DMSO) 6: 10.91 (s, 1H), 8.63 (d, J=9.2 Hz, 1H), 8.30 (d, J=10.0 Hz, 1H), 7.51 (s, 1H), 7.44 (s, 1H), 3.98-4.03 (m, 1H), 3.38-3.45 (m, 2H), 3.11-3.20 (m, 3H), 2.69-2.73 (m, 1H), 2.55-2.56 (m, 1H), 2.22-2.31 (m, 1H), 2.00-2.04 (m, 1H), 1.83-1.88 (m, 4H).

Synthesis A33: Synthesis of 4-((2,4-dioxo-3-azabicyclo[3.1.1]heptan-1-yl)amino)benzenesulfonyl fluoride (A-33)

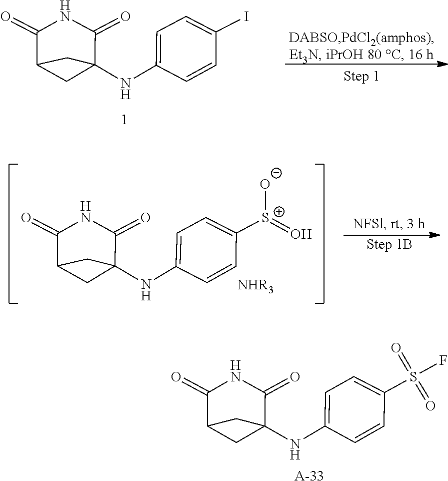

Step 1 and Step 1B: To a stirred solution of (4-iodoanilino)-3-azabicyclo [3.1.1]heptane-2, 4-dione 1 (0.3 g, 877 mol), DABSO (126 mg, 526 mol) in anhydrous isopropanol (6 mL) was added triethyl amine (367 L, 2.6 mmol) and purged with N2 for 5 minutes. After, Pd(amphos)C12 (31 mg, 43.8 mol) was added, and the reaction mixture was stirred at 80° C. for 16 hours. The reaction mixture was cooled to room temperature and N-fluorobenzenesulfonimide (415 mg, 1.3 mmol) was added and the reaction was stirred at room temperature for 3 hours. Upon completion of the reaction, the reaction mixture was diluted with saturated sodium thiosulfate (10 mL) and extracted with ethyl acetate (2×25 mL). The organic layer was dried over sodium sulfate, filtered over a plug of cotton and concentrated under reduced pressure to afford crude product, which was purified by preparative HPLC [Column: X-BRIDGE C18, 19×250 mm, 5 micron, Mobile phase A: 0.1% formic acid in water, Mobile phase B: Acetonitrile, Wavelength: 215 nm]and the fractions containing the compound were combined and lyophilized to afford the desired product 4-[(2,4-dioxo-3-azabicyclo [3.1.1]heptan-1-yl)amino]benzenesulfonyl fluoride (A-33) (44.2 mg, 17% yield) as an off-white solid. LCMS (ES-): m/z 297.08 [M-H]-.

¹H NMR (400 MHz, DMSO-d6) δ: 10.89 (s, 1H), 7.82 (s, 1H), 7.70 (d, J=8.8 Hz, 2H), 6.67 (d, J=8.8 Hz, 2H), 3.01 (t, J=6.6 Hz, 1H), 2.88-2.85 (m, 2H), 2.59-2.55 (m, 2H).

Synthesis A34: Synthesis of 4-(2,4-dioxo-3-azabicyclo[3.1.1]heptan-1-yl)benzenesulfonyl 10 fluoride (A-34)

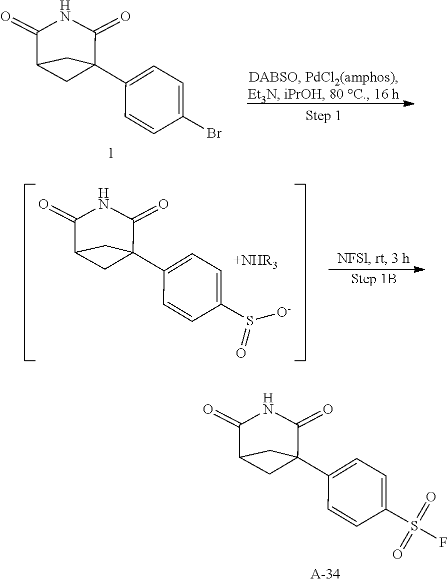

Step 1 and Step 1B: To a stirred solution of 1-(4-bromophenyl)-3-azabicyclo [3.1.1]heptane-2, 4-dione 1 15 (0.3 g, 1.1 mmol), DABSO (154 mg, 643 mol) in anhydrous isopropanol (6 mL) was added triethyl amine (447.82 pL, 3.21 mmol) and purged with N2 for 5 minutes. After, Pd(amphos)C12 (38 mg, 54 mol) was added, and the reaction mixture was stirred at 80° C. for 16 hours. The reaction mixture was cooled to room temperature and N-Fluorobenzenesulfonimide (507 mg, 1.6 mmol) was added, and the reaction mixture was stirred at room temperature for 3 hours. 20 Upon completion of the reaction, the reaction mixture was diluted with saturated aqueous sodium thiosulfate (10 mL) and extracted with ethyl acetate (2×25 mL). The organic layer was, dried over sodium sulfate, filtered over a plug of cotton and concentrated under reduced pressure to afford crude product, which was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product -(2,4-dioxo-3-azabicyclo[3.1.1]heptan-1-yl)benzenesulfonyl fluoride (A-34) (38 mg, 12.52% yield) as an off-white solid. LCMS (ES-): m z 282.03 [M-H]-. ¹H NMR (400 MHz, DMSO-d6) δ: 10.96 (s, 1H), 8.14 (d, J=7.6 Hz, 2H), 7.54 (d, J=8.4 Hz, 2H), 2.99 (t, J=6.0 Hz, 1H), 2.90-2.87 (m, 2H), 2.78-2.74 (m, 2H).

Synthesis A35: Synthesis of 5-(2,4-dioxotetrahydropyrimidin-1(2H)-yl)-2-(piperidin-4-yl)phenyl sulfurofluoridate (A-35) O

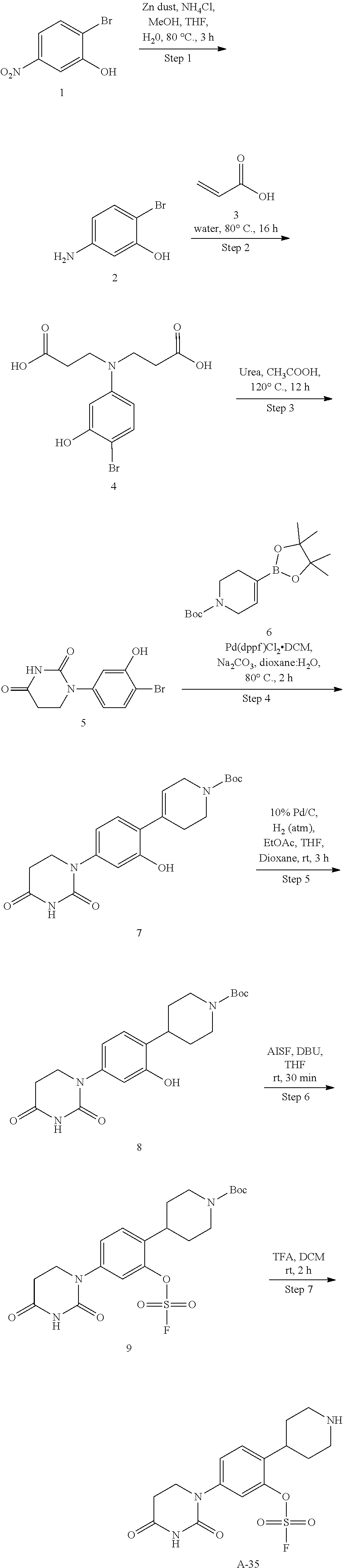

Step-1: To a stirred solution of 2-bromo-5-nitro-phenol 1 (25 g, 115 mmol) in methanol (49 mL), tetrahydrofuran (49 mL) and water (49 mL) were added Zn dust (2.7 g, 115 mmol) and ammonium chloride (6.1 g, 115 mmol) at 0° C. and stirred at 80° C. for 3 hours. Upon completion, the reaction mixture was diluted with water (50 mL) extracted with ethyl acetate (2×100 mL), the combined organic layer was dried over sodium sulfate and concentrated under reduced pressure to afford the crude product. The crude product was triturated with dichloromethane (2×20 mL) and dried under reduced pressure to afford 5-amino-2-bromo-phenol 2 (1.5 g, 52% yield) as a brown solid. LCMS (ES*): m z 187.93 [M+H]+. Step-2: To a stirred solution of 5-amino-2-bromo-phenol 2 (1.56 g, 8.3 mmol) in water (12 mL) was added acrylic acid 3 (1.25 mL, 18.3 mmol) at room temperature and the reaction mixture was heated at 80° C. for 16 hours. Upon completion of the reaction, the reaction mixture diluted with water (30 mL) and extracted with ethyl acetate (2×100 mL). The combined organic layer was washed with saturated sodium chloride solution (20 mL), dried over anhydrous sodium sulfate and concentrated under reduced pressure to afford the crude product 3-[4-bromo-N-(2-carboxyethyl)-3-hydroxy-anilino]propanoic acid 4 (1.52 g, 12% yield) as a solid. LCMS (ES+): m z 332.062 [M+H]+.

Step-3: To a stirred solution of 3-[4-bromo-N-(2-carboxyethyl)-3-hydroxy-anilino]propanoic acid 4 (1.52 g, 4.6 mmol) in acetic acid (14.7 mL) added urea (413 mg, 6.7 mmol) and stirred at 120° C. for 12 hours. After, 10% HCl (0.45 mL, 4.6 mmol) was added to reaction mixture and the reaction was stirred at 120° C. for 12 hours. Upon completion of the reaction, the reaction mixture was diluted with water (50 mL) and extracted with dichloromethane (2×100 mL). The combined organic layer was washed saturated sodium chloride solution (50 mL), dried over anhydrous sodium sulfate and concentrated under reduced pressure to obtain crude product 1-(4-bromo-3-hydroxy-phenyl) hexahydropyrimidine-2, 4-dione 5 (0.8 g, 31% yield) as brown color solid. LCMS (ES+): m z 285.07 [M+H]*.

Step-4: To a stirred solution of 1-(4-bromo-3-hydroxy-phenyl) hexahydropyrimidine-2, 4-dione 5 (400 mg, 1.40 mmol), tert-butyl 4-(4,4,5,5-tetramethyl-1,3,2-dioxaborolan-2-yl)-3,6-dihydro-2H-pyridine-1-carboxylate 6 (434 mg, 1.40 mmol) in dioxane (8.0 mL) and water (2 mL) was added Na2CO3 (372 mg, 3.5 mmol). The reaction mixture was de-gassed with argon gas for 15 minutes, after which [1,1′-bis(diphenylphosphino)ferrocene]dichloropalladium(II) complex with dichloromethane (92 mg, 112 mol) was added and the reaction was stirred at 80° C. for 2 hours. Upon completion of the reaction, the reaction mixture was filtered through a celite bed and washed with ethyl acetate (2×25 mL). The organic layer was concentrated under reduced pressure to afford the crude product, which was purified by flash column chromatography (200-430 mesh silica gel; using 80% of ethyl acetate in pet-ether as eluent) to afford desired product tert-butyl 4-[4-(2,4-dioxohexahydropyrimidin-1-yl)-2-hydroxy-phenyl]-3,6-dihydro-2H-pyridine-1-carboxylate 7 (200 mg, 13% yield) as brown solid. LCMS (ES-): m z 386.23 [M-H]-.

Step-5: A stirred solution of tert-butyl 4-[4-(2,4-dioxohexahydropyrimidin-1-yl)-2-hydroxy-phenyl]-3,6-dihydro-2H-pyridine-1-carboxylate 7 (200 mg, 516 mol) in ethanol (2 mL) was degassed with N2 for 10 minutes, after which 10% Palladium on carbon, (28 mg, 258 mol) and PtO2 (11.72 mg, 51.62 mol, 1.15 L) was added. The reaction mixture was stirred under H2 (10 psi) at room temperature for 3 hours. Upon completion of the reaction, the reaction mixture was filtered through celite bed and washed with ethyl acetate (30 mL). The filtrate was concentrated under reduced pressure to afford the crude product tert-butyl 4-[4-(2, 4-dioxohexahydropyrimidin-1-yl)-2-hydroxy-phenyl]piperidine-1-carboxylate 8 (200 mg, 51.63% yield) as a brown solid. LCMS (ES-): m z 388.22 [M-H]-.

Step-6: To a stirred solution of tert-butyl 4-[4-(2,4-dioxohexahydropyrimidin-1-yl)-2-hydroxy-phenyl]piperidine-1-carboxylate 8 (200 mg, 514 mol) in tetrahydrofuran (4.9 mL) was added (4-Acetamidophenyl)(fluorosulfonyl)sulfamoyl fluoride (194 mg, 616 mol) and 2,3,4,6,7,8,9,10-octahydropyrimido[1,2-a]azepine (192 L, 1.3 mmol,) and stirred at room temperature for 30 minutes. Upon completion of the reaction, the reaction mixture was quenched with water (20 mL) and extracted with ethyl acetate (2×50 mL). The combined organic layer was dried over sodium sulfate and concentrated under reduced pressure to get the crude product tert-butyl 4-[4-(2, 4-dioxohexahydropyrimidin-1-yl)-2-fluorosulfonyloxy-phenyl]piperidine-1-carboxylate 9 (200 mg, 15% yield) as a viscous oil. LCMS (ES-): m z 470.02 [M-H]-.

Step-7: To a stirred solution of tert-butyl 4-[4-(2,4-dioxohexahydropyrimidin-1-yl)-2-fluorosulfonyloxy-phenyl]piperidine-1-carboxylate 9 (0.2 g, 424 mol) in dichloromethane (3 mL) was added trifluoroacetic acid (390 pL, 5.1 mmol) at 0° C. and stirred at room temperature for 2 hours. Upon completion of the reaction, the reaction mixture was concentrated under reduced pressure to get the crude product, which was purified by preparative HPLC [Column: GEMINI-NX C18, 21×250 mm, 5 micron, Mobile phase A: 0.1% formic acid in water, Mobile phase B: Acetonitrile, Wavelength: 215 nm]and the fractions containing the compound were combined and lyophilized to afford the desired product 1-[3-fluorosulfonyloxy-4-(4-piperidyl)phenyl]-2,4-dioxo-hexahydropyrimidine (A-35) (39.3 mg, 19% yield) as an off-white solid. LCMS (ES*): m z 372.15 [M+H]+. H-NMR (400 MHz, DMSO-d6): δ 10.51 (s, 1H), 8.24 (s, 1H), 7.60 (s, 1H), 7.51 (s, 2H), 3.83 (t, J=6.6 Hz, 2H), 3.32-3.36 (m, 2H), 3.26-3.29 (m, 1H), 3.06-3.11 (m, 2H), 2.72 (t, J=6.6 Hz, 2H), 1.78-1.80 (m, 4H).

Synthesis A36: Synthesis of 5-(2,6-dioxopiperidin-3-yl)-2-(piperazin-1-yl)phenyl sulfurofluoridate (A-36)

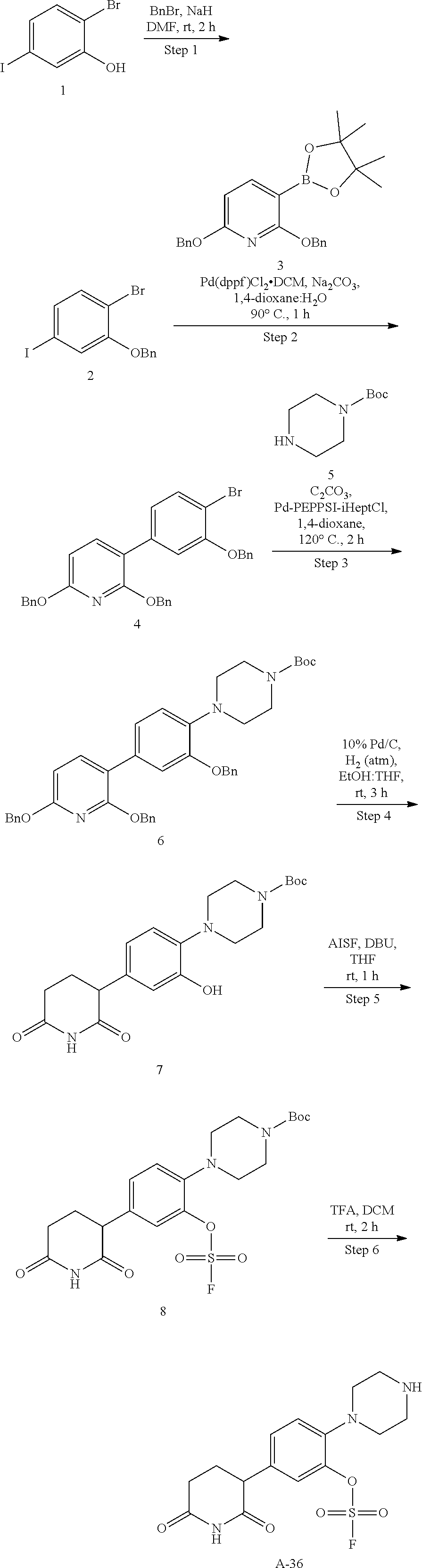

Step-1:

To a stirred solution of 2-bromophenol 1 (2.7 mL, 23.1 mmol) in dimethylformamide (40 mL) was added NaH (50% dispersion in oil) (2.2 g, 46.2 mmol) in dimethylformamide (40 5 mL) at 0° C. and stirred for 15 minutes at room temperature. After, benzyl bromide (2.75 mL, 23.1 mmol) was added, and the reaction was stirred at room temperature for 2 hours. Upon completion of the reaction, the reaction mixture was quenched with ice chilled water and extracted with ethyl acetate (2×25 mL). The organic layer was dried over anhydrous sodium sulfate and concentrated under reduced pressure to afford crude product. The crude product was purified by column chromatography (Davisil silica; using ~5% ethyl acetate in petroleum ether as an eluent) to afford 2-benzyloxy-1-bromo-4-iodo-benzene 2 (3.75 g, 39% yield).

LCMS (ES-): m z 386.89 [M-H]-.

Step-2: To a stirred solution of 2-benzyloxy-1-bromo-4-iodo-benzene 2 (3.75 g, 9.6 mmol), 2,6-dibenzyloxy-3-(4,4,5,5-tetramethyl-1,3,2-dioxaborolan-2-yl)pyridine 3 (6.0 g, 14.5 mmol) in a mixture of 1,4-dioxane (40 mL), water (8 mL) was added sodium carbonate (1.2 mL, 29 mmol) and purged with N2 for 10 minutes. After, [1,1′-bis(diphenylphosphino)ferrocene]dichloropalladium(II) complex with dichloromethane (787 mg, 964 mol) was added and the reaction was stirred at 90° C. for 1 hour. Upon completion of 20 the reaction, the reaction was filtered through celite and washed with ethyl acetate (2×25 mL).

The filtrate was concentrated under reduced pressure to afford the crude product which was purified by column chromatography (Davisil silica, using 2% ethyl acetate in petroleum ether as an eluent) to afford 2,6-dibenzyloxy-3-(3-benzyloxy-4-bromo-phenyl)pyridine 4 (2.7 g, 21% yield). LCMS (ES*): m z 552.19 [M+H]+. 25 Step-3: To a stirred solution of 2,6-dibenzyloxy-3-(3-benzyloxy-4-bromo-phenyl) 4 (1.2 g, 2.2 mmol), tert-butyl piperazine-1-carboxylate 5 (809 mg, 4.3 mmol) in 1,4-dioxane (20 mL) added cesium carbonate (2.12 g, 6.5 mmol) and purged with N2 for 10 minutes. After, Pd-PEPPSI-iHeptCl (212 mg, 217 mol) was added and the reaction mixture was stirred at 120° C. for 2 hours. Upon completion, the reaction mixture was filtered through a celite bed and washed with ethyl acetate (2×25 mL). The filtrate was concentrated under reduced pressure to afford crude product which was purified by column chromatography (Davisil silica; using 20% ethyl acetate in petroleum ether as an eluent) to afford tert-butyl 4-[2-benzyloxy-4-(2,6-dibenzyloxy-3-pyridyl)phenyl]piperazine-1-carboxylate 6 (360 mg, 20% yield). LCMS (ES*): m z 658.48 [M+H]+.

Step-4: A stirred solution of tert-butyl 4-[2-benzyloxy-4-(2,6-dibenzyloxy-3-pyridyl)phenyl]piperazine-1-carboxylate 6 (300 mg, 456 mol) in a mixture of ethyl acetate (5 mL), tetrahydrofuran (5 mL) was degassed with N2 for 10 minutes. After 10% Pd/C (300 mg, 797 mol) was added and the reaction was stirred under H2 (10 psi) at room temperature for 3 hours. After completion of the reaction, the reaction mixture was concentrated under reduced pressure to afford crude product tert-butyl 4-[4-(2,6-dioxo-3-piperidyl)-2-hydroxy-phenyl]piperazine-1-carboxylate 7 (140 mg, 61% yield). LCMS (ES*): m z 390.53 [M+H]*. Step-5: To a stirred solution of tert-butyl 4-[4-(2,6-dioxo-3-piperidyl)-2-hydroxy-phenyl]piperazine-1-carboxylate 7 (140 mg, 359 mol) in tetrahydrofuran (870 pL) was added DBU (134 L, 899 mol) and (4-acetamidophenyl)(fluorosulfonyl)sulfamoyl fluoride (136 mg, 431 mol) and stirred at room temperature for 1 hour. After the completion of the reaction, the reaction mixture was diluted with water (15 mL) and extracted with ethyl acetate (2×15 mL). The organic layer was dried over anhydrous sodium sulfate and concentrated under reduced pressure to afford the crude product. The crude product was purified by preparative HPLC [Column: GEMINI-NX C18, 21×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[4-(2,6-dioxo-3-piperidyl)-2-fluorosulfonyloxy-phenyl]piperazine-1-carboxylate 8 (35 mg, 19% yield) as an off-white solid. LCMS (ES-): m z 470.16 [M-H]-.

Step-6: To a stirred solution of tert-butyl 4-[4-(2,6-dioxo-3-piperidyl)-2-fluorosulfonyloxy-phenyl]piperazine-1-carboxylate 8 (35 mg, 74.2 mol) in dichloromethane (2.0 mL) added trifluoroacetic acid (300 L, 3.9 mmol) at 0° C. and stirred at room temperature for 2 hours.

After the completion of the reaction, the reaction mixture was concentrated under reduced pressure to and triturated with diethyl ether (2×5 mL), dried and lyophilised to afford 1-[4-(2,6-dioxo-3-piperidyl)-2-fluorosulfonyloxy-phenyl]piperazine (A-36) (24 mg, 64% yield). LCMS (ES*): m z 372.07 [M+H]+. ¹H NMR (400 MHz, DMSO-d6) δ: 10.87 (s, 1H), 8.74 (s, 2H), 7.48 (s, 1H), 7.34 (t, J=10 Hz, 2H), 3.94 (dd, J=4.8 Hz, J=12 Hz, 1H), 3.22-3.19 (m, 8H), 2.71-2.63 (m, 1H), 2.54-2.53 (s, 1H), 2.31-2.21 (s, 1H).

Synthesis A37: Synthesis of 4-(2,4-dioxo-3-azabicyclo[3.1.1]heptan-1-yl)phenyl sulfurofluoridate (A-37)

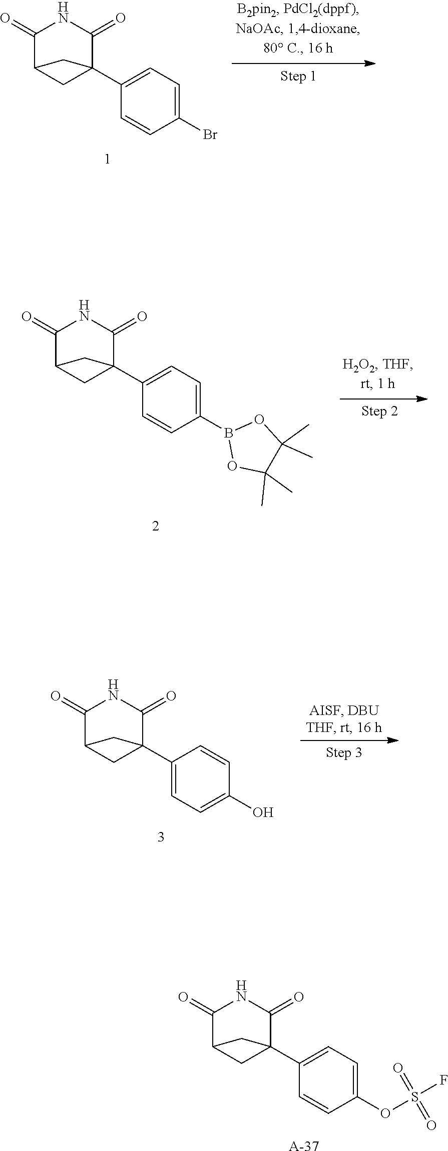 [SMILES: O=C1NC(=O)C2(c3ccc(Br)cc3)CC1C2 ; CC1(C)OB(c2ccc(C34CC(C3)C(=O)NC4=O)cc2)OC1(C)C ; O=C1NC(=O)C2(c3ccc(O)cc3)CC1C2 ; O=C1NC(=O)C2(c3ccc(OS(=O)(=O)F)cc3)CC1C2 ; "A-37"]

Step-1: To a solution of 1-(4-bromophenyl)-3-azabicyclo[3.1.1]heptane-2,4-dione 1 (0.45 g, 1.6 mmol), Bis(pinacolato)diboron (612 mg, 2.4 mmol) in 1,4-dioxane (10 mL) was added potassium acetate (473 mg, 4.8 mmol) at room temperature and degassed with argon gas for 10 minutes. After, Pd(dppf)Cl2 (117 mg, 0.16 mmol) was added, and the reaction was stirred at 80° C. for 16 hours. Upon completion, the reaction mixture was diluted with water (30 mL) 15 and extracted with ethyl acetate (3×50 mL). The combined organic layer was dried over anhydrous sodium sulfate and concentrated under reduced pressure to obtain crude product, 1-[4-(4,4,5,5-tetramethyl-1,3,2-dioxaborolan-2-yl)phenyl]-3-azabicyclo[3.1.1]heptane-2,4-dione 2 (0.45 g, 65% yield) as a brown solid. LCMS (ES+): m z 328.22 [M+H]*.

Step-2: To a stirred solution of 1-[4-(4,4,5,5-tetramethyl-1,3,2-dioxaborolan-2-yl)phenyl]-3-azabicyclo[3.1.1]heptane-2,4-dione 2 (0.35 g, 1.1 mmol) in tetrahydrofuran (35 mL) was added 35% hydrogen peroxide (2.1 mL, 68.5 mmol) at 0° C. and stirred at room temperature for 1 hour. Upon completion, the reaction mixture was diluted with water (25 mL) and extracted with ethyl acetate (3×25 mL). The combined organic layer was dried over anhydrous sodium 25 sulfate and concentrated under reduced pressure to afford crude product 1-(4-hydroxyphenyl)-3-azabicyclo[3.1.1]heptane-2,4-dione 3 (0.30 g, 40% yield). This material was used crude in the next step. LCMS (ES-): m z 218.15 [M+H]*.

Step-3: To a stirred solution of 1-(4-hydroxyphenyl)-3-azabicyclo[3.1.1]heptane-2,4-dione 3 (0.350 g, 1.6 mmol) in tetrahydrofuran (20 mL) was added DBU (602 L, 4.03 mmol), (4-acetamidophenyl)(fluorosulfonyl)sulfamoyl fluoride (608 mg, 1.92 mmol) at 0° C. and stirred at 25° C. for 16 hours. After completion of the reaction, the reaction mixture was concentrated under reduced pressure to obtain crude product, which was purified by preparative HPLC [Column: X bridge C18, 19×150 mm, 5 micron, Mobile phase A: 0.1% formic acid in water, Mobile phase B: Acetonitrile, Wavelength: 215 nm]and the fractions containing the compound were combined and lyophilized to afford the desired product 1-(4-fluorosulfonyloxyphenyl)-2,4-dioxo-3-azabicyclo[3.1.1]heptane (A-37) (0.018 g, 4% yield) as an off-white solid. LCMS (ES+): 298.307 [M+H]+. H-NMR (400 MHz, DMSO-d6): δ 10.88 (s, 1H), 7.59 (d, J=8.40 Hz, 2H), 7.33 (d, J=8.40 Hz, 2H), 2.97 (t, J=6.00 Hz, 1H), 2.83-2.80 (m, 2H), 2.72-2.71 (m, 2H).

Synthesis A38: Synthesis of 5-((2,6-dioxopiperidin-3-yl)oxy)-2-(piperidin-4-yl)benzenesulfonyl fluoride (A-38)

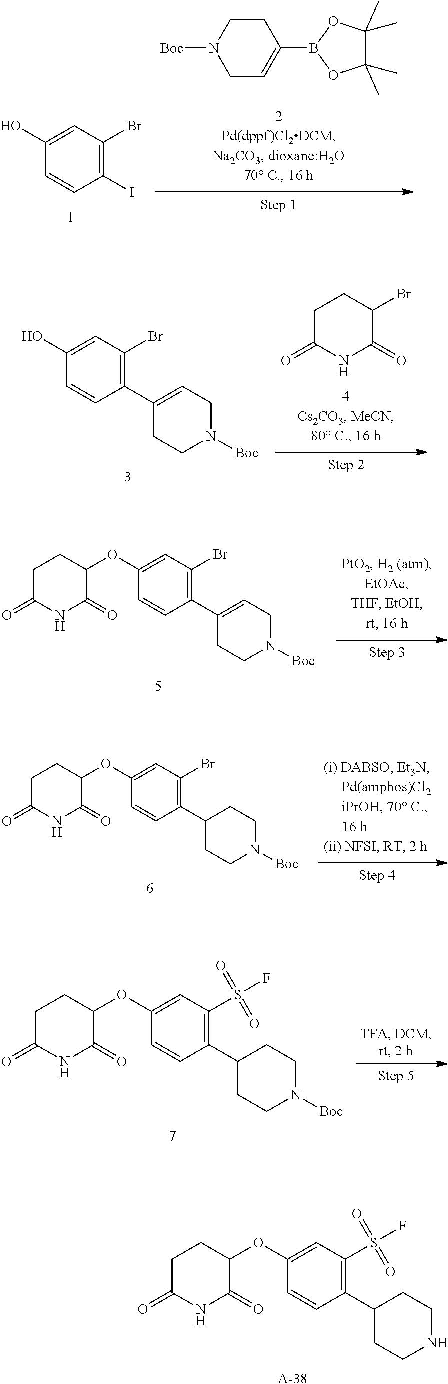

To a stirred solution of 3-bromo-4-iodo-phenol 1 (3 g, 10.0 mmol) and tert-butyl 4-(4,4,5,5-tetramethyl-1,3,2-dioxaborolan-2-yl)-3,6-dihydro-2H-pyridine-1-carboxylate 2 (4.03 5 g, 13.1 mmol) in a mixture of 1,4-dixoane (25 mL), water (5 mL) was added potassium carbonate (2.77 g, 20.1 mmol) and purged with N2 (1 atm) for 10 minutes. After, Pd(dppf)C12 DCM (0.40 g, 0.50 mmol) was added, and the reaction mixture was stirred at 70° C. for 16 hours. Upon completion of the reaction, the reaction mixture was diluted with water (200 ml) and extracted with ethyl acetate (2×250 ml). The organic layer was dried over 10 anhydrous sodium sulfate, filtered and evaporated under reduced pressure to afford the crude product. The crude product was purified by comb flash (Davisil silica, using ~20% ethyl acetate and petroleum ether as an eluent) to afford tert-butyl 4-(2-bromo-4-hydroxy-phenyl)-3,6-dihydro-2H-pyridine-1-carboxylate 3 (2.5 g, 67% yield) as yellow solid.¹H NMR (400 MHz, DMSO-d6) δ: 7.08 (d, J=2.4 Hz, 1H), 7.02 (d, J=8.0 Hz, 1H), 6.75 (dd, J=8.2, 2.6 Hz, 1H), 15 5.59 (s, 1H), 5.13 (s, 1H), 4.03 (s, 2H), 3.71 (t, J=6.2 Hz, 1H), 3.60 (t, J=5.6 Hz, 1H), 2.43 (t, J=6.2 Hz, 2H), 1.50 (s, 9H).

Step-2: To a stirred solution of tert-butyl 4-(2-bromo-4-hydroxy-phenyl)-3,6-dihydro-2H-pyridine-1-carboxylate 3 (2 g, 5.7 mmol), 3-bromopiperidine-2,6-dione 4 (3.25 g, 16.9 mmol) 20 in acetonitrile (25 mL) was added cesium carbonate (5.52 g, 16.9 mmol) and stirred at 80° C. for 16 hours. Upon completion of the reaction, the reaction mixture was filtered through celite bed and washed with acetonitrile (50 mL). The filtrate was evaporated under reduced pressure to afford the crude product. The crude product was purified by column chromatography (Davisil silica, 40% ethyl acetate and petroleum ether as an eluent) to afford tert-butyl 4-[2-bromo-4-[(2,6-dioxo-3-piperidyl)oxy]phenyl]-3,6-dihydro-2H-pyridine-1-carboxylate 5 (800 mg, 30% yield) as orange solid LCMS (ES-): m z 462.78 [M-H]-.

Step-3: To a stirred solution of tert-butyl 4-[2-bromo-4-[(2,6-dioxo-3-piperidyl)oxy]phenyl]-3,6-dihydro-2H-pyridine-1-carboxylate 5 (0.40 g, 0.85 mmol) in tetrahydrofuran (10 mL), ethyl acetate (10 mL) and ethanol (2 mL) was added PtO2 (0.39 g, 1.7 mmol) and stirred under H2 (10 psi) at room temperature for 16 hours. Upon completion of the reaction, the reaction mixture was filtered through celite bed using ethyl acetate (200 ml). The filtrate was concentrated under reduced pressure to afford crude product. The crude product was purified by column chromatography (Davisil silica, 50% ethyl acetate and petroleum ether as an eluent) to afford tert-butyl 4-[2-bromo-4-[(2,6-dioxo-3-piperidyl)oxy]phenyl]piperidine-1-carboxylate 6 (300 mg, 64% yield) as an off-white solid. LCMS (ES-): m z 465.24 [M-H]-.

Step-4: To a tert-butyl 4-[2-bromo-4-[(2,6-dioxo-3-piperidyl)oxy]phenyl]piperidine-1-carboxylate 6 (0.30 g, 0.64 mmol), 1,4-diazabicyclo[2.2.2]octane bis(sulfur dioxide) adduct (0.20 g, 0.83 mmol) in isopropyl alcohol (6 mL) added triethylamine (0.26 ml, 1.9 mmol) and purged with N2 (1 atm) for 5 minutes. After, Pd(amphos)C12(0.045 g, 0.064 mmol) was added, and the reaction mixture was stirred at 70° C. for 16 hours. The reaction mixture was cooled to room temperature, and N-fluorobenzenesulfonimide (0.30 g, 0.96 mmol) was added, and the reaction mixture was stirred at room temperature for 3 hours. Upon completion of the reaction, the reaction mixture was diluted with water (50 mL) and extracted with ethyl acetate (2×100 mL). The combined organic layer was dried over anhydrous sodium sulfate filtered, evaporated under reduced pressure to obtain tert-butyl 4-[4-[(2,6-dioxo-3-piperidyl)oxy]-2-fluorosulfonyl-phenyl]piperidine-1-carboxylate 7 (230 mg, 31% yield) as an off-white solid.

LCMS (ES-): m z 469.13 [M-H]-.

Step-5: To a stirred solution of tert-butyl 4-[4-[(2,6-dioxo-3-piperidyl)oxy]-2-fluorosulfonyl-phenyl]piperidine-1-carboxylate 7 (200 mg, 425 mol) in dichloromethane (5 mL) was added trifluoroacetic acid (65.1 pL, 850.1 mol) dropwise at 0° C. and stirred at room temperature for 2 hours. Upon completion of the reaction, the reaction mixture was evaporated under reduced pressure to afford crude product. The crude was purified by preparative HPLC [Column: X-Select C18, 19×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 5-[(2,6-dioxo-3-piperidyl) oxy]-2-(4-piperidyl) (A-38) H-NMR (400 MHz, DMSO) 6: 11.02 (s, 1H), 0.92 (s, 1H), 7.68 (s, 1H), 7.65 (d, J=2.8 Hz, 1H), 7.59 (dd, J=8.6, 2.6 Hz, 1H), 5.44-5.40 (m, 1H), 3.43-3.40 (m, 3H), 3.09-3.02 (m, 2H), 2.78-2.64 (m, 2H), 2.29-2.22 (m, 2H), 1.97-1.94 (m, 4H). LCMS (ES*): m/z 371.34 [M+H]+.

Synthesis A39: Synthesis of 5-(2,4-dioxotetrahydropyrimidin-1(2H)-yl)-2-(piperazin-1-yl)phenyl sulfurofluoridate (A-39)

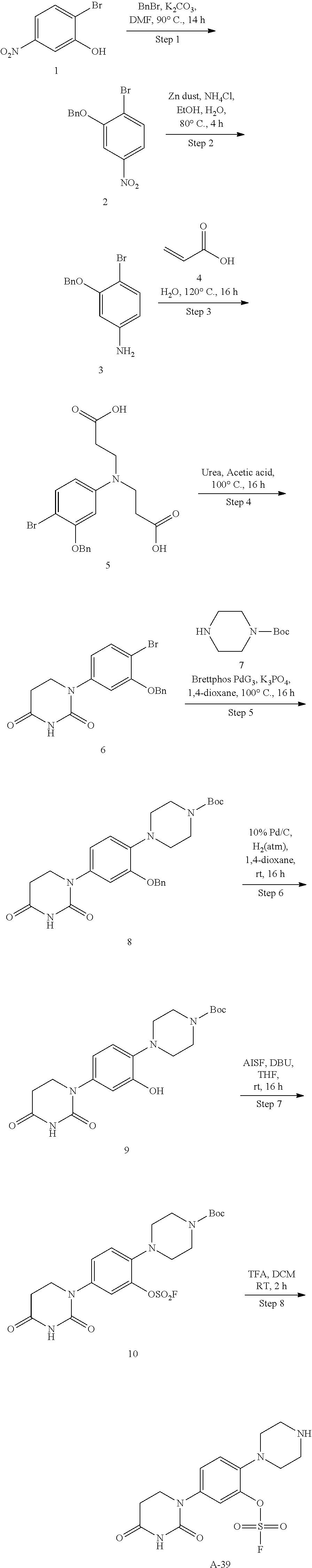

To the stirred solution of 2-bromo-5-nitro-phenol 1 (5.0 g, 22.9 mmol) in ethanol (100 10 mL) added potassium carbonate (3.80 g, 27.5 mmol), potassium iodide (1.90 g, 11.5 mmol) and benzyl bromide (3.6 mL, 29.8 mmol) consecutively at room temperature. Upon addition, the reaction mixture was heated to 90° C. for 14 hours. Upon completion of the reaction, the solvent was removed under reduced pressure and the residue was diluted with saturated ammonium chloride (100 mL) and extracted with ethyl acetate (2×100 mL). The combined organic layers were dried over anhydrous sodium sulfate and evaporated under reduced pressure to obtain crude product. The crude was purified by flash chromatography (100-200 mesh silica gel, 20% ethyl acetate and petroleum ether as eluent) to afford 2-benzyloxy-1-bromo-4-nitro-benzene 2 (6.5 g, 90% yield) as a pale-yellow solid. LCMS (ES*): m/z 307.99 [M+H]+.

Step-2: To a stirred solution of 2-benzyloxy-1-bromo-4-nitro-benzene 2 (4.0 g, 13.0 mmol) in a mixture of ethanol (200 mL), water (40 mL) were added zinc (8.49 g, 129.8 mmol) and ammonium chloride (6.94 g, 129.8 mmol) at room temperature and stirred at 80° C. for 4 hours.

Upon completion of the reaction, the reaction mixture was passed through celite bed and washed with ethyl acetate (100 mL). The filtrate was diluted with water (100 mL) and extracted with ethyl acetate (3×100 mL). The combined organic layer was dried over anhydrous sodium sulfate and concentrated under reduced pressure to obtain the 3-benzyloxy-4-bromo-aniline 3 (3.2 g, 79% yield) as a pale-yellow solid. LCMS (ESI-) m/z 280.00 [M+2H]+.

Step-3: To the stirred solution of 3-benzyloxy-4-bromo-aniline 3 (3.2 g, 11.5 mmol) in water (50 mL) added acrylic acid 4 (4.7 mL, 69.03 mmol) and stirred at 120° C. for 16 hours. Upon completion of the reaction, the mixture was diluted with water (100 mL) and extracted with ethyl acetate (2×100 mL). The combined organic layer was dried over anhydrous sodium sulfate and concentrated under reduced pressure to afford the crude product, which was purified by column chromatography (230-400 mesh silica gel, using 65% ethyl acetate in petroleum ether as an eluent) to afford 3-[3-benzyloxy-4-bromo-N-(2-carboxyethyl)anilino]propanoic acid 5 (1.8 g, 27% yield) as an off-white solid. LCMS (ES+): m z 422.30 [M+H]+.

Step-4: To a stirred solution of 3-[3-benzyloxy-4-bromo-N-(2-carboxyethyl)anilino]propanoic acid 5 (1.8 g, 4.3 mmol) in acetic acid (50 mL) was added urea (1.9 mL, 42.6 mmol) and stirred at 100° C. for 16 hours. After, 10% aqueous HCl (20 mL) was added, and the reaction mixture was heated to 100° C. for 1 hour. Upon completion of the reaction, the reaction mixture was diluted with water (100 mL) and extracted with ethyl acetate (2×100 mL). The combined organic layer was dried over anhydrous sodium sulfate and evaporated under reduced pressure to obtain crude product, which was purified by column chromatography (230-400 mesh silica gel, using 70% ethyl acetate in petroleum ether as an eluent) to afford 1-(3-benzyloxy-4-bromo-phenyl)hexahydropyrimidine-2,4-dione 6 (837 mg, 40% yield) as an off-white solid LCMS (ES+): m z 377.09 [M+H]+. 5 Step-5: To a stirred solution of 1-(3-benzyloxy-4-bromo-phenyl)hexahydropyrimidine-2,4-dione 6 (400 mg, 1.1 mmol) and tert-butyl piperazine-1-carboxylate 7 (397 mg, 2.1 mmol) in 1,4-dioxane (8 mL) was added cesium carbonate (1.04 g, 3.2 mmol) in sealed tube at room temperature. The reaction mixture was degassed for 10 minutes with Argon before adding Pd-PEPPSI-iHeptCl (104 mg, 107 mol) and stirred at 100° C. for 16 hours. Upon completion of the reaction, the reaction mixture was filtered through celite bed and washed with ethyl acetate (2×10 mL). The combined organic layer was dried over anhydrous sodium sulfate and concentrated under reduced pressure to obtain tert-butyl 4-[2-benzyloxy-4-(2,4-dioxohexahydropyrimidin-1-yl)phenyl]piperazine-1-carboxylate 8 (200 mg, 27% yield) as viscous brown liquid. LCMS (ES+): m/z 481.48 [M+H]+.

Step-6: To the stirred solution of tert-butyl 4-[2-benzyloxy-4-(2,4-dioxohexahydropyrimidin-1-yl)phenyl]piperazine-1-carboxylate 8 (200 mg, 416 mol) in ethyl acetate (5.0 mL), tetrahydrofuran (5.0 mL) was added 10% Palladium on activated charcoal (200 g, 1.9 mol) and stirred under H2 (50 psi) in a 250 mL par shaker, at room temperature for 16 hours. Upon completion, the reaction mixture was filtered over a celite pad and washed with ethyl acetate (2×25 mL). The filtrate was concentrated under reduced pressure to obtain tert-butyl 4-[4-(2, 4-dioxohexahydropyrimidin-1-yl)-2-hydroxy-phenyl]piperazine-1-carboxylate 9 (0.18 g, 59% yield) as greasy solid. LCMS (ES-): m z 389.225 [M-H]-.

Step-7: To a stirred solution tert-butyl 4-[4-(2, 4-dioxohexahydropyrimidin-1-yl)-2-hydroxy-phenyl]piperazine-1-carboxylate 9 (180 mg, 461 mol) in tetrahydrofuran (177 mL) was added in single neck round bottom flask at room temperature. To the reaction mixture (4-acetamidophenyl)(fluorosulfonyl)sulfamoyl fluoride (174 mg, 553 mol) and 2,3,4,6,7,8,9,10-octahydropyrimido[1,2-a]azepine (172 pL, 1.2 mmol) was added into the reaction mixture and stirred at room temperature for 2 hours. On completion, reaction mixture was filtered through celite bed and washed with ethyl acetate (3×5 mL). The organic layer was washed with saturated sodium chloride solution (2×5 mL), dried over anhydrous sodium sulfate and concentrated under reduced pressure to get crude product tert-butyl 4-[4-(2,4-dioxohexahydropyrimidin-1-yl)-2-fluorosulfonyloxy-phenyl]piperazine-1-carboxylate 10 (0.20 g, 44% yield) as colorless semi solid. LCMS (ES-): m z 471.28 [M-H]-.

Step-8: To the stirred solution tert-butyl 4-[4-(2,4-dioxohexahydropyrimidin-1-yl)-2-fluorosulfonyloxy-phenyl]piperazine-1-carboxylate 10 (207 mg, 438 mol) in dichloromethane (20 mL) added trifluoroacetic acid (335.28 L, 4.38 mmol) and stirred at room temperature for 2 hours. Upon completion of the reaction, the reaction mixture was concentrated under reduced pressure to get crude product. The crude product was purified by preparative HPLC [Column: UNISIL, 19×250 mm, 5 micron, Mobile phase A: 0.1% 10 trifluoroacetic acid in water, Mobile phase B: acetonitrile, Wavelength: 215 nm]and the fractions containing the compound were combined and lyophilized to afford the desired product 1-(3-fluorosulfonyloxy-4-piperazin-1-yl-phenyl)-2, 4-dioxo-hexahydropyrimidine (A-39) (26 mg, 12% yield) as an off-white solid. LCMS (ES*): m z 373.31 [M+H]+. ¹H NMR (400 MHz), DMSO-d6) δ: 10.47 (s, 1H), 8.73 (s, 1H), 7.62 (d, J=2.00 Hz, 1H), 7.44-7.44 (m, 1H), 7.38-7.40 (m, 1H), 3.80 (t, J=6.40 Hz, 2H), 3.19-3.23 (m, 8H), 2.72 (t, J=6.80 Hz, 2H).

Synthesis A40: Synthesis of 3-(((2,6-dioxopiperidin-3-yl)methyl)amino)phenyl sulfurofluoridate (A-40)

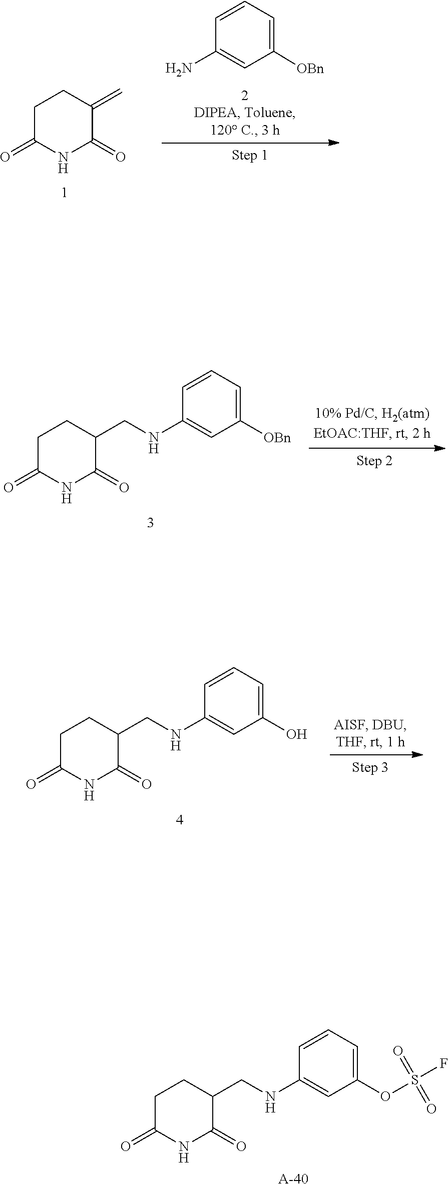

Step-1: To a stirred solution of 3-methylenepiperidine-2,6-dione 1 (0.3 g, 2.4 mmol) and 3-benzyloxyaniline 2 (717 mg, 3.60 mmol) in toluene (10 mL) added N,N-diisopropylethylamine (2.09 mL, 12 mmol) and stirred at 120° C. for 3 hours in microwave. Upon completion, the reaction mixture was concentrated under reduced pressure to afford the crude product. The crude was purified by column chromatography (Devisal silica, using 50% ethyl acetate and petroleum ether as an eluent) to afford product 3-[(3-benzyloxyanilino)methyl]piperidine-2,6-dione 3 (0.41 g, 45% yield). LCMS (ES*): m z 325.18 [M+H]+.

Step-2: To a stirred solution of 3-[(3-benzyloxyanilino)methyl]piperidine-2,6-dione 3 (0.25 g, 771 mol) in ethyl acetate (3 mL), tetrahydrofuran (3 mL) was degassed with N2 for 10 minutes. After, 10% Pd/C (50% wet wt/wt) (0.2 g, 1.9 mmol) and stirred under H2 (10 psi) at room temperature for 2 hours. Upon completion of the reaction, the reaction mixture was concentrated under reduced pressure to afford 3-[(3-hydroxyanilino) methyl]piperidine-2,6-dione 4 (0.13 g, 17.28% yield). LCMS (ES*): m z 235.101 [M+H]+.

Step-3: To a stirred solution of 3-[(3-hydroxyanilino)methyl]piperidine-2,6-dione 4 (0.13 g, 555 mol) in tetrahydrofuran (5 mL) added DBU (208 L, 1.4 mmol), 4-(acetylamino)phenyl]imidodisulfuryl difluoride (209 mg, 666 mol) and stirred at room 15 temperature for 1 hour. Upon completion of the reaction, the reaction mixture was concentrated under reduced pressure to afford the crude product. The crude product was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-[(2,6-dioxo-3-piperidyl) methylamino]benzenesulfonyl fluoride (A-40) (0.026 g, 15% yield) as an off-white solid. LCMS (ES*): m z 317.24 [M+H]+. ¹H NMR (400 MHz, DMSO-d6) δ: 10.74 (s, 1H), 7.23 (t, J=8.8 Hz, 1H), 6.71 (d, J=7.2 Hz, 2H), 6.62 (d, J=9.2 Hz, 1H), 6.35 (t, J=6 Hz, 1H), 3.50-3.46 (m, 1H), 3.33-3.24 (m, 1H), 2.78-2.71 (m, 1H), 2.53 (t, J=4.8 Hz, 2H), 2.02-1.97 (m, 1H), 1.79 (m, 1H).

Synthesis A41: Synthesis of 5-((2,6-dioxopiperidin-3-yl)amino)-2-(piperidin-4-yl)benzenesulfonyl fluoride (A-41)

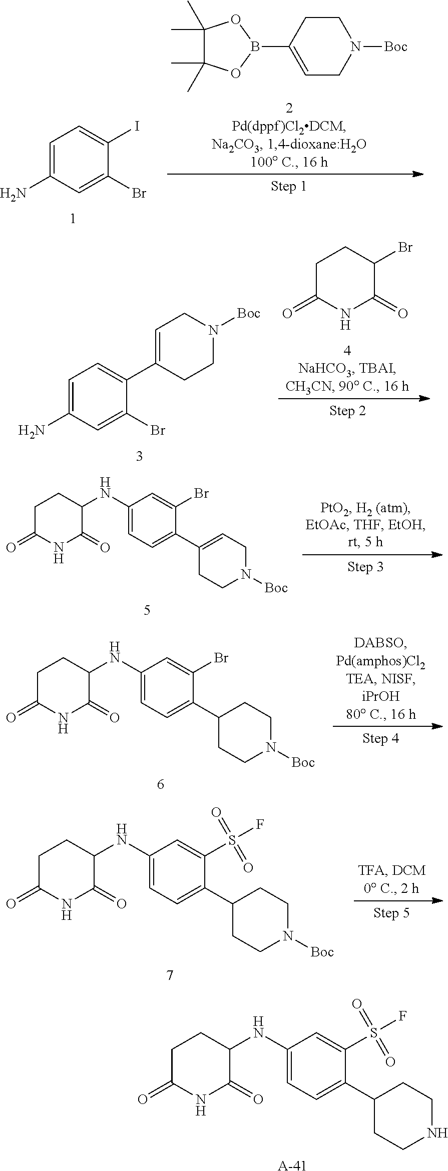

Step-1:

To a stirred solution of 3-bromo-4-iodo-aniline 1 (2 g, 6.7 mmol), tert-butyl 4-(4,4,5,5-tetramethyl-1,3,2-dioxaborolan-2-yl)-3,6-dihydro-2H-pyridine-1-carboxylate 2 (2.1 g, 6.7 mmol) in a mixture of water (0.1 mL), dioxane (10 mL) was added tripotassium phosphate (4.27 g, 20.1 mmol) and purged with N2 for 5 minutes. After, [1,1′-bis(diphenylphosphino)ferrocene]dichloropalladium(II) complex (274 mg, 336 mol) was 10 added at room temperature and the reaction mixture was stirred at 100° C. for 16 hours. After completion of reaction, the reaction mixture was filtered through celite bed and washed with ethyl acetate (2×10 mL). The filtrate was concentrated under reduced pressure to get the crude product, which was purified by column chromatography (Davisil silica; using 18% ethyl acetate in petroleum ether as an eluent) to afford tert-butyl 4-(4-amino-2-bromo-phenyl)-3,6-dihydro-2H-pyridine-1-carboxylate 3 (0.75 g, 29% yield) as an off-white solid. LCMS (ES*): m/z 353.26 [M+H]+.

Step-2: To a stirred solution of tert-butyl 4-(4-amino-2-bromo-phenyl)-3,6-dihydro-2H-pyridine-1-carboxylate 3 (0.7 g, 2.0 mmol) and 3-bromopiperidine-2,6-dione 4 (761 mg, 4.0 mmol) in acetonitrile (7 mL) was added sodium bicarbonate (832 mg, 9.9 mmol) and tetrabutylammonium iodide (73.19 mg, 198.16 mol) at room temperature and stirred at 90° C. for 12 hours. Upon completion of the reaction, the reaction mixture was concentrated under reduced pressure to afford the crude product. The crude product was purified by column chromatography (Davisil silica; using 42% ethyl acetate in petroleum ether as an eluent) to afford tert-butyl 4-[2-bromo-4-[(2,6-dioxo-3-piperidyl) amino]phenyl]-3,6-dihydro-2H-pyridine-1-carboxylate 5 (0.6 g, 64% yield) as a pale-green solid. LCMS (ES-): m z 462.14 [M-H]-.

Step-3: A solution of tert-butyl 4-[2-bromo-4-[(2,6-dioxo-3-piperidyl)amino]phenyl]-3,6-dihydro-2H-pyridine-1-carboxylate 5 (0.2 g, 431 mol) in ethanol (1 mL), tetrahydrofuran (2 mL) and ethyl acetate (2 mL) was degassed with N2 for 10 minutes. After, PtO2 (19.2 L, 861 mol) was added and the reaction mixture was stirred under H2 (50 psi) in a 250 mL Parr-shaker vessel for 5 hours. Upon completion of the reaction, the reaction mixture was filtered through a celite bed and washed with ethyl acetate (20 mL) and tetrahydrofuran (20 mL). The filtrate was concentrated under reduced pressure to obtain tert-butyl 4-[2-bromo-4-[(2,6-dioxo-3-piperidyl) amino]phenyl]piperidine-1-carboxylate 6 (0.2 g, 61% yield) as a colorless solid.

LCMS (ES-): m z 464.13 [M-H]-.

Step-4: To a stirred solution of tert-butyl 4-[2-bromo-4-[(2,6-dioxo-3-piperidyl)amino]phenyl]piperidine-1-carboxylate 6 (0.2 g, 429 mol) in isopropyl alcohol (4 mL) were added triethylamine (179 pL, 1.3 mmol) and DABSO (103 mg, 429 mol) at room temperature. The reaction mixture was degassed with argon gas for 10 minutes. Next, Pd(amphos)C12 (15.2 mg, 21.4 mol) was added, and the reaction mixture was stirred at 80° C. for 16 hours. Upon completion of the reaction, the reaction mixture was concentrated under reduced pressure to afford the crude product. The residue obtained was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[4-[(2,6-dioxo-3-piperidyl) amino]-2-fluorosulfonyl-phenyl]piperidine-1-carboxylate 7 (15 mg, 6% yield) as a pale-yellow solid. LCMS (ES-): m z 468.40 [M-H]-.

Step-5: To a stirred solution of tert-butyl 4-[4-[(2,6-dioxo-3-piperidyl) amino]-2-fluorosulfonyl-phenyl]piperidine-1-carboxylate 7 (15 mg, 32.2 mol) in dichloromethane (2 mL) was added trifluoroacetic acid (30.0 L, 386 mol) at 0° C. and stirred at room temperature for 2 hours. Upon completion of the reaction, the reaction mixture was concentrated under reduced pressure to afford the crude product. The residue obtained was purified by preparative HPLC [Column: X-SELECT C18, 19×150 mm, 5 micron, Mobile phase A: 0.1% trifluoroacetic acid in water, Mobile phase B: acetonitrile, Wavelength: 215 nm]and the fractions containing the compound were combined and lyophilized to afford the desired 5 product 5-[(2,6-dioxo-3-piperidyl)amino]-2-(4-piperidyl) benzenesulfonyl fluoride (A-41) (5.3 mg, 34% yield) as an off-white solid. LCMS (ES*): m z 370.31 [M+H]+. ¹H NMR (400 MHz, DMSO-d6): 10.38 (s, 1H), 7.36-7.32 (m, 2H), 7.16-7.14 (m, 1H), 6.73 (d, J=8 Hz, 1H), 4.51 (q, J=8 Hz, 1H), 3.39-3.32 (m, 3H), 3.05 (bs, 2H), 2.74-2.70 (m, 1H), 2.62 (d, J=4.4 Hz, 1H), 2.08 (d, J=4.8 Hz, 2H), 2.00-1.99 (m, 1H), 1.93-1.85 (m, 4H).

Synthesis A42: Synthesis of 5-((2,6-dioxopiperidin-3-yl)oxy)-2-(piperidin-4-yl)phenyl sulfurofluoridate (A-42)

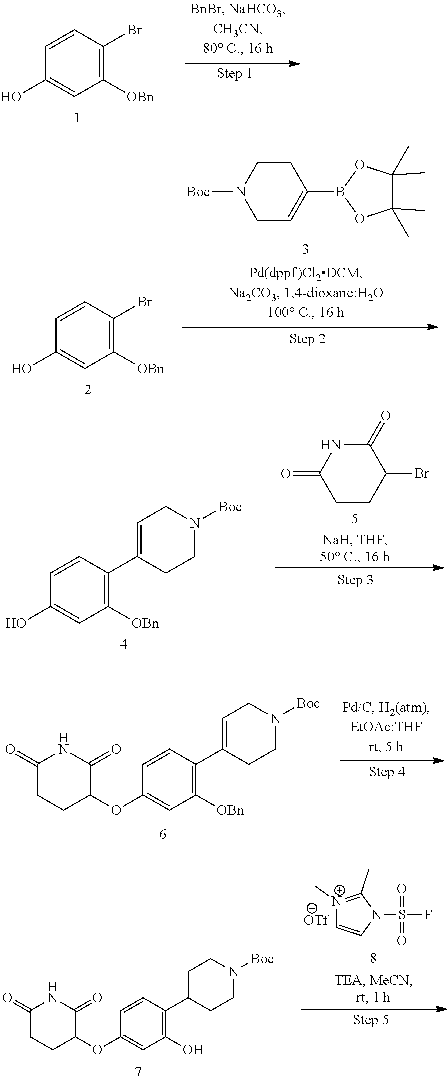

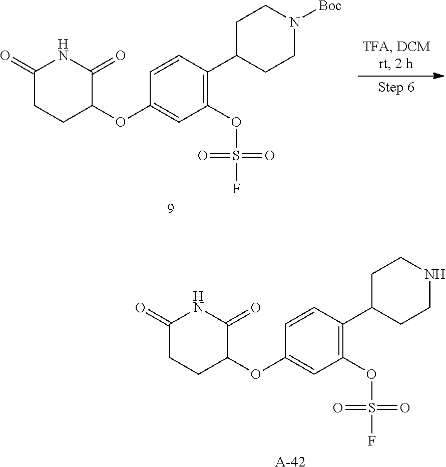 [SMILES: CC(C)(C)OC(=O)N1CCC(c2ccc(OC3CCC(=O)NC3=O)cc2OS(=O)(=O)F)CC1 ; O=C1CCC(Oc2ccc(C3CCNCC3)c(OS(=O)(=O)F)c2)C(=O)N1 ; "A-42"]

Step-1:

To a stirred solution of 4-bromobenzene-1, 3-diol 1 (2 g, 10.6 mmol) in acetonitrile (20 5 mL) were added sodium bicarbonate (1.78 g, 21.16 mmol), bromomethylbenzene (1.26 mL, 10.6 mmol) consecutively and stirred at 80° C. for 16 hours. Upon completion of the reaction, the reaction mixture was filtered and concentrated under reduced pressure to afford the crude product, which was purified by column chromatography (60-100 mesh silica gel, 9% ethyl acetate in petroleum ether as eluent) to afford 3-benzyloxy-4-bromo-phenol 2 (1.2 g, 36% 10 yield) as a colorless solid. LCMS (ES): m z 277.00 [M-H]-.

Step-2: To a stirred solution of 3-benzyloxy-4-bromo-phenol 2 (0.9 g, 3.2 mmol) and tert-butyl 4-(4,4,5,5-tetramethyl-1,3,2-dioxaborolan-2-yl)-3,6-dihydro-2H-pyridine-1-carboxylate 3 (1.2 g, 3.9 mmol) in water (2 mL), 1,4-dioxane (20 mL) was added sodium carbonate (1.03 g, 9.7 15 mmol) at room temperature. The reaction mixture was degassed with argon gas for 10 minutes, after which, [1,1′-bis(diphenylphosphino)ferrocene]dichloropalladium(II) complex (263 mg, 322 mol) was added and the reaction mixture was stirred at 100° C. for 16 hours. Upon completion, the reaction mixture was filtered through celite bed and washed with ethyl acetate (2×10 mL). The filtrate was concentrated under reduced pressure to afford the crude product. 20 The crude product was purified by column chromatography (Davisil silica; using 19% ethyl acetate in petroleum ether as an eluent) to afford tert-butyl 4-(2-benzyloxy-4-hydroxy-phenyl)-3,6-dihydro-2H-pyridine-1-carboxylate 4 (1 g, 78% yield) as a pale-yellow solid. LCMS (ES-): m/z 380.23 [M-H]-.

Step-3: To a stirred solution of tert-butyl 4-(2-benzyloxy-4-hydroxy-phenyl)-3,6-dihydro-2H-pyridine-1-carboxylate 4 (0.5 g, 1.3 mmol) in tetrahydrofuran (10 mL) was added sodium hydride (60% suspension in mineral oil) (79 mg, 3.3 mmol), 3-bromopiperidine-2,6-dione 5 (504 mg, 2.6 mmol) consecutively at 0° C. and stirred at 50° C. for 16 hours. Upon completion of the reaction, the reaction mixture was quenched with saturated ammonium chloride (20 mL) and extracted with ethyl acetate (2×50 mL). The combined organic layer was dried over sodium sulfate and concentrated under reduced pressure to afford the crude product. The crude product was purified by column chromatography (60-120 silica gel, using 49% ethyl acetate and petroleum ether as an eluent) to afford tert-butyl 4-[2-benzyloxy-4-[(2,6-dioxo-3-piperidyl)oxy]phenyl]-3,6-dihydro-2H-pyridine-1-carboxylate 6 (0.25 g, 37% yield) as a colorless solid. LCMS (ES-): m z 491.33 [M-H]-.

Step-4: To a solution of tert-butyl 4-[2-benzyloxy-4-[(2,6-dioxo-3-piperidyl)oxy]phenyl]-3,6-dihydro-2H-pyridine-1-carboxylate 6 (0.25 g, 508 mol) in ethyl acetate (3 mL) and tetrahydrofuran (3 mL) was degassed with N2 for 10 minutes. After, 10% Pd/C (270.07 mg, 2.54 mmol) was added, and the reaction mixture was stirred under H2 (60 psi) at room temperature for 5 hours. Upon completion of the reaction, the reaction mixture was filtered through celite bed and washed with ethyl acetate (20 mL) and tetrahydrofuran (20 mL). The filtrate was concentrated under reduced pressure to afford the crude product tert-butyl 4-[4-[(2,6-dioxo-3-piperidyl) oxy]-2-hydroxy-phenyl]piperidine-1-carboxylate 7 (0.2 g, 62% yield) as a colorless solid. LCMS (ES-): m z 403.28 [M-H]-.

Step-5: To a stirred solution of tert-butyl 4-[4-[(2,6-dioxo-3-piperidyl)oxy]-2-hydroxy-phenyl]piperidine-1-carboxylate 7 (0.1 g, 247 mol) in acetonitrile (2 mL) were added triethylamine (52 L, 371 mol), 1-(fluorosulfonyl)-2,3-dimethyl-1H-imidazol-3-ium trifluoromethanesulfonate 8 (106 mg, 321 mol) and stirred at room temperature for 1 hour.

Upon completion of the reaction, the reaction mixture was concentrated under reduced pressure to afford the crude product. The crude product obtained was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[4-[(2,6-dioxo-3-piperidyl)oxy]-2-fluorosulfonyloxy-phenyl]piperidine-1-carboxylate 9 (65 mg, 53% yield) as an off-white solid. LCMS (ES-): m z 485.38 [M-H]-.

Step-6: To a stirred solution of tert-butyl 4-[4-[(2,6-dioxo-3-piperidyl)oxy]-2-fluorosulfonyloxy-phenyl]piperidine-1-carboxylate 9 (30 mg, 62 mol) in dichloromethane (1.5 mL) was added trifluoroacetic acid (57 pL, 740 mol) at 0° C. and stirred at room temperature for 2 hours. Upon completion, the reaction mixture was concentrated under reduced pressure to afford the crude product. The residue obtained was triturated with diethyl ether (10 mL) to afford 3-[3-fluorosulfonyloxy-4-(4-piperidyl) phenyl]-2,6-dioxo-piperidine (A-42) (27.4 mg, 88% yield) as an off-white solid. LCMS (ES*): m z 387.30 [M+H]+. ¹H NMR (400 MHz, DMSO-d6): δ 10.99 (s, 1H), 7.37 (d, J=8.8 Hz, 1H), 7.26-7.19 (m, 2H), 5.33 (m, 1H), 3.39-3.32 (m, 2H), 3.09-3.08 (m, 3H), 2.70-2.60 (m, 2H), 2.25-2.15 (m, 2H), 1.90-1.80 5 (m, 5H).

Synthesis A43: Synthesis of 5-(2,4-dioxotetrahydropyrimidin-1(2H)-yl)-2-(piperidin-4-yl)benzenesulfonyl fluoride (A-45)

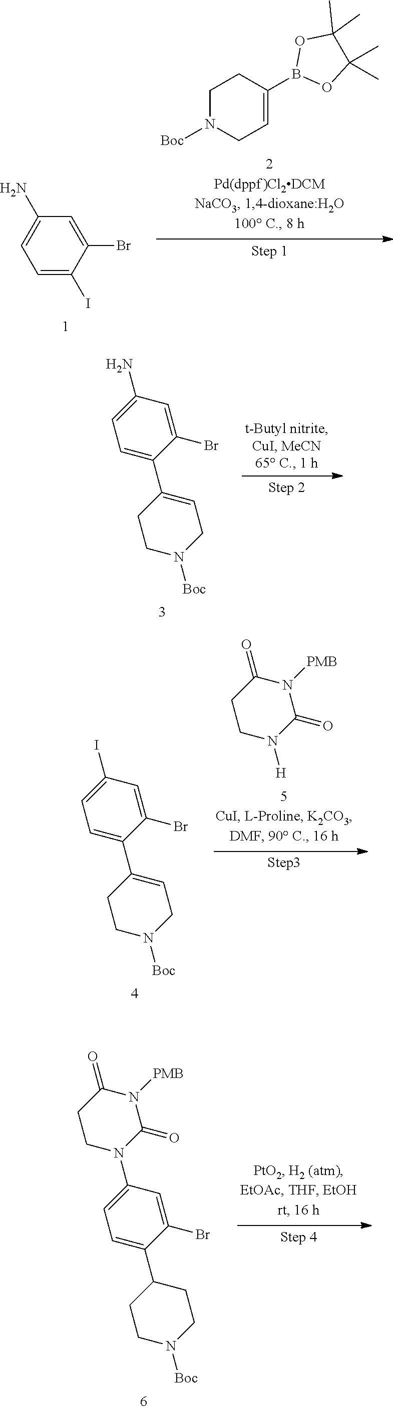

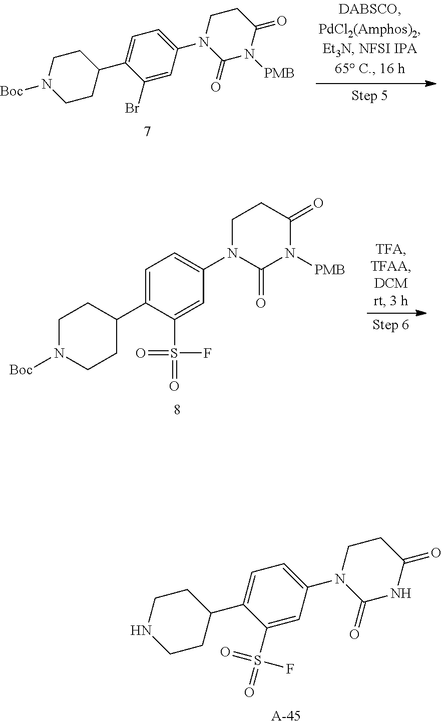

To a stirred solution of 3-bromo-4-iodo-aniline 1 (5.00 g, 16.8 mmol) and tert-butyl 4-(4,4,5,5-tetramethyl-1,3,2-dioxaborolan-2-yl)-3,6-dihydro-2H-pyridine-1-carboxylate 2 (4.7 5 g, 15.1 mmol) in 1,4-dioxane (15 mL), water (5 mL) was added tripotassium phosphate (8.72 g, 50.4 mmol) and purged with N2 for 10 minutes. Next, Pd(dppf)C12 (1.23 g, 1.7 mmol) was added and the reaction mixture was stirred at 100° C. for 8 h. Upon completion, the reaction mixture was diluted with water (100 mL) and extracted with ethyl acetate (3×100 mL). The combined organic layer was dried over anhydrous sodium sulfate and concentrated under 10 reduced pressure to afford the crude product, which was purified by column chromatography (Davisil silica; using 8-10% ethyl acetate in pet-ether as an eluent) to afford tert-butyl 4-(4-amino-2-bromo-phenyl)-3,6-dihydro-2H-pyridine-1-carboxylate 3 (4.15 g, 56% yield) as pale-brown solid. LCMS (ES-): m/z 297.08 [M-56]-.

Step-2: To a stirred solution of tert-butyl 4-(4-amino-2-bromo-phenyl)-3,6-dihydro-2H-pyridine-1-carboxylate 3 (5.0 g, 14.2 mmol), CuI (3.50 g, 18.4 mmol) in acetonitrile (50 mL) was added t-butyl nitrite (3.4 mL, 28.3 mmol) at room temperature and stirred at 65° C. for 1 hour. Upon completion of the reaction, the reaction mixture was filtered through celite bed and washed with ethyl acetate (3×50 mL). The filtrate was concentrated under reduced pressure 20 to afford the crude product, which was purified by column chromatography (Davisil silica; using 15% ethyl acetate in pet-ether as an eluent) to afford tert-butyl 4-(2-bromo-4-iodo-phenyl)-3,6-dihydro-2H-pyridine-1-carboxylate 4 (2.5 g, 25% yield) as an off-white solid.

LCMS (ES): m/z 408.07 [M-56]*.

Step-3: To a solution of tert-butyl 4-(2-bromo-4-iodo-phenyl)-3,6-dihydro-2H-pyridine-1-carboxylate 4 (2.50 g, 5.4 mmol) in 1,4-dioxane (25 mL) were added 3-[(4-methoxyphenyl) methyl]hexahydropyrimidine-2,4-dione 5 (1.89 g, 8.1 mmol), K3PO4 (3.43 g, 16.2 mmol), rac-(1R,2R)-cyclohexane-1,2-diamine (308 mg, 2.7 mmol) and purged for 5 minutes. Next, CuI (513 mg, 2.69 mmol) was added, and the reaction mixture was stirred at 90° C. for 6 hours. 30 Upon completion of the reaction, the reaction mixture was diluted with water (30 mL) and extracted with ethyl acetate (3×50 mL). The combined organic layer dried over anhydrous sodium sulfate and concentrated under reduced pressure to afford crude product, which was purified by column chromatography (Davisil silica; using₃₅-4₀% ethyl acetate in pet-ether as an eluent) to afford tert-butyl 4-[2-bromo-4-[3-[(4-methoxyphenyl)methyl]-2,4-dioxo-hexahydropyrimidin-1-yl]phenyl]-3,6-dihydro-2H-pyridine-1-carboxylate 6 (1.5 g, 34% yield) as an viscous oil. LCMS (ES): m/z 470.32 [M-100]*.

Step-4: To a stirred solution of tert-butyl 4-[2-bromo-4-[3-[(4-methoxyphenyl) methyl]-2,4-dioxo-hexahydropyrimidin-1-yl]phenyl]-3,6-dihydro-2H-pyridine-I-carboxylate 6 (1.2 g, 10 2.10 mmol) in ethyl acetate (100 mL), tetrahydrofuran (100 mL) and ethanol (20 mL) was added platinum (IV) oxide (1.19 g, 5.26 mmol) and stirred under H2 (50 psi) in par shaker at room temperature 16 hours. Upon completion of the reaction, the reaction mixture was filtered over a celite bed and washed with ethyl acetate (3×50 mL). The filtrate was evaporated under reduced pressure to obtain tert-butyl 4-[2-bromo-4-[3-[(4-methoxyphenyl)methyl]-2,4-dioxo-hexahydropyrimidin-1-yl]phenyl]piperidine-1-carboxylate 7 (0.80 g, 44% yield) as pale-yellow liquid. LCMS (ES): m z 516.36 [M-56]*.

Step-5: To a solution of tert-butyl 4-[2-bromo-4-[3-[(4-methoxyphenyl) methyl]-2,4-dioxo-hexahydropyrimidin-1-yl]phenyl]piperidine-1-carboxylate 7 (0.750 g, 1.3 mmol) in isopropyl alcohol (4.6 mL) was added DABSO (472 mg, 2.0 mmol) and triethylamine (548 pL, 3.93 mmol) at room temperature. The reaction mixture was purged with N2 for 10 minutes, after which PdCl2(amphos)2 (93 mg, 131 mol) was added and the reaction mixture was stirred at 65° C. for 16 hours. After, the reaction mixture was cooled to room temperature and NFSI (127 mg, 655 mol) was added, and the reaction mixture was stirred at room temperature for 2 hours.

Upon completion of the reaction, the reaction mixture was quenched with water (10 mL) and extracted with ethyl acetate (3×20 mL). The combined organic layer was dried over anhydrous sodium sulfate and concentrated under reduced pressure to afford the crude product. The crude product was purified by preparative HPLC [Column: X-BRIDGE C18, 19×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product tert-butyl 4-[2-fluorosulfonyl-4-[3-[(4-methoxyphenyl)methyl]-2,4-dioxo-hexahydropyrimidin-1-yl]phenyl]piperidine-1-carboxylate 8 (0.095 g, 12% yield) as an off-white solid. LCMS (ES): m/z 476.34 [M-100]*.

Step-6: To a stirred solution of tert-butyl 4-[2-fluorosulfonyl-4-[3-[(4-methoxyphenyl)methyl]-2,4-dioxo-hexahydropyrimidin-1-yl]phenyl]piperidine-1-carboxylate 8 (100 mg, 174 mol) in dichloromethane (2 mL) was added trifluoroacetic acid (133 L, 1.7 mmol) and triflic acid (458 L, 5.2 mmol) at 0° C. and stirred at room temperature for 3 hours.

Upon completion of the reaction, the reaction mixture was concentrated under reduced pressure to afford the crude product, which was triturated with diethyl ether (2×10 mL) and lyophilized to afford 5-(2,4-dioxohexahydropyrimidin-1-yl)-2-(4-piperidyl)benzenesulfonyl fluoride (A-45) (36 mg, 40% yield) as an off-white solid. LCMS (ES*): m/z 356.32 [M+H]+. ¹H NMR (400 MHz, DMSO-d6) δ: 10.58 (s, 1H), 8.10 (d, J=2.00 Hz, 1H), 7.90-7.91 (m, 1H), 7.70 (d, J=8.80 Hz, 2H), 3.89 (t, J=6.80 Hz, 2H), 3.41-3.45 (m, 3H), 3.10-3.12 (m, 2H), 2.72-2.74 (m, 2H), 1.91-1.92 (m, 4H).

Synthesis A44: Synthesis of 4-((2,4-dioxo-3-azabicyclo[3.1.1]heptan-1-yl)amino)phenyl 15 sulfurofluoridate (A-46)

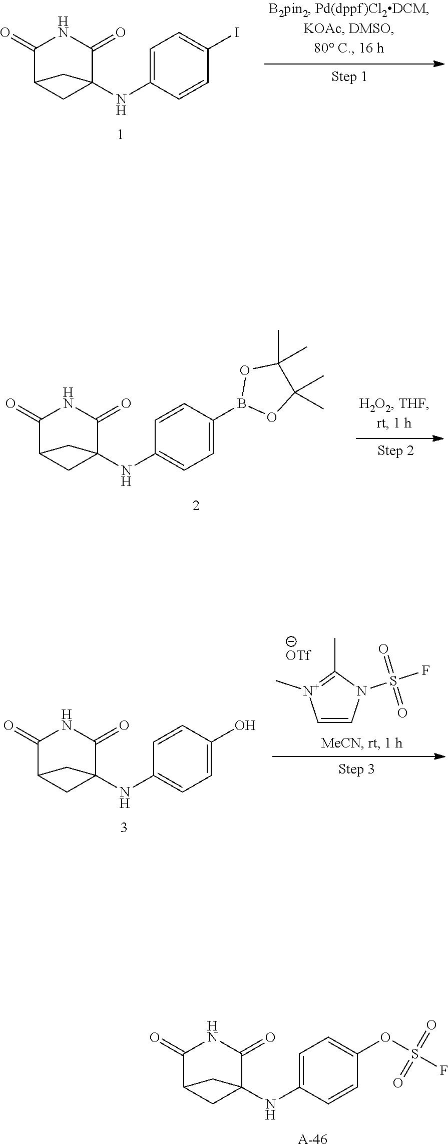

Step-1 To a stirred solution of 1-(4-iodoanilino)-3-azabicyclo[3.1.1]heptane-2,4-dione 1 (0.5 g, 1.5 mmol), 4,4,4′,4′,5,5,5′,5′-Octamethyl-2,2′-bi-1,3,2-dioxaborolane (408 mg, 1.6 mmol) in dimethylsulfoxide (3.89 mL) was added potassium acetate (430 mg, 4.4 mmol) and degassed with N2 for 15 minutes. After, Pd(dppf)C12 DCM (59.7 mg, 73.1 mol) was added, and the reaction mixture was stirred at 80° C. for 16 hours. Upon completion of reaction, the reaction mixture was diluted with water (10 mL) and extracted with ethyl acetate (2×25 mL). The combined organic layer was dried over sodium sulfate, filtered, and concentrated under reduced pressure to afford the crude product. The crude product was purified in flash chromatography (Davisil silica; using 40% ethyl acetate in petroleum ether as an eluent) to afford 1-[4-(4,4,5,5-tetramethyl-1,3,2-dioxaborolan-2-yl)anilino]-3-azabicyclo[3.1.1]heptane-2,4-dione 2 (0.4 g, 41% yield) as yellow oil. LCMS (ES-): m z 341.2 [M-H]-.

Step-2: To a stirred solution of 1-[4-(4,4,5,5-tetramethyl-1,3,2-dioxaborolan-2-yl)anilino]-3-azabicyclo[3.1.1]heptane-2,4-dione 2 (200 mg, 585 mol) in tetrahydrofuran (2 mL) was added H₂O₂ solution (³0% solution in water) (53.7 pL, 1.8 mmol) at 0° C. and stirred at room temperature for 1 hour. Upon completion of reaction, the reaction mixture was quenched with saturated sodium bisulfite solution(2 mL) and extracted with ethyl acetate (2×10 mL). The combined organic layer was concentrated under reduced pressure to afford the crude product 1-(4-hydroxyanilino)-3-azabicyclo[3.1.1]heptane-2,4-dione 3 (0.14 g, 20% yield). LCMS (ES*): m/z 233.14 [M+H]*.

Step-3: To a stirred solution of 1-(4-hydroxyanilino)-3-azabicyclo[3.1.1]heptane-2,4-dione 3 (70 mg, 301 mol) in acetonitrile (2.00 mL) were added triethylamine (67 L, 482 mol,) and 1-(fluorosulfonyl)-2,3-dimethyl-1H-imidazol-3-iumtrifluoromethanesulfonate (129 mg, 393 mol) and stirred at room temperature for 1 hour. Upon completion of the reaction, the reaction mixture was evaporated under reduced pressure to afford the crude product. The crude product was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 1-(4-fluorosulfonyloxyanilino)-2,4-dioxo-3-azabicyclo [3.1.1]heptane (A-46) (8.2 mg, 9% yield) as an off-white solid. ¹H NMR (400 MHz, DMSO-d6) δ: 10.78 (s, 1H), 7.22 (d, J 9.2 Hz, 2H), 6.77 (s, 1H), 6.53 (d, J 9.2 Hz, 2H), 2.97 (t, J 5.8 Hz, 1H), 2.81-2.79 (m, 2H), 2.52-2.50 (m, 2H). LCMS (ES*): m/z 315.30 [M+H]+.

Synthesis A45: Synthesis of 3-((2,6-dioxopiperidin-3-yl)carbamoyl)phenyl sulfurofluoridate (A-47)

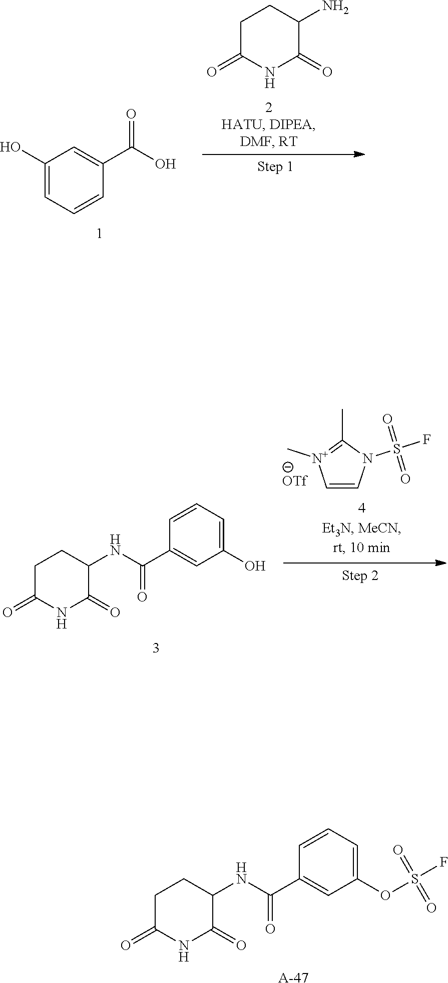 [SMILES: NC1CCC(=O)NC1=O ; O=C(O)c1cccc(O)c1 ; Cc1n(S(=O)(=O)F)cc[n+]1C ; O=C1CCC(NC(=O)c2cccc(O)c2)C(=O)N1 ; O=C1CCC(NC(=O)c2cccc(OS(=O)(=O)F)c2)C(=O)N1 ; "A-47"]

5 Step-1: To a stirred solution of 3-aminopiperidine-2,6-dione 2 (0.5 g, 3.9 mmol) and 3-hydroxybenzoic acid 1 (377 mg, 2.7 mmol) in dimethylformamide (5 mL) were added N,N-diisopropylethylamine (2.0 mL, 11.7 mmol), HATU (2.2 g, 5.9 mmol) and stirred at room temperature for 1 hour. Upon completion of the reaction, the reaction mixture was poured into 10 ice-chilled water (20 mL) to afford a white precipitate. The precipitate was filtered and dried with sodium sulfate to afford N-(2,6-dioxo-3-piperidyl)-3-hydroxy-benzamide 3 (0.4 g, 26% yield) as a brown solid. LCMS (ES-): m z 247.11 [M-H]-.

Step-2: To a stirred solution of N-(2,6-dioxo-3-piperidyl)-3-hydroxy-benzamide 3 (0.150 g, 604 mol) in acetonitrile (3 mL) were added triethylamine (252.67 L, 1.8 mmol), 1-(fluorosulfonyl)-2,3-dimethyl-1H-imidazol-3-ium trifluoromethanesulfonate 4 (199 mg, 604 mol) at 0° C. and stirred at room temperature for 10 minutes. Upon completion of the reaction, the reaction mixture was diluted with water (10 mL) and extracted with ethyl acetate (2×20 mL). The combined organic layer washed with saturated sodium chloride solution (10 mL), 20 dried over anhydrous sodium sulfate and concentrated under reduced pressure to afford the crude product. The crude product was purified by preparative HPLC [Column: X-SELECT C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-[(3-fluorosulfonyloxybenzoyl)amino]-2,6-dioxo-piperidine (A-47) (16 mg, 8% yield) as an off-white solid. LCMS (ES-): m z 329.18 [M-H]-.¹H NMR (400 MHz, DMSO-d6) δ: 10.91 (s, 1H), 9.02 (d, J=8.00 Hz, 1H), 8.03 (d, J=1.20 Hz, 2H), 7.85 (d, J=2.40 Hz, 2H), 7.75 (t, J=16.40 Hz, 1H), 2.86-2.77 (m, 1H), 2.58-2.57 (m, 1H), 2.19-2.11 (m, 1H), 2.09-2.02 (m, 1H).

Synthesis A46: Synthesis of 7-(2,6-dioxopiperidin-3-yl)naphthalen-2-ylsulfurofluoridate (A-48)

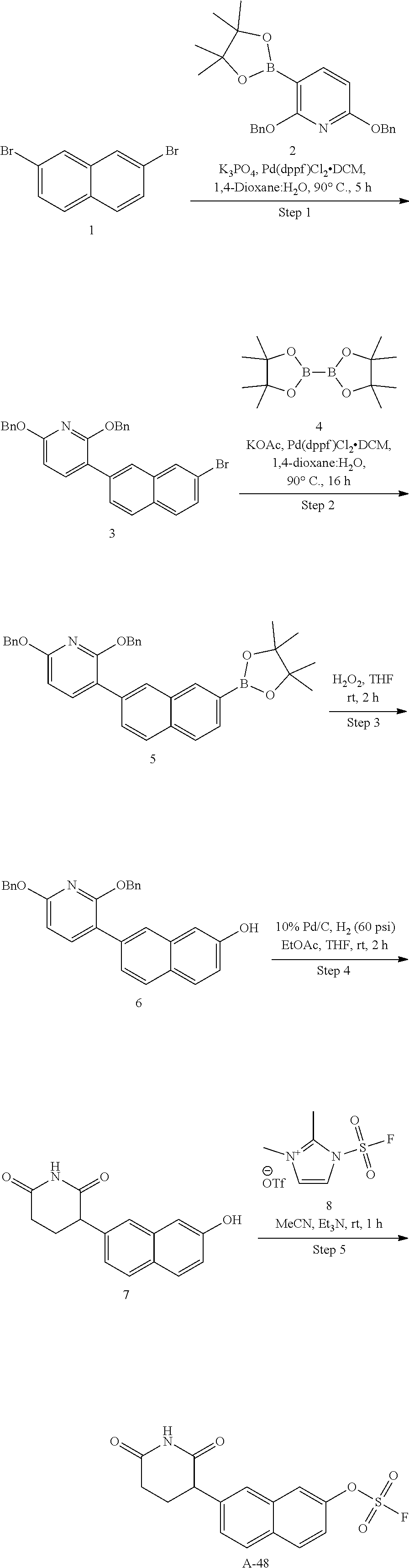

Step-1: To a stirred solution of 2,7-dibromonaphthalene 1 (3 g, 10.5 mmol), 2,6-dibenzyloxy-3-(4,4,5,5-tetramethyl-1,3,2-dioxaborolan-2-yl)pyridine 2 (3.1 g, 7.3 mmol) in 1,4-dioxane (30 mL) and water (6 mL) was added potassium phosphate (4.5 g, 21.0 mmol) at room temperature. 5 The reaction mixture was degassed with nitrogen for 10 minutes before adding Pd(dppf)Cl2 DCM complex (857 mg, 1.1 mmol) and stirred at 90° C. for 5 hours. Upon completion of the reaction, the reaction mixture was filtered over celite bed and washed with ethyl acetate (100 mL). The filtrate was evaporated under reduced pressure to obtain crude product. Which was purified by column chromatography (using Davisil silica and 5% ethyl 10 acetate in petroleum ether as eluent) to afford 2,6-dibenzyloxy-3-(7-bromo-2-naphthyl) pyridine 3 (2.2 g, 30% yield). LCMS (ES*): 496.47 [M+H]+.

Step-2: To a stirred solution of 2,6-dibenzyloxy-3-(7-bromo-2-naphthyl)pyridine 3 (750 mg, 1.51 mmol), 4,4,5,5-tetramethyl-2-(4,4,5,5-tetramethyl-1,3,2-dioxaborolan-2-yl)-1,3,2-dioxaborolane 4 (767.35 mg, 3.02 mmol) in 1,4-dioxane (10 mL) was added potassium acetate (962 mg, 9.8 mmol) at room temperature. The reaction mixture was degassed with N2 for 10 minutes before adding Pd(dppf)C12 DCM complex (99 mg, 121 mol) and stirred at 90° C. for 16 hours. Upon completion of the reaction, the reaction mixture was filtered over celite bed and washed with ethyl acetate (100 mL). The filtrate was evaporated under reduced pressure to obtain crude product, which was purified by column chromatography (using Davisil silica, 5% ethyl acetate in pet-ether as an eluent) to afford 2,6-dibenzyloxy-3-[7-(4,4,5,5-tetramethyl-1,3,2-dioxaborolan-2-yl)-2-naphthyl]pyridine 5 (500 mg, 32% yield). LCMS (ES+): 544.63 [M+H]*.

Step-3: To a stirred solution of 2,6-dibenzyloxy-3-[7-(4,4,5,5-tetramethyl-1,3,2-dioxaborolan-2-yl)-2-naphthyl]pyridine 5 (500 mg, 920 mol) in tetrahydrofuran (6 mL) was added hydrogen peroxide (2.22 g, 65.27 mmol, 2 mL) at 0° C. and stirred at room temperature for 2 hours. Upon completion of the reaction, the reaction mixture was quenched with sodium sulfite solution (20 mL) and extracted with ethyl acetate (3×10 mL). The combined organic layer was dried over anhydrous sodium sulfate and evaporated under reduced pressure to obtain 7-(2,6-dibenzyloxy-3-pyridyl)naphthalen-2-ol 6 (400 mg, 53% yield). LCMS (ES+): 434.53 [M+H]*.

Step-4: To a stirred solution of 7-(2,6-dibenzyloxy-3-pyridyl)naphthalen-2-ol 6 (500 mg, 1.2 mmol) in ethyl acetate (10 mL), tetrahydrofuran (10 mL) was added 10% palladium on carbon (500 mg, 4.70 mmol) at room temperature. The reaction mixture was stirred under H2 atmosphere (40 psi, balloon pressure) at room temperature for 2 hours. Upon completion of the reaction, the reaction mixture was filtered over celite bed and concentrated under reduced pressure to obtain 3-(7-hydroxy-2-naphthyl) piperidine-2,6-dione 7 (300 mg, 59% yield) as an 5 oil. LCMS (ES): 256.32 [M+H]*.

Step-5: To a stirred solution of 3-(7-hydroxy-2-naphthyl) piperidine-2,6-dione 7 (300 mg, 1.2 mmol) in acetonitrile (4 mL) were added triethylamine (213 L, 1.5 mmol), 1-(fluorosulfonyl)-2, 3-dimethyl-1H-imidazol-3-iumtrifluoromethanesulfonate 8 (620 mg, 1.9 mmol) and stirred 10 at room temperature for 1 hour. Upon completion of the reaction, reaction mixture was evaporated under reduced pressure to obtain crude product, which was purified by preparative HPLC [Column: X-BRIDGE C18, 19×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 3-(7-fluorosulfonyloxy-2-naphthyl)-2,6-dioxo-piperidine (A-48) (23 mg, 6% yield) as a solid. LCMS (ES-): 336.15 [M-H]-.HNMR (400 MHz, DMSO-d6) 6:10.93 (s, J=2 Hz, 1H), 8.21 (d, J=2 Hz, 1H), 8.15 (d, J=8.8 Hz, 1H), 8.04 (d, J=8.8 Hz, 1H), 7.92 (s, 1H), 7.01-7.68 (dd, 1H), 7.56-7.54 (dd, 1H), 7.01 (bs, 1H), 4.12-4.08 (dd, 1H), 2.77-2.58 (m, 1H), 2.56-2.54 (m, 1H), 2.40-2.32 (m, 1H), 2.39-2.33 (m, 1H), 2.30-2.12 (m, 1H).

Synthesis A47: Synthesis of 3-((2,6-dioxopiperidin-3-yl)carbamoyl)benzenesulfonyl fluoride (A-49)

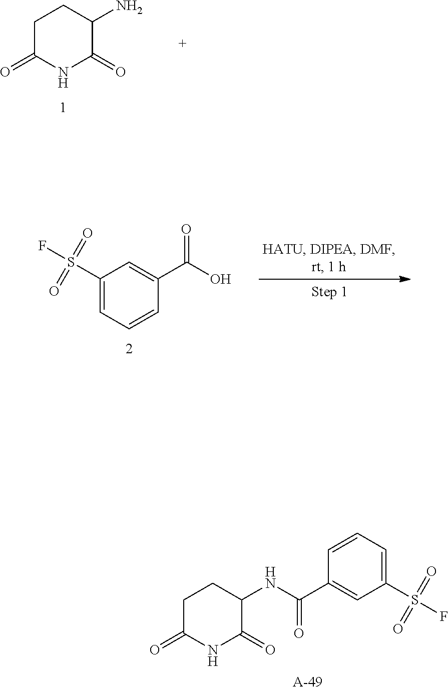

Step-1: To a stirred solution of 3-aminopiperidine-2,6-dione 1 (0.1 g, 780 mol) and 3-fluorosulfonylbenzoic acid 2 (112 mg, 546 mol) in dimethylformamide (2 mL) were added N,N-diisopropylethylamine (410 L, 2.34 mmol), HATU (356 mg, 937 mol) and stirred at room temperature for 1 hour. Upon completion of the reaction, the reaction mixture was concentrated under reduced pressure to obtain crude product, was purified by preparative HPLC [Column: X-SELECT C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-[(2,6-dioxo-3-piperidyl)carbamoyl]benzenesulfonyl fluoride (A-49) (44.3 mg, 18% yield) as an off-white solid. LCMS (ES+): m z 315.30 [M+H]+. H-NMR (400 MHz, DMSO-d6) 6:11.00 (s, 1H), 9.24 5 (d, J=8.00 Hz, 1H), 8.57 (s, 1H), 8.43 (d, J=7.60 Hz, 1H), 8.35 (d, J=8.40 Hz, 1H), 7.95 (t, J=16.00 Hz, 1H), 4.88-4.82 (m, 1H), 2.87-2.78 (m, 1H), 2.59-2.58 (m, 1H), 2.19-2.11 (m, 1H), 2.09-2.06 (m, 1H).

Synthesis A48: Synthesis of 5-(2,6-dioxopiperidin-3-yl)-2-(piperidin-4-yl)benzenesulfonyl 10 fluoride (A-50)

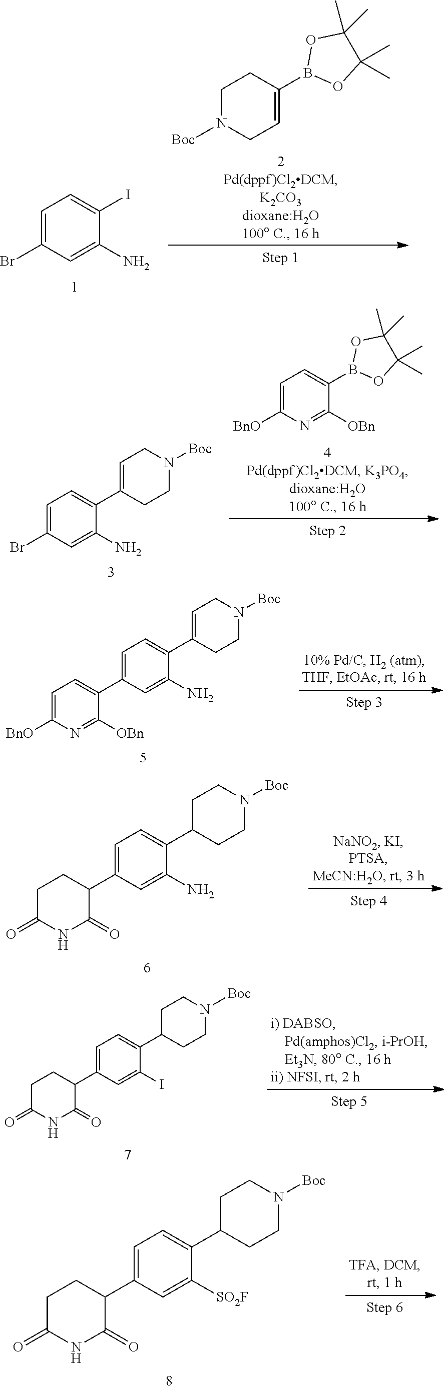

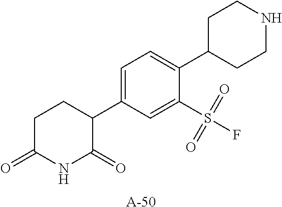

To a solution of 5-bromo-2-iodo-aniline 1 (8 g, 26.9 mmol), tert-butyl 4-(4,4,5,5-tetramethyl-1,3,2-dioxaborolan-2-yl)-3,6-dihydro-2H-pyridine-1-carboxylate 2 (8.30 g, 26.9 5 mmol) in water (10 mL) and 1,4-dioxane (40 mL) was added K2CO3 (11.13 g, 80.6 mmol) at room temperature. The reaction mixture was degassed for 10 minutes with N2 before adding Pd(dppf)Cl2 DCM complex (2.2 g, 2.7 mmol) and stirred at 100° C. for 16 hours. Upon completion of the reaction, the reaction mixture was filtered over celite bed and washed with ethyl acetate (100 mL), the filtrate was concentrated under reduced pressure to afford crude 10 product, which was purified by flash column chromatography (using Davisil silica, 10-20% ethyl acetate in pet-ether as an eluent) to afford tert-butyl 4-(2-amino-4-bromo-phenyl)-3,6-dihydro-2H-pyridine-1-carboxylate 3 (7.5 g, 76% yield) as a light green solid. LCMS (ES): m/z 351.05 [M-H]-.

Step-2: To a solution of tert-butyl 4-(2-amino-4-bromo-phenyl)-3,6-dihydro-2H-pyridine-1-carboxylate 3 (4.5 g, 12.7 mmol) and 2,6-dibenzyloxy-3-(4,4,5,5-tetramethyl-1,3,2-dioxaborolan-2-yl) pyridine 4 (6.4 g, 15.3 mmol) in water (9 mL) and 1,4-dioxane (36 mL) was added K3PO4 (8.11 g, 38.2 mmol) at room temperature. The reaction mixture was degassed with N2 for 10 minutes before adding Pd(dppf)C12 DCM complex (1.04 g, 1.3 mmol) and 20 stirred at 100° C. for 16 hours. Upon completion of the reaction, the reaction mixture was filtered over celite bed and washed with ethyl acetate (100 mL), the filtrate was concentrated under reduced pressure to afford crude product, which was purified by flash column chromatography (using Davisil silica,20% ethyl acetate in pet-ether as an eluent) to afford tert-butyl 4-[2-amino-4-(2,6-dibenzyloxy-3-pyridyl) phenyl]-3,6-dihydro-2H-pyridine-1-carboxylate 5 (6.5 g, 81% yield) as a brown oil. LCMS (ES): m z 564.49 [M+H]*.

Step-3: To a stirred solution of tert-butyl 4-[2-amino-4-(2,6-dibenzyloxy-3-pyridyl)phenyl]-3,6-dihydro-2H-pyridine-1-carboxylate 5 (5.5 g, 9.8 mmol) in ethyl acetate (20 mL) and tetrahydrofuran (20 mL) was added ~10% Pd on carbon (5.2 g, 48.8 mmol) at room temperature. The reaction mixture was stirred under H2 atmosphere (60 psi, Parr shaker) at room temperature for 16 hours. Upon completion of the reaction, the reaction mixture was filtered through celite bed and washed with ethyl acetate (100 mL), the filtrate was concentrated under reduced pressure to afford tert-butyl 4-[2-amino-4-(2,6-dioxo-3-piperidyl) phenyl]piperidine-1-carboxylate 6 (2.9 g, 53% yield) as an off-white solid. LCMS (ES-): m z 386.46 [M-H]-.

Step-4: To a stirred solution of tert-butyl 4-[2-amino-4-(2,6-dioxo-3-piperidyl)phenyl]piperidine-1-carboxylate 6 (1.0 g, 2.58 mmol), PTSA. H20 (1.47 g, 7.74 10 mmol) in acetonitrile (12 mL) at 0° C. were added sodium nitrite (356 mg, 5.2 mmol), KI (1.07 g, 6.5 mmol) in water (2 mL) and stirred at 15° C. for 1.5 hours. Upon completion of the reaction, the reaction mixture was basified (pH 8-10) with 1M sodium bicarbonate solution and extracted with ethyl acetate (3×50 mL). The combined organic layers were dried over sodium sulfate and concentrated under reduced pressure to afford tert-butyl 4-[4-(2,6-dioxo-3-piperidyl)-2-iodo-phenyl]piperidine-1-carboxylate 7 (1.0 g, 38% yield) as a brown solid.

LCMS (ES): m z 443.25 [M-^{t}Bu]*.

Step-5: To a stirred solution of tert-butyl 4-[4-(2,6-dioxo-3-piperidyl)-2-iodo-phenyl]piperidine-1-carboxylate 7 (0.5 g, 1.00 mmol), DABCO(SO2)2 (145 mg, 602 mol) in anhydrous isopropanol (5 mL) was added triethyl amine (420 L, 3.0 mmol) at room temperature. The reaction mixture was degassed with N2 for 10 minutes before adding Pd(amphos)C12 (71 mg, 100 mol) and stirred at 80° C. for 16 hours. Upon being stirred for 16 hours, the reaction mixture was cooled to room temperature and N-fluorobenzenesulfonimide (474.57 mg, 1.50 mmol) was added. The reaction mixture was stirred at room temperature for 2 hours. Upon completion of the reaction, the reaction mixture was concentrated under reduced pressure to obtain the crude, which was dissolved in ethyl acetate (20 mL) and filtered over a celite bed. The filtrate was washed with saturated sodium chloride solution (2×10 mL), dried over sodium sulfate and concentrated under reduced pressure to afford crude product, which was purified by preparative HPLC [Column: X-Bridge C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[4-(2,6-dioxo-3-piperidyl)-2-fluorosulfonyl-phenyl]piperidine-1-carboxylate 8 (65 mg, 14% yield) as a brown solid. LCMS (ES-): m z 453.36 [M-H]-. Step-6: To a stirred solution of tert-butyl 4-[4-(2,6-dioxo-3-piperidyl)-2-fluorosulfonyl-phenyl]piperidine-1-carboxylate 8 (65 mg, 143 mol) in dichloromethane (2 mL) were added trifluoroacetic acid (131.33 L, 1.72 mmol) at 0° C. The reaction mixture was stirred at room 5 temperature for 1 hour. Upon completion of the reaction, the reaction mixture was evaporated under reduced pressure to afford the crude product, which was triturated with diethyl ether (2×5 mL) and dried under reduced pressure to afford desired product, which was lyophilized using acetonitrile:water (1:3) to afford 5-(2,6-dioxo-3-piperidyl)-2-(4-piperidyl) benzene sulfonyl fluoride (A-50) (58 mg, 86% yield, Trifluoroacetic acid) as light brown solid. LCMC 10 (ES*): m z 355.20 [M+H]+. ¹H NMR (400 MHz, DMSO-d6) δ: 10.96 (s, 1H), 8.71 (d, J=1.6 Hz, 1H), 8.40-8.37 (m, 1H), 8.01 (s, 1H), 7.88-7.86 (m, 1H), 7.69 (d, J=8.0 Hz, 1H), 4.17-4.12 (m, 1H), 3.50-3.11 (m, 3H), 3.20-3.01 (m, 2H), 2.70-2.66 (m, 1H), 2.59-2.54 (m, 1H), 2.35-2.30 (m, 1H), 2.08-1.91 (m, 5H).

Synthesis A49: Synthesis of 5-(2,4-dioxotetrahydropyrimidin-1(2H)-yl)-2-(piperazin-1-yl)benzenesulfonyl fluoride (A-51)

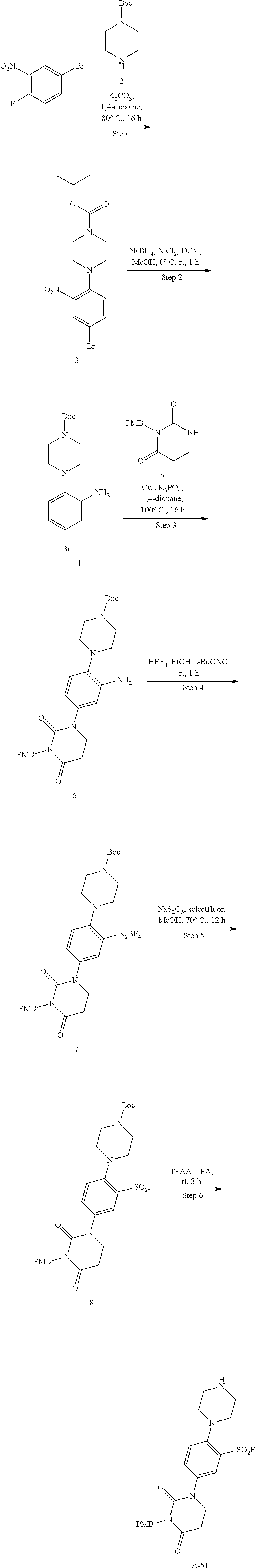

To a stirred solution of 4-bromo-1-fluoro-2-nitro-benzene 1 (8.40 mL, 68.2 mmol) and 5 tert-butyl piperazine-1-carboxylate 2 (10 g, 55 mmol) in dimethylformamide (50 mL) was added sodium K2CO3 (23.6 g, 171 mmol) at room temperature and stirred at 80° C. for 16 hours.

Upon completion, the reaction mixture was diluted with water (150 mL) and extracted with ethyl acetate (3×200 mL). The combined organic layer was washed with saturated sodium chloride solution (100 mL), dried over anhydrous sodium sulfate and concentrated under 10 reduced pressure to afford crude product, which was purified by column chromatography (using Davisil silica, 10% ethyl acetate in petroleum ether as eluent) to afford tert-butyl 4-(4-bromo-2-nitro-phenyl) piperazine-1-carboxylate 3 (12 g, 40% yield). LCMS (ES): m z 286.14[M-100]*.

Step-2: To a stirred solution of tert-butyl 4-(4-bromo-2-nitro-phenyl) piperazine-1-carboxylate 3 (0.75 g, 1.9 mmol) in dichloromethane (7 mL) and methanol (7 mL) were added sodium borohydride (147 mg, 3.9 mmol, 136.80 L) and nickel(II) chloride (25 mg, 194 mol) consecutively at 0° C. and stirred for 1 hour at room temperature. Upon completion of the reaction, the reaction mixture was diluted with water (20 mL) and extracted with dichloromethane (2×20 mL), the combined organic layer was dried with sodium sulfate and concentrated under reduced pressure to afford the crude product tert-butyl 4-(2-amino-4-bromo-phenyl)piperazine-1-carboxylate 4 (0.65 g, 55% yield) as a reddish brown solid. LCMS (ES): m z 300.14 [M-56]*.

Step-3: To a stirred solution of tert-butyl 4-(2-amino-4-bromo-phenyl)piperazine-1-carboxylate 4 (0.65 g, 1.8 mmol) and 3-[(4-methoxyphenyl)methyl]hexahydropyrimidine-2,4-dione 5 (513 mg, 2.2 mmol) in dioxane (13 mL) was added tripotassium phosphate (1.2 g, 5.5 mmol) at room temperature. The reaction mixture was degassed with argon gas for 10 minutes before adding copper iodide (70 mg, 365 mol) and rac-(1R,2R)-cyclohexane-1,2-diamine (42 mg, 365 mol) and stirred at 100° C. for 16 hours. Upon completion of the reaction, the reaction mixture was filtered through celite bed and washed with ethyl acetate (2×20 mL). The filtrate was concentrated under reduced pressure to afford the crude product. The crude product was purified by column chromatography (Davisil silica; using ~₅₂% ethyl acetate in petroleum ether as eluent) to afford tert-butyl 4-[2-amino-4-[3-[(4-methoxyphenyl)methyl]-2,4-dioxo-hexahydropyrimidin-1-yl]phenyl]piperazine-1-carboxylate 6 (0.35 g, 32% yield) as a pale-yellow solid. LCMS (ES): m z 410.52 [M-100]*.

Step-4: To a stirred solution of tert-butyl 4-[2-amino-4-[3-[(4-methoxyphenyl)methyl]-2,4-dioxo-hexahydropyrimidin-1-yl]phenyl]piperazine-1-carboxylate 6 (0.25 g, 490 mol) in ethanol (3 mL) was added trifluoroborane;hydrofluoride (62 pL, 981 mol) and tert-butyl nitrite (116.70 L, 982 mol) at 0° C. and stirred for 1 hour at room temperature. The reaction mixture was evaporated under reduced pressure at 35° C. to afford crude product, which was triturated with diethyl ether (5 mL×2) and dried under reduced pressure to obtain [2-(4-tert-butoxycarbonylpiperazin-1-yl)-5-[3-[(4-methoxyphenyl)methyl]-2,4-dioxo-hexahydropyrimidin-1-yl]phenyl]iminoiminio-trifluoro-boron fluoride 7 (0.23 g, 30% yield) as a reddish-brown solid. LCMS (ES): m z 493.57 [M-115]*. (Note: m/z 493.57 indicates Dess-diazo. HBF4 fragment) Step-5: To a stirred solution of [2-(4-tert-butoxycarbonylpiperazin-1-yl)-5-[3-[(4-methoxyphenyl)methyl]-2,4-dioxo-hexahydropyrimidin-1-yl]phenyl]iminoiminio-trifluoro-boron fluoride 7 (70 mg, 115.1 mol) in anhydrous methanol (2.5 mL) added sodium metabisulfite (66 mg, 345.2 mol) and purged with N2 for 5 minutes before adding Selectfluor (82 mg, 230 mol) and stirred at 70° C. for 12 hours. Upon completion of the reaction, the reaction mixture was filtered through celite bed and washed with methanol (2×5 mL). The filtrate was concentrated under reduced pressure to afford the crude product. The crude product was purified by preparative HPLC [Column: X-Bridge C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[2-fluorosulfonyl-4-[3-[(4-methoxyphenyl)methyl]-2,4-dioxo-hexahydropyrimidin-1-yl]phenyl]piperazine-1-carboxylate 8 (25 mg, 32% yield) as an off-white solid. LCMS (ES*): m z 577.74 [M+H]+.

Step-6: To a stirred solution of tert-butyl 4-[2-fluorosulfonyl-4-[3-[(4-methoxyphenyl)methyl]-2,4-dioxo-hexahydropyrimidin-1-yl]phenyl]piperazine-1-carboxylate 8 (25 mg, 43 mol) in dichloromethane (2 mL) were added trifluoroacetic acid (33 L, 434 mol) and trifluoromethanesulfonic acid (76 L, 867 mol) at 0° C. and was stirred at room temperature for 3 hours. Upon completion of the reaction, the reaction mixture was concentrated under reduced pressure to afford the crude product. The crude product was triturated with diethyl ether (2×5 mL) and lyophilized to afford 5-(2,4-dioxohexahydropyrimidin-1-yl)-2-piperazin-1-yl-benzenesulfonyl fluoride (A-51) (19 mg, 83.21% yield, trifluoromethanesulfonic acid) as a brown solid. LCMS (ES*): m z 357.23 [M+H]+. ¹H NMR (400 MHz, DMSO-d6) 6:10.55 (s, 1H), 8.67 (bs, 1H), 8.03 (d, J=2.40 Hz, 1H), 7.87 (dd, J=2.40 Hz, 1H), 7.75 (d, J=8.40 Hz, 1H), 3.87 (t, J=6.80 Hz, 2H), 3.23 (bs, 4H), 3.14-3.13 (m, 4H), 2.75 (t, J=6.80 Hz, 2H).

Synthesis A50: Synthesis of 3-(2,6-dioxopiperidin-3-yl)-1-methyl-6-(piperidin-4-yl)-1H-indazol-7-yl sulfurofluoridate (A-52)

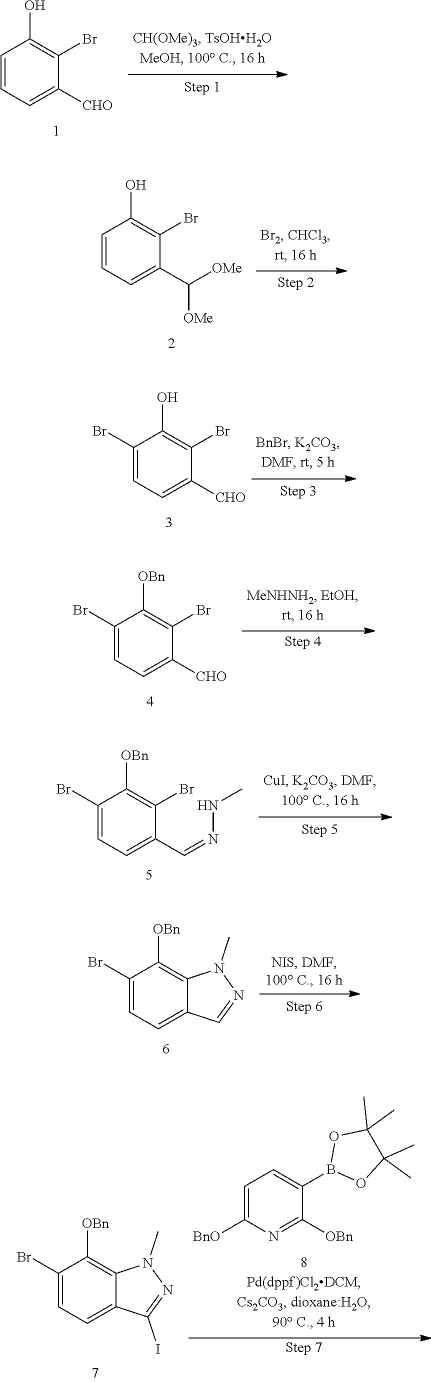

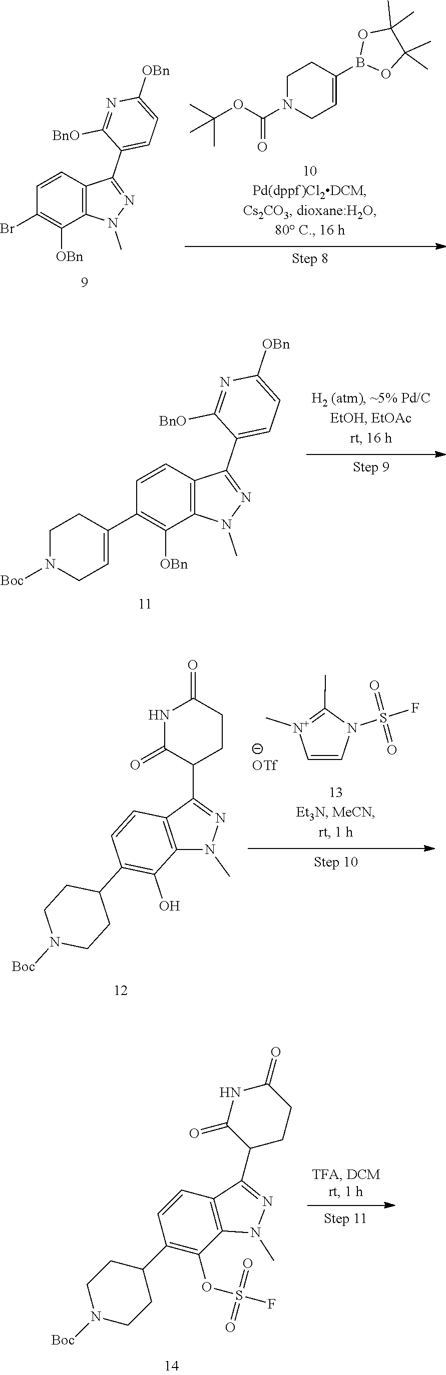

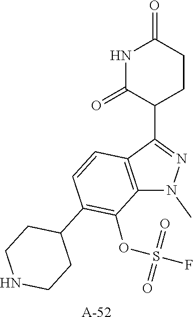

5 Step-1: To a solution of 2-bromo-3-hydroxy-benzaldehyde 1 (6.0 g, 29.9 mmol) and trimethyl orthoformate (9.8 mL, 89.5 mmol) in methanol (80 mL) was added p-toluenesulfonic acid monohydrate (5.7 g, 29.7 mmol) at room temperature. The reaction mixture was stirred at 100° C. for 16 hours. The reaction mixture concentrated under reduced pressure to get the crude product, which was purified by flash chromatography (Davisil silica,using 40% of ethyl acetate in petroleum ether as an eluent) to afford N-[(E)-(3-benzyloxy-2,4-dibromo-phenyl) methyleneamino]methanamine 2 (3.6 g, 84% yield) as an off-white solid. LCMS (ES-): m z 247.01[M-H]-.

Step-2: To a stirred solution of 2-bromo-3-(dimethoxymethyl)phenol 2 (3.5 g, 14.2 mmol) in chloroform (17 mL) was added a solution of bromine (726 L, 14.2 mmol) in chloroform (17 mL) dropwise at 0° C. After being stirred at the room temperature for 16 hours, the reaction was quenched with aqueous saturated sodium thiosulfate solution (30 mL). The organic layer was separated, and the aqueous layer was extracted with ethyl acetate (3×20 mL). The combined organic layers were washed successively with water (100 mL) and saturated sodium chloride solution (100 mL), dried over sodium sulfate, and concentrated in under reduced pressure to obtained 2,4-dibromo-3-hydroxy-benzaldehyde 3 (3.5 g, 85% yield) as a light-yellow solid. LCMS (ES-): m z 276.9 [M-H]-.

Step-3: To a solution of 2,4-dibromo-3-hydroxy-benzaldehyde 3 (3.5 g, 12.5 mmol) and potassium carbonate (5.2 g, 37.5 mmol) in dimethylformamide (25 mL) was added benzyl bromide (1.8 mL, 15.0 mmol) at 0° C. The reaction mixture was stirred at room temperature for 5 hours. The reaction mixture was diluted with water (100 mL) and extracted with ethyl acetate (3×200 mL). The combined organic layers were washed with saturated sodium chloride solution (50 mL), dried over sodium sulfate, filtered and concentrated under reduced pressure to get the crude product, which was triturated with n-pentane (50 mL) to afford 3-benzyloxy-2,4-dibromo-benzaldehyde 4 (3.5 g, 72% yield) as light-yellow solid. ¹H NMR (400 MHz, DMSO-d6) δ: 10.20 (s, 1H), 7.92-7.89 (m, 1H), 7.62-7.57 (m, 3H), 7.47-7.41 (m, 3H), 5.03 (s, 2H).

Step-4: To a solution of 3-benzyloxy-2,4-dibromo-benzaldehyde 4 (3.5 g, 9.5 mmol) in ethanol (39 mL) was added methylhydrazine (872 mg, 18.9 mmol) at 0° C. The reaction mixture stirred at 100° C. for 16 hours. Upon completion of the reaction, the reaction mixture was diluted with water (50 mL) and extracted with ethyl acetate (3×100 mL). The combined organic layers were washed with saturated sodium chloride solution (50 mL), dried over sodium sulfate, filtered and concentrated under reduced pressure to obtained crude product, which was triturated with n-pentane (30 mL) to afford N-[(E)-(3-benzyloxy-2,4-dibromo-phenyl) methyleneamino]methanamine 5 (3.6 g, 84% yield) as a light-yellow solid. LCMS (ES*): m z 397.13 [M+H]+.

Step-5: To a solution of N-[(E)-(3-benzyloxy-2,4-dibromo-phenyl)methyleneamino]methanamine 5 (3.6 g, 9.0 mmol) and K2CO3 (3.75 g, 27.1 mmol) in dimethylformamide (35 mL) was degassed using N2 gas for 5 minutes then CuI (172 mg, 904 mol) was added. The reaction mixture was heated and stirred at 100° C. for 16 hours. The reaction mixture was quenched with water (100 mL) and extracted with ethyl acetate (2×100 mL). The combined organic layer was dried over sodium sulfate and concentrated under reduced pressure to afford crude product, which was purified in flash column chromatography (Davisil silica, using 20% of ethyl acetate in petroleum ether as an eluent) to afford 7-benzyloxy-6-bromo-1-methyl-indazole 6 (1.6 g, 39% yield) as yellow color solid. LCMS (ES-): m z 315.21[M-H]-.

Step-6: To a solution of 7-benzyloxy-6-bromo-1-methyl-indazole 6 (1.6 g, 5.0 mmol) in dimethylformamide (15 mL) were added NIS (2.84 g, 12.6 mmol) at 0° C. The reaction mixture was heated at 100° C. for 16 hours. The reaction mixture was quenched with saturated sodium thiosulfate solution (50 mL) and extracted with ethyl acetate (2×100 mL). The combined organic layer was treated with water (50 mL) and saturated sodium chloride solution (50 mL), dried over sodium sulfate and concentrated under reduced pressure to afford crude product, which was purified in flash column chromatography (Davsil silica gel, 20% of ethyl acetate in petroleum ether as an eluent) to afford 7-benzyloxy-6-bromo-3-iodo-1-methyl-indazole 7 (1.77 g, 50% yield) as a light-yellow solid. LCMS (ES*): m z 445.25 [M+H]+. Step-7: To a solution of 7-benzyloxy-6-bromo-3-iodo-1-methyl-indazole 7 (0.8 g, 1.8 mmol), 2,6-dibenzyloxy-3-(4,4,5,5-tetramethyl-1,3,2-dioxaborolan-2-yl)pyridine 8 (527 mg, 1.3 mmol) in 1,4-dioxane (10 mL), water (2 mL) was added Cs2CO3 (1.76 g, 5.42 mmol) and degassed for 10 minutes using N2. After, Pd(dppf)C12.CH2Cl2 (148 mg, 180.6 mol) was added to the reaction mixture at room temperature and again the reaction was purged for 5 minutes using N2. The reaction mixture was heated and stirred at 90° C. for 4 hours. Upon completion of reaction, the reaction mixture was filtered through celite bed and washed with ethyl acetate (30 mL). The filtrate was concentrated under reduced pressure to afford crude product, which was purified in flash column chromatography (Davisil silica, 20% of ethyl acetate in petroleum ether as eluent) to afford 7-benzyloxy-6-bromo-3-(2,6-dibenzyloxy-3-pyridyl)-1-methyl-indazole 9 (0.443 g, 36% yield) as light-yellow solid. LCMS (ES*): m z 606.43[M+H]+.

Step-8: To a solution of 7-benzyloxy-6-bromo-3-(2,6-dibenzyloxy-3-pyridyl)-1-methyl-indazole 9 (400 mg, 660 mol), tert-butyl 4-(4,4,5,5-tetramethyl-1,3,2-dioxaborolan-2-yl)-3,6-dihydro-2H-pyridine-1-carboxylate 10 (245 mg, 791 mol) in 1,4-dioxane (6 mL), water (1.5 mL) was added sodium carbonate (230.67 mg, 2.18 mmol) and the reaction was degassed for 10 minutes using N2. Next, Pd(dppf)Cl2 DCM (26.93 mg, 32.98 mol) was added to the reaction at room temperature and again the reaction mixture was purged for 5 minutes using N2. The reaction mixture was heated at 80° C. for 16 hours. The reaction mixture was filtered over celite pad and washed with ethyl acetate (30 mL). The filtrate was concentrated under reduced pressure to afford crude product, which was purified in flash column chromatography (Davisil silica, 10% of ethyl acetate in petroleum ether as an eluent) to afford tert-butyl 4-[7-benzyloxy-3-(2,6-dibenzyloxy-3-pyridyl)-1-methyl-indazol-6-yl]-3,6-dihydro-2H-pyridine-1-carboxylate 11 (0.23 g, 39% yield) as light green solid. LCMS (ES*): m z 709.73[M+H]+.

Step-9: A stirred solution of tert-butyl 4-[7-benzyloxy-3-(2,6-dibenzyloxy-3-pyridyl)-1-methyl-indazol-6-yl]-3,6-dihydro-2H-pyridine-1-carboxylate 11 (200 mg, 282.2 mol) in ethyl acetate (2 mL) and tetrahydrofuran (2 mL) was degassed with N2 for 10 minutes and then 5% palladium on carbon wet (240.2 mg, 2.3 mmol) was added into the reaction mixture. The reaction mixture was stirred under H2 atmosphere (Parr shaker, 50 psi) at room temperature for 16 hours. The reaction mixture was filtered over celite pad and washed with ethyl acetate (30 mL), the filtrate was concentrated under reduced pressure to afford crude product tert-butyl 4-[3-(2,6-dioxo-3-piperidyl)-7-hydroxy-1-methyl-indazol-6-yl]piperidine-1-carboxylate 12 (0.140 g, 36% yield) as light green solid. LCMS (ES*): m z 443.40 [M+H]+.

Step-10: To a stirred solution of tert-butyl 4-[3-(2,6-dioxo-3-piperidyl)-7-hydroxy-1-methyl-indazol-6-yl]piperidine-1-carboxylate 12 (0.12 g, 271.18 mol) in acetonitrile (3 mL) were added triethanolamine (43.91 mg, 433.89 mol) and 1-(fluorosulfonyl)-2,3-dimethyl-1H-imidazol-3-ium trifluoromethanesulfonate 13 (116.08 mg, 353.62 mol) at room temperature and continued the reaction for 1 hour. After completion, the solvent was removed under reduced pressure to get crude product, which was purified by preparative HPLC [Column: X-SELECT C18, 19×100 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[3-(2,6-dioxo-3-piperidyl)-7-fluorosulfonyloxy-1-methyl-indazol-6-yl]piperidine-1-carboxylate 14 (19 mg, 13% yield) as an off-white solid. LCMS (ES*): m z 525.29 [M+H]+.

Step-11: To a stirred solution of tert-butyl 4-[3-(2,6-dioxo-3-piperidyl)-7-fluorosulfonyloxy-1-methyl-indazol-6-yl]piperidine-1-carboxylate 14 (0.017 g, 32.41 mol) in dichloromethane (1 mL) was added trifluoroacetic acid (44.34 mg, 388.90 mol, 29.76 L) at 0° C. and the reaction mixture was stirred at room temperature for 1 hour. Upon completion, the reaction mixture was concentrated under reduced pressure to get the crude product, which was triturated with diethyl 10 ether (2×5 mL) and dried at high vacuum, which was lyophilized using CH3CN:H20 (1:3) at −80° C. to afford 3-(2,6-dioxo-3-piperidyl)-7-fluorosulfonyloxy-1-methyl-6-(4-piperidyl)indazole (A-52) (10.3 mg, 58% yield, Trifluoroacetic acid) as an off-white solid.

LCMS (ES+): m z 425.32 [M+H]+. ¹H NMR (400 MHz, DMSO-d6) 6:10.96 (s, 1H), 7.91 (d, J 8.4 Hz, 1H), 7.14 (d, J 8.4 Hz, 1H), 4.46-4.42 (m, 1H), 4.15 (s, 3H), 3.43-3.34 (m, 3H), 3.17-3.09 (m, 2H), 2.77-2.70 (m, 1H), 2.66-2.59 (m, 1H), 2.43-2.38 (m, 1H), 2.20-2.18 (m, 1H), 2.01-1.88 (m, 4H).

Synthesis A51: Synthesis of 1-(2,6-dioxopiperidin-3-yl)-3-methyl-2-oxo-5-(piperidin-4-yl)-2,3-dihydro-1H-benzo[d]imidazol-4-yl sulfurofluoridate (A-53)

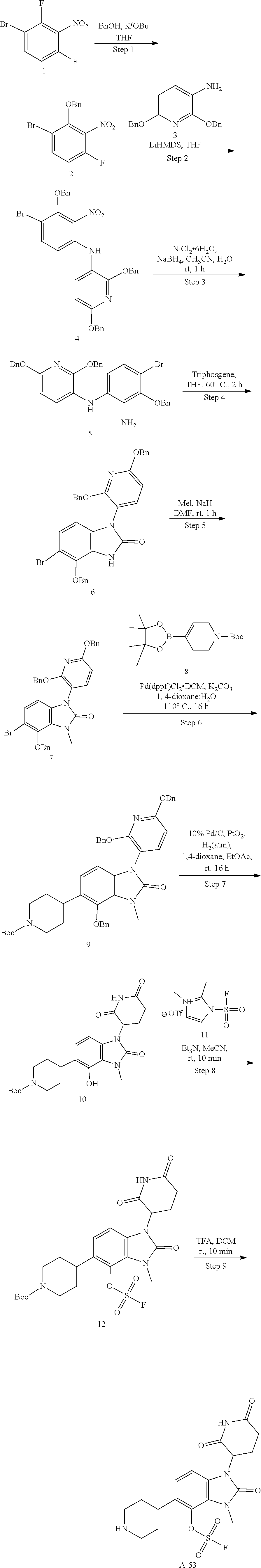

Step-1:

To a stirred solution of 1-bromo-2,4-difluoro-3-nitro-benzene 1 (5 g, 21.0 mmol) in tetrahydrofuran (103 mL) was added BnOH (2.2 mL, 21.0 mmol) at 0° C. and stirred at room temperature for 5 minutes. The reaction mixture was diluted with water, extracted with ethyl acetate (2×50 mL) and the organic layer was washed with saturated sodium chloride (2×50 mL) and dried over anhydrous sodium sulfate then evaporated under reduced pressure to afford 10 crude product, which was purified by combi-flash column chromatography (230-400 mesh silica gel, using 2% ethyl acetate in Hexane in eluent) to afford 2-benzyloxy-1-bromo-4-fluoro-3-nitro-benzene 2 (3.5 g, 49% yield) as a brown solid. LCMS (ES): m z 324.07 [M-H]-.

Step-2: To a stirred solution of 2,6-dibenzyloxypyridin-3-amine 3 (7 g, 22.9 mmol) in tetrahydrofuran (70 mL) was added LiHMDS (1.0 M, 34.3 mL) stirred at −78° C. for 1 hour. After, 2-benzyloxy-1-bromo-4-fluoro-3-nitro-benzene 2 (7.5 g, 22.9 mmol) was added and the reaction mixture was stirred at room temperature for 5 hours. The reaction mixture was quenched with ammonium chloride solution (40 mL) and extracted with ethyl acetate (2×40 mL) and dried over anhydrous sodium sulfate, the organic layer was concentrated under reduced pressure to afford the crude product, which was purified by combi flash column chromatography (Silica gel 230-400 mesh, using 20% ethyl acetate in Hexane) to afford 2,6-dibenzyloxy-N-(3-benzyloxy-4-bromo-2-nitro-phenyl)pyridin-3-amine 4 (2.2 g, 14% yield) as an yellow liquid. LCMS (ES*): m z 610.32 [M-H]-.

Step-3: To a stirred solution of 2,6-dibenzyloxy-N-(3-benzyloxy-4-bromo-2-nitro-phenyl)pyridin-3-amine 4 (1.1 g, 1.80 mmol) in acetonitrile (20.1 mL), water (2.0 mL) was added dichloronickelhexahydrate (85 mg, 359 mol) at 0° C. and stirred for 10 minutes. After, NaBH4 (136 mg, 3.6 mmol) was added at 0° C. and stirred at room temperature for 1 hour. The reaction mixture was quenched with ammonium chloride solution (20 mL) and extracted with ethyl acetate (2×30 mL), the organic layer dried over anhydrous sodium sulfate concentrated under reduced pressure to obtained crude compound, which was triturated with diethyl ether and dried under vacuum to afford 3-benzyloxy-4-bromo-Ni-(2,6-dibenzyloxy-3-pyridyl)benzene-1,2-diamine 5 (1.1 g, 89% yield) as an brown color solid. LCMS (ES*): m z 582.35 [M+H]+.

Step-4: To a stirred solution of 3-benzyloxy-4-bromo-Ni-(2,6-dibenzyloxy-3-pyridyl)benzene-1,2-diamine 5 (1 g, 1.7 mmol) in tetrahydrofuran (20 mL) was added triphosgene (510 mg, 1.7 mmol) and stirred at 60° C. for 2 hours. The reaction mixture was quenched with water (50 mL) and extracted with ethyl acetate (2×50 mL) the organic layer was washed with saturated sodium chloride solution (30 mL) and dried over anhydrous sodium sulfate the combined organic layer was concentrated under reduced pressure to afford crude product, which was purified by combi-flash column chromatography (Silica gel 230-400 mesh, using 80% ethyl acetate in Hexane as an eluent) to afford 7-benzyloxy-6-bromo-3-(2,6-dibenzyloxy-3-pyridyl)-1H-benzimidazol-2-one 6 (1 g, 94% yield) as a brown solid. LCMS (ES+): m z 608.24 [M-H]-. Step-5: To a stirred solution of 7-benzyloxy-6-bromo-3-(2,6-dibenzyloxy-3-pyridyl)-1H-benzimidazol-2-one 6 (1 g, 1.64 mmol) in dimethylformamide (5 mL) was added NaH (197 mg, 8.2 mmol) at 0° C. and stirred for 10 minutes. Next, methyl iodide (113 L, 1.81 mmol) was added and the reaction was stirred at room temperature for 1 hour. The reaction mixture was quenched with ammonium chloride solution (40 mL), extracted with ethyl acetate (2×40 mL). The organic layer was washed with saturated sodium chloride solution (30 mL), dried with anhydrous sodium sulfate and concentrated under reduced pressure to afford the crude product. The crude product was purified by combi-flash column chromatography (Silica gel 230-400 mesh, using 80% ethyl acetate in Hexane as an eluent) to afford 4-benzyloxy-5-bromo-1-(2,6-dibenzyloxy-3-pyridyl)-3-methyl-benzimidazol-2-one 7 (1 g, 66% yield) as a brown solid. LCMS (ES*): m z 622.41 [M+H]+.

Step-6: To a stirred solution of 7-benzyloxy-6-bromo-3-(2,6-dibenzyloxy-3-pyridyl)-1H-benzimidazol-2-one 7 (2 g, 3.3 mmol) and tert-butyl 4-(4,4,5,5-tetramethyl-1,3,2-dioxaborolan-2-yl)-3,6-dihydro-2H-pyridine-1-carboxylate 8 (1.52 g, 4.93 mmol) in 1,4-dioxane (20 mL) and water (5 mL) was added K2CO3 (909 mg, 6.57 mmol). After, the reaction mixture was degassed with N2 for 10 minutes and Pd(dppf)C12 DCM (268.42 mg, 328.69 mol) was added, and the reaction was stirred at 100° C. for 16 hours. The reaction mixture was diluted 15 with water, extracted with ethyl acetate (2×50 mL), washed with saturated sodium chloride solution (2×50 mL), dried over anhydrous sodium sulfate and evaporated under reduced pressure to afford the crude product. The crude product was purified by combi flash column chromatography (Silica gel 230-400 mesh, using 80% ethyl acetate in Hexane as an eluent) to afford tert-butyl 4-[4-benzyloxy-1-(2,6-dibenzyloxy-3-pyridyl)-3-methyl-2-oxo-benzimidazol-5-yl]-3,6-dihydro-2H-pyridine-1-carboxylate 9 (1.3 g, 48% yield) as a brown solid. LCMS (ES+): m z 725.74 [M-H]-.

Step-7: To a stirred solution of tert-butyl 4-[4-benzyloxy-1-(2,6-dibenzyloxy-3-pyridyl)-3-methyl-2-oxo-benzimidazol-5-yl]-3,6-dihydro-2H-pyridine-1-carboxylate 9 (1.3 g, 1.8 mmol) in tetrahydrofuran (20 mL) were added 10% palladium on carbon (wet with ca. 55% water) (954.32 mg, 8.97 mmol) and platinum(IV) oxide (203.63 mg, 897 mol). The reaction mixture was stirred at room temperature for 16 hours under H2 atmosphere (parr shaker, 80 psi). Upon completion of the reaction, the reaction mixture was filtered through a celite pad and washed with ethyl acetate (40 mL). The filtrate was evaporated under reduced pressure to afford the crude product, which was triturated with diethyl ether (50 mL) and dried under reduced pressure to afford tert-butyl 4-[1-(2,6-dioxo-3-piperidyl)-4-hydroxy-3-methyl-2-oxo-benzimidazol-5-yl]piperidine-1-carboxylate 10 (0.6 g, 59% yield) as a brown solid. LCMS (ES+): m z 457.34 [M-H]-.

Step-8: To a stirred solution of tert-butyl 4-[1-(2,6-dioxo-3-piperidyl)-4-hydroxy-3-methyl-2-oxo-benzimidazol-5-yl]piperidine-1-carboxylate 10 (0.1 g, 218.10 mol) in acetonitrile (3 mL) were added triethylamine (66.2 mg, 654.30 mol) at 0° C. followed by 1-(fluorosulfonyl)-2,3-dimethyl-1H-imidazol-3-ium trifluoromethanesulfonate 11 (93 mg, 284 mol) and stirred at room temperature for 10 minutes. Upon completion of the reaction, the reaction mixture was diluted with water, extracted with ethyl acetate (2×10 mL), washed with saturated sodium chloride solution (2×20 mL), dried over anhydrous sodium sulfate and evaporated under reduced pressure to afford the crude product. The crude product was purified by combi flash column chromatography (Silica gel 230-400 mesh, using 50% ethyl acetate in Hexane as an eluent) to afford tert-butyl 4-[1-(2,6-dioxo-3-piperidyl)-4-fluorosulfonyloxy-3-methyl-2-oxo-benzimidazol-5-yl]piperidine-1-carboxylate 12 (0.03 g, 24% yield) as a green solid. LCMS (ES): m z 539.20 [M-H]-.

Step-9: To a stirred solution of tert-butyl 4-[1-(2,6-dioxo-3-piperidyl)-4-fluorosulfonyloxy-3-methyl-2-oxo-benzimidazol-5-yl]piperidine-1-carboxylate 12 (0.03 g, 56 mol) in dichloromethane (3 mL) was added trifluoroacetic acid (12.7 pL, 166 mol) at 0° C. and stirred at room temperature for 10 minutes. The reaction mixture was evaporated under reduced pressure to afford the crude product, which was triturated with diethyl ether (10 mL) and dried 20 under reduced pressure to afford 1-(2,6-dioxo-3-piperidyl)-4-fluorosulfonyloxy-3-methyl-2-oxo-5-(4-piperidyl)benzimidazole (A-53) (23 mg, 73% yield, trifluoroacetic acid) as a white solid. LCMS (ES): m/z 441.33 [M-H]-.¹H NMR (400 MHz, DMSO-d6) 6:11.17 (s, 1H), 7.35 (d, J=8.40 Hz, 1H), 7.07 (d, J=8.40 Hz, 1H), 5.48-5.44 (m, 1H), 3.52 (s, 3H), 3.46-3.45 (m, 2H), 3.27-3.23 (m, 1H), 3.20-3.15 (m, 2H), 2.95-2.87 (m, 1H), 2.75-2.70 (m, 1H), 2.68-2.62 (m, 1H), 2.09-2.06 (m, 1H), 1.96 (s, 4H).

Synthesis A52: Synthesis of 3-(2,6-dioxopiperidin-3-yl)imidazo[1,2-a]pyridin-8-yl sulfurofluoridate (A-54)

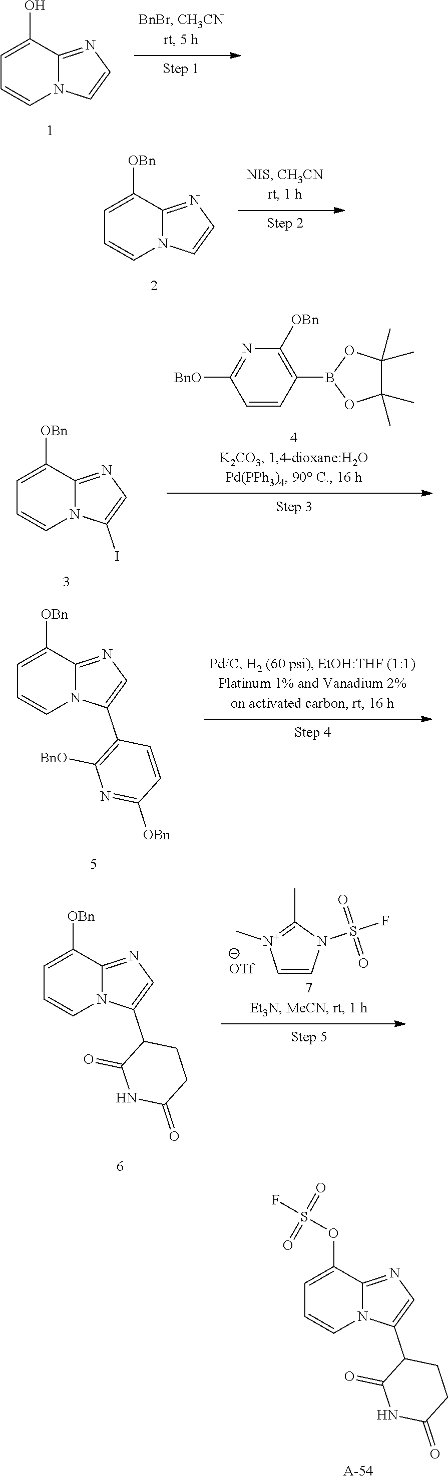

Step-1:

To a solution of imidazo[1,2-a]pyridin-8-ol 1 (2.50 g, 18.64 mmol) in acetonitrile (50 5 mL) were added potassium carbonate (2.58 g, 18.6 mmol), bromomethylbenzene (2.22 mL, 18.64 mmol) and stirred at room temperature for 5 hours. Upon completion of the reaction, the reaction mixture was evaporated under reduced pressure to obtain the crude product. The crude product was purified by column chromatography (Davisil silica gel, using 50% ethyl acetate and pet-ether as eluent) to afford 8-benzyloxyimidazo[1,2-a]pyridine 2 (800 mg, 18% yield) as 10 a clear oil. LCMS (ES*): m z 225.30 [M+H]+.

Step-2: To a stirred solution of 8-benzyloxyimidazo[1,2-a]pyridine 2 (800 mg, 3.6 mmol) in acetonitrile (20 mL) was added N-iodosuccinimide (803 mg, 3.6 mmol) at 0° C. and stirred at room temperature for 1 hour. Upon completion of the reaction, reaction mixture was diluted 15 with ice- chilled water (100 mL) and extracted with ethyl acetate (2×50 mL). The organic layer was dried over anhydrous sodium sulfate, filtered, and evaporated under reduced pressure to afford 8-benzyloxy-3-iodo-imidazo[1,2-a]pyridine 3 (1.0 g, 72% yield) as a pale-yellow solid. LCMS (ES+): m z 351.31 [M+H]+.

Step-3: To a solution of 8-benzyloxy-3-iodo-imidazo[1,2-a]pyridine 3 (1.0 g, 2.9 mmol) and 2,6-dibenzyloxy-3-(4,4,5,5-tetramethyl-1,3,2-dioxaborolan-2-yl)pyridine 4 (953 mg, 2.3 mmol) in 1,4-dioxane (8 mL), water (2 mL) was added potassium carbonate (1.2 g, 8.6 mmol) and degassed with N2 for 5 minutes before adding Pd(PPh3)4 (330 mg, 286 mol). The reaction mixture was stirred at 100° C. for 16 hours. Upon completion of the reaction, reaction mixture was evaporated under reduced pressure to obtain crude product which was purified by column chromatography (Davisil silica gel, using 30% ethyl acetate and petroleum ether as eluent) to afford 8-benzyloxy-3-(2,6-dibenzyloxy-3-pyridyl) imidazo[1,2-a]pyridine 5 (900 mg, 45% yield) as a brown solid. LCMS (ES*): m z 514.50 [M+H]+Step-4: To a stirred solution of 8-benzyloxy-3-(2,6-dibenzyloxy-3-pyridyl) imidazo[1,2-a]pyridine 5 (550 mg, 1.1 mmol) in tetrahydrofuran (10 mL), ethanol (10 mL) were added Pd/C (20% on carbon (wetted with ca. 55% Water)) (455.86 mg, 4.28 mmol), platinum 1% and vanadium 2% on activated carbon (200 mg, 2.14 mmol) at room temperature and stirred under H2 (60 psi) for 16 hours. Upon completion of the reaction, reaction mixture was filtered through a celite plug and washed with tetrahydrofuran (30 mL), acetonitrile (30 mL), ethanol (30 mL).

The filtrate was evaporated under reduced pressure to obtain crude product 3-(8-hydroxyimidazo[1,2-a]pyridin-3-yl) piperidine-2,6-dione 6 (130 mg, 38% yield) as a brown solid. LCMS (ES*): m z 246.28 [M+H]+.

Step-5: To a stirred solution of 3-(8-hydroxyimidazo[1,2-a]pyridin-3-yl)piperidine-2,6-dione 6 (130 mg, 530.11 mol) in acetonitrile (5 mL) were added triethylamine (296 L, 2.1 mmol) and 1-(fluorosulfonyl)-2,3-dimethyl-1H-imidazol-3-ium trifluoromethanesulfonate 7 (524 mg, 1.6 mmol) at room temperature and the reaction was stirred for 1 hour. Upon completion of the reaction, the reaction mixture was diluted with water (15 mL) and extracted using ethyl acetate (2×10 mL). The organic layer was dried over anhydrous sodium sulfate and evaporated under reduced pressure to obtain crude product. The crude product was purified by preparative HPLC [Column: Sun Fire C18, 19×150 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 3-(2,6-dioxo-3-piperidyl)-8-fluorosulfonyloxy-imidazo[1,2-a]pyridine (A-54) (22 mg, 9% yield, trifluoroacetic acid) as a brown oil. LCMS (ES*): m z 328.20 [M+H]+. H-NMR (400 MHz, DMSO-d6): δ 11.01 (s, 1H), 8.55 (d, J=6.8 Hz, 1H), 7.69 (d, J=7.6 Hz, 1H), 7.63 (s, 1H), 7.07 (s, 1H), 4.55 (dd, J=4.4 Hz, 1H), 2.79-2.65 (m, 2H), 2.60-2.55 (m, 1H), 2.24-2.20 (m, 1H).

Synthesis A53: Synthesis of 5-((1-methyl-2,6-dioxopiperidin-3-yl)amino)-2-(piperidin-4-yl)phenyl sulfurofluoridate (A-55)

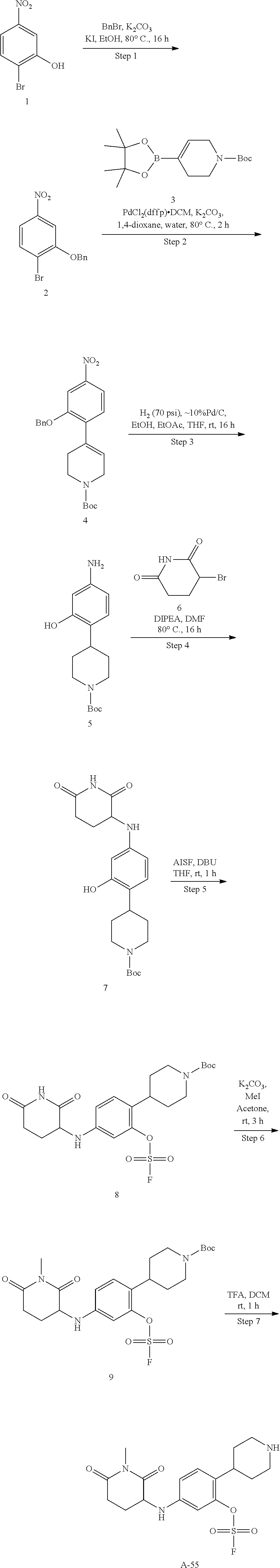

Step-1: A stirred solution of 2-bromo-5-nitro-phenol 1 (30 g, 137.61 mmol) in ethanol (100 mL) was added potassium carbonate (22.82 g, 165 mmol) and stirred for 2 hours at 90° C.

Benzyl bromide (21.28 mL, 178.89 mmol) and potassium iodide (11.42 g, 68.81 mmol) were added into the reaction mixture and was stirred at 90° C. for 16 hours. Upon completion of the reaction, the reaction mixture evaporated under reduced pressure and diluted with water (900 mL) and extracted with ethyl acetate (3×350 mL). The combined organic layers were dried over sodium sulfate and concentrated under reduced pressure to afford the crude product. The crude product was purified by column chromatography (Davisil silica gel, using 10% ethyl acetate in petroleum ether as an eluent) to afford 2-benzyloxy-1-bromo-4-nitro-benzene 2 (41 g, 94% yield) as a pale-yellow solid. LCMS (ES-): m z 307.24 [M-H]-.

Step-2: To a stirred solution of 2-benzyloxy-1-bromo-4-nitro-benzene 2 (12 g, 38.94 mmol), tert-butyl 4-(4,4,5,5-tetramethyl-1,3,2-dioxaborolan-2-yl)-3,6-dihydro-2H-pyridine-1-carboxylate 3 (13.25 g, 42.84 mmol) in 1,4-dioxane (85 mL) and water (15 mL) was added potassium carbonate (16.15 g, 116.83 mmol) at room temperature. The resulting mixture was degassed with argon gas for 10 minutes. Next, Pd(dppf)C12 DCM (2.54 g, 3.12 mmol) was added to the reaction mixture and again the reaction was degassed with argon for 5 minutes, the resulting reaction mixture was stirred at 80° C. for 2 hours. Upon completion of the reaction, the reaction mixture was filtered through a celite bed and washed with ethyl acetate (200 mL), the obtained filtrate was concentrated under reduced pressure to afford the crude product. The crude product was purified by column chromatography (silica gel 230-400 mesh, using 30% ethyl acetate in petroleum ether as eluent) to afford tert-butyl 4-(2-benzyloxy-4-nitro-phenyl)-3,6-dihydro-2H-pyridine-1-carboxylate 4 (8.5 g, 45% yield) as a yellow oil. LCMS (ES*): m z 311.25 [M-Boc+H]+.

Step-3: To a stirred solution of tert-butyl 4-(2-benzyloxy-4-nitro-phenyl)-3,6-dihydro-2H-pyridine-1-carboxylate 4 (13 g, 31.67 mmol) in tetrahydrofuran (30 mL), ethanol (30 mL) and ethyl acetate (40 mL) was added 10% palladium on carbon (wetted with ca. 55% Water) (3.37 g, 31.67 mmol) and stirred for 16 hours at room temperature under H2 (70 psi). Upon completion of the reaction, the reaction mixture was filtered through a celite bed and washed with dichloromethane (200 mL), the obtained filtrate was concentrated under reduced pressure to afford crude product tert-butyl 4-(4-amino-2-hydroxy-phenyl)piperidine-1-carboxylate 5 (9.36 g, 45% yield) as a brown solid. LCMS (ES-): m z 291.22 (M-H)-.

Step-4: To a stirred solution of tert-butyl 4-(4-amino-2-hydroxy-phenyl) piperidine-1-carboxylate 5 (8 g, 27.36 mmol) in dimethylformamide (40 mL) was added N,N-diisopropylethylamine (14.30 mL, 82.1 mmol) and stirred for 10 minutes. After, 3-bromopiperidine-2,6-dione 6 (7.88 g, 41 mmol) was added into the reaction mixture and the reaction was stirred at 80° C. for 16 hours. Upon completion of the reaction, the reaction was quenched with water (300 mL) and extracted with ethyl acetate (3×100 mL). The combined organic layers were washed with saturated sodium chloride solution, dried over anhydrous sodium sulfate and concentrated under reduced pressure to afford the crude product. The crude product was purified by column chromatography (Silica gel 60-120 mesh, using 60% ethyl acetate in petroleum ether as eluent) to afford tert-butyl 4-[4-[(2,6-dioxo-3-piperidyl)amino]-2-hydroxy-phenyl]piperidine-1-carboxylate 7 (1.6 g, 11% yield) as a brown solid. LCMS (ES-): m z 402.37 [M-H]-.

Step-5: To a stirred solution of tert-butyl 4-[4-[(2,6-dioxo-3-piperidyl)amino]-2-hydroxy-phenyl]piperidine-1-carboxylate 7 (1.3 g, 3.22 mmol) in tetrahydrofuran (13 mL) was added DBU (1.20 mL, 8.06 mmol) and 4-acetamidobenzenesulfonyl azide (928.88 mg, 3.87 mmol) at room temperature and stirred at room temperature for 1 hour. The reaction mixture was concentrated under reduced pressure to afford the crude product. The crude product was purified by column chromatography (Silica gel 60-120 mesh, using 60% ethyl acetate in petroleum ether as eluent) to afford tert-butyl 4-[4-[(2,6-dioxo-3-piperidyl)amino]-2-fluorosulfonyloxy-phenyl]piperidine-1-carboxylate 8 (0.53 g, 29% yield) as an off-white solid.

LCMS (ES-): m z 430.34 [M-tBu+H]+.¹H NMR (400 MHz, DMSO-d6) 6:10.88 (s, 1H), 7.21 (d, J=9.2 Hz, 1H), 6.75 (d, J=6.4 Hz, 2H), 6.37 (d, J=8.0 Hz, 1H), 4.35-4.41 (m, 1H), 4.05-4.08 (m, 2H), 2.69-2.78 (m, 4H), 2.59-2.60 (m, 1H), 2.06-2.10 (m, 1H), 1.90-1.95 (m, 1H), 1.85-1.88 (m, 2H), 1.63 (m, 11H).

Step-6: To a stirred solution of tert-butyl 4-[4-[(2,6-dioxo-3-piperidyl)amino]-2-fluorosulfonyloxy-phenyl]piperidine-1-carboxylate 8 (0.2 g, 411.93 mol) in acetone (4 mL) was added potassium carbonate (113.86 mg, 823.85 mol) and stirred for 5 minutes. Next, iodomethane (30.77 L, 494.31 mol) was added into the reaction mixture at 0° C. and stirred at room temperature for 3 hours. Upon completion, the reaction mixture was diluted with water (2×2 OmL) and extracted with ethyl acetate (2×25 mL), the organic layer was dried over sodium sulfate and concentrated under reduced pressure to afford the crude product. The crude product was purified by preparative HPLC [Column: X-SELECT C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[2-fluorosulfonyloxy-4-[(1-methyl-2,6-dioxo-3-piperidyl) amino]phenyl]piperidine-1-carboxylate 9 (0.1 g, 44% yield) as a colorless oil. LCMS (ES-): m z 402.37 [M-H]-.

Step-7: To a stirred solution of tert-butyl 4-[2-fluorosulfonyloxy-4-[(1-methyl-2,6-dioxo-3-piperidyl) amino]phenyl]piperidine-1-carboxylate 9 (0.1 g, 200.18 mol) in dichloromethane 10 (2 mL) was added trifluoroacetic acid (273.90 mg, 2.40 mmol) at 0° C. The reaction mixture was stirred at 0° C. for 1 hour. Upon completion of the reaction, the reaction mixture was concentrated under reduced pressure to afford the crude product. The residue was triturated with diethyl ether (20 ml) to afford 3-[3-fluorosulfonyloxy-4-(4-piperidyl) aniline]-1-methyl-2,6-dioxo-piperidine (A-55) (71.0 mg, 69% yield) as an off-white solid. LCMS (ES*): m z 15 400.41[M+H]+. ¹H NMR (400 MHz, DMSO) 6: 7.13 (d, J=8.40 Hz, 1H), 6.81 (q, J=8.40 Hz, 2H), 6.52 (d, J=8.OHz, 1H), 4.33 (q, J=4.4 Hz, 1H), 3.26 (s, 2H), 3.11-3.05 (m, 3H), 3.00 (s, 3H), 2.90-2.75 (m, 2H), 2.15-2.05 (m, 1H), 2.00-1.90 (m, 1H), 1.85-1.75 (m, 4H).

Synthesis A54: Synthesis of 5-(2,6-dioxopiperidin-3-yl)-2-(piperazin-1-yl)benzenesulfonyl 20 fluoride (A-56)

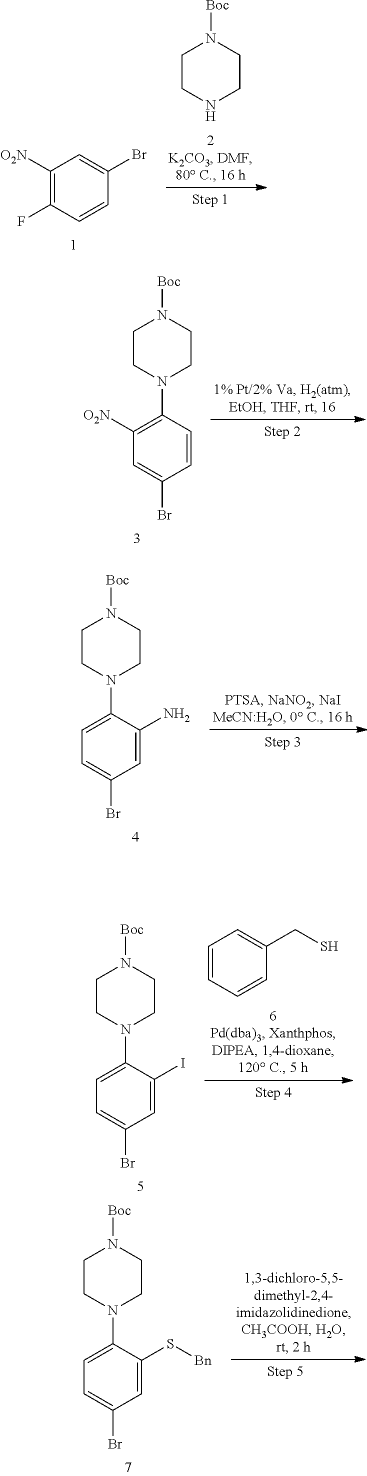

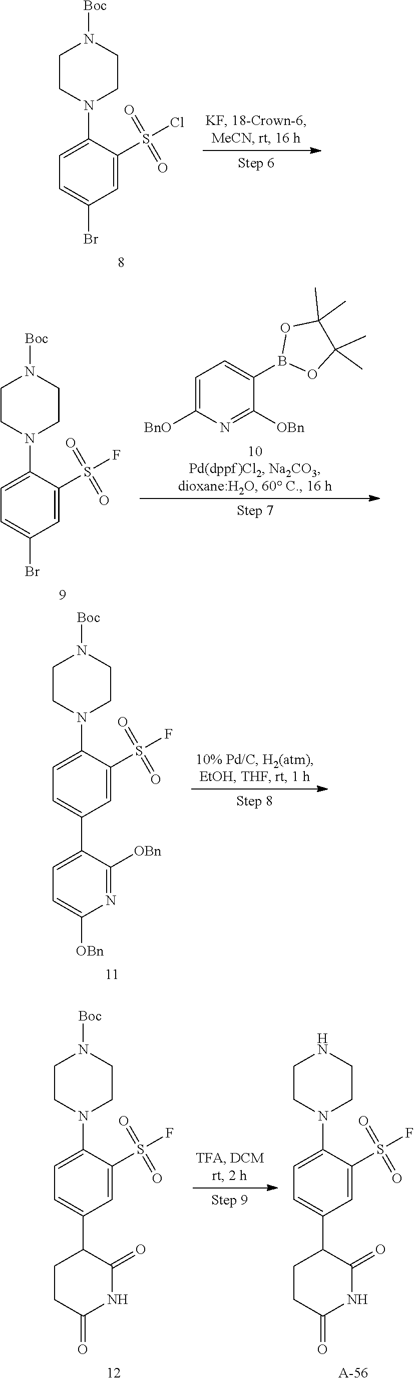

To a stirred solution of 4-bromo-1-fluoro-2-nitro-benzene 1 (15 g, 68.18 mmol) and tert-butyl piperazine-1-carboxylate 2 (10.16 g, 54.55 mmol) in dimethylformamide (50 mL) were added potassium carbonate (23.56 g, 170.46 mmol) at room temperature and stirred at 80° C. for 16 hours. Upon completion of the reaction, the reaction mixture was diluted with water (500 mL) and extracted with ethyl acetate (3×200 mL). The combined organic layer was 10 washed with saturated sodium chloride solution (200 mL), dried over anhydrous sodium sulfate, and concentrated under reduced pressure to afford the crude product. The crude product was purified by column chromatography (Davisil silica gel, using 10% ethyl acetate in petroleum ether as a eluent) to afford tert-butyl 4-(4-bromo-2-nitro-phenyl)piperazine-1-carboxylate 3 (12 g, 40% yield) as a yellow solid. LCMS (ES*): m z 286.14 [M-Boc+H]+.

Step-²: To a stirred solution of tert-butyl 4-(4-bromo-2-nitro-phenyl)piperazine-1-carboxylate 3 (5 g, 12.95 mmol) in methanol (30 mL) and dichloromethane (70 mL) was added 1% platinum and 2% vanadium on activated carbon (5 g, 12.95 mmol) at room temperature. The reaction mixture was stirred under H2 atmosphere (hydrogen balloon) for 16 hours. Upon completion of reaction, the reaction mixture was filtered through a celite bed and washed with 10 dichloromethane (50 mL). The filtrate was concentrated under reduced pressure to afford tert-butyl 4-(2-amino-4-bromo-phenyl)piperazine-1-carboxylate 4 (4.5 g, 82% yield) as a solid.

This was directly taken into the next step without further purification. LCMS (ES+): m z 356.25 [M+H]+.

Step-3: To a stirred solution of tert-butyl 4-(2-amino-4-bromo-phenyl)piperazine-1-carboxylate 4 (4.5 g, 12.63 mmol) in acetonitrile (45 mL), water (5 mL) were added sodium nitrite (803.97 pL, 25.26 mmol) and p-Toluene sulphonic acid (4.81 g, 27.91 mmol) at 0° C. and stirred for 30 minutes. Next, sodium iodide (3.79 g, 25.26 mmol) was added to the reaction mixture and stirred at 25° C. for 16 hours. Upon completion of reaction, the reaction mixture 20 was quenched with water (200 mL) and extracted with ethyl acetate (2×200 mL). The combined organic layer was washed with saturated sodium chloride solution (100 mL), dried over anhydrous sodium sulfate and concentrated under reduced pressure to afford the crude compound, which was purified by column chromatography (Davisil silica gel, using 20% ethyl acetate in petroleum ether as a eluent) to afford tert-butyl 4-(4-bromo-2-iodo-phenyl)piperazine-1-carboxylate 5 (4.0 g, 61% yield) as an off-white solid. LCMS (ES+): m z 411.22 [M-^{t}Bu+H]+.

Step-4: To stirred solution of tert-butyl 4-(4-bromo-2-iodo-phenyl)piperazine-1-carboxylate 5 (0.5 g, 1.07 mmol) and phenylmethanethiol 6 (106.35 mg, 856.28 mol) in 1,4 dioxane (5 mL) was added diisopropylethylamine (415.00 mg, 3.21 mmol, 559.30 L) at room temperature and degassed with argon gas for 10 minutes. After, Pd2(dba)3 (98.01 mg, 107.03 mol) and Xanthphos (123.86 mg, 214.07 mol) were added, and the reaction mixture was stirred at 120° C. for 5 hours. Upon completion, the reaction mixture was quenched with water (200 mL) and extracted with ethyl acetate (3×200 mL). The combined organic layer was washed with saturated sodium chloride solution (100 mL), dried over anhydrous sodium sulfate and concentrated under reduced pressure to afford the crude product, which was purified by column chromatography (Davisil silica gel, using 20% ethyl acetate in petroleum ether as a eluent) to afford tert-butyl 4-(2-benzylsulfanyl-4-bromo-phenyl)piperazine-1-carboxylate 7 (0.4 g, 69% yield) as an off-white LCMS (ES*): m z 407.32 [M-^{t}Bu+H]+.

Step-5: To a stirred solution of tert-butyl 4-(2-benzylsulfanyl-4-bromo-phenyl)piperazine-1-carboxylate 7 (400 mg, 863.13 mol) in acetonitrile (8 mL), acetic acid (1 mL) and water (1 mL) was added 1,3-dichloro-5,5-dimethyl-2,4-imidazolidinedione (226.74 L, 1.73 mmol) at 0° C. and stirred at room temperature for 2 hours. Upon completion of the reaction, the reaction mixture was diluted with water (100 mL) and extracted with ethyl acetate (2×50 mL), and the combined organic layers were dried over sodium sulfate and concentrated under reduced pressure to afford tert-butyl 4-(4-bromo-2-chlorosulfonyl-phenyl)piperazine-1-carboxylate 8 (0.350 g, 46% yield) as a colorless oil. LCMS (ES-): m z 421.33 [M-17]+. Note: Crude taken to next reaction without further purification.

Step-6: To a stirred solution of tert-butyl 4-(4-bromo-2-chlorosulfonyl-phenyl)piperazine-1-carboxylate 8 (0.460 g, 1.05 mmol) in acetonitrile (5 mL) were added potassium fluoride (121.54 mg, 2.09 mmol) and 18-crown-6 (23.53 L, 104.60 mol) at 0° C. and stirred at room temperature for 16 hours. Upon completion of the reaction, the reaction mixture was diluted with water (50 mL) and extracted with ethyl acetate (2×50 mL), The combined organic layers were dried over sodium sulfate, filtered and concentrated under reduced pressure to afford the crude product, which was purified by column chromatography (Davisil silica gel, using 20% Ethyl acetate in petroleum ether as a eluent) to afford tert-butyl 4-(4-bromo-2-fluorosulfonyl-phenyl)piperazine-1-carboxylate 9 (0.12 g, 16% yield) as an off-white solid. LCMS (ES+): m z 367.14 [M-tBu+H]+.

Step-7: To stirred solution of tert-butyl 4-(4-bromo-2-fluorosulfonyl-phenyl)piperazine-1-carboxylate 9 (120 mg, 283.49 mol) and 2,6-dibenzyloxy-3-(4,4,5,5-tetramethyl-1,3,2-dioxaborolan-2-yl)pyridine 10 (130.13 mg, 311.84 mol) in 1,4-dioxane (1.6 mL) and water (0.4 mL) was added sodium carbonate (60.09 mg, 566.98 mol, 23.73 L), the reaction mixture was degassed with N2 for 10 minutes, then was added Pd(dppf)Cl2 (21 mg, 28.4 mol) at room temperature. The reaction mixture was stirred at 60° C. for 16 hours. Upon completion of the reaction, the reaction mixture was diluted with water (50 mL) and extracted with ethyl acetate (2×50 mL). The combined organic layer was dried over anhydrous sodium sulfate and concentrated under reduced pressure to afford the crude compound, which was purified by column chromatography (Davisil silica gel, using 30% Ethyl acetate in petroleum ether as a eluent) to afford tert-butyl 4-[4-(2,6-dibenzyloxy-3-pyridyl)-2-fluorosulfonyl-phenyl]piperazine-1-carboxylate 11 (100 mg, 50% yield) as an off-white solid. LCMS (ES*): m z 578.38 [M-^{t}Bu+H]+.

Step-8: To a stirred solution of tert-butyl 4-[4-(2,6-dibenzyloxy-3-pyridyl)-2-fluorosulfonyl-phenyl]piperazine-1-carboxylate 11 (100 mg, 157.80 mol) in tetrahydrofuran (2 mL) and ethyl acetate (2 mL), 10% palladium on carbon (wetted with ca. 55% Water) (84 mg, 789 mol) was added and stirred at room temperature for 1 hour under a H2 atmosphere (balloon). Upon completion of the reaction, the reaction mixture was concentrated under reduced pressure to afford tert-butyl 4-[4-(2,6-dioxo-3-piperidyl)-2-fluorosulfonyl-phenyl]piperazine-1-carboxylate 12 (0.05 g, 67% yield) as an off-white solid. The material was directly used in the next step without further purification. LC-MS (ES-): m z 454.35 [M-H]-.

Step-9: To a stirred solution of tert-butyl 4-[4-(2,6-dioxo-3-piperidyl)-2-fluorosulfonyl-phenyl]piperazine-1-carboxylate 12 (0.05 g, 110 mol) in dichloromethane (2 mL), trifluoroacetic acid (84 pL, 1.10 mmol) was added dropwise at 0° C. and stirred at room temperature for 2 hours. Upon completion, the reaction mixture was concentrated under reduced pressure to afford the crude product, which was triturated with diethyl ether (2×10 mL) and dried under reduced pressure to afford 5-(2,6-dioxo-3-piperidyl)-2-piperazin-1-yl-benzenesulfonyl fluoride (A-56) (15.2 mg, 29% yield, trifluoroacetate salt) as an off-white solid. LCMS (ES-): m z 356.21 [M+H]+. ¹H NMR (400 MHz, DMSO-d6) δ: 10.93 (s, 1H), 8.71 (s, 1H), 7.95 (s, 1H), 7.82 (d, J=8.00 Hz, 1H), 7.71 (d, J=8.40 Hz, 1H), 4.12 (dd, J=4.40, 12.60 Hz, 1H), 3.23 (d, J=2.00 Hz, 4H), 3.15 (d, J=4.80 Hz, 4H), 2.72-2.66 (m, 1H), 2.58 (d, J=4.40 Hz, 1H), 2.34-2.27 (m, 1H), 2.04-2.02 (m, 1H).

Synthesis A55: Synthesis of 3-(2,6-dioxopiperidin-3-yl)benzo[d]isoxazole-7-sulfonyl fluoride (A-58)

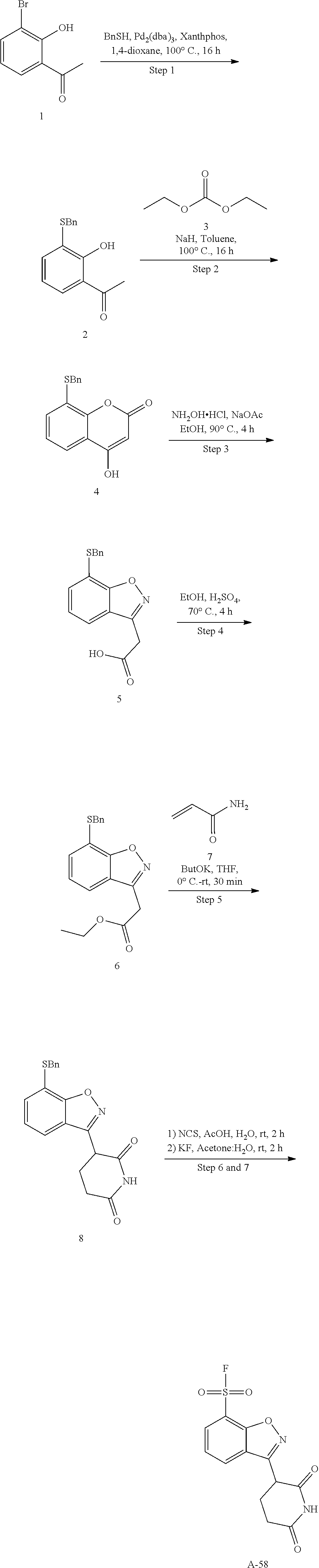

Step-1: To a stirred solution of 1-(3-bromo-2-hydroxy-phenyl)ethanone 1 (3 g, 14 mmol) and phenylmethanethiol (3.3 mL, 28 mmol) in 1,4-dioxane (30 mL) were added N,N-diisopropylethylamine (7.3 mL, 42 mmol), Xanthphos (807 mg, 1.40 mmol) and purged with 10 argon gas for 5 minutes. Next, Pd2(dba)3 (1.28 g, 1.40 mmol) was added to the reaction and stirred at 100° C. for 16 hours. Upon completion of the reaction, the reaction mixture was diluted with water (50 mL) and extracted with ethyl acetate (3×200 mL). The combined organic layer was washed with saturated sodium chloride solution (30 mL), dried over anhydrous sodium sulfate and evaporated under reduced pressure to afford the crude product. The crude product was further purified by column chromatography (Davisil silica gel, using ~2% ethyl acetate in petroleum ether as an eluent) to afford 1-(3-benzylsulfanyl-2-hydroxy-phenyl)ethanone 2 (4 g, 91% yield) as a brown solid. LCMS (ES-): m z 257.17 [M-H]-.

Step-2: To a stirred solution of 1-(3-benzylsulfanyl-2-hydroxy-phenyl)ethanone 2 (4 g, 15.48 mmol) in toluene (40 mL) was added NaH (60% dispersion in oil) (3.72 g, 155 mmol) at 0° C. and stirred for 15 minutes before adding diethyl carbonate 3 (7.50 mL, 61.94 mmol) at 0° C. The reaction was warmed to room temperature and stirred at 100° C. for 16 hours. Upon completion of the reaction, the reaction mixture was diluted water (100 mL) and extracted with ethyl acetate (3×250 mL). The aqueous layer was acidified with 1.5 N HCl, the precipitated solid was filtered and dried under reduced pressure to afford 8-benzylsulfanyl-4-hydroxy-chromen-2-one 4 (3 g, 55% yield) as an off-white solid. H-NMR (400 MHz, DMSO-d6): δ 12.27 (s, 1H), 7.73 (dd, J=8.80, Hz, 1H), 7.56 (dd, J=7.60 Hz, 1H), 7.35 (d, J=7.20 Hz, 2H), 7.29 (t, J=14.40 Hz, 2H), 7.20 (d, J=7.20 Hz, 1H), 6.94 (t, J=15.60 Hz, 1H), 4.26 (s, 2H).

Step-3: To a stirred solution of 8-benzylsulfanyl-4-hydroxy-chromen-2-one 4 (3 g, 10.55 mmol) in ethanol (40 mL) were added sodium acetate (2.60 g, 31.65 mmol), hydroxylamine hydrochloride (2.20 g, 31.65 mmol) and stirred at 90° C. for 4 hours. Upon completion of the reaction, the reaction mixture was diluted with water (30 mL) and extracted with ethyl acetate (2×100 mL). The combined organic layer was washed with saturated sodium chloride solution (20 mL), dried over anhydrous sodium sulfate and evaporated under reduced pressure to afford 2-(7-benzylsulfanyl-1,2-benzoxazol-3-yl)acetic acid 5 (3 g, 83% yield) as a brown solid.

LCMS (ES*): m z 300.01 [M+H]+.

Step-4: To a stirred solution of 2-(7-benzylsulfanyl-1,2-benzoxazol-3-yl)acetic acid 5 (2.95 g, 9.85 mmol) in ethanol (30 mL) was added H2SO4 (4 M, 4.93 mL) and stirred at 90° C. for 4 hours. Upon completion of the reaction, the reaction mixture was basified with an aqueous saturated sodium bicarbonate (250 mL) and extracted with ethyl acetate (2×500 mL). The combined organic layer was washed with saturated sodium chloride solution (50 mL), dried over anhydrous sodium sulfate and evaporated under reduced pressure to afford ethyl 2-(7-benzylsulfanyl-1,2-benzoxazol-3-yl)acetate 6 (2.9 g, 72% yield) as brown oil.H-NMR: (400 MHz, DMSO-d6) δ: 7.68 (dd, J=8.80 Hz, 1H), 7.60 (dd, J=8.40 Hz, 1H), 7.35-7.33 (m, 3H), 7.29-7.23 (m, 3H), 4.40 (s, 2H), 0.00 (s, 2H), 4.17-4.11 (m, 2H), 1.21-1.16 (m, 3H). Step-5: To a stirred solution of ethyl 2-(7-benzylsulfanyl-1,2-benzoxazol-3-yl)acetate 6 (0.5 g, 1.53 mmol) and prop-2-enamide 7 (164.22 L, 3.05 mmol) in tetrahydrofuran (5 mL) was added potassium tert-butoxide (205.64 mg, 1.83 mmol) at 0° C. and stirred at room temperature 5 for 30 minutes. Upon completion of the reaction, the reaction mixture was diluted with water (10 mL) and extracted with ethyl acetate (2×50 mL), the organic layer was washed with saturated sodium chloride solution (10 mL), dried over anhydrous sodium sulfate and concentrated under reduced pressure to afford the crude product, which was further purified by combi flash column chromatography (using Davisil silica gel, ~20% ethyl acetate in petroleum ether as a eluent) to afford 3-(7-benzylsulfanyl-1,2-benzoxazol-3-yl)piperidine-2,6-dione 8 (0.25 g, 43% yield) as an white solid. LCMS (ES-): m z 351.24 [M-H]-.

Step-6: To a stirred solution of 3-(7-benzylsulfanyl-1,2-benzoxazol-3-yl)piperidine-2,6-dione 8 (0.1 g, 283.76 mol) in acetonitrile (2 mL), acetic acid (0.2 mL) and water (0.2 mL) was added 1,3-dichloro-5,5-dimethyl-2,4-imidazolidinedione (75 L, 567.53 mol) and stirred at room temperature for 2 hours. Upon completion, the reaction mixture was diluted with water (10 mL) and extracted with ethyl acetate (2×30 mL), the organic layer was washed with saturated sodium chloride solution (5 mL) and dried over anhydrous sodium sulfate and concentrated under reduced pressure to afford crude 3-(2,6-dioxo-3-piperidyl)-1,2-benzoxazole-7-sulfonyl chloride 9 (0.1 g, 70% yield) as a brown oil, which was forwarded to next step without further purification. LCMS (ES-): m z 327.26 [M-H]-.

Step-7: To a stirred solution of 3-(2,6-dioxo-3-piperidyl)-1,2-benzoxazole-7-sulfonyl chloride 9 (0.1 g, 304.20 mol) in acetonitrile (3 mL) was added potassium fluoride (44.18 mg, 760.51 mol) and 18-crown-6 (6.84 L, 30.42 mol) portion wise at 0° C. portion and stirred at room temperature for 2 hours. Upon completion of the reaction, the reaction mixture was diluted with water (10 mL) and extracted with ethyl acetate (2×40 mL), the combined organic layer was washed with saturated sodium chloride solution (10 mL), dried over anhydrous sodium sulfate and concentrated under reduced pressure to afford crude product, which was purified by preparative HPLC [Column: UNISIL C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product 3-(2,6-dioxo-3-piperidyl)-1,2-benzoxazole-7-sulfonyl fluoride (A-58) (15.3 mg, 16% yield) as an off-white solid. LCMS (ES*): m z 313.19 [M+H]+. H-NMR (400 MHz, DMSO-d6): δ 11.19 (s, 1H), 8.53 (d, J=8.00 Hz, 1H), 8.41 (d, J=7.20 Hz, 1H), 7.76 (t, J=15.60 Hz, 1H), 4.81-4.77 (m, 1H), 2.84-2.75 (m, 1H), 2.68-2.56 (m, 2H), 2.33-2.23 (m, 1H).

Synthesis A56: Synthesis of (3-((2,6-dioxopiperidin-3-yl)oxy)phenyl)sulfamoyl fluoride 5 (A-59)

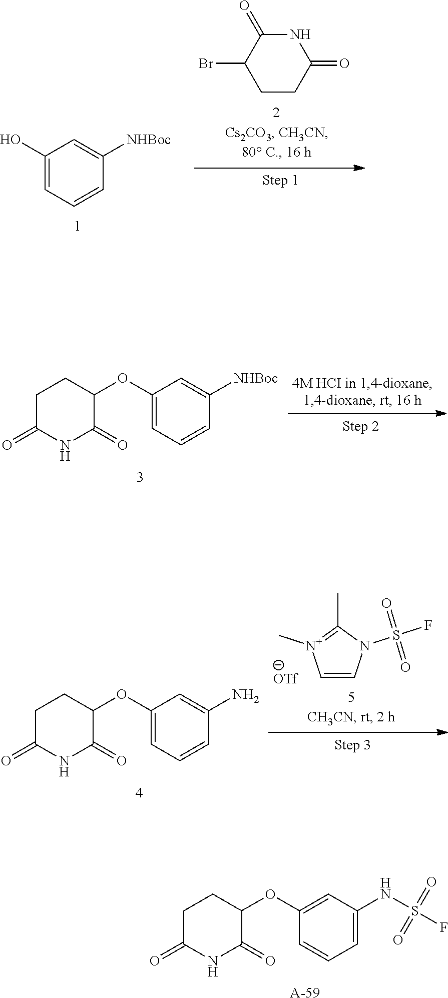

Step-1: To a stirred solution of tert-butyl N-(3-hydroxyphenyl) carbamate 1 (2.0 g, 9.56 mmol) in acetonitrile (40 mL) were added cesium carbonate (9.34 g, 28.7 mmol), 3-bromopiperidine-2,6-dione 2 (5.51 g, 28.68 mmol) and stirred at 80° C. for 16 hours. The reaction mixture was filtered through a celite pad and washed with acetonitrile (30 mL) and ethyl acetate (30 mL).

The filtrate was evaporated under reduced pressure to obtain the crude product. The crude product was purified by column chromatography (Davisil silica gel, using 50% ethyl acetate in 15 petroleum ether as an eluent) to afford tert-butyl N-[3-[(2,6-dioxo-3-piperidyl) oxy]phenyl]carbamate 3 (900 mg, 29% yield) as a white solid. LCMS (ES-): m z 319.26 [M-H]-.

Step-2: To a stirred solution of tert-butyl N-[3-[(2,6-dioxo-3-piperidyl) oxy]phenyl]carbamate 3 (600 mg, 1.87 mmol) in 1,4-dioxane (10 mL) was added dioxane in HCl (4 M, 10 mL) and 20 stirred at room temperature for 16 hours. The reaction mixture was evaporated under reduced pressure, diluted with a saturated aqueous sodium bicarbonate solution (15 mL) and extracted with ethyl acetate (3×15 mL). The combined organic layer was dried over anhydrous sodium sulfate and evaporated under reduced pressure to afford 3-(3-aminophenoxy) piperidine-2,6-dione 4 (300 mg, 59% yield) as an off-white solid. LCMS (ES*): m z 221.22 [M+H]+.

Step-3: To a stirred solution of 3-(3-aminophenoxy) piperidine-2,6-dione 4 (100 mg, 454.08 mol) in acetonitrile (5 mL) added 1-(fluorosulfonyl)-2,3-dimethyl-1H-imidazol-3-ium trifluoromethanesulfonate 5 (224.27 mg, 681.12 mol) and stirred at room temperature for 2 hours. The reaction mixture was diluted with water (5 mL) and extracted with ethyl acetate (2×5 mL). The combined organic layer was dried over anhydrous sodium sulfate and evaporated under reduced pressure to afford the crude product, which was purified by achiral SFC prep-purification [Column: YMC PAK DIOL-120, 30×250 mm, 5 micron, Mobile phase A: 68% CO₂, Mobile phase B: 32% acetonitrile: isopropyl Alcohol (1:1), Wavelength: 215 nm]and the fractions containing the compound were combined to afford N-[3-[(2,6-dioxo-3-piperidyl)oxy]phenyl]sulfamoyl fluoride (A-59) (25 mg, 17% yield) as an off-white solid.

LCMS (ES-): m z 301.07 [M-H]-. H-NMR (400 MHz, DMSO-d6): δ 11.0-11.1 (s, 1H), 7.36 (t, J=8.4 Hz, 1H), 6.99-6.36 (m, 1H), 6.88-6.86 (m, 2H), 5.25-5.22 (m, 1H), 2.76-2.5 (m, 2H), 2.23-2.07 (m, 2H).

Synthesis A57: Synthesis of 7-(2,6-dioxopiperidin-3-yl)naphthalene-2-sulfonyl fluoride (A-60)

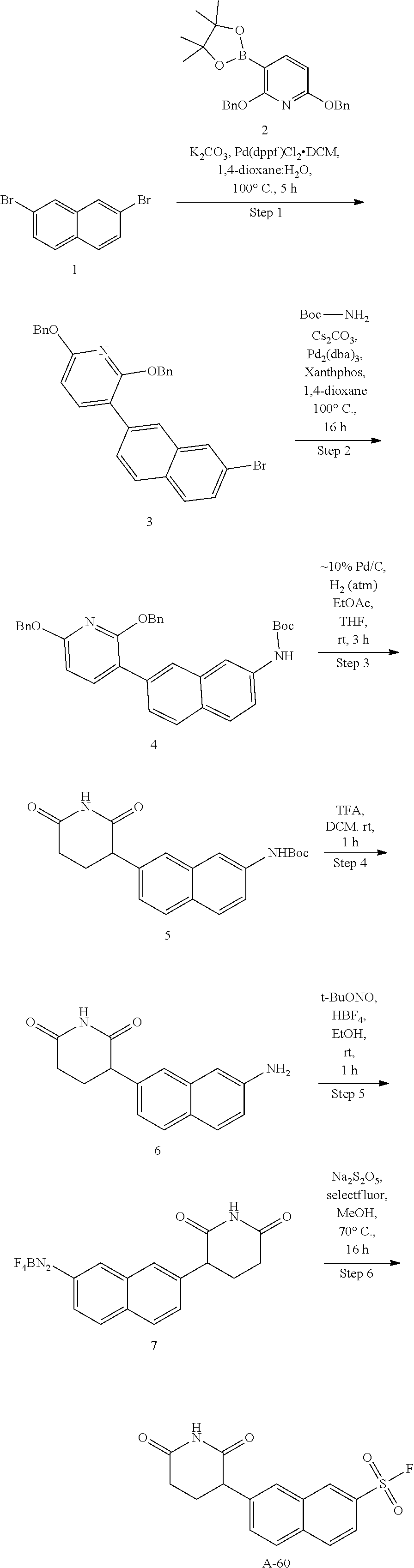

Step-1:

To a stirred solution of 2,7-dibromonaphthalene 1 (3 g, 10.49 mmol) and 2,6-dibenzyloxy-3-(4,4,5,5-tetramethyl-1,3,2-dioxaborolan-2-yl)pyridine 2 (3.06 g, 7.34 mmol) in 1,4-dioxane (30 mL) and water (2 mL) was added potassium phosphate (6.68 g, 31.5 mmol) and the reaction mixture was degassed with N2 gas for 10 minutes. Next, [1,1′-bis(diphenylphosphino)ferrocene]dichloropalladium(II) complex with dichloromethane (856.72 mg, 1.05 mmol) was added to the reaction mixture and the reaction mixture was 10 degassed with N2 for 5 minutes. The reaction mixture was stirred at 100° C. for 5 hours. Upon completion of the reaction, the reaction mixture was filtered through a celite bed, washed with ethyl acetate (20 mL). The filtrate was diluted with water (10 mL), extracted with ethyl acetate (2×20 mL), dried over anhydrous sodium sulfate and concentrated under reduced pressure to afford the crude product. The crude product was purified by column chromatography (Devisal silica gel, using 4% ethyl acetate in pet. ether as an eluent) to afford 2,6-dibenzyloxy-3-(7-bromo-2-naphthyl)pyridine 3 (1.2 g, 21% yield) as an off-white solid. LCMS (ES*): m z 498.41[M+H]+.

Step-2: To a stirred solution of 2,6-dibenzyloxy-3-(7-bromo-2-naphthyl)pyridine 3 (600 mg, 20 1.21 mmol) and tert-butyl carbamate (283.19 mg, 2.42 mmol) in 1,4-dioxane (10 mL) was added cesium carbonate (1.18 g, 3.63 mmol) and the reaction mixture was degassed with argon gas for 10 minutes. After, Pd2(dba)3 (110.68 mg, 120.87 mol) and Xanthphos (139.88 mg, 241.74 mol) were added, and the reaction mixture was degassed with argon gas for 2 min and stirred at 100° C. for 16 hours. Upon completion of the reaction, reaction mixture was filtered 25 through a celite bed, washed with ethyl acetate (20 mL) and the filtrate was concentrated under reduced pressure to afford the crude product which was purified by flash column chromatography (Daviscil silica gel, using 4% ethyl acetate in petroleum ether as an eluent) to afford tert-butyl N-[7-(2,6-dibenzyloxy-3-pyridyl)-2-naphthyl]carbamate 4 (0.5 g, 66% yield) as a pale-yellow solid. LCMS (ES+): m z 533.50 [M+H]+. 5 Step-3: To a stirred solution of tert-butyl N-[7-(2,6-dibenzyloxy-3-pyridyl)-2-naphthyl]carbamate 4 (0.5 g, 938.74 mol) in tetrahydrofuran (10 mL) and ethyl acetate (10 mL). were added palladium 10% on carbon (type 487, dry) (499.50 mg, 4.69 mmol) to the reaction mixture and stirred at room temperature for 3 hours under H2 atmosphere (balloon). Upon completion 10 of the reaction, the reaction mixture was filtered through celite bed and washed with ethyl acetate (20 mL), the filtrate was concentrated under reduced pressure to afford tert-butyl N-[7-(2,6-dioxo-3-piperidyl)-2-naphthyl]carbamate 5 (0.33 g, 92% yield) as an off-white solid.

LCMS (ES-): m z 353.32 [M-H]-.

Step-4: To a stirred solution of tert-butyl N-[7-(2,6-dioxo-3-piperidyl)-2-naphthyl]carbamate 5 (0.33 g, 931.15 mol) in dichloromethane (5.19 mL) was added trifluoroacetic acid (1.07 mL, 13.97 mmol) dropwise at 0° C. and stirred at room temperature for 1 hour. Upon completion of the reaction, the reaction mixture was concentrated under reduced pressure to afford the crude product. The crude product was triturated with n-pentane (1OmL) and concentrated under reduced pressure to afford 3-(7-amino-2-naphthyl) piperidine-2,6-dione 6 (0.34 g, 96% yield, trifluoroacetate salt) as a pale-yellow solid. LCMS (ES+): m z 255.32 [M+H]+.

Step-5: To a stirred solution of 3-(7-amino-2-naphthyl)piperidine-2,6-dione 6 (0.15 g, 589.89 mol) in ethanol (6 mL) was added trifluoroborane hydrofluoride (103.60 mg, 1.18 mmol) and tert-butyl nitrite (140.32 L, 1.18 mmol) at 0° C. and stirred at room temperature for 1 hour.

Upon completion of the reaction, the reaction mixture was cooled to 0° C. and diethyl ether (15 mL) was added and the reaction was stirred for 10 minutes, the diethyl ether layer (15 mL) was evaporated under reduced pressure to afford the crude product which was triturated with diethyl ether (10 mL) and dried under reduced pressure to afford 7-(2,6-dioxo-3-piperidyl)naphthalene-2-diazonium;tetrafluoroborate 7 (0.13 g, 37% yield) as a reddish-brown solid. Note: The crude was taken to next reaction without further purification. H-NMR (400 MHz, DMSO-d6): δ 11.03 (s, 1H), 8.39-8.46 (m, 2H), 8.25 (d, J=10.00 Hz, 2H), 7.92-8.04 (m, 2H), 4.23 (t, J=12.80 Hz, 1H), 2.35 (s, 2H), 2.15-2.18 (m, 2H).

Step-5: To a stirred solution of 7-(2,6-dioxo-3-piperidyl)naphthalene-2-diazonium;tetrafluoroborate (0.13 g, 368.19 mol) in anhydrous methanol (6 mL) was added sodium metabisulfite (209.99 mg, 1.10 mmol) and purged with N2 for 5 minutes. Next, 1-(chloromethyl)-4-fluoro-1,4-diazoniabicyclo[2.2.2]octane;ditetrafluoroborate (260.87 mg, 736.38 mol) was added to the reaction and the reaction mixture was stirred at 70° C. for 16 hours. The reaction mixture was filtered through a celite bed and washed with methanol (2×5 mL). The filtrate was concentrated under reduced pressure to afford the crude product. The crude product was purified by preparative HPLC [Column: X-BRIDGE C18, 19×250 mm, 5 10 micron, Mobile phase A: 0.1% trifluoroacetic acid in water, Mobile phase B: acetonitrile, Wavelength: 215 nm]and the fractions containing the compound were combined and lyophilized to afford the desired product 7-(2,6-dioxo-3-piperidyl)naphthalene-2-sulfonyl fluoride (A-60) (10.5 mg, 9% yield) as a pale-pink solid. LCMS (ES-): m z 320.04 [M-H]-. H-NMR (400 MHz, DMSO-d6): δ 10.96 (s, 1H), 8.91 (s, 1H), 8.31 (d, J=8.8 Hz, 1H), 8.17 (t, J=16.40 Hz, 2H), 8.04 (dd, J=10.40 Hz, 1H), 7.76 (d, J=8.80 Hz, 1H), 4.13-4.17 (m, 1H), 2.73-2.81 (m, 1H), 2.56-2.61 (m, 1H), 2.41 (d, J=4.40 Hz, 1H), 2.07-2.16 (m, 1H).

Synthesis A58: Synthesis of 5-((2,6-dioxopiperidin-3-yl)(methyl)amino)-2-(piperidin-4-yl)phenyl sulfurofluoridate (A-61) B0 BocN 3NO NO₂ BnBr, K₂CO₃ PdCl₂(dffp)DCM, K₂CO₃, KI,EtOH,80° C.,16 h 1,4-dioxane, water, 80° C.,2 h BnO OH Step 1 OBn Step 2 Br BrN Boc 1 2 4

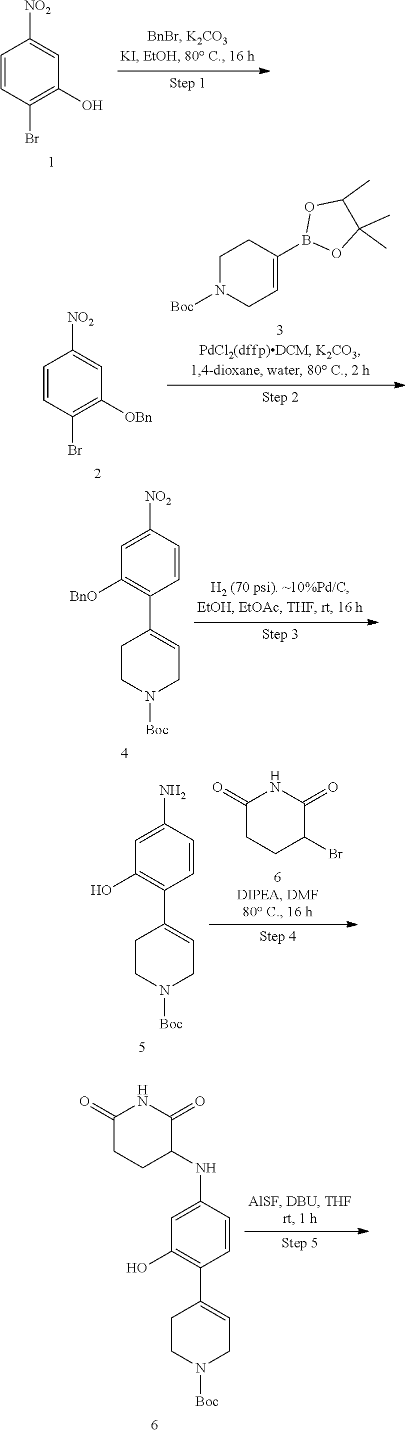

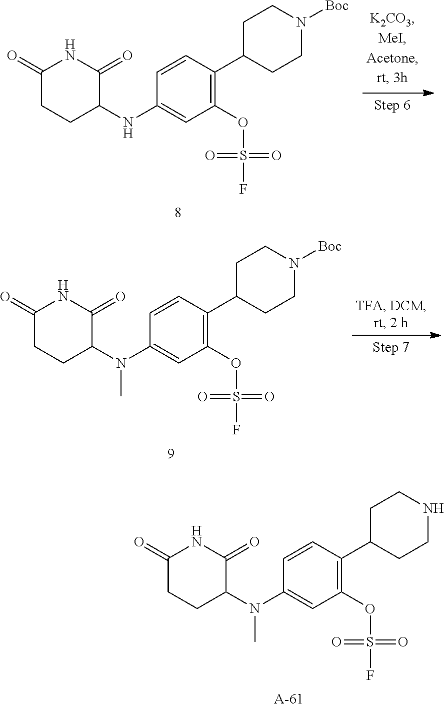 [SMILES: CC(C)(C)OC(=O)N1CCC(c2ccc(NC3CCC(=O)NC3=O)cc2OS(=O)(=O)F)CC1 ; CN(c1ccc(C2CCN(C(=O)OC(C)(C)C)CC2)c(OS(=O)(=O)F)c1)C1CCC(=O)NC1=O ; CN(c1ccc(C2CCNCC2)c(OS(=O)(=O)F)c1)C1CCC(=O)NC1=O ; "A-61"]

To a stirred solution of 2-bromo-5-nitro-phenol 1 (30 g, 137.61 mmol) in ethanol (100 mL) was added potassium carbonate (22.82 g, 165.13 mmol) and stirred at 90° C. for 2 hours.

Benzyl bromide (21.28 mL, 178.89 mmol) and potassium iodide (11.42 g, 68.81 mmol) were added to the reaction mixture and heated at 90° C. for 16 hours. Upon completion of the reaction, the reaction mixture evaporated under reduced pressure and the residue obtained was 10 diluted with water (3×300 mL) and extracted with ethyl acetate (3×350 mL). The combined organic layers were dried over sodium sulfate and concentrated under reduced pressure to afford the crude product. The crude product was purified by column chromatography (Davisil silica gel, using 10% ethyl acetate in petroleum ether as an eluent) to afford 2-benzyloxy-1-bromo-4-nitro-benzene 2 (41 g, 93% yield) as a pale-yellow solid. LCMS (ES-): m z 307.24 15 (M-H)-.

Step-2: To a stirred solution of 2-benzyloxy-1-bromo-4-nitro-benzene 2 (12 g, 38.94 mmol) and tert-butyl-4-(4,4,5,5-tetramethyl-1,3,2-dioxaborolan-2-yl)-3,6-dihydro-2H-pyridine-1-carboxylate 3 (13.25 g, 42.84 mmol) in 1,4-dioxane (85 mL) and water (15 mL) was added 5 potassium carbonate (16.15 g, 116.83 mmol) and degassed with argon gas for 10 minutes before adding Pd(dppf)C12 DCM (2.54 g, 3.12 mmol) and stirred at 80° C. for 2 hours. Upon completion of the reaction, the reaction mixture was filtered through a celite bed and washed with ethyl acetate (200 mL), and the filtrate was concentrated under reduced pressure to afford the crude product. The crude product was purified by column chromatography (Silica gel (230-400 mesh), using 30% ethyl acetate in petroleum ether as eluent) to afford tert-butyl 4-(2-benzyloxy-4-nitro-phenyl)-3,6-dihydro-2H-pyridine-1-carboxylate 4 (8.5 g, 45% yield) as a yellow color liquid. LCMS (ES+): m z 311.25 [M-Boc+H]+.

Step-3: To a stirred solution of tert-butyl 4-(2-benzyloxy-4-nitro-phenyl)-3, 6-dihydro-2H-pyridine-1-carboxylate 4 (13 g, 31.67 mmol) in tetrahydrofuran (30 mL), ethanol (30 mL) and ethyl acetate (40 mL) was added Pd/C (10% on carbon (wetted with ca. 55% Water) (3.37 g, 31.67 mmol) and stirred under H2 atmosphere (70 psi) at room temperature for 16 hours. Upon completion of the reaction, the reaction mixture was filtered through a celite bed and washed with dichloromethane (200 mL), the obtained filtrate was concentrated under reduced pressure to afford the crude product tert-butyl 4-(4-amino-2-hydroxy-phenyl)piperidine-1-carboxylate 5 (9.36 g, 45% yield) as a brown solid. LCMS (ES-): m z 291.22 (M-H)-.

Step-4: To a stirred solution of tert-butyl 4-(4-amino-2-hydroxy-phenyl) piperidine-1-carboxylate 5 (8 g, 27.36 mmol) in dimethylformamide (40 mL) was added N,N-diisopropylethylamine (14.30 mL, 82.09 mmol) and stirred for 10 minutes. After, 3-bromopiperidine-2,6-dione 6 (7.88 g, 41.04 mmol) was added to the reaction mixture and stirred at 80° C. for 16 hours. Upon completion of the reaction, the reaction was quenched with water (300 mL) and extracted with ethyl acetate (3×100 mL). The combined organic layer was washed with saturated sodium chloride solution (150 mL), dried over anhydrous sodium sulfate and concentrated under reduced pressure to afford the crude product. The crude product was purified by column chromatography using (Silica gel (60-120mesh), using 60% ethyl acetate in petroleum ether as eluent) to afford tert-butyl 4-[4-[(2,6-dioxo-3-piperidyl)amino]-2-hydroxy-phenyl]piperidine-1-carboxylate 7 (1.6 g, 11% yield) as a brown solid. LCMS (ES-): m z 402.37 [M-H]-.

Step-5: To a stirred solution of tert-butyl 4-[4-[(2,6-dioxo-3-piperidyl)amino]-2-hydroxy-phenyl]piperidine-1-carboxylate 7 (1.3 g, 3.22 mmol) in tetrahydrofuran (13 mL) was added DBU (1.20 mL, 8.06 mmol) and 4-acetamidobenzenesulfonyl azide (928.88 mg, 3.87 mmol) 5 at room temperature and stirred at room temperature for 1 hour. Upon completion of the reaction, the reaction mixture was concentrated under reduced pressure to afford the crude product. The crude was purified by column chromatography (silica gel (60-120 mesh), using 60% ethyl acetate in petroleum ether as an eluent) to afford tert-butyl 4-[4-[(2,6-dioxo-3-piperidyl)amino]-2-fluorosulfonyloxy-phenyl]piperidine-1-carboxylate 8 (0.53 g, 29% yield) as an off-white solid. LCMS (ES-): m z 430.34 [M-tBu+H]+. ¹H-NMR (400 MHz, DMSO-d6): 6 10.88 (s, 1H), 7.21 (d, J=9.2 Hz, 1H), 6.75 (d, J=6.4 Hz, 2H), 6.37 (d, J=8.0 Hz, 1H), 4.35-4.41 (m, 1H), 4.05-4.08 (m, 2H), 2.69-2.78 (m, 4H), 2.59-2.60 (m, 1H), 2.06-2.10 (m, 1H), 1.90-1.95 (m, 1H), 1.85-1.88 (m, 2H), 1.63 (s, 11H).

Step-6: A stirred solution of tert-butyl 4-[4-[(2,6-dioxo-3-piperidyl)amino]-2-fluorosulfonyloxy-phenyl]piperidine-1-carboxylate 8 (0.1 g, 205.96 mol) in methanol (2 mL) were added acetic acid (1.24 mg, 20.60 mol) and formaldehyde (~37% w/w aq. soln., stab. with 7-8% methanol) (8.57 pL, 308.94 mol) at 0° C. and stirred for 5 minutes. After, sodium cyanoboranuide (19.41 mg, 308.94 mol) was added to the reaction mixture at 0° C. and stirred 20 at room temperature for 16 hours. Upon completion of reaction, the reaction mixture was evaporated under reduced pressure to afford the crude product, the crude product was purified by preparative HPLC [Column: X-BRIDGE C18, 19×250 mm, 5 micron, Mobile phase A: 0.1% formic acid in water, Mobile phase B: acetonitrile, Wavelength: 215 nm]and the fractions containing the compound were combined and lyophilized to afford the desired product tert-butyl 4-[4-[(2,6-dioxo-3-piperidyl)-methyl-amino]-2-fluorosulfonyloxy-phenyl]piperidine-1-carboxylate 9 (17 mg, 16% yield) as an off-white solid. LCMS (ES-): m z 498.21 (M-H)-.

Step-7: To a stirred solution of tert-butyl 4-[4-[(2,6-dioxo-3-piperidyl)amino]-2-fluorosulfonyloxy-phenyl]piperidine-1-carboxylate 9 (17 mg, 35.01 mol) in dichloromethane (2 mL) was added trifluoroacetic acid (32.15 L, 420.16 mol) at 0° C. and stirred at room temperature for 2 hours. Upon completion of the reaction, the reaction mixture was concentrated under reduced pressure to afford the crude product. The crude product was triturated with diethyl ether (2×5 mL) and lyophilized to afford 3-[3-fluorosulfonyloxy-N-methyl-4-(4-piperidyl)anilino]-2,6-dioxo-piperidine (A-61) (12.74 mg, 71% yield) as an off-white solid. LCMS (ES*): m z 400.24 [M+H]+. H-NMR (400 MHz, DMSO-d6): δ 10.86 (s, 1H), 7.21 (d, J=8.80 Hz, 1H), 6.99 (dd, J=6.40 Hz, 1H), 6.89 (bs, 1H), 4.94 (m, 1H), 3.40-3.37 (m, 2H), 3.10-3.00 (m, 3H), 2.76 (s, 4H), 2.62-2.59 (m, 1H), 2.40-2.30 (m, 1H), 1.90-1.82 (m, 5H).

Synthesis A59: Synthesis of 3-(2,6-dioxopiperidin-3-yl)benzo[d]isoxazol-7-yl sulfurofluoridate (A-62)

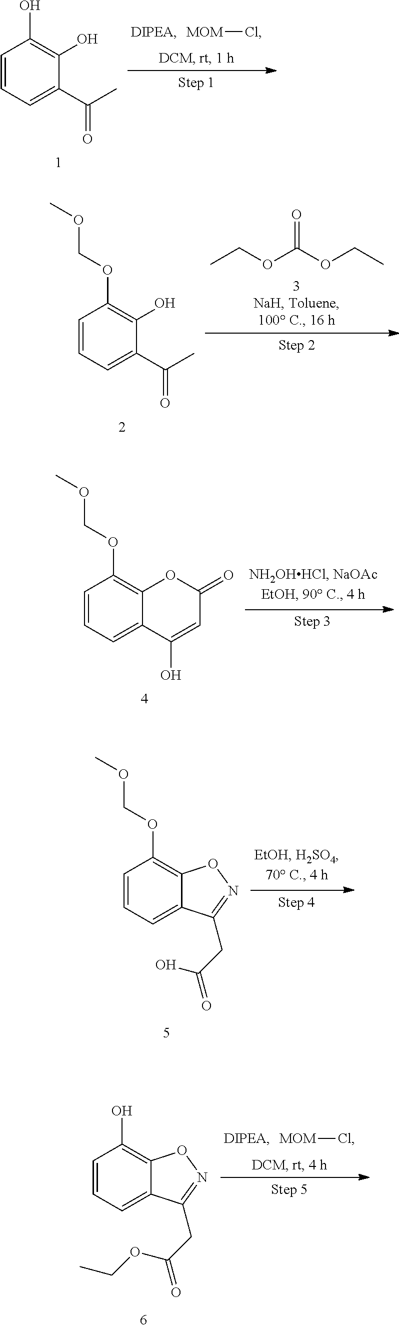

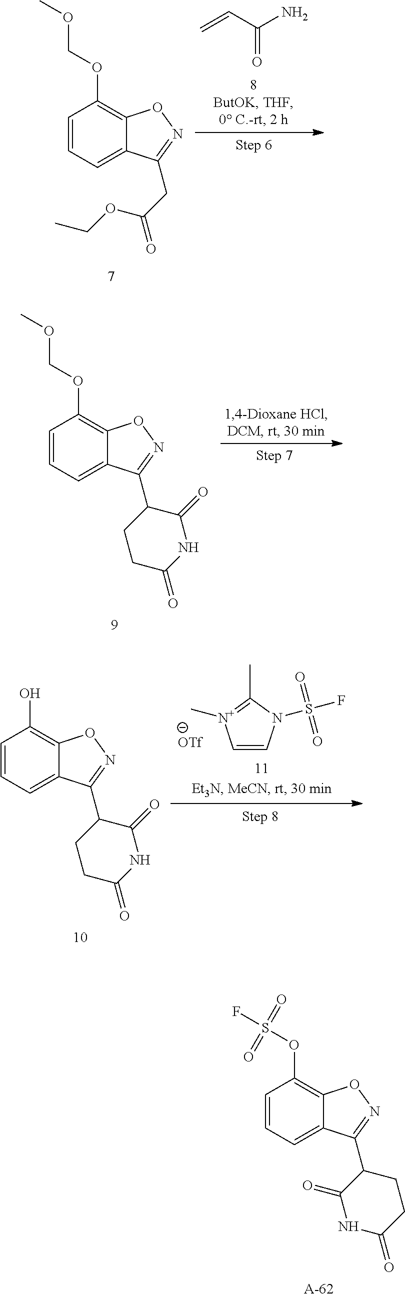

To an oven dried 100 mL round bottom flask charged with 1-(2,3-dihydroxyphenyl)ethanone 1 (2 g, 13.15 mmol) in dichloromethane (20 mL) were added diisopropylethylamine (4.58 mL, 26.29 mmol) and chloro(methoxy)methane (1.59 mL 19.72 mmol) at −10° C. The reaction mixture was stirred at room temperature for 1 hour. Upon completion of the reaction, the reaction mixture was diluted with water and extracted with ethyl acetate (3×20 mL). The organic layer was washed with saturated sodium chloride solution, dried over anhydrous sodium sulfate, filtered and concentrated under reduced pressure to afford 1-[2-hydroxy-3-(methoxymethoxy)phenyl]ethanone 2 (1.48 g, 52% yield) as a brown oil. (Note: The crude product taken to next reaction without further purification). H-NMR (400 MHz, DMSO-d6) δ: 12.12 (s, 1H), 7.57 (dd, J=9.20 Hz, 1H), 7.35 (dd, J=9.20 Hz, 1H), 6.89 (t, J=16.00 Hz, 1H), 5.20 (s, 2H), 2.33 (s, 3H), 2.65 (s, 3H).

Step-2: To an oven dried 100 mL round bottom flask charged with, a solution of 1-[2-hydroxy-3-(methoxymethoxy)phenyl]ethanone 2 (1.48 g, 7.54 mmol) in toluene (20 mL) was added NaH (60% dispersion in oil) (905.11 mg, 37.72 mmol) at 0° C. and stirred for 15 minutes.

Diethyl carbonate 3 (3.66 mL, 30.17 mmol) was added and the reaction mixture was stirred at 100° C. for 16 hours. Upon completion of the reaction, the reaction mixture was diluted 20 with water and extracted with ethyl acetate (3×50 mL). The aqueous layer was separated and acidified using 1.5 N HCl and the precipitated solid was filtered and dried under reduced pressure to afford 4-hydroxy-8-(methoxymethoxy)chromen-2-one 4 (1.3 g, 77% yield) as an off-white solid. LCMS (ES-): m z 221.18 [M-H]-.

Step-3: To an oven dried 100 mL round bottom flask charged with, a solution of 4-hydroxy-8-(methoxymethoxy)chromen-2-one 4 (0.3 g, 1.35 mmol) in ethanol (5 mL) were added sodium acetate (0.217 mL, 4.05 mmol) and hydroxylamine hydrochloride (281.47 mg, 4.05 mmol) and the reaction mixture was stirred at 90° C. for 4 hours. Upon completion of the reaction, the reaction mixture was diluted with water and extracted with ethyl acetate (3×20 mL). The combined organic layer was washed with saturated sodium chloride solution, dried over anhydrous sodium sulfate, filtered and evaporated under reduced pressure to afford the 5 crude product, which was purified by combi flash column chromatography (Davisil silica, using 40-45% ethyl acetate in petroleum ether as an eluent) to afford 2-[7-(methoxymethoxy)-1,2-benzoxazol-3-yl]acetic acid 5 (0.1 g, 30% yield) as a brown solid. LCMS (ES*): m z 238.22 [M+H]+.

Step-4: To an oven dried 25 mL round bottom flask charged with a solution of 2-[7-(methoxymethoxy)-1,2-benzoxazol-3-yl]acetic acid 5 (100 mg, 0.421 mmol) in ethanol (5 mL) was added 4 M solution of sulfuric acid in water (0.210 mL) and stirred at 90° C. for 4 h. Upon completion of the reaction, the reaction mixture was basified using saturated sodium bicarbonate solution and extracted with ethyl acetate (3×30 mL). The combined organic layer 15 was washed with saturated sodium chloride solution, dried over anhydrous sodium sulfate, and evaporated under reduced pressure to afford ethyl 2-(7-hydroxy-1,2-benzoxazol-3-yl)acetate 6 (0.1 g, 76% yield) as a brown oil. LCMS (ES-): m z 220.22 [M-H]-.

Step-5: To an oven dried 25 mL round bottom flask charged with, a solution ofethyl 2-(7-hydroxy-1,2-benzoxazol-3-yl)acetate 6 (0.1 g, 0.452 mmol) in dichloromethane (5 mL) were added diisopropylethylamine (0.157 mL, 0.904 mmol) and chloro(methoxy)methane (0.054 mL 0.678 mmol) at 0° C. and stirred at room temperature for 2 hours. Upon completion of the reaction, the reaction mixture was diluted with water and extracted with dichloromethane (3×20 mL). The organic layer was washed with saturated sodium chloride solution, dried over anhydrous sodium sulfate, filtered and concentrated under reduced pressure to afford ethyl 2-[7-(methoxymethoxy)-1,2-benzoxazol-3-yl]acetate 7 (0.1 g, 37% yield) as a brown liquid. LC-MS (ES+): m z 266.16 [M+H]+.

Step-6: To a stirred solution ofethyl 2-[7-(methoxymethoxy)-1,2-benzoxazol-3-yl]acetate 7 (0.1 g, 0.376 mmol) and prop-2-enamide 8 (53.59 mg, 0.753 mmol) intetrahydrofuran (3 mL) was added KOBu (50.76 mg, 0.452 mmol) at 0° C. The resulting mixture was stirred at room temperature for 30 minutes. Upon completion of the reaction, the reaction mixture was diluted with water and extracted with ethyl acetate (3×20 mL). The combined organic layer was washed with saturated sodium chloride solution, dried over anhydrous sodium sulfate, filtered and evaporated under reduced pressure to afford the crude product which was purified by column chromatography (Davisil silica, using 45% ethyl acetate in petroleum ether as an eluent) to afford 3-[7-(methoxymethoxy)-1,2-benzoxazol-3-yl]piperidine-2,6-dione 9 (0.035 g, 29% yield) as a brownoil. LCMS (ES-): m z 289.12 [M-H]-.

Step-7: To an oven dried 10 mL round bottom flask charged with, a solution of 3-[7-(methoxymethoxy)-1,2-benzoxazol-3-yl]piperidine-2,6-dione 9 (0.03 g, 0.10 mmol) in dichloromethane (2 mL) was added 4 M HCl in 1,4-dioxane (0.129 mL, 0.51 mmol) at 0° C. and stirred at room temperature for 30 minutes. Upon completion of the reaction, the reaction mixture was evaporated under reduced pressure to obtain crude product which was triturated with diethyl ether and dioxane (4:1 ratio)to afford 3-(7-hydroxy-1,2-benzoxazol-3-yl)piperidine-2,6-dione 10 (0.025 g, 59% yield) as an off-white solid. LCMS (ES-): m z 245.18 [M-H]-.

Step-8: To an oven dried 10 mL round bottom flask charged with, a solution of 3-(7-hydroxy-1,2-benzoxazol-3-yl)piperidine-2,6-dione 10 (0.1 g, 0.406 mmol) and 2,3-dimethylimidazol-3-ium-1-sulfonyl fluoride 11 (266.64 mg, 0.812 mmol) in acetonitrile (2.0 mL) was added trimethylamine (0.169 mL, 1.22 mmol) at 0° C. and stirred at room temperature for 30 minutes. Upon completion of the reaction, the reaction mixture was diluted with water (5 mL) and extracted with ethyl acetate (3×5 mL). The combined organic layer was washed with saturated sodium chloride solution, dried over anhydrous sodium sulfate, filtered and evaporated under reduced pressure to obtain crude product, which was further purified by was purified by preparative HPLC [Column: X select C18, 10×250 mm, 5 micron, Mobile phase A: 0.1% formic acid in water, Mobile phase B: acetonitrile, Wavelength: 215 nm]and the fractions containing the compound were combined and lyophilized to afford the desired product 3-(2,6-doxopiperidin-3-yl)benzo[d]isoxazol-7-yl sulfurofluoridate (A-62) (5.8 mg, 5% yield) as an off-white solid. LCMS (ES-): m z 327.07 [M-H]-. H-NMR (400 MHz, DMSO-d6): 6 11.15 (s, 1H), 8.08-8.03 (m, 2H), 7.61-7.57 (m, 1H), 4.76-4.71 (m, 1H), 2.83-2.74 (m, 1H), 2.63-2.49 (m, 2H), 2.28-2.23 (m, 1H).

Synthesis A60: Synthesis of 2-(2,6-dioxopiperidin-3-yl)—N-isopropyl-3-oxoisoindoline-5-sulfonimidoyl fluoride (A-63)

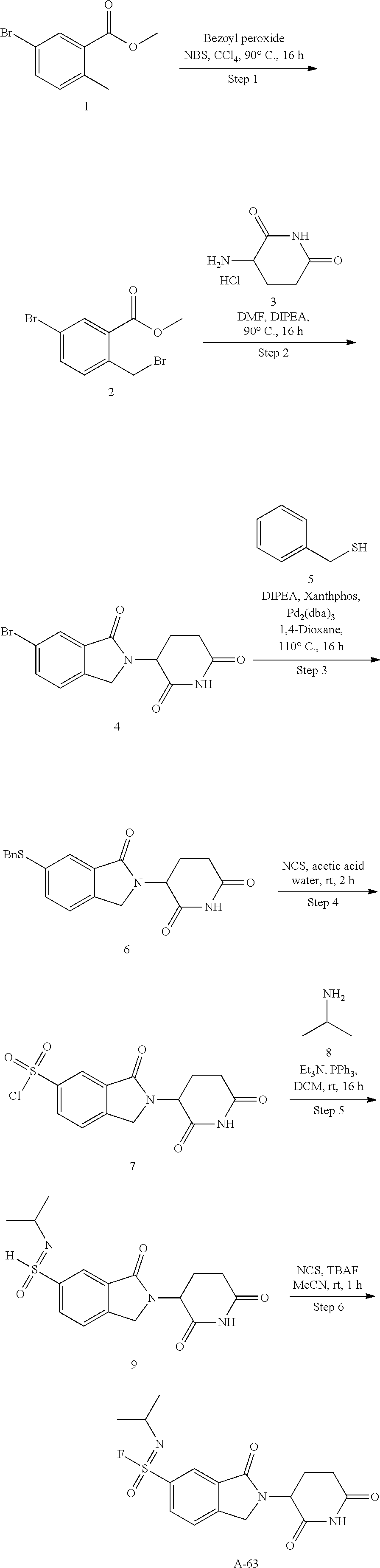

Step-1: To an oven dried 100 mL round bottom flask charged with methyl 5-bromo-2-methyl-benzoate 1 (5 g, 21.83 mmol) in carbon tetrachloride (100 mL) were added N-bromosuccinimide (4.27 g, 24.01 mmol) and azobisisobutyronitrile (1.79 g, 10.91 mmol) at 10 room temperature and heated at 90° C. for 16 hours. Upon completion of the reaction, the reaction mixture was diluted with water (100 mL) and extracted with dichloromethane (3×100 mL). The organic layer was dried over anhydrous sodium sulfate, filtered and evaporated under reduced pressure to obtain crude product which was purified by column chromatography (Davisil silica, using 1% ethyl acetate in petroleum ether as an eluent) to afford methyl 5-bromo-2-(bromomethyl) benzoate 2 (3.80 g, 51% yield) as yellow oil. H-NMR (400 MHz, CDCl3) 6: 8.11-8.10 (m, 1H), 7.62 (dd, J=8.2, 2.2 Hz, 1H), 7.34 (d, J=8.0 Hz, 1H), 4.90 (s, 2H), 3.95 (s, 3H).

Step-2: To an oven dried 100 mL seal tube charged with methyl 5-bromo-2-(bromomethyl) benzoate 2 (3.50 g, 11.36 mmol), 3-aminopiperidine-2,6-dione 3 (2.18 g, 13.27 mmol, HCl salt) in dimethylformamide (25 mL) was added N,N-diisopropylethylamine (9.90 mL, 56.82 mmol) and heated at 90° C. for 16 hours. Upon completion of the reaction, ice cold water (200 mL) was added to the reaction mixture, the precipitate obtained was filtered and dried to afford 3-(6-bromo-1-oxo-isoindolin-2-yl) piperidine-2,6-dione 4 (2.20 g, 53% yield) as a violet solid.

LCMS (ES-): m z 321.14 [M-H]-.

Step-3: To an oven dried 100 mL seal tube charged with a solution of 3-(6-bromo-1-oxo-isoindolin-2-yl) piperidine-2,6-dione 4 (2.0 g, 6.19 mmol), phenylmethanethiol 5 (1.54 g, 12.38 mmol) in 1,4-dioxane (30 mL) was added N,N-diisopropylethylamine (3.23 mL, 18.57 mmol) and degassed with N2 (atm) for 5 minutes before adding Pd2(dba)3 (566.76 mg, 0.61 mmol) and Xanthphos (716.24 mg, 1.24 mmol). Reaction mixture was stirred at 110° C. for 16 hours.

Upon completion of the reaction, the reaction mixture was filtered on a pad of celite and washed with ethyl acetate (50 mL) and dichloromethane (100 mL). The filtrate was evaporated under reduced pressure to obtain crude product which was triturated with n-pentane (50 mL) to afford 3-(6-benzylsulfanyl-1-oxo-isoindolin-2-yl) piperidine-2,6-dione 6 (2.0 g, 85% yield). LC-MS (ES*): m/z 367.19 [M+H]+.

Step-4: To an oven dried 25 mL round bottom flask charged with a solution of 3-(6-benzylsulfanyl-1-oxo-isoindolin-2-yl) piperidine-2,6-dione 6 (2.0 g, 5.46 mmol) in acetic acid (32 mL), water (16 mL) was added N-chlorosuccinimide (273.31 mg, 2.05 mmol) at room temperature and stirred for 2 hours. Upon completion of the reaction, the precipitate obtained in the reaction mixture was filtered and washed with diethyl ether (50 mL) to afford 2-(2,6-dioxo-3-piperidyl)-3-oxo-isoindoline-5-sulfonyl chloride 7 (1.0 g, 51% yield) as an off-white solid. ¹H-NMR (400 MHz, DMSO-d6): δ 10.93 (s, 1H), 7.83-7.79 (m, 2H), 7.51 (d, J=8.0 Hz, 1H), 5.05 (dd, J=13.2, 5.2 Hz, 1H), 4.41 (d, J=17.6 Hz, 1H), 4.28 (d, J=17.6 Hz, 1H), 2.89-2.80 (m, 1H), 2.56-2.51 (m, 1H), 2.36-2.29 (m, 1H), 2.01-1.98 (m, 1H).

Step-5: To an oven dried 25 mL round bottom flask charged with a solution of 2-(2,6-dioxo-3-piperidyl)-3-oxo-isoindoline-5-sulfonyl chloride 7 (500 mg, 1.46 mmol) in dichloromethane (8 mL) were added a drop wise solution of isopropylamine (0.12 mL, 1.46 mmol,), triphenylphosphine (382.61 mg, 1.46 mmol), triethylamine (0.40 mL, 2.92 mmol) in dichloromethane (2 mL) at 0° C. The reaction mixture was stirred at room temperature for 16 hours. Upon completion of the reaction, reaction mixture was diluted with water (20 mL) and extracted using dichloromethane (2×20 mL). The organic layer was dried over anhydrous sodium sulfate, filtered and evaporated under reduced pressure to obtain crude product which was purified by column chromatography (Davisil silica, using as an ethyl acetate as eluent) to afford 3-[6-[isopropylimino(oxo)-sulfanyl]-1-oxo-isoindolin-2-yl]piperidine-2,6-dione 8 (200 mg, 37% yield) as a white solid. LCMS (ES*): m z 350.38 [M+H]+.

Step-6: To an oven dried 25 mL round bottom flask charged with, a solution of 3-[6-[isopropylimino(oxo)-sulfanyl]-1-oxo-isoindolin-2-yl]piperidine-2,6-dione 8 (100 mg, 0.28 mmol) in acetonitrile (5 mL) was added TBAF in tetrahydrofuran (1 M, 0.31 mL, 0.31 mmol) and stirred at room temperature for 5 minutes. N-chlorosuccinimide (42 mg, 0.31 mmol) was added at 0° C. portion-wise and stirred at room temperature for 1 hour. Upon completion of the 10 reaction, reaction mixture was diluted with water (20 mL) and extracted with ethyl acetate (2×25 mL), the combined organic layer was dried over anhydrous sodium sulfate, filtered and evaporated under reduced pressure to obtain crude product. The crude product was purified by prep-SFC achiral [Column: ethyl pyridine, 30×250 mm, 5 micron, Mobile phase A: C02: 80%, Mobile phase B: 20% (acetonitrile: isopropanol) (1:1), Wavelength: 215 nm]and the fractions containing the compound were concentrated to afford 3-[6-(S-fluoro-N-isopropyl-sulfonimidoyl)-1-oxo-isoindolin-2-yl]piperidine-2,6-dione (A-63) (26.4 mg, 25% yield) as white solid. LCMS (ES-): m z 366.09 [M-H]-. H-NMR (400 MHz, CD30D) 6: 8.43 (d, J=1.6 Hz, 1H), 8.29 (dd, J=8.2, 1.8 Hz, 1H), 7.86 (d, J=8.0 Hz, 1H), 5.22-5.17 (m, 1H), 4.70-4.59 (m, 2H), 4.11-4.07 (m, 1H), 2.93-2.87 (m, 1H), 2.82-2.80 (m, 1H), 2.53-2.49 (m, 1H), 2.23-2.18 (m, 1H), 1.36-1.31 (m, 6H).

Synthesis A61: Synthesis of 7-((1-(4-aminobutyl)-1H-1,2,3-triazol-4-yl)methoxy)-2-(2,6-dioxopiperidin-3-yl)-3-oxoisoindoline-5-sulfonyl fluoride (A-64):

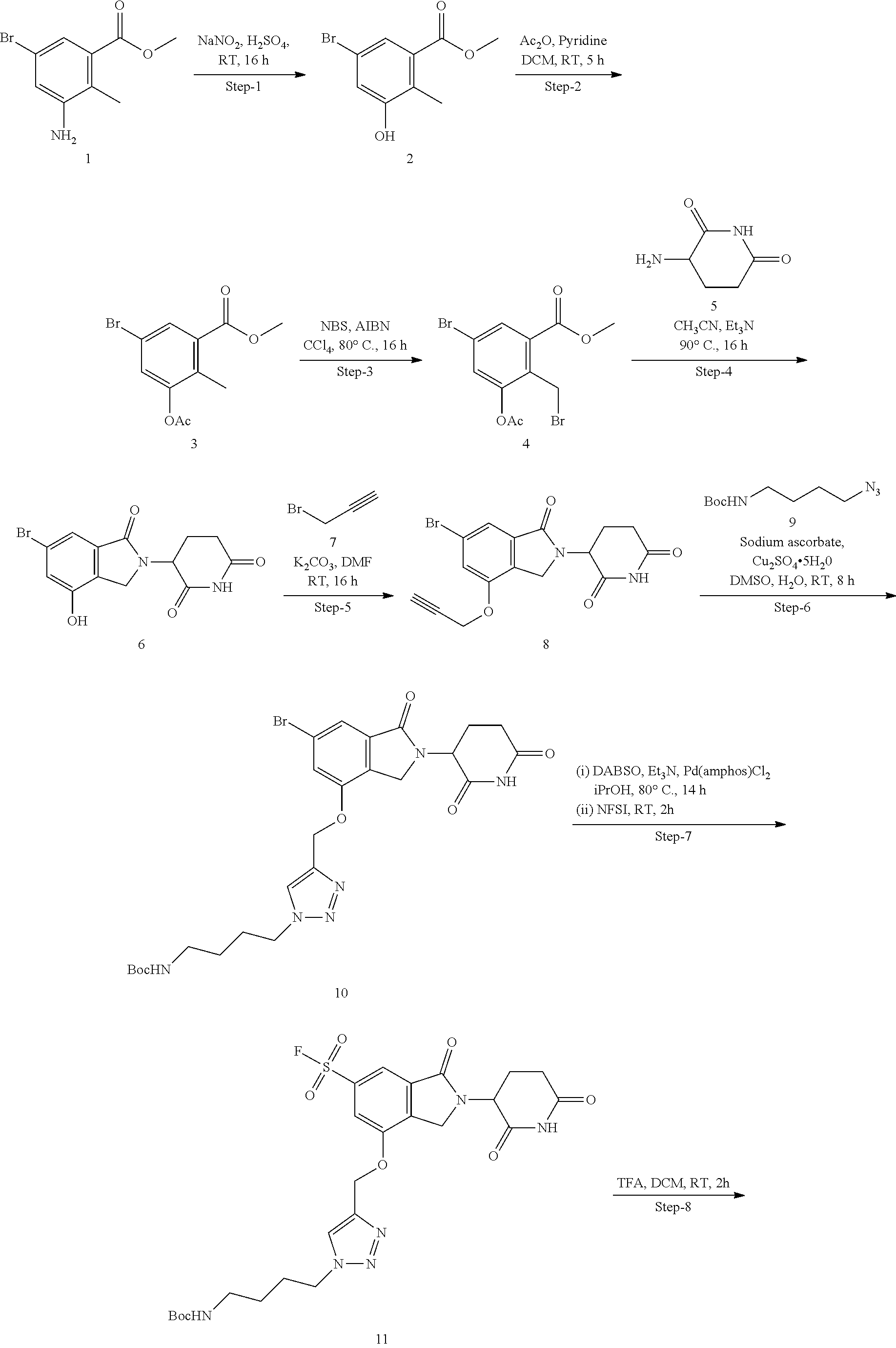

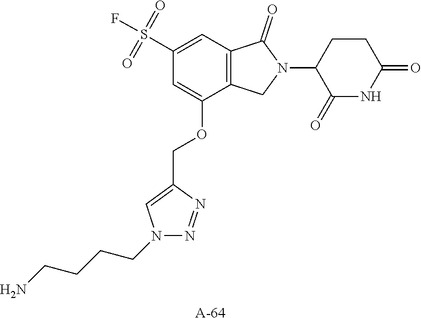 [SMILES: NCCCCn1cc(COc2cc(S(=O)(=O)F)cc3c2CN(C2CCC(=O)NC2=O)C3=O)nn1 ; "A-64"]

To a stirred solution of methyl 3-amino-5-bromo-2-methyl-benzoate 1 (20 g, 81.94 mmol) in concentrated sulfuric acid (60 mL) was added NaNO2 (6.8 g, 98.56 mmol) in portions at 0° C. The resulting mixture was stirred for 16 hours at room temperature. After completion, the resulting mixture was diluted with water (200 mL) and extracted with ethyl acetate (300 mL×3). The combined organic layer was washed with saturated sodium chloride solution (100 10 mL) and dried over sodium sulfate, filtered and concentrated under reduced pressure to afford methyl 5-bromo-3-hydroxy-2-methyl-benzoate 2 (13.5 g, 64% yield) as a light-yellow solid.

LCMS (ES-): m z 243.05 [M-H]-.

Step-2: To a solution of methyl 5-bromo-3-hydroxy-2-methyl-benzoate 2 (10 g, 40.80 mmol) 15 in dichloromethane (50 mL) were added pyridine (6.57 mL, 81.61 mmol), acetic anhydride (7.70 mL, 81.61 mmol) and stirred at room temperature for 3 hours. After completion of the reaction, the reaction mixture was concentrated under reduced pressure to obtain crude compound, which was diluted with water (100 mL) and extracted with ethyl acetate (200 mL×3). The combined organic layer was dried over sodium sulfate and concentrated under reduced pressure to afford methyl 3-acetoxy-5-bromo-2-methyl-benzoate 3 (11.0 g, 93% yield) as a light-yellow solid. LCMS (ES+): m z 287.13 [M+H]+.

Step-3: To a stirred solution of methyl 3-acetoxy-5-bromo-2-methyl-benzoate 3 (6 g, 20.90 mmol) in carbon tetrachloride (75 mL) was added N-bromosuccinimide (4.46 g, 25.08 mmol) at room temperature and stirred the reaction mixture at 80° C. for 16 hours. After completion, the reaction mixture was filtered through celite bed and washed with CCl₄ (30 mL). The filtrate was concentrated under reduced pressure and triturated with pentane (50 mL) to afford methyl 3-acetoxy-5-bromo-2-(bromomethyl) benzoate 4 (6.65 g, 78% yield) as a light-yellow solid. H-NMR (400 MHz, DMSO-d6): δ 7.99 (d, J=2.0 Hz, 1H), 7.49 (d, J=2.0 Hz, 1H), 4.87 (s, 2H), 3.95 (s, 3H), 2.41 (s, 3H.

Step-4: To a stirred solution of methyl 3-acetoxy-5-bromo-2-(bromomethyl)benzoate 4 (6.65 g, 18.17 mmol), 3-aminopiperidine-2,6-dione 5 (3.89 g, 23.62 mmol, hydrochloric acid) in acetonitrile (35 mL) was added triethylamine (7.60 mL, 54.51 mmol) and stirred at 90° C. for 16 hours. After completion, the reaction mixture was concentrated under reduced pressure to obtain the crude product. The crude product was suspended in (1:1) ratio of ethyl acetate and water (50 mL). The precipitate was collected and triturated with diethyl ether (50 mL) and dried under reduced pressure to afford 3-(6-bromo-4-hydroxy-1-oxo-isoindolin-2-yl)piperidine-2,6-dione 6 (2.5 g, 37% yield) as an purple color solid. LCMS (ES-): m z 339.15 [M-H]-.

Step-5: To a stirred solution of 3-(6-bromo-4-hydroxy-1-oxo-isoindolin-2-yl)piperidine-2,6-dione 6 (2.5 g, 7.37 mmol) in dimethylformamide (15 mL) was added potassium carbonate (2.04 g, 14.74 mmol) and propargyl bromide 7 (670.26 pL, 8.85 mmol). The reaction mixture was stirred at room temperature for 16 hours. After completion, the reaction mixture was diluted with water (30 mL) and extracted with ethyl acetate (100 mL×2). The combined organic layer was dried over sodium sulfate and concentrated under reduced pressure to obtained crude product, which was purified by flash column chromatography (Davisil silica) using 70% of ethyl acetate in petroleum ether as eluent to afford 3-(6-bromo-1-oxo-4-prop-2-ynoxy-isoindolin-2-yl)piperidine-2,6-dione 8 (1.1 g, 29% yield) as an off-white solid. LCMS (ES+): m z 379.29 [M+H]+Step-6: To a stirred solution of 3-(6-bromo-1-oxo-4-prop-2-ynoxy-isoindolin-2-yl)piperidine-2,6-dione 8 (1.1 g, 2.92 mmol) in dimethyl sulfoxide (10 mL), water (10 mL) were added copper sulfate pentahydrate (728.16 mg, 2.92 mmol), sodium ascorbate (577.74 mg, 2.92 mmol) and tert-butyl N-(4-azidobutyl)carbamate 9 (624.86 mg, 2.92 mmol). The reaction mixture was stirred at room temperature for 8 hours. After completion, the reaction mixture was diluted with water (30 mL) and extracted with ethyl acetate (100 mL×2). The combined organic layer was dried over sodium sulfate, filtered and concentrated under reduced pressure to obtained crude product, which was purified by flash column chromatography (Davisil silica) using 70% of ethyl acetate in petroleum ether as eluent to afford 3-(6-bromo-1-oxo-4-prop-2-ynoxy-isoindolin-2-yl)piperidine-2,6-dione 10 (1.1 g, 29% yield) as an off-white solid. H-NMR (400 MHz, DMSO-d6): δ 10.98 (s, 1H), 8.25 (s, 1H), 7.68 (s, 1H), 6.83 (t, J=5.2 Hz, 1H), 5.33 (s, 2H), 5.11-5.06 (m, 1H), 4.38-4.30 (m, 3H), 4.03 (t, J=7.2 Hz, 1H), 2.94-2.86 (m, 4H), 2.60-2.55 (m, 2H), 2.42-2.38 (m, 1H), 1.99-1.96 (m, 1H), 1.85-1.71 (m, 3H), 1.45-1.35 (m, 9H).

Step-7: A glass reaction tube was charged with DABCO(SO2)2 (48.75 mg, 0.202 mmol), Pd(amphos)C12 (23.94 mg, 0.033 mmol) and tert-butyl N-[4-[4-[[6-bromo-2-(2,6-dioxo-3-piperidyl)-1-oxo-isoindolin-4-yl]oxymethyl]triazol-1-yl]butyl]carbamate 10 (0.2 g, 0.338 mmol) sealed with a rubber septum and degassing with N2 for 5 minutes. Anhydrous isopropanol (10 mL) and anhydrous trimethylamine (141.39 L, 1.01 mmol) were added sequentially through the septum and the reaction mixture stirred under positive pressure of N2 in a preheated oil bath at 80° C. for 14 hours. After cooling to room temperature, N-fluorobenzenesulfonimide (159.95 mg, 0.507 mmol) was added and the reaction mixture stirred for 2 hours until completion. The reaction mixture was concentrated under reduced pressure to obtain crude product, which was diluted with saturated aqueous Na2S203 solution (10 mL) and extracted with ethyl acetate (30 mL×2). The combined organic layer was washed with saturated sodium chloride solution (50 mL), dried over anhydrous sodium sulfate, filtered and concentrated under reduced pressure to afford crude product. The crude product was purified by preparative HPLC [Column: UNISIL, 21×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product tert-butyl N-[4-[4-[[2-(2,6-dioxo-3-piperidyl)-6-fluorosulfonyl-1-oxo-isoindolin-4-yl]oxymethyl]triazol-1-yl]butyl]carbamate 11 (50 mg, 25% yield) as an off-white solid. LCMS (ES-): m z 593.41[M-H]-Step 8: To a stirred solution of tert-butyl N-[4-[4-[[2-(2,6-dioxo-3-piperidyl)-6-fluorosulfonyl-1-oxo-isoindolin-4-yl]oxymethyl]triazol-1-yl]butyl]carbamate 11 (50 mg, 0.084 mmol) in dichloromethane (5 mL) was added trifluoroacetic acid (128.70 L, 1.68 mmol) at 0 C The reaction mixture was warmed to room temperature and continued the stirring for 2 hours. After completion of the reaction, the reaction mixture was concentrated under reduced pressure to obtain crude product, which was purified by preparative HPLC [Column: X-BRIDGE C18, 19 10×250 mm, 5 micron, Mobile phase A: 0.1% trifluoroacetic acid in water, Mobile phase B: acetonitrile, Wavelength: 215 nm]and the fractions containing the compound were combined and lyophilized to afford the desired product 7-[[1-(4-aminobutyl)triazol-4-yl]methoxy]-2-(2,6-dioxo-3-piperidyl)-3-oxo-isoindoline-5-sulfonyl fluoride (A-64) (28.3 mg, 54% yield, trifluoroacetic acid) as an off-white solid. LCMS (ES*): m z 495.29 [M+H]+. H-NMR (400 MHz, DMSO-d6): δ 11.02 (s, 1H), 8.29 (s, 1H), 8.15 (d, J=1.2 Hz, 1H), 7.95 (s, 1H), 7.62 (brs, 2H), 5.52 (s, 2H), 5.18-5.14 (m, 1H), 4.59-4.31 (m, 4H), 2.95-2.84 (m, 1H), 2.81 (t, J=7.6 Hz, 2H), 2.63-2.59 (m, 1H), 2.43-2.41 (m, 1H), 2.08-1.95 (m, 1H), 1.91-1.80 (m, 2H), 1.59-1.40 (m, 2H) ppm.

Synthesis A62: Synthesis of 7-(2-((4-aminobutyl)amino)-2-oxoethoxy)-2-(2,6-dioxopiperidin-3-yl)-3-oxoisoindoline-5-sulfonyl fluoride (A-65):

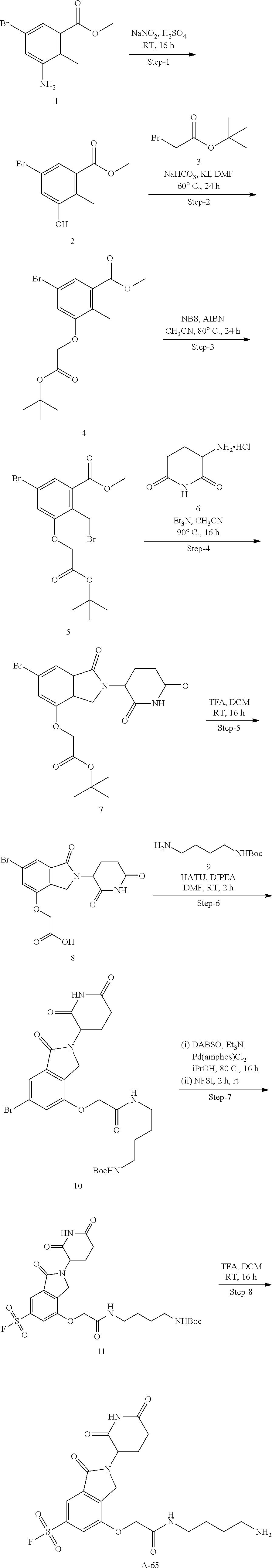

Step-1:

To a stirred solution of methyl 3-amino-5-bromo-2-methyl-benzoate 1 (30 g, 122.91 mmol) in concentrated sulfuric acid (65 mL) was added sodium nitrite (10.18 g, 147.49 mmol) in portions at 0° C. The resulting mixture was stirred for 16 hours at room temperature. After completion of the reaction, the resulting mixture was diluted with water (200 mL) and extracted with ethyl acetate (300 mL×3). The combined organic layer was washed with saturated sodium 10 chloride solution (300 mL) and dried over sodium sulfate, filtered and concentrated under reduced pressure to afford crude product, which was purified by flash column chromatography (Davisil silica using 10% ethyl acetate in petroleum ether as an eluent) to afford methyl 5-bromo-3-hydroxy-2-methyl-benzoate 2 (16.5 g, 52% yield) as a light-yellow solid. LCMS (ES-): m z 245.08 [M-H]-.

Step-2: To a stirred solution of methyl 5-bromo-3-hydroxy-2-methyl-benzoate 2 (7.5 g, 30.60 mmol) in dimethylformamide (80 mL) were added KI (2.54 g, 15.30 mmol), sodium bicarbonate (5.14 g, 61.21 mmol) followed by tert-butyl 2-bromoacetate 3 (8.98 mL, 61.21 mmol) at room temperature. The reaction mixture was stirred at 60° C. for 24 hours. After completion of the reaction, the reaction mixture was diluted with water (100 mL) and extracted with ethyl acetate (3×200 mL). The combined organic layer was washed with saturated sodium chloride solution (100 mL), dried over sodium sulfate and concentrated under reduced pressure to afford methyl 5-bromo-3-(2-tert-butoxy-2-oxo-ethoxy)-2-methyl-benzoate 4 (6.0 g, 47% yield) as light-yellow solid. LCMS (ES*): m z 382.45 [M+Na]f.

Step-3: To a stirred solution of methyl 5-bromo-3-(2-tert-butoxy-2-oxo-ethoxy)-2-methyl-benzoate 4 (6 g, 16.70 mmol) in acetonitrile (60 mL) was added N-bromosuccinimide (3.57 g, 20.04 mmol) at room temperature. The reaction mixture was stirred at 80° C. for 24 hours. After completion of reaction, the reaction mixture was filtered through celite bed and washed with carbon tetrachloride (50 mL). The obtained filtrate was concentrated under reduced pressure to afford methyl 5-bromo-2-(bromomethyl)-3-(2-tert-butoxy-2-oxo-ethoxy) benzoate 5 (8 g, 66% yield) as brown color oil. H-NMR (400 MHz, DMSO-d6): δ 7.59 (d, J=2.0 Hz, 1H), 7.28 (d, J=2.0 Hz, 1H), 4.94 (s, 2H), 4.82 (s, 2H), 3.88 (s, 3H), 1.42 (s, 9H). Step-4: To a stirred solution of methyl 5-bromo-2-(bromomethyl)-3-(2-tert-butoxy-2-oxo-ethoxy) benzoate 5 (8.0 g, 18.26 mmol), 3-aminopiperidine-2,6-dione 6 (4.51 g, 27.39 mmol, hydrochloric acid) in acetonitrile (50 mL) was added triethylamine (7.64 mL, 54.78 mmol) and stirred at 90° C. for 16 hours. After completion of the reaction, the reaction mixture was concentrated under reduced pressure to obtain crude residue. The crude residue was diluted with water (10 mL) and extracted with ethyl acetate (20 mL×2). The combined organic layer was washed with saturated sodium chloride solution (20 mL), dried over anhydrous sodium sulfate, filtered and concentrated under reduced pressure to afford tert-butyl 2-[6-bromo-2-(2,6-dioxo-3-piperidyl)-1-oxo-isoindolin-4-yl]oxyacetate 7 (4 g, 36% yield) as purple solid. LCMS (ES+): m z 397.30 [M-^{t}Bu+H]+Step-5: To a stirred solution of tert-butyl 2-((6-bromo-2-(2,6-dioxopiperidin-3-yl)-1-oxoisoindolin-4-yl) oxy) acetate 7 (0.45 g, 0.992 mmol) in dichloromethane (15 mL) was added trifluoroacetic acid (1.52 mL, 19.86 mmol) at 0° C. The reaction mixture was warm to room temperature and continued the stirring for 16 hours. After completion, the reaction mixture was concentrated under reduced pressure to obtain crude product, which was triturated with diethyl ether (3×15 mL) to afford 2-[6-bromo-2-(2,6-dioxo-3-piperidyl)-1-oxo-isoindolin-4-yl]oxyacetic acid 8 (0.35 g, 62% yield, trifluoroacetic acid) as an off-white solid. LCMS (ES*): m z 397.26 [M+H]+.

Step-6: To a solution of 2-[6-bromo-2-(2,6-dioxo-3-piperidyl)-1-oxo-isoindolin-4-yl]oxyacetic acid 8 (0.35 g, 0.881 mmol) and tert-butyl N-(4-aminobutyl) carbamate 9 (182.49 mg, 0.969 mmol) in dimethylformamide (6 mL) was added N,N-diisopropylethylamine (0.767 mL, 4.41 mmol) followed by HATU (502.60 mg, 1.32 mmol) and stirred at room temperature for 2 hours. After completion of the reaction, the reaction mixture was quenched with ice-cold water (5 mL), the precipitate so obtained was filtered and dried under reduced pressure to get the tert-butyl N-[4-[[2-[6-bromo-2-(2,6-dioxo-3-piperidyl)-1-oxo-isoindolin-4-yl]oxyacetyl]amino]butyl]carbamate 10 (0.3 g, 35% yield) as yellow solid. LCMS (ES+): m z 569.38 [M+H]+.

Step-7: A glass reaction tube was charged with DABCO(SO2)2 (76.23 mg, 0.317 mmol), Pd(amphos)C12 (37.44 mg, 0.052 mmol) and tert-butyl N-[4-[[2-[6-bromo-2-(2,6-dioxo-3-piperidyl)-1-oxo-isoindolin-4-yl]oxyacetyl]amino]butyl]carbamate 10 (0.3 g, 0.528 mmol) sealed with a rubber septum and evacuated and filled back with N2 four times. Anhydrous isopropanol (5 mL) and anhydrous triethylamine (221.07 L, 1.59 mmol) were added sequentially through the septum and the reaction mixture stirred under positive pressure of N2 in a preheated oil bath at 80° C. for 16 hours. After cooling to room temperature N-fluorobenzenesulfonimide (250.08 mg, 0.793 mmol) was added, and stirred for 2 hours. The reaction mixture was concentrated under reduced pressure to obtain crude product, which was diluted with saturated aqueous Na2S203 solution (10 mL) and extracted with ethyl acetate (30 mL×2). The combined organic layer was washed with saturated sodium chloride solution (50 mL), dried over anhydrous sodium sulfate, filtered and concentrated under reduced pressure to afford tert-butyl N-[4-[[2-[2-(2,6-dioxo-3-piperidyl)-6-fluorosulfonyl-1-oxo-isoindolin-4-yl]oxyacetyl]amino]butyl]carbamate 11 (0.4 g, 28% yield) as yellow solid. LCMS (ES-): m z 569.46 [M-H]-.

Step 8: To a stirred solution of tert-butyl N-[4-[[2-[2-(2,6-dioxo-3-piperidyl)-6-fluorosulfonyl-1-oxo-isoindolin-4-yl]oxyacetyl]amino]butyl]carbamate 11 (0.15 g, 0.262 mmol) in dichloromethane (5 mL) was added trifluoroacetic acid (20.12 L, 0.262 mmol) at 0° C. and stirred at room temperature for 2 hours. After completion of the reaction, the reaction mixture was concentrated under reduced pressure to get crude product, which was purified by preparative HPLC [Column: X-SELECT C18, 19×150 mm, 5 micron, Mobile phase A: 0.1% 5 trifluoroacetic acid in water, Mobile phase B: acetonitrile, Wavelength: 215 nm]and the fractions containing the compound were combined and lyophilized to afford the desired product 7-[2-(4-aminobutylamino)-2-oxo-ethoxy]-2-(2,6-dioxo-3-piperidyl)-3-oxo-isoindoline-5-sulfonyl fluoride (A-65) (22.8 mg, 14% yield, trifluoroacetic acid) as an off-white solid. ¹H-NMR (400 MHz, DMSO-d6): δ 11.05 (s, 1H), 8.25 (t, J=5.6 Hz, 1H), 7.97 (s, 1H), 7.77 (d, J=1.2 Hz, 1H), 7.72 (brs, 1H), 5.22-5.17 (m, 1H), 4.92-4.84 (m, 2H), 4.66-4.50 (m, 2H), 3.17-3.14 (m, 2H), 2.98-2.89 (m, 1H), 2.78 (t, J=6.8 Hz, 2H), 2.69-2.59 (m, 1H), 2.43-2.41 (m, 1H), 2.07-2.03 (m, 1H), 1.91 (s, 1H), 1.60-1.40 (m, 4H). LCMS (ES+): m z 471.27 [M+H]+.

Synthesis A63: Synthesis of 2-((2-(2,6-dioxopiperidin-3-yl)-6-(fluorosulfonyl)-1-oxoisoindolin-4-yl)oxy)acetic acid (A-66):

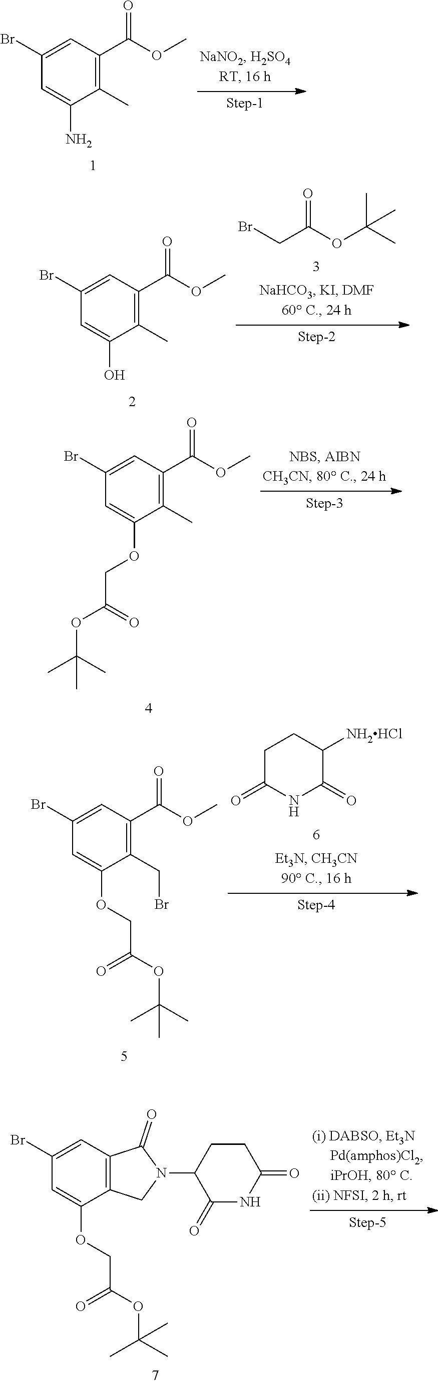

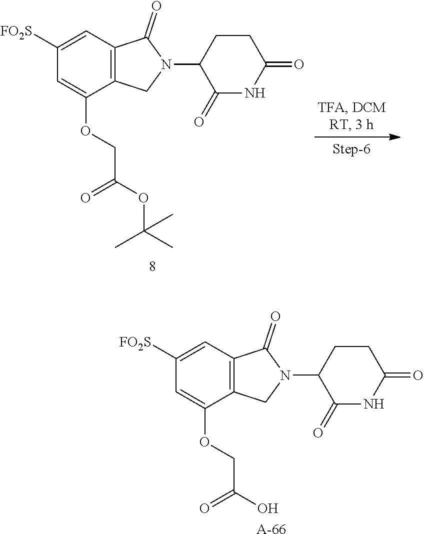

Step-1:

To a stirred solution of methyl 3-amino-5-bromo-2-methyl-benzoate 1 (30 g, 122.91 mmol) in concentrated sulfuric acid (65 mL) was added sodium nitrite (10.18 g, 147.49 mmol) 5 in portions at 0° C. The resulting mixture was stirred for 16 hours at room temperature. After completion of the reaction, the resulting mixture was diluted with water (200 mL) and extracted with ethyl acetate (300 mL×3). The combined organic layer was washed with saturated sodium chloride solution (300 mL) and dried over sodium sulfate, filtered and concentrated under reduced pressure to afford crude product, which was purified by flash column chromatography (Davisil silica) using 10% ethyl acetate in petroleum ether to afford methyl 5-bromo-3-hydroxy-2-methyl-benzoate 2 (16.5 g, 52% yield) as a light-yellow solid. LCMS (ES-): m z 245.08 [M-H]-.

Step-2: To a stirred solution of methyl 5-bromo-3-hydroxy-2-methyl-benzoate 2 (7.5 g, 30.60 15 mmol) in dimethylformamide (80 mL) were added KI (2.54 g, 15.30 mmol), sodium bicarbonate (5.14 g, 61.21 mmol) followed by tert-butyl 2-bromoacetate 3 (8.98 mL, 61.21 mmol) at room temperature. The reaction mixture was stirred at 60° C. for 24 hours. After completion of the reaction, the reaction mixture was diluted with water (100 mL) and extracted with ethyl acetate (3×200 mL). The combined organic layer was washed with saturated sodium 20 chloride solution (100 mL), dried over sodium sulfate and concentrated under reduced pressure to afford methyl 5-bromo-3-(2-tert-butoxy-2-oxo-ethoxy)-2-methyl-benzoate 4 (6.0 g, 47% yield) as a light-yellow solid. LCMS (ES): m z 382.45 [M+Na]+.

Step-3: To a stirred solution of methyl 5-bromo-3-(2-tert-butoxy-2-oxo-ethoxy)-2-methyl-benzoate 4 (6 g, 16.70 mmol) in acetonitrile (60 mL) was added N-bromosuccinimide (3.57 g, 20.04 mmol) at room temperature. The reaction mixture was stirred at 80° C. for 24 hours. After completion of reaction, the reaction mixture was filtered through celite bed and washed with carbon tetrachloride (50 mL). The obtained filtrate was concentrated under reduced pressure to afford methyl 5-bromo-2-(bromomethyl)-3-(2-tert-butoxy-2-oxo-ethoxy) benzoate 5 (8 g, 66% yield) as brown oil. H-NMR (400 MHz, DMSO-d6): δ 7.59 (d, J=2.0 Hz, 1H), 7.28 (d, J=2.0 Hz, 1H), 4.94 (s, 2H), 4.82 (s, 2H), 3.88 (s, 3H), 1.42 (s, 9H).

Step-4: To a stirred solution of methyl 5-bromo-2-(bromomethyl)-3-(2-tert-butoxy-2-oxo-ethoxy) benzoate 5 (8.0 g, 18.26 mmol), 3-aminopiperidine-2,6-dione 6 (4.51 g, 27.39 mmol, hydrochloric acid) in acetonitrile (50 mL) was added triethylamine (7.64 mL, 54.78 mmol) and stirred at 90° C. for 16 hours. After completion of the reaction, the reaction mixture was concentrated under reduced pressure to obtain crude residue. The crude residue was diluted with water (10 mL) and extracted with ethyl acetate (20 mL×2). The combined organic layer 10 was washed with saturated sodium chloride solution (20 mL), dried over anhydrous sodium sulfate, filtered and concentrated under reduced pressure to afford tert-butyl 2-[6-bromo-2-(2,6-dioxo-3-piperidyl)-1-oxo-isoindolin-4-yl]oxyacetate 7 (4 g, 36% yield) as a purple solid.

LCMS (ES*): m z 397.30 [M-^{t}Bu+H]+.

Step-5: To a stirred solution of tert-butyl 2-[6-bromo-2-(2,6-dioxo-3-piperidyl)-1-oxo-isoindolin-4-yl]oxyacetate 7 (0.5 g, 1.10 mmol) in isopropanol (9.64 mL) were added triethylamine (461 L, 3.31 mmol), DABCO(SO2)2 (159 mg, 0.661 mmol) and purged with nitrogen gas for 10 minutes before adding Pd(amphos)C12 (78 mg, 0.110 mmol) at room temperature. The reaction mixture was stirred at 80° C. for 14 hours in sealed tube. Later the 20 reaction mixture was cooled to room temperature and added N-Fluorobenzenesulfonimide (522 mg, 1.65 mmol) and continued stirring at room temperature for 2 hours. After completion of reaction, the reaction mixture was diluted with water (30 mL) and extracted with ethyl acetate (2×30 mL). The combined organic layer was washed with saturated sodium chloride solution (30 mL), dried over anhydrous sodium sulfate and concentrated under reduced pressure to afford crude product tert-butyl 2-[2-(2,6-dioxo-3-piperidyl)-6-fluorosulfonyl-1-oxo-isoindolin-4-yl]oxyacetate 8 (0.6 g, 46% yield) as a yellow solid, which was used directly in the next step without further purification. LC-MS (ES-): m/z 455.31 [M-H]-.

Step-6: To a stirred solution of tert-butyl 2-[2-(2,6-dioxo-3-piperidyl)-6-fluorosulfonyl-1-oxo-isoindolin-4-yl]oxyacetate 8 (0.1 g, 0.219 mmol) in dichloromethane (5 mL) was added trifluoroacetic acid (335 L, 4.38 mmol) at 0° C. and stirred at room temperature for 3 hours.

After completion, the reaction mixture was concentrated under reduced pressure to obtain crude product, which was purified by preparative HPLC [Column: SUNFIRE C18, 19×150 mm, 5 micron, Mobile phase A: 0.1% trifluoroacetic acid in water, Mobile phase B: acetonitrile, Wavelength: 215 nm](25 mg) followed by SFC purification to afford 2-[2-(2,6-dioxo-3-piperidyl)-6-fluorosulfonyl-1-oxo-isoindolin-4-yl]oxyacetic acid (A-66) (11.1 mg, 12% yield) as an off-white solid. LCMS (ES*): m z 401.26 [M+H]+. H-NMR (400 MHz, DMSO-d6): δ 13.38 (br s, 1H), 11.02 (s, 1H), 7.91 (s, 1H), 7.78 (s, 1H), 5.18-5.13 (m, 1H), 4.95 (s, 2H), 4.61-4.42 (m, 2H), 2.96-2.87 (m, 1H), 2.62-2.57 (m, 1H), 2.46-2.43 (m, 1H), 2.09-2.02 (m, 1H). ¹⁹F NMR (376 MHz, DMSO-d6): δ 66.376.

General Procedure: One Pot Sonogashira Coupling followed by Click Cycloaddition

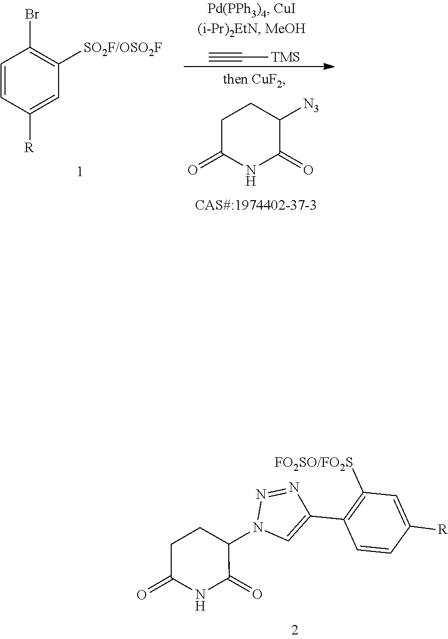

To a flask of ethynyltrimethylsilane (1.3 eq) at room temperature are added 1 (1 eq), Pd(PPh3)4 (0.05 equiv), CuI (0.10 equiv), (i-Pr)2EtN (2.0 equiv) and followed by MeOH. The reaction is stirred at 120° C. under microwave irradiation until judged complete. The reaction mixture is cooled to room temperature and CuF2(2.0 equiv) and 3-azidopiperidine-2,6-dione (CAS #:1974402-37-3, 1.0 equiv) are added and the reaction is stirred at 120° C. under 15 microwave irradiation until judged complete. The reaction is concentrated under reduced pressure and is purified under standard conditions to provide product 2.

Synthesis A64: Synthesis of 5-amino-2-(1-(2,6-dioxopiperidin-3-yl)-1H-1,2,3-triazol-4-yl)phenyl sulfurofluoridate (A-67): General Procedure: Br Br One pot Sonogashira FO₂SO OH AISF OSO₂F Coupling followed by N=N DBU, TH Click Cycloaddition, N NH₂ Step 1 Step 2 NH₂ NH₂ 0 N H hA S #- 551?0-56-4 A-67 P-69,C

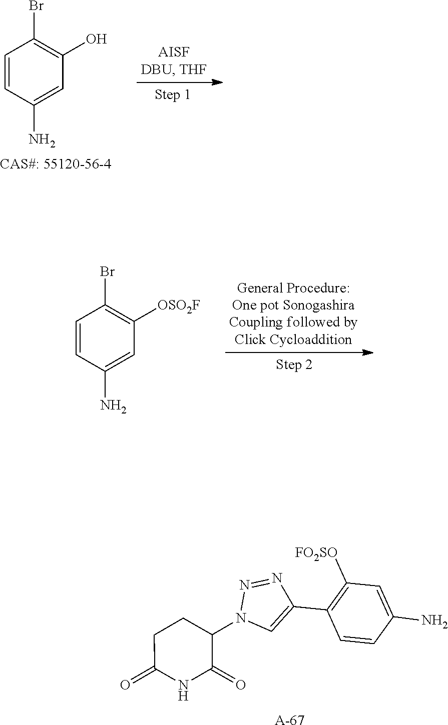

Step 1: 5-amino-2-bromophenyl sulfurofluoridate To a solution of 5-amino-2-bromophenol (CAS #: 55120-56-4, 1 eq) in THF at room temperature is added DBU (2 eq) followed by AISF (1.5 eq) and the reaction stirred until judged complete. A standard workup and purification provide 5-amino-2-bromophenyl sulflurofluoridate.

Step 2: 5-amino-2-(1-(2,6-dioxopiperidin-3-yl)-1H-1,2,3-triazol-4-yl)phenyl sulfurofluoridate (A-67) 5-amino-2-(1-(2,6-dioxopiperidin-3-yl)-1H-1,2,3-triazol-4-yl)phenyl sulfurofluoridate A-67 is prepared using General Procedure: One pot Sonogashira Coupling followed by Click Cycloaddition by using 5-amino-2-bromophenyl sulfurofluoridate as the bromide partner.

Synthesis A65: Synthesis of 5-bromo-2-(1-(2,6-dioxopiperidin-3-yl)-1H-1,2,3-triazol-4-yl)phenyl sulfurofluoridate (A-68):

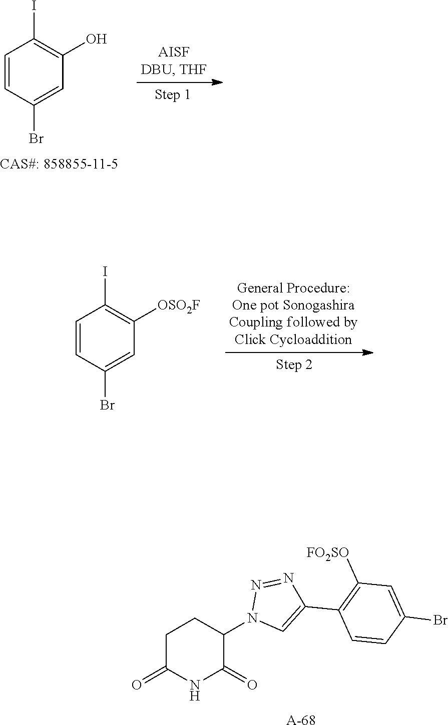

General Procedure: AISF I One pot Sonogashira FO₂SO OH OSO₂F Coupling followed by N=N DBU, THE Click Cycloaddition N/Br Step 1 Step 2 Br Br O N O H CA-S #- 858855-11-5 A-68

Step 1: 5-bromo-2-iodophenyl sulfurofluoridate To a solution of 5-bromo-2-iodophenol (CAS #: 858855-11-5, 1 eq) in THF at room temperature is added DBU (2 eq) followed by AISF (1.5 eq) and stirred until judged complete.

A standard workup and purification provide 5-bromo-2-iodophenyl sulfurofluoridate.

Step 2: 5-amino-2-(1-(2,6-dioxopiperidin-3-yl)-1H-1,2,3-triazol-4-yl)phenyl sulfurofluoridate (A-68) 5-bromo-2-(1-(2,6-dioxopiperidin-3-yl)-1H-1,2,3-triazol-4-yl)phenylsulfurofluoridate (A-68) is prepared using General Procedure: One pot Sonogashira Coupling followed by Click Cycloaddition and using 5-bromo-2-iodophenyl sulfurofluoridate as the bromide partner.

Synthesis A66: Synthesis of 2-(1-(2,6-dioxopiperidin-3-yl)-1H-1,2,3-triazol-4-yl)benzenesulfonyl fluoride (A-69):

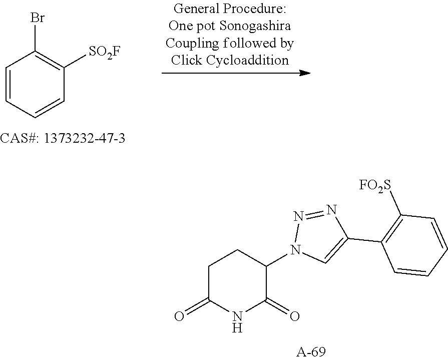

2-(1-(2,6-dioxopiperidin-3-yl)-1H-1,2,3-triazol-4-yl)benzenesulfonyl fluoride (A-69) is prepared using General Procedure: One pot Sonogashira Coupling followed by Click Cycloaddition and using 2-bromobenzenesulfonyl fluoride (CAS #:1373232-47-3) as the bromide partner.

Synthesis A67: Synthesis of 5-amino-2-(1-(2,6-dioxopiperidin-3-yl)-1H-1,2,3-triazol-4-yl)benzenesulfonyl fluoride (A-70):

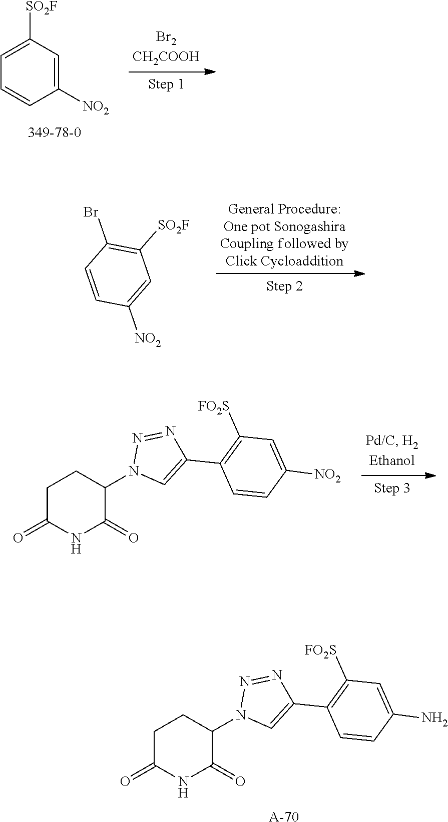

Step 1: 2-bromo-5-nitrobenzenesulfonyl fluoride To a solution of 3-nitrobenzenesulfonyl fluoride (CAS #: 349-78-0, 1 eq) in acetic acid 10 at room temperature is added bromine (1.2 eq) and stirred at 75° C. until judged complete. The reaction is quenched with an aqueous saturated sodium thiosulfate solution at 0° C. A standard workup and purification provide 2-bromo-5-nitrobenzenesulfonyl fluoride.

Step 2: 2-(1-(2,6-dioxopiperidin-3-yl)-1H-1,2,3-triazol-4-yl)-5-nitrobenzenesulfonyl fluoride 5-amino-2-(1-(2,6-dioxopiperidin-3-yl)-1H-1,2,3-triazol-4-yl)phenyl sulfurofluoridate is prepared using General Procedure: One pot Sonogashira Coupling followed by Click Cycloaddition using 2-bromo-5-nitrobenzenesulfonyl fluoride as the bromide partner.

Step 3: 5-amino-2-(1-(2,6-dioxopiperidin-3-yl)-1H-1,2,3-triazol-4-yl)benzenesulfonyl fluoride (A-70) To a flask is added 2-(1-(2,6-dioxopiperidin-3-yl)-1H-1,2,3-triazol-4-yl)-5-nitrobenzenesulfonyl fluoride (1 eq), in ethanol at room temperature is added 10% palladium on carbon (3 eq), and the reaction mixture is stirred at room temperature under a H2 atmosphere until the reaction is judged complete. The reaction is filtered through diatornaceous earth, concentrated under reduced pressure and subjected to standard purification to provide 5-amino-2-(1-(2,6-dioxopiperidin-3-yl)-1H-1,2,3-triazol-4-yl)benzenesulfonyl fluoride (A-70). Synthesis A68: Synthesis of 3-(1-(2,6-dioxopiperidin-3-yl)-1H-pyrazol-3-yl)phenyl sulfurofluoridate (A-71):

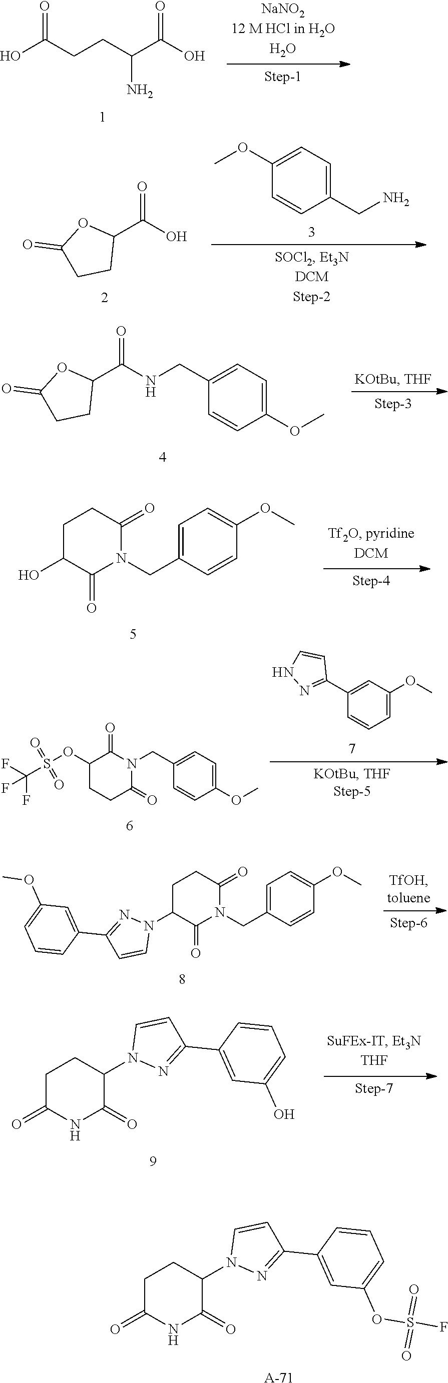

Step-1: To a solution of 2-aminopentanedioic acid (204 g, 1.39 mol) in water (870.97 10 mL) and 12 M HCl, 37% (1.39 mol, 204 mL) was added a solution of NaNO2 (143.51 g, 2.08 mol, 66.19 mL) in water (435.49 mL) at - 5° C. The mixture was stirred at 25° C. for 16 h. Upon completion of the reaction, the mixture was concentrated in vacuo and the residue was triturated with ethyl acetate, filtered and the obtained solid was dried under vacuum to afford 5-oxotetrahydrofuran-2-carboxylic acid (180 g, 1.25 mol, 89.81% yield) as yellow oil. ¹H NMR (400 MHz, CDCl3): δ 6.43 (s, 1H), 5.02-4.95 (m, 1H), 2.67-2.38 (m, 4H) ppm.

Step-2: To a round-bottom flask containing 5-oxotetrahydrofuran-2-carboxylic acid (100 g, 768.65 mmol) was added thionyl chloride (914.46 g, 7.69 mol, 560.68 mL) at 0° C. The mixture was slowly heated to 80° C. and stirred at this temperature for 4 h. Then, thinyl chloride 20 was removed under vacuum to furnish the corresponding acid chloride. To the acid chloride was added DCM (500 mL) and the solution was stirred at 0° C. for 15 min, before a solution of triethylamine (155.56 g, 1.54 mol, 214.27 mL) and 4-methoxybenzylamine (126.53 g, 922.38 mmol, 120.51 mL) in DCM (500 mL) was added at 0° C. The resulting mixture was stirred at room temperature for 16 h. After completion of the reaction, the reaction mixture was quenched with water and extracted with ethyl acetate. The separated organic layer was subsequently washed with iN HCl and concentrated in vacuo. The crude product was purified using column chromatography (80% EtOAc in Pet Ether) to afford N-[(4-methoxyphenyl)methyl]-5-oxo-tetrahydrofuran-2-carboxamide (114 g, 365.88 mmol, 47.60% yield) as a yellow solid.

Step-3: A solution of N-[(4-methoxyphenyl)methyl]-5-oxo-tetrahydrofuran-2-carboxamide (25 g, 100.30 mmol) in anhydrous THF (200 mL) was cooled to −78° C. before a solution of potassium tert-butoxide (14.63 g, 130.38 mmol) in anhydrous THF (175 mL) was added dropwise at −78° C. under nitrogen atmosphere. The resulting reaction mixture was stirred at -76° C. for 1 h. Upon completion of the reaction, the reaction mixture was quenched with saturated NH4C1 solution (310 mL) and the mixture was extracted with ethyl acetate (3×310 mL). The combined organic layer was washed with brine (500 mL), dried over anhydrous sodium sulfate, filtered and the filtrate was concentrated in vacuo. The residue was recrystallized in a mixture of DCM and Pet. Ether (1:9) to afford 3-hydroxy-1-[(4-methoxyphenyl)methyl]piperidine-2,6-dione (20 g, 64.19 mmol, 64.00% yield) as a pale brown solid.

Step-4: To a stirred solution of 3-hydroxy-1-[(4-methoxyphenyl)methyl]piperidine-2,6-dione (5 g, 20.06 mmol) in DCM (22.49 mL) were added pyridine (3.23 mL, 40.12 mmol) and trifluoromethanesulfonic anhydride (5.06 mL, 30.09 mmol) at 0° C. and the mixture was stirred at the same temperature for 1 h. The progress of reaction was monitored by TLC. After completion of the reaction, the reaction mixture was concentrated under reduced pressure and the obtained residue was purified by column chromatography (100-200 mesh silica gel) using a gradient of 20% ethyl acetate in pet ether to afford [1-[(4-methoxyphenyl)methyl]-2,6-dioxo-3-piperidyl]trifluoromethanesulfonate (4.2 g, 44.50% yield) as a brown liquid. LC-MS (ES*): m z 404.11 [M+Na]+.

Step-5: To a stirred solution of 3-(3-methoxyphenyl)-1H-pyrazole (0.5 g, 2.87 mmol) in THF (10 mL) was added potassium tert-butoxide (644.16 mg, 5.74 mmol) at 0° C. and the mixture was stirred for 30 min before [1-[(4-methoxyphenyl)methyl]-2,6-dioxo-3-piperidyl]trifluoromethanesulfonate (2.19 g, 5.74 mmol) was added. The mixture was stirred at the same temperature for 2 h. After completion of the reaction, the reaction mixture was diluted with water (30 mL) and extracted with ethyl acetate (50 mL×2). The combined organic layer was dried over sodium sulfate and concentrated under reduced pressure to give the crude product, which was purified by flash column chromatography (Davisil silica) using a gradient of 5% methanol in dichloromethane to afford 1-[(4-methoxyphenyl)methyl]-3-[3-(3-methoxyphenyl)pyrazol-1-yl]piperidine-2,6-dione (0.37 g, 29% yield) as a pale yellow viscous liquid. LC-MS (ES*): m z 406.74 [M+H]+Step-6: To a stirred solution of 1-[(4-methoxyphenyl)methyl]-3-[3-(3-methoxyphenyl)pyrazol-1-yl]piperidine-2,6-dione (0.3 g, 0.739 mmol) in toluene (6 mL) was added trifluoromethanesulfonic acid (0.65 mL, 7.40 mmol) at 0° C. and stirred at 120° C. for 2 hr. After completion of the reaction, the reaction mixture was concentrated under reduced pressure to afford 3-[3-(3-hydroxyphenyl)pyrazol-1-yl]piperidine-2,6-dione (0.12 g, 14% yield) as brown viscous liquid. Without further purification used to next step. LC-MS (ES+): 15 m/z 272.17 [M+H]+.

Step-7: To a stirred solution of 3-[3-(3-hydroxyphenyl)pyrazol-1-yl]piperidine-2,6-dione (0.1 g, 0.368 mmol) in THF (2 mL) were added triethylamine (0.154 mL, 1.11 mmol), 1-(fluorosulfonyl)-2,3-dimethyl-1H-imidazol-3-ium trifluoromethanesulfonate (182.07 mg, 20 0.552 mmol) at room temperature and stirred for 1 h. After completion of the reaction, the reaction mixture was concentrated under reduced pressure to yield a crude residue, which was purified by prep-HPLC to afford 3-[3-(3-fluorosulfonyloxyphenyl)pyrazol-1-yl]-2,6-dioxo-piperidine (11.1 mg, 8.31% yield) as an off-white solid. LC-MS (ES*): m z 354.25 [M+H]+.

¹H-NMR (400 MHz, DMSO-d6): δ 11.11 (s, 1H), 8.0-7.90 (m, 3H), 7.63 (t, J=8.0 Hz, 1H), 7.52 (d, J=9.6 Hz, 1H ), 6.92 (d, J=2.4 Hz, 1H), 5.49-5.45 (m, 1H), 2.88-2.79 (m, 1H), 2.67-2.60 (m, 2H), 2.40-2.30 (m, 1H) ppm.¹⁹F NMR (376 MHz, DMSO-d6): δ 38.78 ppm.

Prep-HPLC Method: Column/dimensions: X-BRIDGE C18 (19*250*5 um); Mobile phase A: 0.1% TFA IN WATER(AQ); Mobile phase B: 100% Acetonitrile; Gradient (Time/% B): 0/20,2/20,13/75; Flow rate: 18 mL/min; Solubility: Acetonitrile+WATER+THF Synthesis A69: Synthesis of 2-amino-5-(1-(2,6-dioxopiperidin-3-yl)-1H-pyrazol-3-yl)phenyl sulfurofluoridate (A-72):

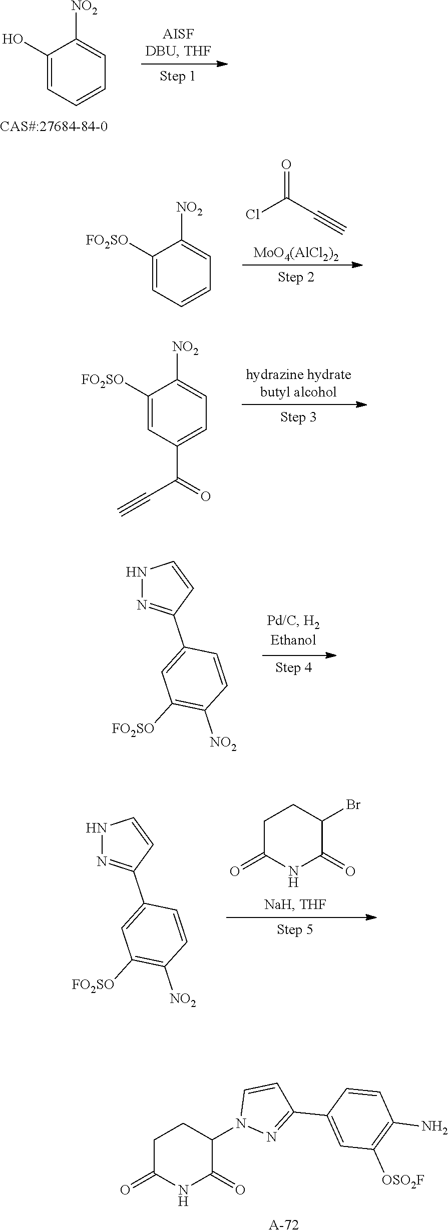

Step 1: 2-nitrophenyl sulfurofluoridate To a solution of 2-nitrophenol (CAS #:27684-84-0) in THF at room temperature is added DBU (2 eq) followed by AISF (1.5 eq) and the reaction stirred until judged complete.

After standard workup and purification, the intermediate would be concentrated under reduced pressure and purified under standard conditions to provide 2-nitrophenyl sulfurofluoridate. 10 Step 2: 2-nitro-5-propioloylphenyl sulfurofluoridate To a flask of 2-nitrophenyl sulfurofluoridate at room temperature is added propioloyl chloride (5 eq) followed by MoO4(AlCl2)2 (0.05 eq) and the reaction mixture is stirred at room temperature until judged complete. The reaction mixture would be filtered, and a standard work up performed to afford the crude product. The material would be purified in standard conditions to provide 2-nitro-5-propioloylphenyl sulfurofluoridate.

Step 3: 2-nitro-5-(1H-pyrazol-3-yl)phenyl sulfurofluoridate To a solution of 2-nitro-5-propioloylphenyl sulfurofluoridate in butyl alcohol at room temperature is added hydrazine hydrate (1 eq) and the reaction mixture is refluxed until judged complete. The reaction mixture would be filtered. The material would be purified in standard conditions to provide 2-nitro-5-(1H-pyrazol-3-yl)phenyl sulfurofluoridate.

Step 4: 2-amino-5-(1H-pyrazol-3-yl)phenyl sulfurofluoridate To a flask is added 2-nitro-5-(1H-pyrazol-3-yl)phenyl sulfurofluoridate (1 eq), in ethanol at room temperature is added 10% palladium on carbon (3 eq), and the reaction mixture is stirred at room temperature under H2 until completion of the reaction. It would be filtered through diatomaceous earth, concentrated under reduced pressure and subjected to standard purification to provide 2-amino-5-(1H-pyrazol-3-yl)phenyl sulfurofluoridate.

Step 5: 2-amino-5-(1-(2,6-dioxopiperidin-3-yl)-1H-pyrazol-3-yl)phenyl sulfurofluoridate (A-72) To a solution of sodium hydride (1.1 eq) in THF at 0° C. is added 2-amino-5-(1H-pyrazol-3-yl)phenyl sulfurofluoridate and the reaction mixture is stirred at 60° C. until judged complete. After, the reaction mixture would be cooled to room temperature and 3-bromopiperidine-2,6-dione (1.2 eq) would be added and the reaction mixture heated to 60° C. until judged complete. The reaction would be quenched with 20% IPA-DCM solution, washed 10 with water and brine. The organic layer would be dried with standard conditions and concentrated under reduced pressure to afford the crude material. Standard purification would provide 2-amino-5-(1-(2,6-dioxopiperidin-3-yl)-1H-pyrazol-3-yl)phenyl sulfurofluoridate (A-72).

Synthesis A70: Synthesis of 2-bromo-5-(1-(2,6-dioxopiperidin-3-yl)-1H-pyrazol-3-yl)phenyl sulfurofluoridate (A-73):

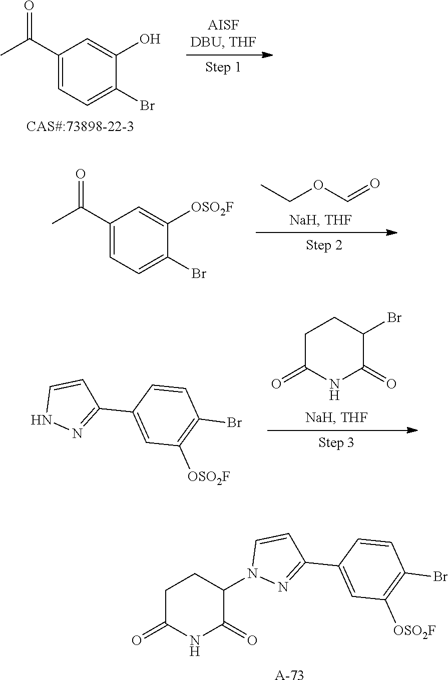

Step 1: 5-acetyl-2-bromophenyl sulfurofluoridate To a solution of 1-(4-bromo-3-hydroxyphenyl)ethan-1-one (CAS #:73898-22-3) in THF at room temperature is added DBU (2 eq) followed by AISF (1.5 eq) and the reaction is stirred until judged complete. After standard workup and purification, the intermediate is concentrated under reduced pressure and purified under standard conditions to provide 5-acetyl-2-bromophenyl sulfurofluoridate.

Step 2: 2-bromo-5-(1H-pyrazol-3-yl)phenyl sulfurofluoridate To a solution of sodium hydride (1.1 eq) in THF at 0° C. is added 5-acetyl-2-bromophenyl sulfurofluoridate and the reaction mixture is stirred until judged complete. After, the reaction mixture is cooled to room temperature and ethyl formate (1.2 eq) is added and 5 stirred until judged complete. The reaction is quenched with water and the aqueous layer is acidified with HCl and subjected to a standard workup. The crude material is dissolved in toluene and hydrazine hydrate (1.2 eq) is added and reaction is stirred at 95° C. until judged complete. Standard work-up and standard purification conditions would provide 2-bromo-5-(1H-pyrazol-3-yl)phenylsulfurofluoridate. 10 Step 3: 2-bromo-5-(1-(2,6-dioxopiperidin-3-yl)-1H-pyrazol-3-yl)phenyl sulfurofluoridate (A-73) To a solution of sodium hydride (1.1 eq) in THF at 0° C. is added 2-bromo-5-(1H-pyrazol-3-yl)phenyl sulfurofluoridate and the reaction mixture is stirred at 60° C. until judged complete. The reaction mixture is cooled to room temperature and 3-bromopiperidine-2,6-dione (1.2 eq) is added and the reaction mixture heated to 60° C. until judged complete. The reaction is quenched with 20% IPA-DCM solution and washed with water and brine. The organic layer is dried with standard conditions and concentrated under reduced pressure to afford the crude material. Standard purification provides 2-bromo-5-(1-(2,6-dioxopiperidin-3-yl)-1H-pyrazol-3-yl)phenylsulfurofluoridate (A-73).

Synthesis A71: Synthesis of 3-(1-(2,6-dioxopiperidin-3-yl)-1H-pyrazol-3-yl)benzenesulfonyl fluoride (A-74):

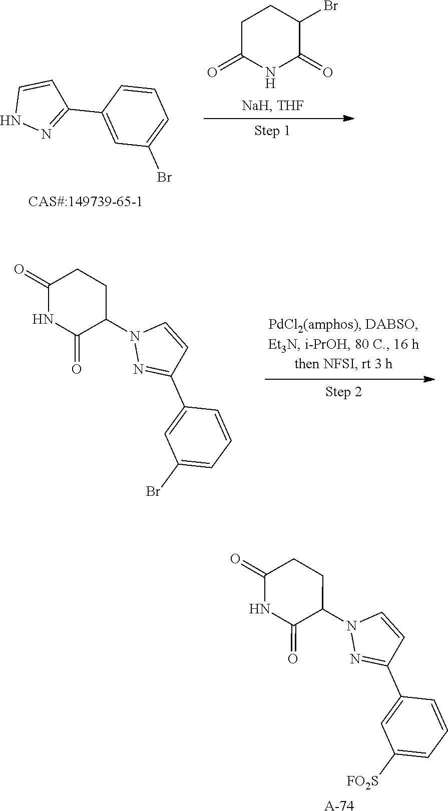

Step 1: 3-(3-(3-bromophenyl)-1H-pyrazol-1-yl)piperidine-2,6-dione To a solution of sodium hydride (1.1 eq) in THF at 0° C. is added 2-bromo-5-(1H-pyrazol-3-yl)phenyl sulfurofluoridate and the reaction mixture is stirred at 60° C. until judged complete. After, the reaction mixture would be cooled to room temperature and 3-bromopiperidine-2,6-dione (1.2 eq) would be added and the reaction mixture heated to 60° C. until judged complete. The reaction would be quenched with 20% IPA-DCM solution, washed with water and brine. The organic layer would be dried with standard conditions and concentrated under reduced pressure to afford the crude material. Standard purification would provide 2-bromo-5-(1-(2,6-dioxopiperidin-3-yl)-1H-pyrazol-3-yl)phenylsulfurofluoridate.

Step 2: 3-(1-(2,6-dioxopiperidin-3-yl)-1H-pyrazol-3-yl)benzenesulfonyl fluoride (A-74) To a solution of 2-bromo-5-(1-(2,6-dioxopiperidin-3-yl)-1H-pyrazol-3-yl)phenylsulfurofluoridate (1 eq) and DABSO (0.6 eq) in i-PrOH at room temperature is added triethylamine (3 eq) followed by purging with N2 for 5 minutes. Pd(amphos)C12 (0.05 eq) is 10 added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with sat. aq. sodium thiosulfate and a standard workup is performed, the intermediate is concentrated under reduced pressure and purified under standard conditions to provide 3-(1-(2,6-dioxopiperidin-3-yl)-1H-pyrazol-3-yl)benzenesulfonyl fluoride (A-74).

Synthesis A72: Synthesis of 3-(1-(2,6-dioxopiperidin-3-yl)-1H-imidazol-4-yl)phenyl sulfurofluoridate (A-75):

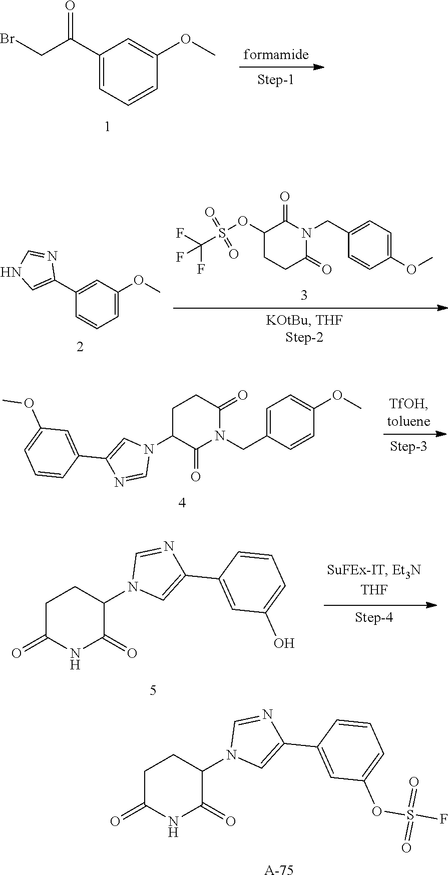

A solution of 2-bromo-1-(3-methoxyphenyl)ethanone 1 (3 g, 13.10 mmol) in formamide, 99% (6.24 mL, 157.16 mmol) was stirred for 2.5 h at 165° C. After completion of the reaction, the reaction mixture was diluted with water (30 mL) and extracted with ethyl acetate (20 mL×2). The aqueous layer was basified with 3 M NaOH solution (3 mL) and extracted with ethyl acetate (50 mL×2). The combined organic layer was dried over anhydrous sodium sulfate and concentrated under reduced pressure to afford 4-(3-methoxyphenyl)-1H-imidazole 2 (1.75 g, 74.82% yield) as a viscous liquid. LC-MS (ES*): m z 175.35 [M+H]+.

Step-2: To a stirred solution of 4-(3-methoxyphenyl)-1H-imidazole 2 (0.5 g, 2.87 mmol) in THF (5 mL) was added potassium tert-butoxide (644.16 mg, 5.74 mmol) at 0° C. and 10 the mixture was stirred for 1 h at room temperature. Then a solution of [1-[(4-methoxyphenyl)methyl]-2,6-dioxo-3-piperidyl]trifluoromethanesulfonate 3 (1.31 g, 3.44 mmol) in THF (5 mL) was added dropwise at 0° C. and stirred for 16 h at room temperature.

After completion of the reaction, the reaction mixture was quenched with sat. ammonium chloride solution (20 mL) and extracted with ethyl acetate (20 ml×2). The combined organic 15 layer was dried over anhydrous sodium sulfate and concentrated under reduced pressure to afford 3-[4-(3-methoxyphenyl)imidazol-1-yl]-1-[(4-methoxyphenyl)methyl]piperidine-2,6-dione 4 (0.6 g, 29.78% yield) as a blue viscous liquid. LC-MS (ES+): m z 406.09 [M+H]+.

Step-3: To a stirred solution of 3-[4-(3-methoxyphenyl)imidazol-1-yl]-1-[(4-methoxyphenyl)methyl]piperidine-2,6-dione 4 (0.35 g, 0.86 mmol) in toluene (5 mL) was added trifluoromethanesulfonic acid (0.75 mL, 8.63 mmol) at 0° C. and stirred for 2 h at 120° C. After completion of the reaction, the reaction mixture was diluted with water (20 mL) and extracted with ethyl acetate (5 mL×2). The separated aqueous layer was basified with sat. sodium bicarbonate (10 mL) solution and extracted with ethyl acetate (20 mL×2). The combined organic layer was dried over anhydrous sodium sulfate and concentrated under reduced pressure to afford 3-[4-(3-hydroxyphenyl) imidazol-1-yl]piperidine-2, 6-dione 5 (0.18 g, 20% yield) as a pale brown viscous liquid. LC-MS (ES+): m z 272.22 [M+H]+.

Step-4: To a stirred solution of 3-[4-(3-hydroxyphenyl)imidazol-1-yl]piperidine-2,6-dione 5 (0.17 g, 0.62 mmol) in acetonitrile (3 mL) was added triethylamine (0.18 mL, 1.25 mmol) at room temperature and stirred for 5 min. Then 1-(fluorosulfonyl)-2,3-dimethyl-1H-imidazol-3-ium trifluoromethanesulfonate (247.62 mg, 0.75 mmol) was added into the reaction mixture at 0° C. and stirred at room temperature for 1 h. After completion of the reaction, the reaction mixture was concentrated under reduced pressure to give the crude product, which was purified by achiral SFC purification to afford 3-[4-(3-fluorosulfonyloxyphenyl)imidazol-1-yl]-2,6-dioxo-piperidine A-75 (11 mg, 4.90% yield) as an off white solid. LC-MS (ES*): m z 354.12 [M+H]+. ¹H-NMR (400 MHz, DMSO-d6): δ 11.18 (brs, 1H), 7.91-7.87 (m, 3H), 7. 81 (d, J=1.20 Hz, 1H), 7. 60 (d, J=8.0 Hz, 1H), 7.42-7.39 (m, 1H), 5.38-5.32 (m, 1H), 2.88-2.79 (m, 1H), 2.71-2.66 (m, 1H), 2.64-2.57 (m, 1H), 2.29-2.24 (m, 1H) ppm. ¹⁹F NMR (376 MHz, DMSO-d6): δ 38.65 ppm. Preparative SFC Method: Column/dimensions: Ethyl Pyridine (30×250) mm,5; % C02: 70%; % Co solvent: 30% (Acetonitrile: Isopropyl alcohol) (1:1); Flow: 100 mL/min; Back, Pressure: 100 bar; Temperature: 30° C.; UV: 254 nm Synthesis A73: Synthesis of 2-bromo-5-(1-(2,6-dioxopiperidin-3-yl)-1H-imidazol-4-yl)phenyl sulfurofluoridate (A-76):

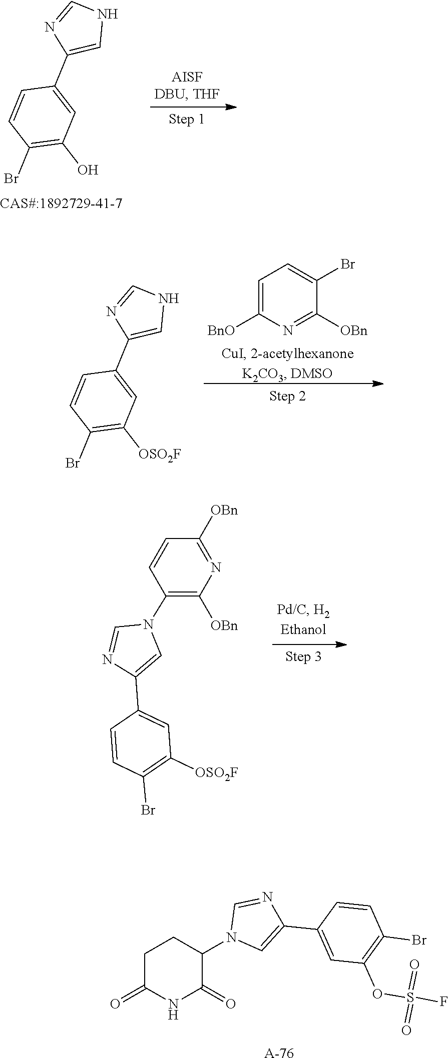

Step 1: To a solution of 2-Bromo-5-(1H-imidazol-5-yl)phenol (CAS #: 1892729-41-7) in THF at room temperature is added DBU (2 eq) followed by AISF (1.5 eq) and the reaction is stirred until judged complete. Standard workup and purification would provide 2-bromo-5-(1H-imidazol-4-yl)phenyl sulfurofluoridate.

Step 2: To a solution of 2-bromo-5-(1H-imidazol-4-yl)phenyl sulfurofluoridate (1.0 eq), 2,6-bis(benzyloxy)-3-bromopyridine (CAS #:16727-47-2, 1.0 eq) and K2CO3 (2.0 eq) in DMSO at room temperature is added CuI (0.15 eq) and 2-acetylhexanone (0.5 equiv) and the reaction mixture is stirred at 140° C. until judged complete. Standard workup and standard purification conditions would provide 5-(1-(2,6-bis(benzyloxy)pyridin-3-yl)-1H-imidazol-4-yl)-2-bromophenyl sulfurofluoridate.

Step 3: To a flask is added 5-(1-(2,6-bis(benzyloxy)pyridin-3-yl)-1H-imidazol-4-yl)-2-bromophenyl sulfurofluoridate(1 eq), in ethanol at room temperature is added 10% palladium on carbon (3 eq), and the reaction mixture is stirred at room temperature under H2 atmosphere until the reaction is judged complete. The reaction mixture is filtered through diatomaceous earth, concentrated under reduced pressure and subjected to standard purification to provide 2-bromo-5-(1-(2,6-dioxopiperidin-3-yl)-1H-imidazol-4-yl)phenyl sulfurofluoridate (A-76).

Synthesis A74: Synthesis of 3-(1-(2,6-dioxopiperidin-3-yl)-1H-imidazol-4-yl)benzenesulfonylfluoride (A-77):

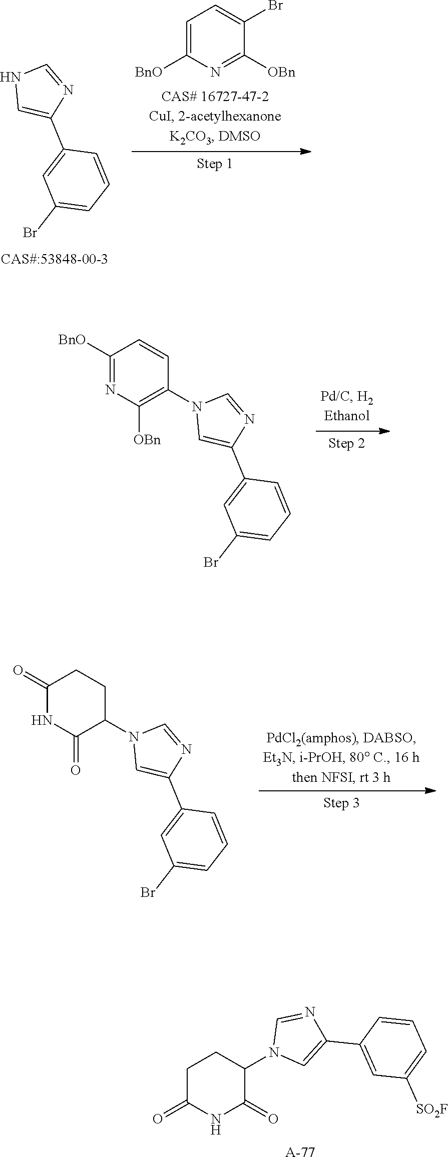

15 Step 1: 2,6-bis(benzyloxy)-3-(4-(3-bromophenyl)-1H-imidazol-1-yl)pyridine To a solution of 4-(3-bromophenyl)-1H-imidazole sulfurofluoridate (1.0 eq), 2,6-bis(benzyloxy)-3-bromopyridine (CAS #:16727-47-2, 1.0 eq) and K2CO3 (2.0 eq) in DMSO at room temperature is added CuI (0.15 eq) and 2-acetylhexanone (0.5 eq) and the reaction mixture is stirred at 140° C. until judged complete. Standard workup and standard purification conditions provide 2,6-bis(benzyloxy)-3-(4-(3-bromophenyl)-1H-imidazol-1-yl)pyridine.

Step 2: 3-(4-(3-bromophenyl)-1H-imidazol-1-yl)piperidine-2,6-dione To a flask is added 3-(1-(2,6-bis(benzyloxy)pyridin-3-yl)-1H-imidazol-4-yl)phenyl sulfurofluoridate (1 eq), in ethanol at room temperature is added 10% palladium on carbon (3 eq), and the reaction mixture is stirred at room temperature under H2 atmosphere until the reaction is judged complete. The reaction mixture is filtered through diatomaceous earth, concentrated under reduced pressure and subjected to standard purification to provide 3-(4-(3-bromophenyl)-1H-imidazol-1-yl)piperidine-2,6-dione.

Step 3: 3-(1-(2,6-dioxopiperidin-3-yl)-1H-imidazol-4-yl)benzenesulfonylfluoride (A-77) To a solution of 3-(4-(3-bromophenyl)-1H-imidazol-1-yl)piperidine-2,6-dione (1 eq) and DABSO (0.6 eq) in i-PrOH at room temperature is added triethylamine (3 eq) followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room 10 temperature until judged complete. The reaction is quenched with sat. aq. sodium thiosulfate and a standard workup is performed, the intermediate is concentrated under reduced pressure and purified under standard conditions to provide 3-(1-(2,6-dioxopiperidin-3-yl)-1H-imidazol-4-yl)benzenesulfonylfluoride (A-77). 15 Synthesis A75: Synthesis of 1-(2,6-dioxopiperidin-3-yl)indolin-4-yl sulfurofluoridate (A-78):

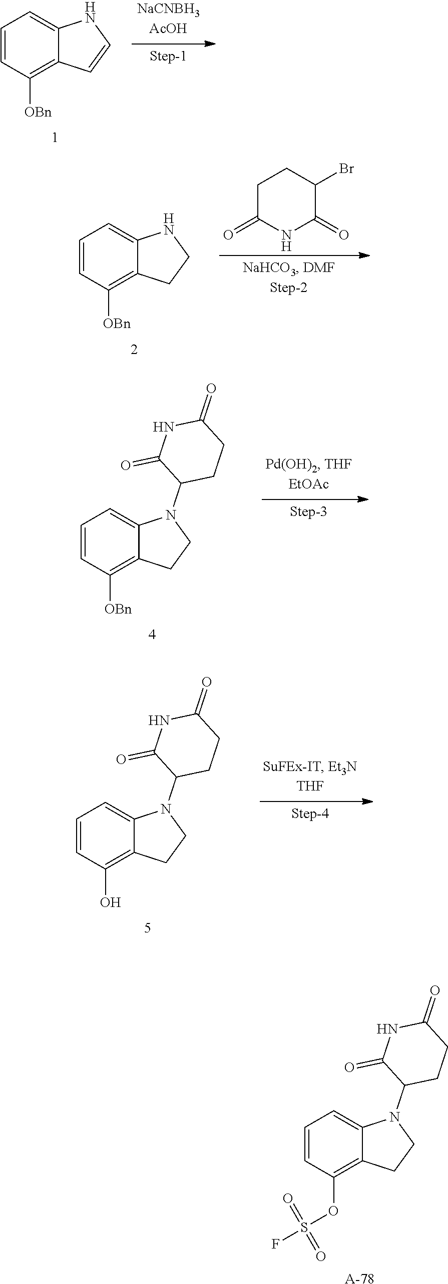

Step-1: To a stirred solution of 4-benzyloxy-1H-indole 1 (3 g, 13.44 mmol) in acetic acid (30 mL) was added sodium cyanoborohydride (1.69 g, 26.87 mmol) at 0° C. and stirred for 5 h at room temperature. After completion of reaction, the reaction mixture was concentrated under reduced pressure and the obtained residue was basified with sat. sodium hydroxide solution, diluted with water (40 mL) and extracted with ethyl acetate (50 mL×3). The combined organic layer was dried over anhydrous sodium sulfate and concentrated under reduced pressure to afford 4-benzyloxyindoline 2 (2.2 g, 45.07% yield) as a pale-yellow gummy. LC-MS (ES*): m z 226.09 [M+H]+.

Step-2: To a stirred solution of 4-benzyloxyindoline 2 (2 g, 8.88 mmol) in DMF (20 mL) was added NaHCO₃ (2.98 g, 35.51 mmol) and stirred for 15 min before 3-bromopiperidine-2,6-dione 3 (3.41 g, 17.76 mmol) was added and the reaction mixture was stirred at 90° C. for 16 h. After completion of the reaction, the reaction mixture was diluted with ice cold water (50 mL) and the obtained precipitate was filtered and dried under vacuum to afford 3-(4-benzyloxyindolin-1-yl)piperidine-2,6-dione 4 (2.4 g, 68.03% yield) as a pale green solid. LCMS (ES*): m z 337.1 [M+H]+.

Step-3: A stirred solution of 3-(4-benzyloxyindolin-1-yl)piperidine-2,6-dione 4 (1.0 g, 2.97 mmol) in THF (10 mL) and ethyl acetate (10 mL) was degassed with nitrogen gas for 5 min, then Palladium hydroxide (417.48 mg, 2.97 mmol) was added into the reaction mixture and stirred under hydrogen atmosphere (60 psi) at room temperature for 16 h. After completion of reaction, the reaction mixture was filtered through a pad of celite, which was subsequently washed with THF (20 mL). The filtrate was concentrated under reduced pressure to afford 3-(4-hydroxyindolin-1-yl)piperidine-2,6-dione 5 (0.6 g, 58.32% yield) as a pale green gummy. LCMS (ES*): m z 247.23 [M+H]+.

Step-4: To a stirred solution of 3-(4-hydroxyindolin-1-yl)piperidine-2,6-dione 5 (0.1 g, 0.406 mmol) in acetonitrile (3 mL) were added triethylamine (169 pL, 1.22 mmol) and 1-(fluorosulfonyl)-2,3-dimethyl-1H-imidazol-3-ium trifluoromethanesulfonate (267 mg, 0.812 mmol) at room temperature and stirred for 1 h. After completion of the reaction, the solvent was removed under reduced pressure and the obtained crude residue was purified by Prep-HPLC to afford 1-(2,6-dioxo-3-piperidyl)-4-fluorosulfonyloxy-indoline A-78 (15.6 mg, 12% yield) as an off-white solid. LCMS (ES*): m z 329.12 [M+H]+. ¹H NMR (400 MHz, DMSO-d6): 6 10.87 (s, 1H), 7.14 (t, J=8.2 Hz, 1H), 6.70-6.55 (m, 2H), 4.75-4.71 (m, 1H), 3.65-3.50 (m, 1H), 3.45-3.40 (m, 1H), 3.20-2.95 (m, 2H), 2.85-2.70 (m, 1H), 2.65-2.55 (m, 1H), 2.30-2.15 (m, 1H), 2.0-1.90 (m, 1H) ppm. Prep-HPLC Method: Column/dimensions: X-SELECT C18 (19*150*5 um); Mobile phase A: 0.1% Formic acid in water; Mobile phase B: 100% Acetonitrile; Gradient (Time/% B): 0/20, 2/25, 15/75; Flow rate: 18 mL/min; Solubility: Acetonitrile+Water+THF.

Synthesis A76: Synthesis of 6-bromo-1-(2,6-dioxopiperidin-3-yl)indolin-4-yl sulfurofluoridate (A-79)

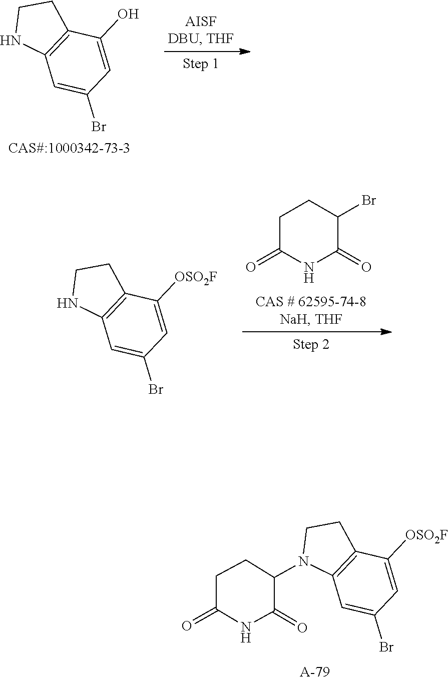

5 Step 1: To a solution of 6-bromoindolin-4-ol (CAS #: 1000342-73-3, 1 eq) in THF at room temperature is added DBU (2 eq) followed by AISF (1.5 eq) and the reaction is stirred until judged complete. Standard workup and purification provide 6-bromoindolin-4-yl sulfurofluoridate. 10 Step 2: To a solution of sodium hydride (1.1 eq) in THF at 0° C. is added 6-bromoindolin-4-yl sulfurofluoridate and the reaction mixture is stirred at 60° C. until judged complete. The reaction mixture is cooled to room temperature and 3-bromopiperidine-2,6-dione (CAS #62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material. Standard purification provides 6-bromo-1-(2,6-dioxopiperidin-3-yl)indolin-4-yl sulfurofluoridate (A-79).

Synthesis A77: Synthesis of 1-(2,6-dioxopiperidin-3-yl)indoline-4-sulfonyl fluoride (A-80):

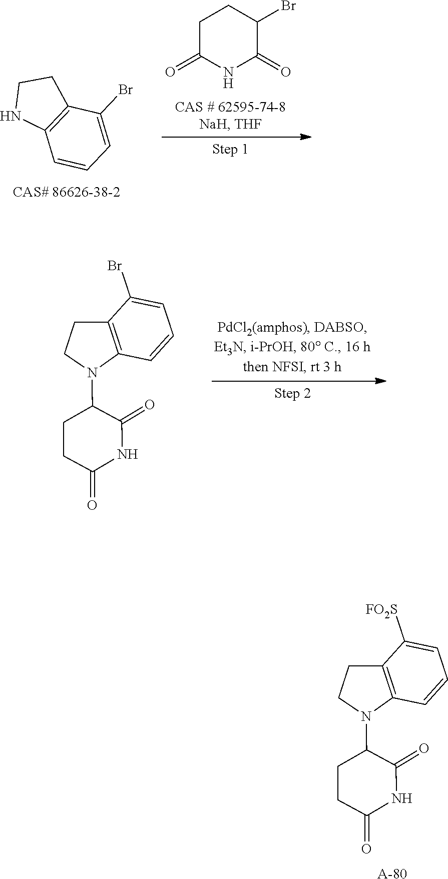

Step 1: 3-(4-bromoindolin-1-yl)piperidine-2,6-dione To a solution of sodium hydride (1.1 eq) in THF at 0° C. is added 4-bromoindoline (CAS #86626-38-2, 1 eq) and the reaction mixture is stirred at 60° C. until judged complete.

The reaction mixture is cooled to room temperature and 3-bromopiperidine-2,6-dione (CAS #5 62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material. Standard purification provides 3-(4-bromoindolin-1-yl)piperidine-2,6-dione.

Step 2: 1-(2,6-dioxopiperidin-3-yl)indoline-4-sulfonyl fluoride (A-80) To a solution of 3-(4-bromoindolin-1-yl)piperidine-2,6-dione (1 eq) and DABSO (0.6 eq) in i-PrOH at room temperature is added triethylamine (3 eq) followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room 15 temperature until judged complete. The reaction is quenched with sat. aq. sodium thiosulfate and a standard workup is performed, the intermediate is concentrated under reduced pressure and purified under standard conditions to provide 1-(2,6-dioxopiperidin-3-yl)indoline-4-sulfonyl fluoride (A-80).

Synthesis A78: Synthesis of 1-(2,6-dioxopiperidin-3-yl)-2-methyl-1H-indol-5-yl sulfurofluoridate (A-81):

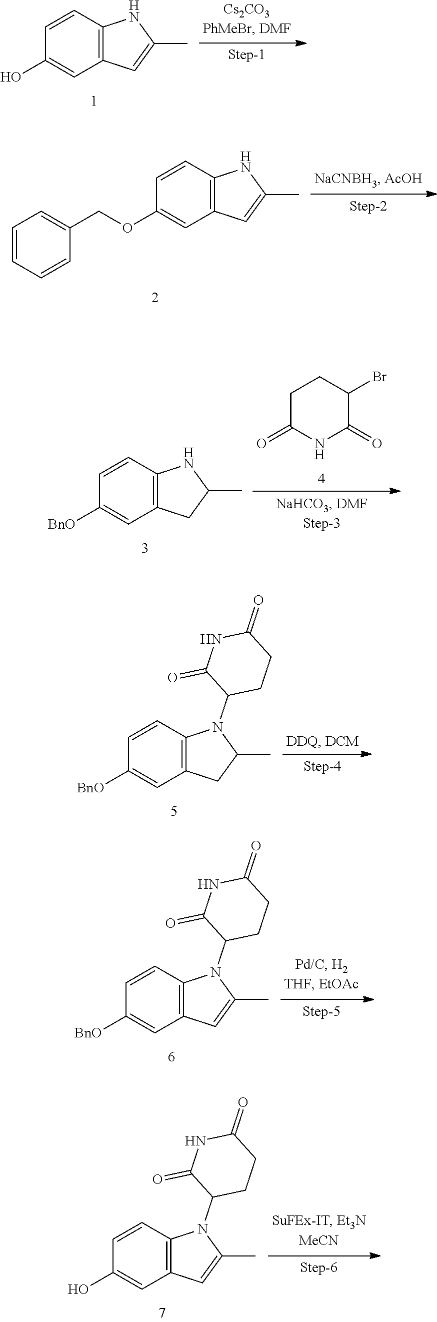

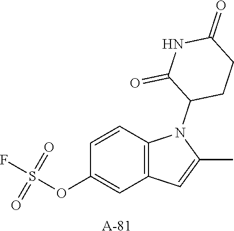

Step-1:

To a stirred solution of 2-methyl-1H-indol-5-ol 1 (3 g, 20.38 mmol) in DMF (30 mL) was added cesium carbonate (13.28 g, 40.76 mmol) at room temperature and stirred for 5 5 min. Then, bromomethylbenzene (2.42 mL, 20.38 mmol) was added at 0° C. and the reaction mixture was stirred at room temperature for 16 h. After completion of the reaction, the reaction mixture was diluted with ice cold water (50 mL) and extracted with ethyl acetate (20 mL×3). The combined organic layer was dried over anhydrous sodium sulfate and concentrated under reduced pressure to give the crude product, which was purified by column chromatography (Davisil silica) using 10% ethyl acetate in pet ether as an eluent to afford 5-benzyloxy-2-methyl-1H-indole 2 (4 g, 72.72% yield) as pale yellow solid. LC-MS (ES*): m z 238.06 [M+H]+.

Step-2: To a stirred solution of 5-benzyloxy-2-methyl-1H-indole 2 (2 g, 8.43 mmol) in acetic 15 acid (20 mL) was added sodium cyanoborohydride (1.06 g, 16.86 mmol) at 0° C. and stirred for 16 h at room temperature. After completion of the reaction, the reaction mixture was concentrated under reduced pressure and the obtained residue was basified with sat. sodium hydroxide solution, diluted with water (40 mL) and extracted with ethyl acetate (20 mL×3). The combined organic layer was dried over anhydrous sodium 20 sulfate and concentrated under reduced pressure to afford 5-benzyloxy-2-methyl-indoline 3 (2 g, 77.67% yield) as a pale-yellow gummy. LC-MS (ES+): m z 240.00 [M+H]+.

Step-3: To a stirred solution of 5-benzyloxy-2-methyl-indoline 3 (2 g, 8.36 mmol) in DMF (15.01 mL) was added sodium bicarbonate (2.11 g, 25.07 mmol) and stirred for 5 min. Then 3-bromopiperidine-2,6-dione 4 (3.21 g, 16.71 mmol) was added and the reaction mixture was stirred at 90° C. for 16 h. After completion of the reaction, ice cold water (20 mL) was added into the reaction mixture, and the resulting precipitate was filtered and dried under vacuum to afford 3-(5-benzyloxy-2-methyl-indolin-1-yl)piperidine-2,6-dione 5 (1.7 g, 26.66% yield) as a blue solid. LC-MS (ES+): m z 351.31 [M+H]+.

Step-4: To a stirred solution of 3-(5-benzyloxy-2-methyl-indolin-1-yl)piperidine-2,6-dione 5 (0.5 g, 1.43 mmol) in DCM (10 mL) was added DDQ (323 mg, 1.43 mmol) at 0° C. and stirred for 1 h at the same temperature. After completion of the reaction, the reaction mixture was 5 diluted with sat. sodium bicarbonate solution (30 mL) and extracted with DCM (15 mL×3).

The combined organic layer was dried over anhydrous sodium sulfate and concentrated under reduced pressure to afford 3-(5-benzyloxy-2-methyl-indol-1-yl)piperidine-2,6-dione 6 (0.35 g, 62% yield) as a red solid. LC-MS (ES*): m z 349.10 [M+H]+.

Step-5: A stirred solution of 3-(5-benzyloxy-2-methyl-indol-1-yl)piperidine-2,6-dione 6 (0.35 g, 1.00 mmol) in THF (3 mL) and ethyl acetate (3 mL) was degassed with nitrogen gas for 5 min. Then palladium on carbon (320.73 mg, 3.01 mmol) was added into the reaction mixture and stirred under hydrogen atmosphere (balloon pressure) at room temperature for 16 h. After completion of reaction, the reaction mixture was filtered through a pad of celite, which was subsequently washed with THF (20 mL). The filtrate was concentrated under reduced pressure to afford 3-(5-hydroxy-2-methyl-indol-1-yl)piperidine-2,6-dione 7 (0.25 g, 56.30% yield) as red brown gummy. LC-MS (ES*): m z 259.20 [M+H]+.

Step-6: To a stirred solution of 3-(5-hydroxy-2-methyl-indol-1-yl)piperidine-2,6-dione 7 (0.15 20 g, 580.78 mol) in acetonitrile (3 mL) was added triethylamine (0.16 mL, 1.16 mmol) at room temperature and stirred for 5 min before 1-(fluorosulfonyl)-2,3-dimethyl-1H-imidazol-3-ium trifluoromethanesulfonate (286.85 mg, 871.17 mol) was added at 0° C. The reaction mixture was stirred at room temperature for 1 h. After completion of the reaction, the reaction mixture was concentrated under reduced pressure to give the crude product, which was purified by Prep HPLC to afford 1-(2,6-dioxo-3-piperidyl)-5-fluorosulfonyloxy-2-methyl-indole A-81 (24.6 mg, 12% yield) as an off white solid. LC-MS (ES*): m z 341.13 [M+H]+. ¹H-NMR (400 MHz, DMSO-d6): δ 11.18 (s, 1H), 7. 65 (d, J=2.0 Hz, 1H), 7.29 (brs, 1H), 7.15 (brs, 1H), 6.40 (s, 1H), 5.05 (brs, 1H), 2.94-2.90 (m, 1H), 2.75-2.66 (m, 2H), 2.41 (brs, 3H), 2.10-2.07 (m, 1H) ppm. ¹⁹F NMR (376 MHz, DMSO-d6): δ 36.90 ppm. Prep-HPLC Method: Column/dimensions: X-BRIDGE C18 (19×250 x Sum); Mobile phase A: 0.1% Formic acid in water (Aq) Mobile phase B: 100% Acetonitrile; Gradient (Time/% B): 0/20, 2/25, 14/80; Flow rate: 18 mL/min; Solubility: Acetonitrile+Water+Tetrahydrofuran Synthesis A79: Synthesis of 6-bromo-1-(2,6-dioxopiperidin-3-yl)-2-methyl-1H-indol-5-yl sulfurofluoridate (A-82):

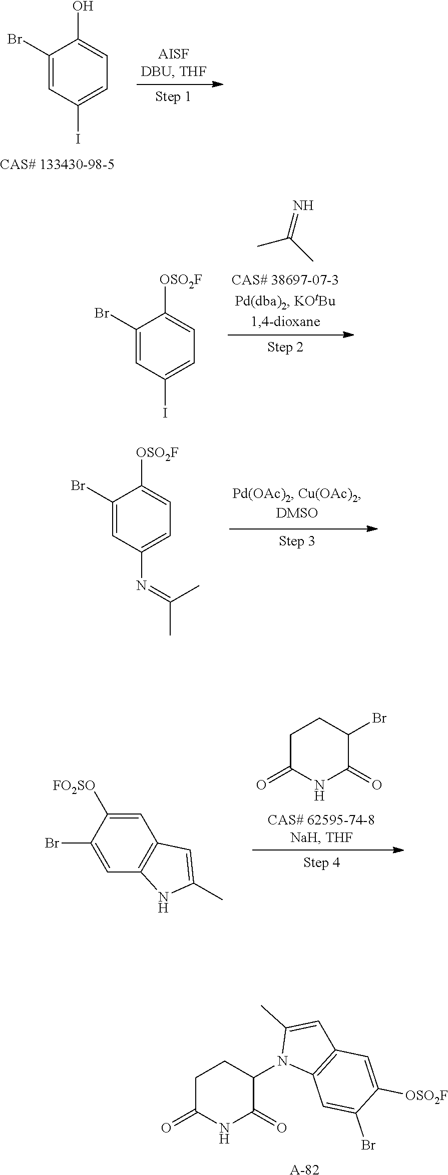

Step 1: 2-bromo-4-iodophenyl sulfurofluoridate To a solution of 2-bromo-4-iodophenol (CAS #133430-98-5, 1 eq) in THF at room temperature is added DBU (2 eq) followed by AISF (1.5 eq) and the reaction is stirred until judged complete. Standard workup and purification would provide 2-bromo-4-iodophenyl sulfurofluoridate. 10 Step 2: 2-bromo-4-(propan-2-ylideneamino)phenyl sulfurofluoridate To a solution of 2-bromo-4-iodophenyl sulfurofluoridate (1 eq), t-BuOK (1 eq) and propan-2-imine (CAS #38697-07-3, 1.05 eq) in 1,4-dioxane at room temperature is Pd(dba)2 (0.10 eq) is added, and the reaction mixture is heated to reflux until judged complete. The reaction is cooled to room temperature and a standard purification is directly performed to provide 2-bromo-4-(propan-2-ylideneamino)phenyl sulfurofluoridate.

Step 3: 6-bromo-2-methyl-1H-indol-5-yl sulfurofluoridate A solution of 2-bromo-4-(propan-2-ylideneamino)phenyl sulfurofluoridate (1 eq), Pd(OAc)2 (0.10 eq) and Cu(OAc)2 (3.0 eq) in DMSO at room temperature and the reaction mixture is heated to 40° C. until judged complete. The reaction is cooled and a standard work-up and purification is performed to provide 6-bromo-2-methyl-1H-indol-5-yl sulfurofluoridate. Step 4: 6-bromo-1-(2,6-dioxopiperidin-3-yl)-2-methyl-1H-indol-5-yl sulfurofluoridate (A-82) To a solution of sodium hydride (1.1 eq) in THF at 0° C. is added 6-bromo-2-methyl-1H-indol-5-yl sulfurofluoridate (1 eq) and the reaction mixture is stirred at 60° C. until judged 5 complete. The reaction mixture is cooled to room temperature and 3-bromopiperidine-2,6-dione (CAS #62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material.

Standard purification provides 6-bromo-1-(2,6-dioxopiperidin-3-yl)-2-methyl-1H-indol-5-yl sulfurofluoridate (A-82).

Synthesis A80: Synthesis of 1-(2,6-dioxopiperidin-3-yl)-2-methyl-1H-indole-5-sulfonyl fluoride (A-83):

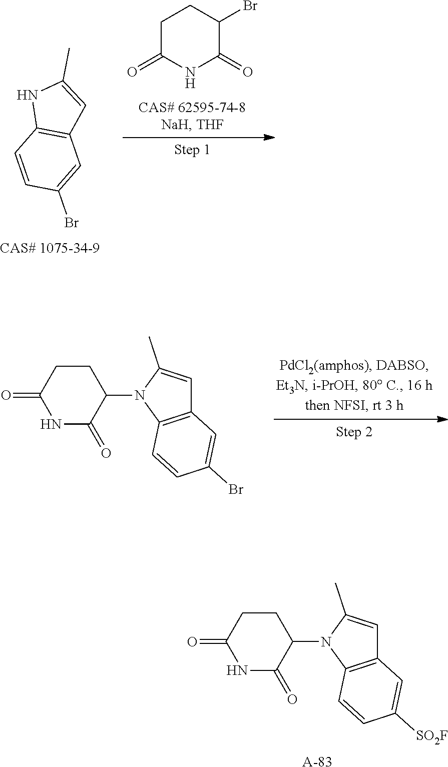 [SMILES: Cc1cc2cc(Br)ccc2[nH]1 ; O=C1CCC(Br)C(=O)N1 ; Cc1cc2cc(Br)ccc2n1C1CCC(=O)NC1=O ; Cc1cc2cc(S(=O)(=O)F)ccc2n1C1CCC(=O)NC1=O ; "A-83"]

1-(2,6-dioxopiperidin-3-yl)-2-methyl-1H-indole-5-sulfonyl fluoride (A-83) can be prepared in a similar manner to A-80, except replacing 4-bromoindoline with 5-bromo-2-methyl-1H-indole (CAS #1075-34-9).

Synthesis A81: Synthesis of 1-(2,6-dioxopiperidin-3-yl)-3-methyl-1H-indazol-6-yl 20 sulfurofluoridate (A-84):

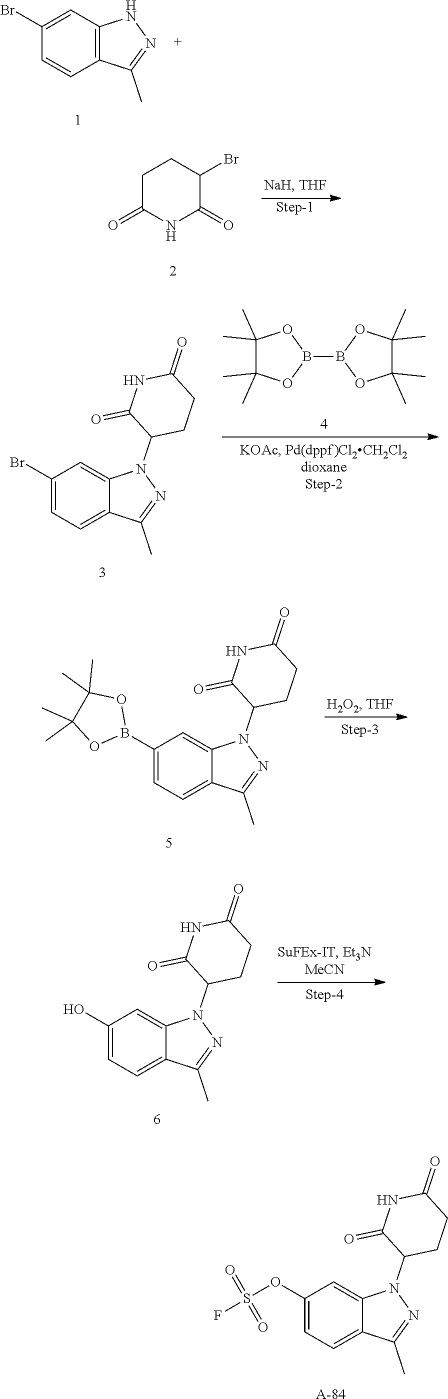

Step-1:

To a stirred solution of 6-bromo-3-methyl-1H-indazole 1 (0.2 g, 0.947 mmol) in THF (10 mL) was added sodium hydride (60% dispersion in oil) (379.01 mg, 9.48 mmol) at 0° C. and the reaction mixture was stirred at 70° C. for 1 h. Then 3-bromopiperidine-2,6-dione 2 (909.75 mg, 4.74 mmol) in THF (5 mL) was added dropwise at 0° C. The reaction mixture was stirred at 70° C. under nitrogen atmosphere for 16 h. After completion of the reaction, the reaction mixture was quenched with saturated ammonium chloride solution (10 mL), diluted with water (20 mL) and extracted ethyl acetate(20 mL×2). The combined organic layer was 10 dried over sodium sulfate and concentrated under reduced pressure to give the crude product, which was purified by flash column chromatography (using Davisil silica and 30% ethyl acetate in pet ether as eluent) to afford 3-(6-bromo-3-methyl-indazol-1-yl)piperidine-2,6-dione 3 (0.12 g, 39.21% yield) as a brown liquid. LC-MS (ES*): m z 322.17 [M+H]+Step-2: To a stirred solution of 3-(6-bromo-3-methyl-indazol-1-yl)piperidine-2,6-dione 3 (0.1 g, 0.310 mmol) and 4,4,5,5-tetramethyl-2-(4,4,5,5-tetramethyl-1,3,2-dioxaborolan-2-yl)-1,3,2-dioxaborolane 4 (118.24 mg, 0.465 mmol) in dioxane (5 mL) was added potassium acetate (91.39 mg, 0.931 mmol) at room temperature. The reaction was degassed with nitrogen gas for 10 minutes before addition of Pd(dppf)Cl12CH2Cl2 (25.35 mg, 0.031 20 mmol) and stirred at 100° C. for 16 h. After completion of the reaction, the reaction mixture was diluted with ice cold water (10 mL) and extracted with ethyl acetate (20 mL×2). The combined organic layer was dried over anhydrous sodium sulfate and concentrated under reduced pressure to afford 3-[3-methyl-6-(4,4,5,5-tetramethyl-1,3,2-dioxaborolan-2-yl)indazol-1-yl]piperidine-2,6-dione 5 (0.15 g, 91.54% yield) as a brown solid. LCMS (ES+): 25 m z 370.39 [M+H]+Step-3: To a stirred solution of 3-[3-methyl-6-(4,4,5,5-tetramethyl-1,3,2-dioxaborolan-2-yl)indazol-1-yl]piperidine-2,6-dione 5 (0.15 g, 0.406 mmol) in THF (4.49 mL) was added hydrogen peroxide 35% (0.375 mL, 12.24 mmol) at 0° C. and stirred at room temperature for 3 h. After completion, the reaction mixture was diluted with water (10 mL) and extracted with ethyl acetate (20 mL×2). The combined organic layer was washed with aqueous NaHSO4 (20 mL) solution, and concentrated under reduced pressure. The crude product was triturated with diethyl ether (5 mL×2), dried under reduced pressure to afford 3-(6-hydroxy-3-methyl-indazol-1-yl)piperidine-2,6-dione 6 (0.06 g, 41.65% yield) as an off-brown solid. LCMS (ES*): m z 260.0 [M+H]+Step-4: To a stirred solution of 3-(6-hydroxy-3-methyl-indazol-1-yl)piperidine-2,6-dione 6 (60 mg, 0.231 mmol) in acetonitrile (3 mL) were added Et3N (96.77 L, 0.694 mmol) and 1-(fluorosulfonyl)-2,3-dimethyl-1H-imidazol-3-ium trifluoromethanesulfonate (152.40 mg, 0.462 mmol) at 0° C. and stirred for 1 h at room temperature. After completion of reaction, the reaction mixture was diluted with water (20 mL) and extracted with ethyl acetate (20 mL×2). The combined organic layer was dried over anhydrous sodium sulfate and concentrated under reduced pressure to give the crude product, which was purified by Prep HPLC to afford 1-(2,6-dioxo-3-piperidyl)-6-fluorosulfonyloxy-3-methyl-indazole A-84 (13.7 mg, 17.06% yield) as an off-white solid. LC-MS (ES+): m/z 342.12 [M+H]+. ¹H-NMR (400 MHz, DMSO-d6): δ 11.11 (s, 1H), 7.97-7.94 (m, 2H), 7.33 (dd, J=10.40, Hz, 1H), 5.85-5.81 (m, 1H), 2.90-2.70 (m, 3H), 2.52 (s, 3H), 2.31-2.26 (m, 1H) ppm. ¹⁹F NMR (376 MHz, DMSO-d6): δ 38.661 ppm. Prep HPLC Method: Column/dimensions: X-Bridge C18 (19*250*5 p); Mobile phase A: 0.1% formic acid in water; Mobile phase B: 100% Acetonitrile; Gradient (Time/% B):0/15,2/20,12.5/75; Flow rate; 18 ml/min; Solubility Acetonitrile+THF+H20.

Synthesis A82: Synthesis of 5-bromo-1-(2,6-dioxopiperidin-3-yl)-3-methyl-1H-indazol-6-yl sulfurofluoridate (A-85):

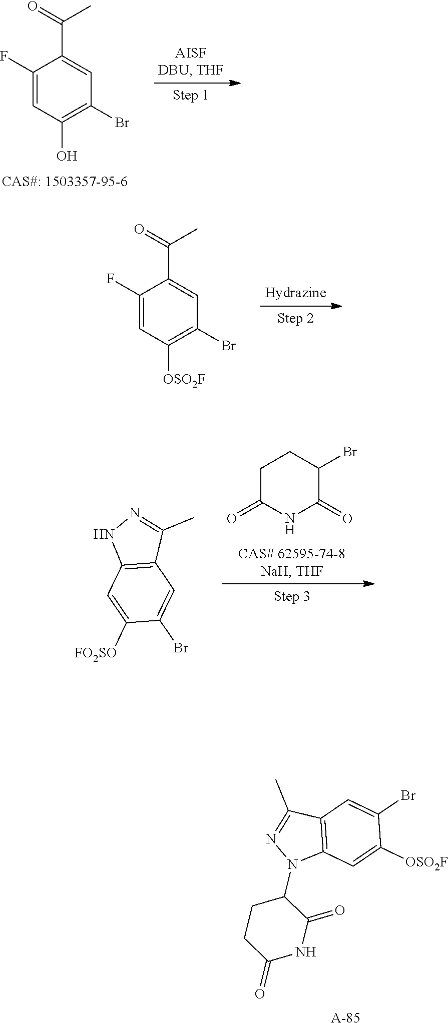

Step 1: 4-acetyl-2-bromo-5-fluorophenyl sulfurofluoridate To a solution of 1-(5-bromo-2-fluoro-4-hydroxyphenyl)ethan-1-one (CAS #1503357-95-6, 1 eq) in THF at room temperature is added DBU (2 eq) followed by AISF (1.5 eq) and the reaction is stirred until judged complete. Standard workup and purification would provide 4-acetyl-2-bromo-5-fluorophenyl sulfurofluoridate.

Step 2: 5-bromo-3-methyl-1H-indazol-6-yl sulfurofluoridate A solution of 4-acetyl-2-bromo-5-fluorophenyl sulfurofluoridate (1 eq) in solvent quantities of anhydrous hydrazine is heated to 120° C. until judged complete. The reaction mixture is concentrated under reduced pressure and a standard workup is performed. A standard purification provides 5-bromo-3-methyl-iH-indazol-6-yl sulfurofluoridate.

Step 3: 5-bromo-1-(2,6-dioxopiperidin-3-yl)-3-methyl-1H-indazol-6-yl sulfurofluoridate (A-85) To a solution of sodium hydride (1.1 eq) in THF at 0° C. is added provide 5-bromo-3-methyl-1H-indazol-6-yl sulfurofluoridate (1 eq) and the reaction mixture is stirred at 60° C. 15 until judged complete. The reaction mixture is cooled to room temperature and 3-bromopiperidine-2,6-dione (CAS #62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material. Standard purification provides 5-bromo-1-(2,6-dioxopiperidin-3-yl)-3-methyl-iH-indazol-6-yl sulfurofluoridate (A-85).

Synthesis A83: Synthesis of 1-(2,6-dioxopiperidin-3-yl)-3-methyl-1H-indazole-6-sulfonyl fluoride (A-86): Br 0 N 0 N H PdCl₂(amphos), DABSO, HN CAS #62595-74-8 N- Et₃N, i-PrOH, 80° C., 16 h N-NaH, THF N/then NFSI, rt, 3 h, Step 1 Step 2 0 N O 0 N O Br H Br H SO₂F CAS #7746-27-2 A-86

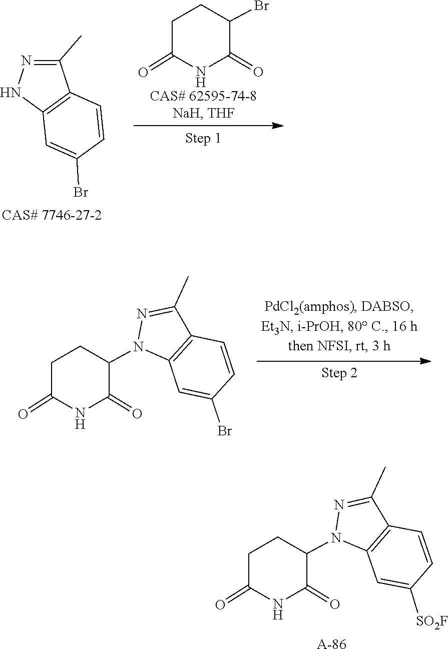

1-(2,6-dioxopiperidin-3-yl)-3-methyl-1H-indazole-6-sulfonylfluoride (A-86) can be^{P}GP-⁷¹³,C³ prepared in a similar manner to A-80, except replacing 4-bromoindoline with 6-bromo-3-methyl-1H-indazole (CAS #7746-27-2).

Synthesis A84: Synthesis of 1-(2,6-dioxopiperidin-3-yl)-1H-benzo[d]imidazol-5-yl sulfurofluoridate (A-87):

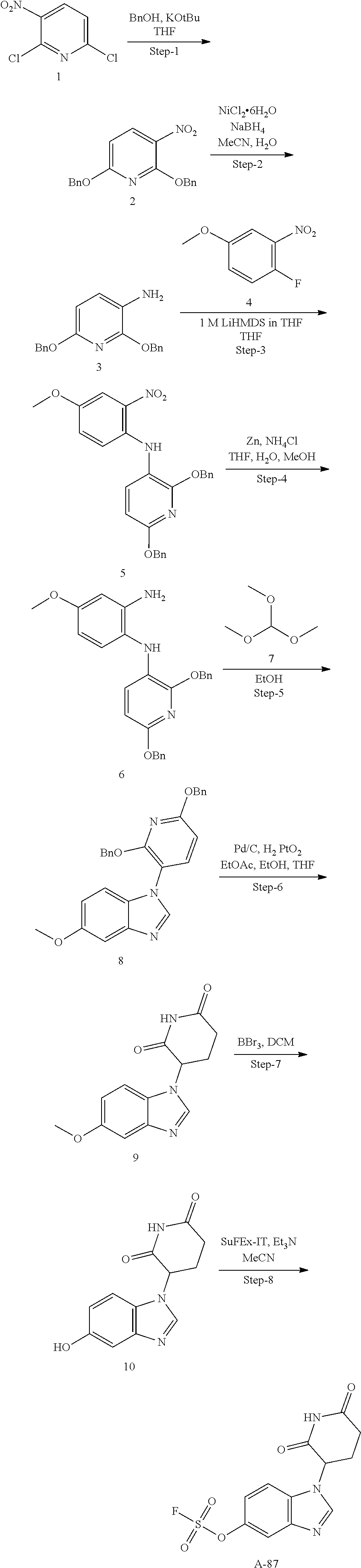

Step-1: To a stirred solution of benzyl alcohol (193.03 mL, 1.87 mol) in THF (500 mL) added potassium tert-butoxide (235.49 g, 2.10 mol) at 0° C. and stirred for 1 h. Then a solution of 2,6-dichloro-3-nitro-pyridine 1 (150 g, 777.25 mmol) in THF (1000 mL) was added 10 at −20° C. and stirred for 1 h at the same temperature. After completion of the reaction, the reaction mixture was poured into ice-cold water (1 L), and the obtained precipitate was filtered and dried under reduced pressure to afford 2,6-dibenzyloxy-3-nitro-pyridine 2 (140 g, 53.55% yield) as a yellow solid. LCMS (ES*): m z 337.0 [M+H]+Step-2: To a stirred solution of 2,6-dibenzyloxy-3-nitro-pyridine 2 (140 g, 416.24 mmol) in acetonitrile (1200 mL) and water (200 mL) was added Nickel (II) chloride hexahydrate (19.79 g, 83.25 mmol) before sodium borohydride (31.50 g, 832.49 mmol) was added portion wise at 0° C. and stirred for 4 h at room temperature. After completion of the reaction, the reaction mixture was concentrated under reduced pressure. The residue was 20 diluted with water (1000 mL) and extracted with ethyl acetate (1000 mL×3). The combined organic layer was dried over anhydrous sodium sulfate, concentrated under reduced pressure to give the crude product, which was purified by flash column chromatography (Davisil silica and 40% ethyl acetate in pet ether as eluent) to afford 2,6-dibenzyloxypyridin-3-amine 3 (90 g, 67.05% yield) as a brown liquid. LCMS (ES*): m z 307.20 [M+H]+Step-3: To a stirred solution of 1-fluoro-4-methoxy-2-nitrobenzene 4 (3 g, 17.53 mmol) in THF (50 mL) was added 1 M LiHMDS in THF (70.12 mL, 35.06 mmol) at −78° C. The reaction mixture was stirred at −78° C. for 1 h before 2,6-bis(benzyloxy)pyridin-3-amine 3 (5.91 g, 19.28 mmol) was added at −78° C. and stirred at room temperature for 15 h. The reaction mixture was quenched with ammonium chloride solution in water (100 mL) and extracted with ethyl acetate (250 mL×2). The combined organic layer was washed 10 with brine solution (50 mL), dried over anhydrous sodium sulfate, and concentrated under reduced pressure to afford 2,6-bis(benzyloxy)—N-(4-methoxy-2-nitrophenyl)pyridin-3-amine 5 (6 g, 60.10% yield) as a brown semi-solid. LC-MS (ES+): m z 458.13 [M+H]+Step-4: To a stirred solution of 2,6-dibenzyloxy-N-(4-methoxy-2-nitro-phenyl)pyridin-3-amine 5 (5 g, 10.93 mmol) in THF (50 mL) were added Zinc (7.15 g, 109.30 mmol) and NH4C1 (5.85 g, 109.30 mmol) in water (50 mL) dropwise at 0° C. The reaction mixture was stirred at 50° C. for 16 h. After completion of the reaction, the reaction mixture was filtered through a pad of celite, which was subsequently washed with ethyl acetate (20 mL). The filtrate was diluted with water (50 mL) and extracted with ethyl acetate (50 mL×2). The combined organic layer was dried over sodium sulfate and concentrated under reduced pressure to afford N1-(2,6-dibenzyloxy-3-pyridyl)-4-methoxy-benzene-1,2-diamine 6 (5 g, 54.01% yield) as a brown liquid. LC-MS (ES+): m z 428.46 [M+H]+Step-5: To a stirred solution of N1-(2,6-dibenzyloxy-3-pyridyl)-4-methoxy-benzene-1,2-diamine 6 (5 g, 11.70 mmol) in ethanol (5 mL) was added trimethoxymethane 7 (6.41 mL, 58.48 mmol) and stirred at 100° C. for 16 h. After completion of the reaction, the reaction mixture was concentrated under reduced pressure to give the crude product, which was purified by column chromatography (using 100-200 silica, ~60% ethyl acetate in pet ether as an eluent) to afford 1-(2,6-dibenzyloxy-3-pyridyl)-5-methoxy-benzimidazole 8 (2 g, 35.00% yield) as a brown semi solid. LC-MS (ES+): m z 438.97 [M+H]+.

Step-6: To a stirred solution of 1-(2,6-dibenzyloxy-3-pyridyl)-5-methoxy-benzimidazole 8 (2 g, 4.57 mmol) in ethyl acetate (50 mL), THF (50 mL), ethanol (50 mL) were added 10% Palladium on carbon, wet (4.00 g, 37.59 mmol) and Platinum dioxide (400.00 mg, 1.76 mmol). The reaction mixture was stirred under hydrogen atmosphere in a Parr shaker reactor (80 psi) at room temperature for 16 h. After completion of the reaction, the reaction mixture was filtered through a pad of celite, which was subsequently washed with ethyl acetate (50 mL). The filtrate was concentrated under reduced pressure to give 3-(5-methoxybenzimidazol-1-yl)piperidine-2,6-dione 9 (800 mg, 64.01% yield). LC-MS (ES*): m z 260.22 [M+H]+Step-7: To a stirred solution of 3-(5-methoxybenzimidazol-1-yl)piperidine-2,6-dione 9 (0.55 g, 2.12 mmol) in DCM (20 mL) added boron tribromide (1.5 ml, 14.85 mmol) at 0° C. and stirred at room temperature for 2 hr. After completion of reaction, the reaction mixture was diluted 10 with ice cold water (20 mL) and washed with DCM (20 mL×2). The product was highly polar, and the aqueous layer was concentrated under reduced pressure to afford 3-(5-hydroxybenzimidazol-1-yl)piperidine-2,6-dione 10 (0.5 g, 75.93% yield) as an off-white solid. LC-MS (ES+): m z 245.96 [M+H]+Step-8: To a stirred solution of 3-(5-hydroxybenzimidazol-1-yl)piperidine-2,6-dione 10 (500 mg, 2.04 mmol) in acetonitrile (4.34 mL) were added triethylamine (0.85 mL, 6.12 mmol) and 1-(fluorosulfonyl)-2,3-dimethyl-1H-imidazol-3-ium trifluoromethanesulfonate (1.34 g, 4.08 mmol) at 0° C. and stirred at room temperature for 1 h. After completion of reaction, the reaction mixture was concentrated under reduced pressure to give the crude product, which was purified by Prep HPLC to afford 1-(2,6-dioxo-3-piperidyl)-5-fluorosulfonyloxy-benzimidazole A-87 (40.52 mg, 6.02% yield) as an off-white solid. LC-MS (ES+): m/z 328.10 [M+H]+. ¹H NMR (400 MHz, DMSO-d6): δ 11.24 (s, 1H), 8.50 (s, 1H), 7.98 (d, J=2.00 Hz, 1H), 7.77 (d, J=8.80 Hz, 1H), 7.50 (dd, J=11.20, Hz, 1H), 5.80-5.76 (m, 1H), 2.90-2.60 (m, 3H), 2.31-2.30 (m, 1H) ppm. Prep HPLC method: Column/dimensions: X-SELECT C18 (10×250 x Sum) Mobile phase A:0.1% formic acid in water Mobile phase B: Acetonitrile Gradient (Time/% B):0/10, 2/10, 7/60, 9.7/60, 9.8/100 Flow rate: 8 ml/min.

Solubility: Water+Acetonitrile+THF.

Synthesis A85: Synthesis of 6-bromo-1-(2,6-dioxopiperidin-3-yl)-1H-benzo[d]imidazol-5-yl sulfurofluoridate (A-88):

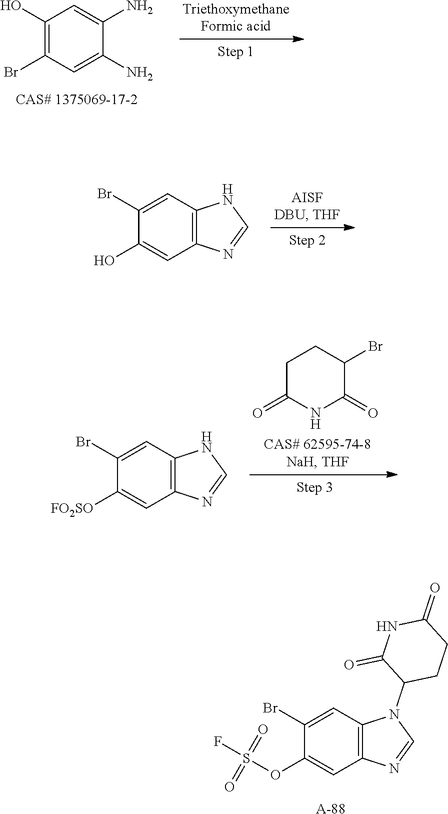

Step 1: 6-bromo-1H-benzo[d]imidazol-5-ol To a solution of 4,5-diamino-2-bromophenol (CAS #1375069-17-2, 1 eq) in formic acid is added triethoxymethane (20 eq) at room temperature and the reaction is stirred and 5 heated at 90° C. until judged complete. The reaction is concentrated under reduced pressure and purified with a stand purification to provide 6-bromo-1H-benzo[d]imidazol-5-ol.

Step 2: 6-bromo-1H-benzo[d]imidazol-5-yl sulfurofluoridate To a solution of 6-bromo-1H-benzo[d]imidazol-5-ol (1 eq) in THF at room temperature is added DBU (2 eq) followed by AISF (1.5 eq) and the reaction is stirred until judged 10 complete. Standard workup and purification would provide 6-bromo-1H-benzo[d]imidazol-5-yl sulfurofluoridate.

Step 3: 6-bromo-1-(2,6-dioxopiperidin-3-yl)-1H-benzo[d]imidazol-5-yl sulfurofluoridate (A-88) To a solution of sodium hydride (1.1 eq) in THF at 0° C. is added provide 6-bromo-1H-benzo[d]imidazol-5-yl sulfurofluoridate (1 eq) and the reaction mixture is stirred at 60° C. until judged complete. The reaction mixture is cooled to room temperature and 3-bromopiperidine-2,6-dione (CAS #62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material.

Standard purification provides 6-bromo-1-(2,6-dioxopiperidin-3-yl)-1H-benzo[d]imidazol-5-yl sulfurofluoridate (A-88).

Synthesis A86: Synthesis of 1-(2,6-dioxopiperidin-3-yl)-1H-benzo[d]imidazole-5-sulfonyl fluoride (A-89):

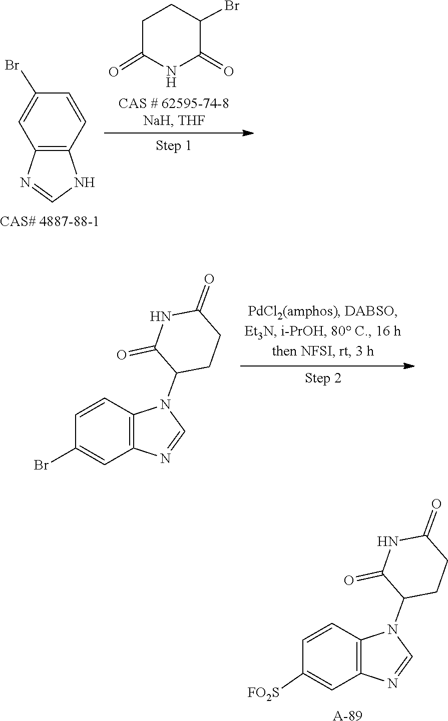

1-(2,6-dioxopiperidin-3-yl)-1H-benzo[d]imidazole-5-sulfonyl fluoride (A-89) can be 5 prepared in a similar manner to A-80, except replacing 4-bromoindoline with 5-bromo-1H-benzo[d]imidazole (CAS #4887-88-1).

Synthesis A87: Synthesis of 1-(2,6-dioxopiperidin-3-yl)-1H-pyrrolo[2,3-b]pyridin-5-yl sulfurofluoridate (A-90):

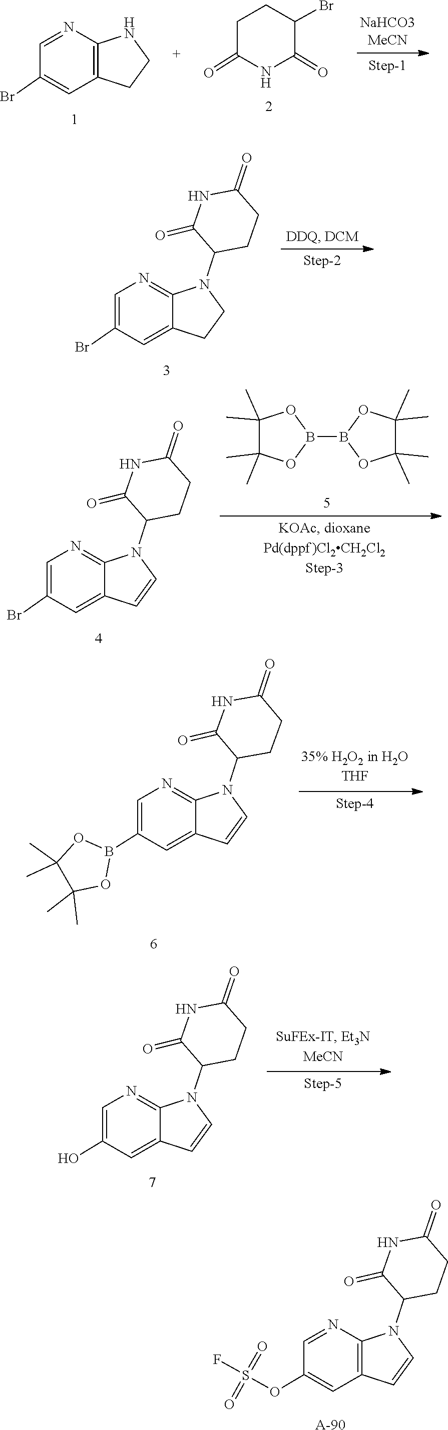

Step-1: To a stirred solution of 5-bromo-2,3-dihydro-1H-pyrrolo[2,3-b]pyridine 1 (1.7 g, 8.54 mmol) and 3-bromopiperidine-2,6-dione 2 (8.20 g, 42.70 mmol) in acetonitrile (20 mL) was added NaHCO₃ (7.17 g, 85.41 mmol) at room temperature. The reaction mixture was stirred at 100° C. for 48 h. After completion of the reaction, the reaction mixture was quenched with saturated ammonium chloride solution (30 mL), diluted with water (50 mL) and extracted with ethyl acetate (100 mL×2). The combined organic layer was dried over sodium sulfate and concentrated under reduced pressure to afford 3-(5-bromo-2,3-dihydropyrrolo[2,3-b]pyridin-1-yl)piperidine-2,6-dione 3 (760 mg, 18.25% yield) as a brown liquid. LC-MS (ES*): m z 312.05 [M+H]+Step-2: To a stirred solution of 3-(5-bromo-2,3-dihydropyrrolo[2,3-b]pyridin-1-yl)piperidine-2,6-dione 3 (0.76 g, 2.45 mmol) in DCM (10 mL) was added 4,5-dichloro-3,6-dioxo-cyclohexa-1,4-diene-1,2-dicarbonitrile (556.25 mg, 2.45 mmol) at 0° C. and stirred for 1 h at the same temperature. After completion of the reaction, the reaction mixture was diluted with sat. sodium bicarbonate solution (30 mL) and extracted with DCM (25 mL×3). The combined organic layer was dried over anhydrous sodium sulfate and concentrated under reduced pressure to give the crude product, which was triturated with diethyl ether (20 mL), dried under reduced pressure to afford 3-(5-bromopyrrolo[2,3-b]pyridin-1-yl)piperidine-2,6-dione 4 (0.23 g, 28.39% yield) as a red solid. LC-MS (ES+): m z 309.09 [M+H]+Step-3: To a stirred solution of 3-(5-bromopyrrolo[2,3-b]pyridin-1-yl)piperidine-2,6-dione 4 (0.23 g, 0.746 mmol) and 4,4,5,5-tetramethyl-2-(4,4,5,5-tetramethyl-1,3,2-dioxaborolan-2-yl)-1,3,2-dioxaborolane 5 (284.32 mg, 1.12 mmol) in dioxane (5 mL) was added potassium acetate (219.77 mg, 2.24 mmol) at room temperature and the mixture was degassed with nitrogen gas for 10 minutes. Then Pd(dppf)Cl2CH2Cl2 (60.96 mg, 0.074 mmol) was added and the reaction mixture was stirred at 90° C. for 16 h. After completion of the reaction, the reaction mixture was filtered through a pad of celite, which was subsequently washed with ethyl acetate (30 mL). The filtrate was concentrated under reduced pressure to afford 3-[5-(4,4,5,5-tetramethyl-1,3,2-dioxaborolan-2-yl)pyrrolo[2,3-b]pyridin-1-yl]piperidine-2,6-dione 6 (230 mg, 31.51% yield) as a brown gum. LC-MS (ES+): m z 356.52 [M+H]+Step-4: To a stirred solution of 3-[5-(4,4,5,5-tetramethyl-1,3,2-dioxaborolan-2-yl)pyrrolo[2,3-b]pyridin-1-yl]piperidine-2,6-dione 6 (0.23 g, 0.647 mmol) in tetrahydrofuran (5 mL) was added 35% hydrogen peroxide in water (1.15 mL, 37.53 mmol) at 0° C. and stirred at room temperature for 2 hr. After completion of the reaction, the reaction mixture was diluted with ethyl acetate (10 mL) and washed with NaHSO4 solution and concentrate under reduced pressure to get crude, which was washed with diethyl ether (10 mL) and dried under reduced pressure to afford 3-(5-hydroxypyrrolo[2,3-b]pyridin-1-yl)piperidine-2,6-dione 7 (0.27 g, 99.08% yield) as a brown solid. LC-MS (ES*): m z 246.11 [M+H]+Step-5: To a stirred solution of 3-(5-hydroxypyrrolo[2,3-b]pyridin-1-yl)piperidine-2,6-dione 7 (0.27 g, 1.10 mmol) in acetonitrile (3 mL) was added triethylamine (0.460 mL, 3.30 mmol) and 1-(fluorosulfonyl)-2,3-dimethyl-1H-imidazol-3-ium trifluoromethanesulfonate (725.05 mg, 2.20 mmol) at 0° C. and stirred the reaction mixture at room temperature for 1 h. After completion of the reaction, the reaction mixture was concentrated under reduced pressure to give the crude product, which was purified by SFC to afford 1-(2,6-dioxo-3-piperidyl)-5-fluorosulfonyloxy-pyrrolo[2,3-b]pyridine A-90 (23.4 mg, 6.35% yield) as a white 15 solid. LC-MS (ES+): m z 328.13 [M+H]+. ¹H-NMR (400 MHz, DMSO-d6): δ 11.12 (s, 1H), 8.50 (d, J=2.4 Hz, 1H), 8.37 (d, J=2.4 Hz, 1H), 7.83 (d, J=3.6 Hz, 1H), 6.69 (d, J=3.6 Hz, 1H), 5.85-5.81 (m, 1H), 3.03-2.94 (m, 1H), 2.88-2.81 (m, 1H), 2.78-2.65 (m, 1H), 2.20-2.18 (m, 1H) ppm. Preparative SFC Method: Column/dimensions: Ethyl Pyridine (30x250)mm,5[t, % C02: 85%, % Co solvent: 15% (ACN:IPA)(1:1), Flow: 100 ml/min, Back Pressure: 100 20 bar, Temperature: 30 0 C, UV: 220 nm, Solubility: acetonitrile, No. of injections: 12, Total purification time: 02:00 Hrs, Run time per injection: 06:00 Mins, Instrument details: Make/Model: ANL-BLR SFC-004.

Synthesis A88: Synthesis of 4-bromo-1-(2,6-dioxopiperidin-3-yl)-1H-pyrrolo[2,3-b]pyridin-5-yl sulfurofluoridate (A-91):

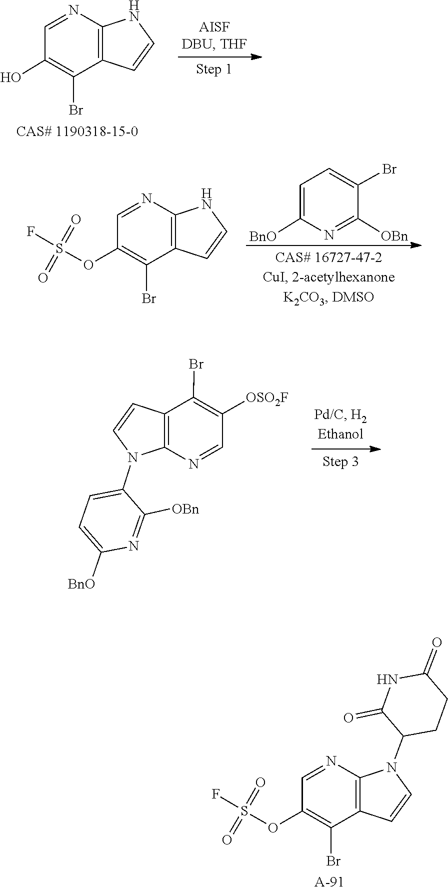

Step 1: 4-bromo-1H-pyrrolo[2,3-b]pyridin-5-yl sulfurofluoridate To a solution of 4-bromo-1H-pyrrolo[2,3-b]pyridin-5-ol (CAS #1190318-15-0, 1 eq) in THF at room temperature is added DBU (2 eq) followed by AISF (1.5 eq) and the reaction is 5 stirred until judged complete. Standard workup and purification would provide 4-bromo-1H-pyrrolo[2,3-b]pyridin-5-yl sulfurofluoridate.

Step 2: 1-(2,6-bis(benzyloxy)pyridin-3-yl)-4-bromo-1H-pyrrolo[2,3-b]pyridin-5-yl sulfurofluoridate To a solution of 4-bromo-1H-pyrrolo[2,3-b]pyridin-5-yl sulfurofluoridate (1.0 eq), 2,6-bis(benzyloxy)-3-bromopyridine (CAS #:16727-47-2, 1.0 eq) and K2CO3 (2.0 eq) in DMSO at room temperature is added CuI (0.15 eq) and 2-acetylhexanone (0.5 equiv) and the reaction mixture is stirred at 140° C. until judged complete. Standard workup and standard purification conditions provide 1-(2,6-bis(benzyloxy)pyridin-3-yl)-4-bromo-1H-pyrrolo[2,3-b]pyridin-5-yl sulfurofluoridate.

Step 3: 4-bromo-1-(2,6-dioxopiperidin-3-yl)-1H-pyrrolo[2,3-b]pyridin-5-yl sulfurofluoridate (A-91) To a flask is added 1-(2,6-bis(benzyloxy)pyridin-3-yl)-4-bromo-1H-pyrrolo[2,3-b]pyridin-5-yl sulfurofluoridate (1 eq), in ethanol at room temperature is added 10% palladium on carbon (3 eq), and the reaction mixture is stirred at room temperature under H2 atmosphere until the reaction is judged complete. The reaction mixture is filtered through diatomaceous earth, concentrated under reduced pressure and subjected to standard purification to provide 4-bromo-1-(2,6-dioxopiperidin-3-yl)-1H-pyrrolo[2,3-b]pyridin-5-yl sulfurofluoridate (A-91). Synthesis A89: Synthesis of 1-(2,6-dioxopiperidin-3-yl)-1H-pyrrolo[2,3-b]pyridine-5-sulfonyl fluoride (A-92):

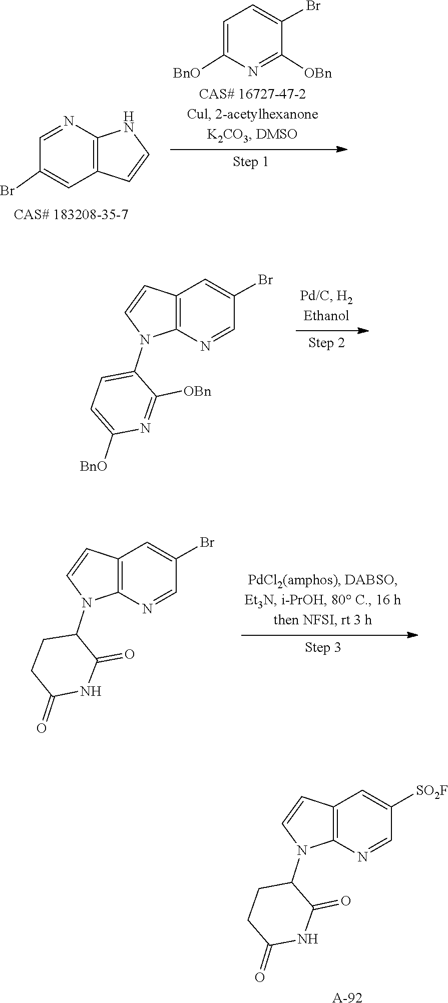 [SMILES: Brc1ccc(OCc2ccccc2)nc1OCc1ccccc1 ; Brc1cnc2[nH]ccc2c1 ; Brc1cnc2c(ccn2-c2ccc(OCc3ccccc3)nc2OCc2ccccc2)c1 ; O=C1CCC(n2ccc3cc(Br)cnc32)C(=O)N1 ; O=C1CCC(n2ccc3cc(S(=O)(=O)F)cnc32)C(=O)N1 ; "A-92"]

Step 1: 1-(2,6-bis(benzyloxy)pyridin-3-yl)-5-bromo-1H-pyrrolo[2,3-b]pyridine To a solution of 5-bromo-1H-pyrrolo[2,3-b]pyridine (CAS #183208-35-7, 1.0 eq), 2,6-bis(benzyloxy)-3-bromopyridine (CAS #16727-47-2, 1.0 eq) and K2CO3 (2.0 eq) in DMSO at room temperature is added CuI (0.15 eq) and 2-acetylhexanone (0.5 eq) and the reaction mixture is stirred at 140° C. until judged complete. Standard workup and standard purification conditions provide 1-(2,6-bis(benzyloxy)pyridin-3-yl)-5-bromo-1H-pyrrolo[2,3-b]pyridine.

Step 2: 3-(5-bromo-1H-pyrrolo[2,3-b]pyridin-1-yl)piperidine-2,6-dione To a flask is added 1-(2,6-bis(benzyloxy)pyridin-3-yl)-5-bromo-1H-pyrrolo[2,3-b]pyridine (1 eq), in ethanol at room temperature is added 10% palladium on carbon (3 eq), and the reaction mixture is stirred at room temperature under H2 atmosphere until the reaction is judged complete. The reaction mixture is filtered through diatonaceous earth, concentrated under reduced pressure and subjected to standard purification to provide 3-(5-bromo-1H-pyrrolo[2,3-b]pyridin-1-yl)piperidine-2,6-dione.

Step 3: 1-(2,6-dioxopiperidin-3-yl)-1H-pyrrolo[2,3-b]pyridine-5-sulfonyl fluoride (A-92) To a solution of 3-(5-bromo-1H-pyrrolo[2,3-b]pyridin-1-yl)piperidine-2,6-dione (1 eq) 20 and DABSO (0.6 eq) in i-PrOH at room temperature is added triethylamine (3 eq) followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with sat. aq. sodium thiosulfate and a standard workup is performed, the intermediate is concentrated under reduced pressure and purified under standard conditions to provide 1-(2,6-dioxopiperidin-3-yl)-1H-pyrrolo[2,3-b]pyridine-5-sulfonyl fluoride (A-92).

Synthesis A90: Synthesis of 2-(2,6-dioxopiperidin-3-yl)-1-oxo-1,2-dihydroisoquinolin-7-yl sulfurofluoridate (A-93):

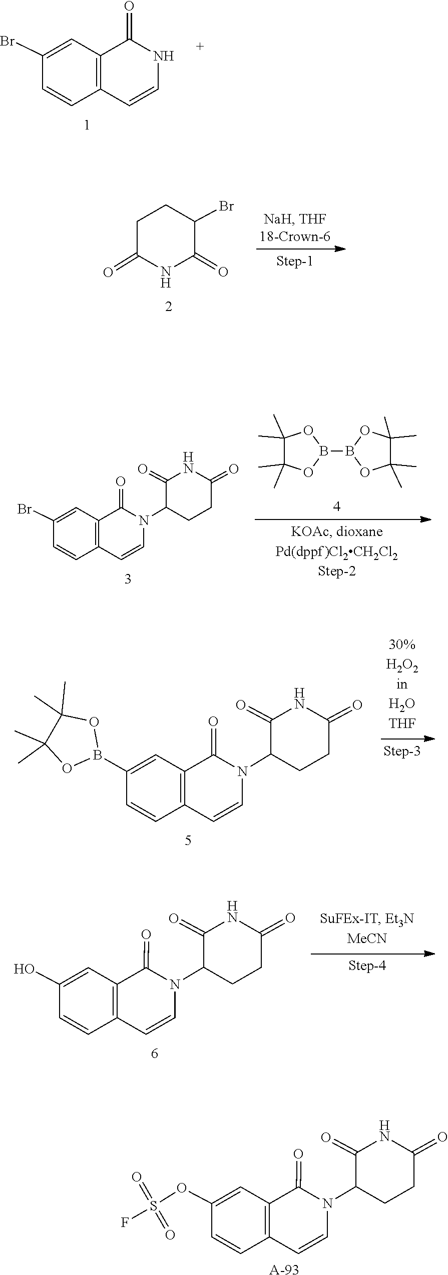

Step-1: To a stirred solution of 7-bromo-2H-isoquinolin-1-one 1 (1.0 g, 4.46 mmol) in THF (100 mL) were added sodium hydride (60% dispersion in mineral oil) (1.07 g, 44.63 mmol) and 18-Crown-6 (0.1 g, 0.378 mmol) at 0° C. The reaction mixture was stirred at 60° C. for 30 min before a solution of 3-bromopiperidine-2,6-dione 2 (4.28 g, 22.32 mmol) in THF (50 mL) was added slowly and the reaction mixture was stirred at 70° C. for 16 h. After completion of the reaction, the reaction mixture was concentrated under reduced pressure and the residue was diluted with ice cold water (50 mL) and extracted with ethyl acetate (100 mL×2). The combined organic layer was dried over anhydrous sodium sulfate and concentrated under 20 reduced pressure to give the crude product, which was purified by column chromatography (Davisil silica with ~63% of ethyl acetate in pet ether as an eluent) to afford 3-(7-bromo-1-oxo-2-isoquinolyl)piperidine-2,6-dione 3 (0.5 g, 25% yield) as off-white solid. LC-MS (ES*): m1z337.13 [M+H]+Step-2: To a stirred solution of 3-(7-bromo-1-oxo-2-isoquinolyl)piperidine-2,6-dione 3 (0.5 g, 1.49 mmol) in dioxane (20 mL) were added 4,4,5,5-tetramethyl-2-(4,4,5,5-tetramethyl-1,3,2-dioxaborolan-2-yl)-1,3,2-dioxaborolane 4 (757.69 mg, 2.98 mmol), potassium acetate (439 mg, 5 4.48 mmol) and the mixture was degassed with argon gas for 10 min. Then bis(1-diphenylphosphanylcyclopenta-2,4-dien-1-yl)iron;dichloromethane;dichloropalladium (1.22 g, 1.49 mmol) was added and the reaction mixture was stirred at 80° C. for 16 h. After completion of the reaction, the reaction mixture was filtered through a pad of celite, which was subsequently washed with ethyl acetate (100 mL). The filtrate was concentrated under reduced pressure to afford 3-[1-oxo-7-(4,4,5,5-tetramethyl-1,3,2-dioxaborolan-2-yl)-2-isoquinolyl]piperidine-2,6-dione 5 (0.3 g, 28% yield)as a reddish-brown liquid. LC-MS (ES*): m z 383.230 [M+H]+Step-3: To a stirred solution of 3-[1-oxo-7-(4,4,5,5-tetramethyl-1,3,2-dioxaborolan-2-yl)-2-isoquinolyl]piperidine-2,6-dione 5 (0.3 g, 0.784 mmol) in THF (10 mL) added 30% hydrogen peroxide solution in water (2 mL, 19.58 mmol) at 0° C. The reaction mixture was stirred at room temperature for 2 h. After completion of the reaction, the reaction mixture was quenched with sodium bisulfate solution (100 mL) and extracted with ethyl acetate (100 mL×3). The combined organic layer was dried over sodium sulfate and concentrated under reduced pressure to afford 3-(7-hydroxy-1-oxo-2-isoquinolyl)piperidine-2,6-dione 6 (0.150 g, 69% yield) as a yellow semi-solid. LCMS (ES-): m z 270.7[M - H]-Step-4: To a stirred solution of 3-(7-hydroxy-1-oxo-2-isoquinolyl)piperidine-2,6-dione 6 (0.150 g, 550.95 mol) in acetonitrile (5 mL) were added triethylamine (191 pL, 1.38 mmol) and 1-(fluorosulfonyl)-2,3-dimethyl-1H-imidazol-3-ium trifluoromethanesulfonate (272 mg, 826.43 mol) at 0° C. and the reaction mixture was stirred at room temperature for 2 h. After completion of the reaction, the reaction mixture was concentrated under reduced pressure and purified by SFC to afford 2-(2,6-dioxo-3-piperidyl)-7-fluorosulfonyloxy-1-oxo-isoquinoline A-93 (41 mg, 20% yield) as a white solid. LC-MS (ES*): m z 355.12 [M+H]+. H-NMR (400 MHz, DMSO-d6): δ 11.10 (s, 1H), 8.27 (d, J=2.4 Hz, 1H), 7.98-7.93 (m, 2H), 7.57 (d, J=7.2 Hz, 1H), 6.81 (d, J=7.6 Hz, 1H), 2.95-2.85 (m, 1H), 2.70-2.60 (m, 2H), 2.10-2.07 (m, 1H) ppm. Preparative SFC Method: Column/dimensions: DCPAK P4VP (30x250) mm,5p; % CO2: 70%; Co solvent: 30% (Methanol); Flow: 100 mL/min; Back Pressure: 100 bar; Temperature: 30° C.; UV: 220 nm; Solubility: MeOH+THF; No. of injections: 15; Total purification time: 02:30 Hr; Run time per injection: 11:00 min Synthesis A91: Synthesis of 5-bromo-2-(2,6-dioxopiperidin-3-yl)-1-oxo-1,2-dihydroisoquinolin-7-yl sulfurofluoridate (A-94): O OO NH₂ CAS #872-36-6 H HN O [CpERhCl2]2 O O CAS #:2353-44-8 O HO OH pAgSbFe, 0 MeOH, then Et₃N 0 OH ANSbF₆, DCE Step 1 HO Step 2 Br Br /CAS #:140472-69-1 Br H AISF O O N O DBU, THF FO₂SO N Step 3 Br A-94

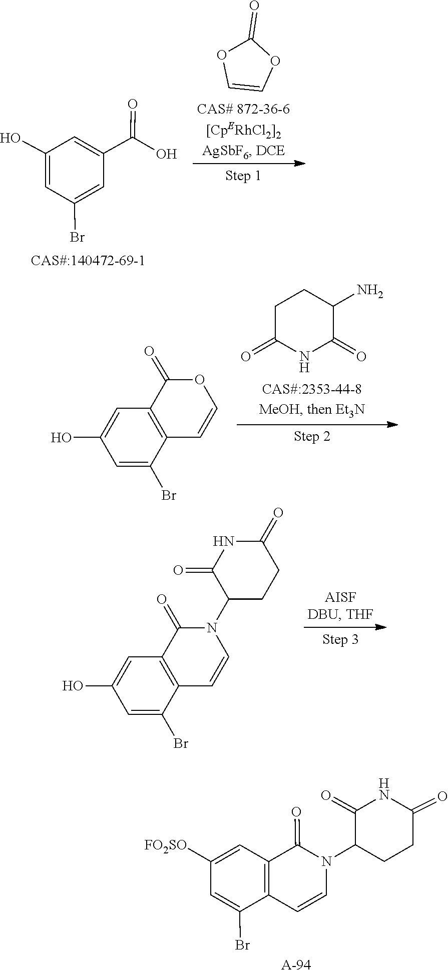

Step 1: 5-bromo-7-hydroxy-1H-isochromen-1-one To a flask of 3-bromo-5-hydroxybenzoic acid (1 eq), [Cp^{E}RhCl₂](0.025 eq) and 10 AgSbF6 (0.15 eq) is added 1,3-dioxol-2-one (CAS #872-36-6, 1 eq) followed by DCE and the reaction is stirred at 120° C. for 15 hours until judged complete. Standard workup and purification provide 5-bromo-7-hydroxy-1H-isochromen-1-one.

Step 2: 3-(5-bromo-7-hydroxy-1-oxoisoquinolin-2(1H)-yl)piperidine-2,6-dione A solution of 5-bromo-7-hydroxy-1H-isochromen-1-one (1.0 eq), 3-aminopiperidine-2,6-dione (CAS #: 2353-44-8, 1.0 eq) MeOH is heated at reflux for 1 hour. To the reaction mixture is added triethylamine (2 eq) and the reaction mixture is heated at reflux until judged complete. A standard workup and standard purification affords 3-(5-bromo-7-hydroxy-1-oxoisoquinolin-2(1H)-yl)piperidine-2,6-dione.

Step 3: 5-bromo-2-(2,6-dioxopiperidin-3-yl)-1-oxo-1,2-dihydroisoquinolin-7-yl 20 sulfurofluoridate (A-94) To a solution of 3-(5-bromo-7-hydroxy-1-oxoisoquinolin-2(1H)-yl)piperidine-2,6-dione (1 eq) in THF at room temperature is added DBU (2 eq) followed by AISF (1.5 eq) and the reaction is stirred until judged complete. Standard workup and purification would provide 5-bromo-2-(2,6-dioxopiperidin-3-yl)-1-oxo-1,2-dihydroisoquinolin-7-yl sulfurofluoridate (A-94).

Synthesis A92: Synthesis of 2-(2,6-dioxopiperidin-3-yl)-1-oxo-1,2-dihydroisoquinoline-7-sulfonyl fluoride (A-95):

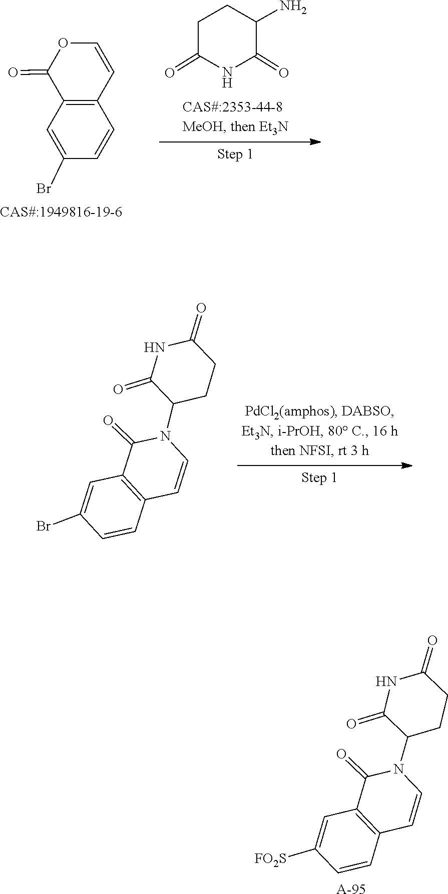

NH₂ O0 oHN HN O O NH H N0 0 C H O PdCl₂(amphos), DABSO, CAS #:2353-44-8 N Et₃N, i-PrOH, 80° C., 16 h N
MeOH, then Et₃NN then NFSI, rt 3 h / Step 1 Step 2 Br Br FO₂S CAS #:1949816-19-6 A-95 Step 1: 3-(7-bromo-1-oxoisoquinolin-2(1H)-yl)piperidine-2,6-dione ^{P}GP-⁷²³,C³ A solution of 7-bromo-1H-isochromen-1-one (CAS #:1949816-19-6, 1.0 eq), 3-aminopiperidine-2,6-dione (CAS #: 2353-44-8, 1.0 eq) MeOH is heated at reflux for 1 hour. To 10 the reaction mixture is added triethylamine (2 eq) and the reaction mixture is heated at reflux until judged complete. A standard workup and standard purification affords 3-(7-bromo-1-oxoisoquinolin-2(1H)-yl)piperidine-2,6-dione.

Step 2: 2-(2,6-dioxopiperidin-3-yl)-1-oxo-1,2-dihydroisoquinoline-7-sulfonylfluoride (A-95) To a solution of 3-(7-bromo-1-oxoisoquinolin-2(1H)-yl)piperidine-2,6-dione (1 eq) and DABSO (0.6 eq) in i-PrOH at room temperature is added triethylamine (3 eq) followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room 20 temperature until judged complete. The reaction is quenched with saturated aqueous sodium thiosulfate and a standard workup is performed, the intermediate is concentrated under reduced pressure and purified under standard conditions to provide 2-(2,6-dioxopiperidin-3-yl)-1-oxo-1,2-dihydroisoquinoline-7-sulfonylfluoride (A-95).

Synthesis A93: Synthesis of 3-(2,6-dioxopiperidin-3-yl)-2-methyl-4-oxo-3,4-dihydroquinazolin-7-yl sulfurofluoridate (A-96):

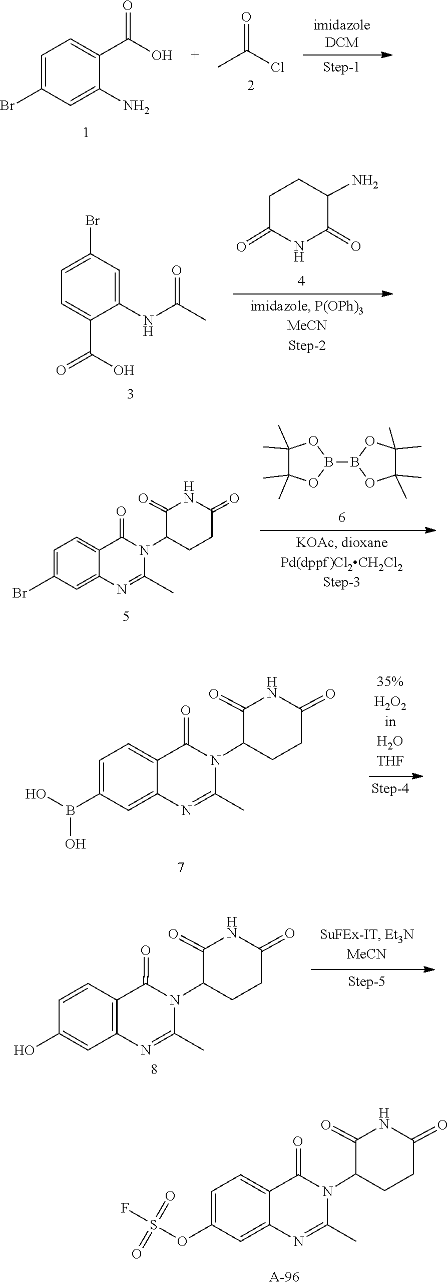

Step-1: To a stirred solution of 2-amino-4-bromo-benzoic acid 1 (3 g, 13.89 mmol) and imidazole (2.84 g, 41.66 mmol) in DCM (30 mL) was added acetyl chloride 2 (1.68 mL, 27.77 mmol) at 0° C. and the reaction mixture was stirred at room temperature for 16 h. 10 After completion of the reaction, the reaction mixture was diluted with water (200 mL) and extracted with ethyl acetate (350 mL×2). The combined organic layer was washed with brine (50 mL), dried over anhydrous sodium sulfate and concentrated under reduced pressure to give the crude product, which was purified by column chromatography (100-200 mesh silica, ~100% ethyl acetate as eluent) to afford 2-acetamido-4-bromo-benzoic acid 3 (2 g, 29% 15 yield) as an off white solid. LC-MS (ES-): m z 257.99 [M - H]-Step-2: To a stirred mixture of 2-acetamido-4-bromo-benzoic acid 3 (2 g, 7.75 mmol) in acetonitrile (35 mL) were added 3-aminopiperidine-2,6-dione 4 (1.02 g, 6.20 mmol, HCl salt), imidazole (1.84 g, 27.05 mmol) and triphenyl phosphite (2.40 g, 7.75 mmol, 5 20 mL) and the reaction mixture was heated to 80° C. for 22 h. After completion of the reaction, the reaction mixture was concentrated under reduced pressure to give the crude product, which was purified by column chromatography (100-200 mesh silica-gel, ~5% methanol in dichloromethane as an eluent) to afford 3-(7-bromo-2-methyl-4-oxo-quinazolin-3-yl)piperidine-2,6-dione 5 (1 g, 35% yield) as an off-white solid. LC-MS (ES-): m z 349.84 [M
H]-Step-3: To a stirred solution of 3-(7-bromo-2-methyl-4-oxo-quinazolin-3-yl)piperidine-2,6-dione 5 (1 g, 2.86 mmol) and 4,4,5,5-tetramethyl-2-(4,4,5,5-tetramethyl-1,3,2-dioxaborolan-2-yl)-1,3,2-dioxaborolane 6 (1.09 g, 4.28 mmol) in 1,4 dioxane (15 mL) was added potassium acetate (841 mg, 8.57 mmol) and degassed with argon gas for 10 min. Then Pd(dppf)Cl2CH2Cl2 (116.61 mg, 0.142 mmol) was added, and the reaction mixture was stirred at 100° C. for 16 h. After completion of the reaction, the reaction mixture was filtered through a pad of celite, which was subsequently washed with ethyl acetate (150 mL). The filtrate was concentrated under reduced pressure to give the crude product, which was purified by column chromatography (100-200 mesh silica, ~100% ethyl acetate as eluent) to afford [3-(2,6-dioxo-3-piperidyl)-2-methyl-4-oxo-quinazolin-7-yl]boronic acid 7 (0.5 g, 46% yield) as a reddish-brown liquid. LC-MS (ES*): m z 316.0 [M+H]+Step-4: To a stirred solution of [3-(2,6-dioxo-3-piperidyl)-2-methyl-4-oxo-quinazolin-7-yl]boronic acid 7 (0.5 g, 1.59 mmol) in tetrahydrofuran (10 mL) was added 35% hydrogen peroxide in water (1 mL, 1.14 mol) at 0° C. The reaction mixture was stirred at room temperature for 1 h. After completion of the reaction, the reaction mixture was quenched with an aq. solution of sodium bisulfite (100 mL) and extracted with ethyl acetate (150 mL×2).

The combined organic layer was dried over sodium sulfate and concentrated under reduced pressure to afford 3-(7-hydroxy-2-methyl-4-oxo-quinazolin-3-yl)piperidine-2,6-dione 8 (0.3 g, 67% yield) as a brown semi-solid. LC-MS (ES+): m z 288.15 [M+H]+Step-5: To a stirred solution of 3-(7-hydroxy-2-methyl-4-oxo-quinazolin-3-yl)piperidine-2,6-dione 8 (0.3 g, 1.04 mmol) in acetonitrile (4 mL) were added 1-(fluorosulfonyl)-2,3-dimethyl-1H-imidazol-3-ium trifluoromethanesulfonate (515.79 mg, 1.57 mmol) and triethylamine (0.291 mL, 2.09 mmol) at room temperature and the reaction mixture was stirred for 1 h. After completion of the reaction, the solvent was removed under reduced pressure to give the crude, which was purified by Prep-HPLC to afford 3-(2,6-dioxo-3-piperidyl)-7-fluorosulfonyloxy-2-methyl-4-oxo-quinazoline A-96 (67 mg, 17% yield) as an off-white solid. LC-MS (ES+): m z 370.23 [M+H]+. H-NMR (400 MHz, DMSO-d6): δ 11.07 (s, 1H), 8.23 (d, J=8.8 Hz, 1H), 7.89 (d, J=2.0 Hz, 1H), 7.71 (dd, J=8.8 Hz, 1H), 5.32 (q, J=5.7 Hz, 1H), 2.89-2.80 (m, 1H), 2.67-2.62 (m, 5H), 2.22-2.18 (m, 1H) ppm. Prep IPLC method: Column/dimensions: Sunfire (19*150*5 p); Mobile phase A: 0.1% formic acid in water; Mobile phase B: 100% acetonitrile; Gradient (Time/% B):0/10,2/10,13/80; Flow rate: 18 mL/min; Solubility: acetonitrile+water+tetrahydrofuran Synthesis A94: Synthesis of 6-bromo-3-(2,6-dioxopiperidin-3-yl)-2-methyl-4-oxo-3,4-dihydroquinazolin-7-ylsulfurofluoridate (A-97):

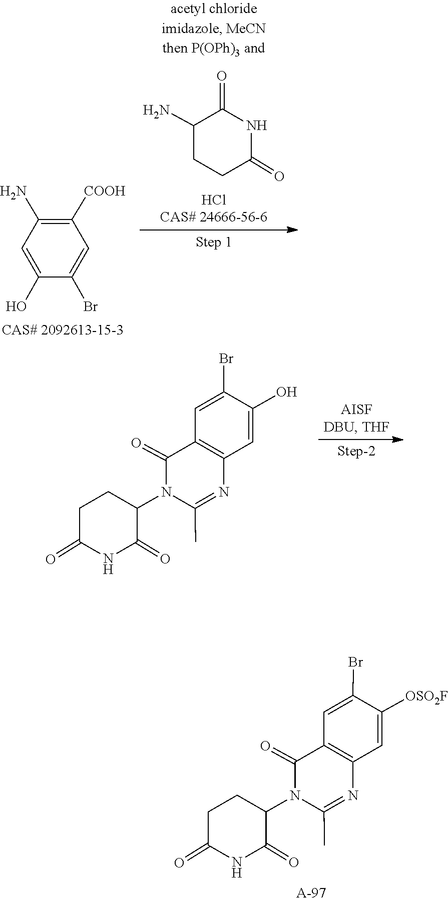

Step 1: To a solution of 2-amino-5-bromo-4-hydroxybenzoic acid (CAS #2092613-15-3, 1.0 eq), imidazole (1.2 eq) in acetonitrile is added acetyl chloride (1.3 mmol) and stirred at room temperature until judged complete. To the reaction mixture is added 3-aminopiperidine-2,6-dione (1 eq), imidazole (2.4 eq) and P(OPh)3 (1.3 eq) and is heated at reflux until judged complete. A standard workup and standard purification affords 3-(6-bromo-7-hydroxy-2-methyl-4-oxoquinazolin-3(4H)-yl)piperidine-2,6-dione.

Step 2: To a solution of 3-(6-bromo-7-hydroxy-2-methyl-4-oxoquinazolin-3(4H)-yl)piperidine-2,6-dione (1 eq) in THF at room temperature is added DBU (2 eq) followed by AISF (1.5 eq) and the reaction is stirred until judged complete. Standard workup and purification would provide 6-bromo-3-(2,6-dioxopiperidin-3-yl)-2-methyl-4-oxo-3,4-dihydroquinazolin-7-yl sulfurofluoridate (A-97).

Synthesis A95: Synthesis of 3-(2,6-dioxopiperidin-3-yl)-2-methyl-4-oxo-3,4-dihydroquinazoline-7-sulfonyl fluoride (A-98):

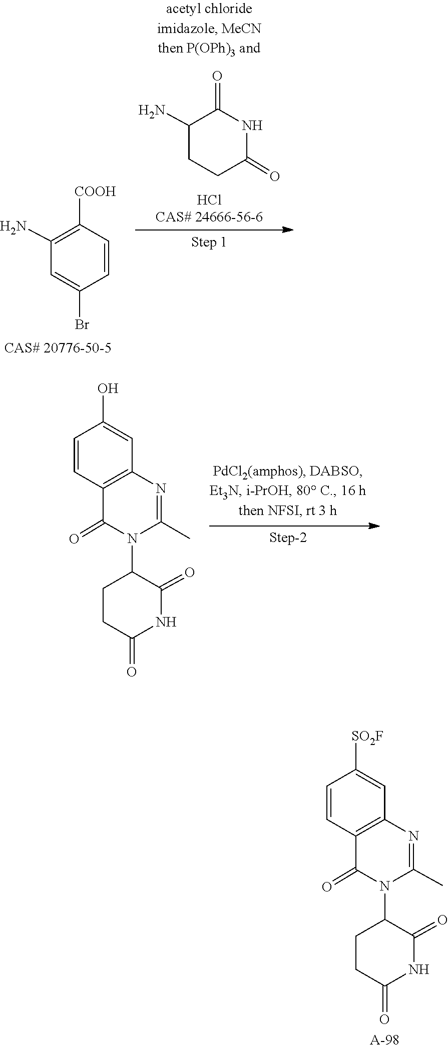

acetyl chloride imidazole, MeCN then P(OPh)₃ and O SO2F H₂N OH NH

I PdCl₂(amphos), DABSO, COOH HCl N Et₃N, i-PrOH, 80° C., 16 h N H2N CAS #24666-56-6 I then NFSI, rt 3 h 0 N Step 1 Ste 2K H₂O tp Step 2 a Br NH NH CAS #20776-50-5 A-98

Step 1: 3-(7-hydroxy-2-methyl-4-oxoquinazolin-3(4H)-yl)piperidine-2,6-dione To a solution of 2-amino-4-bromobenzoic acid (CAS #20776-50-5, 1.0 eq), imidazole (1.2 eq) in acetonitrile is added acetyl chloride (1.3 mmol) and stirred at room temperature until judged complete. To the reaction mixture is added 3-aminopiperidine-2,6-dione, hydrogen chloride (1 eq), imidazole (2.4 eq) and P(OPh)3 (1.3 eq) and is heated at reflux until judged complete. A standard workup and standard purification affords 3-(7-hydroxy-2-methyl-4-oxoquinazolin-3(4H)-yl)piperidine-2,6-dione.

Step 2: 3-(2,6-dioxopiperidin-3-yl)-2-methyl-4-oxo-3,4-dihydroquinazoline-7-sulfonyl fluoride (A-98) To a solution of 3-(7-hydroxy-2-methyl-4-oxoquinazolin-3(4H)-yl)piperidine-2,6-dione (1 eq) and DABSO (0.6 eq) in i-PrOH at room temperature is added triethylamine (3 eq) 15 followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with saturated aqueous sodium thiosulfate and a standard workup is performed, the intermediate is concentrated under reduced 20 pressure and purified under standard conditions to provide 3-(2,6-dioxopiperidin-3-yl)-2-methyl-4-oxo-3,4-dihydroquinazoline-7-sulfonyl fluoride (A-98).

Synthesis A96: Synthesis of 2-(2,6-dioxopiperidin-3-yl)-1-oxo-1,2,3,4-tetrahydroisoquinolin-7-yl sulfurofluoridate (A-99)

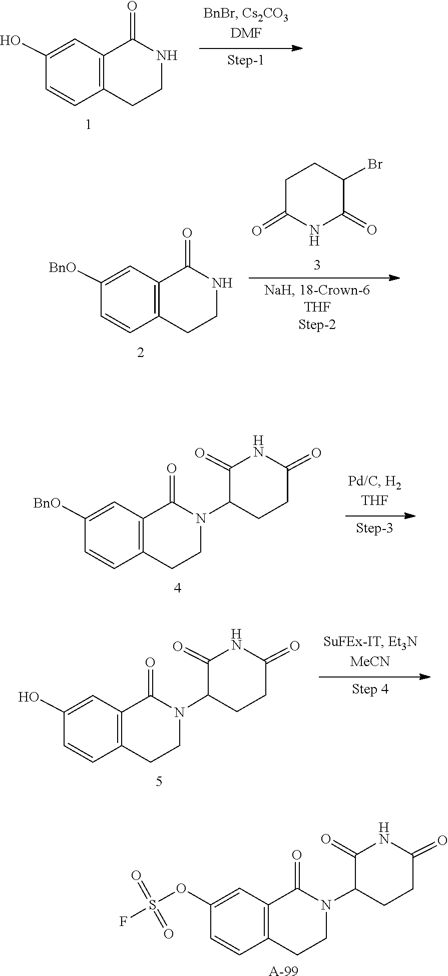

Step-i: To a stirred solution of 7-hydroxy-3,4-dihydro-2H-isoquinolin-1-one 1 (2.0 g, 12.26 mmol) in DMF (10 mL) was added cesium carbonate (3.99 g, 12.26 mmol) followed by benzyl bromide (1.46 mL, 12.26 mmol) at room temperature. The reaction mixture was stirred at room temperature for 16 hours. After completion of the reaction, the reaction mixture was 10 concentrated under reduced pressure. The residue was diluted with ice cold water (30 mL) and extracted with ethyl acetate (30 mL×2). The combined organic layer was dried over anhydrous sodium sulfate and concentrated under reduced pressure to give the crude product, which was purified by column chromatography (Davisil silica with ~48% of ethyl acetate in pet ether as an eluent) to afford 7-benzyloxy-3,4-dihydro-2H-isoquinolin-1-one 2 (2.1 g, 58% yield) as an off-white solid. LC-MS (ES*): m z 254.14 [M+H]+Step-²: To a stirred solution of 7-benzyloxy-3,4-dihydro-2H-isoquinolin-1-one 2 (1.0 g, 3.95 mmol) in THF (100 mL) were added sodium hydride (60% dispersion in mineral oil) (947 mg, 39.48 mmol) and 18-Crown-6 (111 mg, 0.420 mmol) at 0° C. The reaction mixture was stirred at 60° C. for 30 min. Then a solution of 3-bromopiperidine-2,6-dione 3 (3.79 g, 0.019 mmol) in THF (50 mL) was slowly added and stirred at 70° C. for 16 h. After completion of the reaction, the reaction mixture was concentrated under reduced pressure. The residue was diluted with ice cold water (20 mL) and extracted with ethyl acetate (20 mL×2). The combined organic layer was dried over anhydrous sodium sulfate and concentrated under reduced 25 pressure to give the crude product, which was purified by column chromatography (Davisil silica with ~63 00 of ethyl acetate in pet ether as an eluent) to afford 3-(7-benzyloxy-1-oxo-3,4-dihydroisoquinolin-2-yl)piperidine-2,6-dione 4 (0.8 g, 44% yield ) as off-white solid. LC-MS (ES*): m z 365.32 [M+H]+Step-3: To a stirred solution of 3-(7-benzyloxy-1-oxo-3,4-dihydroisoquinolin-2-yl)piperidine-2,6-dione 4 (0.8 g, 2.20 mmol) in THF (15 mL) added 10% palladium on carbon (wetted with ca. 55% water) (0.8 g, 7.52 mmol) and the reaction mixture was stirred at room temperature under hydrogen atmosphere (using a parr shaker) for 2 h. After completion of the reaction, the reaction mixture was filtered through a celite pad using ethyl acetate (30 mL) and the filtrate was concentrated under reduced pressure to afford 3-(7-hydroxy-1-oxo-3,4-dihydroisoquinolin-2-yl)piperidine-2,6-dione 5 (0.6 g, 75% yield ) as an off-white solid. LC-MS (ES+): m z 275.16 [M+H]+Step-4: To a stirred solution of 3-(7-hydroxy-1-oxo-3,4-dihydroisoquinolin-2-yl)piperidine-2,6-dione 5 (150.54 mg, 0.548 mmol) in acetonitrile (10 mL) were added Et3N (0.229 mL, 1.65 mmol ) and 1-(fluorosulfonyl)-2,3-dimethyl-1H-imidazol-3-ium trifluoromethanesulfonate (361 mg, 1.10 mmol) at 0° C. The reaction mixture was stirred at room temperature for 2 h. After completion of the reaction, the reaction mixture was concentrated under reduced pressure to give the crude product, which was purified by Prep-HPLC to afford 2-(2,6-dioxo-3-piperidyl)-7-fluorosulfonyloxy-1-oxo-3,4-dihydroisoquinoline A-99 (92.15 mg, 47% yield) as a white solid. LC-MS (ES*): m z 357.24 [M+H]+. H-NMR (400 MHz, DMSO-d6): δ 10.94 (s, 1H), 7.95 (d, J=2.40 Hz, 1H), 7.78-7.75 (m, 1H), 7.58 (d, J=8.40 Hz, 1H), 5.25 (bs,1H), 3.65-3.50 (m, 2H), 3.15-3.05 (m, 2H), 2.90-2.80 (m, 1H), 2.60-2.55 (m, 1H), 2.40-2.35 (m, 1H), 2.0-1.90 (m, 1H) ppm. Preparative HPLC Method: Column/dimensions: SUNFIRE C18 (19*150*5 um) Mobile phase A: 0.1% FA in water Mobile Phase B: acetonitrile Gradient:0/5, 2/5, 10/30, 10.2/30, 10.3/100, 11/100, 11.01/5, 14/5. Flow rate: 18 mL/min Solubility: THF+water+acetonitrile Synthesis A97: Synthesis of 6-bromo-2-(2,6-dioxopiperidin-3-yl)-1-oxo-1,2,3,4-tetrahydroisoquinolin-7-yl sulfurofluoridate (A-100):

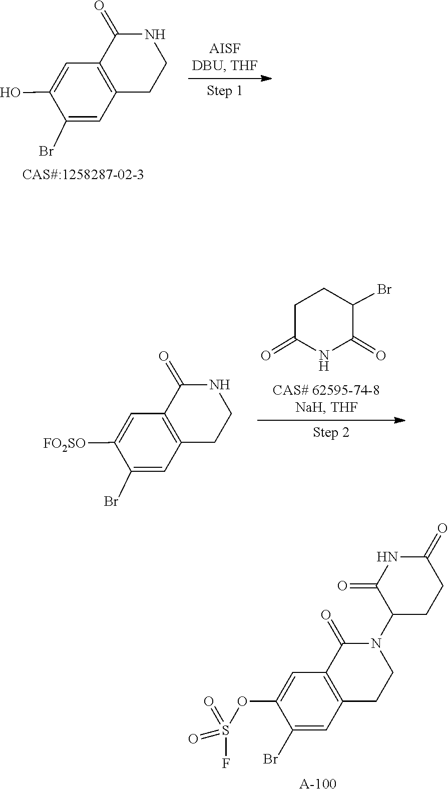

6-bromo-2-(2,6-dioxopiperidin-3-yl)-1-oxo-1,2,3,4-tetrahydroisoquinolin-7-yl^{PGP}-7²⁸,C³ sulfurofluoridate (A-100) can be prepared in a similar manner to A-79, except replacing 6-bromoindolin-4-ol with 6-bromo-7-hydroxy-3,4-dihydroisoquinolin-1(2H)-one (CAS #1258287-02-3).

Synthesis A98: Synthesis of 2-(2,6-dioxopiperidin-3-yl)-1-oxo-1,2,3,4-tetrahydroisoquinoline-7-sulfonyl fluoride (A-101):

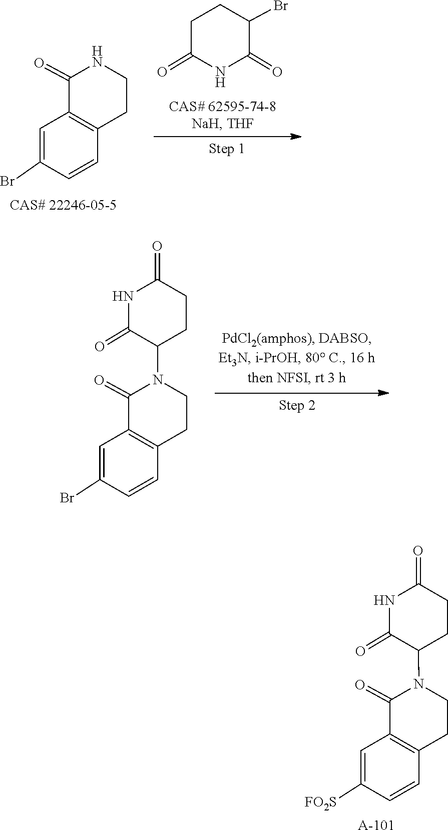

2-(2,6-dioxopiperidin-3-yl)-1-oxo-1,2,3,4-tetrahydroisoquinoline-7-sulfonyl fluoride (A-101) can be prepared in a similar manner to A-80, except replacing 4-bromoindoline with 7-bromo-3,4-dihydroisoquinolin-1(2H)-one (CAS #22246-05-5).

Synthesis A99: Synthesis of 2-(2,4-dioxo-3-azabicyclo[3.1.1]heptan-1-yl)-3-oxoisoindoline-5-sulfonyl fluoride (A-102):

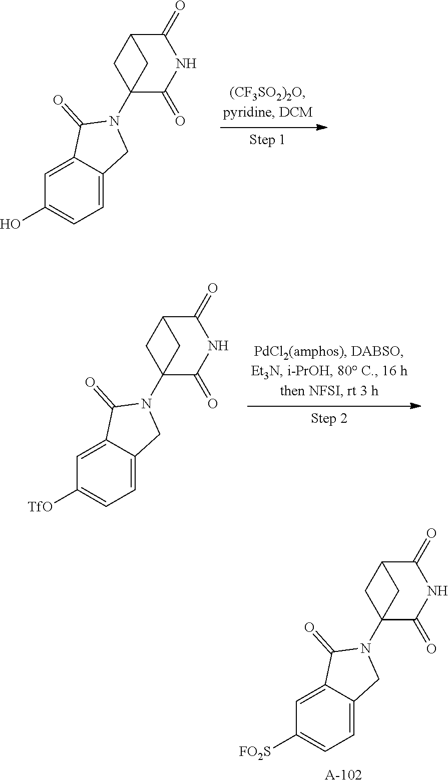

Step 1: 2-(2,4-dioxo-3-azabicyclo[3.1.1]heptan-1-yl)-3-oxoisoindolin-5-yl 5 trifluoromethanesulfonate To a solution of 1-(6-hydroxy-1-oxoisoindolin-2-yl)-3-azabicyclo[3.1.1]heptane-2,4-dione (1 eq) in DCM at 0° C. is added pyridine (1.2 eq) followed by (CF3SO2)20 (1.2 eq) and the reaction is stirred until judged complete. Standard workup and purification 2-(2,4-dioxo-3-azabicyclo[3.1.1]heptan-1-yl)-3-oxoisoindolin-5-yl trifluoromethanesulfonate. 10 Step 2: 2-(2,4-dioxo-3-azabicyclo[3.1.1]heptan-1-yl)-3-oxoisoindoline-5-sulfonyl fluoride (A-102) To a solution of 2-(2,4-dioxo-3-azabicyclo[3.1.1]heptan-1-yl)-3-oxoisoindolin-5-yl trifluoromethanesulfonate (1 eq) and DABSO (0.6 eq) in i-PrOH at room temperature is added triethylamine (3 eq) followed by purging with N2 for 5 minutes. Pd(amphos)C12 (0.05 eq) is 15 added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with saturated aqueous sodium thiosulfate and a standard workup is performed, the intermediate is concentrated under reduced pressure and purified under standard conditions to provide 2-(2,4-dioxo-3-azabicyclo[3.1.1]heptan-1-yl)-3-oxoisoindoline-5-sulfonyl fluoride (A-102). Synthesis A100: Synthesis of 2-(2,4-dioxo-3-azabicyclo[3.1.1]heptan-1-yl)-1,3-dioxoisoindolin-5-ylsulfurofluoridate (A-103):

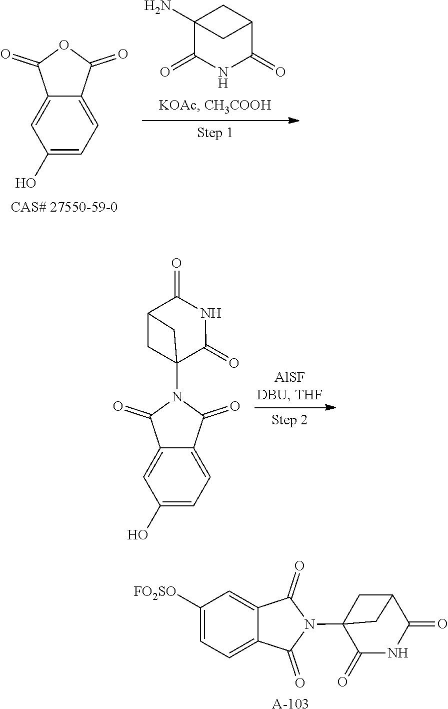

Step 1: 1-(4-bromo-6-hydroxy-1-oxoisoindolin-2-yl)-3-azabicyclo[3.1.1]heptane-2,4-dione To a solution of 5-hydroxyisobenzofuran-1,3-dione (CAS #27550-59-0, 1 eq), 1-amino-3-azabicyclo[3.1.1]heptane-2,4-dione (CAS #2330879-78-0, 1.2 eq) and potassium acetate (2.5 eq) in acetic acid is stirred at 120° C. overnight or until judged complete. Standard workup and purification provide 1-(4-bromo-6-hydroxy-1-oxoisoindolin-2-yl)-3-azabicyclo[3.1.1]heptane-2,4-dione.

Step 2: 2-(2,4-dioxo-3-azabicyclo[3.1.1]heptan-1-yl)-1,3-dioxoisoindolin-5-ylsulfurofluoridate (A-103) To a solution of 1-(4-bromo-6-hydroxy-1-oxoisoindolin-2-yl)-3-azabicyclo[3.1.1]heptane-2,4-dione (1 eq) in THF at room temperature is added DBU (2 eq) 15 followed by AISF (1.5 eq) and the reaction is stirred until judged complete. Standard workup and purification provide 2-(2,4-dioxo-3-azabicyclo[3.1.1]heptan-1-yl)-1,3-dioxoisoindolin-5-ylsulfurofluoridate (A-103).

Synthesis A101: Synthesis of 7-bromo-2-(2,4-dioxo-3-azabicyclo[3.1.1]heptan-1-yl)-1,3-dioxoisoindolin-5-yl sulfurofluoridate (A-104):

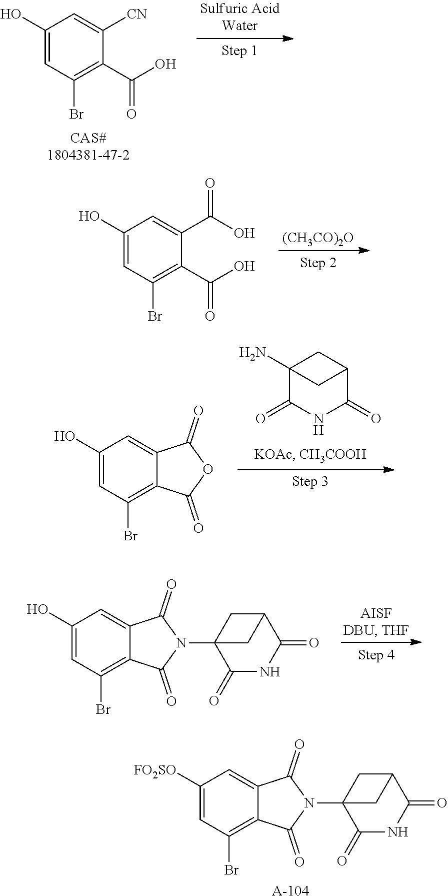

Step 1: 3-bromo-5-hydroxyphthalic acid To a solution of 2-bromo-6-cyano-4-hydroxybenzoic acid (CAS #1804381-47-2, 1 eq) in sulfuric acid (⁸0% in water, 3 eq) is stirred and heated at 150° C. overnight or until judged 5 complete. The reaction is diluted with ice-chilled water and extracted with ethyl acetate. A standard purification provides 3-bromo-5-hydroxyphthalic acid.

Step 2: 4-bromo-6-hydroxyisobenzofuran-1,3-dione A solution of 3-bromo-5-hydroxyphthalic acid (1 eq) in acetic anhydride (0.6 M) is stirred and heated at reflux until judged complete. The reaction is concentrated under reduced 10 pressure to provide the crude product. A standard purification is performed to provide 4-bromo-6-hydroxyisobenzofuran-1,3-dione.

Step 3: 1-(4-bromo-6-hydroxy-1,3-dioxoisoindolin-2-yl)-3-azabicyclo[3.1.1]heptane-2,4-dione To a solution of 4-bromo-6-hydroxyisobenzofuran-1,3-dione (1 eq), 1-amino-3-azabicyclo[3.1.1]heptane-2,4-dione (CAS #2330879-78-0, 1.2 eq) and potassium acetate (2.5 eq) in acetic acid is stirred at 120° C. overnight or until judged complete. Standard workup and purification 1-(4-bromo-6-hydroxy-1,3-dioxoisoindolin-2-yl)-3-azabicyclo[3.1.1]heptane-2,4-dione.

Step 4: 7-bromo-2-(2,4-dioxo-3-azabicyclo[3.1.1]heptan-1-yl)-1,3-dioxoisoindolin-5-yl 20 sulfurofluoridate (A-104) To a solution of 1-(4-bromo-6-hydroxy-1,3-dioxoisoindolin-2-yl)-3-azabicyclo[3.1.1]heptane-2,4-dione (1 eq) in THF at room temperature is added DBU (2 eq) followed by AISF (1.5 eq) and the reaction is stirred until judged complete. Standard workup and purification provide 7-bromo-2-(2,4-dioxo-3-azabicyclo[3.1.1]heptan-1-yl)-1,3-dioxoisoindolin-5-yl sulfurofluoridate A-104.

Synthesis A102: Synthesis of 2-(2,4-dioxo-3-azabicyclo[3.1.1]heptan-1-yl)-1,3-dioxoisoindoline-5-sulfonyl fluoride (A-105):

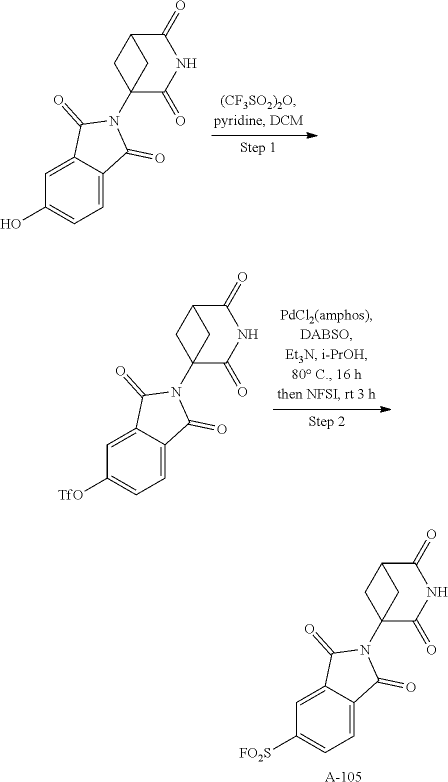

Step 1: 2-(2,4-dioxo-3-azabicyclo[3.1.1]heptan-1-yl)-1,3-dioxoisoindolin-5-yl 5 trifluoromethanesulfonate To a solution of 1-(5-hydroxy-1,3-dioxoisoindolin-2-yl)-3-azabicyclo[3.1.1]heptane-2,4-dione (1 eq) in DCM at 0° C. is added pyridine (1.2 eq) followed by (CF3SO2)20 (1.2 eq) and the reaction is stirred until judged complete. Standard workup and purification 2-(2,4-dioxo-3-azabicyclo[3.1.1]heptan-1-yl)-1,3-dioxoisoindolin-5-yl trifluoromethanesulfonate.

Step 2: 2-(2,4-dioxo-3-azabicyclo[3.1.1]heptan-1-yl)-1,3-dioxoisoindoline-5-sulfonyl fluoride (A-105) To a solution of 2-(2,4-dioxo-3-azabicyclo[3.1.1]heptan-1-yl)-1,3-dioxoisoindolin-5-yl trifluoromethanesulfonate (1 eq) and DABSO (0.6 eq) in i-PrOH at room temperature is added triethylamine (3 eq) followed by purging with N2 for 5 minutes. Pd(amphos)C12 (0.05 eq) is 15 added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with sat. aq. sodium thiosulfate and a standard workup is performed, the intermediate is concentrated under reduced pressure and purified under standard conditions to provide 2-(2,4-dioxo-3-azabicyclo[3.1.1]heptan-1-yl)-1,3-dioxoisoindoline-5-sulfonyl fluoride (A-105). Synthesis A103: Synthesis of 3-(2,6-dioxopiperidin-3-yl)-1-methyl-1H-indol-6-yl sulfurofluoridate (A-106):

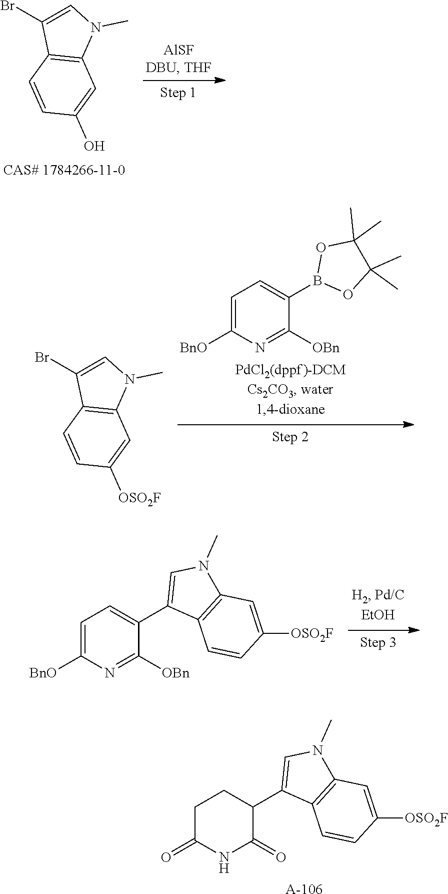

5 Step 1: 3-bromo-1-methyl-1H-indol-6-yl sulfurofluoridate To a solution of 3-bromo-1-methyl-1H-indol-6-ol (CAS #1784266-11-0, 1 eq) in THF at room temperature is added DBU (2 eq) followed by AISF (1.5 eq) and the reaction is stirred until judged complete. Standard workup and purification provide 3-bromo-1-methyl-1H-indol-6-yl sulfurofluoridate. 10 Step 2: 3-bromo-1-methyl-1H-indol-6-yl sulfurofluoridate To a solution of 3-(2,6-bis(benzyloxy)pyridin-3-yl)-1-methyl-1H-indol-6-yl sulfurofluoridate (1 eq), 2,6-dibenzyloxy-3-(4,4,5,5-tetramethyl-1,3,2-dioxaborolan-2-yl)pyridine (1 eq) in 1,4-dioxane and water was added Cs2CO3 (3 eq) and degassed for 10 minutes using N2. Pd(dppf)C12 DCM (0.10 equiv) is added to the reaction mixture at room 15 temperature and again the reaction is purged for 5 minutes using N2. The reaction mixture is stirred at 90° C. until judged complete. The reaction mixture is filtered through a celite bed and washed with ethyl acetate. The filtrate is concentrated under reduced pressure to afford the crude product and is subjected to a standard purification to afford 3-bromo-1-methyl-1H-indol-6-yl sulfurofluoridate. 20 Step 3: 3-(2,6-dioxopiperidin-3-yl)-1-methyl-1H-indol-6-yl sulfurofluoridate (A-106) To a flask is added 3-bromo-1-methyl-1H-indol-6-yl sulfurofluoridate (1 eq), in ethanol at room temperature is added 10% palladium on carbon (3 eq), and the reaction mixture is stirred at room temperature under H2 atmosphere until the reaction is judged complete. The reaction mixture is filtered through diatomaceous earth, concentrated under reduced pressure and subjected to standard purification to provide 3-(2,6-dioxopiperidin-3-yl)-1-methyl-iH-indol-6-yl sulfurofluoridate A-106.

Synthesis A104: Synthesis of 3-(2,6-dioxopiperidin-3-yl)-1-methyl-1H-indole-6-sulfonyl fluoride (A-107):

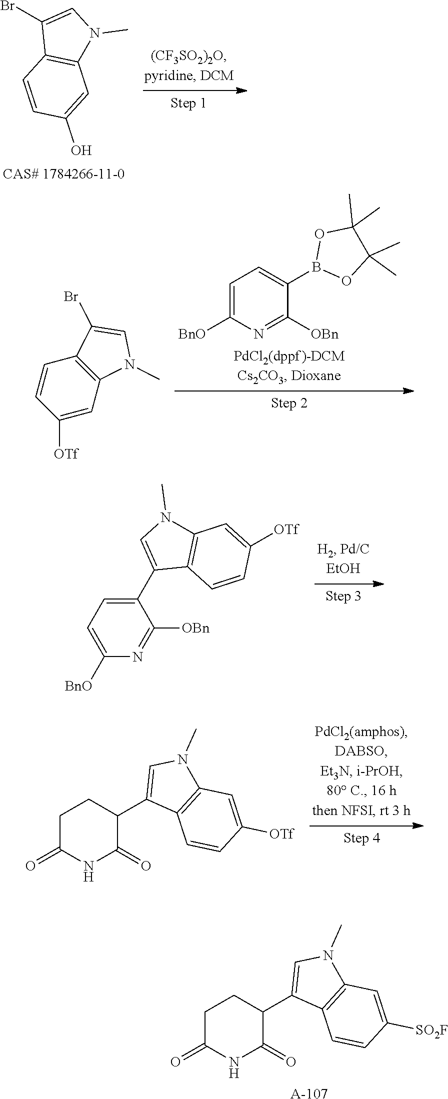

Step 1: 3-bromo-1-methyl-1H-indol-6-yl trifluoromethanesulfonate ^{PGP}-7³⁵,c³ To a solution of 3-bromo-1-methyl-1H-indol-6-ol (1784266-11-0, 1 eq) in DCM at 0° C. is added pyridine (1.2 eq) followed by (CF3SO2)20 (1.2 eq) and the reaction is stirred until judged complete. Standard workup and purification 3-bromo-1-methyl-iH-indol-6-yl trifluoromethanesulfonate.

Step 2: 3-(2,6-bis(benzyloxy)pyridin-3-yl)-1-methyl-1H-indol-6-yl 15 trifluoromethanesulfonate To a solution of 3-bromo-1-methyl-1H-indol-6-yl trifluoromethanesulfonate (1 eq), 2,6-dibenzyloxy-3-(4,4,5,5-tetramethyl-1,3,2-dioxaborolan-2-yl)pyridine (1 eq) in 1,4-dioxane and water was added Cs2CO3 (3 eq) and degassed for 10 minutes using N2. Pd(dppf)Cl2 DCM (0.10 eq) is added to the reaction mixture at room temperature and again the reaction is purged 20 for 5 minutes using N2. The reaction mixture is stirred at 90° C. until judged complete. The reaction mixture is filtered through a celite bed and washed with ethyl acetate. The filtrate is concentrated under reduced pressure to afford the crude product and is subjected to a standard purification to afford 3-(2,6-bis(benzyloxy)pyridin-3-yl)-1-methyl-iH-indol-6-yl trifluoromethanesulfonate.

Step 3: 3-(2,6-dioxopiperidin-3-yl)-1-methyl-1H-indol-6-yl trifluoromethanesulfonate To a flask is added 3-(2,6-bis(benzyloxy)pyridin-3-yl)-1-methyl-1H-indol-6-yl trifluoromethanesulfonate (1 eq), in ethanol at room temperature is added 10% palladium on carbon (3 eq), and the reaction mixture is stirred at room temperature under H2 atmosphere until the reaction is judged complete. The reaction mixture is filtered through diatomaceous earth, concentrated under reduced pressure and subjected to standard purification to provide 3-(2,6-dioxopiperidin-3-yl)-1-methyl-iH-indol-6-yl trifluoromethanesulfonate. Step 3: 3-(2,6-dioxopiperidin-3-yl)-1-methyl-1H-indole-6-sulfonyl fluoride (A-107) To a solution of 3-(2,6-dioxopiperidin-3-yl)-1-methyl-1H-indol-6-yl trifluoromethanesulfonate (1 eq) and DABSO (0.6 eq) in i-PrOH at room temperature is added triethylamine (3 eq) followed by purging with N2 for 5 minutes. Pd(amphos)C12 (0.05 eq) is added and the reaction mixture is heated to 80° C. until judged complete. The reaction is cooled to room temperature and N-fluorobenzenesulfonimide (2 eq) is added, and the reaction mixture is stirred at room temperature until judged complete. The reaction is quenched with saturated aqueous sodium thiosulfate and a standard workup is performed, the intermediate is concentrated under reduced pressure and purified under standard conditions to provide 3-(2,6-dioxopiperidin-3-yl)-1-methyl-1H-indole-6-sulfonyl fluoride (A-107).

Synthesis A105: Synthesis of 3-(2,6-dioxopiperidin-3-yl)isoquinolin-7-yl sulfurofluoridate (A-108):

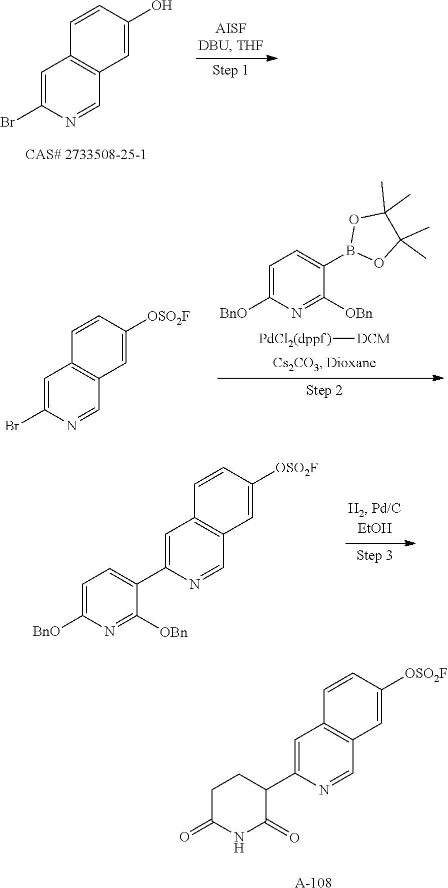

3-(2,6-dioxopiperidin-3-yl)isoquinolin-7-yl sulfurofluoridate (A-108) can be prepared in a similar manner to A-106, except replacing 3-bromo-1-methyl-1H-indol-6-ol with 3-bromoisoquinolin-7-ol (CAS #2733508-25-1).

Synthesis A106: Synthesis of 3-(2,6-dioxopiperidin-3-yl)isoquinoline-7-sulfonyl fluoride (A-109):

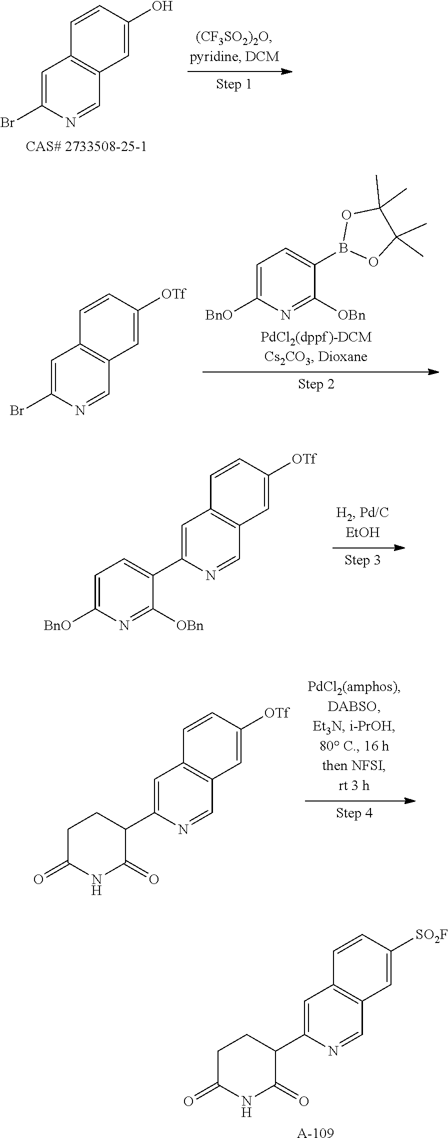

3-(2,6-dioxopiperidin-3-yl)isoquinoline-7-sulfonyl fluoride (A-109) can be prepared in a similar manner to A-107, except replacing 3-bromo-1-methyl-1H-indol-6-ol with 3-bromoisoquinolin-7-ol (CAS #2733508-25-1).

Synthesis A107: Synthesis of 4-(2,6-dioxopiperidin-3-yl)-3,4-dihydro-2H-benzo[b][1,4]oxazin-6-yl sulfurofluoridate (A-110):

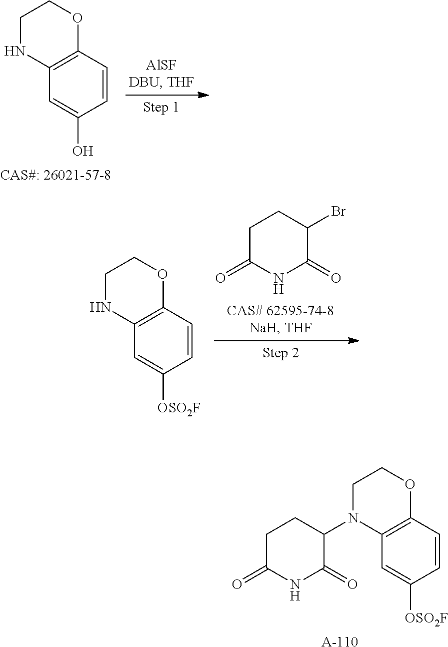

Synthesis A108: Synthesis of 7-bromo-4-(2,6-dioxopiperidin-3-yl)-3,4-dihydro-2H-benzo[b][1,4]oxazin-6-yl sulfurofluoridate (A-111):

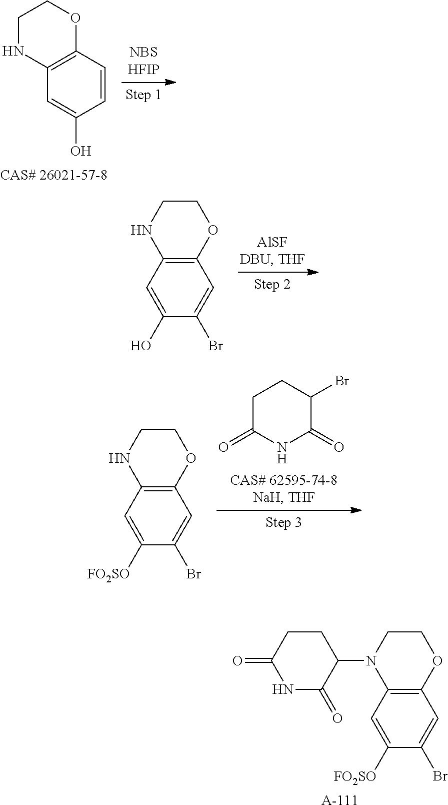

Step 1: 7-bromo-3,4-dihydro-2H-benzo[b][1,4]oxazin-6-ol To a solution of 3,4-dihydro-2H-benzo[b][1,4]oxazin-6-ol (CAS #26021-57-8, 1 eq) in Hexafluoroisopropanol at 0° C. is added N-bromosuccinimide (1.5 eq) and the reaction is stirred at 0° C. until judged complete. Standard workup and purification provide 7-bromo-3,4-dihydro-2H-benzo[b][1,4]oxazin-6-ol.

Step 2: 7-bromo-3,4-dihydro-2H-benzo[b][1,4]oxazin-6-yl sulfurofluoridate To a solution of 7-bromo-3,4-dihydro-2H-benzo[b][1,4]oxazin-6-ol (1 eq) in THF at room temperature is added DBU (2 eq) followed by AISF (1.5 eq) and the reaction is stirred until judged complete. A standard workup and purification provides 7-bromo-3,4-dihydro-2H-benzo[b][1,4]oxazin-6-yl sulfurofluoridate.

Step 3: 7-bromo-4-(2,6-dioxopiperidin-3-yl)-3,4-dihydro-2H-benzo[b][1,4]oxazin-6-yl sulfurofluoridate (A-111) To a solution of sodium hydride (1.1 eq) in THF at 0° C. is added 7-bromo-3,4-dihydro-2H-benzo[b][1,4]oxazin-6-yl sulfurofluoridate (1 eq) and the reaction mixture is stirred at 60 5° C. until judged complete. The reaction mixture is cooled to room temperature and 3-bromopiperidine-2,6-dione (CAS #62595-74-8, 1.2 eq) is added and heated to 60° C. until judged complete. The reaction is quenched with a 20% IPA-DCM solution and washed with water and brine. The organic layer is dried and concentrated under reduced pressure to afford the crude material. Standard purification provides 7-bromo-4-(2,6-dioxopiperidin-3-yl)-3,4-dihydro-2H-benzo[b][1,4]oxazin-6-yl sulfurofluoridate (A-111).

Synthesis A109: Synthesis of 4-(2,6-dioxopiperidin-3-yl)-3,4-dihydro-2H-benzo[b][1,4]oxazine-6-sulfonyl fluoride (A-112):

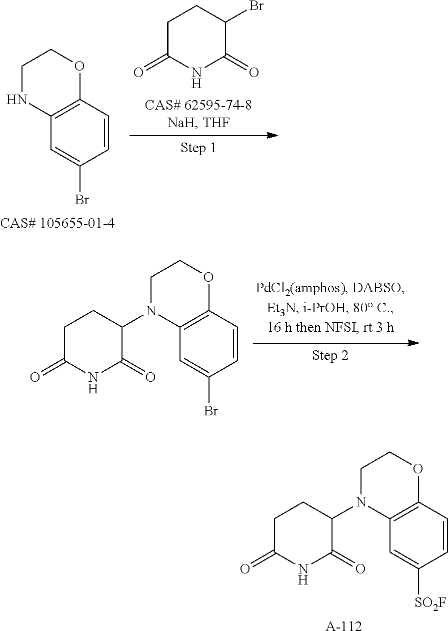 [SMILES: Brc1ccc2c(c1)NCCO2 ; O=C1CCC(Br)C(=O)N1 ; O=C1CCC(N2CCOc3ccc(Br)cc32)C(=O)N1 ; O=C1CCC(N2CCOc3ccc(S(=O)(=O)F)cc32)C(=O)N1 ; "A-112"]

4-(2,6-dioxopiperidin-3-yl)-3,4-dihydro-2H-benzo[b][1,4]oxazine-6-sulfonyl fluoride (A-112) can be prepared in a similar manner to A-80, except replacing 4-bromoindoline with 6-bromo-3,4-dihydro-2H-benzo[b][1,4]oxazine (CAS #105655-01-4).

Synthesis A110: Synthesis of 1-(2,6-dioxopiperidin-3-yl)-1,2,3,4-tetrahydroquinolin-7-yl 20 sulfurofluoridate (A-113):

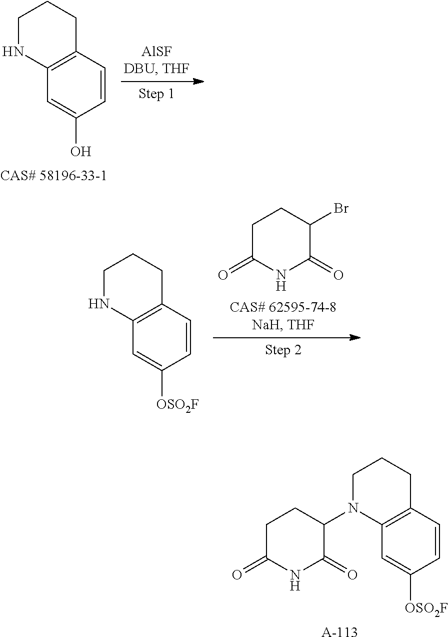

1-(2,6-dioxopiperidin-3-yl)-1,2,3,4-tetrahydroquinolin-7-yl sulfurofluoridate (A-113) can be prepared in a similar manner to A-79, except replacing 6-bromoindolin-4-ol with 1,2,3,4-tetrahydroquinolin-7-ol (CAS #58196-33-1).

Synthesis Al11: Synthesis of 6-bromo-1-(2,6-dioxopiperidin-3-yl)-1,2,3,4-tetrahydroquinolin-7-yl sulfurofluoridate (A-114)

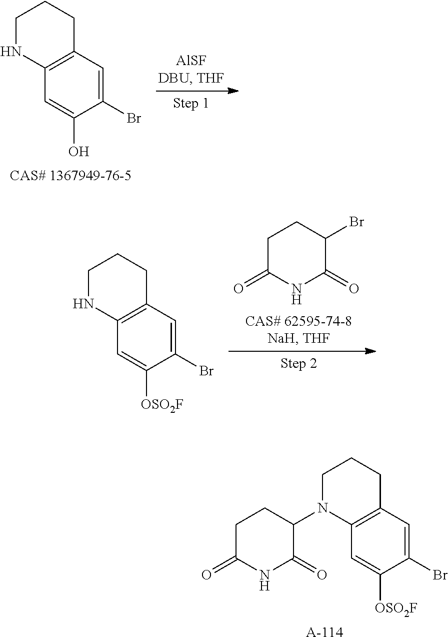

6-bromo-1-(2,6-dioxopiperidin-3-yl)-1,2,3,4-tetrahydroquinolin-7-yl sulfurofluoridate (A-114) can be prepared in a similar manner to A-79, except replacing 6-bromoindolin-4-ol 10 with 6-bromo-1,2,3,4-tetrahydroquinolin-7-ol (CAS #1367949-76-5).

Synthesis A112: Synthesis of 1-(2,6-dioxopiperidin-3-yl)-1,2,3,4-tetrahydroquinoline-7-sulfonyl fluoride (A-115):

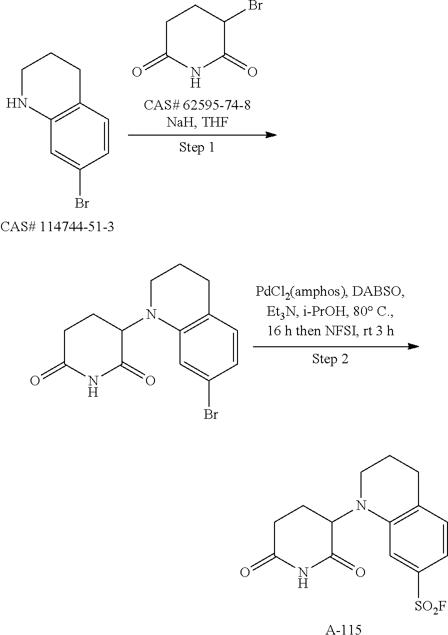 [SMILES: Brc1ccc2c(c1)NCCC2 ; O=C1CCC(Br)C(=O)N1 ; O=C1CCC(N2CCCc3ccc(Br)cc32)C(=O)N1 ; O=C1CCC(N2CCCc3ccc(S(=O)(=O)F)cc32)C(=O)N1 ; "A-115"]

1-(2,6-dioxopiperidin-3-yl)-1,2,3,4-tetrahydroquinoline-7-sulfonyl fluoride (A-115) can be prepared in a similar manner to A-80, except replacing 4-bromoindoline with 7-bromo-1,2,3,4-tetrahydroquinoline (CAS #114744-51-3).

Synthesis A113: Synthesis of 1-(2,6-dioxopiperidin-3-yl)-3-methyl-2-oxo-5-(piperidin-4-yl)-2,3-dihydro-1H-benzo[d]imidazole-4-sulfonyl fluoride (A-116):

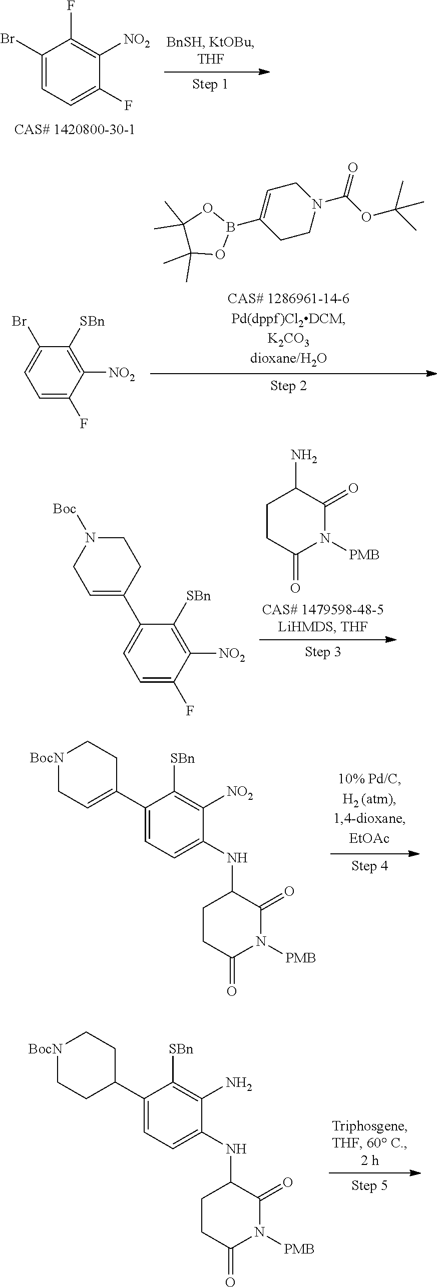

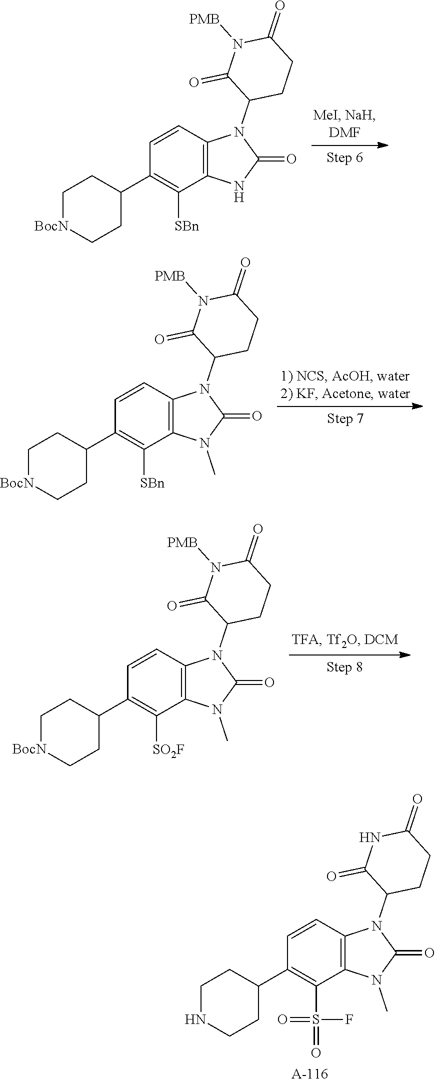

Step 1: Benzyl(6-bromo-3-fluoro-2-nitrophenyl)sulfane To a solution of 1-bromo-2,4-difluoro-3-nitrobenzene (CAS #1420800-30-1, 1 eq) in THF at room temperature is added BnSH (2 eq) followed by KOt-Bu (1.5 eq) and the reaction is stirred at reflux until judged complete. A standard workup and purification provides benzyl(6-bromo-3-fluoro-2-nitrophenyl)sulfane.

Step 2: tert-butyl 4-(2-(benzylthio)-4-fluoro-3-nitrophenyl)-3,6-dihydropyridine-1(2H)-carboxylate To a stirred solution of benzyl(6-bromo-3-fluoro-2-nitrophenyl)sulfane (1 eq) and tert-butyl-4-(4,4,5,5-tetramethyl-1,3,2-dioxaborolan-2-yl)-3,6-dihydro-2H-pyridine-1-carboxylate (1.2 eq) in 1,4-dioxane and water is added potassium carbonate (2 eq) and degassed with argon gas for 10 min. Pd (dppf) C120CH2CI2 (0.05 eq) is added to the reaction and stirred at 80° C. until judged complete. A standard workup and purification provide tert-butyl 4-(2-(benzylthio)-4-fluoro-3-nitrophenyl)-3,6-dihydropyridine-1(2H)-carboxylate.

Step 3: tert-butyl 4-(2-(benzylthio)-4-((1-(4-methoxybenzyl)-2,6-dioxopiperidin-3-yl)amino)-3-nitrophenyl)-3,6-dihydropyridine-1(2H)-carboxylate To a stirred solution of tert-butyl 4-(2-(benzylthio)-4-fluoro-3-nitrophenyl)-3,6-dihydropyridine-1(2H)-carboxylate (1 eq) in THF is added LiHMDS (1.2 eq) stirred at −78° C. 15 for 1 h. 3-amino-1-(4-methoxybenzyl)piperidine-2,6-dione (1 eq) is added and the reaction mixture is stirred at room temperature until judged complete. A standard workup and purification provides tert-butyl 4-(2-(benzylthio)-4-((1-(4-methoxybenzyl)-2,6-dioxopiperidin-3-yl)amino)-3-nitrophenyl)-3,6-dihydropyridine-1(2H)-carboxylate.

Step 4: tert-butyl 4-(3-amino-2-(benzylthio)-4-((1-(4-methoxybenzyl)-2,6-dioxopiperidin-3-yl)amino)phenyl)piperidine-1-carboxylate To a flask is added tert-butyl 4-(2-(benzylthio)-4-((1-(4-methoxybenzyl)-2,6-dioxopiperidin-3-yl)amino)-3-nitrophenyl)-3,6-dihydropyridine-1(2H)-carboxylate in ethanol at room temperature is added 10% palladium on carbon (3 eq), and the reaction mixture is stirred at room temperature under H2 atmosphere until judged complete. The reaction mixture is filtered through diatomaceous earth, concentrated under reduced pressure and subjected to standard purification to provide tert-butyl4-(3-amino-2-(benzylthio)-4-((1-(4-methoxybenzyl)-2,6-dioxopiperidin-3-yl)amino)phenyl)piperidine-1-carboxylate.

Step 5: tert-butyl 4-(4-(benzylthio)-1-(1-(4-methoxybenzyl)-2,6-dioxopiperidin-3-yl)-2-oxo-2,3-dihydro-1H-benzo[d]imidazol-5-yl)piperidine-1-carboxylate To a stirred solution of tert-butyl4-(3-amino-2-(benzylthio)-4-((1-(4-methoxybenzyl)-2,6-dioxopiperidin-3-yl)amino)phenyl)piperidine-1-carboxylate (1 eq) in THF is added triphosgene (1 eq) and stirred at 60° C. until judged complete. A standard workup and purification provides tert-butyl 4-(4-(benzylthio)-1-(1-(4-methoxybenzyl)-2,6-dioxopiperidin-3-yl)-2-oxo-2,3-dihydro-1H-benzo[d]imidazol-5-yl)piperidine-1-carboxylate. Step 6: tert-butyl 4-(4-(benzylthio)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate To a stirred solution of tert-butyl 4-(4-(benzylthio)-1-(1-(4-methoxybenzyl)-2,6-dioxopiperidin-3-yl)-2-oxo-2,3-dihydro-1H-benzo[d]imidazol-5-yl)piperidine-1-carboxylate (1 eq) in DMF is added NaH (2 eq) at 0° C. and stirred for 10 minutes. Methyl iodide (113 pL, 1.81 mmol) is added and the reaction is stirred at room temperature until judged complete. A standard workup and purification provides tert-butyl 4-(4-(benzylthio)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate. 10 Step 7: tert-butyl 4-(4-(fluorosulfonyl)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate To a solution of tert-butyl 4-(4-(benzylthio)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate (1 eq) in acetic acid and water is added N-chlorosuccinimide (2 eq) at room 15 temperature and stirred until complete. A standard workup and purification provides tert-butyl 4-(4-(chlorosulfonyl)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate. This material is taken forward crude. To a stirred solution of tert-butyl 4-(4-(chlorosulfonyl)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate (1 eq) in acetone and water, is added potassium fluoride (1 eq) and stirred at room temperature until judged complete. A standard workup and purification provides tert-butyl 4-(4-(fluorosulfonyl)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate.

Step 7: 1-(2,6-dioxopiperidin-3-yl)-3-methyl-2-oxo-5-(piperidin-4-yl)-2,3-dihydro-1H-benzo[d]imidazole-4-sulfonyl fluoride (A-116) To a stirred solution of tert-butyl 4-(4-(fluorosulfonyl)-1-(1-(4-methoxybenzyl)-2,6-dioxopiperidin-3-yl)-3-methyl-2-oxo-2,3-dihydro-1H-benzo[d]imidazol-5-yl)piperidine-1-carboxylate (1 eq) in DCM is added TFA (1.2 eq) and triflic acid (2 eq) at 0° C. and stirred at room temperature until judged complete. A standard workup and purification provide 1-(2,6-dioxopiperidin-3-yl)-3-methyl-2-oxo-5-(piperidin-4-yl)-2,3-dihydro-1H-benzo[d]imidazole-4-sulfonyl fluoride (A-116).

Synthesis A114: Synthesis of 3-(2,6-dioxopiperidin-3-yl)-2-oxo-6-(piperidin-4-yl)-2,3-dihydrobenzo[d]oxazole-7-sulfonyl fluoride (A-117):

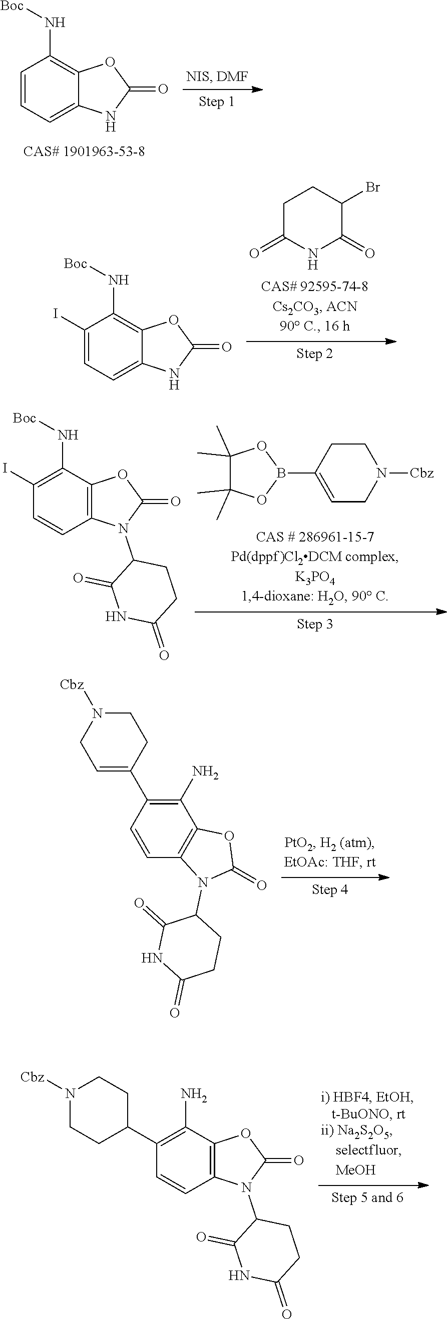

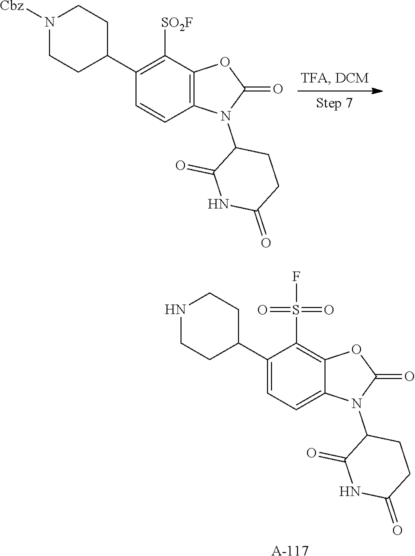

Step 1: tert-butyl (6-iodo-2-oxo-2,3-dihydrobenzo[d]oxazol-7-yl)carbamate To a solution of tert-butyl (2-oxo-2,3-dihydrobenzo[d]oxazol-7-yl)carbamate (CAS #1901963-53-8, 1 eq) in DMF is added NIS (2 eq) at 0° C. The reaction mixture is heated at 100° C. until judged complete. A standard workup and purification provides tert-butyl (6-iodo-2-oxo-2,3-dihydrobenzo[d]oxazol-7-yl)carbamate.

Step 2: tert-butyl (3-(2,6-dioxopiperidin-3-yl)-6-iodo-2-oxo-2,3-dihydrobenzo[d]oxazol-7-yl)carbamate To a stirred solution of tert-butyl (6-iodo-2-oxo-2,3-dihydrobenzo[d]oxazol-7-yl)carbamate (1 eq) in acetonitrile were added Cesium carbonate (2 eq), 3-bromopiperidine-2,6-dione 2 (CAS #62595-74-8, 2eq) and stirred at 80° C. until judged complete. A standard workup and purification provides tert-butyl (3-(2,6-dioxopiperidin-3-yl)-6-iodo-2-oxo-2,3-dihydrobenzo[d]oxazol-7-yl)carbamate.

Step 3: Benzyl 4-(7-amino-3-(2,6-dioxopiperidin-3-yl)-2-oxo-2,3-dihydrobenzo[d]oxazol-6-yl)-3,6-dihydropyridine-1(2H)-carboxylate To a stirred solution of tert-butyl (3-(2,6-dioxopiperidin-3-yl)-6-iodo-2-oxo-2,3-dihydrobenzo[d]oxazol-7-yl)carbamate (1 eq) and tert-butyl-4-(4,4,5,5-tetramethyl-1,3,2-dioxaborolan-2-yl)-3,6-dihydro-2H-pyridine-1-carboxylate (CAS #286961-15-7, 1.2 eq) in 1,4-dioxane and water is added potassium phosphate tribasic (2 eq) and degassed with argon 10 gas for 10 minutes. Pd (dppf) C12 DCM (0.05 eq) is added to the reaction and stirred at 80° C. until judged complete. A standard workup and purification provide benzyl 4-(7-amino-3-(2,6-dioxopiperidin-3-yl)-2-oxo-2,3-dihydrobenzo[d]oxazol-6-yl)-3,6-dihydropyridine-1(2H)-carboxylate.

Step 4: Benzyl 4-(7-amino-3-(2,6-dioxopiperidin-3-yl)-2-oxo-2,3-dihydrobenzo[d]oxazol-6-yl)piperidine-1-carboxylate To a stirred solution of benzyl 4-(7-amino-3-(2,6-dioxopiperidin-3-yl)-2-oxo-2,3-dihydrobenzo[d]oxazol-6-yl)-3,6-dihydropyridine-1(2H)-carboxylate (1 eq) in ethyl acetate and THF is added platinum (IV) oxide (3 eq) and stirred under H2 (50 psi) in par shaker at room temperature until judged complete. A standard workup and purification provides benzyl 4-(7-amino-3-(2,6-dioxopiperidin-3-yl)-2-oxo-2,3-dihydrobenzo[d]oxazol-6-yl)piperidine-1-carboxylate.

Step 5: Benzyl 4-(7-(chlorosulfonyl)-3-(2,6-dioxopiperidin-3-yl)-2-oxo-2,3-dihydrobenzo[d]oxazol-6-yl)piperidine-1-carboxylate To a solution of benzyl 4-(7-amino-3-(2,6-dioxopiperidin-3-yl)-2-oxo-2,3-dihydrobenzo[d]oxazol-6-yl)piperidine-1-carboxylate (1 eq) in ethanol is added trifluoroborane;hydrofluoride (2 eq) and tert-butyl nitrite (2 eq) at 0° C. and stirred for 1 hour until judged complete. A standard workup and purification provides benzyl 4-(7-(chlorosulfonyl)-3-(2,6-dioxopiperidin-3-yl)-2-oxo-2,3-dihydrobenzo[d]oxazol-6-yl)piperidine-1-carboxylate.

Step 6: Benzyl 4-(3-(2,6-dioxopiperidin-3-yl)-7-(fluorosulfonyl)-2-oxo-2,3-dihydrobenzo[d]oxazol-6-yl)piperidine-1-carboxylate To a solution of benzyl 4-(7-(chlorosulfonyl)-3-(2,6-dioxopiperidin-3-yl)-2-oxo-2,3-dihydrobenzo[d]oxazol-6-yl)piperidine-1-carboxylate (1 eq) in anhydrous methanol is added sodium metabisulfite (2 eq) and purged with N2 for 5 minutes. Selectfluor (2 eq) is added and the reaction mixture is stirred at 70° C. for 12 hours. A standard workup and purification provide benzyl 4-(3-(2,6-dioxopiperidin-3-yl)-7-(fluorosulfonyl)-2-oxo-2,3-dihydrobenzo[d]oxazol-6-yl)piperidine-1-carboxylate.

Step 7: 3-(2,6-dioxopiperidin-3-yl)-2-oxo-6-(piperidin-4-yl)-2,3-dihydrobenzo[d]oxazole-7-sulfonyl fluoride (A-117) To a stirred solution of benzyl 4-(3-(2,6-dioxopiperidin-3-yl)-7-(fluorosulfonyl)-2-oxo-2,3-dihydrobenzo[d]oxazol-6-yl)piperidine-1-carboxylate (1 eq) in DCM is added TFA (1.2 eq) at 0° C. and stirred at room temperature until judged complete. A standard workup and purification provide 3-(2,6-dioxopiperidin-3-yl)-2-oxo-6-(piperidin-4-yl)-2,3-dihydrobenzo[d]oxazole-7-sulfonyl fluoride (A-117).

Synthesis A115: Synthesis of 3-(2,6-dioxopiperidin-3-yl)-2-oxo-6-(piperidin-4-yl)-2,3dihydrobenzo[d]oxazol-7-yl sulfurofluoridate (A-118):

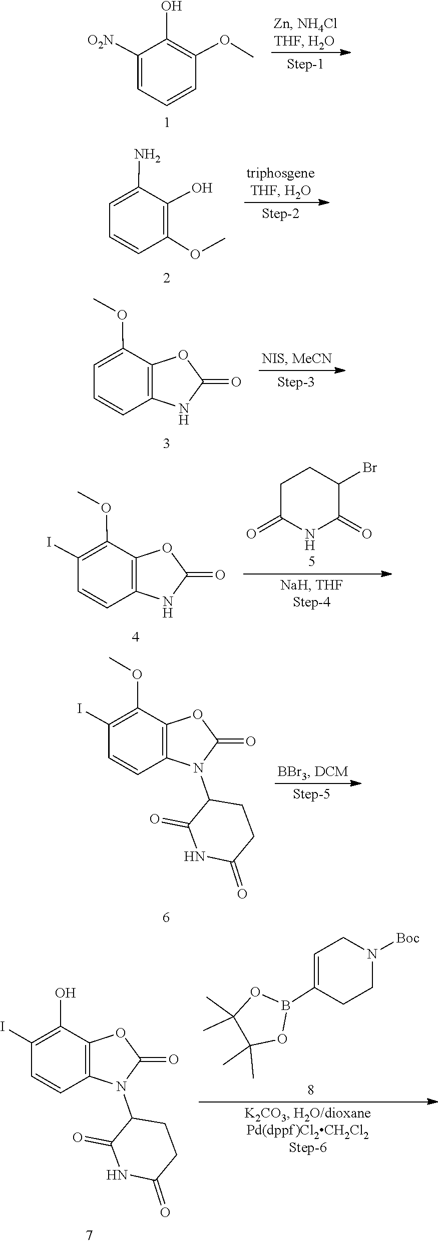

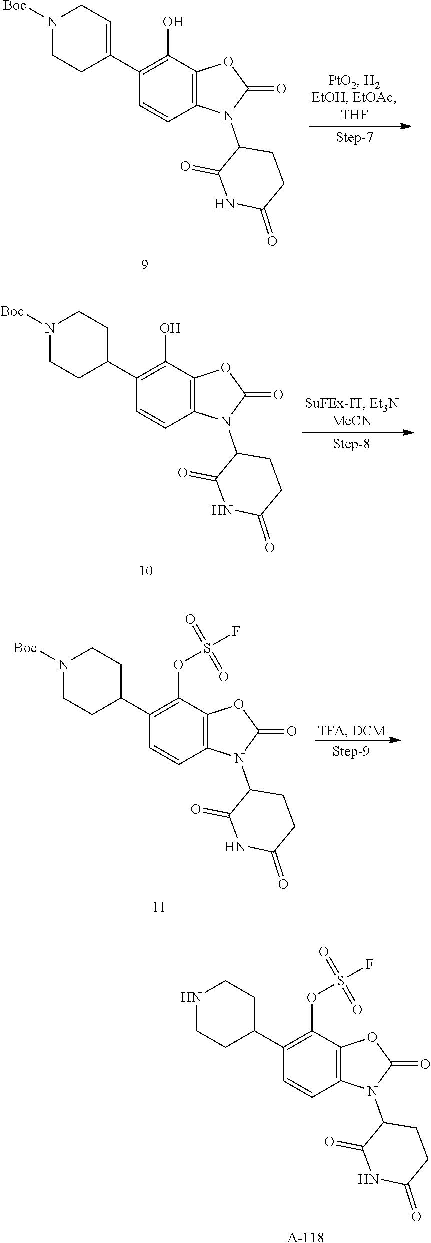 [SMILES: CC(C)(C)OC(=O)N1CC=C(c2ccc3c(oc(=O)n3C3CCC(=O)NC3=O)c2O)CC1 ; CC(C)(C)OC(=O)N1CCC(c2ccc3c(oc(=O)n3C3CCC(=O)NC3=O)c2O)CC1 ; CC(C)(C)OC(=O)N1CCC(c2ccc3c(oc(=O)n3C3CCC(=O)NC3=O)c2OS(=O)(=O)F)CC1 ; O=C1CCC(n2c(=O)oc3c(OS(=O)(=O)F)c(C4CCNCC4)ccc32)C(=O)N1 ; "A-118"]

To a stirred solution of 2-methoxy-6-nitro-phenol 1 (10 g, 59.12 mmol) in THF (150 mL) and water (50 mL) were added Zinc dust (19.22 g, 295.62 mmol) and ammonium chloride 5 (15.81 g, 295.62 mmol) at 0° C. and the reaction was stirred at room temperature for 1 h. After the completion of reaction, the reaction mixture was filtered and washed with ethyl acetate (500 mL×2). The combined organic layer dried over anhydrous sodium sulfate and concentrated under reduced pressure to give the crude product, which was purified by flash column chromatography (Davisil silica, 20% ethyl acetate in pet-ether as eluent) to afford 2-amino-6-methoxy-phenol 2 (8.5 g, 96% yield) as a brown solid. LC-MS (ES*): m z 139.91 [M+H]+Step-2: To a stirred solution of 2-amino-6-methoxy-phenol 2 (8.4 g, 60.37 mmol) in THF (150 mL) was added triphosgene (26.87 g, 90.55 mmol) at room temperature and the reaction was 15 stirred at 70° C. for 16 h. After completion of the reaction, the reaction mixture was cooled to room temperature, quenched with ice-cold water (500 mL), and extracted with ethyl acetate (500 mL×2). The combined organic layer was dried over sodium sulfate and concentrated under reduced pressure to give the crude product, which was purified by flash column chromatography (Davisil silica; 30% ethyl acetate in pet-ether as eluent) to afford 7-methoxy-3H-1,3-benzoxazol-2-one 3 (8 g, 68% yield) as a brown solid. LC-MS (ES+): m z 165.8 [M+H]+Step-3: To a stirred solution of 7-methoxy-3H-1,3-benzoxazol-2-one 3 (8 g, 48.44 mmol) in acetonitrile (30 mL) was added N-iodosuccinimide (7.63 g, 33.91 mmol) at -20° C. and the reaction was stirred for 48 h. After completion of the reaction, the reaction mixture was diluted with ice-cold water (500 mL), filtered and washed with aqueous sodium thiosulfate solution (200 mL). The residue was dried under reduced pressure to give the crude product, which was purified by flash column chromatography (Davisil silica; 20% ethyl acetate in pet-ether as eluent) to afford 6-iodo-7-methoxy-3H-1,3-benzoxazol-2-one 4 (4.5 g, 29% yield) as an off-white solid. LC-MS (ES-): m z 290[M - H]-Step-4: To a stirred solution of 6-iodo-7-methoxy-3H-1,3-benzoxazol-2-one 4 (3.5 g, 12.03 mmol) in THF (60 mL) was added sodium hydride (60% dispersion in mineral oil) (2.41 g, 60.13 mmol) at 0° C., the reaction mixture was stirred at 70° C. for 1 h. Then 3-bromopiperidine-2,6-dione 5 (11.55 g, 60.13 mmol) was added at 0° C. and stirred at 70° C. for 16 h. After completion, the reaction was quenched with ice-cold water (50 mL) and extracted ethyl acetate (100 mL×2). The combined organic layer was dried over sodium sulfate and concentrated under reduced pressure to give the crude compound, which was purified by flash column chromatography (Davisil silica: 60% ethyl acetate in pet-ether as eluent) to afford 3-(6-iodo-7-methoxy-2-oxo-1,3-benzoxazol-3-yl)piperidine-2,6-dione 6 (2 g, 33% yield) as an off-white solid. LC-MS (ES-): m z 401.05[M - H]-Step-5: To a stirred solution of 3-(6-iodo-7-methoxy-2-oxo-1,3-benzoxazol-3-yl)piperidine-2,6-dione 6 (2 g, 4.97 mmol) in DCM (30 mL) was added boron tribromide (4.7 mL, 49.73 mmol) dropwise at −78° C. The resulting mixture was stirred at room temperature for 24 h. After completion, the resulting mixture was quenched with ice-cold water (100 mL) and extracted with ethyl acetate (200 mL×2). The combined organic layer was dried over sodium sulfate and concentrated under reduced pressure to give the crude compound, which was purified by flash column chromatography (Davisil silica, 70% ethyl acetate in pet-ether as an eluent) to afford 3-(7-hydroxy-6-iodo-2-oxo-1,3-benzoxazol-3-yl)piperidine-2,6-dione 7 (0.9 g, 36% yield) as a yellow solid. LCMS (ES*): m z 388.9[M+H]+Step-6: In an oven dried 30 mL sealed tube, a mixture of 3-(7-hydroxy-6-iodo-2-oxo-1,3-benzoxazol-3-yl)piperidine-2,6-dione 7 (0.9 g, 2.32 mmol), tert-butyl 4-(4,4,5,5-tetramethyl-1,3,2-dioxaborolan-2-yl)-3,6-dihydro-2H-pyridine-1-carboxylate 8 (1.43 g, 4.64 mmol) and potassium carbonate (801 mg, 5.80 mmol) in water (2 mL) and 1,4-dioxane (8 mL) was degassed with nitrogen gas for 20 min, before Pd(dppf)Cl12CH2Cl2 (189 mg, 0.231 mmol) was added at room temperature and stirred at 90° C. for 4 h. After completion of the reaction, the reaction mixture was cooled to room temperature and filtered through a pad of celite. The filtrate was diluted with water (50 mL) then extracted with ethyl acetate (200 mL×2). The combined organic layer was dried over sodium sulfate and concentrated under reduced pressure to give the crude compound, which was purified by flash column chromatography (Davisil silica; 50% ethyl acetate in pet-ether as an eluent) to afford tert-butyl 4-[3-(2,6-dioxo-3-piperidyl)-7-hydroxy-2-oxo-1,3-benzoxazol-6-yl]-3,6-dihydro-2H-pyridine-1-carboxylate 9 (0.35 g, 26% yield) as a grey solid. LC-MS (ES-): m z 442.02[M - H]-Step-7: A solution of tert-butyl 4-[3-(2,6-dioxo-3-piperidyl)-7-hydroxy-2-oxo-1,3-benzoxazol-6-yl]-3,6-dihydro-2H-pyridine-1-carboxylate 9 (0.35 g, 0.789 mmol) in ethanol (7 mL), ethyl acetate (7 mL) and THF (7 mL) was degassed with nitrogen gas for 10 min with stirring. Then dioxoplatinum (179 mg, 0.789 mmol) was added at room temperature and the reaction mixture was stirred under hydrogen atmosphere in a parr shaker (60 psi) for 16 h. 10 After completion of the reaction, the reaction mixture was filtered through a pad of celite and washed with ethyl acetate (50 mL×2). The filtrate was concentrated under reduced pressure to afford tert-butyl 4-[3-(2,6-dioxo-3-piperidyl)-7-hydroxy-2-oxo-1,3-benzoxazol-6-yl]piperidine-1-carboxylate 10 (0.3 g, 64% yield) as an off-white solid. LC-MS (ES-): m z 444.1[M - H]-Step-8: To a stirred solution of tert-butyl 4-[3-(2,6-dioxo-3-piperidyl)-7-hydroxy-2-oxo-1,3-benzoxazol-6-yl]piperidine-1-carboxylate 10 (0.2 g, 0.448 mmol) in acetonitrile (4 mL) were added triethylamine (187 L, 1.35 mmol) and 1-(fluorosulfonyl)-2,3-dimethyl-1H-imidazol-3-ium trifluoromethanesulfonate (221 mg, 0.673 mmol) at room temperature and the reaction mixture was stirred for 2 h. After completion of the reaction, the reaction mixture was concentrated under reduced pressure to give the crude product, which was purified by Prep-SFC to afford tert-butyl 4-[3-(2,6-dioxo-3-piperidyl)-7-fluorosulfonyloxy-2-oxo-1,3-benzoxazol-6-yl]piperidine-1-carboxylate 11 (0.022 g, 9% yield) as a brown gum. LC-MS (ES-): m z 526.05 [M - H]-Preparative SFC Conditions: Column/dimensions: DCPAK P4VP (30x250) mm; 5p % CO2: 75%; % Co solvent: 25% (Acetonitrile: IPA) (1:1); Flow: 100 mL/min; Back Pressure: 100 bar; Temperature: 30° C.; UV: 220 nm; Solubility: Acetonitrile+THF.

Step-9: To a stirred solution of tert-butyl 4-[3-(2,6-dioxo-3-piperidyl)-7-fluorosulfonyloxy-2-oxo-1,3-benzoxazol-6-yl]piperidine-1-carboxylate 11 (0.02 g, 0.037 mmol) in DCM (2 mL) was added TFA (0.1 mL, 1.31 mmol) at 0° C., and the reaction mixture was stirred at room temperature for 2 h. After completion of the reaction, the reaction mixture was concentrated under reduced pressure to give the crude, which was triturated by diethyl ether (10 mL) and lyophilized to afford 3-(2,6-dioxo-3-piperidyl)-7-fluorosulfonyloxy-2-oxo-6-(4-piperidyl)-1,3-benzoxazole A-118 (14 mg, 66% yield, TFA salt) as a white solid. LC-MS (ES*): m z 428.29 [M+H]+. ¹H NMR (400 MHz, DMSO-d6): δ 11.28 (s, 1H), 8.57-8.57 (m, 1H), 7.48 (d, J=7.40 Hz, 1H), 7.27 (d, J=7.10 Hz, 1H), 3.74 (d, J=3.4, Hz, 1H), 3.63 (d, J=3.5 Hz, 1H), 5.48-5.43 (m, 1H), 3.47 (m, 2H), 3.26-3.20 (m, 1H), 3.15-3.08 (m, 2H), 2.94-2.85 (m, 1H), 2.69-2.61 (m, 2H), 1.93-1.86 (m, 4H) ppm. ¹⁹F NMR (376 MHz, DMSO-d6): 6 42.49 ppm.

Synthesis A116: Synthesis of 3-(2,6-dioxopiperidin-3-yl)-6-(piperidin-4-yl)benzo[d]isoxazol-7-yl sulfurofluoridate (A-119):

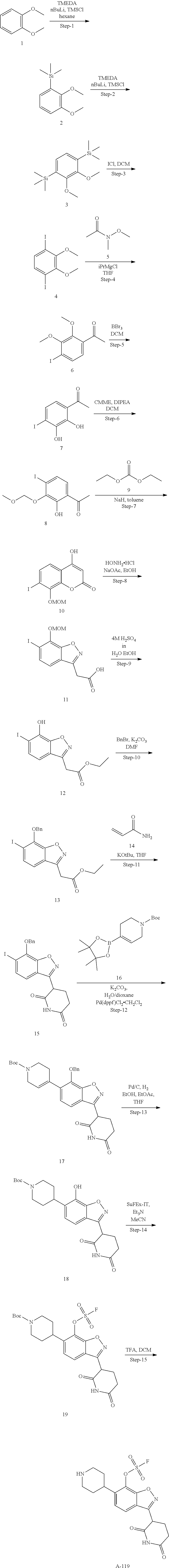

Step-1: To a stirred solution of 1,2-dimethoxybenzene 1 (10 g, 72.38 mmol) in hexane (100 mL) were added TMEDA (10.8 mL, 72.38 mmol) and 2.5 M n-BuLi in hexanes (33 mL, 79.62 5 mmol) at room temperature and stirred for 28 h, before TMSCl (11 mL, 86.85 mmol) was added dropwise at −78° C. and stirred at room temperature for 5 h. After completion of the reaction, the reaction mixture was quenched with ice-cold water (100 mL) and extracted with hexane (100 mL×2). The combined organic layer was dried over anhydrous sodium sulfate, filtered and concentrated under reduced pressure to give the crude product, which was purified 10 by flash column chromatography (Davisil silica, pet ether as an eluent) to afford (2,3-dimethoxyphenyl)-trimethyl-silane 2 (10 g, 62% yield) as a colorless oil. H-NMR (400 MHz, DMSO-d6): δ 7.07 (t, J=7.6 Hz, 1H), 6.98-6.94 (m, 2H), 3.86 (s, 3H), 3.85 (s, 3H), 0.28 (s, 9H) ppm.

Step-2: To a stirred solution of (2,3-dimethoxyphenyl)-trimethyl-silane 2 (10 g, 47.54 mmol) were added TMEDA (7 mL, 47.54 mmol) and 2.5 M n-BuLi in hexanes (21 mL, 52.29 mmol) at 0° C. and stirred at room temperature for 28 h, before TMSCl (7.2 mL, 57.05 mmol) was added dropwise at −78° C. and stirred at room temperature for 5 h. After completion of the reaction, the reaction mixture was quenched with ice cold water (200 mL) and extracted 20 with ethyl acetate (200 mL×2), the combined organic layer was dried over anhydrous sodium sulfate, filtered and concentrated under reduced pressure to give the crude, which was purified by flash column chromatography (Davisil silica, pet ether as an eluent) to afford (2,3-dimethoxy-4-trimethylsilyl-phenyl)-trimethyl-silane 3 (10 g, 71% yield) as a colorless oil. H-NMR (400 MHz, CDCl3) 6: 7.11 (s, 2H), 3.83 (s, 6H), 0.28 (s, 18H) ppm. 25 Step-3: To a stirred solution of (2,3-dimethoxy-4-trimethylsilyl-phenyl)-trimethyl-silane 3 (10 g, 35.40 mmol) in DCM (100 mL) was added iodine monochloride (11.5 g, 70.79 mmol) at 0° C. and stirred at room temperature for 1 h. After completion of reaction, the reaction mixture was quenched with sat. solution of sodium thiosulfate in water (200 mL) and extracted with DCM (200 mL×2). The combined organic layer was dried over anhydrous sodium sulfate and concentrated under reduced pressure to give the crude, which was purified by flash column chromatography (Davisil silica, 10% ethyl acetate in pet ether as an eluent) to afford 1,4-diiodo-2,3-dimethoxy-benzene 4 (8 g, 55% yield) as a colorless oil. H-NMR (400 MHz, CDCl3) 6: 7.24 (s, 2H), 3.87 (s, 6H) ppm.

Step-4: To a stirred solution of 1,4-diiodo-2,3-dimethoxy-benzene 4 (8 g, 20.52 mmol) in THF (80 mL) added 2 M isopropylmagnesium chloride in THF (10.2 mL, 20.52 mmol) at −50° C. and stirred for 1 h, before N-methoxy-N-methylacetamide 5 (2.54 g, 24.62 mmol) was added at −50° C. and stirred for 2 h. After completion of the reaction, the reaction mixture was warmed up to room temperature, quenched with ice cold water (100 mL) and extracted with ethyl acetate (100 mL×2). The combined organic layer was dried over anhydrous sodium sulfate, filtered and concentrated under reduced pressure to give the crude, which was purified by flash column chromatography (Davisil silica, 20% ethyl acetate in pet ether as an eluent) to afford 1-(4-iodo-2,3-dimethoxy-phenyl)ethanone 6 (3 g, 40% yield) as a colorless oil. LC-MS (ES*): m z 307.05 [M+H]+Step-5: To a stirred solution of 1-(4-iodo-2,3-dimethoxy-phenyl)ethanone 6 (2.3 g, 7.51 mmol) in DCM (30 mL) was added boron tribromide (4 mL, 15.03 mmol) at 0° C. and stirred at room temperature for 16 h. After completion of the reaction, the reaction mixture was diluted with ice cold water (30 mL) and extracted with DCM (20 mL×2). The combined organic layer was dried over anhydrous sodium sulfate and concentrated under reduced pressure to give the crude, which was purified by flash column chromatography (Davisil silica, 100% ethyl acetate as an eluent) to afford 1-(2,3-dihydroxy-4-iodo-phenyl)ethanone 7 (1 g, 47% yield) as an off-white solid. LC-MS (ES+): m z 278.85 [M+H]+Step-6: To a stirred solution of 1-(2,3-dihydroxy-4-iodo-phenyl)ethanone 7 (1 g, 3.60 mmol) in DCM (10 mL) were added DIPEA (1.2 mL, 7.19 mmol) and chloro(methoxy)methane (333 mg, 4.14 mmol) at −10° C. and stirred for 1 h. After completion of the reaction, the reaction mixture was diluted with water (20 mL) and extracted with DCM (20 mL×2). The combined organic layer was dried over anhydrous sodium sulfate and concentrated under reduced pressure to afford 1-[2-hydroxy-4-iodo-3-(methoxymethoxy)phenyl]ethanone 8 (1 g, 80% yield) as a colorless oil. LC-MS (ES-): m z 320.81 [M - H]-Step-7:]To a stirred solution of 1-[2-hydroxy-4-iodo-3-(methoxymethoxy)phenyl]ethanone 8 (1 g, 3.10 mmol) and diethyl carbonate 9 (1.9 mL, 15.52 mmol) in toluene (10 mL) was added sodium hydride (60% dispersion in oil) (373 mg, 15.52 mmol) at 0° C. and stirred at 100° C. for 16 h. After completion of reaction, the reaction mixture was diluted with ice-cold water (20 mL) and extracted with ethyl acetate (20 mL×2). The combined organic layer was dried over anhydrous sodium sulfate and concentrated under reduced pressure to give the crude, which was purified by flash column chromatography (Davisil silica, ethyl acetate as an eluent) to afford 4-hydroxy-7-iodo-8-(methoxymethoxy)chromen-2-one 10 (0.5 g, 44% yield) as a colorless oil. LC-MS (ES-): m z 347.10 [M - H]-Step-8: To stirred a solution of 4-hydroxy-7-iodo-8-(methoxymethoxy)chromen-2-one 10 (0.5 g, 1.44 mmol) in ethanol (5 mL) were added sodium acetate (354 mg, 4.31 mmol) and hydroxylamine hydrochloride (299 mg, 4.31 mmol) at room temperature and stirred at 90° C. for 4 h. After completion of the reaction, the reaction mixture was diluted with water (10 mL) and extracted with ethyl acetate (10×2 mL). The combined organic layer was washed with brine (10 mL), dried over anhydrous sodium sulfate, filtered and concentrated under reduced pressure to afford 2-[6-iodo-7-(methoxymethoxy)-1,2-benzoxazol-3-yl]acetic acid 11 (0.5 g, 75% yield) as a brown solid. LC-MS (ES+): m z 363.91 [M+H]+Step-9: To stirred a solution of 2-[6-iodo-7-(methoxymethoxy)-1,2-benzoxazol-3-yl]acetic acid 11 (0.5 g, 1.38 mmol) in ethanol (5 mL) added 4 M solution of sulfuric acid in water (1.50 mL, 6 mmol) at 0° C. and stirred at 90° C. for 4 h. After completion of the reaction, the reaction mixture was basified with saturated sodium bicarbonate solution (10 mL) and extracted with ethyl acetate (10 mL×2). The combined organic layer was washed with brine (10 mL), dried over anhydrous sodium sulfate and concentrated under reduced pressure to afford ethyl 2-(7-hydroxy-6-iodo-1,2-benzoxazol-3-yl)acetate 12 (0.35 g, 57% yield) as a brown liquid. LC-MS (ES+): m z 348.08 [M+H]+Step-10: To a stirred solution of ethyl 2-(7-hydroxy-6-iodo-1,2-benzoxazol-3-yl)acetate 12 (0.35 g, 1.01 mmol) in DMF (5 mL) were added potassium carbonate (418 mg, 3.03 mmol) and benzyl bromide (0.25 mL, 2.02 mmol) at 0° C. and stirred at room temperature for 1 h. After completion of the reaction, the reaction mixture was diluted with water (5 mL) and extracted with ethyl acetate (5 mL×2). The combined organic layer was dried over anhydrous sodium sulfate and concentrated under reduced pressure to give the crude, which was purified by flash column chromatography (Davisil silica, 10% ethyl acetate in pet ether 5 as an eluent) to afford ethyl 2-(7-benzyloxy-6-iodo-1,2-benzoxazol-3-yl)acetate 13 (0.36 g, 69% yield) as an off-white solid, LC-MS (ES*): m z 438.59 [M+H]+Step-11: To a stirred solution of ethyl 2-(7-benzyloxy-6-iodo-1,2-benzoxazol-3-yl)acetate 13 (0.36 g, 0.823 mmol) and prop-2-enamide 14 (70 mg, 0.988 mmol) in THF (5 mL) was 10 added potassium tert-butoxide (92 mg, 0.823 mmol) at 0° C. and the reaction mixture was stirred at room temperature for 30 minutes. After completion of the reaction, the reaction mixture was quenched with sat. solution of ammonium chloride in water (5 mL) and extracted with ethyl acetate (5 mL×2). The combined organic layer was dried over anhydrous sodium sulfate, filtered and concentrated under reduced pressure to give the crude, which was purified by flash column chromatography (Davisil silica, 40% ethyl acetate in pet ether as an eluent) to afford 3-(7-benzyloxy-6-iodo-1,2-benzoxazol-3-yl)piperidine-2,6-dione 15 (0.22 g, 49% yield) as an off-white solid. LC-MS (ES-): m z 460.88 [M - H]-Step-12: To a stirred solution of 3-(7-benzyloxy-6-iodo-1,2-benzoxazol-3-yl)piperidine-2,6-dione 15 (220 mg, 0.475 mmol) and tert-butyl 4-(4,4,5,5-tetramethyl-1,3,2-dioxaborolan-2-yl)-3,6-dihydro-2H-pyridine-1-carboxylate 16 (177 mg, 0.571 mmol) in 1,4 dioxane (3.5 mL) and water (0.5 mL) added potassium carbonate (164 mg, 1.19 mmol) and degassed with Nitrogen gas for 5 mins before addition of Pd(dppf)Cl12CH2Cl2 (39 mg, 0.047 mmol) and stirred at 90° C. for 4 h. After completion of the reaction, the reaction mixture was diluted with water (4 mL) and extracted with ethyl acetate (4 mL×2). The combined organic layer was washed with brine (5 mL), dried over anhydrous sodium sulfate and concentrated under reduced pressure. The crude was purified by column chromatography (Davisil silica, ~60% ethyl acetate in pet ether as an eluent) to afford tert-butyl 4-[7-benzyloxy-3-(2,6-dioxo-3-piperidyl)-1,2-benzoxazol-6-yl]-3,6-dihydro-2H-pyridine-1-carboxylate 17 (120 mg, 38% yield) as an off-white solid. LC-MS (ES-): m z 516.14 [M - H]-Step-13: To a stirred solution of tert-butyl 4-[7-benzyloxy-3-(2,6-dioxo-3-piperidyl)-1,2-benzoxazol-6-yl]-3,6-dihydro-2H-pyridine-1-carboxylate 17 (80 mg, 0.154 mmol) in ethanol (2 mL), ethyl acetate (2 mL) and THF (2 mL) was added 10% palladium on carbon (82 mg, 0.772 mmol) and the reaction mixture was stirred at room temperature under hydrogen atmosphere ~15 psi (hydrogen balloon) for 3 hours. After completion of the reaction, the reaction mixture was filtered through a pad of celite and concentrated under reduced pressure to afford tert-butyl 4-[3-(2,6-dioxo-3-piperidyl)-7-hydroxy-1,2-benzoxazol-6-yl]piperidine-1-carboxylate 18 (60 mg, 42% yield) as an off-white solid. LC-MS (ES-): m z 430.15 [M+H]+Step-14: To a stirred solution of tert-butyl 4-[3-(2,6-dioxo-3-piperidyl)-7-hydroxy-1,2-benzoxazol-6-yl]piperidine-1-carboxylate 18 (60 mg, 0.139 mmol) in acetonitrile (2 mL) were added Et3N (0.1 mL, 0.419 mmol), 1-(fluorosulfonyl)-2,3-dimethyl-1H-imidazol-3-ium trifluoromethanesulfonate (69 g, 0.209 mmol) at 0° C. and the reaction mixture was stirred at room temperature for 1 hour. After completion of the reaction, the reaction mixture was diluted with water (1 mL) and extracted with ethyl acetate (2 mL×2). The combined organic layer was washed with brine solution (2 mL), dried over anhydrous sodium sulfate and concentrated under reduced pressure to give the crude product, which was purified by achiral SFC to afford tert-butyl 4-[3-(2,6-dioxo-3-piperidyl)-7-fluorosulfonyloxy-1,2-benzoxazol-6-yl]piperidine-1-carboxylate 19 (6 mg, 8% yield) as an off-white solid. LC-MS (ES-): m z 510.40 [M - H]-SFC Method: Column/dimensions: Ethyl Pyridine (4.6x250)mm,5p; % CO2: 75%; % CO solvent: 25% (Acetonitrile:IPA)(1:1); Flow: 3 mL/min; Back Pressure: 100 bar; Temperature: 30° C.; UV: 220 nm.

Step-15: To a stirred solution of tert-butyl 4-[3-(2,6-dioxo-3-piperidyl)-7-fluorosulfonyloxy-1,2-benzoxazol-6-yl]piperidine-1-carboxylate 19 (6 mg, 0.011 mmol) in DCM (2 mL) was added TFA (0.05 mL, 0.653 mmol) at 0° C. and stirred at room temperature for 1 hour. After completion of the reaction, the reaction mixture was concentrated under reduced pressure to give the crude, which was triturated with diethyl ether (2 mL×2) and dried under reduced pressure to afford 3-(2,6-dioxo-3-piperidyl)-7-fluorosulfonyloxy-6-(4-piperidyl)-1,2-benzoxazole A-119 (6 mg, 90% yield, TFA salt) as a white solid, LC-MS (ES+): m z 412.31 [M+H]+. ¹H NMR (400 MHz, DMSO-d6): δ 11.18 (s, 1H), 8.41 (br s, 2H), 8.07 (d, J=8.4 Hz, 1H), 7.47 (d, J=8.4 Hz, 1H), 4.70 (dd, J=4.8 Hz, 1H), 3.43-3.32 (m, 3H), 3.18-3.12 (m, 2H), 2.83-2.76 (m, 1H), 2.67-2.59 (m, 2H), 2.50-2.21 (m, 1H), 1.97-1.92 (m, 4H) ppm. ¹⁹F NMR (376 MHz, DMSO-d6): δ 42.373 ppm.

Synthesis A117: Synthesis of 3-(2,6-dioxopiperidin-3-yl)-6-(piperidin-4-yl)benzo[d]isoxazole-7-sulfonyl fluoride (A-120):

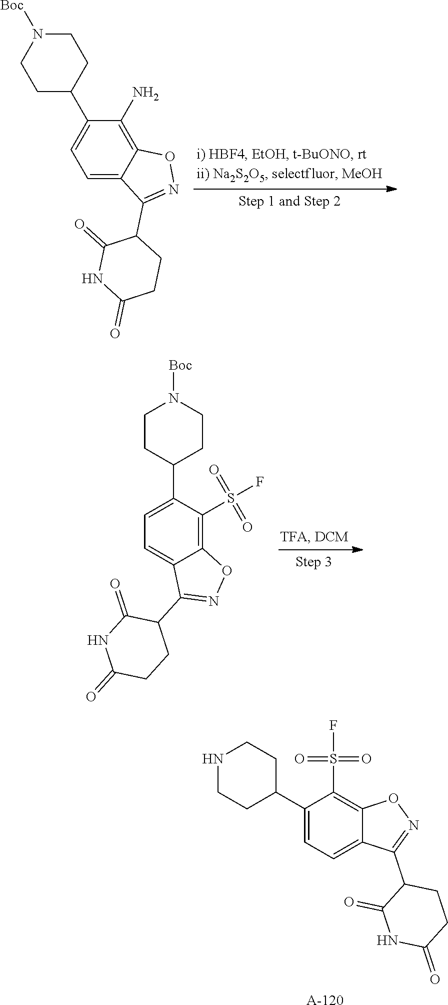 [SMILES: CC(C)(C)OC(=O)N1CCC(c2ccc3c(C4CCC(=O)NC4=O)noc3c2N)CC1 ; CC(C)(C)OC(=O)N1CCC(c2ccc3c(C4CCC(=O)NC4=O)noc3c2S(=O)(=O)F)CC1 ; O=C1CCC(c2noc3c(S(=O)(=O)F)c(C4CCNCC4)ccc23)C(=O)N1 ; "A-120"]

Step 1 and Step 2: tert-butyl 4-(3-(2,6-dioxopiperidin-3-yl)-7- ^{P}GP-⁷⁴⁸,C³ (fluorosulfonyl)benzo[d]isoxazol-6-yl)piperidine-1-carboxylate To a solution of tert-butyl 4-(7-amino-3-(2,6-dioxopiperidin-3-yl)benzo[d]isoxazol-6-yl)piperidine-1-carboxylate (1 eq) in ethanol is added trifluoroborane;hydrofluoride (2 eq) and tert-butyl nitrite (2 eq) at 0° C. and stirred for 1 hour until judged complete. A standard workup and purification provides tert-butyl 4-(3-(2,6-dioxopiperidin-3-yl)-7-((tetrafluoro-15-boraneyl)diazenyl)benzo[d]isoxazol-6-yl)piperidine-1-carboxylate. To a solution of 2-(2,6-dioxopiperidin-3-yl)-3-oxo-2,3-dihydro-1H-pyrrolo[3,4-c]pyridine-7-sulfonyl chloride (1 eq) in anhydrous methanol is added sodium metabisulfite (2 eq) and purged with N2 for 5 minutes.

Selectfluor (2 eq) is added and the reaction mixture is stirred at 70° C. for 12 hours. A standard workup and purification provide tert-butyl 4-(3-(2,6-dioxopiperidin-3-yl)-7-(fluorosulfonyl)benzo[d]isoxazol-6-yl)piperidine-1-carboxylate.

Step 3: 3-(2,6-dioxopiperidin-3-yl)-6-(piperidin-4-yl)benzo[d]isoxazole-7-sulfonyl fluoride (A-120) To a stirred solution of tert-butyl 4-(3-(2,6-dioxopiperidin-3-yl)-7-(fluorosulfonyl)benzo[d]isoxazol-6-yl)piperidine-1-carboxylate (1 eq) in DCM is added TFA (1.2 eq) at 0° C. and stirred at room temperature until judged complete. A standard workup and purification provides 3-(2,6-dioxopiperidin-3-yl)-6-(piperidin-4-yl)benzo[d]isoxazole-7-sulfonyl fluoride (A-120).

Synthesis A118: Synthesis of 2-(4-(4-(2,6-dioxopiperidin-3-yl)-2-((fluorosulfonyl)oxy)phenyl)piperidin-1-yl)acetic acid (A-121)

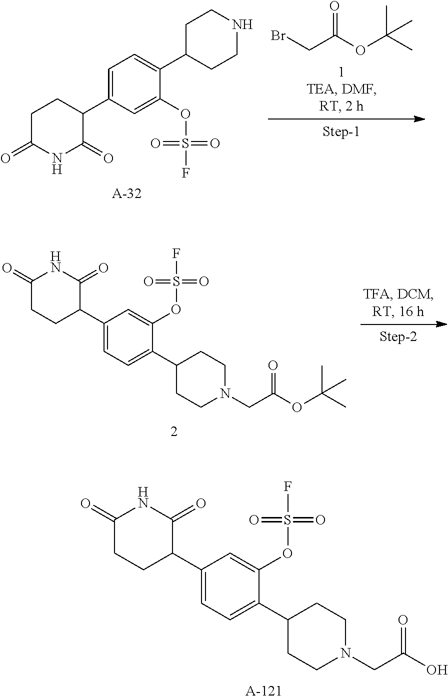

Step-1: tert-butyl 2-[4-[4-(2,6-dioxo-3-piperidyl)-2-fluorosulfonyloxy-phenyl]-1-piperidyl]acetate To a stirred solution of 3-[3-fluorosulfonyloxy-4-(4-piperidyl)phenyl]-2,6-dioxo-piperidine A-32 (0.5 g, 1.35 mmol) in dimethylformamide (8.40 mL) were added triethylamine (409.79 mg, 4.05 mmol, 564.45 L), followed by tert-butyl 2-bromoacetate 1(315.97 mg, 1.62 mmol, 237.57 L) at 0° C. and stirred at room temperature for 2 hours. After completion, the reaction mixture was diluted with ice cold water (20 mL), the precipitated solid was collected and dried under reduced pressure to afford tert-butyl 2-[4-[4-(2,6-dioxo-3-piperidyl)-2-fluorosulfonyloxy-phenyl]-1-piperidyl]acetate 2 (0.62 g, 73% yield) as an off-white solid. (The crude product was taken to next reaction without further purification.) LCMS (ES-): m z 483.36 15 [M-H]-.

Step-2: 2-[4-[4-(2,6-dioxo-3-piperidyl)-2-fluorosulfonyloxy-phenyl]-1-piperidyl]acetic acid (A-121) To a stirred solution of tert-butyl 2-[4-[4-(2,6-dioxo-3-piperidyl)-2-fluorosulfonyloxy-phenyl]-1-piperidyl]acetate 2 (0.6 g, 1.24 mmol) in DCM (6 mL) added TFA (1.69 g, 14.86 20 mmol, 1.14 mL) at 0° C. and stirred at room temperature for 16 hours. After completion, the reaction mixture was concentrated under reduced pressure to get crude product, which was purified by prep HPLC to afford 2-[4-[4-(2,6-dioxo-3-piperidyl)-2-fluorosulfonyloxy-phenyl]-1-piperidyl]acetic acid (A-121) (0.52 g, 76% yield, trifluoroacetic acid) as an off-white solid.

LCMS (ES*): 429.21 [M+H]+. Prep HPLC Method: Column: X-SELECT C18, 5 pm (150mmX19 mm) Mobile Phase A: 0.1% FA in water, B: 100% acetonitrile, Gradient (T % B): 0/5,2/5,12/55, Flow Rate: 18 ml/min, Sample Diluent: acetonitrile+water+tetrahydrofuran. H-NMR (400 MHz, DMSO-d6): δ 10.99 (s, 1H), 7.53 (d, J=5.2 Hz, 1H), 7.47 (s, 1H), 7.42 (d, J=8.4 Hz, 1H), 4.01 (q, J=5.6 Hz, 1H), 3.57 (br s, 2H), 3.34 (d, J=9.2 Hz, 2H), 2.99 (t, J=12 Hz, 1H), 2.84 (br s, 2H), 3.32 (m, 2H), 2.73-2.67 (m, 1H), 2.28-2.22 (m, 1H), 2.07 (s, 2H), 2.04-2.02 (m, 2H), 1.96-1.93 (m, 2H).

Synthesis Al19: Synthesis of 2-(4-(4-(2,6-dioxopiperidin-3-yl)-2-((fluorosulfonyl oxy phenyl)piperazin-1-yl)acetic acid (A-122)

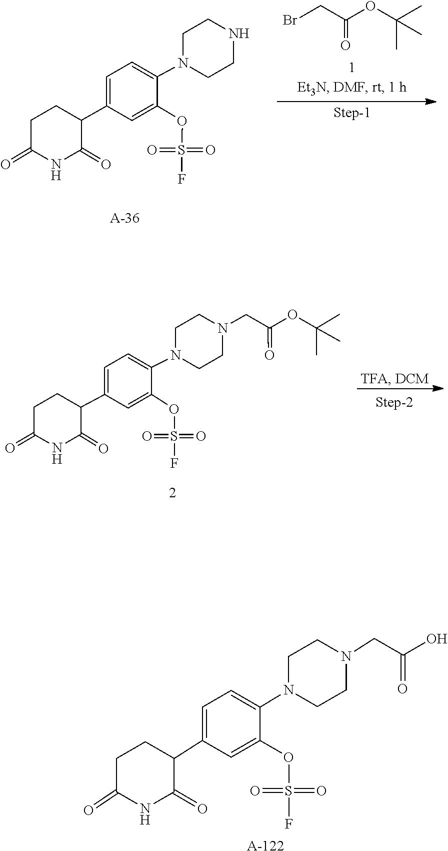

Step-1: tert-butyl 2-(4-(4-(2,6-dioxopiperidin-3-yl)-2-((fluorosulfonyl)oxy)phenyl)piperazin-1-yl)acetate To a stirred solution of 5-(2,6-dioxopiperidin-3-yl)-2-(piperazin-1-yl)phenyl 15 sulfurofluoridate A-36 (0.6 g, 1.24 mmol, Trifluoroacetic acid) in dimethylformamide (6 mL) added triethylamine (490.44 mg, 4.85 mmol, 675.54 L), tert-butyl 2-bromoacetate 3 (289.32 mg, 1.48 mmol, 217.54 L) consecutively and stirred for 1 hour at room temperature. After completion, the reaction mixture was quenched with ice cold water (10 mL). The solid obtained was filtered and washed with water to obtain the crude product of tert-butyl 2-[4-[4-(2,6-dioxo-3-piperidyl)-2-fluorosulfonyloxy-phenyl]piperazin-1-yl]acetate 2 (0.510 g, 78% yield) as an off-white solid. LCMS (ES*): m z 486.37 [M+H]+.

Step-2: 2-(4-(4-(2,6-dioxopiperidin-3-yl)-2-((fluorosulfonyl)oxy)phenyl)piperazin-1-yl)acetic acid (A-122) To a stirred solution of tert-butyl 2-[4-[4-(2,6-dioxo-3-piperidyl)-2-fluorosulfonyloxy-phenyl]piperazin-1-yl]acetate 2 (200 mg, 411.93 mol) in dichloromethane (2 mL) added trifluoroacetic acid (469.69 mg, 4.12 mmol, 0.2 mL) at 0° C. and stirred at room temperature for 16 hours. After completion, the reaction mixture was evaporated under reduced pressure and diluted with water (25 mL) and extracted with ethyl acetate (3×25 mL). The combined organic layer evaporated to obtain crude product of 2-[4-[4-(2,6-dioxo-3-piperidyl)-2-fluorosulfonyloxy-phenyl]piperazin-1-yl]acetic acid (A-122) (0.120 g, 62% yield) as an off-white solid. The crude compound has been forwarded to the next step without further purification. LCMS (ES*): m z 430.41 [M+H]+.

Synthesis A120: Synthesis of 2-(4-(4-(2,4-dioxotetrahydropyrimidin-1(2H)-yl)-2-((fluorosulfonyl)oxy)phenyl)piperidin-1-yl)acetic acid (A-123)

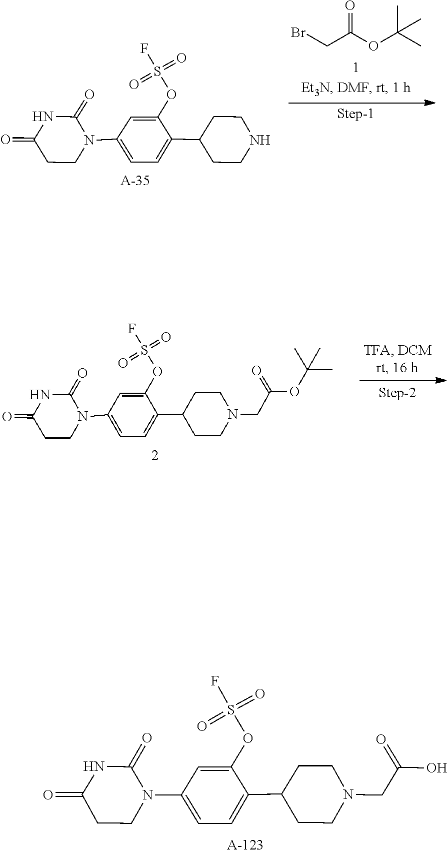

Step-1: tert-butyl 2-(4-(4-(2,4-dioxotetrahydropyrimidin-1(2H)-yl)-2-((fluorosulfonyl)oxy)phenyl)piperidin-1-yl)acetate To a stirred solution 5-(2,4-dioxotetrahydropyrimidin-1(2H)-yl)-2-(piperidin-4-yl)phenyl sulfurofluoridate A-35 (300 mg, 807.79 mol) in dimethylformamide (5 mL) added triethylamine (245.22 mg, 2.42 mmol, 337.77 pL), tert-butyl 2-bromoacetate 1 (189.08 mg, 969.35 mol, 142.17 pL) consecutively and stirred at room temperature for 1 hour. After completion, the reaction mixture was quenched with ice cold water (10 mL), the precipitate obtained was filtered and washed with water to obtain tert-butyl 2-[4-[4-(2,4-dioxohexahydropyrimidin-1-yl)-2-fluorosulfonyloxy-phenyl]-1-piperidyl]acetate 2 (260 mg, 65% yield) as an off-white solid. LCMS (ES*): m z 486.41 [M+H]+.

Step-2: 2-(4-(4-(2,4-dioxotetrahydropyrimidin-1(2H)-yl)-2-((fluorosulfonyl)oxy)phenyl)piperidin-1-yl)acetic acid (A-123) To a stirred solution of tert-butyl 2-[4-[4-(2,4-dioxohexahydropyrimidin-1-yl)-2-fluorosulfonyloxy-phenyl]-1-piperidyl]acetate 4 (260 mg, 535.50 mol) in dichloromethane (2 mL) was added trifluoroacetic acid (183.17 mg, 1.61 mmol, 122.94 L) and stirred at room temperature for 16 hours. After completion, the reaction mixture was evaporated under reduced pressure to obtain crude product. The crude product was triturated with diethyl ether (20 ml) to afford 2-[4-[4-(2,4-dioxohexahydropyrimidin-1-yl)-2-fluorosulfonyloxy-phenyl]-1-piperidyl]acetic acid A-123 (231 mg, 61% yield) as an off-white solid. LCMS (ES*): m z 430.32 [M+H]+.

Synthesis A121: Synthesis of 2-(4-(4-(2,4-dioxotetrahydropyrimidin-1(2H)-yl)-2-((fluorosulfonyl)oxy)phenyl)piperazin-1-yl)acetic

0 N O O=S=0 Step-1: O==0

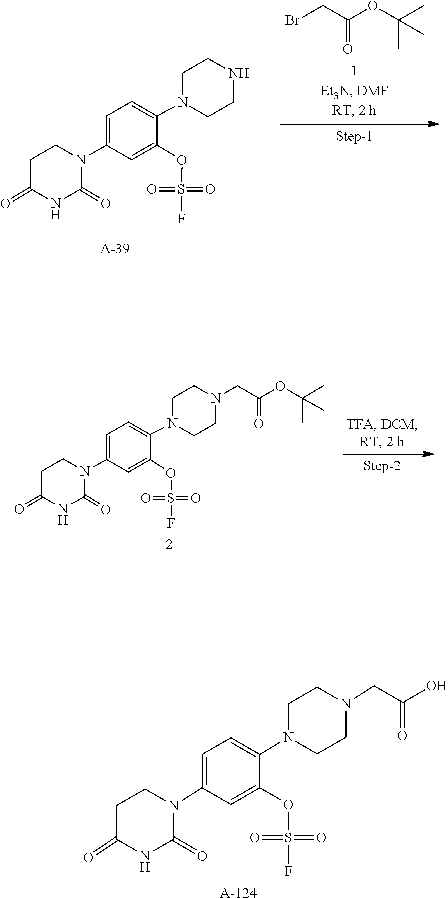

Step-1: tert-butyl 2-(4-(4-(2,4-dioxotetrahydropyrimidin-1(2H)-yl)-2-((fluorosulfonyl)oxy)phenyl)piperazin-1-yl)acetate To the stirred solution of 1-(3-fluorosulfonyloxy-4-piperazin-1-yl-phenyl)-2,4-dioxo-hexahydropyrimidine 1 (0.6 g, 1.61 mmol, trifluoroacetic acid) in dimethylformamide (6 mL) was added triethyl amine (326.09 mg, 3.22 mmol, 449.16 pL) and tert-butyl 2-bromoacetate (377.15 mg, 1.93 mmol, 283.57 L) at room temperature. The reaction mixture was stirred at room temperature for 2 hours. The progress of the reaction was monitored by LCMS. After completion of reaction, the reaction mixture was diluted with water (10 mL) and extracted with ethyl acetate (2×20 mL). The combined organic layer was washed with saturated sodium chloride solution (10 mL), dried over anhydrous sodium sulfate and concentrated under reduced pressure at 45° C. to get desired product of tert-butyl- 2-[4-[4-(2,4-dioxohexahydropyrimidin-1-yl)-2-fluorosulfonyloxy-phenyl]piperazin-1-yl]acetate 2 (0.45 g, 46% yield) as an off-white solid. LC-MS (ES*): m z 487.59 [M+H]+.

Step-2: 2-(4-(4-(2,4-dioxotetrahydropyrimidin-1(2H)-yl)-2-((fluorosulfonyl)oxy)phenyl)piperazin-1-yl)acetic acid (A-124)

To a stirred solution of tert-butyl 2-[4-[4-(2,4-dioxohexahydropyrimidin-1-yl)-2-fluorosulfonyloxy-phenyl]piperazin-1-yl]acetate 2 (0.45 g, 924.95 mol) in dichloromethane (5.00 mL) added trifluoroacetic acid (527.33 mg, 4.62 mmol, 353.91 L) and stirred at room temperature for 2 hours. After completion, the reaction mixture was concentrated under reduced pressure to get crude product, which was triturated with diethyl ether (10 mL) to afford 2-[4-[4-(2,4-dioxohexahydropyrimidin-1-yl)-2-fluorosulfonyloxy-phenyl]piperazin-1-yl]acetic acid A-124 (321 mg, 79% yield, trifluoroacetic acid) as an off-white solid. LC-MS (ES*): m z 431.33 [M+H]+.H-NMR (400 MHz, DMSO-d6): δ 10.47 (s, 1H), 7.62 (s, 1H), 7.45 (d, J=2.00 Hz, 1H), 7.38 (d, J=8.80 Hz, 1H), 4.19 (d, J=12.80 Hz, 2H), 3.80 (t, J=6.40 Hz, 2H), 3.31-3.45 (m, 4H), 3.09-3.10 (m, 2H), 2.70-2.72 (m, 2H), 1.18 (t, J=7.60 Hz, 3H).

Synthesis A122: Synthesis of 2-(4-(4-((2,6-dioxopiperidin-3-yl)oxy)-2-((fluorosulfonyl)oxy)phenyl)piperidin-1-yl)acetic acid (A-125)

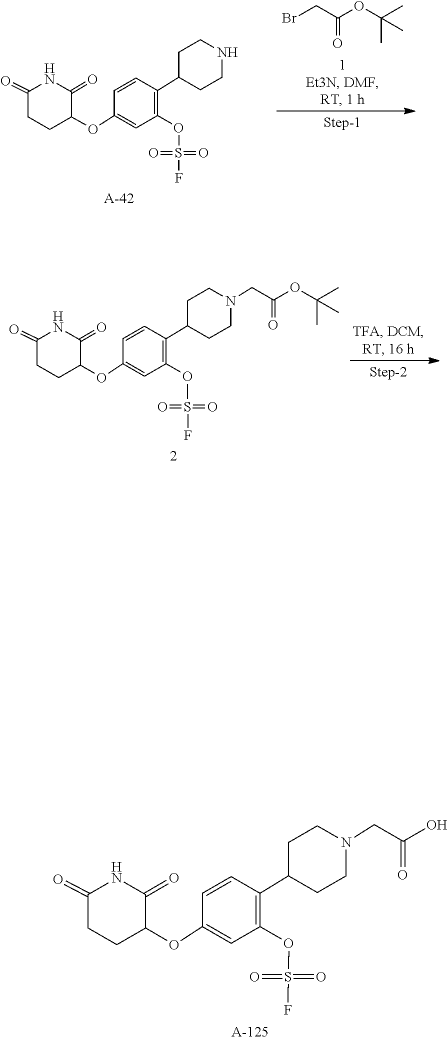

((fluorosulfonyl)oxy)phenyl)piperidin-1-yl)acetate To a stirred solution of 5-((2,6-dioxopiperidin-3-yl)oxy)-2-(piperidin-4-yl)phenyl sulfurofluoridate A-42 (0.6 g, 1.55 mmol) in dimethylformamide (3 mL) was added triethylamine (471.38 mg, 4.66 mmol, 649.29 L), followed by tert-butyl 2-bromoacetate 1 (363.46 mg, 1.86 mmol, 273.28 L) at 0° C. and stirred at room temperature for 1 hour. After completion of the reaction, the reaction mixture was diluted with chilled water (10 mL), the precipitated solid was filtered and dried under reduced pressure to afford tert-butyl 2-[4-[4-[(2,6-dioxo-3-piperidyl)oxy]-2-fluorosulfonyloxy-phenyl]-1-piperidyl]acetate 2 (0.6 g, 69% yield) as an off-white solid. (Crude product was taken to next reaction without further purification). LCMS (ES*): m z 501.60 [M+H]+.

Step-2: Synthesis of 2-(4-(4-((2,6-dioxopiperidin-3-yl)oxy)-2-((fluorosulfonyl)oxy)phenyl)piperidin-1-yl)acetic acid (A-125) To a stirred solution of tert-butyl 2-[4-[4-[(2,6-dioxo-3-piperidyl)oxy]-2-fluorosulfonyloxy-phenyl]-1-piperidyl]acetate 2 (0.2 g, 399.57 mol) in dichloromethane (3 mL) was added trifluoroacetic acid (227.80 mg, 2.00 mmol, 152.89 L) at 0° C. and stirred at room temperature for 16 hours. After completion of the reaction, the reaction mixture was 10 concentrated under reduced pressure to obtain crude product, which was purified by Prep HPLC purification to afforded 2-[4-[4-[(2,6-dioxo-3-piperidyl)oxy]-2-fluorosulfonyloxy-phenyl]-1-piperidyl]acetic acid A-125 (0.120 g, 264.61 mol, 66% yield) as an off-white solid.

LCMS (ES+): m z 445.33 [M+H]+.

Synthesis A123: Synthesis of 2-(4-(4-((2,6-dioxopiperidin-3-yl)oxy)-2-(fluorosulfonyl)phenyl)piperidin-1-yl)acetic acid (A-126)

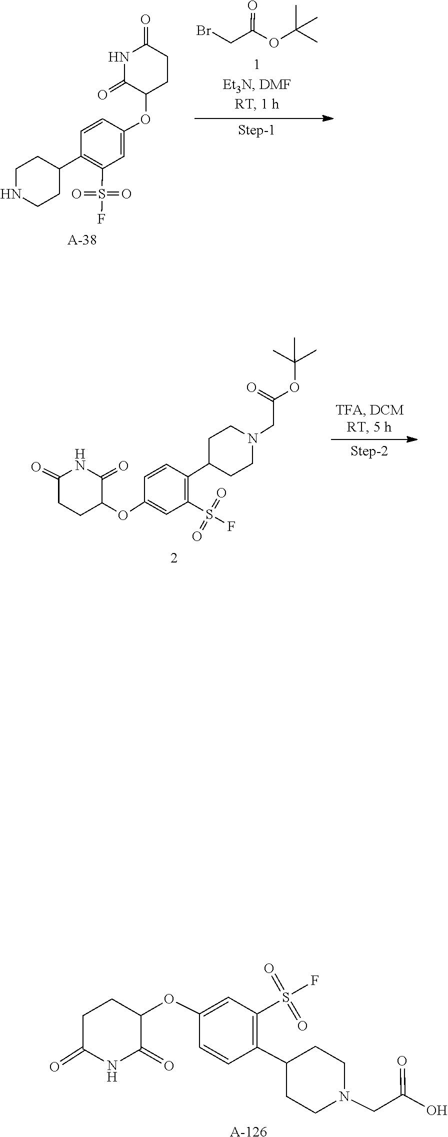

Step-1: tert-butyl 2-(4-(4-((2,6-dioxopiperidin-3-yl)oxy)-2-(fluorosulfonyl)phenyl)piperidin-1-yl)acetate To a stirred solution of 5-[(2,6-dioxo-3-piperidyl)oxy]-2-(4-piperidyl)benzenesulfonyl fluoride A-38 (0.2 g, 0.539 mmol) in dimethylformamide (4.0 mL) was added triethyl amine (225.78 pL, 1.62 mmol) at room temperature and stirred for 5 minutes. Then added tert-butyl 2-bromoacetate 1 (95 L, 0.647 mmol) into the reaction mixture at 0° C. and stirred at room temperature for 1 hour. After completion of the reaction, the reaction mixture was diluted with ice-cold water (10 mL), the solid obtained was filtered and washed with water and dried under reduced pressure to afford tert-butyl 2-[4-[4-[(2,6-dioxo-3-piperidyl)oxy]-2-fluorosulfonyl-phenyl]-1-piperidyl]acetate 2 (0.15 g, 41% yield) as an off-white solid. LCMS (ES*): m z 5 485.49 [M+H]+.

Step-2: 2-(4-(4-((2,6-dioxopiperidin-3-yl)oxy)-2-(fluorosulfonyl)phenyl)piperidin-1-yl)acetic acid (A-126) To a stirred solution of tert-butyl 2-[4-[4-[(2,6-dioxo-3-piperidyl)oxy]-2-fluorosulfonyl-phenyl]-1-piperidyl]acetate 2 (0.14 g, 0.288 mmol) in DCM (2 mL) was added trifluoroacetic acid (331 L, 4.33 mmol) at 0° C. and stirred at room temperature for 5 hours.

After completion of the reaction, the reaction mixture was concentrated under reduced pressure to obtain crude product, which was triturated with pentane (2×10 mL) and dried under reduced pressure to afford 2-[4-[4-[(2,6-dioxo-3-piperidyl)oxy]-2-fluorosulfonyl-phenyl]-1-piperidyl]acetic acid A-126 (0.12 g, 66% yield, trifluoroacetic acid) as an off-white solid. 15 LCMS (ES+): m z 429.13 [M+H]+.

Synthesis A124: Synthesis of 2-(4-(4-((2,6-dioxopiperidin-3-yl)amino)-2-(fluorosulfonyl)phenyl)piperidin-1-yl)acetic acid (A-127)

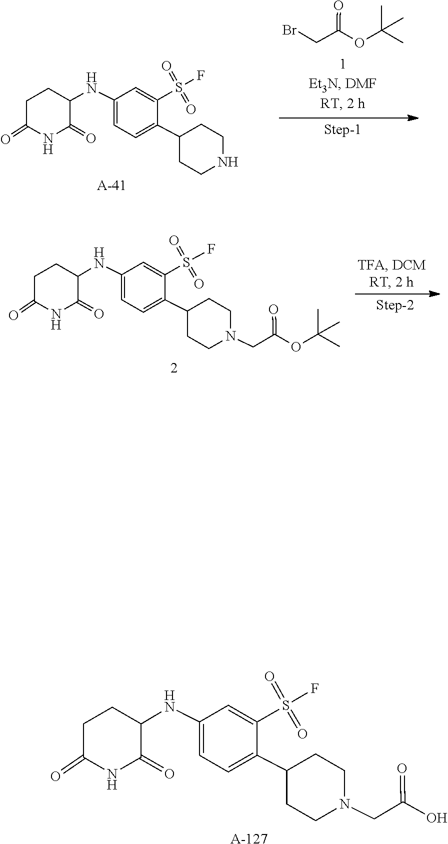

Step-1: tert-butyl 2-(4-(4-((2,6-dioxopiperidin-3-yl)amino)-2-(fluorosulfonyl)phenyl)piperidin-1-yl)acetate To the stirred solution of 5-[(2,6-dioxo-3-piperidyl) amino]-2-(4-piperidyl) benzene sulfonyl fluoride A-41 (490 mg, 1.01 mmol) in dimethylformamide (5.0 mL) was added triethylamine (141.27 L, 1.01 mmol) and tert-butyl 2-bromoacetate 1 (178.38 L, 1.22 mmol) and the reaction mixture stirred at room temperature for 2 hours. After completion, the reaction mixture was concentrated under reduced pressure to obtain the crude product. The crude product was diluted with water (10 mL) and extracted with ethyl acetate (3×15 mL). The combined organic layer was washed with saturated sodium chloride solution (10 mL), dried over anhydrous sodium sulfate and evaporated under reduced pressure to obtain desired product of tert-butyl 2-[4-[4-[(2,6-dioxo-3-piperidyl)amino]-2-fluorosulfonyl-phenyl]-1-piperidyl]acetate 2 (550 mg, 94% yield) as an off-white solid. LCMS (ES*): m z 484.55 [M+H]Step-2: 2-(4-(4-((2,6-dioxopiperidin-3-yl)amino)-2-(fluorosulfonyl)phenyl)piperidin-1-yl)acetic acid (A-127) 10 To a stirred solution of tert-butyl 2-[4-[4-[(2,6-dioxo-3-piperidyl) amino]-2-fluorosulfonyl-phenyl]-1-piperidyl]acetate 2 (550 mg, 1.14 mmol) in 1,2-dichloromethane (5 mL) was added trifluoroacetic acid (870.42 L, 11.37 mmol) and stirred at room temperature for 2 hours. After completion, the reaction mixture was concentrated under reduced pressure to obtain crude product and triturated with diethyl ether (15 mL), dried under reduced pressure to afford 2-[4-[4-[(2,6-dioxo-3-piperidyl)amino]-2-fluorosulfonyl-phenyl]-1-piperidyl]acetic acid (A-127) (430 mg, 63% yield) as an off-white solid. LCMS (ES+): m z 428.55 [M+H]+.

Synthesis A125: Synthesis of 2-(4-(4-(2,6-dioxopiperidin-3-yl)-2-((methylsulfonyl)oxy)phenyl)piperidin-1-yl)acetic acid (A-128) 2 4

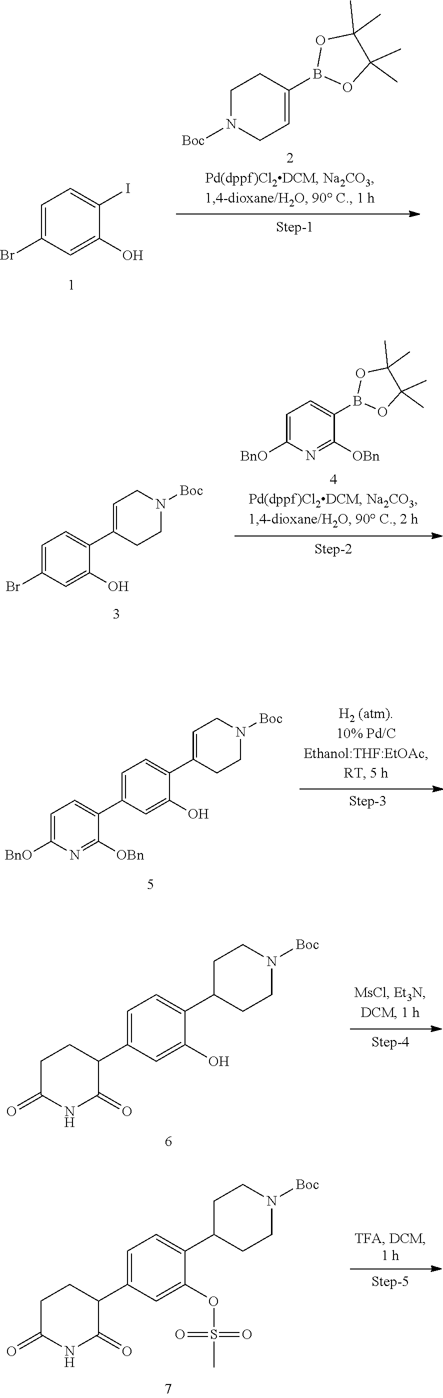

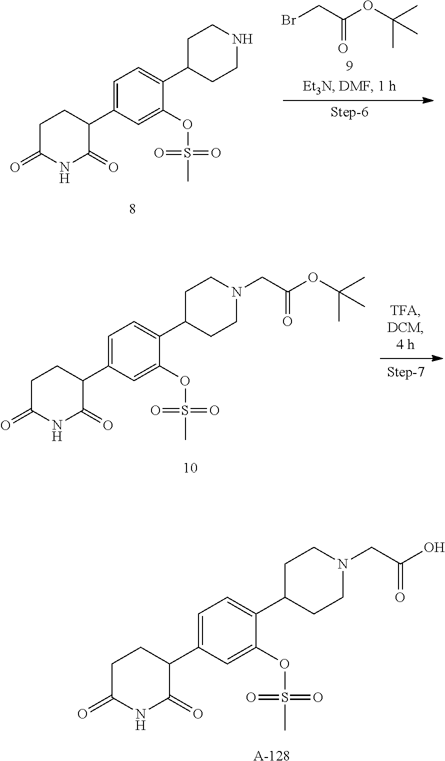

Step-1: Synthesis of tert-butyl 4-(4-bromo-2-hydroxy-phenyl)-3,6-dihydro-2H-pyridine-1-carboxylate To a stirred solution of 5-bromo-2-iodo-phenol 1 (15 g, 50.18 mmol) and tert-butyl 4-(4,4,5,5-tetramethyl-1,3,2-dioxaborolan-2-yl)-3,6-dihydro-2H-pyridine-1-carboxylate 2 (15.52 g, 50.18 mmol), in 1,4-dioxane (120 mL) and water (30 mL) was added sodium carbonate (13.30 g, 125.46 mmol, 5.25 mL) at room temperature. The reaction was degassed 10 with nitrogen for 10 minutes, before adding Pd(dppf)Cl2 DCM complex (3.28 g, 4.01 mmol) and stirred at 70° C. for 1 hour. After completion of the reaction, the reaction mixture was filtered through celite and washed with DCM (200 mL). The filtrate was concentrated under reduced pressure to obtain crude product was purified by column chromatography (using Davisil silica and 15% ethyl acetate in petroleum ether as eluent) to afford tert-butyl 4-(4-bromo-2-hydroxy-phenyl)-3,6-dihydro-2H-pyridine-1-carboxylate 3 (13 g, 61% yield) as a colorless solid. LCMS (ES-): m z 352.12 [M-H]-.

Step-2: Synthesis of tert-butyl 4-[4-(2,6-dibenzyloxy-3-pyridyl)-2-hydroxy-phenyl]-3,6-dihydro-2H-pyridine-1-carboxylate: To a stirred solution of tert-butyl 4-(4-bromo-2-hydroxy-phenyl)-3,6-dihydro-2H-pyridine-1-carboxylate 3 (13 g, 36.70 mmol), 2,6-dibenzyloxy-3-(4,4,5,5-tetramethyl-1,3,2-dioxaborolan-2-yl)pyridine 4 (22.97 mg, 55.05 mmol) in 1,4-dioxane (120 mL) and water (30 mL) was added sodium carbonate (11.67 g, 110.10 mmol) at room temperature. The reaction mixture was degassed with nitrogen for 10 minutes before adding Pd(dppf)C12 DCM complex (2.40 g, 2.94 mmol) and stirred at 60° C. for 1 hour. After completion of the reaction, the reaction mixture was filtered through celite and washed with DCM (200 mL). The filtrate was concentrated under reduced pressure to obtain crude product was purified by column chromatography (using Davisil silica and 20% ethyl acetate in petroleum ether as eluent) to afford tert-butyl 4-[4-(2,6-dibenzyloxy-3-pyridyl)-2-hydroxy-phenyl]-3,6-dihydro-2H-pyridine-1-carboxylate 5 (20 g, 79% yield) as an off-brown solid. LCMS (ES*): m z 565.52 [M+H]+.

Step-3: Synthesis of tert-butyl 4-[4-(2,6-dioxo-3-piperidyl)-2-hydroxy-phenyl]piperidine-1-carboxylate: To a stirred solution of tert-butyl 4-[4-(2,6-dibenzyloxy-3-pyridyl)-2-hydroxy-phenyl]-3,6-dihydro-2H-pyridine-1-carboxylate 5 (20 g, 35.42 mmol) in ethanol (100 mL) and tetrahydrofuran (50 mL) and ethyl acetate (50 mL) was added 10% Pd on carbon (20 g, 187.93 mmol) at room temperature. The reaction mixture was stirred under hydrogen atmosphere (60 psi, Parr shaker) for 5 hours. After completion the reaction, the reaction mixture was filtered through celite bed and washed with ethyl acetate (500 mL). The filtrate was concentrated under reduced pressure to obtain the crude product, which was purified by column chromatography (using Davisil silica and 70% ethyl acetate in petroleum ether as eluent) to afford tert-butyl 4-[4-(2,6-dioxo-3-piperidyl)-2-hydroxy-phenyl]piperidine-1-carboxylate 6 (15 g, 77% yield) as an off-white solid. LCMS (ES-): m z 387.39 [M-100]-.

Step-4: Synthesis of tert-butyl 4-[4-(2,6-dioxo-3-piperidyl)-2-methylsulfonyloxy-phenyl]piperidine-1-carboxylate: To a stirred solution of tert-butyl 4-[4-(2,6-dioxo-3-piperidyl)-2-hydroxy-phenyl]piperidine-1-carboxylate 6 (1.5 g, 3.86 mmol) in DCM (15 mL) was added triethylamine (586.11 mg, 5.79 mmol), followed by methanesulfonyl chloride (530.79 mg, 4.63 mmol, 359.37 L) at 0° C. and stirred at room temperature for 1 hour. After completion of the reaction, the reaction mixture was concentrated under reduced pressure to obtain crude product, which was triturated with n-pentane (25 x mL) and dried under reduced pressure to afford tert-butyl 4-[4-(2,6-dioxo-3-piperidyl)-2-methylsulfonyloxy-phenyl]piperidine-1-carboxylate 7 (3 g, 89% yield) as a brown solid. LCMS (ES-): m z 367.38 [M-Boc]-.

Step-5: Synthesis of [5-(2,6-dioxo-3-piperidyl)-2-(4-piperidyl)phenyl]methanesulfonate: To a stirred solution of tert-butyl 4-[4-(2,6-dioxo-3-piperidyl)-2-methylsulfonyloxy-phenyl]piperidine-1-carboxylate 7 (1.5 g, 3.22 mmol) in DCM (5 mL) was added trifluoroacetic acid (3.67 g, 32.15 mmol, 2.46 mL) at 0° C. and stirred at room temperature for 1 hour. After completion of the reaction, the reaction mixture was concentrated under reduced pressure to obtain the crude product, which was triturated with diethyl ether (20 mL) to afford [5-(2,6-dioxo-3-piperidyl)-2-(4-piperidyl)phenyl]methanesulfonate 8 (1 g, 43% yield, trifluoroacetic acid) as a brown solid. LCMS (ES*): m z 367.34 [M+H]+.

Step-6: Synthesis of tert-butyl 2-[4-[4-(2,6-dioxo-3-piperidyl)-2-methylsulfonyloxy-phenyl]-1-piperidyl]acetate: To a stirred solution of [5-(2,6-dioxo-3-piperidyl)-2-(4-piperidyl)phenyl]methanesulfonate 8 (600 mg, 1.64 mmol, trifluoroacetic acid) in dimethylformamide (4 mL) was added triethylamine (497.07 mg, 4.91 mmol, 684.66 L) followed by addition of tert-butyl 2-bromoacetate 9 (383.26 mg, 1.96 mmol, 288.17 L) at 0° C. and stirred at room temperature for 1 hour. After completion of the reaction, the reaction mixture was diluted with chilled water (10 mL), the precipitated solid was filtered and dried under reduced pressure to afford tert-butyl 2-[4-[4-(2,6-dioxo-3-piperidyl)-2-methylsulfonyloxy-phenyl]-1-piperidyl]acetate 10 (368 mg, 29% yield) as an off-white solid. LCMS (ES-): m z 479.40 [M-H]-.

Step-7: Synthesis of 2-[4-[4-(2,6-dioxo-3-piperidyl)-2-methylsulfonyloxy-phenyl]-1-piperidyl]acetic acid (A-128): To a stirred solution of tert-butyl 2-[4-[4-(2,6-dioxo-3-piperidyl)-2-methylsulfonyloxy-phenyl]-1-piperidyl]acetate 10 (300 mg, 624.25 mol) in DCM (5 mL) added trifluoroacetic acid (711.80 mg, 6.24 mmol, 477.72 L) at 0° C. and stirred at room temperature for 4 hours.

After completion of the reaction, the reaction mixture was concentrated under reduced pressure to obtain crude product, which was triturated with diethyl ether (10 mL) to afford 2-[4-[4-(2,6-dioxo-3-piperidyl)-2-methylsulfonyloxy-phenyl]-1-piperidyl]acetic acid A-128 (288 mg, 67% yield, trifluoroacetic acid) as an off-white solid. LCMS (ES*): m z 425.40 [M+H]+. Synthesis A126: Synthesis of 2-(4-(4-((2,6-dioxopiperidin-3-yl)amino)-2-((methylsulfonyl)oxy)phenyl)piperidin-1-yl)acetic acid (A-129)

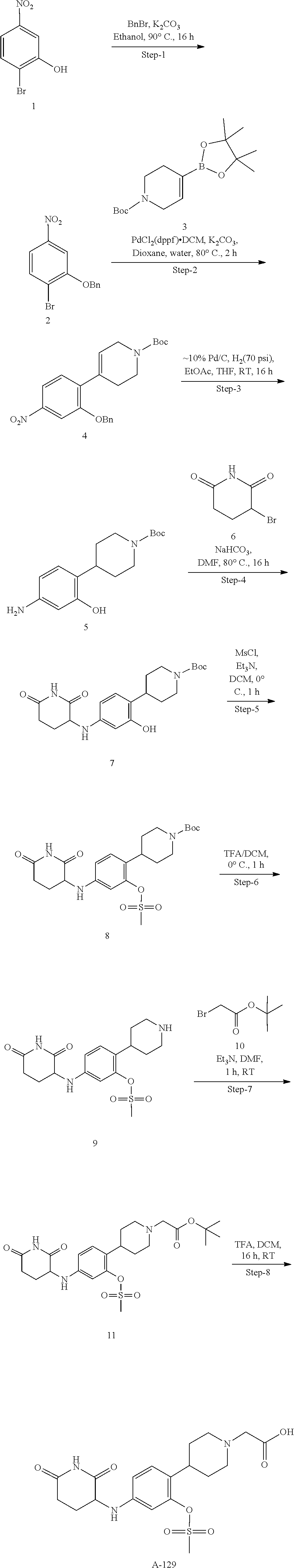

Step-i: Synthesis of 2-benzyloxy-1-bromo-4-nitro-benzene: To the stirred mixture of 2-bromo-5-nitro-phenol 1 (5 g, 22.94 mmol) in ethanol 10 (206.07 mL) were added potassium carbonate (3.80 g, 27.52 mmol), potassium iodide (2.45 g, 14.78 mmol, and 786.56 tL) and benzyl bromide (5.10 g, 29.82 mmol, and 3.55 mL) at room temperature. The reaction mixture was stirred at 90° C. for 16 hours. After completion of the reaction, the reaction mixture was concentered under reduced pressure to obtain the crude product, which was diluted with chilled water (50 mL), the solid precipitate obtained was filtered and dried under reduced pressure to afford 2-benzyloxy-1-bromo-4-nitro-benzene 2 (4.8 g, 65% yield) as an off-white solid. H-NMR (400 MHz, DMSO-d6): δ 7.800 (s, 1H), 7.76 (s, 2H), 7.50 (d, J=7.2 Hz, 2H), 7.44 (t, J=6.8 Hz, 2H), 7.36 (d, J=7.2 Hz, 1H), 5.25 (s, 2H).

Step-2: Synthesis of tert-butyl 4-(2-benzyloxy-4-nitro-phenyl)-3,6-dihydro-2H-pyridine-1-carboxylate: To a stirred solution of 2-benzyloxy-1-bromo-4-nitro-benzene 2 (2 g, 6.49 mmol) and tert-butyl 4-(4,4,5,5-tetramethyl-1,3,2-dioxaborolan-2-yl)-3,6-dihydro-2H-pyridine-1-carboxylate 3 (2.21 g, 7.14 mmol) in 1,4-dioxane (20 mL) and water (6 mL) was added 10 potassium carbonate (2.69 g, 19.47 mmol) at room temperature. The reaction mixture was degassed with argon gas for 10 minutes before adding Pd(dppf)C12 DCM complex (424.05 mg, 519.27 mol) and stirred at 80° C. for 2 hours. After completion of the reaction, the reaction mixture was filtered through celite bed and washed with ethyl acetate (20 mL), the filtrate was concentrated under reduced pressure to get crude, which was purified by column 15 chromatography (using silica gel 230-400 mesh and 10% ethyl acetate in petroleum ether as eluent) to afford tert-butyl 4-(2-benzyloxy-4-nitro-phenyl)-3,6-dihydro-2H-pyridine-1-carboxylate 4 (1.3 g, 42% yield) as yellow-color liquid. LC-MS (ES): m z 311.35 [M-Boc]*.

Step-3: Synthesis of tert-butyl 4-(4-amino-2-hydroxy-phenyl) piperidine-1-carboxylate: To a stirred solution of tert-butyl 4-(2-benzyloxy-4-nitro-phenyl)-3, 6-dihydro-2H-pyridine-1-carboxylate 4 (4 g, 9.75 mmol) in tetrahydrofuran (20 mL) and ethyl acetate (20 mL) was added 10% palladium on carbon (4.00 g, 37.59 mmol) at room temperature. The reaction mixture was stirred under hydrogen atmosphere (70 psi, Parr shaker) at room temperature for 16 hours. After completion of the reaction, the reaction mixture was filtered through a celite bed and washed with DCM (20 mL). The filtrate was concentrated under reduced pressure to get crude tert-butyl 4-(4-amino-2-hydroxy-phenyl) piperidine-1-carboxylate 5 (2.5 g, 68% yield) as a brown solid. LC-MS (ES-): m z 291.20 [M-H]-.

Step-4: Synthesis of tert-butyl 4-[4-[(2,6-dioxo-3-piperidyl)amino]-2-hydroxy-phenyl]piperidine-1-carboxylate: To a stirred solution of tert-butyl 4-(4-amino-2-hydroxy-phenyl) piperidine-1-carboxylate 5 (2.5 g, 8.55 mmol) in dimethylformamide (6.43 mL) was added N,N-diisopropylethylamine (3.32 g, 25.65 mmol, and 4.47 mL) and stirred for 10 minutes before adding 3-bromopiperidine-2,6-dione 6 (2.46 g, 12.83 mmol) and stirred at 80° C. for 16 hours.

After completion of the reaction, the reaction mixture was concentrated under reduced pressure to obtain the crude product, which was diluted with chilled water (20 mL), the solid precipitate obtained was filtered and dried to get tert-butyl 4-[4-[(2,6-dioxo-3-piperidyl)amino]-2-hydroxy-phenyl]piperidine-1-carboxylate 7 (1.2 g, 23% yield) as an off-white solid. LC-MS (ES-): m z 402.44 [M-H]-.

Step-5: Synthesis of tert-butyl 4-[4-[(2,6-dioxo-3-piperidyl)amino]-2-methylsulfonyloxy-phenyl]piperidine-1-carboxylate: To a stirred solution of tert-butyl 4-[4-[(2,6-dioxo-3-piperidyl)amino]-2-hydroxy-phenyl]piperidine-1-carboxylate 7 (0.6 g, 1.49 mmol) in DCM (6 mL) were added triethylamine (300.96 mg, 2.97 mmol, 414.54 L), followed by methane sulfonyl chloride (204.42 mg, 1.78 mmol, 138.40 L) at 0° C. The reaction mixture was stirred at room temperature for 1 hour. After completion of the reaction, the reaction mixture was diluted with water (10 mL) and extracted with DCM (2×20 mL). The combined organic layer was dried over sodium sulfate and concentrated under reduced pressure to get the crude product. Which was purified by prep-HPLC to afford tert-butyl 4-[4-[(2,6-dioxo-3-piperidyl)amino]-2-methylsulfonyloxy-phenyl]piperidine-1-carboxylate 8 (0.3 g, 40% yield) as a pale-yellow solid. Prep HPLC Method: Column: X-SELECT C18, 5 pm (100mmX19 mm), Mobile Phase A: 0.1% FA in water, B: acetonitrile, Gradient (T % B):0/10, 2/15, 14/95, Flow Rate: 18 ml/min, Sample Diluent: acetonitrile+water+tetrahydrofuran. LC-MS (ES-): m z 480.14 [M-H]-.

Step-6: Synthesis of [5-[(2,6-dioxo-3-piperidyl)amino]-2-(4-piperidyl)phenyl]methane sulfonate: To a stirred solution of tert-butyl 4-[4-[(2,6-dioxo-3-piperidyl)amino]-2-methylsulfonyloxy-phenyl]piperidine-1-carboxylate 8 (0.13 g, 269.96 mol) in DCM (3.33 mL) was added trifluoroacetic acid (369.37 mg, 3.24 mmol, 247.90 L) at 0° C. The reaction mixture was stirred at room temperature for 1 hour. After completion of the reaction, the reaction mixture was concentrated under reduced pressure to get crude product, which was triturated with diethyl ether (20 mL) to afford [5-[(2,6-dioxo-3-piperidyl)amino]-2-(4-piperidyl)phenyl]methane sulfonate 9 (0.13 g, 220.76 mol, 82% yield, TFA salt) as a green solid. LCMS (ES*): m z 382.48 [M+H]+.

Step-7: Synthesis of tert-butyl 2-[4-[4-[(2,6-dioxo-3-piperidyl)amino]-2-methylsulfonyloxy-phenyl]-1-piperidyl]acetate: To a stirred solution of [5-[(2,6-dioxo-3-piperidyl)amino]-2-(4-piperidyl)phenyl]methane sulfonate 9 (0.13 g, 340.81 mol) in dimethylformamide (1.53 mL) was added triethylamine (103.46 mg, 1.02 mmol, 142.50 L) at room temperature and stirred for 5 minutes, before adding tert-butyl 2-bromoacetate 10 (33.24 mg, 170.40 mol, 24.99 pL) and stirred at room temperature for 1 hour. After completion of the reaction, the reaction mixture was diluted with chilled water (10 mL), the solid precipitate obtained was filtered and dried under reduced pressure to afford tert-butyl 2-[4-[4-[(2,6-dioxo-3-piperidyl)amino]-2-methylsulfonyloxy-phenyl]-1-piperidyl]acetate 11 (0.1 g, 146.07 mol, 43% yield) as a green solid. LC-MS (ES-): m z 494.31 [M-H]-. 5 Step-8: Synthesis of 2-[4-[4-[(2,6-dioxo-3-piperidyl)amino]-2-methylsulfonyloxy-phenyl]-1-piperidyl]acetic acid (A-129): To a stirred solution of tert-butyl 2-[4-[4-[(2,6-dioxo-3-piperidyl)amino]-2-methylsulfonyloxy-phenyl]-1-piperidyl]acetate 11 (0.1 g, 201.78 mol) in DCM (2 mL) was added trifluoroacetic acid (276.09 mg, 2.42 mmol, 185.30 L) at 0° C. The reaction mixture 10 was stirred at room temperature for 16 hours. After completion of the reaction, the reaction mixture was concentrated under reduced pressure to get crude product. Which was triturated with diethyl ether (10 mL) to afford 2-[4-[4-[(2,6-dioxo-3-piperidyl)amino]-2-methylsulfonyloxy-phenyl]-1-piperidyl]acetic acid A-129 (0.085 g, 77% yield) as a pale green solid. LC-MS (ES-): m z 440.44 [M+H]+.

Synthesis A127: Synthesis of 2-(4-(4-((2,6-dioxopiperidin-3-yl)oxy)-2-((methylsulfonyl)oxy)phenyl)piperidin-1-yl)acetic acid (A-130):

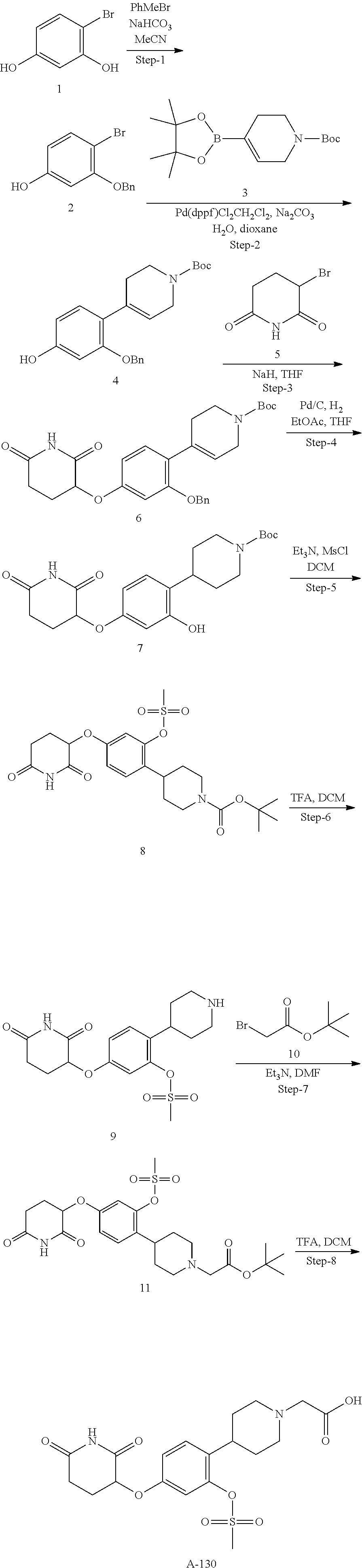

Step-1:

To a stirred solution of 4-bromobenzene-1,3-diol 1 (25 g, 132.27 mmol) in acetonitrile (250.00 mL) were added bromomethylbenzene (22.62 g, 132.27 mmol, 15.73 mL), sodium 5 bicarbonate (22.22 g, 264.54 mmol) and stirred at 90° C. for 16 h. After completion of the reaction, the reaction mixture was filtered through a pad of celite. The filtrate was concentrated under reduced pressure to give the crude product, which was purified by column chromatography (Davisil silica and 7% ethyl acetate in pet ether as eluent) to afford 3-benzyloxy-4-bromo-phenol 2 (15 g, 37.56 mmol, 28.40% yield) as a colorless gum. LC-MS 10 (ES-): m z 277.06 [M - H]-Step-2: To a stirred solution of 3-benzyloxy-4-bromo-phenol 2 (15 g, 53.74 mmol) and tert-butyl 4-(4,4,5,5-tetramethyl-1,3,2-dioxaborolan-2-yl)-3,6-dihydro-2H-pyridine-1-carboxylate 3 (19.94 g, 64.49 mmol) in water (40.00 mL) and dioxane (110.00 mL) was added sodium 15 carbonate (17.09 g, 161.22 mmol). The reaction mixture was degassed with argon gas for 10 min before addition of bis(1-diphenylphosphanylcyclopenta-2,4-dien-1-yl)iron;dichloromethane;dichloropalladium (4.39 g, 5.37 mmol) and stirred at 100° C. for 6 h.

After completion of the reaction, the reaction mixture was filtered through a pad of celite and washed with ethyl acetate (400 mL). The filtrate was concentrated under reduced 20 pressure to give the crude product, which was purified by column chromatography (Davisil silica and 20% ethyl acetate in pet ether as eluent) to afford tert-butyl 4-(2-benzyloxy-4-hydroxy-phenyl)-3,6-dihydro-2H-pyridine-1-carboxylate 4 (16 g, 35.23 mmol, 65.56% yield) as a light yellow solid. LC-MS (ES-): m z 380.41 [M - H]-Step-3: To a stirred solution of tert-butyl 4-(2-benzyloxy-4-hydroxy-phenyl)-3,6-dihydro-2H-pyridine-1-carboxylate 4 (16 g, 41.94 mmol) in THF (160 mL) was added sodium hydride (60% dispersion in mineral oil) (2.52 g, 104.86 mmol) portionwise and stirred at 0° C. for 40 min. Then 3-bromopiperidine-2,6-dione 5 (16.11 g, 83.89 mmol) in THF (160 mL) was added dropwise into the reaction at 0° C. and stirred at 50° C. for 1 h. After completion of the 30 reaction, the reaction mixture was quenched with saturated ammonium chloride solution (150 mL), diluted with water (100 mL) and extracted with ethyl acetate (250 mL×2). The combined organic layer was dried over anhydrous sodium sulfate and concentrated under reduced pressure to give the crude product, which was purified by column chromatography (Davisil silica and 40% ethyl acetate in pet ether as eluent) to afford tert-butyl 4-[2-benzyloxy-4-[(2,6-dioxo-3-piperidyl)oxy]phenyl]-3,6-dihydro-2H-pyridine-1-carboxylate 6 (13 g, 19.79 mmol, 47.19% yield) as a colorless gum. LC-MS (ES-): m z 491.48 [M - H]-Step-4: To a stirred solution of tert-butyl 4-[2-benzyloxy-4-[(2,6-dioxo-3-piperidyl)oxy]phenyl]-3,6-dihydro-2H-pyridine-1-carboxylate 6 (13 g, 26.39 mmol) in ethyl acetate (150 mL) and THF (150 mL) was added 10% palladium on carbon (wetted with ca. 55% water) (10 g, 93.97 mmol) at room temperature. The reaction mixture was stirred under hydrogen atmosphere (60 psi, parr shaker) at room temperature for 4 h. Upon completion of the reaction, the reaction mixture was filtered through a pad of celite, which was subsequently washed with ethyl acetate (300 mL).The filtrate was concentrated and dried under vacuum to give the crude product, which was purified by column chromatography (Davisil silica and 70% ethyl acetate in pet ether as eluent) to afford tert-butyl 4-[4-[(2,6-dioxo-3-piperidyl)oxy]-2-hydroxy-phenyl]piperidine-1-carboxylate 7 (7 g, 13.04 mmol, 49.40% yield) as an off-white solid. LC-MS (ES-): m z 403.22 [M - H]-Step-5: To a stirred solution of tert-butyl 4-[4-[(2,6-dioxo-3-piperidyl)oxy]-2-hydroxy-phenyl]piperidine-1-carboxylate 7 (2 g, 4.94 mmol) in dichloromethane (30 mL) added triethylamine (1.50 g, 14.83 mmol, 2.07 mL) and methanesulfonyl chloride, 98% (1.48 g, 12.91 mmol, 1.00 mL) at 0° C. and the resulting reaction mixture was stirred at 0° C. for 2 h. Upon completion of the reaction, the reaction mixture was concentrated under reduced pressure to give the crude product, which was triturated with n-pentane (50 mL×2) and dried under reduced pressure to afford tert-butyl 4-[4-[(2,6-dioxo-3-piperidyl)oxy]-2-methylsulfonyloxy-phenyl]piperidine-1-carboxylate 8 (2 g, 29.64% yield) as a yellow gum. LC-MS (ES-): m z 481.39 [M - H]-Step-6: To a stirred solution of tert-butyl 4-[4-[(2,6-dioxo-3-piperidyl)oxy]-2-methylsulfonyloxy-phenyl]piperidine-1-carboxylate 8 (2 g, 4.14 mmol) in dichloromethane (15.31 mL) was added trifluoracetic acid (14.44 g, 126.61 mmol, 9.69 mL) at 0° C., and the reaction mixture was stirred at room temperature for 2 h. Upon completion of the reaction, the reaction mixture concentrated under reduced pressure and the residue was triturated with diethyl ether (50 mL×3) to afford [5-[(2,6-dioxo-3-piperidyl)oxy]-2-(4-piperidyl)phenyl]methanesulfonate 9 (1.7 g, 3.14 mmol, 75.71% yield) as an off-white solid. LC-MS (ES+): m z 383.42 [M+H]+Step-7: To a stirred solution of [5-[(2,6-dioxo-3-piperidyl)oxy]-2-(4-piperidyl)phenyl]methanesulfonate 9 (0.7 g, 1.83 mmol) in DMF (2.49 mL) was added triethylamine (555.65 mg, 5.49 mmol, 765.36 L) followed by tert-butyl 2-bromoacetate 10 (357.03 mg, 1.83 mmol, 268.44 pL) at 0° C. and the reaction mixture was stirred at room temperature for 1 h. After completion of the reaction, the reaction mixture was diluted with chilled water (10 mL). The 10 obtained precipitate was filtered and dried under reduced pressure to afford tert-butyl 2-[4-[4-[(2,6-dioxo-3-piperidyl)oxy]-2-methylsulfonyloxy-phenyl]-1-piperidyl]acetate 11 (0.5 g, 25.96% yield) as a yellow solid. LC-MS (ES+): m z 497.42 [M+H]+Step-8: To a stirred solution of tert-butyl 2-[4-[4-[(2,6-dioxo-3-piperidyl)oxy]-2-methylsulfonyloxy-phenyl]-1-piperidyl]acetate 11 (0.5 g, 1.01 mmol) in DCM (4 mL) was added TFA (574.05 mg, 5.03 mmol, 385.27 L) at 0° C. and the reaction was stirred at room temperature for 6 h. After completion of the reaction, the reaction mixture was concentrated under reduced pressure to give the crude product, which was purified by Prep HPLC to afford 2-[4-[4-[(2,6-dioxo-3-piperidyl)oxy]-2-methylsulfonyloxy-phenyl]-1-piperidyl]acetic acid A-130 (130 mg, 28.92% yield) as an off-white solid. LC-MS (ES+): m/z 441.29 [M+H]+. H-NMR (400 MHz, DMSO-d6): δ 10.97 (s, 1H), 7.33 (d, J=8.4 Hz, 1H), 7.06-7.01 (m, 2H), 5.26-5.22 (m, 1H), 3.49 (s, 3H), 3.39 (s, 2H), 3.28 (d, J=11.6 Hz, 2H), 2.92 (t, J=12.0 Hz, 1H), 2.76-2.63 (m, 4H), 2.24-2.17 (m, 2H), 1.87-1.81 (m, 2H), 1.76-1.70 (m, 2H) ppm. Prep HPLC Method: Column/dimensions: Sunfire (19*150*5 p); Mobile phase A: 0.1% FA in Water; Mobile Phase B: 100% Acetonitrile; Gradient (Time/% B): 0/5, 2/5, 15/55, 15.10/98, 17.50/5.20/5; Flow rate: 18 mL/min; Solubility: Water+Acetonitrile Synthesis A128: Synthesis of 3-[4-(2-fluorosulfonyloxyphenyl)triazol-1-yl]-2,6-dioxo-piperidine (A-131)

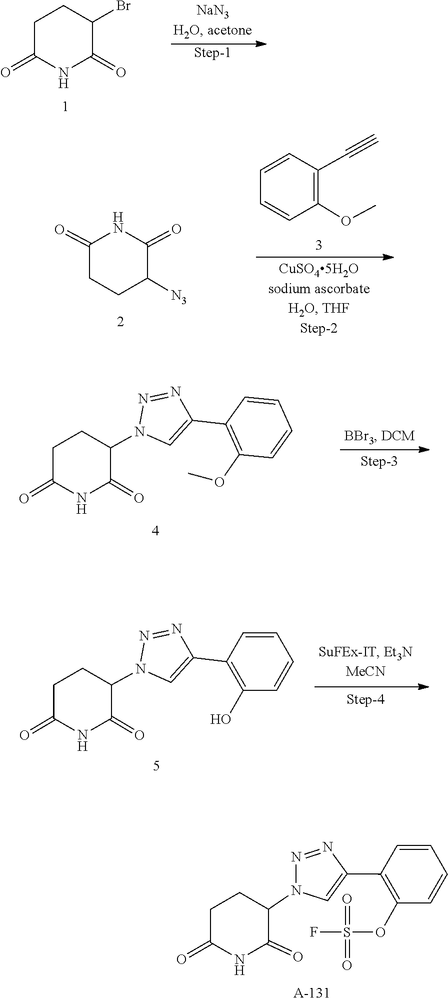

5 Step-1: 3-azidopiperidine-2, 6-dione To a stirred solution of 3-bromopiperidine-2, 6-dione 1 (3 g, 15.62 mmol) in acetone (25 mL) and water (13 mL) was added sodium azide (5.08 g, 78.12 mmol) at 0° C. and stirred at room temperature for 16 h. After completion of the reaction, the reaction mixture was concentrated under reduced pressure and the obtained residue was diluted with water (50 mL) 10 and extracted with ethyl acetate (20 mL×3). The combined organic layer was dried over anhydrous sodium sulfate and concentrated under reduced pressure to afford 3-azidopiperidine-2, 6-dione 2 (1.5 g, 58.53% yield) as an off-white solid. LC-MS (ES*): m z 154.80 [M+H]+.

Step-2: 3-[4-(2-methoxyphenyl) triazol-1-yl]piperidine-2, 6-dione To a stirred solution of 3-azidopiperidine-2,6-dione 2 (0.5 g, 3.24 mmol), 1-ethynyl-2-methoxy-benzene 3 (472 mg, 3.57 mmol) in THF (6 mL) and water (2 mL) were added sodium ascorbate (257 mg, 1.30 mmol), copper sulfate pentahydrate (81 mg, 0.324 mmol) and stirred for 16 h at room temperature. After completion of the reaction, the reaction mixture was filtered through a pad of celite, which was subsequently washed with ethyl acetate (30 mL). The filtrate 20 was diluted with water (10 mL) and extracted with ethyl acetate (10 mL×2). The combined organic layer was dried over anhydrous sodium sulfate and concentrated under reduced pressure to afford 3-[4-(2-methoxyphenyl) triazol-1-yl]piperidine-2, 6-dione 4 (0.2 g, 19% yield) as a pale brown solid. LC-MS (ES+): m z 287.00 [M+H]+.

Step-3: 3-[4-(2-hydroxyphenyl)triazol-1-yl]piperidine-2,6-dione To a stirred solution of 3-[4-(2-methoxyphenyl)triazol-1-yl]piperidine-2,6-dione 4 (0.2 g, 0.698 mmol) in DCM (2 mL) was added tribromoborane (0.33 mL, 3.49 mmol) at −78° C. and stirred at room temperature for 16 h. After completion of the reaction, the reaction mixture was quenched with ice-cold water(10 mL) and extracted with DCM (10 mL×2). The organic layer was dried over anhydrous sodium sulfate and concentrated under reduced pressure to afford 3-[4-(2-hydroxyphenyl)triazol-1-yl]piperidine-2,6-dione 5 (0.15 g, 70.07% yield) as a pale yellow gummy. LC-MS (ES*): m z 273.00 [M+H]+.

Step-4: 3-[4-(2-fluorosulfonyloxyphenyl)triazol-1-yl]-2,6-dioxo-piperidine To a stirred solution of 3-[4-(2-hydroxyphenyl)triazol-1-yl]piperidine-2,6-dione 5 (0.15 g, 550.95 mol) in Acetonitrile (3 mL) was added triethylamine (0.154 mL, 1.10 mmol) at room temperature and stirred for 5 min. 1-(fluorosulfonyl)-2,3-dimethyl-1H-imidazol-3-ium trifluoromethanesulfonate (272.11 mg, 826.42 mol) was added into the reaction mixture at 0° C. and stirred for 1 h at room temperature. After completion of the reaction, the reaction mixture was concentrated under reduced pressure to afford crude product.

The crude product was purified by MS Prep HPLC to afford 3-[4-(2-fluorosulfonyloxyphenyl)triazol-1-yl]-2,6-dioxo-piperidineA-131 (72.8 mg, 36.79% yield) as an off-white solid. LC-MS (ES+): m z 355.15 [M+H]+. ¹H NMR (400 MHz, DMSO-d6): δ 11.27 (s, 1H), 8.70 (s, 1H), 8.10-8.08 (m, 1H), 7.73 (d, J=8.0 Hz, 1H), 7.66-7.58 (m, 2H), 5.93-5.89 (m, 1H), 2.94-2.85 (m, 1H), 2.79-2.75 (m, 2H), 2.39-2.33 (m, 1H) ppm. 9F NMR (376 MHz, DMSO-d6): δ 42.78 ppm.

Synthesis A129: Synthesis of 2-(4-(4-(2,4-dioxotetrahydropyrimidin-1(2H)-yl)-2-((methylsulfonyl)oxy)phenyl)piperidin-1-yl)acetic acid (A-132) Boc

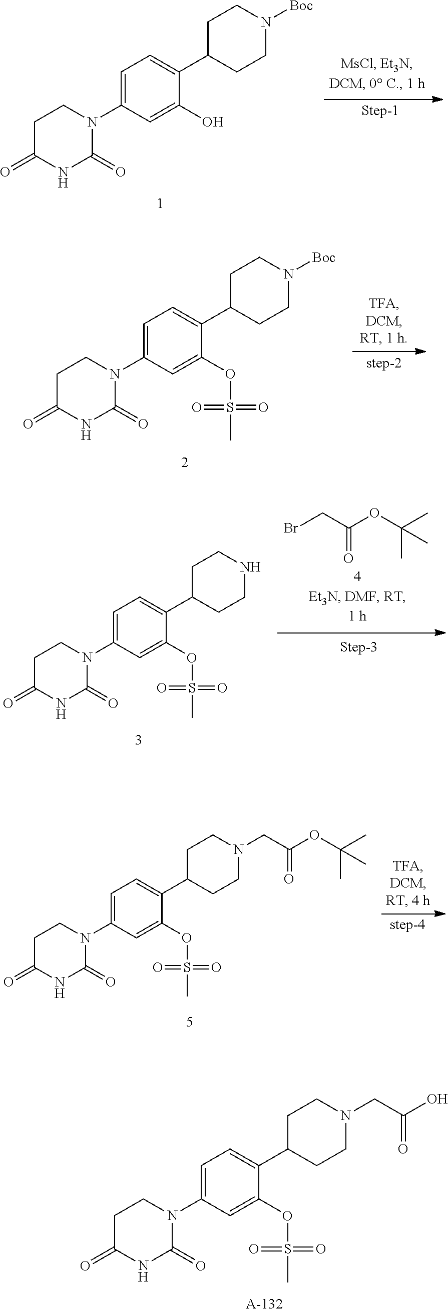

Step-1: Synthesis of tert-butyl 4-[4-(2,4-dioxohexahydropyrimidin-1-yl)-2-methylsulfonyloxy-phenyl]piperidine-1-carboxylate To a stirred solution of tert-butyl 4-[4-(2,4-dioxohexahydropyrimidin-1-yl)-2-hydroxy-phenyl]piperidine-1-carboxylate 1 (1 g, 2.57 mmol) and methanesulfonyl chloride (352.96 mg, 3.08 mmol, 238.97 L) in DCM (20 mL) was added triethylamine (389.75 mg, 3.85 mmol, 536.84 L) and stirred for 1 hour at 0° C. After completion, the reaction mixture was concentrated under reduced pressure to get crude product, which was triturated with n-pentane (2×20 mL) and dried under high vacuum to afford tert-butyl 4-[4-(2,4-dioxohexahydropyrimidin-1-yl)-2-methylsulfonyloxy-phenyl]piperidine-1-carboxylate 2 (1 g, 65.80% yield) as a pale-yellow solid. LC-MS (ES-): m/z 466.37 [M-H]-.

Step-2: Synthesis of [5-(2,4-dioxohexahydropyrimidin-1-yl)-2-(4-piperidyl)phenyl]methanesulfonate To a stirred solution of tert-butyl 4-[4-(2,4-dioxohexahydropyrimidin-1-yl)-2-methylsulfonyloxy-phenyl]piperidine-1-carboxylate 2 (1 g, 2.14 mmol) in DCM (30 mL) was added trifluoroacetic acid (7.45 g, 65.34 mmol, 5 mL) at 0° C. and the reaction mixture was stirred at room temperature for 2 hours. The reaction mixture evaporated under reduced pressure and triturated with diethyl ether (50 mL) to afford [5-(2,4-dioxohexahydropyrimidin-1-yl)-2-(4-piperidyl)phenyl]methanesulfonate 3 (1 g, 95.44% yield) as an off-white solid. LC-MS (ES*): m z 368.33 [M+H]+.

Step-3: Synthesis of tert-butyl 2-[4-[4-(2,4-dioxohexahydropyrimidin-1-yl)-2-methylsulfonyloxy-phenyl]-1-piperidyl]acetate To a stirred solution of [5-(2,4-dioxohexahydropyrimidin-1-yl)-2-(4-piperidyl)phenyl]methanesulfonate 3 (1 g, 2.08 mmol, Trifluoroacetic acid) in N,N-Dimethylformamide (10 mL) added triethylamine (630.54 mg, 6.23 mmol, 868.52 L) and tert-butyl 2-bromoacetate 4 (486.17 mg, 2.49 mmol, 365.54 L) at 0° C. and stirred at room temperature for 1 hour. After completion of the reaction, the reaction mixture diluted with ice-cold water (10 mL). The obtained solid was collected and dried under reduced pressure to afford tert-butyl 2-[4-[4-(2,4-dioxohexahydropyrimidin-1-yl)-2-methylsulfonyloxy-phenyl]-1-piperidyl]acetate 5 (0.65 g, 47% yield) as an off-white solid. LC-MS (ES-): m z 480.23 [M-H]-.

Step-4: Synthesis of 2-[4-[4-(2,4-dioxohexahydropyrimidin-1-yl)-2-methylsulfonyloxy-phenyl]-1-piperidyl]acetic acid To a stirred solution of tert-butyl 2-[4-[4-(2,4-dioxohexahydropyrimidin-1-yl)-2-methylsulfonyloxy-phenyl]-1-piperidyl]acetate 5 (0.3 g, 622.97 mol) in dichloromethane (20 mL) added trifluoroacetic acid (1.42 g, 12.46 mmol, 953.44 L) at 0° C. and stirred at room temperature for 4 hours. After completion of the reaction, the reaction mixture was evaporated under reduced pressure and triturated with diethyl ether (2×50 mL) to afford 2-[4-[4-(2,4-dioxohexahydropyrimidin-1-yl)-2-methylsulfonyloxy-phenyl]-1-piperidyl]acetic acid A-132 (0.150 g, 53% yield) as an off-white solid. LC-MS (ES*): m z 426 [M+H]+. ¹H NMR (400 MHz, DMSO-d6): δ 13.99 (s, 1H), 10.47 (s, 1H), 7.40 (s, 3H), 4.12-0.00 (m, 2H), 3.82 (t, J=13.20 Hz, 2H), 3.50 (m, 5H), 3.23 (m, 3H), 2.73 (t, J=6.80 Hz, 2H), 2.09-1.91 (m, 4H) 15 Synthesis A128: Synthesis of 2-(1-methyl-2,6-dioxopiperidin-3-yl)-3-oxoisoindoline-5-sulfonyl fluoride (A-133) NH₂

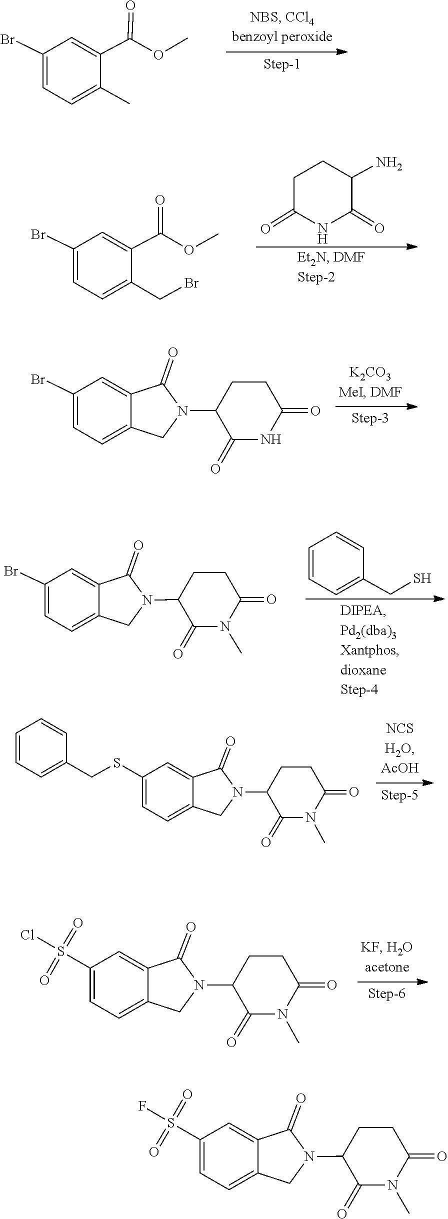

To a stirred solution of methyl 5-bromo-2-methyl-benzoate (2 g, 8.73 mmol) in CCl₄ (40 mL) were added N-bromosuccinimide, 99% (1.71 g, 9.60 mmol) and benzoyl peroxide (211.49 mg, 873.09 mol) and the reaction mixture was stirred at 90° C. for 16 h. After completion of the reaction, the reaction mixture was quenched with water (50 ml) followed by extraction with DCM (2×50 ml). The organic layer was dried over sodium sulfate, filtered and concentrated under reduced pressure. The crude was purified by CombiFlash® chromatography (Davisil silica) to afford methyl 5-bromo-2-(bromomethyl)benzoate (1.50 g, 4.63 mmol, 53.00% yield) as a yellow oil. H-NMR (400 MHz, CDCl3) δ 8.11-8.10 (m, 1H), 7.62 (dd, J=8.2, 2.2 Hz, 1H), 7.34 (d, J=8.0 Hz, 1H), 4.90 (s, 2H), 3.95 (s, 3H) ppm. Step-2: To a stirred solution of methyl 5-bromo-2-(bromomethyl)benzoate (1.50 g, 4.87 mmol) and 3-aminopiperidine-2,6-dione (936.11 mg, 7.31 mmol) in DMF (10 mL) was added triethylamine (4.93 g, 48.71 mmol, 6.79 mL) and the reaction mixture was stirred at 90° C. for 16 h. After completion of the reaction, the reaction mixture was quenched with ice cold water. The obtained precipitate was filtered and dried under vacuum to afford 3-(6-bromo-1-oxo-isoindolin-2-yl)piperidine-2,6-dione (600 mg, 1.65 mmol, 33.93% yield) as a violet solid.

LCMS (ES+): m z 323.04 [M+H]+Step-3: To a stirred solution of 3-(6-bromo-1-oxo-isoindolin-2-yl)piperidine-2,6-dione (600 mg, 1.86 mmol) in DMF (7 mL) was added potassium carbonate (769.85 mg, 5.57 mmol) followed by iodomethane (395.32 mg, 2.79 mmol, 173.39 L) at room temperature. The reaction mixture was stirred at room temperature for 16 h. After completion of the reaction, the reaction mixture was quenched with ice cold water (45 mL), and extracted with ethyl acetate (2×50 ml). The organic layer was dried over anhydrous sodium sulfate, filtered and concentrated under reduced pressure. The crude product was purified by CombiFlash® (Davisil silica, 45% ethyl acetate in pet ether) to afford 3-(6-bromo-1-oxo-isoindolin-2-yl)-1-methyl-piperidine-2,6-dione (355 mg, 897.38 mol, 48.33% yield) as a gray solid. LCMS (ES+): m z 339.05 [M+H]+Step-4: To a stirred solution 3-(6-bromo-1-oxo-isoindolin-2-yl)-1-methyl-piperidine-2,6-dione (350 mg, 1.04 mmol) and phenylmethanethiol (257.86 mg, 2.08 mmol) in 1,4-dioxane (10 ml) was added DIPEA (402.48 mg, 3.11 mmol, 542.43 L). The mixture purged with N2 for 10 min before Pd2(dba)3 (95.06 mg, 103.81 mol) and (5-diphenylphosphanyl-9,9-dimethyl-xanthen-4-yl)-diphenyl-phosphane (120.13 mg, 207.61 mol) was added and stirred at 120° C. for 16 h. After completion of the reaction, the reaction mixture was quenched with water (30 ml) and extracted with ethyl acetate (2×30 ml). The organic layer was dried over anhydrous sodium sulfate, filtered and concentrated under reduced pressure. The crude product was purified by column chromatography (Davisil silica gel, 40% to 55% ethyl acetate in pet ether) to afford 3-(6-benzylsulfanyl-1-oxo-isoindolin-2-yl)-1-methyl-piperidine-2,6-dione (300 mg, 756.59 mol, 72.88% yield) as an off-white solid. LCMS (ES*): m z 381.32 [M+H]+Step-5: To a stirred solution of 3-(6-benzylsulfanyl-1-oxo-isoindolin-2-yl)-1-methyl-piperidine-2,6-dione (300 mg, 788.52 mol) in water (3 mL) and acetic acid (12 mL) was added N-chlorosuccinimide (315.88 mg, 2.37 mmol) at room temperature and stirred for 2 h.

After completion of the reaction, the reaction mixture was quenched with water (30 ml) and extracted with EtOAc (30 ml). The organic layer was dried over anhydrous sodium sulfate, filtered and concentrated under reduced pressure to give 2-(1-methyl-2,6-dioxo-3-piperidyl)-3-oxo-isoindoline-5-sulfonyl chloride (300 mg, 681.09 mol, 86.38% yield). LCMS (ES*): m z 357.09 [M+H]+Step-6: To a stirred solution of 2-(1-methyl-2,6-dioxo-3-piperidyl)-3-oxo-isoindoline-5-sulfonyl chloride (300 mg, 840.85 mol) in water (10 mL) and acetone (10 mL) was added potassium fluoride (97.70 mg, 1.68 mmol) at room temperature and stirred for 30 min.

After completion of the reaction, the reaction mixture was diluted with EtOAc (30 ml) and water (30 ml). The organic layer was dried over anhydrous sodium sulfate, filtered and concentrated under reduced pressure. The crude was purified by reverse phase HPLC to afford 2-(1-methyl-2,6-dioxo-3-piperidyl)-3-oxo-isoindoline-5-sulfonyl fluoride (132 mg, 370.68 mol, 44.08% yield) as an off-white solid. LCMS (ES*): m z 339.18 [M - H]-. H-NMR (400 MHz, DMSO-d6): δ 8.41 (dd, J=8.0, 1.6 Hz, 1H), 8.34 (s, 1H), 8.07 (d, J=8.0 Hz, 1H), 5.27-5.23 (m, 1H), 4.72-4.53 (m, 2H), 3.03-2.96 (m, 4H), 2.80-2.76 (m, 1H), 2.45-2.37 (m, 1H), 2.09-2.07 (m, 1H) ppm. Prep HPLC Method: Column/dimensions: X-bridge 19*150, Sum; Mobile phase A: 0.1% formic acid in water; Mobile phase B: 100% ACN Gradient; (Time/% B):0.1/25,3/25,7/50,12/50,12.10/95,14/95; Flow rate: 18 ml/min Flow.; Solubility: Water+THF+MeCN

### EXAMPLE 2: SYNTHESIS OF BRAF DEGRADERS

Synthesis BI: Synthesis of (3S)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]-1-piperidyl]acetyl]-1-oxa-8-azaspiro[4.5]decane (B-1):

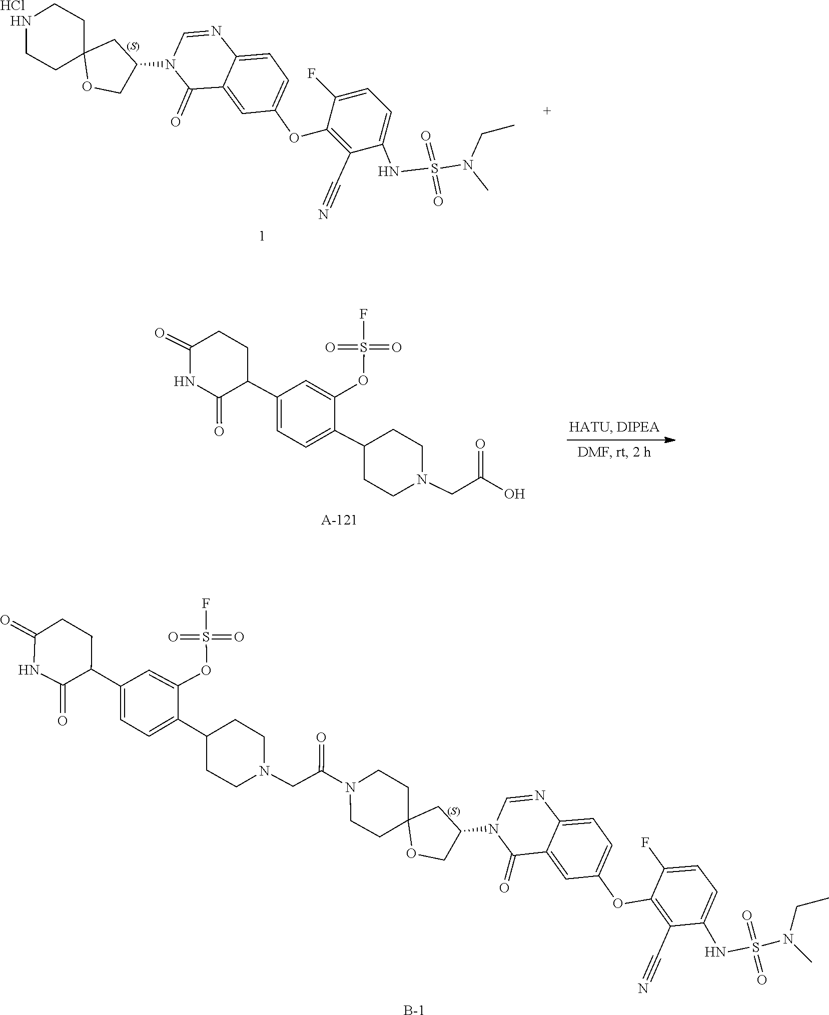

Into a 10 mL single neck round bottom flask containing a well-stirred solution of 2-[4-[4-(2,6-dioxo-3-piperidyl)-2-fluorosulfonyloxy-phenyl]-1-piperidyl]acetic acid A-121 (45 mg, 105.03 mol) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (62.29 mg, 105.03 mol, HCl salt) in dimethylformamide (1 mL) were added N,N-diisopropylethylamine (420.14 mol, 73.18 L) and HATU (79.87 mg, 210.07 mol) at 0 C. The reaction mixture was stirred at room temperature for 2 hours and the progress of the reaction was monitored by LCMS. After completion of the reaction, the reaction mixture was concentrated under reduced pressure to 15 get a crude residue, which was purified using RediSep Rf Gold® 60 g column and [Mobile phase A: 10 mM NH4HCO3 in mill-Q water; Mobile phase B: CH3CN], while the desire product was eluting at 35-40% of the mobile phase. The fractions were collected and lyophilized to afford (3S)-3-[6-[2-cyano-3- [[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]-1-piperidyl]acetyl]-1-oxa-8-azaspiro[4.5]decane (B-1) (37 mg, 36% yield) as an off-white solid.

UPLC-MS (ES+): m z 967.2 [M+H]+. H-NMR (400 MHz, DMSO-d6): δ 10.90 (s, 1H), 8.35 (s, 1H), 7.78 (d, J=8.80 Hz, 1H), 7.66 (dd, J=2.80, 9.00 Hz, 1H), 7.63-7.35 (m, 7H), 5.35-5.25 (m, 1H), 4.17-4.13 (m, 2H), 4.01-3.98 (m, 1H), 3.78-3.72 (m, 1H), 3.57-3.51 (m, 3H), 3.42-3.38 (m, 2H), 3.21-2.85 (m, 5H), 2.69-2.65 (m, 5H), 2.56-2.53 (m, 2H), 2.42-2.39 (m, 1H), 2.28-2.23 (m, 1H), 2.13-2.01 (m, 2H), 1.88-1.52 (m, 8H), 1.04 (t, J=7.20 Hz, 3H).

Synthesis B2: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]-1-piperidyl]acetyl]-1-oxa-8-azaspiro[4.5]decane (B-2):

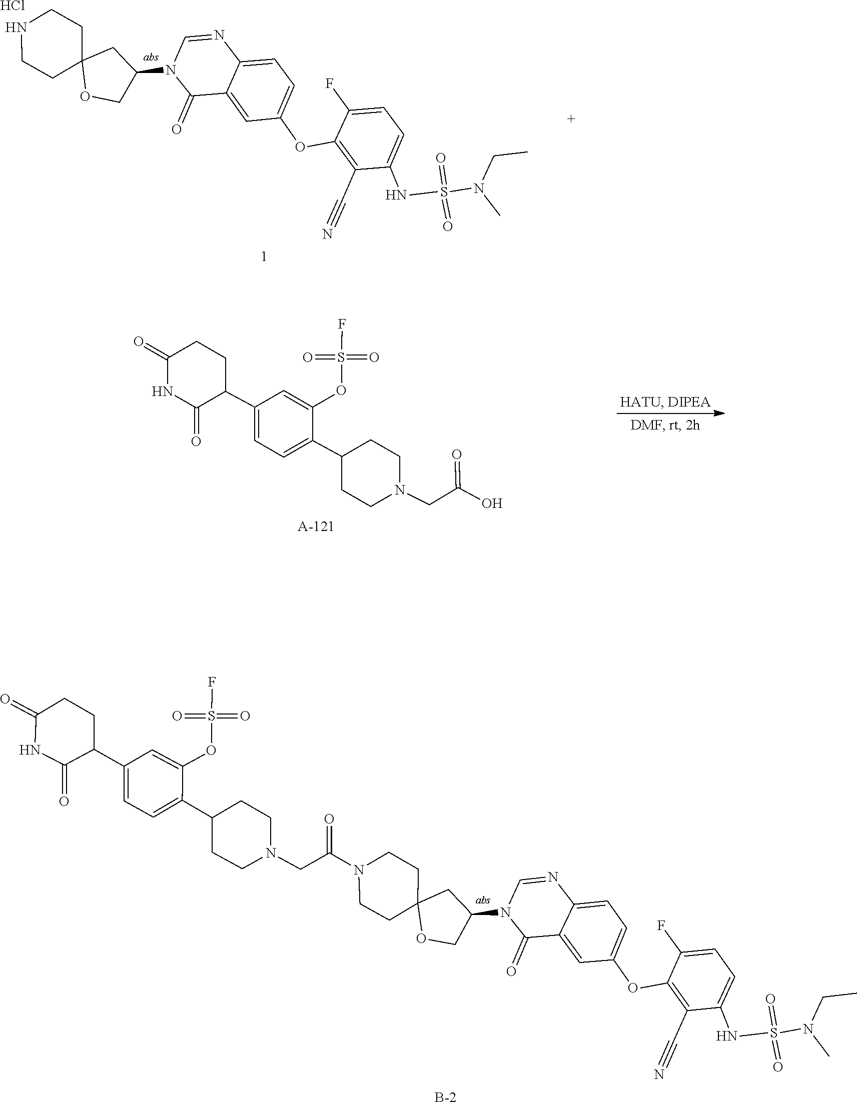

Into a 10 mL single neck round bottom flask containing a well-stirred solution of 2-[4-[4-(2,6-dioxo-3-piperidyl)-2-fluorosulfonyloxy-phenyl]-1-piperidyl]acetic acid A-121 (48 mg, 85.15 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (50.50 mg, 85.15 mol, HCl salt) in dimethylformamide (1.00 mL) were added N,N-diisopropylethylamine (340.59 mol, 59.32 L) and HATU (64.75 mg, 170.30 mol) at 0° C., stirred at room temperature for 2 hours, The reaction was monitored by UPLC-MS. After completion of the reaction, reaction mixture was concentrated under reduced pressure to get crude which was purified by using RediSep Rf Gold® 60 g column and [Mobile phase A: 10 mM NH4HCO3 in milli-Q water; Mobile phase 20 B: CH3CN], while the desire product was eluting at²5-³0% of the mobile phase, the fractions were collected and lyophilized to get (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]-1-piperidyl]acetyl]-1-oxa-8-azaspiro[4.5]decane (B-2) (30 mg, 36% yield) as an off-white solid. UPLC-MS (ES*): m z 967.6 [M+H]+. ¹H NMR (400 MHz, DMSO-d6): δ 10.90 (s, 1H), 9.89 (bs, 1H), 8.35 (s, 1H), 7.78 (d, J=8.80 Hz, 1H), 7.66 (dd, J=3.20, 9.00 Hz, 1H), 7.62-7.36 (m, 6H), 5.34-5.27 (m, 1H), 4.21-4.13 (m, 2H), 4.02-3.98 (m, 1H), 3.78-3.67 (m, 2H), 3.58-3.45 (m, 3H), 3.23-3.15 (m, 2H), 3.07 (q, J=6.80 Hz, 3H), 2.92-2.85 (m, 1H), 2.71-2.56 (m, 5H), 2.42-2.21 (m, 4H), 2.13-2.01 (m, 2H), 1.94-1.52 (m, 8H), 1.04 (t, J=7.20 Hz, 3H).

Synthesis B3: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]piperazin-1-yl]acetyl]-1-oxa-8-azaspiro[4.5]decane (B-3):

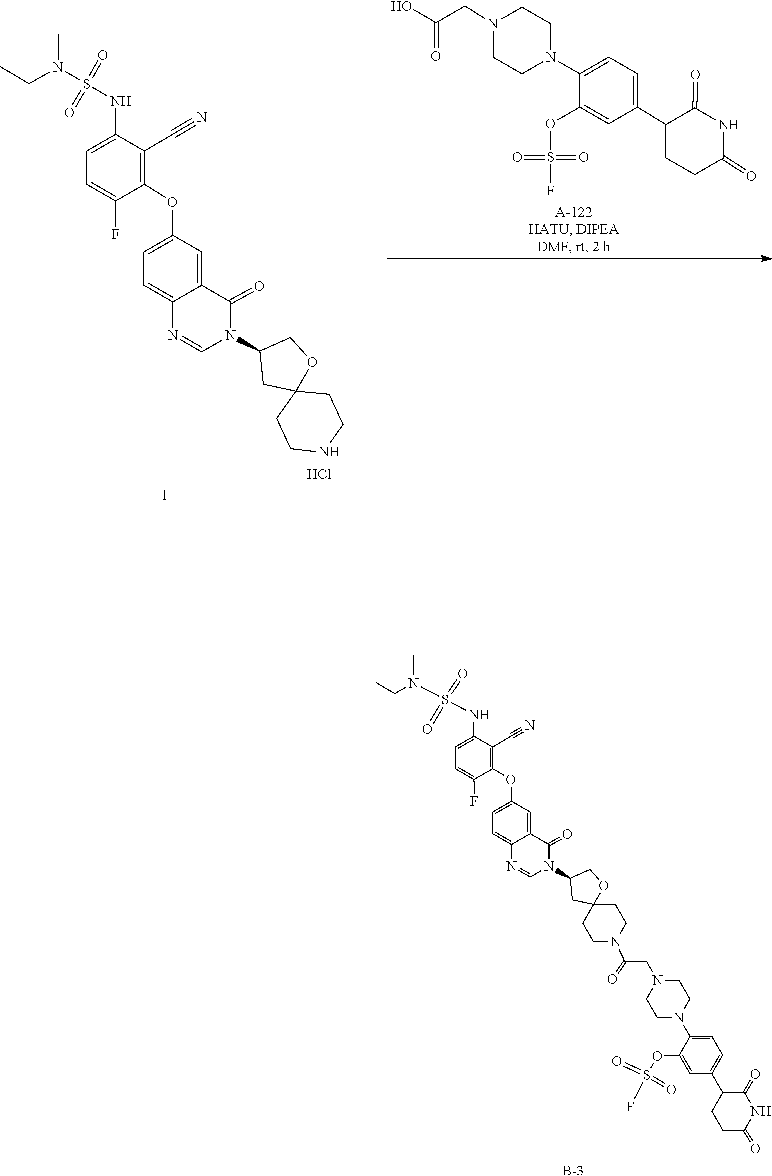

Into a 10 mL single-neck round-bottom flask containing a well-stirred solution of 2-[4-[4-(2,6-dioxo-3-piperidyl)-2-fluorosulfonyloxy-phenyl]piperazin-1-yl]acetic acid A-122 (46 mg, 107.12 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (63.53 mg, 107.12 mol, HCl salt) in dimethylformamide (1.2 mL) were added N,N-diisopropylethylamine (428.49 mol, 74.63 L) and HATU (81.46 mg, 214.24 mol) at 0° C. Resulting mixture was stirred at room temperature for 2 hours. Progress of the reaction was monitored by UPLC-MS. After completion of the reaction, reaction mixture was concentrated under reduced pressure to get crude, which was purified by using RediSep Rf Gold® 60 g column and [Mobile phase A: 10 mM NH4HCO3 in milli-Q water; Mobile phase B: CH3CN]. Eluted fractions were lyophilized to get 60 mg of compound (HPLC purity, 84%). Further purified by prep-HPLC by using method: [X BRIDGE C18, (150 mm×19 mm×5 pm), Mobile phase A: 10 mM NH4HCO3 (pH:10 adjust with Ammonia) in milli-Q water, Mobile phase B: CH-3CN, Flow:15 mLUmin), eluted fractions were lyophilized to afford (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]piperazin-1-yl]acetyl]-1-oxa-8-azaspiro[4.5]decane (B-3) (32 mg, 32.57 mol, 30% yield) as an off-white solid. UPLC-MS (ESI): m z 968.1 [M+H]+H-NMR (400 MHz, DMSO-d6): δ 10.86 (s, 1H), 10.16 (bs, 1H), 8.35 (s, 1H), 7.80-7.67 (m, 3H), 7.42-7.27 (m, 5H), 5.35-5.25 (m, 1H), 4.16-4.12 (m, 2H), 3.95-3.89 (m, 1H), 3.74-3.65 (m, 1H), 3.62-3.44 (m, 2H), 3.38-3.36 (m, 2H), 3.14-2.98 (m, 7H), 2.75-2.63 (m, 7H), 2.55-2.53 (m, 2H), 2.43-2.38 (m, 1H), 2.28-2.22 (m, 1H), 2.12-1.95 (m, 2H), 1.80-1.52 (m, 4H), 1.05 (t, J=7.20 Hz, 3H.

Synthesis B4: Synthesis of (3S)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]-1-piperidyl]acetyl]-1-oxa-8-azaspiro[4.5]decane (B-4):

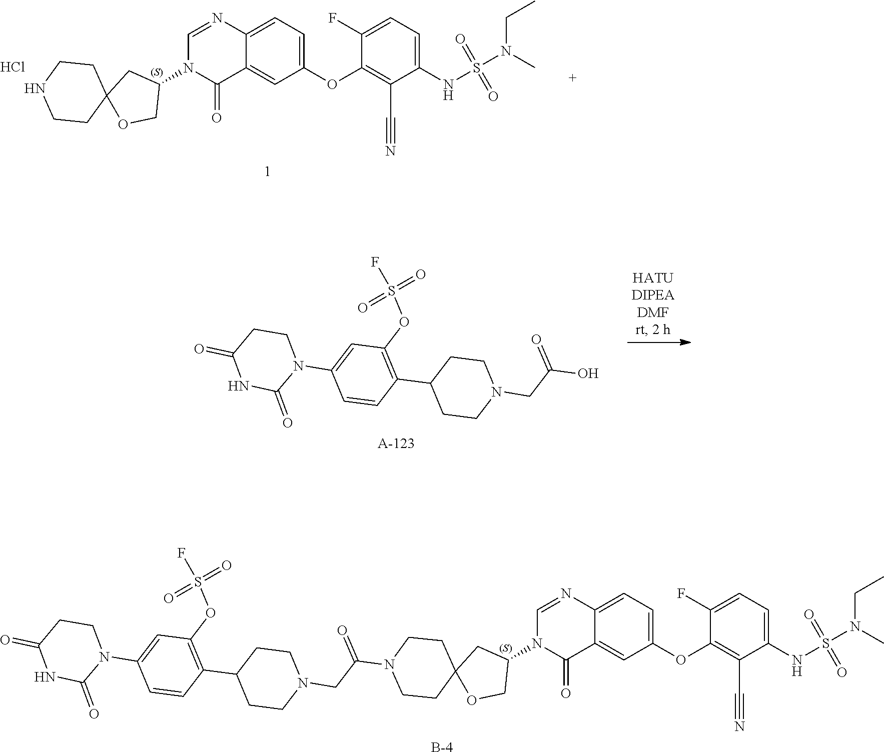

fluoro-phenoxy]-4-oxo-quinazolin-3-yl]-1-oxa-8-azaspiro[4.5]decane 1 (47.20 mg, 79.59 mol, HCl salt) in dimethylformamide (2 mL) was added N,N-diisopropylethylamine (397.93 mol, 69.31 L) at room temperature. Subsequently, HATU (60.52 mg, 159.17 mol) was added at 0° C. and the resulting reaction mixture was stirred at room temperature for 2 hours. 5 Progress of the reaction was monitored by UPLC-MS. After the completion of the reaction, the reaction mixture was concentrated under reduced pressure to get a crude residue which was purified by preparative HPLC by using a method: column: X Bridge C18 (250 mm* 19 mm*5 L) and [Mobile phase A:10 mM NH4HCO3 & NH40H (pH adjusted to 10) in milli-Q water; Mobile phase B: CH3CN]. Pure fractions were lyophilized to afford (3S)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]-1-piperidyl]acetyl]-1-oxa-8-azaspiro[4.5]decane (B-4) (35 mg, 36.05 mol, 45% yield) as an off-white solid. UPLC-MS (ES*): m z 968.3 [M+H]+. H-NMR (400 MHz, DMSO-d6): 6.51 (s, 1H), 9.91 (bs, 1H), 8.35 (s, 1H), 7.78 (d, J=8.80 Hz, 1H), 7.68-7.52 (m, 5H), 7.38-7.36 (m, 2H), 5.36-5.28 (m, 1H), 4.21-4.09 (m, 2H), 3.84 (t, J=6.40 Hz, 2H), 3.78-3.42 (m, 4H), 3.42-3.36 (m, 2H), 3.28-3.13 (m, 2H), 3.10-3.02 (m, 2H), 2.99-2.85 (m, 1H), 2.74-2.68 (m, 5H), 2.60-2.55 (m, 1H), 2.45-2.31 (m, 2H), 2.15-2.05 (m, 1H), 2.00-1.50 (m, 8H), 1.04 (t, J=7.20 Hz, 3H).

Synthesis B5: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]-1-piperidyl]acetyl]-1-oxa-8-azaspiro[4.5]decane (B-5):

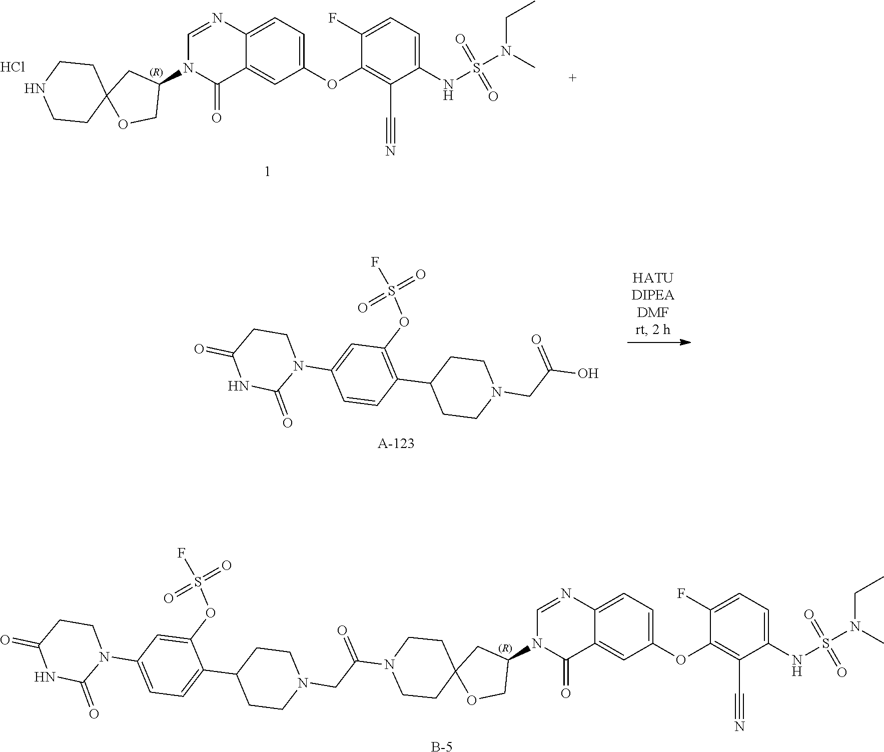

Into a 10 mL single neck round bottom flask containing a well-stirred solution of 2-[4-[4-(2,4-dioxohexahydropyrimidin-1-yl)-2-fluorosulfonyloxy-phenyl]-1-piperidyl]acetic acid A-123 (32 mg, 51.42 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (30.49 mg, 51.42 mol, HCl salt) in dimethylformamide (1.2 mL) were added N,N-diisopropylethylamine (205.67 mol, 35.82 L) and HATU (39.10 mg, 102.84 mol) at 0° C., stirred at room 5 temperature for 2 hours. The reaction was monitored by UPLC-MS. After completion of the reaction, reaction mixture was concentrated under reduced pressure to get crude which was purified by using RediSep Rf Gold® 60 g column and [Mobile phase A: 10 Mm NH4HCO3 in milli-Q water; Mobile phase B: CH3CN], while the desire product was eluting at 25-30% of the mobile phase, the fractions were lyophilized to get (3R)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]-1-piperidyl]acetyl]-1-oxa-8-azaspiro[4.5]decane (B-5) (21 mg, 41% yield) as an off-white solid. UPLC-MS (ES*): m z 968.6 [M+H]+. ¹H NMR (400 MHz, DMSO-d6): δ 10.51 (s, 1H), 9.93 (bs, 1H), 8.35 (s, 1H), 7.78 (d, J=8.80 Hz, 1H), 7.66 (dd, J=2.80, 9.00 Hz, 1H), 7.59-7.49 (m, 4H), 7.39-7.33 (m, 2H), 5.34-5.26 (m, 1H), 4.17-4.13 (m, 2H), 3.84 (t, J=6.40 Hz, 2H), 3.76-3.71 (m, 2H), 3.58-3.42 (m, 5H), 3.21-3.12 (m, 2H), 3.06 (q, J=7.20 Hz, 2H), 3.94-3.83 (m, 1H), 2.73 (t, J=6.40 Hz, 2H), 2.66 (s, 3H), 2.42-2.34 (m, 1H), 2.15-2.05 (m, 1H), 1.92-1.54 (m, 8H), 1.04 (t, J=7.20 Hz, 3H). Note: One proton is obscured by the moisture peak.

Synthesis B6: Synthesis of (3S)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]piperazin-1-yl]acetyl]-1-oxa-8-azaspiro[4.5]decane (B-6):

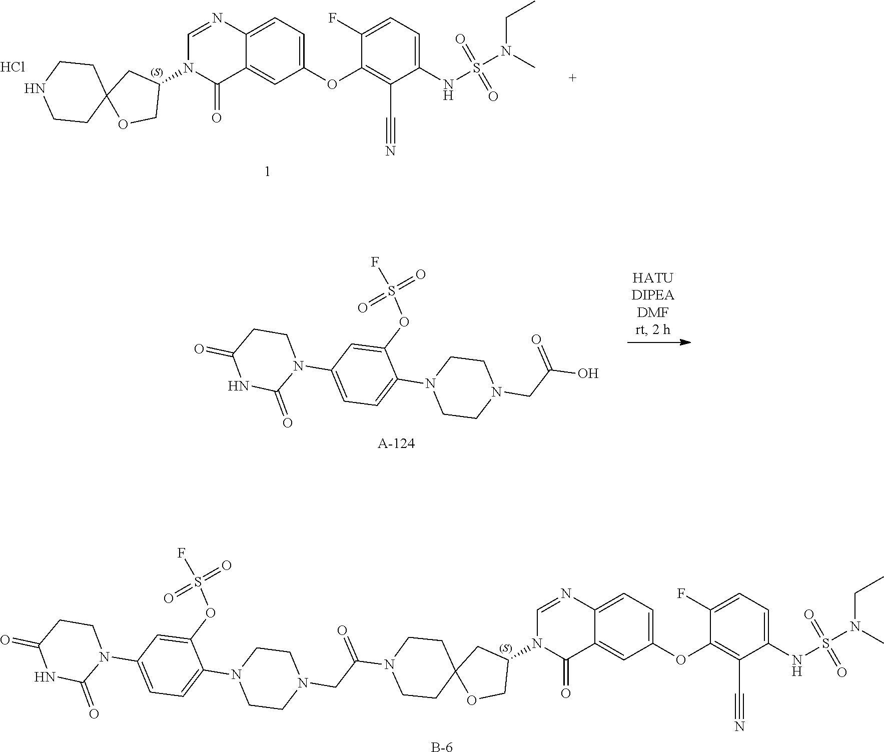

1-oxa-8-azaspiro[4.5]decane 1 (60 mg, 91.06 mol, HCl salt) and 2-[4-[4-(2,4-dioxohexahydropyrimidin-1-yl)-2-fluorosulfonyloxy-phenyl]piperazin-1-yl]acetic acid A-124 (48 mg, 97.02 mol) in anhydrous dimethylformamide (419.76 L) were added N,N-diisopropylethylamine (485.12 mol, 84.50 pL) and HATU (44.27 mg, 116.43 μmol) at 5 ambient temperature. The resulting mixture was stirred at ambient temperature for 2 hours.

After the completion of the reaction, the volatiles were removed under reduced pressure to get a crude residue, which was purified by the reverse phase Column: Redisep Rf Gold ® reversed-phase C18-teledyne ISCO, 120 g, [Mobile phase A: 10 mM NH4HCO3 in mill-Q water; Mobile phase B: CH3CN]. The pure fractions were lyophilized to afford (3S)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]piperazin-1-yl]acetyl]-1-oxa-8-azaspiro[4.5]decane (B-6) (35 mg, 37% yield) as an off-white solid. UPLC-MS (ES*): m z 969.1 [M+H]+. ¹H NMR (400 MHz, DMSO-d6): δ 10.44 (s, 1H), 8.38 (s, 1H), 7.80-7.67 (m, 3H), 7.56 (s, 1H), 7.46-7.28 (m, 4H), 5.42-5.35 (m, 1H), 4.17-4.09 (m, 2H), 3.78 (t, J=6.40 Hz, 2H), 3.73-3.56 (m, 4H), 3.33-3.26 (m, 2H), 3.12 (q, J=7.20 Hz, 3H), 3.02 (s, 4H), 2.74-2.68 (m, 8H), 2.42-2.34 (m, 1H), 2.11-2.04 (m, 1H), 1.79-1.53 (m, 4H), 1.04 (t, J=12.00 Hz, 3H).

Synthesis B7: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]piperazin-1-yl]acetyl]-1-oxa-8-azaspiro[4.5]decane (B-7):

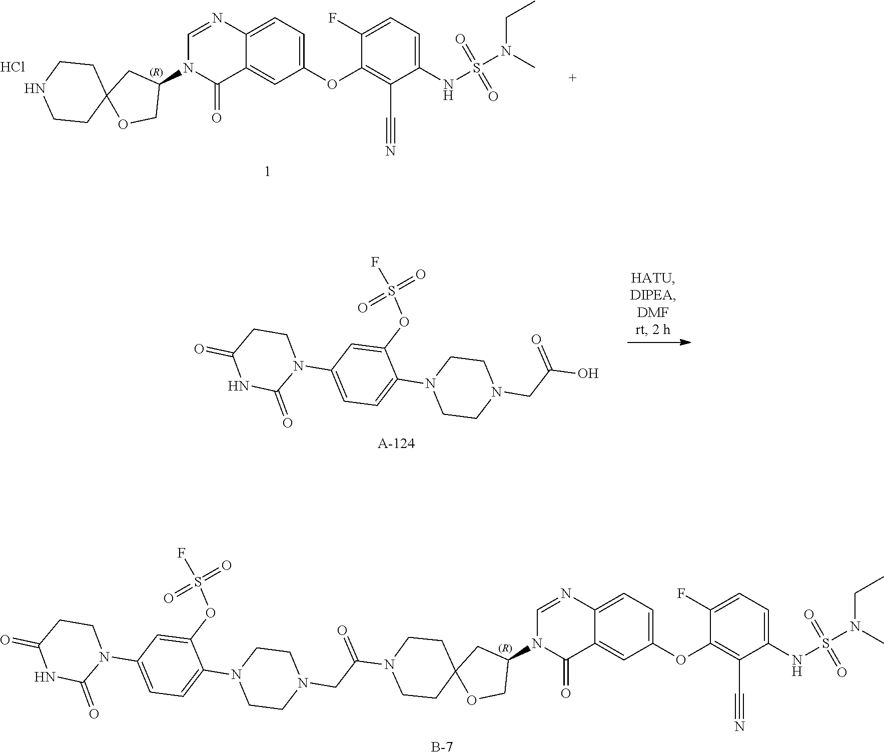

Into a 25 mL single-neck round-bottom flask containing a well-stirred solution of 2-[4-25 [4-(2,4-dioxohexahydropyrimidin-1-yl)-2-fluorosulfonyloxy-phenyl]piperazin-1-yl]acetic acid A-124 (40 mg, 69.70 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (41.34 mg, 69.70 mol, HCl salt) in anhydrous dimethylformamide (496.60 L) was added N,N-diisopropylethylamine (348.51 mol, 60.70 pL) followed by and HATU (31.80 mg, 83.64 5 μmol) at room temperature and stirred for 2 hours. Progress of the reaction was monitored by UPLC-MS. After completion of the reaction, reaction mixture was concentrated under reduced pressure to get a crude residue, which was purified by reverse phase [Column: Redisep Rf Gold® reversed-phase C18-teledyne ISCO,120 g, Mobile phase A:10 mM NH4HCO3 in milli-Q water; Mobile phase B: CH3CN]. The collected fractions were lyophilized to afford (3R)-3-[6-[2-cyano-3-[[ethyl(methyl)sulfamoyl]amino]-6-fluoro-phenoxy]-4-oxo-quinazolin-3-yl]-8-[2-[4-[4-(2,4-dioxohexahydropyrimidin-1-yl)-2-fluorosulfonyloxy-phenyl]piperazin-1-yl]acetyl]-1-oxa-8-azaspiro[4.5]decane (B-7) (16 mg, 15.75 μmol, 23% yield) as an off-white solid. UPLC-MS (ES+): m z 969.2 [M+H]+. ¹H NMR (400 MHz, DMSO-d6): δ 10.44 (s, 1H), 8.38 (s, 1H), 8.22 (bs, 1H), 6 7.75 (d, J=8.80 Hz, 1H), 7.62 (dd, J=2.80, 8.80 Hz, 1H), 7.56 (s, 1H), 7.46-7.28 (m, 5H), 5.42-5.15 (m, 1H), 4.19-4.09 (m, 2H), 3.78 (t, J=6.40 Hz, 2H), 3.72-3.51 (m, 3H), 3.30-3.20 (m, 3H), 3.19-3.09 (m, 2H), 3.05-2.91 (m, 6H), 2.75-2.69 (m, 2H), 2.62-2.55 (m, 6H), 2.43-2.37 (m, 1H), 2.17-2.00 (m, 1H), 1.82-1.65 (m, 3H), 1.02 (t, J=7.20 Hz, 3H).

Synthesis B8: Synthesis of (3S)-3-[6-[2-cyano-3-[[ethyl(methyl)sulfamoyl]amino]-6-fluoro-phenoxy]-4-oxo-quinazolin-3-yl]-8-[2-[4-[4-[(2,6-dioxo-3-piperidyl)oxy]-2-fluorosulfonyloxy-phenyl]-1-piperidyl]acetyl]-1-oxa-8-azaspiro[4.5]decane (B-8):

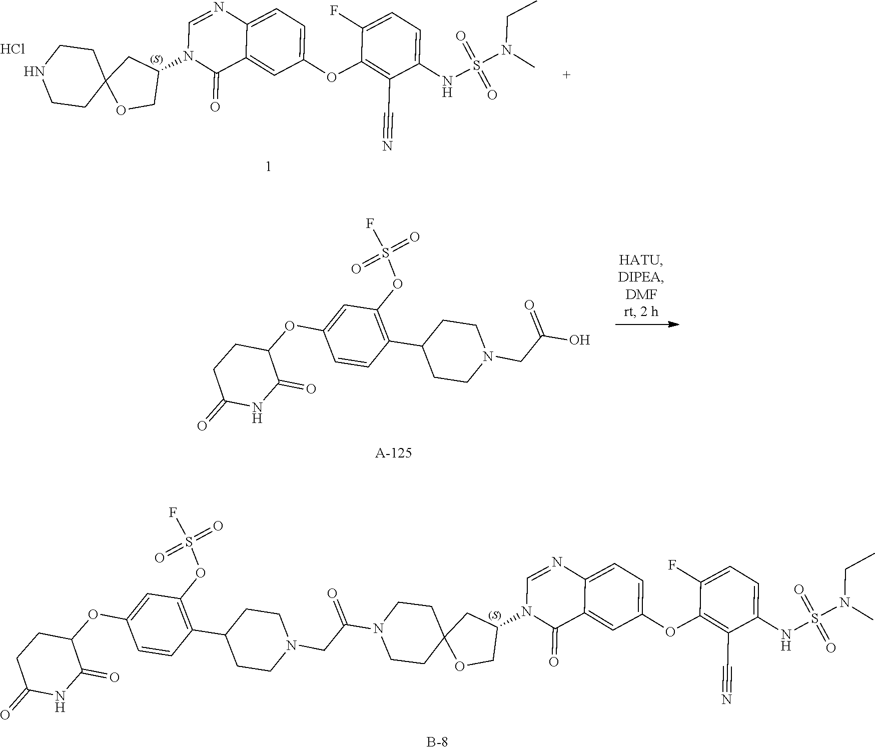

Into a 25 mL single-neck round-bottom flask containing a well-stirred solution of 2-[4-[4-[(2,6-dioxo-3-piperidyl)oxy]-2-fluorosulfonyloxy-phenyl]-1-piperidyl]acetic acid A-125 (55 mg, 86.76 mol) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (51.46 mg, 86.76 mol, HCl salt) in dimethylformamide (1 mL) were added N,N-diisopropylethylamine (86.76 mol, 15.11 L) and HATU (49.49 mg, 130.15 mol) at 0° C. Resulting mixture was stirred at room temperature for 1 hour. Progress of the reaction monitored by UPLC-MS. After the completion of the reaction, the reaction mixture was concentrated under reduced pressure to get a crude residue, which was purified by preparative HPLC (Column: X Bridge C18 (150 mm*19 mm*5 pL), Mobile phase A: 10 mM NH4HCO3 (pH was adjusted to 10 with NH40H) in milli-Q water; Mobile phase B: CH3CN, flow rate:14 mL/min). Pure fractions were combined and lyophilized to afford (3S)-3-[6-[2-cyano-3- [[ethyl(methyl)sulfamoyl]amino]-6-fluoro-phenoxy]-4-oxo-quinazolin-3-yl]-8-[2-[4-[4-[(2,6-dioxo-3-piperidyl)oxy]-2-fluorosulfonyloxy-phenyl]-1-piperidyl]acetyl]-1-oxa-8-azaspiro[4.5]decane (B-8) (34.50 mg, 40% yield) as an off-white solid. UPLC-MS (ES+): m z 983.2 [M+H]+. H-NMR (400 MHz, DMSO-d6): δ 10.99 (s, 1H), 9.88 (s, 1H), 8.35 (s, 1H), 7.78 (d, J=9.20 Hz, 1H), 7.67-7.36 (m, 5H), 7.24-7.18 (m, 2H), 5.34-5.30 (m, 2H), 4.17-4.13 (m, 2H), 3.67-3.78 (m, 1H), 3.42-3.63 (m, 3H), 3.37-3.41 (m, 1H), 2.98-3.27 (m, 4H), 2.85-2.67 (m, 7H), 2.53-2.62 (m, 2H), 2.44-2.37 (m, 1H), 2.22-2.12 (m, 3H), 1.92-1.55 (m, 8H), 1.04 (t, J=7.20 Hz, 3H).

Synthesis B9: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-[(2,6-dioxo-3-piperidyl)oxy]-2-fluorosulfonyloxy-phenyl]-1-piperidyl]acetyl]-1-oxa-8-azaspiro[4.5]decane (B-9):

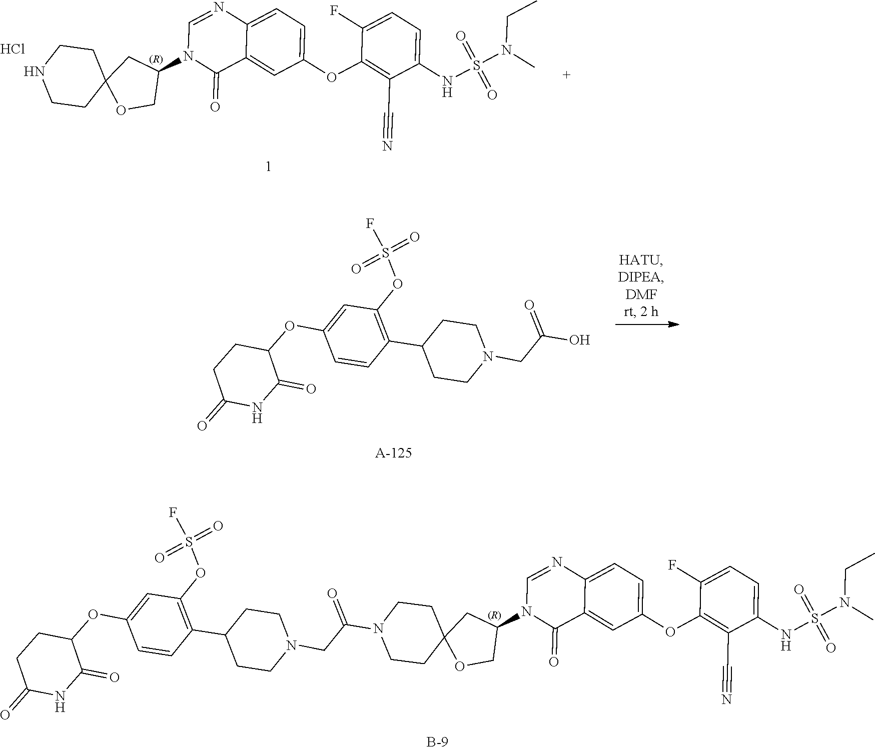

Into a 25 mL single-neck round-bottom flask containing a well-stirred solution of 2-[4-[4-[(2,6-dioxo-3-piperidyl)oxy]-2-fluorosulfonyloxy-phenyl]-1-piperidyl]acetic acid A-125 (40 mg, 82.80 mol) in anhydrous dimethylformamide (1 mL) was added N,N-diisopropylethylamine (331.21 mol, 57.69 pL) followed by (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (46.09 mg, 77.71 μmol, HCl salt) and HATU (37.78 mg, 99.36 mol) at room temperature and stirred for 2 hours. Progress of the reaction was monitored by UPLC-MS. After the completion of the reaction, the reaction mixture was concentrated under reduced pressure to get a crude residue, which was purified by reverse phase [Column: Redisep Rf Gold® reversed-phase C18-teledyne ISCO,120 g, Mobile phase A:10 mM NH4HCO3 in milli-Q water; Mobile phase B: CH3CN]. The pure fractions were lyophilized to afford (3R)-3-[6-[2-cyano-3-[[ethyl(methyl)sulfamoyl]amino]-6-fluoro-phenoxy]-4-oxo-quinazolin-3-yl]-8-[2-[4-[4-[(2,6-dioxo-3-piperidyl)oxy]-2-fluorosulfonyloxy-phenyl]-1-piperidyl]acetyl]-1-oxa-8-azaspiro[4.5]decane (B-9) (13 mg, 12.83 mol, 16% yield) as an off-white solid. UPLC-MS (ES*): m z 983.6 [M+H]+. ¹H-NMR (400 MHz, DMSO-d6): δ 10.99 (s, 1H), 8.35 (s, 1H), 7.78 (d, J=9.20 Hz, 1H), 7.63 (dd, J=2.80, 9.20 Hz, 1H), 7.52-7.11 (m, 6H), 5.37-5.21 (m, 2H), 4.21-4.11 (m, 2H), 3.68-3.67 (m, 2H), 3.35-3.32 (m, 2H), 3.04-2.94 (m, 4H), 2.81-2.65 (m, 3H), 2.57 (s, 3H), 2.42-2.35 (m, 2H), 2.25-1.99 (m, 4H), 1.83-1.49 (m, 8H), 1.04 (t, J=7.20 Hz, 3H).

Synthesis B10: Synthesis of [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,4-dioxohexahydropyrimidin-1-yl)phenyl]methanesulfonate (B-10):

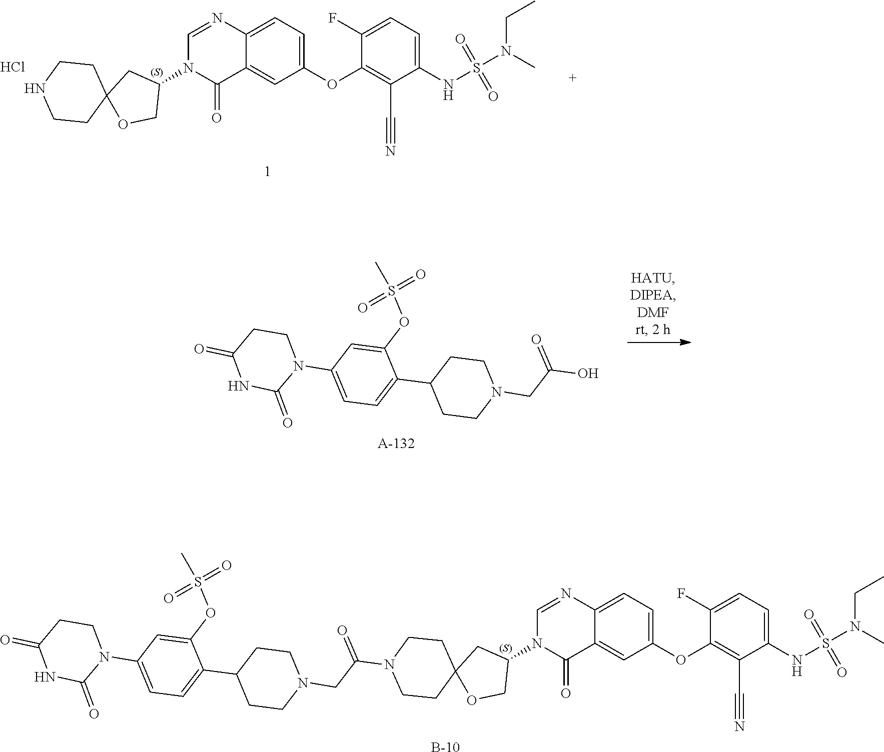

Into a 25 mL single-neck round-bottom flask containing a well-stirred solution of 2-[4-[4-(2,4-dioxohexahydropyrimidin-1-yl)-2-methylsulfonyloxy-phenyl]-1-piperidyl]acetic acid A-132 (60 mg, 90.00 mol) in dimethylformamide (1.00 mL) were added N,N-diisopropylethylamine (360.01 mol, 62.71 L) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (53.38 mg, 90.00 mol, HCl salt) at room temperature.

Subsequently, HATU (51.33 mg, 135.00 mol) was added at the same temperature and the resulting reaction mixture was stirred at room temperature for 2 hours. Progress of the reaction was monitored by UPLC-MS. After completion of the reaction, the reaction mixture was 10 concentrated directly under reduced pressure to get a crude residue which was purified by preparative HPLC by using method [Column: X Bridge C18 (150 mm*19 mm*5pL) and Mobile phase A: 10 mm NH4HCO3 & NH40H (pH-10) in milli-Q water; Mobile phase B: CH3CN]. The pure fractions were lyophilized to afford [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,4-dioxohexahydropyrimidin-1-yl)phenyl]methanesulfonate (B-10) (25 mg, 29% yield) as a white solid. UPLC-MS (ES+): m z 964.3 [M+H]+. H-NMR (400 MHz, DMSO-d6): δ 10.46 (s, 1H), 9.35 (bs, 1H), 8.35 (s, 1H), 7.78 (d, J=8.80 Hz, 1H), 7.68-7.33 (m, 7H), 5.31-5.35 (m, 1H), 4.20-4.12 (m, 2H), 3.84-3.72 (m, 4H), 3.53-3.51 (m, 5H), 3.44-3.33 (m, 2H), 3.09-3.04 (m, 6H), 2.74-2.50 (m, 4H), 2.44-2.40 (m, 2H), 2.25-1.69 (m, 10H), 1.04 (t, J=7.20 Hz, 3H).

Synthesis B11: Synthesis of [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,4-dioxohexahydropyrimidin-1-yl)phenyl]methanesulfonate (B-11):

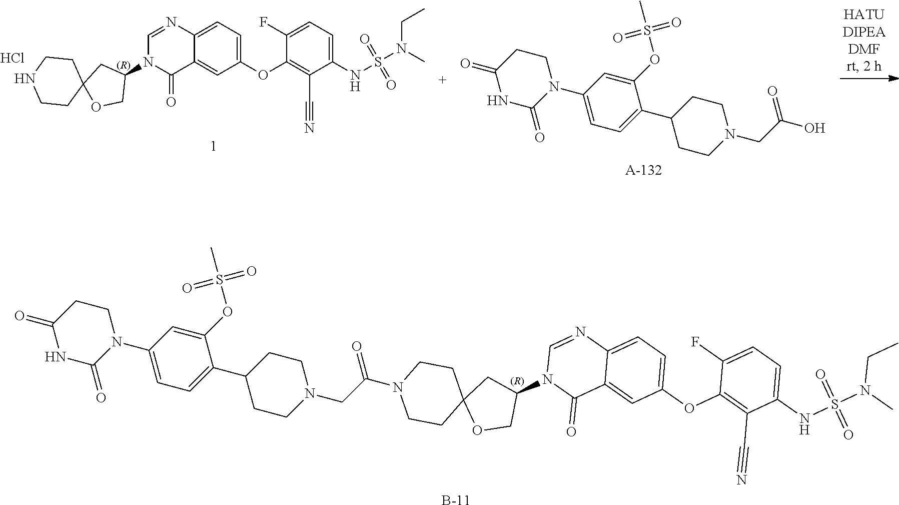

Into a 25 mL single-neck round-bottom flask containing a well-stirred solution of 2-[4-[4-(2,4-dioxohexahydropyrimidin-1-yl)-2-methylsulfonyloxy-phenyl]-1-piperidyl]acetic acid A-132 (30 mg, 21.58 mol) in dimethylformamide (1 mL) were added N,N-diisopropylethylamine (86.31 mol, 15.03 L) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (12.80 mg, 21.58 mol, HCl salt) at room temperature. Subsequently, HATU (12.31 mg, 32.37 mol) was added at the same temperature and the resulting reaction mixture was stirred at room temperature for 2 hours. Progress of the reaction was monitored 10 by UPLC-MS. After the completion of the reaction, the reaction mixture was concentrated under reduced pressure to get a crude residue, which was purified by preparative HPLC by using method [Column: X Bridge C18 (150 mm*19 mm*5 pL) and Mobile phase A:10 Mm NH4HCO3 & NH40H (pH-10) in milli-Q water; Mobile phase B: CH3CN]. The pure fractions were lyophilized to afford [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,4-dioxohexahydropyrimidin-1-yl)phenyl]methanesulfonate (B-11) (5 mg, 23% yield) as a white solid. UPLC-MS (ES+): m z 964.6 [M+H]+. H-NMR (400 MHz, DMSO-d6): δ 10.45 (s, 1H), 9.35 (bs, 1H), 8.35 (s, 1H), 7.77 (d, J=8.80 Hz, 1H), 7.64 (dd, J=3.20, 9.00 Hz, 1H), 7.50-7.32 (m, 6H), 5.38-5.30 (m, 1H), 4.20-4.10 (m, 2H), 3.82 (t, J=5.60 Hz, 2H), 3.78-3.40 (m, 7H), 3.40-3.35 (m, 3H), 3.30-2.90 (m, 6H), 2.72 (t, J=6.40 Hz, 2H), 2.68-2.60 (m, 3H), 2.45-2.35 (m, 1H), 2.25-2.15 (m, 1H), 1.90-1.50 (m, 8H), 1.03 (t, J=7.20 Hz, 3H).

Synthesis B12: Synthesis of 2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]benzenesulfonyl fluoride (B-12):

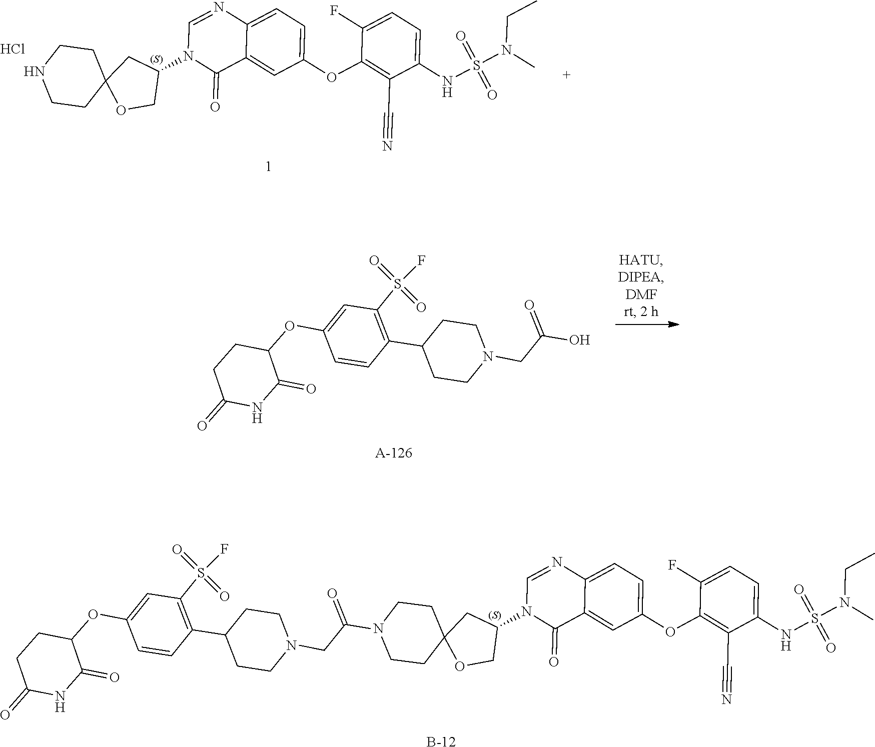

Into a 25 mL single-neck round-bottom flask containing a well-stirred solution of 2-[4-[4-[(2,6-dioxo-3-piperidyl)oxy]-2-fluorosulfonyl-phenyl]-1-piperidyl]acetic acid A-126 (45 mg, 92.43 mol) in anhydrous dimethylformamide (1.5 mL) was added N,N-diisopropylethylamine (462.15 mol, 80.50 L), followed by (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (54.82 mg, 92.43 mol, HCl salt) and HATU (42.17 mg, 110.92 mol) at room temperature and stirred for 2 hours. Progress of the reaction was monitored by UPLC-MS. After the completion of the reaction, the reaction mixture was concentrated under reduced pressure to get a crude residue, which was purified by preparatory HPLC [Column: X BRIDGE C18 (250 mm*21.0 mm*5 pL), Mobile phase A:10 mM NH4HCO3 (Ph:10 adjusted with ammonia ) in milli-Q water; Mobile phase B: CH3CN]. The pure fractions were lyophilized to afford 2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]benzenesulfonyl fluoride (B-12) (12 mg, 13% yield) as a yellow solid. UPLC-MS (ES+): m z 967.1 [M+H]+. ¹H NMR (400 MHz, DMSO-d6): δ 11.01 (s, 1H), 8.35 (s, 1H), 7.78 (d, J=8.80 Hz, 1H), 7.66 (dd, J=2.00, 8.60 Hz, 2H), 7.63-7.51 (m, 2H), 7.36 (d, J=3.20 Hz, 2H), 5.49-5.36 (m, 1H), 5.35-5.21 (m, 1H), 4.15-4.08 (m, 3H), 3.61-3.65 (m, 2H), 3.15-2.95 (m, 4H), 2.60-2.69 (m, 4H), 2.48-2.35 (m, 2H), 2.32-2.05 (m, 4H), 2.03-1.49 (m, 9H), 1.04 (t, J=6.80 Hz, 3H). Note: Six protons are obscured by the moisture.

Synthesis B13: Synthesis of 2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]benzenesulfonyl fluoride (B-13):

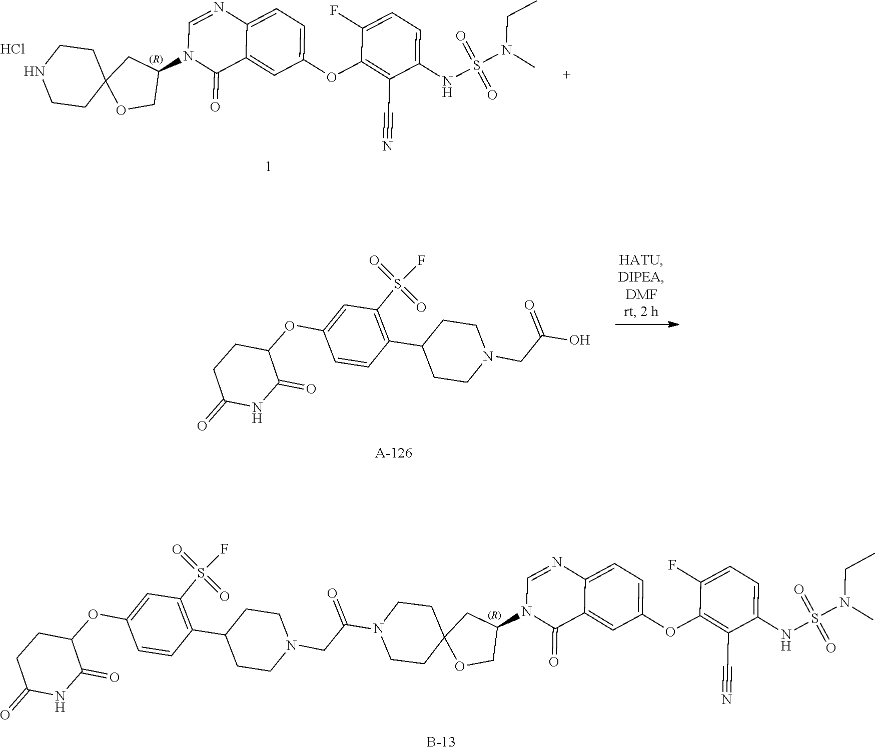

Into a 25 mL single-neck round-bottom flask containing a well-stirred solution of 2-[4-[4-[(2,6-dioxo-3-piperidyl)oxy]-2-fluorosulfonyl-phenyl]-1-piperidyl]acetic acid A-126 (40 mg, 71.89 mol) in anhydrous dimethylformamide (1.5 mL) was added N,N-diisopropylethylamine (359.45 mol, 62.61 L) followed by (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (42.64 mg, 71.89 mol, HCl salt) and HATU (32.80 mg, 86.27 mol) at room temperature and stirred for 2 hours. Progress of the reaction was monitored by UPLC-MS. After completion of the reaction, reaction mixture was concentrated under reduced 15 pressure to get a crude residue, which was purified by reverse phase purification [Column: Redisep RfGold® reversed-phase C18-teledyne ISCO, 120 g), Mobile phase A: 10 Mm NH4HCO3 in milli-Q water; Mobile phase B: CH3CN]. The fractions were lyophilized to afford 2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]benzenesulfonyl fluoride (B-13) (16 mg, 22% yield) as an off-white solid.

UPLC-MS (ES+): m z 967.6 [M+H]+. H-NMR (400 MHz, DMSO-d6): δ 11.01 (s, 1H), 8.35 (s, 1H), 7.80-7.69 (m, 2H), 7.68-7.58 (m, 2H), 7.56-7.47 (m, 1H), 7.46-7.32 (m, 2H), 7.27 (dd, J=4.00, 9.40 Hz, 1H), 5.49-5.39 (m, 1H), 5.38-5.28 (m, 1H), 4.21-4.09 (m, 2H), 3.71-3.60 (m, 3H), 3.31-3.22 (m, 2H), 3.16-3.08 (m, 2H), 3.07-2.95 (m, 4H), 2.76-2.63 (m, 2H), 2.58 (s, 3H), 2.46-2.35 (m, 1H), 2.32-2.05 (m, 5H), 1.93-1.59 (m, 8H), 1.04 (t, J=6.80 Hz, 3H) ppm.

Synthesis B14: Synthesis of 2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]benzenesulfonyl fluoride (B-14):

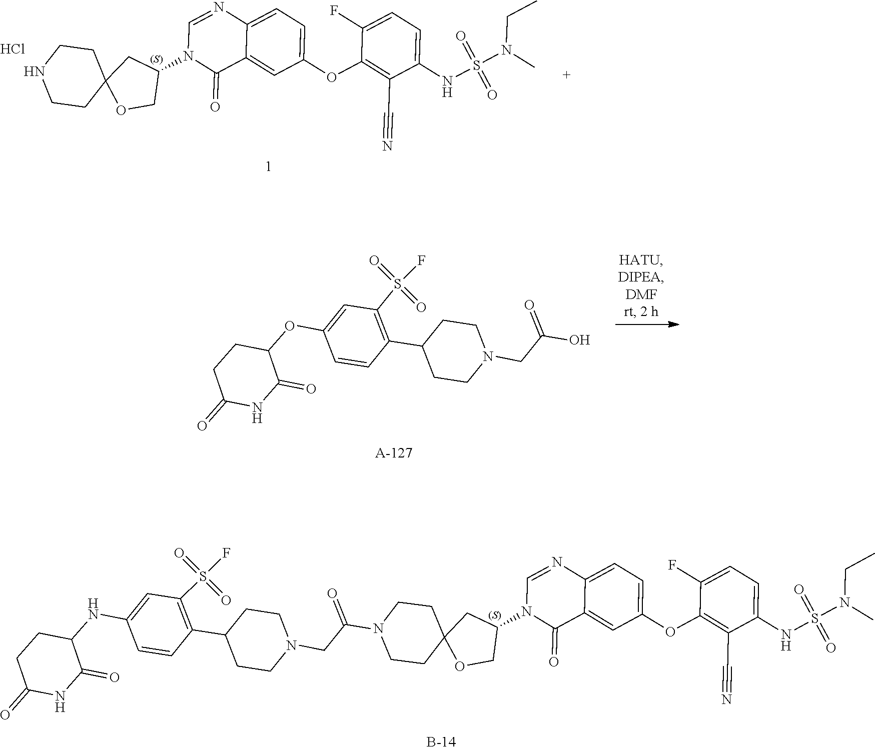

10 Into a 25 mL single-neck round-bottom flask containing a well-stirred solution of 2-[4-[4-[(2,6-dioxo-3-piperidyl)amino]-2-fluorosulfonyl-phenyl]-1-piperidyl]acetic acid A-127 (50 mg, 87.73 mol) in anhydrous dimethylformamide (1 mL) was added N,N-diisopropylethylamine (438.65 mol, 76.40 L) followed by (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (52.03 mg, 87.73 mol, HCl salt) and HATU (40.03 mg, 105.28 mol) at room temperature and stirred for 2 hours. Progress of the reaction was monitored by UPLC-MS. After completion of the reaction, reaction mixture was concentrated under reduced pressure to get a crude residue, which was purified by Prep HPLC [Column: X BRIDGE C18 (250 mm*21.0 mm*5 pL), Mobile phase A: 10 mM NH4HCO3 (pH:10 adjust with Ammonia) in milli-Q water; Mobile phase B: CH3CN]. The fractions were lyophilized to afford 2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]benzenesulfonyl fluoride (B-14) (5 mg, 6% yield) as a yellow fluffy solid. UPLC-MS (ES*): m z 966.3 [M+H]+. H-NMR (400 MHz, DMSO-d6): δ 10.83 (s, 1H), 9.85 (s, 1H), 8.34 (s, 1H), 7.78 (d, J=8.80 Hz, 1H), 7.66 (dd, J=2.80, 9.20 Hz, 1H), 7.40-7.65 (m, 2H), 7.25-7.39 (m, 3H), 7.15 (d, J=8.40 Hz, 1H), 6.67 (d, J=7.60 Hz, 1H), 5.25-5.35 (m, 1H), 4.42-4.52 (m, 1H), 4.09-4.21 (m, 2H), 3.69-3.79 (m, 2H), 3.50-3.55 (m, 3H), 3.01-3.21 (m, 5H), 2.71-2.81 (m, 1H), 2.56-2.70 (m, 5H), 2.36-2.78 (m, 2H), 2.02-2.19 (m, 2H), 1.43-2.01 (m, 9H), 1.04 (t, J=5.20 Hz, 3H). Note: One proton obscured by the moisture/solvent signal.

Synthesis B15: Synthesis of 2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]benzenesulfonyl fluoride (B-15):

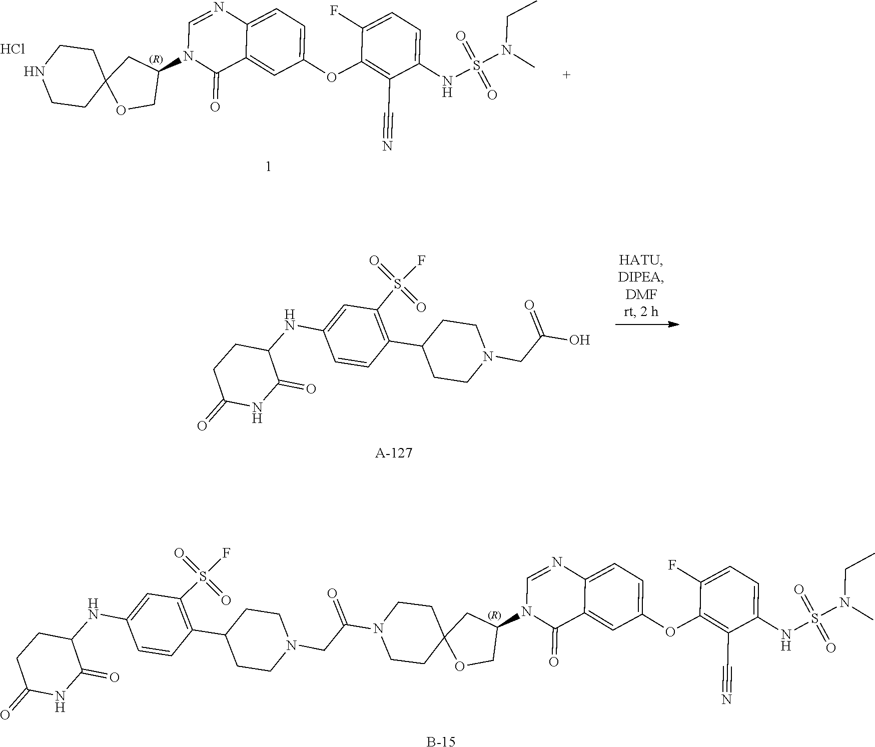

Into a 25 mL single-neck round-bottom flask containing a well-stirred solution of 2-[4-[4-[(2,6-dioxo-3-piperidyl)amino]-2-fluorosulfonyl-phenyl]-1-piperidyl]acetic acid A-127 (25 mg, 43.87 mol) in anhydrous dimethylformamide (1.04 mL) was added N,N-diisopropylethylamine (219.33 mol, 38.20 L) followed by (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (26.02 mg, 43.87 mol, HCl salt) and HATU (20.01 mg, 52.64 mol) at room temperature and stirred for 2 hours. Progress of the reaction was monitored by UPLC-MS. After completion of the reaction, reaction mixture was concentrated under reduced pressure to get a crude residue, which was purified by reverse phase [Column: Redisep Rf Gold® reversed-phase C18-teledyne ISCO,120 g, Mobile phase A:10 mM NH4HCO3 in milli-Q water; Mobile phase B: CH3CN]. The fractions were lyophilized to afford 2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]benzenesulfonyl fluoride (B-15) (12 mg, 28% yield) as an off-white solid.

UPLC-MS (ES*): m z 966.6 [M+H]+. ¹H NMR (400 MHz, DMSO-d6): δ 10.83 (s, 1H), 8.34 (s, 1H), 7.78 (d, J=8.80 Hz, 1H), 7.66 (dd, J=2.80, 9.20 Hz, 1H), 7.55-7.32 (m, 3H), 7.32-7.20 (m, 2H), 7.18-7.08 (m, 1H), 6.67 (d, J=7.60 Hz, 1H), 5.39-5.23 (m, 1H), 4.53-4.41 (m, 1H), 4.25-4.13 (m, 2H), 3.73-3.55 (m, 4H), 3.17-2.95 (m, 6H), 2.79-2.72 (m, 1H), 2.67-2.57 (m, 4H), 2.43-2.36 (m, 1H), 2.32-2.15 (m, 1H), 2.15-2.02 (m, 2H), 2.01-1.50 (m, 9H), 1.39-1.21 (m, 7H), 1.04 (t, J=5.20 Hz, 3H). Note: Four excess protons observed at (1.39-1.21) due to minor impurity in the compound.

Synthesis B16: Synthesis of [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,6-dioxo-3-piperidyl)phenyl]methanesulfonate (B-16):

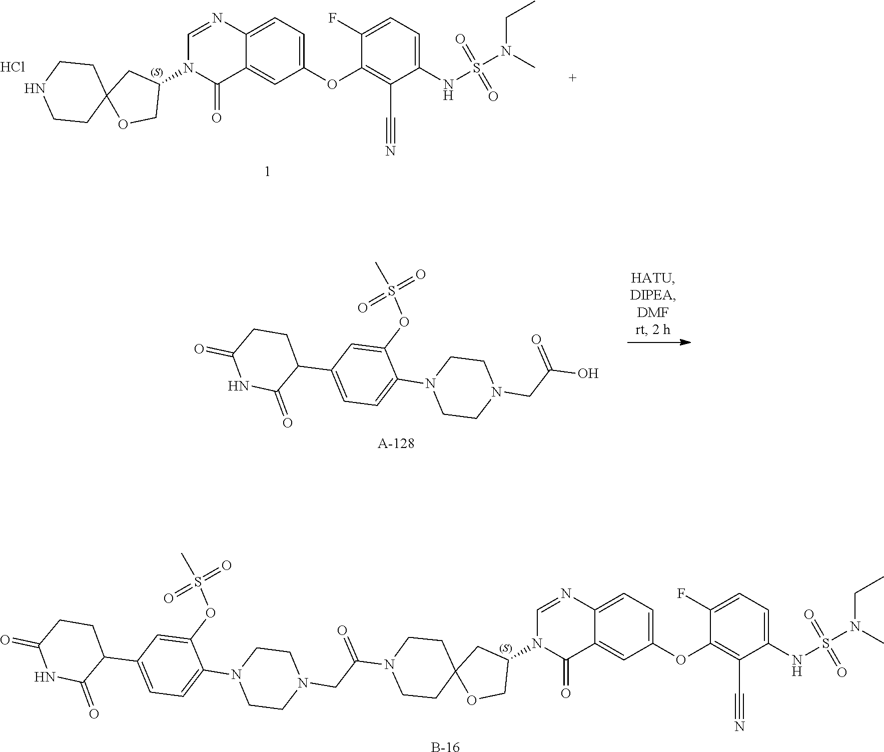

Into a 25 mL single-neck round-bottom flask containing a well-stirred solution of 2-[4-[4-(2,6-dioxo-3-piperidyl)-2-methylsulfonyloxy-phenyl]-1-piperidyl]acetic acid A-128 (60 mg, 95.26 mol) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (67.79 mg, 114.31 mol, HCl salt) in anhydrous dimethylformamide (2 mL) were added N,N-diisopropylethylamine (95.26 mol, 16.59 L) and HATU (54.33 mg, 142.89 mol) at ambient temperature. The resulting mixture was stirred at room temperature for 2 hours. Progress of the reaction was monitored by UPLC-MS. After completion of the reaction, the reaction mixture was concentrated under reduced pressure to get a crude compound, which was purified by preparative HPLC [Column: X-Bridge C18 (250 mm* 19 mm* 5 L), Mobile phase A: 10 mM NH4HCO3 in milli-Q water; Mobile phase B: CH3CN]. Pure fraction was lyophilized to afford [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-(2,6-dioxo-3-piperidyl)phenyl]methanesulfonate (B-16) (42 mg, 45% yield) as an off-white solid.

UPLC-MS (ES+): m z 963.2 [M+H]+. ¹H NMR (400 MHz, DMSO-d6): δ 10.88 (s, 1H), 9.83 (s, 1H), 8.35 (d, J=1.60 Hz, 1H), 7.77 (d, J=9.20 Hz, 1H), 7.66 (dd, J=2.80, 8.80 Hz, 1H), 7.55 (s, 1H), 7.43-7.35 (m, 3H), 7.25-7.21 (m, 2H), 5.31 (bs, 1H), 4.17-4.13 (m, 2H), 3.92 (dd, J=4.80, 12.00 Hz, 1H), 3.73 (bs, 1H), 3.52-3.48 (m, 5H), 3.40-3.34 (m, 1H), 3.23-3.15 (m, 2H), 3.08-3.03 (m, 3H), 2.69-2.65 (m, 4H), 2.56-2.50 (m, 4H), 2.34-2.33 (m, 1H), 2.25-2.21 (m, 1H), 2.11-2.04 (m, 2H), 1.92-1.68 (m, 8H), 1.04 (t, J=7.20 Hz, 3H). Note: One aliphatic proton is obscured by the moisture Synthesis B17: Synthesis of [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]phenyl]methanesulfonate (B-17):

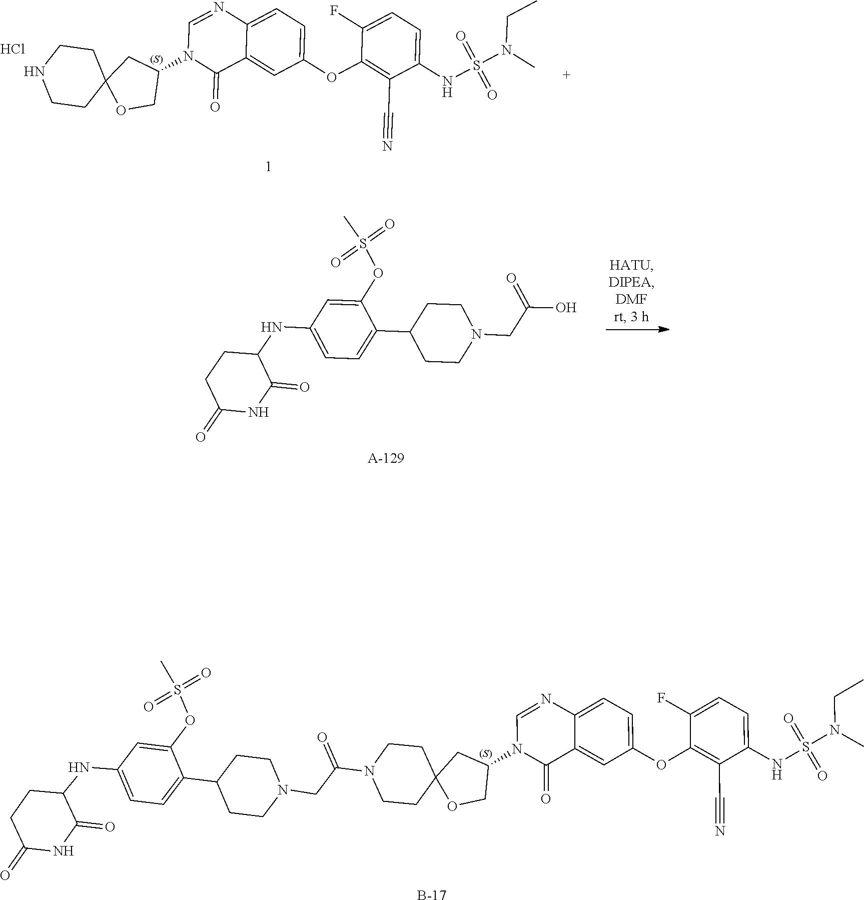

Into a 25 mL single-neck round-bottom flask containing a well stirred solution of 2-[4-[4-[(2,6-dioxo-3-piperidyl)amino]-2-methylsulfonyloxy-phenyl]-1-piperidyl]acetic acid A-129 (50 mg, 113.77 mol) in dimethylformamide (2 mL) were added N,N-diisopropylethylamine (455.08 mol, 79.27 L) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (53.98 mg, 91.02 mol, HCl salt) at room temperature. Subsequently, HATU (86.52 mg, 227.54 mol) was added at the same temperature. Resulting reaction mixture was stirred at room temperature for 3 hours. Progress of the reaction was monitored by UPLC-MS. After completion of the reaction, the reaction mixture was concentrated under reduced pressure to get crude residue which was purified by reverse phase column by using a method (Column: Redisep Rf Gold ® reversed-phase C18-teledyne ISCO, 120 g ), [Mobile phase A: 10 mM NH4HCO3 in milli-Q water; Mobile phase B: CH3CN) to afford [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]phenyl]methanesulfonate (B-17) (17 mg, 15% yield) as an off-white solid.

UPLC-MS (ES+): m z 978.2 [M+H]+. ¹H NMR (400 MHz, DMSO-d6): δ 10.81 (s, 1H), 9.83 (bs, 1H), 8.34 (d, J=1.60 Hz, 1H), 7.77 (d, J=8.80 Hz, 1H), 7.65 (dd, J=2.80, 8.80 Hz, 1H), 7.51 (bs, 1H), 7.38-7.35 (m, 2H), 7.08 (bs, 1H), 6.68-6.64 (m, 2H), 6.17 (d, J=7.20 Hz, 1H), 5.38-5.26 (m, 1H), 4.36-4.28 (m, 1H), 4.19-4.13 (m, 2H), 3.71-3.68 (m, 1H), 3.53-3.41 (m, 6H), 3.31-3.24 (m, 2H), 3.09-3.01 (m, 2H), 2.81-2.55 (m, 4H), 2.47-2.32 (m, 2H), 2.16-2.03 (m, 2H), 1.89-1.61 (m, 9H), 1.04 (t, J=7.20 Hz, 3H). Note: Five aliphatic protons are obscured by the solvent/moisture signals.

Synthesis B18: Synthesis of [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]phenyl]methanesulfonate (B-18):

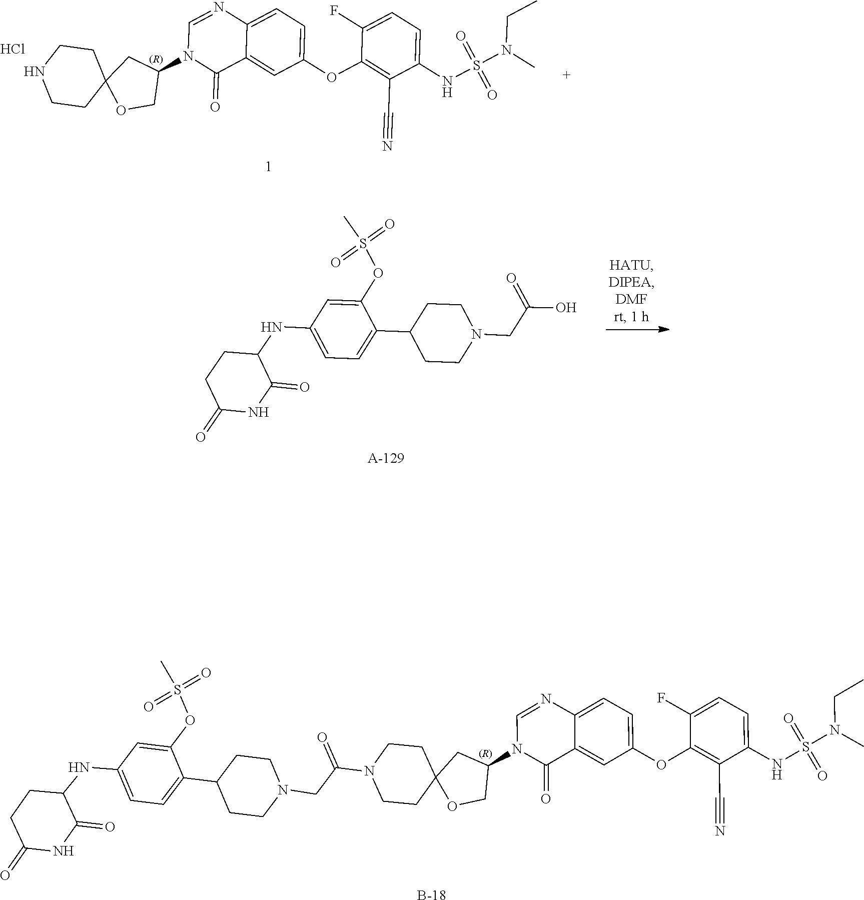

Into a 10 mL single-neck round-bottom flask containing a well stirred solution of 2-[4-[4-[(2,6-dioxo-3-piperidyl)amino]-2-methylsulfonyloxy-phenyl]-1-piperidyl]acetic acid A-129 (13 mg, 22.60 mol) in dimethylformamide (1 mL) were added N,N-diisopropylethylamine (45.20 mol, 7.87 L) and (3R)-3-[6-[2-cyano-3-[[ethyl(methyl)sulfamoyl]amino]-6-fluoro-phenoxy]-4-oxo-quinazolin-3-yl]-1-oxa-8-25 azaspiro[4.5]decane 1 (10.72 mg, 18.08 mol, HCl salt) at room temperature. Subsequently, HATU (17.19 mg, 45.20 mol) was added at same temperature. Resulting reaction mixture was stirred at room temperature for 1 hour. Progress of the reaction was monitored by UPLC-MS. After completion of the reaction, the reaction mixture was concentrated under reduced pressure to get crude residue which was purified by reverse phase column by using a method 5 (Column: Redisep Rf Gold ® reversed-phase C18-teledyne ISCO, 50 g ), [Mobile phase A: 10 mM NH4HCO3 in milli-Q water; Mobile phase B: CH3CN) to afford [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)amino]phenyl]methanesulfonate (B-18) (7 mg, 31% yield) as an off-white solid. 10 UPLC-MS (ES+): m z 978.3 [M+H]+. ¹H NMR (400 MHz, DMSO-d6): δ 10.80 (s, 1H), 9.64 (bs, 1H), 8.34 (s, 1H), 7.77 (d, J=8.80 Hz, 1H), 7.65 (dd, J=2.80, 8.80 Hz, 1H), 7.49 (bs, 1H), 7.34-7.30 (m, 2H), 7.08 (bs, 1H), 6.68-6.64 (m, 2H), 6.17 (d, J=7.20 Hz, 1H), 5.36-5.26 (m, 1H), 4.36-4.28 (m, 1H), 4.19-4.11 (m, 3H), 3.71-3.68 (m, 1H), 3.31-3.24 (m, 2H), 3.09-3.01 (m, 2H), 2.81-2.72 (m, 3H), 2.64-2.54 (m, 2H), 2.42-2.32 (m, 1H), 2.16-2.03 (m, 2H), 1.89-1.61 (m, 9H), 1.04 (t, J=7.20 Hz, 3H). Note: Ten aliphatic protons are obscured by the solvent/moisture signals.

Synthesis B19: Synthesis of [2-[1-[2-[(3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]phenyl]methanesulfonate (B-19):

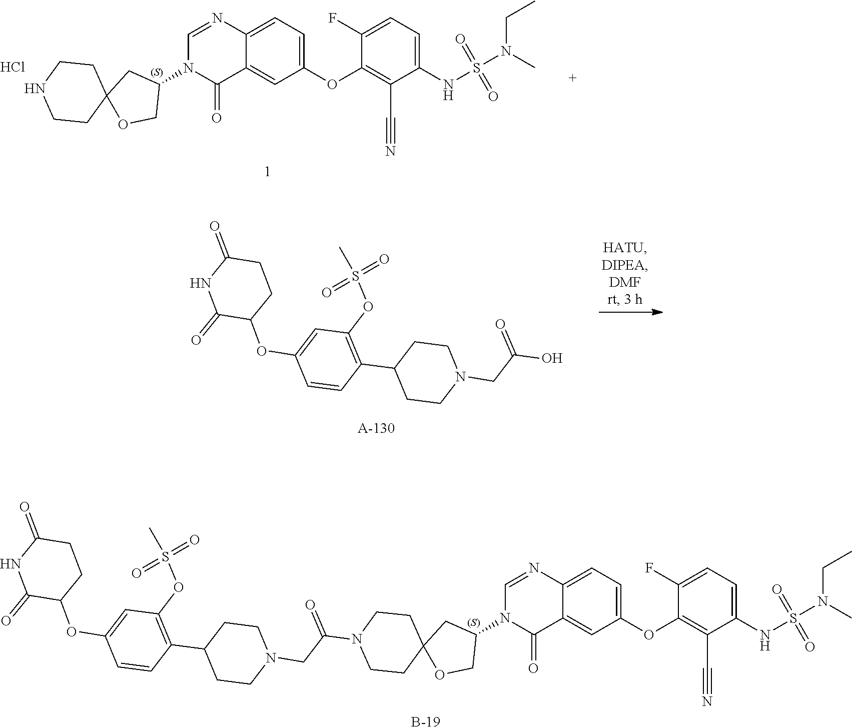

Into a 25 mL single-neck round-bottom flask containing a well-stirred solution of 2-[4-25 [4-[(2,6-dioxo-3-piperidyl)oxy]-2-methylsulfonyloxy-phenyl]-1-piperidyl]acetic acid A-130 (60 mg, 91.27 mol) and (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (2.39 mg, 4.30 mol) in dimethylformamide (2 mL) was added N,N-diisopropylethylamine (365.07 mol, 63.59 L) followed by and HATU (52.05 mg, 136.90 mol) at room temperature. The resulting mixture 5 was stirred at room temperature for 3 hours. Progress of the reaction was monitored by UPLC-MS. After completion of the reaction, the reaction mixture was concentrated under reduced pressure to get crude residue which was purified by reverse phase column by using a method (Column: Redisep Rf Gold®reversed-phase C18-teledyne ISCO,120 g), [Mobile phase A:10 mM NH4HCO3in milli-Q water; Mobile phase B: CH3CN). The fractions were lyophilized to afford [2-[1-[2-[(3S)-3-[6-[2-cyano-3- [[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]phenyl]methanesulfonate (B-19) (30 mg, 33% yield) as a white-solid.

UPLC-MS (ES+): m z 979.4 [M+H]+. ¹H NMR (400 MHz, DMSO-d6): δ 10.98 (s, 1H), 9.90 (bs, 1H), 8.35 (s, 1H), 7.77 (d, J=8.80 Hz, 1H), 7.66 (dd, J=3.20, 9.00 Hz, 1H), 7.54 (bs, 1H), 7.36-7.25 (m, 3H), 7.06-7.03 (m, 2H), 5.35-5.21 (m, 2H), 4.20-4.10 (m, 2H), 3.70-3.65 (m, 1H), 3.60-3.48 (m, 5H), 3.40-3.35 (m, 3H), 3.26-2.85 (m, 5H), 2.77-2.61 (m, 5H), 2.62-2.54 (m, 2H), 2.43-2.35 (m, 1H), 2.28-2.05 (m, 3H), 1.92-1.47 (m, 8H), 1.04 (t, J=7.20 Hz, 3H).

Synthesis B20: Synthesis of [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]phenyl]

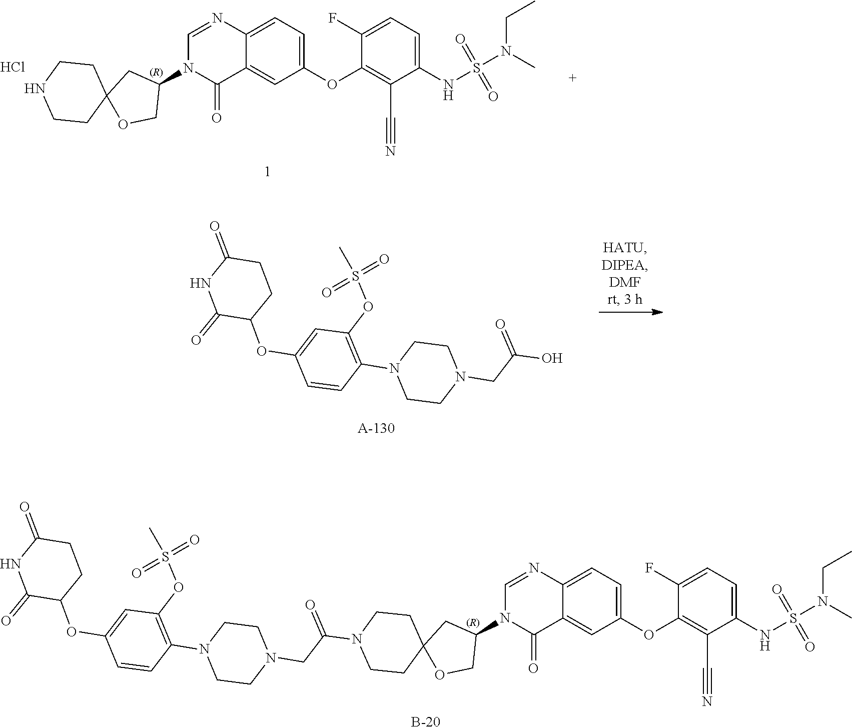

Into a 25 mL single-neck round-bottom flask containing a well-stirred solution of 2-[4-[4-[(2,6-dioxo-3-piperidyl)oxy]-2-methylsulfonyloxy-phenyl]-1-piperidyl]acetic acid A-130 (55 mg, 42.01 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (2.39 mg, 4.30 mol) were in dimethylformamide (2.03 mL) was added N,N-diisopropylethylamine (168.02 mol, 29.27 L) followed by HATU (23.96 mg, 63.01 mol) at room temperature. Resulting reaction mixture was stirred at room temperature for 3 hours. Progress of the reaction was monitored by UPLC-MS. After completion of the reaction, reaction mixture was concentrated directly under reduced pressure to get a crude residue which was purified by reverse phase column by using a method 10 (Column: Redisep Rf Gold®reversed-phase C18-teledyne ISCO,120 g), [Mobile phase A:10 mM NH4HCO3 in milli-Q water; Mobile phase B: CH3CN) to afford [2-[1-[2-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-2-oxo-ethyl]-4-piperidyl]-5-[(2,6-dioxo-3-piperidyl)oxy]phenyl]methanesulfonate (B-20) (4 mg, 9% yield) as an off-white solid. UPLC-MS (ES+): m z 979.7 [M+H]+. ¹H NMR (400 MHz, DMSO-d6): δ 10.97 (s, 1H), 9.77 (bs, 1H), 8.34 (s, 1H), 7.77 (d, J=8.80 Hz, 1H), 7.65 (dd, J=2.80, 8.80 Hz, 1H), 7.40-7.34 (m, 1H), 7.36-7.32 (m, 3H), 7.05-7.00 (m, 2H), 5.40-5.22 (m, 2H), 4.20-4.10 (m, 2H), 3.75-3.65 (m, 1H), 3.60-3.40 (m, 6H), 3.30-2.90 (m, 7H), 2.75-2.60 (m, 5H), 2.50-2.40 (m, 2H), 2.39-2.00 (m, 4H), 1.90-1.40 (m, 8H), 1.03 (t, J=7.20 Hz, 3H).

Synthesis B21: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]piperazin-1-yl]acetyl]-1-oxa-8-azaspiro[4.5]decane (B-21):

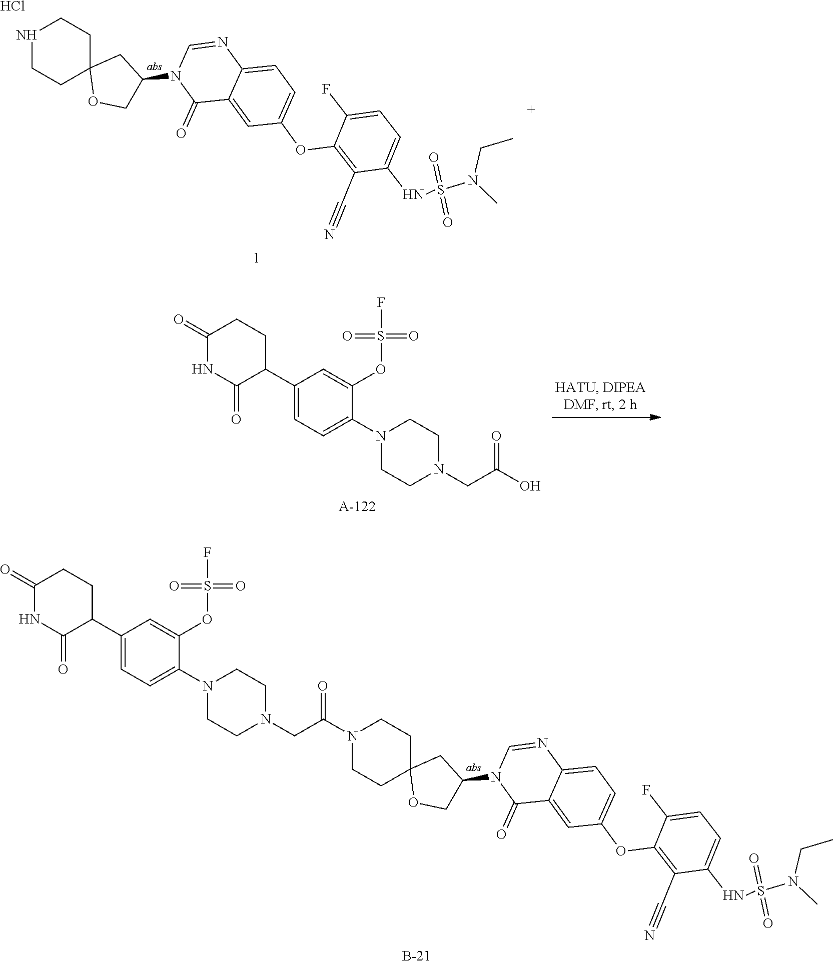

Into a 10 mL single neck round bottom flask containing a well-stirred solution of 2-[4-[4-(2,6-dioxo-3-piperidyl)-2-fluorosulfonyloxy-phenyl]piperazin-1-yl]acetic acid A-122 (49 mg, 86.72 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (51.43 mg, 86.72 mol, HCl salt) in dimethylformamide (1.5 mL) were added N,N-diisopropylethylamine (346.89 mol, 60.42 L) and HATU (65.95 mg, 173.44 mol) at 0° C., stirred at room temperature for 2 hours, The reaction was monitored by UPLC-MS. After completion of the reaction, reaction mixture was concentrated under reduced pressure to get crude which was purified by using RediSep Rf Gold® 60 g column and [Mobile phase A: 10 mM NH4HCO3 in milli-Q water; Mobile phase B: CH3CN], while the desire product was eluting at 25-30% of the mobile phase, the fractions were collected and lyophilized to get (3R)-3-[6-[2-cyano-3-[[ethyl(methyl)sulfamoyl]amino]-6-fluoro-phenoxy]-4-oxo-quinazolin-3-yl]-8-[2-[4-[4-(2,6-dioxo-3-piperidyl)-2-fluorosulfonyloxy-phenyl]piperazin-1-yl]acetyl]-1-oxa-8-azaspiro[4.5]decane (B-21) (39 mg, 46% yield) as an off-white solid. UPLC-MS (ES+): m z 968.5 [M+H]+. ¹H NMR (400 MHz, DMSO-d6): δ 10.86 (s, 1H), 10.16 (bs, 1H), 8.35 (s, 1H), 7.80-7.66 (m, 3H), 7.45-7.41 (m, 2H), 7.36 (d, J=2.80 Hz, 1H), 7.32-7.28 (m, 2H), 5.36-5.25 (m, 1H), 4.15-4.12 (m, 2H), 3.93-3.90 (m, 1H), 3.72-3.45 (m, 4H), 3.13-3.09 (m, 2H), 3.05-2.97 (m, 4H), 2.75-2.70 (m, 4H), 2.66-2.63 (m, 2H), 2.61-2.57 (m, 2H), 2.56-2.54 (m, 2H), 2.50-2.48 (m, 1H), 2.40-2.36 (m, 1H), 2.28-2.21 (m, 1H), 2.12-1.98 (m, 2H), 1.82-1.52 (m, 4H), 1.05 (t, J=7.20 Hz, 3H).

### EXAMPLE 3: SYNTHESIS OF MTH1 DEGRADERS

Synthesis C1: Synthesis 4-[[1-(4-aminobutyl)triazol-4-yl]methoxy]-2-(2,6-dioxo-3-piperidyl)-6-fluorosulfonyloxy-1-oxo-isoindoline (C-1)& 7-((1-(4-(4-(3-(cyclopropylcarbamoyl)-7-fluoro-4-(phenylamino)quinolin-6-yl)-2-fluorobenzamido)butyl)-1H-1,2,3-triazol-4-yl)methoxy)-2-(2,6-dioxopiperidin-3-yl)-3-oxoisoindolin-5-yl sulfurofluoridate (C-2):

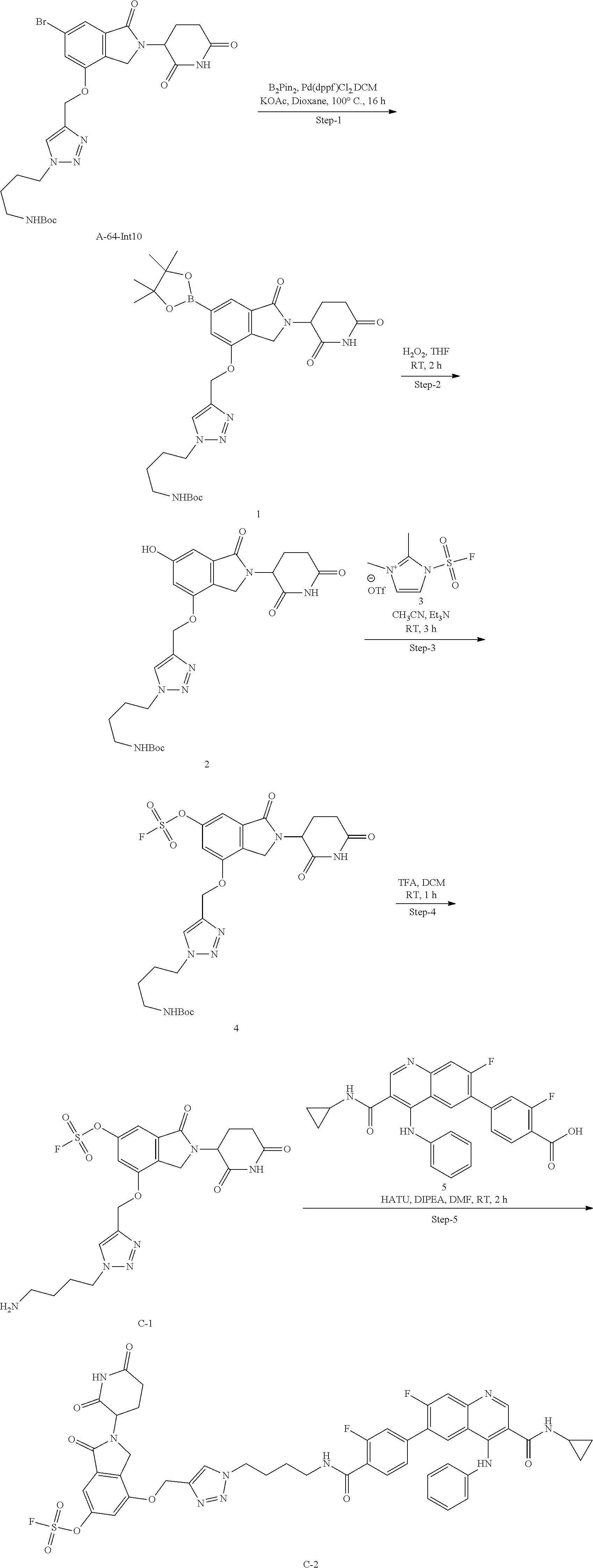

Step-1: To a stirred solution of tert-butyl N-[4-[4-[[6-bromo-2-(2,6-dioxo-3-piperidyl)-1-oxo-isoindolin-4-yl]oxymethyl]triazol-1-yl]butyl]carbamate A-64-Int-10 (0.2 g, 0.338 mmol), 4,4,4′,4′,5,5,5′,5′-Octamethyl-2,2′-bi-1,3,2-dioxaborolane (128.80 mg, 0.507 mmol) in 1,4-dioxane (10 mL). The mixture was degassed with nitrogen for 5 minutes, then were added Pd(dppf)Cl2 DCM (13.81 mg, 0.0169 mmol), potassium acetate (100 mg, 1.01 mmol) and again de-gassed with nitrogen for 5 minutes. The reaction mixture was heated and stirred at 100° C. for 16 hours. After completion, the reaction mixture was concentrate under reduced pressure to afford tert-butyl N-[4-[4-[[2-(2,6-dioxo-3-piperidyl)-1-oxo-6-(4,4,5,5-tetramethyl-1,3,2-dioxaborolan-2-yl)isoindolin-4-yl]oxymethyl]triazol-1-yl]butyl]carbamate 1 (0.25 g, 52% yield) as a yellow solid. LCMS(ES*): m z 639.90 [M+H]+.

Step-2: To a solution of tert-butyl N-[4-[4-[[2-(2,6-dioxo-3-piperidyl)-1-oxo-6-(4,4,5,5-tetramethyl-1,3,2-dioxaborolan-2-yl)isoindolin-4-yl]oxymethyl]triazol-1-yl]butyl]carbamate 1 (0.25 g, 0.391 mmol) in tetrahydrofuran (5 mL) was added 35% hydrogen peroxide (60 L,1.96 mmol) at 0° C. The mixture was stirred at ambient temperature for 2 hours. After completion of the reaction, reaction mixture was filtered over sodium sulfate and washed with tetrahydrofuran (5 mL), the obtained filtrate was concentrated under reduced pressure to afford tert-butyl N-[4-[4-[[2-(2,6-dioxo-3-piperidyl)-6-hydroxy-1-oxo-isoindolin-4-yl]oxymethyl]triazol-1-yl]butyl]carbamate 2 (0.2 g, 50% yield) as a yellow solid. LCMS (ES+): m/z 529.71 [M+H]+.

Step-3: To a stirred solution of tert-butyl N-[4-[4-[[2-(2,6-dioxo-3-piperidyl)-6-hydroxy-1-oxo-isoindolin-4-yl]oxymethyl]triazol-1-yl]butyl]carbamate 2 (0.2 g, 0.378 mmol) in acetonitrile (3.00 mL) were added triethylamine (105 L, 0.756 mmol) and 1-(fluorosulfonyl)-2,3-dimethyl-1H-imidazol-3-ium trifluoromethanesulfonate 3 (187 mg, 0.567 mmol) at room temperature and stirred for 3 hours. After completion, the solvent was removed under reduced pressure to obtain tert-butyl N-[4-[4-[[2-(2,6-dioxo-3-piperidyl)-6-fluorosulfonyloxy-1-oxo-isoindolin-4-yl]oxymethyl]triazol-1-yl]butyl]carbamate 4 (0.3 g, 25% yield) as a brown solid.

LCMS (ES+): m/z 611.58 [M+H]+Step-4: To a stirred solution of tert-butyl N-[4-[4-[[2-(2,6-dioxo-3-piperidyl)-6-fluorosulfonyloxy-1-oxo-isoindolin-4-yl]oxymethyl]triazol-1-yl]butyl]carbamate 4 (0.3 g, 0.491 mmol) in dichloromethane (10 mL) was added trifluoroacetic acid (751.96 L, 9.83 mmol) at 0° C. The reaction mixture was warm to room temperature and stirred for 1 hour.

After completion of the reaction, the reaction mixture was concentrated under reduced pressure to obtain the crude product C-1 (0.250 g, 77% yield, trifluoroacetic acid) {A small portion of crude was purified (~50 mgs) by Prep-HPLC purification to afford 4-[[1-(4-aminobutyl)triazol-4-yl]methoxy]-2-(2,6-dioxo-3-piperidyl)-6-fluorosulfonyloxy-1-oxo-isoindoline (10.5 mg; trifluoroacetic acid) as light brown solid}The left over crude was taken to next reaction without further purification. LCMS (ES*): m z 511.38 [M+H]+.

Prep-HPLC Method: Column/dimensions: X-Bridge C18 (19*250*5 p); Mobile phase A: 0.1% trifluoroacetic acid in water; Mobile Phase B: 100% acetonitrile; Gradient (Time/% B):0/10, 2/10, 9.50/57.1; Flow rate: 18 mL/min; Solubility: acetonitrile+water+tetrahydrofuran ¹H NMR (400 MHz, DMSO-d6) δ: 10.99 (s, 1H), 8.29 (s, 1H), 7.80 (d, J=2.0 Hz, 1H), 7.75-7.50 (m, 3H), 5.38 (s, 2H), 5.15-5.10 (m, 1H), 4.43-4.22 (m, 4H), 2.95-2.74 (m, 3H), 2.60-2.55 (m, 1H), 2.45-2.38 (m, 1H), 2.01-1.98 (m, 1H), 1.91-1.83 (m, 2H), 1.54-1.48 (m, 2H).

Step-5: To a stirred solution of 4-[[1-(4-aminobutyl)triazol-4-yl]methoxy]-2-(2,6-dioxo-3-piperidyl)-6-fluorosulfonyloxy-1-oxo-isoindoline C-1 (0.08 g, 0.156 mmol), 4-[4-anilino-3-(cyclopropylcarbamoyl)-7-fluoro-6-quinolyl]-2-fluoro-benzoic acid 5 (79 mg, 0.172 mmol) in dimethylformamide (2 mL) were added N,N-diisopropylethylamine (136 L, 0.783 mmol), HATU (89.38 mg, 0.235 mmol) at room temperature and stirred for 2 hours. After completion of the reaction, the reaction mixture was quenched with ice-cold water (5 mL), solid fallout in the reaction mixture, which was dried under reduced pressure to obtain the crude product. The crude product was purified by Prep-HPLC. The pure fraction are lyophilized to afford 4-anilino-3-(cyclopropylcarbamoyl)-6-[4-[4-[4-[[2-(2,6-dioxo-3-piperidyl)-6-fluorosulfonyloxy-1-oxo-isoindolin-4-yl]oxymethyl]triazol-1-yl]butylcarbamoyl]-3-fluoro-phenyl]-7-fluoro-quinoline (C-2) (12.1 mg, 8% yield) as a yellow solid. LCMS (ES*): m z 952.57 [M+H]+. ¹H NMR (400 MHz, DMSO-d6): δ 10.98 (s, 1H), 10.65 (brs, 1H), 8.81 (s, 1H), 8.56 (d, J=3.2 Hz, 1H), 8.52-8.40 (m, 2H), 8.31 (s, 1H), 7.85-7.79 (m, 2H), 7.69 (t, J=3.8 Hz, 1H), 7.55 (d, J=2.4 Hz, 1H), 7.45-7.30 (m, 4H), 7.25-7.01 (m, 3H), 5.38 (s, 2H), 5.13-5.09 (m, 1H), 4.50-4.21 (m, 4H), 3.35-3.20 (m, 2H), 2.92-2.86 (m, 1H), 2.65-2.55 (m, 1H), 2.50-2.35 (m, 2H), 2.05-1.80 (m, 3H), 1.60-1.45 (m, 2H), 0.55-0.51 (m, 2H), 0.27 (d, J=2.8 Hz, 2H).

Prep-HPLC Method: Column/dimensions: x-select C18m (19*250*5 p); Mobile phase A: 0.1% trifluoroacetic acid in water; Mobile phase B: 100% acetonitrile; Gradient (Time/% B):0/10, 2/10, 12.5/62.2; Flow rate: 18 mL/min; Solubility: water+acetonitrile Synthesis C₂: Synthesis of 2-((2-(2,6-dioxopiperidin-3-yl)-6-((fluorosulfonyl)oxy)-1-oxoisoindolin-4-yl)oxy)acetic acid (C-3) & 2-(2,6-dioxopiperidin-3-yl)-7-(2-((10-((3-(methylcarbamoyl)-4-(phenylamino) quinolin-6-yl) amino)decyl)amino)-2-oxoethoxy)-3-oxoisoindolin-5-yl sulfurofluoridate (C-4):

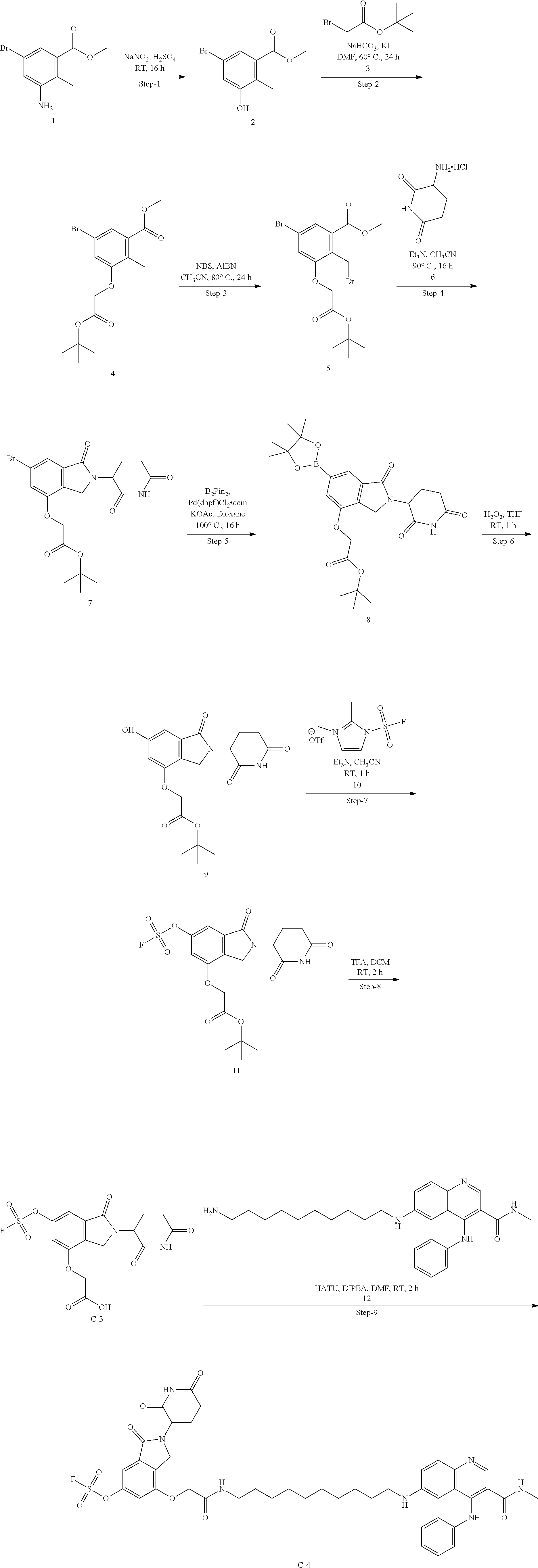

To a stirred solution of methyl 3-amino-5-bromo-2-methyl-benzoate 1 (30 g, 122.91 5 mmol) in con. sulfuric acid (65 mL) was added sodium nitrite (10.18 g, 147.49 mmol) in portions at 0° C. The resulting mixture was stirred at room temperature for 16 hours. After completion of the reaction, the resulting mixture was diluted with water (200 mL) and extracted with ethyl acetate (3×300 mL). The combined organic layer was washed with saturated sodium chloride solution (300 mL) and dried over anhydrous sodium sulfate, filtrated and concentrated 10 under reduced pressure to afford crude product. The crude product was purified by flash column chromatography (Davisil silica) using -10% ethyl acetate in petroleum ether as an eluent to afford methyl 5-bromo-3-hydroxy-2-methyl-benzoate 2 (16.5 g, 52% yield) as a light-yellow solid. LCMS (ES-): m z 245.08 [M-H]-.

Step-2: To a solution of methyl 5-bromo-3-hydroxy-2-methyl-benzoate 2 (7.5 g, 30.60 mmol) in dimethylformamide (80 mL) were added KI (2.54 g, 15.30 mmol), sodium bicarbonate (5.14 g, 61.21 mmol) followed by tert-butyl 2-bromoacetate 3 (8.98 mL, 61.21 mmol) at room temperature and stirred at 60° C. for 24 hours. After completion of the reaction, the reaction mixture was diluted with water (100 mL) and extracted with ethyl acetate (3×200 mL). The 20 combined organic layer was washed with saturated sodium chloride solution (100 mL), dried over sodium sulfate and concentrated under reduced pressure to afford methyl 5-bromo-3-(2-tert-butoxy-2-oxo-ethoxy)-2-methyl-benzoate 4 (6.0 g, 47% yield) as light-yellow solid.

LCMS (ES*): m z 382.45 [M+Na]f.

Step-3: To a stirred solution of methyl 5-bromo-3-(2-tert-butoxy-2-oxo-ethoxy)-2-methyl-benzoate 4 (6 g, 16.70 mmol) in CCl₄ (60 mL) was added N-bromosuccinimide (3.57 g, 20.04 mmol) at room temperature and stirred at 80° C. for 24 hours. After completion of the reaction, the reaction mixture was filtered through celite bed and washed with carbon tetrachloride (50 mL). The obtained filtrate was concentrated under reduced pressure to afford methyl 5-bromo-2-(bromomethyl)-3-(2-tert-butoxy-2-oxo-ethoxy) benzoate 5 (8 g, 65% yield) as brown color oil. ¹H NMR (400 MHz, DMSO-d6): δ 7.59 (d, J=2.0 Hz, 1H), 7.28 (d, J=2.0 Hz, 1H), 4.94 (s, 2H), 4.82 (s, 2H), 3.88 (s, 3H), 1.42 (s, 9H).

Step-4: To a stirred solution of methyl 5-bromo-2-(bromomethyl)-3-(2-tert-butoxy-2-oxo-ethoxy)benzoate 5 (8.0 g, 18.26 mmol), 3-aminopiperidine-2,6-dione 6 (4.51 g, 27.39 mmol, 15 Hydrochloric acid) in acetonitrile (50 mL) was added triethylamine (7.64 mL, 54.78 mmol) and stirred at 90° C. for 16 hours. After completion of reaction, the reaction mixture was concentrated under reduced pressure to get the crude product. The crude product was suspended in (1:1) ratio of ethyl acetate and water (50 mL) to obtain precipitate. The obtained precipitate was filtered and washed with diethyl ether (50 mL) to afford tert-butyl 2-[6-bromo-2-(2,6-dioxo-3-piperidyl)-1-oxo-isoindolin-4-yl]oxyacetate 7 (4 g, 36% yield) as a purple solid. LCMS (ES+): m z 397.30 [M-^{t}Bu+H]+.

Step-5: To a stirred solution of tert-butyl 2-[6-bromo-2-(2,6-dioxo-3-piperidyl)-1-oxo-isoindolin-4-yl]oxyacetate 7 (0.7 g, 1.54 mmol) and 4,4,5,5-tetramethyl-2-(4,4,5,5-tetramethyl-1,3,2-dioxaborolan-2-yl)-1,3,2-dioxaborolane (588.23 mg, 2.32 mmol) in 1,4-dioxane (8 mL) added potassium acetate (454 mg, 4.63 mmol) and degassed with N2 gas for 5 minutes before adding Pd(dppf)Cl2 DCM (100.89 mg, 0.123 mmol) and stirred at 100° C. for 16 hours. After completion of the reaction, the reaction mixture was filtered through celite bed, washed with ethyl acetate (20 mL) and the filtrate was concentrated under reduced pressure to obtain crude product. The crude was purified by flash column chromatography (over 230-400 silica, using 50% ethyl acetate in petroleum ether) to afford tert-butyl 2-[2-(2,6-dioxo-3-piperidyl)-1-oxo-6-(4,4,5,5-tetramethyl-1,3,2-dioxaborolan-2-yl)isoindolin-4-yl]oxyacetate 8 (0.4 g, 19% yield) as a brown solid. LC-MS (ES+): m z 501.43 [M+H]+. Step-6: To a stirred solution of tert-butyl 2-[2-(2,6-dioxo-3-piperidyl)-1-oxo-6-(4,4,5,5-tetramethyl-1,3,2-dioxaborolan-2-yl)isoindolin-4-yl]oxyacetate 8 (0.4 g, 0.799 mmol) in tetrahydrofuran (8 mL) was added 30% hydrogen peroxide in water (0.26 mL) at 0° C., the reaction mixture was stirred at room temperature for 1 hour. After completion of the reaction, the reaction mixture was quenched with saturated sodium bisulfite solution (5 mL) and extracted with ethyl acetate (2×20 mL). The combined organic layer was dried over anhydrous sodium sulfate, filtered and evaporated under reduced pressure to afford tert-butyl 2-[2-(2,6-dioxo-3-piperidyl)-6-hydroxy-1-oxo-isoindolin-4-yl]oxyacetate 9 (0.3 g, 36% yield) as a brown semi-solid. LC-MS (ES*): m z 391.53 [M+H]+.

Step-7: To a stirred solution of tert-butyl 2-[2-(2,6-dioxo-3-piperidyl)-6-hydroxy-1-oxo-isoindolin-4-yl]oxyacetate 9 (0.3 g, 0.768 mmol) in acetonitrile (4 mL) were added triethylamine (0.160 mL, 1.15 mmol) and 1-(fluorosulfonyl)-2,3-dimethyl-1H-imidazol-3-ium trifluoromethanesul-fonate 10 (379.55 mg, 1.15 mmol) at room temperature and continued the reaction for 1 hour. After completion of the reaction, the solvent was evaporated under reduced pressure to afford crude product tert-butyl 2-[2-(2,6-dioxo-3-piperidyl)-6-fluorosulfonyloxy-1-oxo-isoindolin-4-yl]oxyacetate 11 (0.4 g, 42% yield) as brown liquid. LC-MS (ES+): m z 471.44 [M+H]+.

Step-8: To a stirred solution of tert-butyl 2-[2-(2,6-dioxo-3-piperidyl)-6-fluorosulfonyloxy-1-oxo-isoindolin-4-yl]oxyacetate 11 (0.4 g, 0.846 mmol) in dichloromethane (10 mL) was added trifluoroacetic acid (1 mL, 13.07 mmol) at 0° C. and stirred at room temperature for 2 hours.

After completion of the reaction, the reaction mixture was concentrated under reduced pressure, triturated with diethyl ether (3×10 mL) and dried under reduced pressure to obtain crude product (0.3 g, 85% yield) {A small portion of crude was purified (-60 mgs) by Prep-HPLC purification to afford 2-[2-(2,6-dioxo-3-piperidyl)-6-fluorosulfonyloxy-1-oxo-isoindolin-4-yl]oxyacetic acid C-3 (13 mg) as an off-white solid}. The left-over crude was taken to next reaction without further purification. LC-MS (ES+): m z 417.25 [M+H]+. ¹H NMR (400 MHz, DMSO-d6) δ: 13.24 (s, 1H), 11.01 (s, 1H), 7.59 (s, 1H), 7.5 (s, 1H), 5.15-5.11 (dd, J 18 Hz, 1H), 4.94 (s, 2H), 4.49-4.30 (m, 2H), 2.95-2.86 (m, 1H), 2.61-2.41 (m, 2H), 2.02 (t, J 13.2 Hz, 1H).

Prep HPLC method: Column/dimensions: X-select C18 (19*250*5 pm); Mobile phase A: 0.1% trifluoroacetic acid in water; Mobile phase B: 100% acetonitrile; Gradient (Time/% B): 0/10, 2/10, 15/95; Flow rate: 18 mL/min; Solubility: water+acetonitrile Step-9: To a solution of 6-(10-aminodecylamino)-4-anilino-N-methyl-quinoline-3-carboxamide 12 (0.1 g, 0.178 mmol, trifluoroacetic acid) and 2-[2-(2,6-dioxo-3-piperidyl)-6-fluorosulfonyloxy-1-oxo-isoindolin-4-yl]oxyacetic acid C-3 (74 mg, 0.178 mmol) in dimethylformamide (2 mL) was added N,N-diisopropylethylamine (155 L, 0.890 mmol) followed by HATU (102 mg, 0.267 mmol) at room temperature. The reaction mixture was stirred at room temperature for 2 hours. After completion of the reaction, the reaction mixture was diluted with ice-cold water (5 mL), the precipitate obtained was filtered and dried under reduced pressure to afford the crude product, which was purified by MS-prep HPLC to afford 4-anilino-6-[10-[[2-[2-(2,6-dioxo-3-piperidyl)-6-fluorosulfonyloxy-1-oxo-isoindolin-4-yl]oxyacetyl]amino]decylamino]-3-(methylcarbamoyl)quinolone C-4 (7.5 mg, 5% yield) as a yellow solid. LC-MS (ES*): m z 846.62 [M+H]+. ¹H NMR (400 MHz, DMSO-d6): δ 11.04 (s, 1H), 10.85 (brs, 1H), 8.58 (s, 2H), 8.11 (t, J 5.8 Hz, 1H), 7.64 (d, J 9.2 Hz, 1H), 7.56 (d, J=1.6 Hz, 1H), 7.44 (d, J 1.6 Hz, 1H), 7.21 (t, J=7.8 Hz, 2H), 7.14 (d, J=9.6 Hz, 1H), 6.94 (t, J=6.8 Hz, 1H), 6.86 (d, J 7.2 Hz, 2H), 6.42 (s, 1H), 6.12 (s, 1H), 5.17-5.12 (m, 1H), 4.76 (s, 2H), 4.46 (m, 2H), 3.19-3.05 (m, 2H), 2.96-2.80 (m, 1H), 2.69-2.38 (m, 7H), 2.01-1.90 (m, 1H), 1.41-1.16 (m, 16H).

Prep HPLC method: Column/dimensions: X-BRIDGE C18(19*250); Mobile phase A: 0.1% trifluoroacetic acid in Water Mobile Phase B: 100% acetonitrile; Gradient (Time/% B):0/10, 2/30, 11.10/75, 11.20/98, 13.50/98, 13.60/10, 16.50/10; Solubility: water+acetonitrile+tetrahydrofuran Synthesis C₃: Synthesis of 7-((1-(4-(4-(3-(cyclopropylcarbamoyl)-7-fluoro-4-(phenylamino)quinolin-6-yl)-2-fluorobenzamido)butyl)-1H-1,2,3-triazol-4-yl)methoxy)-2-(2,6-dioxopiperidin-3-yl)-3-oxoisoindoline-5-sulfonyl fluoride (C-5):

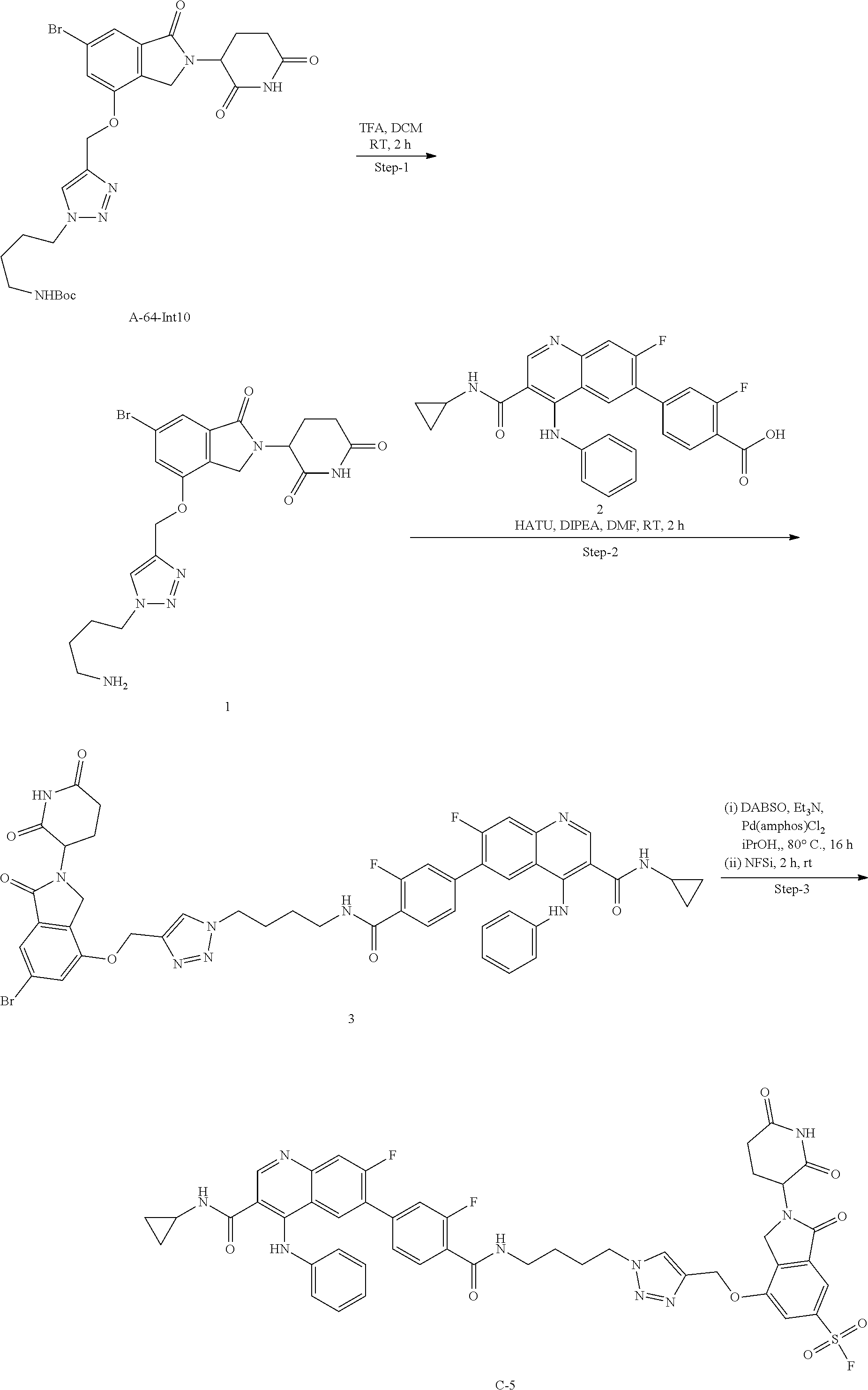

Step-1: To a stirred solution of tert-butyl N-[4-[4-[[6-bromo-2-(2,6-dioxo-3-piperidyl)-1-oxo-isoindolin-4-yl]oxymethyl]triazol-1-yl]butyl]carbamate A-64-Int-10 (0.4 g, 0.676 mmol) in 10 dichloromethane (10 mL) was added trifluoroacetic acid (1.04 mL, 13.53 mmol) at 0° C. The reaction mixture was warm to room temperature and continued stirring for 2 hours. After completion, the reaction mixture was concentrated under reduced pressure to obtain crude residue, which was triturated with diethyl ether (3×10 mL) dried under reduced pressure to afford 3-[4-[[1-(4-aminobutyl)triazol-4-yl]methoxy]-6-bromo-1-oxo-isoindolin-2-yl]piperidine-2,6-dione 1 (0.33 g, 53% yield, trifluoroacetic acid) as a yellow solid. LCMS (ES*): m z 493.51 [M+H]+.

Step-2: To a stirred solution of 3-[4-[[1-(4-aminobutyl)triazol-4-yl]methoxy]-6-bromo-1-oxo-isoindolin-2-yl]piperidine-2,6-dione 1 (0.11 g, 0.182 mmol, Trifluoroacetic acid), 4-[4-anilino-3-(cyclopropylcarbamoyl)-7-fluoro-6-quinolyl]-2-fluoro-benzoic acid 2 (91.83 mg, 0.199 mmol) in dimethylformamide (2 mL) were added N,N-diisopropylethylamine (95 L, 0.545 mmol) followed by HATU (103.64 mg, 0.272 mmol) at room temperature and stirred for 2 hours. After completion of the reaction, the reaction mixture was diluted with ice-cold water (5 mL) to obtain precipitate, which was filtered and washed with water (10 mL), dried under reduced pressure to afford 4-anilino-6-[4-[4-[4-[[6-bromo-2-(2,6-dioxo-3-piperidyl)-1-oxo-isoindolin-4-yl]oxymethyl]triazol-1-yl]butylcarbamoyl]-3-fluoro-phenyl]—N-cyclopropyl-7-fluoro-quinoline-3-carboxamide 3 (0.12 g, 37% yield) as yellow solid. LCMS (ES+): m z 934.65 [M+H]+.

Step-3: To a stirred solution of DABCO(SO2)2 (15.46 mg, 0.0643 mmol), 4-anilino-6-[4-[4-[4-[[6-bromo-2-(2,6-dioxo-3-piperidyl)-1-oxo-isoindolin-4-yl]oxymethyl]triazol-1-yl]butylcarbamoyl]-3-fluoro-phenyl]—N-cyclopropyl-7-fluoro-quinoline-3-carboxamide 3 (0.1 g, 0.107 mmol) in a mixture of DMSO (0.5 mL), isopropanol (3 mL) and triethylamine (45 L, 321.62 mol) was purged with nitrogen for 5 minutes before adding Pd(amphos)C12 (3.80 mg, 0.0053 mmol) and stirred at 80° C. for 16 hours. After cooling to room temperature N-fluorobenzenesulfonimide (50.71 mg, 0.160 mmol) was added and stirred for 2 hours. The reaction mixture was concentrated under reduced pressure to obtain crude product, which was purified by Prep-HPLC purification. The pure fractions are lyophilized to afford 7-[[1-[4-[[4-[4-anilino-3-(cyclopropylcarbamoyl)-7-fluoro-6-quinolyl]-2-fluoro-benzoyl]amino]butyl]triazol-4-yl]methoxy]-2-(2,6-dioxo-3-piperidyl)-3-oxo-isoindoline-5-sulfonyl fluoride C-5 (9.2 mg, 8% yield) as a yellow solid. LCMS (ES+): m z 936.60 [M+H]+. H-NMR (400 MHz, DMSO-d6): δ 11.01 (s, 1H), 10.80 (br s, 1H), 8.81 (s, 1H), 8.57 (d, J=3.2 Hz, 2H), 8.49-8.40 (m, 1H), 8.30 (s, 1H), 8.14 (s, 1H), 7.94 (s, 1H), 7.85 (d, J=11.2 Hz, 1H), 7.70 (t, J=8.0 Hz, 1H), 7.50-7.35 (m, 4H), 7.30-7.01 (m, 3H), 5.52 (s, 2H), 5.17-5.12 (m, 1H), 4.60-4.35 (m, 4H), 3.35-3.20 (m, 2H), 2.95-2.80 (m, 1H), 2.60-2.55 (m, 1H), 2.45-2.30 (m, 2H), 2.05-1.80 (m, 3H), 1.60-1.45 (m, 2H), 0.60-0.45 (m, 2H), 0.30-0.20 (m, 2H).

Prep-HPLC Method: Column/dimensions: X-Bridge C18(19*250*5 p); Mobile phase A: 0.1% trifluoroacetic acid in water; Mobile phase B: 100% acetonitrile; Gradient (Time/% B) :0/10, 2/10,10/55, 12.30/55, 12.40/98, 15/98, 15.10/10, 17/10; Flow rate: 18 mL/min; Solubility: acetonitrile+water+tetrahydrofuran Synthesis C₄: Synthesis of 2-(2,6-dioxopiperidin-3-yl)-7-(2-((4-(5-(3-(methylcarbamoyl)-4-(phenylamino)quinolin-6-yl)picolinamido)butyl)amino)-2-oxoethoxy)-3-oxoisoindolin-5-yl methanesulfonate (C-6):

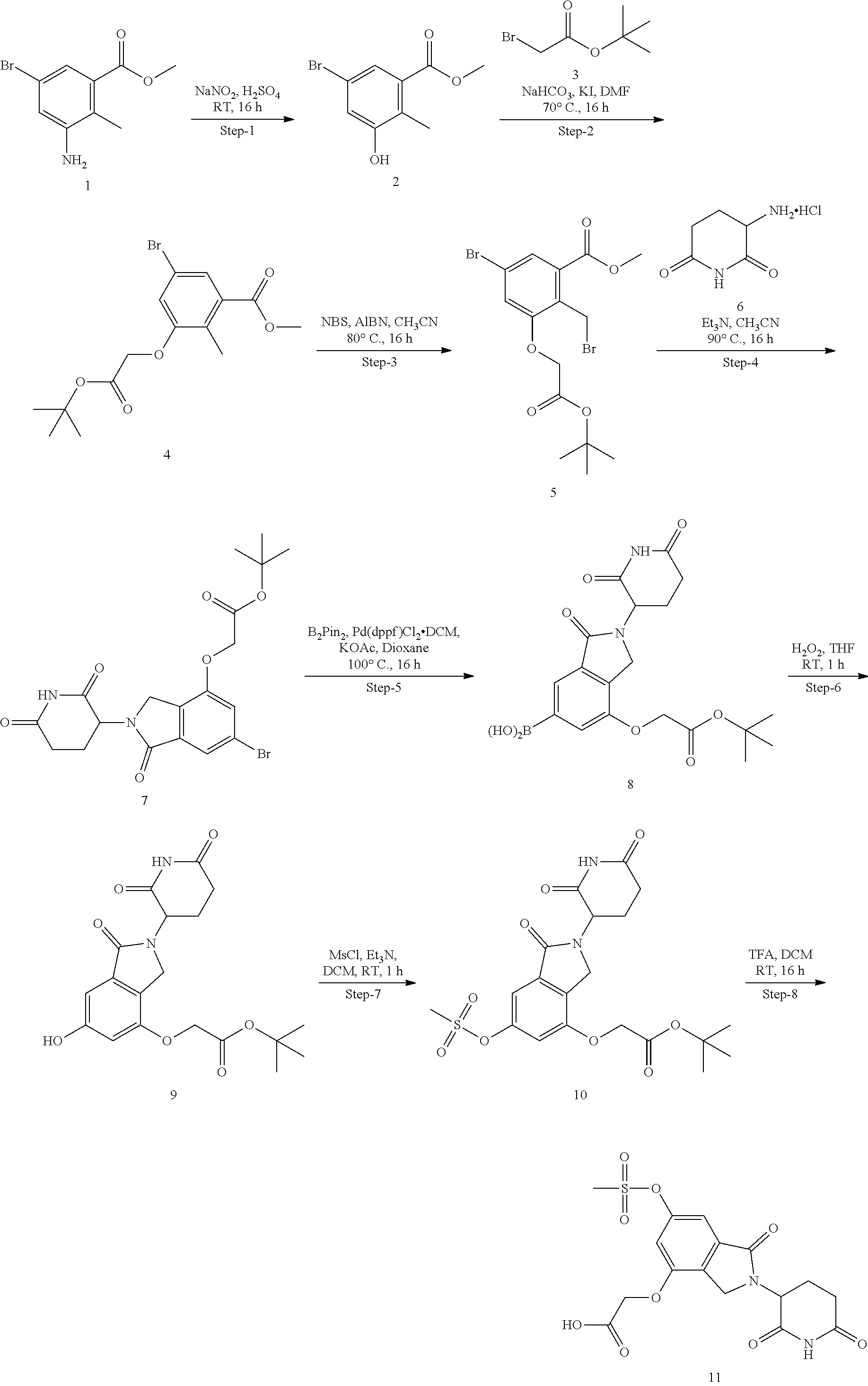

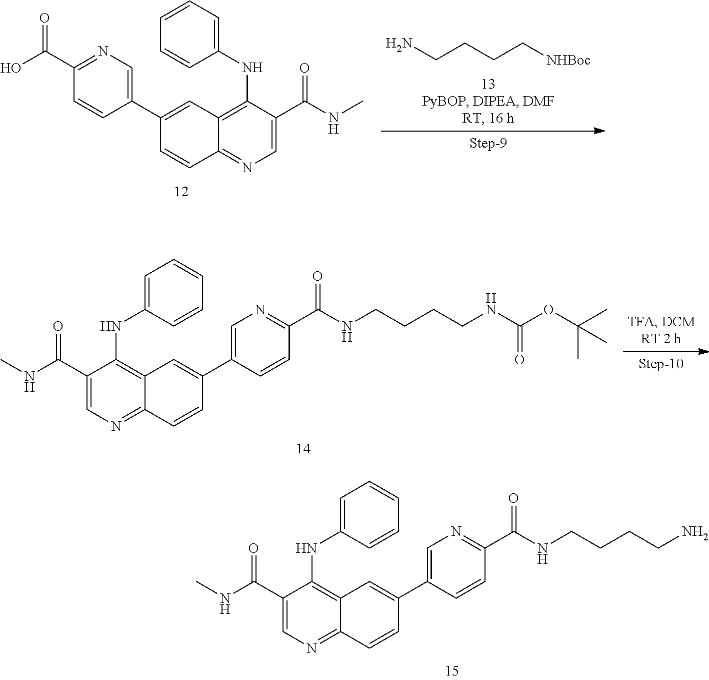

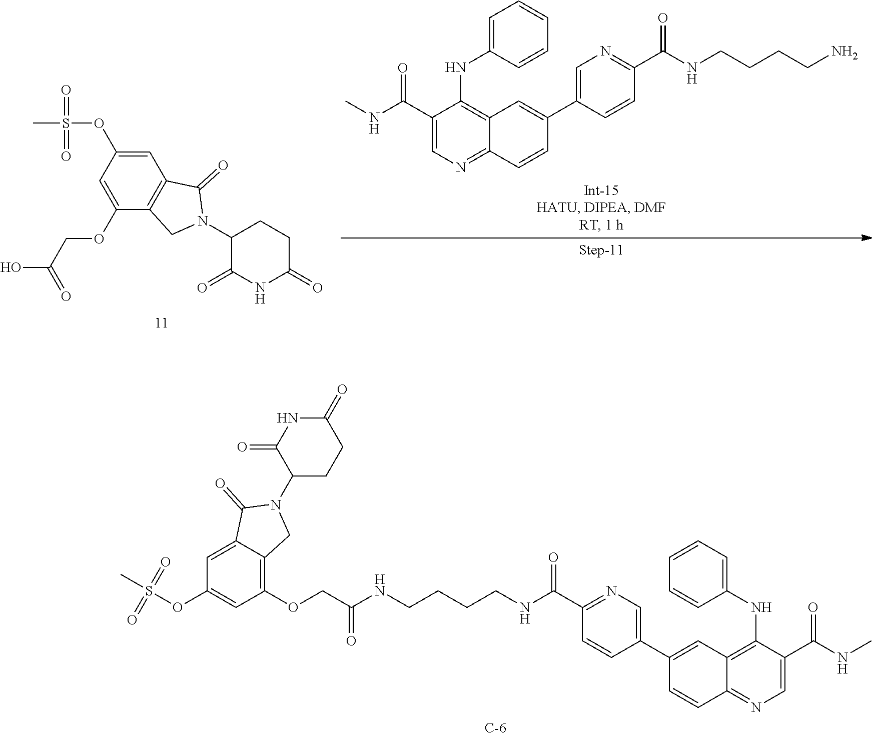

Step-i: To a stirred solution of methyl 3-amino-5-bromo-2-methyl-benzoate 1 (20 g, 81.94 mmol) in con. sulfuric acid (80 mL) was added NaNO2 (6.80 g, 98.56 mmol) in portions at 0° C. The resulting mixture was stirred at room temperature for 16 hours. After completion of the reaction, the resulting mixture was diluted with water (200 mL) and extracted with ethyl 10 acetate (3×300 mL). The combined organic layers were washed with saturated sodium chloride solution (300 mL) and dried over sodium sulfate, concentrated under reduced pressure to afford crude product, which was purified by flash column chromatography (Davisil silica) using 20% ethyl acetate in petroleum ether to afford methyl 5-bromo-3-hydroxy-2-methyl-benzoate 2 (11.6 g, 40% yield) as a brown solid. LCMS (ES*): m z 245.20 [M+H]+. Step-2: To a stirred solution of methyl 5-bromo-3-hydroxy-2-methyl-benzoate 2 (1 g, 4.08 mmol) in dimethylformamide (10 mL) was added KI (338 mg, 2.04 mmol) and sodium bicarbonate (685 mg, 8.16 mmol) at room temperature and stirred 5 minutes. Tert-butyl 2-bromoacetate 3 (1.20 mL, 8.16 mmol) was added into the reaction mixture and stirred for 16 hours at 70° C. After completion of the reaction, the reaction mixture was diluted with ice-cold water (20 mL) and extracted with ethyl acetate (3×15 mL). The combined organic layer was dried over anhydrous sodium sulfate and evaporated under reduced pressure to get the crude product. The crude product was purified by column chromatography (Davisil silica) using 60% 10 ethyl acetate in petroleum ether as an eluent to afford methyl 5-bromo-3-(2-tert-butoxy-2-oxo-ethoxy)-2-methyl-benzoate 4 (1.45 g, 82% yield) as pale-yellow liquid. LC-MS (ES*): m z 304.86 [M-^{t}Bu+H]+.

Step-3: To a stirred solution of methyl 5-bromo-3-(2-tert-butoxy-2-oxo-ethoxy)-2-methyl-benzoate 4 (1.45 g, 4.04 mmol) in CCl₄ (20 mL) was added N-bromosuccinimide (862 mg, 4.84 mmol) at room temperature and stirred for 5 minutes. AIBN (66 mg, 0.403 mmol) was added into the reaction mixture at 0° C. and stirred for 16 hours at 80° C. After completion of the reaction, the reaction mixture was filtered through celite bed and washed with carbon tetrachloride (30 mL). The obtained filtrate was concentrated under reduced pressure to obtain the crude product methyl 5-bromo-2-(bromomethyl)-3-(2-tert-butoxy-2-oxo-ethoxy) benzoate 5 (1.75 g, 79% yield) as pale-yellow liquid. H-NMR (400 MHz, DMSO-d6): δ 7.72 (d, J=1.60 Hz, 1H), 7.05 (d, J=1.60 Hz, 1H), 5.05 (s, 2H), 4.62 (s, 2H), 3.95 (s, 3H), 1.50 (s, 9H).

Step-4: To a stirred solution of methyl 5-bromo-2-(bromomethyl)-3-(2-tert-butoxy-2-oxo-ethoxy) benzoate 5 (1.75 g, 3.99 mmol) in acetonitrile (10 mL) were added triethylamine (1.67 mL, 11.98 mmol), 3-aminopiperidine-2,6-dione 6 (614 mg, 4.79 mmol) and stirred for 16 hours at 90° C. After completion of the reaction, the reaction mixture was evaporated under reduced pressure to get the crude product. The crude product was triturated with diethyl ether (20 mL), and washed with dichloromethane (10 mL) to afford tert-butyl 2-[6-bromo-2-(2,6-dioxo-3-piperidyl)-1-oxo-isoindolin-4-yl]oxyacetate 7 (1.8 g, 66% yield) as a dark blue solid. LC-MS (ES-): m z 452.92 [M-H]-.

Step-5: To a stirred solution of tert-butyl 2-[6-bromo-2-(2,6-dioxo-3-piperidyl)-1-oxo-isoindolin-4-yl]oxyacetate 7 (0.5 g, 1.10 mmol) and 4,4,5,5-tetramethyl-2-(4,4,5,5-tetramethyl-1,3,2-dioxaborolan-2-yl)-1,3,2-dioxaborolane (420 mg, 1.65 mmol) in dioxane (10 mL) was added potassium acetate (325 mg, 3.31 mmol) and degassed with argon gas for 10 minutes. Pd(dppf)C12 DCM (45 mg, 0.055 mmol) was added into the reaction mixture and stirred at 100° C. for 16 hours. After completion of the reaction, the reaction mixture was filtered through celite bed and washed with dichloromethane (30 mL). The obtained filtrate was concentrated under reduced pressure to obtain the crude product [7-(2-tert-butoxy-2-oxo-ethoxy)-2-(2,6-dioxo-3-piperidyl)-3-oxo-isoindolin-5-yl]boronic acid 8 (0.5 g, 23% yield) as reddish-brown oil, which was used directly in the next step without further purification. LC-MS (ES*): m z 419.42 [M+H]+.

Step-6: To a stirred solution of [7-(2-tert-butoxy-2-oxo-ethoxy)-2-(2,6-dioxo-3-piperidyl)-3-oxo-isoindolin-5-yl]boronic acid 8 (0.5 g, 1.20 mmol) in tetrahydrofuran (8 mL) was added 35% hydrogen peroxide in water (0.36 mL, 11.96 mmol) at 0° C. and stirred at room temperature for 1 hour. After completion, the reaction mixture was quenched with saturated sodium bisulfate (10 mL) and extracted with ethyl acetate (2×20 mL). The combined organic layer was dried over anhydrous sodium sulfate and evaporated under reduced pressure to obtain the crude product tert-butyl 2-[2-(2,6-dioxo-3-piperidyl)-6-hydroxy-1-oxo-isoindolin-4-yl]oxyacetate 9 (0.2 g, 12% yield) as reddish-brown semi solid, which was used directly in the next step without further purification. LC-MS (ES+): m z 391.35 [M+H]+Step-7: To a stirred solution of tert-butyl 2-[2-(2,6-dioxo-3-piperidyl)-6-hydroxy-1-oxo-isoindolin-4-yl]oxyacetate 9 (0.2 g, 0.512 mmol) in dichloromethane (3 mL) was added triethylamine (0.142 mL, 1.02 mmol) and stirred for 5 minutes. Then methane sulfonyl chloride (0.047 mL, 0.614 mmol) was added into the reaction mixture at 0° C. and stirred for 1 hour at room temperature. After completion of the reaction, the reaction mixture was diluted with saturated sodium bicarbonate solution (20 mL) and extracted with dichloromethane (20 mL×2).

The combined organic layer was dried over anhydrous sodium sulfate and evaporated under reduced pressure to obtain the crude product tert-butyl 2-[2-(2,6-dioxo-3-piperidyl)-6-methylsulfonyloxy-1-oxo-isoindolin-4-yl]oxyacetate 10 (0.1 g, 7% yield) as reddish-brown semi-solid, which was used directly in the next step without further purification. LC-MS (ES+): m z 469.42 [M+H]+.

Step-8: To a stirred solution of tert-butyl 2-[2-(2,6-dioxo-3-piperidyl)-6-methylsulfonyloxy-1-oxo-isoindolin-4-yl]oxyacetate 10 (0.1 g, 0.213 mmol) in dichloromethane (2 mL) were added trifluoroacetic acid (0.196 mL, 2.56 mmol) at 0° C. The reaction mixture was stirred at room temperature for 16 hours. After completion of the reaction, the reaction mixture was concentrated under reduced pressure to obtain crude product. The crude product was triturated with diethyl ether (20 mL) to afford 2-[2-(2,6-dioxo-3-piperidyl)-6-methylsulfonyloxy-1-oxo-isoindolin-4-yl]oxyacetic acid 11 (0.08 g, 16% yield) as a pale-brown solid. LC-MS (ES*): m/z 413.05 [M+H]+.

Step-9: To a stirred solution of 5-[4-anilino-3-(methylcarbamoyl)-6-quinolyl]pyridine-2-carboxylic acid 12 (0.3 g, 0.752 mmol) and tert-butyl N-(4-aminobutyl)carbamate 13 (212 mg, 1.13 mmol) in dimethylformamide (5 mL) was added N,N-diisopropylethylamine (0.4 mL, 2.26 mmol) at room temperature and stirred for 5 minutes. PyBOP (588 mg, 1.13 mmol) was added into the reaction mixture and stirred for 16 hours at room temperature. After completion of the reaction, the reaction mixture was quenched with ice-cold water (10 mL). The obtained solid was filtered to obtain the crude product tert-butyl N-[4-[[5-[4-anilino-3-(methyl carbamoyl)-6-quinolyl]pyridine-2-carbonyl]amino]butyl]carbamate 14 (0.32 g, 61% yield) as an off-white solid, which was used directly in the next step without further purification. LC-MS (ES+): m z 569.41 [M+H]+.

Step-10: To a stirred solution of tert-butyl N-[4-[[5-[4-anilino-3-(methyl carbamoyl)-6-quinolyl]pyridine-2-carbonyl]amino]butyl]carbamate 14 (0.32 g, 0.562 mmol) in dichloromethane (5 mL) were added trifluoroacetic acid (0.516 mL, 6.75 mmol) at 0° C. The reaction mixture was stirred at room temperature for 2 hours. After completion of the reaction, the reaction mixture was concentrated under reduced pressure to obtain crude product. The crude product was triturated with diethyl ether (20 mL) to afford 6-[6-(4-aminobutylcarbamoyl)-3-pyridyl]-4-anilino-N-methyl-quinoline-3-carboxamide 15 (0.3 g, 86% yield) as a pale-brown solid. LC-MS (ES+): m z 469.49 [M+H]+.

Step-11: To a stirred solution of 6-[6-(4-aminobutylcarbamoyl)-3-pyridyl]-4-anilino-N-methyl-quinoline-3-carboxamide 11 (80 mg, 0.137 mmol) and 2-[2-(2,6-dioxo-3-piperidyl)-6-methylsulfonyloxy-1-oxo-isoindolin-4-yl]oxyacetic acid Int-15 (87 mg, 0.164 mmol) in dimethylformamide (2 mL) was added N,N-diisopropylethylamine (0.071 mL, 0.411 mmol) at room temperature and stirred for 5 minutes. HATU (78 mg, 0.205 mmol) was added into the reaction mixture and stirred for 1 hour at room temperature. After completion of the reaction, the reaction mixture was purified by MS Prep HPLC to afford [7-[2-[4-[[5-[4-anilino-3-(methylcarbamoyl)-6-quinolyl]pyridine-2-carbonyl]amino]butylamino]-2-oxo-ethoxy]-2-(2,6-dioxo-3-piperidyl)-3-oxo-isoindolin-5-yl]methane sulfonate C-6 (20.2 mg, 17% yield) as a pale-yellow solid. LC-MS (ES*): m z 863.46 [M+H]+. ¹H NMR (400 MHz, DMSO-d6) δ: 11.01 (s, 2H), 8.93 (s, 1H), 8.83-8.76 (m, 3H), 8.48 (d, J=4.40 Hz, 1H), 8.40 (d, J=4.40 Hz, 1H), 8.31 (d, J=4.40 Hz, 1H), 8.15-8.10 (m, 3H), 7.43 (t, J=7.60 Hz, 2H), 7.31-7.08 (m, 5H), 5.16 (dd, J=5.20 Hz, 1H), 4.68 (s, 2H), 4.50 (s, 1H), 4.39 (s, 1H), 3.42 (s, 3H), 3.32 (s, 2H), 3.18-3.13 (m, 2H), 2.96-2.90 (m, 1H), 2.60 (s, 1H), 2.44-2.38 (m, 4H), 2.02-1.99 (m, 1H), 1.56-1.47 (m, 4H).

Prep HPLC method: Column/dimensions: X SELECT C18 (19*250*5 um); Mobile phase A: 0.1% trifluoroacetic acid in water (aq); Mobile phase B: acetonitrile (org); Gradient (Time/% B): 0/16, 2/16, 10/33, 11.8/33, 11.9/100, 15.5/100, 15.6/16, 18.5/16. Flow rate: 18 mL/min Solubility: acetonitrile+tetrahydrofuran+Water Synthesis C₅: Synthesis of 2-(2,6-dioxopiperidin-3-yl)-7-(2-((4-(5-(3-(methylcarbamoyl)-4-(phenylamino)quinolin-6-yl)picolinamido)butyl)amino)-2-oxoethoxy)-1,3-dioxoisoindolin-5-yl methanesulfonate (C-7):

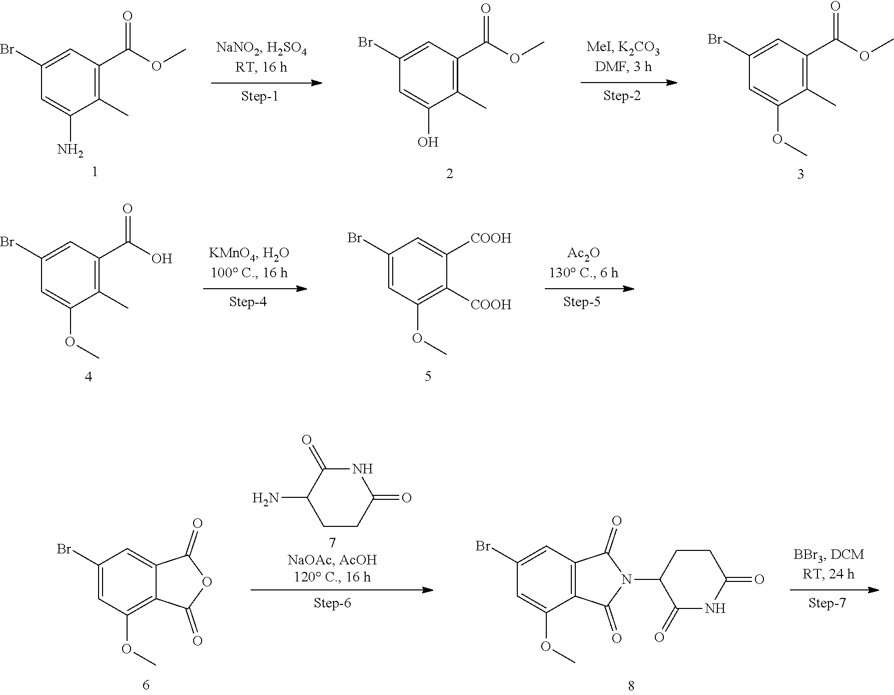

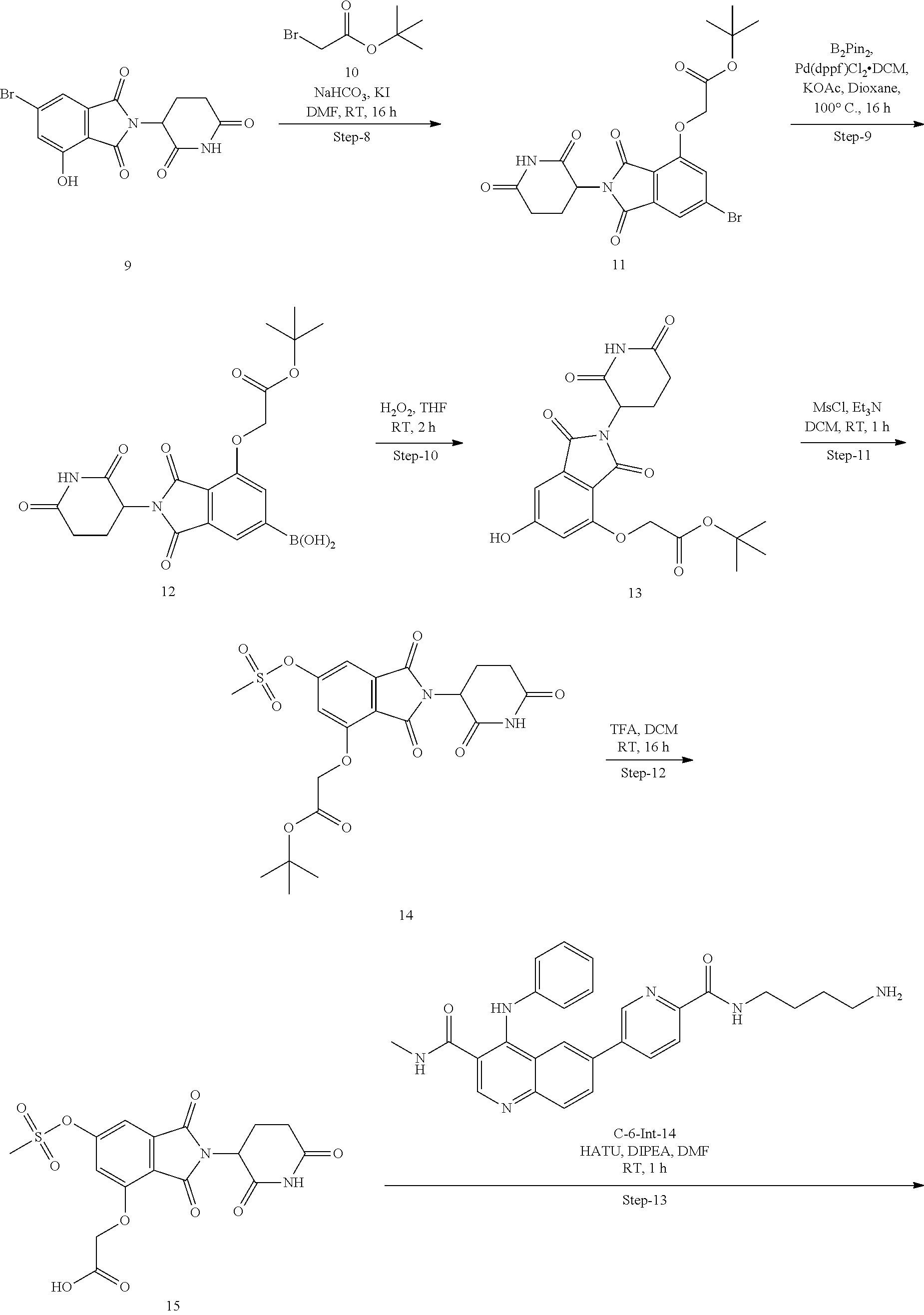

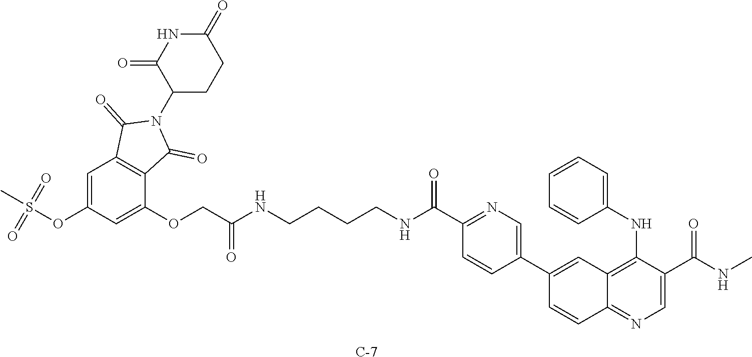

To a stirred solution of methyl 3-amino-5-bromo-2-methyl-benzoate 1 (9 g, 36.87 mmol) in conc. sulfuric acid (15 mL) was added NaNO2 (3.06 g, 44.35 mmol) in portions at 5 0° C. The resulting mixture was stirred at room temperature for 16 hours. After completion, the resulting mixture was diluted with water (50 mL) and extracted with ethyl acetate (3×200 m). The combined organic layers were washed with saturated sodium chloride solution (100 mL) and dried over sodium sulfate, concentrated under reduced pressure to afford crude product, which was purified by flash column chromatography (Davisil silica) using 20% ethyl 10 acetate in petroleum ether to afford methyl 5-bromo-3-hydroxy-2-methyl-benzoate 2 (5.1 g, 34% yield) as a light-yellow solid. LCMS (ES+): m z 246.10 [M+H]+.

Step-2: To a solution of methyl 5-bromo-3-hydroxy-2-methyl-benzoate 2 (3.6 g, 14.69 mmol) in DMF (30 mL) were added potassium carbonate (6.09 g, 44.07 mmol) followed by methyl 15 iodide (1.83 mL, 29.38 mmol) at room temperature. The reaction mixture was stirred at room temperature for 3 hours. After completion of the reaction, the reaction mixture was diluted with water (50 mL) and extracted with ethyl acetate (50 mL×3). The combined organic layer was washed with saturated sodium chloride solution (50 mL), dried over sodium sulfate and concentrated under reduced pressure to afford methyl 5-bromo-3-methoxy-2-methyl-benzoate 3 (2.8 g, 61% yield) as a brown solid which was used directly in the next step without further purification. LCMS (ES+): m z 259.22 [M+H]+.

Step-3: To a stirred solution of methyl 5-bromo-3-methoxy-2-methyl-benzoate 3 (2.8 g, 10.81 mmol) in H20 (20 mL), THF (20 mL) was added LiGH (5.18 g, 216.14 mmol). The mixture 25 was stirred at room temperature for 36 hours. After completion of the reaction, reaction mixture was diluted with ethyl acetate (50 mL) and acidified with iN aqueous HCl to pH~4-5. The aqueous layer was extracted with ethyl acetate (2×50 mL). The combined organic extracts were washed with iN aqueous HCl, water (50 mL), and saturated sodium chloride solution (50 mL), dried sodium sulfate and concentrated under reduced pressure to afford 5-bromo-3-methoxy-2-methyl-benzoic acid 4 (2.45 g, 85% yield) as a white solid. LCMS (ES*): m z 245.04 [M+H]+.

Step-4: To a stirred solution of 5-bromo-3-methoxy-2-methyl-benzoic acid 4 (2.45 g, 10 mmol) in water (50 mL) was added potassium permanganate (7.90 g, 49.99 mmol), KOH (2.80 g, 50 mmol) at room temperature and heated to 100° C. for 16 hours. After completion of the reaction, the reaction mixture was cooled to room temperature, the reaction mixture was acidified by using solid NaHSO3 to ~pH 4-5 and extracted with ethyl acetate (3×100 mL) the combined organic layer was dried over anhydrous sodium sulfate, evaporated under reduced pressure to afford 5-bromo-3-methoxy-phthalic acid 5 (1.6 g, 50% yield) as an off-white solid. LCMS (ES-): m z 273.18 [M-H]-.

Step-5: To a stirred solution of 5-bromo-3-methoxy-phthalic acid 5 (1.6 g, 5.82 mmol) in acetic anhydride (20 mL) was stirred at 130° C. for 6 hours. After completion of reaction, the reaction mixture was concentrated under reduced pressure to afford 6-bromo-4-methoxy-isobenzofuran-1,3-dione 6 (1.6 g, 77% yield) as a colorless solid. The crude product was used to the next step without further purification. LCMS (ES+): m z 257.08 [M+H]+.

Step-6: To a stirred solution of 6-bromo-4-methoxy-isobenzofuran-1,3-dione 6 (1.6 g, 6.22 mmol), 3-aminopiperidine-2,6-dione 7 (1.13 g, 6.85 mmol, Hydrochloric acid) in acetic acid (18.84 mL) was added potassium acetate (1.83 g, 18.67 mmol) and stirred at 100° C. for 16 hours. The progress of the reaction was monitored by LCMS. After completion of reaction, the reaction mixture was concentrated under reduced pressure to get the crude product. The crude product was diluted with water (50 mL) to form the precipitate, the obtained precipitate was filtered and washed with water (30 mL) and dried under reduced pressure to afford 6-bromo-2-(2,6-dioxo-3-piperidyl)-4-methoxy-isoindoline-1,3-dione 8 (1.8 g, 79% yield) as a purple color solid. LCMS (ES+): m z 369.28 [M+H]+.

Step-7: To a stirred solution of 6-bromo-2-(2,6-dioxo-3-piperidyl)-4-methoxy-isoindoline-1,3-dione 8 (1.7 g, 4.63 mmol) in DCM (12 mL) was added boron tribromide (878 L, 9.26 mmol) dropwise at −78° C. The resulting mixture was stirred at room temperature for 16 hours. After completion, the resulting mixture was quenched with ice-cold water (20 mL) to form a precipitate. The obtained precipitate was filtered and dried under reduced pressure to afford 6-bromo-2-(2,6-dioxo-3-piperidyl)-4-hydroxy-isoindoline-1,3-dione 9 (1.4 g, 75% yield) as purple color solid. LCMS (ES*): m z 353.14 [M+H]+.

Step-8: To a solution of 6-bromo-2-(2,6-dioxo-3-piperidyl)-4-hydroxy-isoindoline-1,3-dione 9 5 (1 g, 2.83 mmol) in DMF (10 mL) were added NaHCO₃ (713 mg, 8.50 mmol), potassium iodide (235 mg, 1.42 mmol) followed by tert-butyl 2-bromoacetate 10 (498 L, 3.40 mmol) at room temperature and stirred for 16 hours. After completion of the reaction, the reaction mixture was diluted with water (10 mL) to form a precipitate. The precipitate was filtered and dried under reduced pressure to afford tert-butyl 2-[6-bromo-2-(2,6-dioxo-3-piperidyl)-1,3-dioxo-isoindolin-4-yl]oxyacetate 11 (0.7 g, 47% yield) as a purple color solid. LCMS (ES-): m z 466.98 [M-H]-.

Step-9: To a stirred solution of tert-butyl 2-[6-bromo-2-(2,6-dioxo-3-piperidyl)-1,3-dioxo-isoindolin-4-yl]oxyacetate 11 (0.450 g, 0.963 mmol), 4,4,4′,4′,5,5,5′,5′-octamethyl-2,2′-bi-1,3,2-dioxaborolane (367 mg, 1.44 mmol) in 1,4-dioxane (10 mL). The mixture was degassed with nitrogen for 5 minutes, then were added Pd(dppf)C12 DCM (39 mg, 0.048 mmol), potassium acetate (283 mg, 2.89 mmol) and again degassed with nitrogen for 5 minutes. The reaction mixture was heated and stirred at 100° C. for 16 hours. After completion, the reaction mixture was concentrated under reduced pressure to afford [7-(2-tert-butoxy-2-oxo-ethoxy)-2-(2,6-dioxo-3-piperidyl)-1,3-dioxo-isoindolin-5-yl]boronic acid 12 (0.5 g, 50% yield) as a yellow solid. LCMS (ES+): m z 377.13 [M-tBu+H]+.

Step-10: To a stirred solution of [7-(2-tert-butoxy-2-oxo-ethoxy)-2-(2,6-dioxo-3-piperidyl)-1,3-dioxo-isoindolin-5-yl]boronic acid 12 (420 mg, 0.972 mmol) in THF (10 mL) was added 35% 25 hydrogen peroxide in water (1.50 mL, 48.96 mmol) at 0° C. The reaction mixture was warm to room temperature and stirred for 2 hours. After completion, the reaction mixture was quenched with saturated. sodium bisulfate solution (20 mL) and extracted with ethyl acetate (2×50 mL).

The combined organic layer was dried over sodium sulfate, filtered and concentrated under reduced pressure to afford tert-butyl 2-[2-(2,6-dioxo-3-piperidyl)-6-hydroxy-1,3-dioxo-isoindolin-4-yl]oxyacetate 13 (0.4 g, 72% yield) as a yellow solid. LCMS (ES-): m z 403.20 [M-H]-.

Step-11: To a stirred solution of tert-butyl 2-[2-(2,6-dioxo-3-piperidyl)-6-hydroxy-1,3-dioxo-isoindolin-4-yl]oxyacetate 13 (0.2 g, 0.494 mmol) in dichloromethane (4 mL) was added triethylamine (0.137 mL, 0.989 mmol) and stirred for 5 minutes. methane sulfonyl chloride (0.046 mL, 0.593 mmol) was added into the reaction mixture at 0° C. and stirred for 1 hour at room temperature. After completion of the reaction, the reaction mixture was diluted with saturated sodium bicarbonate solution (20 mL) and extracted with dichloromethane (2×20 mL). The combined organic layer was dried over anhydrous sodium sulfate and evaporated under reduced pressure to obtain the crude product tert-butyl 2-[2-(2,6-dioxo-3-piperidyl)-6-methylsulfonyloxy-1, 3-dioxo-isoindolin-4-yl]ox acetate 14 (0.18 g, 52% yield) as reddish-brown semi-solid, which was used directly in the next step without further purification. LC-MS (ES-): m z 481.04 [M-H]-.

Step-12: To a stirred solution of tert-butyl 2-[2-(2,6-dioxo-3-piperidyl)-6-methylsulfonyloxy-1, 3-dioxo-isoindolin-4-yl]ox acetate 14 (0.18 g, 0.373 mmol) in dichloromethane (3 mL) were added trifluoroacetic acid (0.342 mL, 4.48 mmol) at 0° C. The reaction mixture was stirred at room temperature for 16 hours. After completion of the reaction, the reaction mixture was concentrated under reduced pressure to obtain crude product. The crude product was triturated with diethyl ether (20 mL) to afford 2-[2-(2,6-dioxo-3-piperidyl)-6-methylsulfonyloxy-1, 3-dioxo-isoindolin-4-yl]ox acetic acid 15 (0.16 g, 58% yield) as a pale-brown solid. LC-MS (ES*): m z 427.10 [M+H]+.

Step-13: To a stirred solution of 6-[6-(4-aminobutylcarbamoyl)-3-pyridyl]-4-anilino-N-methyl-quinoline-3-carboxamide C6-Int-14 (80 mg, 0.137 mmol) and 2-[2-(2,6-dioxo-3-piperidyl)-6-methylsulfonyloxy-1,3-dioxo-isoindolin-4-yl]oxyacetic acid 15 (89 mg, 0.164 mmol) in dimethylformamide (2 mL) was added N,N-diisopropylethylamine (0.071 mL, 0.411 mmol) at room temperature and stirred for 5 min. HATU (78 mg, 0.205 mmol) was added into the reaction mixture and stirred for 1 hour at room temperature. After completion of the reaction, the reaction mixture was purified by MS Prep HPLC to afford [7-[2-[4-[[5-[4-anilino-3-(methylcarbamoyl)-6-quinolyl]pyridine-2-carbonyl]amino]butylamino]-2-oxo-ethoxy]-2-(2,6-dioxo-3-piperidyl)-1,3-dioxo-isoindolin-5-yl]methane sulfonate (C-7) (25.3 mg, 20% yield) as a light-yellow solid. LC-MS (ES+): m z 877.59 [M+H]+. ¹H-NMR (400 MHz, DMSO-d6): 6 11.12 (s, 1H), 10.06 (s, 1H), 8.82-8.74 (m, 3H), 8.52 (d, J=4.80 Hz, 1H), 8.35 (s, 1H), 8.19-8.16 (m, 2H), 8.09-8.05 (m, 2H), 8.02 (t, J=5.20 Hz, 1H), 7.47 (s, 1H), 7.41 (s, 1H), 7.32 (t, J=7.60 Hz, 2H), 7.10-7.05 (m, 3H), 5.16 (dd, J=5.20 Hz, 1H), 4.82 (s, 2H), 3.50 (s, 3H), 3.31 (s, 2H), 3.19-3.16 (m, 2H), 2.91-2.85 (m, 1H), 2.60-2.56 (m, 4H), 2.50-2.49 (m, 1H), 2.08-2.02 (m, 1H), 1.56-1.48 (m, 4H).

Prep-HPLC Method: X-BRIDGE C18 (19*250*5 um) Mobile phase A: 0.100 formic acid in water (Aq.) Mobile phase B: acetonitrile Gradient (Time!⁰oB): 0/10, 2/15, 10/35, 13/35, 13.1/100, 14/100, 14.1/10, 17/10. Flow rate: 18 mL/min Solubility: acetonitrile+tetrahydrofuran Non-limiting Examples of Compounds of the Present Invention

| | Experimental |
|---|---|
| | compound |
| Structure | number |
| 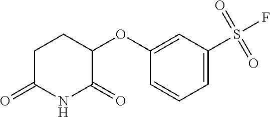 [SMILES: O=C1CCC(Oc2cccc(S(=O)(=O)F)c2)C(=O)N1] | A-1 |
| 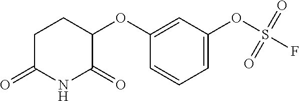 [SMILES: O=C1CCC(Oc2cccc(OS(=O)(=O)F)c2)C(=O)N1] | A-5 |
| 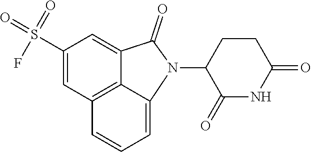 [SMILES: O=C1CCC(N2C(=O)c3cc(S(=O)(=O)F)cc4cccc2c34)C(=O)N1] | A-6 |
| 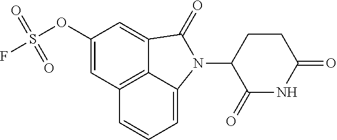 [SMILES: O=C1CCC(N2C(=O)c3cc(OS(=O)(=O)F)cc4cccc2c34)C(=O)N1] | A-7 |
| 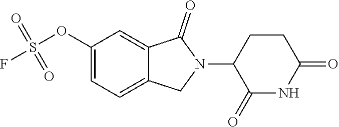 [SMILES: O=C1CCC(N2Cc3ccc(OS(=O)(=O)F)cc3C2=O)C(=O)N1] | A-8 |
| 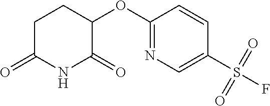 [SMILES: O=C1CCC(Oc2ccc(S(=O)(=O)F)cn2)C(=O)N1] | A-9 |
| 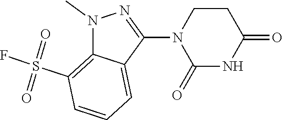 [SMILES: Cn1nc(N2CCC(=O)NC2=O)c2cccc(S(=O)(=O)F)c21] | A-10 |
| 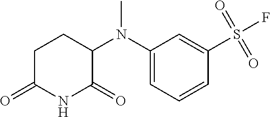 [SMILES: CN(c1cccc(S(=O)(=O)F)c1)C1CCC(=O)NC1=O] | A-11 |
| 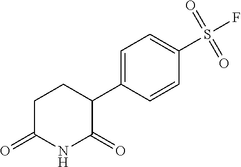 [SMILES: O=C1CCC(c2ccc(S(=O)(=O)F)cc2)C(=O)N1] | A-12 |
| 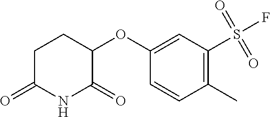 [SMILES: Cc1ccc(OC2CCC(=O)NC2=O)cc1S(=O)(=O)F] | A-13 |
| 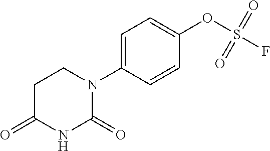 [SMILES: O=C1CCN(c2ccc(OS(=O)(=O)F)cc2)C(=O)N1] | A-15 |
| 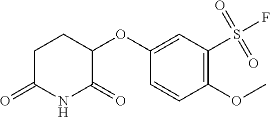 [SMILES: COc1ccc(OC2CCC(=O)NC2=O)cc1S(=O)(=O)F] | A-16 |
| 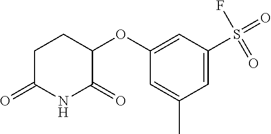 [SMILES: Cc1cc(OC2CCC(=O)NC2=O)cc(S(=O)(=O)F)c1] | A-17 |
| 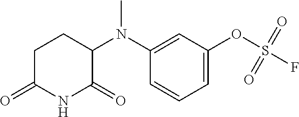 [SMILES: CN(c1cccc(OS(=O)(=O)F)c1)C1CCC(=O)NC1=O] | A-18 |
| 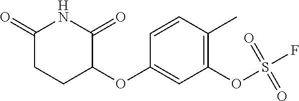 [SMILES: Cc1ccc(OC2CCC(=O)NC2=O)cc1OS(=O)(=O)F] | A-19 |
| 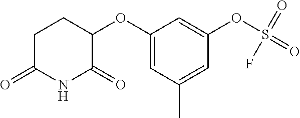 [SMILES: Cc1cc(OC2CCC(=O)NC2=O)cc(OS(=O)(=O)F)c1] | A-20 |
| 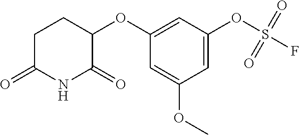 [SMILES: COc1cc(OC2CCC(=O)NC2=O)cc(OS(=O)(=O)F)c1] | A-21 |
| 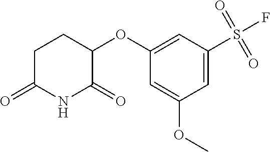 [SMILES: COc1cc(OC2CCC(=O)NC2=O)cc(S(=O)(=O)F)c1] | A-22 |
| 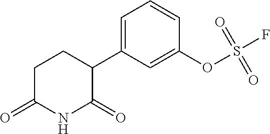 [SMILES: O=C1CCC(c2cccc(OS(=O)(=O)F)c2)C(=O)N1] | A-23 |
| 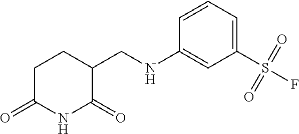 [SMILES: O=C1CCC(CNc2cccc(S(=O)(=O)F)c2)C(=O)N1] | A-24 |
| 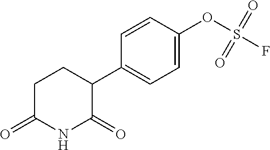 [SMILES: O=C1CCC(c2ccc(OS(=O)(=O)F)cc2)C(=O)N1] | A-25 |
| 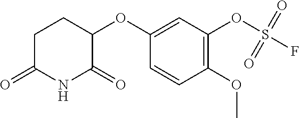 [SMILES: COc1ccc(OC2CCC(=O)NC2=O)cc1OS(=O)(=O)F] | A-27 |
| 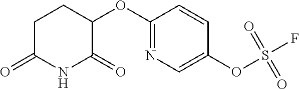 [SMILES: O=C1CCC(Oc2ccc(OS(=O)(=O)F)cn2)C(=O)N1] | A-28 |
| 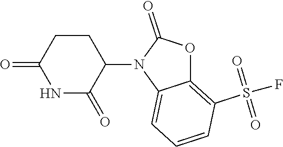 [SMILES: O=C1CCC(n2c(=O)oc3c(S(=O)(=O)F)cccc32)C(=O)N1] | A-29 |
| 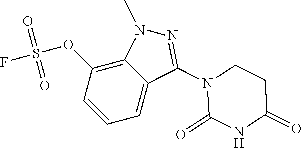 [SMILES: Cn1nc(N2CCC(=O)NC2=O)c2cccc(OS(=O)(=O)F)c21] | A-30 |
| 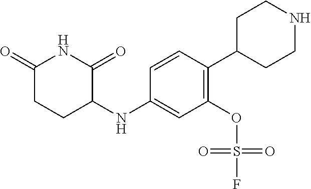 [SMILES: O=C1CCC(Nc2ccc(C3CCNCC3)c(OS(=O)(=O)F)c2)C(=O)N1] | A-31 |
| 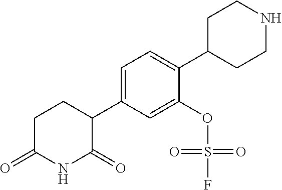 [SMILES: O=C1CCC(c2ccc(C3CCNCC3)c(OS(=O)(=O)F)c2)C(=O)N1] | A-32 |
| 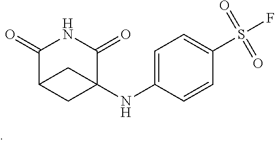 [SMILES: O=C1NC(=O)C2(Nc3ccc(S(=O)(=O)F)cc3)CC1C2] | A-33 |
| 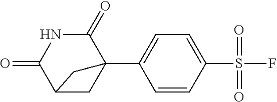 [SMILES: O=C1NC(=O)C2(c3ccc(S(=O)(=O)F)cc3)CC1C2] | A-34 |
| 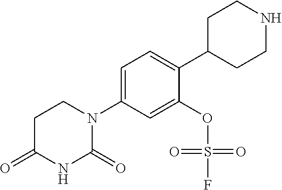 [SMILES: O=C1CCN(c2ccc(C3CCNCC3)c(OS(=O)(=O)F)c2)C(=O)N1] | A-35 |
| 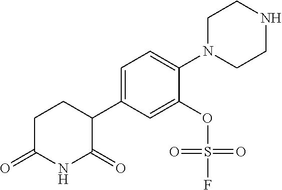 [SMILES: O=C1CCC(c2ccc(N3CCNCC3)c(OS(=O)(=O)F)c2)C(=O)N1] | A-36 |
| 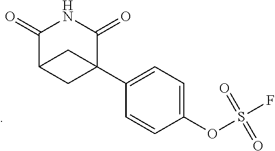 [SMILES: O=C1NC(=O)C2(c3ccc(OS(=O)(=O)F)cc3)CC1C2] | A-37 |
| 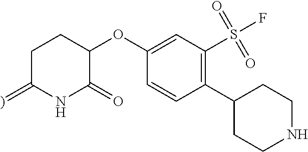 | A-38 |
| 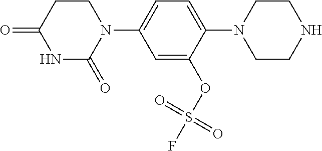 [SMILES: O=C1CCN(c2ccc(N3CCNCC3)c(OS(=O)(=O)F)c2)C(=O)N1] | A-39 |
| 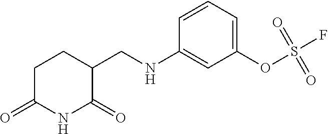 [SMILES: O=C1CCC(CNc2cccc(OS(=O)(=O)F)c2)C(=O)N1] | A-40 |
| 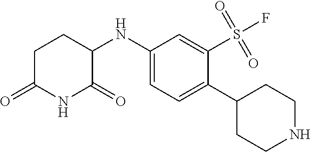 [SMILES: O=C1CCC(Nc2ccc(C3CCNCC3)c(S(=O)(=O)F)c2)C(=O)N1] | A-41 |
| 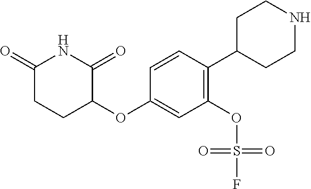 [SMILES: O=C1CCC(Oc2ccc(C3CCNCC3)c(OS(=O)(=O)F)c2)C(=O)N1] | A-42 |
| 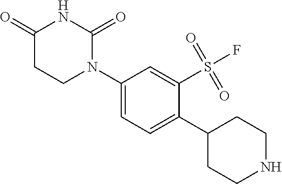 [SMILES: O=C1CCN(c2ccc(C3CCNCC3)c(S(=O)(=O)F)c2)C(=O)N1] | A-45 |
| 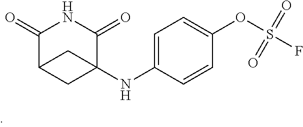 [SMILES: O=C1NC(=O)C2(Nc3ccc(OS(=O)(=O)F)cc3)CC1C2] | A-46 |
| 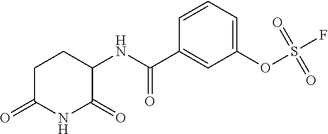 [SMILES: O=C1CCC(NC(=O)c2cccc(OS(=O)(=O)F)c2)C(=O)N1] | A-47 |
| 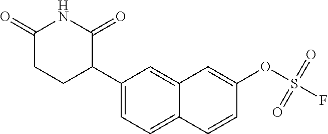 [SMILES: O=C1CCC(c2ccc3ccc(OS(=O)(=O)F)cc3c2)C(=O)N1] | A-48 |
| 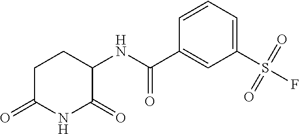 [SMILES: O=C1CCC(NC(=O)c2cccc(S(=O)(=O)F)c2)C(=O)N1] | A-49 |
| 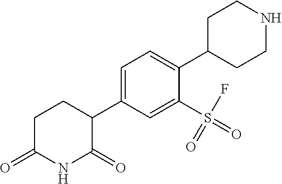 [SMILES: O=C1CCC(c2ccc(C3CCNCC3)c(S(=O)(=O)F)c2)C(=O)N1] | A-50 |
| 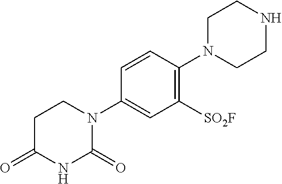 [SMILES: O=C1CCN(c2ccc(N3CCNCC3)c(S(=O)(=O)F)c2)C(=O)N1] | A-51 |
| 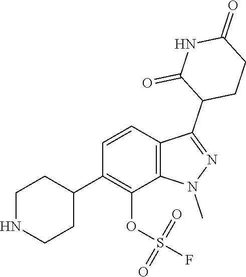 [SMILES: Cn1nc(C2CCC(=O)NC2=O)c2ccc(C3CCNCC3)c(OS(=O)(=O)F)c21] | A-52 |
| 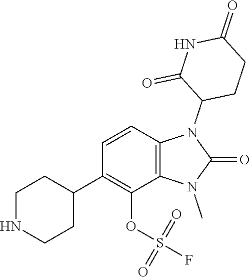 [SMILES: Cn1c(=O)n(C2CCC(=O)NC2=O)c2ccc(C3CCNCC3)c(OS(=O)(=O)F)c21] | A-53 |
| 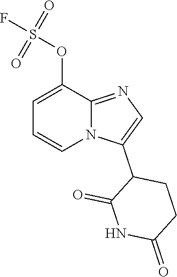 [SMILES: O=C1CCC(c2cnc3c(OS(=O)(=O)F)cccn23)C(=O)N1] | A-54 |
| 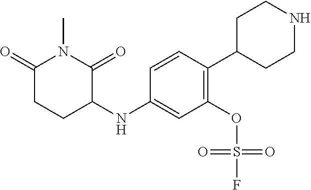 [SMILES: CN1C(=O)CCC(Nc2ccc(C3CCNCC3)c(OS(=O)(=O)F)c2)C1=O] | A-55 |
| 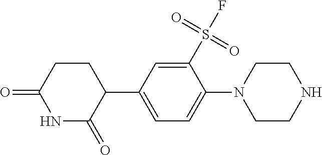 [SMILES: O=C1CCC(c2ccc(N3CCNCC3)c(S(=O)(=O)F)c2)C(=O)N1] | A-56 |
| 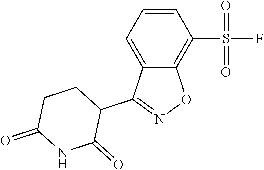 [SMILES: O=C1CCC(c2noc3c(S(=O)(=O)F)cccc23)C(=O)N1] | A-58 |
| 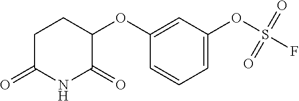 [SMILES: O=C1CCC(Oc2cccc(OS(=O)(=O)F)c2)C(=O)N1] | A-59 |
| 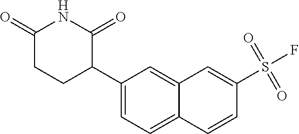 [SMILES: O=C1CCC(c2ccc3ccc(S(=O)(=O)F)cc3c2)C(=O)N1] | A-60 |
| 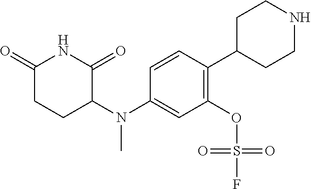 [SMILES: CN(c1ccc(C2CCNCC2)c(OS(=O)(=O)F)c1)C1CCC(=O)NC1=O] | A-61 |
| 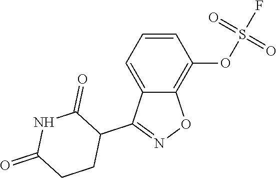 [SMILES: O=C1CCC(c2noc3c(OS(=O)(=O)F)cccc23)C(=O)N1] | A-62 |
| 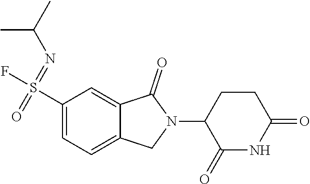 [SMILES: CC(C)N=S(=O)(F)c1ccc2c(c1)C(=O)N(C1CCC(=O)NC1=O)C2] | A-63 |
| 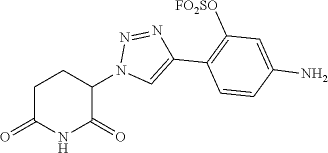 | A-67 |
| 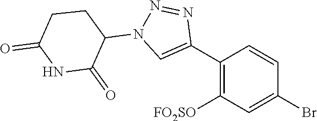 | A-68 |
| 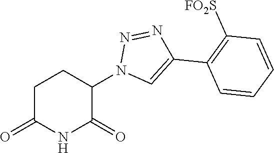 | A-69 |
| 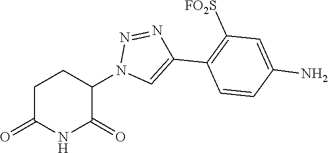 | A-70 |
| 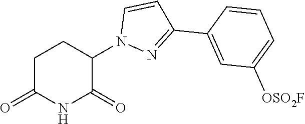 | A-71 |
| 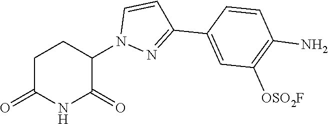 | A-72 |
| 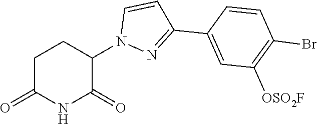 | A-73 |
| 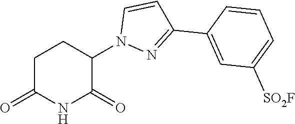 [SMILES: O=C1CCC(n2ccc(-c3cccc(S(=O)(=O)F)c3)n2)C(=O)N1] | A-74 |
| 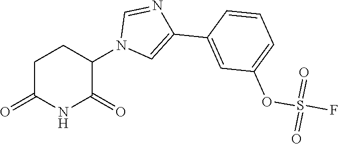 [SMILES: O=C1CCC(n2cnc(-c3cccc(OS(=O)(=O)F)c3)c2)C(=O)N1] | A-75 |
| 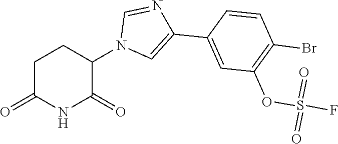 [SMILES: O=C1CCC(n2cnc(-c3ccc(Br)c(OS(=O)(=O)F)c3)c2)C(=O)N1] | A-76 |
| 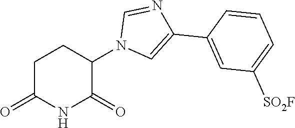 [SMILES: O=C1CCC(n2cnc(-c3cccc(S(=O)(=O)F)c3)c2)C(=O)N1] | A-77 |
| 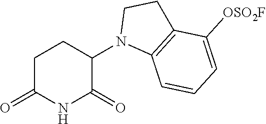 | A-78 |
| 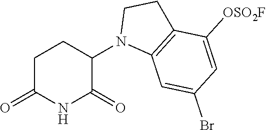 | A-79 |
| 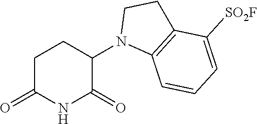 [SMILES: O=C1CCC(N2CCc3c2cccc3S(=O)(=O)F)C(=O)N1] | A-80 |
| 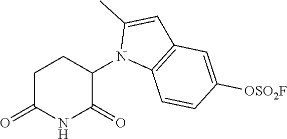 | A-81 |
| 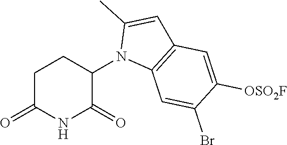 | A-82 |
| 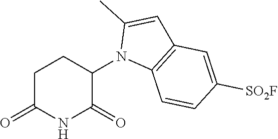 [SMILES: Cc1cc2cc(S(=O)(=O)F)ccc2n1C1CCC(=O)NC1=O] | A-83 |
| 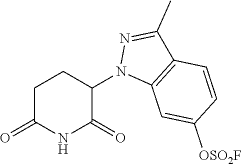 | A-84 |
| 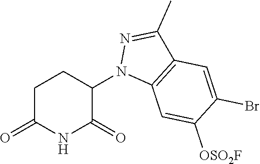 | A-85 |
| 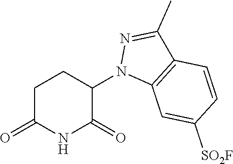 [SMILES: Cc1nn(C2CCC(=O)NC2=O)c2cc(S(=O)(=O)F)ccc12] | A-86 |
| 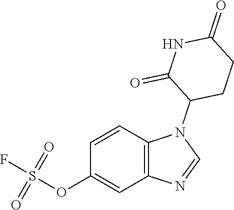 [SMILES: O=C1CCC(n2cnc3cc(OS(=O)(=O)F)ccc32)C(=O)N1] | A-87 |
| 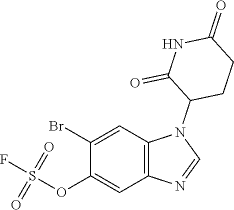 [SMILES: O=C1CCC(n2cnc3cc(OS(=O)(=O)F)c(Br)cc32)C(=O)N1] | A-88 |
| 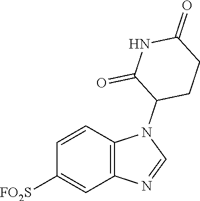 | A-89 |
| 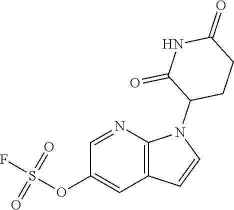 [SMILES: O=C1CCC(n2ccc3cc(OS(=O)(=O)F)cnc32)C(=O)N1] | A-90 |
| 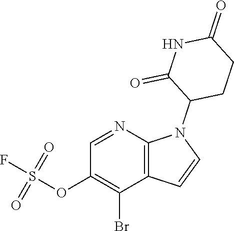 [SMILES: O=C1CCC(n2ccc3c(Br)c(OS(=O)(=O)F)cnc32)C(=O)N1] | A-91 |
| 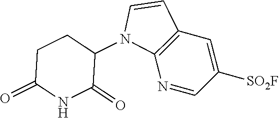 [SMILES: O=C1CCC(n2ccc3cc(S(=O)(=O)F)cnc32)C(=O)N1] | A-92 |
| 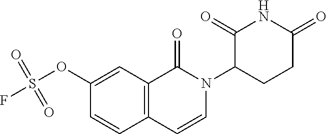 [SMILES: O=C1CCC(n2ccc3ccc(OS(=O)(=O)F)cc3c2=O)C(=O)N1] | A-93 |
| 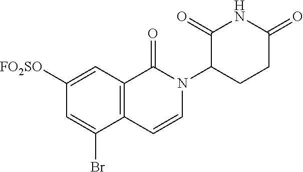 | A-94 |
| 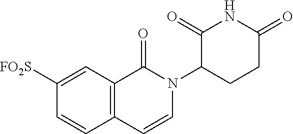 | A-95 |
| 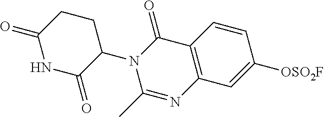 | A-96 |
| 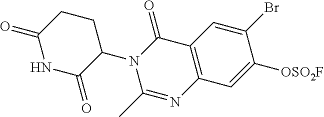 | A-97 |
| 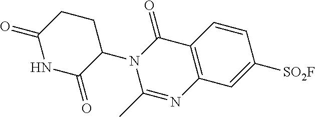 [SMILES: Cc1nc2cc(S(=O)(=O)F)ccc2c(=O)n1C1CCC(=O)NC1=O] | A-98 |
| 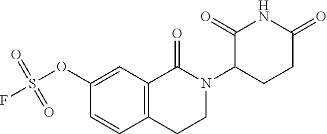 [SMILES: O=C1CCC(N2CCc3ccc(OS(=O)(=O)F)cc3C2=O)C(=O)N1] | A-99 |
| 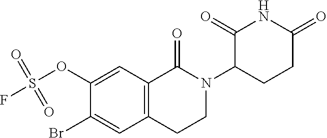 [SMILES: O=C1CCC(N2CCc3cc(Br)c(OS(=O)(=O)F)cc3C2=O)C(=O)N1] | A-100 |
| 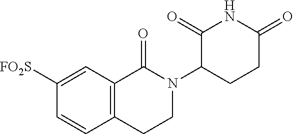 | A-101 |
| 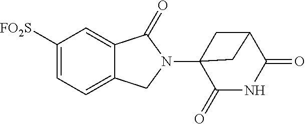 | A-102 |
| 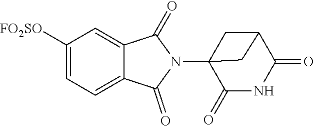 | A-103 |
| 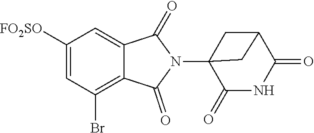 | A-104 |
| 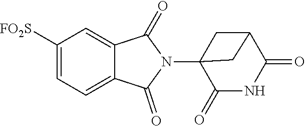 | A-105 |
| 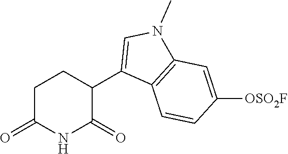 | A-106 |
| 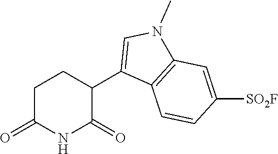 [SMILES: Cn1cc(C2CCC(=O)NC2=O)c2ccc(S(=O)(=O)F)cc21] | A-107 |
| 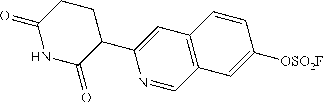 | A-108 |
| 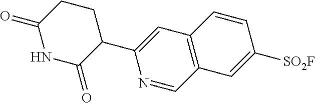 [SMILES: O=C1CCC(c2cc3ccc(S(=O)(=O)F)cc3cn2)C(=O)N1] | A-109 |
| 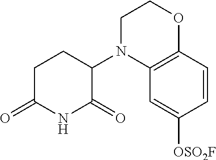 | A-110 |
| 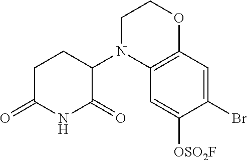 | A-111 |
| 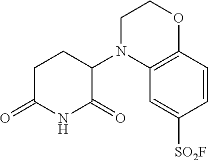 [SMILES: O=C1CCC(N2CCOc3ccc(S(=O)(=O)F)cc32)C(=O)N1] | A-112 |
| 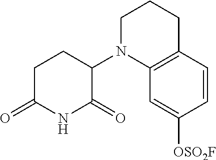 | A-113 |
| 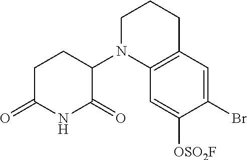 | A-114 |
| 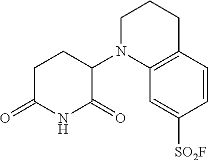 [SMILES: O=C1CCC(N2CCCc3ccc(S(=O)(=O)F)cc32)C(=O)N1] | A-115 |
| 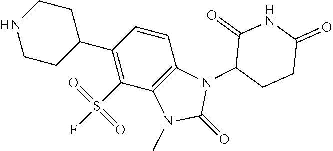 [SMILES: Cn1c(=O)n(C2CCC(=O)NC2=O)c2ccc(C3CCNCC3)c(S(=O)(=O)F)c21] | A-116 |
| 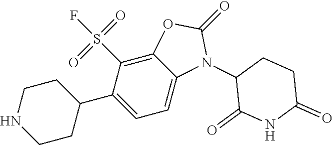 [SMILES: O=C1CCC(n2c(=O)oc3c(S(=O)(=O)F)c(C4CCNCC4)ccc32)C(=O)N1] | A-117 |
| 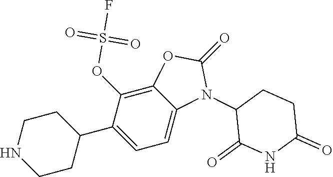 [SMILES: O=C1CCC(n2c(=O)oc3c(OS(=O)(=O)F)c(C4CCNCC4)ccc32)C(=O)N1] | A-118 |
| 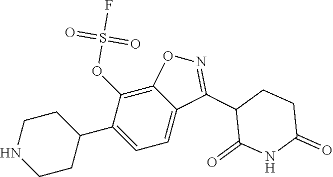 [SMILES: O=C1CCC(c2noc3c(OS(=O)(=O)F)c(C4CCNCC4)ccc23)C(=O)N1] | A-119 |
| 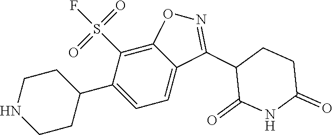 [SMILES: O=C1CCC(c2noc3c(S(=O)(=O)F)c(C4CCNCC4)ccc23)C(=O)N1] | A-120 |
| 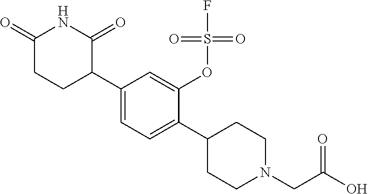 [SMILES: O=C(O)CN1CCC(c2ccc(C3CCC(=O)NC3=O)cc2OS(=O)(=O)F)CC1] | A-121 |
| 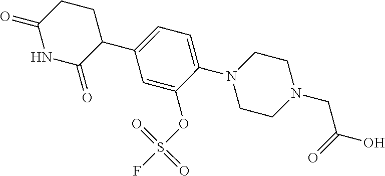 [SMILES: O=C(O)CN1CCN(c2ccc(C3CCC(=O)NC3=O)cc2OS(=O)(=O)F)CC1] | A-122 |
| 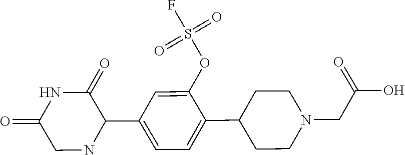 [SMILES: O=C(O)CN1CCC(c2ccc(C3NCC(=O)NC3=O)cc2OS(=O)(=O)F)CC1] | A-123 |
| 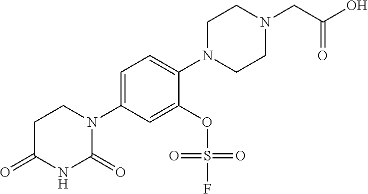 [SMILES: O=C(O)CN1CCN(c2ccc(N3CCC(=O)NC3=O)cc2OS(=O)(=O)F)CC1] | A-124 |
| 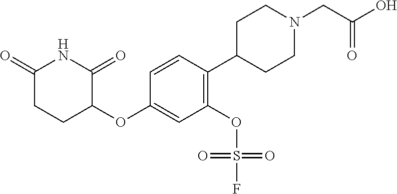 [SMILES: O=C(O)CN1CCC(c2ccc(OC3CCC(=O)NC3=O)cc2OS(=O)(=O)F)CC1] | A-125 |
| 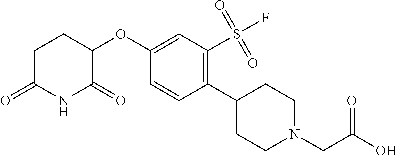 [SMILES: O=C(O)CN1CCC(c2ccc(OC3CCC(=O)NC3=O)cc2S(=O)(=O)F)CC1] | A-126 |
| 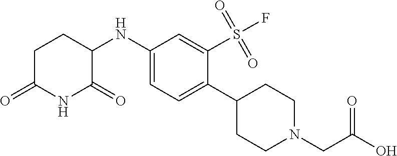 [SMILES: O=C(O)CN1CCC(c2ccc(NC3CCC(=O)NC3=O)cc2S(=O)(=O)F)CC1] | A-127 |
| 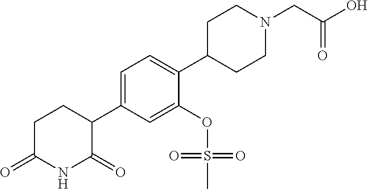 [SMILES: CS(=O)(=O)Oc1cc(C2CCC(=O)NC2=O)ccc1C1CCN(CC(=O)O)CC1] | A-128 |
| 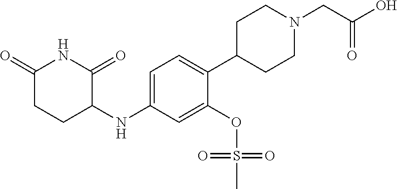 [SMILES: CS(=O)(=O)Oc1cc(NC2CCC(=O)NC2=O)ccc1C1CCN(CC(=O)O)CC1] | A-129 |
| 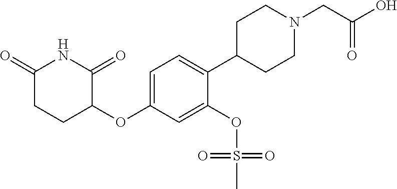 [SMILES: CS(=O)(=O)Oc1cc(OC2CCC(=O)NC2=O)ccc1C1CCN(CC(=O)O)CC1] | A-130 |
| 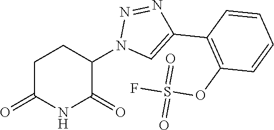 [SMILES: O=C1CCC(n2cc(-c3ccccc3OS(=O)(=O)F)nn2)C(=O)N1] | A-131 |
| 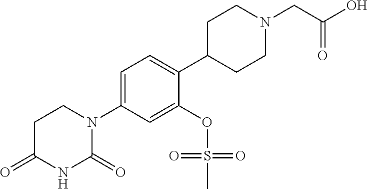 [SMILES: CS(=O)(=O)Oc1cc(N2CCC(=O)NC2=O)ccc1C1CCN(CC(=O)O)CC1] | A-132 |
| 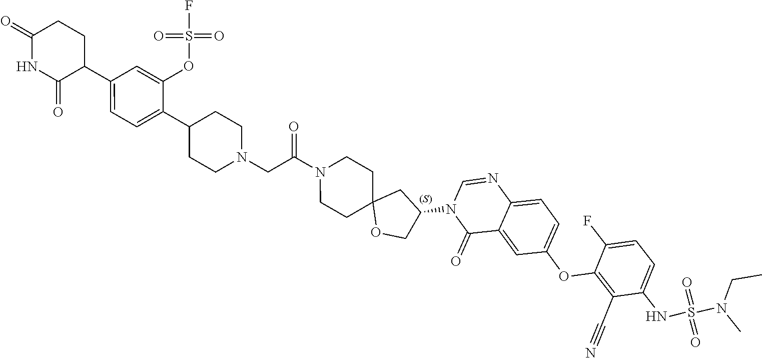 | B-1 |
| 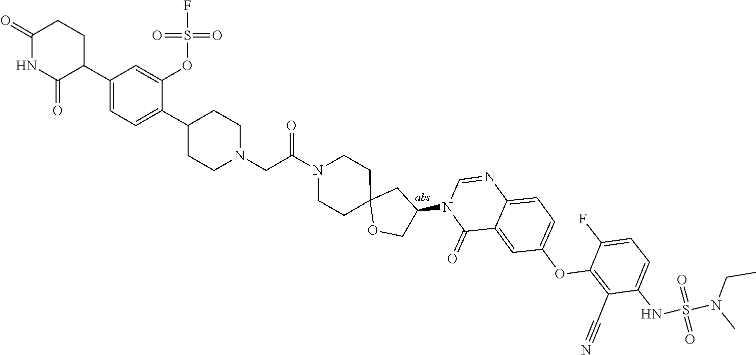 | B-2 |
| 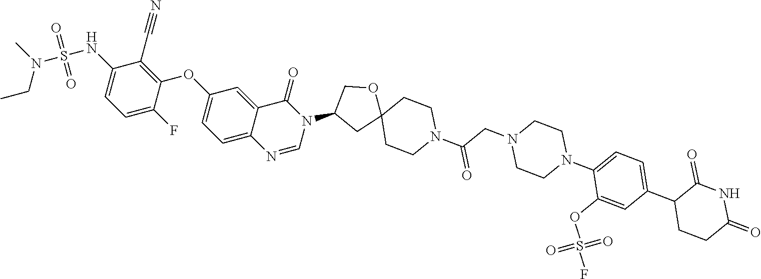 [SMILES: CCN(C)S(=O)(=O)Nc1ccc(F)c(Oc2ccc3ncn([C@H]4COC5(CCN(C(=O)CN6CCN(c7ccc(C8CCC(=O)NC8=O)cc7OS(=O)(=O)F)CC6)CC5)C4)c(=O)c3c2)c1C#N] | B-3 |
| 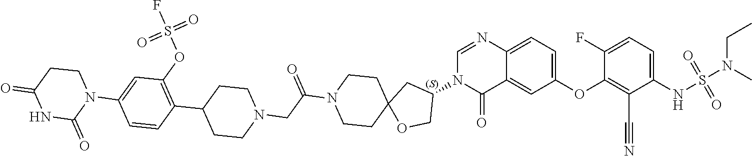 | B-4 |
| 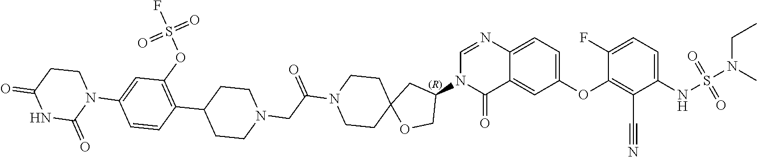 | B-5 |
| 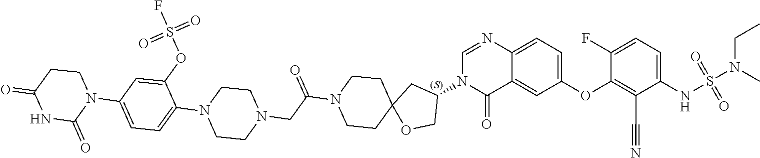 | B-6 |
| 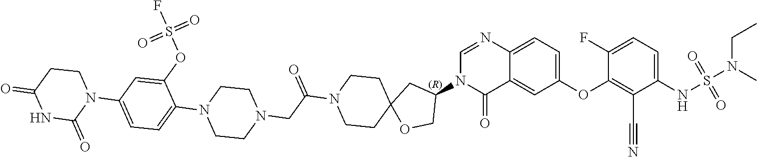 | B-7 |
| 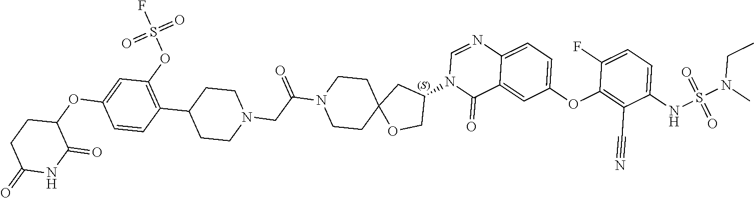 | B-8 |
| 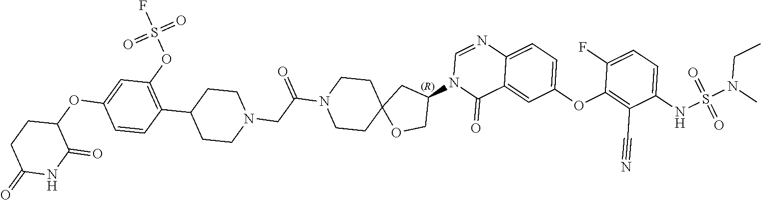 | B-9 |
| 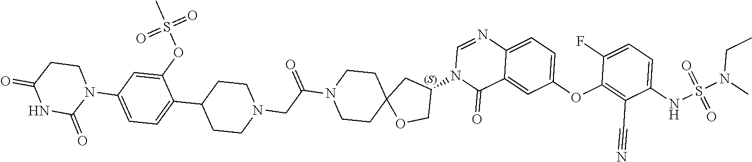 | B-10 |
| 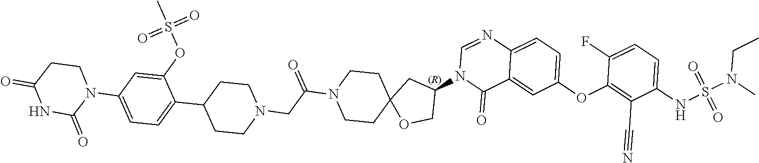 | B-11 |
| 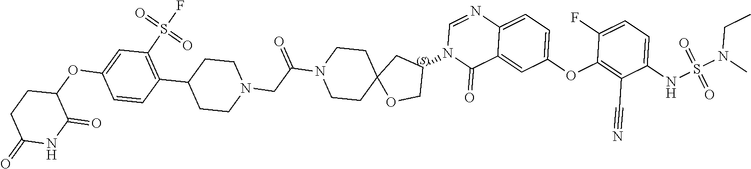 | B-12 |
| 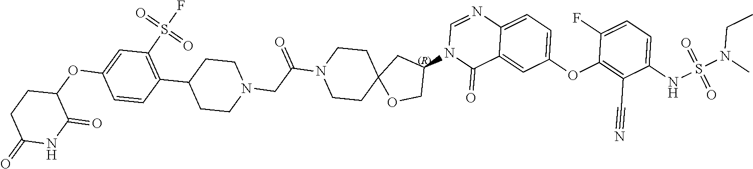 | B-13 |
| 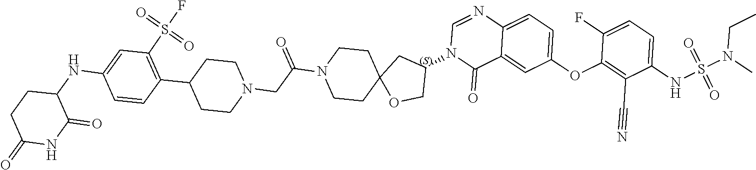 | B-14 |
| 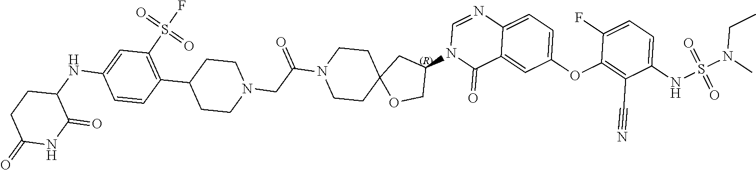 | B-15 |
| 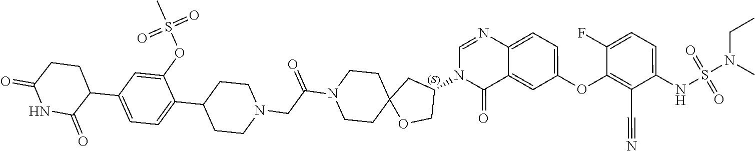 | B-16 |
| 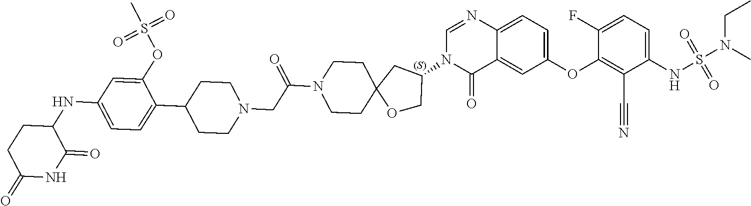 | B-17 |
| 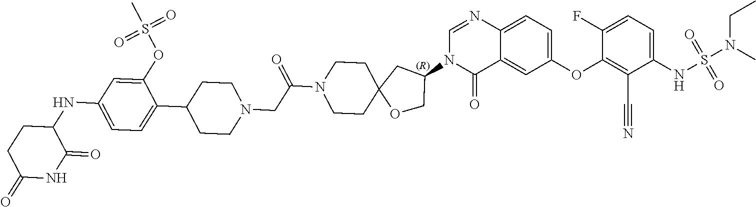 | B-18 |
| 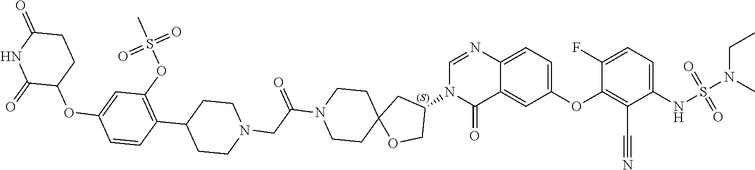 | B-19 |
| 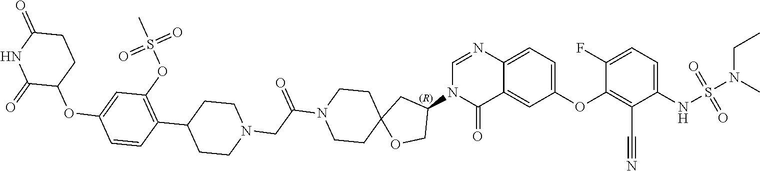 | B-20 |
| 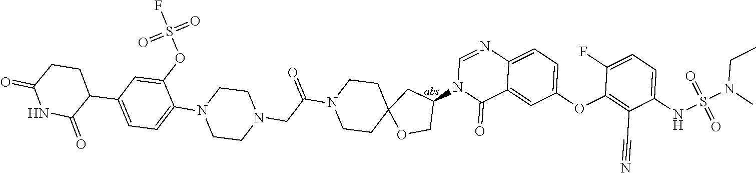 | B-21 |
| 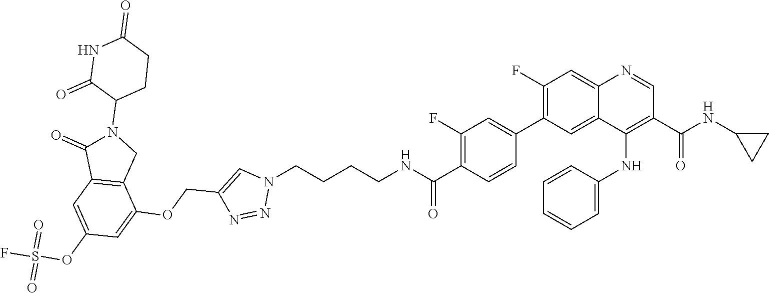 [SMILES: O=C1CCC(N2Cc3c(OCc4cn(CCCCNC(=O)c5ccc(-c6cc7c(Nc8ccccc8)c(C(=O)NC8CC8)cnc7cc6F)cc5F)nn4)cc(OS(=O)(=O)F)cc3C2=O)C(=O)N1] | C-2 |
| 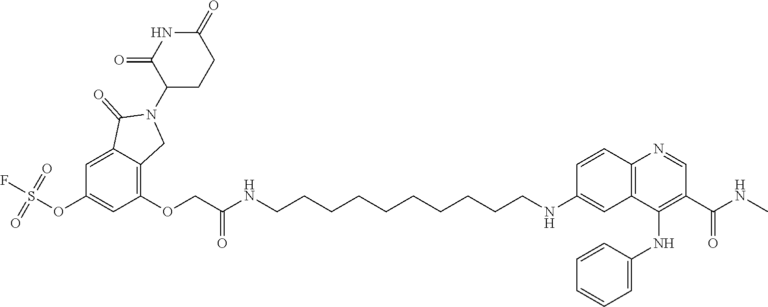 [SMILES: CNC(=O)c1cnc2ccc(NCCCCCCCCCCNC(=O)COc3cc(OS(=O)(=O)F)cc4c3CN(C3CCC(=O)NC3=O)C4=O)cc2c1Nc1ccccc1] | C-4 |
| 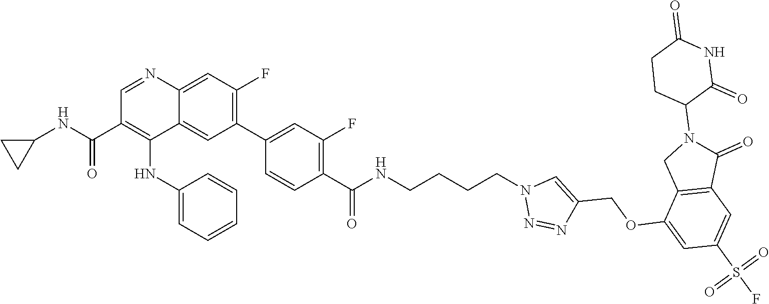 [SMILES: O=C1CCC(N2Cc3c(OCc4cn(CCCCNC(=O)c5ccc(-c6cc7c(Nc8ccccc8)c(C(=O)NC8CC8)cnc7cc6F)cc5F)nn4)cc(S(=O)(=O)F)cc3C2=O)C(=O)N1] | C-5 |
| 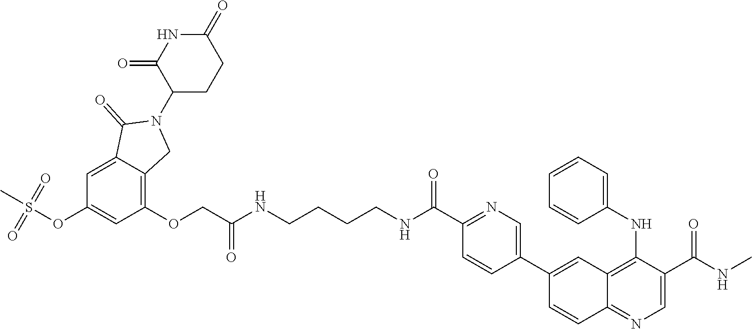 | C-6 |
| 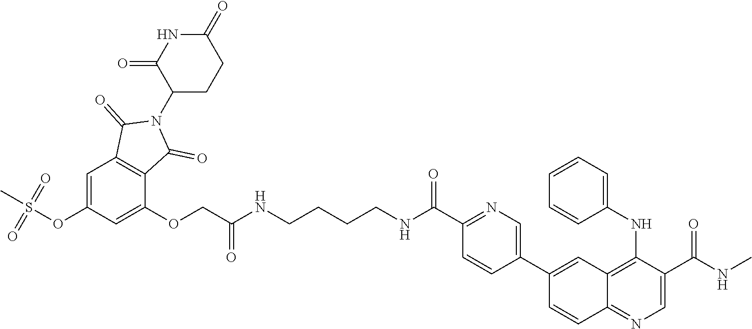 | C-7 |

### EXAMPLE 4: NanoBRET™ Target Engagement Assay Protocol

Selected compounds were tested for their binding affinity to cellular cereblon (CRBN) using the NanoBRET™ Target Engagement Assay (Promega, Madison, WI, USA). This assay uses bioluminescence resonance energy transfer (BRET) that creates a NanoBRET signal when NanoLuc (luciferase donor) tagged CRBN interacts with a NanoBRET fluorescent pomalidomide tracer. The CRBN binding affinity was determined by measuring the ability of test compounds to compete with the pomalidomide tracer to bind CRBN and therefore decrease the NanoBRET signal in a dose responsive manner.

Wild-type 293T cells were purchased from ATCC (Catalog No. CRL-3216, Manassas, VA, USA). To generate cells containing NanoLuc-CRBN, 293T cells were transduced with lentivirus containing a construct that induces exogenous expression of CRBN tagged with NanoLuc on the N-terminus (referred to here as cell line 293T.116). 293T.116 cells were maintained at 37° C. in an atmosphere of 5% CO2 in Dulbecco's modified Eagle Medium (DMEM) with high glucose (Catalog No. 11965092), supplemented with 10% fetal bovine serum (FBS; Catalog No. 10437036), and 10 mMHEPES (Catalog No. 15630130), all obtained from Thermofisher Scientific (Waltham, MA, USA).

Test compounds were prepared by dissolving neat compounds in DMSO (Catalog No. D8418, Sigma-Aldrich, Inc., St. Louis, MO, USA) to generate 10 mM stock solutions, which were serially diluted (half log) in DMSO to generate an 11-point dose series of 10000, 3160, 1000, 316, 100, 31.6, 10, 3.2, 1, 0.3, and 0.1 M (1000x final treatment concentration) in a 384-Well Low Dead Volume Microplate (Catalog No. LP-0200, Labcyte, San Jose, CA, USA).

Using the Echo 550 Acoustic Liquid Handler (Labcyte), 40 nL of serially diluted compound solutions were dispensed in duplicate to 384-well white TC-treated microplates (Catalog No. 3570, Corning, Glendale, CA, USA) and 40 nL of DMSO was dispensed to all control wells.

To the same 384-well plate, 40 nL of NanoBRET-pomalidomide tracer at a stock concentration of 300 uM was dispensed to all wells in columns 1-23 to be treated to cells at a final concentration of 300 nM, which was determined as the Kd for 293T.116. 40 nL of additional DMSO was dispensed to column 24.

To assess the binding affinity of compounds to cellular CRBN, 293T.116 cells were diluted to 200,000 cells/ml in phenol red free Opti-MEM media (Catalog No. 11058021, Thermofisher Scientific, Waltham, MA, USA) and plated at 40ul/well (8000 cells/well) to 384 well plates containing tracer and the 11-point dose series of compounds (final treatment concentration of highest dose was 10 μM). Plates were then spun briefly, and cells were incubated with compound and tracer at 37° C. for 2, 6, or 24 hours.

Following the incubation, NanoBRET TE Assay reagents were added as recommended by the manufacturer's instructions. Intracellular TE Nano-Glo Substrate/Inhibitor reagents were purchased from Promega (Catalog No. N2160, Madison, WI, USA). 3x Substrate/inhibitor solution was made up in Opti-MEM media with NanoBRET Nano-Glo Substrate at 1:166 dilution and Extracellular NanoLuc Inhibitor at 1:500 dilution.

Substrate/inhibitor solution was then added to all wells of the 384 well plates at 20 [l per well 5 and signal was acquired on the EnVision™ Multilabel Reader (PerkinElmer, Santa Clara, CA, USA). Donor emission from the CRBN-NanoLuc was detected with a 460/50 filter and the acceptor emission of the NanoBRET-pomalidomide tracer was detected with a 600 Long Pass filter.

Ratio of acceptor signal to donor signal was calculated for each well. Column 24 10 (DMSO only treatment) was designated the positive (P) control and column 23 (DMSO+Tracer treatment) was designated the negative (N) control. The percent response of compound treated samples was calculated by normalizing the Acceptor/Donor ratio (signal (T)) to the N controls from within the same 384 well plate after background signal subtraction (i.e., average P control).

$$Response \% = 100 \times ( Signal ( T ) - Average ( P ) ) / ( Average ( N ) - Average ( P ) )$$

Response % =100 x (Signal (T) - Average (P))/(Average (N) - Average (P))

Data were analyzed with a 4 parametric logistic fit to create dose response curves. The ICso value is the concentration of compound at which the fitted curves cross the 50% response level.

Table 1 NanoBRET TE 293T.116 CRBN 2 hours

TABLE 1
NanoBRET TE 293T.116 CRBN 2 hours

| | NanoBRET | NanoBRET | NanoBRET |
|---|---|---|---|
| | TE | TE | TE |
| | 293T.116 | 293T.116 | 293T.116 |
| | CRBN | CRBN | CRBN |
| | 2 hours | 2 hours | 2 hours |
| Compound | Emax | IC₅₀ nM | IP nM |
| 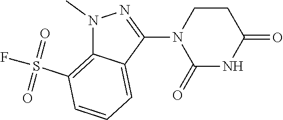 [SMILES: Cn1nc(N2CCC(=O)NC2=O)c2cccc(S(=O)(=O)F)c21] | *** | *** | *** |
| A-10 | | | |
| 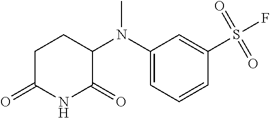 [SMILES: CN(c1cccc(S(=O)(=O)F)c1)C1CCC(=O)NC1=O] | *** | *** | *** |
| A-11 | | | |
| 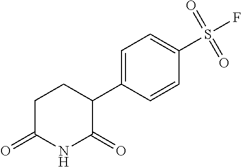 [SMILES: O=C1CCC(c2ccc(S(=O)(=O)F)cc2)C(=O)N1] | *** | *** | *** |
| A-12 | | | |
| 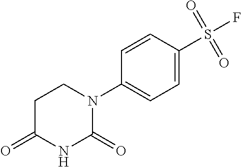 [SMILES: O=C1CCN(c2ccc(S(=O)(=O)F)cc2)C(=O)N1] | *** | ** | ** |
| A-14 | | | |
| 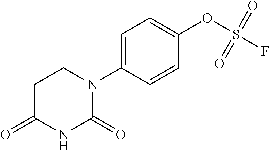 [SMILES: O=C1CCN(c2ccc(OS(=O)(=O)F)cc2)C(=O)N1] | ** | * | ** |
| A-15 | | | |
| 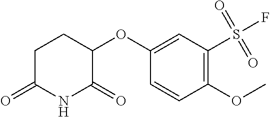 [SMILES: COc1ccc(OC2CCC(=O)NC2=O)cc1S(=O)(=O)F] | *** | *** | *** |
| A-16 | | | |
| 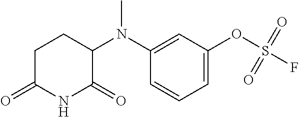 [SMILES: CN(c1cccc(OS(=O)(=O)F)c1)C1CCC(=O)NC1=O] | *** | ** | ** |
| A-18 | | | |
| 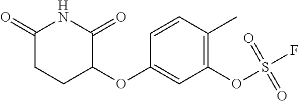 [SMILES: Cc1ccc(OC2CCC(=O)NC2=O)cc1OS(=O)(=O)F] | *** | ** | ** |
| A-19 | | | |
| 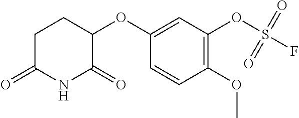 [SMILES: COc1ccc(OC2CCC(=O)NC2=O)cc1OS(=O)(=O)F] | *** | ** | ** |
| A-27 | | | |
| 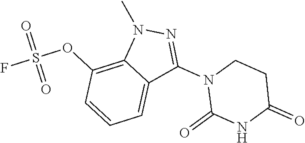 [SMILES: Cn1nc(N2CCC(=O)NC2=O)c2cccc(OS(=O)(=O)F)c21] | *** | ** | ** |
| A-30 | | | |
| 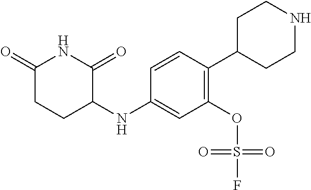 [SMILES: O=C1CCC(Nc2ccc(C3CCNCC3)c(OS(=O)(=O)F)c2)C(=O)N1] | *** | *** | *** |
| A-31 | | | |
| 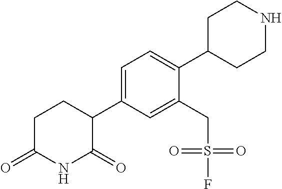 [SMILES: O=C1CCC(c2ccc(C3CCNCC3)c(CS(=O)(=O)F)c2)C(=O)N1] | *** | *** | *** |
| A-32 | | | |
| 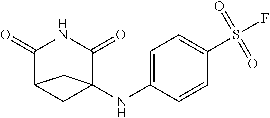 [SMILES: O=C1NC(=O)C2(Nc3ccc(S(=O)(=O)F)cc3)CC1C2] | *** | ** | ** |
| A-33 | | | |
| 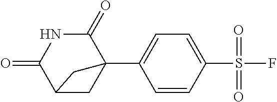 [SMILES: O=C1NC(=O)C2(c3ccc(S(=O)(=O)F)cc3)CC1C2] | *** | ** | ** |
| A-34 | | | |
| 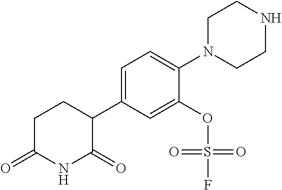 [SMILES: O=C1CCC(c2ccc(N3CCNCC3)c(OS(=O)(=O)F)c2)C(=O)N1] | *** | *** | *** |
| A-36 | | | |
| 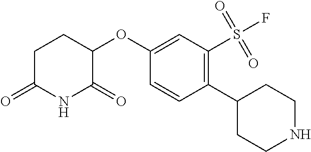 [SMILES: O=C1CCC(Oc2ccc(C3CCNCC3)c(S(=O)(=O)F)c2)C(=O)N1] | *** | *** | *** |
| A-38 | | | |
| 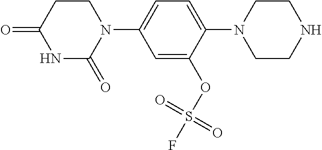 [SMILES: O=C1CCN(c2ccc(N3CCNCC3)c(OS(=O)(=O)F)c2)C(=O)N1] | ** | * | * |
| A-39 | | | |
| 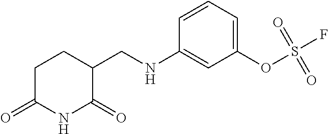 [SMILES: O=C1CCC(CNc2cccc(OS(=O)(=O)F)c2)C(=O)N1] | ** | * | * |
| A-40 | | | |

In the table above for Emax >=80% is *; <80% and >=50% is **; <50% is ***
In the table above for IC₅₀ and IP >=10 μM is *; <10 μM and >1 μM is **; <= 1 μM is ***

TABLE 2
NanoBRET TE 293T.116 CRBN 6 hours

| | NanoBRET TE | NanoBRET TE | NanoBRET TE |
|---|---|---|---|
| | 293T.116 | 293T.116 | 293T.116 |
| | CRBN | CRBN | CRBN |
| | 6 hours | 6 hours | 6 hours |
| Compound | Emax | IC₅₀ nM | IP nM |
| 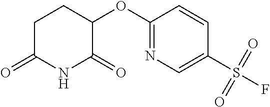 [SMILES: O=C1CCC(Oc2ccc(S(=O)(=O)F)cn2)C(=O)N1] | *** | ** | ** |
| A-9 | | | |
| 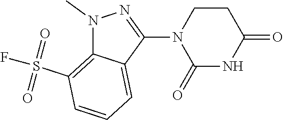 [SMILES: Cn1nc(N2CCC(=O)NC2=O)c2cccc(S(=O)(=O)F)c21] | *** | *** | *** |
| A-10 | | | |
| 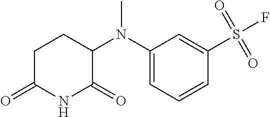 [SMILES: CN(c1cccc(S(=O)(=O)F)c1)C1CCC(=O)NC1=O] | *** | *** | *** |
| A-11 | | | |
| 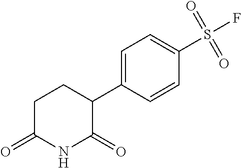 [SMILES: O=C1CCC(c2ccc(S(=O)(=O)F)cc2)C(=O)N1] | *** | *** | *** |
| A-12 | | | |
| 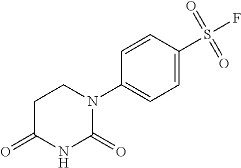 [SMILES: O=C1CCN(c2ccc(S(=O)(=O)F)cc2)C(=O)N1] | *** | ** | ** |
| A-14 | | | |
| 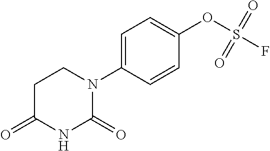 [SMILES: O=C1CCN(c2ccc(OS(=O)(=O)F)cc2)C(=O)N1] | ** | * | ** |
| A-15 | | | |
| 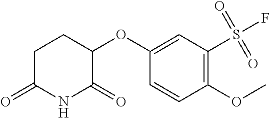 [SMILES: COc1ccc(OC2CCC(=O)NC2=O)cc1S(=O)(=O)F] | *** | *** | *** |
| A-16 | | | |
| 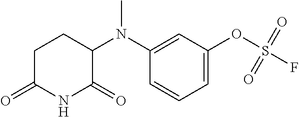 [SMILES: CN(c1cccc(OS(=O)(=O)F)c1)C1CCC(=O)NC1=O] | *** | ** | * |
| A-18 | | | |
| 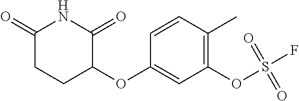 [SMILES: Cc1ccc(OC2CCC(=O)NC2=O)cc1OS(=O)(=O)F] | *** | ** | ** |
| A-19 | | | |
| 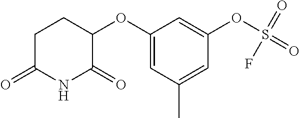 [SMILES: Cc1cc(OC2CCC(=O)NC2=O)cc(OS(=O)(=O)F)c1] | *** | *** | *** |
| A-20 | | | |
| 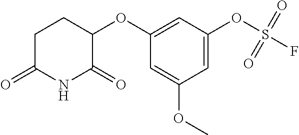 [SMILES: COc1cc(OC2CCC(=O)NC2=O)cc(OS(=O)(=O)F)c1] | *** | *** | *** |
| A-21 | | | |
| 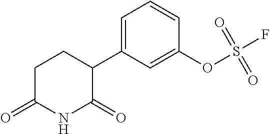 [SMILES: O=C1CCC(c2cccc(OS(=O)(=O)F)c2)C(=O)N1] | *** | ** | ** |
| A-23 | | | |
| 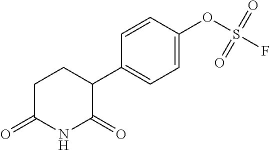 [SMILES: O=C1CCC(c2ccc(OS(=O)(=O)F)cc2)C(=O)N1] | *** | *** | *** |
| A-25 | | | |
| 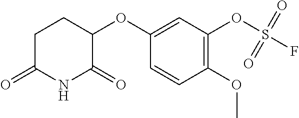 [SMILES: COc1ccc(OC2CCC(=O)NC2=O)cc1OS(=O)(=O)F] | *** | ** | ** |
| A-27 | | | |
| 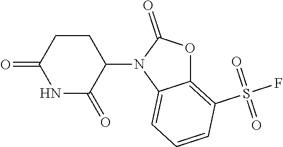 [SMILES: O=C1CCC(n2c(=O)oc3c(S(=O)(=O)F)cccc32)C(=O)N1] | *** | *** | *** |
| A-29 | | | |
| 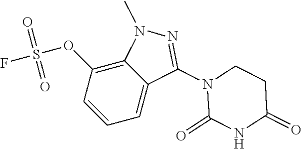 [SMILES: Cn1nc(N2CCC(=O)NC2=O)c2cccc(OS(=O)(=O)F)c21] | *** | *** | *** |
| A-30 | | | |
| 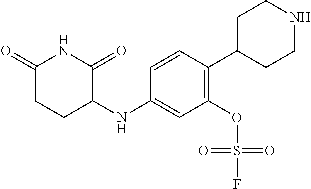 [SMILES: O=C1CCC(Nc2ccc(C3CCNCC3)c(OS(=O)(=O)F)c2)C(=O)N1] | *** | *** | *** |
| A-31 | | | |
| 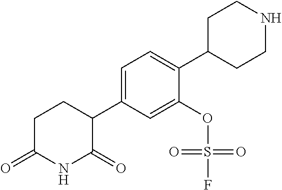 [SMILES: O=C1CCC(c2ccc(C3CCNCC3)c(OS(=O)(=O)F)c2)C(=O)N1] | *** | *** | *** |
| A-32 | | | |
| 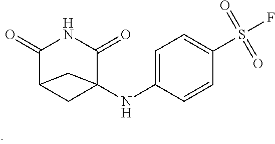 [SMILES: O=C1NC(=O)C2(Nc3ccc(S(=O)(=O)F)cc3)CC1C2] | *** | ** | ** |
| A-33 | | | |
| 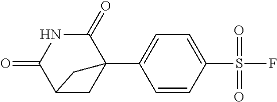 [SMILES: O=C1NC(=O)C2(c3ccc(S(=O)(=O)F)cc3)CC1C2] | *** | ** | ** |
| A-34 | | | |
| 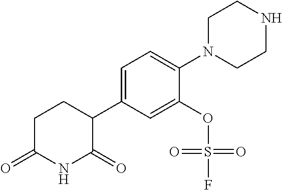 [SMILES: O=C1CCC(c2ccc(N3CCNCC3)c(OS(=O)(=O)F)c2)C(=O)N1] | *** | *** | *** |
| A-36 | | | |
| 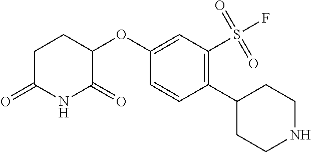 [SMILES: O=C1CCC(Oc2ccc(C3CCNCC3)c(S(=O)(=O)F)c2)C(=O)N1] | *** | *** | *** |
| A-38 | | | |
| 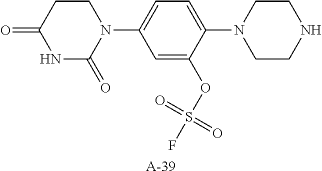 | *** | ** | * |
| 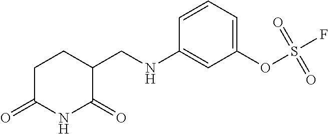 [SMILES: O=C1CCC(CNc2cccc(OS(=O)(=O)F)c2)C(=O)N1] | *** | ** | * |
| A-40 | | | |

In the table above for Emax >=80% is *; < 80% and >=50% is **; <50% is ***
In the table above for IC₅₀ and IP >=10 μM is *; <10 μM and >1 μM is **; <= 1 μM is ***

TABLE 3
NanoBRET TE 293T.116 CRBN 24 hours

| | NanoBRET TE | NanoBRET TE | NanoBRET TE |
|---|---|---|---|
| | 293T.116 | 293T.116 | 293T.116 |
| | CRBN | CRBN | CRBN |
| | 24 hours | 24 hours | 24 hours |
| Compound | Emax | IC₅₀ nM | IP nM |
| 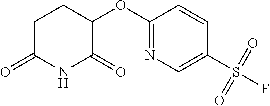 [SMILES: O=C1CCC(Oc2ccc(S(=O)(=O)F)cn2)C(=O)N1] | ** | * | * |
| A-9 | | | |
| 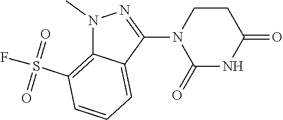 [SMILES: Cn1nc(N2CCC(=O)NC2=O)c2cccc(S(=O)(=O)F)c21] | *** | *** | *** |
| A-10 | | | |
| 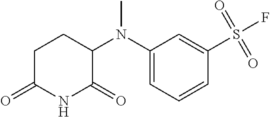 [SMILES: CN(c1cccc(S(=O)(=O)F)c1)C1CCC(=O)NC1=O] | *** | *** | *** |
| A-11 | | | |
| 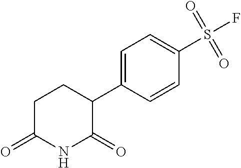 [SMILES: O=C1CCC(c2ccc(S(=O)(=O)F)cc2)C(=O)N1] | *** | ** | *** |
| A-12 | | | |
| 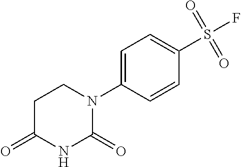 [SMILES: O=C1CCN(c2ccc(S(=O)(=O)F)cc2)C(=O)N1] | ** | * | ** |
| A-14 | | | |
| 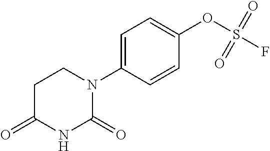 [SMILES: O=C1CCN(c2ccc(OS(=O)(=O)F)cc2)C(=O)N1] | *** | ** | ** |
| A-15 | | | |
| 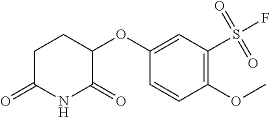 [SMILES: COc1ccc(OC2CCC(=O)NC2=O)cc1S(=O)(=O)F] | *** | *** | *** |
| A-16 | | | |
| 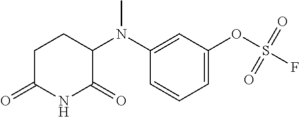 [SMILES: CN(c1cccc(OS(=O)(=O)F)c1)C1CCC(=O)NC1=O] | *** | ** | ** |
| A-18 | | | |
| 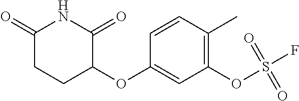 [SMILES: Cc1ccc(OC2CCC(=O)NC2=O)cc1OS(=O)(=O)F] | *** | ** | ** |
| A-19 | | | |
| 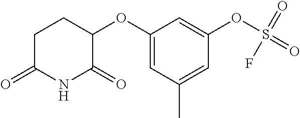 [SMILES: Cc1cc(OC2CCC(=O)NC2=O)cc(OS(=O)(=O)F)c1] | *** | ** | *** |
| A-20 | | | |
| 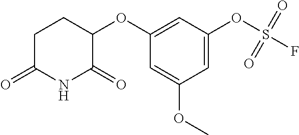 [SMILES: COc1cc(OC2CCC(=O)NC2=O)cc(OS(=O)(=O)F)c1] | *** | ** | ** |
| A-21 | | | |
| 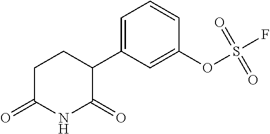 [SMILES: O=C1CCC(c2cccc(OS(=O)(=O)F)c2)C(=O)N1] | *** | ** | ** |
| A-23 | | | |
| 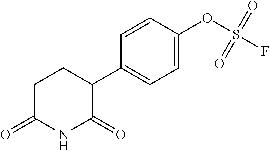 [SMILES: O=C1CCC(c2ccc(OS(=O)(=O)F)cc2)C(=O)N1] | *** | *** | *** |
| A-25 | | | |
| 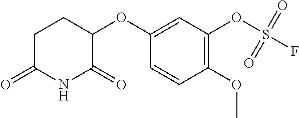 [SMILES: COc1ccc(OC2CCC(=O)NC2=O)cc1OS(=O)(=O)F] | ** | * | ** |
| A-27 | | | |
| 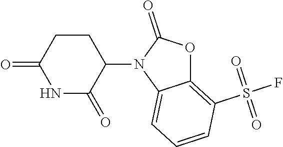 [SMILES: O=C1CCC(n2c(=O)oc3c(S(=O)(=O)F)cccc32)C(=O)N1] | *** | ** | ** |
| A-29 | | | |
| 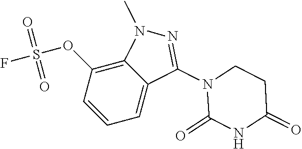 [SMILES: Cn1nc(N2CCC(=O)NC2=O)c2cccc(OS(=O)(=O)F)c21] | *** | ** | ** |
| A-30 | | | |
| 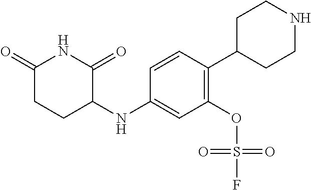 [SMILES: O=C1CCC(Nc2ccc(C3CCNCC3)c(OS(=O)(=O)F)c2)C(=O)N1] | *** | *** | *** |
| A-31 | | | |
| 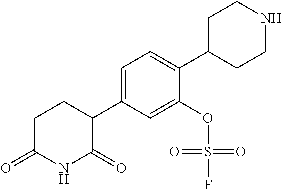 [SMILES: O=C1CCC(c2ccc(C3CCNCC3)c(OS(=O)(=O)F)c2)C(=O)N1] | *** | *** | *** |
| A-32 | | | |
| 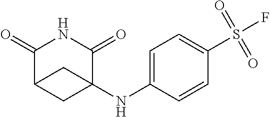 [SMILES: O=C1NC(=O)C2(Nc3ccc(S(=O)(=O)F)cc3)CC1C2] | *** | ** | ** |
| A-33 | | | |
| 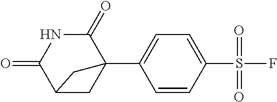 [SMILES: O=C1NC(=O)C2(c3ccc(S(=O)(=O)F)cc3)CC1C2] | * | * | * |
| A-34 | | | |
| 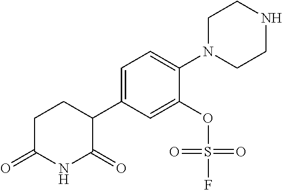 [SMILES: O=C1CCC(c2ccc(N3CCNCC3)c(OS(=O)(=O)F)c2)C(=O)N1] | *** | *** | *** |
| A-36 | | | |
| 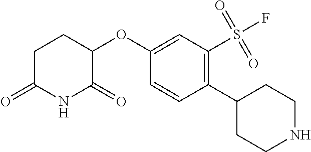 [SMILES: O=C1CCC(Oc2ccc(C3CCNCC3)c(S(=O)(=O)F)c2)C(=O)N1] | *** | *** | *** |
| A-38 | | | |
| 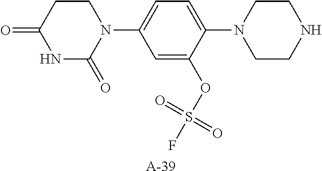 | *** | ** | ** |
| 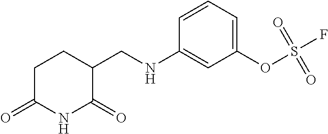 [SMILES: O=C1CCC(CNc2cccc(OS(=O)(=O)F)c2)C(=O)N1] | *** | ** | ** |
| A-40 | | | |

In the table above for Emax >=80% is *; <80% and >=50% is **; <50% is ***
In the table above for IC₅₀ and IP >=10 μM is *; <10 μM and >1 μM is **; <= 1 μM is ***

### EXAMPLE 5: CTGlo Cell Viability Assay

Compounds of the present invention were tested for their effect on the viability of A375—NRASQ^{61K} cells by quantification of ATP using the CellTiter-Glo® 2.0 Cell Viability Assay (Catalog No. G9243, Promega, Madison, WI, USA). This assay produces a luminescent signal in the presence of ATP, which is indicative of metabolically active cells, making the luminescence readout proportional to the number of viable cells. A375—NRASQ^{61K} cells, also referred to here as cell line A375.11, (harboring BRAF homozygous V600E mutation and NRAS homozygous Q61K mutation) were purchased from ATCC (Catalog No. CRL-1619IG-2, Manassas, VA, USA). A375—NRASQ⁶1K cells were maintained at 37° C. in an atmosphere of 5% CO2 in Dulbecco's modified Eagle Medium (DMEM) with high glucose (Catalog No. 11965092), supplemented with 10% fetal bovine serum (FBS; Catalog No. 10437036), and 10 mMHEPES (Catalog No. 15630130), all obtained from Thermofisher Scientific (Waltham, MA, USA). Cell culture flasks and 384-well microplates were acquired from VWR (Radnor, PA, USA).

Test compounds were prepared by dissolving neat compounds in DMSO (Catalog No. 10 D8418, Sigma-Aldrich, Inc., St. Louis, MO, USA) to generate 10 mM stock solutions, which were serially diluted (half log) in DMSO to generate an 11-point dose series (1000x final treatment concentration) in a 384-Well Low Dead Volume Microplate (Catalog No. LP-0200, Labcyte, San Jose, CA, USA). Using the Echo 550 Acoustic Liquid Handler (Labcyte), 50 nL of serially diluted compound solutions were dispensed in duplicate to 384-well TC-treated 15 microplates (Catalog No. 3571, Coming, Glendale, CA, USA) and 50 nL of DMSO was dispensed to control wells. A375—NRASQ61^{K} cells were resuspended in growth media and added to 384-well plates containing a 10-point dose series of compounds in duplicate (maximum 10 μM concentration for final treatment) at a density of 250 cells/well in 50 pl.

Cells were also plated to a 384-well plate with no compound treatment, to which CellTiter-Glo® reagent was added and luminescence was read right after plating to act as a baseline signal at time of plating (To). Plates treated with compound were then spun briefly, and cells were incubated with compound at 37° C. for 96 hours.

After 96 hours of compound treatment, CellTiter-Glo® reagent was added to the cells at 25 [l per well and luminescence was read out on the PHERAstar plate reader (BMG Labtech, Ortenberg, Germany). Cells treated with DMSO only (no test compound treatment) were used as the negative control (N). DMSO treated cells with no CellTiter-Glo® reagent added were used as the background or positive control (P). Viability was determined by subtracting out the average background signal (P) and normalized to the average untreated signal (N) set at 100%.

Data were analyzed with a 4-parameter logistic fit to generate sigmoidal dose-response curves. The half-maximal inhibition of cell growth (GI5o) is the concentration at which the fitted curve crosses 50%. The Emax, or maximum effect of each compound, represents the lowest % viability achieved following compound treatment.

TABLE 4
CTGlo A375.11 96 hours

| | CTGlo | CTGlo | CTGlo |
|---|---|---|---|
| | A375.11 | A375.119 | A375.11 |
| | 96 hours | 6 hours | 96 hours |
| Compound | Emax | GI₅₀ nM | IP nM |
| 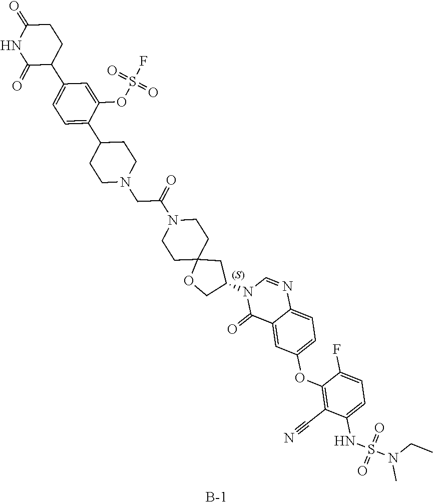 | ** | * | ** |
| 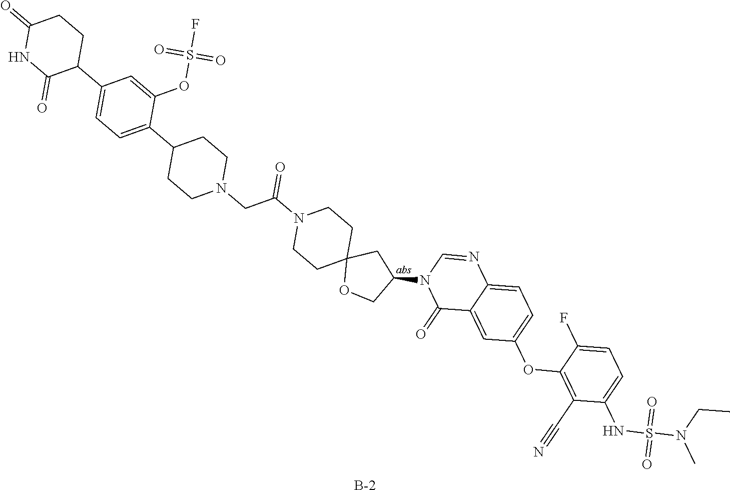 | *** | * | ** |
| 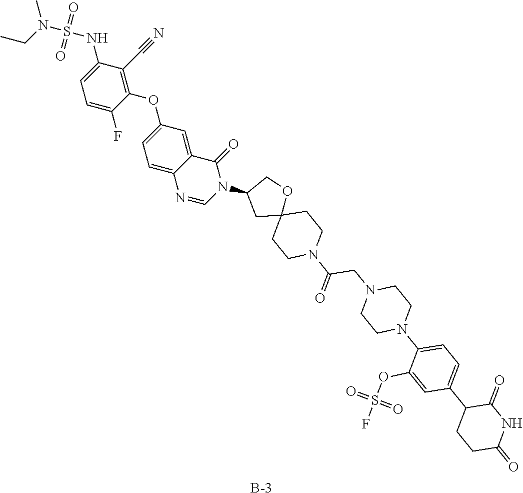 | ** | * | *** |
| 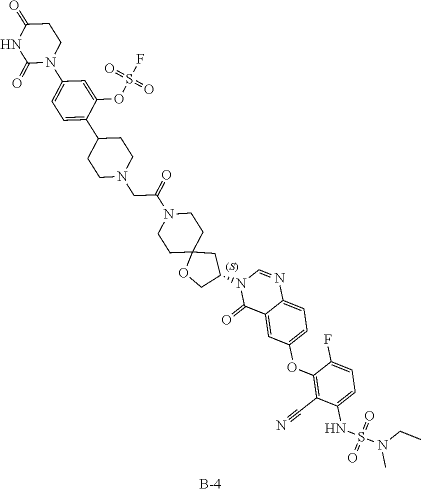 | ** | * | * |
| 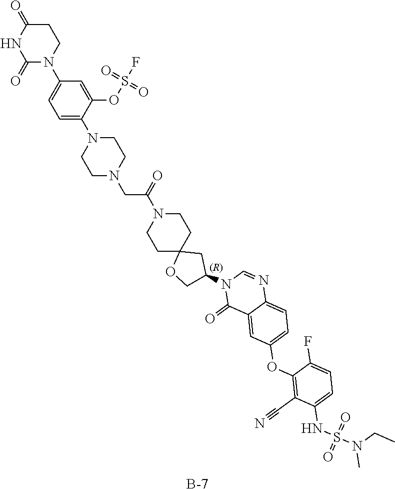 | ** | * | * |
| 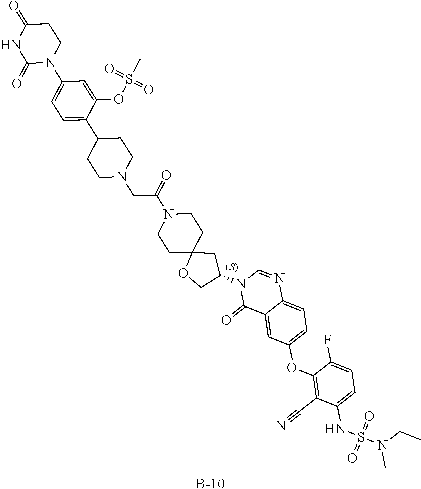 | ** | * | ** |
| 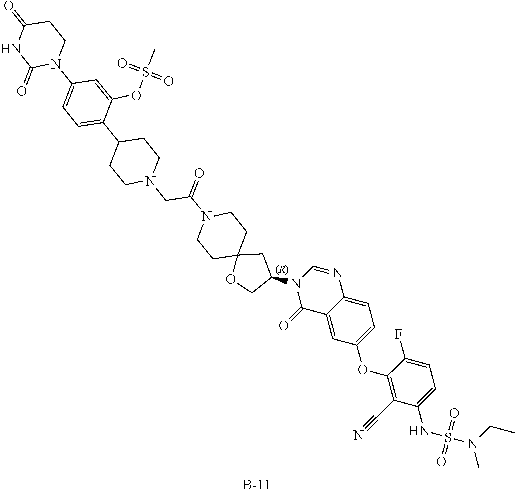 | *** | ** | ** |
| 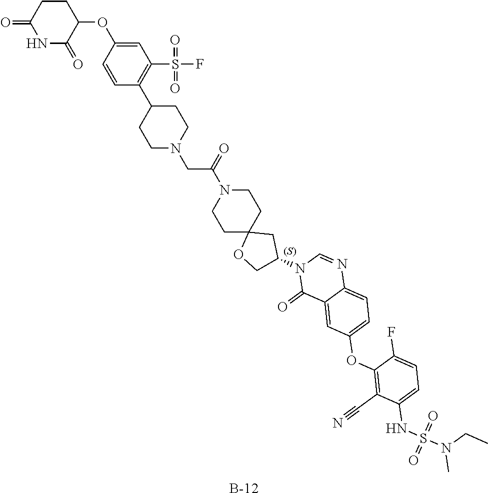 | *** | * | * |
| 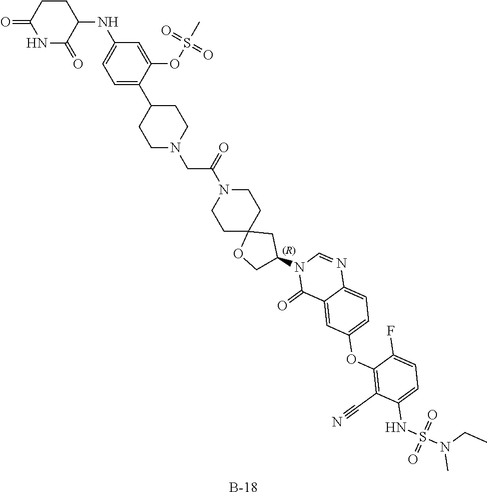 | *** | * | ** |
| 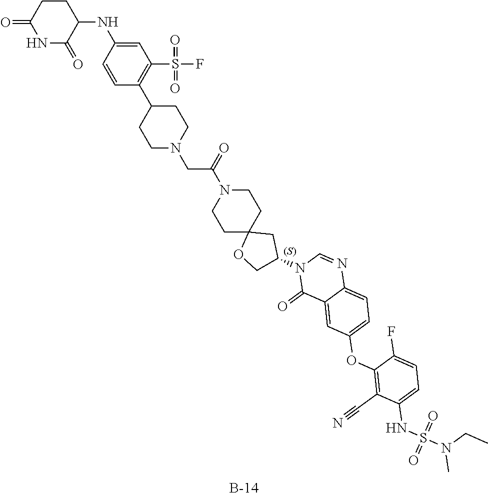 | ** | * | * |
| 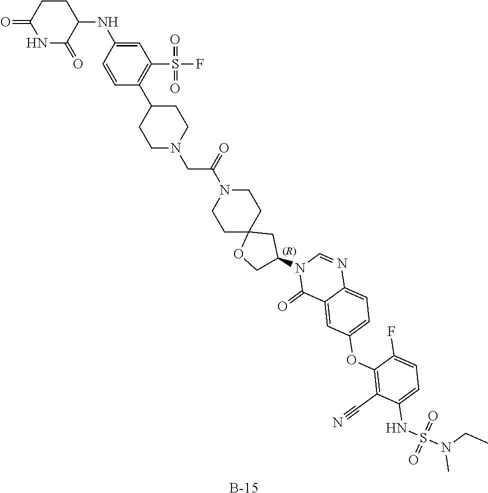 | ** | * | * |
| 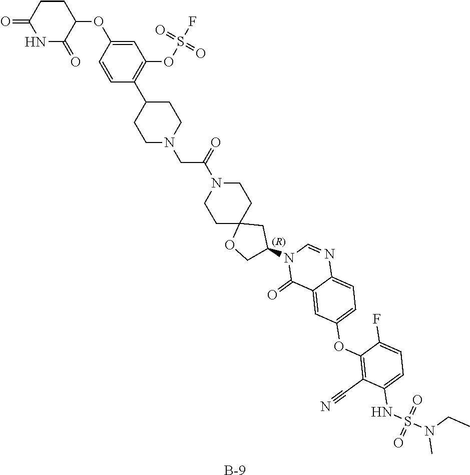 | ** | * | ** |
| 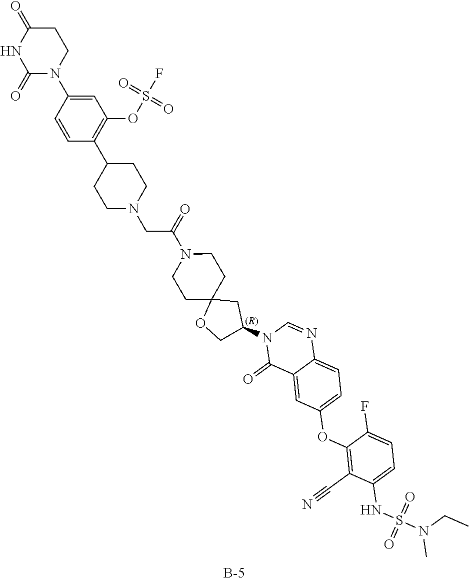 | ** | * | ** |
| 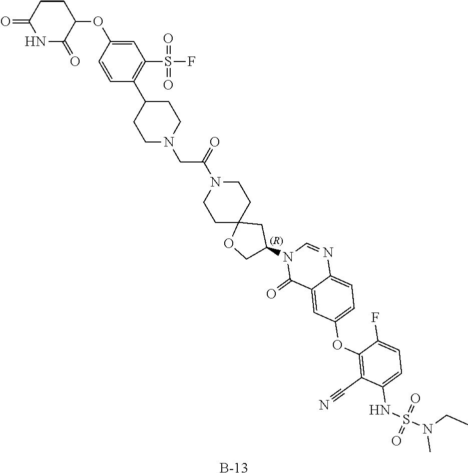 | ** | * | * |
| 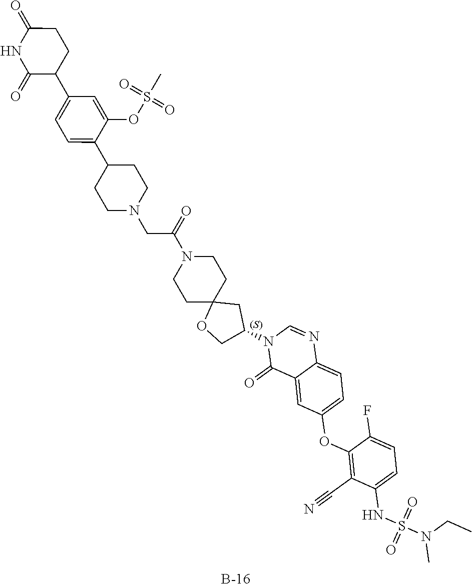 | ** | * | * |
| 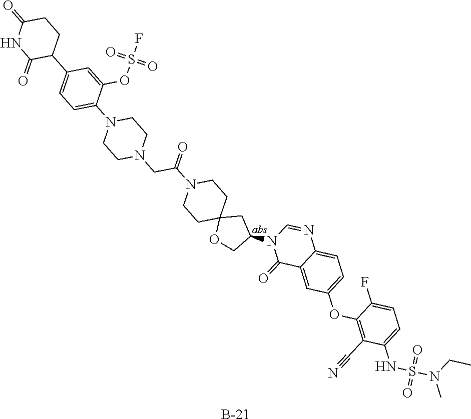 | ** | * | ** |
| 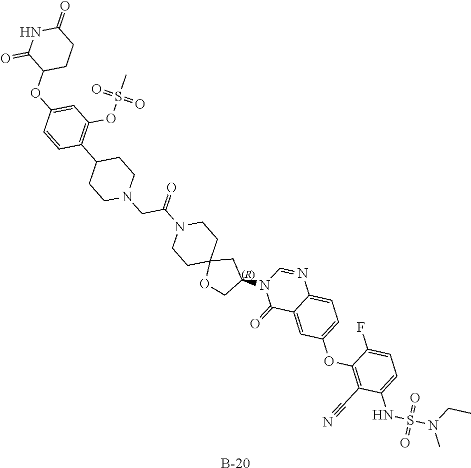 | ** | * | * |
| 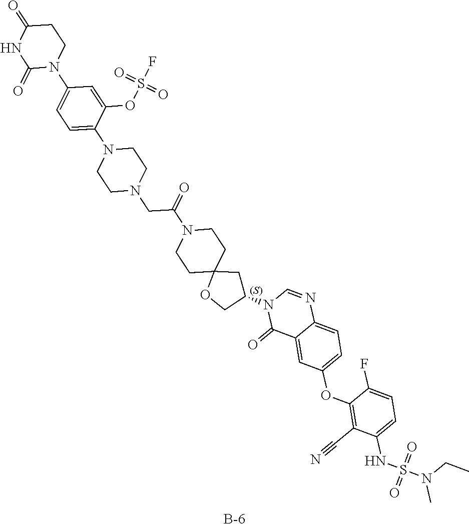 | ** | * | * |
| 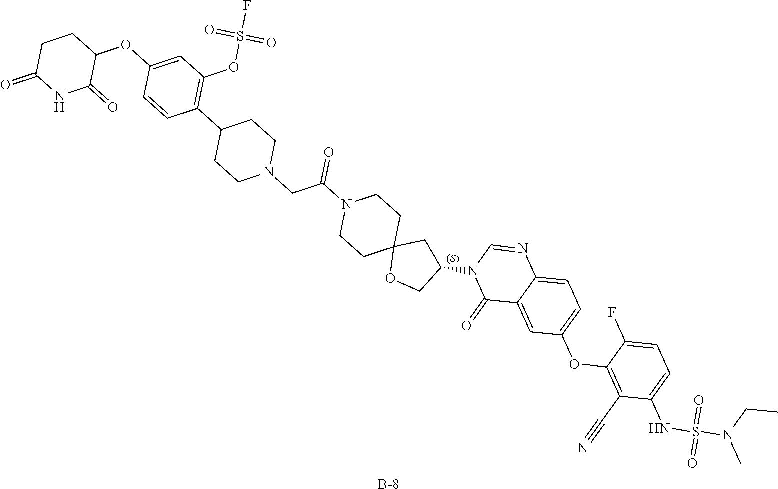 | ** | * | * |
| 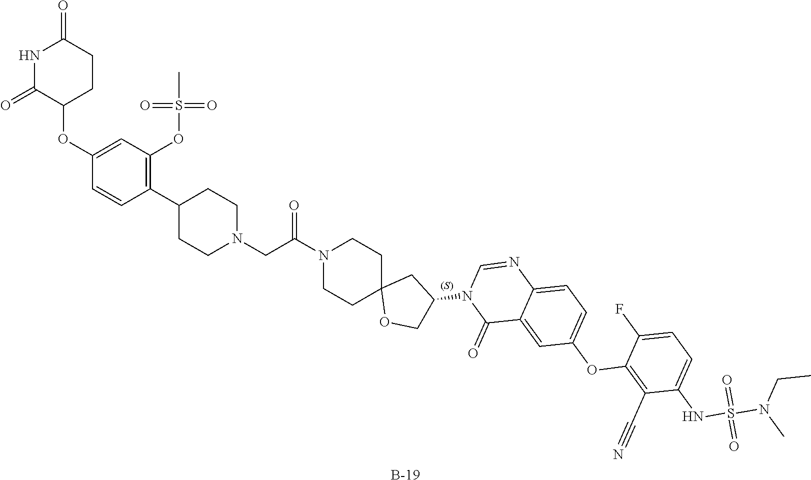 | ** | * | * |
| 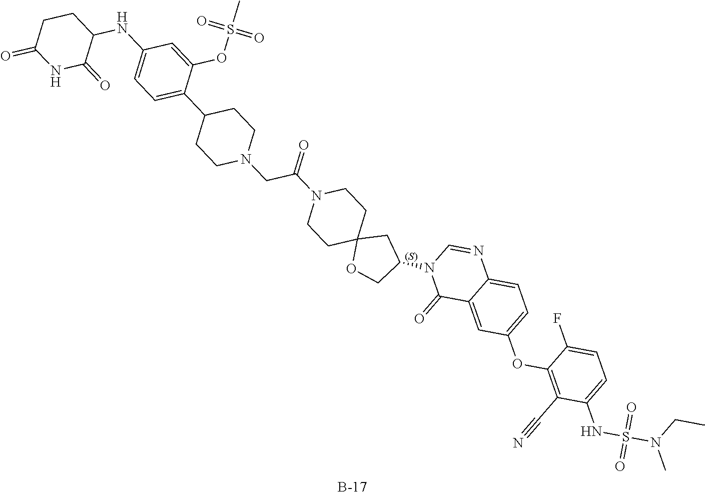 | * | * | * |

In the table above for Emax >=95% is *; <95% and >60% is **; <= 60% is ***
In the table above for GI₅₀ and IP >=10 μM is *; <10 μM and >1 μM is **; <= 1 μM is ***

### EXAMPLE 6: BRAF V600E HiBiT Degradation Assay

Compounds of this invention were tested for degradation of mutant BRAF V600E using the HiBiT assay, a luminescence-based assay for quantification of cellular protein levels using the Nano-Glo@HiBiT Lytic Detection System (Catalog No. N3050, Promega, Madison, WI USA). This assay utilizes the HiBiT tag on a protein of interest, which creates a luminescent signal in the presence of a complementing peptide and its substrate, the quantification of which 10 is proportional to the amount of HiBiT-tagged protein in the cell.

The cell line A375, which is homozygous for the BRAF V600E mutation, was purchased from ATCC (Catalog No. CRL-1619, Manassas, VA, USA). A375 cells were engineered to express HiBiT tagged BRAF V600E by CRISPR knock-in of the HiBiT tag to the endogenous BRAF V600E locus, followed by selection of a clonal population containing 15 the appropriate knock-in (referred to here as cell line A375.10). A375.10 cells were maintained at 37° C. in an atmosphere of 5% CO2 in Dulbecco's modified Eagle Medium (DMEM) with high glucose (Catalog No. 11965092), supplemented with 10% fetal bovine serum (FBS; Catalog No. 10437036), and 10 mM HEPES (Catalog No. 15630130), all obtained from Thermofisher Scientific (Waltham, MA, USA). Cell culture flasks and 384-well microplates 20 were acquired from VWR (Radnor, PA, USA).

Test compounds were prepared by dissolving neat compounds in DMSO (Catalog No.

D8418, Sigma-Aldrich, Inc., St. Louis, MO, USA) to generate 10 mM stock solutions, which were serially diluted (half log) in DMSO to generate an 11-point dose series (1000x final treatment concentration) in a 384-Well Low Dead Volume Microplate (Catalog No. LP-0200, 25 Labcyte, San Jose, CA, USA). Using the Echo 550 Acoustic Liquid Handler (Labcyte), 30 nL of serially diluted compound solutions were dispensed in duplicate to 384-well TC-treated microplates (Catalog No. 3571, Coming, Glendale, CA, USA) and 30 nL of DMSO was dispensed to control wells. A375.10 cells resuspended in growth media without phenol red (Catalog No. 21063-029, Thermofisher Scientific) were then added to 384-well plates 5 containing the 11-point dose series of compounds in duplicate (maximum 1OuM concentration for final treatment) at a density of 12,000 cells/well in 30 [l. Plates were then spun briefly, and cells were incubated with compound at 37° C. for 2 hoursor 24 hours.

Following the appropriate treatment time, Nano-Glo® HiBiT Lytic Assay reagents were added to the cells per the manufacturer's instructions by adding 30ul of reagent per well, and 10 the luminescence signal was acquired by plate reader. Cells treated with DMSO only (no test compound treatment) were used as the negative control (N). Media only (no cells added) with Nano-Glo reagent addition was used as the background or positive control (P). The percent response of compound treated samples were calculated by subtracting out the background signal (P) and normalizing to the DMSO control (N) as follows. % Response=100 x (Treated Signal - Average (P))/(Average (N) - Average (P)).

Data were analyzed with a 4-parameter logistic fit to generate sigmoidal dose-response curves. The DC5o is the concentration of compound at which 50% of the cellular BRAF V600E has been degraded. The Emax, or maximum effect of each compound, represents the amount of residual protein remaining in the cell following compound treatment. The IP, or inflection point of the dose-response curve, is the concentration of compound at which 50% of the observed degradation response is achieved.

The experiment was conducted at least once for each compound. If the experiment was conducted multiple times, the data shown in the table is the average of the two or more experiments.

TABLE 5
HiBiT-Degradation A375.10 BRAF 2 hours

| | HIBIT- | HiBIT- | HiBIT- |
|---|---|---|---|
| | Degradation | Degradation | Degradation |
| | A375.10 | A375.10 | A375.10 |
| | BRAF | BRAF | BRAF |
| | 2 hours | 2 hours | 2 hours |
| Compound | DC₅₀ nM | Emax | IP nM |
| 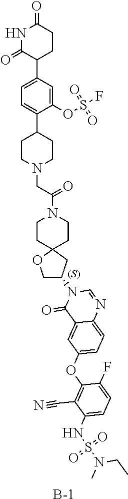 | ** | ** | *** |
| 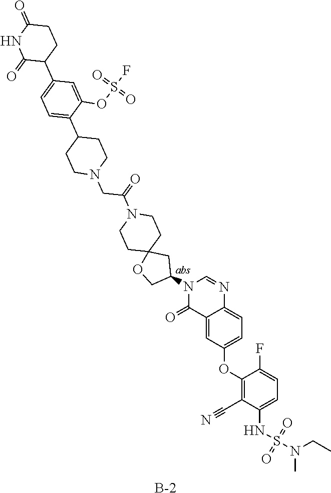 | *** | *** | *** |
| 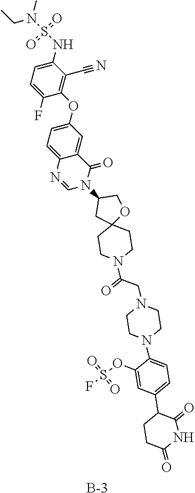 | ** | *** | *** |
| 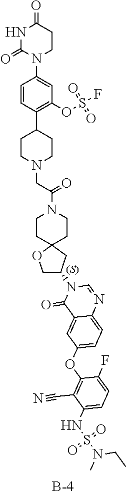 | * | * | * |
| 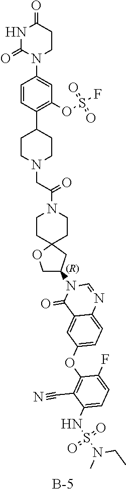 | * | * | * |
| 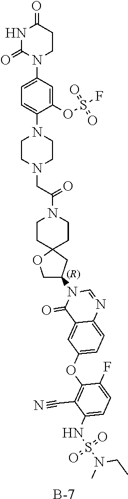 | * | * | * |
| 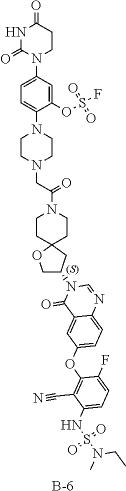 | * | ** | * |
| 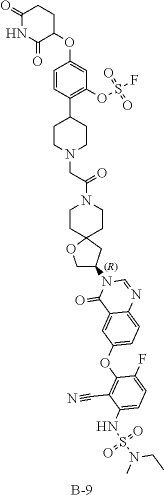 | ** | *** | *** |
| 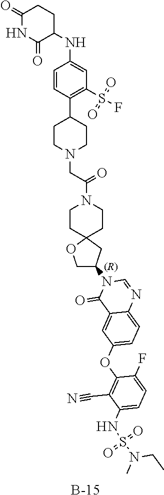 | * | ** | *** |
| 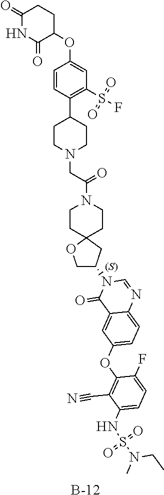 | ** | ** | *** |
| 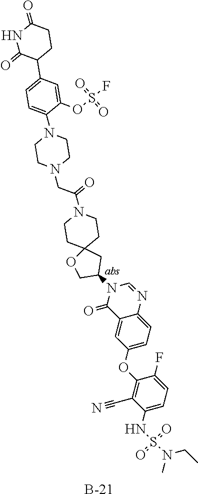 | * | *** | *** |
| 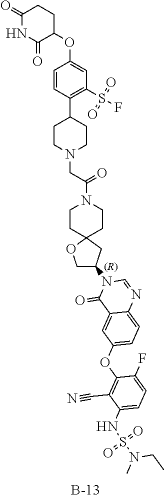 | ** | ** | *** |
| 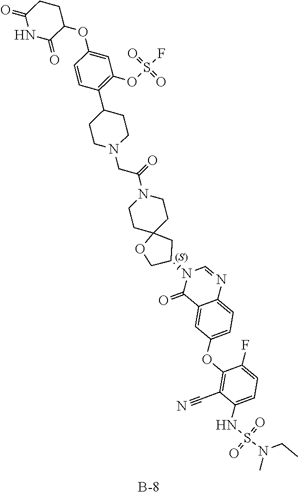 | ** | ** | *** |
| 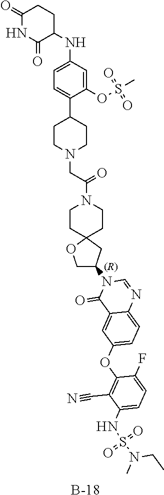 | ** | *** | *** |
| 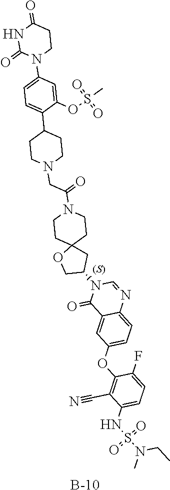 | * | *** | *** |
| 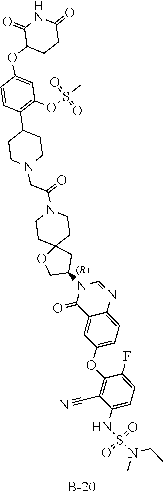 | ** | *** | *** |
| 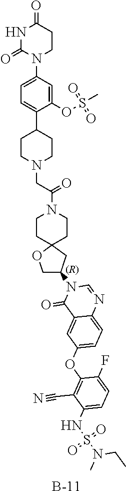 | ** | *** | ** |
| 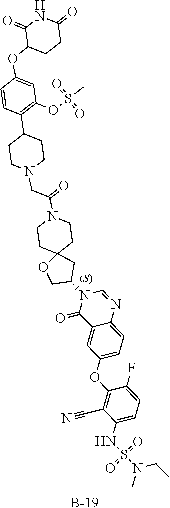 | * | ** | ** |
| 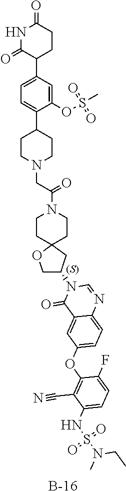 | * | *** | ** |
| 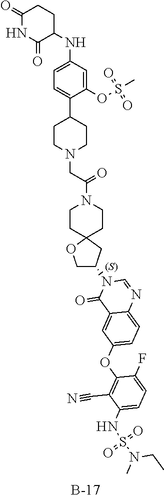 | * | ** | ** |
| 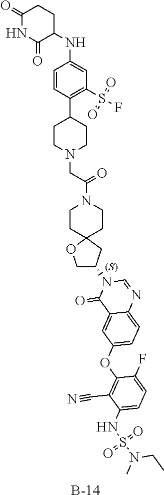 | * | ** | * |

In the table above for Emax >=95% is *; < 95% and >60% is **; <= 60% is
In the table above for DC₅₀ and IP >=10 μM is *; <10 μM and >1 μM is **; <= 1 μM is

TABLE 6
HiBiT-Degradation A375.10 BRAF 24 hours

| | HiBIT- | HiBIT- | HiBIT- |
|---|---|---|---|
| | Degradation | Degradation | Degradation |
| | A375.10 | A375.10 | A375.10 |
| | BRAF | BRAF | BRAF |
| | 24 hours | 24 hours | 24 hours |
| Compound | DC₅₀ nM | Emax | IP nM |
| 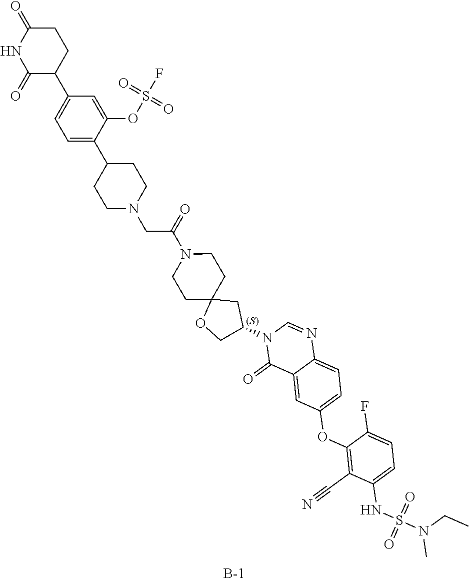 | *** | *** | *** |
| 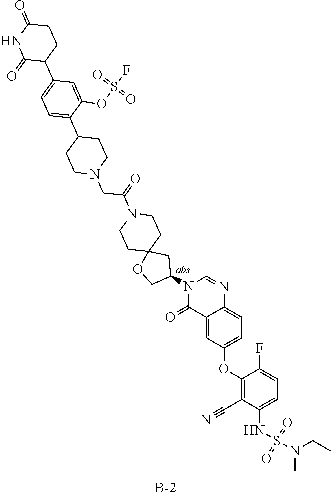 | *** | *** | *** |
| 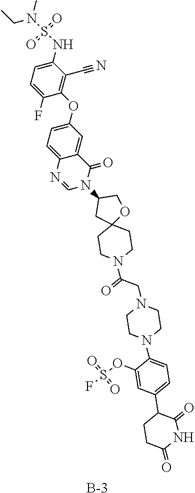 | *** | *** | *** |
| 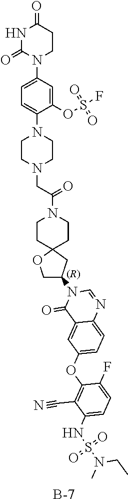 | ** | *** | *** |
| 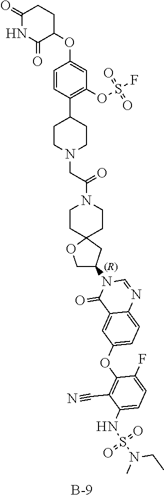 | *** | *** | *** |
| 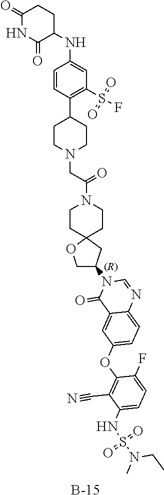 | *** | *** | *** |
| 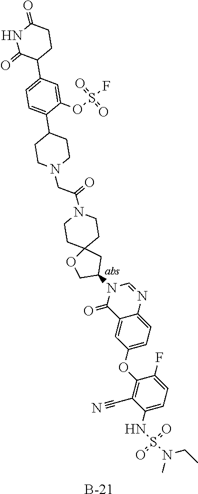 | *** | *** | *** |
| 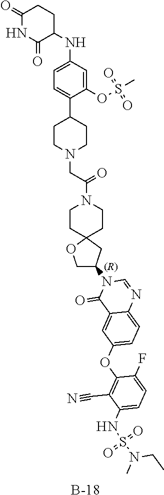 | *** | *** | *** |
| 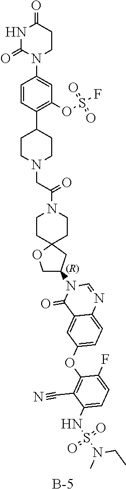 | * | ** | *** |
| 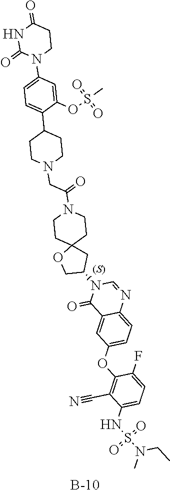 | *** | *** | *** |
| 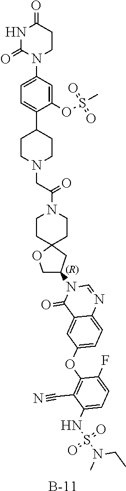 | *** | *** | *** |
| 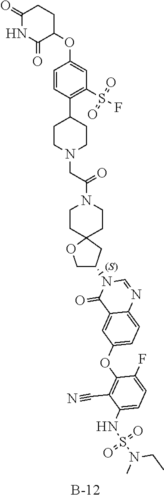 | *** | *** | *** |
| 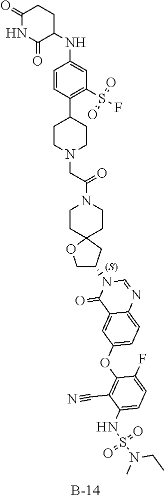 | ** | *** | *** |
| 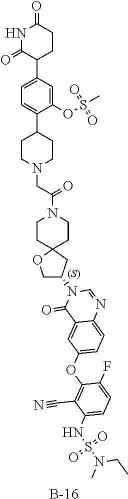 | *** | *** | *** |
| 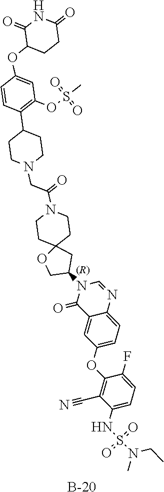 | ** | *** | *** |
| 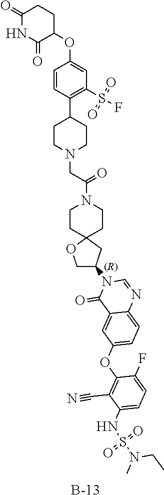 | * | ** | ** |
| 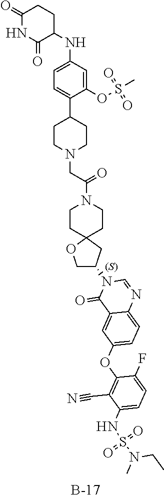 | ** | *** | ** |
| 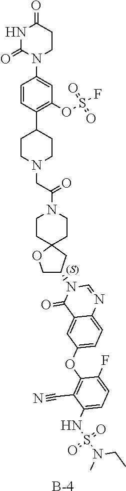 | ** | *** | ** |
| 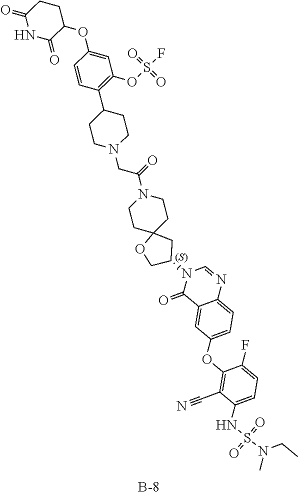 | ** | *** | ** |
| 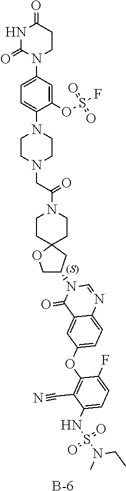 | ** | *** | ** |
| 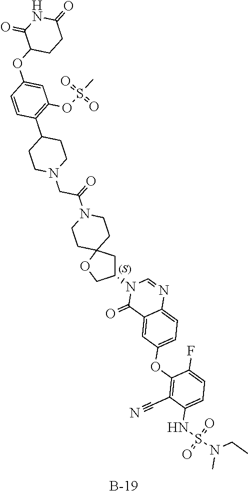 | * | *** | ** |

In the table above for Emax >=95% is *; < 95% and >60% is **; <=60% is ***
In the table above for DC50 and IP >=10 μM is *; <10 μM and >1 μM is **; <= 1 μM is ***

### EXAMPLE 7: GSPT1 HiBiT Degradation Assay

Selected compounds on the invention were tested in an assay evaluating the off-target degradation of the cereblon neosubstrate, Eukaryotic peptide chain release factor GTP-binding subunit ERF3A (GSPT1), as measured by luminescence using Nano-Glo HiBiT®Lytic Assay System (Catalog No. N3050, Promega, Madison, WI USA) in 293T cells CRISPR-edited to endogenously express GSPT1 with an N-terminal HiBiT fusion tag.

Wild-type 293T cells were obtained from ATCC (Catalog No.CRL-3216, Manassas, VA, USA), and the HiBiT-tagged cell line was generated by CRISPR knock-in into wild-type cells. The HiBiT polypeptide tag was introduced into the N-terminus of the endogenous GSPT 1 locus by CRISPR-Cas9 to produce a HiBiT-GSPT1 293T modified cell line (referred to here as 293T.114). Upon cell lysis and addition of the complementing LgBiT polypeptide and its substrate, the HiBiT polypeptide on GSPT1 allows reconstitution of an enzyme that activates its substrate to produce a luminescent signal that is directly proportional to the amount of HiBiT-tagged GSPT1. 293T.114 cells were maintained at 37° C. in an atmosphere of 5% CO2 in Dulbecco's modified Eagle Medium (DMEM) with high glucose (Catalog No. 11965092), supplemented with 10% fetal bovine serum (FBS; Catalog No. 10437036), and 10 mM HEPES (Catalog No. 15630130), all obtained from Thermofisher Scientific (Waltham, MA, USA). Cell culture flasks and 384-well microplates were acquired from VWR (Radnor, PA, USA).

Test compounds were prepared by dissolving neat compounds in DMSO (Catalog No.

D8418, Sigma-Aldrich, Inc., St. Louis, MO, USA) to generate 10 mM stock solutions, which were serially diluted (half log) in DMSO to generate an 11-point dose series (1000x final treatment concentration) in a 384-Well Low Dead Volume Microplate (Catalog No. LP-0200, Labcyte, San Jose, CA, USA). Using the Echo 550 Acoustic Liquid Handler (Labcyte), 30 nL of serially diluted compound solutions were dispensed in duplicate to 384-well TC-treated microplates (Catalog No. 3571, Coming, Glendale, CA, USA) and 30 nL of DMSO was dispensed to control wells. 293T.114 cells resuspended in growth media without phenol red were then added to 384-well plates containing the 11-point dose series of compounds in duplicate (maximum 1OuM concentration for final treatment) at a density of 6000 cells/well in 30u1. Plates were then spun briefly, and cells were incubated with compound at 37° C. for 24 hr.

Cellular GSPT1 protein level was determined based on quantification of luminescent signal using the Nano-Glo® HiBiT Lytic Assay kit (Promega). Following the 24 hr treatment time, Nano-Glo® HiBiT Lytic Assay reagents were added to the cells per the manufacturer's instructions by adding 30u1 of reagent per well, and the luminescence signal was acquired on the PHERAstar plate reader (BMG Labtech, Ortenberg, Germany). Cells treated with DMS0 only (no test compound treatment) were used as the negative control (N). Media only (no cells added) withNano-Glo reagent additionwasused as the background orpositive control (P). The percent response of compound treated samples were calculated by subtracting out the 5 background signal (P) and normalizing to the DMS0 control (N) as follows. 0% Response=100 x (Treated Signal - Average (P))/(Average (N) - Average (P)).

Data were analyzed with a 4-parameter logistic fit to generate sigmoidal dose-response curves. DC50 values were computed as the concentrations where the fitted curves cross the 5000 response level. The Emax, or maximum effect of each compound, represents the amount of 10 residual protein remaining in the cell following compound treatment. The IP, or inflection point of the dose-response curve, is the concentration of compound at which 50% o of the observed response is achieved.

TABLE 7
HiBiT-Degradation 293T.114 GSPT1 24 hours

| | HIBIT- | HIBIT- | HIBIT- |
|---|---|---|---|
| | Degradation | Degradation | Degradation |
| | 293T.114 | 293T.114 | 293T.114 |
| | GSPT1 | GSPT1 | GSPT1 |
| | 24 hours | 24 hours | 24 hours |
| Compound | DC₅₀ nM | Emax | IP nM |
| 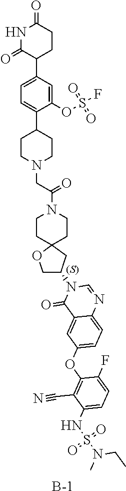 | * | *** | ** |
| 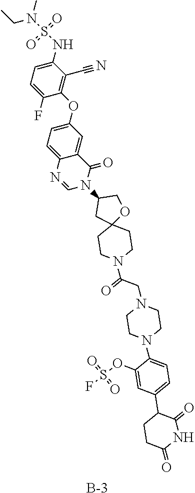 | * | ** | ** |

In the table above for Emax >=95% is *; <95% and >60% is **; <=60% is ***
In the table above for DC₅₀ and IP >=10 μM is *; <10 μM and >1 μM is **; <=1 μM is ***

### EXAMPLE 8: Covalent Ligand Bound to Cereblon - Crystal Structures

Co-crystallization trays of chimeric bacterial CRBN protein (11 mg/mL) were incubated with 2 mM of either A-1 or A-5 in 2% DMSO. The trays were chilled on ice for 1 hour before the ligands were dispensed. The ligands were dispensed with three ratios of protein: mother liquor at 1:.5, 1:1, 1:2 ratios in two different sparse matrix screens (PACT and PEGs-I). Crystal growth was monitored over the course of two weeks. Crystals of A-5 were harvested from a drop consisting of 0.2M diammonium tartrate and 20% PEG 3350 with 20% ethylene glycol being used as a cryoprotectant. Data was collected at the SOLEIL synchrotron on the Proxima-2a beamline yielding a 1.3A structure. Crystals of A-1 were harvested from a drop consisting of 0.14M dibasic potassium phosphate, 1.26M monobasic sodium phosphate monohydrate with a pH of 5.6 supplemented with 20% PEG400 as a cryoprotectant. Data for 15 A-1 was collected at the Diamond Light Source on the i04 beamline giving a 1.3A structure.

The crystallographic phases were generated using Phaser MR and structures were refined using iterative rounds of rebuilding in COOT and refinement in Refmac5. The crystal structures demonstrate that the two tested compounds covalently modify histidine 77 in bacterial cereblon which corresponds to histidine 353 in human cereblon.

The resulting crystal structures are provided in FIG. 81 and FIG. 82.

### Example 9: Covalent Labeling of CRBN and Analysis by Intact Protein Mass Spectrometry

Compound stability was assayed by LC-MS prior to analysis. New stocks were prepared if compound purity was <90% and solid stock was available; if no solid stock was available, compound stocks were used if purity was >80%.

Each compound was tested in duplicate at 15 μM and 150 μM. Compound A-5 was used as a control compound and was included in each sample set. To prepare samples, an Echo was used to dispense 150 nL of 1 mM or 10 mM compound stock in DMSO per well into a 384-well Protein LoBind plate (Eppendorf). Plates were stored at -20C until use, then were brought to room temperature prior to adding 10 μL of 15 μM protein stock (CRBN(delta 1-40)/DDB1(delta BPB)) to each well. The plate was sealed with adhesive foil and then was centrifuged to combine the protein and compound stock. Samples were incubated for 6 h at room temperature (70.5F) with shaking. To quench the reactions, 90 μL of 49% acetonitrile, 49% water, 2% formic acid, 10 mM DTT was added to each well. A 7K MWCO Zeba Spin Desalting plate (Thermo) was used to buffer exchange the samples into 49% acetonitrile, 49% water, 2% formic acid according to kit instructions.

Samples were analyzed using a Zipchip (908 Devices) attached to an Exploris 240 mass spectrometer (ThermoFisher Scientific). The Zipchip system was used with an HR chip and peptide BGE (both 908 Devices). The Zipchip method was 4 min long and used a 1.5 nL injection, 500 V/cm field strength, and a pressure-assist start time of 0.5 min. The Exploris method used an Orbitrap resolution of 60,000, a scan range of 500-3000 m/z, an RF Lens of 80%, a custom normalized AGC target of 100%, a custom maximum ion injection time of 20 ms, and 1 microscan. Data was collected in positive mode and profile format. No source fragmentation was performed.

Data was analyzed using the intact mass module in Byos (Protein Metrics). Peaks were detected with a smoothing width of 0.01, a minimum width of 0.05, and a flat baseline. Spectra were deconvoluted over a mass range of 40,000 to 100,000 Da and a m/z range of 500 to 4000.

For automass peak picking, the minimum difference between mass peaks was 15 Da and the maximum number of mass peaks was 10. Peaks were assigned to CRBN with 0 disulfides

TABLE 8
Covalent Labeling of CRBN

| Compound # | % modified (150 μM) | % modified (15 μM) |
|---|---|---|
| A-1 | ++++ | ++++ |
| A-2 | ++++++ | ++++ |
| A-3 | + | + |
| A-4 | ++++++ | +++++ |
| A-5 | +++++ | ++++ |
| A-6 | ++++++ | ++++++ |
| A-7 | ++++++ | +++++ |
| A-8 | +++++++ | ++++ |
| A-10 | +++++ | +++++ |
| A-11 | ++++++ | +++++ |
| A-12 | ++++++ | +++++ |
| A-13 | +++++ | ++++ |
| A-14 | +++++++ | +++++ |
| A-15 | + | + |
| A-16 | +++++++ | +++++++ |
| A-17 | +++++ | ++++ |
| A-18 | + | + |
| A-19 | +++++++ | ++++ |
| A-20 | ++++ | +++ |
| A-21 | +++++ | +++ |
| A-22 | +++ | ++ |
| A-23 | + | + |
| A-24 | ++++++ | +++++ |
| A-25 | ++ | + |
| A-26 | + | + |
| A-27 | ++++++ | + |
| A-28 | + | + |
| A-29 | + | + |
| A-30 | ++++ | + |
| A-33 | + | + |
| A-34 | ++ | + |
| A-35 | + | + |
| A-36 | + | + |
| A-37 | + | + |
| A-38 | + | + |
| A-40 | + | + |
| A-42 | ++++++ | ++++ |
| A-45 | +++++++ | ++++++ |
| A-46 | + | + |
| A-47 | + | + |
| A-49 | ++++ | + |
| A-53 | + | + |
| A-55 | + | + |
| A-58 | +++++++ | +++++ |
| A-59 | + | + |
| A-60 | +++++++ | ++++ |
| A-61 | ++ | + |
| A-62 | +++++++ | +++++++ |
| A-63 | + | + |
| A-64 | + | + |
| A-65 | + | + |
| A-66 | ++ | + |

As used in the table above for % modified: <5% = +, ≥5% and <10% = ++, ≥10% and <20% = +++, ≥20% and <40%= ++++, ≥40% and <60% = +++++, ≥60% and <80% = ++++++, ≥80% = +++++++.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teaching of this invention that certain changes and modifications may be made thereto without departing from the spirit or scope of the invention as defined in the appended claims. Additionally, those skilled in the art will recognize, or be able to ascertain using no more than routine experimentation, many equivalents to the specific embodiments and methods described herein. Such equivalents are intended to be encompassed by the scope of the present application.

We claim:

1. A compound of Formula: 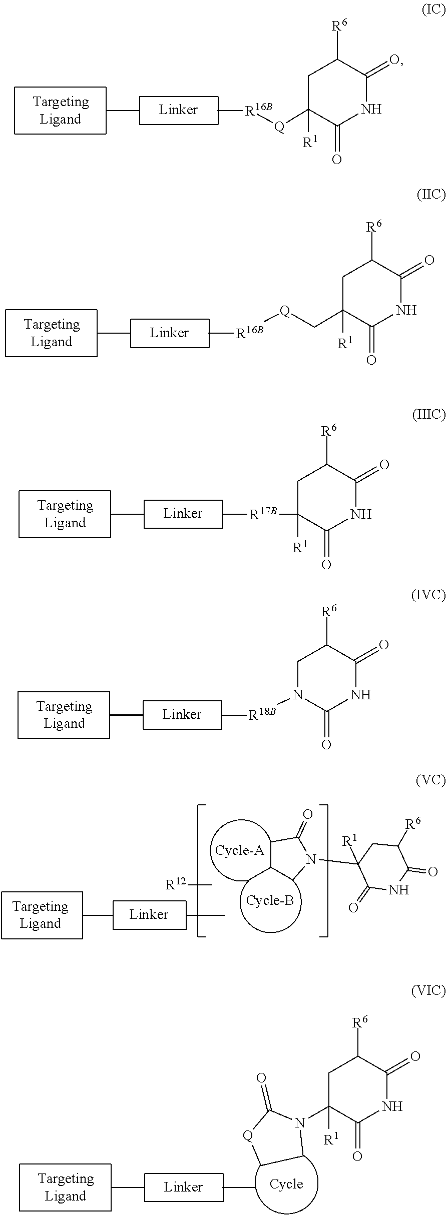 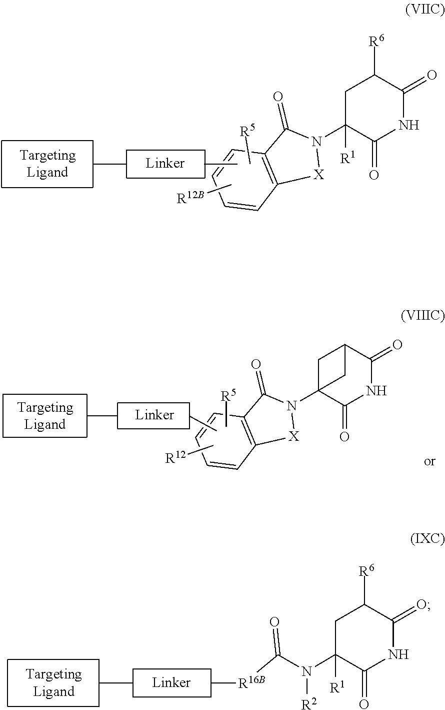
or a pharmaceutically acceptable salt thereof,
wherein:
Q is CH₂, NR², O, or S;
X is CH₂ or C(O);
R¹ and R⁶ are independently selected from hydrogen, alkyl, alkenyl, alkynyl, and halogen;
or R¹ and R⁶ are combined to form a one or two carbon bridge to form a fused cycle;
each R² is selected from hydrogen, alkyl, haloalkyl, alkenyl, alkynyl, aryl, heteroaryl, heterocycle, and —C(O)R⁹, each of which is optionally substituted with 1 or 2 substituents independently selected from R¹⁰;
each R⁵, R^{5a}, and R^{5b} is independently selected from hydrogen, alkyl, haloalkyl, alkenyl, alkynyl, halogen, aryl, heteroaryl, heterocycle, cyano, nitro, —NR⁷R⁸, —OR⁷, —SR⁷, —C(O)R⁹, —C(S)R⁹, —S(O)R⁹, —S(O)₂R⁹, and —P(O)(R⁹)₂; each of which except hydrogen, halogen, cyano, and nitro is optionally substituted with 1 or 2 substituents independently selected from R^{10a};
Linker is of Formula: 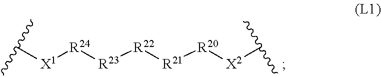
X¹ and X² are independently at each occurrence selected from bond, carbamate, heterocycle, NR⁴², C(R⁴²)₂, O, C(O), and S;
R⁴² is independently at each occurrence selected from the group consisting of hydrogen, alkyl, heteroalkyl, heterocycle, aryl, heteroaryl, —C(O)H, —C(O)OH, —C(O)alkyl, —C(O)Oalkyl, —C(O)(aryl, heteroalkyl or heteroaryl), —C(O)O(aryl, heteroalkyl, or heteroaryl), alkenyl, and alkynyl;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkenyl, alkynyl, haloalkyl, alkoxy, aryl, heterocycle, heteroaryl, and carbocycle; each of which is optionally substituted with 1, 2, or 3 substituents independently selected from R⁴⁰;
R²⁶ is independently at each occurrence selected from the group consisting of hydrogen, alkyl, arylalkyl, heteroarylalkyl, alkenyl, alkynyl, aryl, heteroaryl, and heterocycle;
R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkenyl, and alkynyl;
R⁴⁰ is independently at each occurrence selected from the group consisting of hydrogen, alkyl, alkenyl, alkynyl, fluoro, bromo, chloro, azide, OR⁷, NR⁷R⁸, S(O)NR⁷R⁸, S(O)₂NR⁷R⁸, S(O)OR⁷, S(O)2OR⁷, haloalkyl, aryl, heteroaryl, heterocycle, and cycloalkyl;
R⁴¹ is independently at each occurrence selected from the group consisting of hydrogen, alkyl, alkenyl, alkynyl, haloalkyl, aryl, heteroaryl, heterocycle, and cycloalkyl;
R^{16B} is selected from: 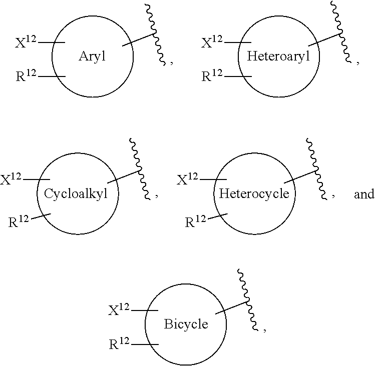
each of which is optionally substituted with 1 or 2 substituents independently selected from R⁵;
R^{17B} is selected from: 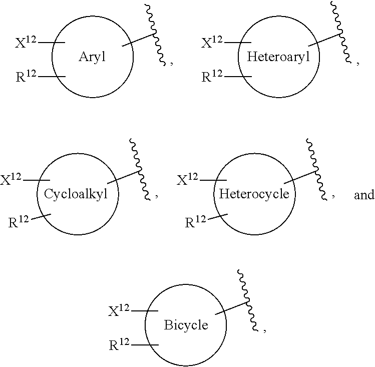
each of which is optionally substituted with 1 or 2 substituents independently selected from R⁵;
R^{18B} is selected from: 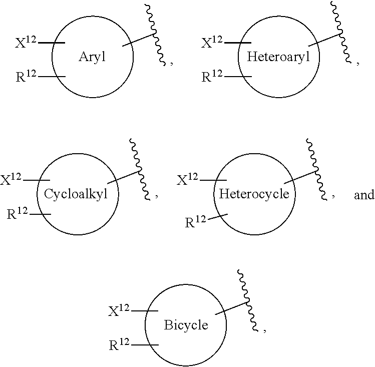
each of which is attached to the azaglutarimide moiety through a C—N bond and each of which R^{18B} is optionally substituted with 1 or 2 substituents independently selected from R⁵;
X¹² is the attachment point to Linker;
Cycle is a fused aryl or heteroaryl group optionally substituted with 1 or 2 substituents independently selected from R⁵ and substituted with one R¹² substituent;
Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1 or 2 substituents independently selected from R^{5a}.
Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1 or 2 substituents independently selected from R^{5b};
R¹² is selected from 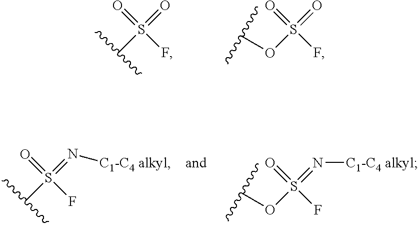
R¹²B is selected from 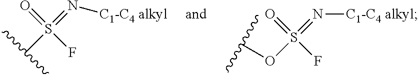
R⁷ and R⁸ at each instance are independently selected from hydrogen alkyl, haloalkyl, alkenyl, alkynyl, aryl, heteroaryl, heterocycle, and C(O)R¹⁴ each of which except hydrogen is optionally substituted with 1 or 2 substituents independently selected from R^{10b};
each R⁹ is independently selected from hydrogen, alkyl, haloalkyl, alkenyl, alkynyl, aryl, heteroaryl, heterocycle, —NR⁷R⁸, —OR⁷, and —SR⁷;
each R¹⁰, R^{10a}, and R^{10b} is independently selected from hydrogen, alkyl, haloalkyl, alkenyl, alkynyl, halogen, aryl, heteroaryl, heterocycle, cyano, nitro, —NR¹¹R¹³, —OR¹¹, —SR¹¹, —C(O)R¹⁴, —C(S)R¹⁴, —S(O)R¹⁴, —S(O)₂R¹⁴, and —P(O)(R¹⁴)₂;
R¹¹ and R¹³ at each instance are independently selected from hydrogen, alkyl, haloalkyl, alkenyl, alkynyl, aryl, heteroaryl, heterocycle, —C(O)R¹⁴, —C(S)R¹⁴, —S(O)R¹⁴, —S(O)₂R¹⁴, and —P(O)(R¹⁴)₂; each of which except hydrogen is optionally substituted with 1 or 2 substituents independently selected from R¹⁵;
each R¹⁴ is independently selected from hydrogen, alkyl, haloalkyl, alkenyl, alkynyl, aryl, heteroaryl, heterocycle, amino, hydroxyl, alkoxy, —N(H)(alkyl), and —N(alkyl)_{2;}
each R¹⁵ is independently selected from hydrogen, alkyl, haloalkyl, alkenyl, alkynyl, halogen, aryl, heteroaryl, heterocycle, cyano, nitro, amino, hydroxyl, alkoxy, —N(H)(alkyl), and —N(alkyl)₂; and
Targeting Ligand is a ligand that binds to a Target Protein.

2. The compound of claim 1, wherein the compound is of Formula: 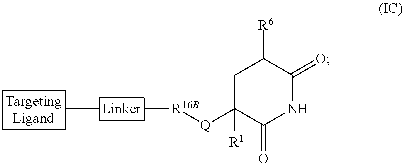
or a pharmaceutically acceptable salt thereof.

3. The compound of claim 1, wherein the compound is of Formula: 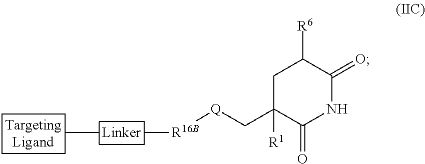
or a pharmaceutically acceptable salt thereof.

4. The compound of claim 2, wherein Q is NH.

5. The compound of claim 2, wherein Q is O.

6. The compound of claim 2, wherein Q is S.

7. The compound of claim 1, wherein the compound is of Formula: 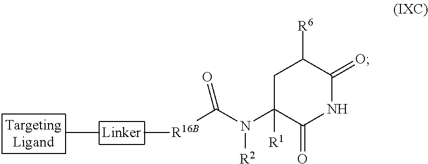
or a pharmaceutically acceptable salt thereof.

8. The compound of claim 7, wherein R² is hydrogen.

9. The compound of claim 1, wherein the compound is of Formula: 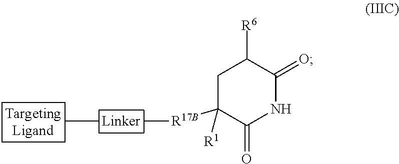
or a pharmaceutically acceptable salt thereof.

10. The compound of claim 1, wherein the compound is of Formula: 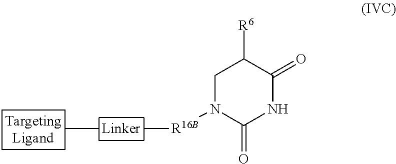
or a pharmaceutically acceptable salt thereof.

11. The compound of claim 1, wherein R^{16B}, R^{17B} and R^{18B} are 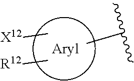
optionally substituted with 1 or 2 substituents independently selected from R⁵.

12. The compound of claim 1, wherein R^{16B}, R^{17B} and R^{18B} are 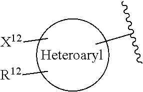
optionally substituted with 1 or 2 substituents independently selected from R⁵.

13. The compound of claim 1, wherein the compound is of Formula: 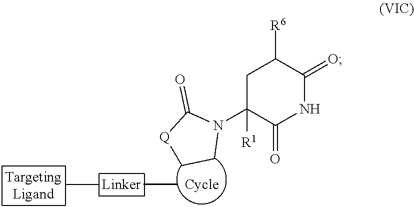
or a pharmaceutically acceptable salt thereof.

14. The compound of claim 13, wherein Q is NH.

15. The compound of claim 13, wherein Q is NCH₃.

16. The compound of claim 13, wherein Q is CH₂.

17. The compound of claim 13, wherein Q is O.

18. The compound of claim 13, wherein Cycle is phenyl optionally substituted with 1 or 2 substituents independently selected from R⁵ and substituted with one R¹² substituent.

19. The compound of claim 1, wherein the compound is of Formula: 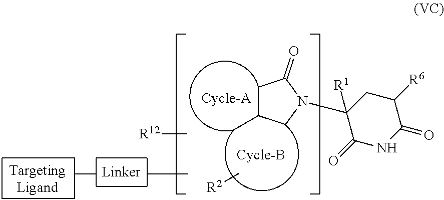
or a pharmaceutically acceptable salt thereof.

20. The compound of claim 19, wherein Cycle-A is phenyl optionally substituted with 1 or 2 substituents independently selected from R^{5a}.

21. The compound of claim 20, wherein Cycle-B is phenyl optionally substituted with 1 or 2 substituents independently selected from R^{5b}.

22. The compound of claim 1, wherein R¹² is

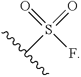 [SMILES: *S(=O)(=O)F]

23. The compound of claim 1, wherein R¹² is

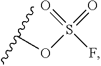 [SMILES: *OS(=O)(=O)F]

24. The compound of claim 1, wherein R¹² is

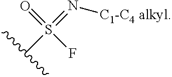

25. The compound of claim 1, wherein R¹² is

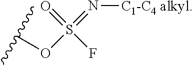

26. The compound of claim 1, wherein R⁶ is hydrogen.

27. The compound of claim 1, wherein R¹ is hydrogen.

28. The compound of claim 1, wherein the compound is selected from the group consisting of: 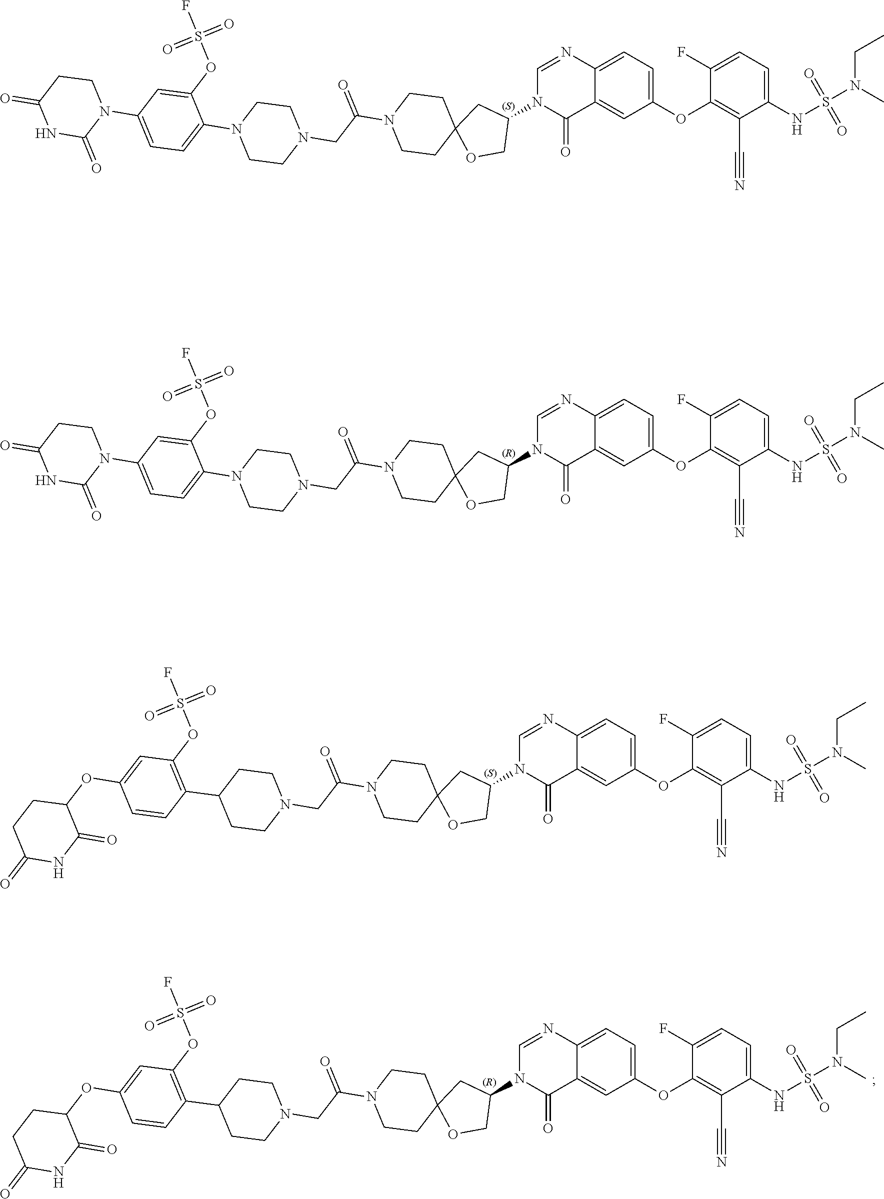
or a pharmaceutically acceptable salt thereof.

29. The compound of claim 1, wherein the compound is selected from the group consisting of: 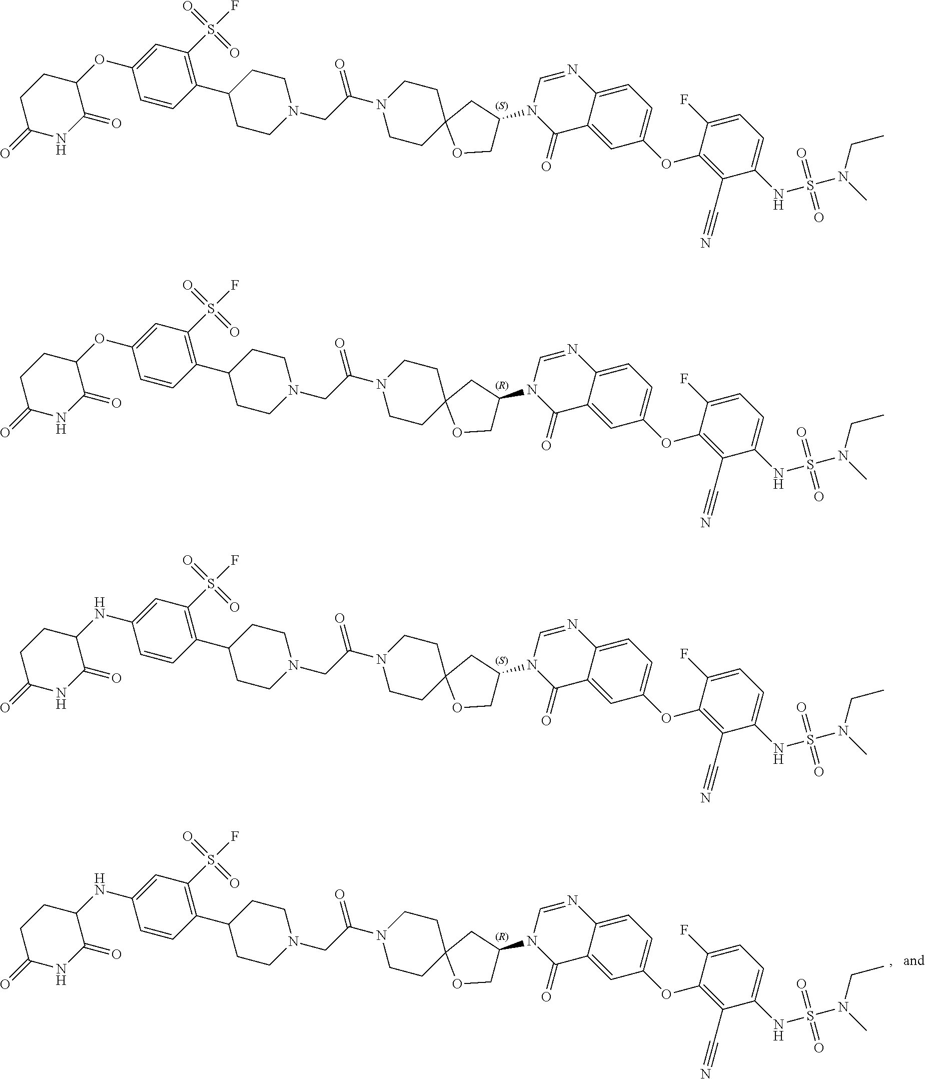 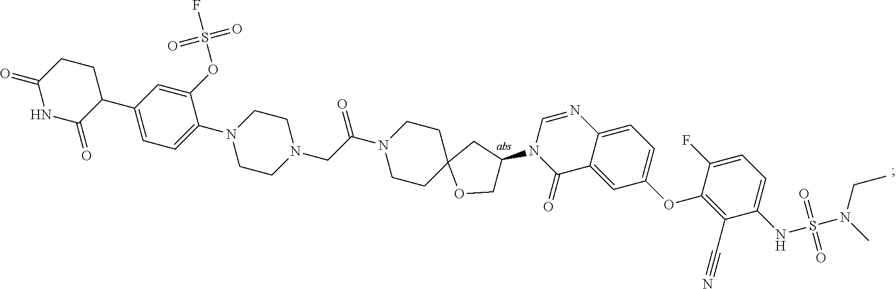
or a pharmaceutically acceptable salt thereof.

30. The compound of claim 1, wherein the compound is selected from the group consisting of: 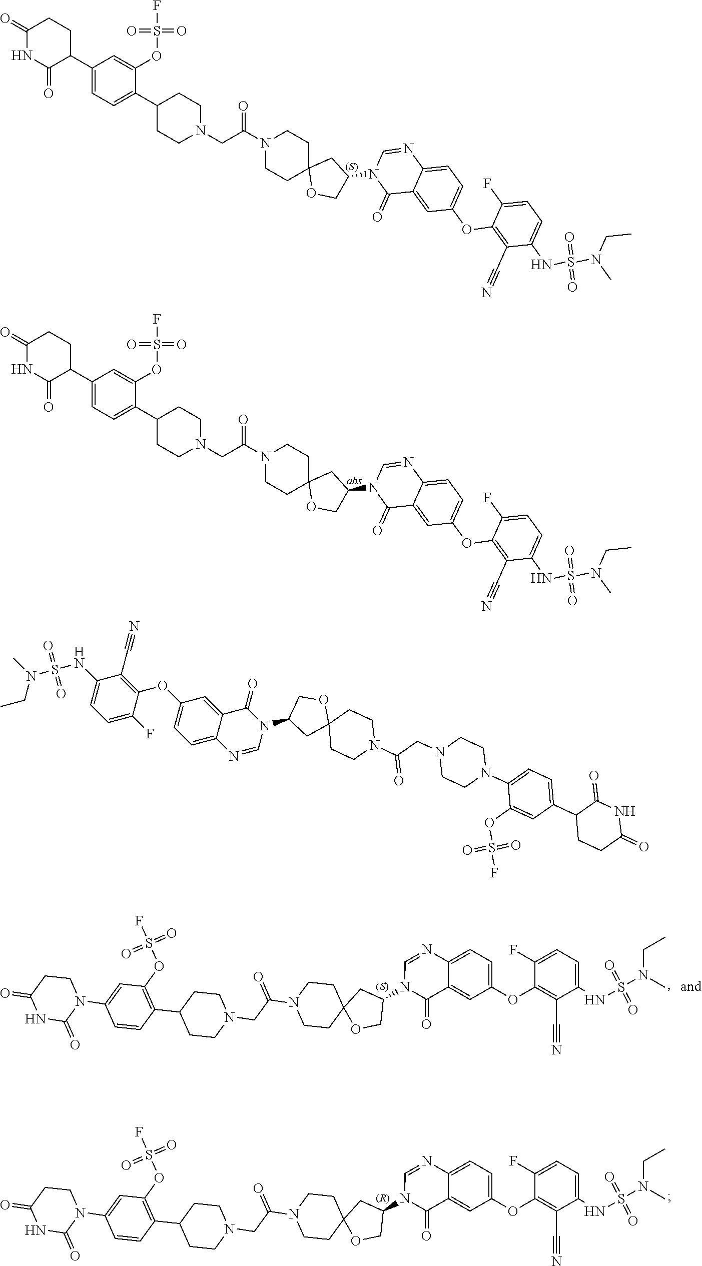
or a pharmaceutically acceptable salt thereof.
